(19) United States
(12) Patent Application Publication
(10) Pub. No.: US 2024/0415013 A1
(43) Pub. Date: Dec. 12, 2024

(54) ORGANIC ELECTROLUMINESCENT ELEMENT AND ELECTRONIC DEVICE

(71) Applicant: IDEMITSU KOSAN CO.,LTD., Tokyo (JP)

(72) Inventors: Masato NAKAMURA, Tokyo (JP); Satomi TASAKI, Tokyo (JP); Tasuku HAKETA, Tokyo (JP); Emiko KAMBE, Tokyo (JP); Keitaro YAMADA, Tokyo (JP); Tetsuya MASUDA, Tokyo (JP); Yusuke TAKAHASHI, Tokyo (JP); Hiroaki TOYOSHIMA, Tokyo (JP); Takeshi IKEDA, Tokyo (JP); Kazuki NISHIMURA, Tokyo (JP)

(73) Assignee: IDEMITSU KOSAN CO.,LTD., Tokyo (JP)

(21) Appl. No.: 18/697,130

(22) PCT Filed: Sep. 30, 2022
(86) PCT No.: PCT/JP2022/036718

§ 371 Date: Mar. 29, 2024

(30) Foreign Application Priority Data
Oct. 1, 2021 (JP) 2021-163148

(51) Int. Cl. H10K 85/60; C09K 11/02; C09K 11/06; H10K 50/12; H10K 50/15; H10K 101/00

(52) U.S. Cl. CPC H10K 85/633; C09K 11/02; C09K 11/06; H10K 85/631; H10K 85/636; H10K 85/658; C09K 2211/1007; C09K 2211/1011; C09K 2211/1014; C09K 2211/1022; H10K 50/12; H10K 50/156; H10K 85/622; H10K 85/626; H10K 85/6572; H10K 85/6574; H10K 2101/90

(57) ABSTRACT

An organic EL device includes an emitting region between a cathode and an anode, a first anode side organic layer, and a second anode side organic layer, in which the emitting region includes at least a first emitting layer, the first emitting layer contains a first host material, a first additional host material, and a first luminescent compound, the first anode side organic layer contains first and second organic materials, a content of the first organic material is less than 50 mass %, the second anode side organic layer contains a second hole transporting zone material, the first emitting layer is an emitting layer that emits fluorescence, and a refractive index NM₁ of constituent materials in the first anode side organic layer and a refractive index NM₂ of a constituent material in the second anode side organic layer satisfy a relationship of Numerical Formula NM,

$${NM}_{1} > {NM}_{2} . \qquad ( Numerical Formula NM )$$

### TECHNICAL FIELD

The present invention relates to an organic electroluminescence device and an electronic device.

### BACKGROUND ART

An organic electroluminescence device (hereinafter, occasionally referred to as “organic EL device”.) has found its application in a full-color display for mobile phones, televisions, and the like. When voltage is applied to an organic EL device, holes are injected from an anode and electrons are injected from a cathode into an emitting layer. The injected holes and electrons are recombined in the emitting layer to form excitons. Specifically, according to the electron spin statistics theory, singlet excitons and triplet excitons are generated at a ratio of 25%:75%.

In Patent Literatures 1 to 5, for instance, studies have been made to improve performance of an organic EL device. The performance of the organic EL device is evaluable in terms of, for instance, luminance, emission wavelength, chromaticity, luminous efficiency, drive voltage, and lifetime.

Patent Literatures 1 to 5 disclose an organic EL device in which a hole transporting zone includes a plurality of layers.

### CITATION LIST

### Patent Literature(s)

Patent Literature 1 International Publication No. WO 2020/189316
Patent Literature 2 JP 2019-161218 A
Patent Literature 3 International Publication No. WO 2011/093056
Patent Literature 4 US Patent Application Publication No. 2021/0159418
Patent Literature 5 US Patent Application Publication No. 2021/234098

### SUMMARY OF THE INVENTION

### Problem(s) to be Solved by the Invention

An object of the invention is to provide an organic electroluminescence device excellent in luminous efficiency and an electronic device including the organic electroluminescence device.

### Means for Solving the Problem(s)

According to an aspect of the invention, there is provided an organic electroluminescence device, including a cathode; an anode; an emitting region disposed between the cathode and the anode; and a hole transporting zone disposed between the anode and the emitting region, in which the emitting region includes one or more emitting layers, the one or more emitting layers include a first emitting layer, the first emitting layer contains a first host material, a first additional host material different from the first host material, and a first luminescent compound, the hole transporting zone includes a first anode side organic layer and a second anode side organic layer, the first anode side organic layer is in direct contact with the second anode side organic layer, the first anode side organic layer and the second anode side organic layer are disposed between the anode and the emitting region in this order from a side close to the anode, the first anode side organic layer contains a first organic material and a second organic material, the first organic material is different from the second organic material, a content of the first organic material in the first anode side organic layer is less than 50 mass %, the second organic material is a monoamine compound having one substituted or unsubstituted amino group in a molecule, or a diamine compound having two substituted or unsubstituted amino groups in a molecule, the second anode side organic layer contains a second hole transporting zone material, the second hole transporting zone material is a monoamine compound having one substituted or unsubstituted amino group in a molecule, or a diamine compound having two substituted or unsubstituted amino groups in a molecule, the second hole transporting zone material is different from the first organic material, the second hole transporting zone material and the second organic material are mutually the same or different, the first emitting layer is an emitting layer that emits fluorescence, and a refractive index NM₁ of constituent materials contained in the first anode side organic layer and a refractive index NM₂ of a constituent material contained in the second anode side organic layer satisfy a relationship of a numerical formula below (Numerical Formula NM).

$${NM}_{1} > {NM}_{2} \qquad ( Numerical Formula NM )$$

According to another aspect of the invention, there is provided an organic electroluminescence device, including a cathode; an anode; an emitting region disposed between the cathode and the anode; a hole transporting zone disposed between the anode and the emitting region, and an electron transporting zone disposed between the cathode and the emitting region, in which the emitting region includes one or more emitting layers, the one or more emitting layers include a first emitting layer, the first emitting layer contains a first host material, a first additional host material different from the first host material, and a first luminescent compound, the electron transporting zone includes at least one electron transporting layer, the hole transporting zone includes a first anode side organic layer and a second anode side organic layer, the first anode side organic layer is in direct contact with the second anode side organic layer, the first anode side organic layer and the second anode side organic layer are disposed between the anode and the emitting region in this order from a side close to the anode, the first anode side organic layer contains a first organic material and a second organic material, the first organic material is different from the second organic material, a content of the first organic material in the first anode side organic layer is less than 50 mass %, the second organic material is a monoamine compound having one substituted or unsubstituted amino group in a molecule, or a diamine compound having two substituted or unsubstituted amino groups in a molecule, the second anode side organic layer contains a second hole transporting zone material, the second hole transporting zone material is a monoamine compound having one substituted or unsubstituted amino group in a molecule, or a diamine compound having two substituted or unsubstituted amino groups in a molecule, the second hole transporting zone material is different from the first organic material, the second hole transporting zone material and the second organic material are mutually the same or different, the at least one electron transporting layer in the electron transporting zone contains a phenanthroline compound having a phenanthroline skeleton, the phenanthroline compound is a compound that has at least one group represented by a formula (21) below and is represented by a formula (20) below, the first emitting layer is an emitting layer that emits fluorescence, and a refractive index NM₁ of constituent materials contained in the first anode side organic layer and a refractive index NM₂ of a constituent material contained in the second anode side organic layer satisfy a relationship of a numerical formula below (Numerical Formula NM).

$${NM}_{1} > {NM}_{2} \qquad ( Numerical Formula NM )$$

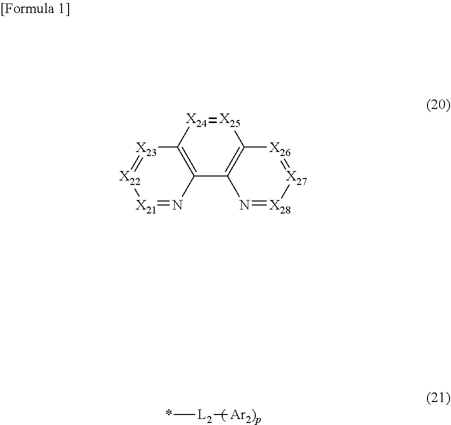

In the formula (20):
X₂₁ to X₂₈ are each independently a nitrogen atom, CR₂₁, or a carbon atom bonded to a group represented by the formula (21);
at least one of X₂₁ to X₂₈ is a carbon atom bonded to a group represented by the formula (21);
when a plurality of groups represented by the formula (21) are present, the plurality of groups represented by the formula (21) are mutually the same or different;
at least one combination of adjacent two or more of a plurality of R₂₁ are mutually bonded to form a substituted or unsubstituted monocyclic ring, mutually bonded to form a substituted or unsubstituted fused ring, or not mutually bonded; and
each R₂₁ forming neither the substituted or unsubstituted monocyclic ring nor the substituted or unsubstituted fused ring is independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₉₃₁, a group represented by —COOR₉₃₂, a group represented by —S(═O)₂R₉₃₃, a group represented by —B(R₉₃₄)(R₉₃₅), a group represented by —P(═O)(R₉₃₆)(R₉₃₇), a halogen atom, a cyano group, a nitro group, a substituted or unsubstituted aryl group having 6 to 50 ring carbon atoms, or a substituted or unsubstituted heterocyclic group having 5 to 50 ring atoms.

In the formula (21):
Ar₂ is a substituted or unsubstituted aryl group having 6 to 50 ring carbon atoms, or a substituted or unsubstituted heterocyclic group having 5 to 50 ring atoms;
p is 1, 2, 3, 4, or 5;
when two or more Ar₂ are present, the two or more Ar₂ are mutually the same or different;
L₂ is a single bond or a linking group;
L₂ as the linking group is a substituted or unsubstituted polyvalent linear, branched or cyclic aliphatic hydrocarbon group having 1 to 50 carbon atoms, a substituted or unsubstituted polyvalent aromatic hydrocarbon group having 6 to 50 ring carbon atoms, a substituted or unsubstituted polyvalent heterocyclic group having 5 to 50 ring atoms, or a polyvalent multiple linking group provided by bonding two or three groups selected from the polyvalent aromatic hydrocarbon ring group and the polyvalent heterocyclic group;
the aromatic hydrocarbon ring group and the heterocyclic group forming L₂ as the multiple linking group are mutually the same or different, and adjacent ones thereof are mutually bonded to form a ring, or not mutually bonded;
Ar₂ and L₂ as the linking group are mutually bonded to form a ring, or not mutually bonded;
L₂ as the linking group, and a carbon atom in one of X₂₁ to X₂₈ adjacent to a carbon atom bonded to L₂, or R₂₁ in CR₂₁ are mutually bonded to form a ring, or not mutually bonded; and
* in the formula (21) represents a bonding position to a ring represented by the formula (20);
in the phenanthroline compound, R₉₀₁, R₉₀₂, R₉₀₃, R₉₀₄, R₉₀₅, R₉₀₆, R₉₀₇, R₉₃₁, R₉₃₂, R₉₃₃, R₉₃₄, R₉₃₅, R₉₃₆ and R₉₃₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different;
when a plurality of R₉₀₇ are present, the plurality of R₉₀₇ are mutually the same or different;
when a plurality of R₉₃₁ are present, the plurality of R₉₃₁ are mutually the same or different;
when a plurality of R₉₃₂ are present, the plurality of R₉₃₂ are mutually the same or different;
when a plurality of R₉₃₃ are present, the plurality of R₉₃₃ are mutually the same or different;
when a plurality of R₉₃₄ are present, the plurality of R₉₃₄ are mutually the same or different;
when a plurality of R₉₃₅ are present, the plurality of R₉₃₅ are mutually the same or different;
when a plurality of R₉₃₆ are present, the plurality of R₉₃₆ are mutually the same or different; and
when a plurality of R₉₃₇ are present, the plurality of R₉₃₇ are mutually the same or different.

According to still another aspect of the invention, there is provided an organic electroluminescence device, including a cathode; an anode; an emitting region disposed between the cathode and the anode; and a hole transporting zone disposed between the anode and the emitting region, in which the emitting region includes one or more emitting layers, the one or more emitting layers include a first emitting layer, the first emitting layer contains a first host material, a first additional host material different from the first host material, and a first luminescent compound, the first host material is a compound represented by a formula (H10) below, the first additional host material is a compound represented by a formula (H20) below, the compound represented by the formula (H20) has at least one deuterium atom, the hole transporting zone includes a first anode side organic layer and a second anode side organic layer, the first anode side organic layer is in direct contact with the second anode side organic layer, the first anode side organic layer and the second anode side organic layer are disposed between the anode and the emitting region in this order from a side close to the anode, the first anode side organic layer contains a first organic material and a second organic material, the first organic material is different from the second organic material, a content of the first organic material in the first anode side organic layer is less than 50 mass %, the second organic material is a monoamine compound having one substituted or unsubstituted amino group in a molecule, or a diamine compound having two substituted or unsubstituted amino groups in a molecule, the second anode side organic layer contains a second hole transporting zone material, the second hole transporting zone material is a monoamine compound having one substituted or unsubstituted amino group in a molecule, or a diamine compound having two substituted or unsubstituted amino groups in a molecule, the second hole transporting zone material is different from the first organic material, the second hole transporting zone material and the second organic material are mutually the same or different, the first emitting layer is an emitting layer that emits fluorescence, and a refractive index NM₁ of constituent materials contained in the first anode side organic layer and a refractive index NM₂ of a constituent material contained in the second anode side organic layer satisfy a relationship of a numerical formula below (Numerical Formula NM).

$${NM}_{1} > {NM}_{2} \qquad ( Numerical Formula NM )$$

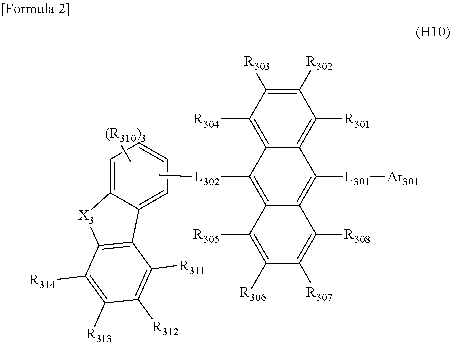

In the formula (H10):
X₃ is an oxygen atom or a sulfur atom;
at least one combination of adjacent two or more of three R₃₁₀ are mutually bonded to form a substituted or unsubstituted monocyclic ring, mutually bonded to form a substituted or unsubstituted fused ring, or not mutually bonded;
at least one combination of adjacent two or more of R₃₁₁ to R₃₁₄ are mutually bonded to form a substituted or unsubstituted monocyclic ring, or mutually bonded to form a substituted or unsubstituted fused ring;
R₃₀₁ to R₃₀₈, and R₃₁₀ to R₃₁₄ forming neither the substituted or unsubstituted monocyclic ring nor the substituted or unsubstituted fused ring are each a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms;
L₃₀₁ and L₃₀₂ are each independently a single bond, a substituted or unsubstituted arylene group having 6 to 50 ring carbon atoms, or a substituted or unsubstituted divalent heterocyclic group having 5 to 50 ring atoms; and
Ar₃₀₁ is a substituted or unsubstituted aryl group having 6 to 50 ring carbon atoms, or a substituted or unsubstituted heterocyclic group having 5 to 50 ring atoms.

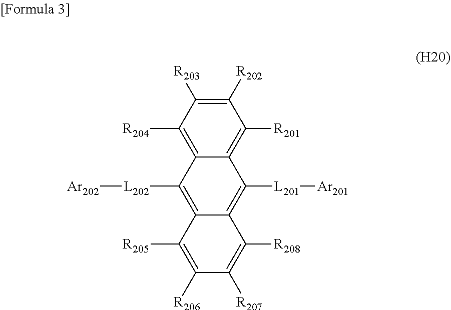

In the formula (H20):
R₂₀₁ to R₂₀₈ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a group represented by a formula (H21) below.

[Formula 4]

-L₂₀₃-Ar₂₀₃ (H21)

In the formulae (H20) and (H21):
L₂₀₁, L₂₀₂, and L₂₀₃ are each independently a single bond, or a substituted or unsubstituted arylene group having 6 to 50 ring carbon atoms; and
Ar₂₀₁, Ar₂₀₂, and Ar₂₀₃ are each independently a substituted or unsubstituted aryl group having 6 to 50 ring carbon atoms, or a substituted or unsubstituted heterocyclic group having 5 to 50 ring atoms.

In the first host material and the first additional host material, R₉₀₁, R₉₀₂, R₉₀₃, R₉₀₄, R₉₀₅, R₉₀₆ and R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different; and
when a plurality of R₉₀₇ are present, the plurality of R₉₀₇ are mutually the same or different.

According to a further aspect of the invention, there is provided an electronic device including the organic electroluminescence device according to the above aspect of the invention.

According to the aspects of the invention, there can be provided an organic electroluminescence device excellent in luminous efficiency and an electronic device including the organic electroluminescence device.

### BRIEF EXPLANATION OF DRAWINGS

FIG. 1 schematically depicts an exemplary arrangement of an organic electroluminescence device according to a first exemplary embodiment.

FIG. 2 schematically depicts another exemplary arrangement of the organic electroluminescence device according to the first exemplary embodiment.

FIG. 3 schematically depicts still another exemplary arrangement of the organic electroluminescence device according to the first exemplary embodiment.

FIG. 4 schematically depicts a further exemplary arrangement of the organic electroluminescence device according to the first exemplary embodiment.

FIG. 5 schematically depicts a still further exemplary arrangement of the organic electroluminescence device according to the first exemplary embodiment.

FIG. 6 schematically depicts a still further exemplary arrangement of the organic electroluminescence device according to the first exemplary embodiment.

FIG. 7 schematically depicts a still further exemplary arrangement of the organic electroluminescence device according to the first exemplary embodiment.

FIG. 8 schematically depicts a still further exemplary arrangement of the organic electroluminescence device according to the first exemplary embodiment.

### DESCRIPTION OF EMBODIMENT(S)

### Definitions

Herein, a hydrogen atom includes isotope having different numbers of neutrons, specifically, protium, deuterium and tritium.

In chemical formulae herein, it is assumed that a hydrogen atom (i.e. protium, deuterium and tritium) is bonded to each of bondable positions that are not annexed with signs “R” or the like or “D” representing a deuterium.

Herein, the ring carbon atoms refer to the number of carbon atoms among atoms forming a ring of a compound (e.g., a monocyclic compound, fused-ring compound, cross-linking compound, carbon ring compound, and heterocyclic compound) in which the atoms are bonded to each other to form the ring. When the ring is substituted by a substituent(s), carbon atom(s) contained in the substituent(s) is not counted in the ring carbon atoms. Unless otherwise specified, the same applies to the “ring carbon atoms” described later. For instance, a benzene ring has 6 ring carbon atoms, a naphthalene ring has 10 ring carbon atoms, a pyridine ring has 5 ring carbon atoms, and a furan ring has 4 ring carbon atoms. Further, for instance, 9,9-diphenylfluorenyl group has 13 ring carbon atoms and 9,9′-spirobifluorenyl group has 25 ring carbon atoms.

When a benzene ring is substituted by a substituent in a form of, for instance, an alkyl group, the number of carbon atoms of the alkyl group is not counted in the number of the ring carbon atoms of the benzene ring. Accordingly, the benzene ring substituted by an alkyl group has 6 ring carbon atoms. When a naphthalene ring is substituted by a substituent in a form of, for instance, an alkyl group, the number of carbon atoms of the alkyl group is not counted in the number of the ring carbon atoms of the naphthalene ring. Accordingly, the naphthalene ring substituted by an alkyl group has 10 ring carbon atoms.

Herein, the ring atoms refer to the number of atoms forming a ring of a compound (e.g., a monocyclic compound, fused-ring compound, cross-linking compound, carbon ring compound, and heterocyclic compound) in which the atoms are bonded to each other to form the ring (e.g., monocyclic ring, fused ring, and ring assembly). Atom(s) not forming the ring (e.g., hydrogen atom(s) for saturating the valence of the atom which forms the ring) and atom(s) in a substituent by which the ring is substituted are not counted as the ring atoms. Unless otherwise specified, the same applies to the “ring atoms” described later. For instance, a pyridine ring has 6 ring atoms, a quinazoline ring has 10 ring atoms, and a furan ring has 5 ring atoms. For instance, the number of hydrogen atom(s) bonded to a pyridine ring or the number of atoms forming a substituent is not counted as the pyridine ring atoms. Accordingly, a pyridine ring bonded to a hydrogen atom(s) or a substituent(s) has 6 ring atoms. For instance, the hydrogen atom(s) bonded to carbon atom(s) of a quinazoline ring or the atoms forming a substituent are not counted as the quinazoline ring atoms. Accordingly, a quinazoline ring bonded to hydrogen atom(s) or a substituent(s) has 10 ring atoms.

Herein, “XX to YY carbon atoms” in the description of “substituted or unsubstituted ZZ group having XX to YY carbon atoms” represent carbon atoms of an unsubstituted ZZ group and do not include carbon atoms of a substituent(s) of the substituted ZZ group. Herein, “YY” is larger than “XX,” “XX” representing an integer of 1 or more and “YY” representing an integer of 2 or more.

Herein, “XX to YY atoms” in the description of “substituted or unsubstituted ZZ group having XX to YY atoms” represent atoms of an unsubstituted ZZ group and does not include atoms of a substituent(s) of the substituted ZZ group. Herein, “YY” is larger than “XX,” “XX” representing an integer of 1 or more and “YY” representing an integer of 2 or more.

Herein, an unsubstituted ZZ group refers to an “unsubstituted ZZ group” in a “substituted or unsubstituted ZZ group,” and a substituted ZZ group refers to a “substituted ZZ group” in a “substituted or unsubstituted ZZ group.”

Herein, the term “unsubstituted” used in a “substituted or unsubstituted ZZ group” means that a hydrogen atom(s) in the ZZ group is not substituted with a substituent(s). The hydrogen atom(s) in the “unsubstituted ZZ group” is protium, deuterium, or tritium.

Herein, the term “substituted” used in a “substituted or unsubstituted ZZ group” means that at least one hydrogen atom in the ZZ group is substituted with a substituent. Similarly, the term “substituted” used in a “BB group substituted by AA group” means that at least one hydrogen atom in the BB group is substituted with the AA group.

### Substituent Mentioned Herein

Substituent mentioned herein will be described below.

An “unsubstituted aryl group” mentioned herein has, unless otherwise specified herein, 6 to 50, preferably 6 to 30, more preferably 6 to 18 ring carbon atoms.

An “unsubstituted heterocyclic group” mentioned herein has, unless otherwise specified herein, 5 to 50, preferably 5 to 30, more preferably 5 to 18 ring atoms.

An “unsubstituted alkyl group” mentioned herein has, unless otherwise specified herein, 1 to 50, preferably 1 to 20, more preferably 1 to 6 carbon atoms.

An “unsubstituted alkenyl group” mentioned herein has, unless otherwise specified herein, 2 to 50, preferably 2 to 20, more preferably 2 to 6 carbon atoms.

An “unsubstituted alkynyl group” mentioned herein has, unless otherwise specified herein, 2 to 50, preferably 2 to 20, more preferably 2 to 6 carbon atoms.

An “unsubstituted cycloalkyl group” mentioned herein has, unless otherwise specified herein, 3 to 50, preferably 3 to 20, more preferably 3 to 6 ring carbon atoms.

An “unsubstituted arylene group” mentioned herein has, unless otherwise specified herein, 6 to 50, preferably 6 to 30, more preferably 6 to 18 ring carbon atoms.

An “unsubstituted divalent heterocyclic group” mentioned herein has, unless otherwise specified herein, 5 to 50, preferably 5 to 30, more preferably 5 to 18 ring atoms.

An “unsubstituted alkylene group” mentioned herein has, unless otherwise specified herein, 1 to 50, preferably 1 to 20, more preferably 1 to 6 carbon atoms.

### Substituted or Unsubstituted Aryl Group

Specific examples (specific example group G1) of the “substituted or unsubstituted aryl group” mentioned herein include unsubstituted aryl groups (specific example group G1A) below and substituted aryl groups (specific example group G11B). (Herein, an unsubstituted aryl group refers to an “unsubstituted aryl group” in a “substituted or unsubstituted aryl group”, and a substituted aryl group refers to a “substituted aryl group” in a “substituted or unsubstituted aryl group.”) A simply termed “aryl group” herein includes both of an “unsubstituted aryl group” and a “substituted aryl group”.

The “substituted aryl group” refers to a group derived by substituting at least one hydrogen atom in an “unsubstituted aryl group” with a substituent. Examples of the “substituted aryl group” include a group derived by substituting at least one hydrogen atom in the “unsubstituted aryl group” in the specific example group G1A below with a substituent, and examples of the substituted aryl group in the specific example group G1B below. It should be noted that the examples of the “unsubstituted aryl group” and the “substituted aryl group” mentioned herein are merely exemplary, and the “substituted aryl group” mentioned herein includes a group derived by further substituting a hydrogen atom bonded to a carbon atom of a skeleton of a “substituted aryl group” in the specific example group G1B below, and a group derived by further substituting a hydrogen atom of a substituent of the “substituted aryl group” in the specific example group G1B below.

### Unsubstituted Aryl Group (Specific Example Group G1A):

a phenyl group, p-biphenyl group, m-biphenyl group, o-biphenyl group, p-terphenyl-4-yl group, p-terphenyl-3-yl group, p-terphenyl-2-yl group, m-terphenyl-4-yl group, m-terphenyl-3-yl group, m-terphenyl-2-yl group, o-terphenyl-4-yl group, o-terphenyl-3-yl group, o-terphenyl-2-yl group, 1-naphthyl group, 2-naphthyl group, anthryl group, benzanthryl group, phenanthryl group, benzophenanthryl group, phenalenyl group, pyrenyl group, chrysenyl group, benzochrysenyl group, triphenylenyl group, benzotriphenylenyl group, tetracenyl group, pentacenyl group, fluorenyl group, 9,9′-spirobifluorenyl group, benzofluorenyl group, dibenzofluorenyl group, fluoranthenyl group, benzofluoranthenyl group, perylenyl group, and monovalent aryl group derived by removing one hydrogen atom from cyclic structures represented by formulae (TEMP-1) to (TEMP-15) below.

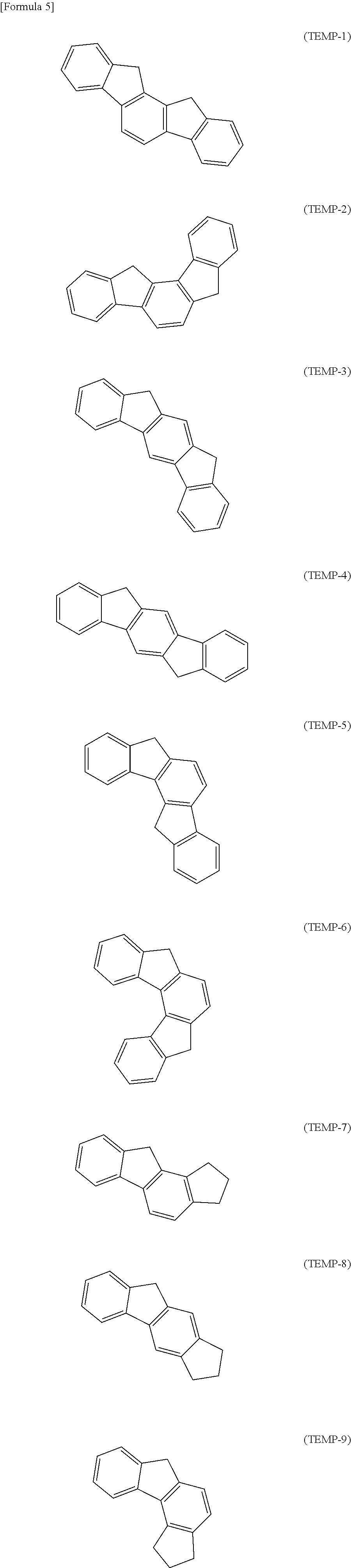

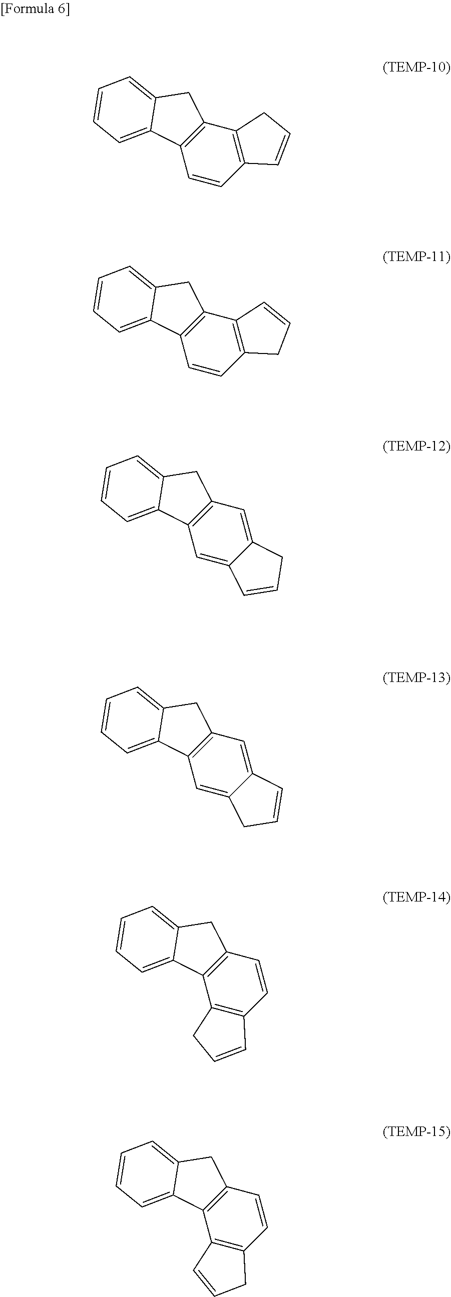

### Substituted Aryl Group (Specific Example Group G1B):

an o-tolyl group, m-tolyl group, p-tolyl group, para-xylyl group, meta-xylyl group, ortho-xylyl group, para-isopropylphenyl group, meta-isopropylphenyl group, ortho-isopropylphenyl group, para-t-butylphenyl group, meta-t-butylphenyl group, ortho-t-butylphenyl group, 3,4,5-trimethylphenyl group, 9,9-dimethylfluorenyl group, 9,9-diphenylfluorenyl group, 9,9-bis(4-methylphenyl)fluorenyl group, 9,9-bis(4-isopropylphenyl)fluorenyl group, 9,9-bis(4-t-butylphenyl)fluorenyl group, cyanophenyl group, triphenylsilylphenyl group, trimethylsilylphenyl group, phenylnaphthyl group, naphthylphenyl group, and group derived by substituting at least one hydrogen atom of a monovalent group derived from one of the cyclic structures represented by the formulae (TEMP-1) to (TEMP-15) with a substituent.

### Substituted or Unsubstituted Heterocyclic Group

The “heterocyclic group” mentioned herein refers to a cyclic group having at least one hetero atom in the ring atoms. Specific examples of the hetero atom include a nitrogen atom, oxygen atom, sulfur atom, silicon atom, phosphorus atom, and boron atom.

The “heterocyclic group” mentioned herein is a monocyclic group or a fused-ring group.

The “heterocyclic group” mentioned herein is an aromatic heterocyclic group or a non-aromatic heterocyclic group.

Specific examples (specific example group G2) of the “substituted or unsubstituted heterocyclic group” mentioned herein include unsubstituted heterocyclic groups (specific example group G2A) and substituted heterocyclic groups (specific example group G2B). (Herein, an unsubstituted heterocyclic group refers to an “unsubstituted heterocyclic group” in a “substituted or unsubstituted heterocyclic group,” and a substituted heterocyclic group refers to a “substituted heterocyclic group” in a “substituted or unsubstituted heterocyclic group.”) A simply termed “heterocyclic group” herein includes both of an “unsubstituted heterocyclic group” and a “substituted heterocyclic group.”

The “substituted heterocyclic group” refers to a group derived by substituting at least one hydrogen atom in an “unsubstituted heterocyclic group” with a substituent. Specific examples of the “substituted heterocyclic group” include a group derived by substituting at least one hydrogen atom in the “unsubstituted heterocyclic group” in the specific example group G2A below with a substituent, and examples of the substituted heterocyclic group in the specific example group G2B below. It should be noted that the examples of the “unsubstituted heterocyclic group” and the “substituted heterocyclic group” mentioned herein are merely exemplary, and the “substituted heterocyclic group” mentioned herein includes a group derived by further substituting a hydrogen atom bonded to a ring atom of a skeleton of a “substituted heterocyclic group” in the specific example group G2B below, and a group derived by further substituting a hydrogen atom of a substituent of the “substituted heterocyclic group” in the specific example group G2B below.

The specific example group G2A includes, for instance, unsubstituted heterocyclic groups including a nitrogen atom (specific example group G2A1) below, unsubstituted heterocyclic groups including an oxygen atom (specific example group G2A2) below, unsubstituted heterocyclic groups including a sulfur atom (specific example group G2A3) below, and monovalent heterocyclic groups (specific example group G2A4) derived by removing a hydrogen atom from cyclic structures represented by formulae (TEMP-16) to (TEMP-33) below.

The specific example group G2B includes, for instance, substituted heterocyclic groups including a nitrogen atom (specific example group G2B1) below, substituted heterocyclic groups including an oxygen atom (specific example group G2B2) below, substituted heterocyclic groups including a sulfur atom (specific example group G2B3) below, and groups derived by substituting at least one hydrogen atom of the monovalent heterocyclic groups (specific example group G2B4) derived from the cyclic structures represented by formulae (TEMP-16) to (TEMP-33) below.

### Unsubstituted Heterocyclic Groups Including Nitrogen Atom (Specific Example Group G2A1):

a pyrrolyl group, imidazolyl group, pyrazolyl group, triazolyl group, tetrazolyl group, oxazolyl group, isoxazolyl group, oxadiazolyl group, thiazolyl group, isothiazolyl group, thiadiazolyl group, pyridyl group, pyridazynyl group, pyrimidinyl group, pyrazinyl group, triazinyl group, indolyl group, isoindolyl group, indolizinyl group, quinolizinyl group, quinolyl group, isoquinolyl group, cinnolyl group, phthalazinyl group, quinazolinyl group, quinoxalinyl group, benzimidazolyl group, indazolyl group, phenanthrolinyl group, phenanthridinyl group, acridinyl group, phenazinyl group, carbazolyl group, benzocarbazolyl group, morpholino group, phenoxazinyl group, phenothiazinyl group, azacarbazolyl group, and diazacarbazolyl group.

### Unsubstituted Heterocyclic Groups Including Oxygen Atom (Specific Example Group G2A2):

a furyl group, oxazolyl group, isoxazolyl group, oxadiazolyl group, xanthenyl group, benzofuranyl group, isobenzofuranyl group, dibenzofuranyl group, naphthobenzofuranyl group, benzoxazolyl group, benzisoxazolyl group, phenoxazinyl group, morpholino group, dinaphthofuranyl group, azadibenzofuranyl group, diazadibenzofuranyl group, azanaphthobenzofuranyl group, and diazanaphthobenzofuranyl group.

### Unsubstituted Heterocyclic Groups Including Sulfur Atom (Specific Example Group G2A3):

a thienyl group, thiazolyl group, isothiazolyl group, thiadiazolyl group, benzothiophenyl group (benzothienyl group), isobenzothiophenyl group (isobenzothienyl group), dibenzothiophenyl group (dibenzothienyl group), naphthobenzothiophenyl group (nahthobenzothienyl group), benzothiazolyl group, benzisothiazolyl group, phenothiazinyl group, dinaphthothiophenyl group (dinaphthothienyl group), azadibenzothiophenyl group (azadibenzothienyl group), diazadibenzothiophenyl group (diazadibenzothienyl group), azanaphthobenzothiophenyl group (azanaphthobenzothienyl group), and diazanaphthobenzothiophenyl group (diazanaphthobenzothienyl group).

Monovalent Heterocyclic Groups Derived by Removing One Hydrogen Atom from Cyclic Structures Represented by Formulae (TEMP-16) to (TEMP-33) (Specific Example Group G2A4):

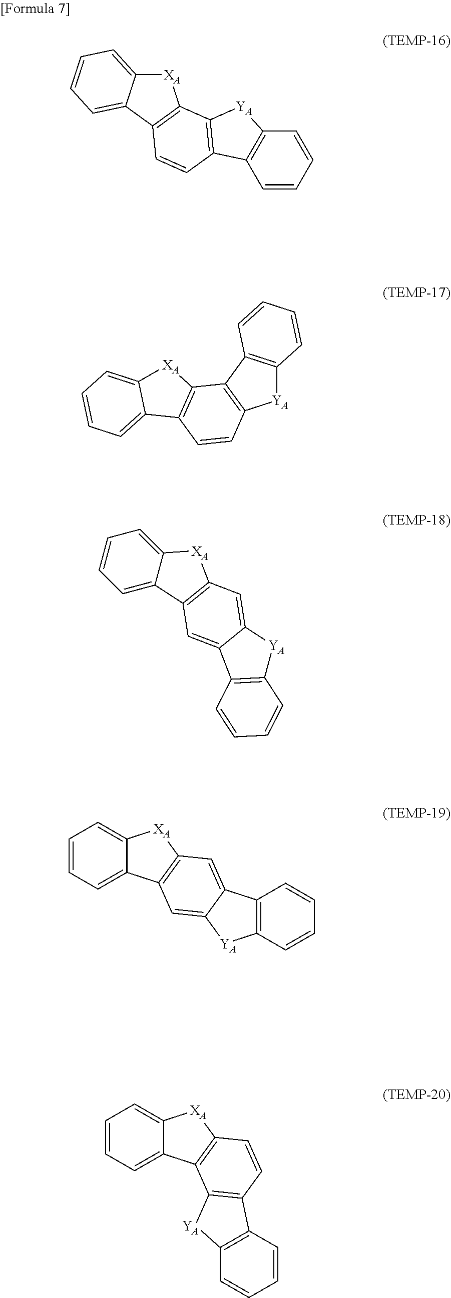

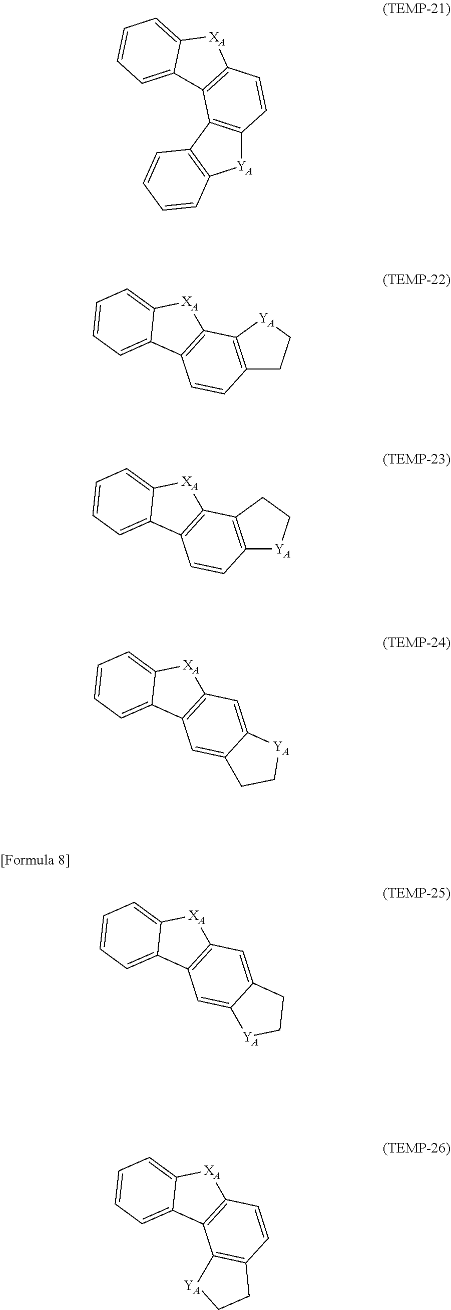

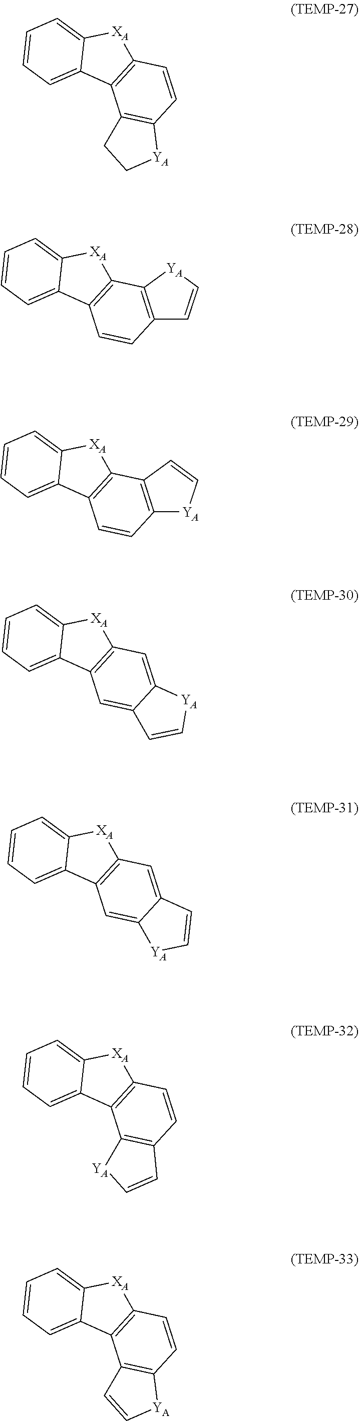

In the formulae (TEMP-16) to (TEMP-33), X_{A} and Y_{A} are each independently an oxygen atom, a sulfur atom, NH or CH₂, with a proviso that at least one of X_{A} or Y_{A} is an oxygen atom, a sulfur atom, or NH.

When at least one of X_{A} or Y_{A} in the formulae (TEMP-16) to (TEMP-33) is NH or CH₂, the monovalent heterocyclic groups derived from the cyclic structures represented by the formulae (TEMP-16) to (TEMP-33) include a monovalent group derived by removing one hydrogen atom from NH or CH₂.

### Substituted Heterocyclic Groups Including Nitrogen Atom (Specific Example Group G2B1):

(9-phenyl)carbazolyl group, (9-biphenylyl)carbazolyl group, (9-phenyl)phenylcarbazolyl group, (9-naphthyl)carbazolyl group, diphenylcarbazole-9-yl group, phenylcarbazole-9-yl group, methylbenzimidazolyl group, ethylbenzimidazolyl group, phenyltriazinyl group, biphenylyltriazinyl group, diphenyltriazinyl group, phenylquinazolinyl group, and biphenylquinazolinyl group.

### Substituted Heterocyclic Groups Including Oxygen Atom (Specific Example Group G2B2):

a phenyldibenzofuranyl group, methyldibenzofuranyl group, t-butyldibenzofuranyl group, and monovalent residue of spiro[9H-xanthene-9,9′-[9H]fluorene].

### Substituted Heterocyclic Groups Including Sulfur Atom (Specific Example Group G2B3):

a phenyldibenzothiophenyl group, methyldibenzothiophenyl group, t-butyldibenzothiophenyl group, and monovalent residue of spiro[9H-thioxanthene-9,9′-[9H]fluorene].

Groups Obtained by Substituting at Least One Hydrogen Atom of Monovalent Heterocyclic Group Derived from Cyclic Structures Represented by Formulae (TEMP-16) to (TEMP-33) with Substituent (Specific Example Group G2B4):

The “at least one hydrogen atom of a monovalent heterocyclic group” means at least one hydrogen atom selected from a hydrogen atom bonded to a ring carbon atom of the monovalent heterocyclic group, a hydrogen atom bonded to a nitrogen atom of at least one of X_{A} or Y_{A} in a form of NH, and a hydrogen atom of one of X_{A} and Y_{A} in a form of a methylene group (CH₂).

### Substituted or Unsubstituted Alkyl Group

Specific examples (specific example group G3) of the “substituted or unsubstituted alkyl group” mentioned herein include unsubstituted alkyl groups (specific example group G3A) and substituted alkyl groups (specific example group G3B) below. (Herein, an unsubstituted alkyl group refers to an “unsubstituted alkyl group” in a “substituted or unsubstituted alkyl group,” and a substituted alkyl group refers to a “substituted alkyl group” in a “substituted or unsubstituted alkyl group.”) A simply termed “alkyl group” herein includes both of an “unsubstituted alkyl group” and a “substituted alkyl group”.

The “substituted alkyl group” refers to a group derived by substituting at least one hydrogen atom in an “unsubstituted alkyl group” with a substituent. Specific examples of the “substituted alkyl group” include a group derived by substituting at least one hydrogen atom of an “unsubstituted alkyl group” (specific example group G3A) below with a substituent, and examples of the substituted alkyl group (specific example group G3B) below. Herein, the alkyl group for the “unsubstituted alkyl group” refers to a chain alkyl group. Accordingly, the “unsubstituted alkyl group” include linear “unsubstituted alkyl group” and branched “unsubstituted alkyl group.” It should be noted that the examples of the “unsubstituted alkyl group” and the “substituted alkyl group” mentioned herein are merely exemplary, and the “substituted alkyl group” mentioned herein includes a group derived by further substituting a hydrogen atom of a skeleton of the “substituted alkyl group” in the specific example group G3B, and a group derived by further substituting a hydrogen atom of a substituent of the “substituted alkyl group” in the specific example group G3B.

### Unsubstituted Alkyl Group (Specific Example Group G3A):

a methyl group, ethyl group, n-propyl group, isopropyl group, n-butyl group, isobutyl group, s-butyl group, and t-butyl group.

### Substituted Alkyl Group (Specific Example Group G3B):

a heptafluoropropyl group (including isomer thereof), pentafluoroethyl group, 2,2,2-trifluoroethyl group, and trifluoromethyl group.

### Substituted or Unsubstituted Alkenyl Group

Specific examples (specific example group G4) of the “substituted or unsubstituted alkenyl group” mentioned herein include unsubstituted alkenyl groups (specific example group G4A) and substituted alkenyl groups (specific example group G4B). (Herein, an unsubstituted alkenyl group refers to an “unsubstituted alkenyl group” in a “substituted or unsubstituted alkenyl group,” and a substituted alkenyl group refers to a “substituted alkenyl group” in a “substituted or unsubstituted alkenyl group.”) A simply termed “alkenyl group” herein includes both of an “unsubstituted alkenyl group” and a “substituted alkenyl group”.

The “substituted alkenyl group” refers to a group derived by substituting at least one hydrogen atom in an “unsubstituted alkenyl group” with a substituent. Specific examples of the “substituted alkenyl group” include an “unsubstituted alkenyl group” (specific example group G4A) substituted by a substituent, and examples of the substituted alkenyl group (specific example group G4B) below. It should be noted that the examples of the “unsubstituted alkenyl group” and the “substituted alkenyl group” mentioned herein are merely exemplary, and the “substituted alkenyl group” mentioned herein includes a group derived by further substituting a hydrogen atom of a skeleton of the “substituted alkenyl group” in the specific example group G4B with a substituent, and a group derived by further substituting a hydrogen atom of a substituent of the “substituted alkenyl group” in the specific example group G4B with a substituent.

### Unsubstituted Alkenyl Group (Specific Example Group G4A):

a vinyl group, allyl group, 1-butenyl group, 2-butenyl group, and 3-butenyl group.

### Substituted Alkenyl Group (Specific Example Group G4B):

a 1,3-butanedienyl group, 1-methylvinyl group, 1-methylallyl group, 1,1-dimethylallyl group, 2-methylallyl group, and 1,2-dimethylallyl group.

### Substituted or Unsubstituted Alkynyl Group

Specific examples (specific example group G5) of the “substituted or unsubstituted alkynyl group” mentioned herein include unsubstituted alkynyl groups (specific example group G5A) below. (Herein, an unsubstituted alkynyl group refers to an “unsubstituted alkynyl group” in a “substituted or unsubstituted alkynyl group.”) A simply termed “alkynyl group” herein includes both of “unsubstituted alkynyl group” and “substituted alkynyl group”.

The “substituted alkynyl group” refers to a group derived by substituting at least one hydrogen atom in an “unsubstituted alkynyl group” with a substituent. Specific examples of the “substituted alkynyl group” include a group derived by substituting at least one hydrogen atom of the “unsubstituted alkynyl group” (specific example group G5A) below with a substituent.

Unsubstituted Alkynyl Group (Specific Example Group G5A): ethynyl group

### Substituted or Unsubstituted Cycloalkyl Group

Specific examples (specific example group G6) of the “substituted or unsubstituted cycloalkyl group” mentioned herein include unsubstituted cycloalkyl groups (specific example group G6A) and substituted cycloalkyl groups (specific example group G6B). (Herein, an unsubstituted cycloalkyl group refers to an “unsubstituted cycloalkyl group” in a “substituted or unsubstituted cycloalkyl group,” and a substituted cycloalkyl group refers to a “substituted cycloalkyl group” in a “substituted or unsubstituted cycloalkyl group.”) A simply termed “cycloalkyl group” herein includes both of “unsubstituted cycloalkyl group” and “substituted cycloalkyl group”.

The “substituted cycloalkyl group” refers to a group derived by substituting at least one hydrogen atom of an “unsubstituted cycloalkyl group” with a substituent. Specific examples of the “substituted cycloalkyl group” include a group derived by substituting at least one hydrogen atom of the “unsubstituted cycloalkyl group” (specific example group G6A) below with a substituent, and examples of the substituted cycloalkyl group (specific example group G6B) below. It should be noted that the examples of the “unsubstituted cycloalkyl group” and the “substituted cycloalkyl group” mentioned herein are merely exemplary, and the “substituted cycloalkyl group” mentioned herein includes a group derived by substituting at least one hydrogen atom bonded to a carbon atom of a skeleton of the “substituted cycloalkyl group” in the specific example group G6B with a substituent, and a group derived by further substituting a hydrogen atom of a substituent of the “substituted cycloalkyl group” in the specific example group G6B with a substituent.

### Unsubstituted Cycloalkyl Group (Specific Example Group G6A):

a cyclopropyl group, cyclobutyl group, cyclopentyl group, cyclohexyl group, 1-adamantyl group, 2-adamantyl group, 1-norbornyl group, and 2-norbornyl group.

### Substituted Cycloalkyl Group (Specific Example Group G6B):

a 4-methylcyclohexyl group.

Group Represented by —Si(R₉₀₁)(R₉₀₂)(R₉₀₃)

Specific examples (specific example group G7) of the group represented herein by —Si(R₉₀₁)(R₉₀₂)(R₉₀₃) include: —Si(G1)(G1)(G1); —Si(G1)(G2)(G2); —Si(G1)(G1)(G2); —Si(G2)(G2)(G2); —Si(G3)(G3)(G3); and —Si(G6)(G6)(G6);
where:
G1 represents a “substituted or unsubstituted aryl group” in the specific example group G1;
G2 represents a “substituted or unsubstituted heterocyclic group” in the specific example group G2;
G3 represents a “substituted or unsubstituted alkyl group” in the specific example group G3;
G6 represents a “substituted or unsubstituted cycloalkyl group” in the specific example group G6;
a plurality of G1 in —Si(G1)(G1)(G1) are mutually the same or different;
a plurality of G2 in —Si(G1)(G2)(G2) are mutually the same or different;
a plurality of G1 in —Si(G1)(G1)(G2) are mutually the same or different;
a plurality of G2 in —Si(G2)(G2)(G2) are mutually the same or different;
a plurality of G3 in —Si(G3)(G3)(G3) are mutually the same or different; and
a plurality of G6 in —Si(G6)(G6)(G6) are mutually the same or different.

### Group Represented by —O—(R₉₀₄)

Specific examples (specific example group G8) of a group represented by —O—(R₉₀₄) herein include: —O(G1); —O(G2); —O(G3); and —O(G6); where:
G1 represents a “substituted or unsubstituted aryl group” in the specific example group G1;
G2 represents a “substituted or unsubstituted heterocyclic group” in the specific example group G2;
G3 represents a “substituted or unsubstituted alkyl group” in the specific example group G3; and
G6 represents a “substituted or unsubstituted cycloalkyl group” in the specific example group G6.

### Group Represented by —S—(R₉₀₅)

Specific examples (specific example group G9) of a group represented herein by —S—(R₉₀₅) include: —S(G1); —S(G2); —S(G3); and —S(G6);
where:
G1 represents a “substituted or unsubstituted aryl group” in the specific example group G1;
G2 represents a “substituted or unsubstituted heterocyclic group” in the specific example group G2;
G3 represents a “substituted or unsubstituted alkyl group” in the specific example group G3; and
G6 represents a “substituted or unsubstituted cycloalkyl group” in the specific example group G6.
   Group Represented by —N(R₉₀₆)(R₉₀₇)

Specific examples (specific example group G10) of a group represented herein by —N(R₉₀₆)(R₉₀₇) include: —N(G1)(G1); —N(G2)(G2); —N(G1)(G2); —N(G3)(G3); and —N(G6)(G6),
where:
G1 represents a “substituted or unsubstituted aryl group” in the specific example group G1;
G2 represents a “substituted or unsubstituted heterocyclic group” in the specific example group G2;
G3 represents a “substituted or unsubstituted alkyl group” in the specific example group G3;
G6 represents a “substituted or unsubstituted cycloalkyl group” in the specific example group G6;
a plurality of G1 in —N(G1)(G1) are mutually the same or different;
a plurality of G2 in —N(G2)(G2) are mutually the same or different;
a plurality of G3 in —N(G3)(G3) are mutually the same or different; and
a plurality of G6 in —N(G6)(G6) are mutually the same or different.

### Halogen Atom

Specific examples (specific example group G11) of “halogen atom” mentioned herein include a fluorine atom, chlorine atom, bromine atom, and iodine atom.

### Substituted or Unsubstituted Fluoroalkyl Group

The “substituted or unsubstituted fluoroalkyl group” mentioned herein refers to a group derived by substituting at least one hydrogen atom bonded to at least one of carbon atoms forming an alkyl group in the “substituted or unsubstituted alkyl group” with a fluorine atom, and also includes a group (perfluoro group) derived by substituting all of hydrogen atoms bonded to carbon atoms forming the alkyl group in the “substituted or unsubstituted alkyl group” with fluorine atoms. An “unsubstituted fluoroalkyl group” has, unless otherwise specified herein, 1 to 50, preferably 1 to 30, more preferably 1 to 18 carbon atoms. The “substituted fluoroalkyl group” refers to a group derived by substituting at least one hydrogen atom in a “fluoroalkyl group” with a substituent. It should be noted that the examples of the “substituted fluoroalkyl group” mentioned herein include a group derived by further substituting at least one hydrogen atom bonded to a carbon atom of an alkyl chain of a “substituted fluoroalkyl group” with a substituent, and a group derived by further substituting at least one hydrogen atom of a substituent of the “substituted fluoroalkyl group” with a substituent. Specific examples of the “unsubstituted fluoroalkyl group” include a group derived by substituting at least one hydrogen atom of the “alkyl group” (specific example group G3) with a fluorine atom.

### Substituted or Unsubstituted Haloalkyl Group

The “substituted or unsubstituted haloalkyl group” mentioned herein refers to a group derived by substituting at least one hydrogen atom bonded to carbon atoms forming the alkyl group in the “substituted or unsubstituted alkyl group” with a halogen atom, and also includes a group derived by substituting all hydrogen atoms bonded to carbon atoms forming the alkyl group in the “substituted or unsubstituted alkyl group” with halogen atoms. An “unsubstituted haloalkyl group” has, unless otherwise specified herein, 1 to 50, preferably 1 to 30, and more preferably 1 to 18 carbon atoms. The “substituted haloalkyl group” refers to a group derived by substituting at least one hydrogen atom in a “haloalkyl group” with a substituent. It should be noted that the examples of the “substituted haloalkyl group” mentioned herein include a group derived by further substituting at least one hydrogen atom bonded to a carbon atom of an alkyl chain of a “substituted haloalkyl group” with a substituent, and a group derived by further substituting at least one hydrogen atom of a substituent of the “substituted haloalkyl group” with a substituent. Specific examples of the “unsubstituted haloalkyl group” include a group derived by substituting at least one hydrogen atom of the “alkyl group” (specific example group G3) with a halogen atom. The haloalkyl group is sometimes referred to as a halogenated alkyl group.

### Substituted or Unsubstituted Alkoxy Group

Specific examples of a “substituted or unsubstituted alkoxy group” mentioned herein include a group represented by —O(G3), G3 being the “substituted or unsubstituted alkyl group” in the specific example group G3. An “unsubstituted alkoxy group” has, unless otherwise specified herein, 1 to 50, preferably 1 to 30, more preferably 1 to 18 carbon atoms.

### Substituted or Unsubstituted Alkylthio Group

Specific examples of a “substituted or unsubstituted alkylthio group” mentioned herein include a group represented by —S(G3), G3 being the “substituted or unsubstituted alkyl group” in the specific example group G3. An “unsubstituted alkylthio group” has, unless otherwise specified herein, 1 to 50, preferably 1 to 30, more preferably 1 to 18 carbon atoms.

### Substituted or Unsubstituted Aryloxy Group

Specific examples of a “substituted or unsubstituted aryloxy group” mentioned herein include a group represented by —O(G1), G1 being the “substituted or unsubstituted aryl group” in the specific example group G1. An “unsubstituted aryloxy group” has, unless otherwise specified herein, 6 to 50, preferably 6 to 30, more preferably 6 to 18 ring carbon atoms.

### Substituted or Unsubstituted Arylthio Group

Specific examples of a “substituted or unsubstituted arylthio group” mentioned herein include a group represented by —S(G1), G1 being the “substituted or unsubstituted aryl group” in the specific example group G1. An “unsubstituted arylthio group” has, unless otherwise specified herein, 6 to 50, preferably 6 to 30, more preferably 6 to 18 ring carbon atoms.

### Substituted or Unsubstituted Trialkylsilyl Group

Specific examples of a “trialkylsilyl group” mentioned herein include a group represented by —Si(G3)(G3)(G3), G3 being the “substituted or unsubstituted alkyl group” in the specific example group G3. The plurality of G3 in —Si(G3)(G3)(G3) are mutually the same or different. Each of the alkyl groups in the “trialkylsilyl group” has, unless otherwise specified herein, 1 to 50, preferably 1 to 20, more preferably 1 to 6 carbon atoms.

### Substituted or Unsubstituted Aralkyl Group

Specific examples of a “substituted or unsubstituted aralkyl group” mentioned herein include a group represented by -(G3)-(G1), G3 being the “substituted or unsubstituted alkyl group” in the specific example group G3, G1 being the “substituted or unsubstituted aryl group” in the specific example group G1. Accordingly, the “aralkyl group” is a group derived by substituting a hydrogen atom of the “alkyl group” with a substituent in a form of the “aryl group,” which is an example of the “substituted alkyl group.” An “unsubstituted aralkyl group,” which is an “unsubstituted alkyl group” substituted by an “unsubstituted aryl group,” has, unless otherwise specified herein, 7 to 50 carbon atoms, preferably 7 to 30 carbon atoms, more preferably 7 to 18 carbon atoms.

Specific examples of the “substituted or unsubstituted aralkyl group” include a benzyl group, 1-phenylethyl group, 2-phenylethyl group, 1-phenylisopropyl group, 2-phenylisopropyl group, phenyl-t-butyl group, α-naphthylmethyl group, 1-α-naphthylethyl group, 2-α-naphthylethyl group, 1-α-naphthylisopropyl group, 2-α-naphthylisopropyl group, β-naphthylmethyl group, 1-β-naphthylethyl group, 2-β-naphthylethyl group, 1-β-naphthylisopropyl group, and 2-β-naphthylisopropyl group.

Preferable examples of the substituted or unsubstituted aryl group mentioned herein include, unless otherwise specified herein, a phenyl group, p-biphenyl group, m-biphenyl group, o-biphenyl group, p-terphenyl-4-yl group, p-terphenyl-3-yl group, p-terphenyl-2-yl group, m-terphenyl-4-yl group, m-terphenyl-3-yl group, m-terphenyl-2-yl group, o-terphenyl-4-yl group, o-terphenyl-3-yl group, o-terphenyl-2-yl group, 1-naphthyl group, 2-naphthyl group, anthryl group, phenanthryl group, pyrenyl group, chrysenyl group, triphenylenyl group, fluorenyl group, 9,9′-spirobifluorenyl group, 9,9-dimethylfluorenyl group, and 9,9-diphenylfluorenyl group.

Preferable examples of the substituted or unsubstituted heterocyclic group mentioned herein include, unless otherwise specified herein, a pyridyl group, pyrimidinyl group, triazinyl group, quinolyl group, isoquinolyl group, quinazolinyl group, benzimidazolyl group, phenanthrolinyl group, carbazolyl group (1-carbazolyl group, 2-carbazolyl group, 3-carbazolyl group, 4-carbazolyl group, or 9-carbazolyl group), benzocarbazolyl group, azacarbazolyl group, diazacarbazolyl group, dibenzofuranyl group, naphthobenzofuranyl group, azadibenzofuranyl group, diazadibenzofuranyl group, dibenzothiophenyl group, naphthobenzothiophenyl group, azadibenzothiophenyl group, diazadibenzothiophenyl group, (9-phenyl)carbazolyl group ((9-phenyl)carbazole-1-yl group, (9-phenyl)carbazole-2-yl group, (9-phenyl)carbazole-3-yl group, or (9-phenyl)carbazole-4-yl group), (9-biphenylyl)carbazolyl group, (9-phenyl)phenylcarbazolyl group, diphenylcarbazole-9-yl group, phenylcarbazole-9-yl group, phenyltriazinyl group, biphenylyltriazinyl group, diphenyltriazinyl group, phenyldibenzofuranyl group, and phenyldibenzothiophenyl group.

The carbazolyl group mentioned herein is, unless otherwise specified herein, specifically a group represented by one of formulae below.

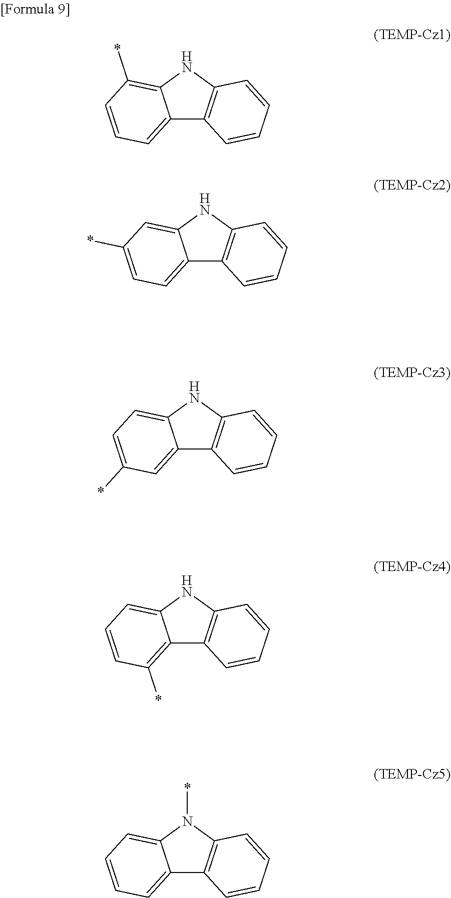 [SMILES: *c1cccc2c1[nH]c1ccccc12 ; *c1ccc2c(c1)[nH]c1ccccc12 ; *c1ccc2[nH]c3ccccc3c2c1 ; *c1cccc2[nH]c3ccccc3c12 ; *n1c2ccccc2c2ccccc21]

The (9-phenyl)carbazolyl group mentioned herein is, unless otherwise specified herein, specifically a group represented by one of formulae below.

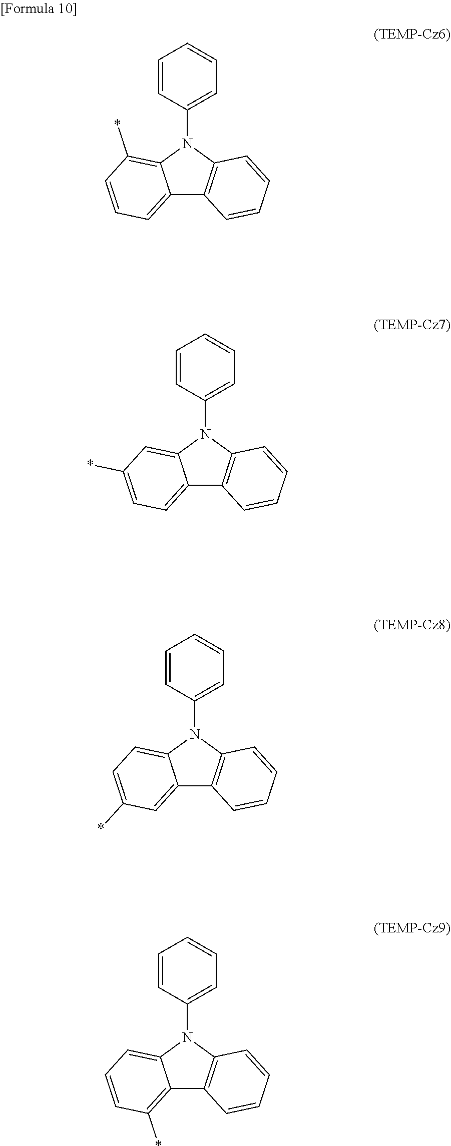 [SMILES: *c1cccc2c3ccccc3n(-c3ccccc3)c12 ; *c1ccc2c3ccccc3n(-c3ccccc3)c2c1 ; *c1ccc2c(c1)c1ccccc1n2-c1ccccc1 ; *c1cccc2c1c1ccccc1n2-c1ccccc1]

In the formulae (TEMP-Cz1) to (TEMP-Cz9), * represents a bonding position.

The dibenzofuranyl group and dibenzothiophenyl group mentioned herein are, unless otherwise specified herein, each specifically represented by one of formulae below.

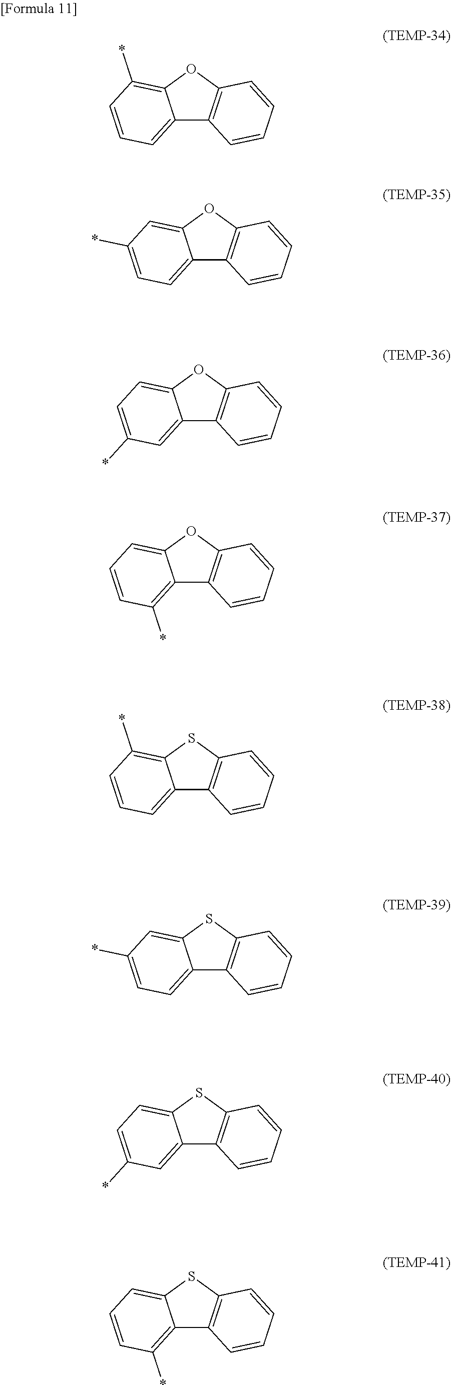

In the formulae (TEMP-34) to (TEMP-41), * represents a bonding position.

Preferable examples of the substituted or unsubstituted alkyl group mentioned herein include, unless otherwise specified herein, a methyl group, ethyl group, propyl group, isopropyl group, n-butyl group, isobutyl group, and t-butyl group.

### Substituted or Unsubstituted Arylene Group

The “substituted or unsubstituted arylene group” mentioned herein is, unless otherwise specified herein, a divalent group derived by removing one hydrogen atom on an aryl ring of the “substituted or unsubstituted aryl group.” Specific examples of the “substituted or unsubstituted arylene group” (specific example group G12) include a divalent group derived by removing one hydrogen atom on an aryl ring of the “substituted or unsubstituted aryl group” in the specific example group G1.

### Substituted or Unsubstituted Divalent Heterocyclic Group

The “substituted or unsubstituted divalent heterocyclic group” mentioned herein is, unless otherwise specified herein, a divalent group derived by removing one hydrogen atom on a heterocycle of the “substituted or unsubstituted heterocyclic group.” Specific examples of the “substituted or unsubstituted divalent heterocyclic group” (specific example group G13) include a divalent group derived by removing one hydrogen atom on a heterocyclic ring of the “substituted or unsubstituted heterocyclic group” in the specific example group G2.

### Substituted or Unsubstituted Alkylene Group

The “substituted or unsubstituted alkylene group” mentioned herein is, unless otherwise specified herein, a divalent group derived by removing one hydrogen atom on an alkyl chain of the “substituted or unsubstituted alkyl group.” Specific examples of the “substituted or unsubstituted alkylene group” (specific example group G14) include a divalent group derived by removing one hydrogen atom on an alkyl chain of the “substituted or unsubstituted alkyl group” in the specific example group G3.

The substituted or unsubstituted arylene group mentioned herein is, unless otherwise specified herein, preferably any one of groups represented by formulae (TEMP-42) to (TEMP-68) below.

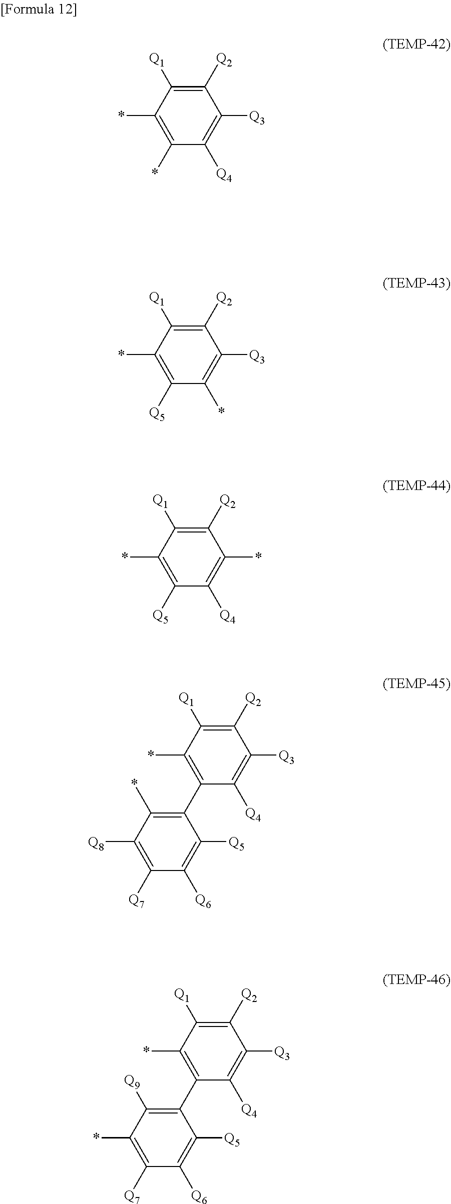

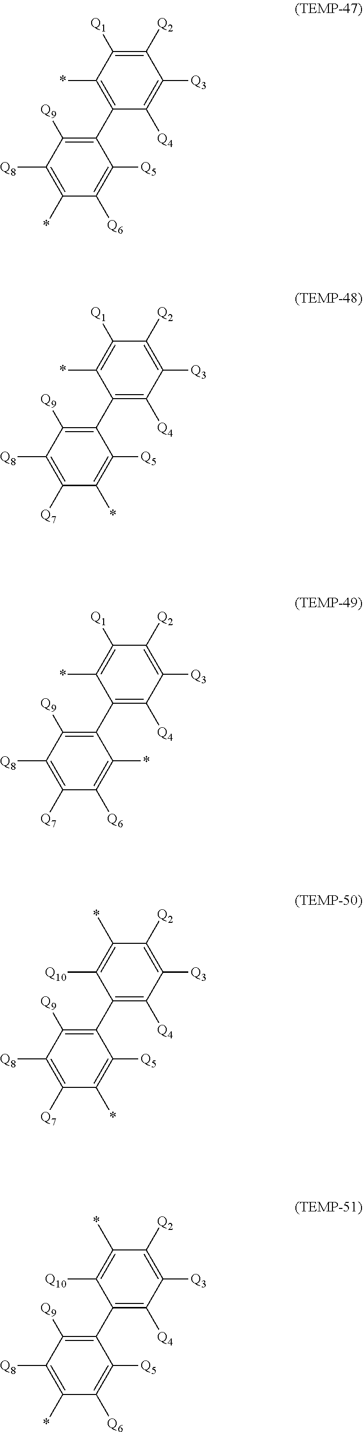

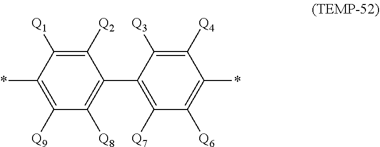

In the formulae (TEMP-42) to (TEMP-52), Q₁ to Q₁₀ are each independently a hydrogen atom or a substituent.

In the formulae (TEMP-42) to (TEMP-52), * represents a bonding position.

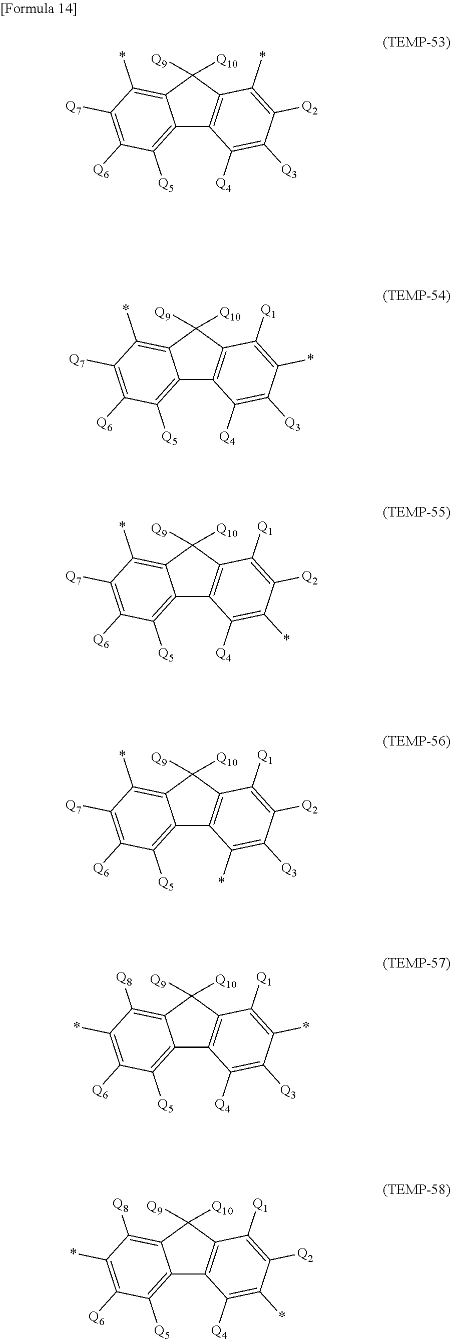

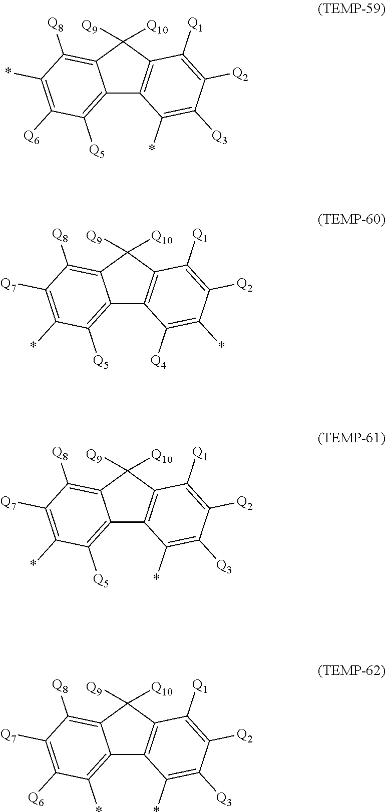

In the formulae (TEMP-53) to (TEMP-62), Q₁ to Q₁₀ are each independently a hydrogen atom or a substituent.

In the formulae, Q₉ and Q₁₀ may be mutually bonded through a single bond to form a ring.

In the formulae (TEMP-53) to (TEMP-62), * represents a bonding position.

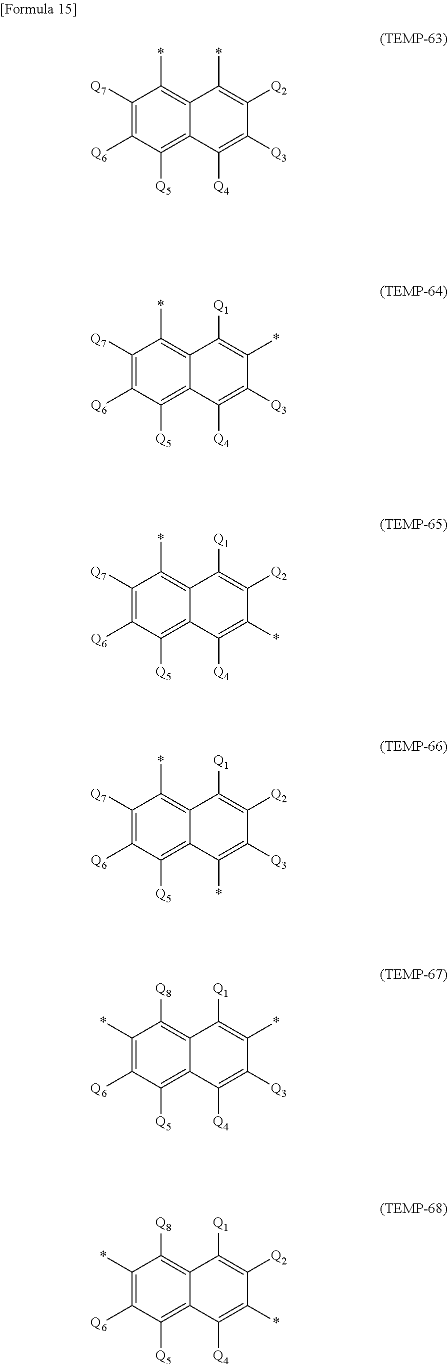

In the formulae (TEMP-63) to (TEMP-68), Q₁ to Q₈ are each independently a hydrogen atom or a substituent.

In the formulae (TEMP-63) to (TEMP-68), * represents a bonding position.

The substituted or unsubstituted divalent heterocyclic group mentioned herein is, unless otherwise specified herein, preferably a group represented by any one of formulae (TEMP-69) to (TEMP-102) below.

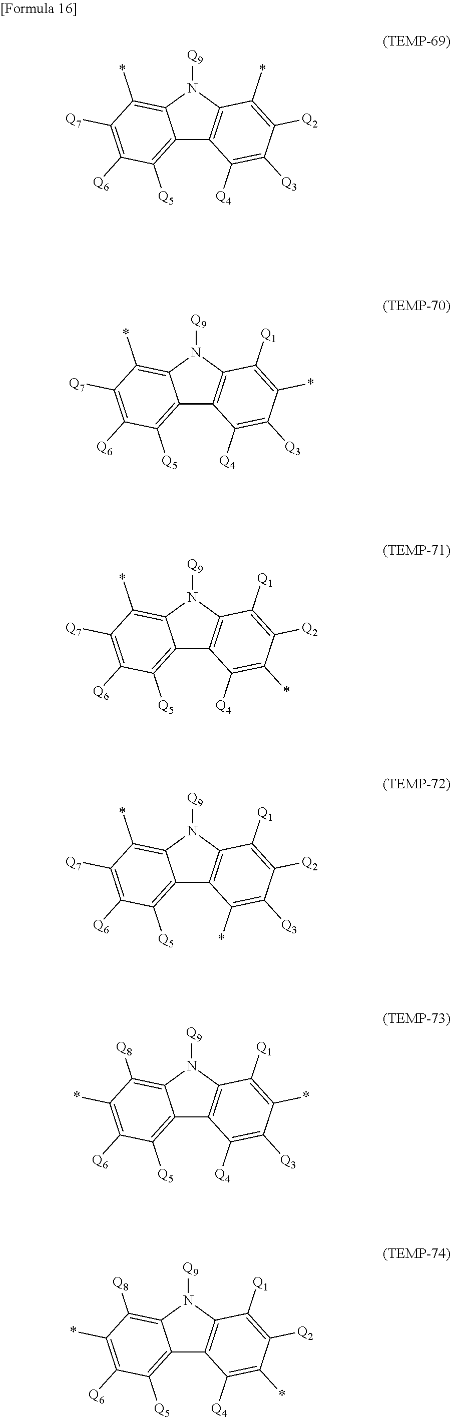

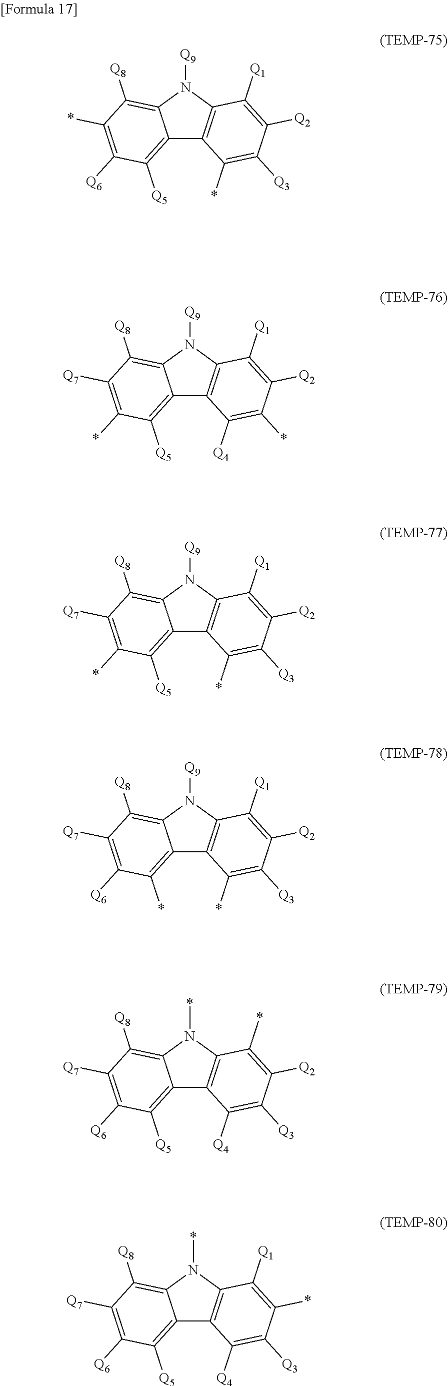

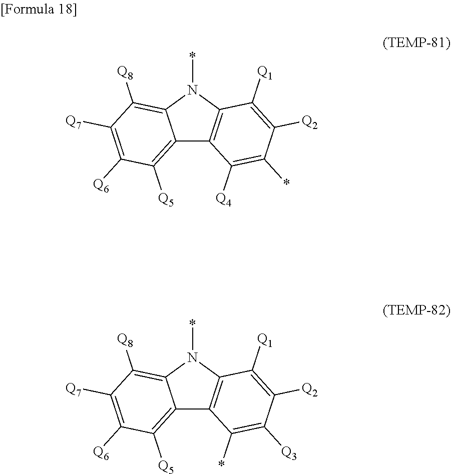

In the formulae (TEMP-69) to (TEMP-82), Q₁ to Q₉ are each independently a hydrogen atom or a substituent.

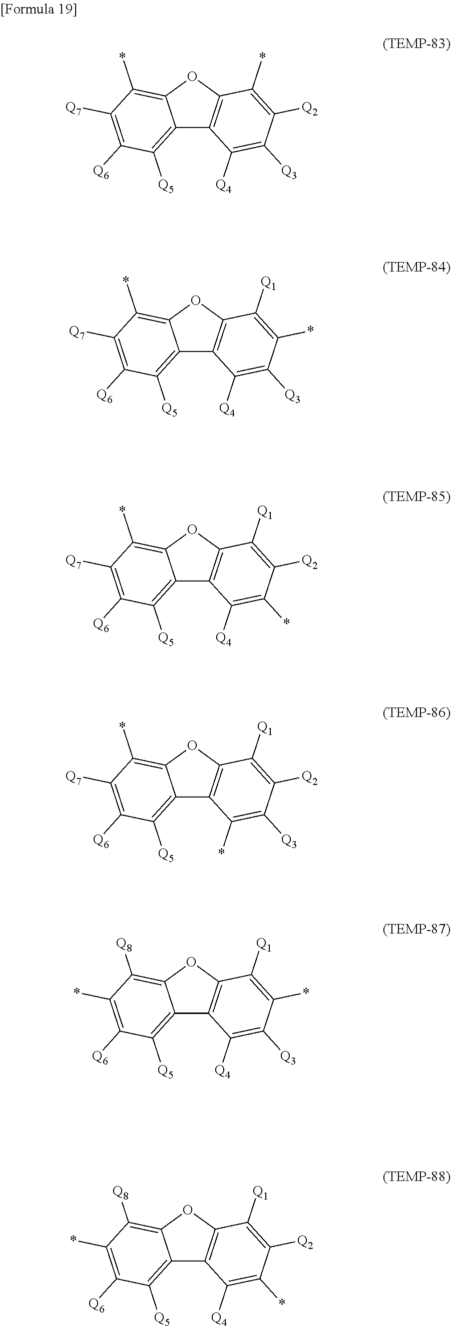

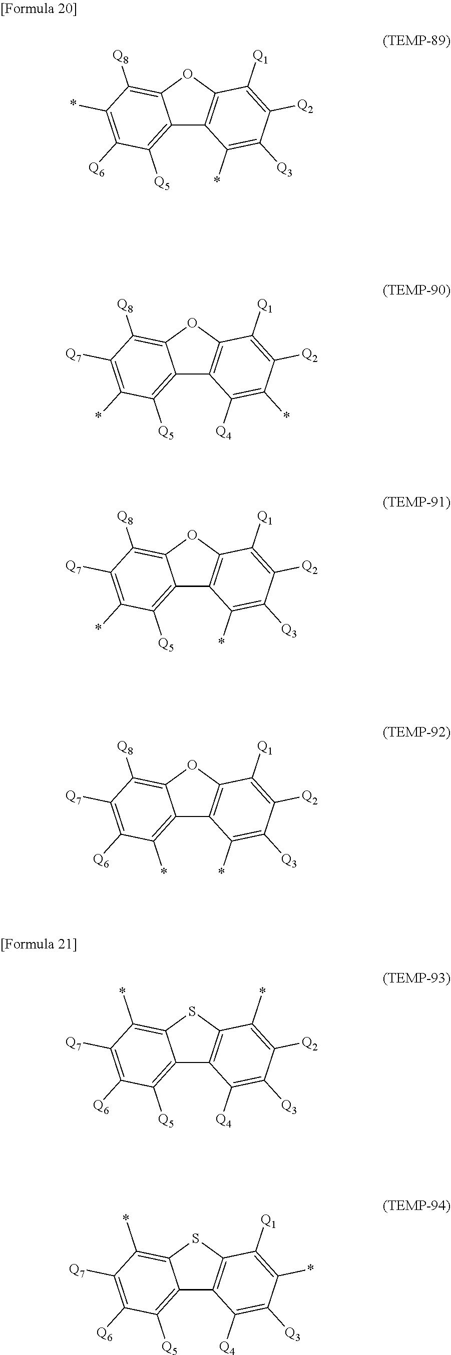

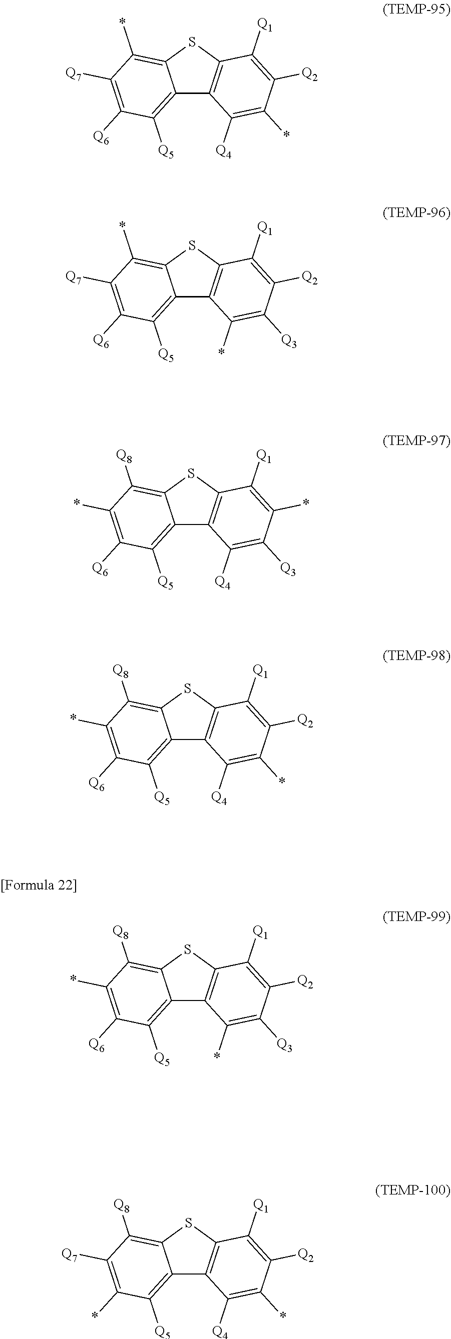

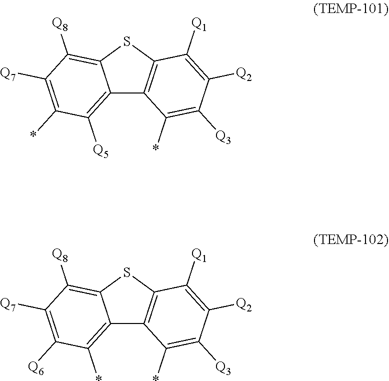

In the formulae (TEMP-83) to (TEMP-102), Q₁ to Q₈ are each independently a hydrogen atom or a substituent.

The substituent mentioned herein has been described above.

### Instance of “Bonded to Form Ring”

Instances where “at least one combination of adjacent two or more (of . . . ) are mutually bonded to form a substituted or unsubstituted monocyclic ring, mutually bonded to form a substituted or unsubstituted fused ring, or not mutually bonded” mentioned herein refer to instances where “at least one combination of adjacent two or more (of . . . ) are mutually bonded to form a substituted or unsubstituted monocyclic ring, “at least one combination of adjacent two or more (of . . . ) are mutually bonded to form a substituted or unsubstituted fused ring,” and “at least one combination of adjacent two or more (of . . . ) are not mutually bonded.”

Instances where “at least one combination of adjacent two or more (of . . . ) are mutually bonded to form a substituted or unsubstituted monocyclic ring” and “at least one combination of adjacent two or more (of . . . ) are mutually bonded to form a substituted or unsubstituted fused ring” mentioned herein (these instances will be sometimes collectively referred to as an instance of “bonded to form a ring” hereinafter) will be described below. An anthracene compound having a basic skeleton in a form of an anthracene ring and represented by a formula (TEMP-103) below will be used as an example for the description.

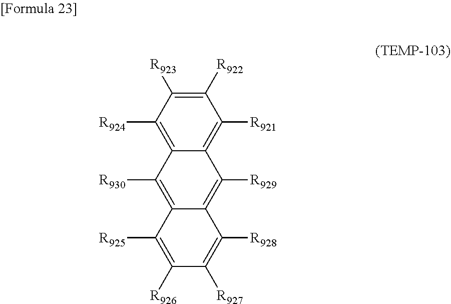

For instance, when “at least one combination of adjacent two or more of R₉₂₁ to R₉₃₀ are mutually bonded to form a ring,” the combination of adjacent ones of R₉₂₁ to R₉₃₀ (i.e. the combination at issue) is a combination of R₉₂₁ and R₉₂₂, a combination of R₉₂₂ and R₉₂₃, a combination of R₉₂₃ and R₉₂₄, a combination of R₉₂₄ and R₉₃₀, a combination of R₉₃₀ and R₉₂₅, a combination of R₉₂₅ and R₉₂₆, a combination of R₉₂₆ and R₉₂₇, a combination of R₉₂₇ and R₉₂₈, a combination of R₉₂₈ and R₉₂₉, or a combination of R₉₂₉ and R₉₂₁.

The term “at least one combination” means that two or more of the above combinations of adjacent two or more of R₉₂₁ to R₉₃₀ may simultaneously form rings. For instance, when R₉₂₁ and R₉₂₂ are mutually bonded to form a ring Q_{A} and R₉₂₅ and R₉₂₆ are simultaneously mutually bonded to form a ring Q_{B}, the anthracene compound represented by the formula (TEMP-103) is represented by a formula (TEMP-104) below.

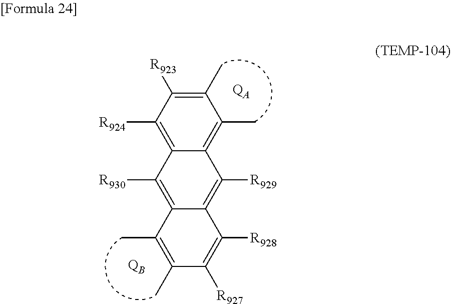

The instance where the “combination of adjacent two or more” form a ring means not only an instance where the “two” adjacent components are bonded but also an instance where adjacent “three or more” are bonded. For instance, R₉₂₁ and R₉₂₂ are mutually bonded to form a ring Q_{A} and R₉₂₂ and R₉₂₃ are mutually bonded to form a ring Q_{C}, and mutually adjacent three components (R₉₂₁, R₉₂₂ and R₉₂₃) are mutually bonded to form a ring fused to the anthracene basic skeleton. In this case, the anthracene compound represented by the formula (TEMP-103) is represented by a formula (TEMP-105) below. In the formula (TEMP-105) below, the ring Q_{A} and the ring Q_{C} share R₉₂₂.

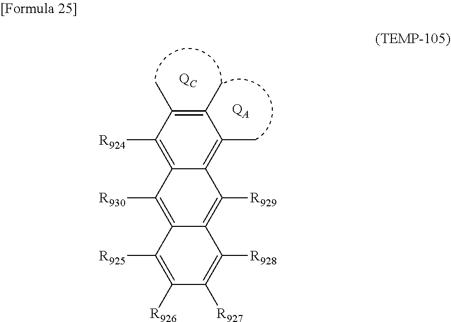

The formed “monocyclic ring” or “fused ring” may be, in terms of the formed ring in itself, a saturated ring or an unsaturated ring. When the “combination of adjacent two” form a “monocyclic ring” or a “fused ring,” the “monocyclic ring” or “fused ring” may be a saturated ring or an unsaturated ring. For instance, the ring Q_{A} and the ring Q_{B} formed in the formula (TEMP-104) are each independently a “monocyclic ring” or a “fused ring.” Further, the ring Q_{A} and the ring Q_{C} formed in the formula (TEMP-105) are each a “fused ring.” The ring Q_{A} and the ring Q_{C} in the formula (TEMP-105) are fused to form a fused ring. When the ring Q_{A} in the formula (TEMP-104) is a benzene ring, the ring Q_{A} is a monocyclic ring. When the ring Q_{A} in the formula (TEMP-104) is a naphthalene ring, the ring Q_{A} is a fused ring.

The “unsaturated ring” represents an aromatic hydrocarbon ring or an aromatic heterocycle. The “saturated ring” represents an aliphatic hydrocarbon ring or a non-aromatic heterocycle.

Specific examples of the aromatic hydrocarbon ring include a ring formed by terminating a bond of a group in the specific example of the specific example group G1 with a hydrogen atom.

Specific examples of the aromatic heterocycle include a ring formed by terminating a bond of an aromatic heterocyclic group in the specific example of the specific example group G2 with a hydrogen atom.

Specific examples of the aliphatic hydrocarbon ring include a ring formed by terminating a bond of a group in the specific example of the specific example group G6 with a hydrogen atom.

The phrase “to form a ring” herein means that a ring is formed only by a plurality of atoms of a basic skeleton, or by a combination of a plurality of atoms of the basic skeleton and one or more optional atoms. For instance, the ring Q_{A} formed by mutually bonding R₉₂₁ and R₉₂₂ shown in the formula (TEMP-104) is a ring formed by a carbon atom of the anthracene skeleton bonded to R₉₂₁, a carbon atom of the anthracene skeleton bonded to R₉₂₂, and one or more optional atoms. Specifically, when the ring Q_{A} is a monocyclic unsaturated ring formed by R₉₂₁ and R₉₂₂, the ring formed by a carbon atom of the anthracene skeleton bonded to R₉₂₁, a carbon atom of the anthracene skeleton bonded to R₉₂₂, and four carbon atoms is a benzene ring.

The “optional atom” is, unless otherwise specified herein, preferably at least one atom selected from the group consisting of a carbon atom, nitrogen atom, oxygen atom, and sulfur atom. A bond of the optional atom (e.g. a carbon atom and a nitrogen atom) not forming a ring may be terminated by a hydrogen atom or the like or may be substituted by an “optional substituent” described later. When the ring includes an optional element other than carbon atom, the resultant ring is a heterocycle.

The number of “one or more optional atoms” forming the monocyclic ring or fused ring is, unless otherwise specified herein, preferably in a range from 2 to 15, more preferably in a range from 3 to 12, further preferably in a range from 3 to 5.

Unless otherwise specified herein, the ring, which may be a “monocyclic ring” or “fused ring,” is preferably a “monocyclic ring.”

Unless otherwise specified herein, the ring, which may be a “saturated ring” or “unsaturated ring,” is preferably an “unsaturated ring.”

Unless otherwise specified herein, the “monocyclic ring” is preferably a benzene ring.

Unless otherwise specified herein, the “unsaturated ring” is preferably a benzene ring.

When “at least one combination of adjacent two or more” (of . . . ) are “mutually bonded to form a substituted or unsubstituted monocyclic ring” or “mutually bonded to form a substituted or unsubstituted fused ring,” unless otherwise specified herein, at least one combination of adjacent two or more of components are preferably mutually bonded to form a substituted or unsubstituted “unsaturated ring” formed of a plurality of atoms of the basic skeleton, and 1 to 15 atoms of at least one element selected from the group consisting of carbon, nitrogen, oxygen and sulfur.

When the “monocyclic ring” or the “fused ring” has a substituent, the substituent is the substituent described in later-described “optional substituent.” When the “monocyclic ring” or the “fused ring” has a substituent, specific examples of the substituent are the substituents described in the above under the subtitle “Substituent Mentioned Herein.”

When the “saturated ring” or the “unsaturated ring” has a substituent, the substituent is the substituent described in later-described “optional substituent.” When the “monocyclic ring” or the “fused ring” has a substituent, specific examples of the substituent are the substituents described in the above under the subtitle “Substituent Mentioned Herein.”

The above is the description for the instances where “at least one combination of adjacent two or more (of . . . ) are mutually bonded to form a substituted or unsubstituted monocyclic ring” and “at least one combination of adjacent two or more (of . . . ) are mutually bonded to form a substituted or unsubstituted fused ring” mentioned herein (sometimes referred to as an instance of “bonded to form a ring”).

### Substituent for Substituted or Unsubstituted Group

In an exemplary embodiment herein, the substituent for the substituted or unsubstituted group (hereinafter sometimes referred to as an “optional substituent”), is for instance, a group selected from the group consisting of an unsubstituted alkyl group having 1 to 50 carbon atoms, an unsubstituted alkenyl group having 2 to 50 carbon atoms, an unsubstituted alkynyl group having 2 to 50 carbon atoms, an unsubstituted cycloalkyl group having 3 to 50 ring carbon atoms, —Si(R₉₀₁)(R₉₀₂)(R₉₀₃), —O—(R₉₀₄), —S—(R₉₀₅), —N(R₉₀₆)(R₉₀₇), a halogen atom, a cyano group, a nitro group, an unsubstituted aryl group having 6 to 50 ring carbon atoms, and an unsubstituted heterocyclic group having 5 to 50 ring atoms,
R₉₀₁ to R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when two or more R₉₀₁ are present, the two or more R₉₀₁ are mutually the same or different;
when two or more R₉₀₂ are present, the two or more R₉₀₂ are mutually the same or different;
when two or more R₉₀₃ are present, the two or more R₉₀₃ are mutually the same or different;
when two or more R₉₀₄ are present, the two or more R₉₀₄ are mutually the same or different;
when two or more R₉₀₅ are present, the two or more R₉₀₅ are mutually the same or different;
when two or more R₉₀₆ are present, the two or more R₉₀₆ are mutually the same or different; and
when two or more R₉₀₇ are present, the two or more R₉₀₇ are mutually the same or different.

In an exemplary embodiment, the substituent for the substituted or unsubstituted group is a group selected from the group consisting of an alkyl group having 1 to 50 carbon atoms, an aryl group having 6 to 50 ring carbon atoms, and a heterocyclic group having 5 to 50 ring atoms.

In an exemplary embodiment, the substituent for the substituted or unsubstituted group is a group selected from the group consisting of an alkyl group having 1 to 18 carbon atoms, an aryl group having 6 to 18 ring carbon atoms, and a heterocyclic group having 5 to 18 ring atoms.

Specific examples of the above optional substituent are the same as the specific examples of the substituent described in the above under the subtitle “Substituent Mentioned Herein.”

Unless otherwise specified herein, adjacent ones of the optional substituents may form a “saturated ring” or an “unsaturated ring,” preferably a substituted or unsubstituted saturated five-membered ring, a substituted or unsubstituted saturated six-membered ring, a substituted or unsubstituted unsaturated five-membered ring, or a substituted or unsubstituted unsaturated six-membered ring, more preferably a benzene ring.

Unless otherwise specified herein, the optional substituent may further include a substituent. Examples of the substituent for the optional substituent are the same as the examples of the optional substituent.

Herein, numerical ranges represented by “AA to BB” represent a range whose lower limit is the value (AA) recited before “to” and whose upper limit is the value (BB) recited after “to.”

Herein, a numerical formula represented by “A B” means that the value A is equal to the value B, or the value A is larger than the value B.

Herein, a numerical formula represented by “A s B” means that the value A is equal to the value B, or the value A is smaller than the value B.

### First Exemplary Embodiment

### Organic Electroluminescence Device

An organic electroluminescence device of the exemplary embodiment includes: a cathode; an anode; an emitting region disposed between the cathode and the anode; and a hole transporting zone disposed between the anode and the emitting region, in which the emitting region includes one or more emitting layers, the one or more emitting layers include a first emitting layer, the first emitting layer contains a first host material, a first additional host material different from the first host material, and a first luminescent compound, the hole transporting zone includes a first anode side organic layer and a second anode side organic layer, the first anode side organic layer is in direct contact with the second anode side organic layer, the first anode side organic layer and the second anode side organic layer are disposed between the anode and the emitting region in this order from a side close to the anode, the first anode side organic layer contains a first organic material and a second organic material, the first organic material and the second organic material are mutually different, a content of the first organic material in the first anode side organic layer is less than 50 mass %, the second organic material is a monoamine compound having one substituted or unsubstituted amino group in a molecule, or a diamine compound having two substituted or unsubstituted amino groups in a molecule, the second anode side organic layer contains a second hole transporting zone material, the second hole transporting zone material is a monoamine compound having one substituted or unsubstituted amino group in a molecule, or a diamine compound having two substituted or unsubstituted amino groups in a molecule, the second hole transporting zone material and the first organic material are mutually different, the second hole transporting zone material and the second organic material are mutually the same or different, the first emitting layer is an emitting layer that emits fluorescence, and a refractive index NM₁ of constituent materials contained in the first anode side organic layer and a refractive index NM₂ of a constituent material contained in the second anode side organic layer satisfy a relationship of a numerical formula below (Numerical Formula NM).

$${NM}_{1} > {NM}_{2} \qquad ( Numerical Formula NM )$$

According to the exemplary embodiment, the organic EL device has improved device performance. In an exemplary arrangement according to the exemplary embodiment, the organic EL device has improved luminous efficiency. In an exemplary arrangement according to the exemplary embodiment, the organic EL device has a longer lifetime.

### Hole Transporting Zone

Herein, a region disposed between an anode and an emitting region and including a plurality of organic layers is referred to as a hole transporting zone.

In an exemplary arrangement of the organic EL device of the exemplary embodiment, the anode is in direct contact with the hole transporting zone, and the emitting region is in direct contact with the hole transporting zone.

### First Anode Side Organic Layer and Second Anode Side Organic Layer

The hole transporting zone includes at least a first anode side organic layer and a second anode side organic layer.

In an exemplary arrangement of the organic EL device of the exemplary embodiment, when the refractive index NM₁ of the constituent materials contained in the first anode side organic layer and the refractive index NM₂ of the constituent material contained in the second anode side organic layer satisfy the relationship of the numerical formula (Numerical Formula NM), the organic EL device has improved light-extraction efficiency.

The refractive index NM₁ of the constituent materials contained in the first anode side organic layer corresponds to a refractive index of a mixture of compounds contained in the first anode side organic layer (i.e., at least the first organic material and the second organic material). When the second anode side organic layer contains a single type of compound, the refractive index NM₂ of the constituent material contained in the second anode side organic layer corresponds to a refractive index of the single type of compound. When the second anode side organic layer contains a plurality of types of compounds, the refractive index NM₂ of the constituent material contained in the second anode side organic layer corresponds to a refractive index of a mixture containing the plurality types of compounds. Refractive indices of constituent material(s) contained in any other organic layers are also defined in the same manner as described above. The refractive index can be measured by a measurement method described in Examples below.

In an exemplary arrangement of the organic EL device of the exemplary embodiment, a difference NM₁−NM₂ between the refractive index NM₁ of the constituent materials contained in the first anode side organic layer and the refractive index NM₂ of the constituent material contained in the second anode side organic layer satisfies a relationship of a numerical formula (Numerical Formula NM₁) below.

$${NM}_{1} - {NM}_{2} \geq 0.01 \qquad ( Numerical Formula NM1 )$$

In an exemplary arrangement of the organic EL device of the exemplary embodiment, the difference NM₁−NM₂ between the refractive index NM₁ of the constituent materials contained in the first anode side organic layer and the refractive index NM₂ of the constituent material contained in the second anode side organic layer satisfies a relationship of a numerical formula (Numerical Formula NM2), a numerical formula (Numerical Formula NM3), or a numerical formula (Numerical Formula NM4) below.

$${NM}_{1} - {NM}_{2} \geq 0.05 \qquad ( Numerical Formula NM2 )$$

$${NM}_{1} - {NM}_{2} \geq 0.75 \qquad ( Numerical Formula NM3 )$$

$${NM}_{1} - {NM}_{2} \geq 0.1 \qquad ( Numerical Formula NM4 )$$

In an exemplary arrangement of the organic EL device of the exemplary embodiment, the refractive index NM₁ of the constituent materials contained in the first anode side organic layer is 1.90 or more.

In an exemplary arrangement of the organic EL device of the exemplary embodiment, the refractive index of the second organic material contained in the first anode side organic layer is 1.90 or more.

In an exemplary arrangement of the organic EL device of the exemplary embodiment, the refractive index NM₁ of the constituent materials contained in the first anode side organic layer is 1.94 or more.

In an exemplary arrangement of the organic EL device of the exemplary embodiment, the refractive index of the second organic material contained in the first anode side organic layer is 1.94 or more.

In an exemplary arrangement of the organic EL device of the exemplary embodiment, the refractive index NM₂ of the constituent material contained in the second anode side organic layer is 1.89 or less.

In an exemplary arrangement of the organic EL device of the exemplary embodiment, the refractive index of the second hole transporting zone material contained in the second anode side organic layer is 1.89 or less.

In an exemplary arrangement of the organic EL device of the exemplary embodiment, the hole transporting zone consists of the first anode side organic layer and the second anode side organic layer. In this case, the total film thickness of the hole transporting zone corresponds to a total of a film thickness of the first anode side organic layer and a film thickness of the second anode side organic layer.

In an exemplary arrangement of the organic EL device of the exemplary embodiment, the first anode side organic layer has a film thickness of 20 nm or less.

In an exemplary arrangement of the organic EL device of the exemplary embodiment, the second anode side organic layer has a film thickness in a range from 30 nm to 150 nm.

In an exemplary arrangement of the organic EL device of the exemplary embodiment, the second anode side organic layer has a film thickness in a range from 30 nm to 80 nm.

In an exemplary arrangement of the organic EL device of the exemplary embodiment, the second anode side organic layer has a film thickness in a range from 80 nm to 150 nm.

In an exemplary arrangement of the organic EL device of the exemplary embodiment, the ratio of a film thickness of the second anode side organic layer to a film thickness of the first anode side organic layer preferably satisfies a relationship of a numerical formula (Numerical Formula A1) below, more preferably a numerical formula (Numerical Formula A2) below.

$$2.0 < T {L}_{2} / {TL}_{1} < 30 \qquad ( Numerical Formula A1 )$$

$$8. < T {L}_{2} / {TL}_{1} < 16 \qquad ( Numerical Formula A2 )$$

where TL₁ is a film thickness of the first anode side organic layer, TL₂ is a film thickness of the second anode side organic layer, and a unit of the film thickness is denoted by nm.

In an exemplary arrangement of the organic EL device of the exemplary embodiment, the first anode side organic layer is in direct contact with the anode.

In an exemplary arrangement of the organic EL device of the exemplary embodiment, when the hole transporting zone consists of the first anode side organic layer and the second anode side organic layer, the second anode side organic layer is in direct contact with the emitting region.

In an exemplary arrangement of the organic EL device of the exemplary embodiment, the first anode side organic layer contains no compound contained in the second anode side organic layer. An arrangement satisfying the above condition is, for instance, as follows: when a compound CA, a compound CB and a compound AA are different compounds, the first anode side organic layer contains two types of compounds (the compound CA as the first organic material and the compound CB as the second organic material) and the second anode side organic layer contains a single type of compound (the compound AA). The above condition is satisfied because both the compounds CA and CB contained in the first anode side organic layer are different from the compound AA.

On the other hand, for instance, the above condition is not satisfied when the first anode side organic layer contains two types of compounds (compound CA and compound CB) and the second anode side organic layer contains a single type of compound (compound CB), because the first anode side organic layer and the second anode side organic layer contain the same compound (compound CB).

In an exemplary arrangement of the organic EL device of the exemplary embodiment, all the compound(s) contained in the first anode side organic layer is/are different from the compound(s) contained in the second anode side organic layer.

An arrangement satisfying the above condition is, for instance, as follows: when a compound CA, a compound CB, a compound AA and a compound AB are different compounds, the second anode side organic layer contains a single type of compound (the compound AA) and the first anode side organic layer contains two types of compounds (the compound CA and the compound CB). Further, the above condition is also satisfied when the second anode side organic layer contains two types of compounds (the compound AA and the compound AB) and the first anode side organic layer contains two types of compounds (the compound CA and the compound CB). On the other hand, the above condition is not satisfied, for instance, when the second anode side organic layer contains a single type of compound (the compound AA) and the first anode side organic layer contains two types of compounds (the compound CA and the compound AA), because the first anode side organic layer and the second anode side organic layer contain the same compound (the compound AA).

In the organic EL device according to the exemplary embodiment, the second organic material contained in the first anode side organic layer is a monoamine compound having one substituted or unsubstituted amino group in a molecule, or a diamine compound having two substituted or unsubstituted amino groups in a molecule.

In the monoamine compound and the diamine compound, a nitrogen atom of an amino group is not a ring atom. When a nitrogen atom is a ring atom in a carbazole ring, an azine ring, and the like, the nitrogen atom is not a nitrogen atom as an amino group.

For instance, a compound HT-X below has two nitrogen atoms in a molecule: one nitrogen atom in the compound HT-X is a ring atom of a carbazole ring and the other nitrogen atom is not a ring atom but a nitrogen atom as an amino group. The compound HT-X is a compound having a structure in which a 9-phenyl-3-carbazolyl group is bonded to a nitrogen atom of an amino group via a linking group, that is, a monoamine compound.

A compound HT-Y below is also a compound having a structure in which a 9-carbazolyl group is bonded to a nitrogen atom of an amino group via a linking group, that is, a monoamine compound.

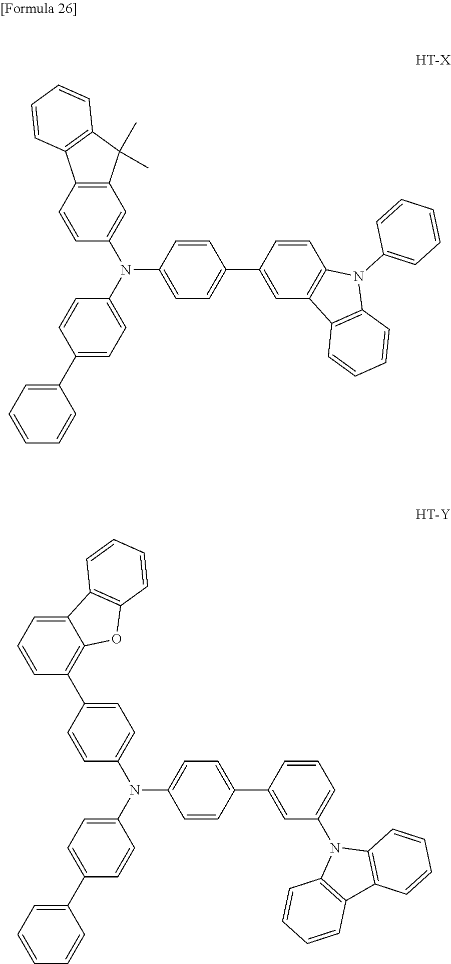 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(N(c3ccc(-c4cccc(-n5c6ccccc6c6ccccc65)c4)cc3)c3ccc(-c4cccc5c4oc4ccccc45)cc3)cc2)cc1]

In an exemplary arrangement of the organic EL device of the exemplary embodiment, the second organic material contained in the first anode side organic layer is at least one compound selected from the group consisting of a compound represented by a formula (cHT2-1), a compound represented by a formula (cHT2-2), and a compound represented by a formula (cHT2-3) below.

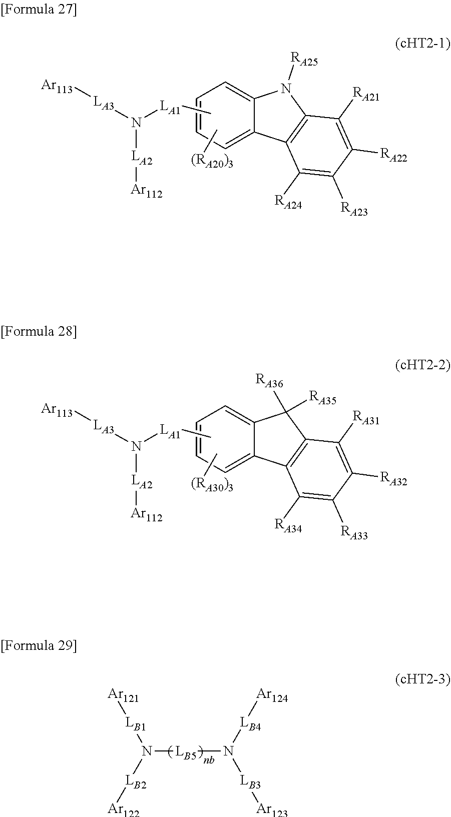

In the formulae (cHT2-1), (cHT2-2), and (cHT2-3):
Ar₁₁₂, Ar₁₁₃, Ar₁₂₁, Ar₁₂₂, Ar₁₂₃, and Ar₁₂₄ are each independently a substituted or unsubstituted aryl group having 6 to 50 ring carbon atoms, a substituted or unsubstituted heterocyclic group having 5 to 50 ring atoms, or —Si(R_{C1})(R_{C2})(R_{C3});
R_{C1}, R_{C2}, and R_{C3} are each independently a substituted or unsubstituted aryl group having 6 to 50 ring carbon atoms;
when a plurality of R_{C1} are present, the plurality of R_{C1} are mutually the same or different;
when a plurality of R_{C2} are present, the plurality of R_{C2} are mutually the same or different;
when a plurality of R_{C3} are present, the plurality of R_{C3} are mutually the same or different;
L_{A1}, L_{A2}, L_{A3}, L_{B1}, L_{B2}, L_{B3}, and L_{B4} are each independently a single bond, a substituted or unsubstituted arylene group having 6 to 50 ring carbon atoms, or a substituted or unsubstituted divalent heterocyclic group having 5 to 50 ring atoms;
nb is 1, 2, 3 or 4;
when nb is 1, L_{B5} is a substituted or unsubstituted arylene group having 6 to 50 ring carbon atoms, or a substituted or unsubstituted divalent heterocyclic group having 5 to 50 ring atoms;
when nb is 2, 3, or 4, a plurality of L_{B5} are mutually the same or different;
when nb is 2, 3, or 4, a plurality of L_{B5} are mutually bonded to form a substituted or unsubstituted monocyclic ring, mutually bonded to form a substituted or unsubstituted fused ring, or not mutually bonded;
L_{B5} forming neither the substituted or unsubstituted monocyclic ring nor the substituted or unsubstituted fused ring is a substituted or unsubstituted arylene group having 6 to 50 ring carbon atoms, or a substituted or unsubstituted divalent heterocyclic group having 5 to 50 ring atoms;
a combination of R_{A35} and R_{A36} are mutually bonded to form a substituted or unsubstituted monocyclic ring, mutually bonded to form a substituted or unsubstituted fused ring, or not mutually bonded;
R_{A25}, and R_{A35} and R_{A36} forming neither the substituted or unsubstituted monocyclic ring nor the substituted or unsubstituted fused ring are each independently a hydrogen atom, a cyano group, a substituted or unsubstituted alkyl group having 1 to 50 carbon atoms, a substituted or unsubstituted alkyl halide group having 1 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a substituted or unsubstituted aryl group having 6 to 50 ring carbon atoms, or a substituted or unsubstituted heterocyclic group having 5 to 50 ring atoms;
at least one combination of adjacent two or more of R_{A20} to R_{A24} are mutually bonded to form a substituted or unsubstituted monocyclic ring, mutually bonded to form a substituted or unsubstituted fused ring, or not mutually bonded;
at least one combination of adjacent two or more of R_{A30} to R_{A34} are mutually bonded to form a substituted or unsubstituted monocyclic ring, mutually bonded to form a substituted or unsubstituted fused ring, or not mutually bonded; and
R_{A20} to R_{A24} and R_{A30} to R_{A34} forming neither the substituted or unsubstituted monocyclic ring nor the substituted or unsubstituted fused ring are each independently a hydrogen atom, a cyano group, a substituted or unsubstituted alkyl group having 1 to 50 carbon atoms, a substituted or unsubstituted alkyl halide group having 1 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a substituted or unsubstituted aryl group having 6 to 50 ring carbon atoms, or a substituted or unsubstituted heterocyclic group having 5 to 50 ring atoms;
in the compounds represented by the formulae (cHT2-1), (cHT2-2), and (cHT2-3), R₉₀₁ to R₉₀₄ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different; and
when a plurality of R₉₀₄ are present, the plurality of R₉₀₄ are mutually the same or different.

In the compounds represented by the formulae (cHT2-1), (cHT2-2), and (cHT2-3) in an exemplary arrangement of the organic EL device of the exemplary embodiment, the substituent for the “substituted or unsubstituted” group is not a group represented by —N(R_{C6})(R_{C7}), and R_{C6} and R_{C7} are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

In an exemplary arrangement of the organic EL device of the exemplary embodiment, the second organic material is a monoamine compound having one substituted or unsubstituted amino group in a molecule.

In an exemplary arrangement of the organic EL device of the exemplary embodiment, the compound represented by the formula (cHT2-1) and the compound represented by the formula (cHT2-2) are each a monoamine compound.

In an exemplary arrangement of the organic EL device of the exemplary embodiment, the second organic material as the compound contained in the first anode side organic layer is a diamine compound having two substituted or unsubstituted amino groups in a molecule.

In an exemplary arrangement of the organic EL device of the exemplary embodiment, the compound represented by the formula (cHT2-3) is a diamine compound.

In an exemplary arrangement of the organic EL device of the exemplary embodiment, the second organic material is at least one compound selected from the group consisting of a compound represented by the formula (cHT2-1), a compound represented by the formula (cHT2-2), and a compound represented by the formula (cHT2-3).

In an exemplary arrangement of the organic EL device of the exemplary embodiment, the second organic material has at least one group selected from the group consisting of a group represented by a formula (2-a), a group represented by a formula (2-b), a group represented by a formula (2-c), a group represented by a formula (2-d), a group represented by a formula (2-e), and a group represented by a formula (2-f) below.

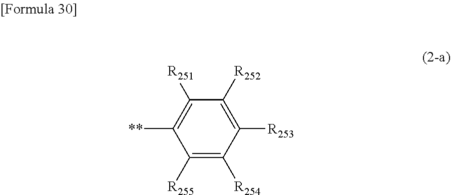

In the formula (2-a):
none of a combination(s) of adjacent two or more of R₂₅₁ to R₂₅₅ are bonded to each other;
R₂₅₁ to R₂₅₅ are each independently a hydrogen atom, or a substituted or unsubstituted alkyl group having 1 to 6 carbon atoms; and
** represents a bonding position.

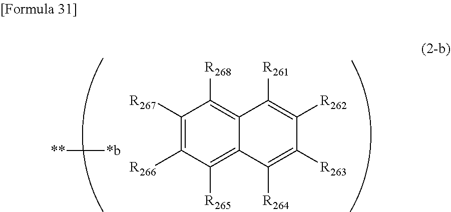

In the formula (2-b):
one of R₂₆₁ to R₂₆₈ is a single bond with *b;
none of a combination(s) of adjacent two or more of R₂₆₁ to R₂₆₈ not being the single bond with *b are bonded to each other;
R₂₆₁ to R₂₆₈ not being the single bond with *b are each independently a hydrogen atom, a substituted or unsubstituted alkyl group having 1 to 6 carbon atoms, or a substituted or unsubstituted aryl group having 6 to 12 ring carbon atoms; and
** represents a bonding position.

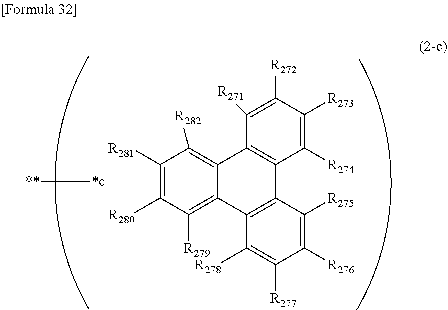

In the formula (2-c):
one of R₂₇₁ to R₂₈₂ is a single bond with *c;
none of a combination(s) of adjacent two or more of R₂₇₁ to R₂₈₂ not being the single bond with *c are bonded to each other;
R₂₇₁ to R₂₈₂ not being the single bond with *c are each independently a hydrogen atom, a substituted or unsubstituted alkyl group having 1 to 6 carbon atoms, or a substituted or unsubstituted aryl group having 6 to 12 ring carbon atoms; and
** represents a bonding position.

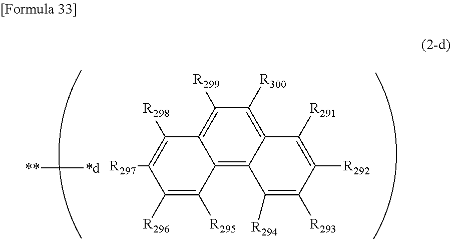

In the formula (2-d):
one of R₂₉₁ to R₃₀₀ is a single bond with *d;
none of a combination(s) of adjacent two or more of R₂₉₁ to R₃₀₀ not being the single bond with *d are bonded to each other;
R₂₉₁ to R₃₀₀ not being the single bond with *d are each independently a hydrogen atom, a substituted or unsubstituted alkyl group having 1 to 6 carbon atoms, or a substituted or unsubstituted aryl group having 6 to 12 ring carbon atoms; and
** represents a bonding position.

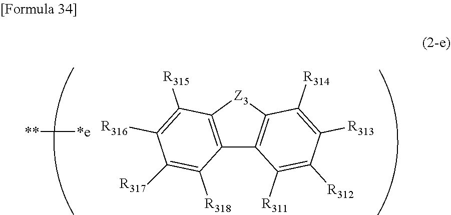

In the formula (2-e):
Z₃ is an oxygen atom, a sulfur atom, NR₃₁₉, or C(R₃₂₀)(R₃₂₁);
one of R₃₁₁ to R₃₂₁ is a single bond with *e, or one of carbon atoms of a substituted or unsubstituted benzene ring below formed by mutually bonding a combination of adjacent two or more of R₃₁₁ to R₃₁₈ is bonded to *e by a single bond;
a combination of adjacent two or more of R₃₁₁ to R₃₁₈ not being the single bond with *e are mutually bonded to form a substituted or unsubstituted benzene ring, or not mutually bonded;
R₃₁₁ to R₃₁₈ not being the single bond with *e and not forming the substituted or unsubstituted benzene ring are each independently a hydrogen atom, a substituted or unsubstituted alkyl group having 1 to 6 carbon atoms, a substituted or unsubstituted aryl group having 6 to 12 ring carbon atoms, or a substituted or unsubstituted heterocyclic group having 5 to 10 ring atoms;
R₃₁₉ not being the single bond with *e is a hydrogen atom, a substituted or unsubstituted alkyl group having 1 to 6 carbon atoms, or a substituted or unsubstituted aryl group having 6 to 12 ring carbon atoms;
a combination of R₃₂₀ and R₃₂₁ not being the single bond with *e are mutually bonded to form a substituted or unsubstituted monocyclic ring, mutually bonded to form a substituted or unsubstituted fused ring, or not mutually bonded;
R₃₂₀ and R₃₂₁ not being the single bond with *e, not forming the substituted or unsubstituted monocyclic ring, and not forming the substituted or unsubstituted fused ring are each independently a hydrogen atom, a substituted or unsubstituted alkyl group having 1 to 6 carbon atoms, or a substituted or unsubstituted aryl group having 6 to 12 ring carbon atoms; and
** represents a bonding position.

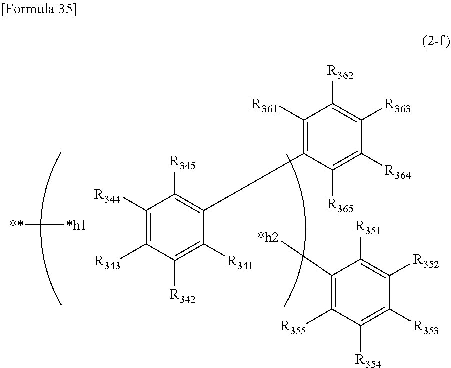

In the formula (2-f):
one of R₃₄₁ to R₃₄₅ is a single bond with *h1 and another one of R₃₄₁ to R₃₄₅ is a single bond with *h2;
none of a combination(s) of adjacent two or more of R₃₄₁ to R₃₄₅ not being the single bond with *h1 and not being the single bond with *h2 are bonded to each other;
at least one combination of adjacent two or more of R₃₅₁ to R₃₅₅ are mutually bonded to form a substituted or unsubstituted monocyclic ring, mutually bonded to form a substituted or unsubstituted fused ring, or not mutually bonded;
at least one combination of adjacent two or more of R₃₆₁ to R₃₆₅ are mutually bonded to form a substituted or unsubstituted monocyclic ring, mutually bonded to form a substituted or unsubstituted fused ring, or not mutually bonded;
R₃₄₁ to R₃₄₅ not being the single bond with *h1 and not being the single bond with *h2 as well as R₃₅₁ to R₃₅₅ and R₃₆₁ to R₃₆₅ forming neither the substituted or unsubstituted monocyclic ring nor the substituted or unsubstituted fused ring are each independently a hydrogen atom, a substituted or unsubstituted alkyl group having 1 to 6 carbon atoms, or a substituted or unsubstituted aryl group having 6 to 12 ring carbon atoms; and
** represents a bonding position.

In an exemplary arrangement of the organic EL device of the exemplary embodiment, the second organic material is a monoamine compound having one substituted or unsubstituted amino group in a molecule, and the group represented by the formula (2-a), the group represented by the formula (2-b), the group represented by the formula (2-c), the group represented by the formula (2-d), the group represented by the formula (2-e), and the group represented by the formula (2-f) are each independently bonded directly, through a phenylene group, or through a biphenylene group to a nitrogen atom of an amino group of the monoamine compound.

In an exemplary arrangement of the organic EL device of the exemplary embodiment, the group represented by the formula (2-e) is a group represented by a formula (2-e1), a formula (2-e2), or a formula (2-e3) below.

In an exemplary arrangement of the organic EL device of the exemplary embodiment, the second organic material is a compound represented by the formula (cHT2-1) in which at least one of Ar₁₁₂ or Ar₁₁₃ is a group selected from the group consisting of a group represented by the formula (2-a), a group represented by the formula (2-b), a group represented by the formula (2-c), a group represented by the formula (2-d), a group represented by the formula (2-e), and a group represented by the formula (2-f).

In an exemplary arrangement of the organic EL device of the exemplary embodiment, the second organic material is a compound represented by the formula (cHT2-2) in which at least one of Ar₁₁₂ or Ar₁₁₃ is a group selected from the group consisting of a group represented by the formula (2-a), a group represented by the formula (2-b), a group represented by the formula (2-c), a group represented by the formula (2-d), a group represented by the formula (2-e), and a group represented by the formula (2-f).

In an exemplary arrangement of the organic EL device of the exemplary embodiment, the second organic material is a compound represented by the formula (cHT2-3) in which at least one of Ar₁₂₁, Ar₁₂₂, Ar₁₂₃, or Ar₁₂₄ is a group selected from the group consisting of a group represented by the formula (2-a), a group represented by the formula (2-b), a group represented by the formula (2-c), a group represented by the formula (2-d), a group represented by the formula (2-e), and a group represented by the formula (2-f).

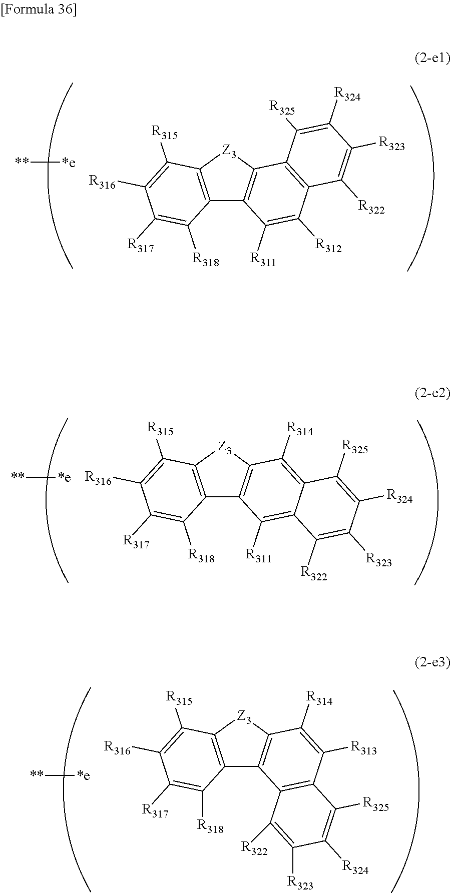

In the formulae (2-e1), (2-e2), and (2-e3):
Z₃ in an oxygen atom, a sulfur atom, NR₃₁₉, or C(R₃₂₀)(R₃₂₁);
one of R₃₁₁ to R₃₂₅ is a single bond with *e;
R₃₁₁ to R₃₁₈ and R₃₂₂ and R₃₂₅ not being the single bond with *e are each independently a hydrogen atom, a substituted or unsubstituted alkyl group having 1 to 6 carbon atoms, a substituted or unsubstituted aryl group having 6 to 12 ring carbon atoms, or a substituted or unsubstituted heterocyclic group having 5 to 10 ring atoms;
R₃₁₉ not being the single bond with *e is a hydrogen atom, a substituted or unsubstituted alkyl group having 1 to 6 carbon atoms, or a substituted or unsubstituted aryl group having 6 to 12 ring carbon atoms;
a combination of R₃₂₀ and R₃₂₁ not being the single bond with *e are mutually bonded to form a substituted or unsubstituted monocyclic ring, mutually bonded to form a substituted or unsubstituted fused ring, or not mutually bonded;
R₃₂₀ and R₃₂₁ not being the single bond with *e, not forming the substituted or unsubstituted monocyclic ring, and not forming the substituted or unsubstituted fused ring are each independently a hydrogen atom, a substituted or unsubstituted alkyl group having 1 to 6 carbon atoms, or a substituted or unsubstituted aryl group having 6 to 12 ring carbon atoms; and
** each represent a bonding position.

In an exemplary arrangement of the organic EL device of the exemplary embodiment, ** in the formulae (2-a), (2-b), (2-c), (2-d), (2-e), (2-f), (2-e1), (2-e2), and (2-e3) are each independently a bonding position to L_{A2}, L_{A3}, L_{B1}, L_{B2}, L_{B3}, or L_{B4} or a bonding position to a nitrogen atom of an amino group.

In an exemplary arrangement of the organic EL device of the exemplary embodiment, the compound contained in the first anode side organic layer (second organic material) is a compound having no thiophene ring in a molecule.

In an exemplary arrangement of the organic EL device of the exemplary embodiment, the second organic material as the compound contained in the first anode side organic layer is occasionally referred to as a first hole transporting zone material.

In an exemplary arrangement of the organic EL device of the exemplary embodiment, the content of the first organic material in the first anode side organic layer is less than 50 mass %.

In an exemplary arrangement of the organic EL device of the exemplary embodiment, the content of the first organic material in the first anode side organic layer is 15 mass % or less.

In an exemplary arrangement of the organic EL device of the exemplary embodiment, the content of the first organic material in the first anode side organic layer is 5 mass % or more.

In an exemplary arrangement of the organic EL device of the exemplary embodiment, the content of the first organic material in the first anode side organic layer is 30 mass % or less, 20 mass % or less, or 15 mass % or less.

In an exemplary arrangement of the organic EL device of the exemplary embodiment, the content of the second organic material in the first anode side organic layer is more than 50 mass %, 70 mass % or more, 80 mass % or more, or 85 mass % or more.

In an exemplary arrangement of the organic EL device of the exemplary embodiment, the content of the second organic material in the first anode side organic layer is 95 mass % or less.

The total of the content of the first organic material and the content of the second organic material in the first anode side organic layer is 100 mass % or less.

In an exemplary arrangement of the organic EL device of the exemplary embodiment, the first anode side organic layer contains a doped compound as the first organic material and a first hole transporting zone material as the second organic material.

In an exemplary arrangement of the organic EL device of the exemplary embodiment, the doped compound has at least one of a first cyclic structure represented by a formula (P11) below or a second cyclic structure represented by a formula (P12) below.

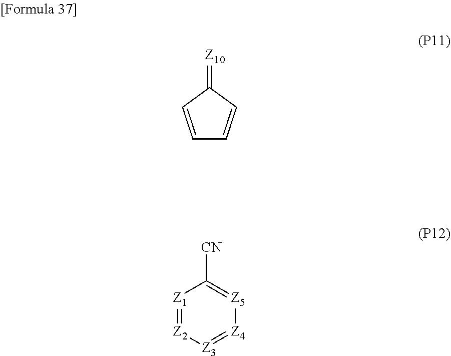

The first cyclic structure represented by the formula (P11) is fused, in a molecule of the doped compound, to at least one cyclic structure of a substituted or unsubstituted aromatic hydrocarbon ring having 6 to 50 ring carbon atoms or a substituted or unsubstituted heterocycle having 5 to 50 ring atoms, and
a structure represented by ═Z₁₀ is represented by a formula (11a), (11b), (11c), (11d), (11e), (11f), (11g), (11h), (11i), (11j), (11k) or (11m) below.

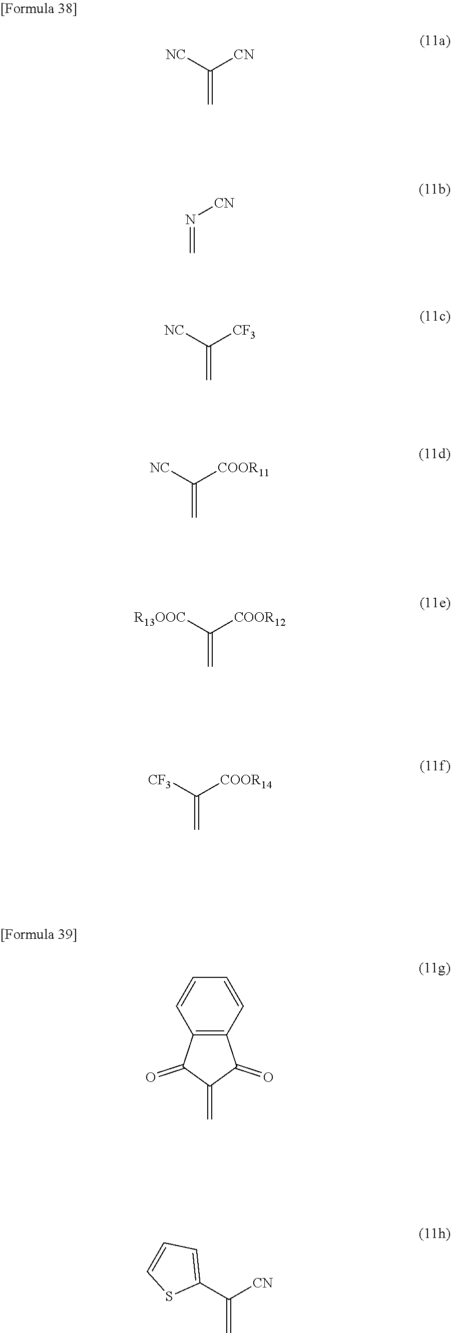

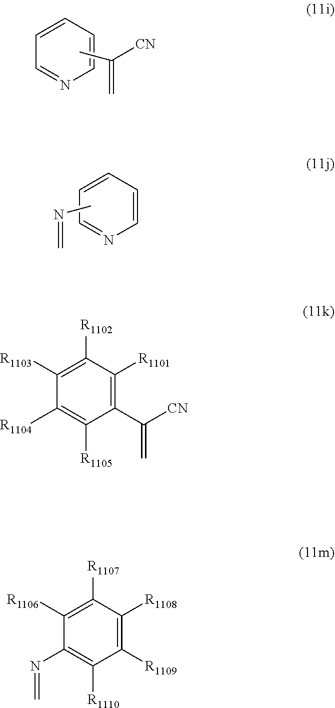

In the formula (11a), (11b), (11c), (11d), (11e), (11f), (11g), (11h), (11i), (11j), (11k) or (11m), R₁₁ to R₁₄ and R₁₁₀₁ to R₁₁₁₀ are each independently a hydrogen atom, a halogen atom, a hydroxy group, a cyano group, a substituted or unsubstituted alkyl group having 1 to 50 carbon atoms, a substituted or unsubstituted alkyl halide group having 1 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aryl group having 6 to 50 ring carbon atoms, or a substituted or unsubstituted heterocyclic group having 5 to 50 ring atoms. In the formula (P12), Z₁ to Z₅ are each independently a nitrogen atom, a carbon atom bonded to R₁₅, or a carbon atom bonded to another atom in the molecule of the doped compound;
at least one of Z₁ to Z₅ is a carbon atom bonded to another atom in the molecule of the doped compound;
R₁₅ is selected from the group consisting of a hydrogen atom, a halogen atom, a cyano group, a substituted or unsubstituted alkyl group having 1 to 50 carbon atoms, a substituted or unsubstituted alkyl halide group having 1 to 50 carbon atoms, a substituted or unsubstituted cycloalkyl group having 3 to 50 ring carbon atoms, a substituted or unsubstituted aryl group having 6 to 50 ring carbon atoms, a substituted or unsubstituted heterocyclic group having 5 to 50 ring atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted alkenyl group having 2 to 50 carbon atoms, a substituted or unsubstituted aralkyl group having 7 to 50 carbon atoms, a carboxy group, a substituted or unsubstituted ester group, a substituted or unsubstituted carbamoyl group, a nitro group, and a substituted or unsubstituted siloxanyl group; and
when a plurality of R₁₅ are present, the plurality of R₁₅ are mutually the same or different.

In the doped compound, R₉₀₁ to R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different; and
when a plurality of R₉₀₇ are present, the plurality of R₉₀₇ are mutually the same or different.

An ester group herein is at least one group selected from the group consisting of an alkyl ester group and an aryl ester group.

An alkyl ester group herein is represented, for instance, by —C(═O)OR^{E}. R^{E} is exemplified by a substituted or unsubstituted alkyl group having 1 to 50 carbon atoms (preferably 1 to 10 carbon atoms).

An aryl ester group herein is represented, for instance, by —C(═O)OR^{Ar}. R^{Ar} is exemplified by a substituted or unsubstituted aryl group having 6 to 30 ring carbon atoms.

A siloxanyl group herein, which is a silicon compound group through an ether bond, is exemplified by a trimethylsiloxanyl group.

A carbamoyl group herein is represented by —CONH₂.

A substituted carbamoyl group herein is represented, for instance, by —CONH—Ar^{C} or —CONH—R^{C}. Ar^{C} is, for instance, at least one group selected from the group consisting of a substituted or unsubstituted aryl group having 6 to 50 ring carbon atoms (preferably 6 to 10 ring carbon atoms) and a heterocyclic group having 5 to 50 ring atoms (preferably 5 to 14 ring atoms). Ar^{C} may be a group in which a substituted or unsubstituted aryl group having 6 to 50 ring carbon atoms is bonded to a substituted or unsubstituted heterocyclic group having 5 to 50 ring atoms.

R^{C} is exemplified by a substituted or unsubstituted alkyl group having 1 to 50 carbon atoms (preferably 1 to 6 carbon atoms).

In the doped compound, the groups specified to be “substituted or unsubstituted” are each preferably an “unsubstituted” group.

In an exemplary arrangement of the organic EL device of the exemplary embodiment, the second organic material is a monoamine compound having only one substituted or unsubstituted amino group in a molecule.

In an exemplary arrangement of the organic EL device of the exemplary embodiment, the second organic material contains at least one compound selected from the group consisting of a compound represented by the formula (cHT2-1) and a compound represented by the formula (cHT2-2).

In an exemplary arrangement of the organic EL device of the exemplary embodiment, the compound represented by the formula (cHT2-1) and the compound represented by the formula (cHT2-2), as the second organic material, are each a monoamine compound.

In the organic EL device according to the exemplary embodiment, the second hole transporting zone material is a monoamine compound having one substituted or unsubstituted amino group in a molecule, or a diamine compound having two substituted or unsubstituted amino groups in a molecule.

In an exemplary arrangement of the organic EL device of the exemplary embodiment, the second hole transporting zone material is a monoamine compound having one substituted or unsubstituted amino group in a molecule.

In an exemplary arrangement of the organic EL device of the exemplary embodiment, the second hole transporting zone material is at least one compound selected from the group consisting of a compound represented by a formula (C1) and a compound represented by a formula (C3) below.

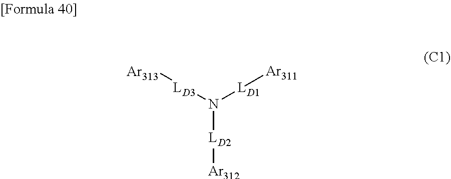

In the formula (C1):
Ar₃₁₁, Ar₃₁₂, and Ar₃₁₃ are each independently a substituted or unsubstituted aryl group having 6 to 50 ring carbon atoms, a substituted or unsubstituted heterocyclic group having 5 to 50 ring atoms, or —Si(R_{C1})(R_{C2})(R_{C3});
R_{C1}, R_{C2}, and R_{C3} are each independently a substituted or unsubstituted aryl group having 6 to 50 ring carbon atoms;
when a plurality of R_{C1} are present, the plurality of R_{C1} are mutually the same or different;
when a plurality of R_{C2} are present, the plurality of R_{C2} are mutually the same or different;
when a plurality of R_{C3} are present, the plurality of R_{C3} are mutually the same or different; and
L_{D1}, L_{D2}, and L_{D3} are each independently a single bond, a substituted or unsubstituted arylene group having 6 to 50 ring carbon atoms, or a substituted or unsubstituted divalent heterocyclic group having 5 to 50 ring atoms.

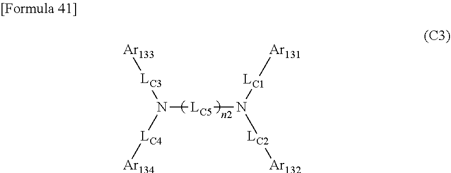

In the formula (C3):
L_{C1}, L_{C2}, L_{C3}, and L_{C4} are each independently a single bond, a substituted or unsubstituted arylene group having 6 to 50 ring carbon atoms, or a substituted or unsubstituted divalent heterocyclic group having 5 to 50 ring atoms;
n2 is 1, 2, 3, or 4;
when n2 is 1, L_{C5} is a substituted or unsubstituted arylene group having 6 to 50 ring carbon atoms, or a substituted or unsubstituted divalent heterocyclic group having 5 to 50 ring atoms;
when n2 is 2, 3, or 4, a plurality of L_{C5} are mutually the same or different;
when n2 is 2, 3, or 4, a plurality of L_{C5} are mutually bonded to form a substituted or unsubstituted monocyclic ring, mutually bonded to form a substituted or unsubstituted fused ring, or not mutually bonded;
L_{C5} forming neither the substituted or unsubstituted monocyclic ring nor the substituted or unsubstituted fused ring is a substituted or unsubstituted arylene group having 6 to 50 ring carbon atoms, or a substituted or unsubstituted divalent heterocyclic group having 5 to 50 ring atoms;
Ar₁₃₁, Ar₁₃₂, Ar₁₃₃, and Ar₁₃₄ are each independently a substituted or unsubstituted aryl group having 6 to 50 ring carbon atoms, a substituted or unsubstituted heterocyclic group having 5 to 50 ring atoms, or —Si(R_{C1})(R_{C2})(R_{C3});
R_{C1}, R_{C2}, and R_{C3} are each independently a substituted or unsubstituted aryl group having 6 to 50 ring carbon atoms;
when a plurality of R_{C1} are present, the plurality of R_{C1} are mutually the same or different;
when a plurality of R_{C2} are present, the plurality of R_{C2} are mutually the same or different;
when a plurality of R_{C3} are present, the plurality of R_{C3} are mutually the same or different; and
a first amino group represented by a formula (C3-1) below and a second amino group represented by a formula (C3-2) below are an identical group.

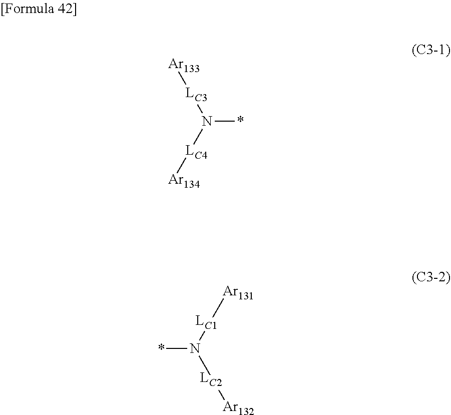

In the formulae (C3-1) and (C3-2), * each represent a bonding position to L_{C5}. In the compound represented by the formula (C3), the substituent for the “substituted or unsubstituted” group is not a group represented by —N(R_{C6})(R_{C7}), and R_{C6} and R_{C7} are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

In the compound represented by the formula (C1) in an exemplary arrangement of the organic EL device of the exemplary embodiment, the substituent for the “substituted or unsubstituted” group is not a group represented by —N(R_{C6})(R_{C7}), and R_{C6} and R_{C7} are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

In an exemplary arrangement of the organic EL device of the exemplary embodiment, the compound represented by the formula (C1) is at least one compound selected from the group consisting of a compound represented by a formula (cHT3-1), a compound represented by a formula (cHT3-2), a compound represented by a formula (cHT3-3), and a compound represented by a formula (cHT3-4) below.

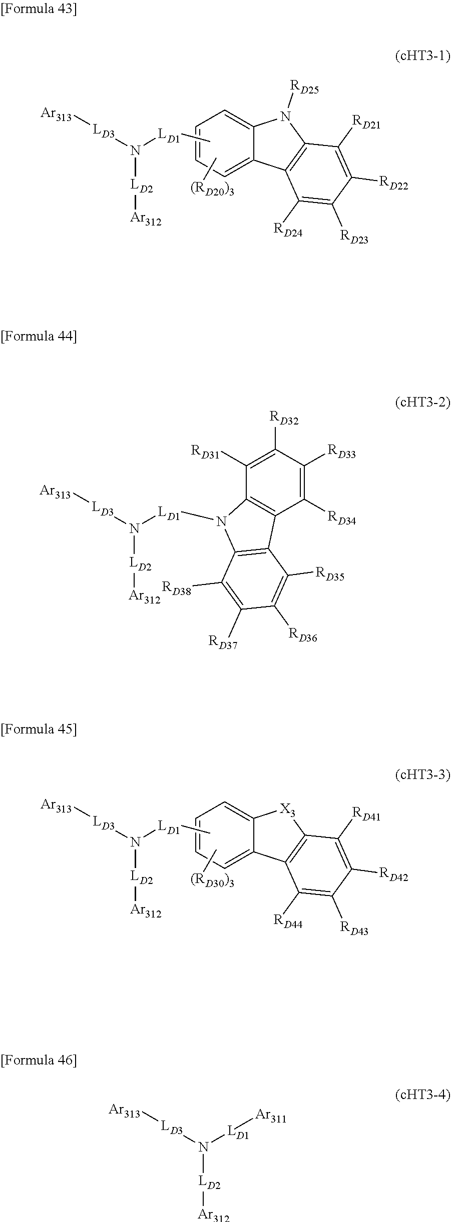

In the formulae (cHT3-1), (cHT3-2), (cHT3-3), and (cHT3-4):
Ar₃₁₂, Ar₃₁₃, L_{D1}, L_{D2}, and L_{D3} each independently represent the same as Ar₃₁₂, Ar₃₁₃, L_{D1}, L_{D2}, and L_{D3} in the formula (C1);
Ar₃₁₁ is a group represented by one of formulae (1-a), (1-b), (1-c), and (1-d) below;
at least one combination of adjacent two or more of R_{D20} to R_{D24} are mutually bonded to form a substituted or unsubstituted monocyclic ring, mutually bonded to form a substituted or unsubstituted fused ring, or not mutually bonded;
at least one combination of adjacent two or more of R_{D31} to R_{D38} are mutually bonded to form a substituted or unsubstituted monocyclic ring, mutually bonded to form a substituted or unsubstituted fused ring, or not mutually bonded;
at least one combination of adjacent two or more of R_{D40} to R_{D44} are mutually bonded to form a substituted or unsubstituted monocyclic ring, mutually bonded to form a substituted or unsubstituted fused ring, or not mutually bonded;
X₃ is an oxygen atom, a sulfur atom, or C(R_{D45})(R_{D46});
a combination of R_{D45} and R_{D46} are mutually bonded to form a substituted or unsubstituted monocyclic ring, mutually bonded to form a substituted or unsubstituted fused ring, or not mutually bonded;
R_{D25}, and R_{D20} to R_{D24}, R_{D31} to R_{D38}, R_{D40} to R_{D44}, R_{D45} and R_{D46} forming neither the substituted or unsubstituted monocyclic ring nor the substituted or unsubstituted fused ring are each independently a hydrogen atom, a cyano group, a substituted or unsubstituted alkyl group having 1 to 50 carbon atoms, a substituted or unsubstituted alkyl halide group having 1 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a substituted or unsubstituted aryl group having 6 to 50 ring carbon atoms, or a substituted or unsubstituted heterocyclic group having 5 to 50 ring atoms;
in the compounds represented by the formulae (cHT3-1), (cHT3-2), (cHT3-3), and (cHT3-4), R₉₀₁ to R₉₀₄ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different; and
when a plurality of R₉₀₄ are present, the plurality of R₉₀₄ are mutually the same or different.

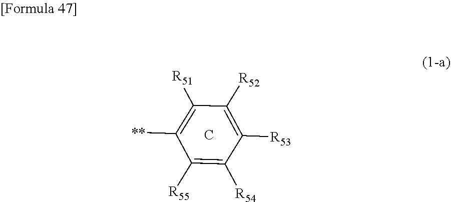

In the formula (1-a):
none of a combination(s) of adjacent two or more of R₅₁ to R₅₅ are bonded to each other;
R₅₁ to R₅₅ are each independently a hydrogen atom or a substituted or unsubstituted alkyl group having 1 to 6 carbon atoms; and
** represents a bonding position to L_{D1}.

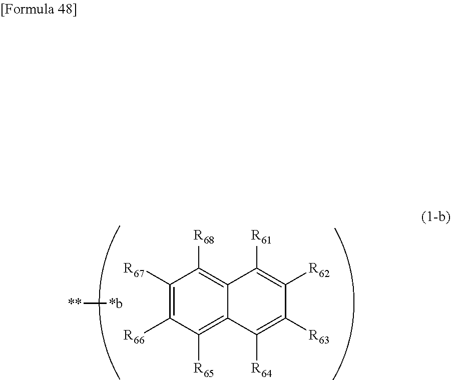

In the formula (1-b):
one of R₆₁ to R₆₈ is a single bond with *b;
none of a combination(s) of adjacent two or more of R₆₁ to R₆₈ not being the single bond with *b are bonded to each other;
R₆₁ to R₆₈ not being the single bond with *b are each independently a hydrogen atom, a substituted or unsubstituted alkyl group having 1 to 6 carbon atoms, or a substituted or unsubstituted aryl group having 6 to 12 ring carbon atoms; and
** represents a bonding position to L_{D1}.

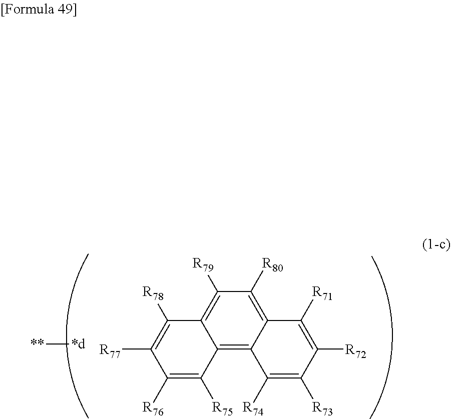

In the formula (1-c):
one of R₇₁ to R₈₀ is a single bond with *d;
none of a combination(s) of adjacent two or more of R₇₁ to R₈₀ not being the single bond with *d are bonded to each other;
R₇₁ to R₈₀ not being the single bond with *d are each independently a hydrogen atom, a substituted or unsubstituted alkyl group having 1 to 6 carbon atoms, or a substituted or unsubstituted aryl group having 6 to 12 ring carbon atoms; and
** represents a bonding position to L_{D1}.

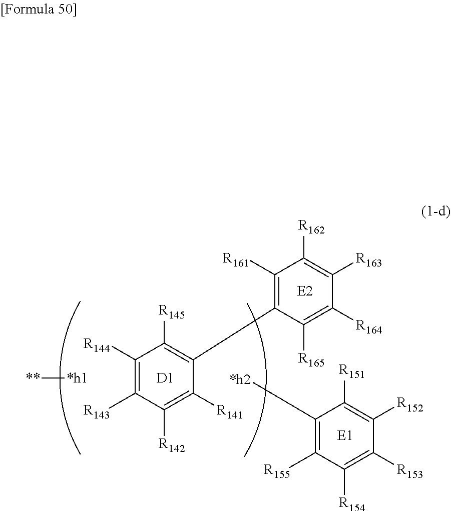

In the formula (1-d):
one of R₁₄₁ to R₁₄₅ is a single bond with *h1, and another one of R₁₄₁ to R₁₄₅ is a single bond with *h2;
none of a combination(s) of adjacent two or more of R₁₄₁ to R₁₄₅ not being the single bond with *h1 and not being the single bond with *h2 are bonded to each other;
at least one combination of adjacent two or more of R₁₅₁ to R₁₅₅ are mutually bonded to form a substituted or unsubstituted monocyclic ring, mutually bonded to form a substituted or unsubstituted fused ring, or not mutually bonded;
at least one combination of adjacent two or more of R₁₆₁ to R₁₆₅ are mutually bonded to form a substituted or unsubstituted monocyclic ring, mutually bonded to form a substituted or unsubstituted fused ring, or not mutually bonded; and
R₁₄₁ to R₁₄₅ not being the single bond with *h1 and not being the single bond with *h2 as well as R₁₅₁ to R₁₅₅ and R₁₆₁ to R₁₆₅ forming neither the substituted or unsubstituted monocyclic ring nor the substituted or unsubstituted fused ring are each independently a hydrogen atom, a substituted or unsubstituted alkyl group having 1 to 6 carbon atoms, or a substituted or unsubstituted aryl group having 6 to 12 ring carbon atoms; and ** represents a bonding position to L_{D1}.

In an exemplary arrangement of the organic EL device of the exemplary embodiment, in the compounds represented by the formulae (cHT3-1), (cHT3-2), (cHT3-3), and (cHT3-4), the substituent for the “substituted or unsubstituted” group is not a group represented by —N(R_{C6})(R_{C7}), and R_{C6} and R_{C7} are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

In an exemplary arrangement of the organic EL device of the exemplary embodiment, the first anode side organic layer contains at least one compound selected from the group consisting of a compound represented by the formula (cHT2-1), a compound represented by the formula (cHT2-2), and a compound represented by the formula (cHT2-3), and the second anode side organic layer contains at least one compound selected from the group consisting of a compound represented by the formula (cHT3-1), a compound represented by the formula (cHT3-2), a compound represented by the formula (cHT3-3), and a compound represented by the formula (cHT3-4).

### Third Anode Side Organic Layer

In an exemplary arrangement of the organic EL device of the exemplary embodiment, the hole transporting zone further includes a third anode side organic layer disposed between the second anode side organic layer and the emitting region.

In an exemplary arrangement of the organic EL device of the exemplary embodiment, the second anode side organic layer is in direct contact with the third anode side organic layer.

In an exemplary arrangement of the organic EL device of the exemplary embodiment, the third anode side organic layer is in direct contact with the emitting region.

In an exemplary arrangement of the organic EL device of the exemplary embodiment, the hole transporting zone consists of the first anode side organic layer, the second anode side organic layer and the third anode side organic layer. In this case, the total film thickness of the hole transporting zone corresponds to a total of a film thickness of the first anode side organic layer, a film thickness of the second anode side organic layer and a film thickness of the third anode side organic layer.

In an exemplary arrangement of the organic EL device of the exemplary embodiment, all the compound(s) contained in the second anode side organic layer is/are different from all the compound(s) contained in the third anode side organic layer.

An arrangement satisfying the above condition is, for instance, as follows: when a compound AA, a compound AB and a compound BB are different compounds, the second anode side organic layer contains a single type of compound (the compound AA) and the third anode side organic layer contains a single type of compound (the compound BB). Further, for instance, the above condition is satisfied also when the second anode side organic layer contains two types of compounds (the compound AA and the compound AB) and the third anode side organic layer contains a single type of compound (the compound BB), because both the compounds AA and AB are different from the compound BB.

On the other hand, for instance, the above condition is not satisfied when the second anode side organic layer contains two types of compounds (the compound AA and the compound AB) and the third anode side organic layer contains a single type of compound (the compound AB), because the second anode side organic layer and the third anode side organic layer contain the same compound (the compound AB).

In an exemplary arrangement of the organic EL device of the exemplary embodiment, the third anode side organic layer contains no compound contained in the first anode side organic layer.

In an exemplary arrangement of the organic EL device of the exemplary embodiment, the third anode side organic layer contains at least one compound selected from the group consisting of a compound represented by the formula (cHT3-1), a compound represented by the formula (cHT3-2), a compound represented by the formula (cHT3-3), and a compound represented by the formula (cHT3-4).

In an exemplary arrangement of the organic EL device of the exemplary embodiment, the first anode side organic layer, the second anode side organic layer, and the third anode side organic layer each contain a monoamine compound having only one substituted or unsubstituted amino group in a molecule.

In an exemplary arrangement of the organic EL device of the exemplary embodiment, the first anode side organic layer, the second anode side organic layer, and the third anode side organic layer contain no diamine compound.

In an exemplary arrangement of the organic EL device of the exemplary embodiment, at least one of the first anode side organic layer, the second anode side organic layer, or the third anode side organic layer may contain a diamine compound.

In an exemplary arrangement of the organic EL device of the exemplary embodiment, the compound contained in the third anode side organic layer is occasionally referred to as a third hole transporting zone material.

### Forth Anode Side Organic Layer

In an exemplary arrangement of the organic EL device of the exemplary embodiment, the hole transporting zone further includes a fourth anode side organic layer disposed between the third anode side organic layer and the emitting region.

In an exemplary arrangement of the organic EL device of the exemplary embodiment, the fourth anode side organic layer is in direct contact with the emitting region.

In an exemplary arrangement of the organic EL device of the exemplary embodiment, the fourth anode side organic layer is in direct contact with the third anode side organic layer.

In an exemplary arrangement of the organic EL device of the exemplary embodiment, the first anode side organic layer, the second anode side organic layer, the third anode side organic layer, and the fourth anode side organic layer are disposed in this order from a side close to the anode.

In an exemplary arrangement of the organic EL device of the exemplary embodiment, the fourth anode side organic layer is a blocking layer. For instance, when the blocking layer is disposed close to the anode with respect to the first emitting layer, the blocking layer permits transport of holes and blocks electrons from reaching each organic layer in the hole transporting zone provided closer to the anode beyond the blocking layer. Alternatively, the blocking layer may be provided in direct contact with the first emitting layer so that excitation energy does not leak out from the first emitting layer toward neighboring layer(s). The blocking layer disposed close to the anode with respect to the first emitting layer blocks excitons generated in the first emitting layer from transferring to each organic layer in the hole transporting zone. The first emitting layer is preferably in direct contact with the blocking layer.

In an exemplary arrangement of the organic EL device of the exemplary embodiment, the fourth anode side organic layer contains a fourth hole transporting zone material.

In an exemplary arrangement of the organic EL device of the exemplary embodiment, the fourth hole transporting zone material and the third hole transporting zone material are different compounds.

In an exemplary arrangement of the organic EL device of the exemplary embodiment, the fourth hole transporting zone material, the third hole transporting zone material, and the second hole transporting zone material are different compounds.

In an exemplary arrangement of the organic EL device of the exemplary embodiment, the fourth anode side organic layer contains at least one compound selected from the group consisting of a compound represented by the formula (cHT3-1), a compound represented by the formula (cHT3-2), a compound represented by the formula (cHT3-3), and a compound represented by the formula (cHT3-4).

In an exemplary arrangement of the organic EL device of the exemplary embodiment, although the third anode side organic layer and the fourth anode side organic layer may each contain a compound represented by the formula (cHT3-1), the compound contained in the third anode side organic layer and the compound contained in the fourth anode side organic layer are mutually different in molecular structures.

In an exemplary arrangement of the organic EL device of the exemplary embodiment, the first anode side organic layer, the second anode side organic layer, the third anode side organic layer, and the fourth anode side organic layer each contain a monoamine compound having only one substituted or unsubstituted amino group in a molecule.

In an exemplary arrangement of the organic EL device of the exemplary embodiment, the first anode side organic layer, the second anode side organic layer, the third anode side organic layer, and the fourth anode side organic layer contain no diamine compound.

In an exemplary arrangement of the organic EL device of the exemplary embodiment, at least one of the first anode side organic layer, the second anode side organic layer, the third anode side organic layer, or the fourth anode side organic layer may also contain a diamine compound.

In the exemplary arrangement, the groups specified to be “substituted or unsubstituted” are each preferably an “unsubstituted” group.

In the exemplary embodiment, the first hole transporting zone material, the second hole transporting zone material, the third hole transporting zone material, and the fourth hole transporting zone material each may be occasionally referred to as a hole transporting zone material.

### Method of Producing Hole Transporting Zone Material

The hole transporting zone material according to the exemplary embodiment can be produced by a known method or through a known alternative reaction using a known material(s) tailored for the target compound in accordance with the known method.

### Specific Examples of Hole Transporting Zone Material

Specific examples of the hole transporting zone material according to the exemplary embodiment include the following compounds. However, the invention is by no means limited to the specific examples.

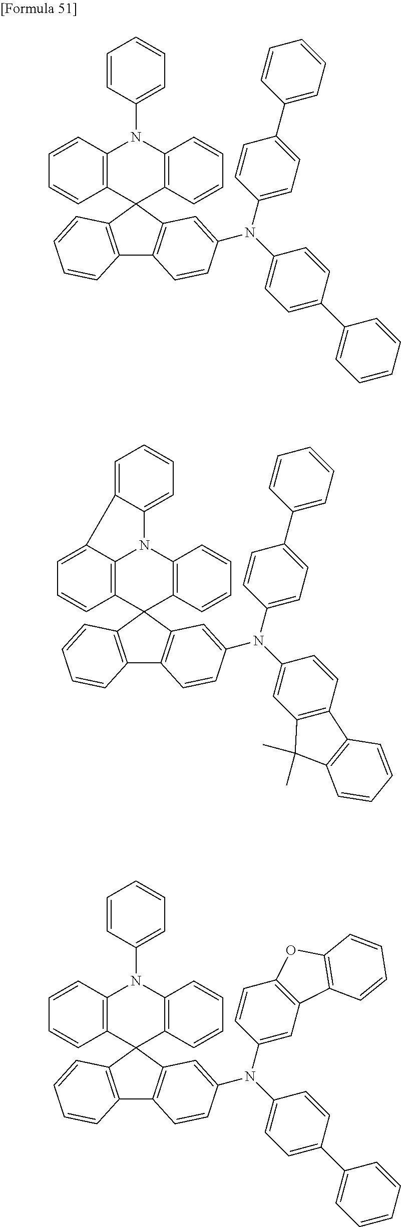 [SMILES: c1ccc(-c2ccc(N(c3ccc(-c4ccccc4)cc3)c3ccc4c(c3)C3(c5ccccc5-4)c4ccccc4N(c4ccccc4)c4ccccc43)cc2)cc1 ; CC1(C)c2ccccc2-c2ccc(N(c3ccc(-c4ccccc4)cc3)c3ccc4c(c3)C3(c5ccccc5-4)c4ccccc4-n4c5ccccc5c5cccc3c54)cc21 ; c1ccc(-c2ccc(N(c3ccc4c(c3)C3(c5ccccc5-4)c4ccccc4N(c4ccccc4)c4ccccc43)c3ccc4oc5ccccc5c4c3)cc2)cc1]

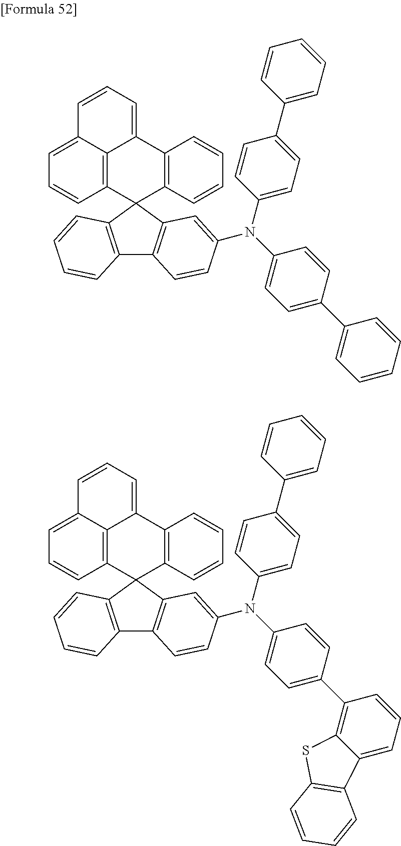 [SMILES: c1ccc(-c2ccc(N(c3ccc(-c4ccccc4)cc3)c3ccc4c(c3)C3(c5ccccc5-4)c4ccccc4-c4cccc5cccc3c45)cc2)cc1 ; c1ccc(-c2ccc(N(c3ccc(-c4cccc5c4sc4ccccc45)cc3)c3ccc4c(c3)C3(c5ccccc5-4)c4ccccc4-c4cccc5cccc3c45)cc2)cc1]

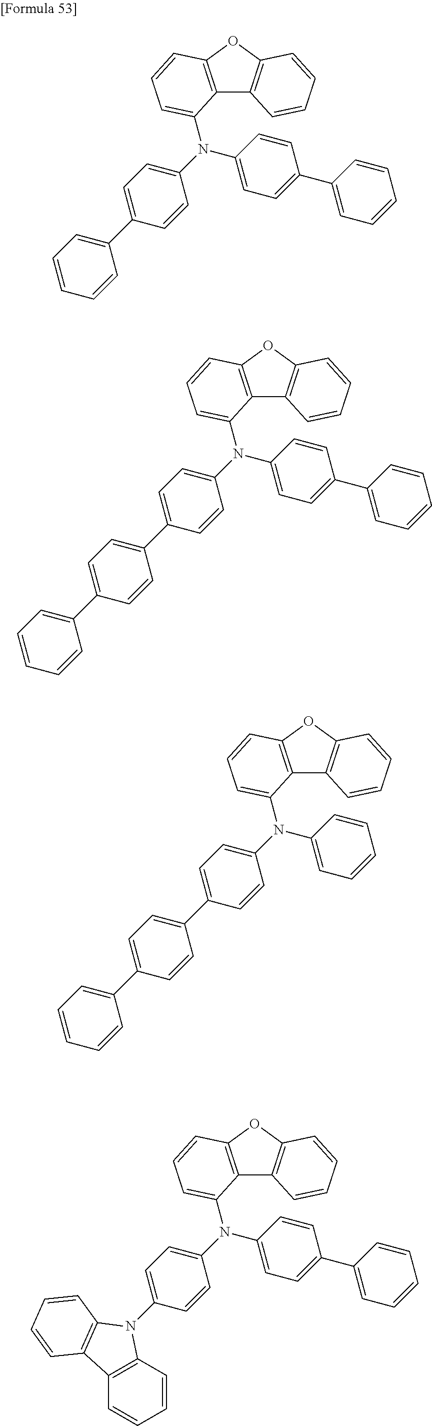 [SMILES: c1ccc(-c2ccc(N(c3ccc(-c4ccccc4)cc3)c3cccc4oc5ccccc5c34)cc2)cc1 ; c1ccc(-c2ccc(-c3ccc(N(c4ccc(-c5ccccc5)cc4)c4cccc5oc6ccccc6c45)cc3)cc2)cc1 ; c1ccc(-c2ccc(-c3ccc(N(c4ccccc4)c4cccc5oc6ccccc6c45)cc3)cc2)cc1 ; c1ccc(-c2ccc(N(c3ccc(-n4c5ccccc5c5ccccc54)cc3)c3cccc4oc5ccccc5c34)cc2)cc1]

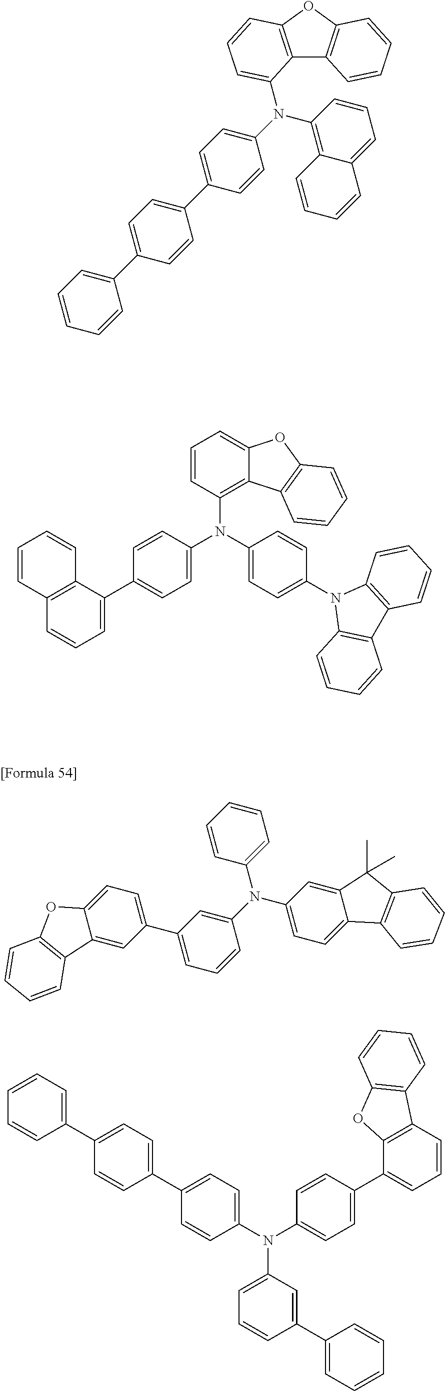 [SMILES: c1ccc(-c2ccc(-c3ccc(N(c4cccc5ccccc45)c4cccc5oc6ccccc6c45)cc3)cc2)cc1 ; c1ccc2c(-c3ccc(N(c4ccc(-n5c6ccccc6c6ccccc65)cc4)c4cccc5oc6ccccc6c45)cc3)cccc2c1 ; CC1(C)c2ccccc2-c2ccc(N(c3ccccc3)c3cccc(-c4ccc5oc6ccccc6c5c4)c3)cc21 ; c1ccc(-c2ccc(-c3ccc(N(c4ccc(-c5cccc6c5oc5ccccc56)cc4)c4cccc(-c5ccccc5)c4)cc3)cc2)cc1]

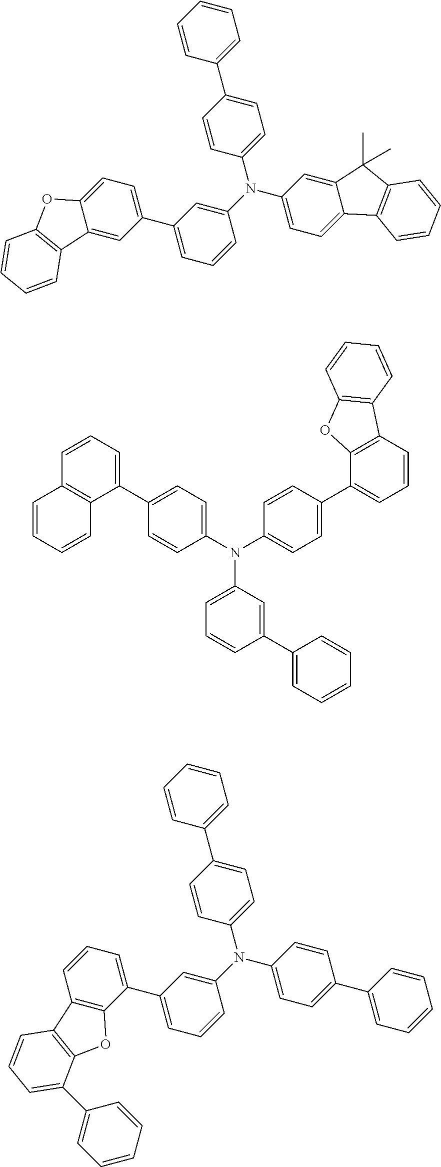 [SMILES: CC1(C)c2ccccc2-c2ccc(N(c3ccc(-c4ccccc4)cc3)c3cccc(-c4ccc5oc6ccccc6c5c4)c3)cc21 ; c1ccc(-c2cccc(N(c3ccc(-c4cccc5ccccc45)cc3)c3ccc(-c4cccc5c4oc4ccccc45)cc3)c2)cc1 ; c1ccc(-c2ccc(N(c3ccc(-c4ccccc4)cc3)c3cccc(-c4cccc5c4oc4c(-c6ccccc6)cccc45)c3)cc2)cc1]

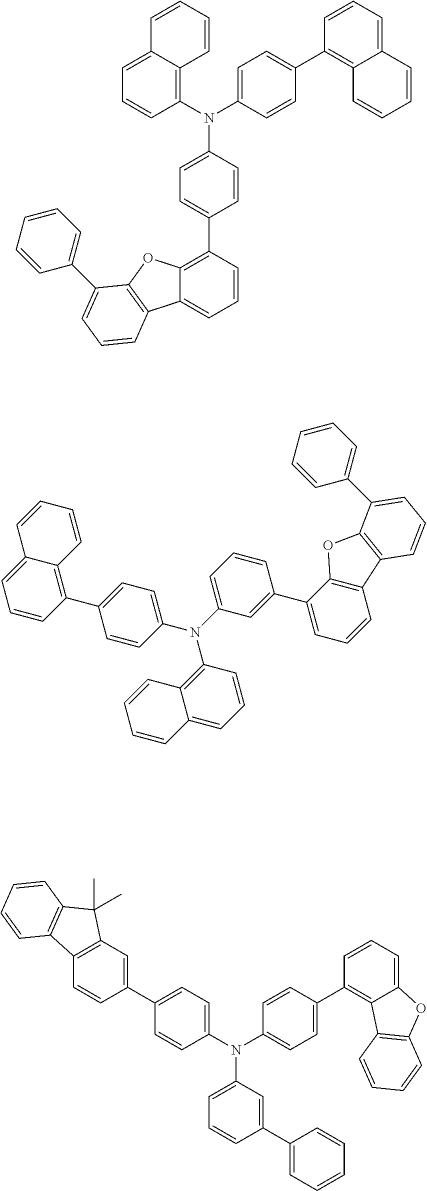 [SMILES: c1ccc(-c2cccc3c2oc2c(-c4ccc(N(c5ccc(-c6cccc7ccccc67)cc5)c5cccc6ccccc56)cc4)cccc23)cc1 ; c1ccc(-c2cccc3c2oc2c(-c4cccc(N(c5ccc(-c6cccc7ccccc67)cc5)c5cccc6ccccc56)c4)cccc23)cc1 ; CC1(C)c2ccccc2-c2ccc(-c3ccc(N(c4ccc(-c5cccc6oc7ccccc7c56)cc4)c4cccc(-c5ccccc5)c4)cc3)cc21]

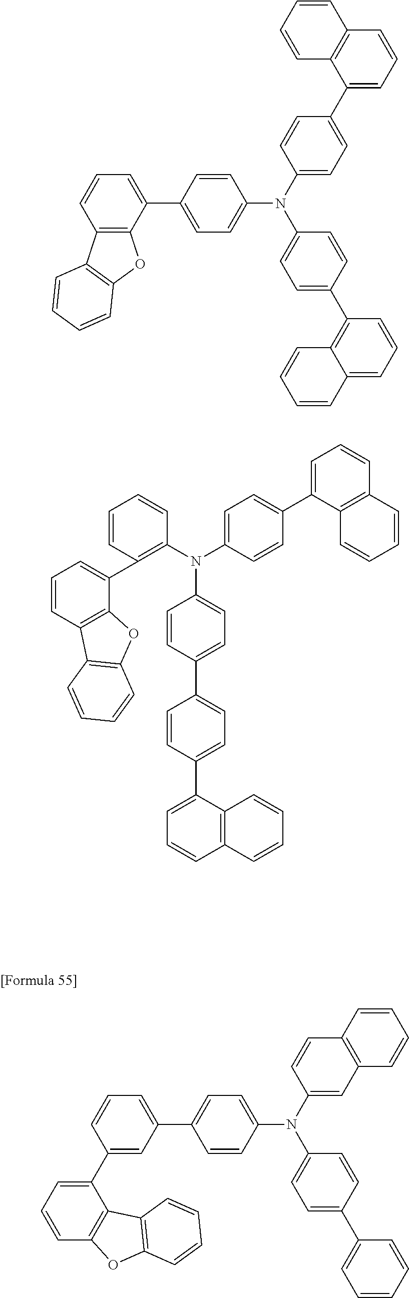

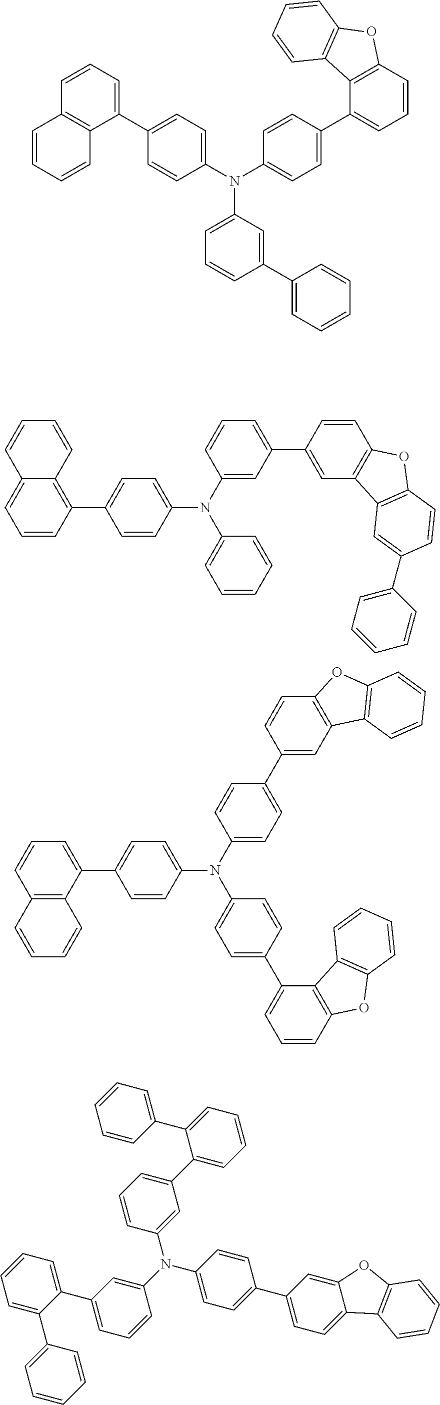

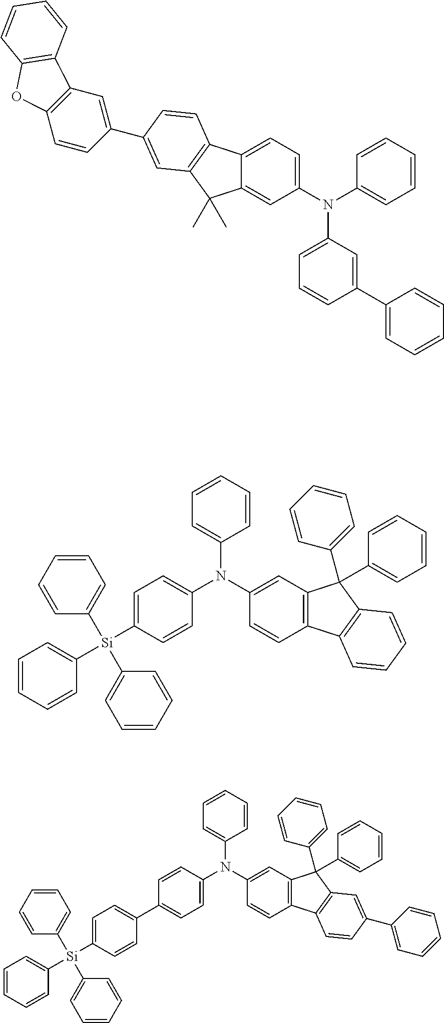 [SMILES: CC1(C)c2cc(-c3ccc4oc5ccccc5c4c3)ccc2-c2ccc(N(c3ccccc3)c3cccc(-c4ccccc4)c3)cc21 ; c1ccc(N(c2ccc([Si](c3ccccc3)(c3ccccc3)c3ccccc3)cc2)c2ccc3c(c2)C(c2ccccc2)(c2ccccc2)c2ccccc2-3)cc1 ; c1ccc(-c2ccc3c(c2)C(c2ccccc2)(c2ccccc2)c2cc(N(c4ccccc4)c4ccc(-c5ccc([Si](c6ccccc6)(c6ccccc6)c6ccccc6)cc5)cc4)ccc2-3)cc1]

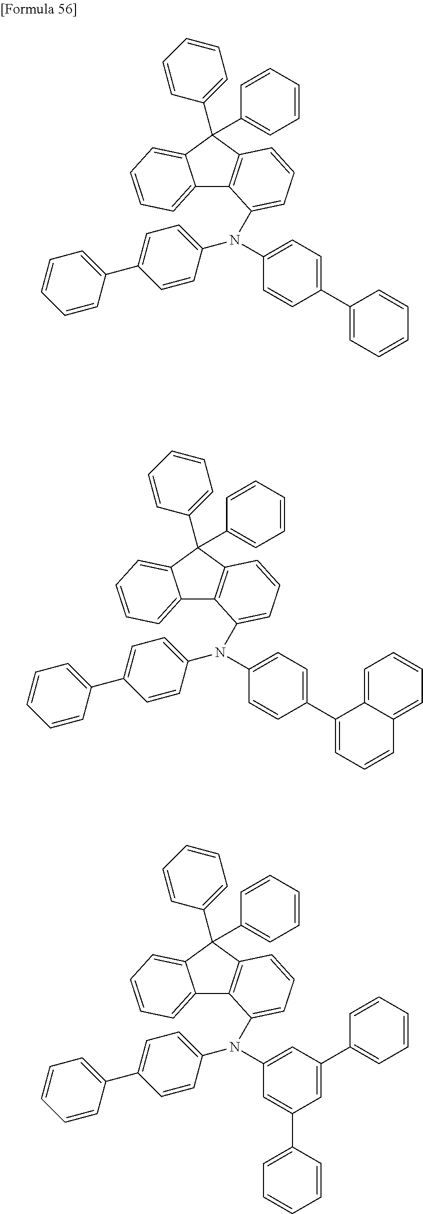 [SMILES: c1ccc(-c2ccc(N(c3ccc(-c4ccccc4)cc3)c3cccc4c3-c3ccccc3C4(c3ccccc3)c3ccccc3)cc2)cc1 ; c1ccc(-c2ccc(N(c3ccc(-c4cccc5ccccc45)cc3)c3cccc4c3-c3ccccc3C4(c3ccccc3)c3ccccc3)cc2)cc1 ; c1ccc(-c2ccc(N(c3cc(-c4ccccc4)cc(-c4ccccc4)c3)c3cccc4c3-c3ccccc3C4(c3ccccc3)c3ccccc3)cc2)cc1]

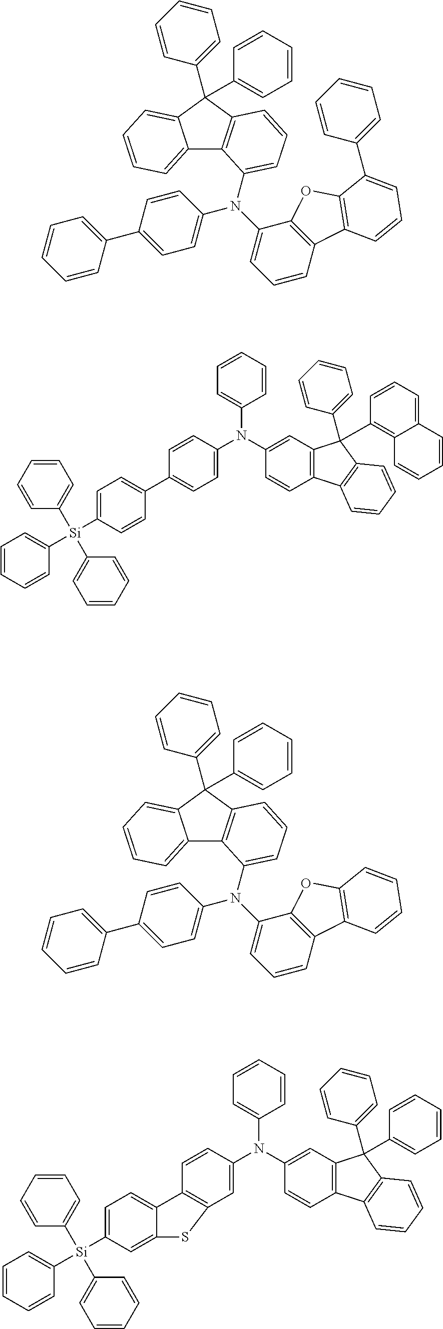 [SMILES: c1ccc(-c2ccc(N(c3cccc4c3-c3ccccc3C4(c3ccccc3)c3ccccc3)c3cccc4c3oc3c(-c5ccccc5)cccc34)cc2)cc1 ; c1ccc(N(c2ccc(-c3ccc([Si](c4ccccc4)(c4ccccc4)c4ccccc4)cc3)cc2)c2ccc3c(c2)C(c2ccccc2)(c2cccc4ccccc24)c2ccccc2-3)cc1 ; c1ccc(-c2ccc(N(c3cccc4c3-c3ccccc3C4(c3ccccc3)c3ccccc3)c3cccc4c3oc3ccccc34)cc2)cc1 ; c1ccc(N(c2ccc3c(c2)C(c2ccccc2)(c2ccccc2)c2ccccc2-3)c2ccc3c(c2)sc2cc([Si](c4ccccc4)(c4ccccc4)c4ccccc4)ccc23)cc1]

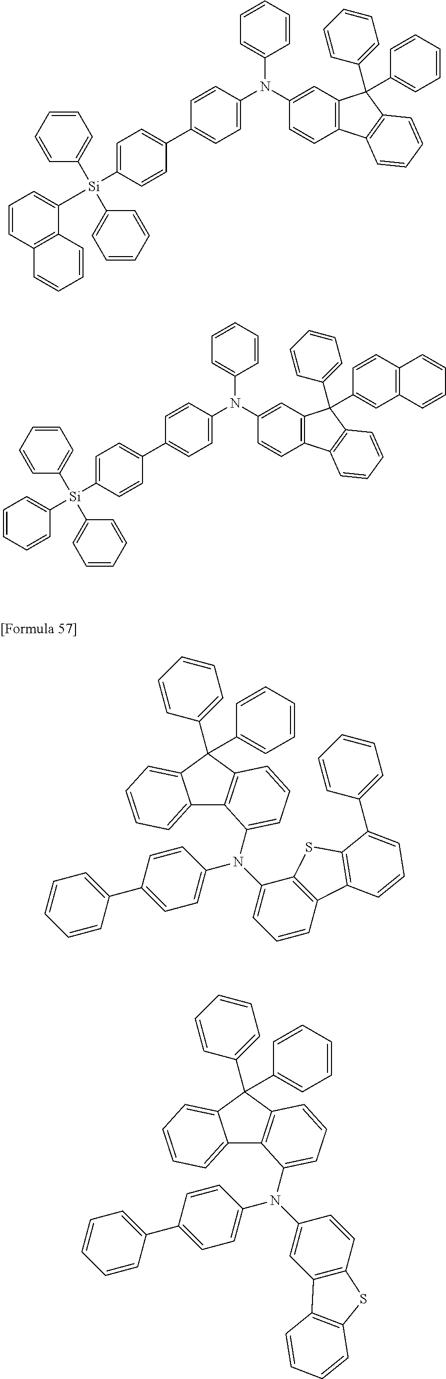 [SMILES: c1ccc(N(c2ccc(-c3ccc([Si](c4ccccc4)(c4ccccc4)c4cccc5ccccc45)cc3)cc2)c2ccc3c(c2)C(c2ccccc2)(c2ccccc2)c2ccccc2-3)cc1 ; c1ccc(N(c2ccc(-c3ccc([Si](c4ccccc4)(c4ccccc4)c4ccccc4)cc3)cc2)c2ccc3c(c2)C(c2ccccc2)(c2ccc4ccccc4c2)c2ccccc2-3)cc1 ; c1ccc(-c2ccc(N(c3cccc4c3-c3ccccc3C4(c3ccccc3)c3ccccc3)c3cccc4c3sc3c(-c5ccccc5)cccc34)cc2)cc1 ; c1ccc(-c2ccc(N(c3ccc4sc5ccccc5c4c3)c3cccc4c3-c3ccccc3C4(c3ccccc3)c3ccccc3)cc2)cc1]

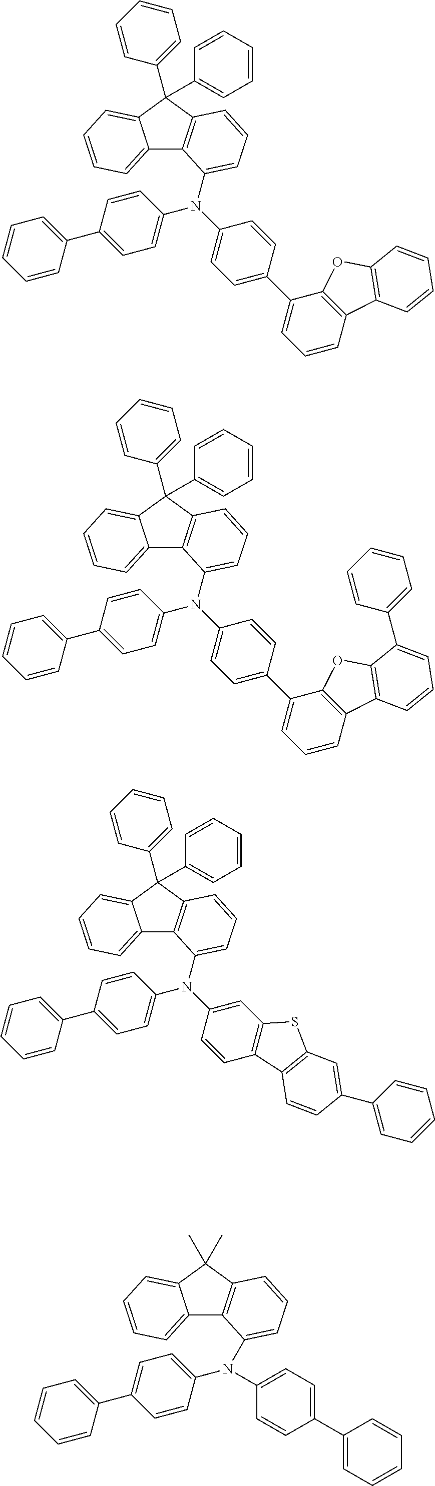 [SMILES: c1ccc(-c2ccc(N(c3ccc(-c4cccc5c4oc4ccccc45)cc3)c3cccc4c3-c3ccccc3C4(c3ccccc3)c3ccccc3)cc2)cc1 ; c1ccc(-c2ccc(N(c3ccc(-c4cccc5c4oc4c(-c6ccccc6)cccc45)cc3)c3cccc4c3-c3ccccc3C4(c3ccccc3)c3ccccc3)cc2)cc1 ; c1ccc(-c2ccc(N(c3ccc4c(c3)sc3cc(-c5ccccc5)ccc34)c3cccc4c3-c3ccccc3C4(c3ccccc3)c3ccccc3)cc2)cc1 ; CC1(C)c2ccccc2-c2c(N(c3ccc(-c4ccccc4)cc3)c3ccc(-c4ccccc4)cc3)cccc21]

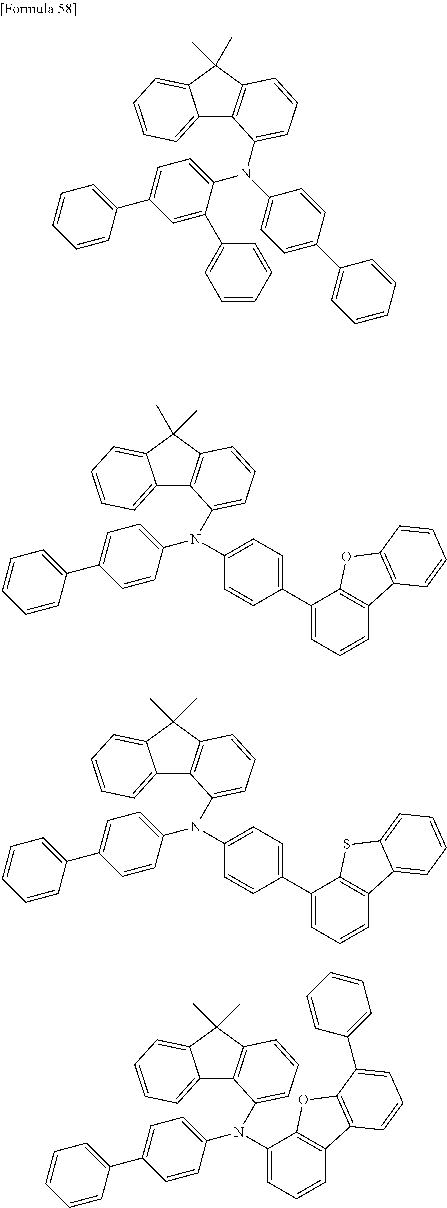

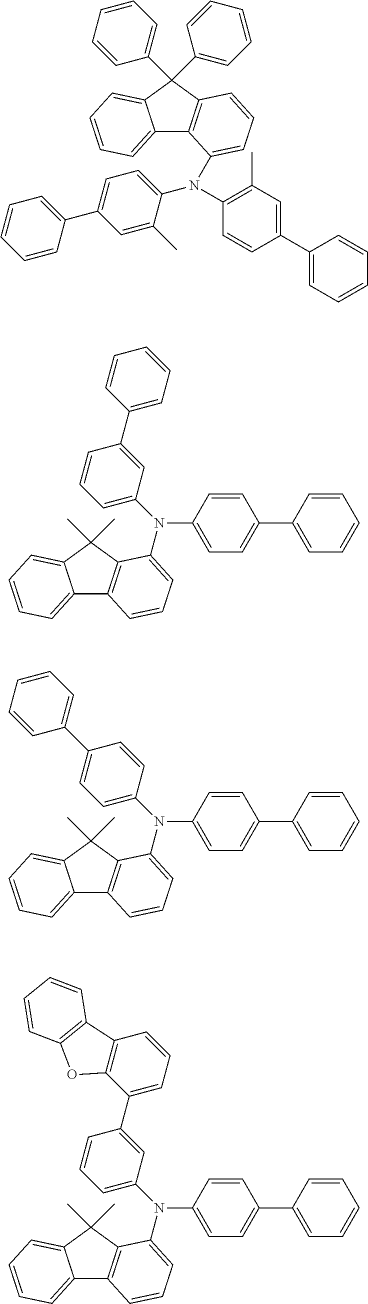 [SMILES: Cc1cc(-c2ccccc2)ccc1N(c1ccc(-c2ccccc2)cc1C)c1cccc2c1-c1ccccc1C2(c1ccccc1)c1ccccc1 ; CC1(C)c2ccccc2-c2cccc(N(c3ccc(-c4ccccc4)cc3)c3cccc(-c4ccccc4)c3)c21 ; CC1(C)c2ccccc2-c2cccc(N(c3ccc(-c4ccccc4)cc3)c3ccc(-c4ccccc4)cc3)c21 ; CC1(C)c2ccccc2-c2cccc(N(c3ccc(-c4ccccc4)cc3)c3cccc(-c4cccc5c4oc4ccccc45)c3)c21]

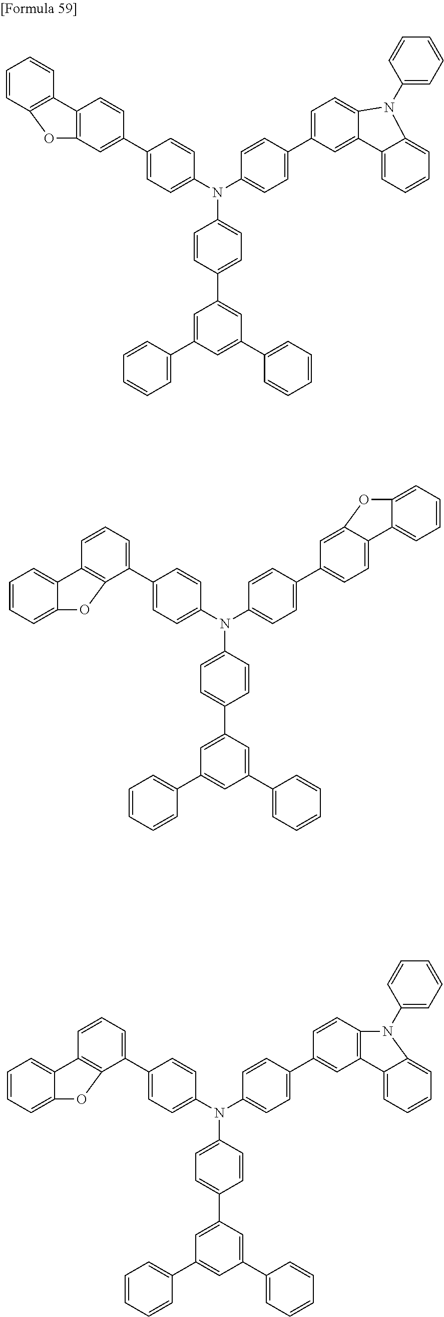 [SMILES: c1ccc(-c2cc(-c3ccccc3)cc(-c3ccc(N(c4ccc(-c5ccc6c(c5)oc5ccccc56)cc4)c4ccc(-c5ccc6c(c5)c5ccccc5n6-c5ccccc5)cc4)cc3)c2)cc1 ; c1ccc(-c2cc(-c3ccccc3)cc(-c3ccc(N(c4ccc(-c5ccc6c(c5)oc5ccccc56)cc4)c4ccc(-c5cccc6c5oc5ccccc56)cc4)cc3)c2)cc1 ; c1ccc(-c2cc(-c3ccccc3)cc(-c3ccc(N(c4ccc(-c5ccc6c(c5)c5ccccc5n6-c5ccccc5)cc4)c4ccc(-c5cccc6c5oc5ccccc56)cc4)cc3)c2)cc1]

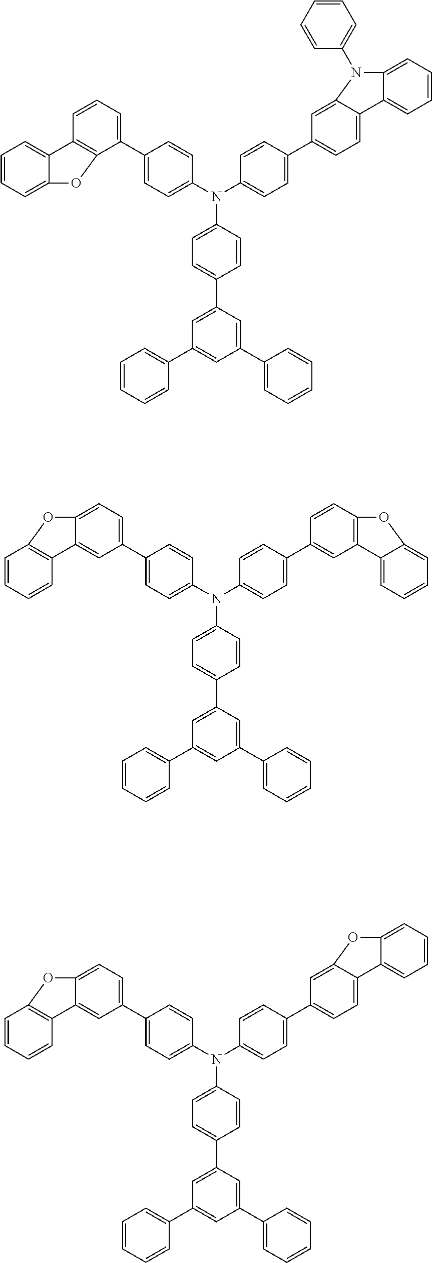 [SMILES: c1ccc(-c2cc(-c3ccccc3)cc(-c3ccc(N(c4ccc(-c5ccc6c7ccccc7n(-c7ccccc7)c6c5)cc4)c4ccc(-c5cccc6c5oc5ccccc56)cc4)cc3)c2)cc1 ; c1ccc(-c2cc(-c3ccccc3)cc(-c3ccc(N(c4ccc(-c5ccc6oc7ccccc7c6c5)cc4)c4ccc(-c5ccc6oc7ccccc7c6c5)cc4)cc3)c2)cc1 ; c1ccc(-c2cc(-c3ccccc3)cc(-c3ccc(N(c4ccc(-c5ccc6c(c5)oc5ccccc56)cc4)c4ccc(-c5ccc6oc7ccccc7c6c5)cc4)cc3)c2)cc1]

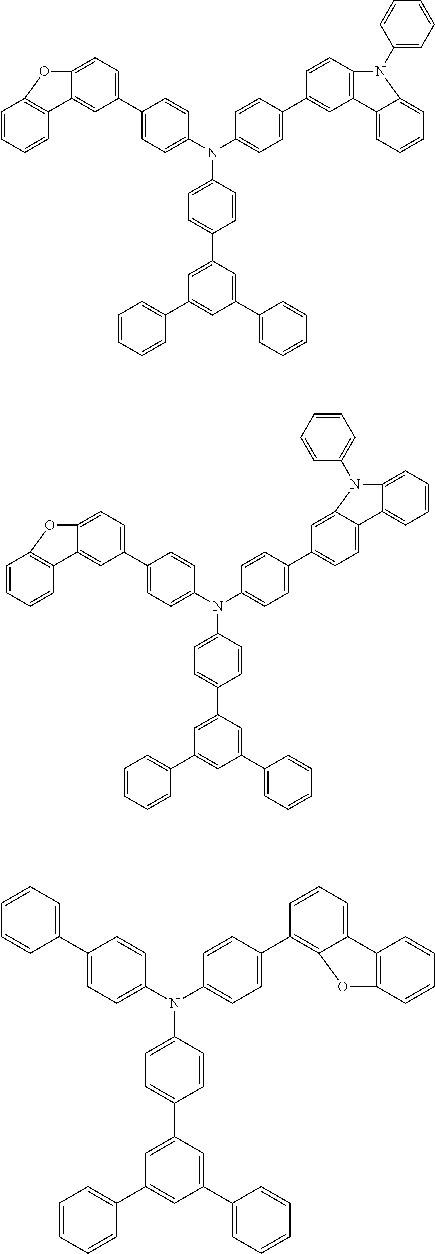 [SMILES: c1ccc(-c2cc(-c3ccccc3)cc(-c3ccc(N(c4ccc(-c5ccc6oc7ccccc7c6c5)cc4)c4ccc(-c5ccc6c(c5)c5ccccc5n6-c5ccccc5)cc4)cc3)c2)cc1 ; c1ccc(-c2cc(-c3ccccc3)cc(-c3ccc(N(c4ccc(-c5ccc6oc7ccccc7c6c5)cc4)c4ccc(-c5ccc6c7ccccc7n(-c7ccccc7)c6c5)cc4)cc3)c2)cc1 ; c1ccc(-c2ccc(N(c3ccc(-c4cc(-c5ccccc5)cc(-c5ccccc5)c4)cc3)c3ccc(-c4cccc5c4oc4ccccc45)cc3)cc2)cc1]

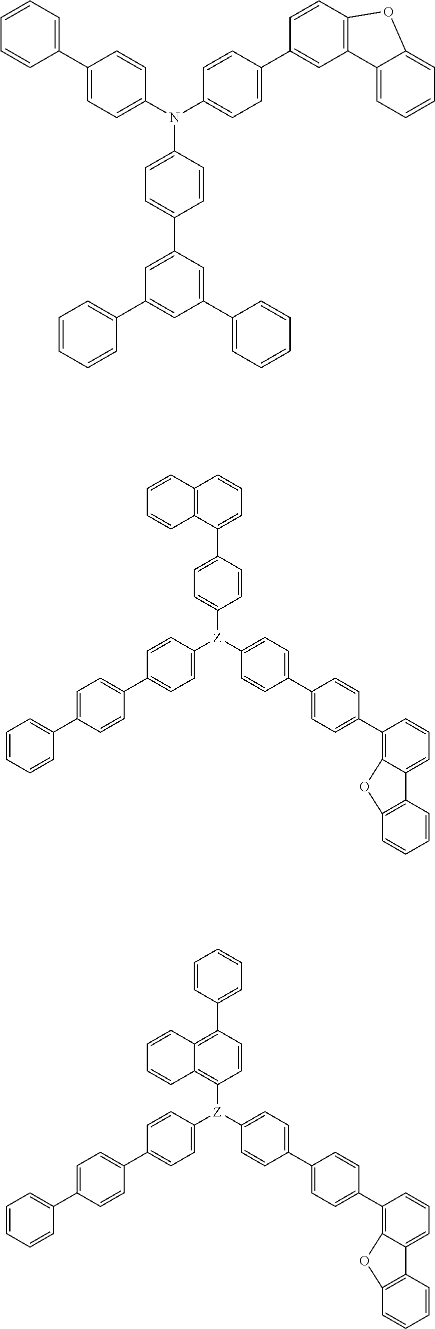

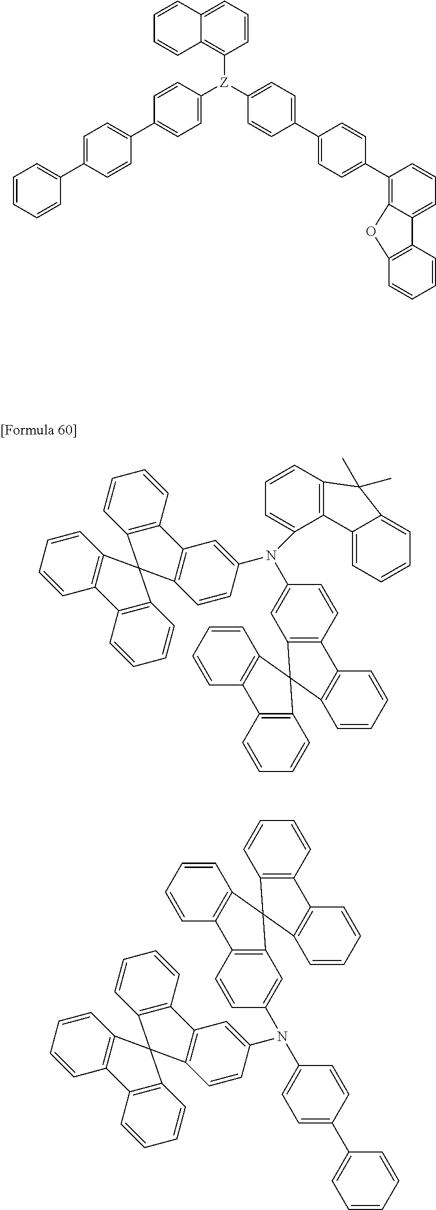

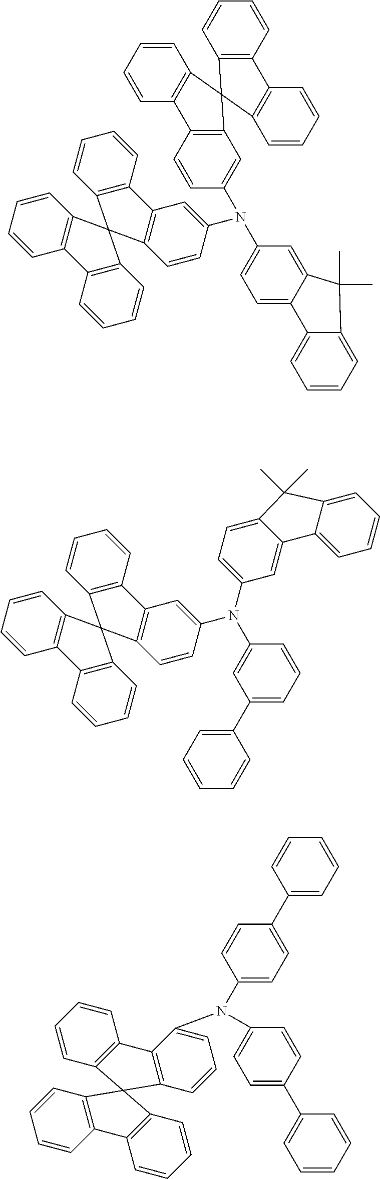 [SMILES: CC1(C)c2ccccc2-c2ccc(N(c3ccc4c(c3)-c3ccccc3C43c4ccccc4-c4ccccc43)c3ccc4c(c3)C3(c5ccccc5-c5ccccc53)c3ccccc3-4)cc21 ; CC1(C)c2ccccc2-c2cc(N(c3cccc(-c4ccccc4)c3)c3ccc4c(c3)-c3ccccc3C43c4ccccc4-c4ccccc43)ccc21 ; c1ccc(-c2ccc(N(c3ccc(-c4ccccc4)cc3)c3cccc4c3-c3ccccc3C43c4ccccc4-c4ccccc43)cc2)cc1]

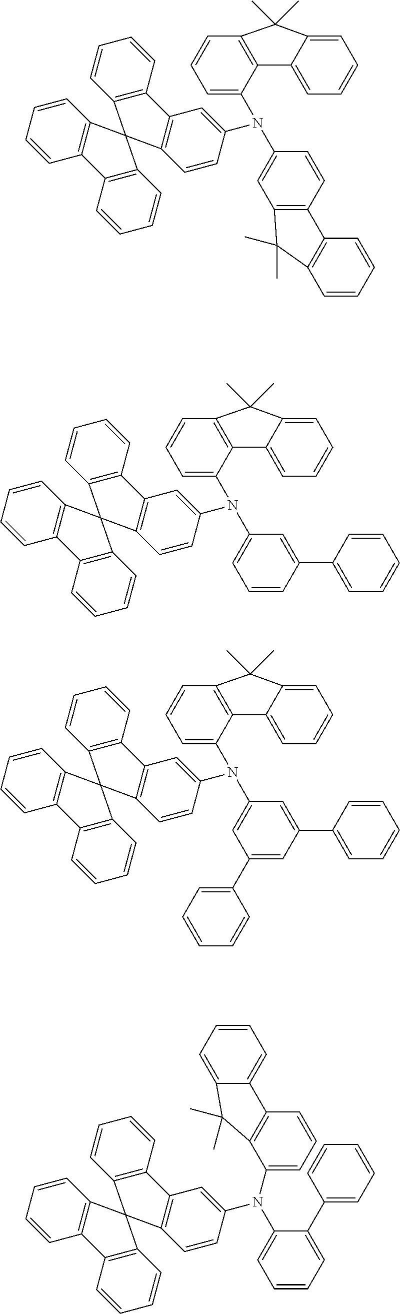 [SMILES: CC1(C)c2ccccc2-c2ccc(N(c3ccc4c(c3)-c3ccccc3C43c4ccccc4-c4ccccc43)c3cccc4c3-c3ccccc3C4(C)C)cc21 ; CC1(C)c2ccccc2-c2c(N(c3cccc(-c4ccccc4)c3)c3ccc4c(c3)-c3ccccc3C43c4ccccc4-c4ccccc43)cccc21 ; CC1(C)c2ccccc2-c2c(N(c3cc(-c4ccccc4)cc(-c4ccccc4)c3)c3ccc4c(c3)-c3ccccc3C43c4ccccc4-c4ccccc43)cccc21 ; CC1(C)c2ccccc2-c2cccc(N(c3ccc4c(c3)-c3ccccc3C43c4ccccc4-c4ccccc43)c3ccccc3-c3ccccc3)c21]

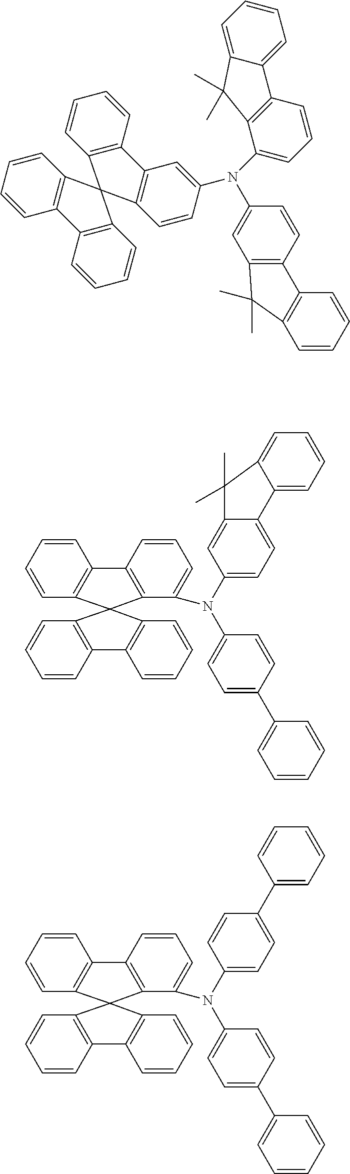 [SMILES: CC1(C)c2ccccc2-c2ccc(N(c3ccc4c(c3)-c3ccccc3C43c4ccccc4-c4ccccc43)c3cccc4c3C(C)(C)c3ccccc3-4)cc21 ; CC1(C)c2ccccc2-c2ccc(N(c3ccc(-c4ccccc4)cc3)c3cccc4c3C3(c5ccccc5-c5ccccc53)c3ccccc3-4)cc21 ; c1ccc(-c2ccc(N(c3ccc(-c4ccccc4)cc3)c3cccc4c3C3(c5ccccc5-c5ccccc53)c3ccccc3-4)cc2)cc1]

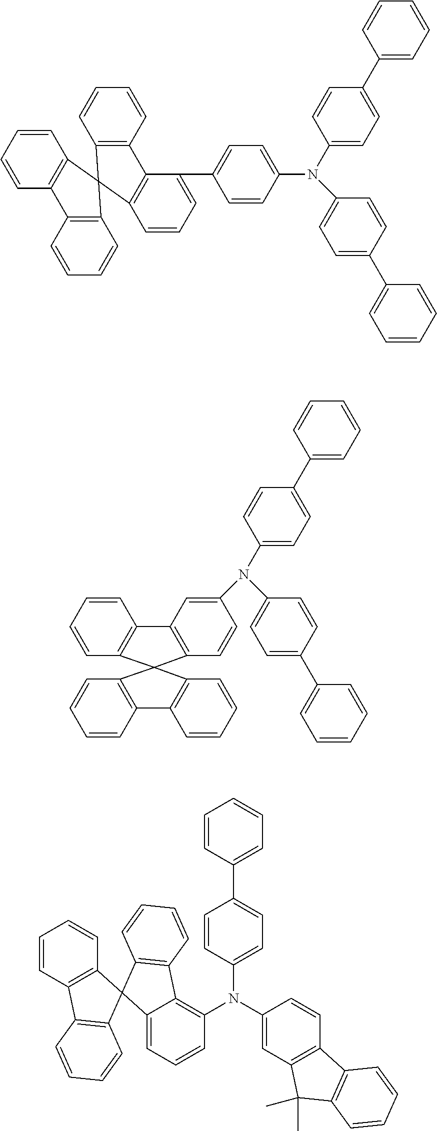 [SMILES: c1ccc(-c2ccc(N(c3ccc(-c4ccccc4)cc3)c3ccc(-c4cccc5c4-c4ccccc4C54c5ccccc5-c5ccccc54)cc3)cc2)cc1 ; c1ccc(-c2ccc(N(c3ccc(-c4ccccc4)cc3)c3ccc4c(c3)-c3ccccc3C43c4ccccc4-c4ccccc43)cc2)cc1 ; CC1(C)c2ccccc2-c2ccc(N(c3ccc(-c4ccccc4)cc3)c3cccc4c3-c3ccccc3C43c4ccccc4-c4ccccc43)cc21]

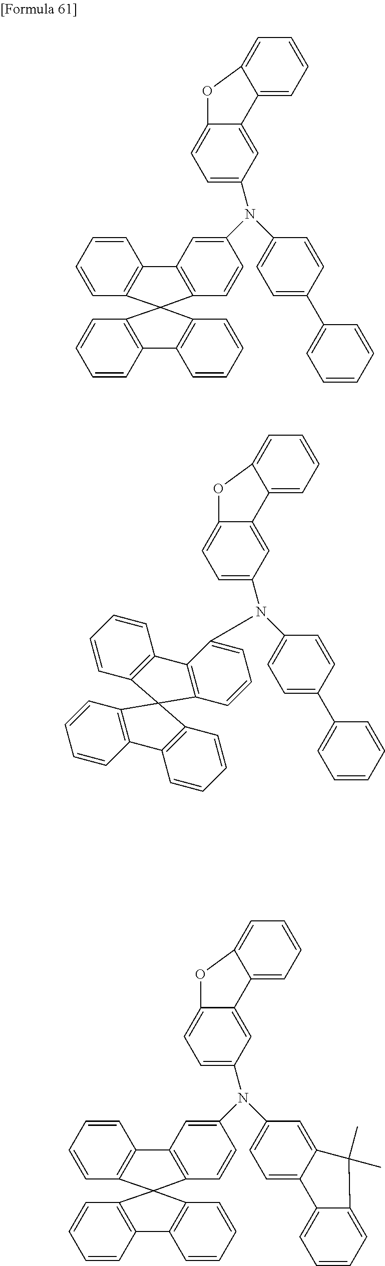 [SMILES: c1ccc(-c2ccc(N(c3ccc4c(c3)-c3ccccc3C43c4ccccc4-c4ccccc43)c3ccc4oc5ccccc5c4c3)cc2)cc1 ; c1ccc(-c2ccc(N(c3ccc4oc5ccccc5c4c3)c3cccc4c3-c3ccccc3C43c4ccccc4-c4ccccc43)cc2)cc1 ; CC1(C)c2ccccc2-c2ccc(N(c3ccc4c(c3)-c3ccccc3C43c4ccccc4-c4ccccc43)c3ccc4oc5ccccc5c4c3)cc21]

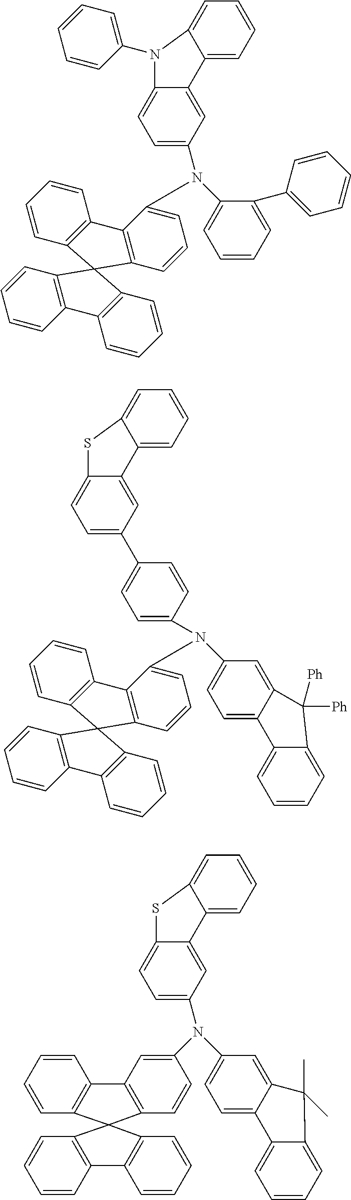

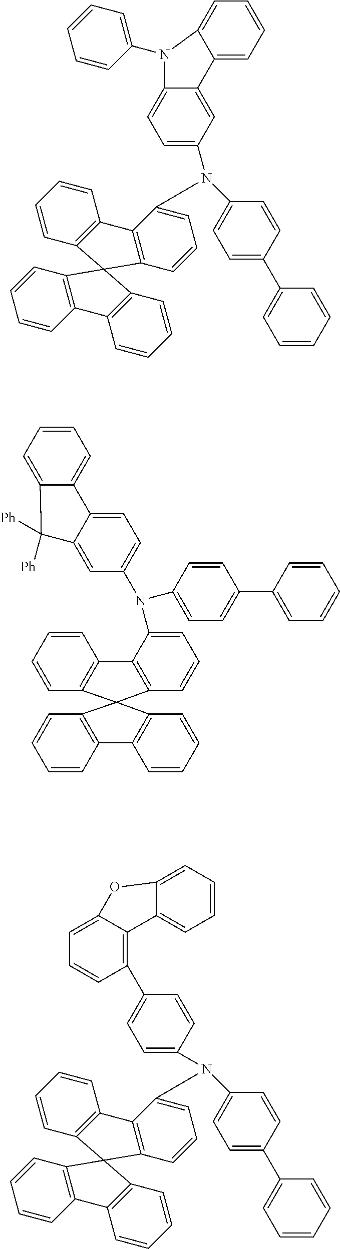 [SMILES: c1ccc(-c2ccc(N(c3ccc4c(c3)c3ccccc3n4-c3ccccc3)c3cccc4c3-c3ccccc3C43c4ccccc4-c4ccccc43)cc2)cc1 ; c1ccc(-c2ccc(N(c3ccc4c(c3)C(c3ccccc3)(c3ccccc3)c3ccccc3-4)c3cccc4c3-c3ccccc3C43c4ccccc4-c4ccccc43)cc2)cc1 ; c1ccc(-c2ccc(N(c3ccc(-c4cccc5oc6ccccc6c45)cc3)c3cccc4c3-c3ccccc3C43c4ccccc4-c4ccccc43)cc2)cc1]

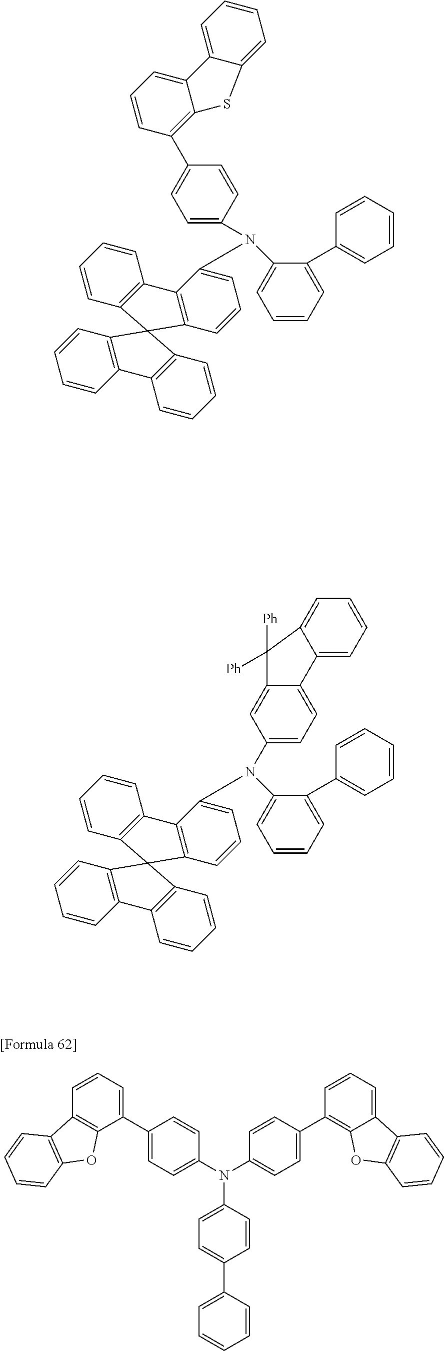 [SMILES: c1ccc(-c2ccccc2N(c2ccc(-c3cccc4c3sc3ccccc34)cc2)c2cccc3c2-c2ccccc2C32c3ccccc3-c3ccccc32)cc1 ; c1ccc(-c2ccccc2N(c2ccc3c(c2)C(c2ccccc2)(c2ccccc2)c2ccccc2-3)c2cccc3c2-c2ccccc2C32c3ccccc3-c3ccccc32)cc1 ; c1ccc(-c2ccc(N(c3ccc(-c4cccc5c4oc4ccccc45)cc3)c3ccc(-c4cccc5c4oc4ccccc45)cc3)cc2)cc1]

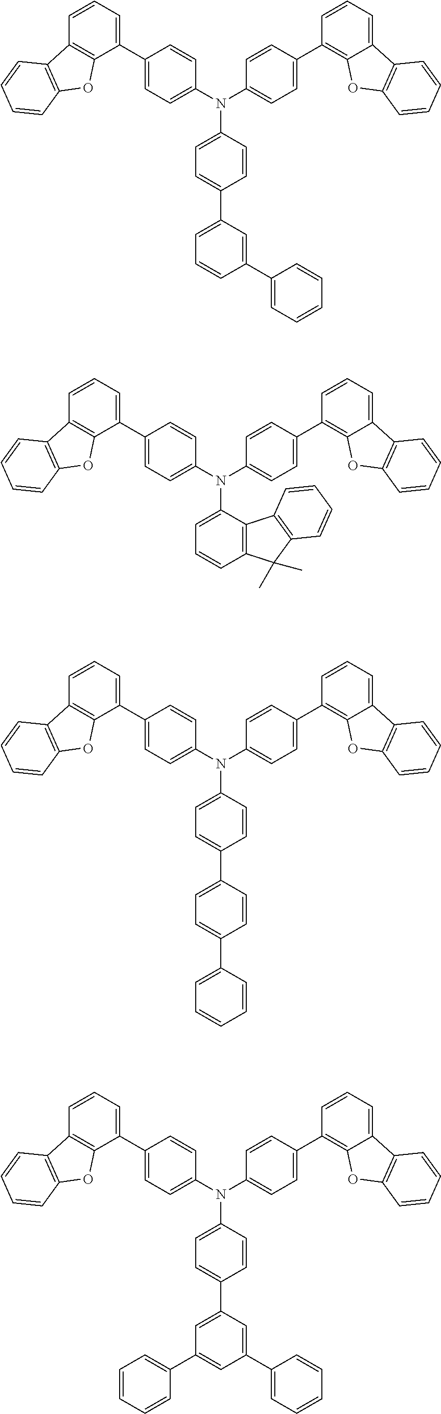 [SMILES: c1ccc(-c2cccc(-c3ccc(N(c4ccc(-c5cccc6c5oc5ccccc56)cc4)c4ccc(-c5cccc6c5oc5ccccc56)cc4)cc3)c2)cc1 ; CC1(C)c2ccccc2-c2c(N(c3ccc(-c4cccc5c4oc4ccccc45)cc3)c3ccc(-c4cccc5c4oc4ccccc45)cc3)cccc21 ; c1ccc(-c2ccc(-c3ccc(N(c4ccc(-c5cccc6c5oc5ccccc56)cc4)c4ccc(-c5cccc6c5oc5ccccc56)cc4)cc3)cc2)cc1 ; c1ccc(-c2cc(-c3ccccc3)cc(-c3ccc(N(c4ccc(-c5cccc6c5oc5ccccc56)cc4)c4ccc(-c5cccc6c5oc5ccccc56)cc4)cc3)c2)cc1]

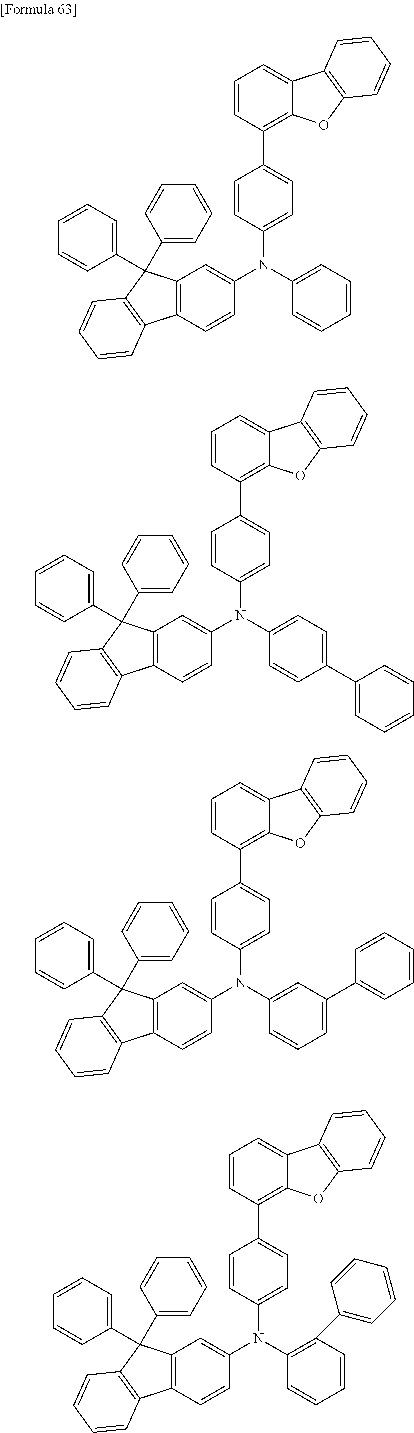

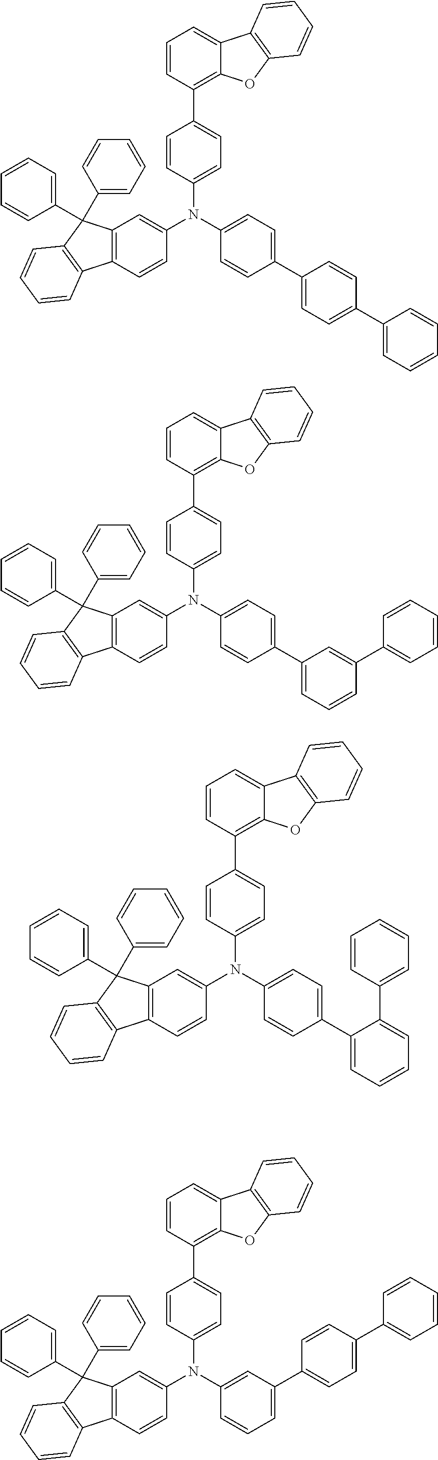

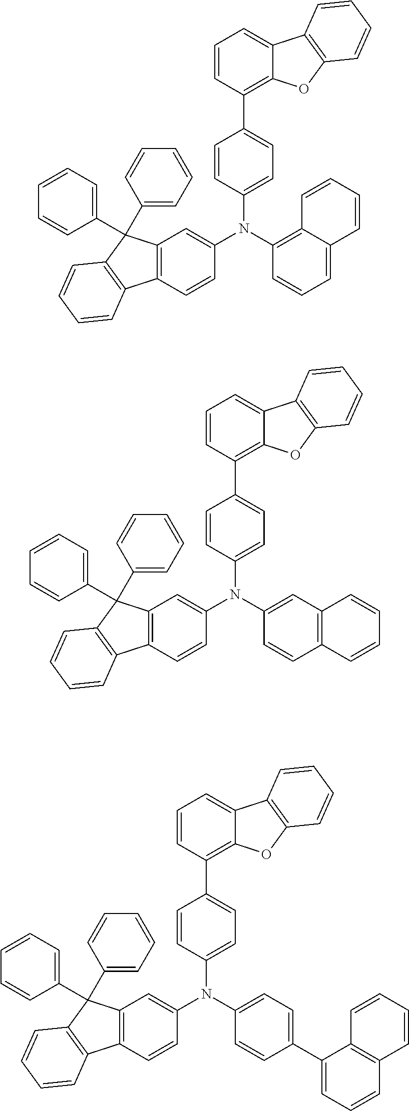 [SMILES: c1ccc(C2(c3ccccc3)c3ccccc3-c3ccc(N(c4ccc(-c5cccc6c5oc5ccccc56)cc4)c4cccc5ccccc45)cc32)cc1 ; c1ccc(C2(c3ccccc3)c3ccccc3-c3ccc(N(c4ccc(-c5cccc6c5oc5ccccc56)cc4)c4ccc5ccccc5c4)cc32)cc1 ; c1ccc(C2(c3ccccc3)c3ccccc3-c3ccc(N(c4ccc(-c5cccc6ccccc56)cc4)c4ccc(-c5cccc6c5oc5ccccc56)cc4)cc32)cc1]

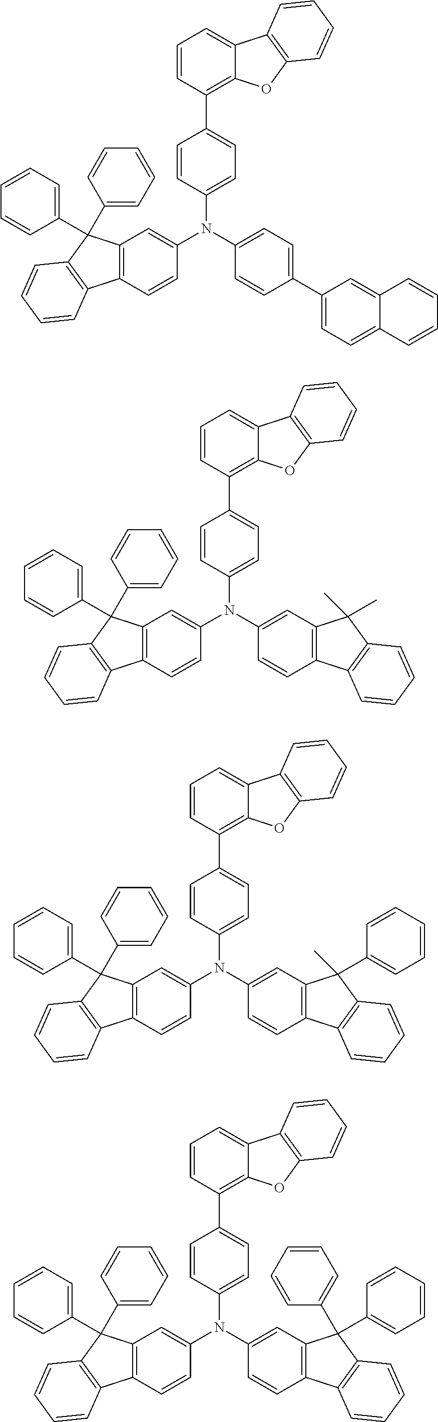

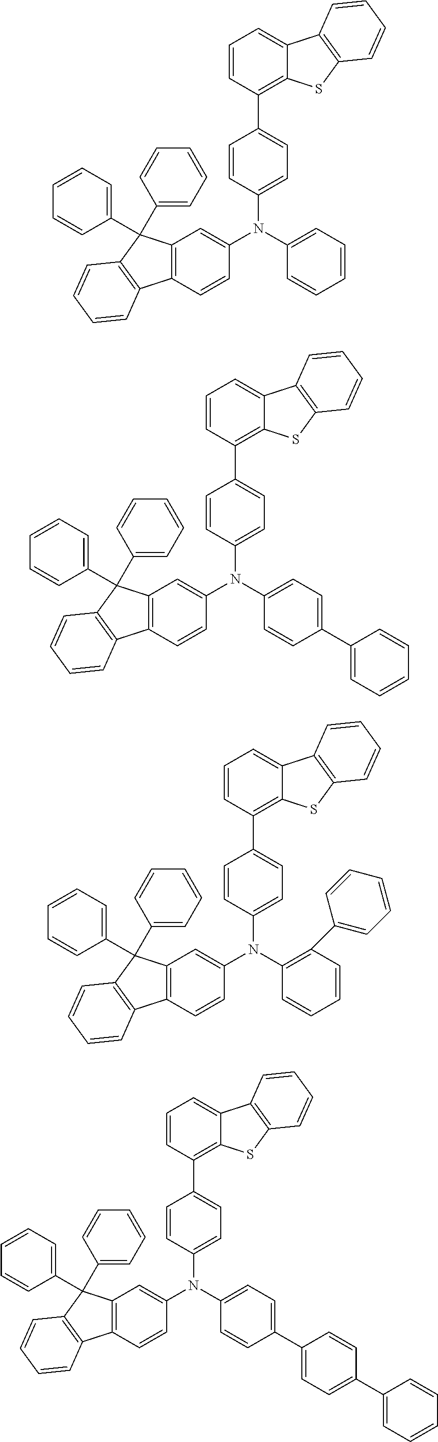

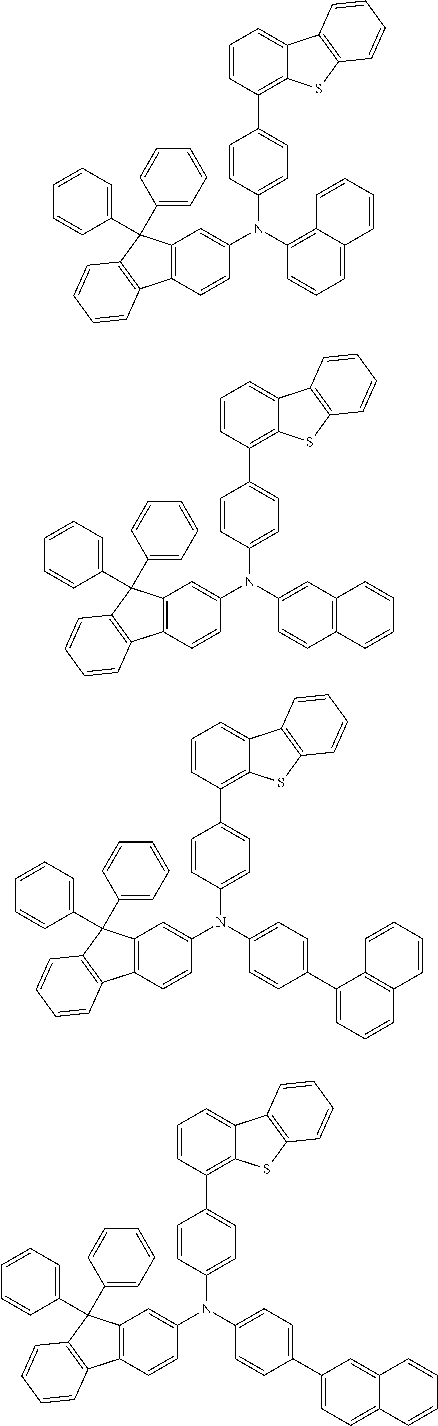 [SMILES: c1ccc(C2(c3ccccc3)c3ccccc3-c3ccc(N(c4ccc(-c5cccc6c5sc5ccccc56)cc4)c4cccc5ccccc45)cc32)cc1 ; c1ccc(C2(c3ccccc3)c3ccccc3-c3ccc(N(c4ccc(-c5cccc6c5sc5ccccc56)cc4)c4ccc5ccccc5c4)cc32)cc1 ; c1ccc(C2(c3ccccc3)c3ccccc3-c3ccc(N(c4ccc(-c5cccc6ccccc56)cc4)c4ccc(-c5cccc6c5sc5ccccc56)cc4)cc32)cc1 ; c1ccc(C2(c3ccccc3)c3ccccc3-c3ccc(N(c4ccc(-c5ccc6ccccc6c5)cc4)c4ccc(-c5cccc6c5sc5ccccc56)cc4)cc32)cc1]

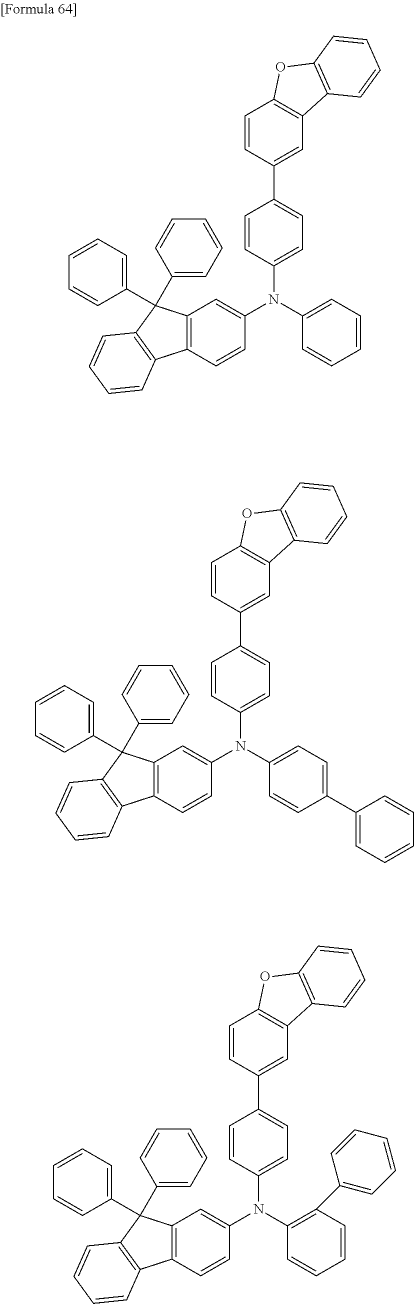 [SMILES: c1ccc(N(c2ccc(-c3ccc4oc5ccccc5c4c3)cc2)c2ccc3c(c2)C(c2ccccc2)(c2ccccc2)c2ccccc2-3)cc1 ; c1ccc(-c2ccc(N(c3ccc(-c4ccc5oc6ccccc6c5c4)cc3)c3ccc4c(c3)C(c3ccccc3)(c3ccccc3)c3ccccc3-4)cc2)cc1 ; c1ccc(-c2ccccc2N(c2ccc(-c3ccc4oc5ccccc5c4c3)cc2)c2ccc3c(c2)C(c2ccccc2)(c2ccccc2)c2ccccc2-3)cc1]

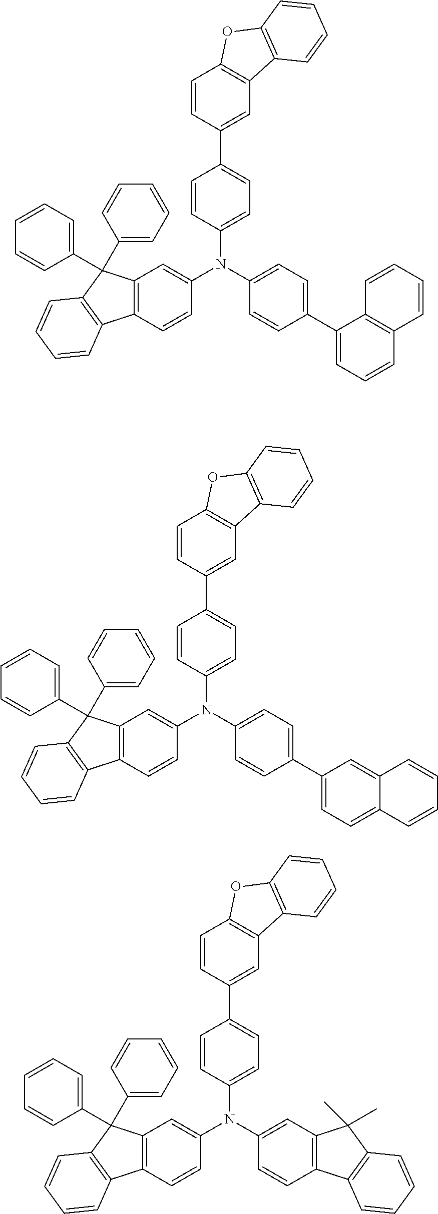

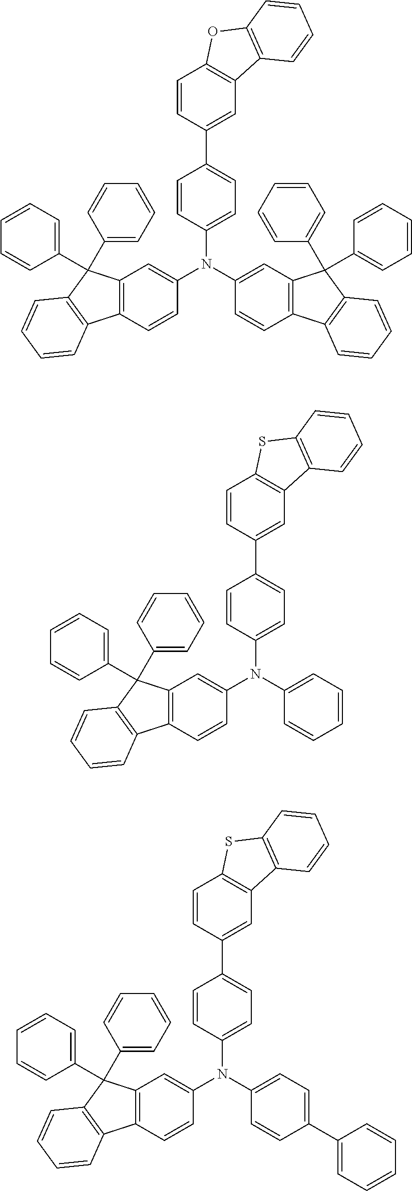 [SMILES: c1ccc(C2(c3ccccc3)c3ccccc3-c3ccc(N(c4ccc(-c5ccc6oc7ccccc7c6c5)cc4)c4ccc5c(c4)C(c4ccccc4)(c4ccccc4)c4ccccc4-5)cc32)cc1 ; c1ccc(N(c2ccc(-c3ccc4sc5ccccc5c4c3)cc2)c2ccc3c(c2)C(c2ccccc2)(c2ccccc2)c2ccccc2-3)cc1 ; c1ccc(-c2ccc(N(c3ccc(-c4ccc5sc6ccccc6c5c4)cc3)c3ccc4c(c3)C(c3ccccc3)(c3ccccc3)c3ccccc3-4)cc2)cc1]

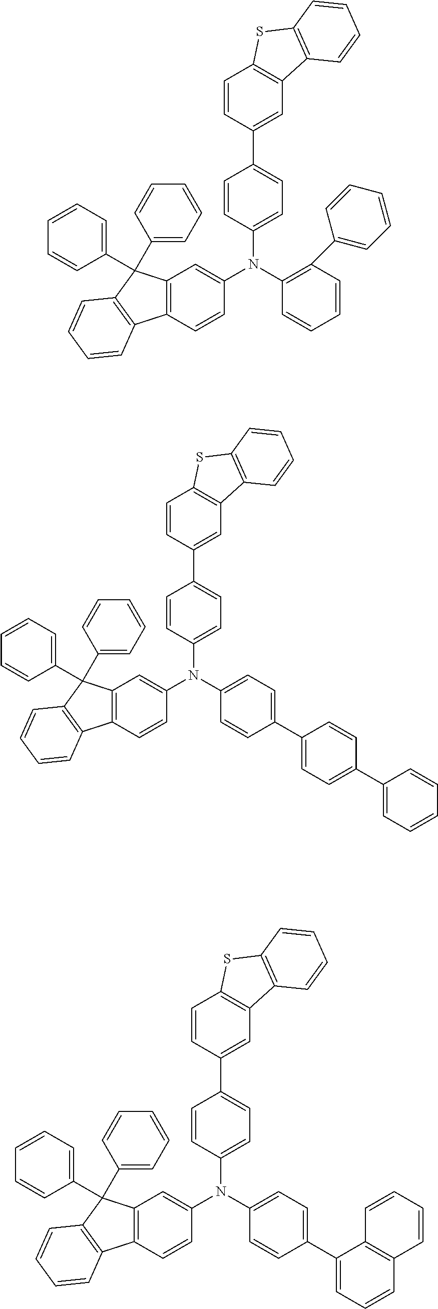 [SMILES: c1ccc(-c2ccccc2N(c2ccc(-c3ccc4sc5ccccc5c4c3)cc2)c2ccc3c(c2)C(c2ccccc2)(c2ccccc2)c2ccccc2-3)cc1 ; c1ccc(-c2ccc(-c3ccc(N(c4ccc(-c5ccc6sc7ccccc7c6c5)cc4)c4ccc5c(c4)C(c4ccccc4)(c4ccccc4)c4ccccc4-5)cc3)cc2)cc1 ; c1ccc(C2(c3ccccc3)c3ccccc3-c3ccc(N(c4ccc(-c5ccc6sc7ccccc7c6c5)cc4)c4ccc(-c5cccc6ccccc56)cc4)cc32)cc1]

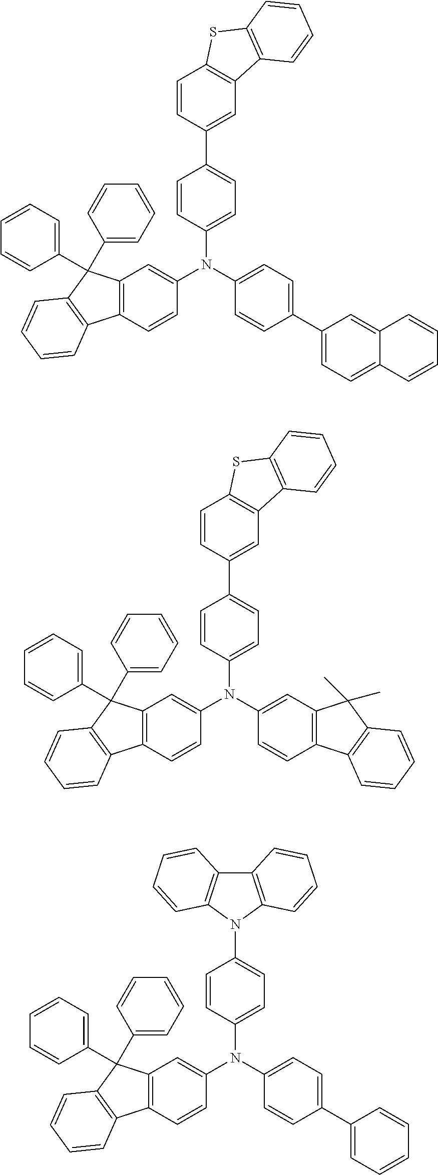 [SMILES: c1ccc(C2(c3ccccc3)c3ccccc3-c3ccc(N(c4ccc(-c5ccc6ccccc6c5)cc4)c4ccc(-c5ccc6sc7ccccc7c6c5)cc4)cc32)cc1 ; CC1(C)c2ccccc2-c2ccc(N(c3ccc(-c4ccc5sc6ccccc6c5c4)cc3)c3ccc4c(c3)C(c3ccccc3)(c3ccccc3)c3ccccc3-4)cc21 ; c1ccc(-c2ccc(N(c3ccc(-n4c5ccccc5c5ccccc54)cc3)c3ccc4c(c3)C(c3ccccc3)(c3ccccc3)c3ccccc3-4)cc2)cc1]

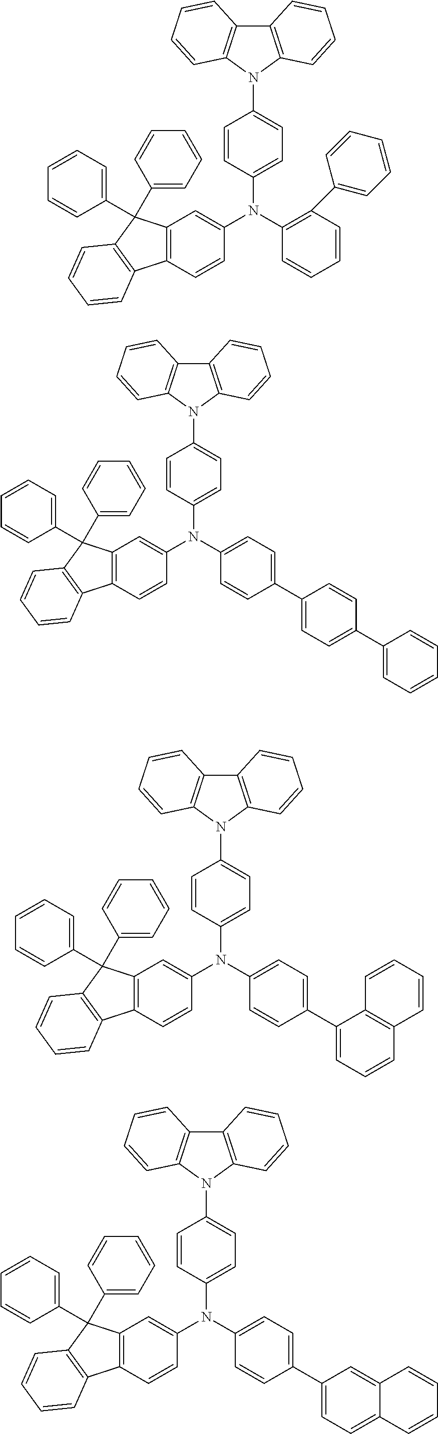

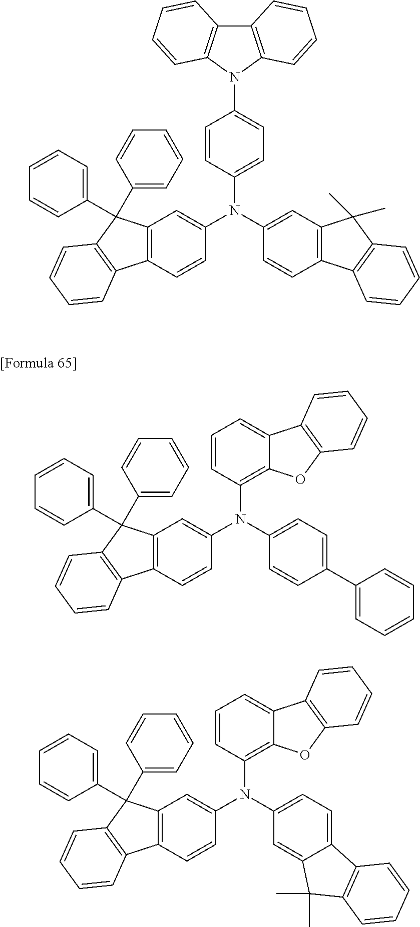

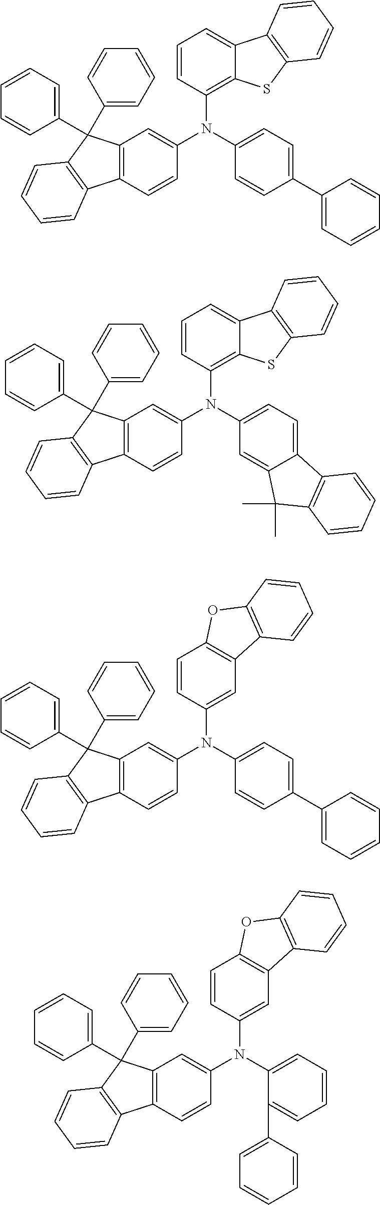

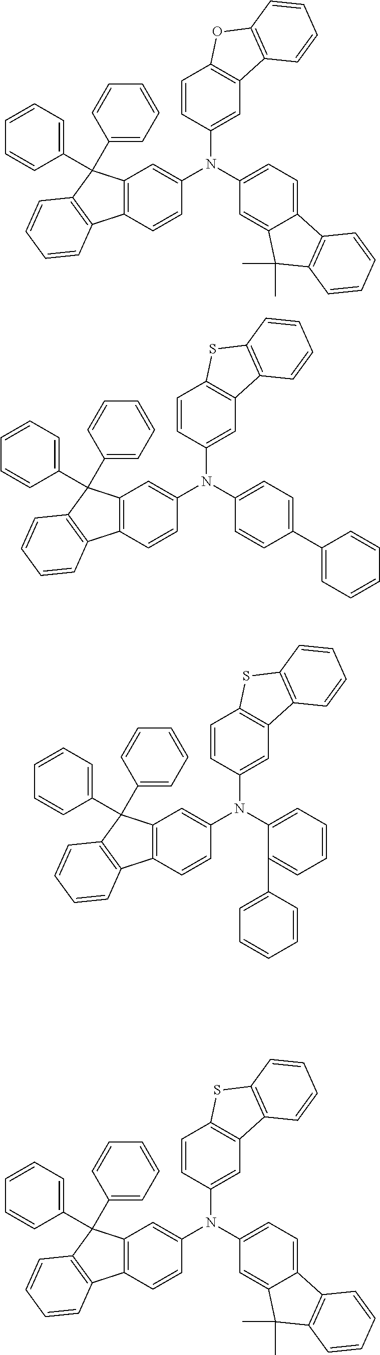

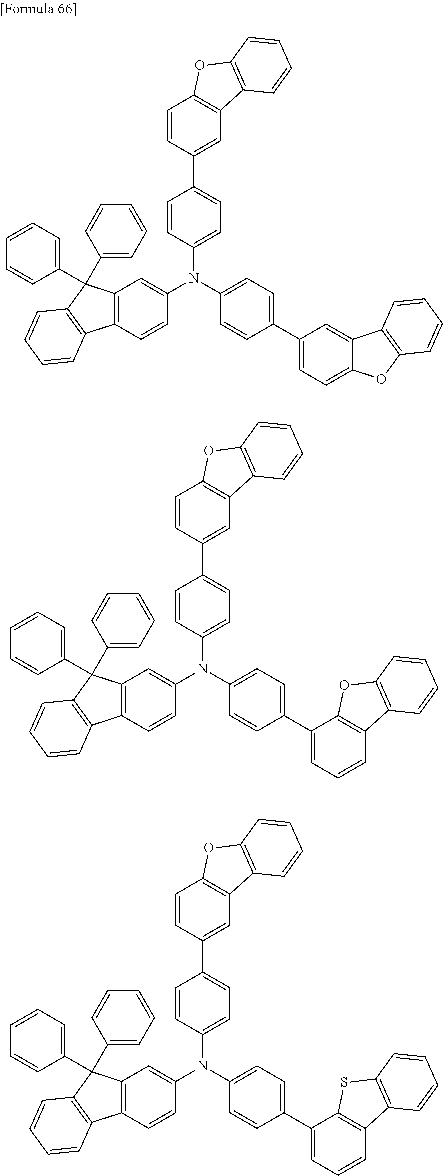 [SMILES: c1ccc(C2(c3ccccc3)c3ccccc3-c3ccc(N(c4ccc(-c5ccc6oc7ccccc7c6c5)cc4)c4ccc(-c5ccc6oc7ccccc7c6c5)cc4)cc32)cc1 ; c1ccc(C2(c3ccccc3)c3ccccc3-c3ccc(N(c4ccc(-c5ccc6oc7ccccc7c6c5)cc4)c4ccc(-c5cccc6c5oc5ccccc56)cc4)cc32)cc1 ; c1ccc(C2(c3ccccc3)c3ccccc3-c3ccc(N(c4ccc(-c5ccc6oc7ccccc7c6c5)cc4)c4ccc(-c5cccc6c5sc5ccccc56)cc4)cc32)cc1]

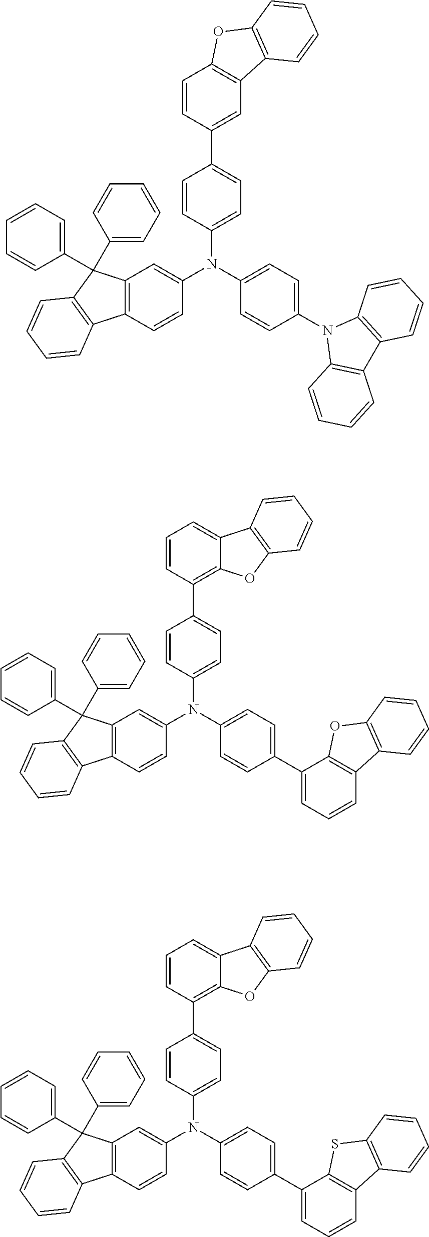 [SMILES: c1ccc(C2(c3ccccc3)c3ccccc3-c3ccc(N(c4ccc(-c5ccc6oc7ccccc7c6c5)cc4)c4ccc(-n5c6ccccc6c6ccccc65)cc4)cc32)cc1 ; c1ccc(C2(c3ccccc3)c3ccccc3-c3ccc(N(c4ccc(-c5cccc6c5oc5ccccc56)cc4)c4ccc(-c5cccc6c5oc5ccccc56)cc4)cc32)cc1 ; c1ccc(C2(c3ccccc3)c3ccccc3-c3ccc(N(c4ccc(-c5cccc6c5oc5ccccc56)cc4)c4ccc(-c5cccc6c5sc5ccccc56)cc4)cc32)cc1]

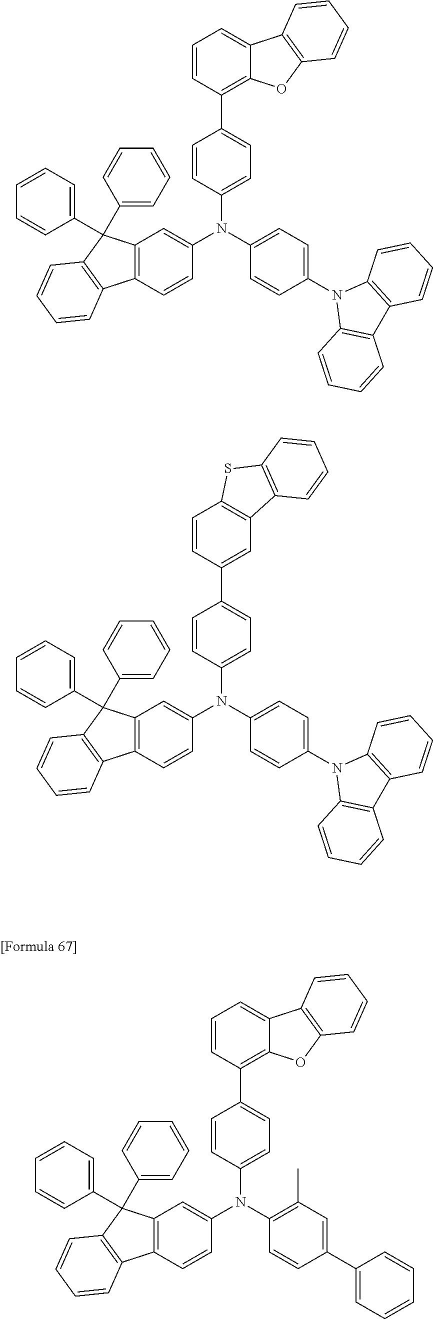 [SMILES: c1ccc(C2(c3ccccc3)c3ccccc3-c3ccc(N(c4ccc(-c5cccc6c5oc5ccccc56)cc4)c4ccc(-n5c6ccccc6c6ccccc65)cc4)cc32)cc1 ; c1ccc(C2(c3ccccc3)c3ccccc3-c3ccc(N(c4ccc(-c5ccc6sc7ccccc7c6c5)cc4)c4ccc(-n5c6ccccc6c6ccccc65)cc4)cc32)cc1 ; Cc1cc(-c2ccccc2)ccc1N(c1ccc(-c2cccc3c2oc2ccccc23)cc1)c1ccc2c(c1)C(c1ccccc1)(c1ccccc1)c1ccccc1-2]

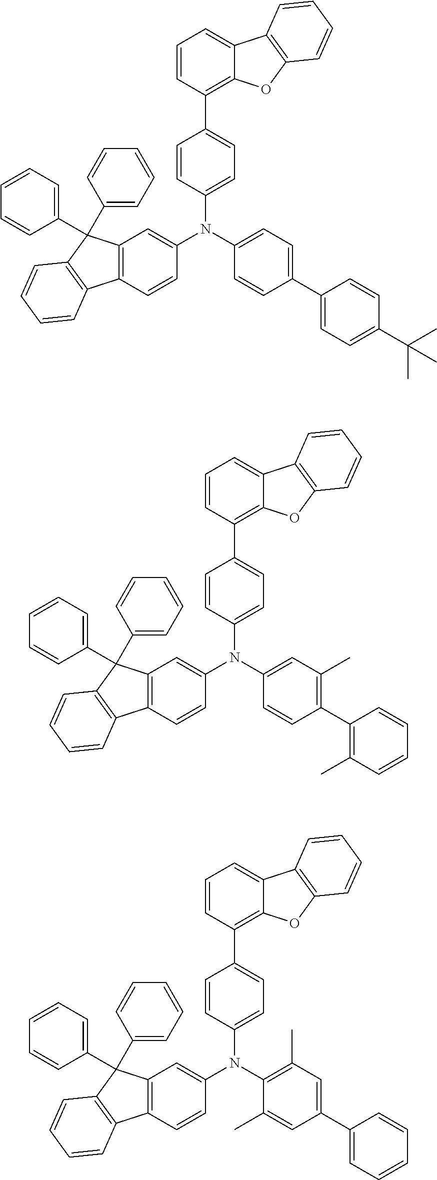 [SMILES: CC(C)(C)c1ccc(-c2ccc(N(c3ccc(-c4cccc5c4oc4ccccc45)cc3)c3ccc4c(c3)C(c3ccccc3)(c3ccccc3)c3ccccc3-4)cc2)cc1 ; Cc1ccccc1-c1ccc(N(c2ccc(-c3cccc4c3oc3ccccc34)cc2)c2ccc3c(c2)C(c2ccccc2)(c2ccccc2)c2ccccc2-3)cc1C ; Cc1cc(-c2ccccc2)cc(C)c1N(c1ccc(-c2cccc3c2oc2ccccc23)cc1)c1ccc2c(c1)C(c1ccccc1)(c1ccccc1)c1ccccc1-2]

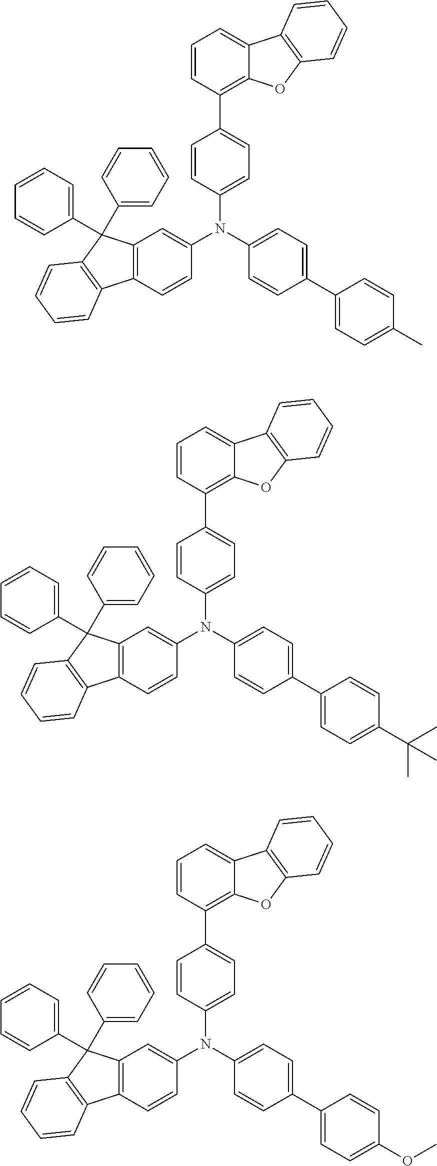 [SMILES: Cc1ccc(-c2ccc(N(c3ccc(-c4cccc5c4oc4ccccc45)cc3)c3ccc4c(c3)C(c3ccccc3)(c3ccccc3)c3ccccc3-4)cc2)cc1 ; CC(C)(C)c1ccc(-c2ccc(N(c3ccc(-c4cccc5c4oc4ccccc45)cc3)c3ccc4c(c3)C(c3ccccc3)(c3ccccc3)c3ccccc3-4)cc2)cc1 ; COc1ccc(-c2ccc(N(c3ccc(-c4cccc5c4oc4ccccc45)cc3)c3ccc4c(c3)C(c3ccccc3)(c3ccccc3)c3ccccc3-4)cc2)cc1]

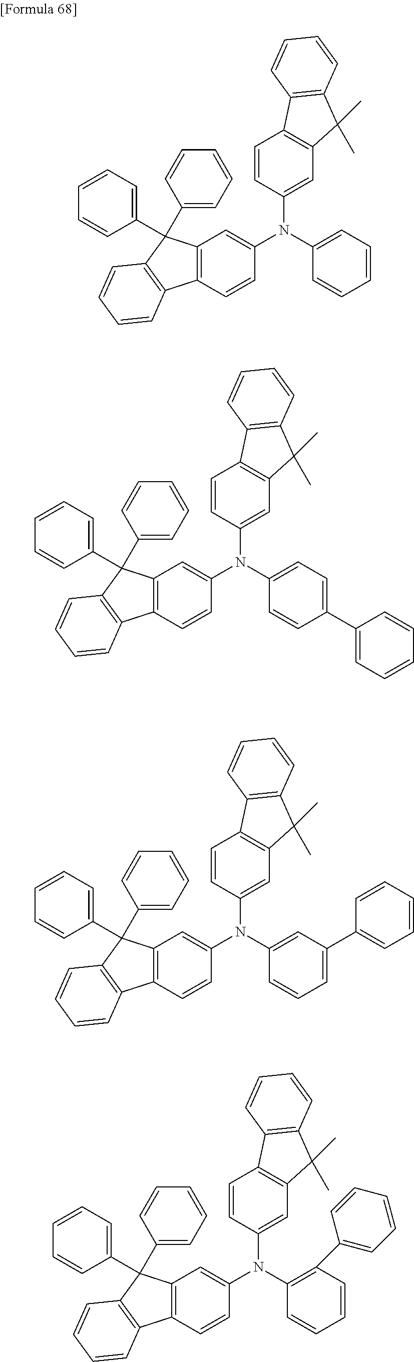 [SMILES: CC1(C)c2ccccc2-c2ccc(N(c3ccccc3)c3ccc4c(c3)C(c3ccccc3)(c3ccccc3)c3ccccc3-4)cc21 ; CC1(C)c2ccccc2-c2ccc(N(c3ccc(-c4ccccc4)cc3)c3ccc4c(c3)C(c3ccccc3)(c3ccccc3)c3ccccc3-4)cc21 ; CC1(C)c2ccccc2-c2ccc(N(c3cccc(-c4ccccc4)c3)c3ccc4c(c3)C(c3ccccc3)(c3ccccc3)c3ccccc3-4)cc21 ; CC1(C)c2ccccc2-c2ccc(N(c3ccc4c(c3)C(c3ccccc3)(c3ccccc3)c3ccccc3-4)c3ccccc3-c3ccccc3)cc21]

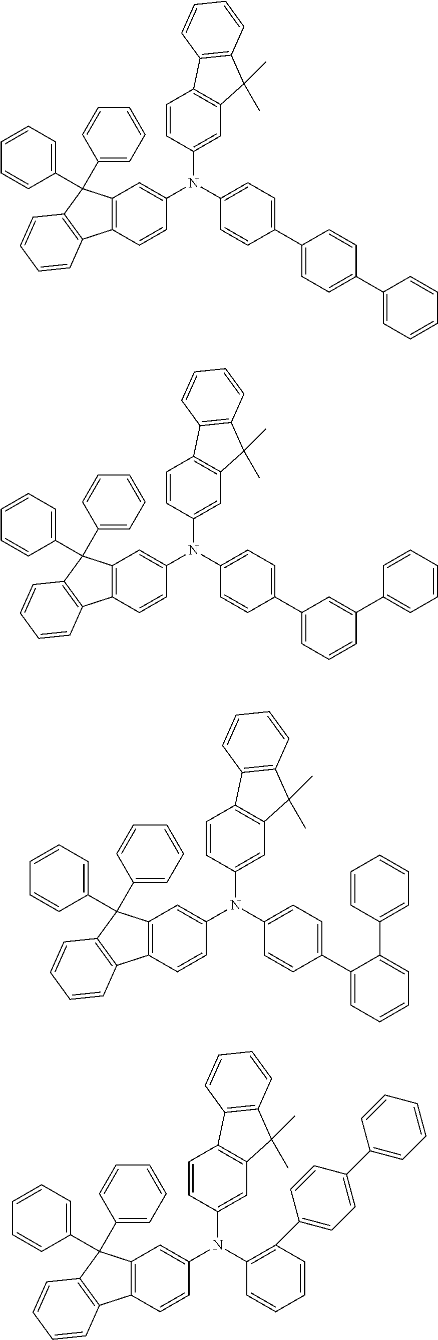 [SMILES: CC1(C)c2ccccc2-c2ccc(N(c3ccc(-c4ccc(-c5ccccc5)cc4)cc3)c3ccc4c(c3)C(c3ccccc3)(c3ccccc3)c3ccccc3-4)cc21 ; CC1(C)c2ccccc2-c2ccc(N(c3ccc(-c4cccc(-c5ccccc5)c4)cc3)c3ccc4c(c3)C(c3ccccc3)(c3ccccc3)c3ccccc3-4)cc21 ; CC1(C)c2ccccc2-c2ccc(N(c3ccc(-c4ccccc4-c4ccccc4)cc3)c3ccc4c(c3)C(c3ccccc3)(c3ccccc3)c3ccccc3-4)cc21 ; CC1(C)c2ccccc2-c2ccc(N(c3ccc4c(c3)C(c3ccccc3)(c3ccccc3)c3ccccc3-4)c3ccccc3-c3ccc(-c4ccccc4)cc3)cc21]

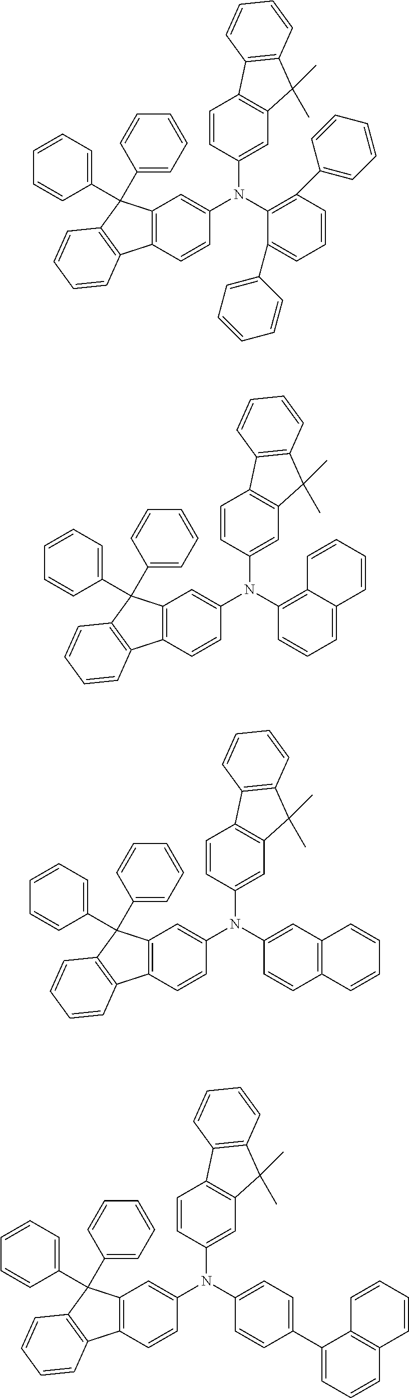 [SMILES: CC1(C)c2ccccc2-c2ccc(N(c3ccc4c(c3)C(c3ccccc3)(c3ccccc3)c3ccccc3-4)c3c(-c4ccccc4)cccc3-c3ccccc3)cc21 ; CC1(C)c2ccccc2-c2ccc(N(c3ccc4c(c3)C(c3ccccc3)(c3ccccc3)c3ccccc3-4)c3cccc4ccccc34)cc21 ; CC1(C)c2ccccc2-c2ccc(N(c3ccc4c(c3)C(c3ccccc3)(c3ccccc3)c3ccccc3-4)c3ccc4ccccc4c3)cc21 ; CC1(C)c2ccccc2-c2ccc(N(c3ccc(-c4cccc5ccccc45)cc3)c3ccc4c(c3)C(c3ccccc3)(c3ccccc3)c3ccccc3-4)cc21]

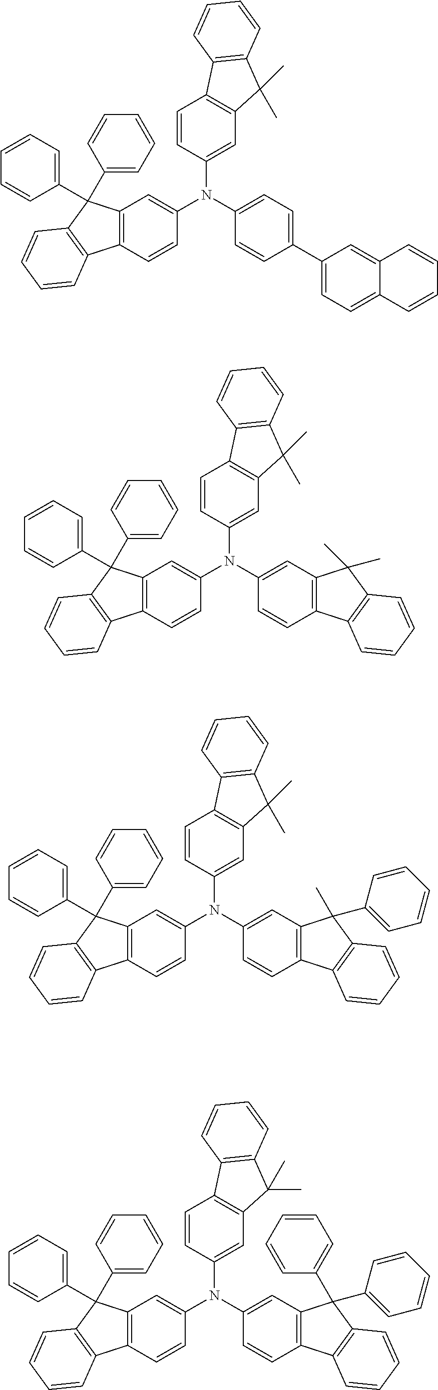 [SMILES: CC1(C)c2ccccc2-c2ccc(N(c3ccc(-c4ccc5ccccc5c4)cc3)c3ccc4c(c3)C(c3ccccc3)(c3ccccc3)c3ccccc3-4)cc21 ; CC1(C)c2ccccc2-c2ccc(N(c3ccc4c(c3)C(C)(C)c3ccccc3-4)c3ccc4c(c3)C(c3ccccc3)(c3ccccc3)c3ccccc3-4)cc21 ; CC1(C)c2ccccc2-c2ccc(N(c3ccc4c(c3)C(C)(c3ccccc3)c3ccccc3-4)c3ccc4c(c3)C(c3ccccc3)(c3ccccc3)c3ccccc3-4)cc21 ; CC1(C)c2ccccc2-c2ccc(N(c3ccc4c(c3)C(c3ccccc3)(c3ccccc3)c3ccccc3-4)c3ccc4c(c3)C(c3ccccc3)(c3ccccc3)c3ccccc3-4)cc21]

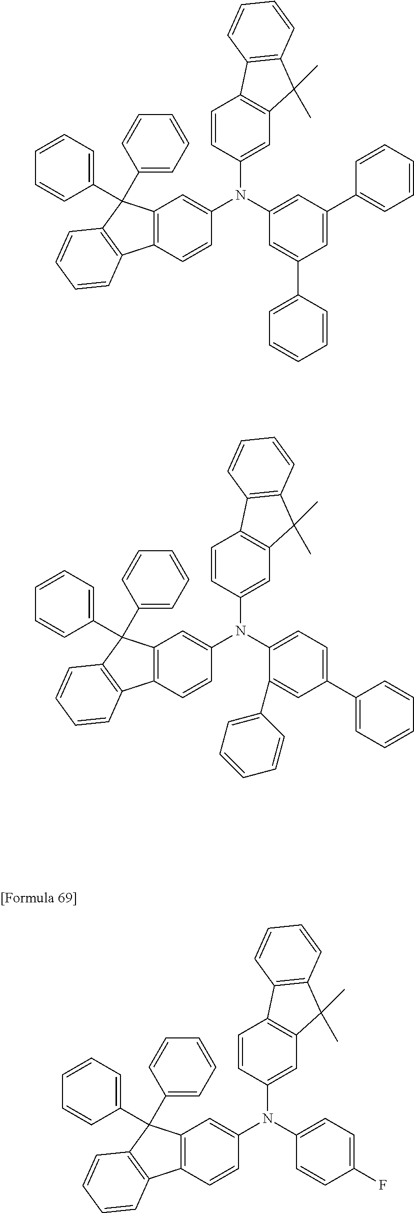 [SMILES: CC1(C)c2ccccc2-c2ccc(N(c3cc(-c4ccccc4)cc(-c4ccccc4)c3)c3ccc4c(c3)C(c3ccccc3)(c3ccccc3)c3ccccc3-4)cc21 ; CC1(C)c2ccccc2-c2ccc(N(c3ccc4c(c3)C(c3ccccc3)(c3ccccc3)c3ccccc3-4)c3ccc(-c4ccccc4)cc3-c3ccccc3)cc21 ; CC1(C)c2ccccc2-c2ccc(N(c3ccc(F)cc3)c3ccc4c(c3)C(c3ccccc3)(c3ccccc3)c3ccccc3-4)cc21]

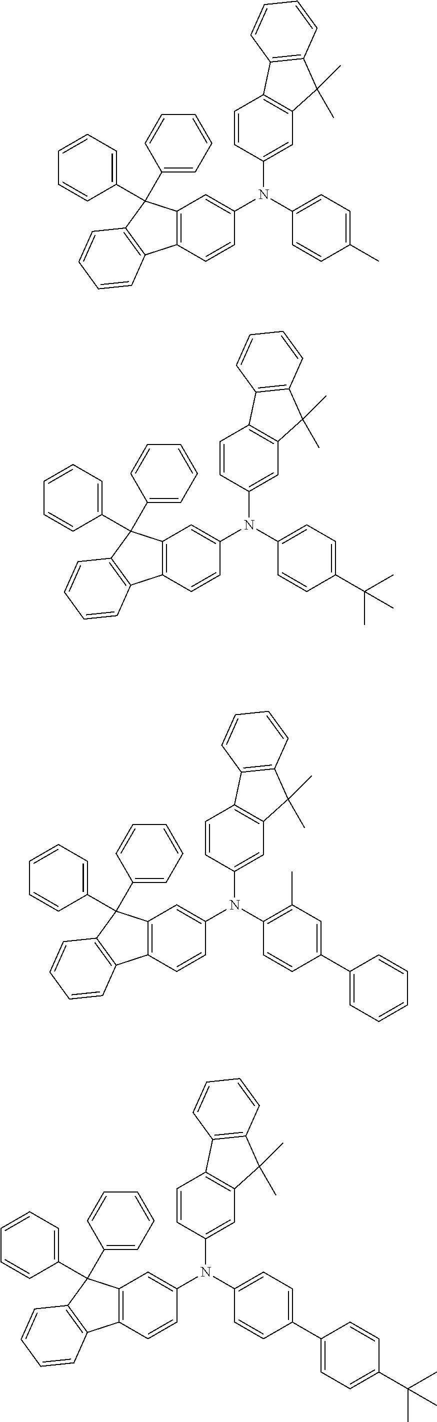 [SMILES: Cc1ccc(N(c2ccc3c(c2)C(C)(C)c2ccccc2-3)c2ccc3c(c2)C(c2ccccc2)(c2ccccc2)c2ccccc2-3)cc1 ; CC(C)(C)c1ccc(N(c2ccc3c(c2)C(C)(C)c2ccccc2-3)c2ccc3c(c2)C(c2ccccc2)(c2ccccc2)c2ccccc2-3)cc1 ; Cc1cc(-c2ccccc2)ccc1N(c1ccc2c(c1)C(C)(C)c1ccccc1-2)c1ccc2c(c1)C(c1ccccc1)(c1ccccc1)c1ccccc1-2 ; CC(C)(C)c1ccc(-c2ccc(N(c3ccc4c(c3)C(C)(C)c3ccccc3-4)c3ccc4c(c3)C(c3ccccc3)(c3ccccc3)c3ccccc3-4)cc2)cc1]

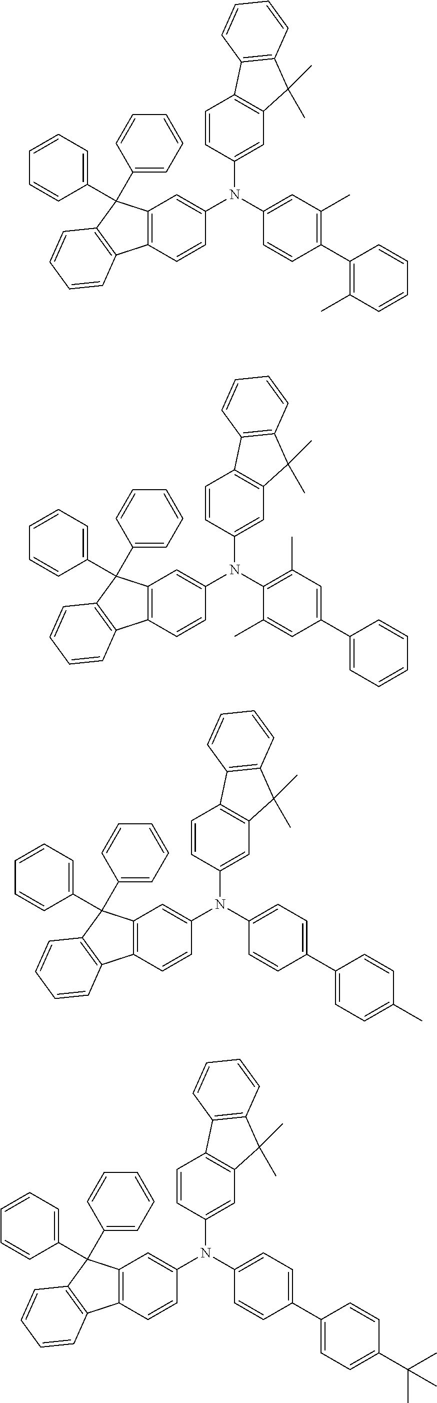 [SMILES: Cc1ccccc1-c1ccc(N(c2ccc3c(c2)C(C)(C)c2ccccc2-3)c2ccc3c(c2)C(c2ccccc2)(c2ccccc2)c2ccccc2-3)cc1C ; Cc1cc(-c2ccccc2)cc(C)c1N(c1ccc2c(c1)C(C)(C)c1ccccc1-2)c1ccc2c(c1)C(c1ccccc1)(c1ccccc1)c1ccccc1-2 ; Cc1ccc(-c2ccc(N(c3ccc4c(c3)C(C)(C)c3ccccc3-4)c3ccc4c(c3)C(c3ccccc3)(c3ccccc3)c3ccccc3-4)cc2)cc1 ; CC(C)(C)c1ccc(-c2ccc(N(c3ccc4c(c3)C(C)(C)c3ccccc3-4)c3ccc4c(c3)C(c3ccccc3)(c3ccccc3)c3ccccc3-4)cc2)cc1]

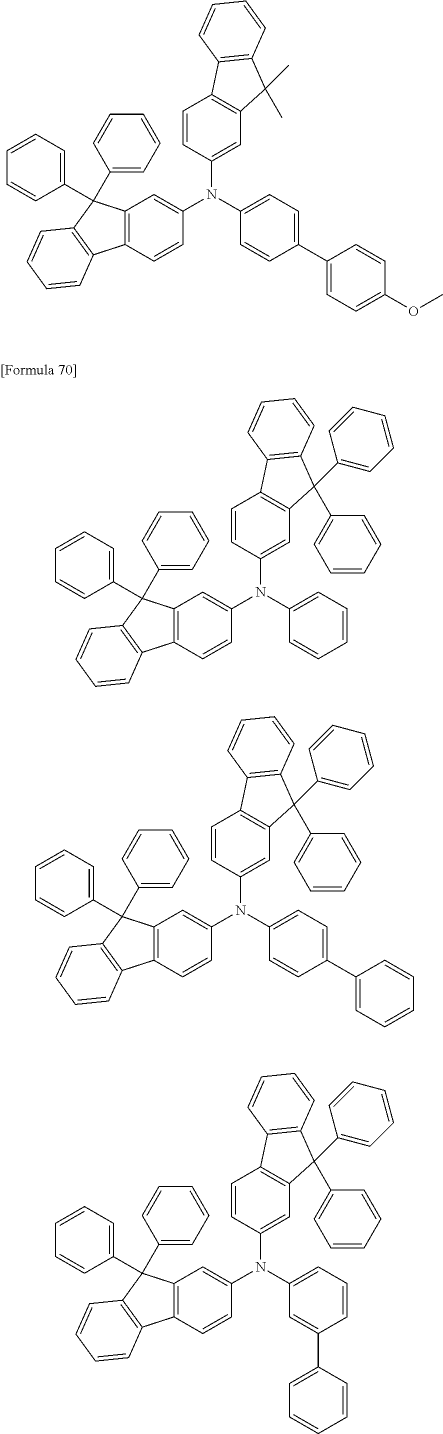 [SMILES: COc1ccc(-c2ccc(N(c3ccc4c(c3)C(C)(C)c3ccccc3-4)c3ccc4c(c3)C(c3ccccc3)(c3ccccc3)c3ccccc3-4)cc2)cc1 ; c1ccc(N(c2ccc3c(c2)C(c2ccccc2)(c2ccccc2)c2ccccc2-3)c2ccc3c(c2)C(c2ccccc2)(c2ccccc2)c2ccccc2-3)cc1 ; c1ccc(-c2ccc(N(c3ccc4c(c3)C(c3ccccc3)(c3ccccc3)c3ccccc3-4)c3ccc4c(c3)C(c3ccccc3)(c3ccccc3)c3ccccc3-4)cc2)cc1 ; c1ccc(-c2cccc(N(c3ccc4c(c3)C(c3ccccc3)(c3ccccc3)c3ccccc3-4)c3ccc4c(c3)C(c3ccccc3)(c3ccccc3)c3ccccc3-4)c2)cc1]

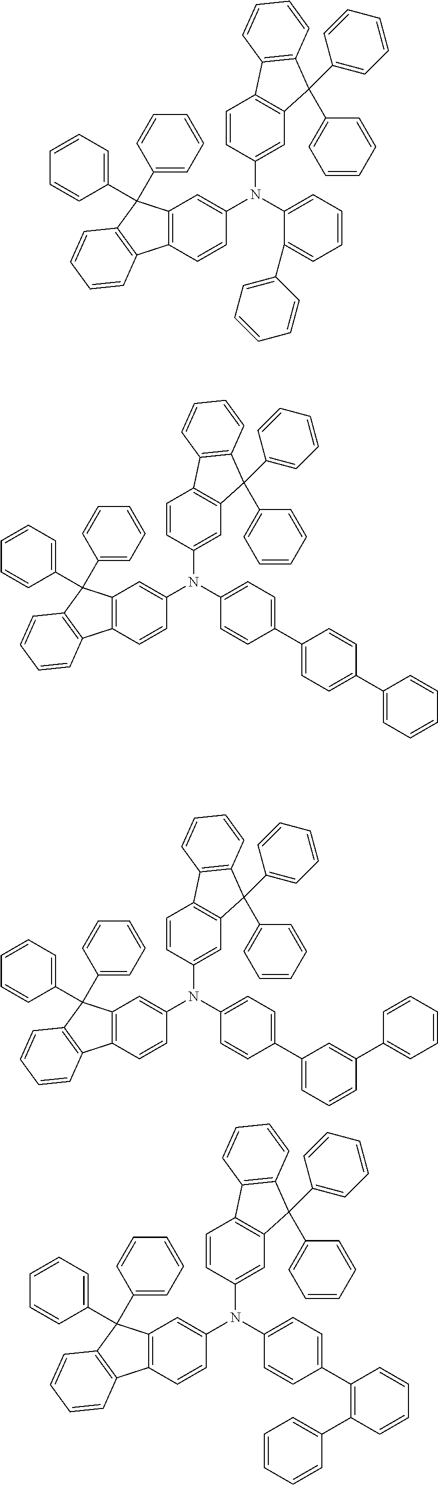

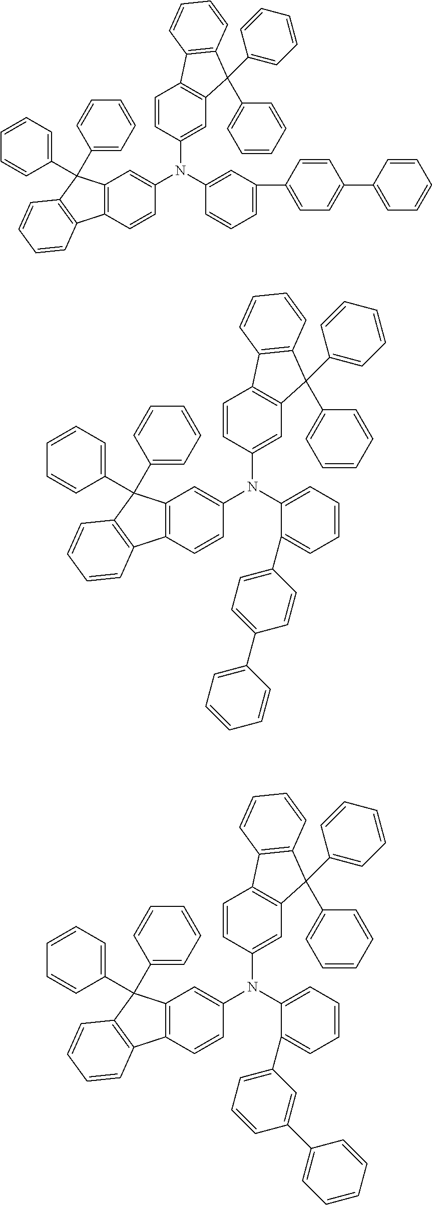 [SMILES: c1ccc(-c2ccc(-c3cccc(N(c4ccc5c(c4)C(c4ccccc4)(c4ccccc4)c4ccccc4-5)c4ccc5c(c4)C(c4ccccc4)(c4ccccc4)c4ccccc4-5)c3)cc2)cc1 ; c1ccc(-c2ccc(-c3ccccc3N(c3ccc4c(c3)C(c3ccccc3)(c3ccccc3)c3ccccc3-4)c3ccc4c(c3)C(c3ccccc3)(c3ccccc3)c3ccccc3-4)cc2)cc1 ; c1ccc(-c2cccc(-c3ccccc3N(c3ccc4c(c3)C(c3ccccc3)(c3ccccc3)c3ccccc3-4)c3ccc4c(c3)C(c3ccccc3)(c3ccccc3)c3ccccc3-4)c2)cc1]

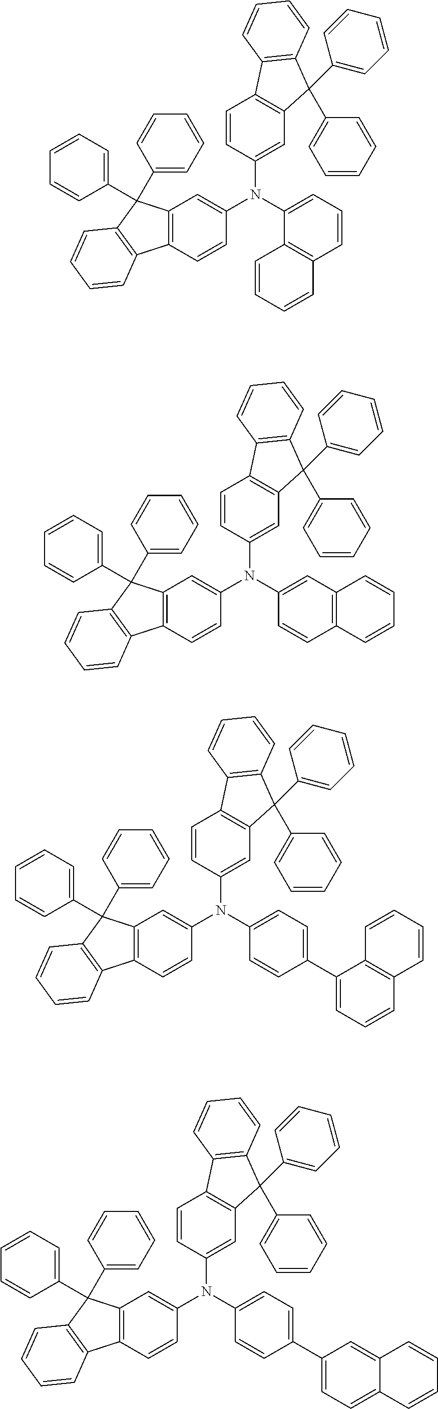 [SMILES: c1ccc(C2(c3ccccc3)c3ccccc3-c3ccc(N(c4ccc5c(c4)C(c4ccccc4)(c4ccccc4)c4ccccc4-5)c4cccc5ccccc45)cc32)cc1 ; c1ccc(C2(c3ccccc3)c3ccccc3-c3ccc(N(c4ccc5c(c4)C(c4ccccc4)(c4ccccc4)c4ccccc4-5)c4ccc5ccccc5c4)cc32)cc1 ; c1ccc(C2(c3ccccc3)c3ccccc3-c3ccc(N(c4ccc(-c5cccc6ccccc56)cc4)c4ccc5c(c4)C(c4ccccc4)(c4ccccc4)c4ccccc4-5)cc32)cc1 ; c1ccc(C2(c3ccccc3)c3ccccc3-c3ccc(N(c4ccc(-c5ccc6ccccc6c5)cc4)c4ccc5c(c4)C(c4ccccc4)(c4ccccc4)c4ccccc4-5)cc32)cc1]

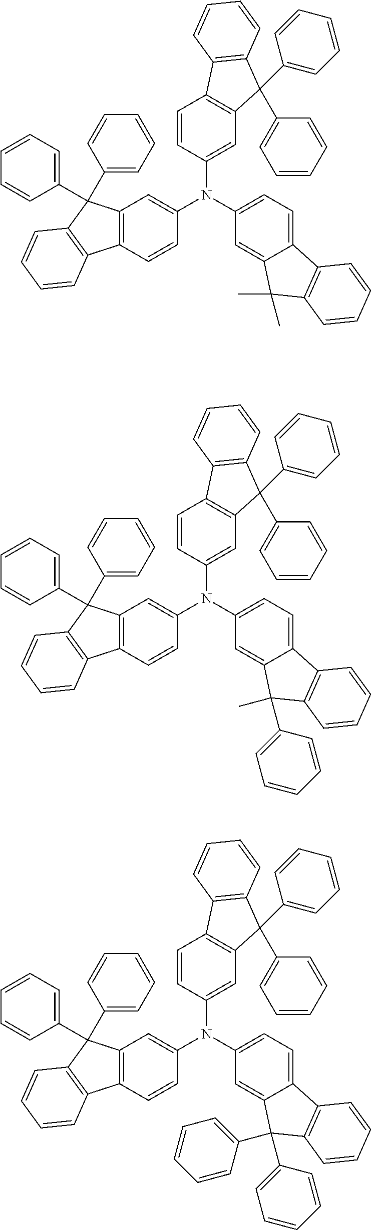 [SMILES: CC1(C)c2ccccc2-c2ccc(N(c3ccc4c(c3)C(c3ccccc3)(c3ccccc3)c3ccccc3-4)c3ccc4c(c3)C(c3ccccc3)(c3ccccc3)c3ccccc3-4)cc21 ; CC1(c2ccccc2)c2ccccc2-c2ccc(N(c3ccc4c(c3)C(c3ccccc3)(c3ccccc3)c3ccccc3-4)c3ccc4c(c3)C(c3ccccc3)(c3ccccc3)c3ccccc3-4)cc21 ; c1ccc(C2(c3ccccc3)c3ccccc3-c3ccc(N(c4ccc5c(c4)C(c4ccccc4)(c4ccccc4)c4ccccc4-5)c4ccc5c(c4)C(c4ccccc4)(c4ccccc4)c4ccccc4-5)cc32)cc1]

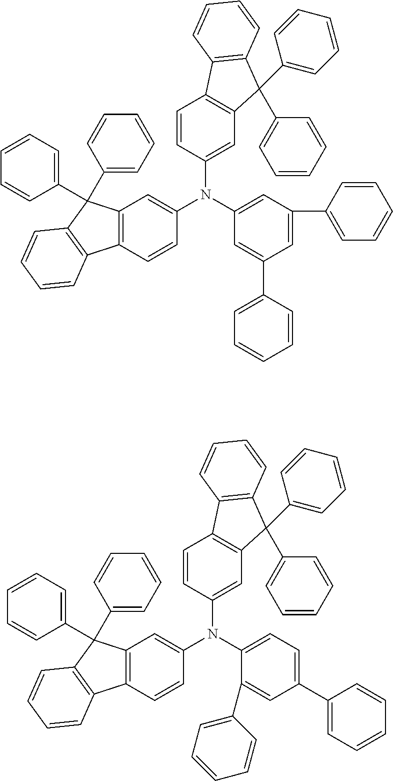 [SMILES: c1ccc(-c2cc(-c3ccccc3)cc(N(c3ccc4c(c3)C(c3ccccc3)(c3ccccc3)c3ccccc3-4)c3ccc4c(c3)C(c3ccccc3)(c3ccccc3)c3ccccc3-4)c2)cc1 ; c1ccc(-c2ccc(N(c3ccc4c(c3)C(c3ccccc3)(c3ccccc3)c3ccccc3-4)c3ccc4c(c3)C(c3ccccc3)(c3ccccc3)c3ccccc3-4)c(-c3ccccc3)c2)cc1]

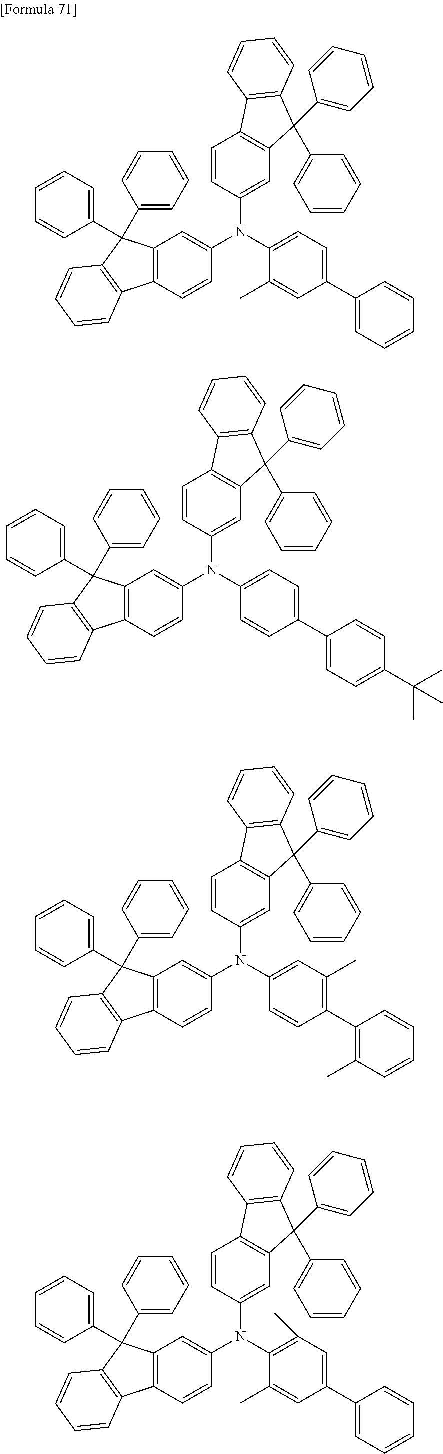 [SMILES: Cc1cc(-c2ccccc2)ccc1N(c1ccc2c(c1)C(c1ccccc1)(c1ccccc1)c1ccccc1-2)c1ccc2c(c1)C(c1ccccc1)(c1ccccc1)c1ccccc1-2 ; CC(C)(C)c1ccc(-c2ccc(N(c3ccc4c(c3)C(c3ccccc3)(c3ccccc3)c3ccccc3-4)c3ccc4c(c3)C(c3ccccc3)(c3ccccc3)c3ccccc3-4)cc2)cc1 ; Cc1ccccc1-c1ccc(N(c2ccc3c(c2)C(c2ccccc2)(c2ccccc2)c2ccccc2-3)c2ccc3c(c2)C(c2ccccc2)(c2ccccc2)c2ccccc2-3)cc1C ; Cc1cc(-c2ccccc2)cc(C)c1N(c1ccc2c(c1)C(c1ccccc1)(c1ccccc1)c1ccccc1-2)c1ccc2c(c1)C(c1ccccc1)(c1ccccc1)c1ccccc1-2]

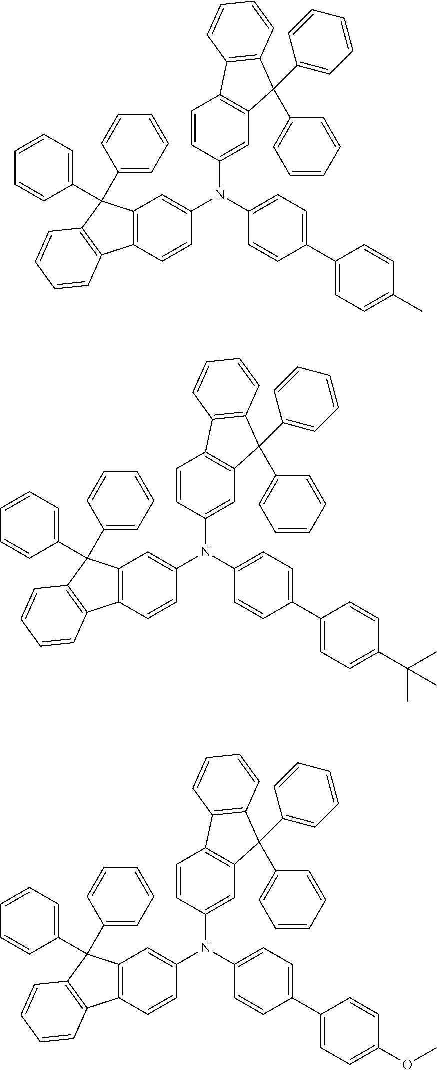 [SMILES: Cc1ccc(-c2ccc(N(c3ccc4c(c3)C(c3ccccc3)(c3ccccc3)c3ccccc3-4)c3ccc4c(c3)C(c3ccccc3)(c3ccccc3)c3ccccc3-4)cc2)cc1 ; CC(C)(C)c1ccc(-c2ccc(N(c3ccc4c(c3)C(c3ccccc3)(c3ccccc3)c3ccccc3-4)c3ccc4c(c3)C(c3ccccc3)(c3ccccc3)c3ccccc3-4)cc2)cc1 ; COc1ccc(-c2ccc(N(c3ccc4c(c3)C(c3ccccc3)(c3ccccc3)c3ccccc3-4)c3ccc4c(c3)C(c3ccccc3)(c3ccccc3)c3ccccc3-4)cc2)cc1]

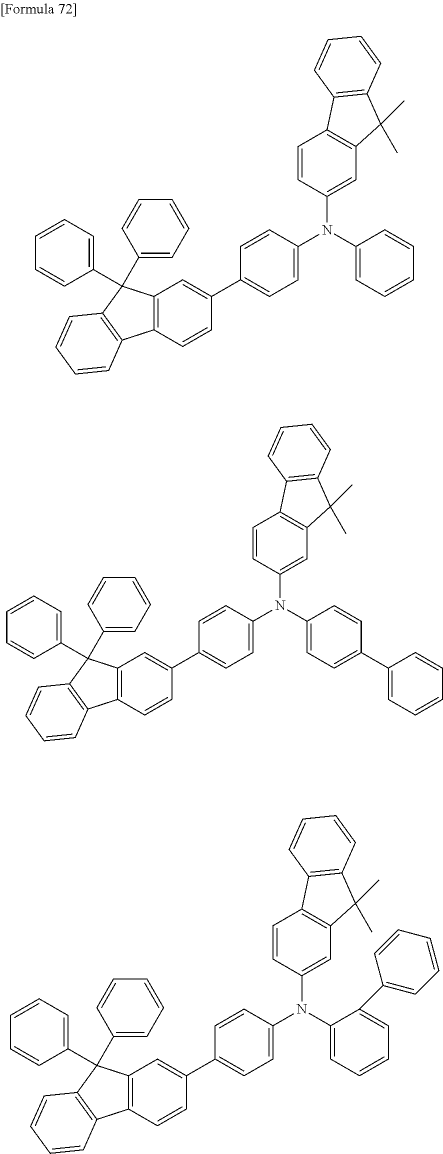 [SMILES: CC1(C)c2ccccc2-c2ccc(N(c3ccccc3)c3ccc(-c4ccc5c(c4)C(c4ccccc4)(c4ccccc4)c4ccccc4-5)cc3)cc21 ; CC1(C)c2ccccc2-c2ccc(N(c3ccc(-c4ccccc4)cc3)c3ccc(-c4ccc5c(c4)C(c4ccccc4)(c4ccccc4)c4ccccc4-5)cc3)cc21 ; CC1(C)c2ccccc2-c2ccc(N(c3ccc(-c4ccc5c(c4)C(c4ccccc4)(c4ccccc4)c4ccccc4-5)cc3)c3ccccc3-c3ccccc3)cc21]

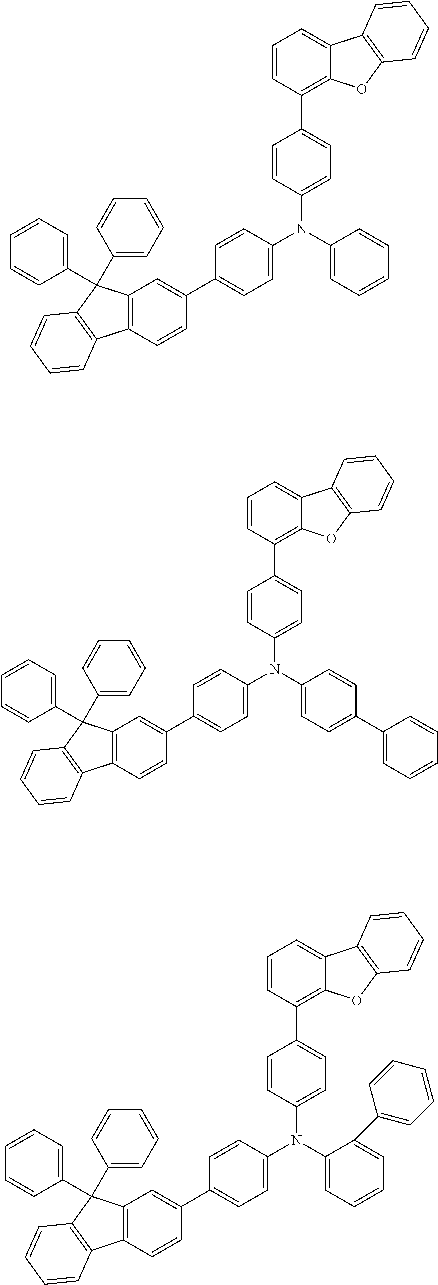 [SMILES: c1ccc(N(c2ccc(-c3ccc4c(c3)C(c3ccccc3)(c3ccccc3)c3ccccc3-4)cc2)c2ccc(-c3cccc4c3oc3ccccc34)cc2)cc1 ; c1ccc(-c2ccc(N(c3ccc(-c4ccc5c(c4)C(c4ccccc4)(c4ccccc4)c4ccccc4-5)cc3)c3ccc(-c4cccc5c4oc4ccccc45)cc3)cc2)cc1 ; c1ccc(-c2ccccc2N(c2ccc(-c3ccc4c(c3)C(c3ccccc3)(c3ccccc3)c3ccccc3-4)cc2)c2ccc(-c3cccc4c3oc3ccccc34)cc2)cc1]

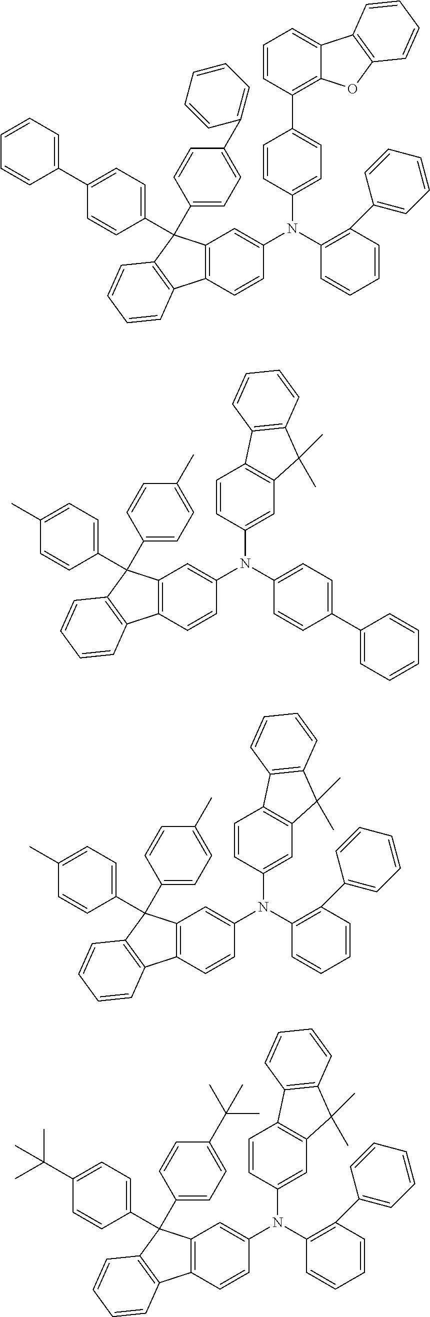 [SMILES: c1ccc(-c2ccc(C3(c4ccc(-c5ccccc5)cc4)c4ccccc4-c4ccc(N(c5ccc(-c6cccc7c6oc6ccccc67)cc5)c5ccccc5-c5ccccc5)cc43)cc2)cc1 ; Cc1ccc(C2(c3ccc(C)cc3)c3ccccc3-c3ccc(N(c4ccc(-c5ccccc5)cc4)c4ccc5c(c4)C(C)(C)c4ccccc4-5)cc32)cc1 ; Cc1ccc(C2(c3ccc(C)cc3)c3ccccc3-c3ccc(N(c4ccc5c(c4)C(C)(C)c4ccccc4-5)c4ccccc4-c4ccccc4)cc32)cc1 ; CC(C)(C)c1ccc(C2(c3ccc(C(C)(C)C)cc3)c3ccccc3-c3ccc(N(c4ccc5c(c4)C(C)(C)c4ccccc4-5)c4ccccc4-c4ccccc4)cc32)cc1]

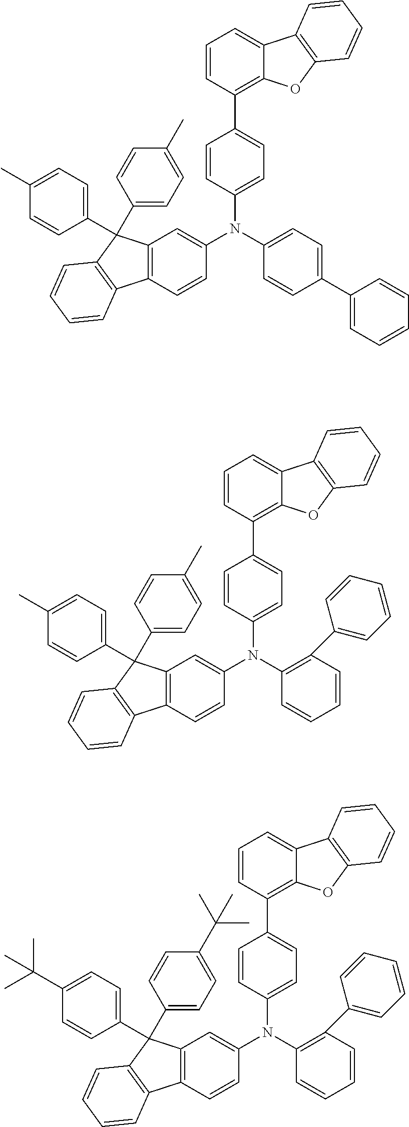 [SMILES: Cc1ccc(C2(c3ccc(C)cc3)c3ccccc3-c3ccc(N(c4ccc(-c5ccccc5)cc4)c4ccc(-c5cccc6c5oc5ccccc56)cc4)cc32)cc1 ; Cc1ccc(C2(c3ccc(C)cc3)c3ccccc3-c3ccc(N(c4ccc(-c5cccc6c5oc5ccccc56)cc4)c4ccccc4-c4ccccc4)cc32)cc1 ; CC(C)(C)c1ccc(C2(c3ccc(C(C)(C)C)cc3)c3ccccc3-c3ccc(N(c4ccc(-c5cccc6c5oc5ccccc56)cc4)c4ccccc4-c4ccccc4)cc32)cc1]

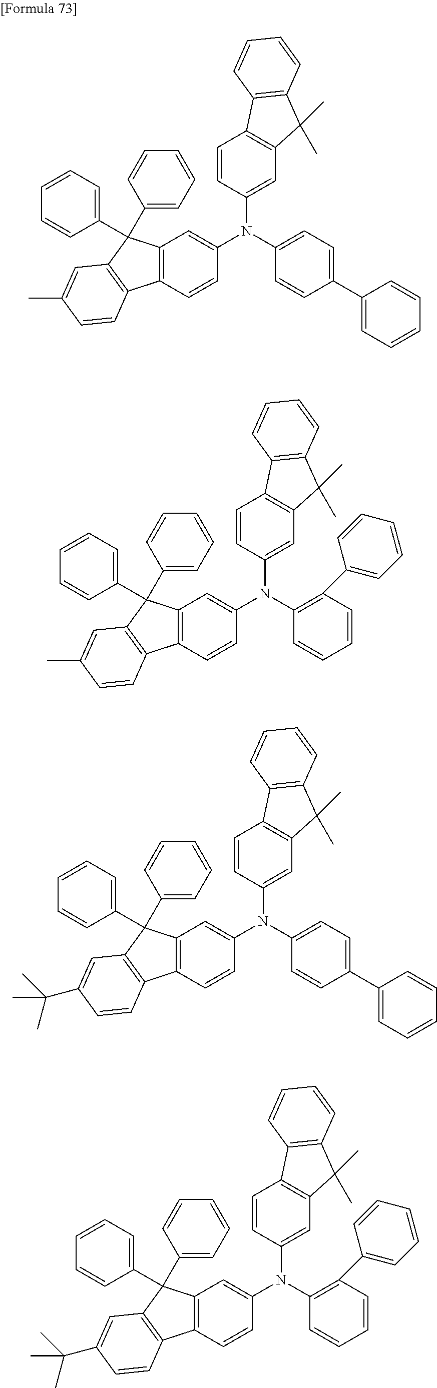 [SMILES: Cc1ccc2c(c1)C(c1ccccc1)(c1ccccc1)c1cc(N(c3ccc(-c4ccccc4)cc3)c3ccc4c(c3)C(C)(C)c3ccccc3-4)ccc1-2 ; Cc1ccc2c(c1)C(c1ccccc1)(c1ccccc1)c1cc(N(c3ccc4c(c3)C(C)(C)c3ccccc3-4)c3ccccc3-c3ccccc3)ccc1-2 ; CC(C)(C)c1ccc2c(c1)C(c1ccccc1)(c1ccccc1)c1cc(N(c3ccc(-c4ccccc4)cc3)c3ccc4c(c3)C(C)(C)c3ccccc3-4)ccc1-2 ; CC(C)(C)c1ccc2c(c1)C(c1ccccc1)(c1ccccc1)c1cc(N(c3ccc4c(c3)C(C)(C)c3ccccc3-4)c3ccccc3-c3ccccc3)ccc1-2]

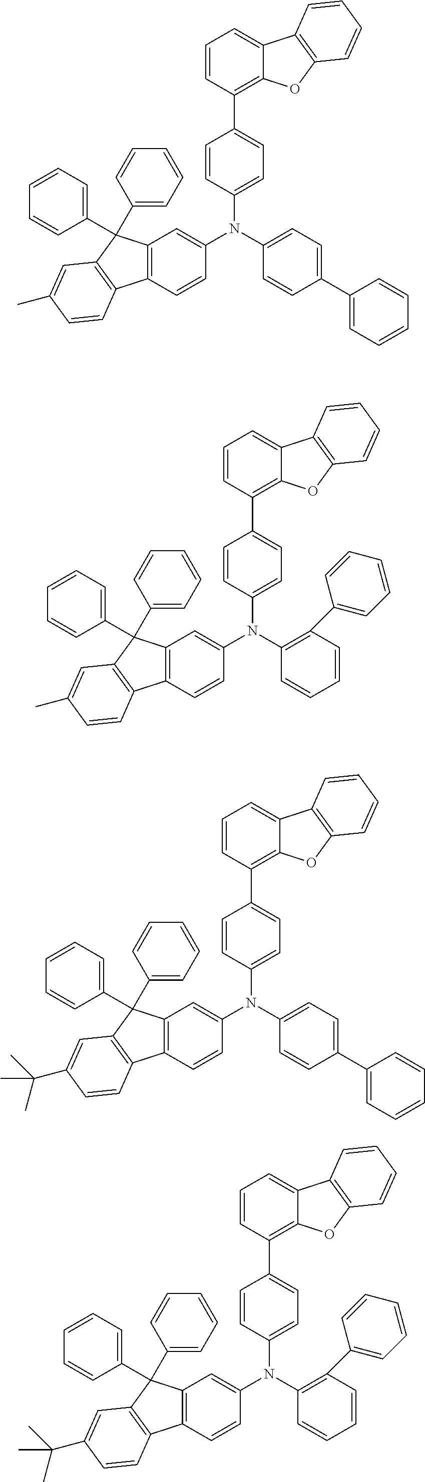

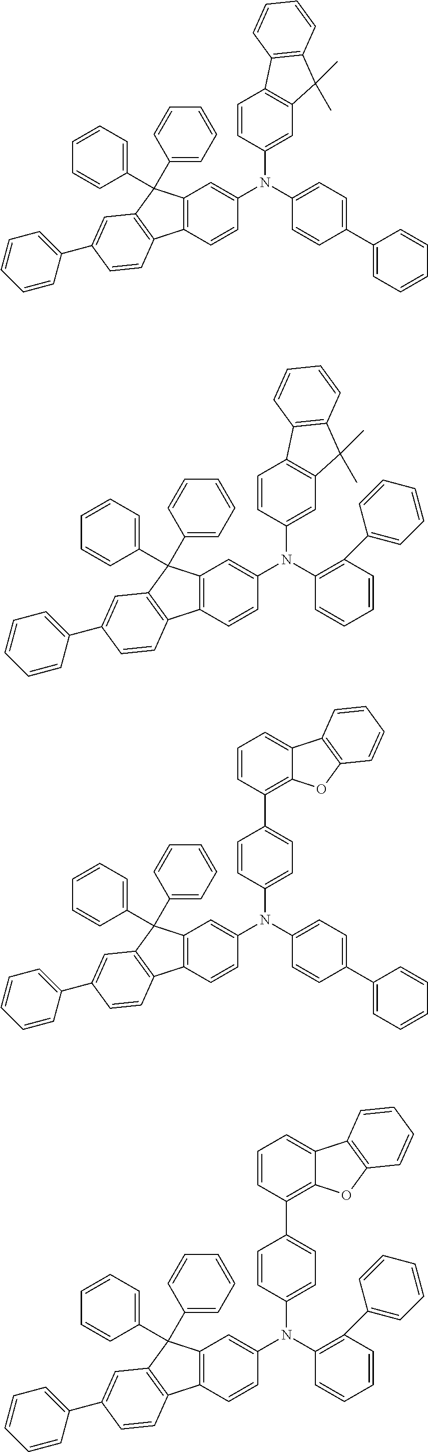 [SMILES: CC1(C)c2ccccc2-c2ccc(N(c3ccc(-c4ccccc4)cc3)c3ccc4c(c3)C(c3ccccc3)(c3ccccc3)c3cc(-c5ccccc5)ccc3-4)cc21 ; CC1(C)c2ccccc2-c2ccc(N(c3ccc4c(c3)C(c3ccccc3)(c3ccccc3)c3cc(-c5ccccc5)ccc3-4)c3ccccc3-c3ccccc3)cc21 ; c1ccc(-c2ccc(N(c3ccc(-c4cccc5c4oc4ccccc45)cc3)c3ccc4c(c3)C(c3ccccc3)(c3ccccc3)c3cc(-c5ccccc5)ccc3-4)cc2)cc1 ; c1ccc(-c2ccc3c(c2)C(c2ccccc2)(c2ccccc2)c2cc(N(c4ccc(-c5cccc6c5oc5ccccc56)cc4)c4ccccc4-c4ccccc4)ccc2-3)cc1]

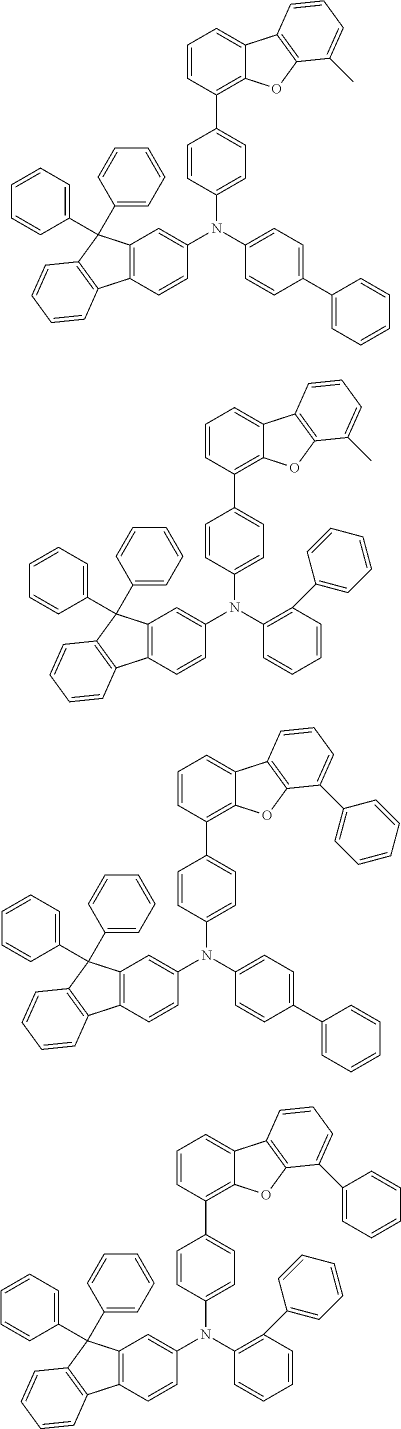

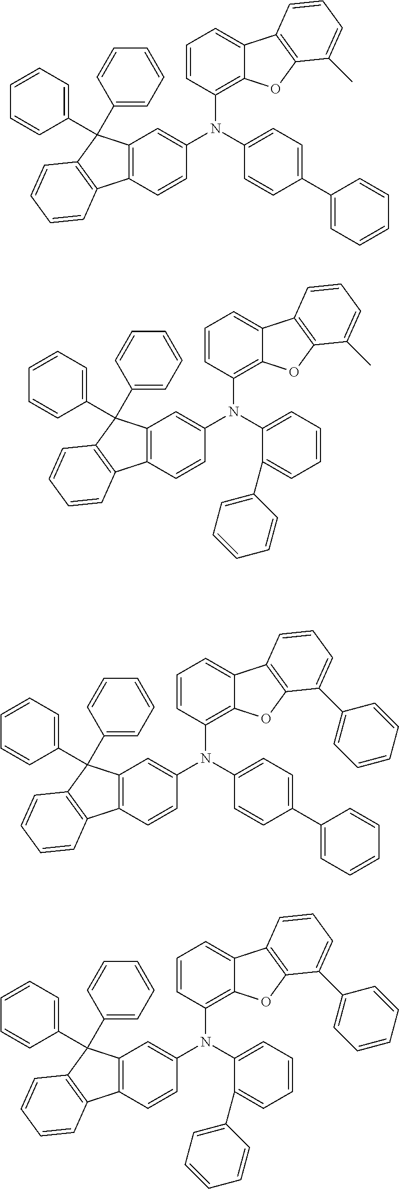

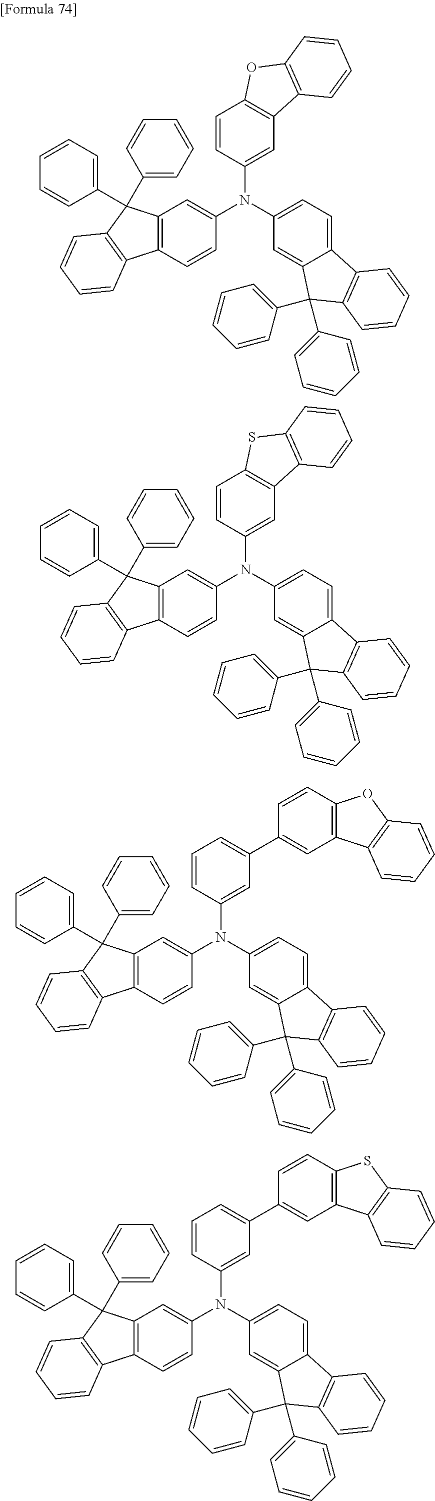

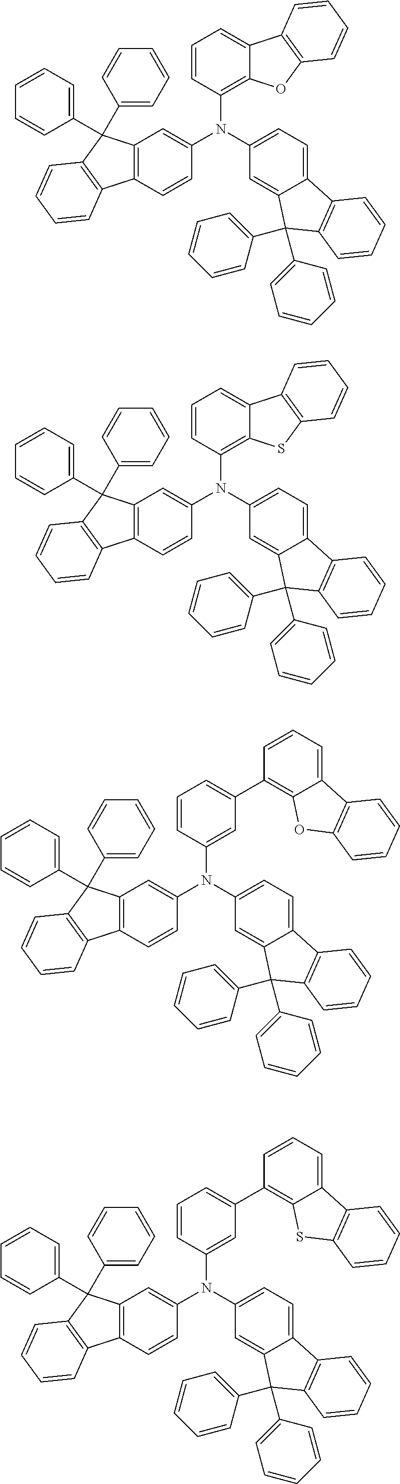

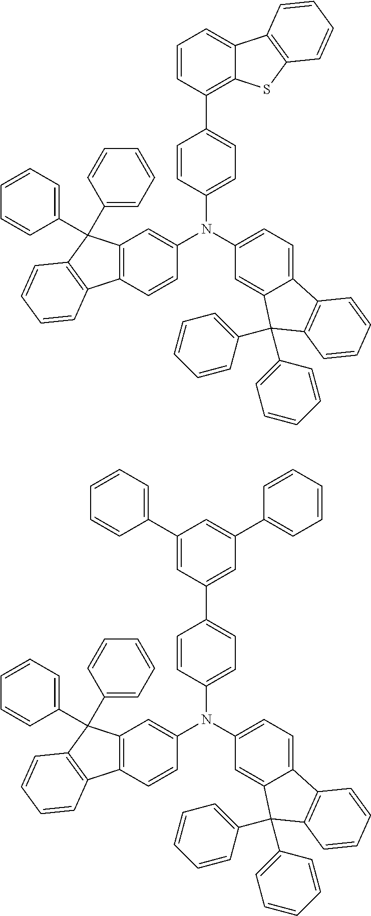

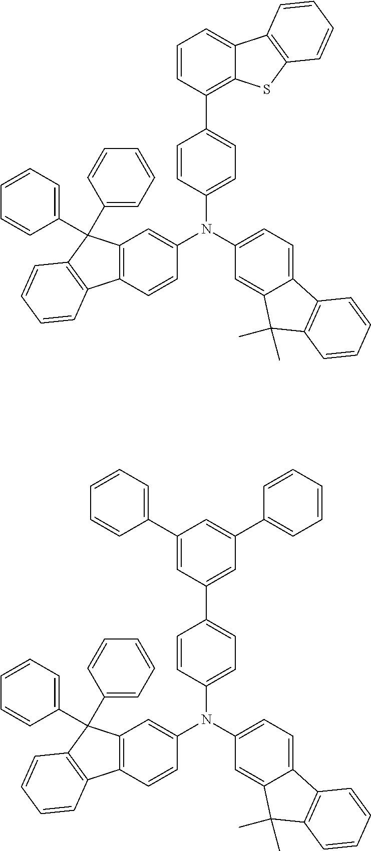 [SMILES: CC1(C)c2ccccc2-c2ccc(N(c3ccc(-c4cccc5c4sc4ccccc45)cc3)c3ccc4c(c3)C(c3ccccc3)(c3ccccc3)c3ccccc3-4)cc21 ; CC1(C)c2ccccc2-c2ccc(N(c3ccc(-c4cc(-c5ccccc5)cc(-c5ccccc5)c4)cc3)c3ccc4c(c3)C(c3ccccc3)(c3ccccc3)c3ccccc3-4)cc21]

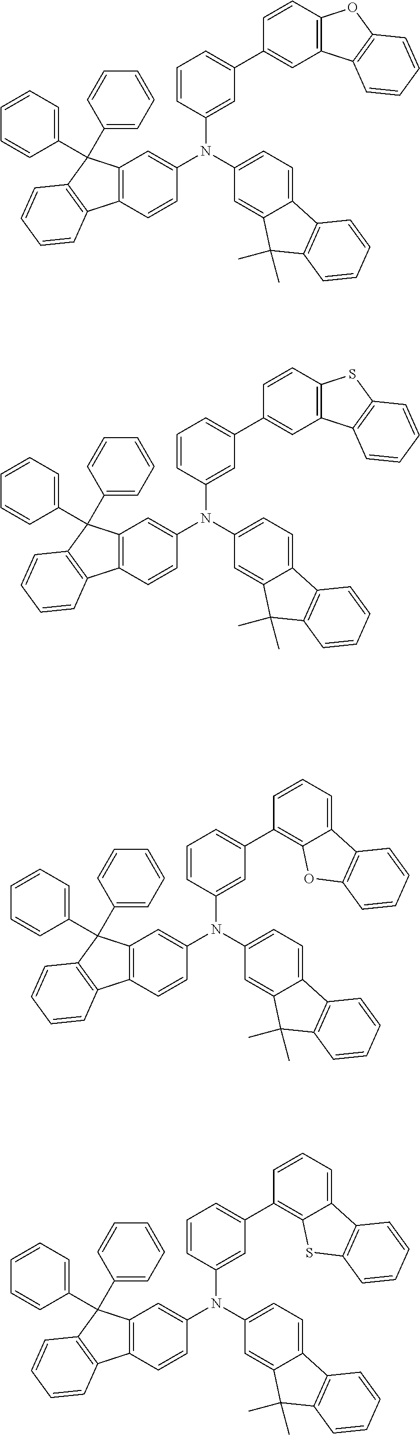 [SMILES: CC1(C)c2ccccc2-c2ccc(N(c3cccc(-c4ccc5oc6ccccc6c5c4)c3)c3ccc4c(c3)C(c3ccccc3)(c3ccccc3)c3ccccc3-4)cc21 ; CC1(C)c2ccccc2-c2ccc(N(c3cccc(-c4ccc5sc6ccccc6c5c4)c3)c3ccc4c(c3)C(c3ccccc3)(c3ccccc3)c3ccccc3-4)cc21 ; CC1(C)c2ccccc2-c2ccc(N(c3cccc(-c4cccc5c4oc4ccccc45)c3)c3ccc4c(c3)C(c3ccccc3)(c3ccccc3)c3ccccc3-4)cc21 ; CC1(C)c2ccccc2-c2ccc(N(c3cccc(-c4cccc5c4sc4ccccc45)c3)c3ccc4c(c3)C(c3ccccc3)(c3ccccc3)c3ccccc3-4)cc21]

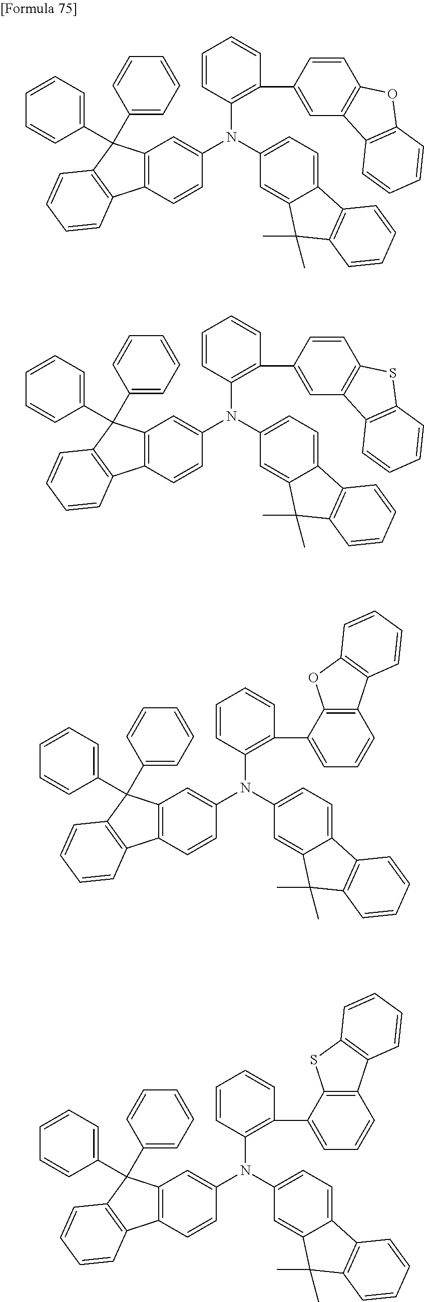 [SMILES: CC1(C)c2ccccc2-c2ccc(N(c3ccc4c(c3)C(c3ccccc3)(c3ccccc3)c3ccccc3-4)c3ccccc3-c3ccc4oc5ccccc5c4c3)cc21 ; CC1(C)c2ccccc2-c2ccc(N(c3ccc4c(c3)C(c3ccccc3)(c3ccccc3)c3ccccc3-4)c3ccccc3-c3ccc4sc5ccccc5c4c3)cc21 ; CC1(C)c2ccccc2-c2ccc(N(c3ccc4c(c3)C(c3ccccc3)(c3ccccc3)c3ccccc3-4)c3ccccc3-c3cccc4c3oc3ccccc34)cc21 ; CC1(C)c2ccccc2-c2ccc(N(c3ccc4c(c3)C(c3ccccc3)(c3ccccc3)c3ccccc3-4)c3ccccc3-c3cccc4c3sc3ccccc34)cc21]

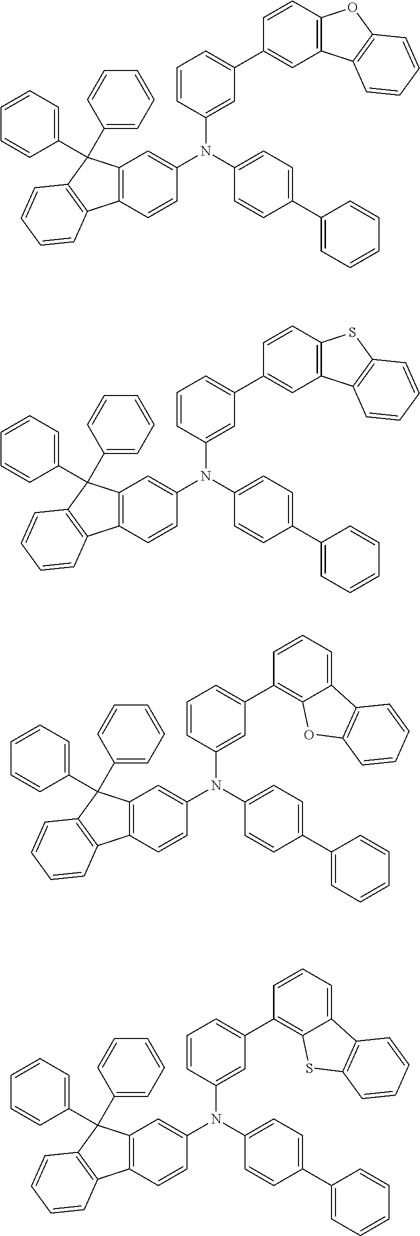 [SMILES: c1ccc(-c2ccc(N(c3cccc(-c4ccc5oc6ccccc6c5c4)c3)c3ccc4c(c3)C(c3ccccc3)(c3ccccc3)c3ccccc3-4)cc2)cc1 ; c1ccc(-c2ccc(N(c3cccc(-c4ccc5sc6ccccc6c5c4)c3)c3ccc4c(c3)C(c3ccccc3)(c3ccccc3)c3ccccc3-4)cc2)cc1 ; c1ccc(-c2ccc(N(c3cccc(-c4cccc5c4oc4ccccc45)c3)c3ccc4c(c3)C(c3ccccc3)(c3ccccc3)c3ccccc3-4)cc2)cc1 ; c1ccc(-c2ccc(N(c3cccc(-c4cccc5c4sc4ccccc45)c3)c3ccc4c(c3)C(c3ccccc3)(c3ccccc3)c3ccccc3-4)cc2)cc1]

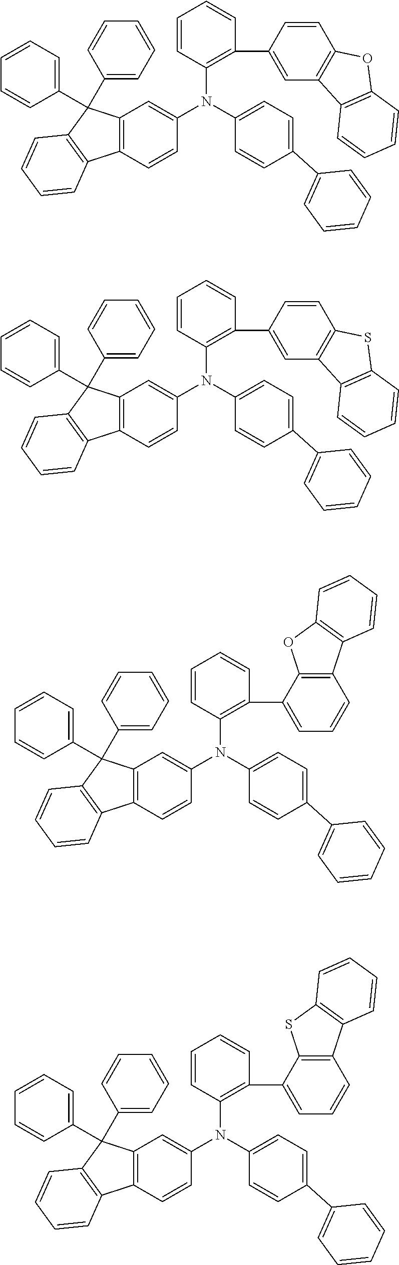 [SMILES: c1ccc(-c2ccc(N(c3ccc4c(c3)C(c3ccccc3)(c3ccccc3)c3ccccc3-4)c3ccccc3-c3ccc4oc5ccccc5c4c3)cc2)cc1 ; c1ccc(-c2ccc(N(c3ccc4c(c3)C(c3ccccc3)(c3ccccc3)c3ccccc3-4)c3ccccc3-c3ccc4sc5ccccc5c4c3)cc2)cc1 ; c1ccc(-c2ccc(N(c3ccc4c(c3)C(c3ccccc3)(c3ccccc3)c3ccccc3-4)c3ccccc3-c3cccc4c3oc3ccccc34)cc2)cc1 ; c1ccc(-c2ccc(N(c3ccc4c(c3)C(c3ccccc3)(c3ccccc3)c3ccccc3-4)c3ccccc3-c3cccc4c3sc3ccccc34)cc2)cc1]

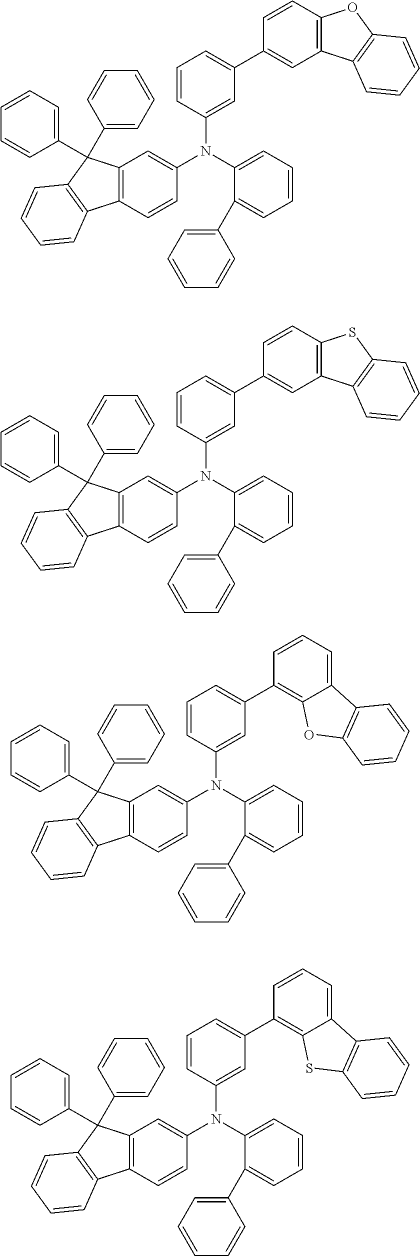 [SMILES: c1ccc(-c2ccccc2N(c2cccc(-c3ccc4oc5ccccc5c4c3)c2)c2ccc3c(c2)C(c2ccccc2)(c2ccccc2)c2ccccc2-3)cc1 ; c1ccc(-c2ccccc2N(c2cccc(-c3ccc4sc5ccccc5c4c3)c2)c2ccc3c(c2)C(c2ccccc2)(c2ccccc2)c2ccccc2-3)cc1 ; c1ccc(-c2ccccc2N(c2cccc(-c3cccc4c3oc3ccccc34)c2)c2ccc3c(c2)C(c2ccccc2)(c2ccccc2)c2ccccc2-3)cc1 ; c1ccc(-c2ccccc2N(c2cccc(-c3cccc4c3sc3ccccc34)c2)c2ccc3c(c2)C(c2ccccc2)(c2ccccc2)c2ccccc2-3)cc1]

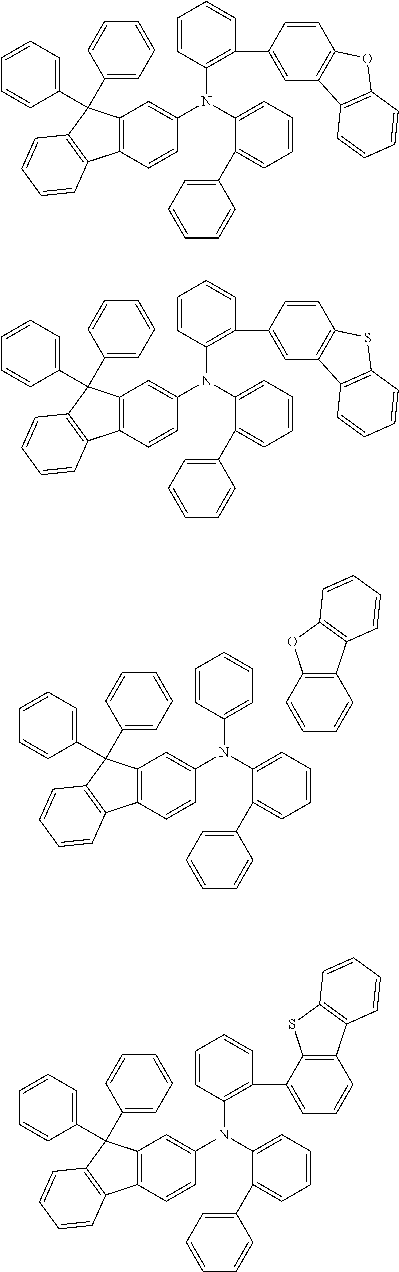

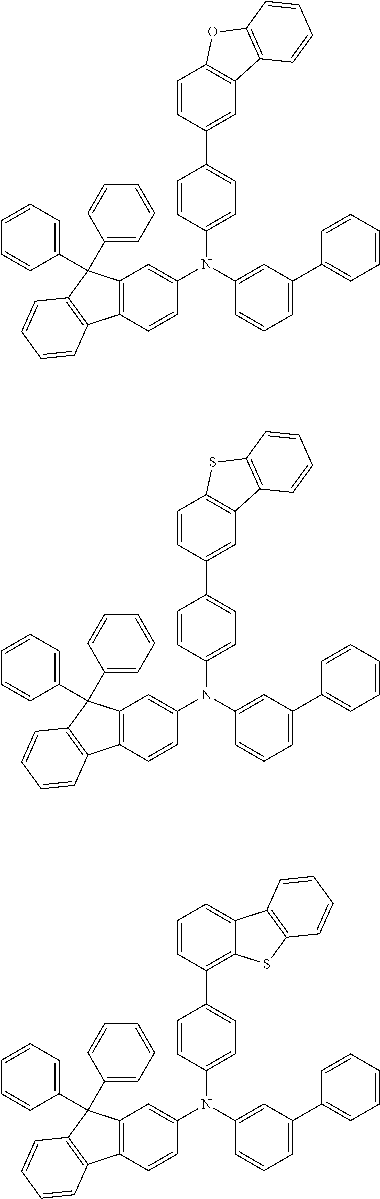 [SMILES: c1ccc(-c2cccc(N(c3ccc(-c4ccc5oc6ccccc6c5c4)cc3)c3ccc4c(c3)C(c3ccccc3)(c3ccccc3)c3ccccc3-4)c2)cc1 ; c1ccc(-c2cccc(N(c3ccc(-c4ccc5sc6ccccc6c5c4)cc3)c3ccc4c(c3)C(c3ccccc3)(c3ccccc3)c3ccccc3-4)c2)cc1 ; c1ccc(-c2cccc(N(c3ccc(-c4cccc5c4sc4ccccc45)cc3)c3ccc4c(c3)C(c3ccccc3)(c3ccccc3)c3ccccc3-4)c2)cc1]

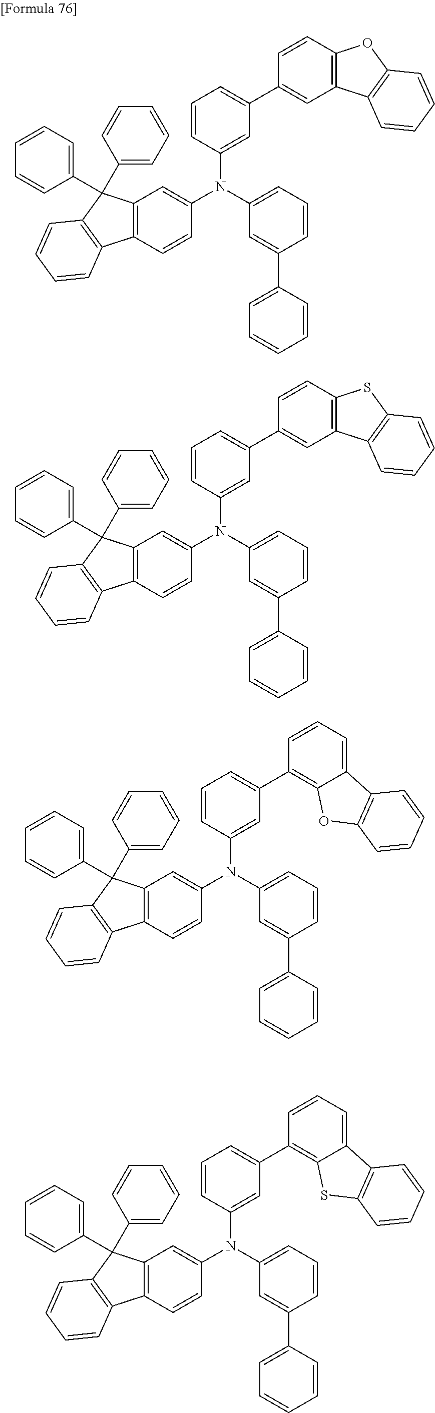

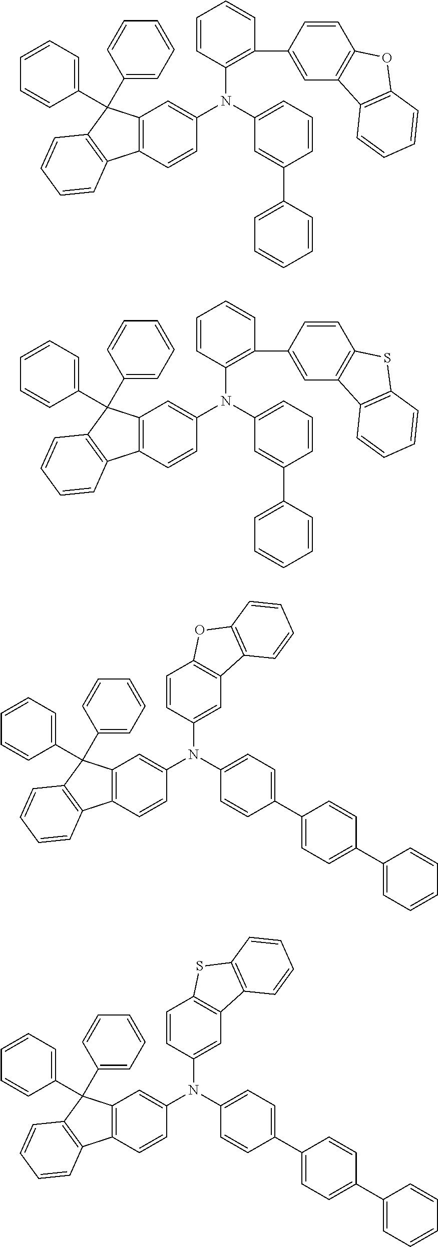

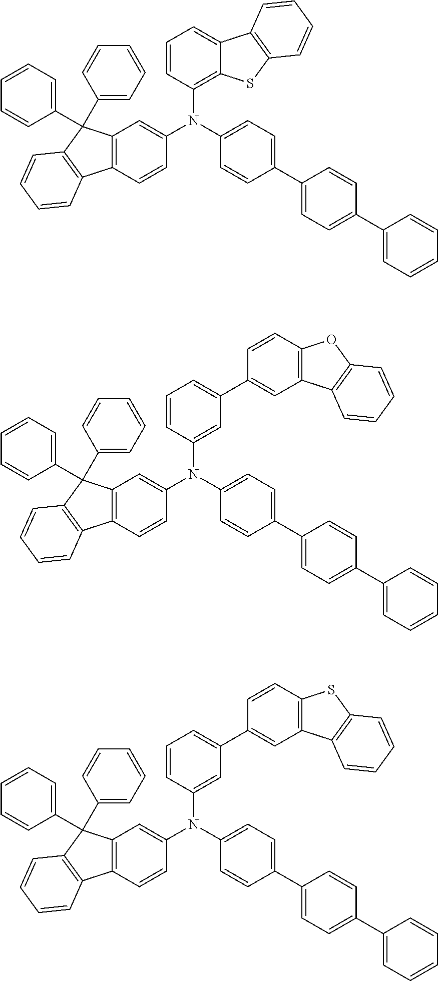 [SMILES: c1ccc(-c2ccc(-c3ccc(N(c4ccc5c(c4)C(c4ccccc4)(c4ccccc4)c4ccccc4-5)c4cccc5c4sc4ccccc45)cc3)cc2)cc1 ; c1ccc(-c2ccc(-c3ccc(N(c4cccc(-c5ccc6oc7ccccc7c6c5)c4)c4ccc5c(c4)C(c4ccccc4)(c4ccccc4)c4ccccc4-5)cc3)cc2)cc1 ; c1ccc(-c2ccc(-c3ccc(N(c4cccc(-c5ccc6sc7ccccc7c6c5)c4)c4ccc5c(c4)C(c4ccccc4)(c4ccccc4)c4ccccc4-5)cc3)cc2)cc1]

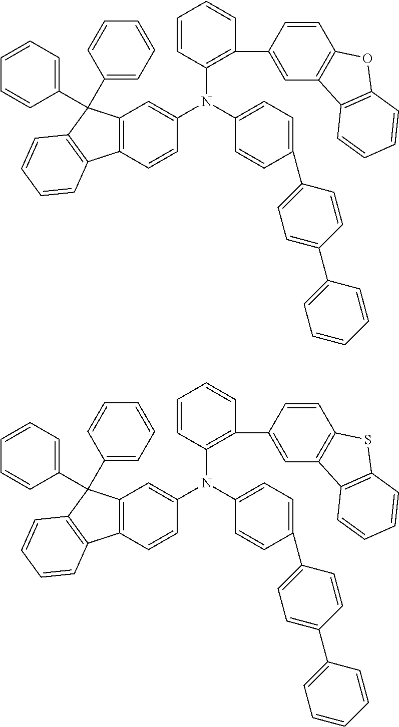 [SMILES: c1ccc(-c2ccc(-c3ccc(N(c4ccc5c(c4)C(c4ccccc4)(c4ccccc4)c4ccccc4-5)c4ccccc4-c4ccc5oc6ccccc6c5c4)cc3)cc2)cc1 ; c1ccc(-c2ccc(-c3ccc(N(c4ccc5c(c4)C(c4ccccc4)(c4ccccc4)c4ccccc4-5)c4ccccc4-c4ccc5sc6ccccc6c5c4)cc3)cc2)cc1]

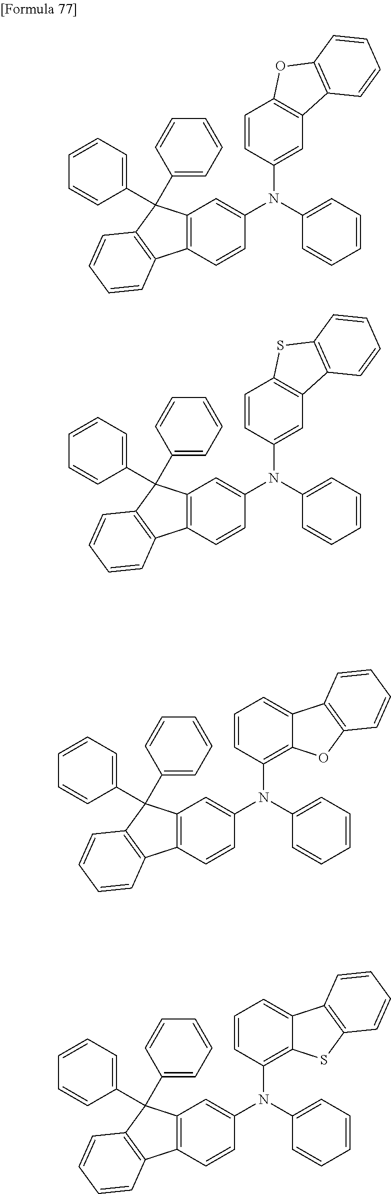 [SMILES: c1ccc(N(c2ccc3c(c2)C(c2ccccc2)(c2ccccc2)c2ccccc2-3)c2ccc3oc4ccccc4c3c2)cc1 ; c1ccc(N(c2ccc3c(c2)C(c2ccccc2)(c2ccccc2)c2ccccc2-3)c2ccc3sc4ccccc4c3c2)cc1 ; c1ccc(N(c2ccc3c(c2)C(c2ccccc2)(c2ccccc2)c2ccccc2-3)c2cccc3c2oc2ccccc23)cc1 ; c1ccc(N(c2ccc3c(c2)C(c2ccccc2)(c2ccccc2)c2ccccc2-3)c2cccc3c2sc2ccccc23)cc1]

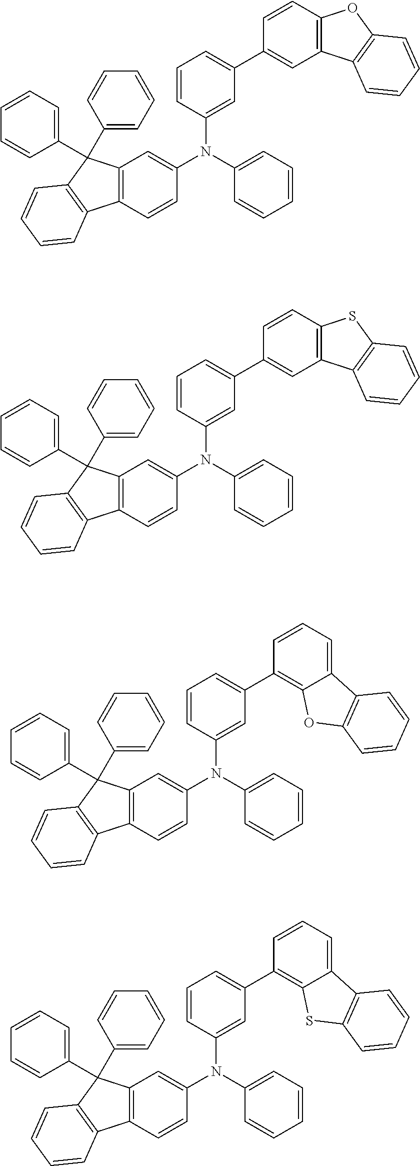 [SMILES: c1ccc(N(c2cccc(-c3ccc4oc5ccccc5c4c3)c2)c2ccc3c(c2)C(c2ccccc2)(c2ccccc2)c2ccccc2-3)cc1 ; c1ccc(N(c2cccc(-c3ccc4sc5ccccc5c4c3)c2)c2ccc3c(c2)C(c2ccccc2)(c2ccccc2)c2ccccc2-3)cc1 ; c1ccc(N(c2cccc(-c3cccc4c3oc3ccccc34)c2)c2ccc3c(c2)C(c2ccccc2)(c2ccccc2)c2ccccc2-3)cc1 ; c1ccc(N(c2cccc(-c3cccc4c3sc3ccccc34)c2)c2ccc3c(c2)C(c2ccccc2)(c2ccccc2)c2ccccc2-3)cc1]

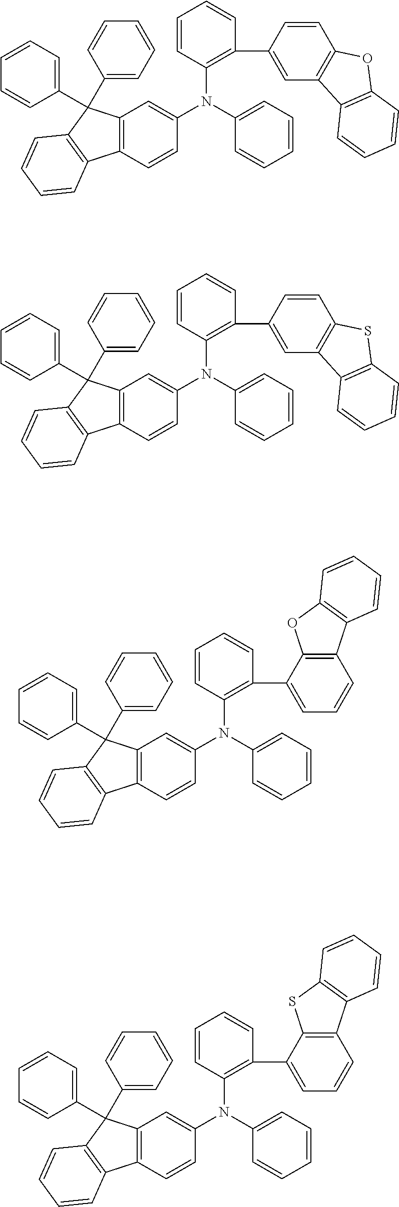 [SMILES: c1ccc(N(c2ccc3c(c2)C(c2ccccc2)(c2ccccc2)c2ccccc2-3)c2ccccc2-c2ccc3oc4ccccc4c3c2)cc1 ; c1ccc(N(c2ccc3c(c2)C(c2ccccc2)(c2ccccc2)c2ccccc2-3)c2ccccc2-c2ccc3sc4ccccc4c3c2)cc1 ; c1ccc(N(c2ccc3c(c2)C(c2ccccc2)(c2ccccc2)c2ccccc2-3)c2ccccc2-c2cccc3c2oc2ccccc23)cc1 ; c1ccc(N(c2ccc3c(c2)C(c2ccccc2)(c2ccccc2)c2ccccc2-3)c2ccccc2-c2cccc3c2sc2ccccc23)cc1]

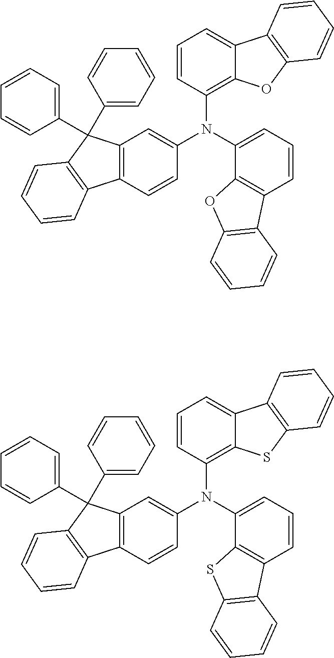 [SMILES: c1ccc(C2(c3ccccc3)c3ccccc3-c3ccc(N(c4cccc5c4oc4ccccc45)c4cccc5c4oc4ccccc45)cc32)cc1 ; c1ccc(C2(c3ccccc3)c3ccccc3-c3ccc(N(c4cccc5c4sc4ccccc45)c4cccc5c4sc4ccccc45)cc32)cc1]

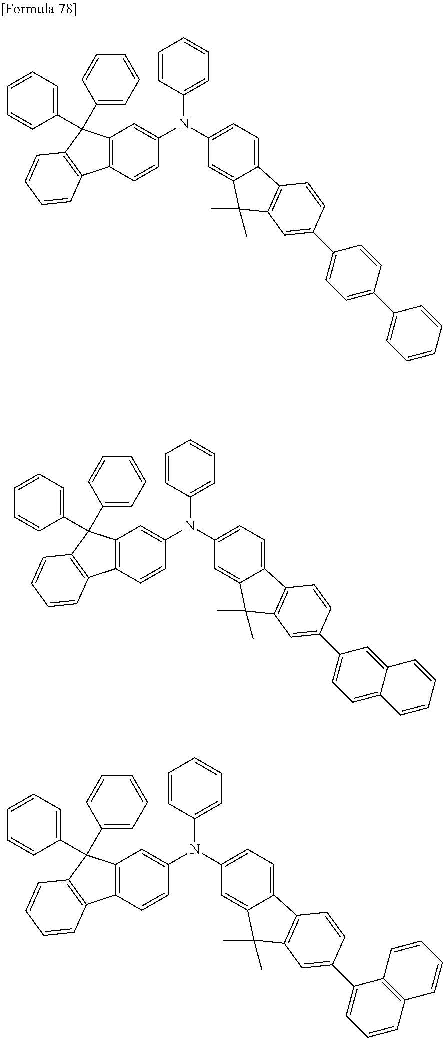 [SMILES: CC1(C)c2cc(-c3ccc(-c4ccccc4)cc3)ccc2-c2ccc(N(c3ccccc3)c3ccc4c(c3)C(c3ccccc3)(c3ccccc3)c3ccccc3-4)cc21 ; CC1(C)c2cc(-c3ccc4ccccc4c3)ccc2-c2ccc(N(c3ccccc3)c3ccc4c(c3)C(c3ccccc3)(c3ccccc3)c3ccccc3-4)cc21 ; CC1(C)c2cc(-c3cccc4ccccc34)ccc2-c2ccc(N(c3ccccc3)c3ccc4c(c3)C(c3ccccc3)(c3ccccc3)c3ccccc3-4)cc21]

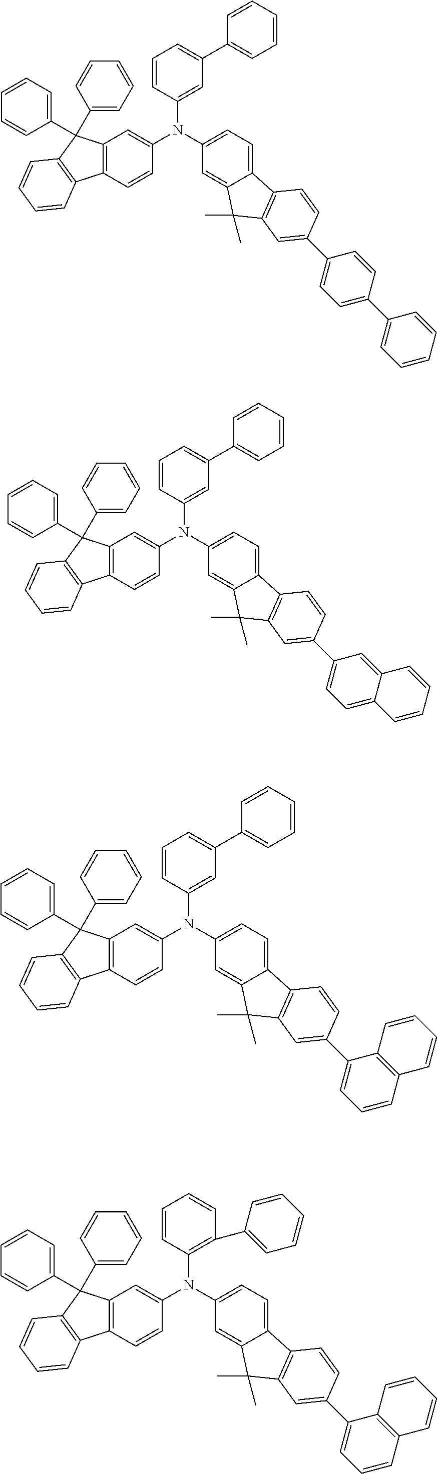 [SMILES: CC1(C)c2cc(-c3ccc(-c4ccccc4)cc3)ccc2-c2ccc(N(c3cccc(-c4ccccc4)c3)c3ccc4c(c3)C(c3ccccc3)(c3ccccc3)c3ccccc3-4)cc21 ; CC1(C)c2cc(-c3ccc4ccccc4c3)ccc2-c2ccc(N(c3cccc(-c4ccccc4)c3)c3ccc4c(c3)C(c3ccccc3)(c3ccccc3)c3ccccc3-4)cc21 ; CC1(C)c2cc(-c3cccc4ccccc34)ccc2-c2ccc(N(c3cccc(-c4ccccc4)c3)c3ccc4c(c3)C(c3ccccc3)(c3ccccc3)c3ccccc3-4)cc21 ; CC1(C)c2cc(-c3cccc4ccccc34)ccc2-c2ccc(N(c3ccc4c(c3)C(c3ccccc3)(c3ccccc3)c3ccccc3-4)c3ccccc3-c3ccccc3)cc21]

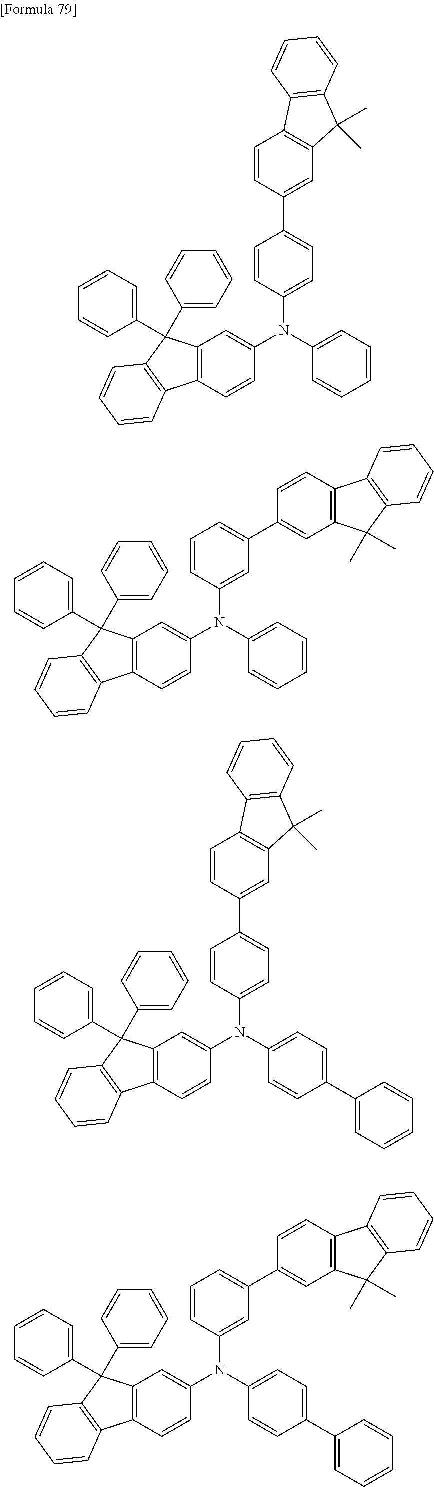 [SMILES: CC1(C)c2ccccc2-c2ccc(-c3ccc(N(c4ccccc4)c4ccc5c(c4)C(c4ccccc4)(c4ccccc4)c4ccccc4-5)cc3)cc21 ; CC1(C)c2ccccc2-c2ccc(-c3cccc(N(c4ccccc4)c4ccc5c(c4)C(c4ccccc4)(c4ccccc4)c4ccccc4-5)c3)cc21 ; CC1(C)c2ccccc2-c2ccc(-c3ccc(N(c4ccc(-c5ccccc5)cc4)c4ccc5c(c4)C(c4ccccc4)(c4ccccc4)c4ccccc4-5)cc3)cc21 ; CC1(C)c2ccccc2-c2ccc(-c3cccc(N(c4ccc(-c5ccccc5)cc4)c4ccc5c(c4)C(c4ccccc4)(c4ccccc4)c4ccccc4-5)c3)cc21]

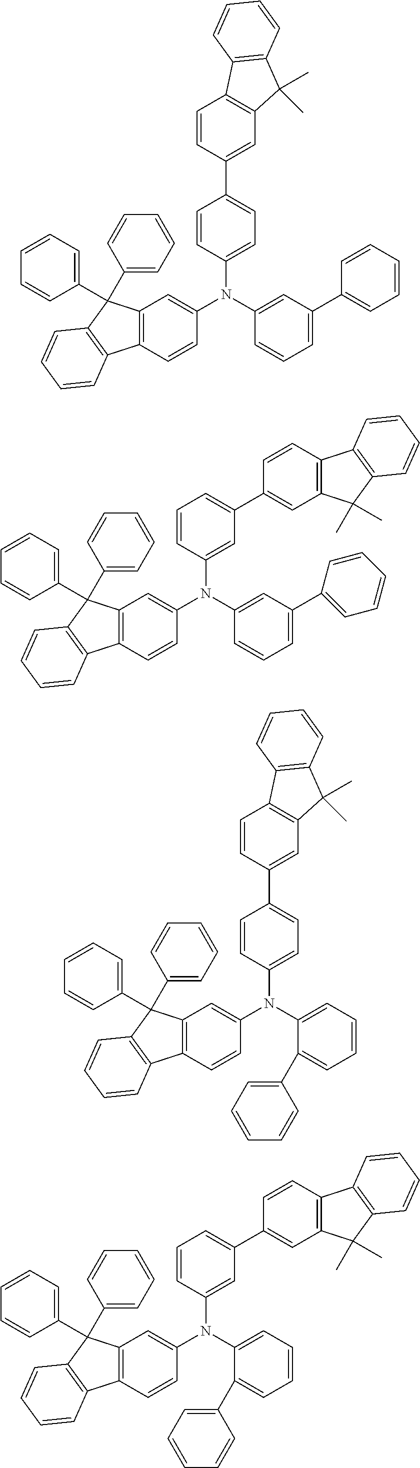

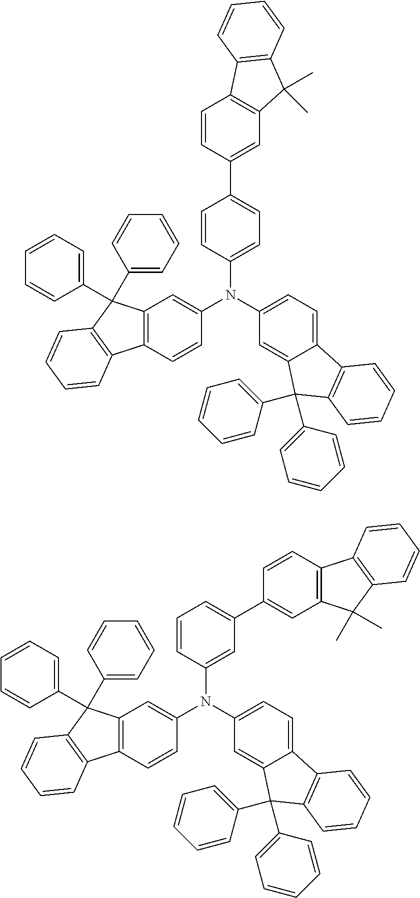 [SMILES: CC1(C)c2ccccc2-c2ccc(-c3ccc(N(c4ccc5c(c4)C(c4ccccc4)(c4ccccc4)c4ccccc4-5)c4ccc5c(c4)C(c4ccccc4)(c4ccccc4)c4ccccc4-5)cc3)cc21 ; CC1(C)c2ccccc2-c2ccc(-c3cccc(N(c4ccc5c(c4)C(c4ccccc4)(c4ccccc4)c4ccccc4-5)c4ccc5c(c4)C(c4ccccc4)(c4ccccc4)c4ccccc4-5)c3)cc21]

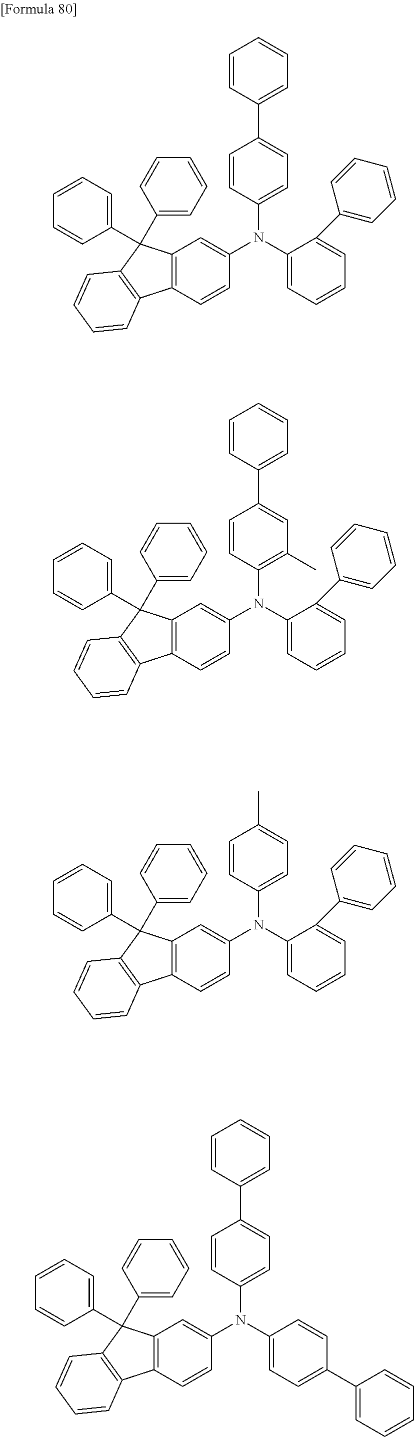 [SMILES: c1ccc(-c2ccc(N(c3ccc4c(c3)C(c3ccccc3)(c3ccccc3)c3ccccc3-4)c3ccccc3-c3ccccc3)cc2)cc1 ; Cc1cc(-c2ccccc2)ccc1N(c1ccc2c(c1)C(c1ccccc1)(c1ccccc1)c1ccccc1-2)c1ccccc1-c1ccccc1 ; Cc1ccc(N(c2ccc3c(c2)C(c2ccccc2)(c2ccccc2)c2ccccc2-3)c2ccccc2-c2ccccc2)cc1 ; c1ccc(-c2ccc(N(c3ccc(-c4ccccc4)cc3)c3ccc4c(c3)C(c3ccccc3)(c3ccccc3)c3ccccc3-4)cc2)cc1]

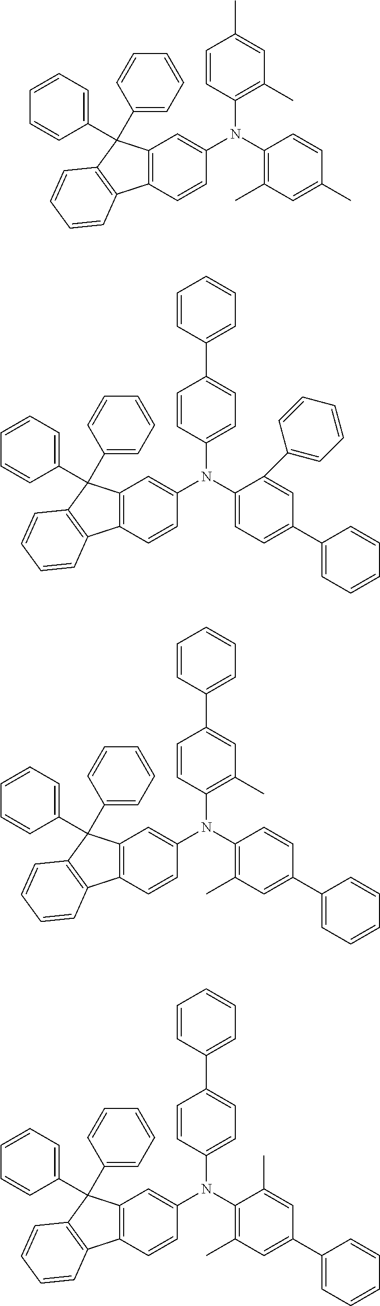 [SMILES: Cc1ccc(N(c2ccc3c(c2)C(c2ccccc2)(c2ccccc2)c2ccccc2-3)c2ccc(C)cc2C)c(C)c1 ; c1ccc(-c2ccc(N(c3ccc4c(c3)C(c3ccccc3)(c3ccccc3)c3ccccc3-4)c3ccc(-c4ccccc4)cc3-c3ccccc3)cc2)cc1 ; Cc1cc(-c2ccccc2)ccc1N(c1ccc2c(c1)C(c1ccccc1)(c1ccccc1)c1ccccc1-2)c1ccc(-c2ccccc2)cc1C ; Cc1cc(-c2ccccc2)cc(C)c1N(c1ccc(-c2ccccc2)cc1)c1ccc2c(c1)C(c1ccccc1)(c1ccccc1)c1ccccc1-2]

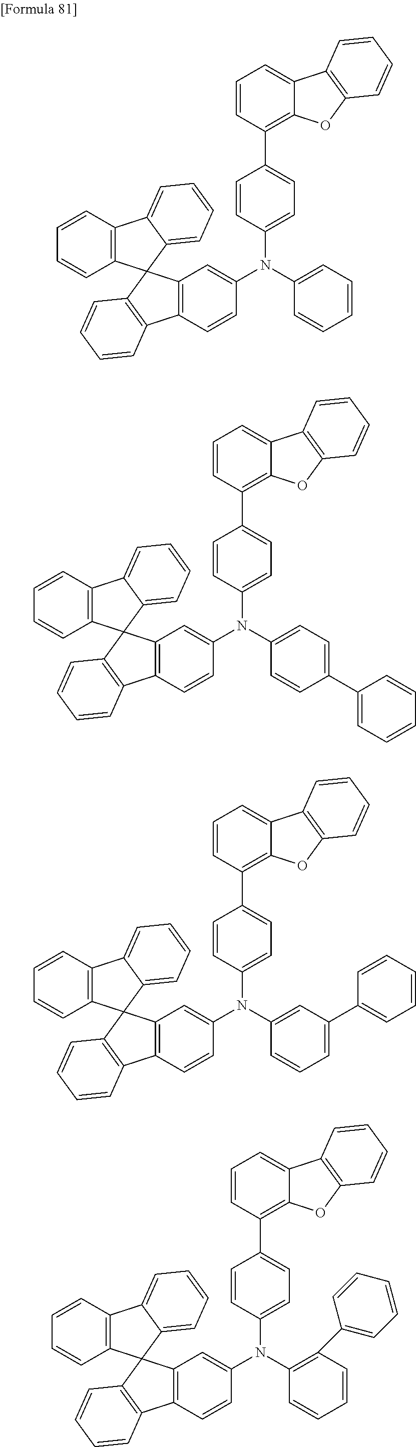 [SMILES: c1ccc(N(c2ccc(-c3cccc4c3oc3ccccc34)cc2)c2ccc3c(c2)C2(c4ccccc4-c4ccccc42)c2ccccc2-3)cc1 ; c1ccc(-c2ccc(N(c3ccc(-c4cccc5c4oc4ccccc45)cc3)c3ccc4c(c3)C3(c5ccccc5-c5ccccc53)c3ccccc3-4)cc2)cc1 ; c1ccc(-c2cccc(N(c3ccc(-c4cccc5c4oc4ccccc45)cc3)c3ccc4c(c3)C3(c5ccccc5-c5ccccc53)c3ccccc3-4)c2)cc1 ; c1ccc(-c2ccccc2N(c2ccc(-c3cccc4c3oc3ccccc34)cc2)c2ccc3c(c2)C2(c4ccccc4-c4ccccc42)c2ccccc2-3)cc1]

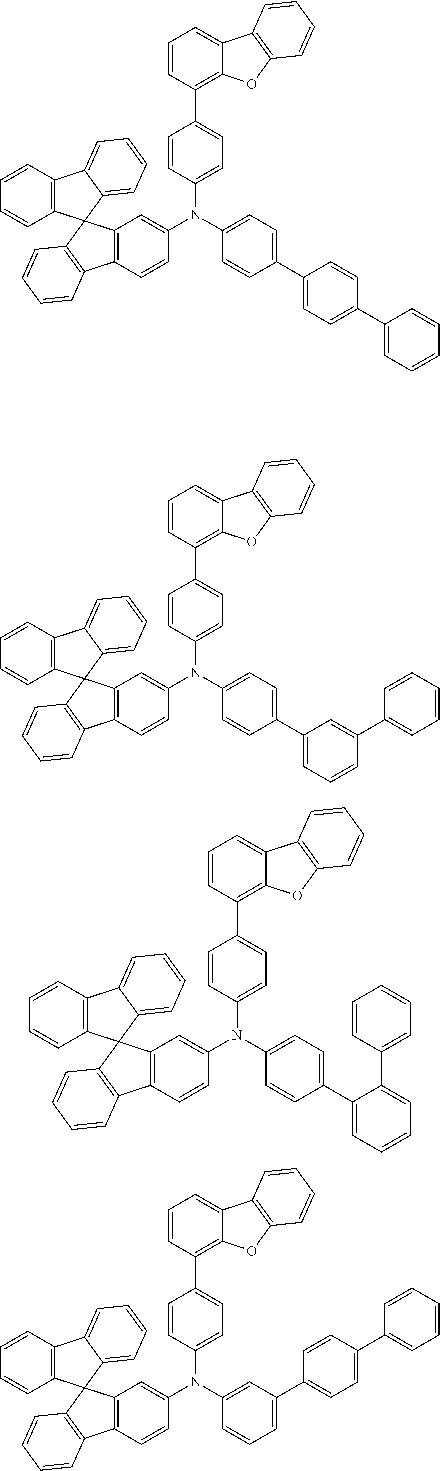

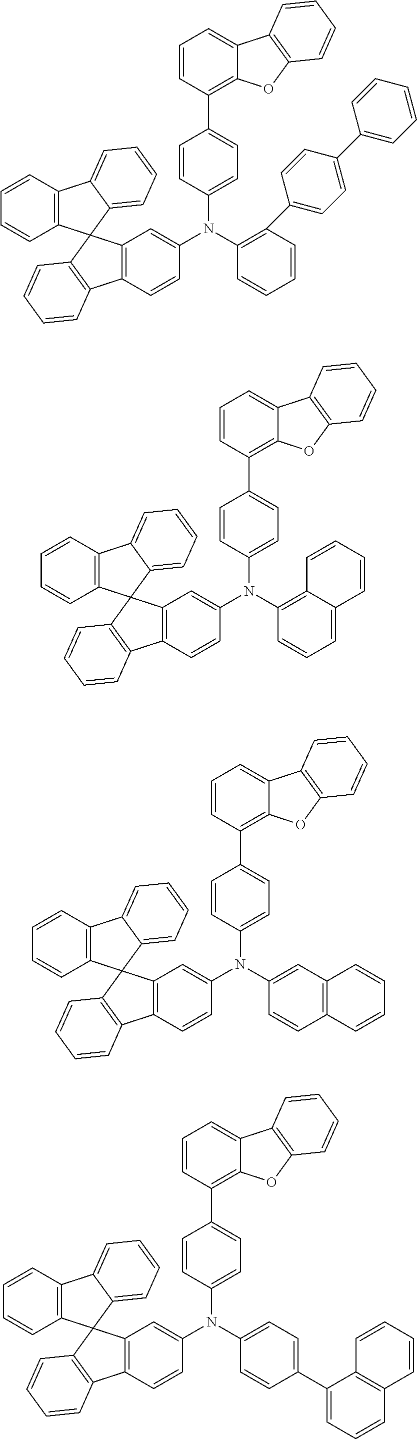 [SMILES: c1ccc(-c2ccc(-c3ccccc3N(c3ccc(-c4cccc5c4oc4ccccc45)cc3)c3ccc4c(c3)C3(c5ccccc5-c5ccccc53)c3ccccc3-4)cc2)cc1 ; c1ccc2c(c1)-c1ccccc1C21c2ccccc2-c2ccc(N(c3ccc(-c4cccc5c4oc4ccccc45)cc3)c3cccc4ccccc34)cc21 ; c1ccc2c(c1)-c1ccccc1C21c2ccccc2-c2ccc(N(c3ccc(-c4cccc5c4oc4ccccc45)cc3)c3ccc4ccccc4c3)cc21 ; c1ccc2c(c1)-c1ccccc1C21c2ccccc2-c2ccc(N(c3ccc(-c4cccc5ccccc45)cc3)c3ccc(-c4cccc5c4oc4ccccc45)cc3)cc21]

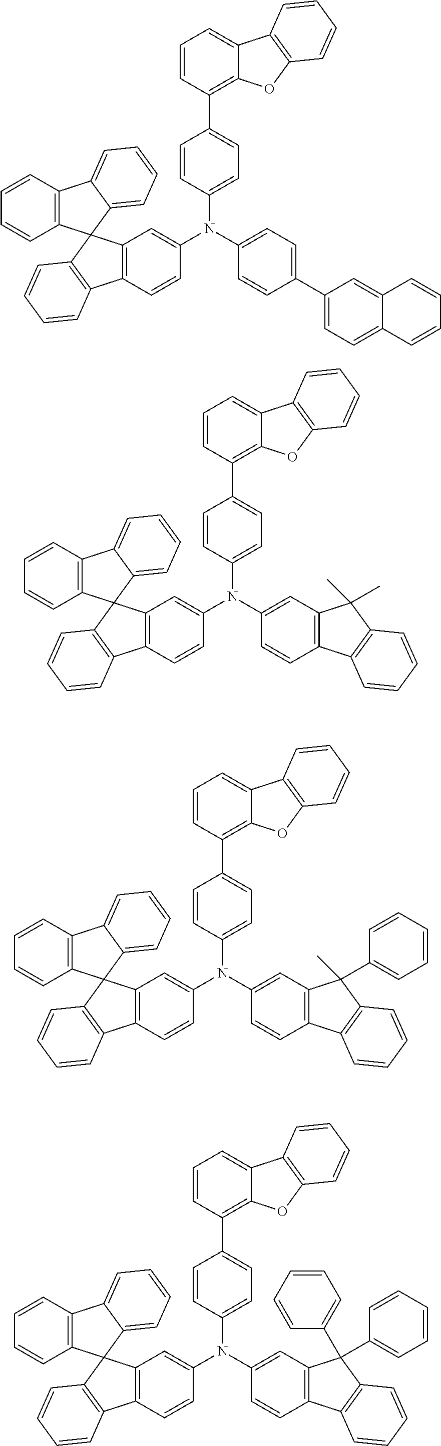

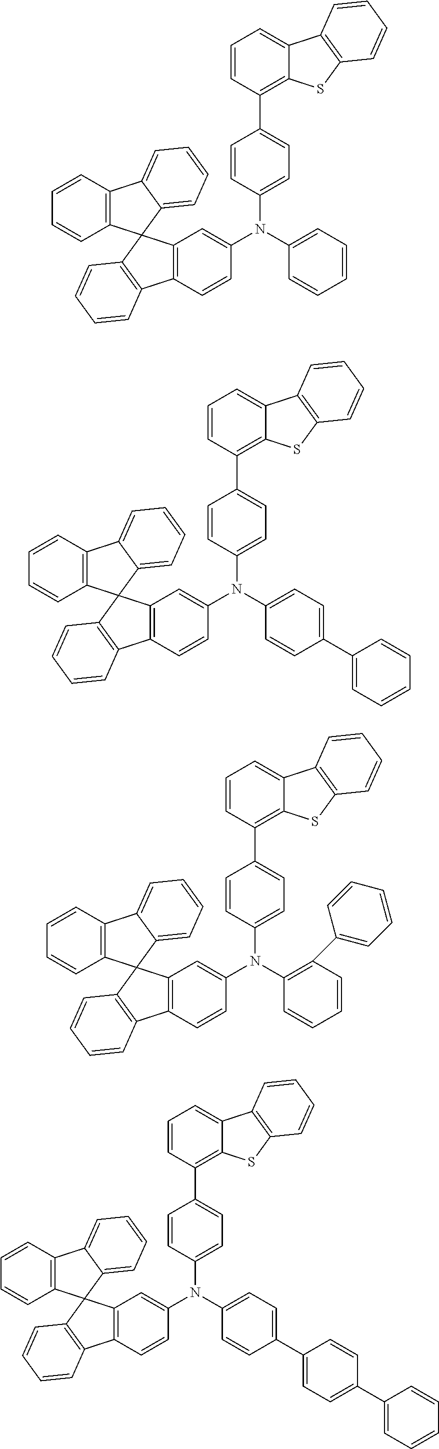

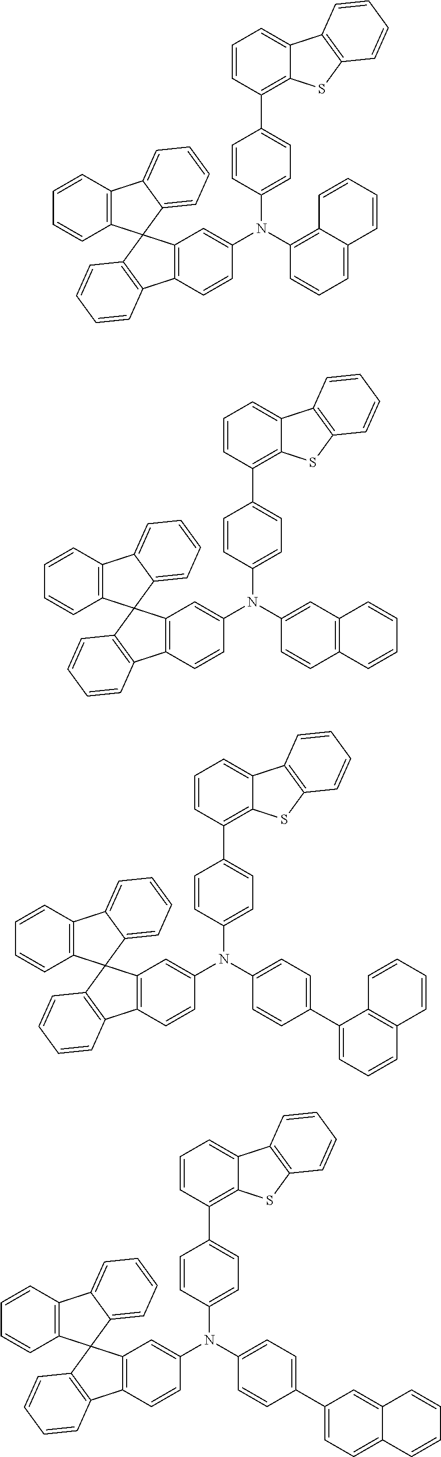 [SMILES: c1ccc2c(c1)-c1ccccc1C21c2ccccc2-c2ccc(N(c3ccc(-c4cccc5c4sc4ccccc45)cc3)c3cccc4ccccc34)cc21 ; c1ccc2c(c1)-c1ccccc1C21c2ccccc2-c2ccc(N(c3ccc(-c4cccc5c4sc4ccccc45)cc3)c3ccc4ccccc4c3)cc21 ; c1ccc2c(c1)-c1ccccc1C21c2ccccc2-c2ccc(N(c3ccc(-c4cccc5ccccc45)cc3)c3ccc(-c4cccc5c4sc4ccccc45)cc3)cc21 ; c1ccc2c(c1)-c1ccccc1C21c2ccccc2-c2ccc(N(c3ccc(-c4ccc5ccccc5c4)cc3)c3ccc(-c4cccc5c4sc4ccccc45)cc3)cc21]

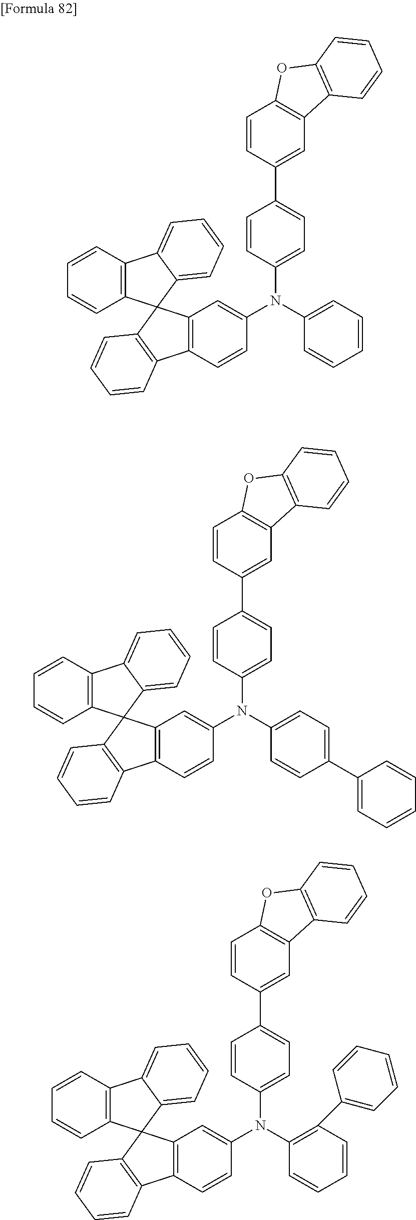 [SMILES: c1ccc(N(c2ccc(-c3ccc4oc5ccccc5c4c3)cc2)c2ccc3c(c2)C2(c4ccccc4-c4ccccc42)c2ccccc2-3)cc1 ; c1ccc(-c2ccc(N(c3ccc(-c4ccc5oc6ccccc6c5c4)cc3)c3ccc4c(c3)C3(c5ccccc5-c5ccccc53)c3ccccc3-4)cc2)cc1 ; c1ccc(-c2ccccc2N(c2ccc(-c3ccc4oc5ccccc5c4c3)cc2)c2ccc3c(c2)C2(c4ccccc4-c4ccccc42)c2ccccc2-3)cc1]

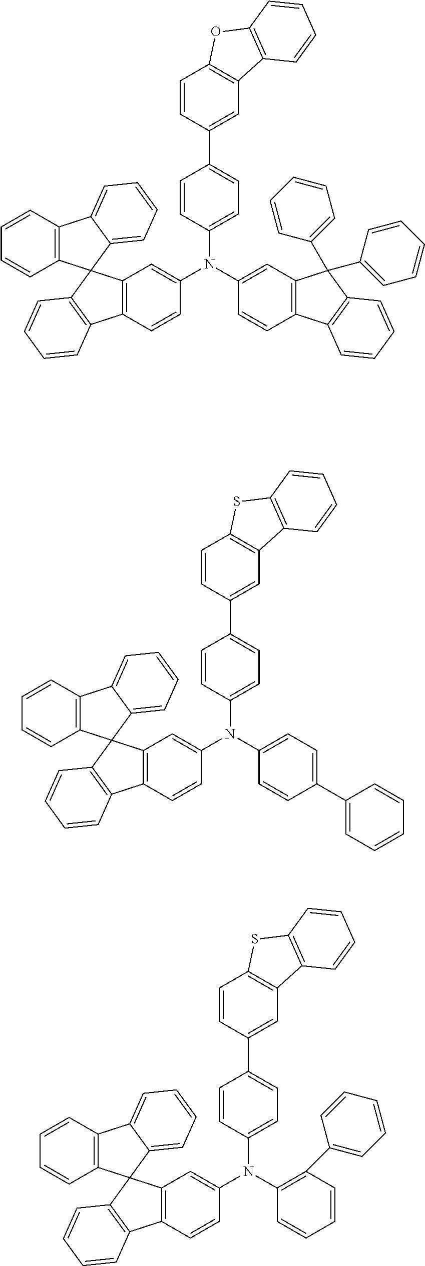 [SMILES: c1ccc(C2(c3ccccc3)c3ccccc3-c3ccc(N(c4ccc(-c5ccc6oc7ccccc7c6c5)cc4)c4ccc5c(c4)C4(c6ccccc6-c6ccccc64)c4ccccc4-5)cc32)cc1 ; c1ccc(-c2ccc(N(c3ccc(-c4ccc5sc6ccccc6c5c4)cc3)c3ccc4c(c3)C3(c5ccccc5-c5ccccc53)c3ccccc3-4)cc2)cc1 ; c1ccc(-c2ccccc2N(c2ccc(-c3ccc4sc5ccccc5c4c3)cc2)c2ccc3c(c2)C2(c4ccccc4-c4ccccc42)c2ccccc2-3)cc1]

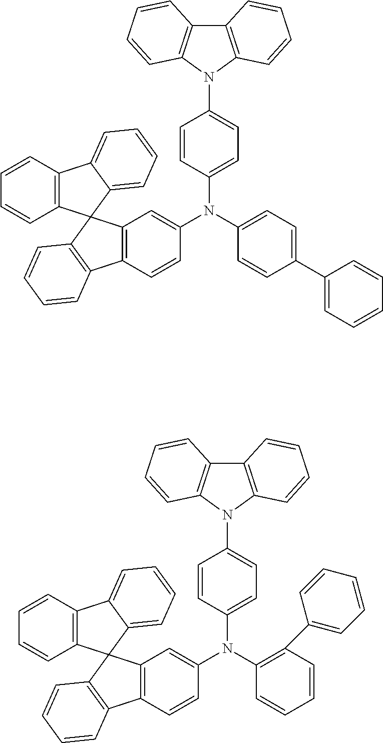 [SMILES: c1ccc(-c2ccc(N(c3ccc(-n4c5ccccc5c5ccccc54)cc3)c3ccc4c(c3)C3(c5ccccc5-c5ccccc53)c3ccccc3-4)cc2)cc1 ; c1ccc(-c2ccccc2N(c2ccc(-n3c4ccccc4c4ccccc43)cc2)c2ccc3c(c2)C2(c4ccccc4-c4ccccc42)c2ccccc2-3)cc1]

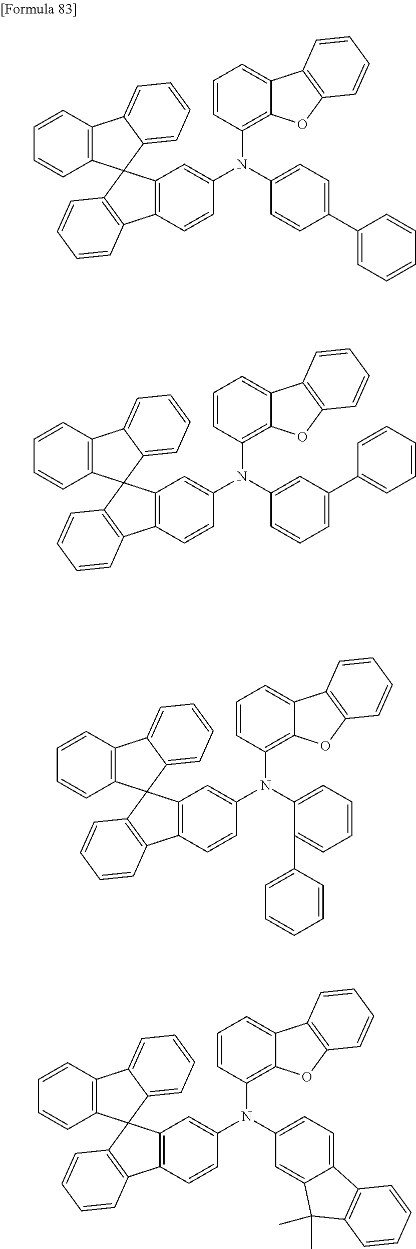 [SMILES: c1ccc(-c2ccc(N(c3ccc4c(c3)C3(c5ccccc5-c5ccccc53)c3ccccc3-4)c3cccc4c3oc3ccccc34)cc2)cc1 ; c1ccc(-c2cccc(N(c3ccc4c(c3)C3(c5ccccc5-c5ccccc53)c3ccccc3-4)c3cccc4c3oc3ccccc34)c2)cc1 ; c1ccc(-c2ccccc2N(c2ccc3c(c2)C2(c4ccccc4-c4ccccc42)c2ccccc2-3)c2cccc3c2oc2ccccc23)cc1 ; CC1(C)c2ccccc2-c2ccc(N(c3ccc4c(c3)C3(c5ccccc5-c5ccccc53)c3ccccc3-4)c3cccc4c3oc3ccccc34)cc21]

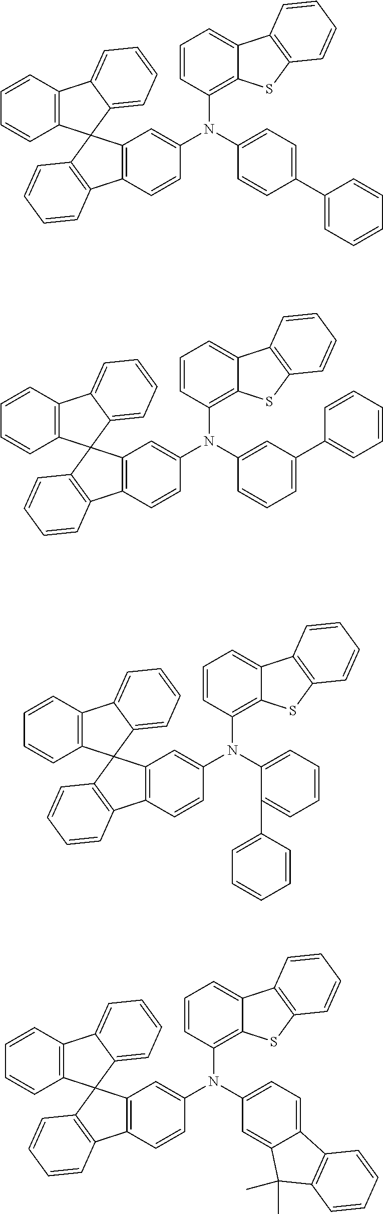 [SMILES: c1ccc(-c2ccc(N(c3ccc4c(c3)C3(c5ccccc5-c5ccccc53)c3ccccc3-4)c3cccc4c3sc3ccccc34)cc2)cc1 ; c1ccc(-c2cccc(N(c3ccc4c(c3)C3(c5ccccc5-c5ccccc53)c3ccccc3-4)c3cccc4c3sc3ccccc34)c2)cc1 ; c1ccc(-c2ccccc2N(c2ccc3c(c2)C2(c4ccccc4-c4ccccc42)c2ccccc2-3)c2cccc3c2sc2ccccc23)cc1 ; CC1(C)c2ccccc2-c2ccc(N(c3ccc4c(c3)C3(c5ccccc5-c5ccccc53)c3ccccc3-4)c3cccc4c3sc3ccccc34)cc21]

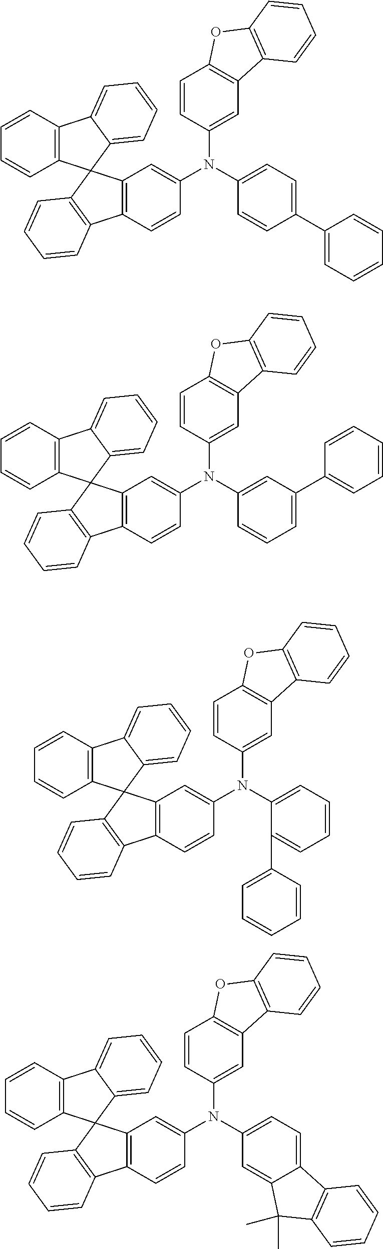

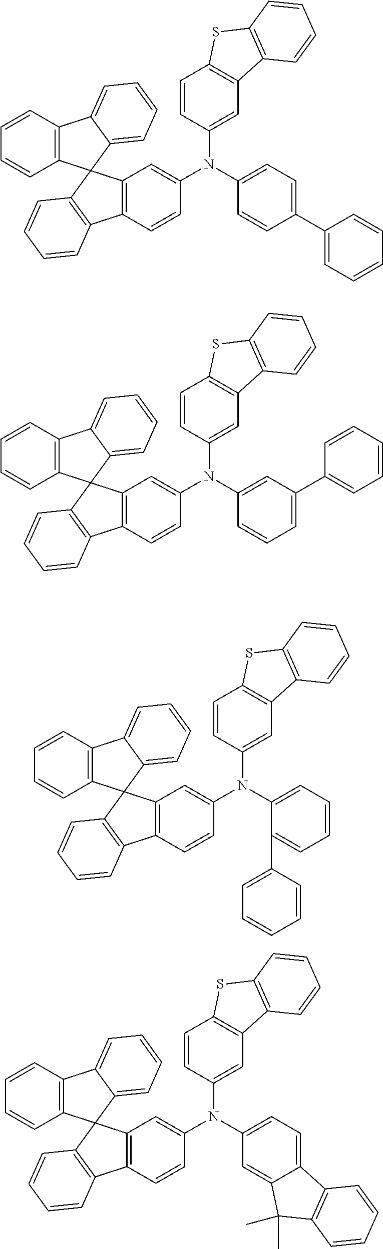

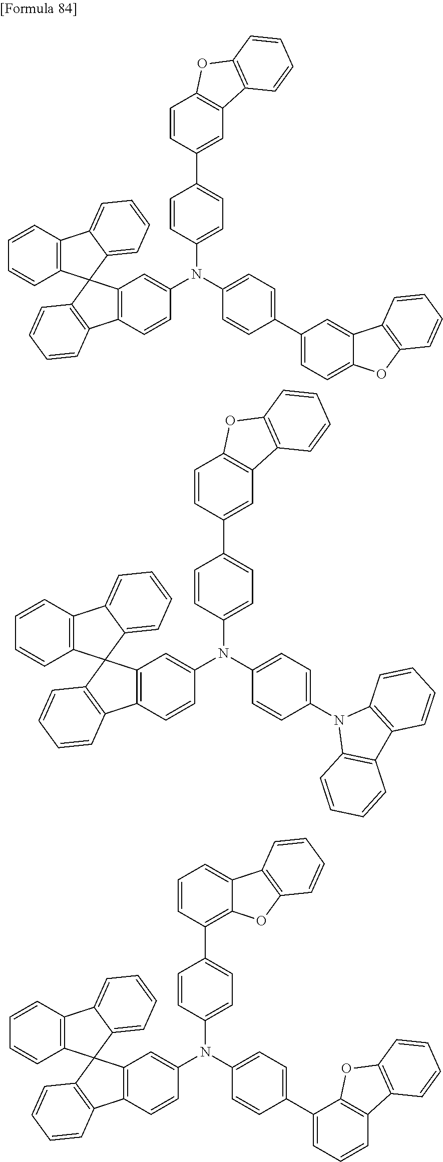

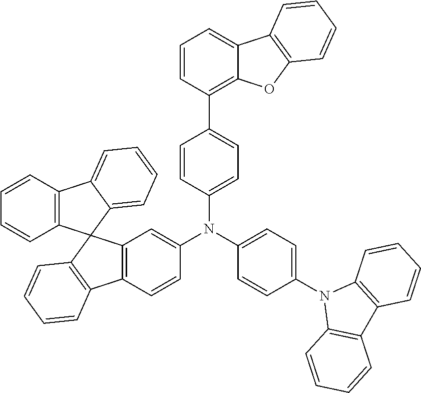 [SMILES: c1ccc2c(c1)-c1ccccc1C21c2ccccc2-c2ccc(N(c3ccc(-c4cccc5c4oc4ccccc45)cc3)c3ccc(-n4c5ccccc5c5ccccc54)cc3)cc21]

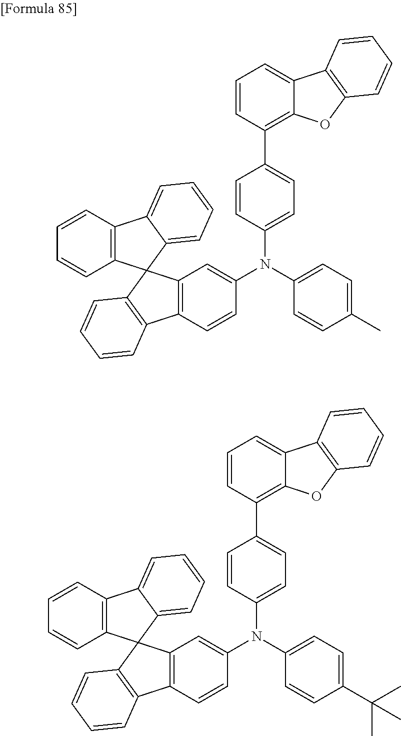 [SMILES: Cc1ccc(N(c2ccc(-c3cccc4c3oc3ccccc34)cc2)c2ccc3c(c2)C2(c4ccccc4-c4ccccc42)c2ccccc2-3)cc1 ; CC(C)(C)c1ccc(N(c2ccc(-c3cccc4c3oc3ccccc34)cc2)c2ccc3c(c2)C2(c4ccccc4-c4ccccc42)c2ccccc2-3)cc1]

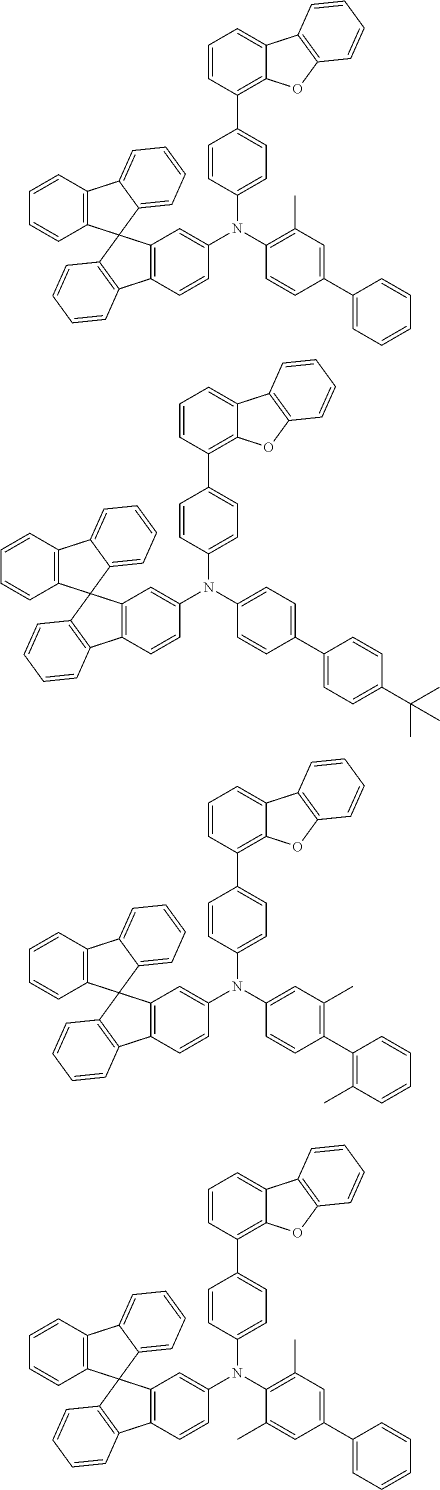 [SMILES: Cc1cc(-c2ccccc2)ccc1N(c1ccc(-c2cccc3c2oc2ccccc23)cc1)c1ccc2c(c1)C1(c3ccccc3-c3ccccc31)c1ccccc1-2 ; CC(C)(C)c1ccc(-c2ccc(N(c3ccc(-c4cccc5c4oc4ccccc45)cc3)c3ccc4c(c3)C3(c5ccccc5-c5ccccc53)c3ccccc3-4)cc2)cc1 ; Cc1ccccc1-c1ccc(N(c2ccc(-c3cccc4c3oc3ccccc34)cc2)c2ccc3c(c2)C2(c4ccccc4-c4ccccc42)c2ccccc2-3)cc1C ; Cc1cc(-c2ccccc2)cc(C)c1N(c1ccc(-c2cccc3c2oc2ccccc23)cc1)c1ccc2c(c1)C1(c3ccccc3-c3ccccc31)c1ccccc1-2]

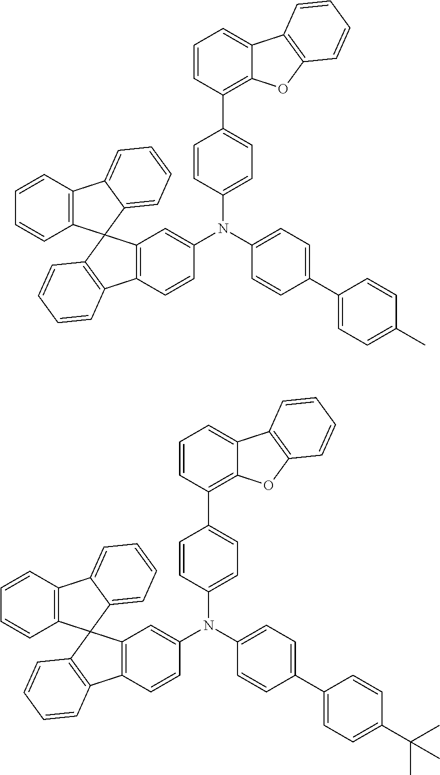 [SMILES: Cc1ccc(-c2ccc(N(c3ccc(-c4cccc5c4oc4ccccc45)cc3)c3ccc4c(c3)C3(c5ccccc5-c5ccccc53)c3ccccc3-4)cc2)cc1 ; CC(C)(C)c1ccc(-c2ccc(N(c3ccc(-c4cccc5c4oc4ccccc45)cc3)c3ccc4c(c3)C3(c5ccccc5-c5ccccc53)c3ccccc3-4)cc2)cc1]

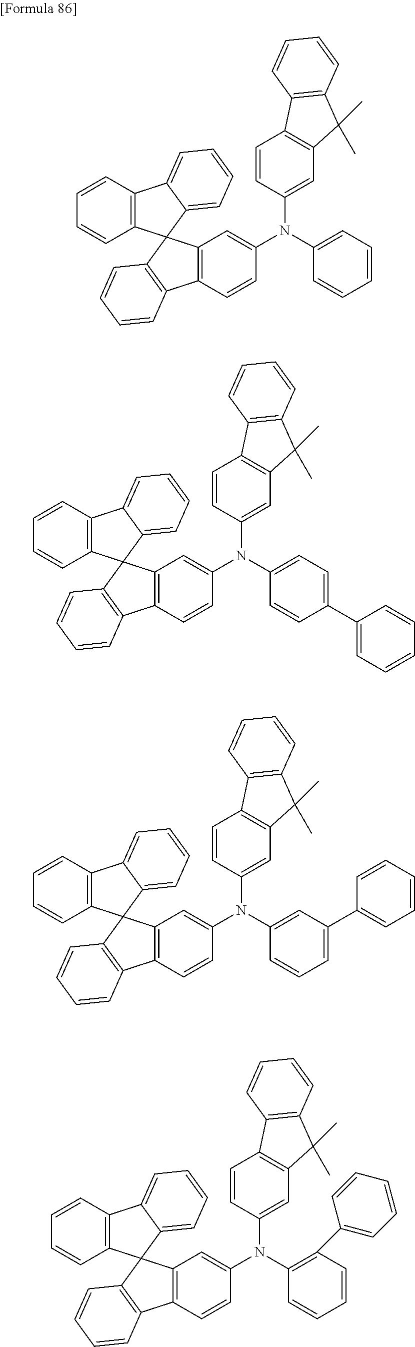 [SMILES: CC1(C)c2ccccc2-c2ccc(N(c3ccccc3)c3ccc4c(c3)C3(c5ccccc5-c5ccccc53)c3ccccc3-4)cc21 ; CC1(C)c2ccccc2-c2ccc(N(c3ccc(-c4ccccc4)cc3)c3ccc4c(c3)C3(c5ccccc5-c5ccccc53)c3ccccc3-4)cc21 ; CC1(C)c2ccccc2-c2ccc(N(c3cccc(-c4ccccc4)c3)c3ccc4c(c3)C3(c5ccccc5-c5ccccc53)c3ccccc3-4)cc21 ; CC1(C)c2ccccc2-c2ccc(N(c3ccc4c(c3)C3(c5ccccc5-c5ccccc53)c3ccccc3-4)c3ccccc3-c3ccccc3)cc21]

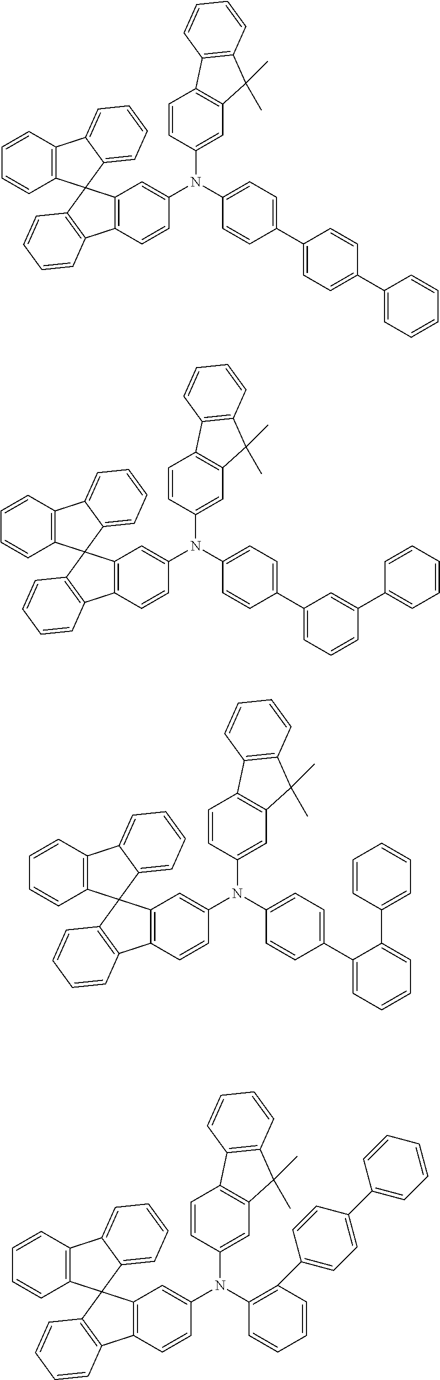 [SMILES: CC1(C)c2ccccc2-c2ccc(N(c3ccc(-c4ccc(-c5ccccc5)cc4)cc3)c3ccc4c(c3)C3(c5ccccc5-c5ccccc53)c3ccccc3-4)cc21 ; CC1(C)c2ccccc2-c2ccc(N(c3ccc(-c4cccc(-c5ccccc5)c4)cc3)c3ccc4c(c3)C3(c5ccccc5-c5ccccc53)c3ccccc3-4)cc21 ; CC1(C)c2ccccc2-c2ccc(N(c3ccc(-c4ccccc4-c4ccccc4)cc3)c3ccc4c(c3)C3(c5ccccc5-c5ccccc53)c3ccccc3-4)cc21 ; CC1(C)c2ccccc2-c2ccc(N(c3ccc4c(c3)C3(c5ccccc5-c5ccccc53)c3ccccc3-4)c3ccccc3-c3ccc(-c4ccccc4)cc3)cc21]

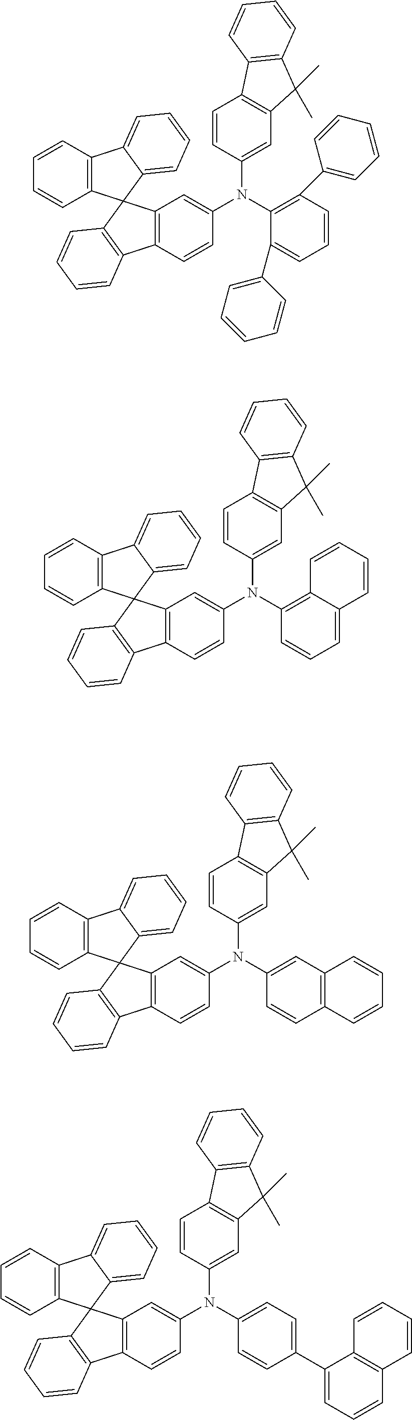 [SMILES: CC1(C)c2ccccc2-c2ccc(N(c3ccc4c(c3)C3(c5ccccc5-c5ccccc53)c3ccccc3-4)c3c(-c4ccccc4)cccc3-c3ccccc3)cc21 ; CC1(C)c2ccccc2-c2ccc(N(c3ccc4c(c3)C3(c5ccccc5-c5ccccc53)c3ccccc3-4)c3cccc4ccccc34)cc21 ; CC1(C)c2ccccc2-c2ccc(N(c3ccc4c(c3)C3(c5ccccc5-c5ccccc53)c3ccccc3-4)c3ccc4ccccc4c3)cc21 ; CC1(C)c2ccccc2-c2ccc(N(c3ccc(-c4cccc5ccccc45)cc3)c3ccc4c(c3)C3(c5ccccc5-c5ccccc53)c3ccccc3-4)cc21]

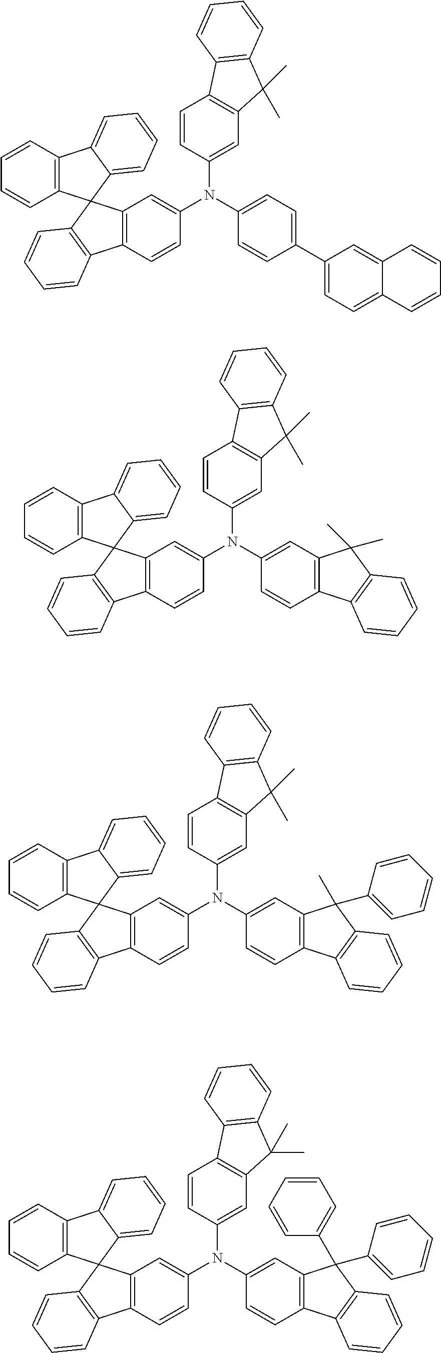 [SMILES: CC1(C)c2ccccc2-c2ccc(N(c3ccc(-c4ccc5ccccc5c4)cc3)c3ccc4c(c3)C3(c5ccccc5-c5ccccc53)c3ccccc3-4)cc21 ; CC1(C)c2ccccc2-c2ccc(N(c3ccc4c(c3)C(C)(C)c3ccccc3-4)c3ccc4c(c3)C3(c5ccccc5-c5ccccc53)c3ccccc3-4)cc21 ; CC1(C)c2ccccc2-c2ccc(N(c3ccc4c(c3)C(C)(c3ccccc3)c3ccccc3-4)c3ccc4c(c3)C3(c5ccccc5-c5ccccc53)c3ccccc3-4)cc21 ; CC1(C)c2ccccc2-c2ccc(N(c3ccc4c(c3)C(c3ccccc3)(c3ccccc3)c3ccccc3-4)c3ccc4c(c3)C3(c5ccccc5-c5ccccc53)c3ccccc3-4)cc21]

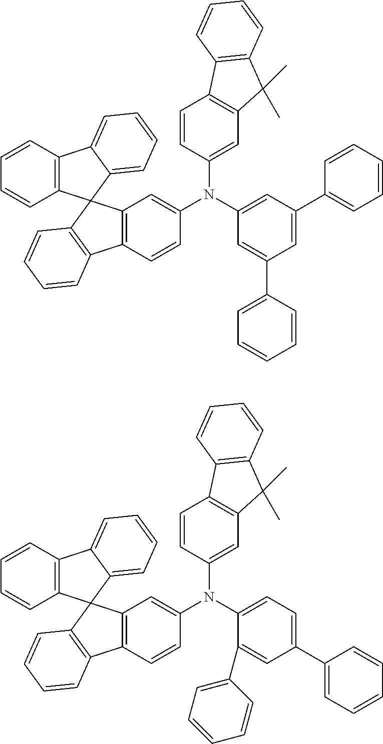 [SMILES: CC1(C)c2ccccc2-c2ccc(N(c3cc(-c4ccccc4)cc(-c4ccccc4)c3)c3ccc4c(c3)C3(c5ccccc5-c5ccccc53)c3ccccc3-4)cc21 ; CC1(C)c2ccccc2-c2ccc(N(c3ccc4c(c3)C3(c5ccccc5-c5ccccc53)c3ccccc3-4)c3ccc(-c4ccccc4)cc3-c3ccccc3)cc21]

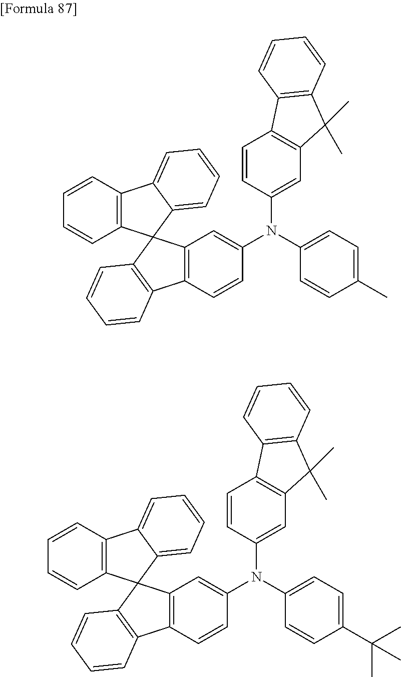 [SMILES: Cc1ccc(N(c2ccc3c(c2)C(C)(C)c2ccccc2-3)c2ccc3c(c2)C2(c4ccccc4-c4ccccc42)c2ccccc2-3)cc1 ; CC(C)(C)c1ccc(N(c2ccc3c(c2)C(C)(C)c2ccccc2-3)c2ccc3c(c2)C2(c4ccccc4-c4ccccc42)c2ccccc2-3)cc1]

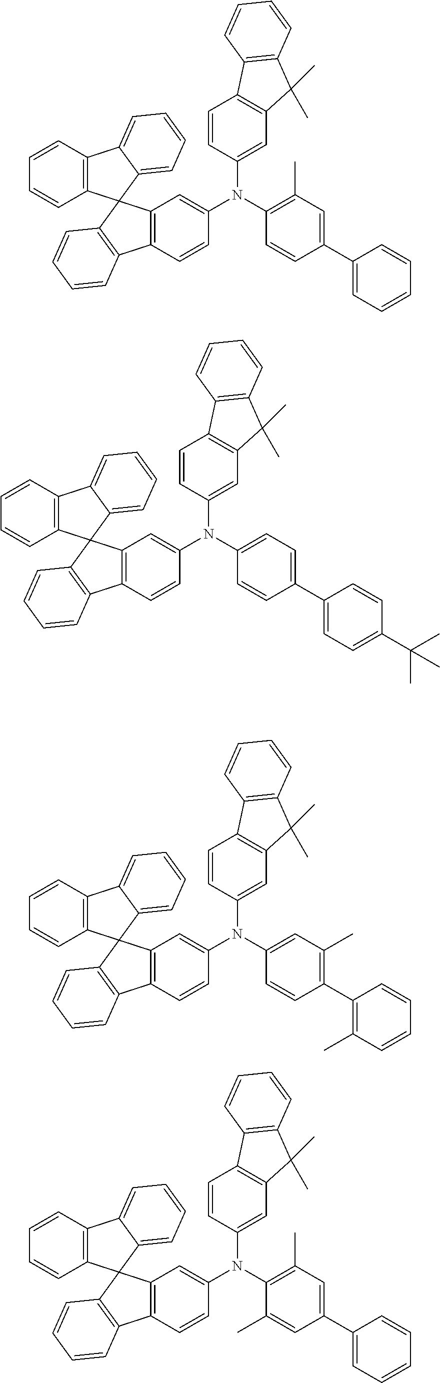 [SMILES: Cc1cc(-c2ccccc2)ccc1N(c1ccc2c(c1)C(C)(C)c1ccccc1-2)c1ccc2c(c1)C1(c3ccccc3-c3ccccc31)c1ccccc1-2 ; CC(C)(C)c1ccc(-c2ccc(N(c3ccc4c(c3)C(C)(C)c3ccccc3-4)c3ccc4c(c3)C3(c5ccccc5-c5ccccc53)c3ccccc3-4)cc2)cc1 ; Cc1ccccc1-c1ccc(N(c2ccc3c(c2)C(C)(C)c2ccccc2-3)c2ccc3c(c2)C2(c4ccccc4-c4ccccc42)c2ccccc2-3)cc1C ; Cc1cc(-c2ccccc2)cc(C)c1N(c1ccc2c(c1)C(C)(C)c1ccccc1-2)c1ccc2c(c1)C1(c3ccccc3-c3ccccc31)c1ccccc1-2]

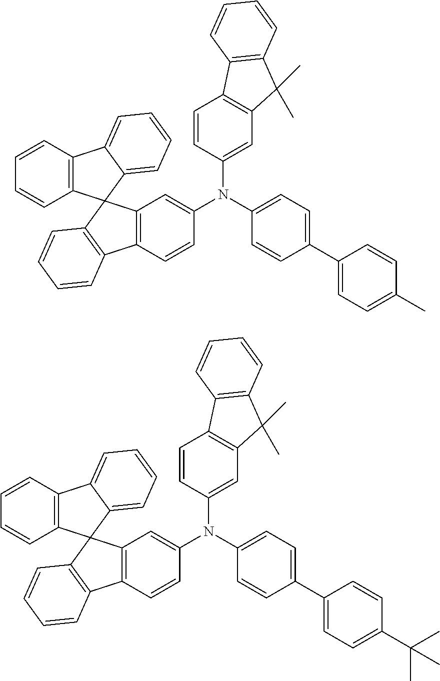 [SMILES: Cc1ccc(-c2ccc(N(c3ccc4c(c3)C(C)(C)c3ccccc3-4)c3ccc4c(c3)C3(c5ccccc5-c5ccccc53)c3ccccc3-4)cc2)cc1 ; CC(C)(C)c1ccc(-c2ccc(N(c3ccc4c(c3)C(C)(C)c3ccccc3-4)c3ccc4c(c3)C3(c5ccccc5-c5ccccc53)c3ccccc3-4)cc2)cc1]

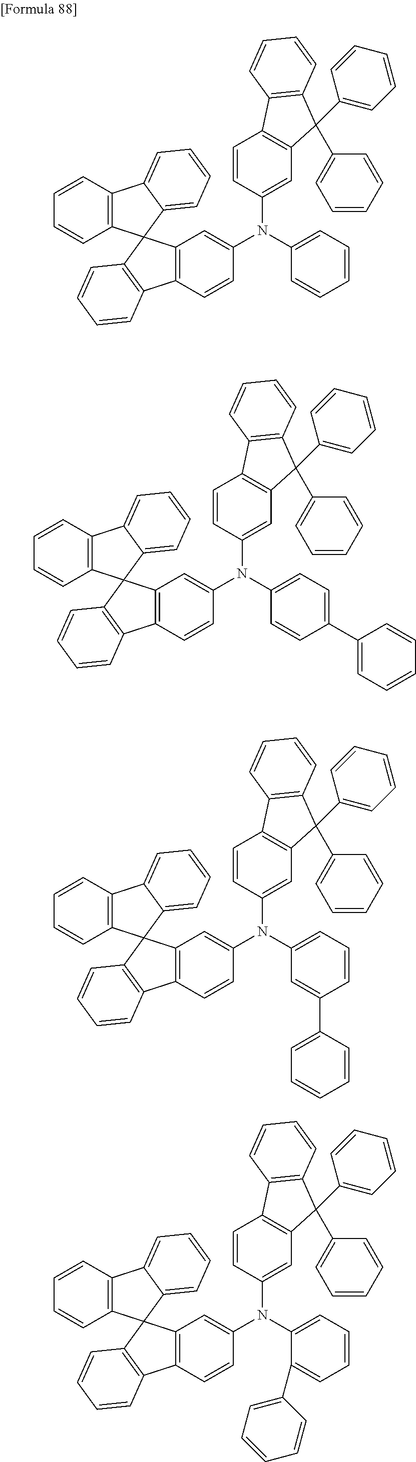

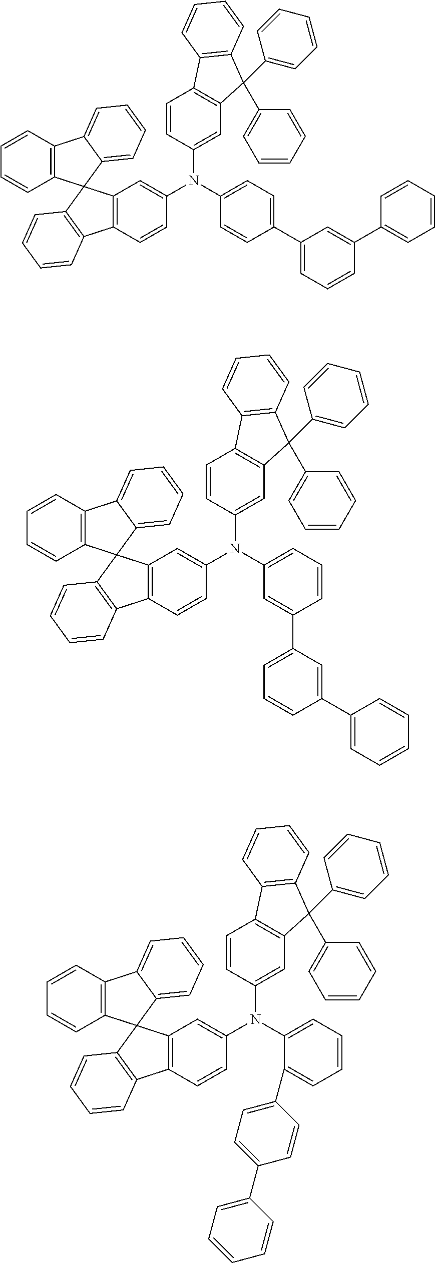 [SMILES: c1ccc(-c2cccc(-c3ccc(N(c4ccc5c(c4)C(c4ccccc4)(c4ccccc4)c4ccccc4-5)c4ccc5c(c4)C4(c6ccccc6-c6ccccc64)c4ccccc4-5)cc3)c2)cc1 ; c1ccc(-c2cccc(-c3cccc(N(c4ccc5c(c4)C(c4ccccc4)(c4ccccc4)c4ccccc4-5)c4ccc5c(c4)C4(c6ccccc6-c6ccccc64)c4ccccc4-5)c3)c2)cc1 ; c1ccc(-c2ccc(-c3ccccc3N(c3ccc4c(c3)C(c3ccccc3)(c3ccccc3)c3ccccc3-4)c3ccc4c(c3)C3(c5ccccc5-c5ccccc53)c3ccccc3-4)cc2)cc1]

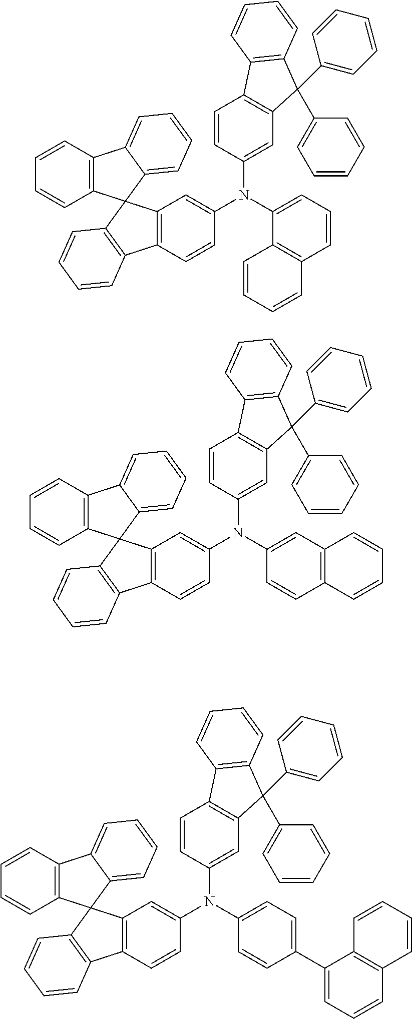

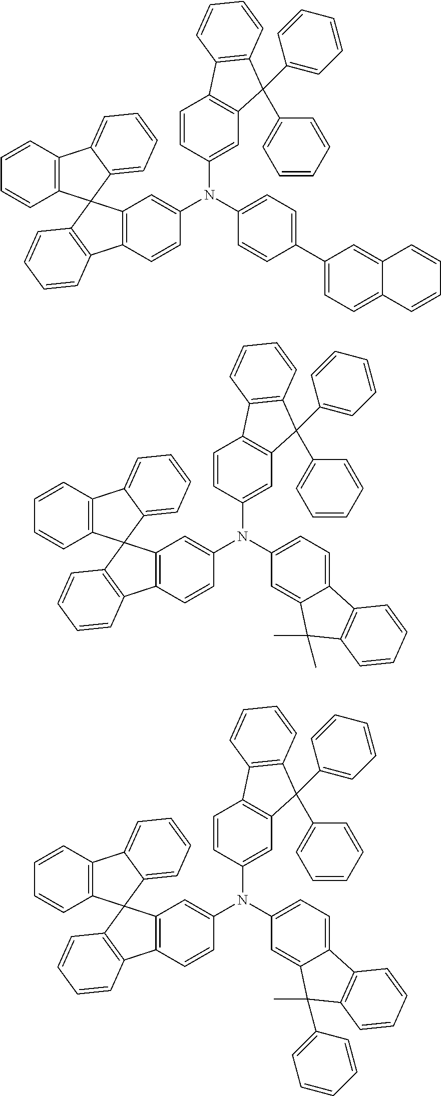

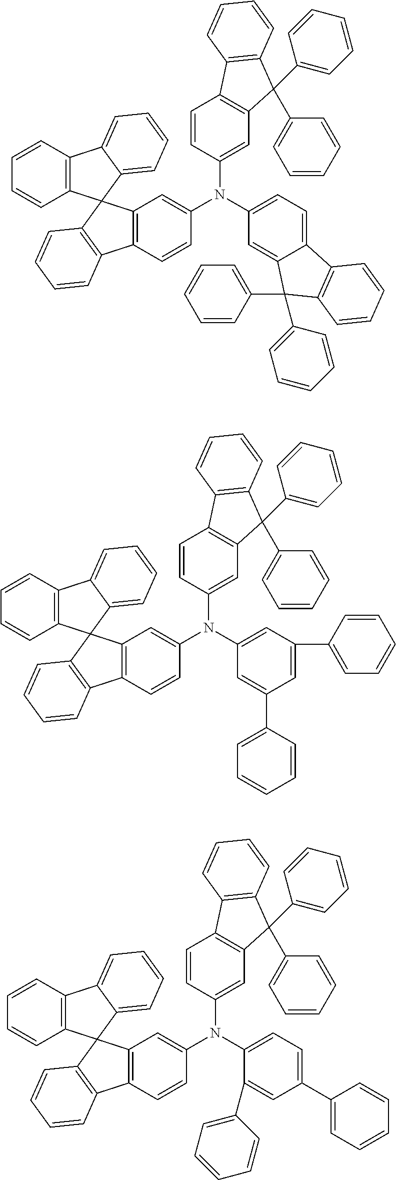 [SMILES: c1ccc(C2(c3ccccc3)c3ccccc3-c3ccc(N(c4ccc5c(c4)C(c4ccccc4)(c4ccccc4)c4ccccc4-5)c4ccc5c(c4)C4(c6ccccc6-c6ccccc64)c4ccccc4-5)cc32)cc1 ; c1ccc(-c2cc(-c3ccccc3)cc(N(c3ccc4c(c3)C(c3ccccc3)(c3ccccc3)c3ccccc3-4)c3ccc4c(c3)C3(c5ccccc5-c5ccccc53)c3ccccc3-4)c2)cc1 ; c1ccc(-c2ccc(N(c3ccc4c(c3)C(c3ccccc3)(c3ccccc3)c3ccccc3-4)c3ccc4c(c3)C3(c5ccccc5-c5ccccc53)c3ccccc3-4)c(-c3ccccc3)c2)cc1]

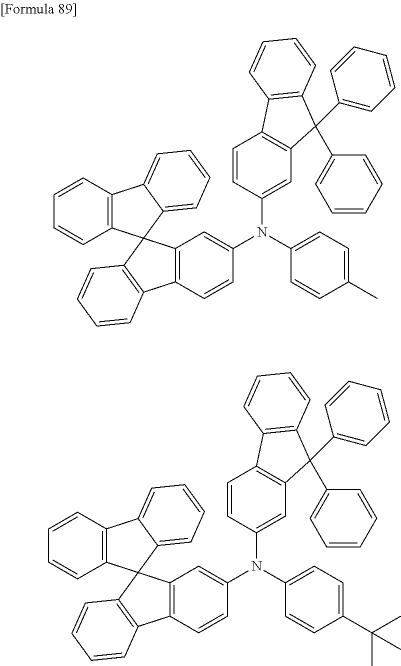 [SMILES: Cc1ccc(N(c2ccc3c(c2)C(c2ccccc2)(c2ccccc2)c2ccccc2-3)c2ccc3c(c2)C2(c4ccccc4-c4ccccc42)c2ccccc2-3)cc1 ; CC(C)(C)c1ccc(N(c2ccc3c(c2)C(c2ccccc2)(c2ccccc2)c2ccccc2-3)c2ccc3c(c2)C2(c4ccccc4-c4ccccc42)c2ccccc2-3)cc1]

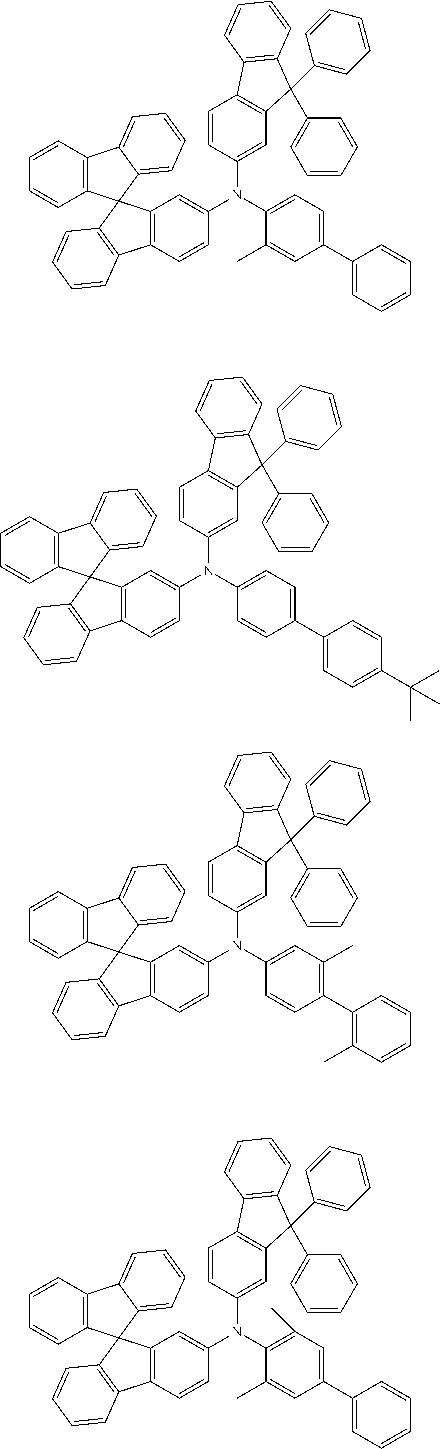 [SMILES: Cc1cc(-c2ccccc2)ccc1N(c1ccc2c(c1)C(c1ccccc1)(c1ccccc1)c1ccccc1-2)c1ccc2c(c1)C1(c3ccccc3-c3ccccc31)c1ccccc1-2 ; CC(C)(C)c1ccc(-c2ccc(N(c3ccc4c(c3)C(c3ccccc3)(c3ccccc3)c3ccccc3-4)c3ccc4c(c3)C3(c5ccccc5-c5ccccc53)c3ccccc3-4)cc2)cc1 ; Cc1ccccc1-c1ccc(N(c2ccc3c(c2)C(c2ccccc2)(c2ccccc2)c2ccccc2-3)c2ccc3c(c2)C2(c4ccccc4-c4ccccc42)c2ccccc2-3)cc1C ; Cc1cc(-c2ccccc2)cc(C)c1N(c1ccc2c(c1)C(c1ccccc1)(c1ccccc1)c1ccccc1-2)c1ccc2c(c1)C1(c3ccccc3-c3ccccc31)c1ccccc1-2]

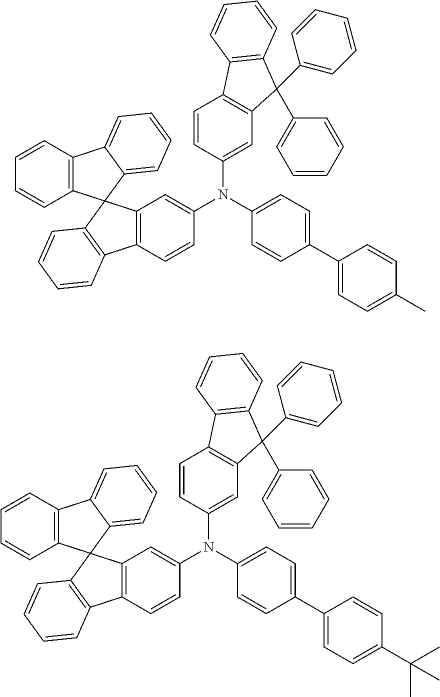 [SMILES: Cc1ccc(-c2ccc(N(c3ccc4c(c3)C(c3ccccc3)(c3ccccc3)c3ccccc3-4)c3ccc4c(c3)C3(c5ccccc5-c5ccccc53)c3ccccc3-4)cc2)cc1 ; CC(C)(C)c1ccc(-c2ccc(N(c3ccc4c(c3)C(c3ccccc3)(c3ccccc3)c3ccccc3-4)c3ccc4c(c3)C3(c5ccccc5-c5ccccc53)c3ccccc3-4)cc2)cc1]

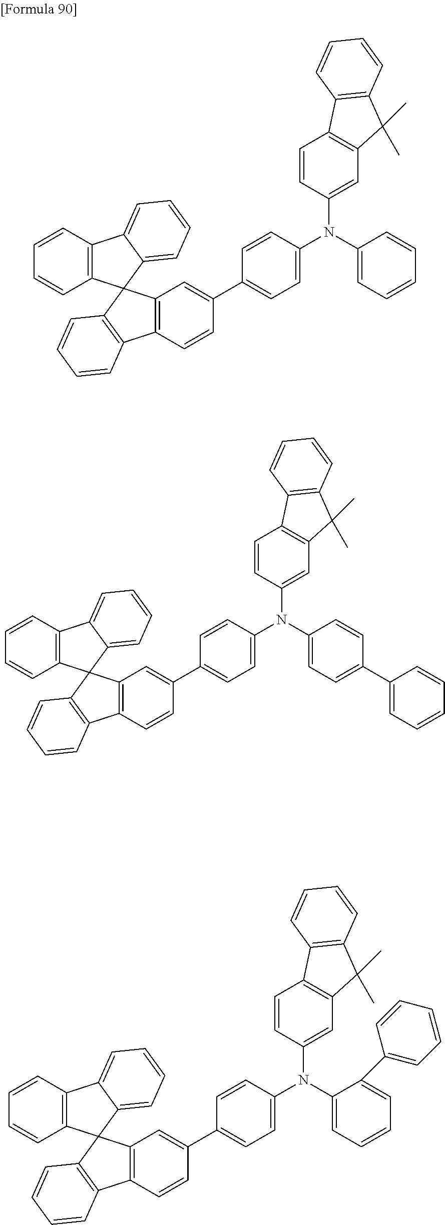 [SMILES: CC1(C)c2ccccc2-c2ccc(N(c3ccccc3)c3ccc(-c4ccc5c(c4)C4(c6ccccc6-c6ccccc64)c4ccccc4-5)cc3)cc21 ; CC1(C)c2ccccc2-c2ccc(N(c3ccc(-c4ccccc4)cc3)c3ccc(-c4ccc5c(c4)C4(c6ccccc6-c6ccccc64)c4ccccc4-5)cc3)cc21 ; CC1(C)c2ccccc2-c2ccc(N(c3ccc(-c4ccc5c(c4)C4(c6ccccc6-c6ccccc64)c4ccccc4-5)cc3)c3ccccc3-c3ccccc3)cc21]

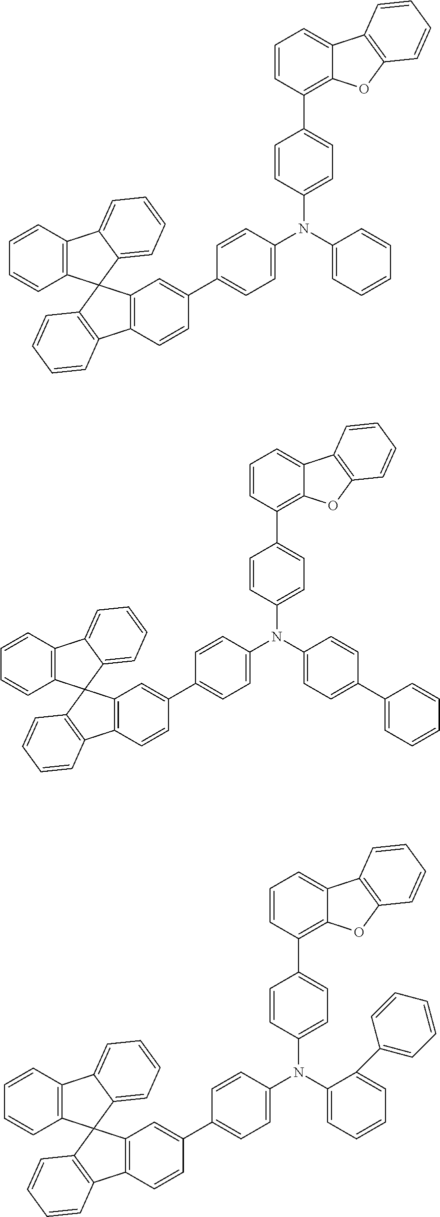 [SMILES: c1ccc(N(c2ccc(-c3ccc4c(c3)C3(c5ccccc5-c5ccccc53)c3ccccc3-4)cc2)c2ccc(-c3cccc4c3oc3ccccc34)cc2)cc1 ; c1ccc(-c2ccc(N(c3ccc(-c4ccc5c(c4)C4(c6ccccc6-c6ccccc64)c4ccccc4-5)cc3)c3ccc(-c4cccc5c4oc4ccccc45)cc3)cc2)cc1 ; c1ccc(-c2ccccc2N(c2ccc(-c3ccc4c(c3)C3(c5ccccc5-c5ccccc53)c3ccccc3-4)cc2)c2ccc(-c3cccc4c3oc3ccccc34)cc2)cc1]

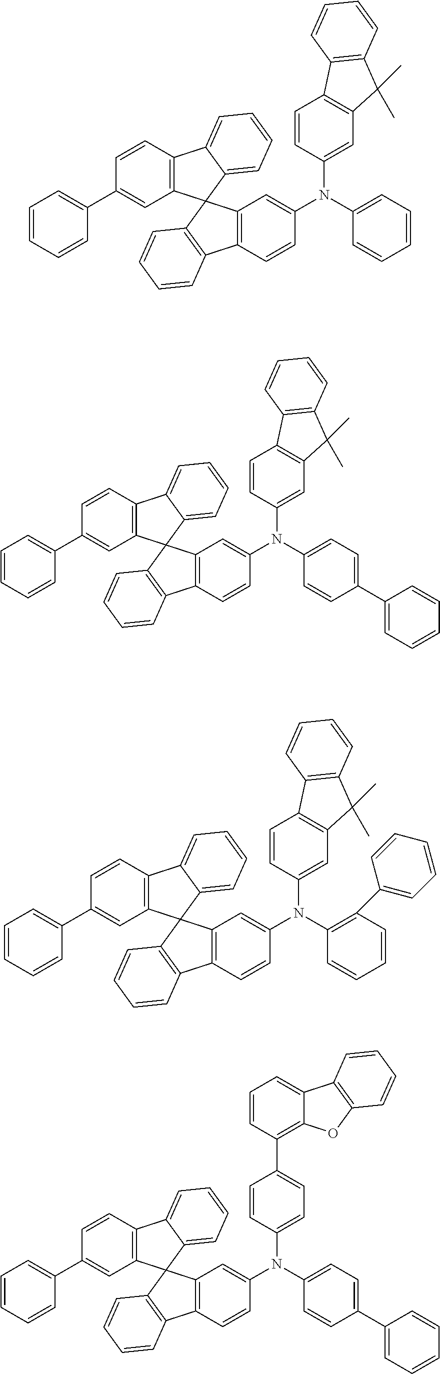 [SMILES: CC1(C)c2ccccc2-c2ccc(N(c3ccccc3)c3ccc4c(c3)C3(c5ccccc5-c5ccc(-c6ccccc6)cc53)c3ccccc3-4)cc21 ; CC1(C)c2ccccc2-c2ccc(N(c3ccc(-c4ccccc4)cc3)c3ccc4c(c3)C3(c5ccccc5-c5ccc(-c6ccccc6)cc53)c3ccccc3-4)cc21 ; CC1(C)c2ccccc2-c2ccc(N(c3ccc4c(c3)C3(c5ccccc5-c5ccc(-c6ccccc6)cc53)c3ccccc3-4)c3ccccc3-c3ccccc3)cc21 ; c1ccc(-c2ccc(N(c3ccc(-c4cccc5c4oc4ccccc45)cc3)c3ccc4c(c3)C3(c5ccccc5-c5ccc(-c6ccccc6)cc53)c3ccccc3-4)cc2)cc1]

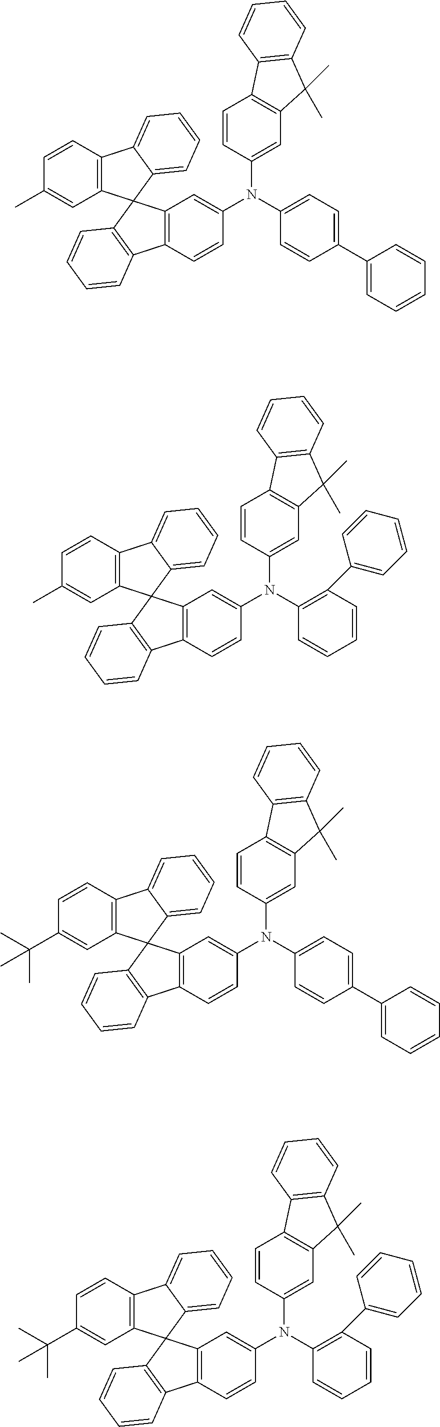 [SMILES: Cc1ccc2c(c1)C1(c3ccccc3-2)c2ccccc2-c2ccc(N(c3ccc(-c4ccccc4)cc3)c3ccc4c(c3)C(C)(C)c3ccccc3-4)cc21 ; Cc1ccc2c(c1)C1(c3ccccc3-2)c2ccccc2-c2ccc(N(c3ccc4c(c3)C(C)(C)c3ccccc3-4)c3ccccc3-c3ccccc3)cc21 ; CC(C)(C)c1ccc2c(c1)C1(c3ccccc3-c3ccc(N(c4ccc(-c5ccccc5)cc4)c4ccc5c(c4)C(C)(C)c4ccccc4-5)cc31)c1ccccc1-2 ; CC(C)(C)c1ccc2c(c1)C1(c3ccccc3-c3ccc(N(c4ccc5c(c4)C(C)(C)c4ccccc4-5)c4ccccc4-c4ccccc4)cc31)c1ccccc1-2]

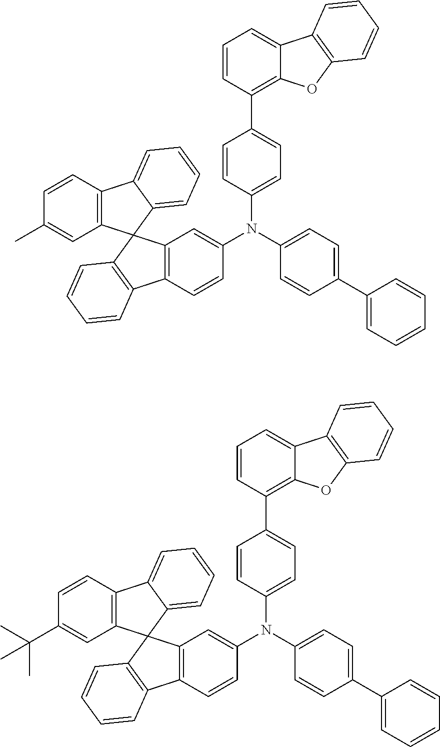 [SMILES: Cc1ccc2c(c1)C1(c3ccccc3-2)c2ccccc2-c2ccc(N(c3ccc(-c4ccccc4)cc3)c3ccc(-c4cccc5c4oc4ccccc45)cc3)cc21 ; CC(C)(C)c1ccc2c(c1)C1(c3ccccc3-c3ccc(N(c4ccc(-c5ccccc5)cc4)c4ccc(-c5cccc6c5oc5ccccc56)cc4)cc31)c1ccccc1-2]

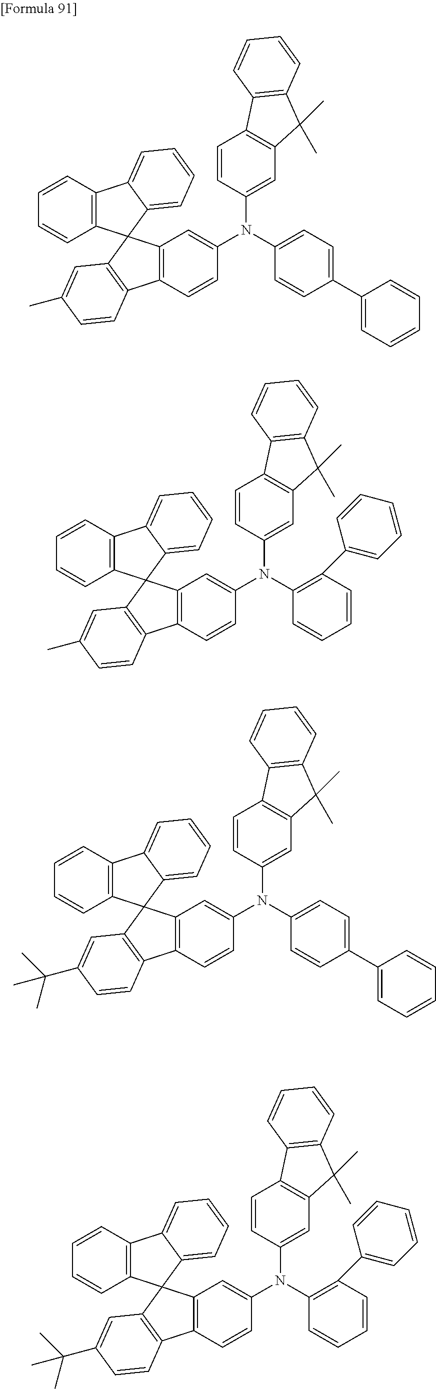 [SMILES: Cc1ccc2c(c1)C1(c3ccccc3-c3ccccc31)c1cc(N(c3ccc(-c4ccccc4)cc3)c3ccc4c(c3)C(C)(C)c3ccccc3-4)ccc1-2 ; Cc1ccc2c(c1)C1(c3ccccc3-c3ccccc31)c1cc(N(c3ccc4c(c3)C(C)(C)c3ccccc3-4)c3ccccc3-c3ccccc3)ccc1-2 ; CC(C)(C)c1ccc2c(c1)C1(c3ccccc3-c3ccccc31)c1cc(N(c3ccc(-c4ccccc4)cc3)c3ccc4c(c3)C(C)(C)c3ccccc3-4)ccc1-2 ; CC(C)(C)c1ccc2c(c1)C1(c3ccccc3-c3ccccc31)c1cc(N(c3ccc4c(c3)C(C)(C)c3ccccc3-4)c3ccccc3-c3ccccc3)ccc1-2]

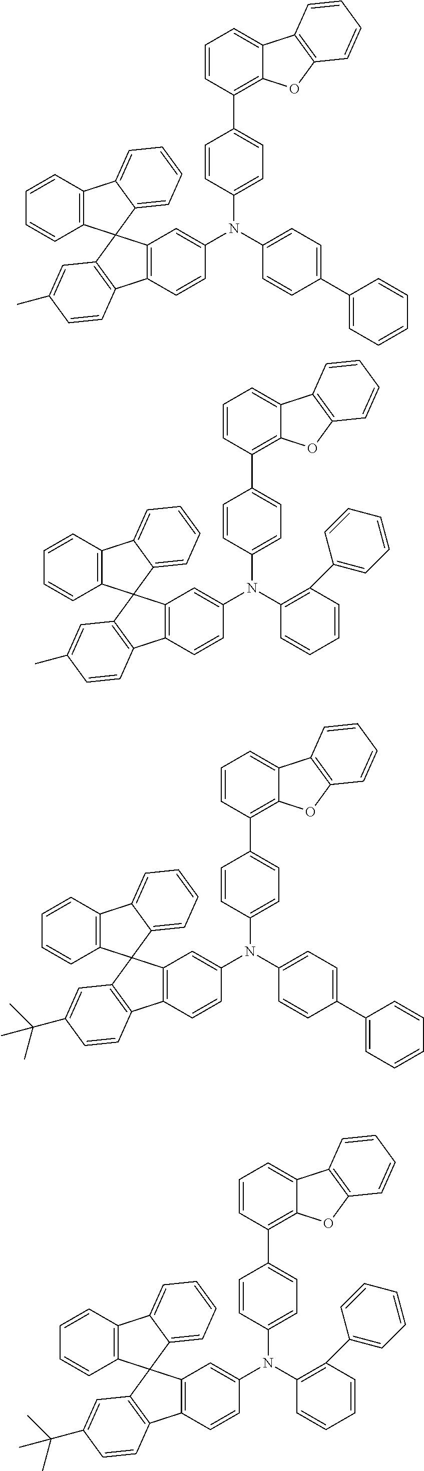

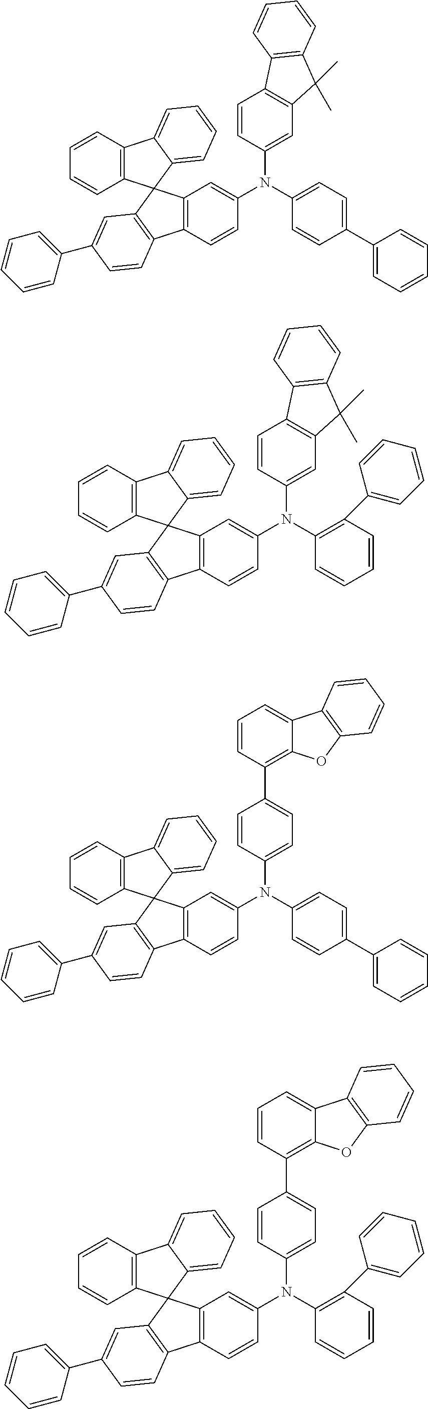 [SMILES: CC1(C)c2ccccc2-c2ccc(N(c3ccc(-c4ccccc4)cc3)c3ccc4c(c3)C3(c5ccccc5-c5ccccc53)c3cc(-c5ccccc5)ccc3-4)cc21 ; CC1(C)c2ccccc2-c2ccc(N(c3ccc4c(c3)C3(c5ccccc5-c5ccccc53)c3cc(-c5ccccc5)ccc3-4)c3ccccc3-c3ccccc3)cc21 ; c1ccc(-c2ccc(N(c3ccc(-c4cccc5c4oc4ccccc45)cc3)c3ccc4c(c3)C3(c5ccccc5-c5ccccc53)c3cc(-c5ccccc5)ccc3-4)cc2)cc1 ; c1ccc(-c2ccc3c(c2)C2(c4ccccc4-c4ccccc42)c2cc(N(c4ccc(-c5cccc6c5oc5ccccc56)cc4)c4ccccc4-c4ccccc4)ccc2-3)cc1]

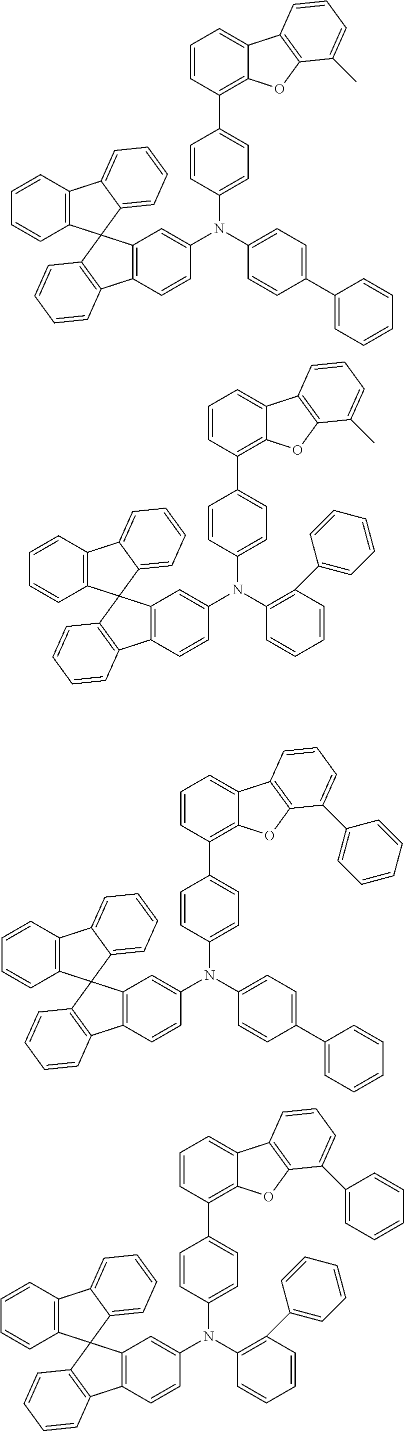

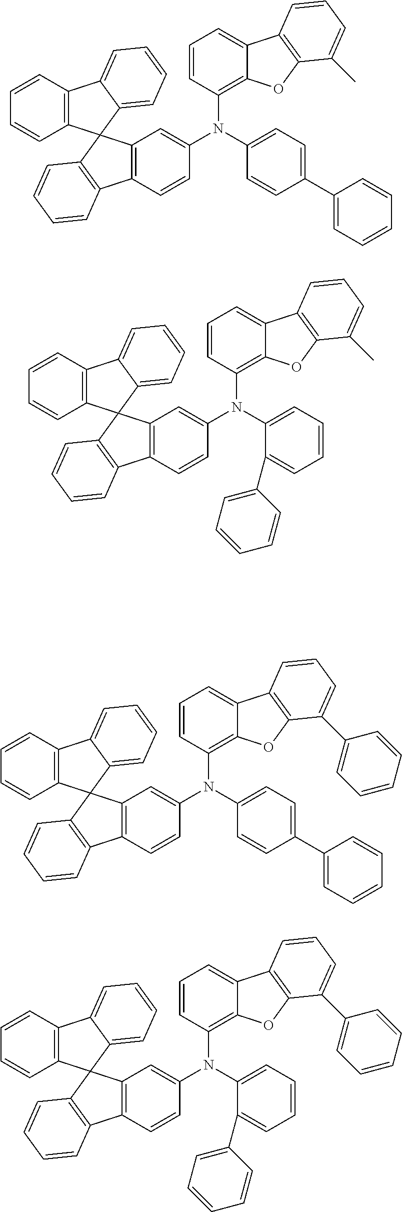

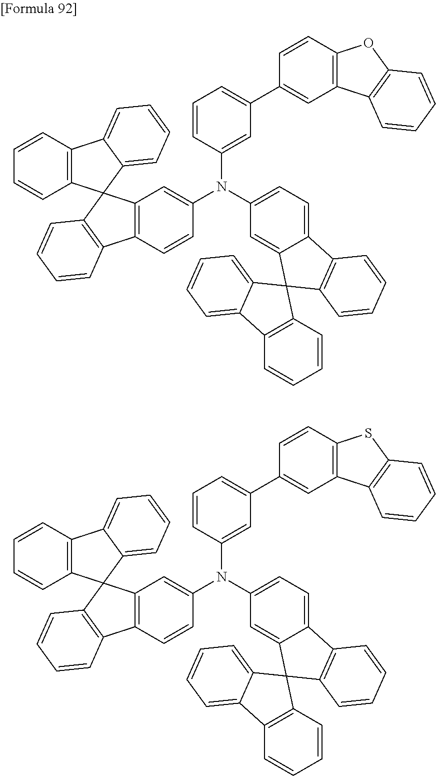

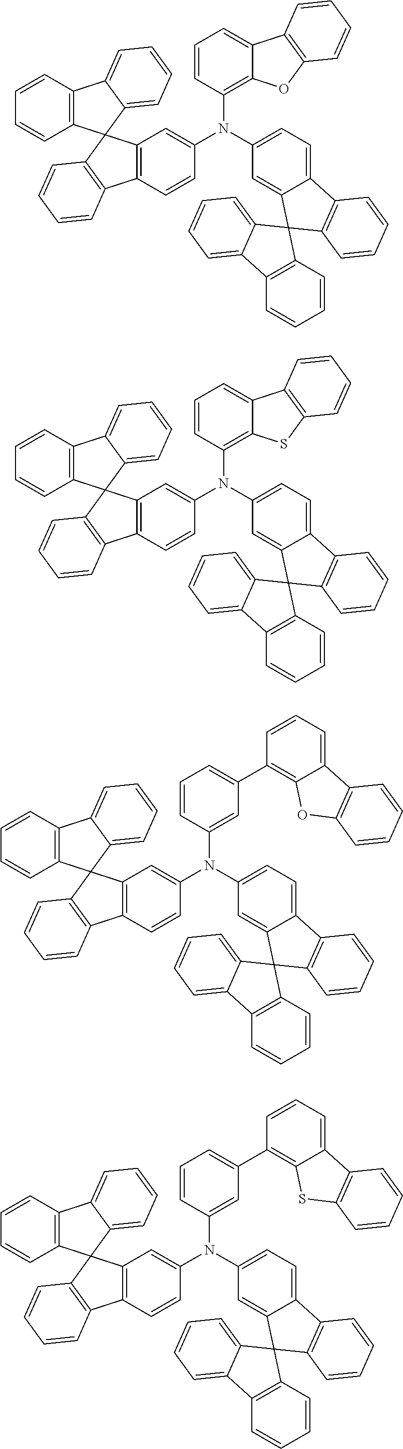

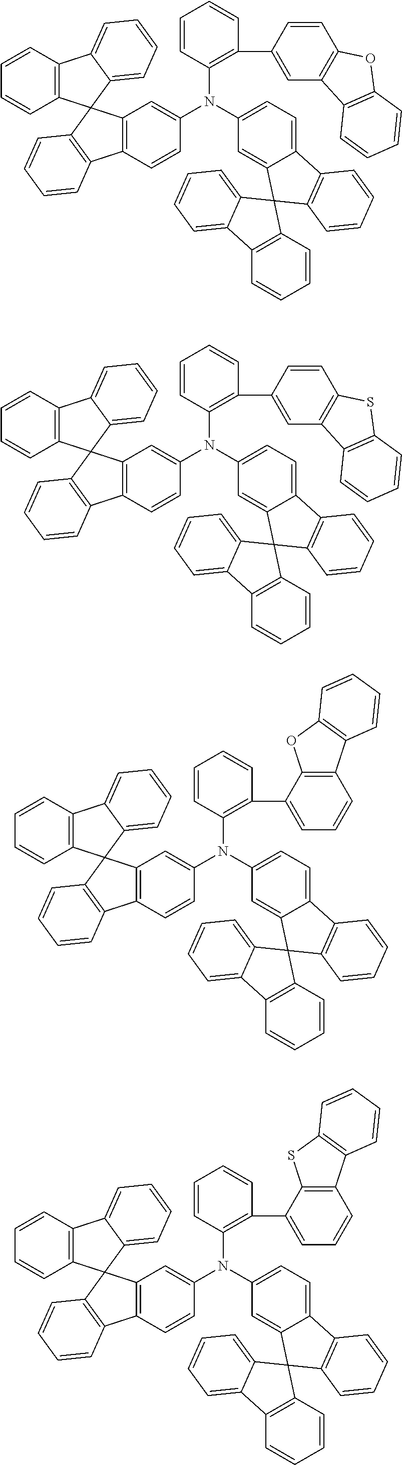 [SMILES: c1ccc(N(c2ccc3c(c2)C2(c4ccccc4-c4ccccc42)c2ccccc2-3)c2ccc3c(c2)C2(c4ccccc4-c4ccccc42)c2ccccc2-3)c(-c2ccc3oc4ccccc4c3c2)c1 ; c1ccc(N(c2ccc3c(c2)C2(c4ccccc4-c4ccccc42)c2ccccc2-3)c2ccc3c(c2)C2(c4ccccc4-c4ccccc42)c2ccccc2-3)c(-c2ccc3sc4ccccc4c3c2)c1 ; c1ccc(N(c2ccc3c(c2)C2(c4ccccc4-c4ccccc42)c2ccccc2-3)c2ccc3c(c2)C2(c4ccccc4-c4ccccc42)c2ccccc2-3)c(-c2cccc3c2oc2ccccc23)c1 ; c1ccc(N(c2ccc3c(c2)C2(c4ccccc4-c4ccccc42)c2ccccc2-3)c2ccc3c(c2)C2(c4ccccc4-c4ccccc42)c2ccccc2-3)c(-c2cccc3c2sc2ccccc23)c1]

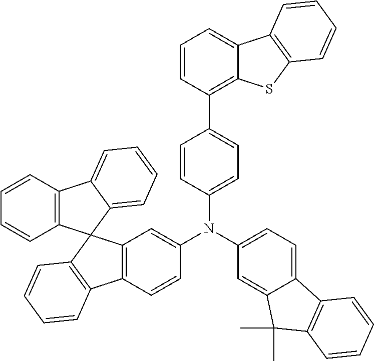 [SMILES: CC1(C)c2ccccc2-c2ccc(N(c3ccc(-c4cccc5c4sc4ccccc45)cc3)c3ccc4c(c3)C3(c5ccccc5-c5ccccc53)c3ccccc3-4)cc21]

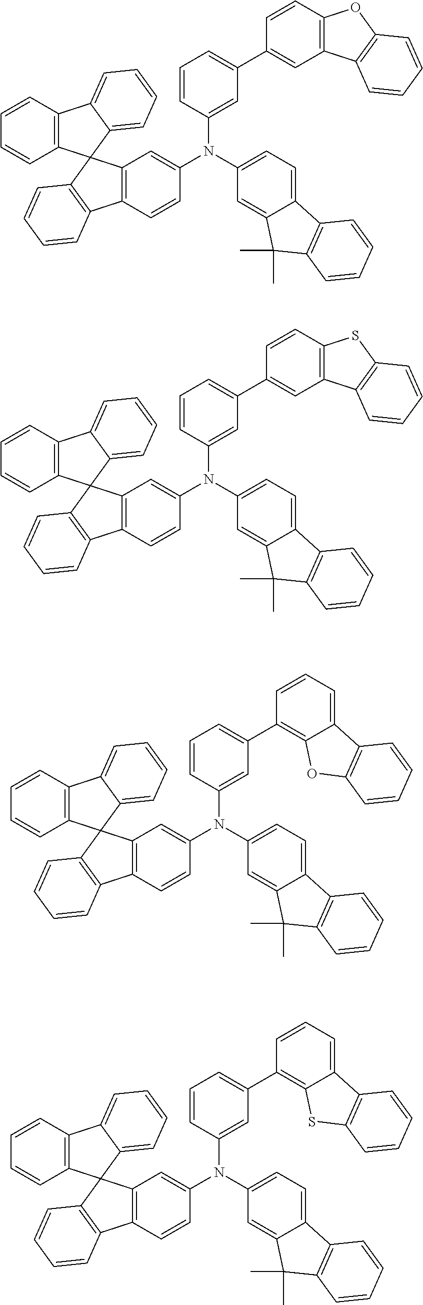 [SMILES: CC1(C)c2ccccc2-c2ccc(N(c3cccc(-c4ccc5oc6ccccc6c5c4)c3)c3ccc4c(c3)C3(c5ccccc5-c5ccccc53)c3ccccc3-4)cc21 ; CC1(C)c2ccccc2-c2ccc(N(c3cccc(-c4ccc5sc6ccccc6c5c4)c3)c3ccc4c(c3)C3(c5ccccc5-c5ccccc53)c3ccccc3-4)cc21 ; CC1(C)c2ccccc2-c2ccc(N(c3cccc(-c4cccc5c4oc4ccccc45)c3)c3ccc4c(c3)C3(c5ccccc5-c5ccccc53)c3ccccc3-4)cc21 ; CC1(C)c2ccccc2-c2ccc(N(c3cccc(-c4cccc5c4sc4ccccc45)c3)c3ccc4c(c3)C3(c5ccccc5-c5ccccc53)c3ccccc3-4)cc21]

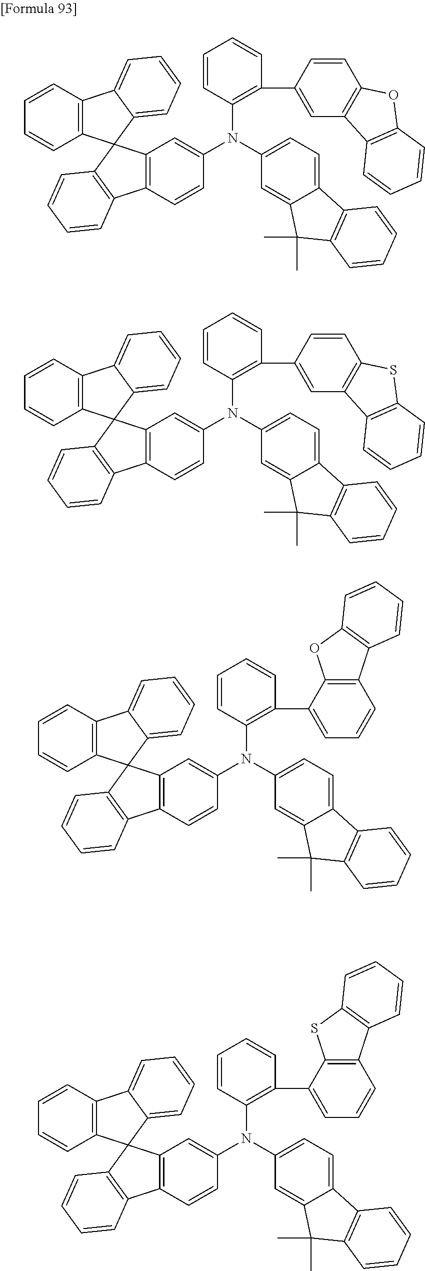

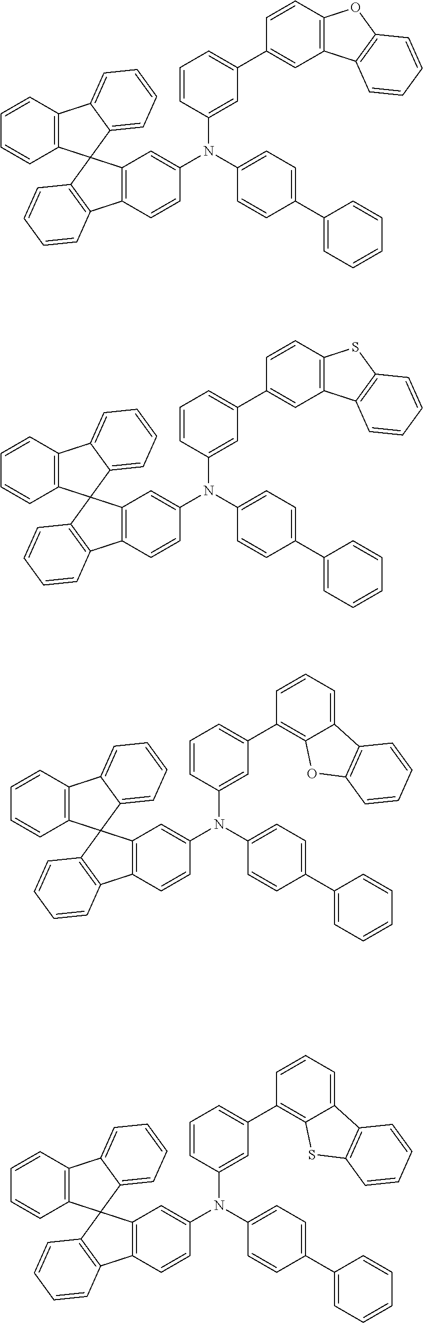 [SMILES: c1ccc(-c2ccc(N(c3cccc(-c4ccc5oc6ccccc6c5c4)c3)c3ccc4c(c3)C3(c5ccccc5-c5ccccc53)c3ccccc3-4)cc2)cc1 ; c1ccc(-c2ccc(N(c3cccc(-c4ccc5sc6ccccc6c5c4)c3)c3ccc4c(c3)C3(c5ccccc5-c5ccccc53)c3ccccc3-4)cc2)cc1 ; c1ccc(-c2ccc(N(c3cccc(-c4cccc5c4oc4ccccc45)c3)c3ccc4c(c3)C3(c5ccccc5-c5ccccc53)c3ccccc3-4)cc2)cc1 ; c1ccc(-c2ccc(N(c3cccc(-c4cccc5c4sc4ccccc45)c3)c3ccc4c(c3)C3(c5ccccc5-c5ccccc53)c3ccccc3-4)cc2)cc1]

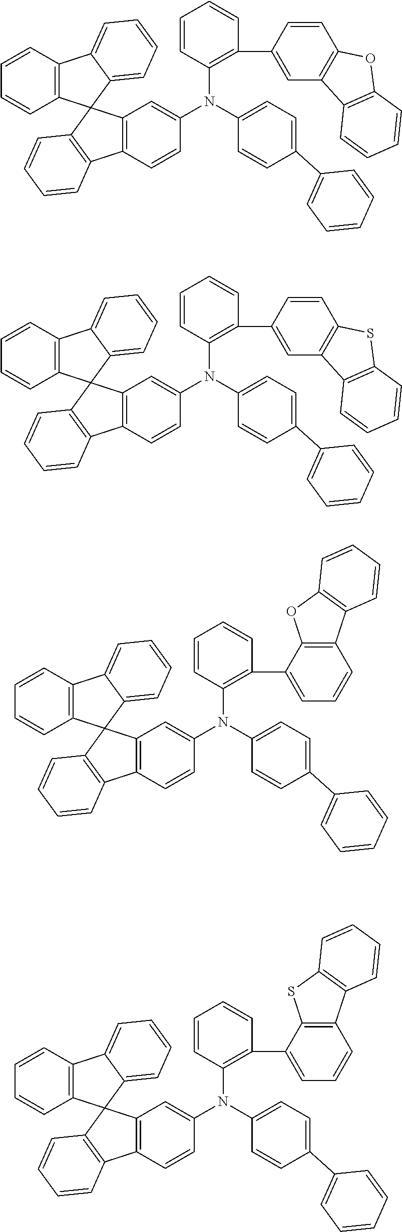

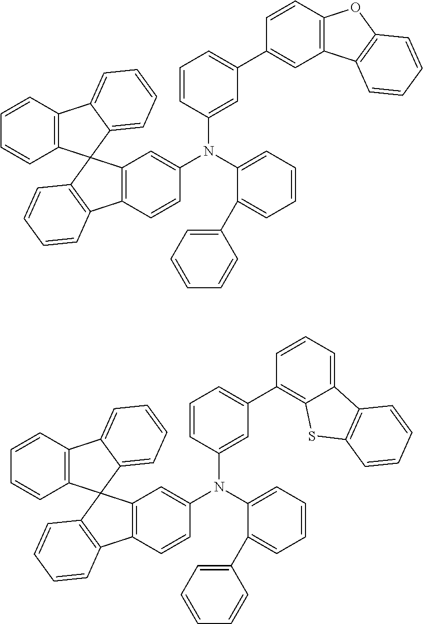 [SMILES: c1ccc(-c2ccccc2N(c2cccc(-c3ccc4oc5ccccc5c4c3)c2)c2ccc3c(c2)C2(c4ccccc4-c4ccccc42)c2ccccc2-3)cc1 ; c1ccc(-c2ccccc2N(c2cccc(-c3cccc4c3sc3ccccc34)c2)c2ccc3c(c2)C2(c4ccccc4-c4ccccc42)c2ccccc2-3)cc1]

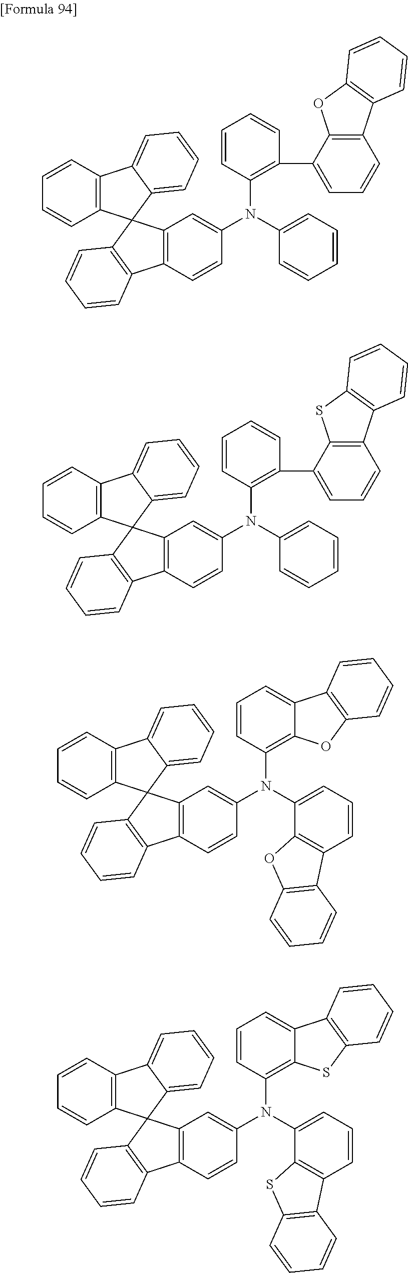 [SMILES: c1ccc(N(c2ccc3c(c2)C2(c4ccccc4-c4ccccc42)c2ccccc2-3)c2ccccc2-c2cccc3c2oc2ccccc23)cc1 ; c1ccc(N(c2ccc3c(c2)C2(c4ccccc4-c4ccccc42)c2ccccc2-3)c2ccccc2-c2cccc3c2sc2ccccc23)cc1 ; c1ccc2c(c1)-c1ccccc1C21c2ccccc2-c2ccc(N(c3cccc4c3oc3ccccc34)c3cccc4c3oc3ccccc34)cc21 ; c1ccc2c(c1)-c1ccccc1C21c2ccccc2-c2ccc(N(c3cccc4c3sc3ccccc34)c3cccc4c3sc3ccccc34)cc21]

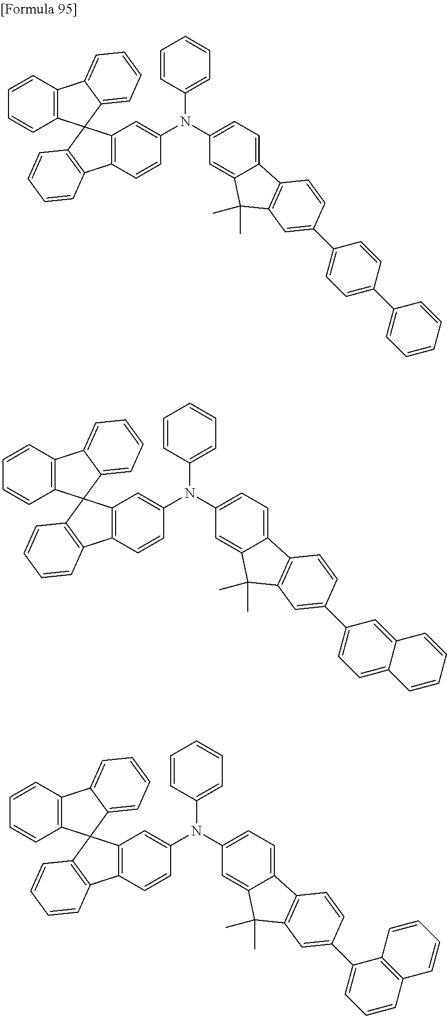 [SMILES: CC1(C)c2cc(-c3ccc(-c4ccccc4)cc3)ccc2-c2ccc(N(c3ccccc3)c3ccc4c(c3)C3(c5ccccc5-c5ccccc53)c3ccccc3-4)cc21 ; CC1(C)c2cc(-c3ccc4ccccc4c3)ccc2-c2ccc(N(c3ccccc3)c3ccc4c(c3)C3(c5ccccc5-c5ccccc53)c3ccccc3-4)cc21 ; CC1(C)c2cc(-c3cccc4ccccc34)ccc2-c2ccc(N(c3ccccc3)c3ccc4c(c3)C3(c5ccccc5-c5ccccc53)c3ccccc3-4)cc21]

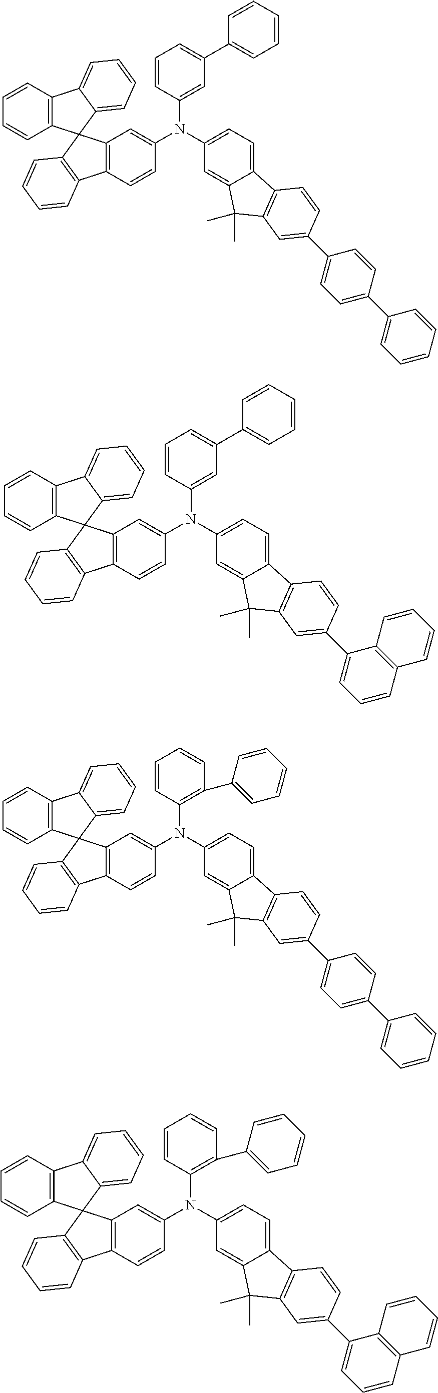 [SMILES: CC1(C)c2cc(-c3ccc(-c4ccccc4)cc3)ccc2-c2ccc(N(c3cccc(-c4ccccc4)c3)c3ccc4c(c3)C3(c5ccccc5-c5ccccc53)c3ccccc3-4)cc21 ; CC1(C)c2cc(-c3cccc4ccccc34)ccc2-c2ccc(N(c3cccc(-c4ccccc4)c3)c3ccc4c(c3)C3(c5ccccc5-c5ccccc53)c3ccccc3-4)cc21 ; CC1(C)c2cc(-c3ccc(-c4ccccc4)cc3)ccc2-c2ccc(N(c3ccc4c(c3)C3(c5ccccc5-c5ccccc53)c3ccccc3-4)c3ccccc3-c3ccccc3)cc21 ; CC1(C)c2cc(-c3cccc4ccccc34)ccc2-c2ccc(N(c3ccc4c(c3)C3(c5ccccc5-c5ccccc53)c3ccccc3-4)c3ccccc3-c3ccccc3)cc21]

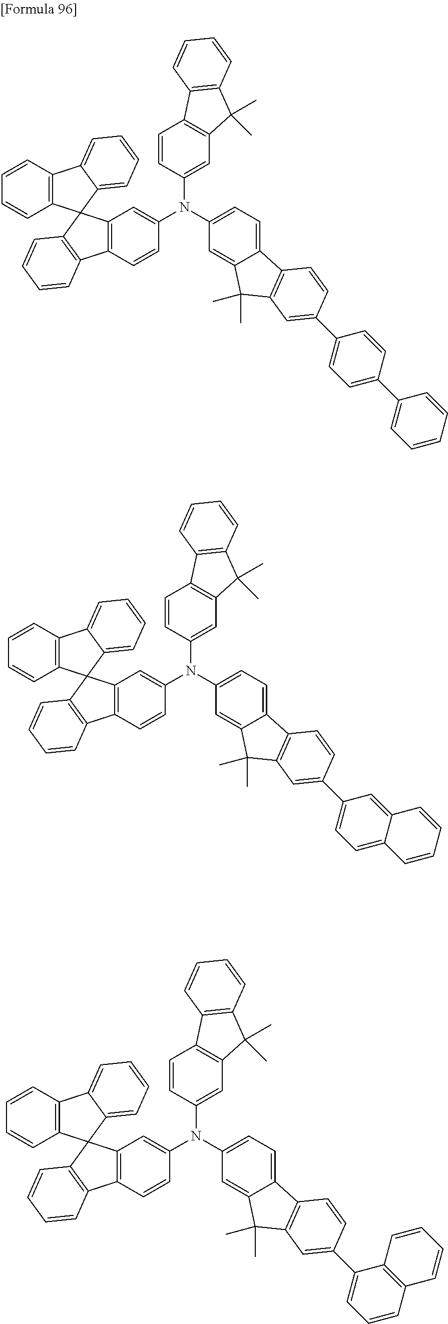 [SMILES: CC1(C)c2ccccc2-c2ccc(N(c3ccc4c(c3)C(C)(C)c3cc(-c5ccc(-c6ccccc6)cc5)ccc3-4)c3ccc4c(c3)C3(c5ccccc5-c5ccccc53)c3ccccc3-4)cc21 ; CC1(C)c2ccccc2-c2ccc(N(c3ccc4c(c3)C(C)(C)c3cc(-c5ccc6ccccc6c5)ccc3-4)c3ccc4c(c3)C3(c5ccccc5-c5ccccc53)c3ccccc3-4)cc21 ; CC1(C)c2ccccc2-c2ccc(N(c3ccc4c(c3)C(C)(C)c3cc(-c5cccc6ccccc56)ccc3-4)c3ccc4c(c3)C3(c5ccccc5-c5ccccc53)c3ccccc3-4)cc21]

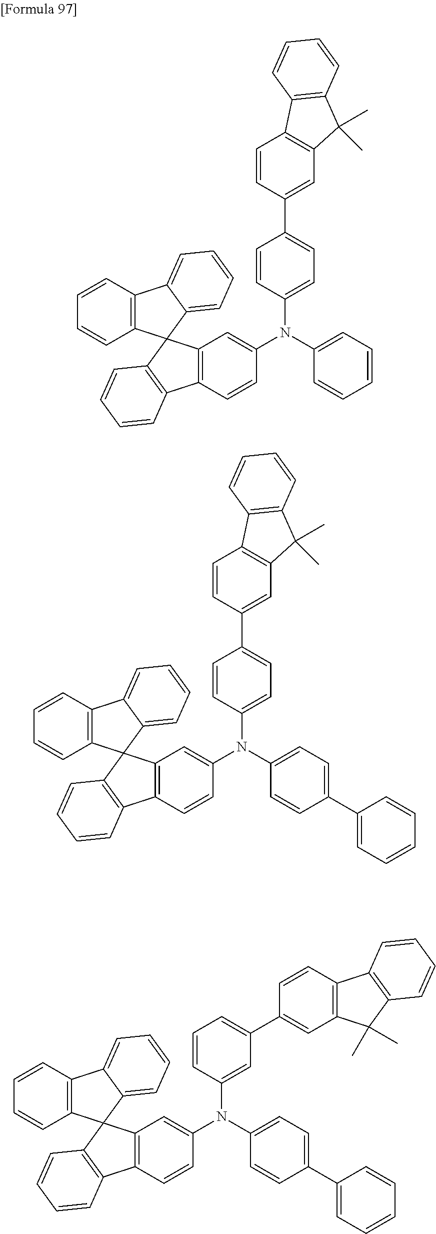 [SMILES: CC1(C)c2ccccc2-c2ccc(-c3ccc(N(c4ccccc4)c4ccc5c(c4)C4(c6ccccc6-c6ccccc64)c4ccccc4-5)cc3)cc21 ; CC1(C)c2ccccc2-c2ccc(-c3ccc(N(c4ccc(-c5ccccc5)cc4)c4ccc5c(c4)C4(c6ccccc6-c6ccccc64)c4ccccc4-5)cc3)cc21 ; CC1(C)c2ccccc2-c2ccc(-c3cccc(N(c4ccc(-c5ccccc5)cc4)c4ccc5c(c4)C4(c6ccccc6-c6ccccc64)c4ccccc4-5)c3)cc21]

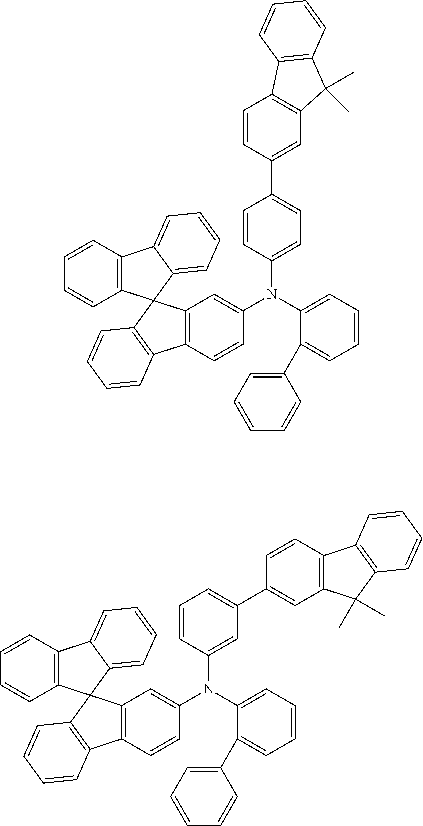 [SMILES: CC1(C)c2ccccc2-c2ccc(-c3ccc(N(c4ccc5c(c4)C4(c6ccccc6-c6ccccc64)c4ccccc4-5)c4ccccc4-c4ccccc4)cc3)cc21 ; CC1(C)c2ccccc2-c2ccc(-c3cccc(N(c4ccc5c(c4)C4(c6ccccc6-c6ccccc64)c4ccccc4-5)c4ccccc4-c4ccccc4)c3)cc21]

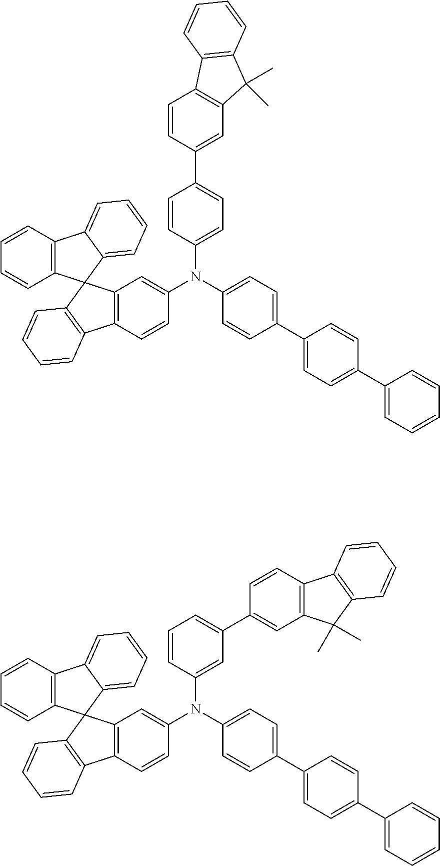 [SMILES: CC1(C)c2ccccc2-c2ccc(-c3ccc(N(c4ccc(-c5ccc(-c6ccccc6)cc5)cc4)c4ccc5c(c4)C4(c6ccccc6-c6ccccc64)c4ccccc4-5)cc3)cc21 ; CC1(C)c2ccccc2-c2ccc(-c3cccc(N(c4ccc(-c5ccc(-c6ccccc6)cc5)cc4)c4ccc5c(c4)C4(c6ccccc6-c6ccccc64)c4ccccc4-5)c3)cc21]

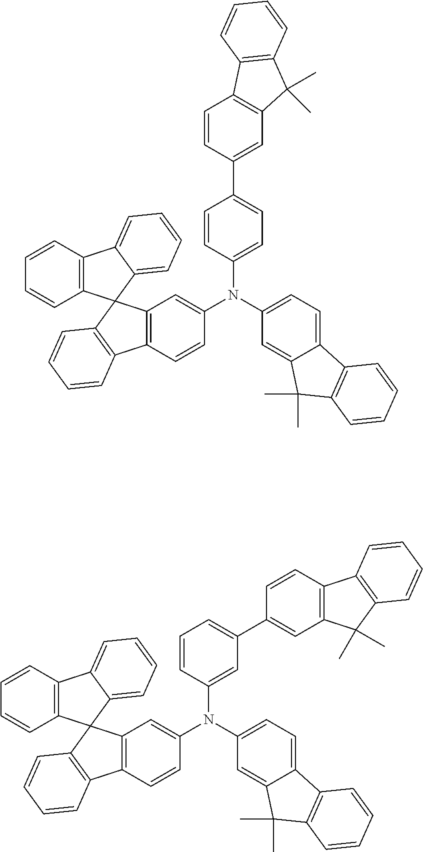 [SMILES: CC1(C)c2ccccc2-c2ccc(-c3ccc(N(c4ccc5c(c4)C(C)(C)c4ccccc4-5)c4ccc5c(c4)C4(c6ccccc6-c6ccccc64)c4ccccc4-5)cc3)cc21 ; CC1(C)c2ccccc2-c2ccc(-c3cccc(N(c4ccc5c(c4)C(C)(C)c4ccccc4-5)c4ccc5c(c4)C4(c6ccccc6-c6ccccc64)c4ccccc4-5)c3)cc21]

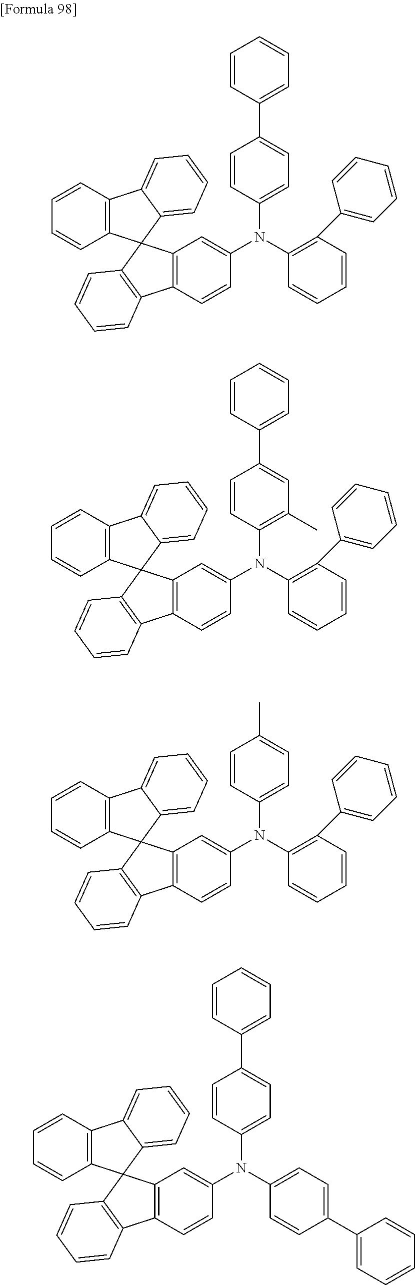 [SMILES: c1ccc(-c2ccc(N(c3ccc4c(c3)C3(c5ccccc5-c5ccccc53)c3ccccc3-4)c3ccccc3-c3ccccc3)cc2)cc1 ; Cc1cc(-c2ccccc2)ccc1N(c1ccc2c(c1)C1(c3ccccc3-c3ccccc31)c1ccccc1-2)c1ccccc1-c1ccccc1 ; Cc1ccc(N(c2ccc3c(c2)C2(c4ccccc4-c4ccccc42)c2ccccc2-3)c2ccccc2-c2ccccc2)cc1 ; c1ccc(-c2ccc(N(c3ccc(-c4ccccc4)cc3)c3ccc4c(c3)C3(c5ccccc5-c5ccccc53)c3ccccc3-4)cc2)cc1]

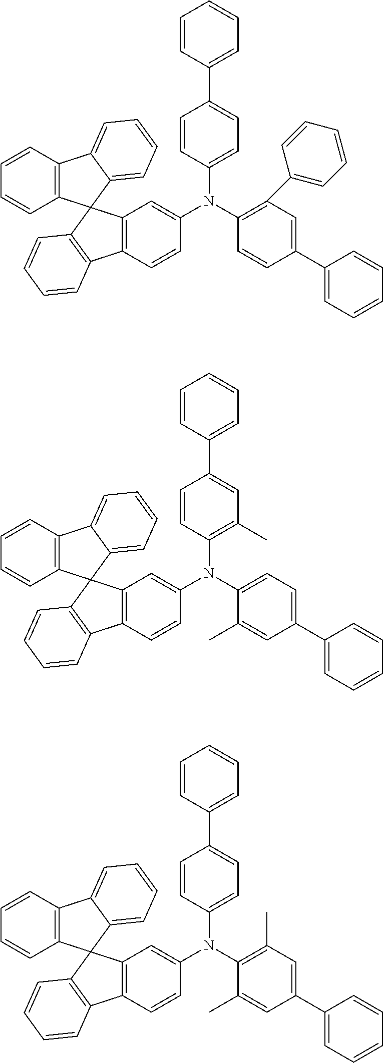 [SMILES: c1ccc(-c2ccc(N(c3ccc4c(c3)C3(c5ccccc5-c5ccccc53)c3ccccc3-4)c3ccc(-c4ccccc4)cc3-c3ccccc3)cc2)cc1 ; Cc1cc(-c2ccccc2)ccc1N(c1ccc2c(c1)C1(c3ccccc3-c3ccccc31)c1ccccc1-2)c1ccc(-c2ccccc2)cc1C ; Cc1cc(-c2ccccc2)cc(C)c1N(c1ccc(-c2ccccc2)cc1)c1ccc2c(c1)C1(c3ccccc3-c3ccccc31)c1ccccc1-2]

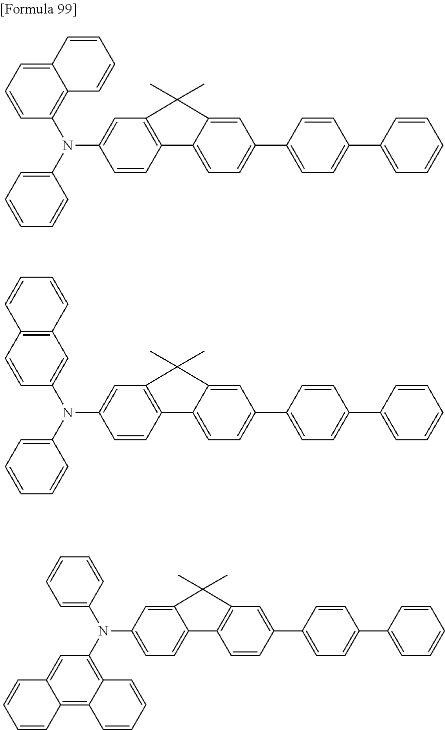 [SMILES: CC1(C)c2cc(-c3ccc(-c4ccccc4)cc3)ccc2-c2ccc(N(c3ccccc3)c3cccc4ccccc34)cc21 ; CC1(C)c2cc(-c3ccc(-c4ccccc4)cc3)ccc2-c2ccc(N(c3ccccc3)c3ccc4ccccc4c3)cc21 ; CC1(C)c2cc(-c3ccc(-c4ccccc4)cc3)ccc2-c2ccc(N(c3ccccc3)c3cc4ccccc4c4ccccc34)cc21]

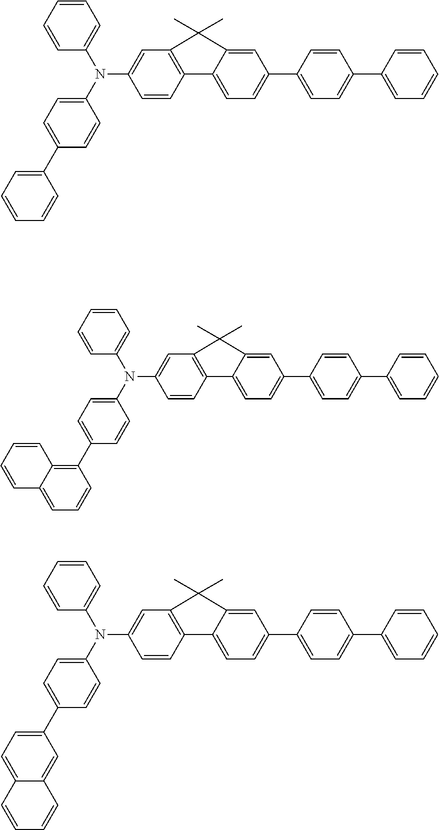 [SMILES: CC1(C)c2cc(-c3ccc(-c4ccccc4)cc3)ccc2-c2ccc(N(c3ccccc3)c3ccc(-c4ccccc4)cc3)cc21 ; CC1(C)c2cc(-c3ccc(-c4ccccc4)cc3)ccc2-c2ccc(N(c3ccccc3)c3ccc(-c4cccc5ccccc45)cc3)cc21 ; CC1(C)c2cc(-c3ccc(-c4ccccc4)cc3)ccc2-c2ccc(N(c3ccccc3)c3ccc(-c4ccc5ccccc5c4)cc3)cc21]

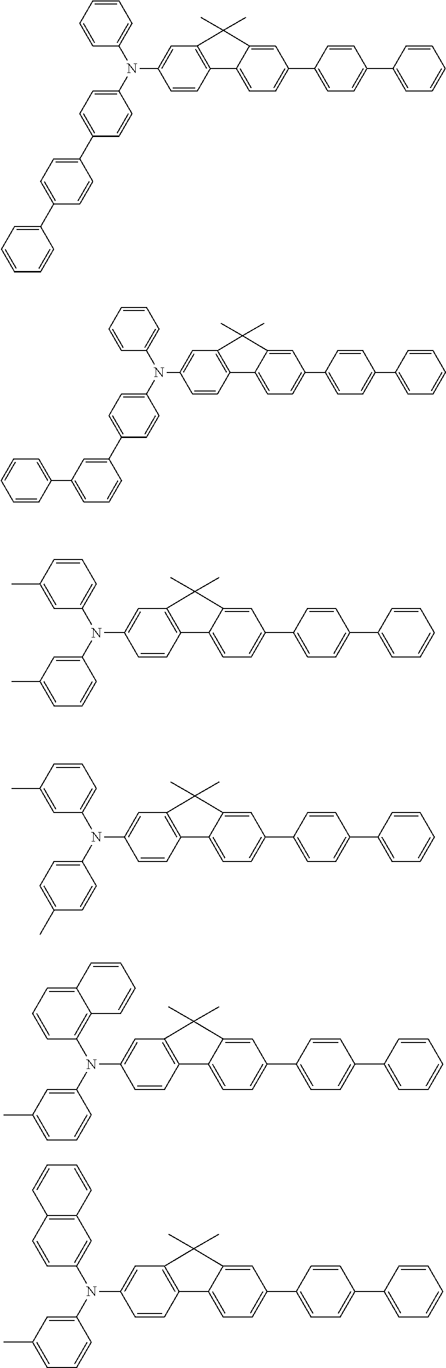

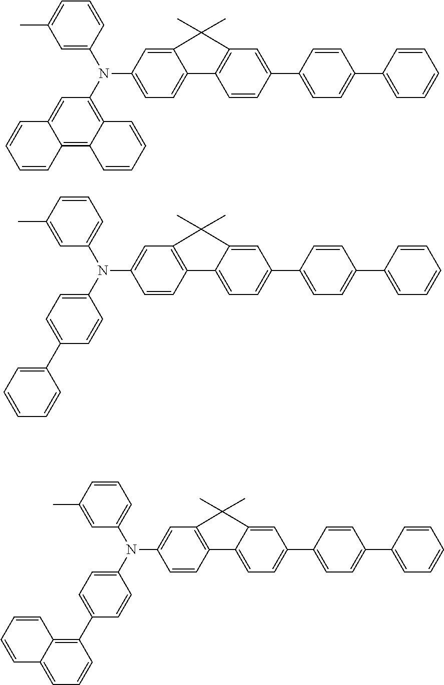

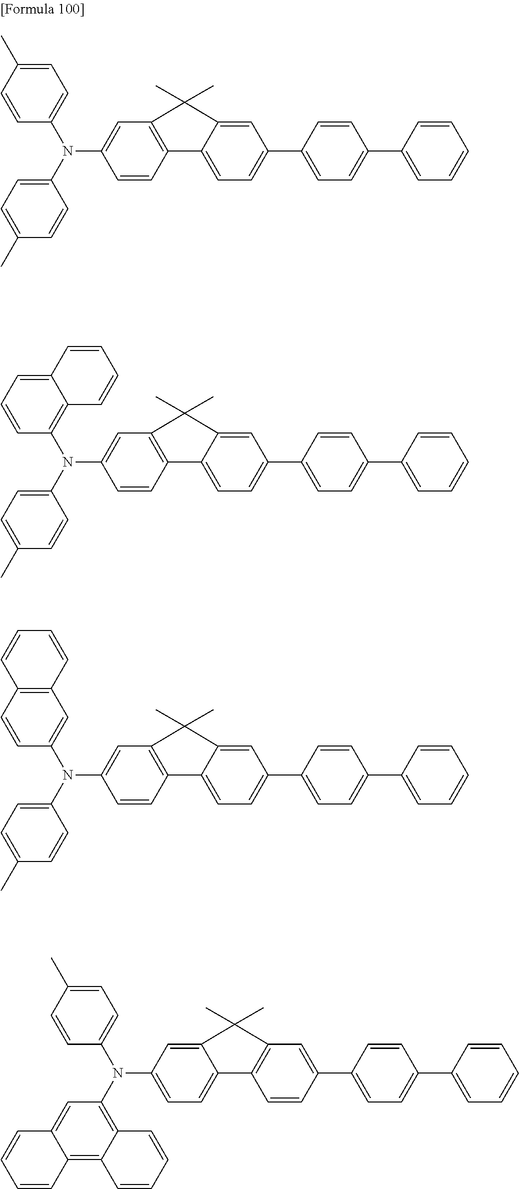 [SMILES: Cc1ccc(N(c2ccc(C)cc2)c2ccc3c(c2)C(C)(C)c2cc(-c4ccc(-c5ccccc5)cc4)ccc2-3)cc1 ; Cc1ccc(N(c2ccc3c(c2)C(C)(C)c2cc(-c4ccc(-c5ccccc5)cc4)ccc2-3)c2cccc3ccccc23)cc1 ; Cc1ccc(N(c2ccc3c(c2)C(C)(C)c2cc(-c4ccc(-c5ccccc5)cc4)ccc2-3)c2ccc3ccccc3c2)cc1 ; Cc1ccc(N(c2ccc3c(c2)C(C)(C)c2cc(-c4ccc(-c5ccccc5)cc4)ccc2-3)c2cc3ccccc3c3ccccc23)cc1]

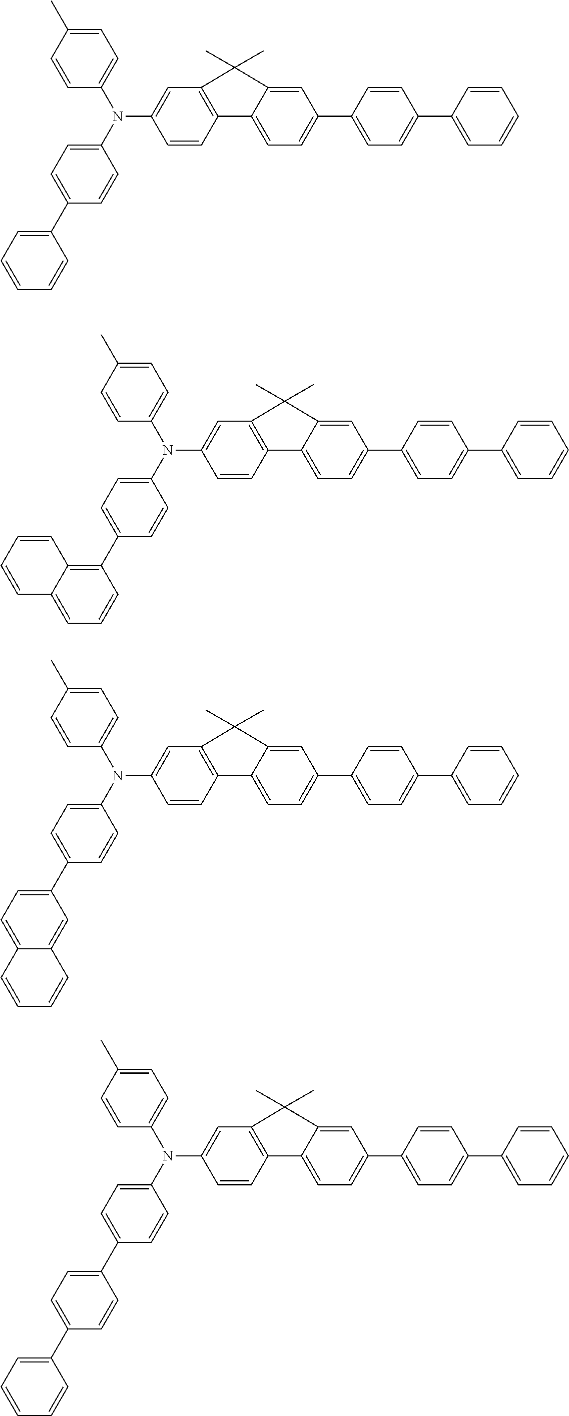

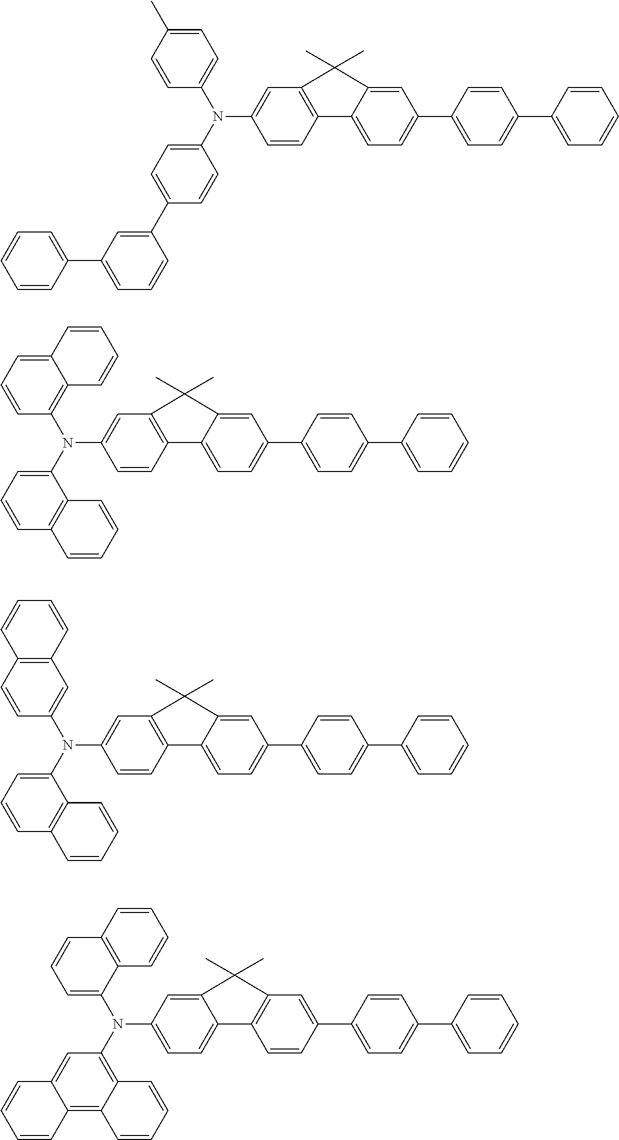

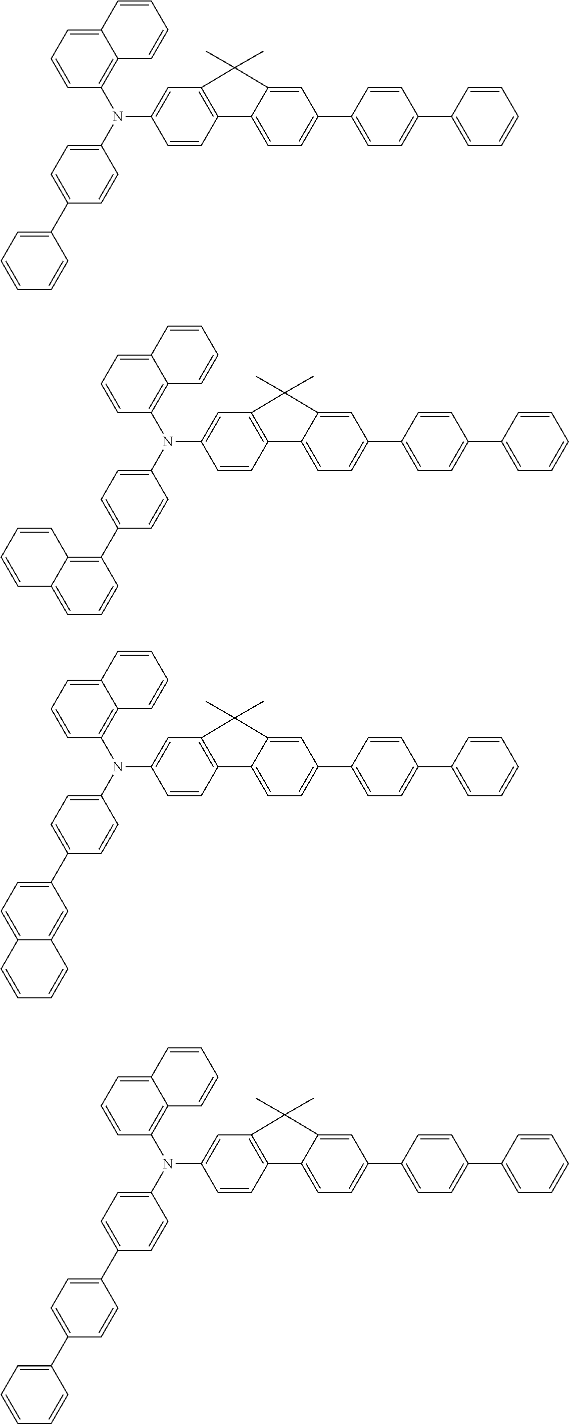

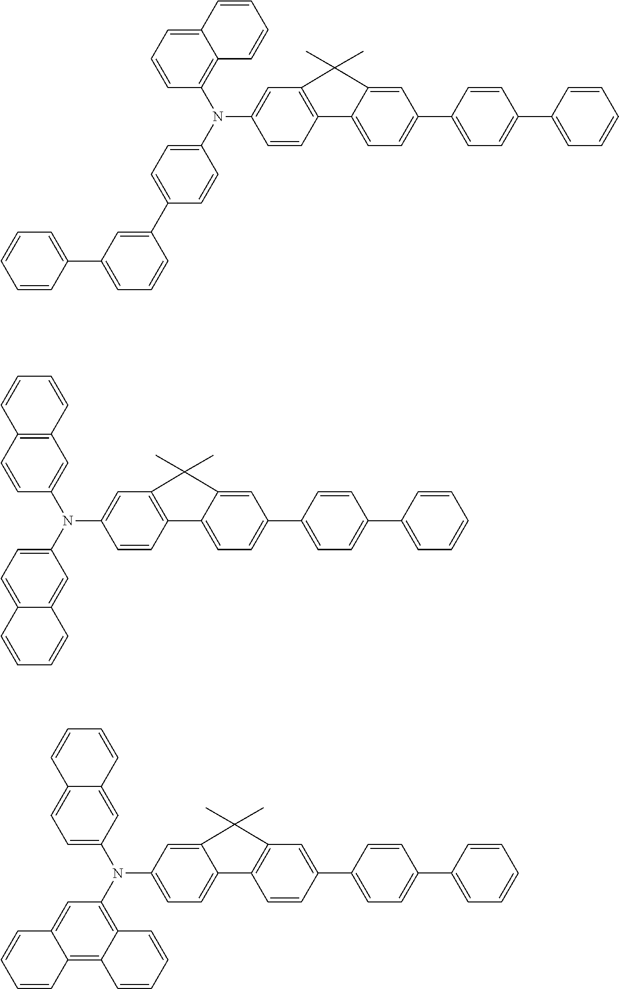 [SMILES: CC1(C)c2cc(-c3ccc(-c4ccccc4)cc3)ccc2-c2ccc(N(c3ccc(-c4cccc(-c5ccccc5)c4)cc3)c3cccc4ccccc34)cc21 ; CC1(C)c2cc(-c3ccc(-c4ccccc4)cc3)ccc2-c2ccc(N(c3ccc4ccccc4c3)c3ccc4ccccc4c3)cc21 ; CC1(C)c2cc(-c3ccc(-c4ccccc4)cc3)ccc2-c2ccc(N(c3ccc4ccccc4c3)c3cc4ccccc4c4ccccc34)cc21]

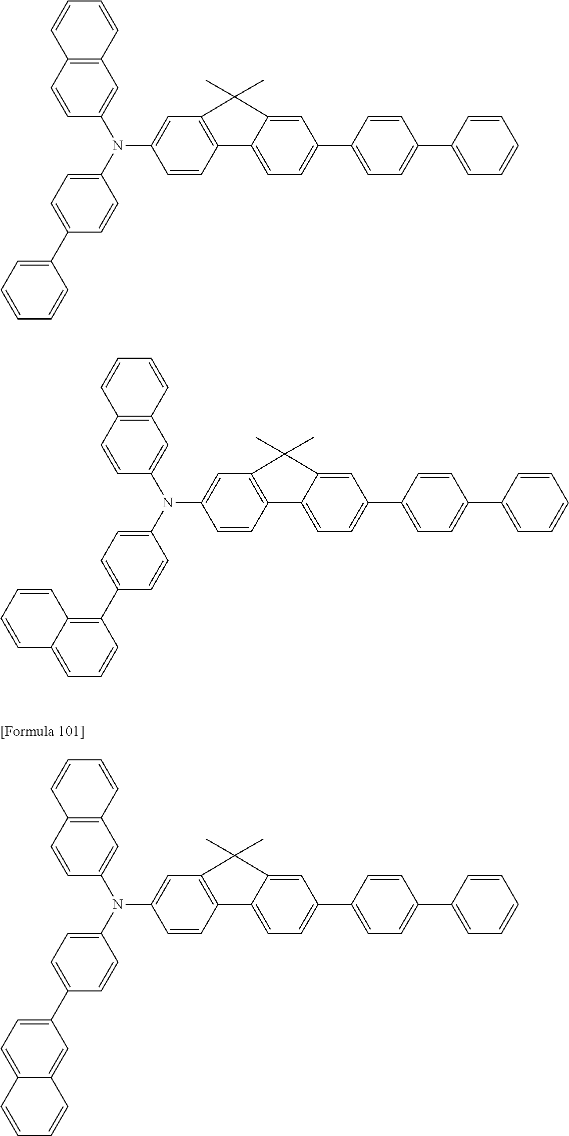 [SMILES: CC1(C)c2cc(-c3ccc(-c4ccccc4)cc3)ccc2-c2ccc(N(c3ccc(-c4ccccc4)cc3)c3ccc4ccccc4c3)cc21 ; CC1(C)c2cc(-c3ccc(-c4ccccc4)cc3)ccc2-c2ccc(N(c3ccc(-c4cccc5ccccc45)cc3)c3ccc4ccccc4c3)cc21 ; CC1(C)c2cc(-c3ccc(-c4ccccc4)cc3)ccc2-c2ccc(N(c3ccc(-c4ccc5ccccc5c4)cc3)c3ccc4ccccc4c3)cc21]

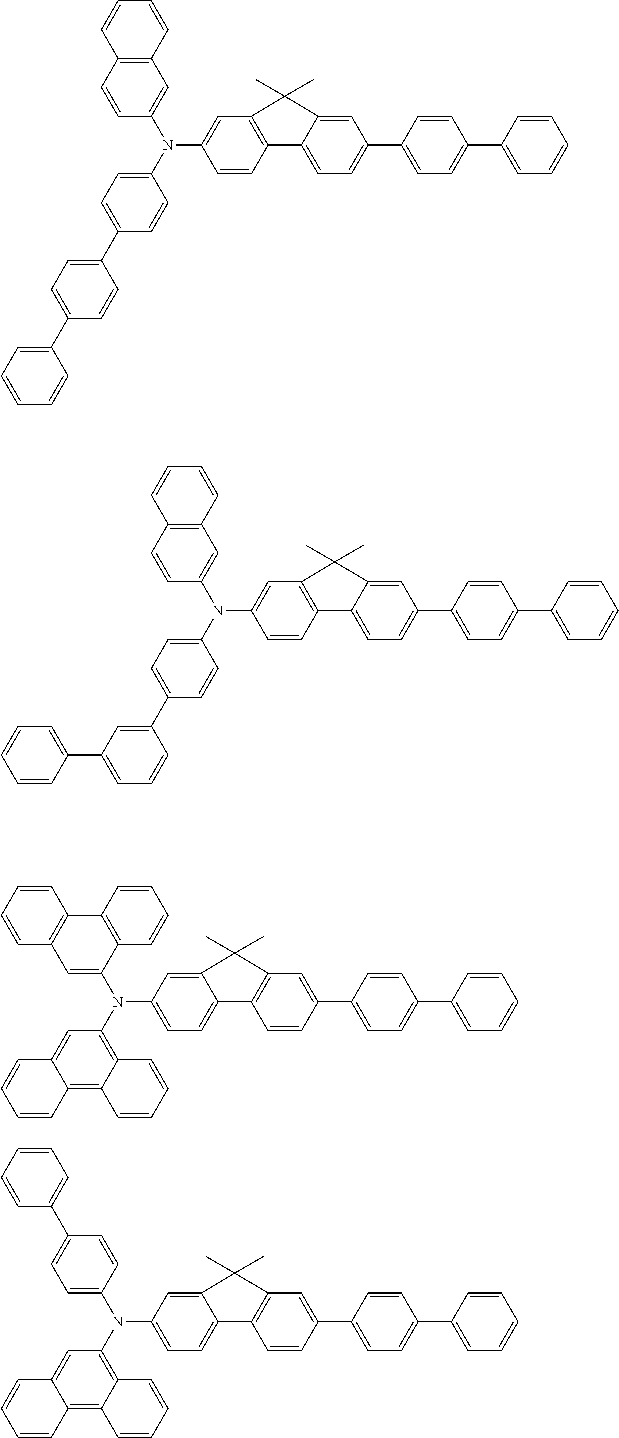

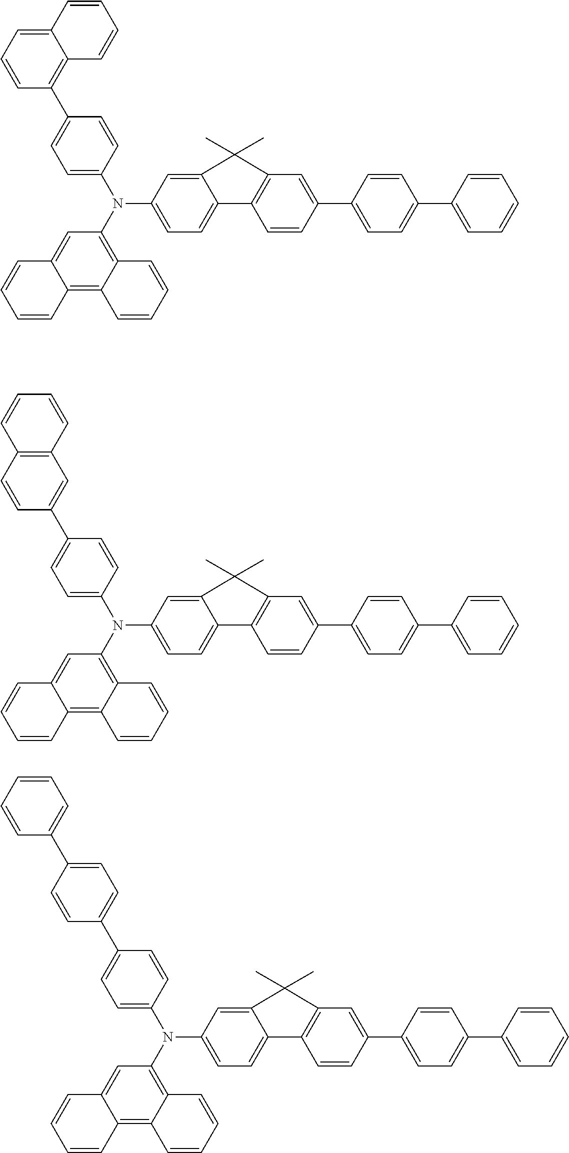

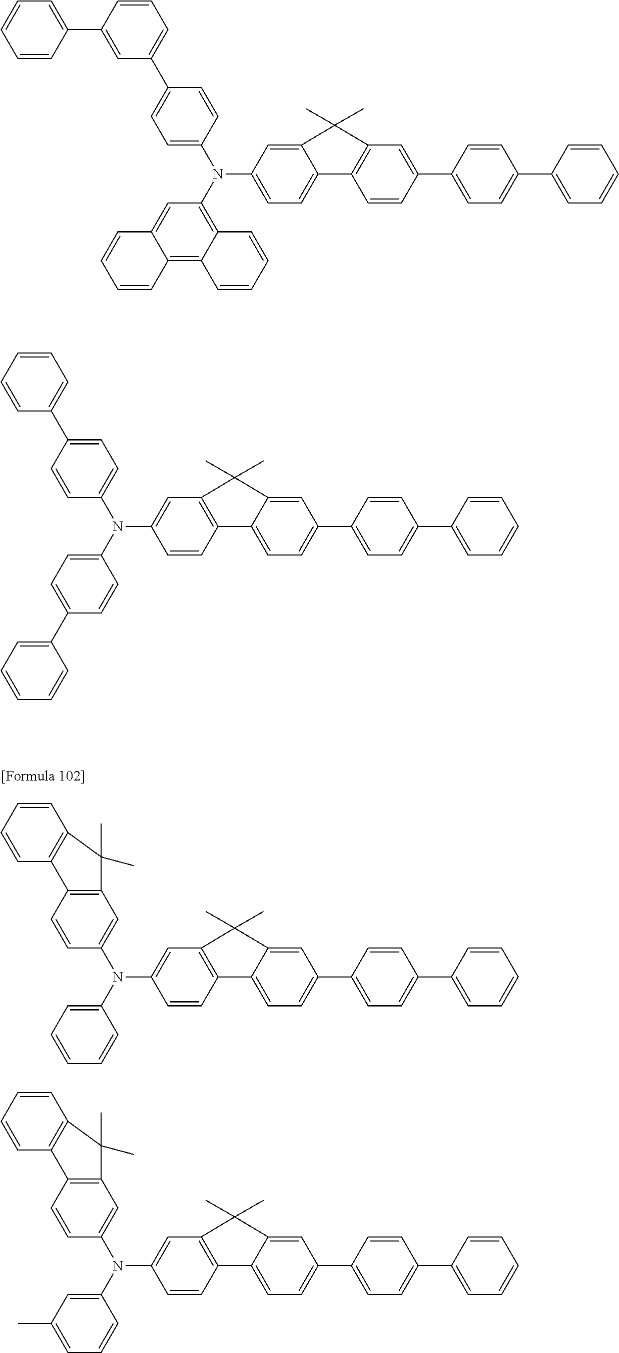

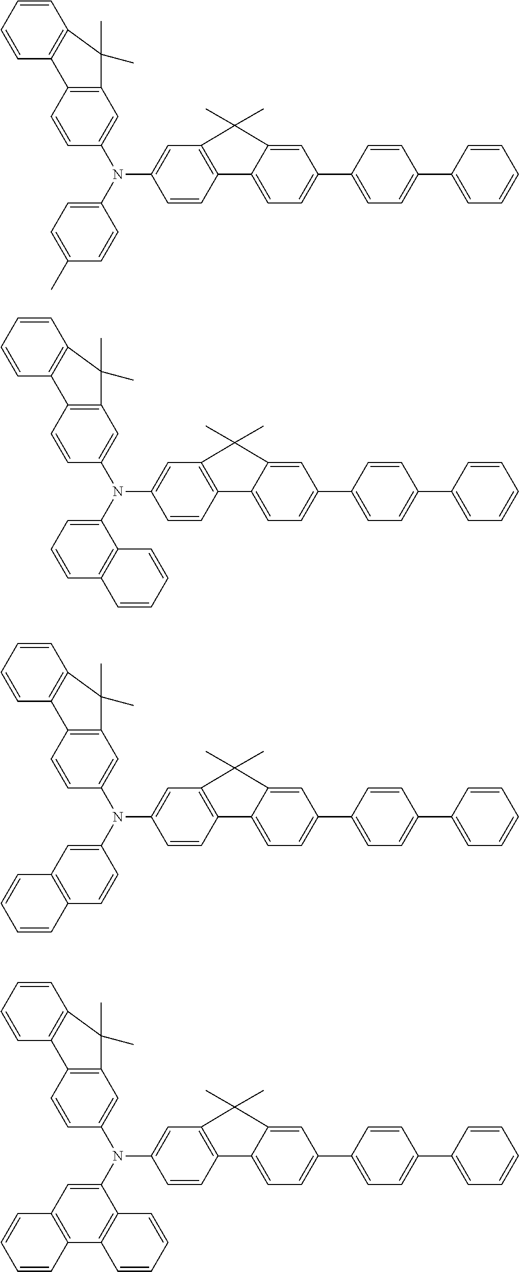

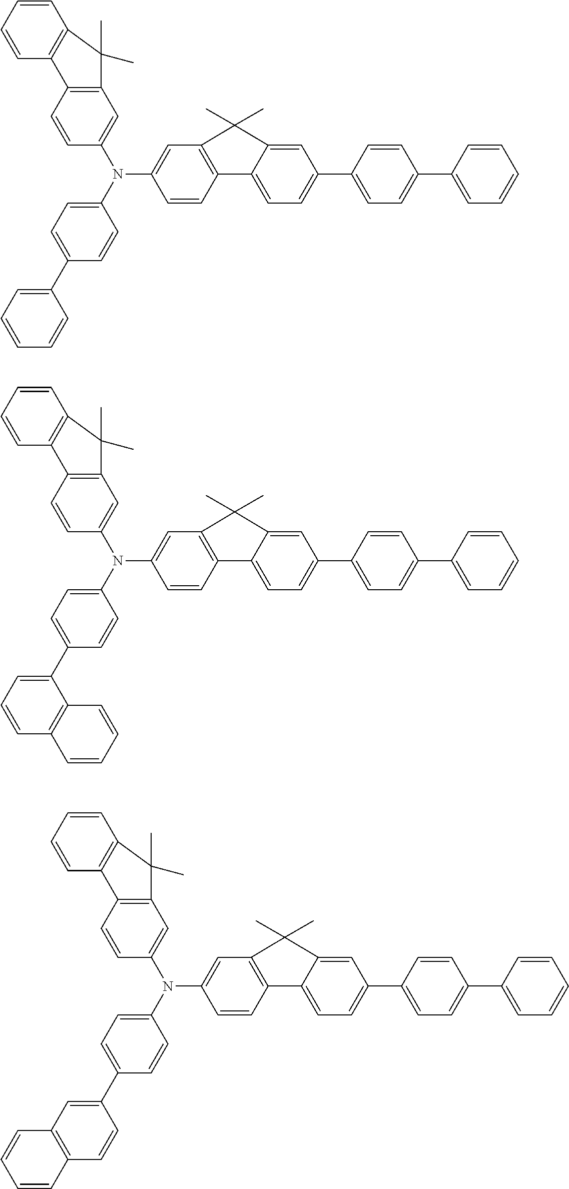

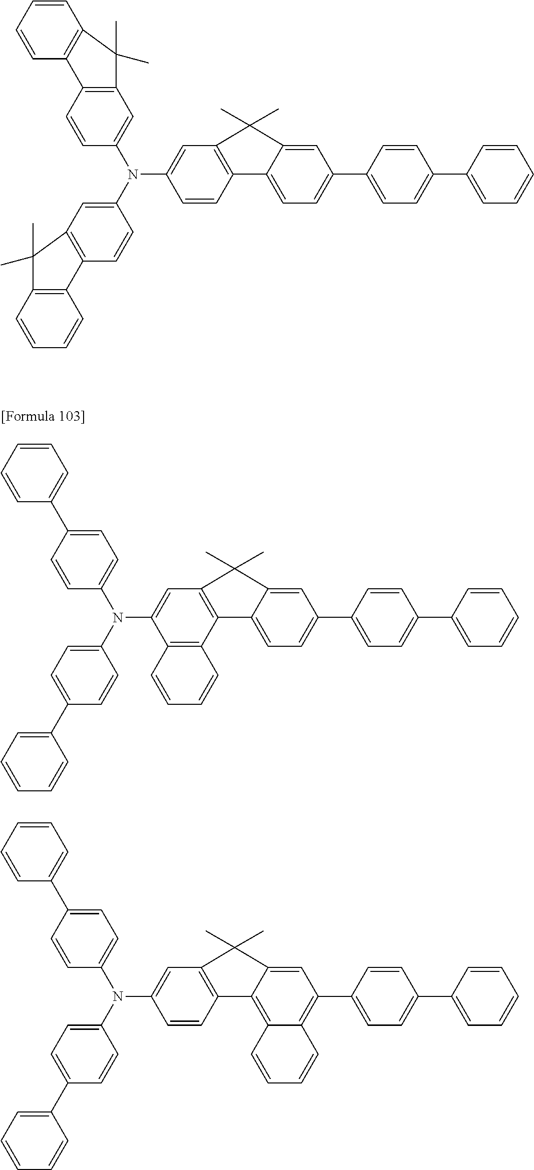

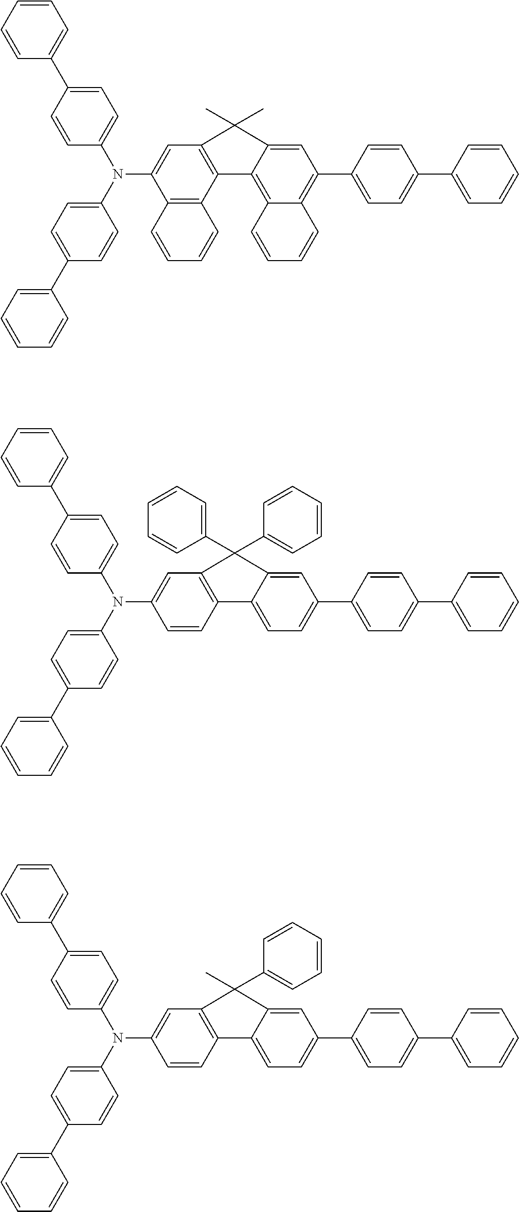 [SMILES: CC1(C)c2cc(-c3ccc(-c4ccccc4)cc3)c3ccccc3c2-c2c1cc(N(c1ccc(-c3ccccc3)cc1)c1ccc(-c3ccccc3)cc1)c1ccccc21 ; c1ccc(-c2ccc(-c3ccc4c(c3)C(c3ccccc3)(c3ccccc3)c3cc(N(c5ccc(-c6ccccc6)cc5)c5ccc(-c6ccccc6)cc5)ccc3-4)cc2)cc1 ; CC1(c2ccccc2)c2cc(-c3ccc(-c4ccccc4)cc3)ccc2-c2ccc(N(c3ccc(-c4ccccc4)cc3)c3ccc(-c4ccccc4)cc3)cc21]

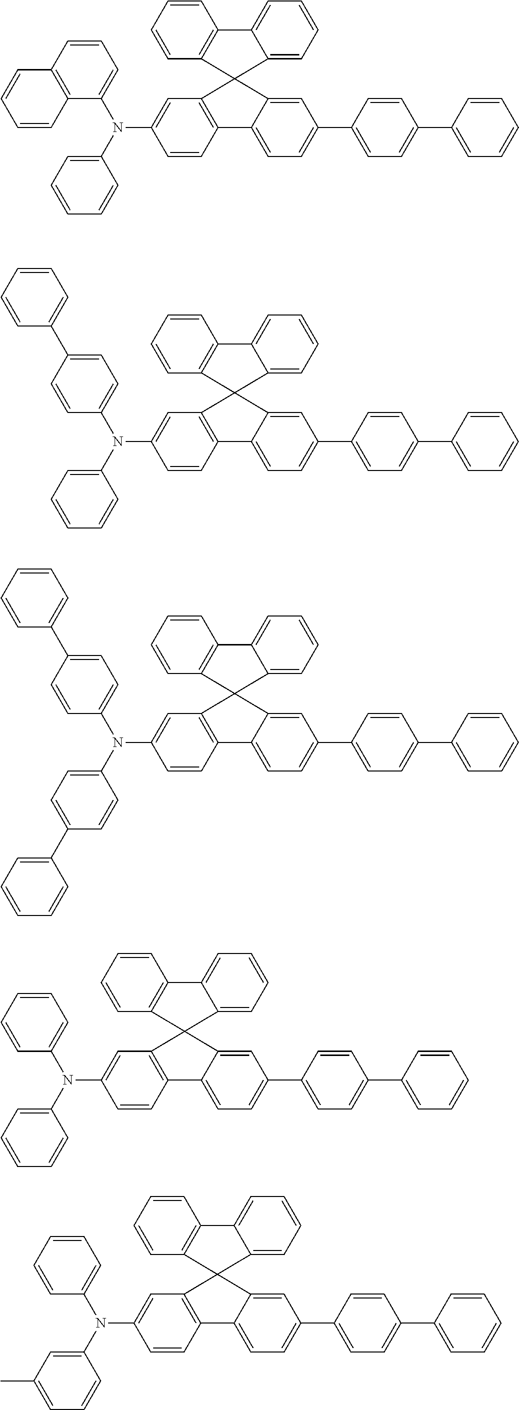

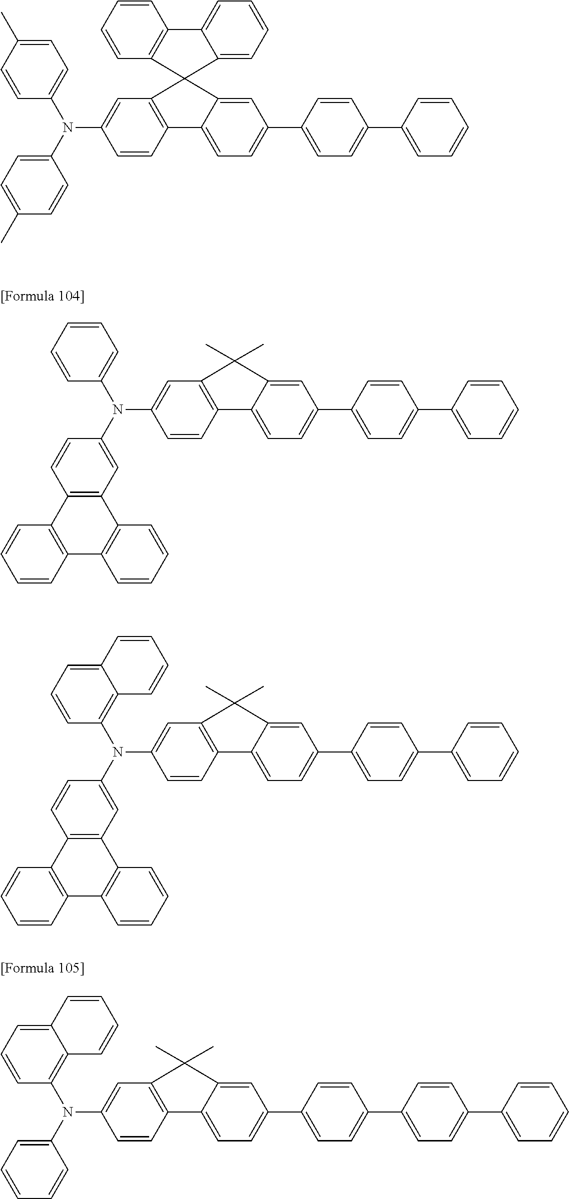 [SMILES: Cc1ccc(N(c2ccc(C)cc2)c2ccc3c(c2)C2(c4ccccc4-c4ccccc42)c2cc(-c4ccc(-c5ccccc5)cc4)ccc2-3)cc1 ; CC1(C)c2cc(-c3ccc(-c4ccccc4)cc3)ccc2-c2ccc(N(c3ccccc3)c3ccc4c5ccccc5c5ccccc5c4c3)cc21 ; CC1(C)c2cc(-c3ccc(-c4ccccc4)cc3)ccc2-c2ccc(N(c3ccc4c5ccccc5c5ccccc5c4c3)c3cccc4ccccc34)cc21 ; CC1(C)c2cc(-c3ccc(-c4ccc(-c5ccccc5)cc4)cc3)ccc2-c2ccc(N(c3ccccc3)c3cccc4ccccc34)cc21]

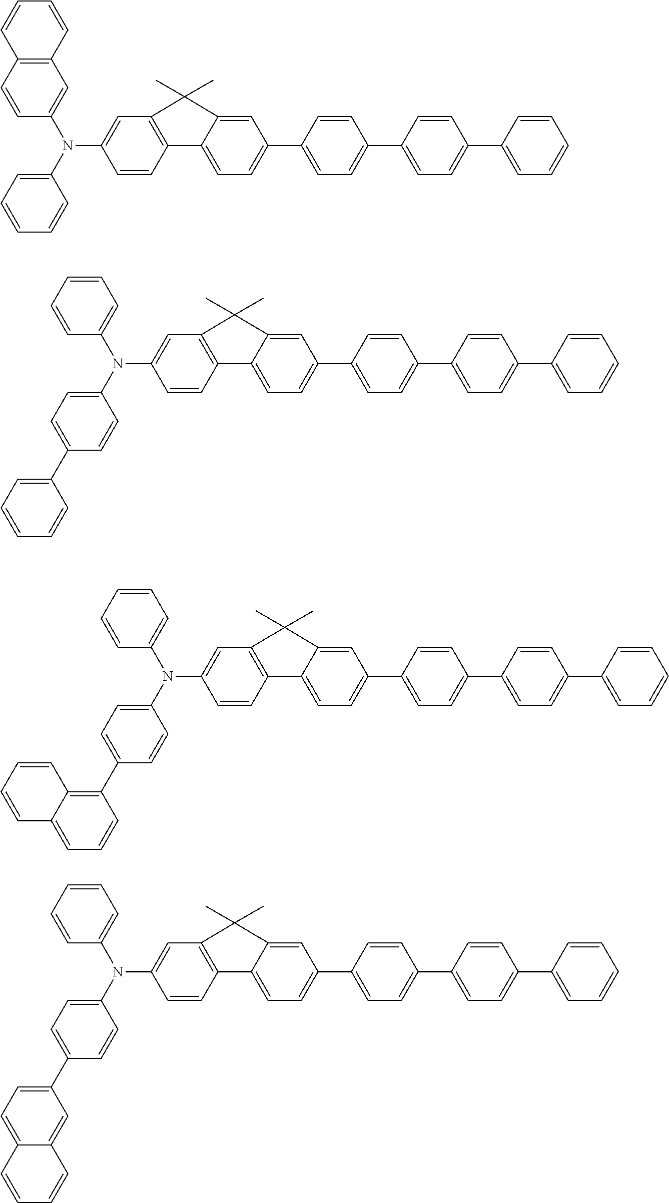

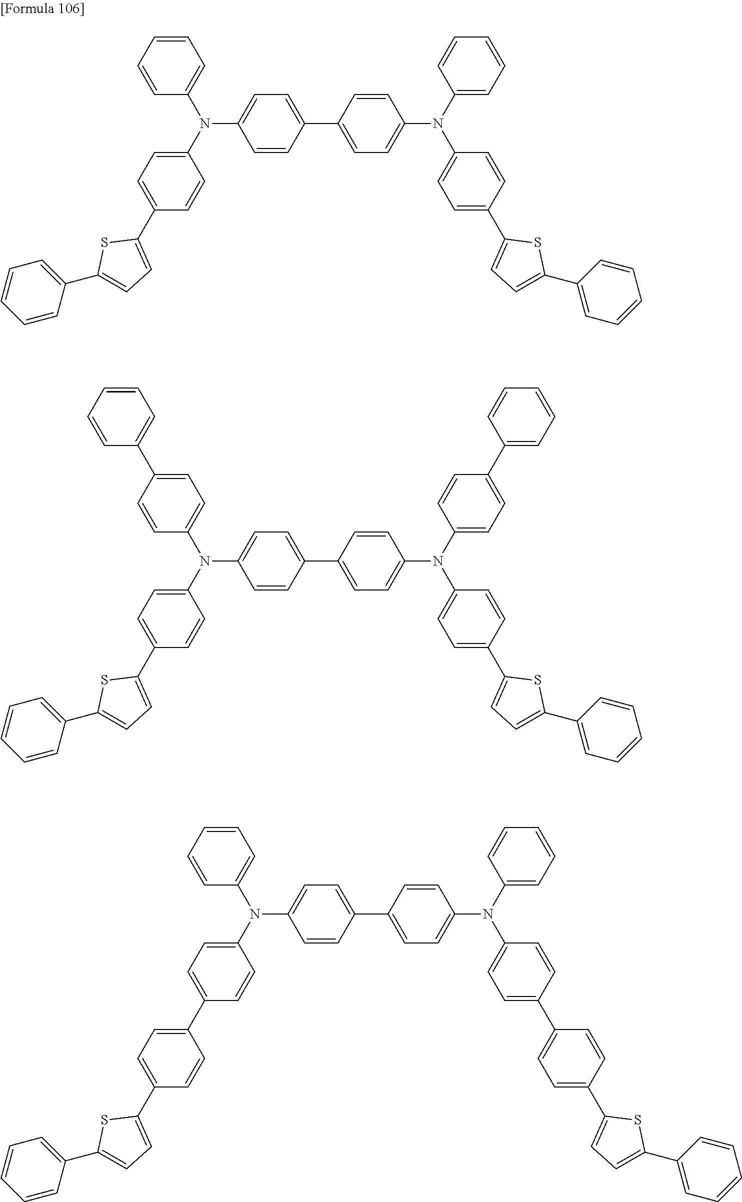 [SMILES: c1ccc(-c2ccc(-c3ccc(N(c4ccccc4)c4ccc(-c5ccc(N(c6ccccc6)c6ccc(-c7ccc(-c8ccccc8)s7)cc6)cc5)cc4)cc3)s2)cc1 ; c1ccc(-c2ccc(N(c3ccc(-c4ccc(N(c5ccc(-c6ccccc6)cc5)c5ccc(-c6ccc(-c7ccccc7)s6)cc5)cc4)cc3)c3ccc(-c4ccc(-c5ccccc5)s4)cc3)cc2)cc1 ; c1ccc(-c2ccc(-c3ccc(-c4ccc(N(c5ccccc5)c5ccc(-c6ccc(N(c7ccccc7)c7ccc(-c8ccc(-c9ccc(-c%10ccccc%10)s9)cc8)cc7)cc6)cc5)cc4)cc3)s2)cc1]

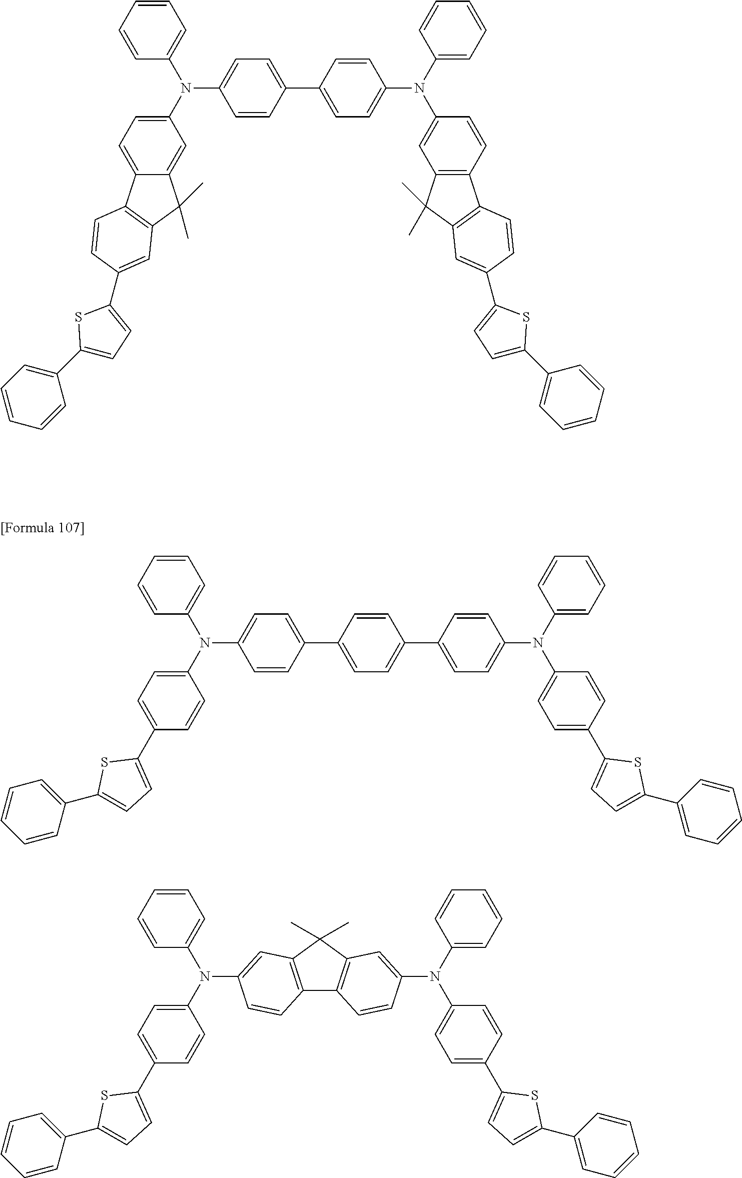 [SMILES: CC1(C)c2cc(-c3ccc(-c4ccccc4)s3)ccc2-c2ccc(N(c3ccccc3)c3ccc(-c4ccc(N(c5ccccc5)c5ccc6c(c5)C(C)(C)c5cc(-c7ccc(-c8ccccc8)s7)ccc5-6)cc4)cc3)cc21 ; c1ccc(-c2ccc(-c3ccc(N(c4ccccc4)c4ccc(-c5ccc(-c6ccc(N(c7ccccc7)c7ccc(-c8ccc(-c9ccccc9)s8)cc7)cc6)cc5)cc4)cc3)s2)cc1 ; CC1(C)c2cc(N(c3ccccc3)c3ccc(-c4ccc(-c5ccccc5)s4)cc3)ccc2-c2ccc(N(c3ccccc3)c3ccc(-c4ccc(-c5ccccc5)s4)cc3)cc21]

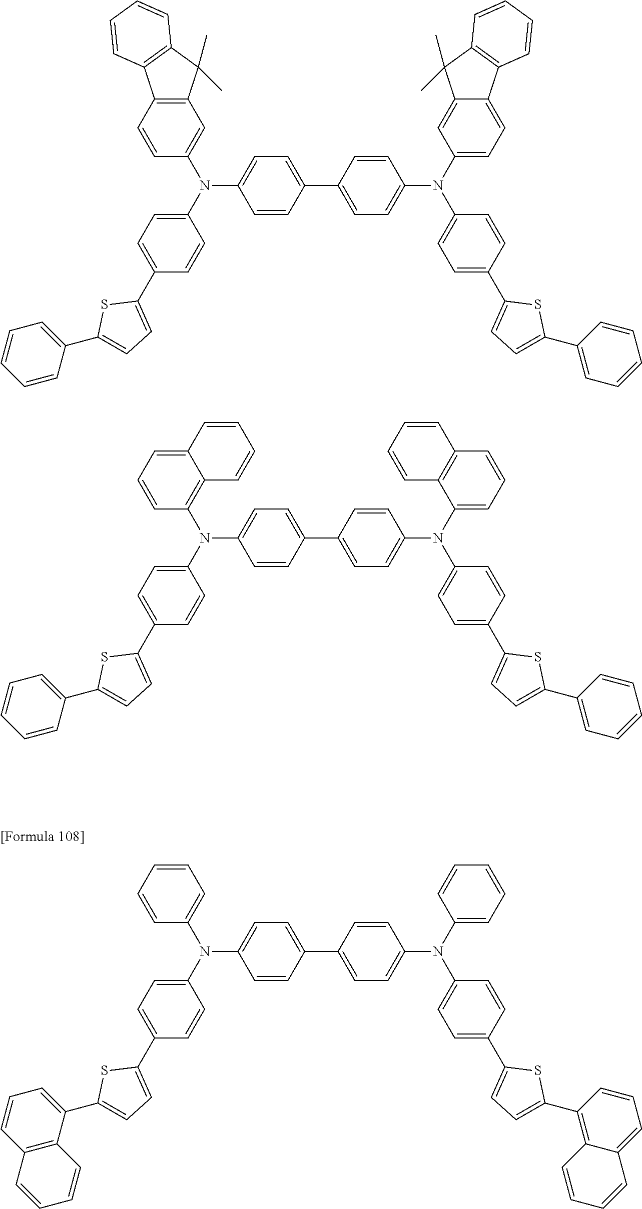 [SMILES: CC1(C)c2ccccc2-c2ccc(N(c3ccc(-c4ccc(N(c5ccc(-c6ccc(-c7ccccc7)s6)cc5)c5ccc6c(c5)C(C)(C)c5ccccc5-6)cc4)cc3)c3ccc(-c4ccc(-c5ccccc5)s4)cc3)cc21 ; c1ccc(-c2ccc(-c3ccc(N(c4ccc(-c5ccc(N(c6ccc(-c7ccc(-c8ccccc8)s7)cc6)c6cccc7ccccc67)cc5)cc4)c4cccc5ccccc45)cc3)s2)cc1 ; c1ccc(N(c2ccc(-c3ccc(N(c4ccccc4)c4ccc(-c5ccc(-c6cccc7ccccc67)s5)cc4)cc3)cc2)c2ccc(-c3ccc(-c4cccc5ccccc45)s3)cc2)cc1]

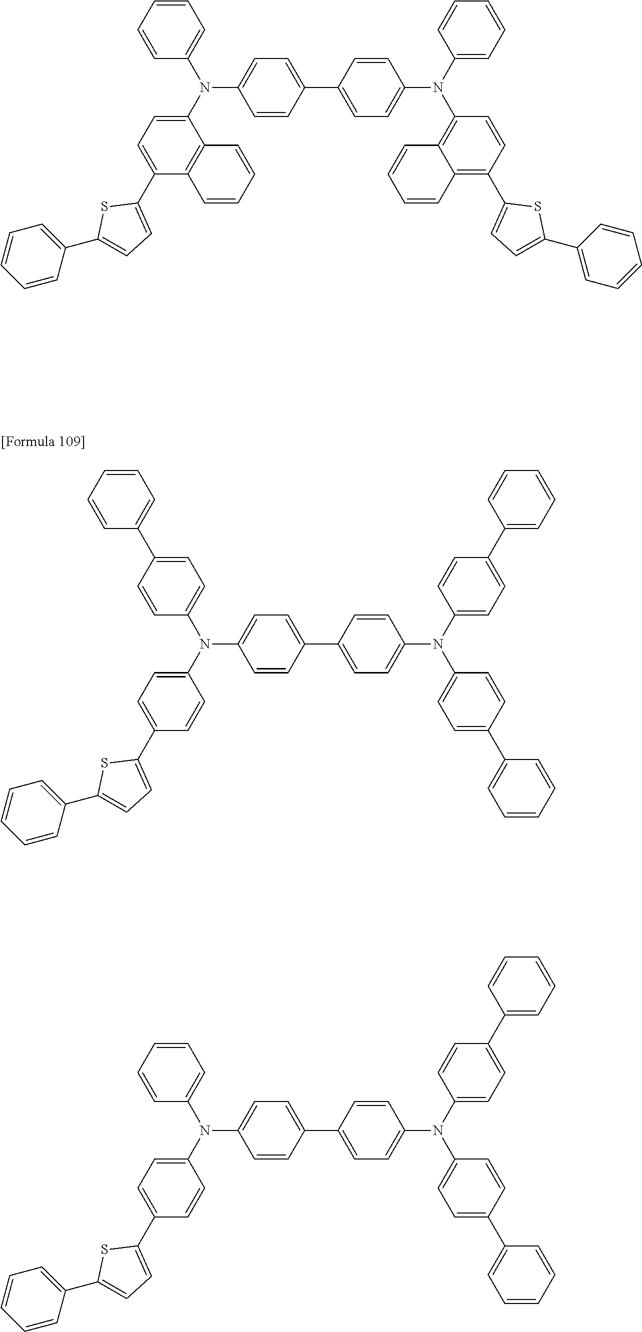 [SMILES: c1ccc(-c2ccc(-c3ccc(N(c4ccccc4)c4ccc(-c5ccc(N(c6ccccc6)c6ccc(-c7ccc(-c8ccccc8)s7)c7ccccc67)cc5)cc4)c4ccccc34)s2)cc1 ; c1ccc(-c2ccc(N(c3ccc(-c4ccccc4)cc3)c3ccc(-c4ccc(N(c5ccc(-c6ccccc6)cc5)c5ccc(-c6ccc(-c7ccccc7)s6)cc5)cc4)cc3)cc2)cc1 ; c1ccc(-c2ccc(N(c3ccc(-c4ccccc4)cc3)c3ccc(-c4ccc(N(c5ccccc5)c5ccc(-c6ccc(-c7ccccc7)s6)cc5)cc4)cc3)cc2)cc1]

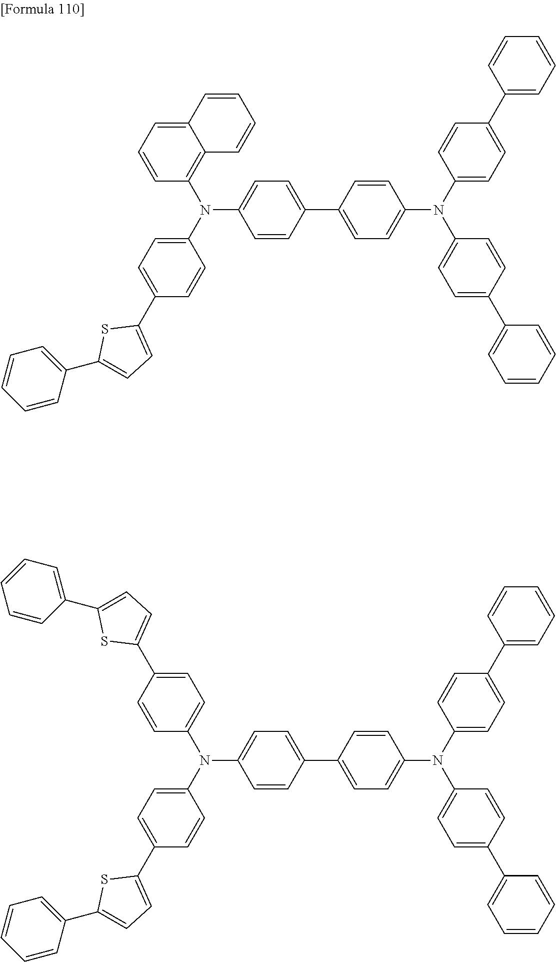 [SMILES: c1ccc(-c2ccc(N(c3ccc(-c4ccccc4)cc3)c3ccc(-c4ccc(N(c5ccc(-c6ccc(-c7ccccc7)s6)cc5)c5cccc6ccccc56)cc4)cc3)cc2)cc1 ; c1ccc(-c2ccc(N(c3ccc(-c4ccccc4)cc3)c3ccc(-c4ccc(N(c5ccc(-c6ccc(-c7ccccc7)s6)cc5)c5ccc(-c6ccc(-c7ccccc7)s6)cc5)cc4)cc3)cc2)cc1]

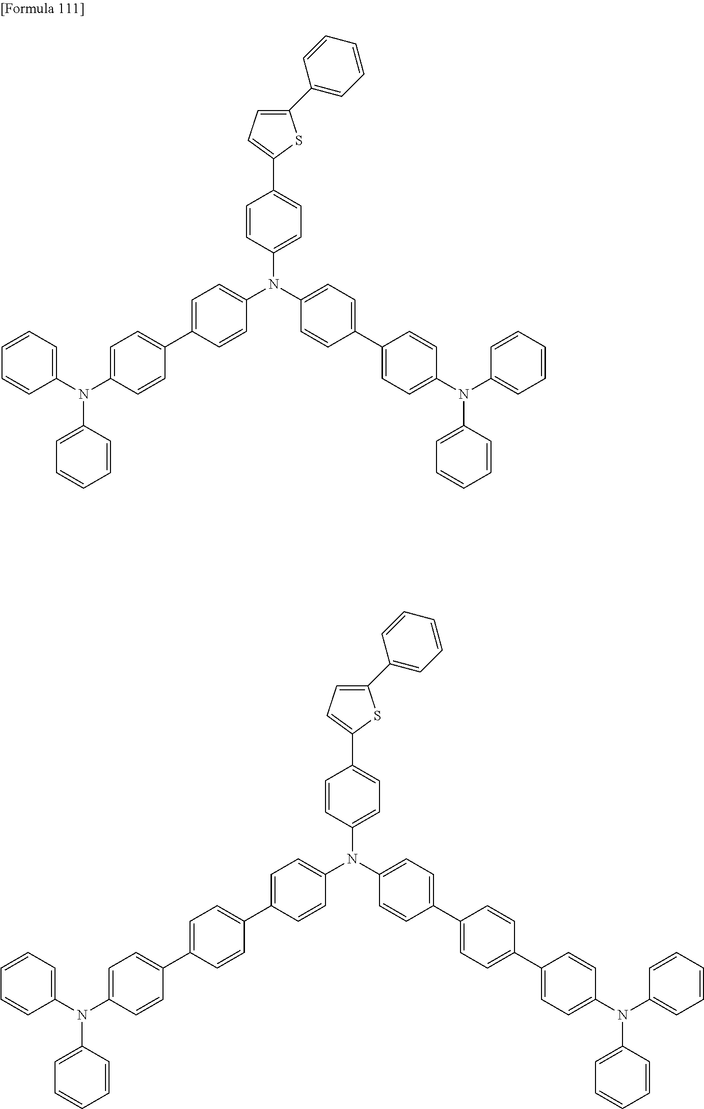 [SMILES: c1ccc(-c2ccc(-c3ccc(N(c4ccc(-c5ccc(N(c6ccccc6)c6ccccc6)cc5)cc4)c4ccc(-c5ccc(N(c6ccccc6)c6ccccc6)cc5)cc4)cc3)s2)cc1 ; c1ccc(-c2ccc(-c3ccc(N(c4ccc(-c5ccc(-c6ccc(N(c7ccccc7)c7ccccc7)cc6)cc5)cc4)c4ccc(-c5ccc(-c6ccc(N(c7ccccc7)c7ccccc7)cc6)cc5)cc4)cc3)s2)cc1]

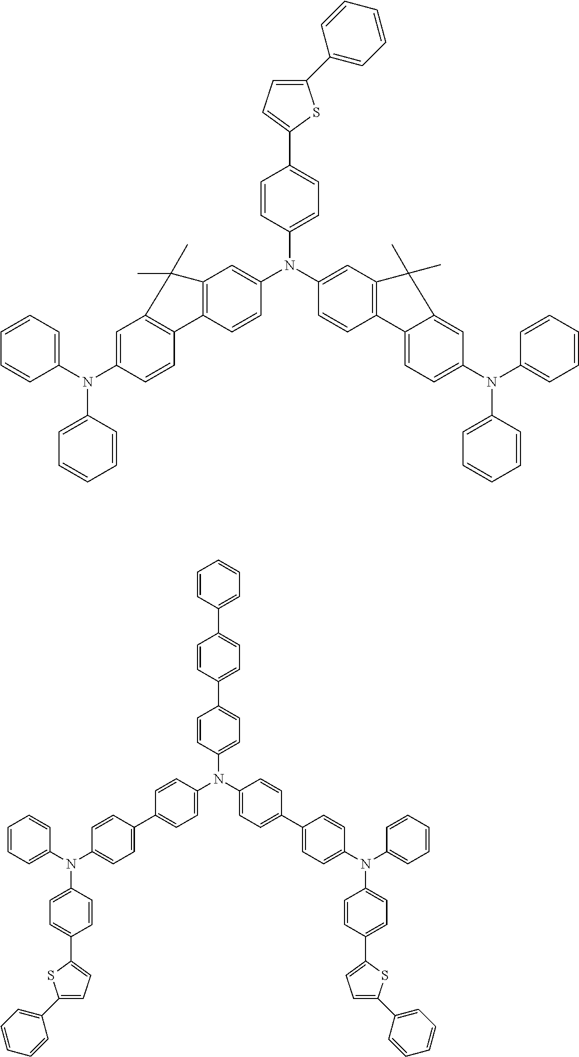 [SMILES: CC1(C)c2cc(N(c3ccccc3)c3ccccc3)ccc2-c2ccc(N(c3ccc(-c4ccc(-c5ccccc5)s4)cc3)c3ccc4c(c3)C(C)(C)c3cc(N(c5ccccc5)c5ccccc5)ccc3-4)cc21 ; c1ccc(-c2ccc(-c3ccc(N(c4ccc(-c5ccc(N(c6ccccc6)c6ccc(-c7ccc(-c8ccccc8)s7)cc6)cc5)cc4)c4ccc(-c5ccc(N(c6ccccc6)c6ccc(-c7ccc(-c8ccccc8)s7)cc6)cc5)cc4)cc3)cc2)cc1]

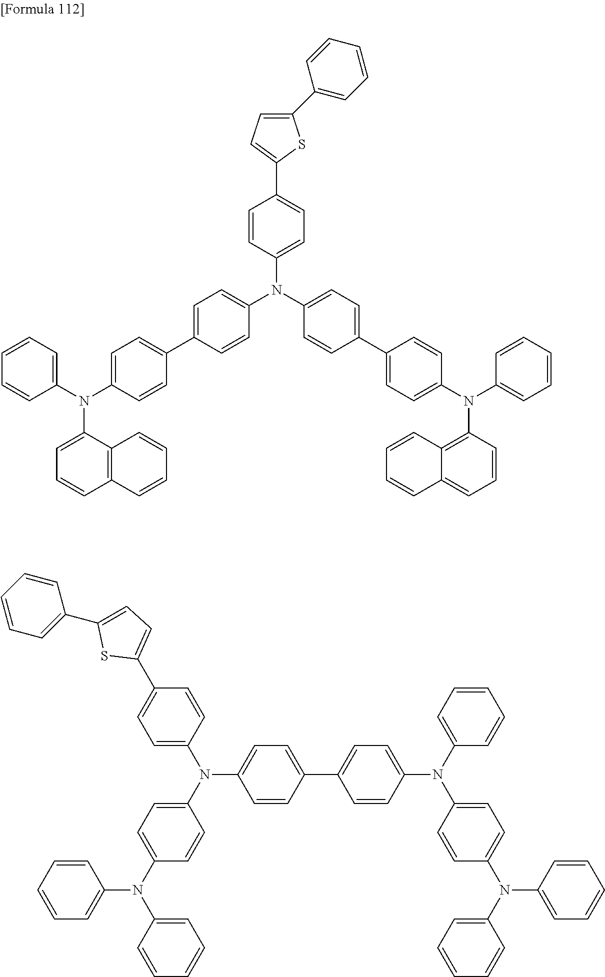 [SMILES: c1ccc(-c2ccc(-c3ccc(N(c4ccc(-c5ccc(N(c6ccccc6)c6cccc7ccccc67)cc5)cc4)c4ccc(-c5ccc(N(c6ccccc6)c6cccc7ccccc67)cc5)cc4)cc3)s2)cc1 ; c1ccc(-c2ccc(-c3ccc(N(c4ccc(-c5ccc(N(c6ccccc6)c6ccc(N(c7ccccc7)c7ccccc7)cc6)cc5)cc4)c4ccc(N(c5ccccc5)c5ccccc5)cc4)cc3)s2)cc1]

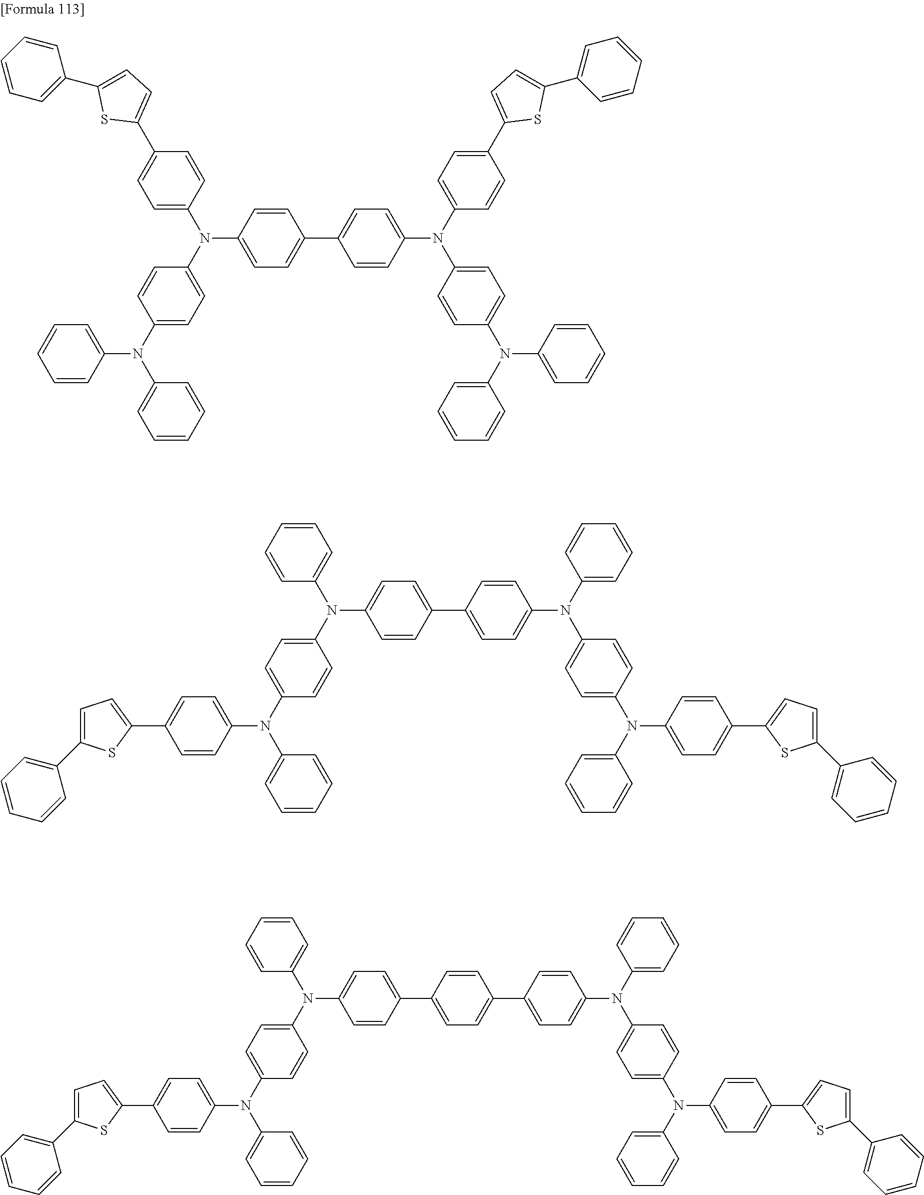 [SMILES: c1ccc(-c2ccc(-c3ccc(N(c4ccc(-c5ccc(N(c6ccc(-c7ccc(-c8ccccc8)s7)cc6)c6ccc(N(c7ccccc7)c7ccccc7)cc6)cc5)cc4)c4ccc(N(c5ccccc5)c5ccccc5)cc4)cc3)s2)cc1 ; c1ccc(-c2ccc(-c3ccc(N(c4ccccc4)c4ccc(N(c5ccccc5)c5ccc(-c6ccc(N(c7ccccc7)c7ccc(N(c8ccccc8)c8ccc(-c9ccc(-c%10ccccc%10)s9)cc8)cc7)cc6)cc5)cc4)cc3)s2)cc1 ; c1ccc(-c2ccc(-c3ccc(N(c4ccccc4)c4ccc(N(c5ccccc5)c5ccc(-c6ccc(-c7ccc(N(c8ccccc8)c8ccc(N(c9ccccc9)c9ccc(-c%10ccc(-c%11ccccc%11)s%10)cc9)cc8)cc7)cc6)cc5)cc4)cc3)s2)cc1]

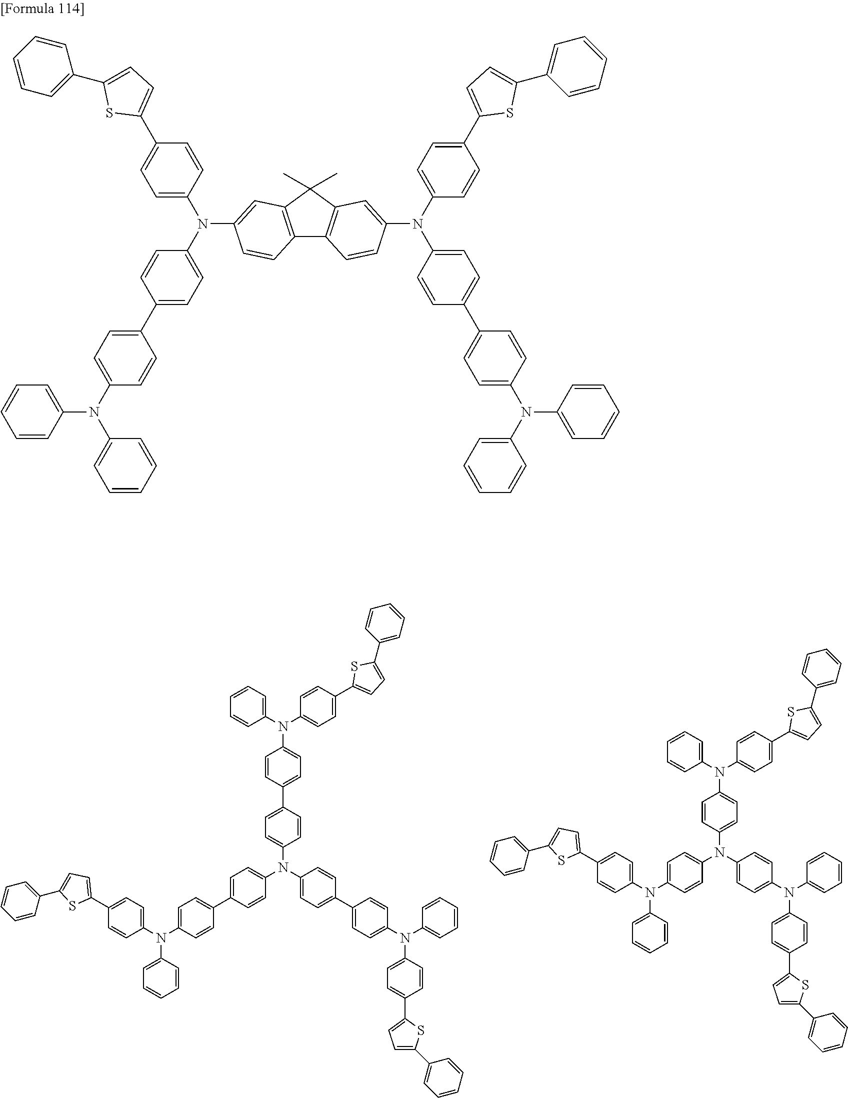

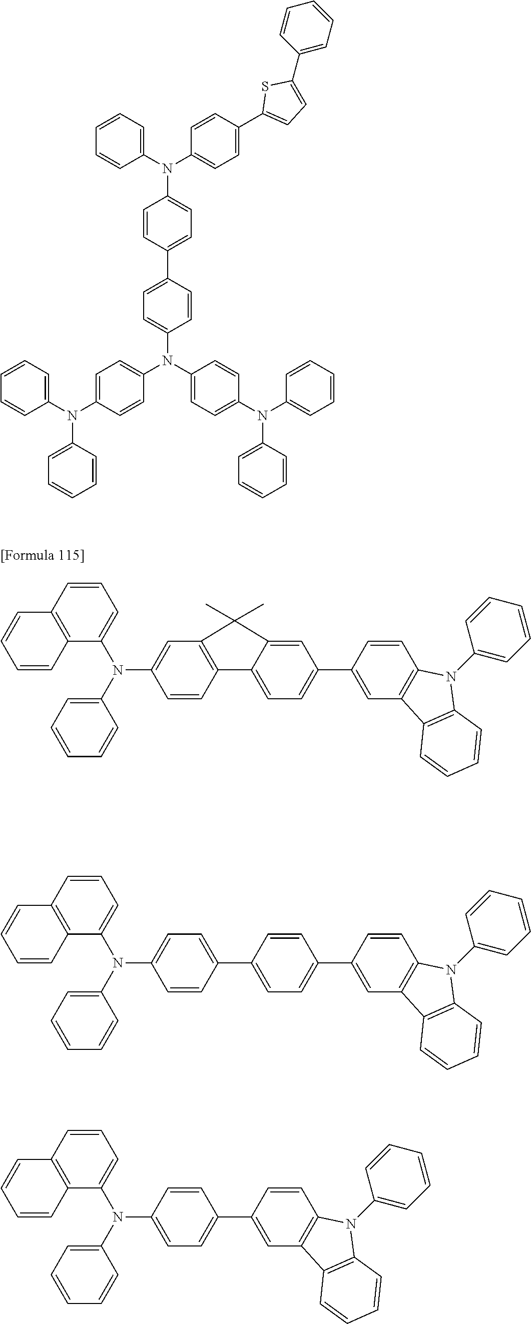 [SMILES: c1ccc(-c2ccc(-c3ccc(N(c4ccccc4)c4ccc(-c5ccc(N(c6ccc(N(c7ccccc7)c7ccccc7)cc6)c6ccc(N(c7ccccc7)c7ccccc7)cc6)cc5)cc4)cc3)s2)cc1 ; CC1(C)c2cc(-c3ccc4c(c3)c3ccccc3n4-c3ccccc3)ccc2-c2ccc(N(c3ccccc3)c3cccc4ccccc34)cc21 ; c1ccc(N(c2ccc(-c3ccc(-c4ccc5c(c4)c4ccccc4n5-c4ccccc4)cc3)cc2)c2cccc3ccccc23)cc1 ; c1ccc(N(c2ccc(-c3ccc4c(c3)c3ccccc3n4-c3ccccc3)cc2)c2cccc3ccccc23)cc1]

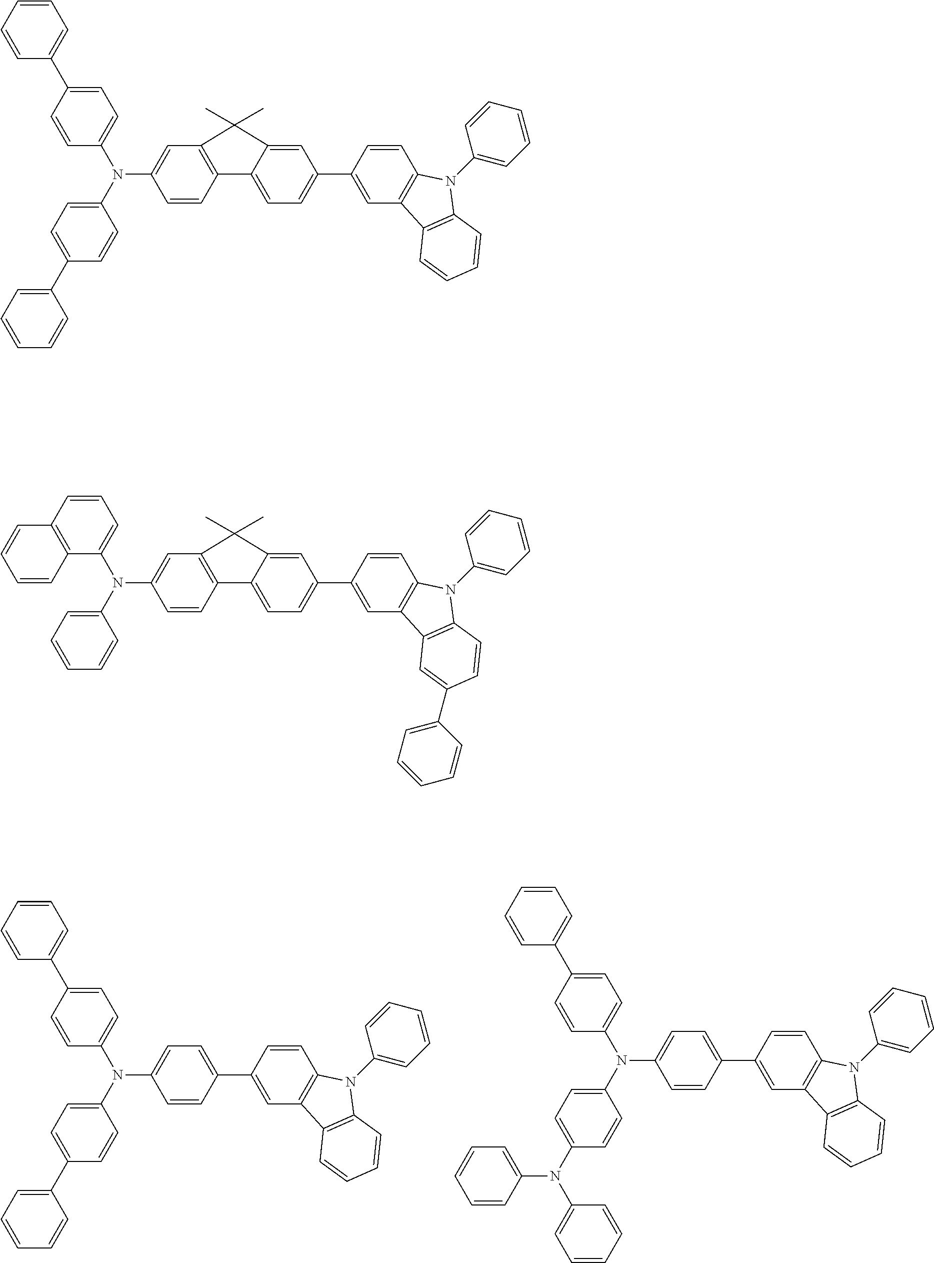 [SMILES: CC1(C)c2cc(-c3ccc4c(c3)c3ccccc3n4-c3ccccc3)ccc2-c2ccc(N(c3ccc(-c4ccccc4)cc3)c3ccc(-c4ccccc4)cc3)cc21 ; CC1(C)c2cc(-c3ccc4c(c3)c3cc(-c5ccccc5)ccc3n4-c3ccccc3)ccc2-c2ccc(N(c3ccccc3)c3cccc4ccccc34)cc21 ; c1ccc(-c2ccc(N(c3ccc(-c4ccccc4)cc3)c3ccc(-c4ccc5c(c4)c4ccccc4n5-c4ccccc4)cc3)cc2)cc1 ; c1ccc(-c2ccc(N(c3ccc(-c4ccc5c(c4)c4ccccc4n5-c4ccccc4)cc3)c3ccc(N(c4ccccc4)c4ccccc4)cc3)cc2)cc1]

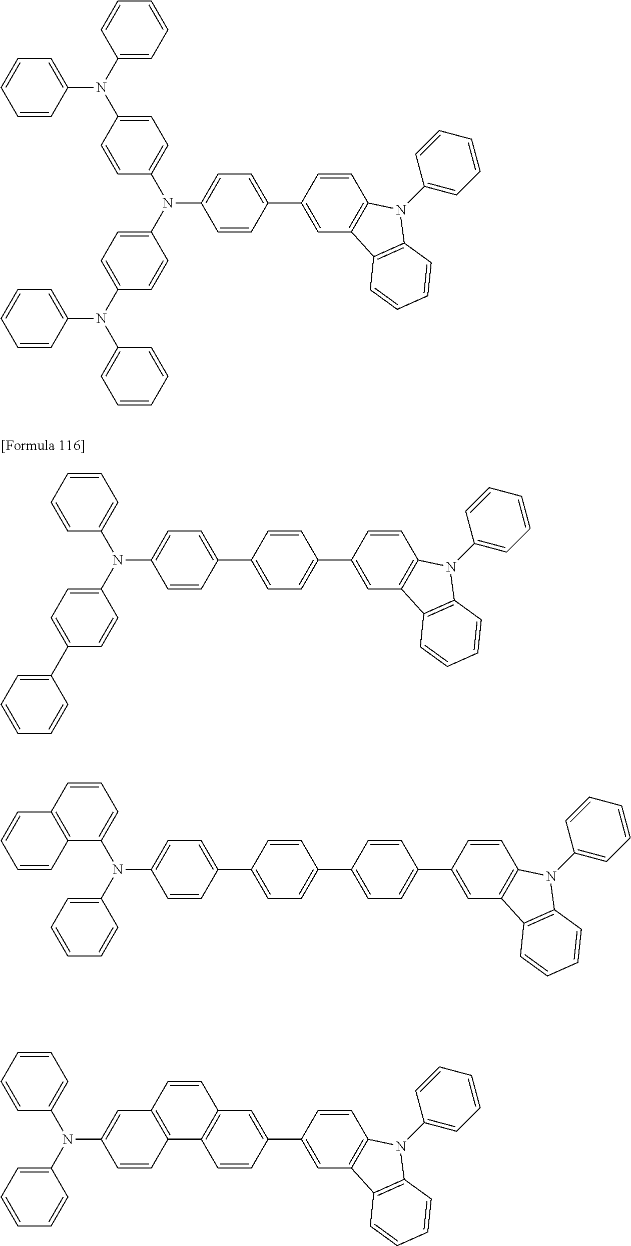 [SMILES: c1ccc(N(c2ccccc2)c2ccc(N(c3ccc(-c4ccc5c(c4)c4ccccc4n5-c4ccccc4)cc3)c3ccc(N(c4ccccc4)c4ccccc4)cc3)cc2)cc1 ; c1ccc(-c2ccc(N(c3ccccc3)c3ccc(-c4ccc(-c5ccc6c(c5)c5ccccc5n6-c5ccccc5)cc4)cc3)cc2)cc1 ; c1ccc(N(c2ccc(-c3ccc(-c4ccc(-c5ccc6c(c5)c5ccccc5n6-c5ccccc5)cc4)cc3)cc2)c2cccc3ccccc23)cc1 ; c1ccc(N(c2ccccc2)c2ccc3c(ccc4cc(-c5ccc6c(c5)c5ccccc5n6-c5ccccc5)ccc43)c2)cc1]

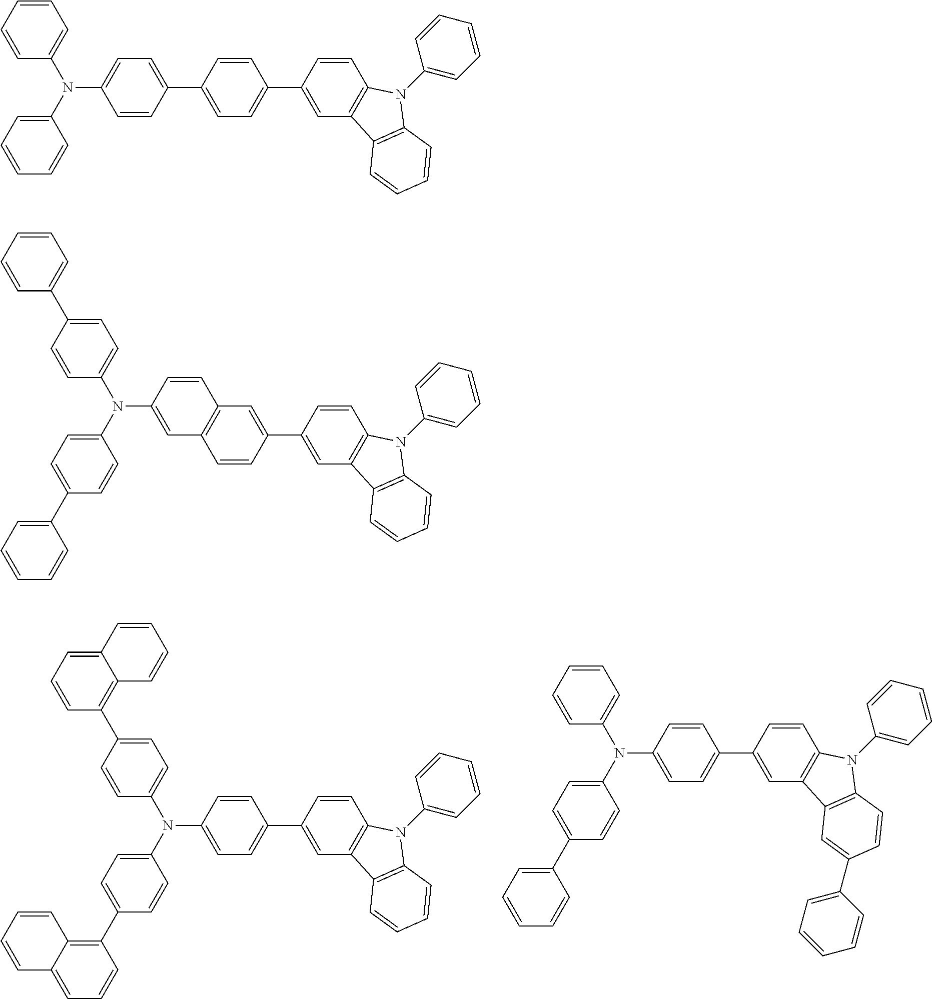 [SMILES: c1ccc(N(c2ccccc2)c2ccc(-c3ccc(-c4ccc5c(c4)c4ccccc4n5-c4ccccc4)cc3)cc2)cc1 ; c1ccc(-c2ccc(N(c3ccc(-c4ccccc4)cc3)c3ccc4cc(-c5ccc6c(c5)c5ccccc5n6-c5ccccc5)ccc4c3)cc2)cc1 ; c1ccc(-n2c3ccccc3c3cc(-c4ccc(N(c5ccc(-c6cccc7ccccc67)cc5)c5ccc(-c6cccc7ccccc67)cc5)cc4)ccc32)cc1 ; c1ccc(-c2ccc(N(c3ccccc3)c3ccc(-c4ccc5c(c4)c4cc(-c6ccccc6)ccc4n5-c4ccccc4)cc3)cc2)cc1]

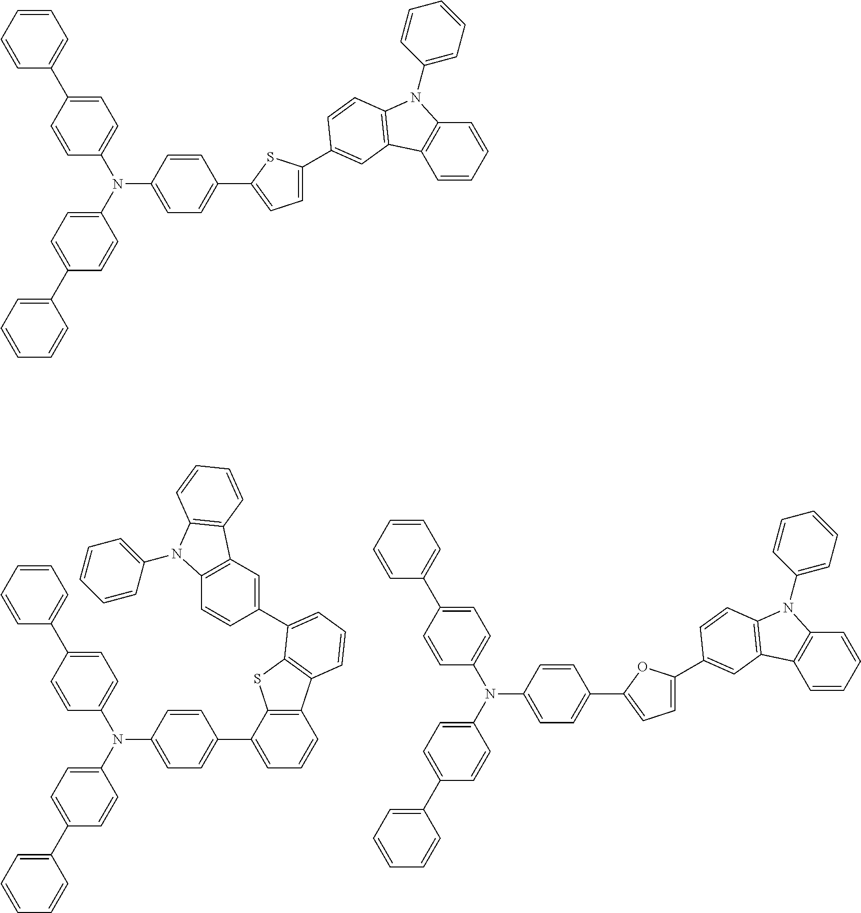 [SMILES: c1ccc(-c2ccc(N(c3ccc(-c4ccccc4)cc3)c3ccc(-c4ccc(-c5ccc6c(c5)c5ccccc5n6-c5ccccc5)s4)cc3)cc2)cc1 ; c1ccc(-c2ccc(N(c3ccc(-c4ccccc4)cc3)c3ccc(-c4cccc5c4sc4c(-c6ccc7c(c6)c6ccccc6n7-c6ccccc6)cccc45)cc3)cc2)cc1 ; c1ccc(-c2ccc(N(c3ccc(-c4ccccc4)cc3)c3ccc(-c4ccc(-c5ccc6c(c5)c5ccccc5n6-c5ccccc5)o4)cc3)cc2)cc1]

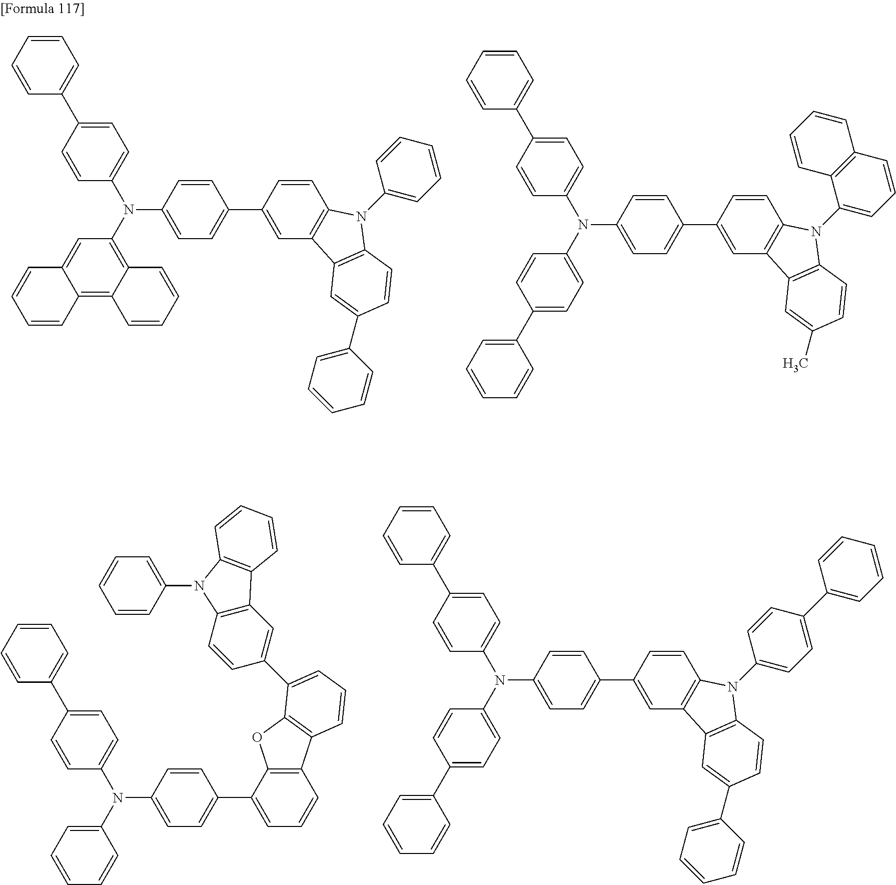 [SMILES: c1ccc(-c2ccc(N(c3ccc(-c4ccc5c(c4)c4cc(-c6ccccc6)ccc4n5-c4ccccc4)cc3)c3cc4ccccc4c4ccccc34)cc2)cc1 ; Cc1ccc2c(c1)c1cc(-c3ccc(N(c4ccc(-c5ccccc5)cc4)c4ccc(-c5ccccc5)cc4)cc3)ccc1n2-c1cccc2ccccc12 ; c1ccc(-c2ccc(N(c3ccccc3)c3ccc(-c4cccc5c4oc4c(-c6ccc7c(c6)c6ccccc6n7-c6ccccc6)cccc45)cc3)cc2)cc1 ; c1ccc(-c2ccc(N(c3ccc(-c4ccccc4)cc3)c3ccc(-c4ccc5c(c4)c4cc(-c6ccccc6)ccc4n5-c4ccc(-c5ccccc5)cc4)cc3)cc2)cc1]

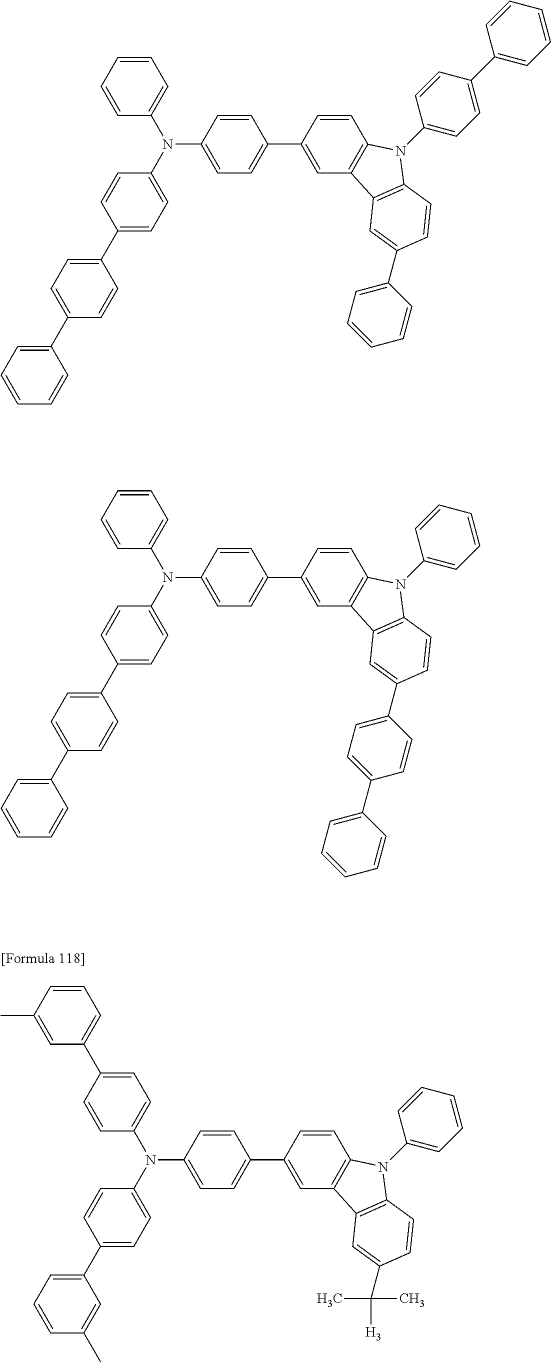

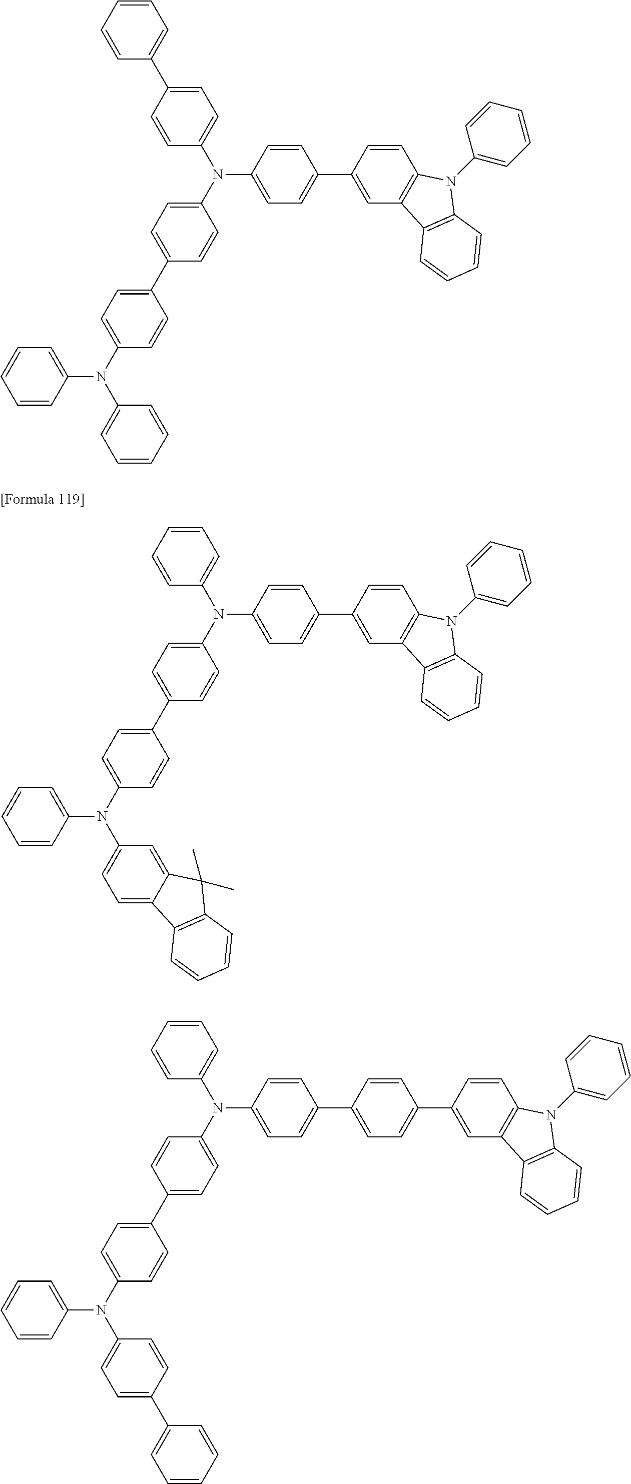

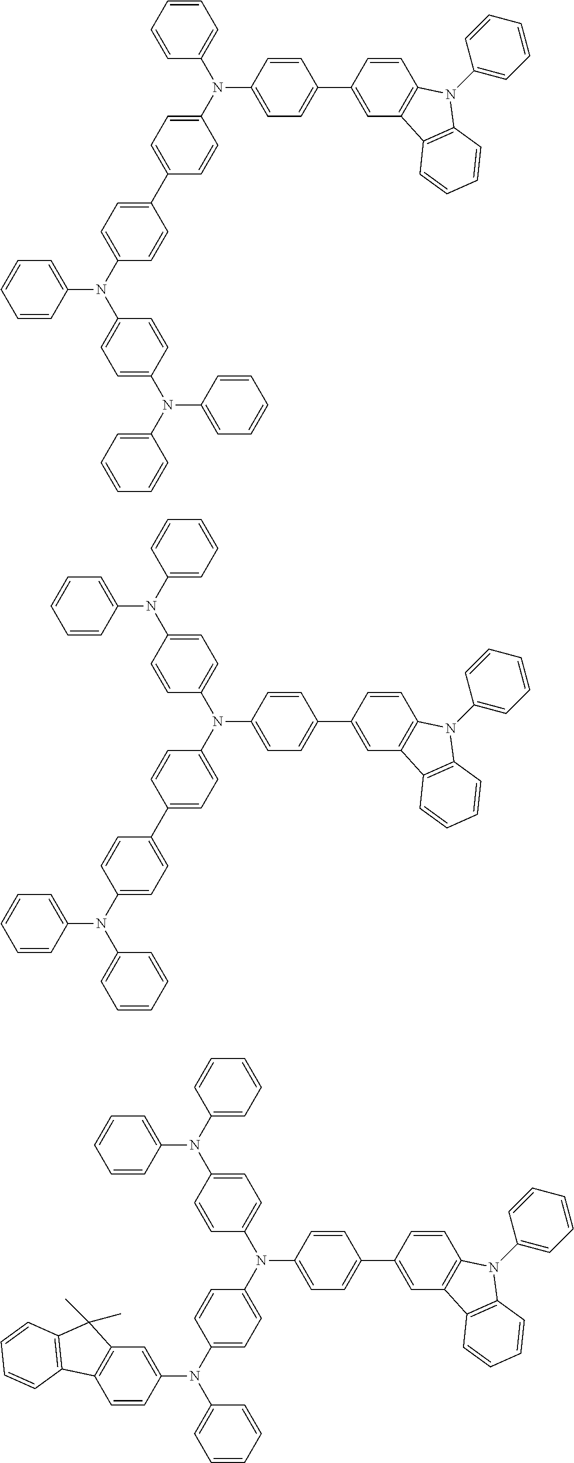

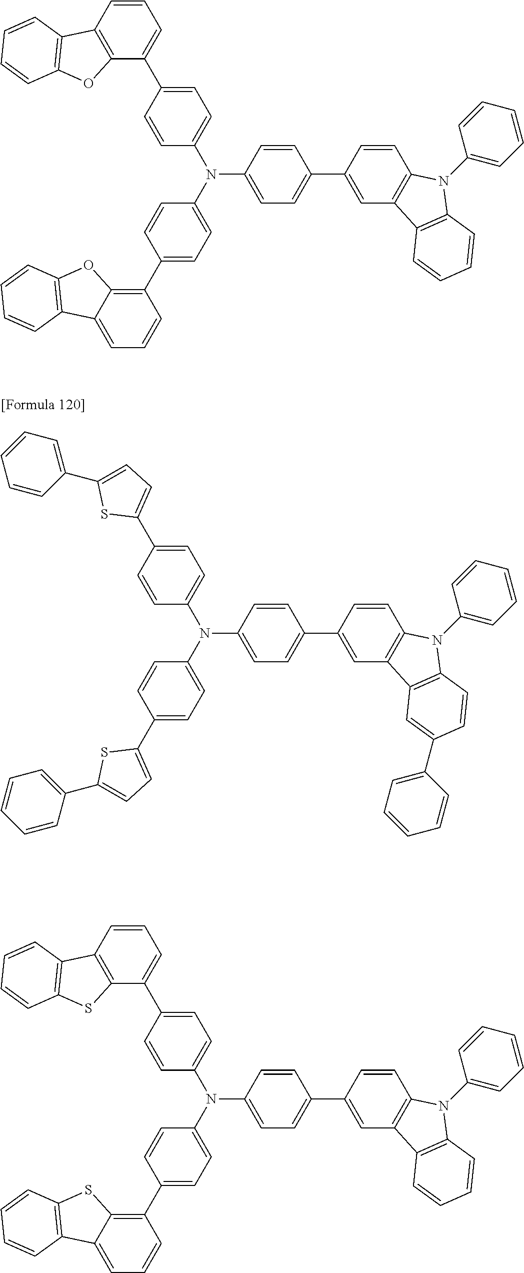 [SMILES: c1ccc(-n2c3ccccc3c3cc(-c4ccc(N(c5ccc(-c6cccc7c6oc6ccccc67)cc5)c5ccc(-c6cccc7c6oc6ccccc67)cc5)cc4)ccc32)cc1 ; c1ccc(-c2ccc3c(c2)c2cc(-c4ccc(N(c5ccc(-c6ccc(-c7ccccc7)s6)cc5)c5ccc(-c6ccc(-c7ccccc7)s6)cc5)cc4)ccc2n3-c2ccccc2)cc1 ; c1ccc(-n2c3ccccc3c3cc(-c4ccc(N(c5ccc(-c6cccc7c6sc6ccccc67)cc5)c5ccc(-c6cccc7c6sc6ccccc67)cc5)cc4)ccc32)cc1]

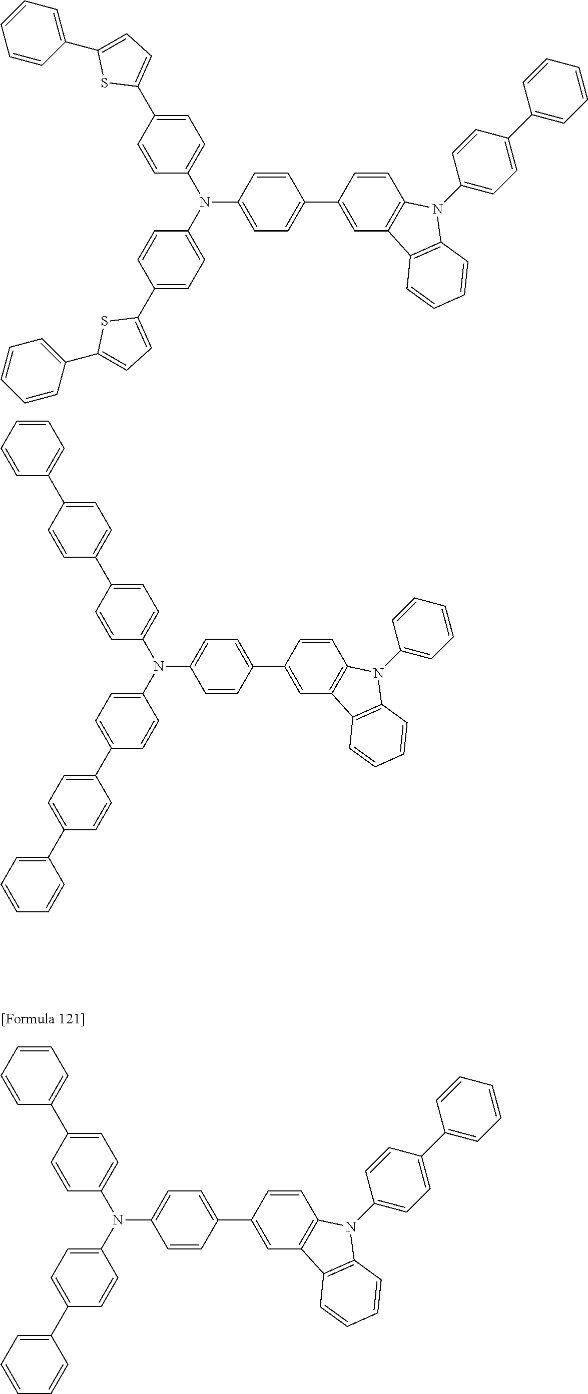

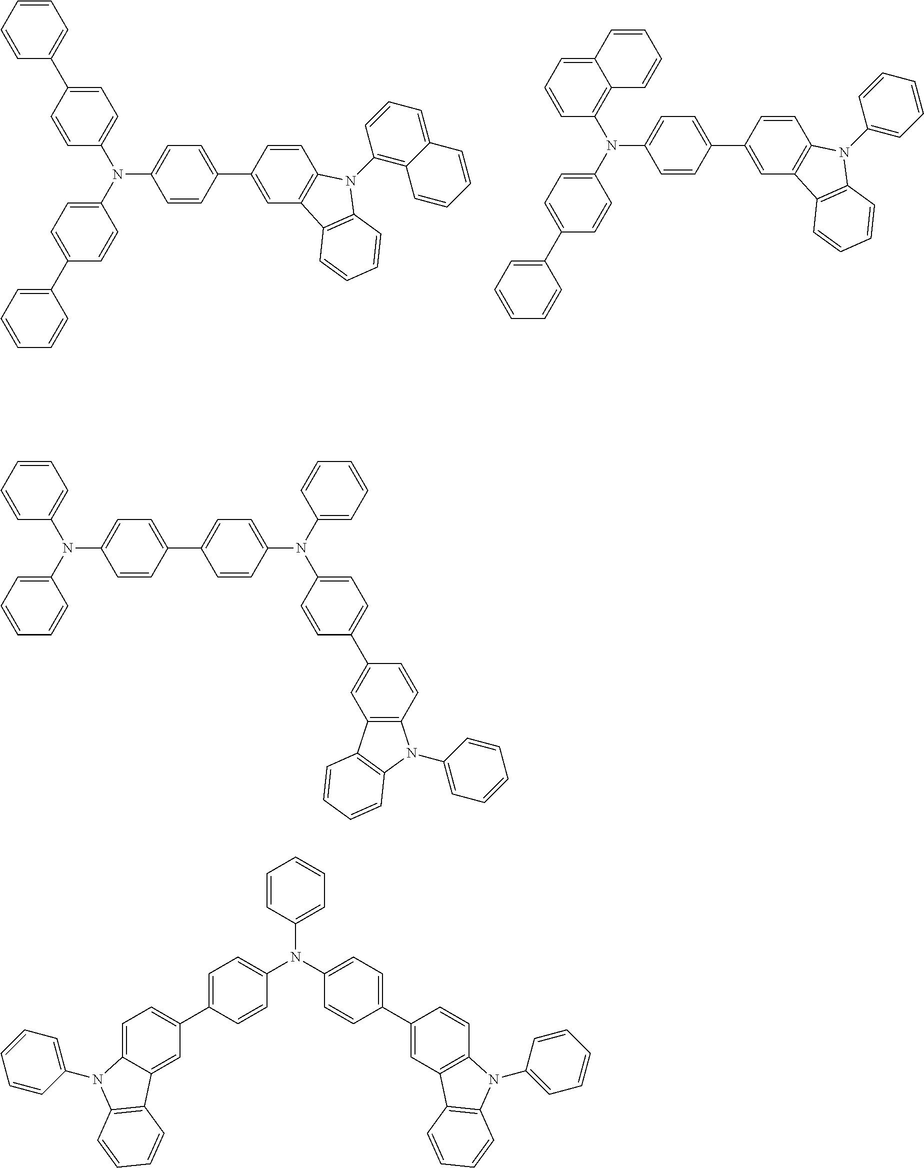 [SMILES: c1ccc(-c2ccc(N(c3ccc(-c4ccccc4)cc3)c3ccc(-c4ccc5c(c4)c4ccccc4n5-c4cccc5ccccc45)cc3)cc2)cc1 ; c1ccc(-c2ccc(N(c3ccc(-c4ccc5c(c4)c4ccccc4n5-c4ccccc4)cc3)c3cccc4ccccc34)cc2)cc1 ; c1ccc(N(c2ccccc2)c2ccc(-c3ccc(N(c4ccccc4)c4ccc(-c5ccc6c(c5)c5ccccc5n6-c5ccccc5)cc4)cc3)cc2)cc1 ; c1ccc(N(c2ccc(-c3ccc4c(c3)c3ccccc3n4-c3ccccc3)cc2)c2ccc(-c3ccc4c(c3)c3ccccc3n4-c3ccccc3)cc2)cc1]

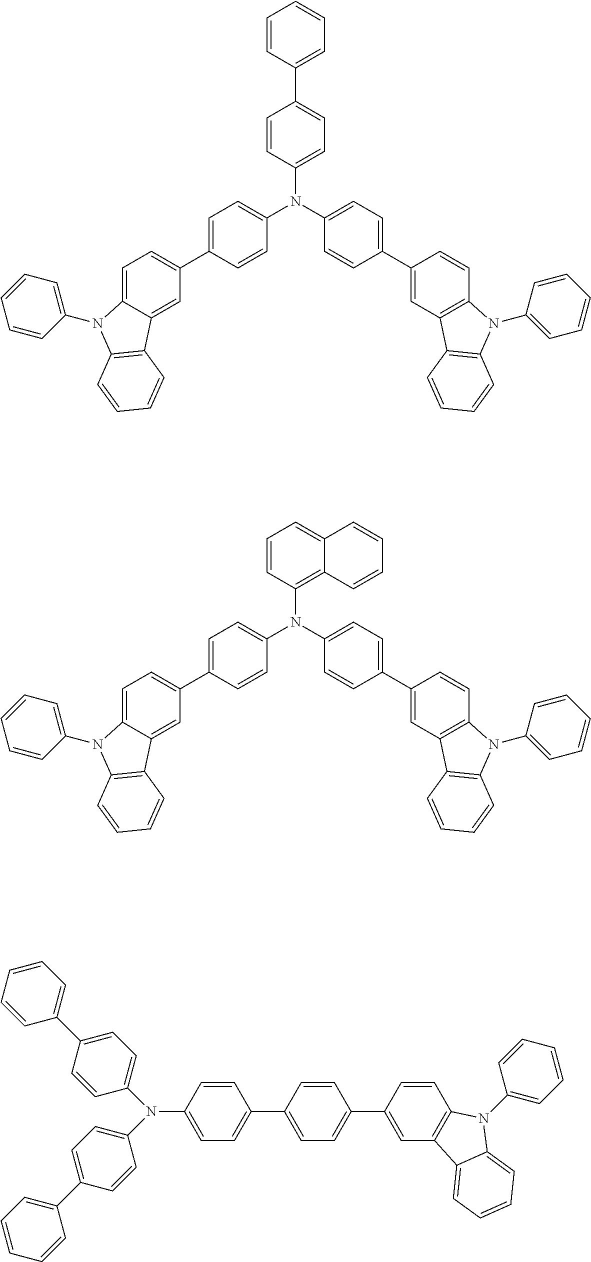 [SMILES: c1ccc(-c2ccc(N(c3ccc(-c4ccc5c(c4)c4ccccc4n5-c4ccccc4)cc3)c3ccc(-c4ccc5c(c4)c4ccccc4n5-c4ccccc4)cc3)cc2)cc1 ; c1ccc(-n2c3ccccc3c3cc(-c4ccc(N(c5ccc(-c6ccc7c(c6)c6ccccc6n7-c6ccccc6)cc5)c5cccc6ccccc56)cc4)ccc32)cc1 ; c1ccc(-c2ccc(N(c3ccc(-c4ccccc4)cc3)c3ccc(-c4ccc(-c5ccc6c(c5)c5ccccc5n6-c5ccccc5)cc4)cc3)cc2)cc1]

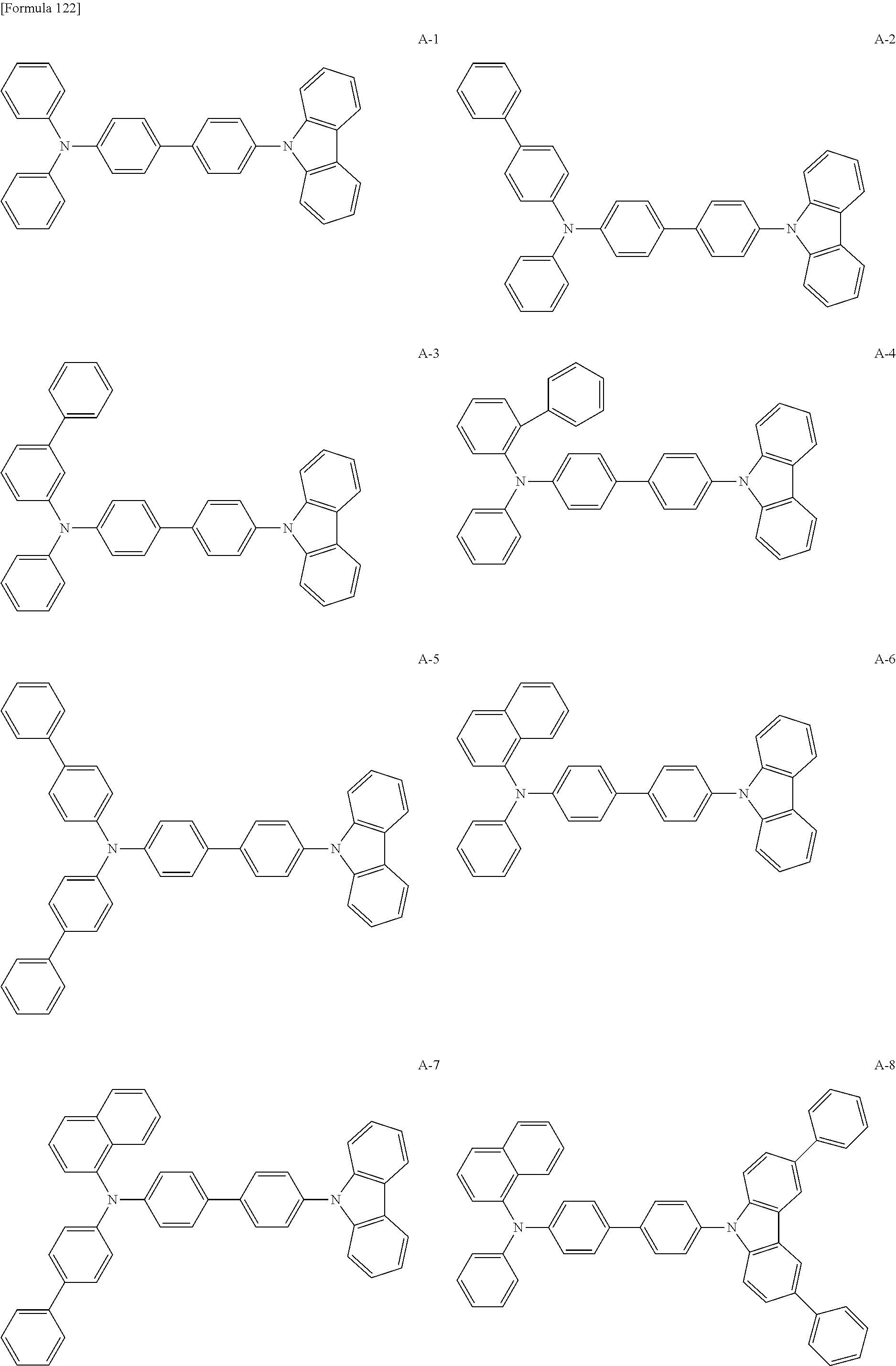

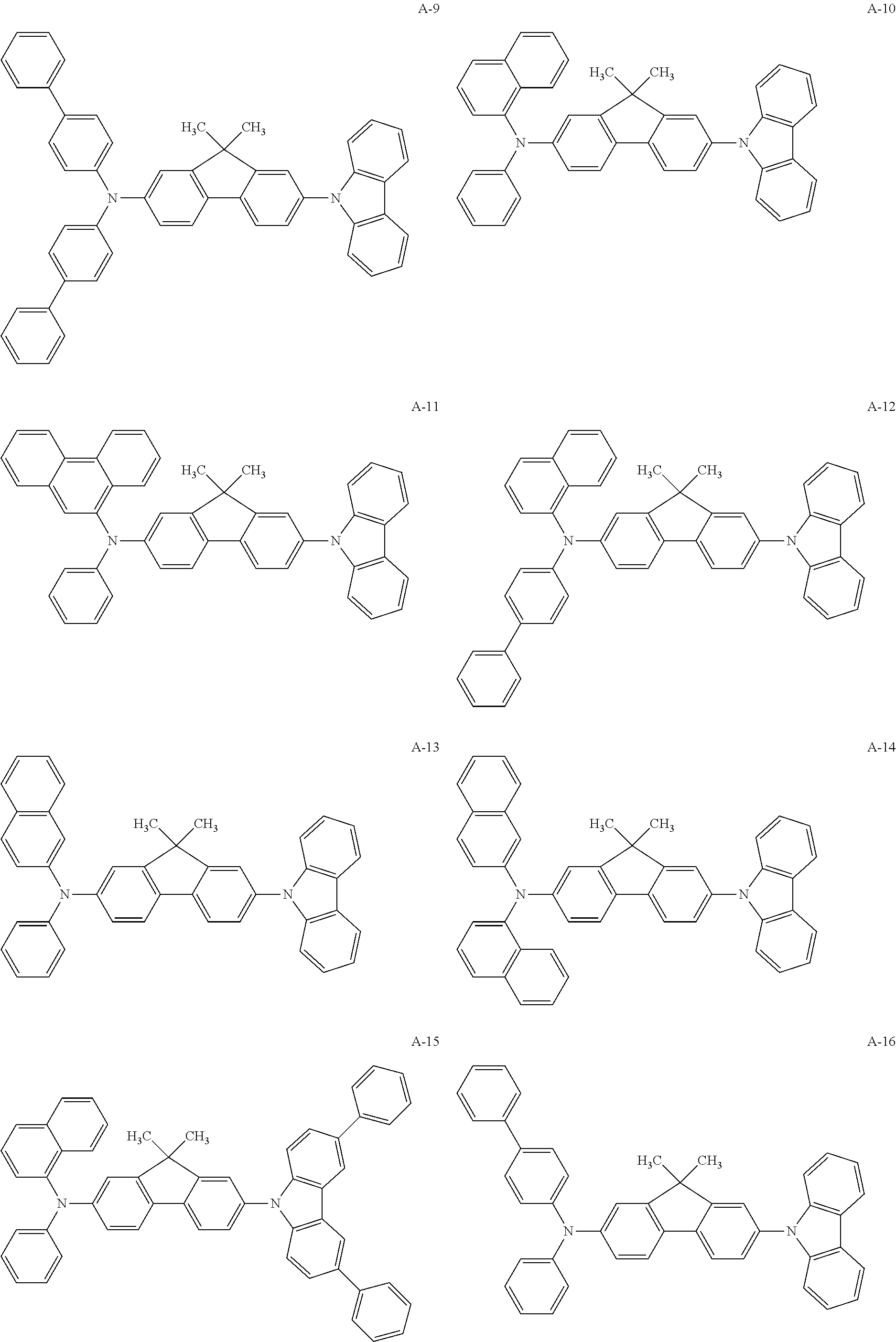

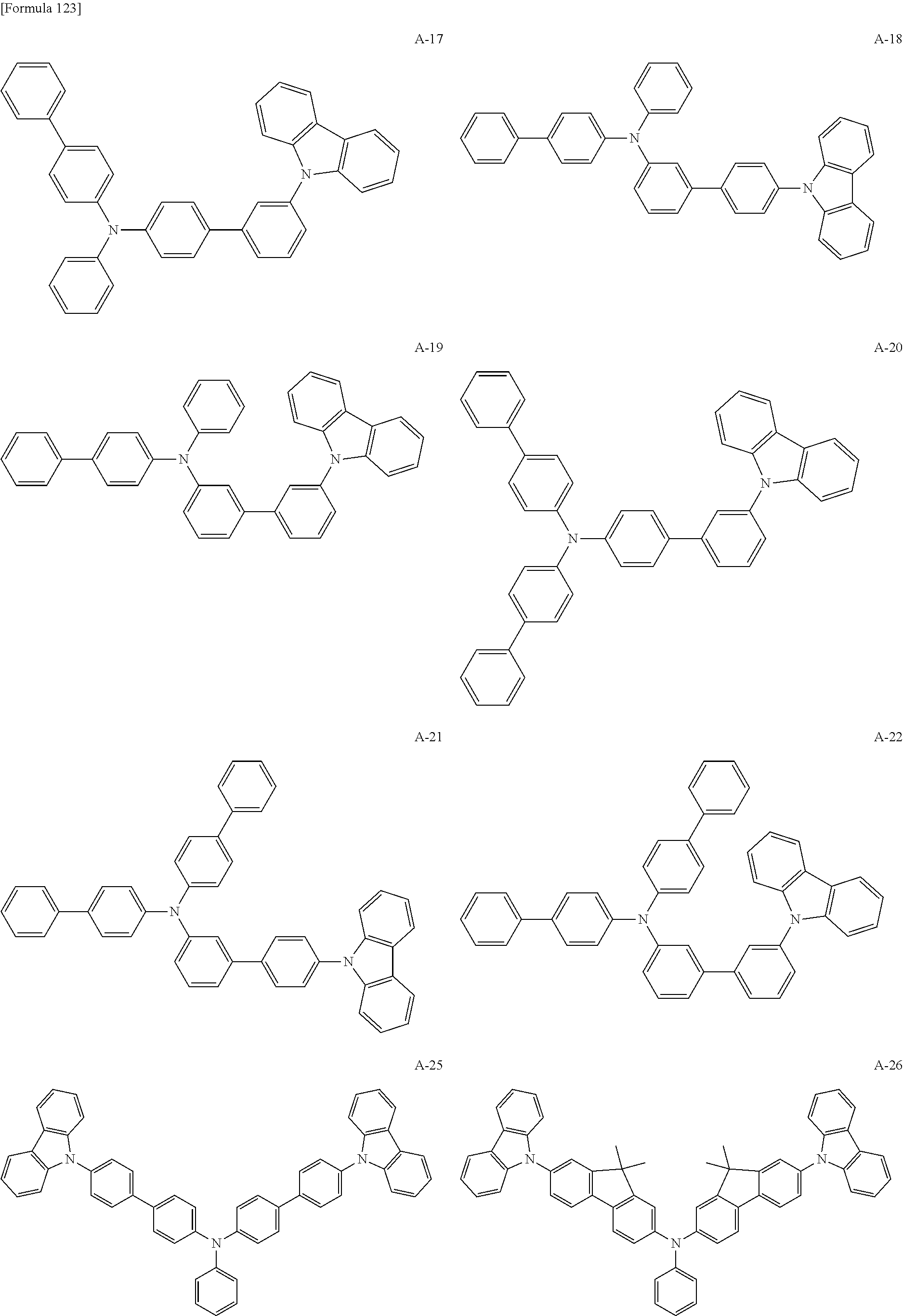

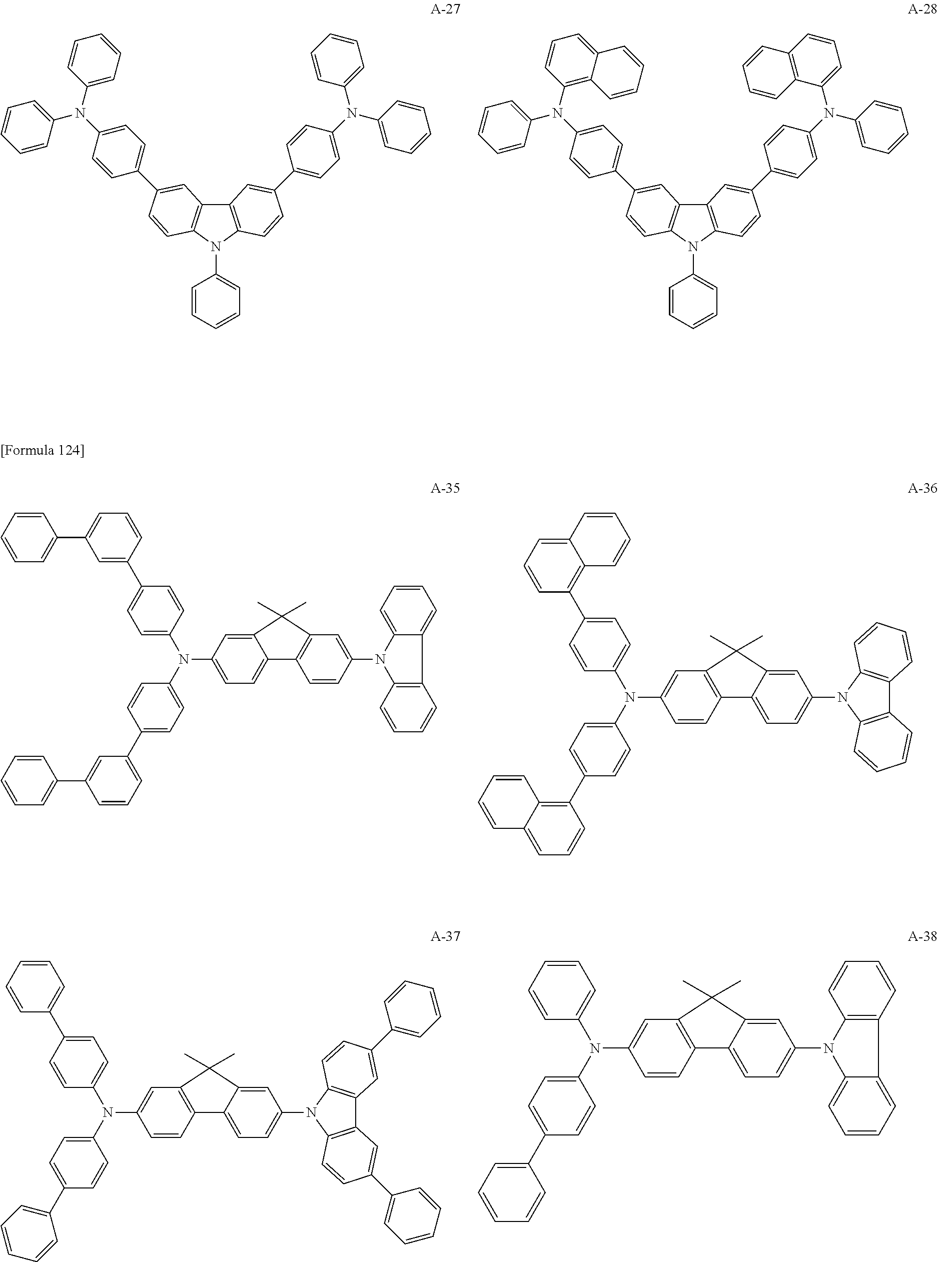

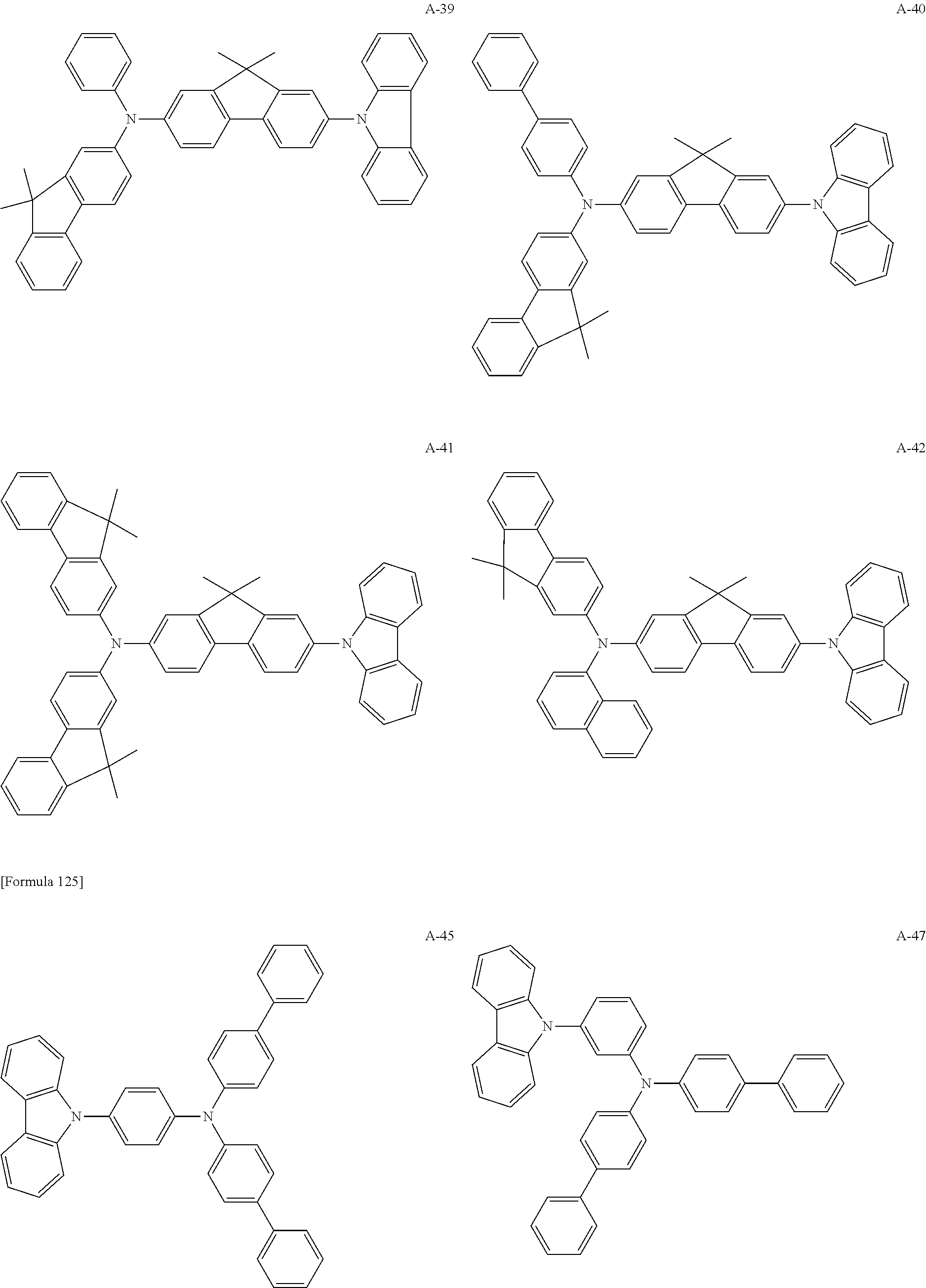

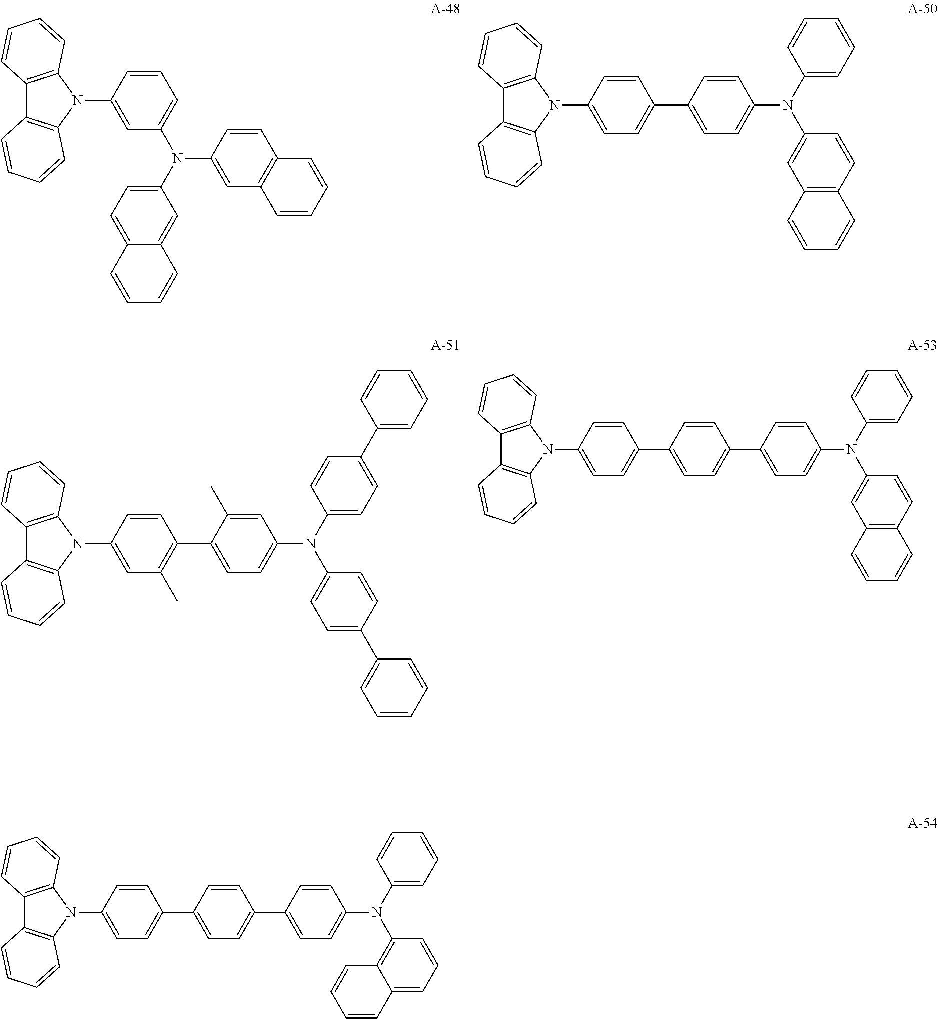 [SMILES: c1cc(N(c2ccc3ccccc3c2)c2ccc3ccccc3c2)cc(-n2c3ccccc3c3ccccc32)c1 ; c1ccc(N(c2ccc(-c3ccc(-n4c5ccccc5c5ccccc54)cc3)cc2)c2ccc3ccccc3c2)cc1 ; Cc1cc(N(c2ccc(-c3ccccc3)cc2)c2ccc(-c3ccccc3)cc2)ccc1-c1ccc(-n2c3ccccc3c3ccccc32)cc1C ; c1ccc(N(c2ccc(-c3ccc(-c4ccc(-n5c6ccccc6c6ccccc65)cc4)cc3)cc2)c2ccc3ccccc3c2)cc1 ; c1ccc(N(c2ccc(-c3ccc(-c4ccc(-n5c6ccccc6c6ccccc65)cc4)cc3)cc2)c2cccc3ccccc23)cc1]

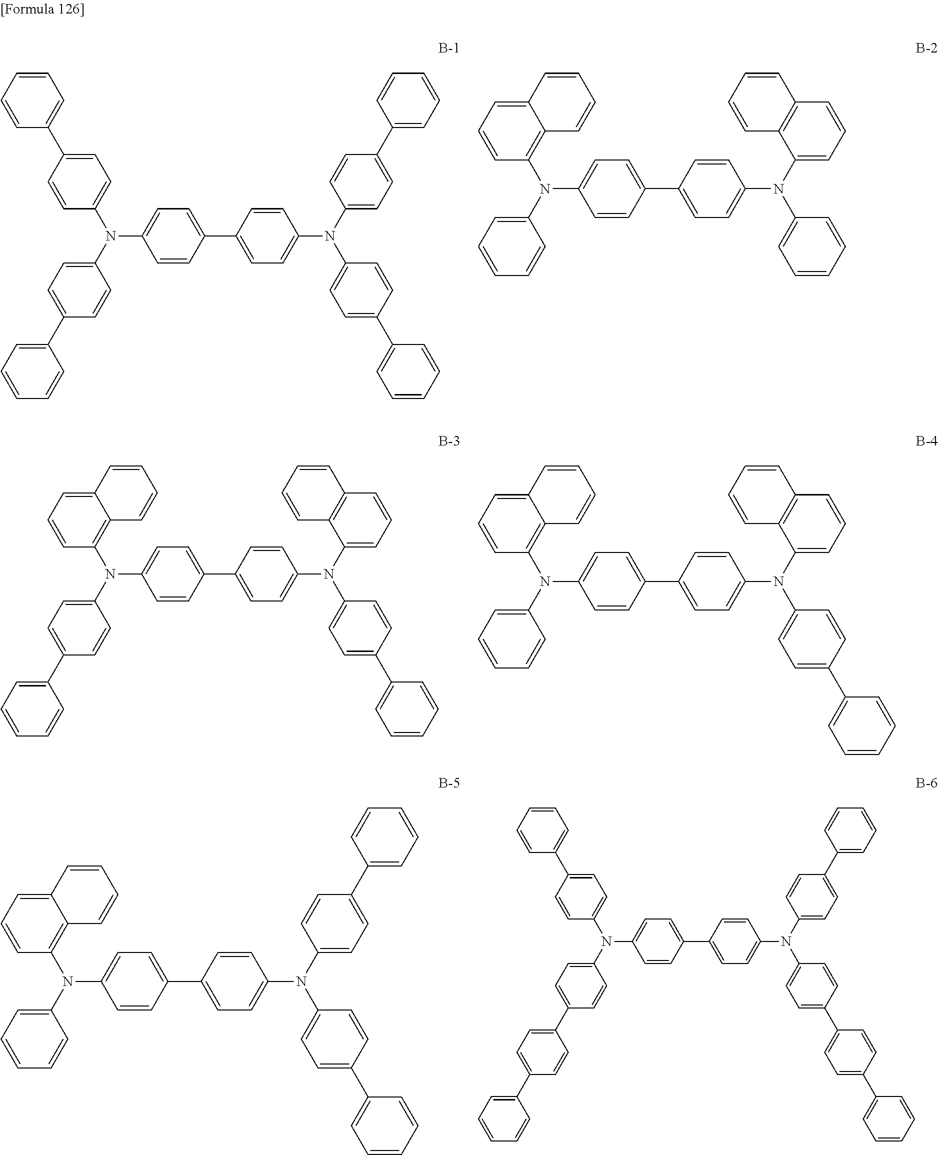

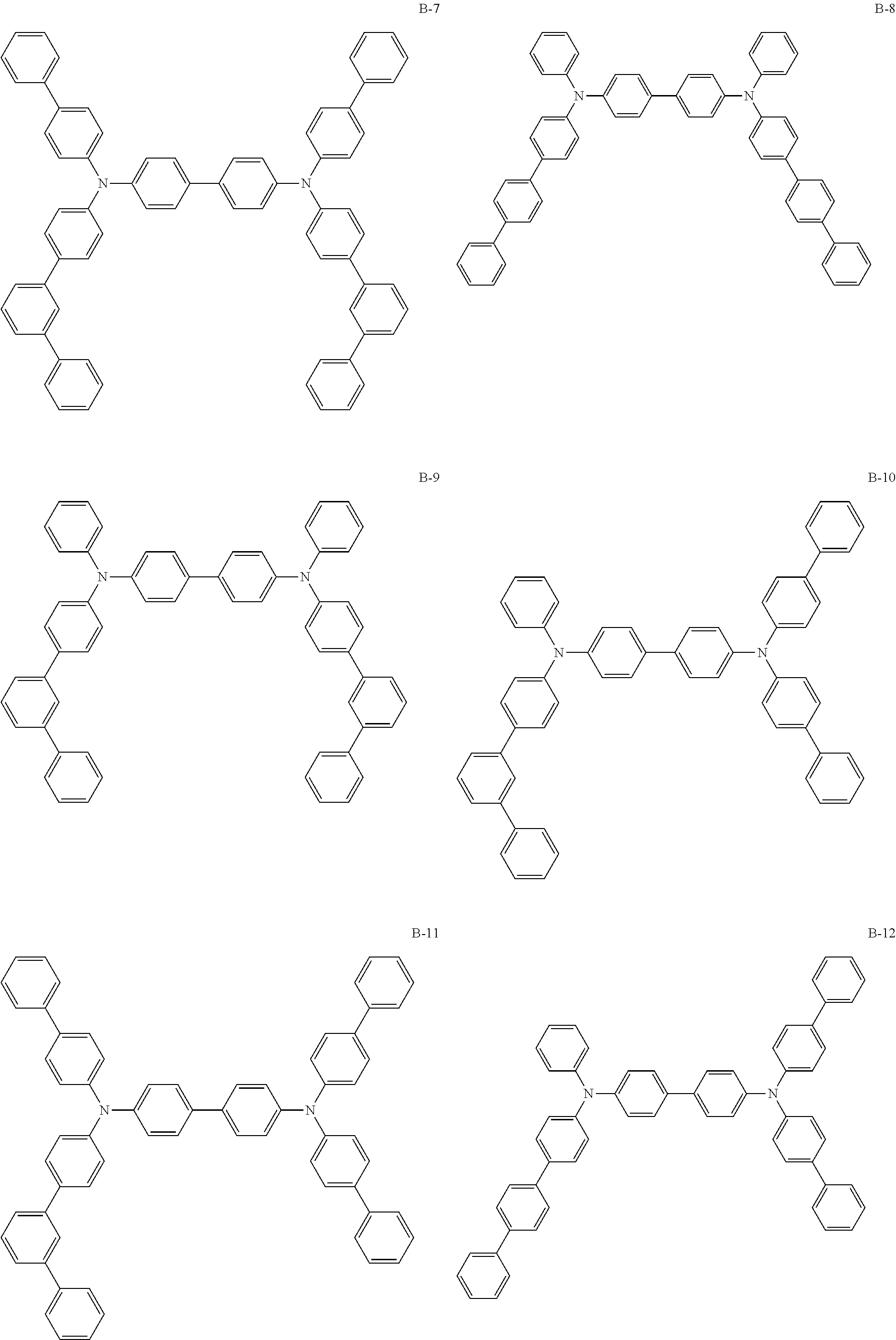

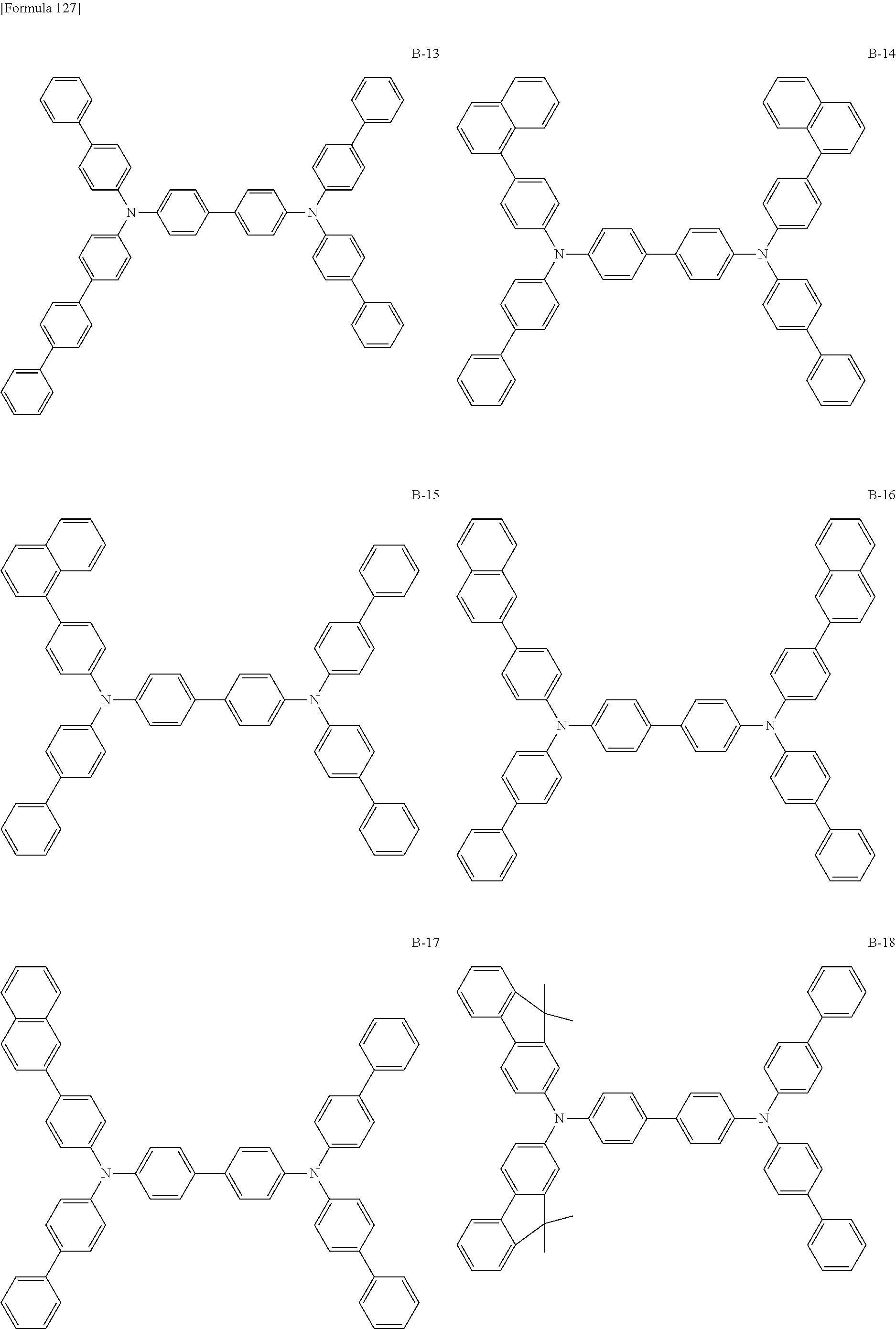

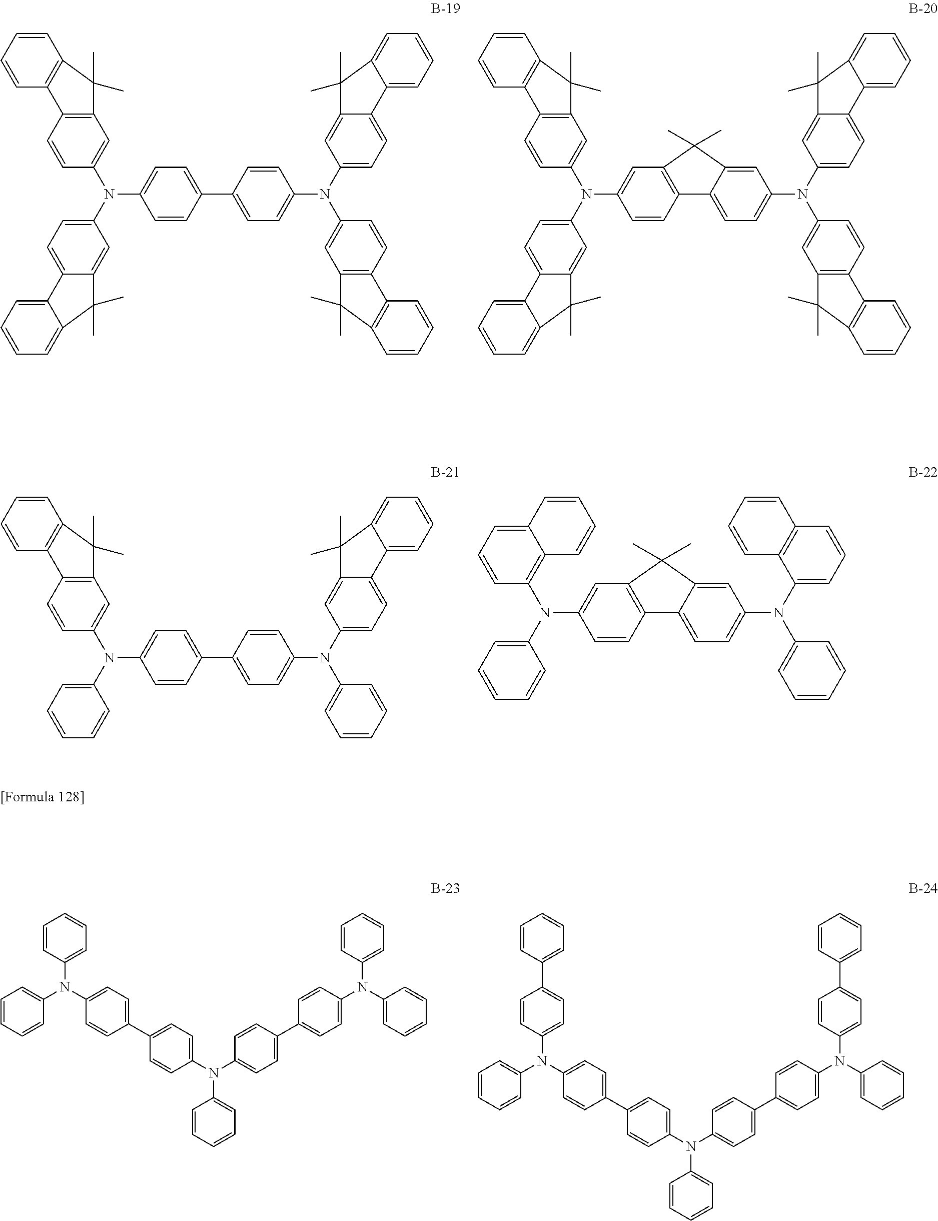

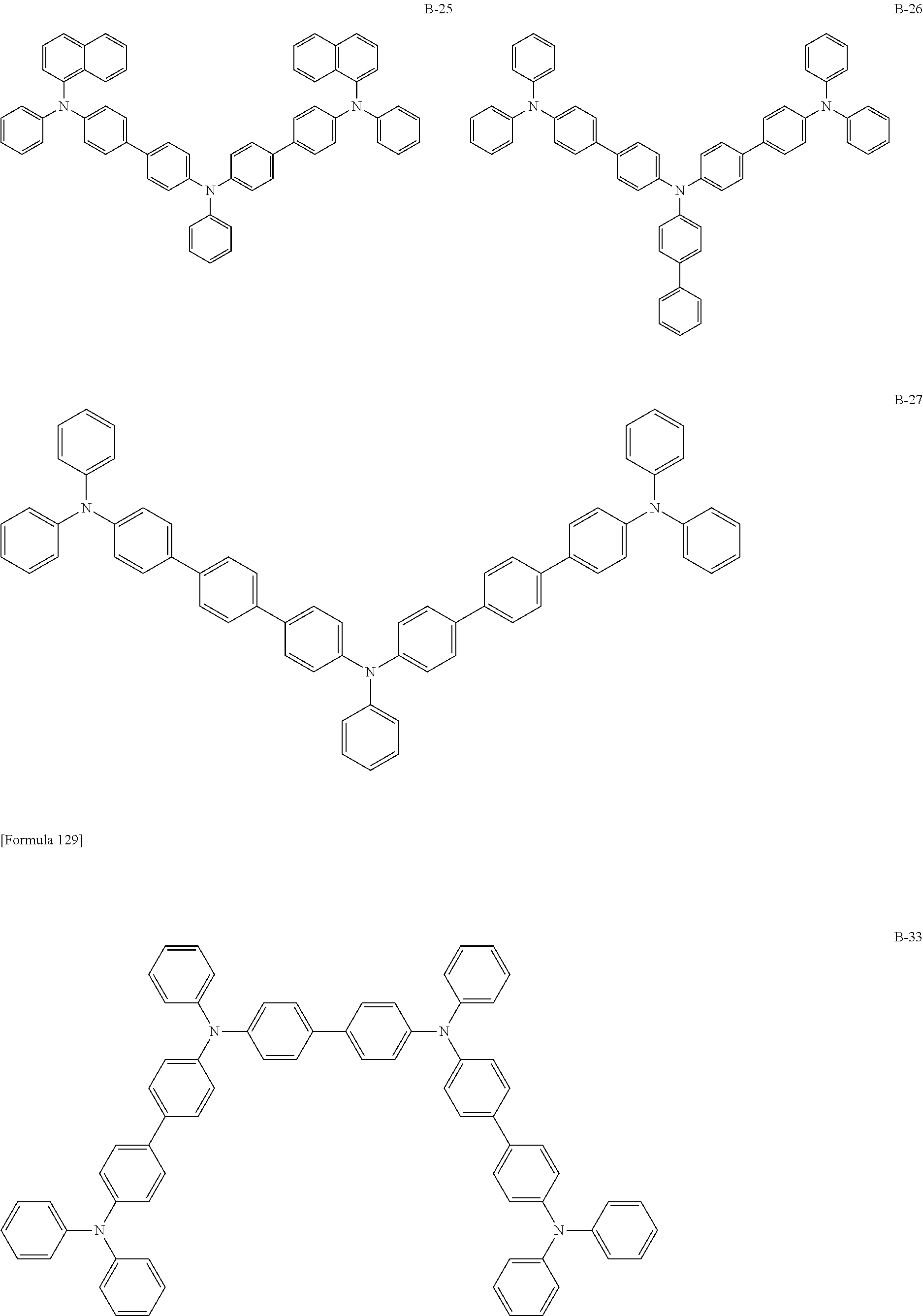 [SMILES: c1ccc(N(c2ccc(-c3ccc(N(c4ccccc4)c4cccc5ccccc45)cc3)cc2)c2ccc(-c3ccc(N(c4ccccc4)c4cccc5ccccc45)cc3)cc2)cc1 ; c1ccc(-c2ccc(N(c3ccc(-c4ccc(N(c5ccccc5)c5ccccc5)cc4)cc3)c3ccc(-c4ccc(N(c5ccccc5)c5ccccc5)cc4)cc3)cc2)cc1 ; c1ccc(N(c2ccccc2)c2ccc(-c3ccc(-c4ccc(N(c5ccccc5)c5ccc(-c6ccc(-c7ccc(N(c8ccccc8)c8ccccc8)cc7)cc6)cc5)cc4)cc3)cc2)cc1 ; c1ccc(N(c2ccccc2)c2ccc(-c3ccc(N(c4ccccc4)c4ccc(-c5ccc(N(c6ccccc6)c6ccc(-c7ccc(N(c8ccccc8)c8ccccc8)cc7)cc6)cc5)cc4)cc3)cc2)cc1]

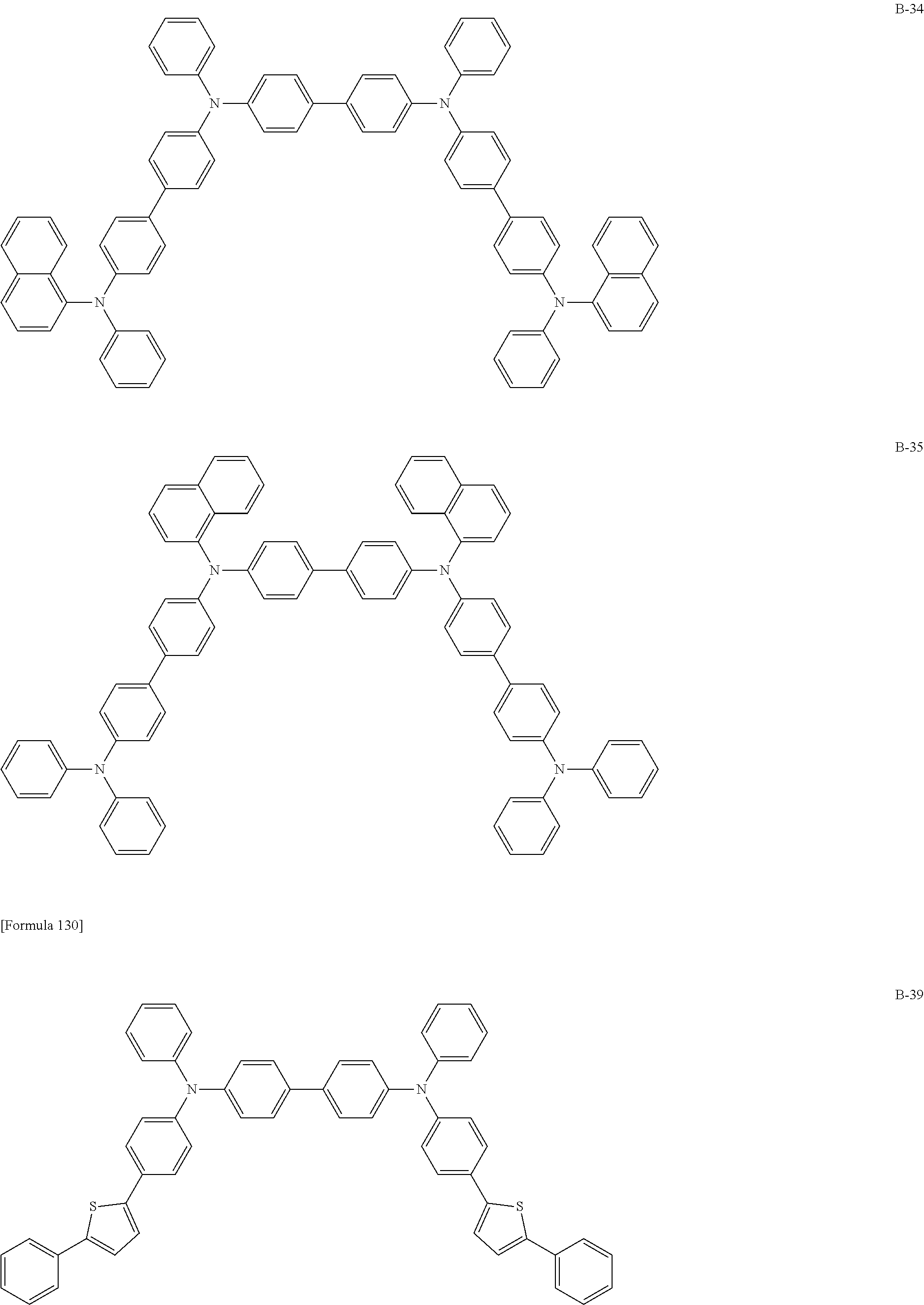 [SMILES: c1ccc(N(c2ccc(-c3ccc(N(c4ccccc4)c4ccc(-c5ccc(N(c6ccccc6)c6cccc7ccccc67)cc5)cc4)cc3)cc2)c2ccc(-c3ccc(N(c4ccccc4)c4cccc5ccccc45)cc3)cc2)cc1 ; c1ccc(N(c2ccccc2)c2ccc(-c3ccc(N(c4ccc(-c5ccc(N(c6ccc(-c7ccc(N(c8ccccc8)c8ccccc8)cc7)cc6)c6cccc7ccccc67)cc5)cc4)c4cccc5ccccc45)cc3)cc2)cc1 ; c1ccc(-c2ccc(-c3ccc(N(c4ccccc4)c4ccc(-c5ccc(N(c6ccccc6)c6ccc(-c7ccc(-c8ccccc8)s7)cc6)cc5)cc4)cc3)s2)cc1]

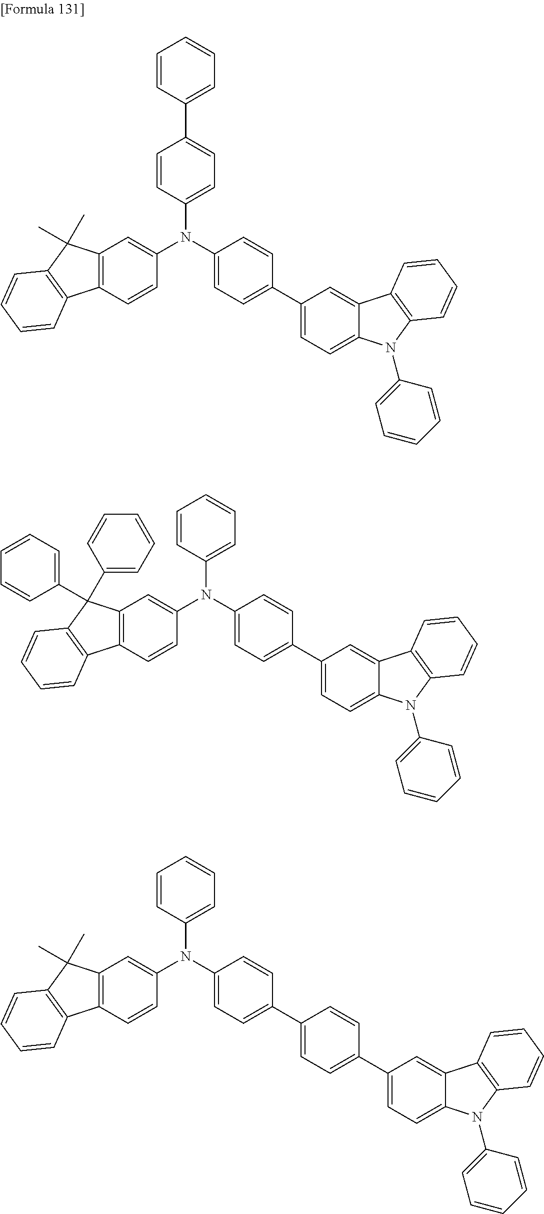 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1 ; CC1(C)c2ccccc2-c2ccc(N(c3ccccc3)c3ccc(-c4ccc(-c5ccc6c(c5)c5ccccc5n6-c5ccccc5)cc4)cc3)cc21]

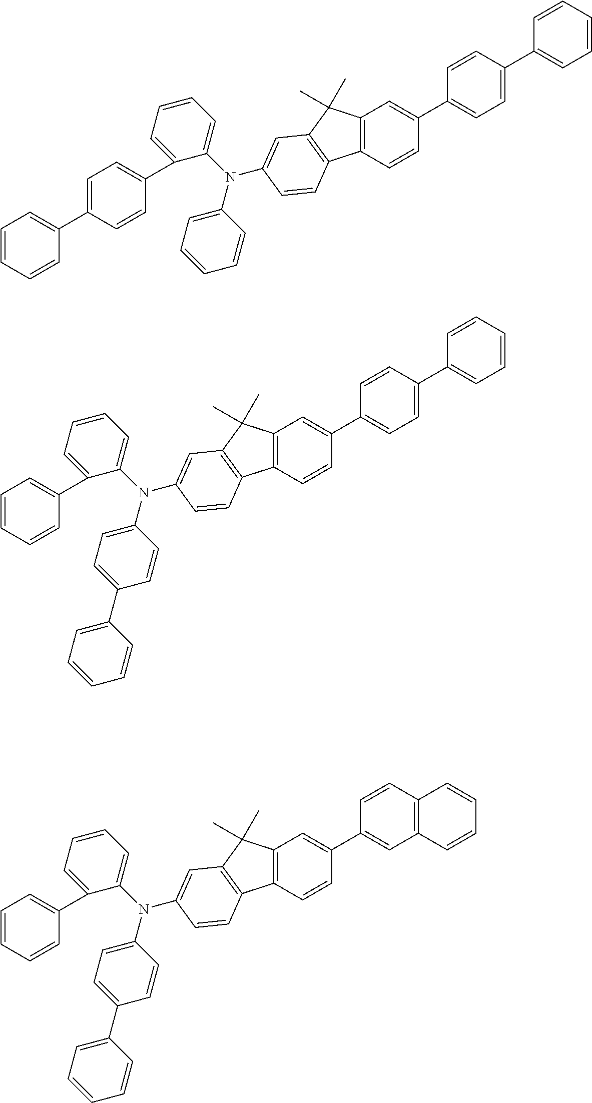 [SMILES: CC1(C)c2cc(-c3ccc(-c4ccccc4)cc3)ccc2-c2ccc(N(c3ccccc3)c3ccccc3-c3ccc(-c4ccccc4)cc3)cc21 ; CC1(C)c2cc(-c3ccc(-c4ccccc4)cc3)ccc2-c2ccc(N(c3ccc(-c4ccccc4)cc3)c3ccccc3-c3ccccc3)cc21 ; CC1(C)c2cc(-c3ccc4ccccc4c3)ccc2-c2ccc(N(c3ccc(-c4ccccc4)cc3)c3ccccc3-c3ccccc3)cc21]

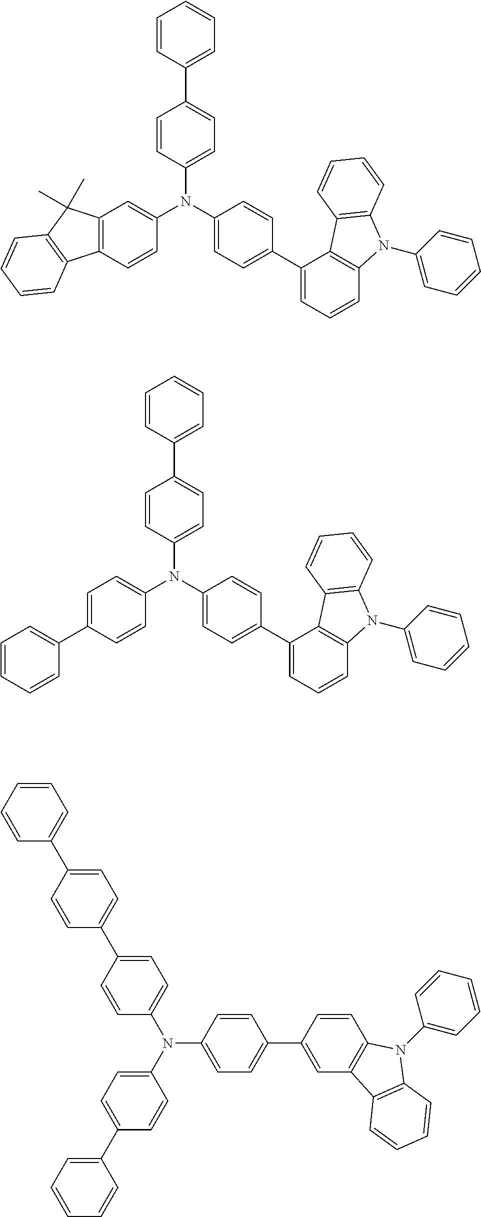 [SMILES: CC1(C)c2ccccc2-c2ccc(N(c3ccc(-c4ccccc4)cc3)c3ccc(-c4cccc5c4c4ccccc4n5-c4ccccc4)cc3)cc21 ; c1ccc(-c2ccc(N(c3ccc(-c4ccccc4)cc3)c3ccc(-c4cccc5c4c4ccccc4n5-c4ccccc4)cc3)cc2)cc1 ; c1ccc(-c2ccc(-c3ccc(N(c4ccc(-c5ccccc5)cc4)c4ccc(-c5ccc6c(c5)c5ccccc5n6-c5ccccc5)cc4)cc3)cc2)cc1]

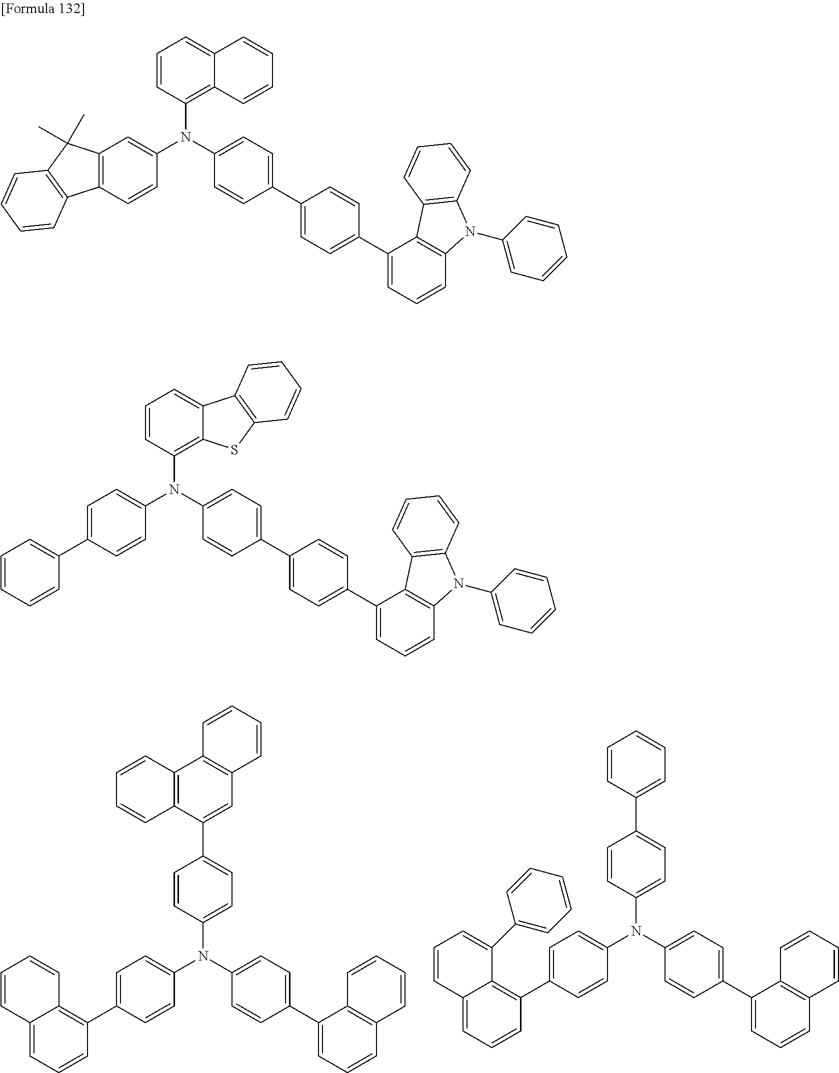

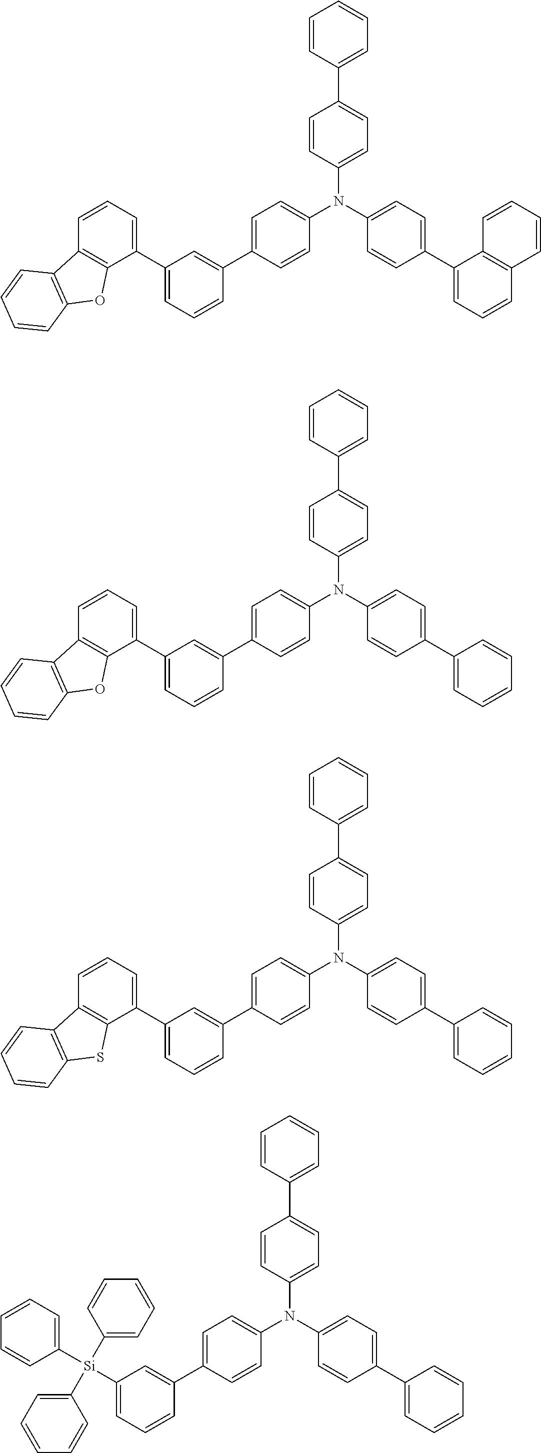 [SMILES: c1ccc(-c2ccc(N(c3ccc(-c4cccc(-c5cccc6c5oc5ccccc56)c4)cc3)c3ccc(-c4cccc5ccccc45)cc3)cc2)cc1 ; c1ccc(-c2ccc(N(c3ccc(-c4ccccc4)cc3)c3ccc(-c4cccc(-c5cccc6c5oc5ccccc56)c4)cc3)cc2)cc1 ; c1ccc(-c2ccc(N(c3ccc(-c4ccccc4)cc3)c3ccc(-c4cccc(-c5cccc6c5sc5ccccc56)c4)cc3)cc2)cc1 ; c1ccc(-c2ccc(N(c3ccc(-c4ccccc4)cc3)c3ccc(-c4cccc([Si](c5ccccc5)(c5ccccc5)c5ccccc5)c4)cc3)cc2)cc1]

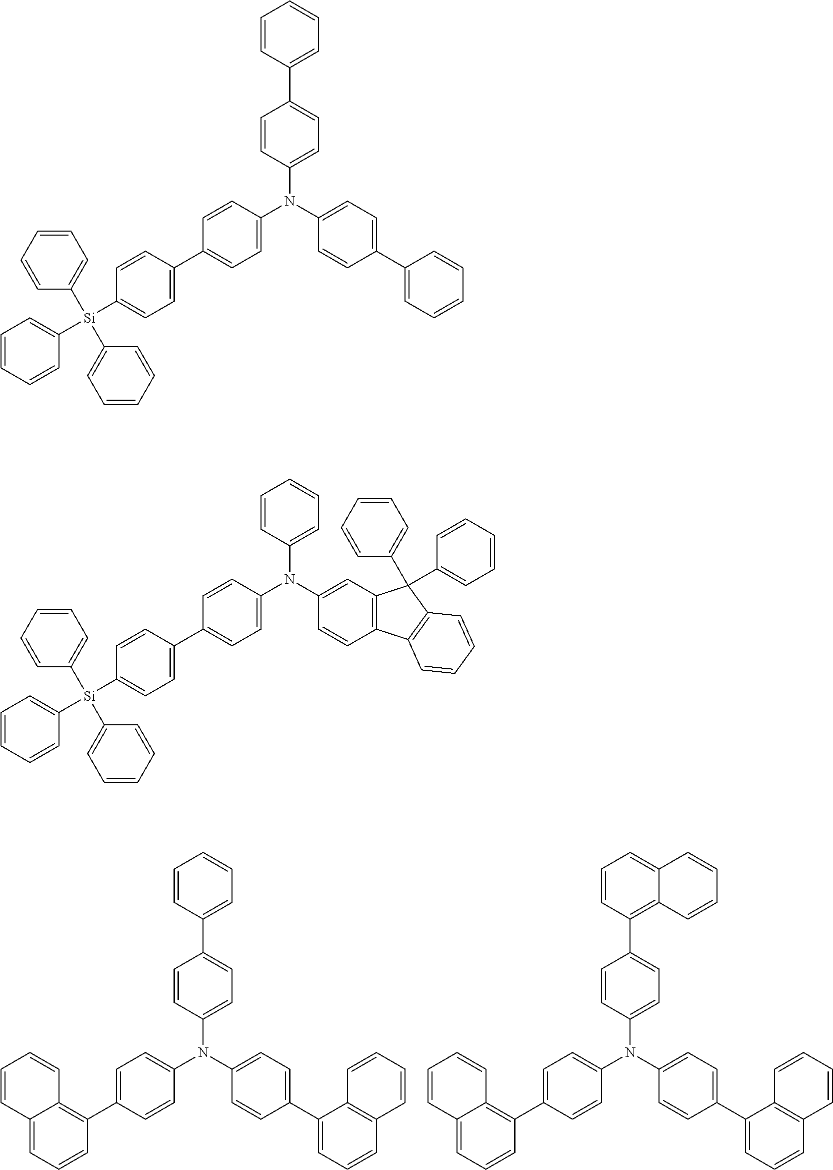

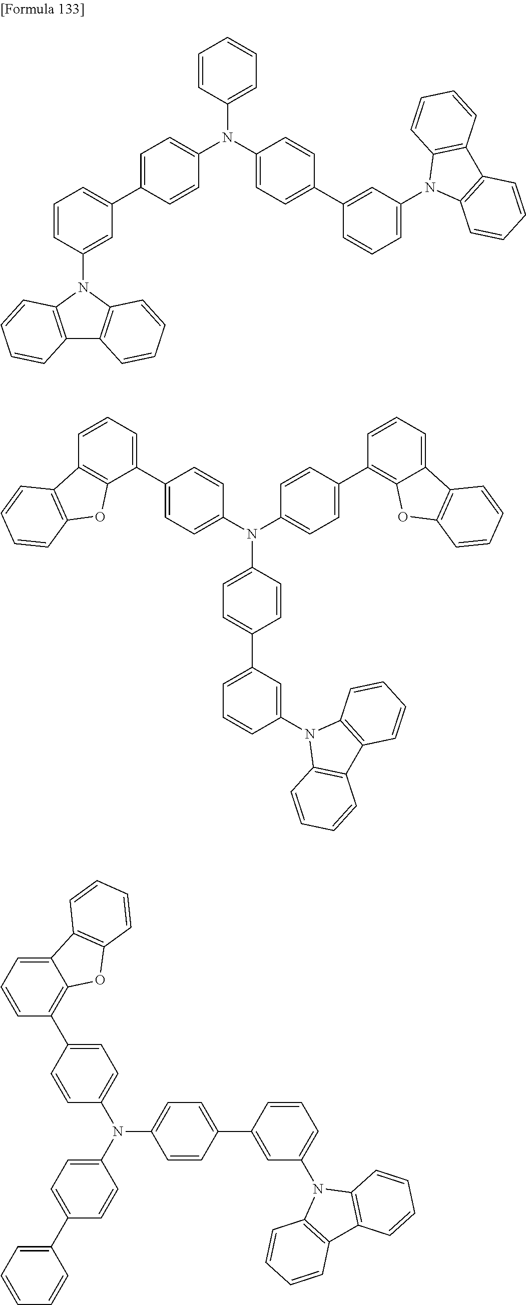 [SMILES: c1ccc(N(c2ccc(-c3cccc(-n4c5ccccc5c5ccccc54)c3)cc2)c2ccc(-c3cccc(-n4c5ccccc5c5ccccc54)c3)cc2)cc1 ; c1cc(-c2ccc(N(c3ccc(-c4cccc5c4oc4ccccc45)cc3)c3ccc(-c4cccc5c4oc4ccccc45)cc3)cc2)cc(-n2c3ccccc3c3ccccc32)c1 ; c1ccc(-c2ccc(N(c3ccc(-c4cccc(-n5c6ccccc6c6ccccc65)c4)cc3)c3ccc(-c4cccc5c4oc4ccccc45)cc3)cc2)cc1]

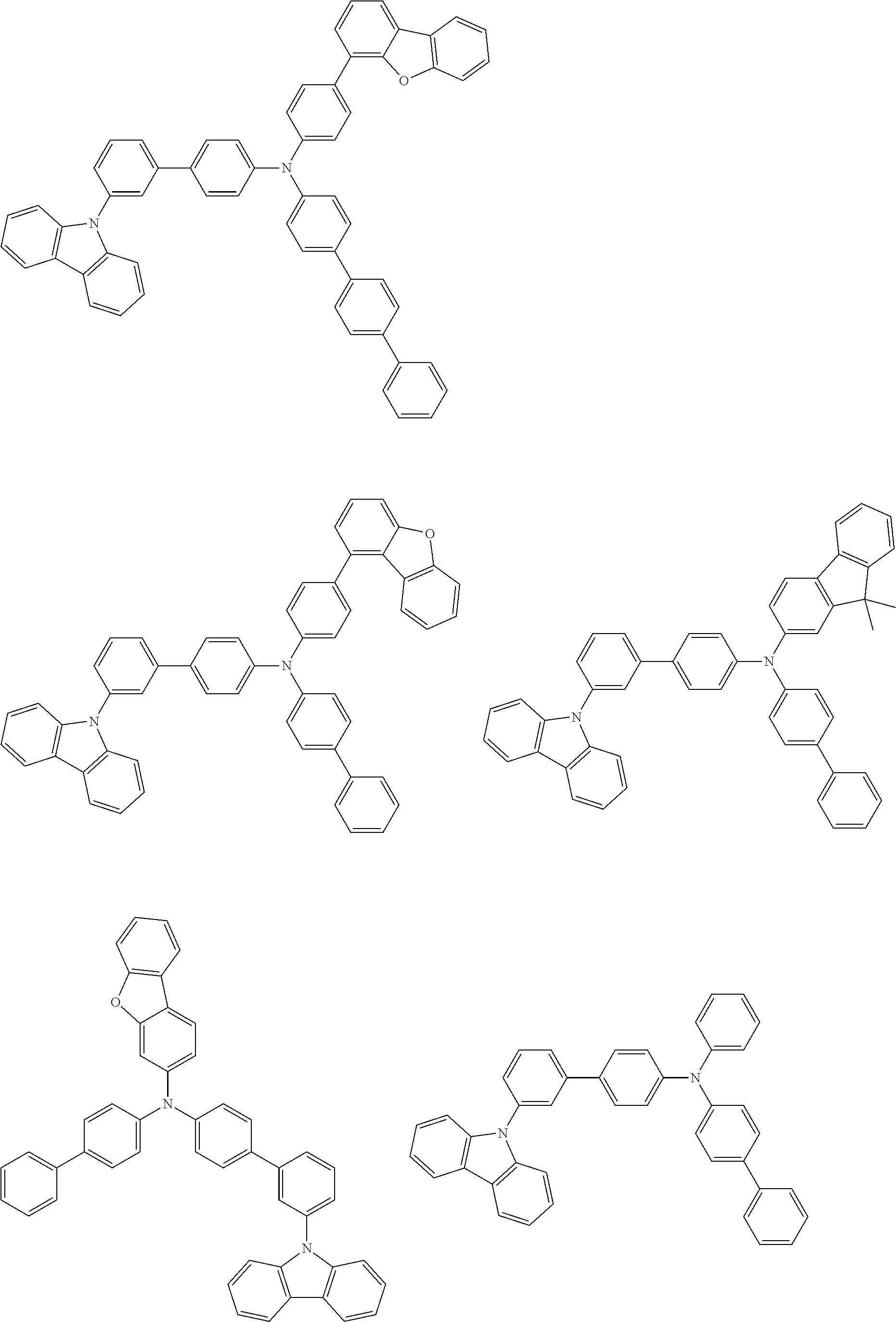 [SMILES: c1ccc(-c2ccc(-c3ccc(N(c4ccc(-c5cccc(-n6c7ccccc7c7ccccc76)c5)cc4)c4ccc(-c5cccc6c5oc5ccccc56)cc4)cc3)cc2)cc1 ; c1ccc(-c2ccc(N(c3ccc(-c4cccc(-n5c6ccccc6c6ccccc65)c4)cc3)c3ccc(-c4cccc5oc6ccccc6c45)cc3)cc2)cc1 ; CC1(C)c2ccccc2-c2ccc(N(c3ccc(-c4ccccc4)cc3)c3ccc(-c4cccc(-n5c6ccccc6c6ccccc65)c4)cc3)cc21 ; c1ccc(-c2ccc(N(c3ccc(-c4cccc(-n5c6ccccc6c6ccccc65)c4)cc3)c3ccc4c(c3)oc3ccccc34)cc2)cc1 ; c1ccc(-c2ccc(N(c3ccccc3)c3ccc(-c4cccc(-n5c6ccccc6c6ccccc65)c4)cc3)cc2)cc1]

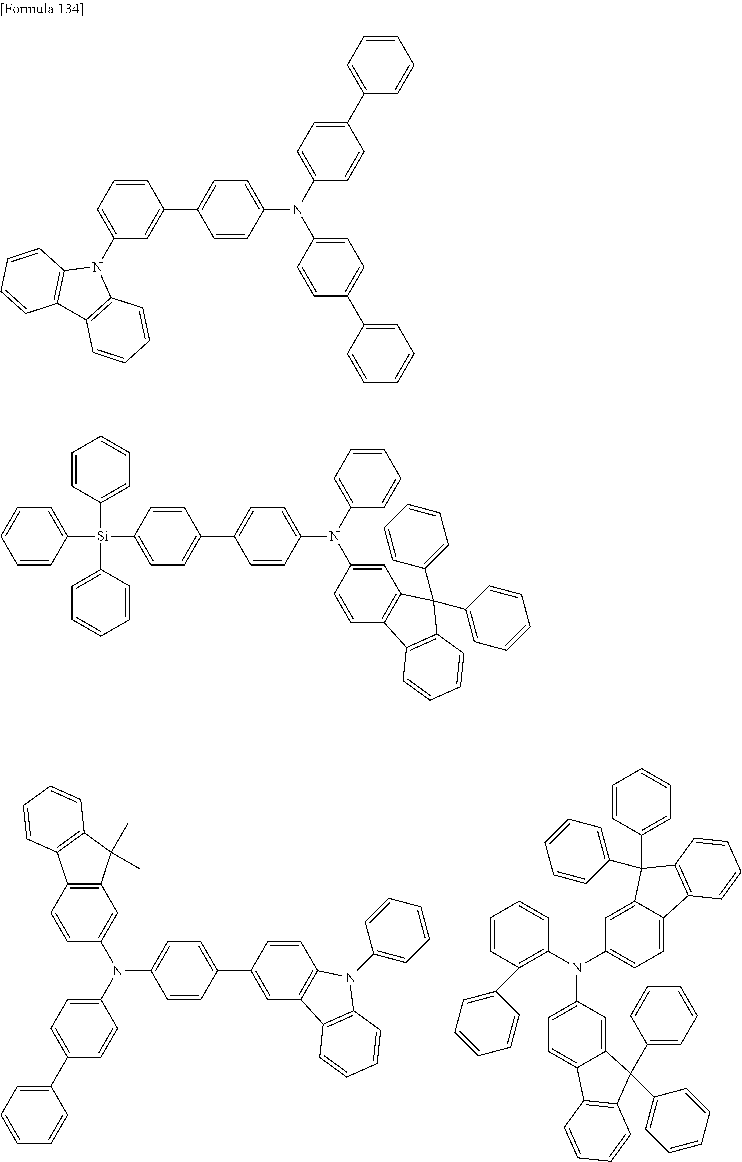 [SMILES: c1ccc(-c2ccc(N(c3ccc(-c4ccccc4)cc3)c3ccc(-c4cccc(-n5c6ccccc6c6ccccc65)c4)cc3)cc2)cc1 ; c1ccc(N(c2ccc(-c3ccc([Si](c4ccccc4)(c4ccccc4)c4ccccc4)cc3)cc2)c2ccc3c(c2)C(c2ccccc2)(c2ccccc2)c2ccccc2-3)cc1 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccccc2N(c2ccc3c(c2)C(c2ccccc2)(c2ccccc2)c2ccccc2-3)c2ccc3c(c2)C(c2ccccc2)(c2ccccc2)c2ccccc2-3)cc1]

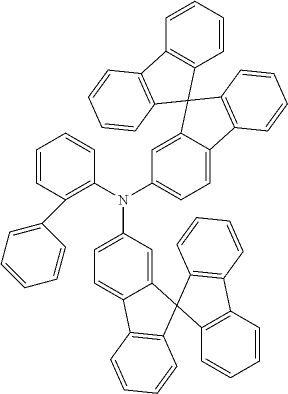 [SMILES: c1ccc(-c2ccccc2N(c2ccc3c(c2)C2(c4ccccc4-c4ccccc42)c2ccccc2-3)c2ccc3c(c2)C2(c4ccccc4-c4ccccc42)c2ccccc2-3)cc1]

In an exemplary arrangement of the organic EL device of the exemplary embodiment, the second organic material contained in the first anode side organic layer (the first hole transporting zone material) is preferably at least one compound selected from compounds below.

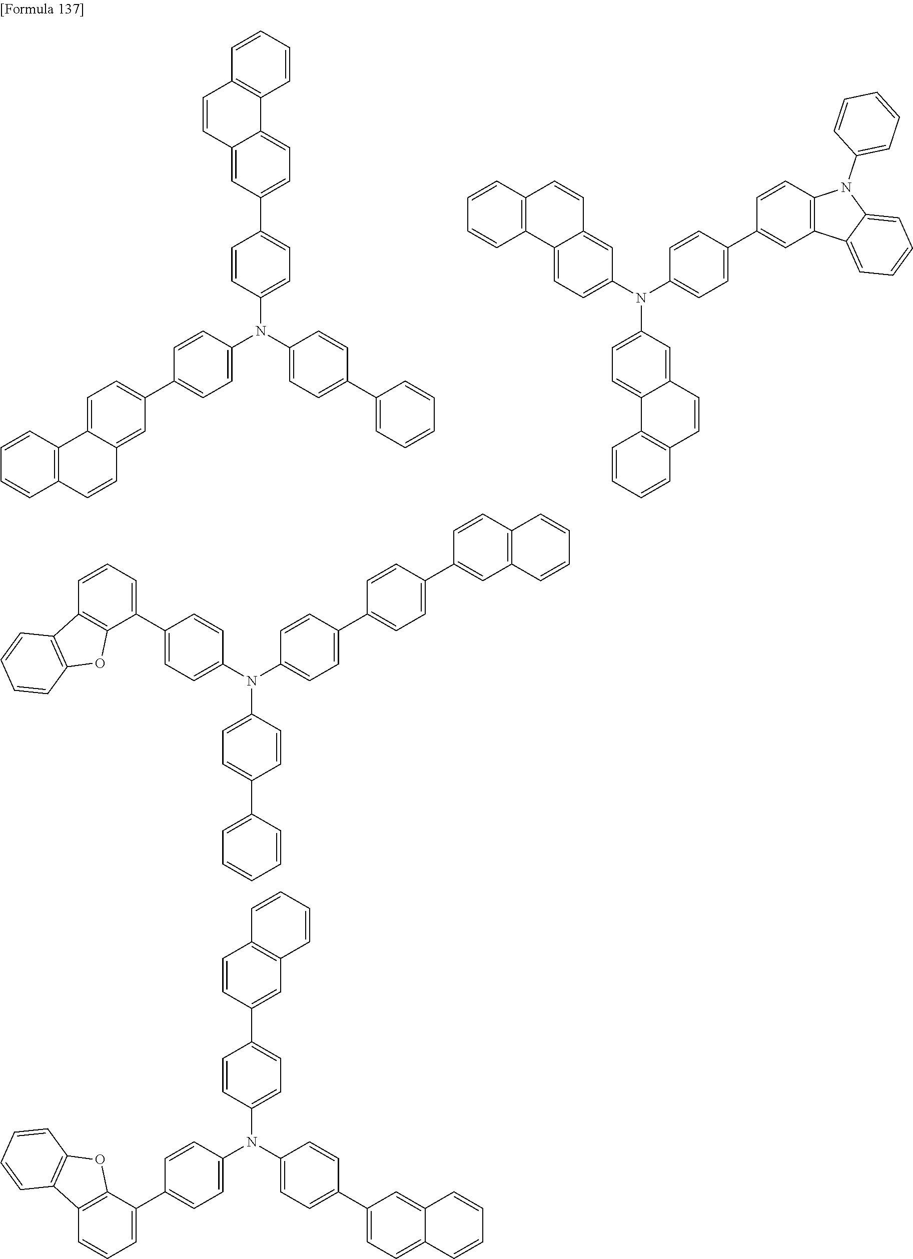

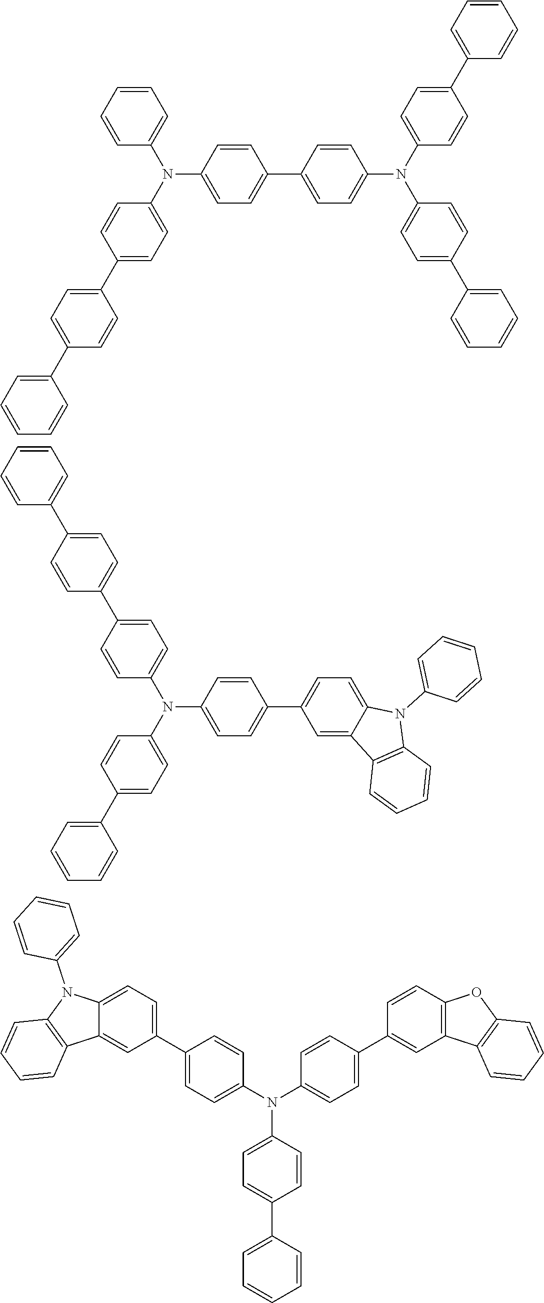

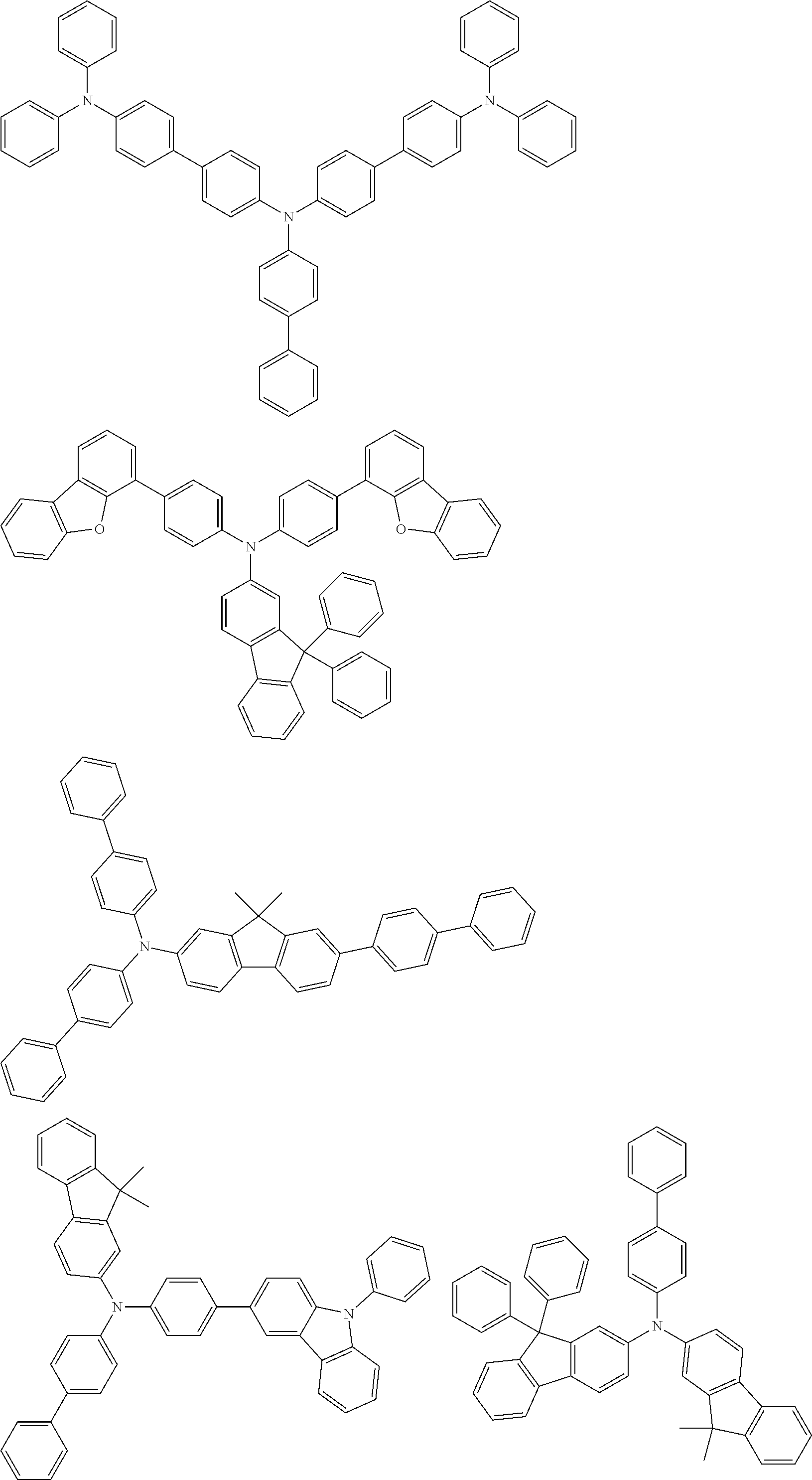

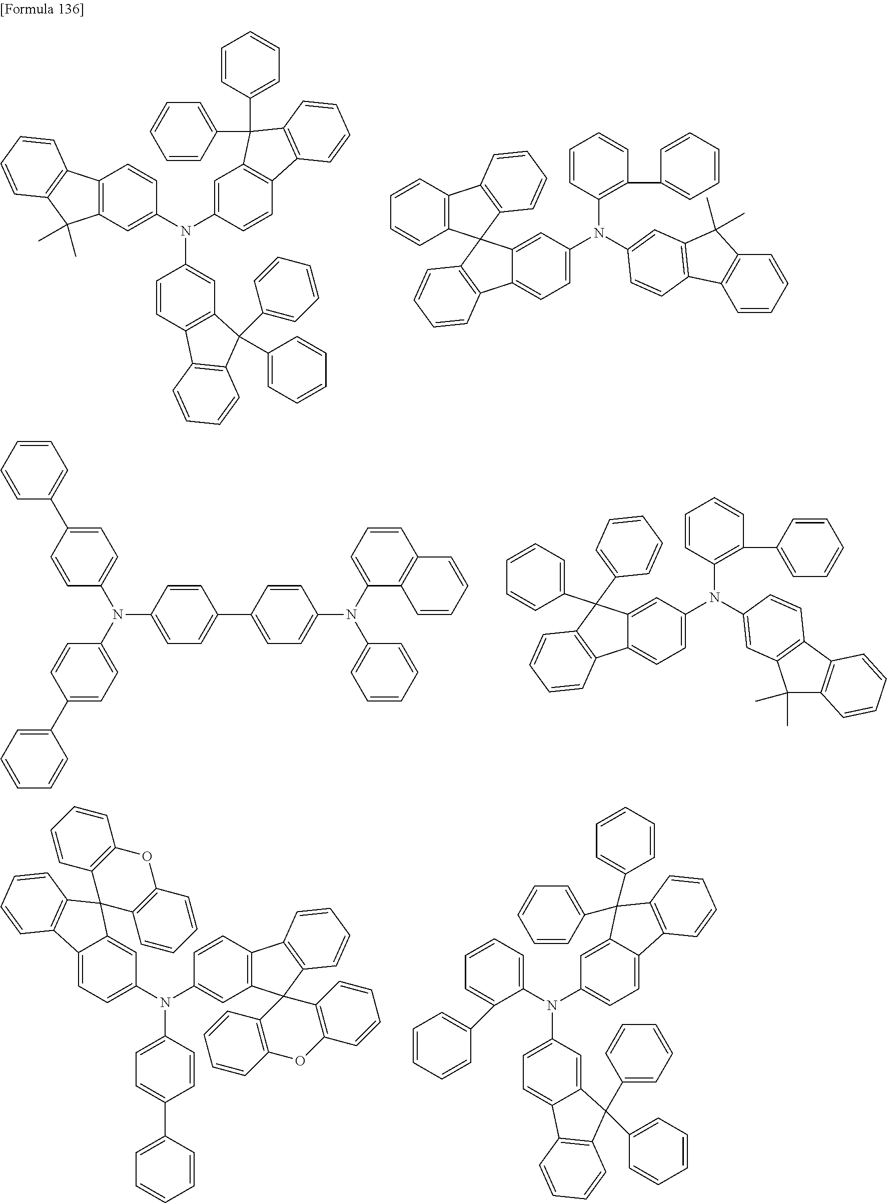

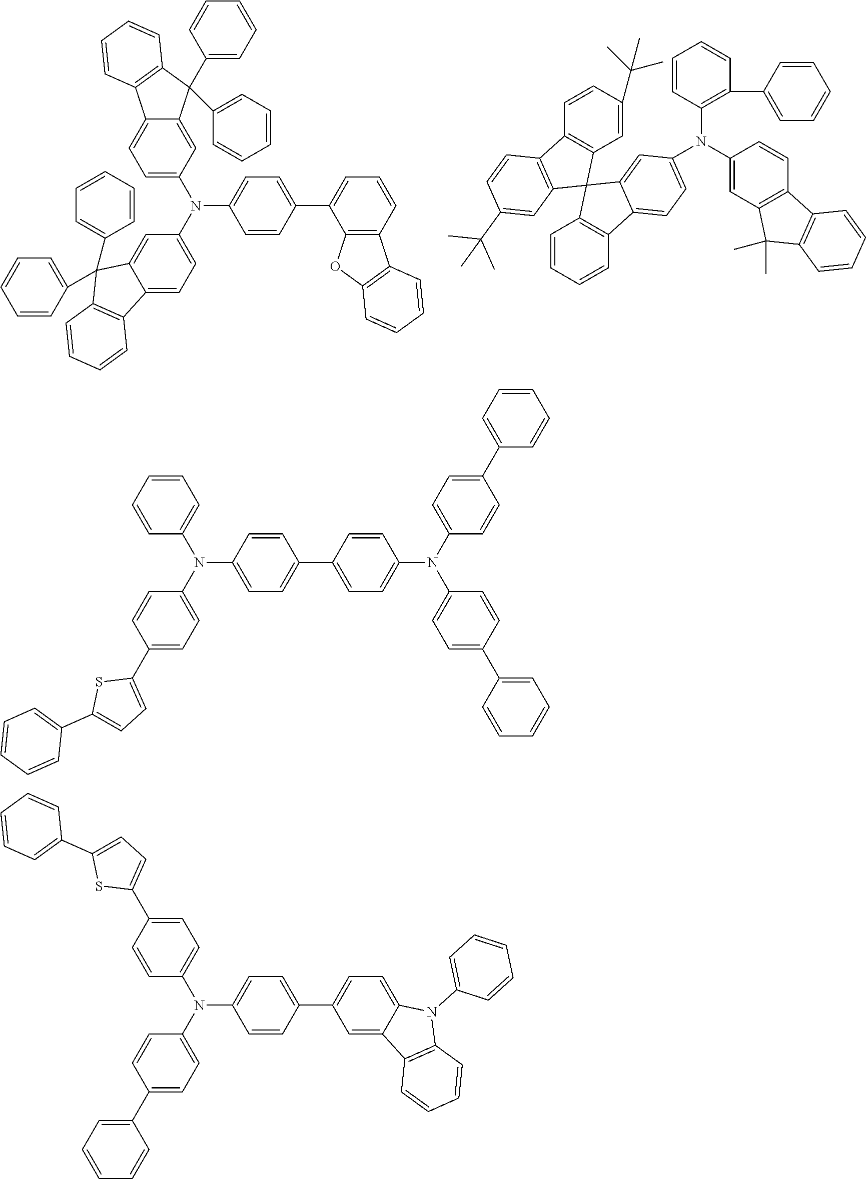

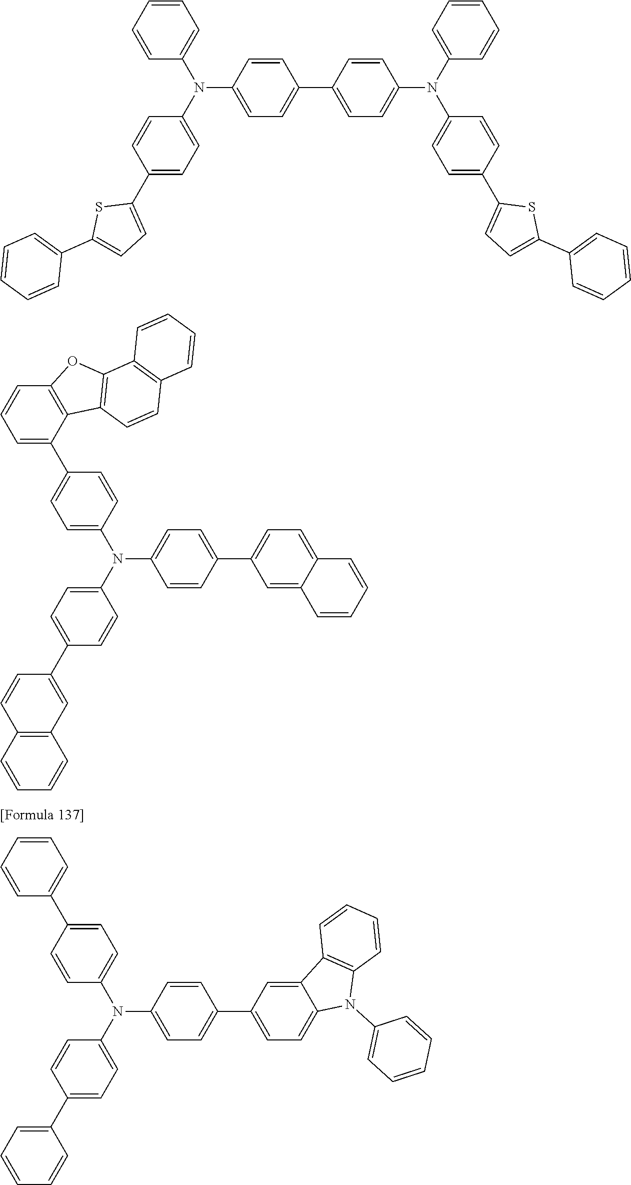 [SMILES: c1ccc(-c2ccc(-c3ccc(N(c4ccccc4)c4ccc(-c5ccc(N(c6ccccc6)c6ccc(-c7ccc(-c8ccccc8)s7)cc6)cc5)cc4)cc3)s2)cc1 ; c1ccc2cc(-c3ccc(N(c4ccc(-c5ccc6ccccc6c5)cc4)c4ccc(-c5cccc6oc7c8ccccc8ccc7c56)cc4)cc3)ccc2c1 ; c1ccc(-c2ccc(N(c3ccc(-c4ccccc4)cc3)c3ccc(-c4ccc5c(c4)c4ccccc4n5-c4ccccc4)cc3)cc2)cc1]

In an exemplary arrangement of the organic EL device of the exemplary embodiment, the compound contained in the second anode side organic layer (the second hole transporting zone material) is preferably at least one compound selected from compounds below.

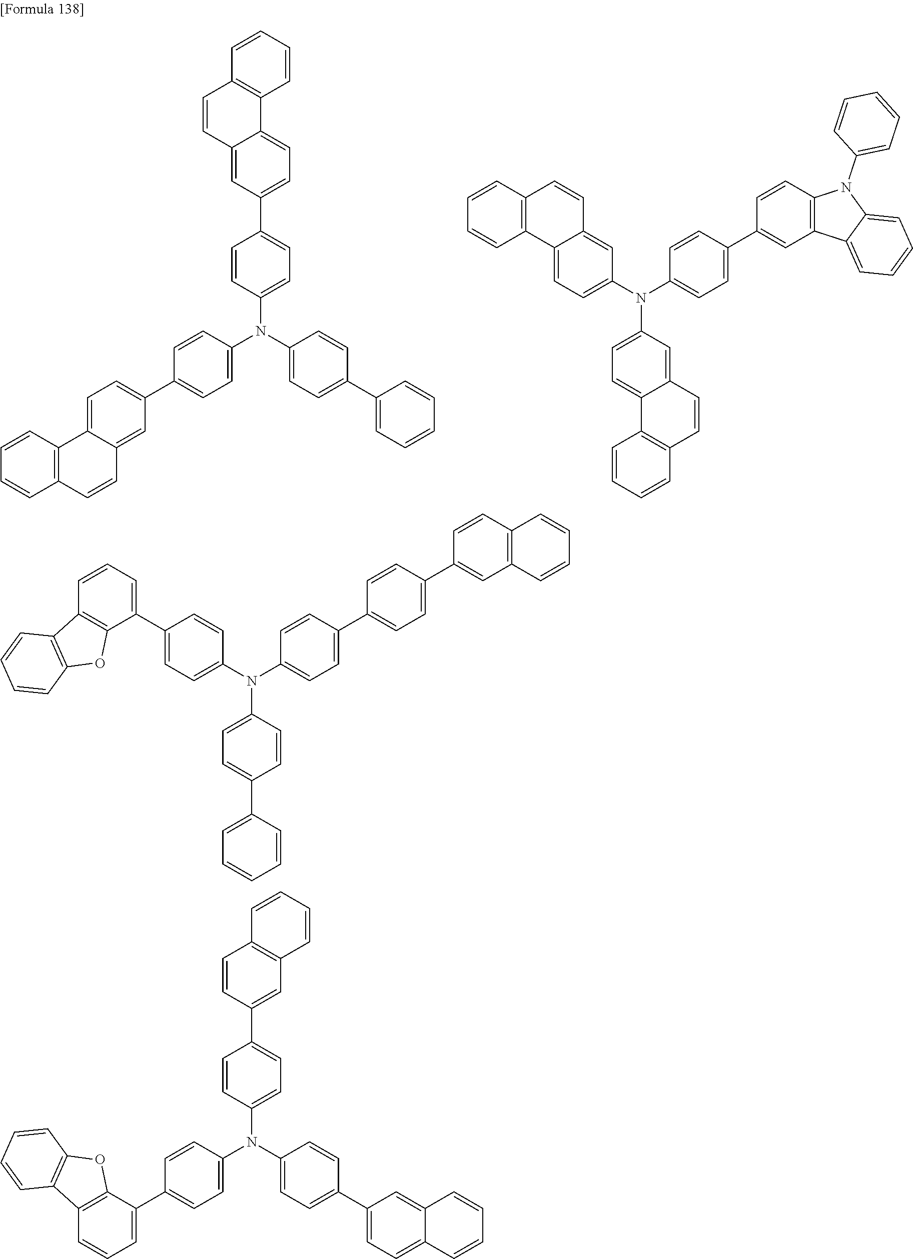

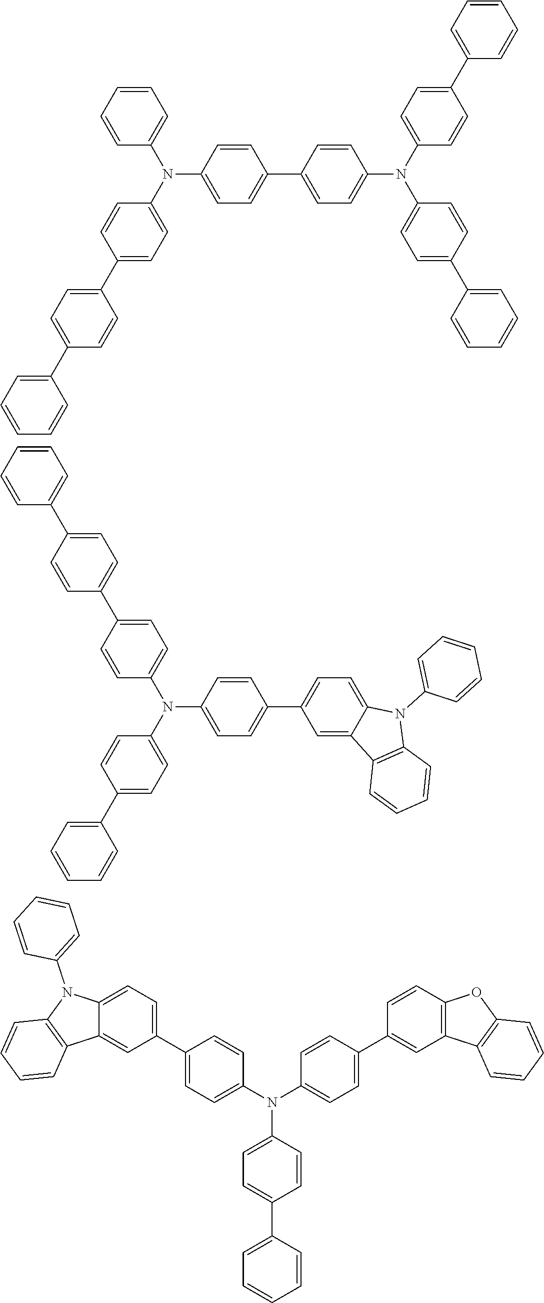

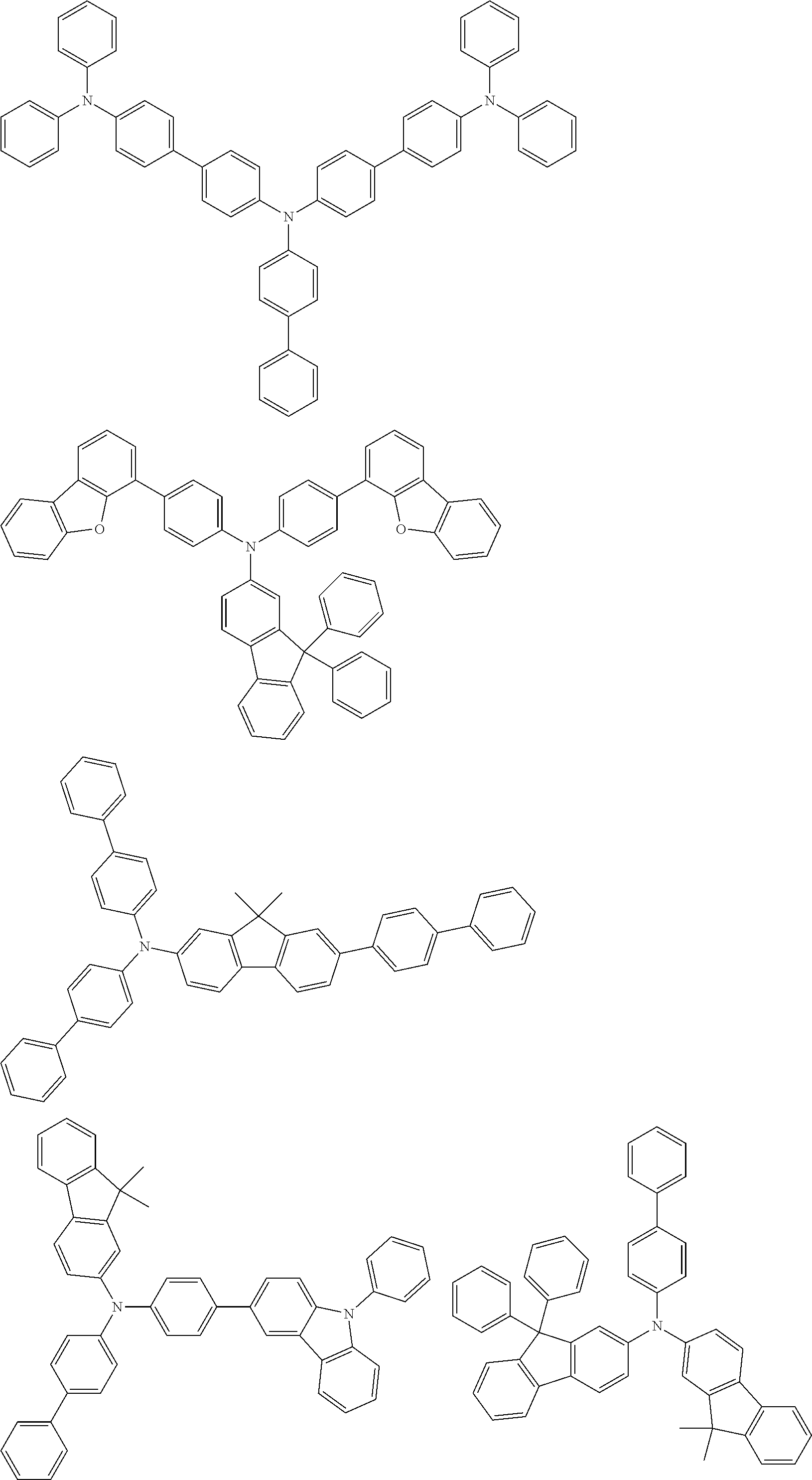

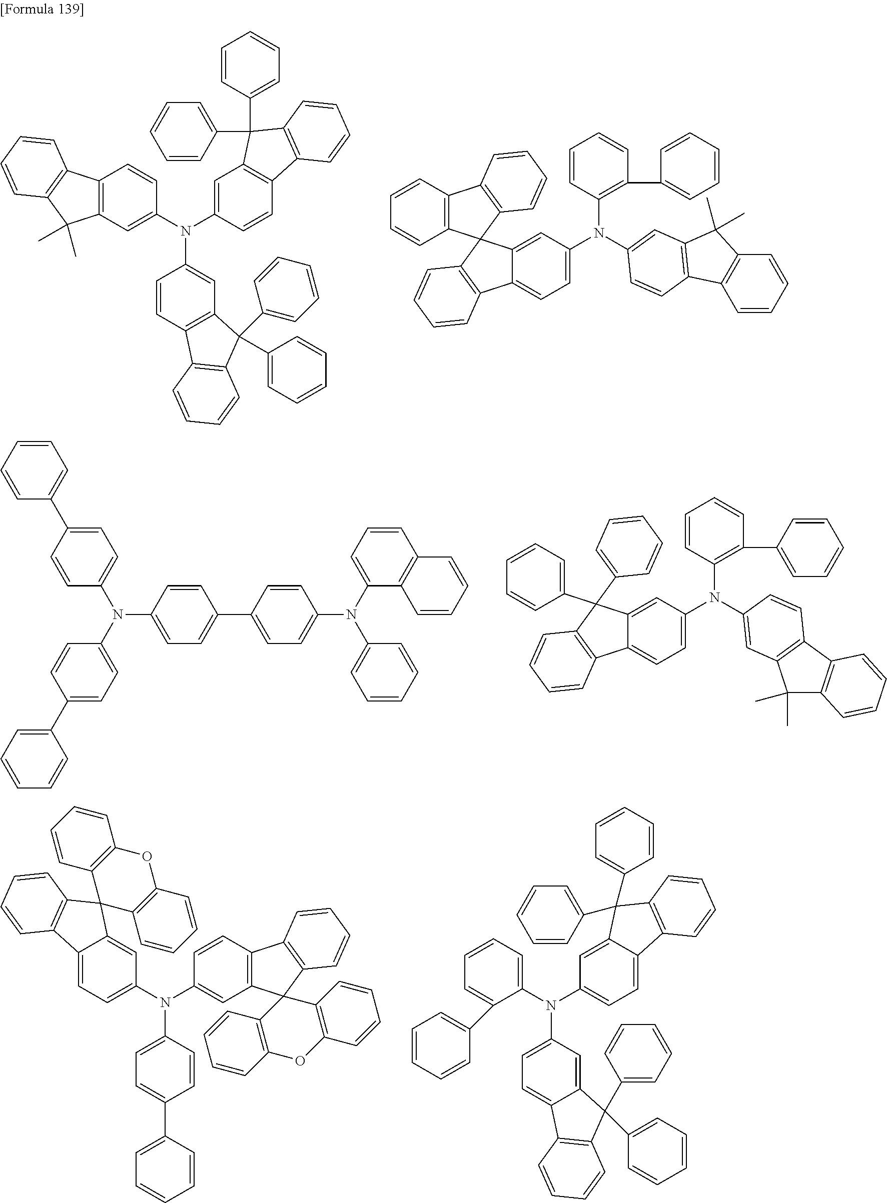

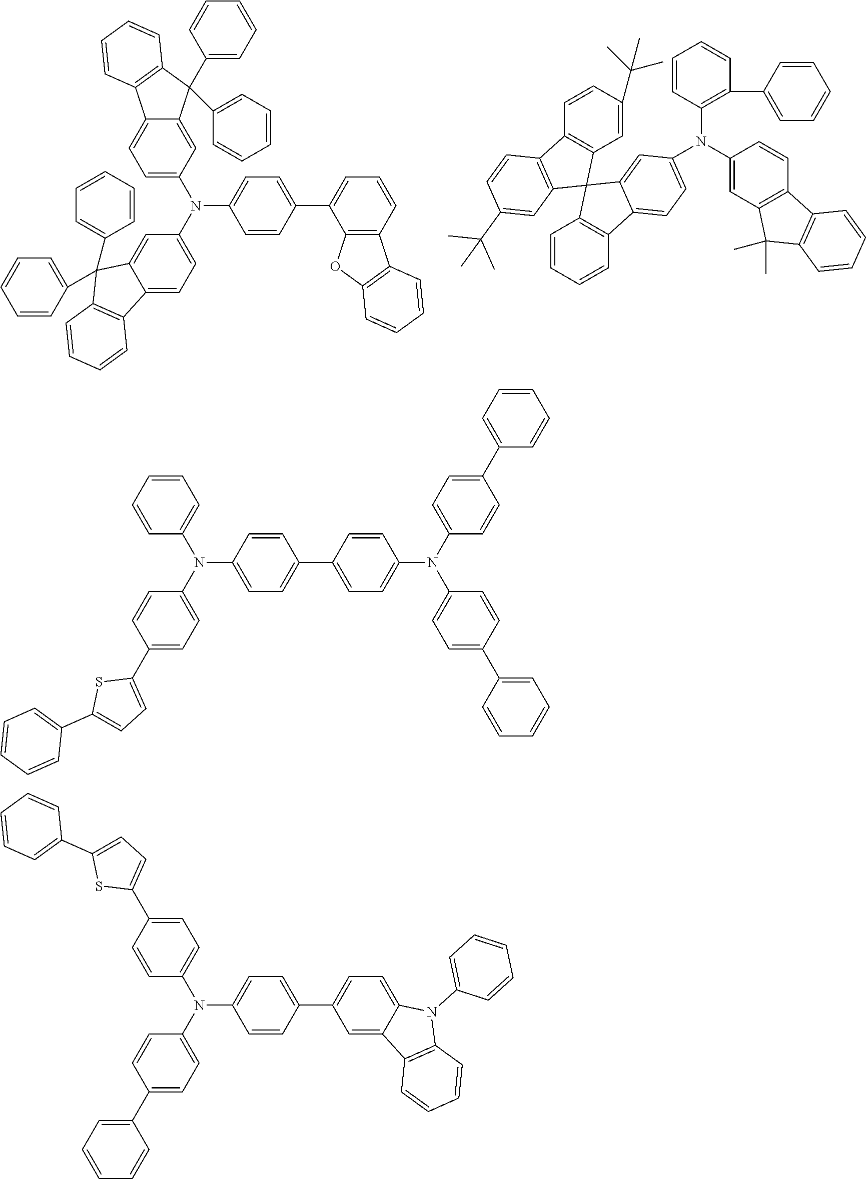

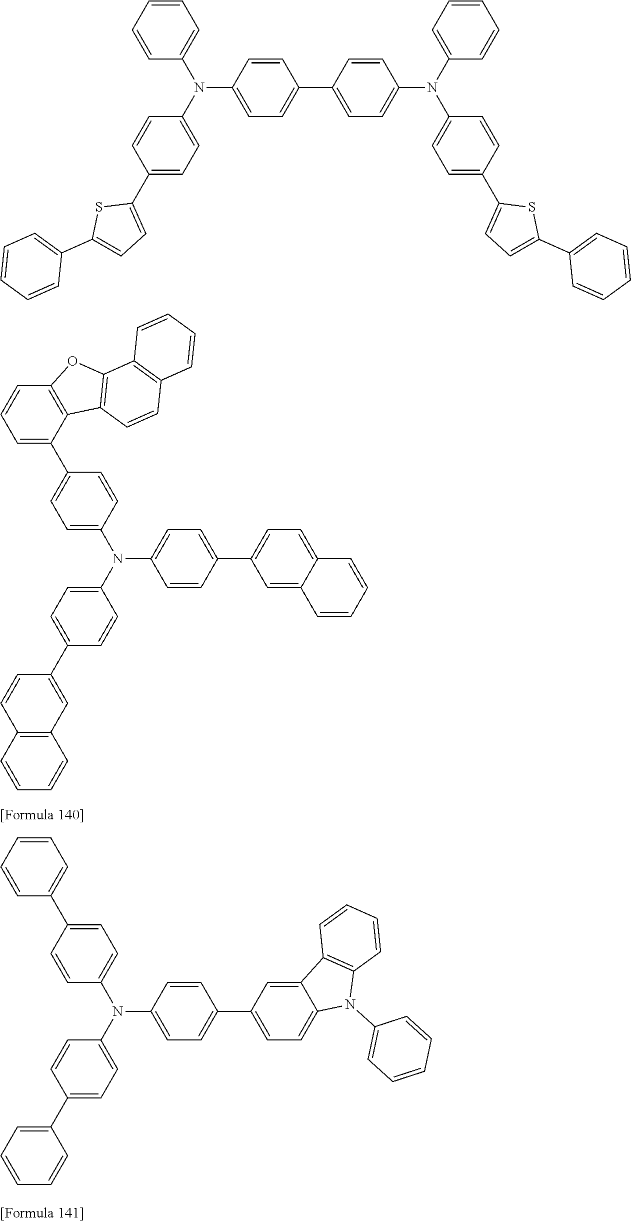 [SMILES: c1ccc(-c2ccc(-c3ccc(N(c4ccccc4)c4ccc(-c5ccc(N(c6ccccc6)c6ccc(-c7ccc(-c8ccccc8)s7)cc6)cc5)cc4)cc3)s2)cc1 ; c1ccc2cc(-c3ccc(N(c4ccc(-c5ccc6ccccc6c5)cc4)c4ccc(-c5cccc6oc7c8ccccc8ccc7c56)cc4)cc3)ccc2c1 ; c1ccc(-c2ccc(N(c3ccc(-c4ccccc4)cc3)c3ccc(-c4ccc5c(c4)c4ccccc4n5-c4ccccc4)cc3)cc2)cc1]

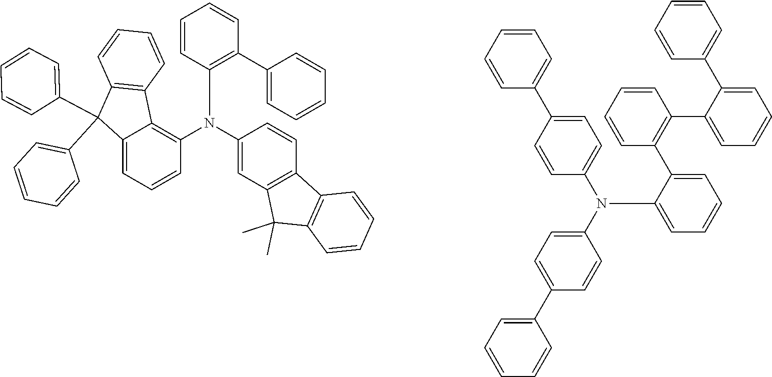 [SMILES: CC1(C)c2ccccc2-c2ccc(N(c3ccccc3-c3ccccc3)c3cccc4c3-c3ccccc3C4(c3ccccc3)c3ccccc3)cc21 ; c1ccc(-c2ccc(N(c3ccc(-c4ccccc4)cc3)c3ccccc3-c3ccccc3-c3ccccc3-c3ccccc3)cc2)cc1]

In an exemplary arrangement of the organic EL device of the exemplary embodiment, the compound contained in the third anode side organic layer (the third hole transporting zone material) is preferably at least one compound selected from compounds below.

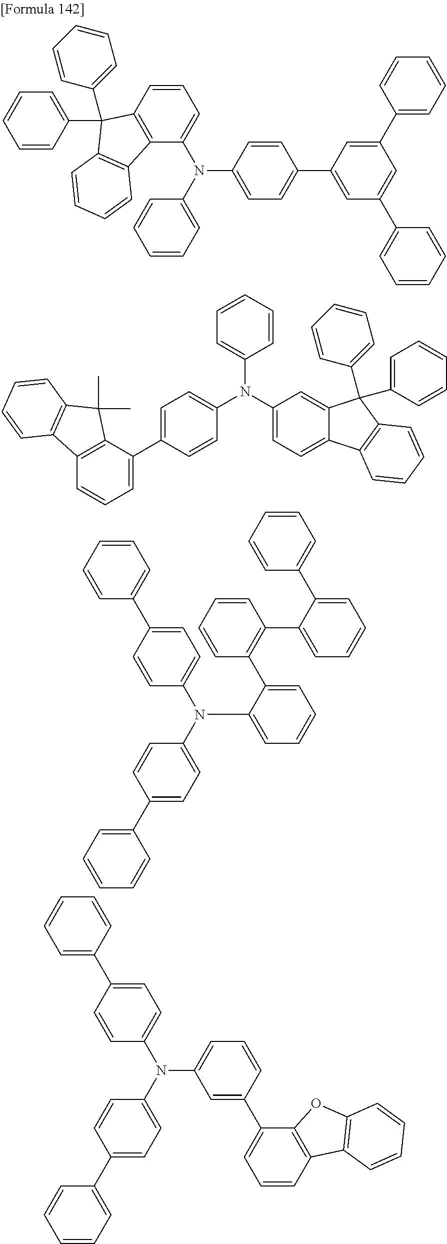

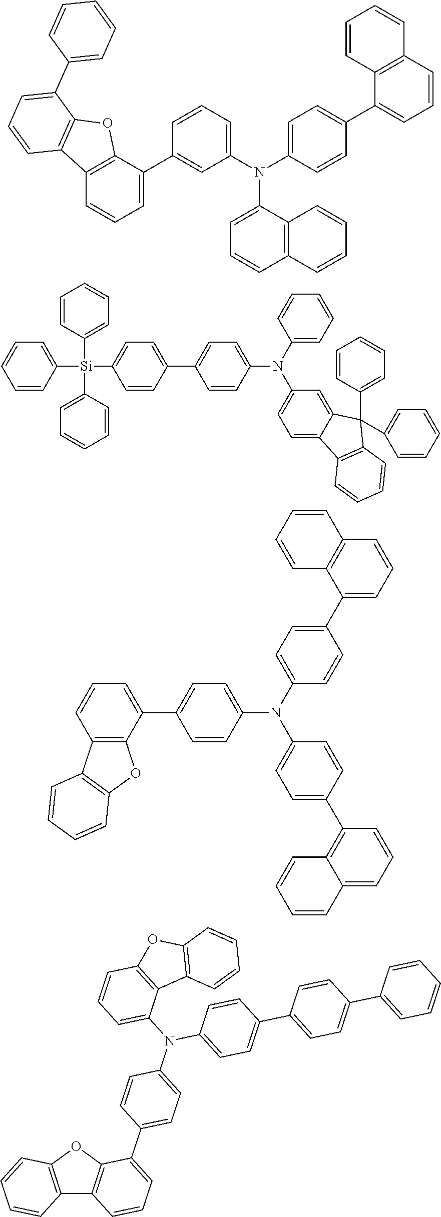

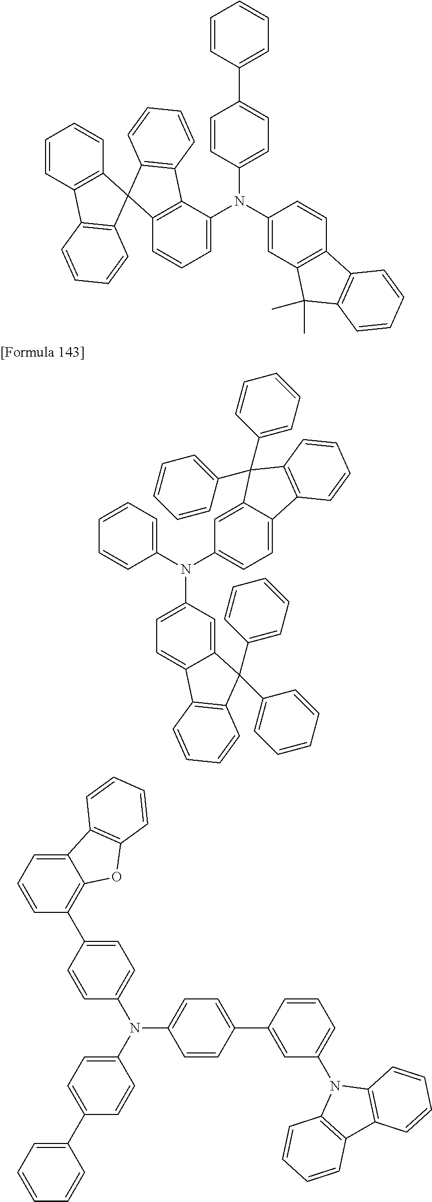 [SMILES: CC1(C)c2ccccc2-c2ccc(N(c3ccc(-c4ccccc4)cc3)c3cccc4c3-c3ccccc3C43c4ccccc4-c4ccccc43)cc21 ; c1ccc(N(c2ccc3c(c2)C(c2ccccc2)(c2ccccc2)c2ccccc2-3)c2ccc3c(c2)C(c2ccccc2)(c2ccccc2)c2ccccc2-3)cc1 ; c1ccc(-c2ccc(N(c3ccc(-c4cccc(-n5c6ccccc6c6ccccc65)c4)cc3)c3ccc(-c4cccc5c4oc4ccccc45)cc3)cc2)cc1]

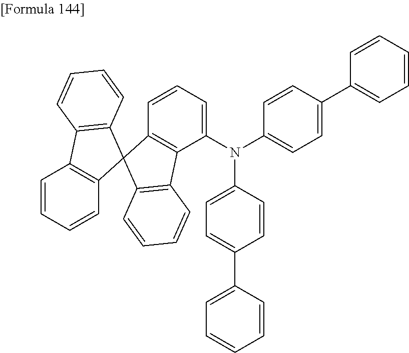 [SMILES: c1ccc(-c2ccc(N(c3ccc(-c4ccccc4)cc3)c3cccc4c3-c3ccccc3C43c4ccccc4-c4ccccc43)cc2)cc1]

It should be noted that exemplary compounds described as compounds contained in the first anode side organic layer, the second anode side organic layer, and the third anode side organic layer may overlap with each other; however, in the exemplary embodiment, different compounds can be appropriately selected from among those exemplary compounds, as compounds usable in the first anode side organic layer, the second anode side organic layer and the third anode side organic layer.

### Specific Examples of Doped Compound

Specific examples of the doped compound as the first organic material include the following compounds. It should however be noted that the invention is not limited to the specific examples of the doped compound.

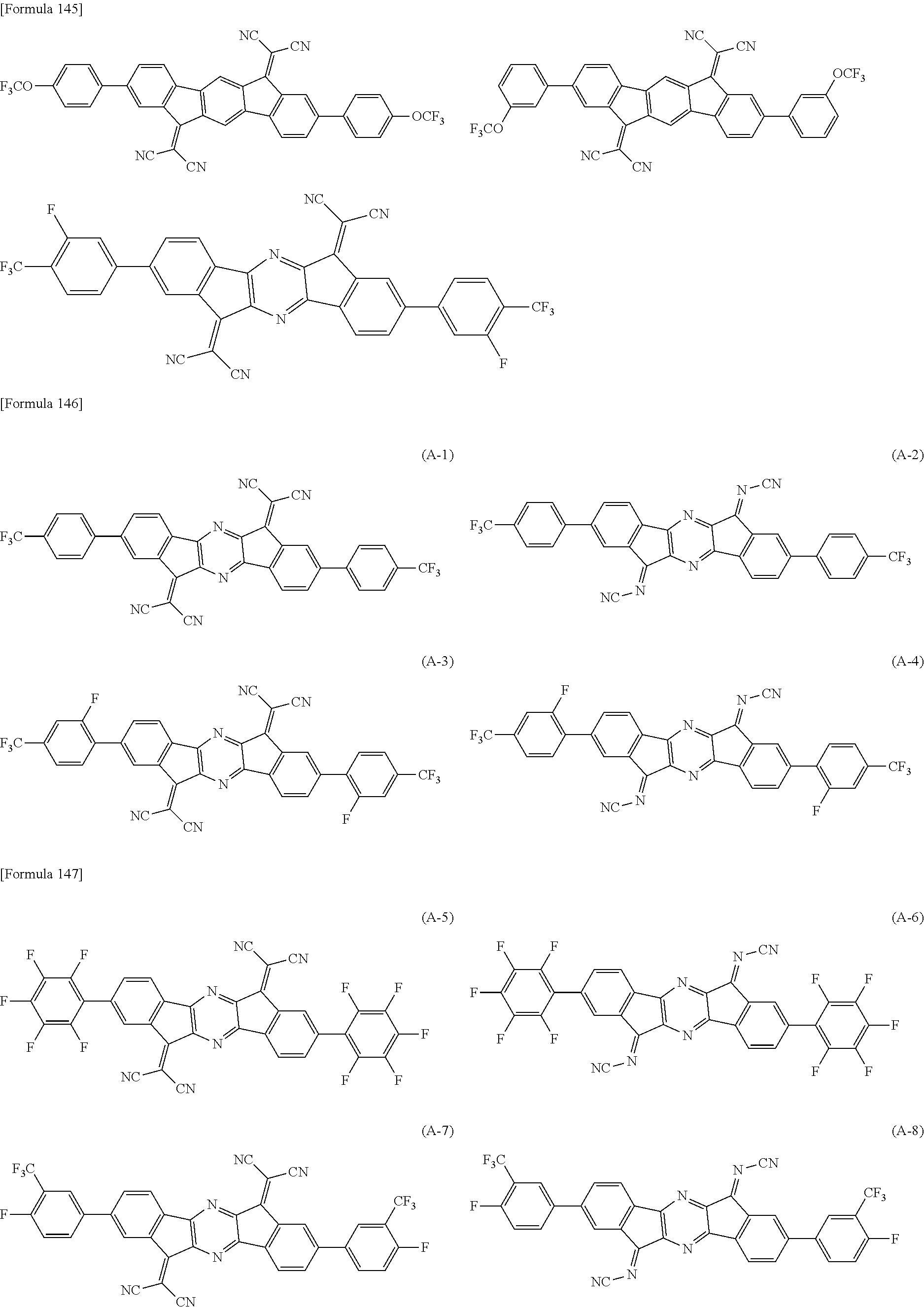

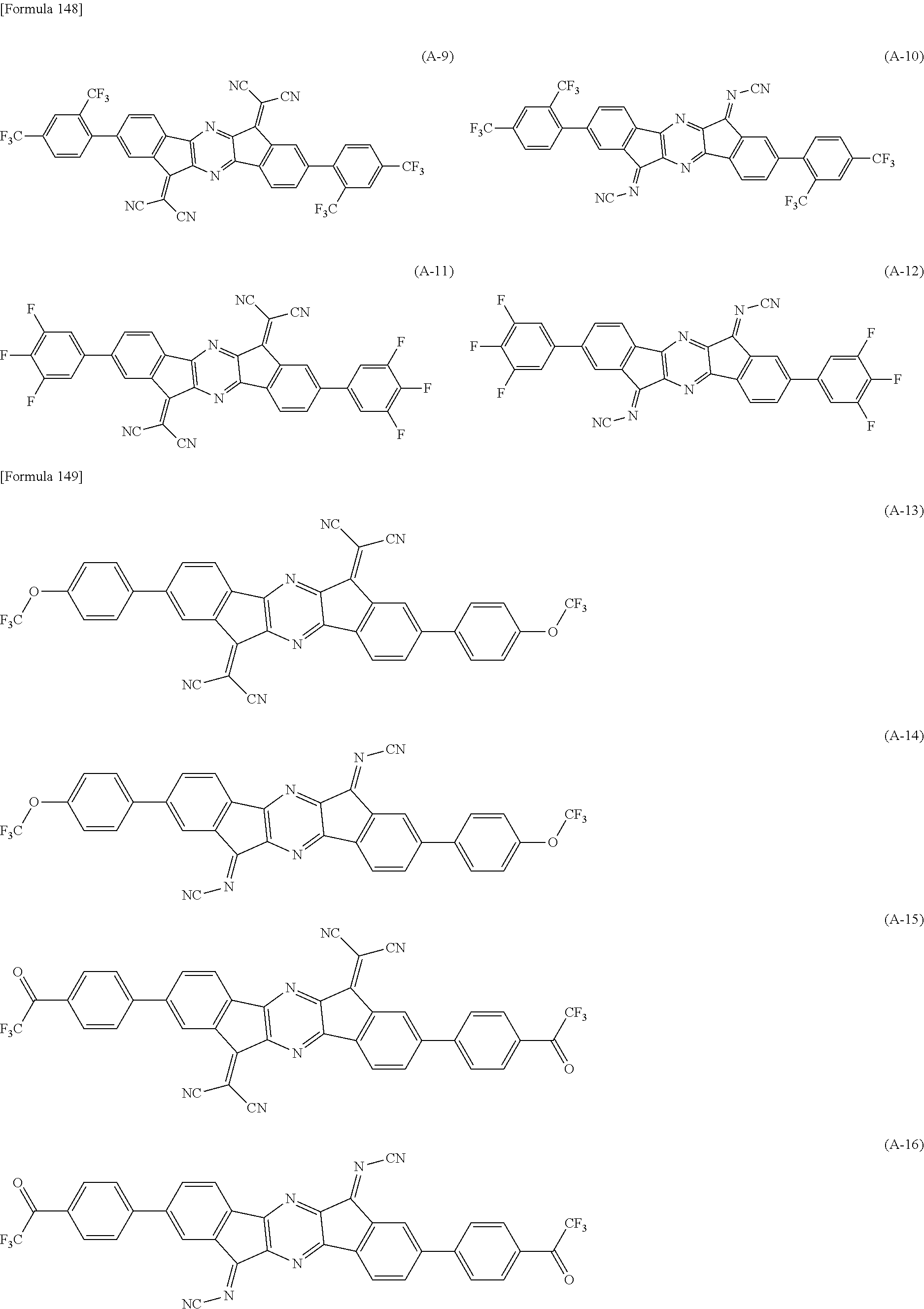

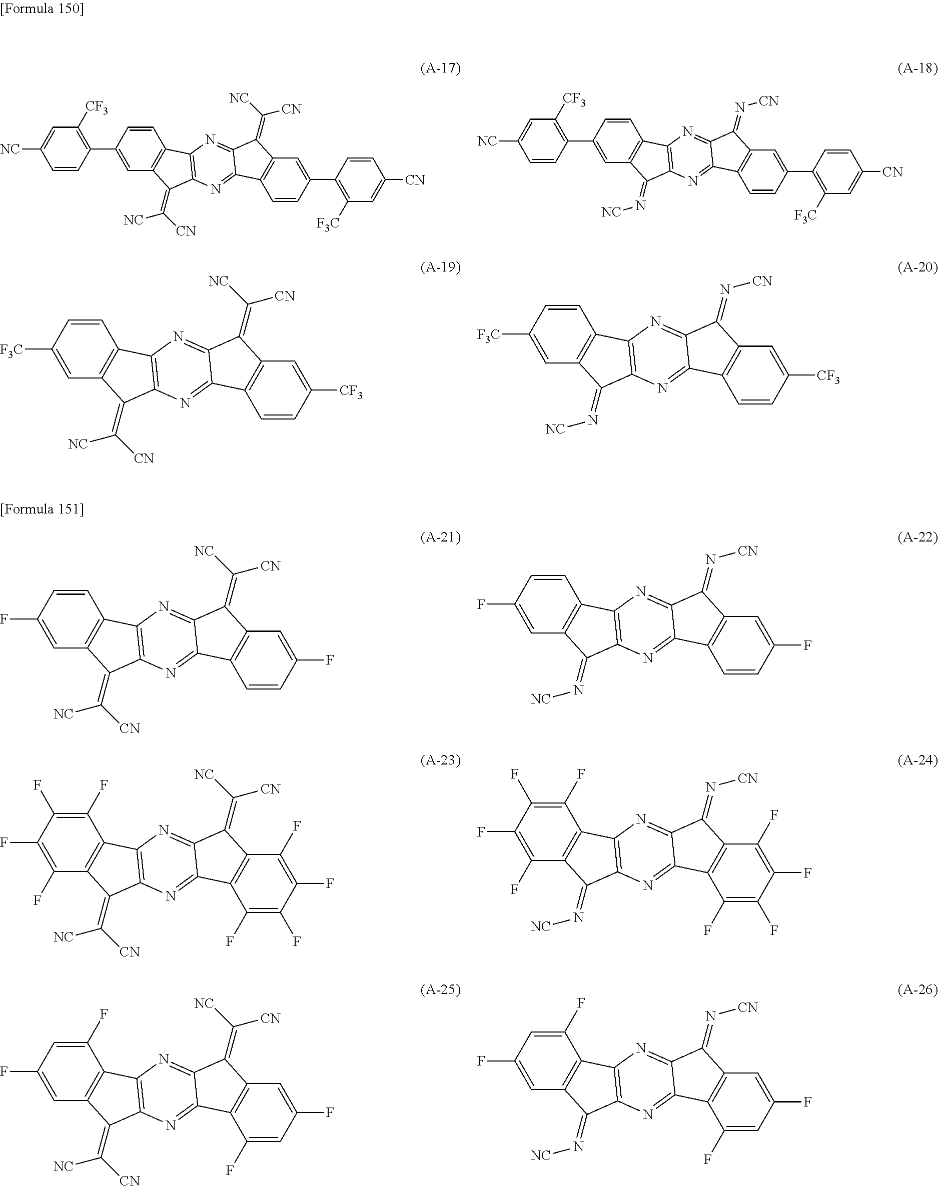

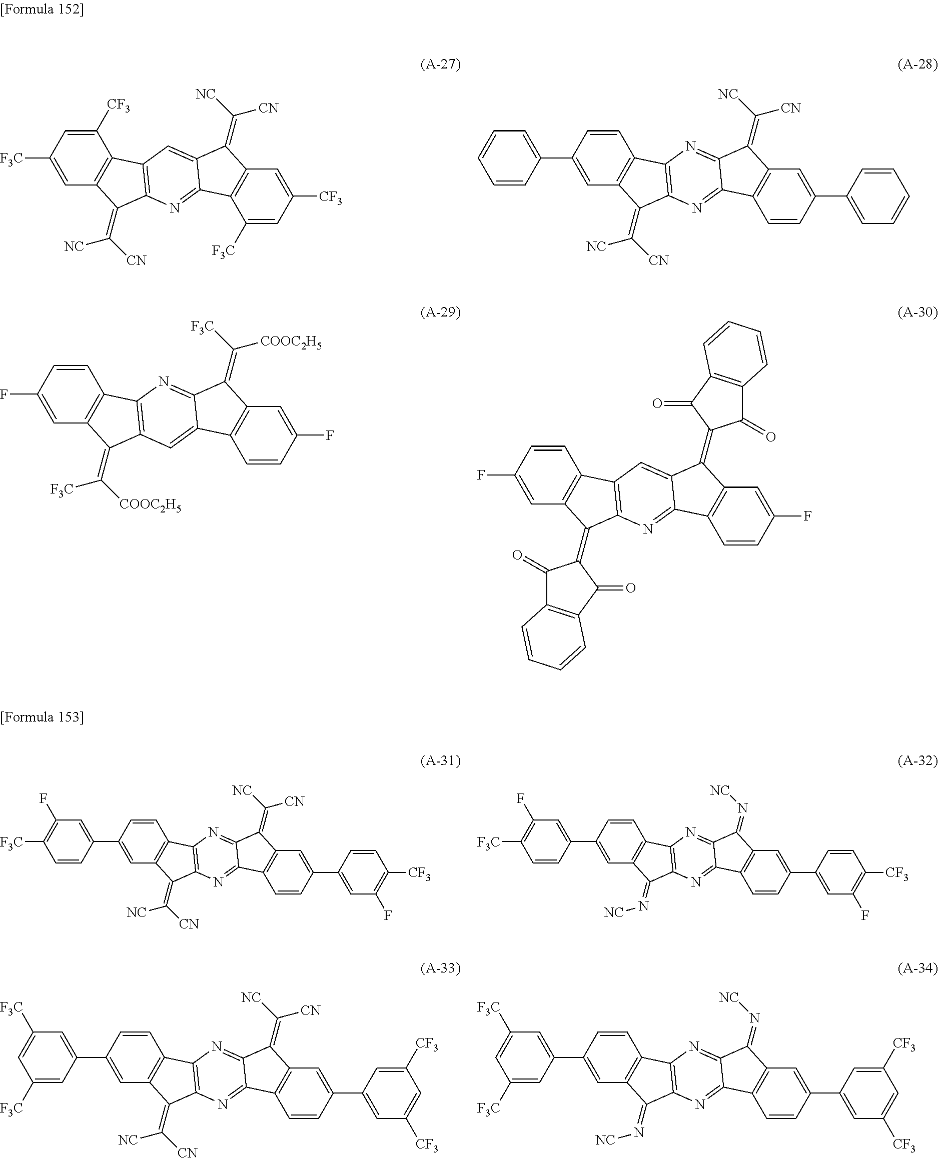

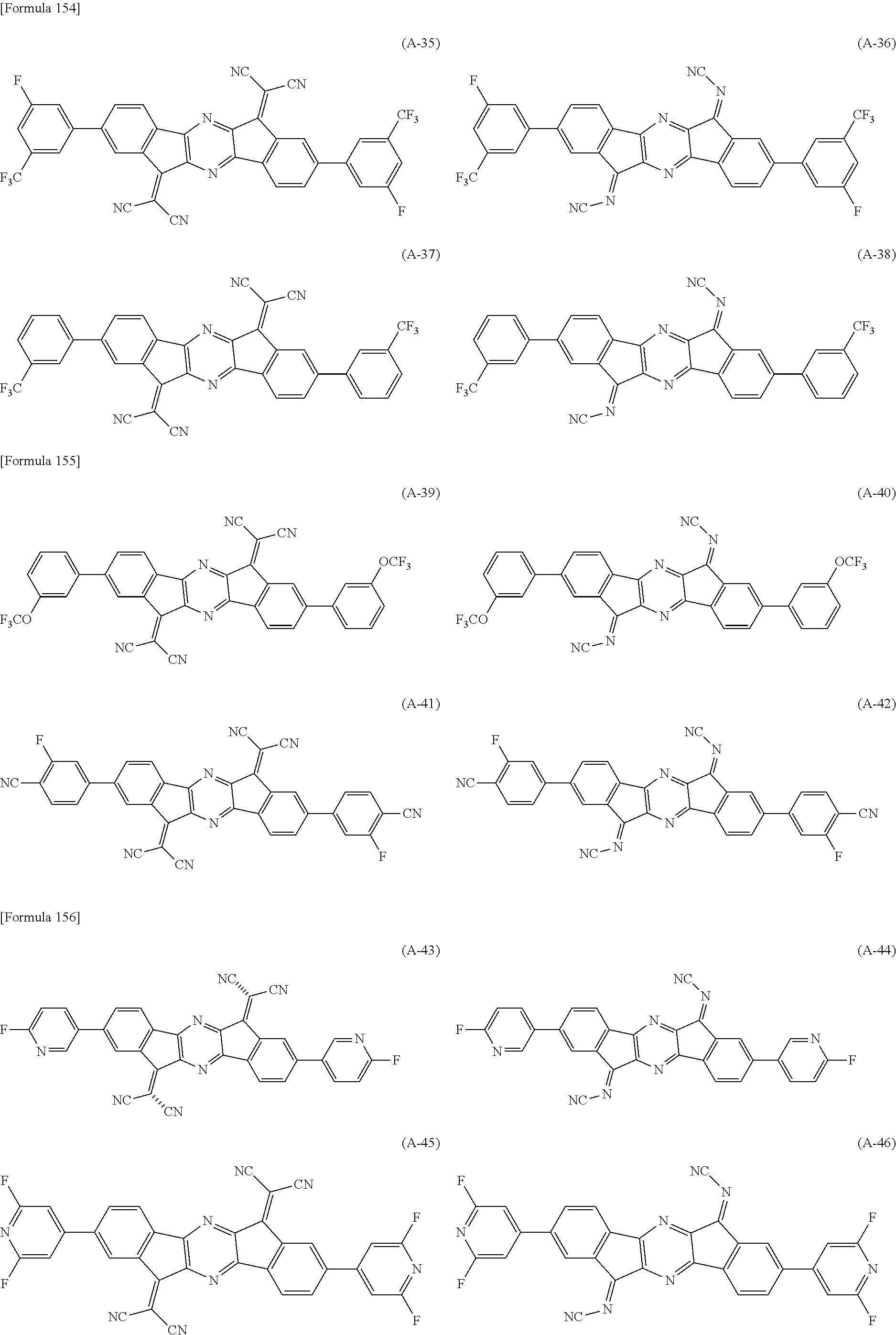

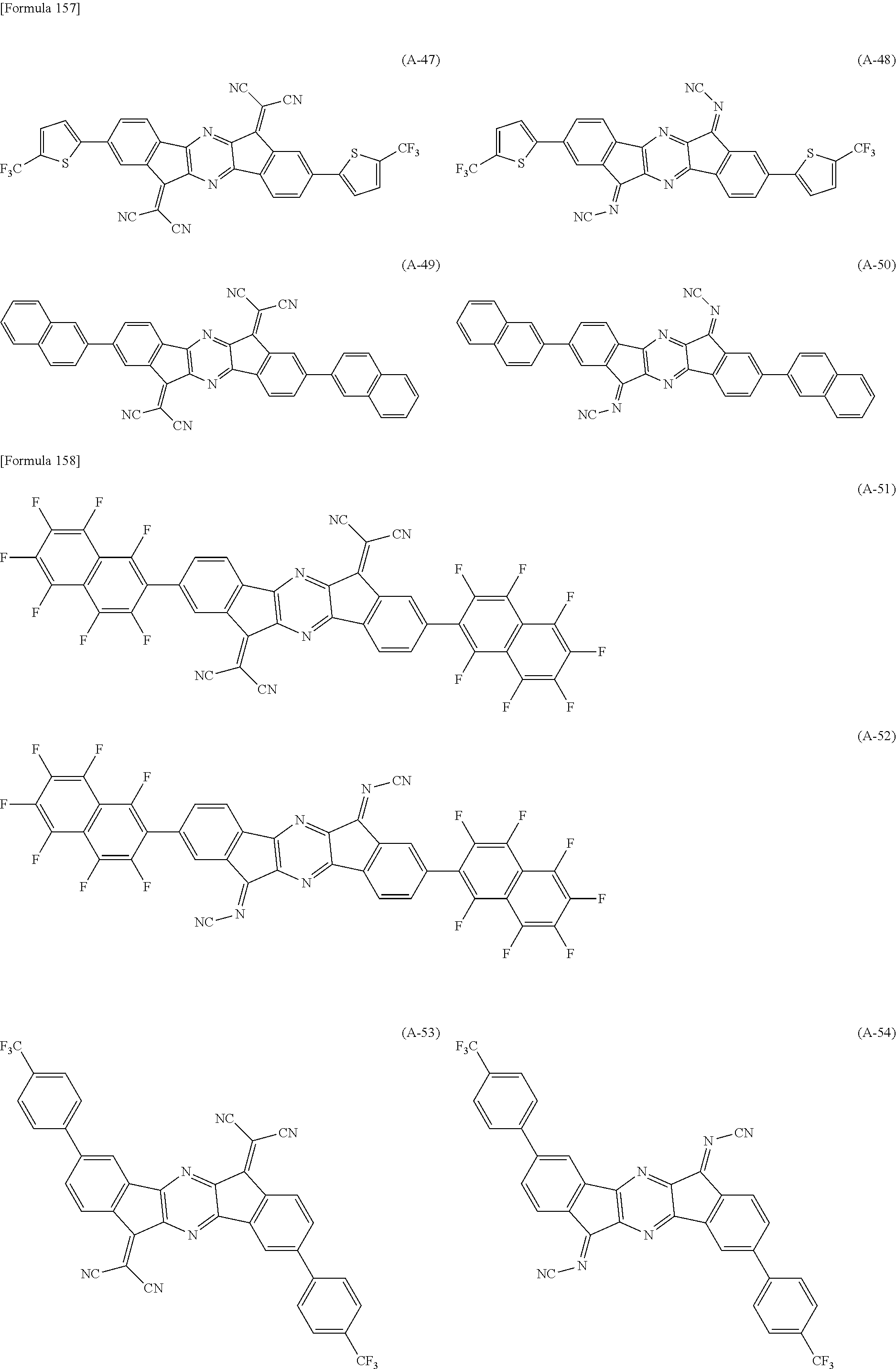

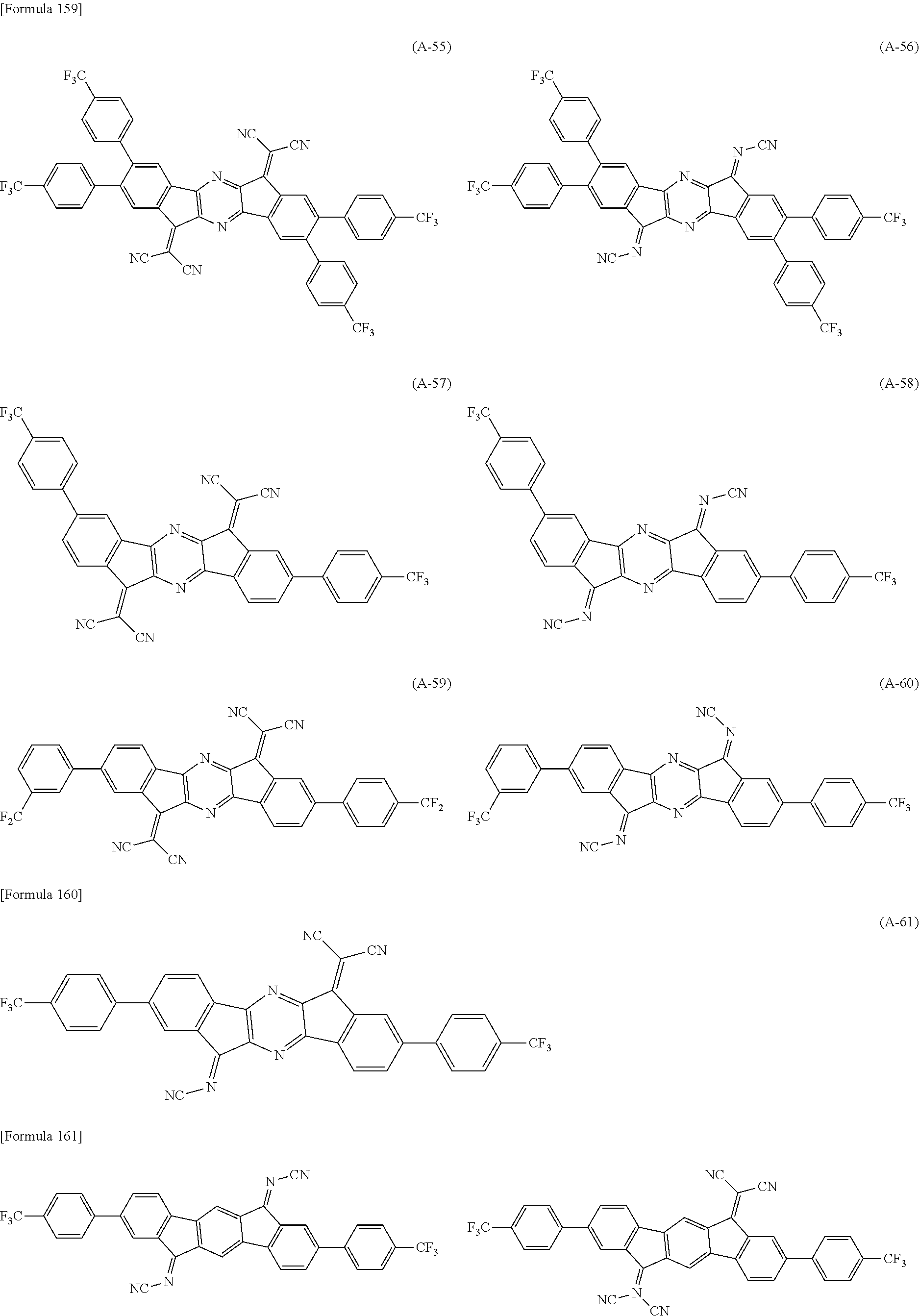

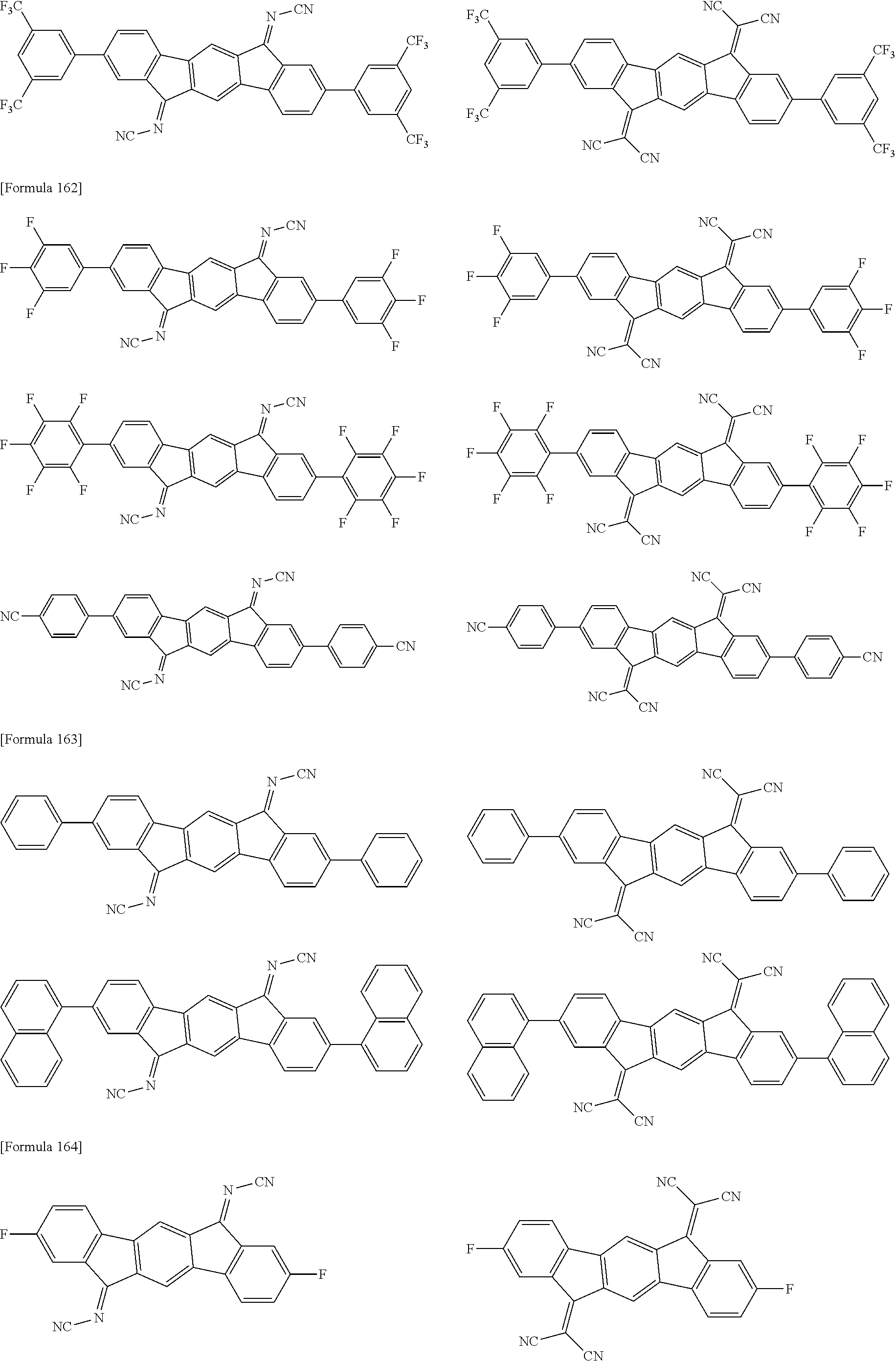

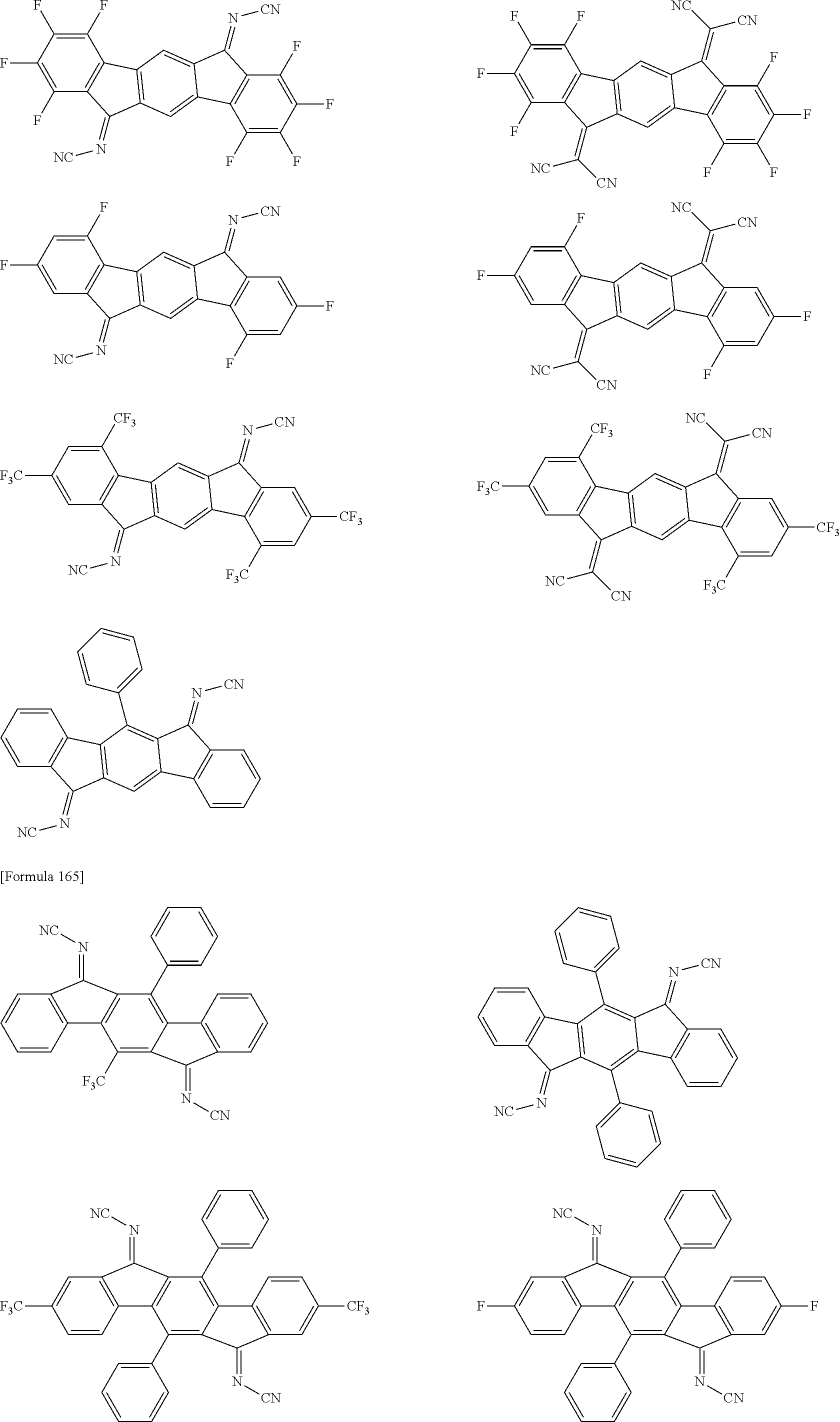

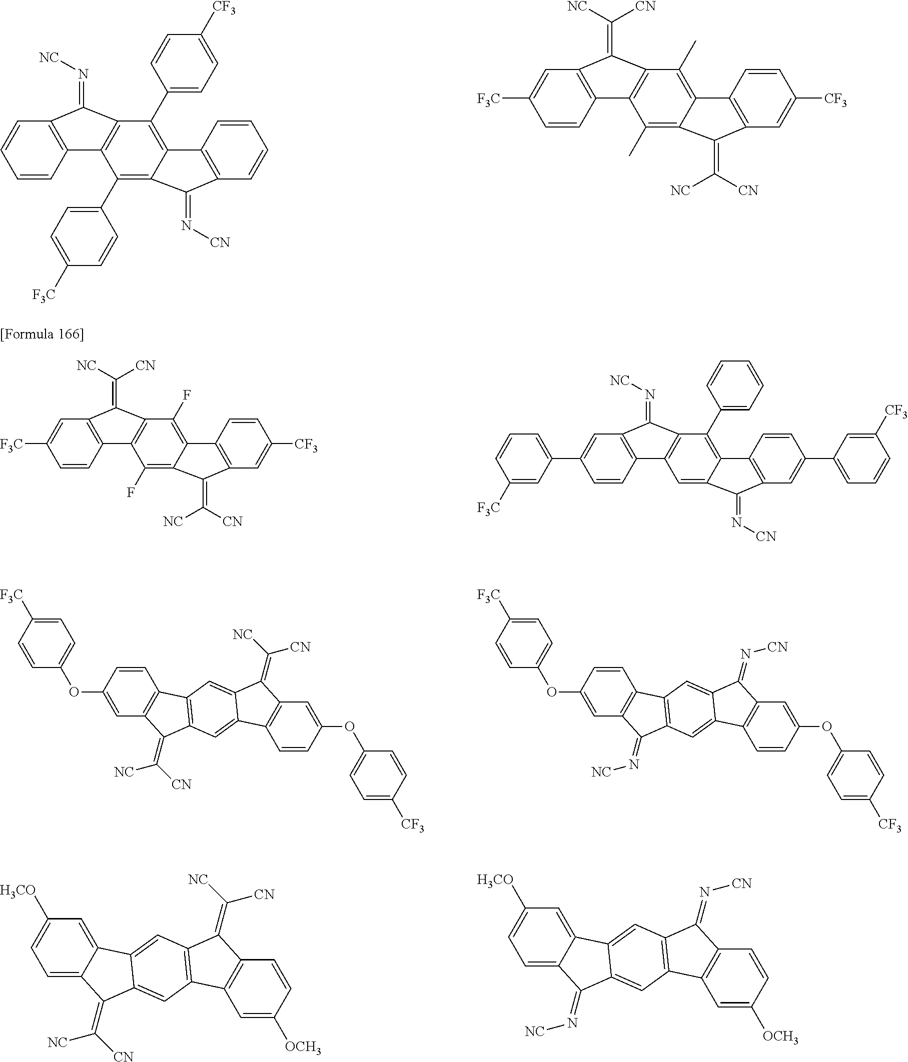

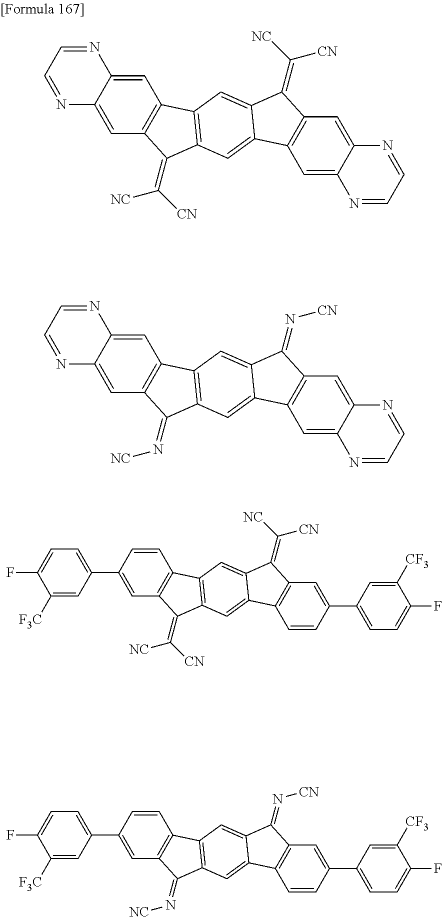 [SMILES: N#CC(C#N)=C1c2cc3c(cc2-c2cc4nccnc4cc21)C(=C(C#N)C#N)c1cc2nccnc2cc1-3 ; N#C/N=c1\c2cc3nccnc3cc2c2cc3/c(=N/C#N)c4cc5nccnc5cc4c3cc12 ; N#CC(C#N)=C1c2cc(-c3ccc(F)c(C(F)(F)F)c3)ccc2-c2cc3c(cc21)-c1ccc(-c2ccc(F)c(C(F)(F)F)c2)cc1C3=C(C#N)C#N ; N#C/N=c1\c2cc(-c3ccc(F)c(C(F)(F)F)c3)ccc2c2cc3/c(=N/C#N)c4cc(-c5ccc(F)c(C(F)(F)F)c5)ccc4c3cc12]

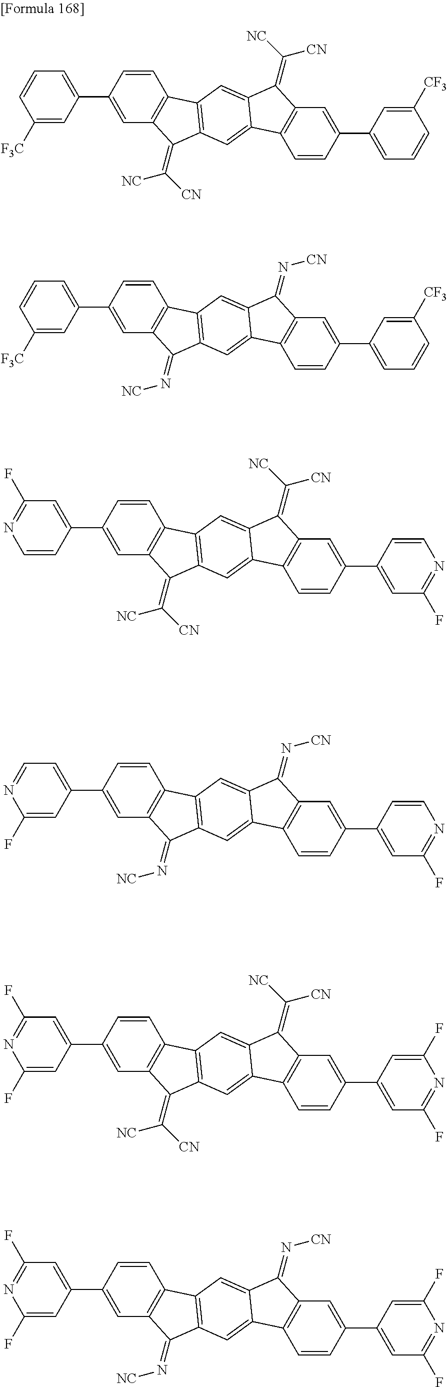

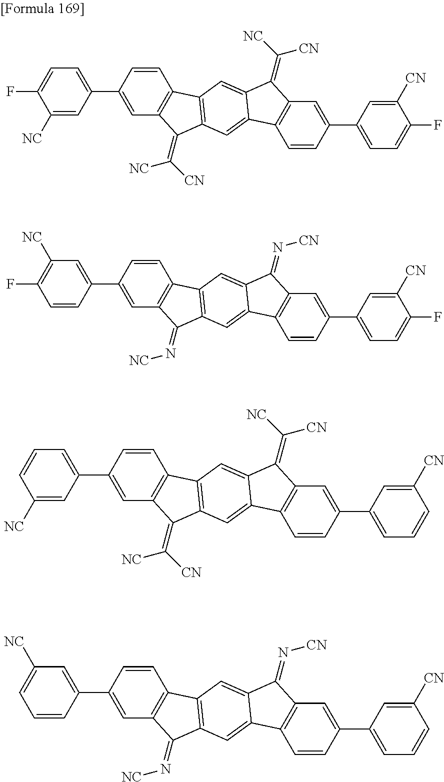 [SMILES: N#CC(C#N)=C1c2cc(-c3ccc(F)c(C#N)c3)ccc2-c2cc3c(cc21)-c1ccc(-c2ccc(F)c(C#N)c2)cc1C3=C(C#N)C#N ; N#C/N=c1\c2cc(-c3ccc(F)c(C#N)c3)ccc2c2cc3/c(=N/C#N)c4cc(-c5ccc(F)c(C#N)c5)ccc4c3cc12 ; N#CC(C#N)=C1c2cc(-c3cccc(C#N)c3)ccc2-c2cc3c(cc21)-c1ccc(-c2cccc(C#N)c2)cc1C3=C(C#N)C#N ; N#C/N=c1\c2cc(-c3cccc(C#N)c3)ccc2c2cc3/c(=N/C#N)c4cc(-c5cccc(C#N)c5)ccc4c3cc12]

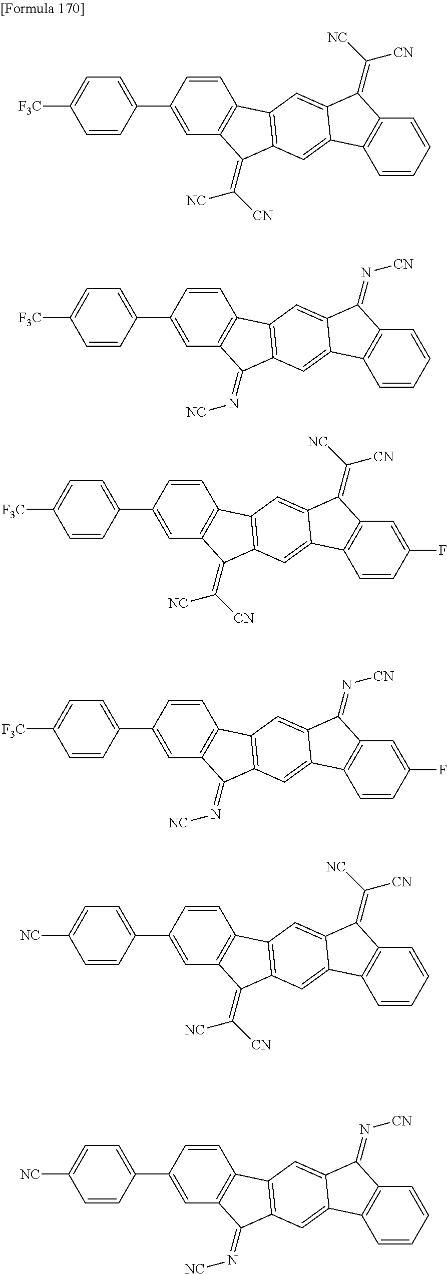 [SMILES: N#CC(C#N)=C1c2ccccc2-c2cc3c(cc21)-c1ccc(-c2ccc(C(F)(F)F)cc2)cc1C3=C(C#N)C#N ; N#C/N=c1\c2ccccc2c2cc3/c(=N/C#N)c4cc(-c5ccc(C(F)(F)F)cc5)ccc4c3cc12 ; N#CC(C#N)=C1c2cc(F)ccc2-c2cc3c(cc21)-c1ccc(-c2ccc(C(F)(F)F)cc2)cc1C3=C(C#N)C#N ; N#C/N=c1\c2cc(F)ccc2c2cc3/c(=N/C#N)c4cc(-c5ccc(C(F)(F)F)cc5)ccc4c3cc12 ; N#CC(C#N)=C1c2ccccc2-c2cc3c(cc21)-c1ccc(-c2ccc(C#N)cc2)cc1C3=C(C#N)C#N ; N#C/N=c1\c2ccccc2c2cc3/c(=N/C#N)c4cc(-c5ccc(C#N)cc5)ccc4c3cc12]

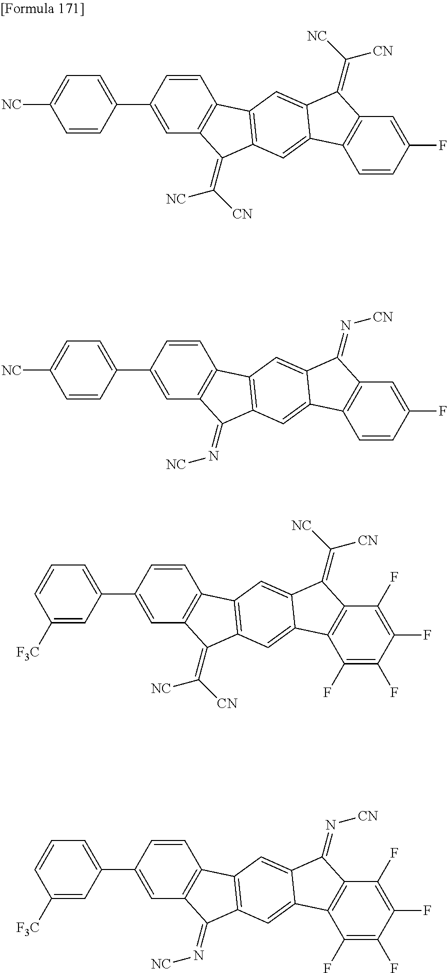 [SMILES: N#CC(C#N)=C1c2cc(F)ccc2-c2cc3c(cc21)-c1ccc(-c2ccc(C#N)cc2)cc1C3=C(C#N)C#N ; N#C/N=c1\c2cc(F)ccc2c2cc3/c(=N/C#N)c4cc(-c5ccc(C#N)cc5)ccc4c3cc12 ; N#CC(C#N)=C1c2cc(-c3cccc(C(F)(F)F)c3)ccc2-c2cc3c(cc21)-c1c(F)c(F)c(F)c(F)c1C3=C(C#N)C#N ; N#C/N=c1/c2cc3c(cc2c2c(F)c(F)c(F)c(F)c12)/c(=N/C#N)c1cc(-c2cccc(C(F)(F)F)c2)ccc13]

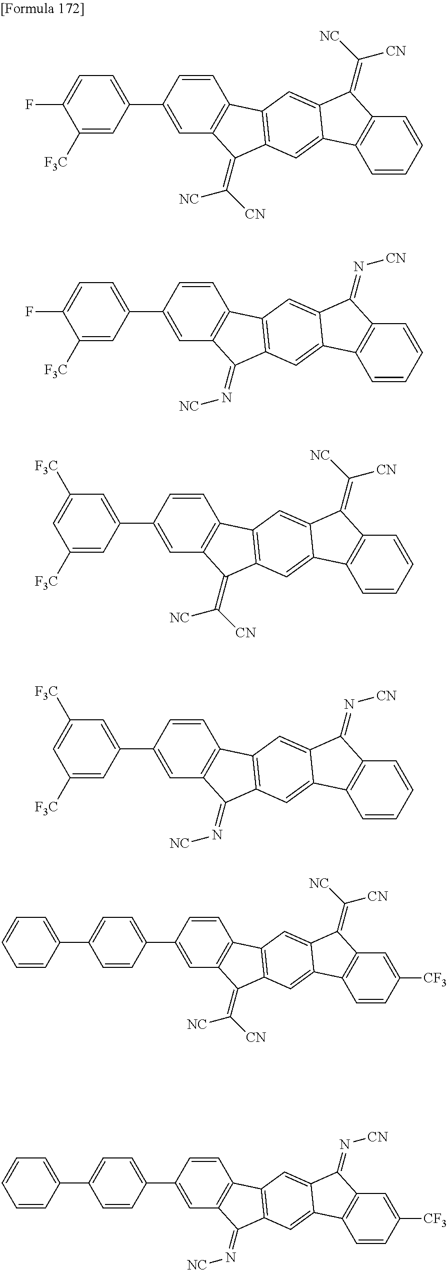 [SMILES: N#CC(C#N)=C1c2ccccc2-c2cc3c(cc21)-c1ccc(-c2ccc(F)c(C(F)(F)F)c2)cc1C3=C(C#N)C#N ; N#C/N=c1\c2ccccc2c2cc3/c(=N/C#N)c4cc(-c5ccc(F)c(C(F)(F)F)c5)ccc4c3cc12 ; N#CC(C#N)=C1c2ccccc2-c2cc3c(cc21)-c1ccc(-c2cc(C(F)(F)F)cc(C(F)(F)F)c2)cc1C3=C(C#N)C#N ; N#C/N=c1\c2ccccc2c2cc3/c(=N/C#N)c4cc(-c5cc(C(F)(F)F)cc(C(F)(F)F)c5)ccc4c3cc12 ; N#CC(C#N)=C1c2cc(-c3ccc(-c4ccccc4)cc3)ccc2-c2cc3c(cc21)-c1ccc(C(F)(F)F)cc1C3=C(C#N)C#N ; N#C/N=c1\c2cc(-c3ccc(-c4ccccc4)cc3)ccc2c2cc3/c(=N/C#N)c4cc(C(F)(F)F)ccc4c3cc12]

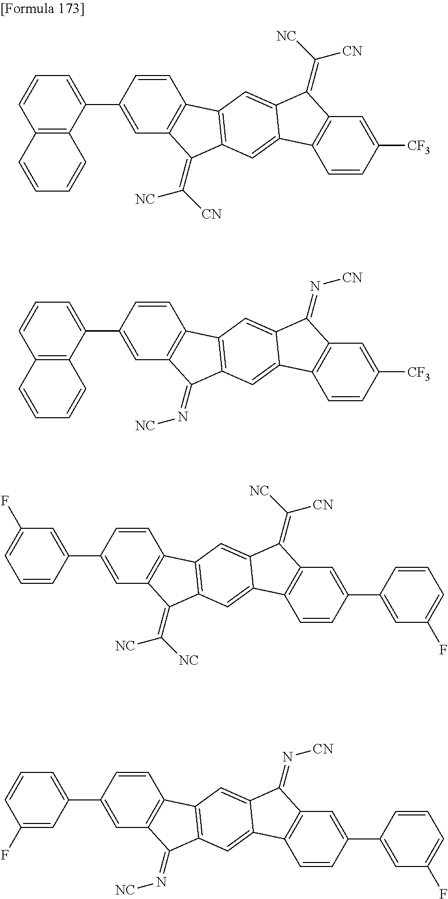

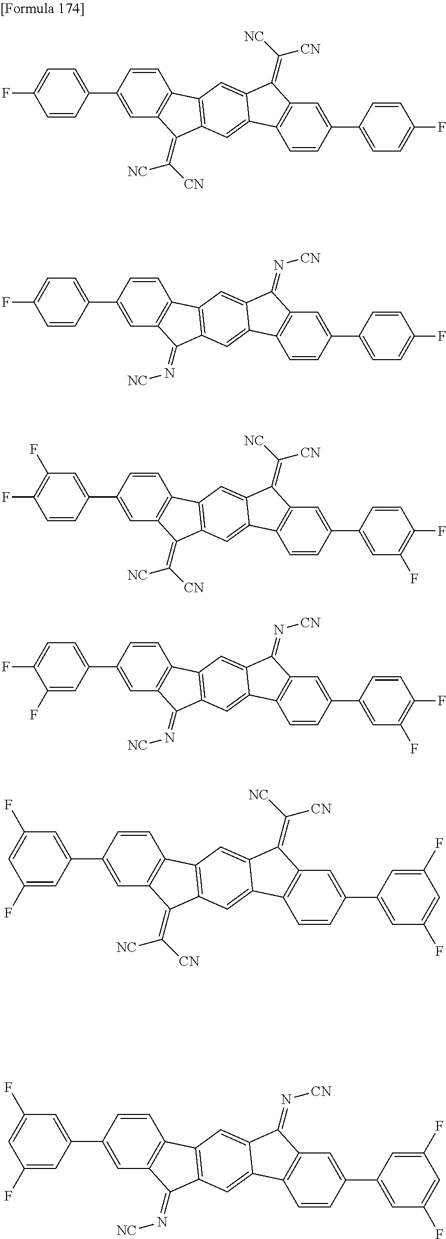

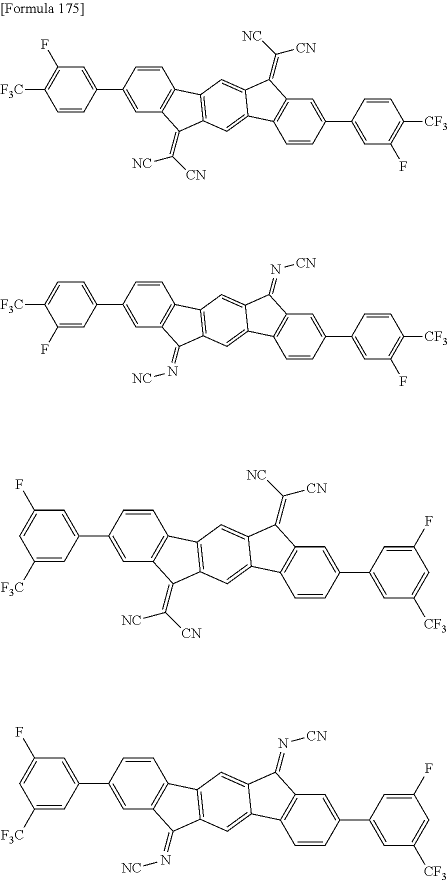 [SMILES: N#CC(C#N)=C1c2cc(-c3ccc(C(F)(F)F)c(F)c3)ccc2-c2cc3c(cc21)-c1ccc(-c2ccc(C(F)(F)F)c(F)c2)cc1C3=C(C#N)C#N ; N#C/N=c1\c2cc(-c3ccc(C(F)(F)F)c(F)c3)ccc2c2cc3/c(=N/C#N)c4cc(-c5ccc(C(F)(F)F)c(F)c5)ccc4c3cc12 ; N#CC(C#N)=C1c2cc(-c3cc(F)cc(C(F)(F)F)c3)ccc2-c2cc3c(cc21)-c1ccc(-c2cc(F)cc(C(F)(F)F)c2)cc1C3=C(C#N)C#N ; N#C/N=c1\c2cc(-c3cc(F)cc(C(F)(F)F)c3)ccc2c2cc3/c(=N/C#N)c4cc(-c5cc(F)cc(C(F)(F)F)c5)ccc4c3cc12]

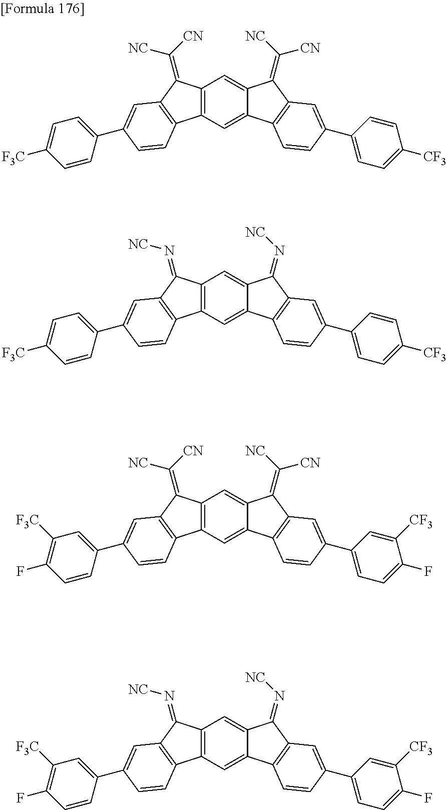 [SMILES: N#CC(C#N)=C1c2cc(-c3ccc(C(F)(F)F)cc3)ccc2-c2cc3c(cc21)C(=C(C#N)C#N)c1cc(-c2ccc(C(F)(F)F)cc2)ccc1-3 ; N#C/N=c1/c2cc(-c3ccc(C(F)(F)F)cc3)ccc2c2cc3c(cc12)/c(=N/C#N)c1cc(-c2ccc(C(F)(F)F)cc2)ccc13 ; N#CC(C#N)=C1c2cc(-c3ccc(F)c(C(F)(F)F)c3)ccc2-c2cc3c(cc21)C(=C(C#N)C#N)c1cc(-c2ccc(F)c(C(F)(F)F)c2)ccc1-3 ; N#C/N=c1/c2cc(-c3ccc(F)c(C(F)(F)F)c3)ccc2c2cc3c(cc12)/c(=N/C#N)c1cc(-c2ccc(F)c(C(F)(F)F)c2)ccc13]

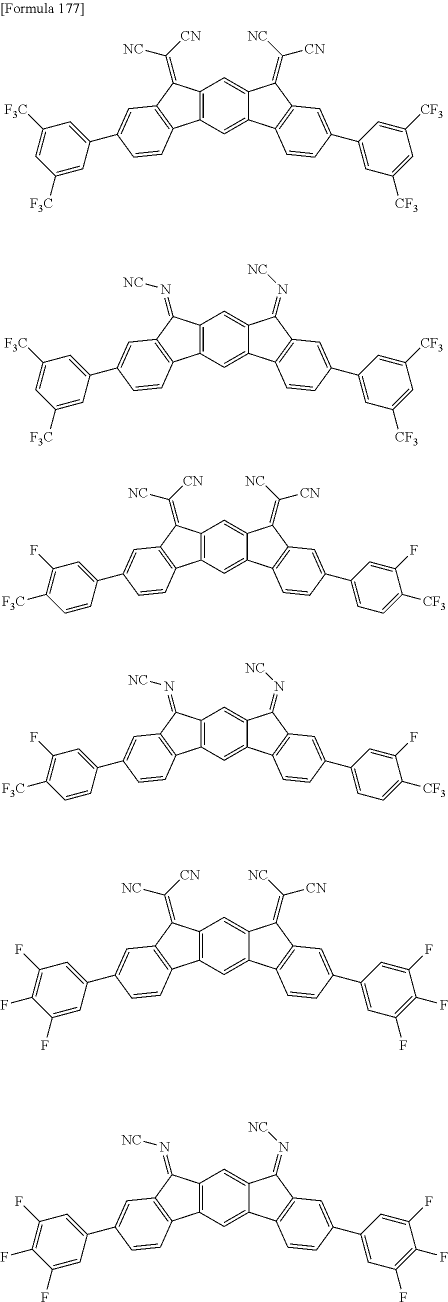

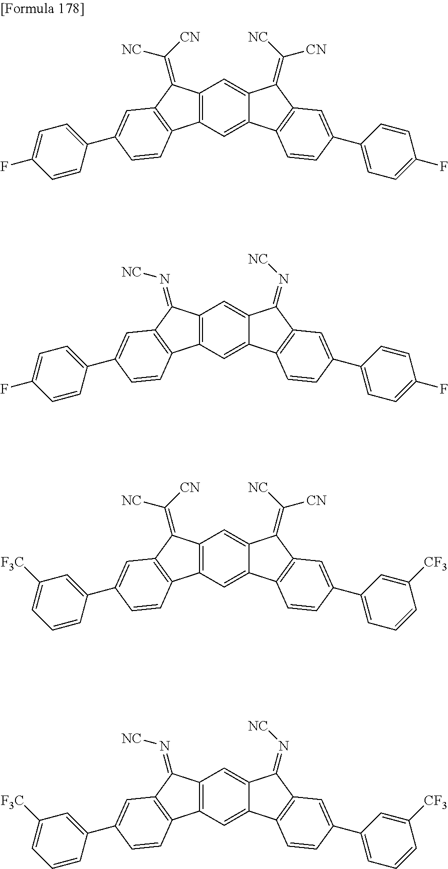 [SMILES: N#CC(C#N)=C1c2cc(-c3ccc(F)cc3)ccc2-c2cc3c(cc21)C(=C(C#N)C#N)c1cc(-c2ccc(F)cc2)ccc1-3 ; N#C/N=c1/c2cc(-c3ccc(F)cc3)ccc2c2cc3c(cc12)/c(=N/C#N)c1cc(-c2ccc(F)cc2)ccc13 ; N#CC(C#N)=C1c2cc(-c3cccc(C(F)(F)F)c3)ccc2-c2cc3c(cc21)C(=C(C#N)C#N)c1cc(-c2cccc(C(F)(F)F)c2)ccc1-3 ; N#C/N=c1/c2cc(-c3cccc(C(F)(F)F)c3)ccc2c2cc3c(cc12)/c(=N/C#N)c1cc(-c2cccc(C(F)(F)F)c2)ccc13]

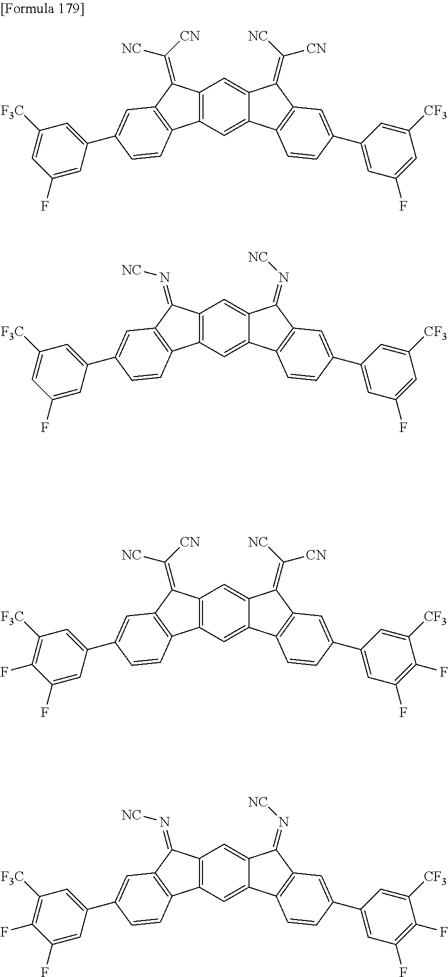 [SMILES: N#CC(C#N)=C1c2cc(-c3cc(F)cc(C(F)(F)F)c3)ccc2-c2cc3c(cc21)C(=C(C#N)C#N)c1cc(-c2cc(F)cc(C(F)(F)F)c2)ccc1-3 ; N#C/N=c1/c2cc(-c3cc(F)cc(C(F)(F)F)c3)ccc2c2cc3c(cc12)/c(=N/C#N)c1cc(-c2cc(F)cc(C(F)(F)F)c2)ccc13 ; N#CC(C#N)=C1c2cc(-c3cc(F)c(F)c(C(F)(F)F)c3)ccc2-c2cc3c(cc21)C(=C(C#N)C#N)c1cc(-c2cc(F)c(F)c(C(F)(F)F)c2)ccc1-3 ; N#C/N=c1/c2cc(-c3cc(F)c(F)c(C(F)(F)F)c3)ccc2c2cc3c(cc12)/c(=N/C#N)c1cc(-c2cc(F)c(F)c(C(F)(F)F)c2)ccc13]

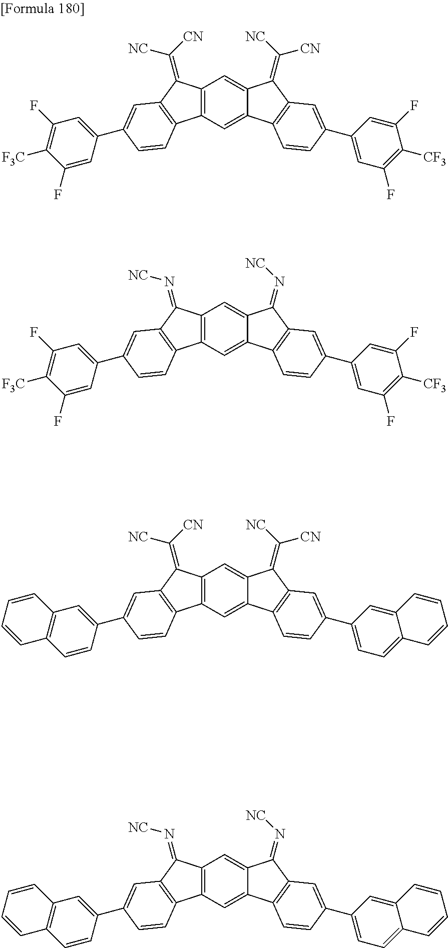 [SMILES: N#CC(C#N)=C1c2cc(-c3cc(F)c(C(F)(F)F)c(F)c3)ccc2-c2cc3c(cc21)C(=C(C#N)C#N)c1cc(-c2cc(F)c(C(F)(F)F)c(F)c2)ccc1-3 ; N#C/N=c1/c2cc(-c3cc(F)c(C(F)(F)F)c(F)c3)ccc2c2cc3c(cc12)/c(=N/C#N)c1cc(-c2cc(F)c(C(F)(F)F)c(F)c2)ccc13 ; N#CC(C#N)=C1c2cc(-c3ccc4ccccc4c3)ccc2-c2cc3c(cc21)C(=C(C#N)C#N)c1cc(-c2ccc4ccccc4c2)ccc1-3 ; N#C/N=c1/c2cc(-c3ccc4ccccc4c3)ccc2c2cc3c(cc12)/c(=N/C#N)c1cc(-c2ccc4ccccc4c2)ccc13]

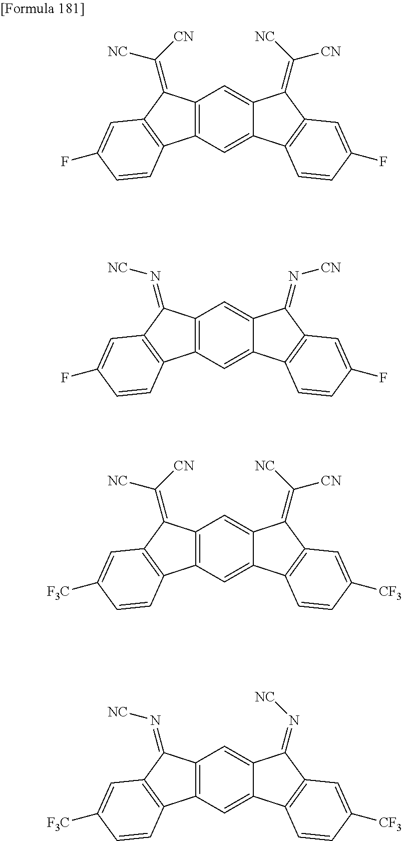 [SMILES: N#CC(C#N)=C1c2cc(F)ccc2-c2cc3c(cc21)C(=C(C#N)C#N)c1cc(F)ccc1-3 ; N#C/N=c1\c2cc(F)ccc2c2cc3c(cc12)/c(=N/C#N)c1cc(F)ccc13 ; N#CC(C#N)=C1c2cc(C(F)(F)F)ccc2-c2cc3c(cc21)C(=C(C#N)C#N)c1cc(C(F)(F)F)ccc1-3 ; N#C/N=c1/c2cc(C(F)(F)F)ccc2c2cc3c(cc12)/c(=N/C#N)c1cc(C(F)(F)F)ccc13]

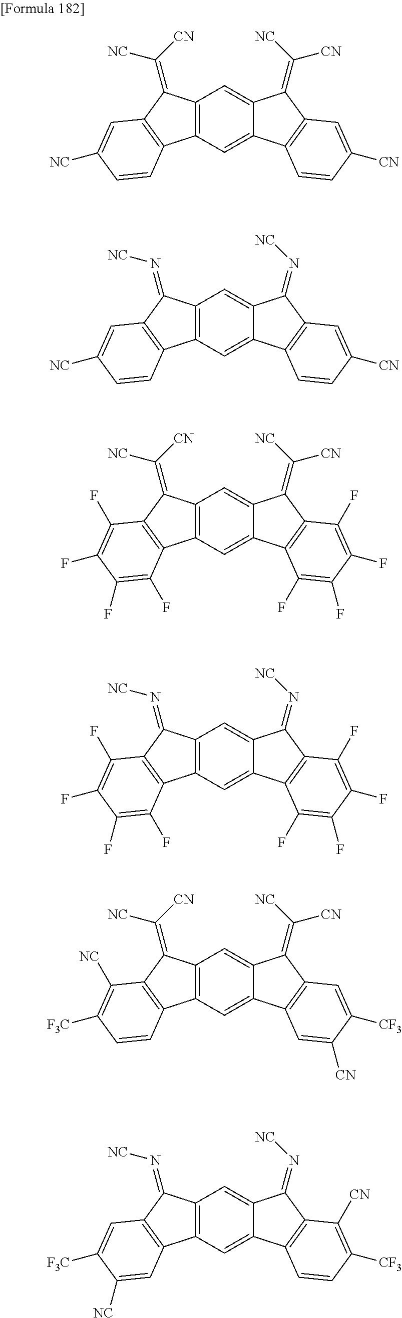 [SMILES: N#CC(C#N)=C1c2cc(C#N)ccc2-c2cc3c(cc21)C(=C(C#N)C#N)c1cc(C#N)ccc1-3 ; N#C/N=c1/c2cc(C#N)ccc2c2cc3c(cc12)/c(=N/C#N)c1cc(C#N)ccc13 ; N#CC(C#N)=C1c2cc3c(cc2-c2c(F)c(F)c(F)c(F)c21)-c1c(F)c(F)c(F)c(F)c1C3=C(C#N)C#N ; N#C/N=c1/c2cc3/c(=N\C#N)c4c(F)c(F)c(F)c(F)c4c3cc2c2c(F)c(F)c(F)c(F)c12 ; N#CC(C#N)=C1c2cc(C(F)(F)F)c(C#N)cc2-c2cc3c(cc21)C(=C(C#N)C#N)c1c-3ccc(C(F)(F)F)c1C#N ; N#C/N=c1\c2cc(C(F)(F)F)c(C#N)cc2c2cc3c(cc12)/c(=N\C#N)c1c(C#N)c(C(F)(F)F)ccc13]

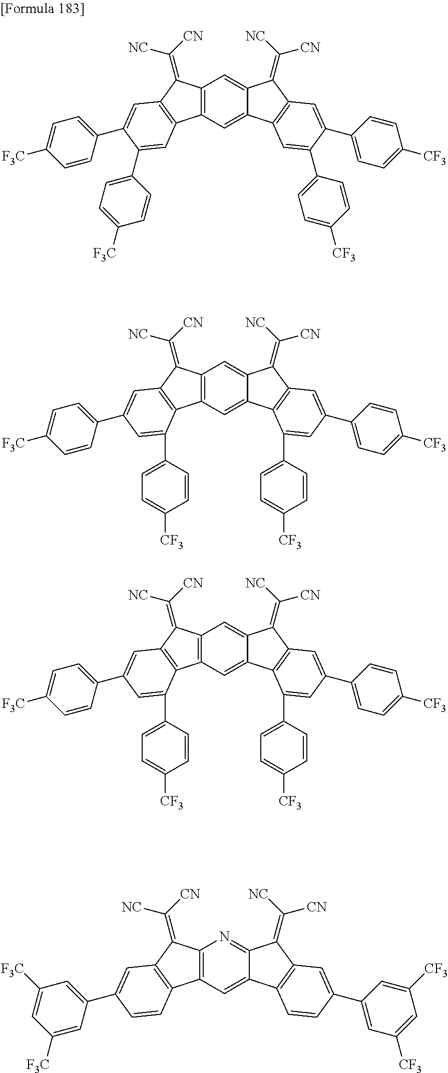

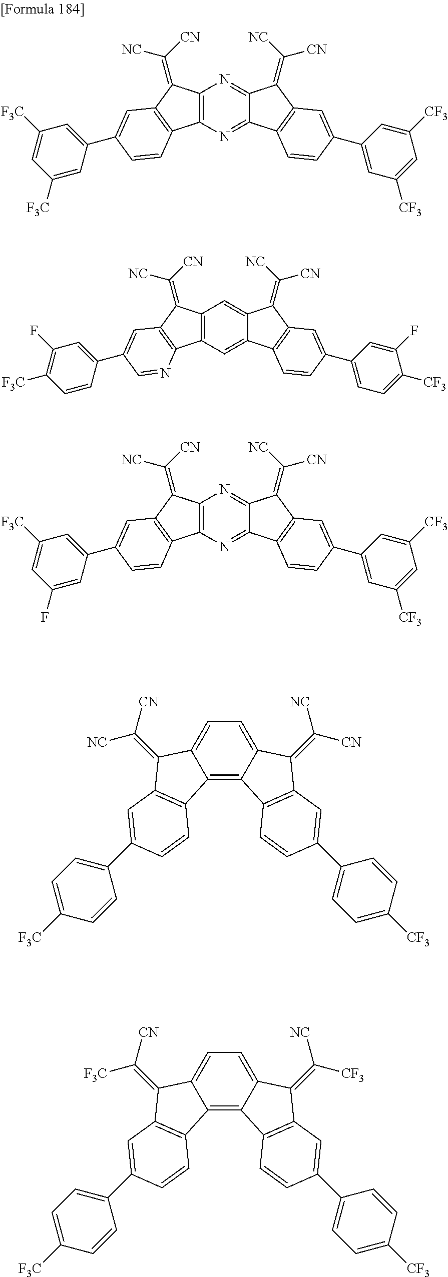

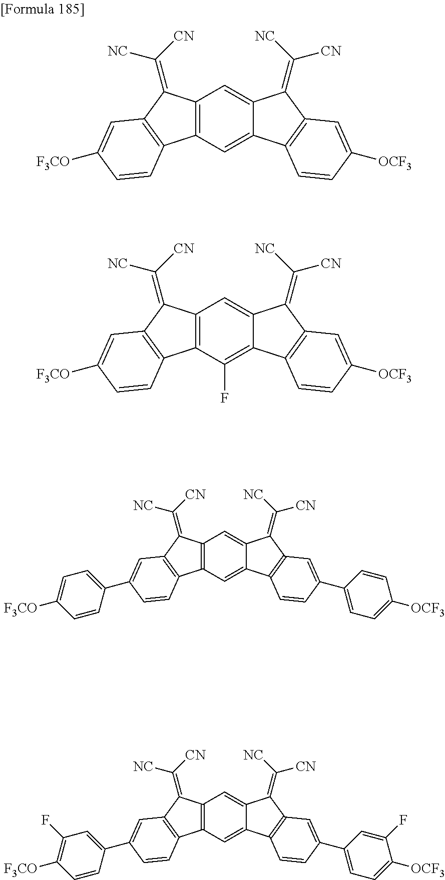 [SMILES: N#CC(C#N)=C1c2cc(OC(F)(F)F)ccc2-c2cc3c(cc21)C(=C(C#N)C#N)c1cc(OC(F)(F)F)ccc1-3 ; N#CC(C#N)=C1c2cc(OC(F)(F)F)ccc2-c2c1cc1c(c2F)-c2ccc(OC(F)(F)F)cc2C1=C(C#N)C#N ; N#CC(C#N)=C1c2cc(-c3ccc(OC(F)(F)F)cc3)ccc2-c2cc3c(cc21)C(=C(C#N)C#N)c1cc(-c2ccc(OC(F)(F)F)cc2)ccc1-3 ; N#CC(C#N)=C1c2cc(-c3ccc(OC(F)(F)F)c(F)c3)ccc2-c2cc3c(cc21)C(=C(C#N)C#N)c1cc(-c2ccc(OC(F)(F)F)c(F)c2)ccc1-3]

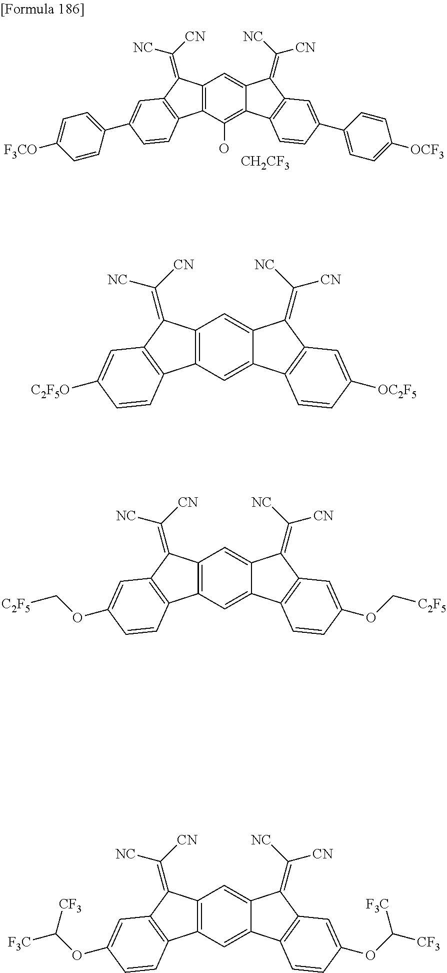

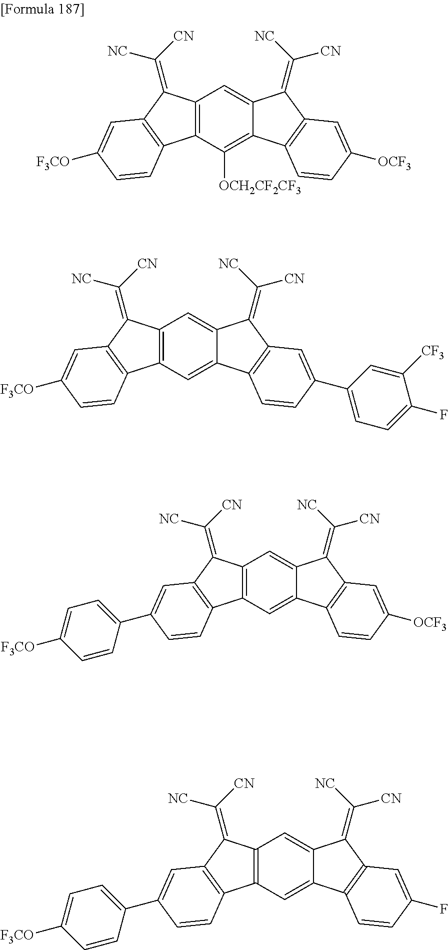

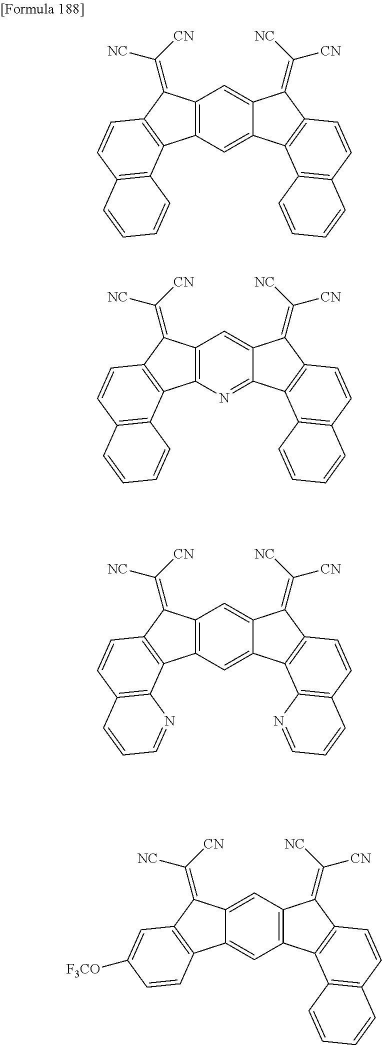 [SMILES: N#CC(C#N)=C1c2cc3c(cc2-c2c1ccc1ccccc21)-c1c(ccc2ccccc12)C3=C(C#N)C#N ; N#CC(C#N)=C1c2cc3c(nc2-c2c1ccc1ccccc21)-c1c(ccc2ccccc12)C3=C(C#N)C#N ; N#CC(C#N)=C1c2cc3c(cc2-c2c1ccc1cccnc21)-c1c(ccc2cccnc12)C3=C(C#N)C#N ; N#CC(C#N)=C1c2cc(OC(F)(F)F)ccc2-c2cc3c(cc21)C(=C(C#N)C#N)c1ccc2ccccc2c1-3]

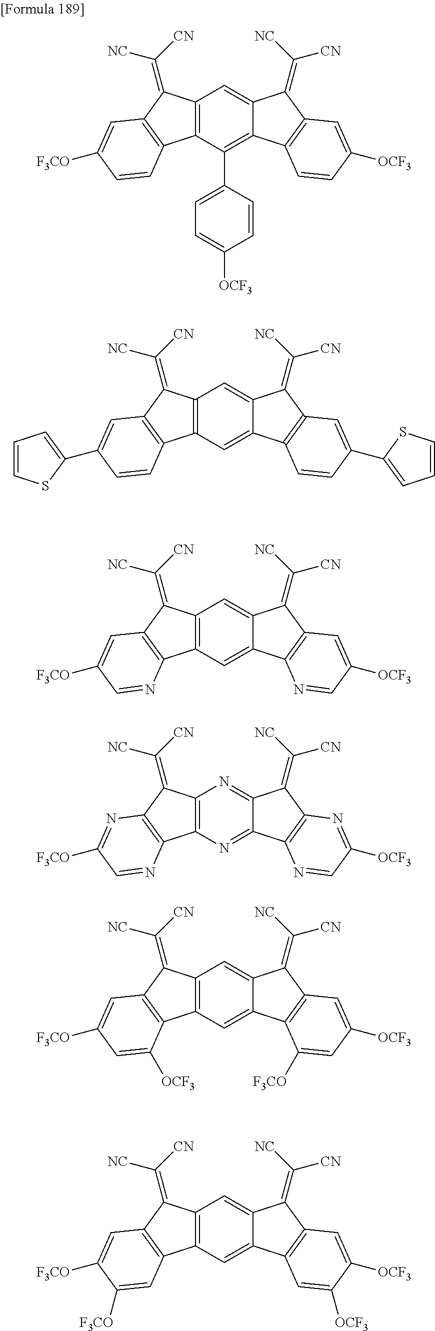

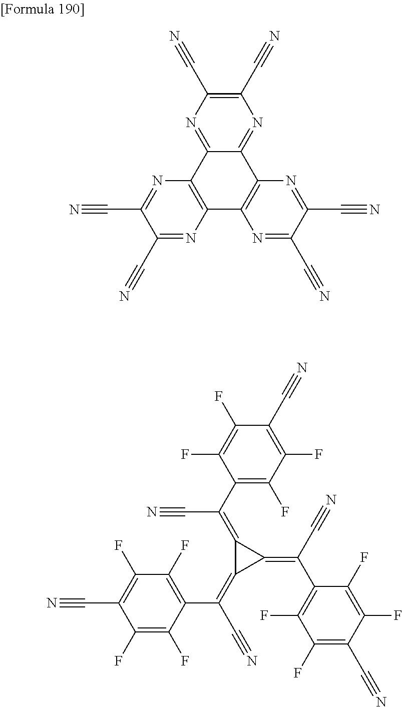 [SMILES: N#Cc1nc2c3nc(C#N)c(C#N)nc3c3nc(C#N)c(C#N)nc3c2nc1C#N ; N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F]

### Emitting Region

The emitting region includes one or more emitting layers. The one or more emitting layers include the first emitting layer.

### First Emitting Layer

The first emitting layer contains the first host material, the first additional host material different from the first host material, and the first luminescent compound. Although the first host material is not particularly limited, for instance, a compound selected from the group consisting of a compound represented by a formula (H10) described below and a first compound described below is usable. Although the first additional host material is not particularly limited, for instance, a compound represented by a formula (H20) described below is usable.

Although the first luminescent compound is not particularly limited, for instance, a compound selected from the group consisting of a compound represented by a formula (6) described below, a third compound described below, and a fourth compound described below is usable.

The first luminescent compound is preferably a compound that emits light having a maximum peak wavelength of 500 nm or less, more preferably a compound that emits light having a maximum peak wavelength in a range from 430 nm to 480 nm. The first luminescent compound is preferably a fluorescent compound that emits fluorescence having a maximum peak wavelength of 500 nm or less, more preferably a fluorescent compound that emits fluorescence having a maximum peak wavelength in a range from 430 nm to 480 nm.

In an exemplary arrangement of the organic EL device according to the exemplary embodiment, the full width at half maximum of the maximum peak of the first luminescent compound is in a range from 1 nm to 30 nm.

In an exemplary arrangement of the organic EL device according to the exemplary embodiment, the first luminescent compound is a compound containing no azine ring structure in a molecule.

In an exemplary arrangement of the organic EL device according to the exemplary embodiment, the first luminescent compound is preferably not a boron-containing complex, more preferably not a complex.

As a fluorescent compound that emits blue fluorescence and is usable for the first emitting layer, for instance, compounds such as a pyrene derivative, a styrylamine derivative, a chrysene derivative, a fluoranthene derivative, a fluorene derivative, a diamine derivative, and a triarylamine derivative may be used.

Herein, the blue light emission refers to a light emission in which a maximum peak wavelength of emission spectrum is in a range from 430 nm to 500 nm.

In an exemplary arrangement of the organic EL device of the exemplary embodiment, when the emitting region includes two or more emitting layers, the two or more emitting layers are each an emitting layer that emits fluorescence.

In an exemplary arrangement of the organic EL device according to the exemplary embodiment, the first emitting layer contains no metal complex. In an exemplary arrangement of the organic EL device according to the exemplary embodiment, the first emitting layer contains no boron-containing complex.

In an exemplary arrangement of the organic EL device according to the exemplary embodiment, the first emitting layer contains no phosphorescent material (dopant material).

In an exemplary arrangement of the organic EL device according to the exemplary embodiment, the first emitting layer contains no heavy-metal complex and no phosphorescent rare earth metal complex. Examples of the heavy-metal complex herein include an iridium complex, osmium complex, and platinum complex.

A method of measuring the maximum peak wavelength of the compound is as follows. A toluene solution of a measurement target compound at a concentration of 5 μmol/L was prepared and put in a quartz cell. An emission spectrum (ordinate axis: luminous intensity, abscissa axis: wavelength) of the thus-obtained sample was measured at a normal temperature (300K). The emission spectrum can be measured using a spectrophotometer (apparatus name: F-7000) produced by Hitachi High-Tech Science Corporation. It should be noted that the apparatus for measuring the emission spectrum is not limited to the apparatus used herein.

A peak wavelength of the emission spectrum exhibiting the maximum luminous intensity is defined as the maximum peak wavelength. Herein, the maximum peak wavelength of fluorescence is occasionally referred to as a maximum fluorescence peak wavelength (FL-peak).

In an emission spectrum of the first luminescent compound, where a peak exhibiting a maximum luminous intensity is defined as a maximum peak and a height of the maximum peak is defined as 1, heights of other peaks appearing in the emission spectrum are preferably less than 0.6. It should be noted that the peaks in the emission spectrum are defined as local maximum values.

Moreover, in the emission spectrum of the first luminescent compound, the number of peaks is preferably less than three.

In an exemplary arrangement of the organic EL device according to the exemplary embodiment, the first emitting layer contains 0.5 mass % or more of the first luminescent compound with respect to a total mass of the first emitting layer.

The first emitting layer contains the first luminescent compound preferably at 10 mass % or less, more preferably at 7 mass % or less, and still more preferably at 5 mass % less, with respect to the total mass of the first emitting layer.

Herein, the “host material” refers to, for instance, a material that accounts for “50 mass % or more of the layer”.

In an exemplary arrangement of the organic EL device according to the exemplary embodiment, the first emitting layer preferably contains the first host material and the first additional host material in total, at 60 mass % or more, more preferably at 70 mass % or more, still more preferably at 80 mass % or more, still further more preferably at 90 mass % or more, and yet still further more preferably at 95 mass % or more, with respect to the total mass of the first emitting layer.

The first emitting layer preferably contains the first host material and the first additional host material in total at 99 mass % or less with respect to the total mass of the first emitting layer.

The upper limit of the total of the content ratios of the first host material, the first additional host material, and the first luminescent compound in the first emitting layer is 100 mass %.

In the organic EL device according to the exemplary embodiment, the first emitting layer may further contain any other material than the first host material, the first additional host material, and the first luminescent compound.

The first emitting layer may contain a single type of the first host material or may contain two or more types of the first host material.

The first emitting layer may contain a single type of the first additional host material or may contain two or more types of the first additional host material. The first emitting layer may contain a single type of the first luminescent compound or may contain two or more types of the first luminescent compound.

### First Host Material

In an exemplary arrangement of the organic EL device according to the exemplary embodiment, the first host material has at least one deuterium atom.

In an exemplary arrangement of the organic EL device according to the exemplary embodiment, the first host material has no deuterium atom.

In an exemplary arrangement of the organic EL device according to the exemplary embodiment, the first host material is a compound represented by the formula (H10) below.

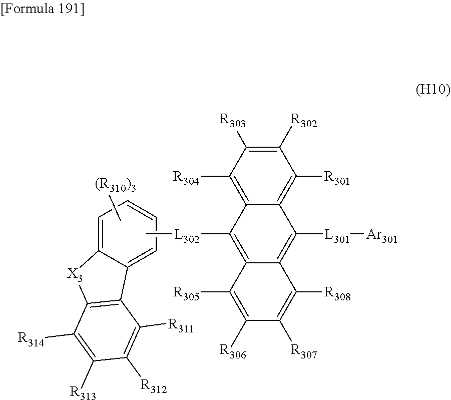

In the formula (H10):
X₃ is an oxygen atom or a sulfur atom;
at least one combination of adjacent two or more of three R₃₁₀ are mutually bonded to form a substituted or unsubstituted monocyclic ring, mutually bonded to form a substituted or unsubstituted fused ring, or not mutually bonded;
at least one combination of adjacent two or more of R₃₁₁ to R₃₁₄ are mutually bonded to form a substituted or unsubstituted monocyclic ring, or mutually bonded to form a substituted or unsubstituted fused ring;
R₃₀₁ to R₃₀₈, and R₃₁₀ to R₃₁₄ forming neither the substituted or unsubstituted monocyclic ring nor the substituted or unsubstituted fused ring are each a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms;
L₃₀₁ and L₃₀₂ are each independently a single bond, a substituted or unsubstituted arylene group having 6 to 50 ring carbon atoms, or a substituted or unsubstituted divalent heterocyclic group having 5 to 50 ring atoms;
Ar₃₀₁ is a substituted or unsubstituted aryl group having 6 to 50 ring carbon atoms, or a substituted or unsubstituted heterocyclic group having 5 to 50 ring atoms. In the first host material, R₉₀₁, R₉₀₂, R₉₀₃, R₉₀₄, R₉₀₅, R₉₀₆ and R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different; and
when a plurality of R₉₀₇ are present, the plurality of R₉₀₇ are mutually the same or different.

In an exemplary arrangement of the organic EL device according to the exemplary embodiment, at least one combination of adjacent two or more of R₃₁₁ to R₃₁₄ in the formula (H10) are mutually bonded to form a substituted or unsubstituted benzene ring.

In an exemplary arrangement of the organic EL device according to the exemplary embodiment, R₃₀₁ to R₃₀₈ each independently have at least one deuterium atom.

In an exemplary arrangement of the organic EL device according to the exemplary embodiment, L₃₀₁, L₃₀₂, Ar₃₀₁, and R₃₁₀ to R₃₁₄ each independently have at least one deuterium atom.

In an exemplary arrangement of the organic EL device according to the exemplary embodiment, the first host material is a compound represented by a formula (H31), (H32) or (H33) below.

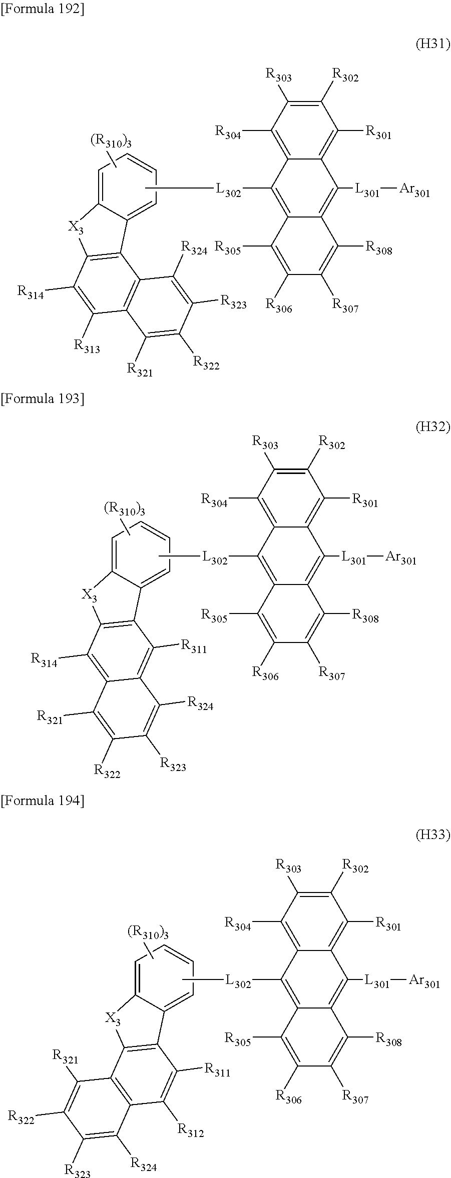

In the formulae (H31), (H32) and (H33), X₃, R₃₀₁ to R₃₀₈, R₃₁₀ to R₃₁₄, L₃₀₁, L₃₀₂, and Ar₃₀₁ each independently represent the same as X₃, R₃₀₁ to R₃₀₈, R₃₁₀ to R₃₁₄, L₃₀₁, L₃₀₂, and Ar₃₀₁ in the formula (H10); and
R₃₂₁ to R₃₂₄ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In an exemplary arrangement of the organic EL device according to the exemplary embodiment, the first host material is a compound represented by a formula (H301) or (H302) below.

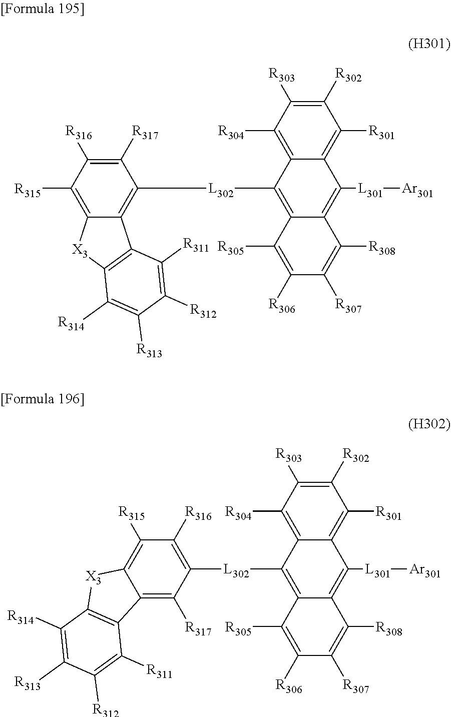

In the formulae (H301) and (H302), X₃, R₃₀₁ to R₃₀, R₃₁₁ to R₃₁₄, L₃₀₁, L₃₀₂, and Ar₃₀₁ each independently represent the same as X₃, R₃₀₁ to R₃₀, R₃₁₁ to R₃₁₄, L₃₀₁, L₃₀₂, and Ar₃₀₁ in the formula (H10);
at least one combination of adjacent two or more of R₃₁₅ to R₃₁₇ are mutually bonded to form a substituted or unsubstituted monocyclic ring, mutually bonded to form a substituted or unsubstituted fused ring, or not mutually bonded; and
R₃₁₅ to R₃₁₇ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In an exemplary arrangement of the organic EL device according to the exemplary embodiment, the first host material is a compound represented by a formula (H311), (H312), (H321), (H322), (H331), or (H332) below.

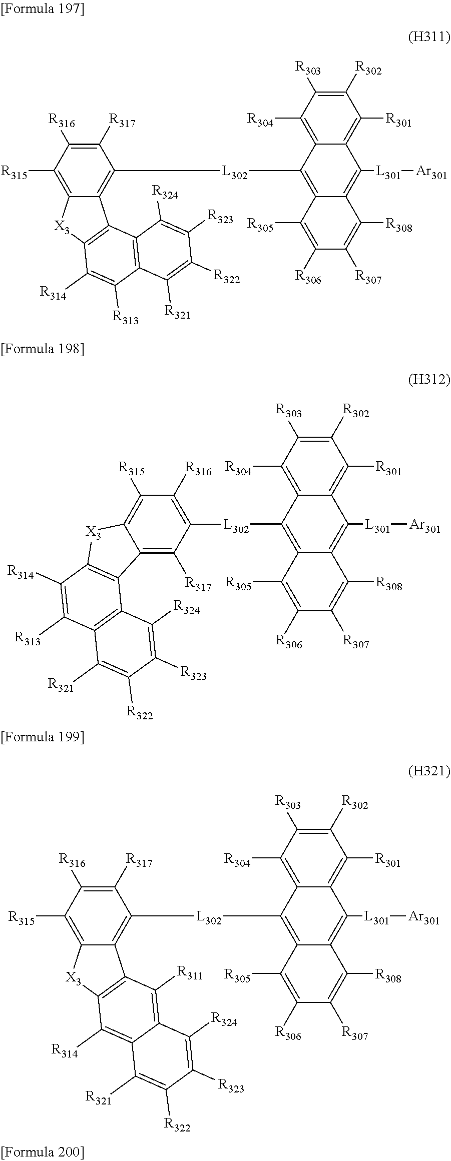

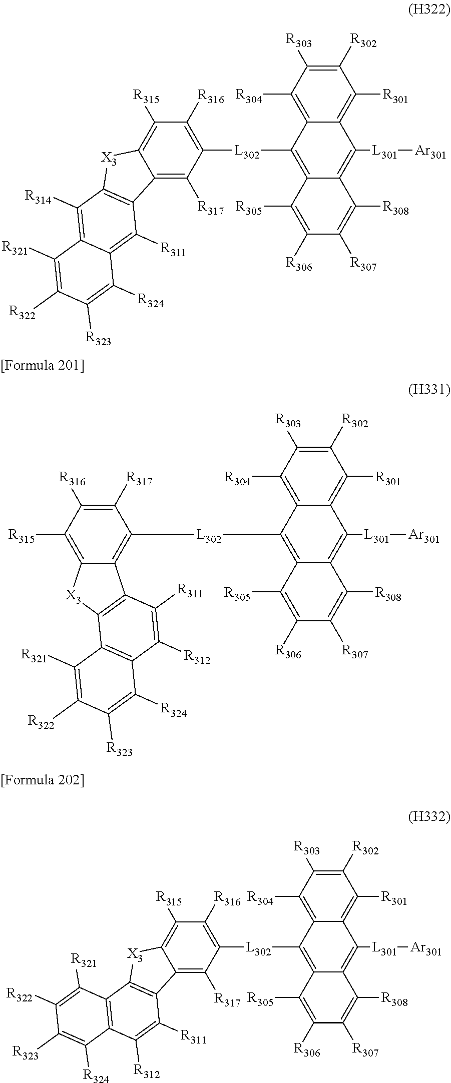

In the formulae (H311), (H312), (H321), (H322), (H331) and (H332), X₃, R₃₀₁ to R₃₀₈, L₃₀₁, L₃₀₂, and Ar₃₀₁ each independently represent the same as X₃, R₃₀₁ to R₃₀₈, L₃₀₁, L₃₀₂, and Ar₃₀₁ in the formula (H10); and
R₃₁₁ to R₃₁₇ and R₃₂₁ to R₃₂₄ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In an exemplary arrangement of the organic EL device according to the exemplary embodiment, L₃₀₁ and L₃₀₂ of the first host material are each independently a single bond or a substituted or unsubstituted arylene group having 6 to 14 ring carbon atoms.

In an exemplary arrangement of the organic EL device according to the exemplary embodiment, L₃₀₁ and L₃₀₂ of the first host material are each independently a single bond, a substituted or unsubstituted phenylene group or a substituted or unsubstituted naphthylene group.

In an exemplary arrangement of the organic EL device of the exemplary embodiment, L₃₀₁ and L₃₀₂ of the first host material are each a single bond.

In an exemplary arrangement of the organic EL device according to the exemplary embodiment, the first host material is a compound represented by a formula (H313), (H314), (H323), (H324), (H333), or (H334) below.

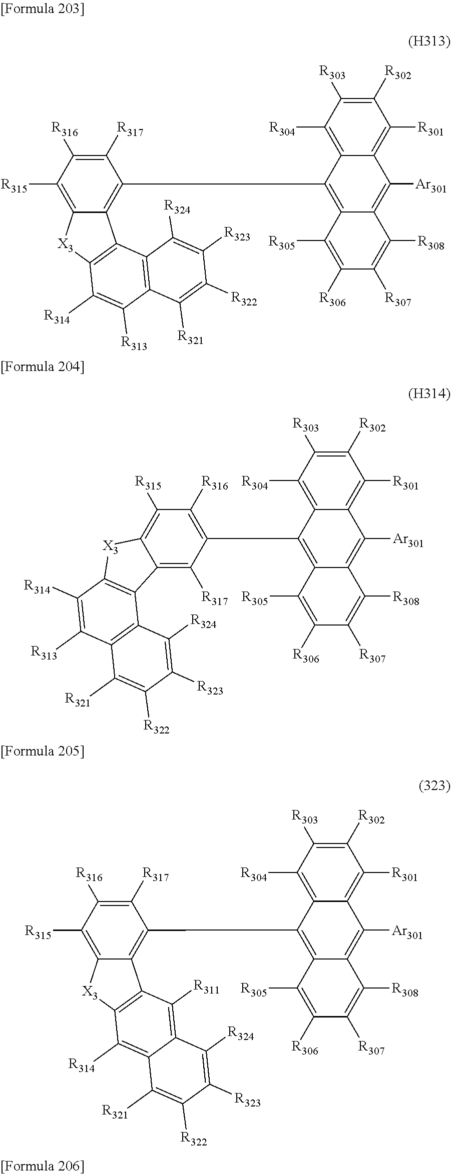

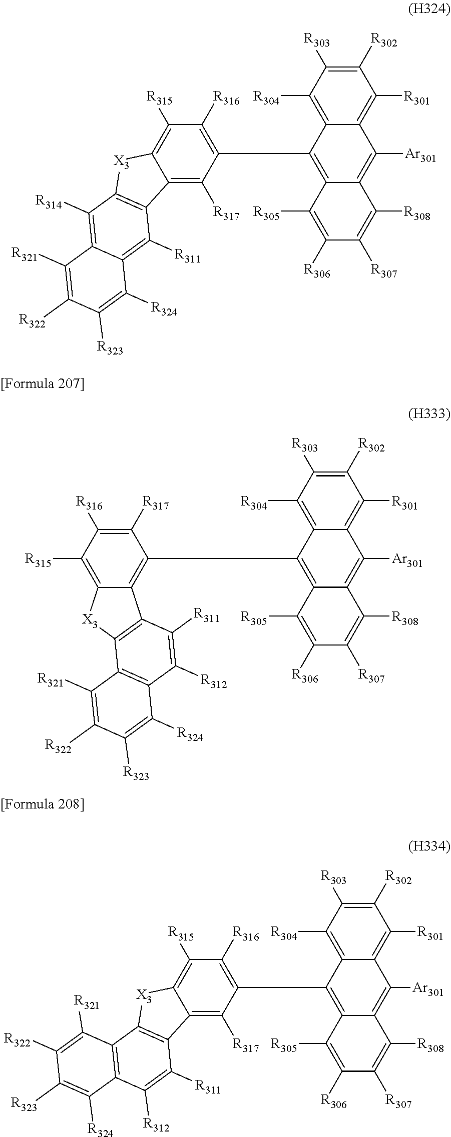

In the formulae (H313), (H314), (H323), (H324), (H333) and (H334), X₃, R₃₀₁ to R₃₀₈, and Ar₃₀₁ each independently represent the same as X₃, R₃₀₁ to R₃₀₈, and Ar₃₀₁ in the formula (H10), and
R₃₁₁ to R₃₁₇ and R₃₂₁ to R₃₂₄ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In an exemplary arrangement of the organic EL device of the exemplary embodiment, R₃₁₁ to R₃₁₇ and R₃₂₁ to R₃₂₄ of the first host material are each independently a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms.

In an exemplary arrangement of the organic EL device of the exemplary embodiment, R₃₁₁ to R₃₁₇ and R₃₂₁ to R₃₂₄ of the first host material are each independently a hydrogen atom, a substituted or unsubstituted alkyl group having 1 to 50 carbon atoms, or a substituted or unsubstituted aryl group having 6 to 50 ring carbon atoms.

In an exemplary arrangement of the organic EL device according to the exemplary embodiment, Ar₃₀₁ of the first host material is a substituted or unsubstituted aryl group having 6 to 50 ring carbon atoms.

In an exemplary arrangement of the organic EL device according to the exemplary embodiment, Ar₃₀₁ of the first host material is a group represented by a formula (a1), (a2), (a3) or (a4) below.

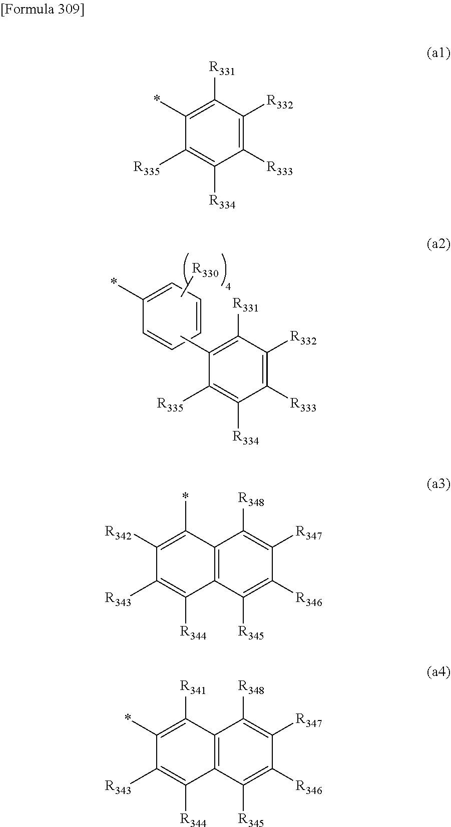

In the formulae (a1), (a2), (a3) and (a4):
at least one combination of adjacent two or more of R₃₃₀ to R₃₃₅ are mutually bonded to form a substituted or unsubstituted monocyclic ring, mutually bonded to form a substituted or unsubstituted fused ring, or not mutually bonded;
at least one combination of adjacent two or more of R₃₄₁ to R₃₄₈ are mutually bonded to form a substituted or unsubstituted monocyclic ring, mutually bonded to form a substituted or unsubstituted fused ring, or not mutually bonded;
R₃₃₀ to R₃₃₅ and R₃₄₁ to R₃₄₈ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms;
a plurality of R₃₃₀ are mutually the same or different; and
* each represent a bonding position to L₃₀₁.

In an exemplary arrangement of the organic EL device according to the exemplary embodiment, Ar₃₀₁ of the first host material is a group represented by the formula (a1) or (a2).

In an exemplary arrangement of the organic EL device according to the exemplary embodiment, R₃₃₀ to R₃₃₅ and R₃₄₁ to R₃₄₈ of the first host material are each a hydrogen atom.

In an exemplary arrangement of the organic EL device of the exemplary embodiment, X₃ of the first host material is an oxygen atom.

In an exemplary arrangement of the organic EL device of the exemplary embodiment, R₃₀₁ to R₃₀₈ of the first host material are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, or a substituted or unsubstituted aryl group having 6 to 50 ring carbon atoms.

In an exemplary arrangement of the organic EL device of the exemplary embodiment, R₃₀₁ to R₃₀₈ of the first host material are each independently a hydrogen atom, a substituted or unsubstituted alkyl group having 1 to 50 carbon atoms, or a substituted or unsubstituted aryl group having 6 to 50 ring carbon atoms.

In an exemplary arrangement of the organic EL device of the exemplary embodiment, R₃₀₁ to R₃₀₈ of the first host material are each a hydrogen atom.

### First Additional Host Material

In an exemplary arrangement of the organic EL device according to the exemplary embodiment, the first additional host material has at least one deuterium atom.

In an exemplary arrangement of the organic EL device according to the exemplary embodiment, the first additional host material has no deuterium atom.

### Compound Represented by Formula (H20)

In an exemplary arrangement of the organic EL device according to the exemplary embodiment, the first additional host material is a compound represented by a formula (H20) below.

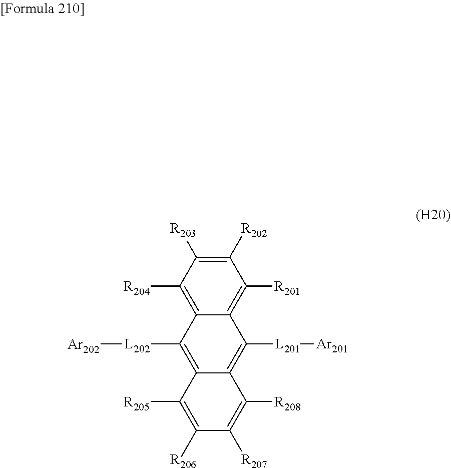

In the formula (H20):
R₂₀₁ to R₂₀₈ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a group represented by a formula (H21) below.

[Formula 211]

-L₂₀₃-Ar₂₀₃ (H21)

In the formulae (H20) and (H21):
L₂₀₁, L₂₀₂, and L₂₀₃ are each independently a single bond, or a substituted or unsubstituted arylene group having 6 to 50 ring carbon atoms; and
Ar₂₀₁, Ar₂₀₂, and Ar₂₀₃ are each independently a substituted or unsubstituted aryl group having 6 to 50 ring carbon atoms, or a substituted or unsubstituted heterocyclic group having 5 to 50 ring atoms.

In the first additional host material, R₉₀₁, R₉₀₂, R₉₀₃, R₉₀₄, R₉₀₅, R₉₀₆ and R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different; and
when a plurality of R₉₀₇ are present, the plurality of R₉₀₇ are mutually the same or different.

In an arrangement of the organic EL device according to the exemplary embodiment, Ar₂₀₁, Ar₂₀₂ and Ar₂₀₃ are each independently a phenyl group, a naphthyl group, a phenanthryl group, a biphenyl group, a terphenyl group, a diphenylfluorenyl group, a dimethylfluorenyl group, a benzodiphenylfluorenyl group, a benzodimethylfluorenyl group, a dibenzofuranyl group, a dibenzothienyl group, a naphthobenzofuranyl group, or a naphthobenzothienyl group.

In an exemplary arrangement of the organic EL device according to the exemplary embodiment, R₂₀₁ to R₂₀₈ each independently have at least one deuterium atom.

In an exemplary arrangement of the organic EL device according to the exemplary embodiment, L₂₀₁, L₂₀₂, L₂₀₃, Ar₂₀₁, Ar₂₀₂ and Ar₂₀₃ each independently have at least one deuterium atom.

In an exemplary arrangement of the organic EL device according to the exemplary embodiment, the compound represented by the formula (H20) is a compound represented by a formula (201), a formula (202), a formula (203), a formula (204), a formula (205), a formula (206), a formula (207), a formula (208), or a formula (209) below.

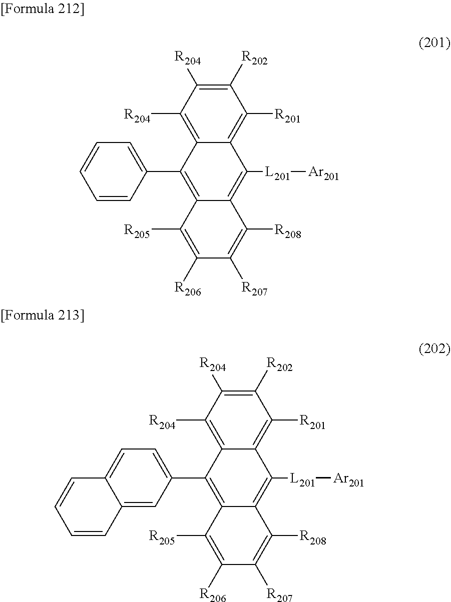

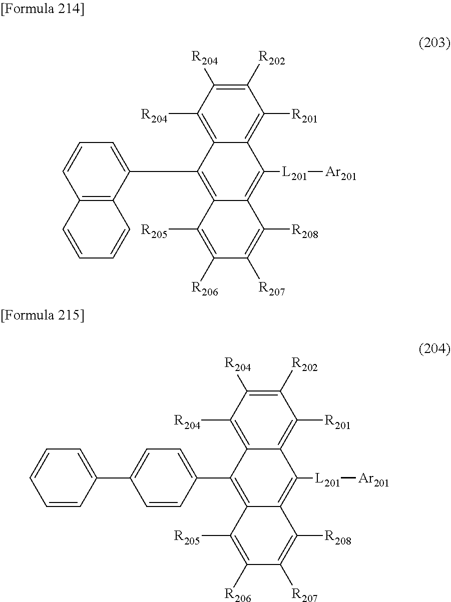

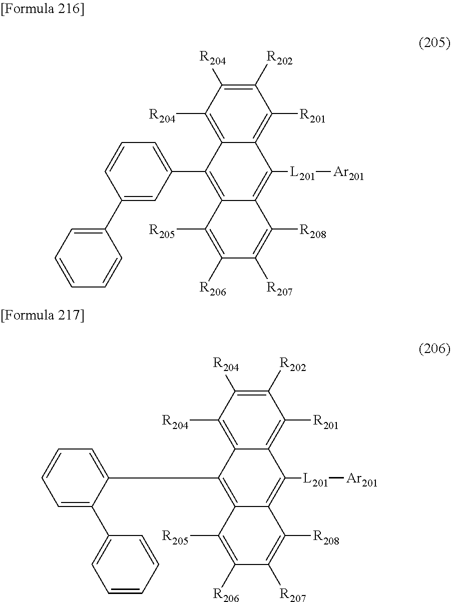

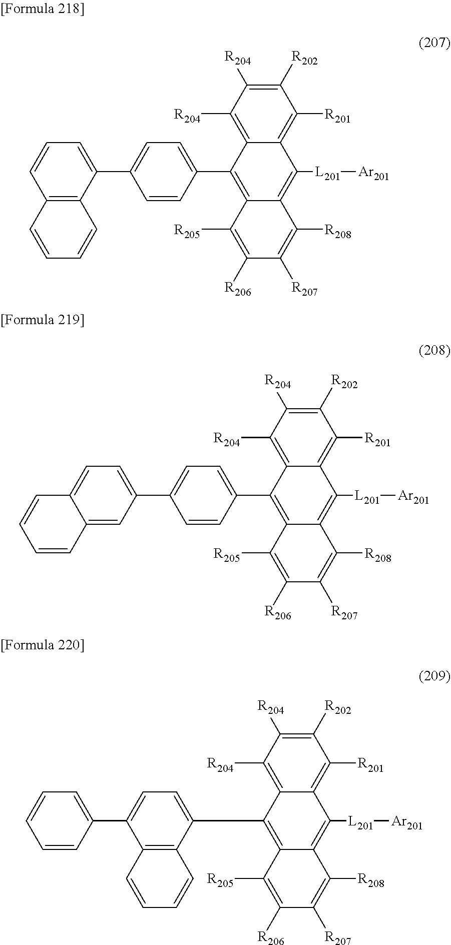

In the formulae (201) to (209), L₂₀₁ and Ar₂₀₁ respectively represent the same as L₂₀₁ and Ar₂₀₁ in the formula (H20); and R₂₀₁ to R₂₀₈ each independently represent the same as R₂₀₁ to R₂₀₈ in the formula (H20).

In an exemplary arrangement of the organic EL device according to the exemplary embodiment, the compound represented by the formula (H20) is a compound represented by a formula (221), a formula (222), a formula (223), a formula (224), a formula (225), a formula (226), a formula (227), a formula (228), or a formula (229) below.

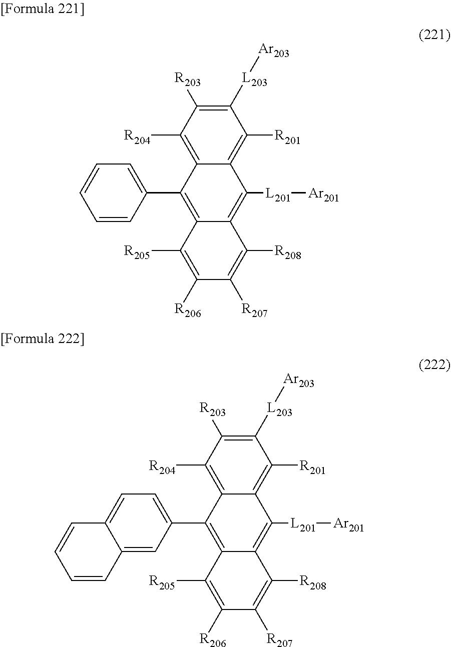

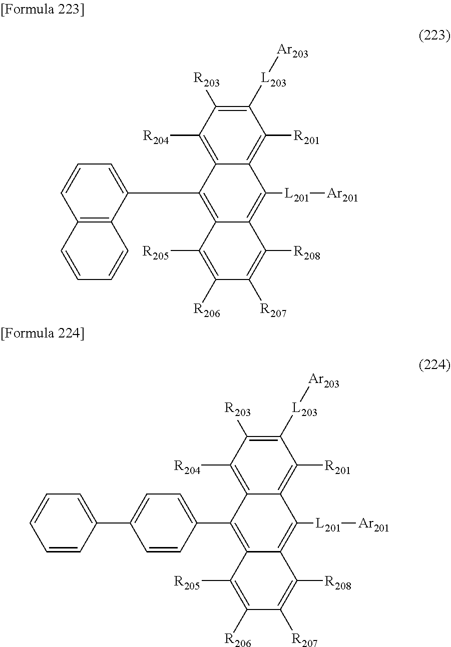

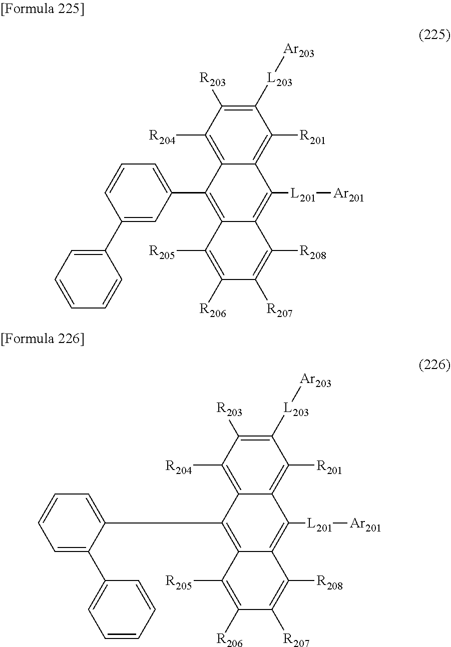

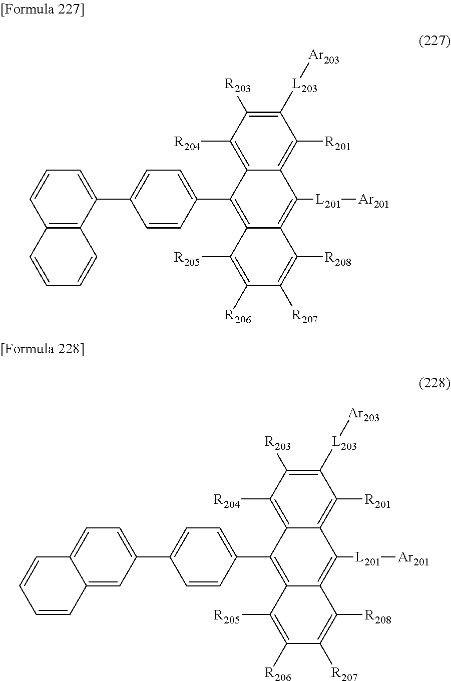

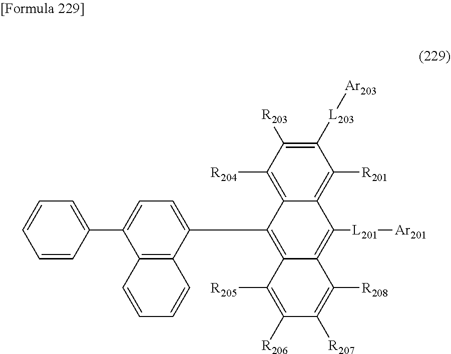

In the formulae (221), (222), (223), (224), (225), (226), (227), (228), and (229):
R₂₀₁ and R₂₀₃ to R₂₀₈ each independently represent the same as R₂₀₁ and R₂₀₃ to R₂₀₈ in the formula (H20);
L₂₀₁ and Ar₂₀₁ respectively represent the same as L₂₀₁ and Ar₂₀₁ in the formula (H20);
L₂₀₃ represents the same as L₂₀₃ in the formula (H21);
L₂₀₃ and L₂₀₁ are mutually the same or different;
Ar₂₀₃ represents the same as Ar₂₀₃ in the formula (H21); and
Ar₂₀₃ and Ar₂₀₁ are mutually the same or different.

In an exemplary arrangement of the organic EL device according to the exemplary embodiment, the compound represented by the formula (H20) is a compound represented by a formula (241), a formula (242), a formula (243), a formula (244), a formula (245), a formula (246), a formula (247), a formula (248), or a formula (249) below.

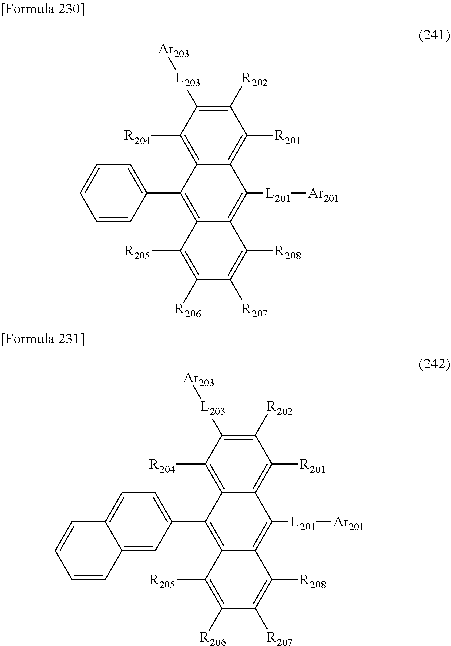

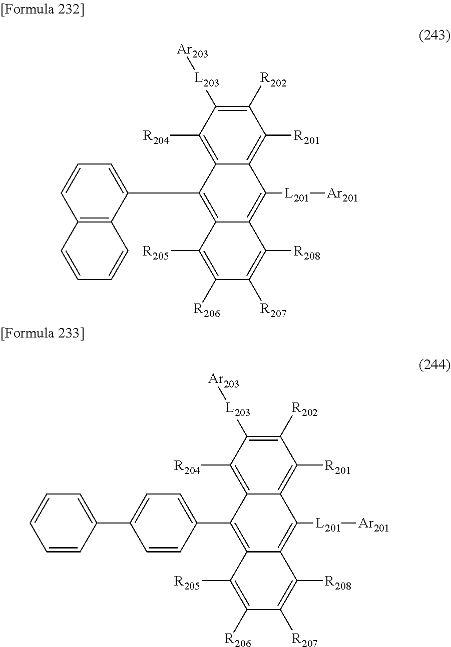

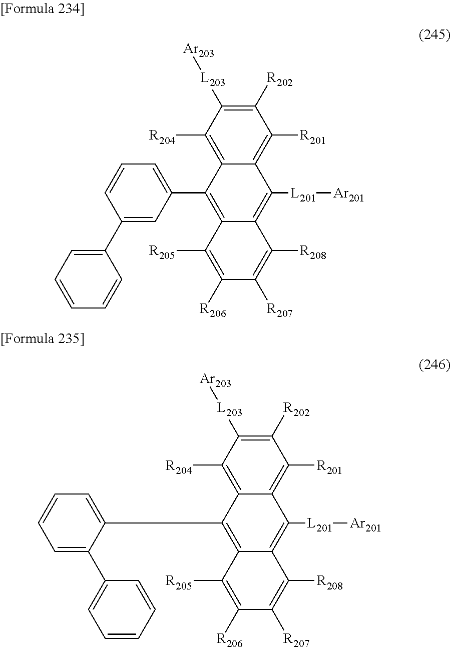

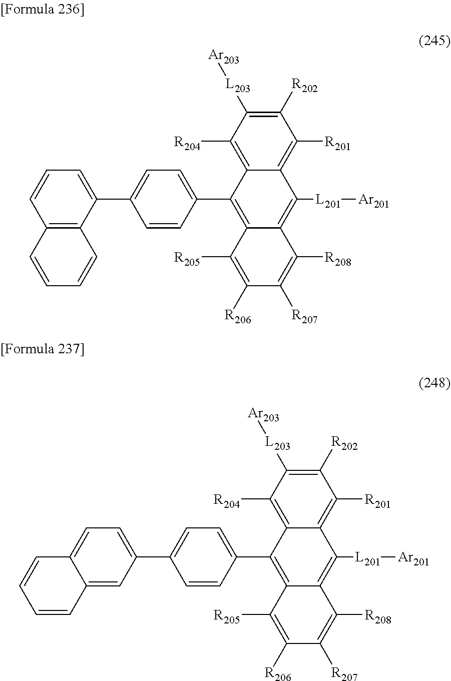

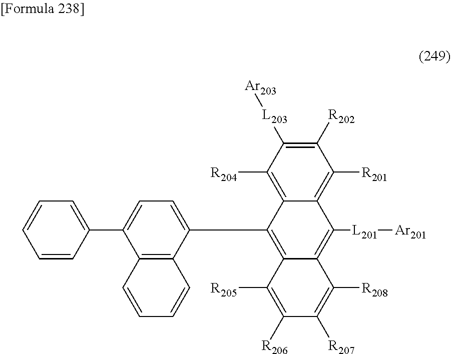

In the formulae (241), (242), (243), (244), (245), (246), (247), (248), and (249):
R₂₀₁, R₂₀₂ and R₂₀₄ to R₂₀ each independently represent the same as R₂₀₁, R₂₀₂ and R₂₀₄ to R₂₀ in the formula (H20);
L₂₀₁ and Ar₂₀₁ respectively represent the same as L₂₀₁ and Ar₂₀₁ in the formula (H20);
L₂₀₃ represents the same as L₂₀₃ in the formula (H21);
L₂₀₃ and L₂₀₁ are mutually the same or different;
Ar₂₀₃ represents the same as Ar₂₀₃ in the formula (H21); and
Ar₂₀₃ and Ar₂₀₁ are mutually the same or different.

In an exemplary arrangement of the organic EL device according to the exemplary embodiment, the first host material is represented by one of the formulae (201) to (209), the formulae (221) to (229), and the formulae (241) to (249).

In an exemplary arrangement of the organic EL device according to the exemplary embodiment, the first host material is represented by one of the formulae (201) to (209), the formulae (221) to (229), and the formulae (241) to (249), the first additional host material is represented by one of the formulae (201) to (209), the formulae (221) to (229), and the formulae (241) to (249), and the first host material and the first additional host material are mutually different compounds.

In this case, it is preferable that one of Ar₂₀₁ of the first host material and Ar₂₀₁ of the first additional host material is a substituted or unsubstituted aryl group having 6 to 50 ring carbon atoms and the other is a substituted or unsubstituted heterocyclic group having 5 to 50 ring atoms.

For instance, when the first host material is represented by the formula (203) and the first additional host material is represented by the formula (201), it is preferable that Ar₂₀₁ in the formula (203) is a substituted or unsubstituted aryl group having 6 to 50 ring carbon atoms and Ar₂₀₁ in the formula (201) is a substituted or unsubstituted heterocyclic group having 5 to 50 ring atoms.

Further, when the first host material is represented by the formula (201) and the first additional host material is represented by the formula (203), it is preferable that Ar₂₀₁ in the formula (201) is a substituted or unsubstituted aryl group having 6 to 50 ring carbon atoms and Ar₂₀₁ in the formula (203) is a substituted or unsubstituted heterocyclic group having 5 to 50 ring atoms.

In an arrangement of the organic EL device according to the exemplary embodiment, R₂₀₁ to R₂₀₈ in the formula (H20) are each independently a hydrogen atom, a substituted or unsubstituted alkyl group having 1 to 50 carbon atoms, a substituted or unsubstituted cycloalkyl group having 3 to 50 ring carbon atoms, or a group represented by —Si(R₉₀₁)(R₉₀₂)(R₉₀₃).

In an exemplary arrangement of the organic EL device according to the exemplary embodiment, in the formula (H20), L₂₀₁ is a single bond or an unsubstituted arylene group having 6 to 22 ring carbon atoms; and Ar₂₀₁ is a substituted or unsubstituted aryl group having 6 to 22 ring carbon atoms.

In an exemplary arrangement of the organic EL device according to the exemplary embodiment, R₂₀₁ to R₂₀₈ that are substituents of an anthracene skeleton in the formula (H20) are each preferably a hydrogen atom in terms of preventing inhibition of intermolecular interaction and inhibiting decrease in electron mobility. However, R₂₀₁ to R₂₀₈ may each be a substituted or unsubstituted aryl group having 6 to 50 ring carbon atoms or a substituted or unsubstituted heterocyclic group having 5 to 50 ring atoms.

In an exemplary arrangement of the organic EL device according to the exemplary embodiment, when the emitting region includes at least the first emitting layer containing the first host material represented by the formula (H10) and the first additional host material represented by the formula (H20) and the second emitting layer containing the second host material, R₃₀₁ to R₃₀₈ and R₂₀₁ to R₂₀₈ that are substituents of an anthracene skeleton in the compounds represented by the formulae (H10) and (H20) are each preferably a hydrogen atom in terms of preventing inhibition of intermolecular interaction and inhibiting decrease in electron mobility. However, R₃₀₁ to R₃₀₈ and R₂₀₁ to R₂₀₈ may each be a substituted or unsubstituted aryl group having 6 to 50 ring carbon atoms or a substituted or unsubstituted heterocyclic group having 5 to 50 ring atoms.

In an exemplary arrangement of the organic EL device according to the exemplary embodiment, the anode, the second emitting layer, the first emitting layer, and the cathode may be provided in this order. Alternatively, the anode, the first emitting layer, the second emitting layer, and the cathode may be provided in this order.

When the second emitting layer and the first emitting layer are layered in this order from a side close to the anode and the first host material contained in the first emitting layer is a compound represented by the formula (H10) and the first additional host material contained in the first emitting layer is a compound represented by the formula (H20), the following phenomenon may occur. It is thus preferable that R₃₀₁ to R₃₀₆ and R₂₀₁ to R₂₀₈ in the formulae (H10) and (H20) are each not a bulky substituent.

Assuming that R₃₀₁ to R₃₀₈ and R₂₀₁ to R₂₀₈ in the formulae (H10) and (H20) are each a bulky substituent such as an alkyl group and a cycloalkyl group, intermolecular interaction may be inhibited to decrease the electron mobility of the first host material and the first additional host material relative to that of the second host material, so that a relationship of μe(H1)>μe(H2) shown by a numerical formula (Numerical Formula 3) below may not be satisfied. When the first emitting layer contains the first host material represented by the formula (H10) and the first additional host material represented by the formula (H20), it can be expected that satisfying the relationship of μe(H1)>μe(H2) inhibits a decrease in a recombination ability between holes and electrons in the second emitting layer and a decrease in luminous efficiency. It should be noted that substituents, namely, a haloalkyl group, alkenyl group, alkynyl group, group represented by —Si(R₉₀₁)(R₉₀₂)(R₉₀₃), group represented by —O—(R₉₀₄), group represented by —S—(R₉₀₅), group represented by —N(R₉₀₆)(R₉₀₇), aralkyl group, group represented by —C(═O)R₈₀₁, group represented by —COOR₈₀₂, halogen atom, cyano group, and nitro group are likely to be bulky, and an alkyl group and cycloalkyl group are likely to be further bulky.

In the compounds represented by the formulae (H10) and (H20), R₃₀₁ to R₃₀₈ and R₂₀₁ to R₂₀₈, which are the substituents on the anthracene skeleton, are each preferably not a bulky substituent and preferably not an alkyl group and cycloalkyl group. More preferably, R₃₀₁ to R₃₀₈ and R₂₀₁ to R₂₀₈ are each not an alkyl group, cycloalkyl group, haloalkyl group, alkenyl group, alkynyl group, group represented by —Si(R₉₀₁)(R₉₀₂)(R₉₀₃), group represented by —O—(R₉₀₄), group represented by —S—(R₉₀₅), group represented by —N(R₉₀₆)(R₉₀₇), aralkyl group, group represented by —C(═O)R₈₀₁, group represented by —COOR₈₀₂, halogen atom, cyano group, and nitro group.

In the formulae (H10) and (H20), examples of the substituent for the “substituted or unsubstituted” group on R₃₀₁ to R₃₀₈ and R₂₀₁ to R₂₀₈ also preferably do not include the above-described substituent that is likely to be bulky, especially a substituted or unsubstituted alkyl group and a substituted or unsubstituted cycloalkyl group. When examples of the substituent for the “substituted or unsubstituted” group on R₃₀₁ to R₃₀₆ and R₂₀₁ to R₂₀₈ do not include a substituted or unsubstituted alkyl group and a substituted or unsubstituted cycloalkyl group, inhibition of intermolecular interaction to be caused by presence of a bulky substituent such as an alkyl group and a cycloalkyl group can be prevented, thereby preventing a decrease in the electron mobility. Moreover, when the compound described above is used as the first host material and the first additional host material in the first emitting layer, a decrease in a recombination ability between holes and electrons in the second emitting layer and a decrease in the luminous efficiency can be inhibited.

Further preferably, R₃₀₁ to R₃₀₈ and R₂₀₁ to R₂₀₈ that are substituents on the anthracene skeleton are not bulky substituents and R₃₀₁ to R₃₀₈ and R₂₀₁ to R₂₀₈ as substituents are unsubstituted. Assuming that R₃₀₁ to R₃₀₈ and R₂₀₁ to R₂₀₈ that are substituents on the anthracene skeleton are not bulky substituents and substituents are bonded to R₃₀₁ to R₃₀₈ and R₂₀₁ to R₂₀₈ that are not bulky substituents, the substituents bonded to R₃₀₁ to R₃₀₈ and R₂₀₁ to R₂₀₈ as substituents are preferably not bulky substituents; and the substituents bonded to R₃₀₁ to R₃₀₈ and R₂₀₁ to R₂₀₈ as substituents are preferably not an alkyl group and cycloalkyl group, more preferably not an alkyl group, cycloalkyl group, haloalkyl group, alkenyl group, alkynyl group, group represented by —Si(R₉₀₁)(R₉₀₂)(R₉₀₃), group represented by —O—(R₉₀₄), group represented by —S—(R₉₀₅), group represented by —N(R₉₀₆)(R₉₀₇), aralkyl group, group represented by —C(═O)R₈₀₁, group represented by —COOR₈₀₂, halogen atom, cyano group, and nitro group.

In the first host material, the groups specified to be “substituted or unsubstituted” are each preferably an “unsubstituted” group.

In the first additional host material, the groups specified to be “substituted or unsubstituted” are each preferably an “unsubstituted” group.

In an exemplary arrangement of the organic EL device according to the exemplary embodiment, the first host material substantially contains no deuterium atom and the first additional host material has at least one deuterium atom.

In an exemplary arrangement of the organic EL device according to the exemplary embodiment, the first emitting layer contains a compound represented by the formula (H10) as the first host material and a compound represented by the formula (H20) as the first additional host material, and at least one of the compound represented by the formula (H10) or the compound represented by the formula (H20) has at least one deuterium atom.

In an exemplary arrangement of the organic EL device according to the exemplary embodiment, the first emitting layer contains a compound represented by the formula (H10) as the first host material and a compound represented by the formula (H20) as the first additional host material, the compound represented by the formula (H10) substantially contains no deuterium atom, and the compound represented by the formula (H20) contains at least one deuterium atom.

In an exemplary arrangement of the organic EL device according to the exemplary embodiment, the first emitting layer contains a compound represented by the formula (H10) as the first host material and a compound represented by the formula (H20) as the first additional host material, the compound represented by the formula (H10) contains at least one deuterium atom, and the compound represented by the formula (H20) substantially contains no deuterium atom.

Here, the compound substantially contains no compound having a deuterium atom means that the compound contains no deuterium atom or that the compound is allowed to contain a deuterium atom approximately at the natural abundance ratio. The natural abundance ratio of deuterium atoms is, for example, 0.015% or less.

In an exemplary arrangement of the organic EL device according to the exemplary embodiment, the first emitting layer contains a compound represented by the formula (H10) as the first host material and a compound represented by the formula (H20) as the first additional host material, and each of the compound represented by the formula (H10) and the compound represented by the formula (H20) substantially contains no deuterium atom.

### Method for Producing First Host Material and First Additional Host Material

The first host material and the first additional host material can be produced by a known method. The first host material and the first additional host material can also be produced based on a known method through a known alternative reaction using a known material(s) tailored for the target compound.

### Specific Examples of First Host Material and First Additional Host Material

Specific examples of the first host material and the first additional host material include the following compounds. It should however be noted that the invention is not limited to the specific examples of the first host material and the first additional host material.

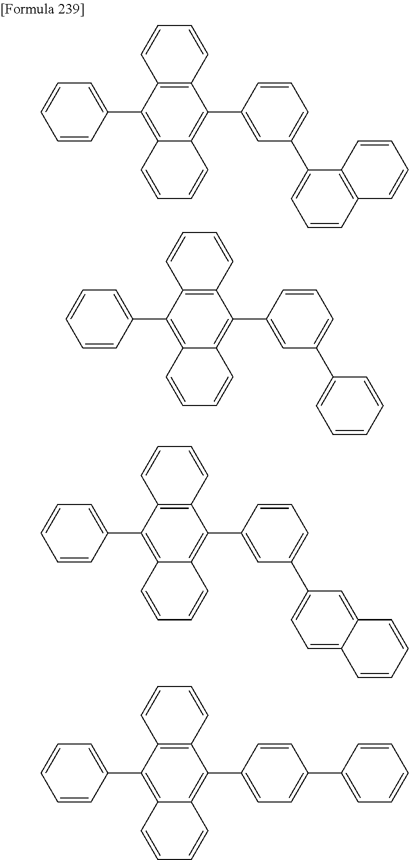

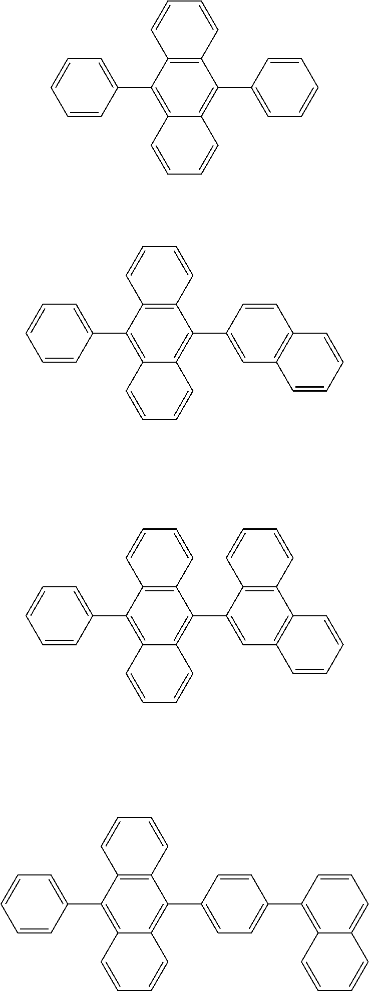

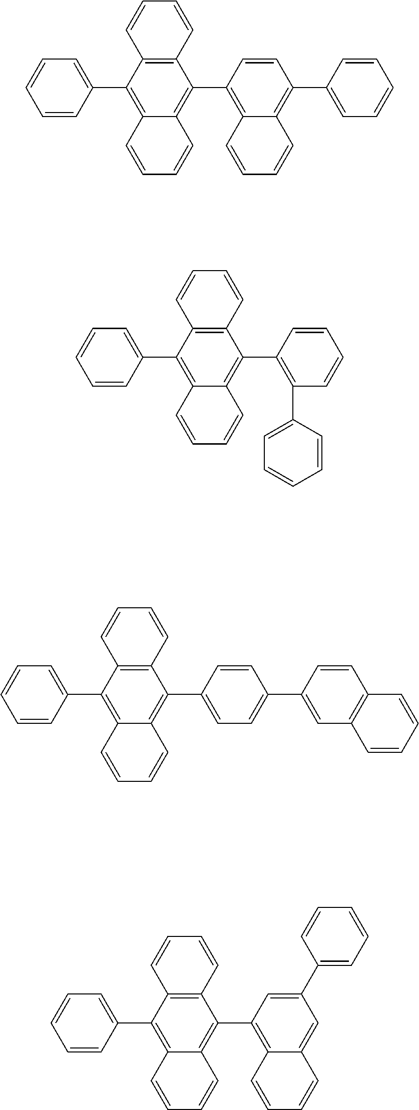

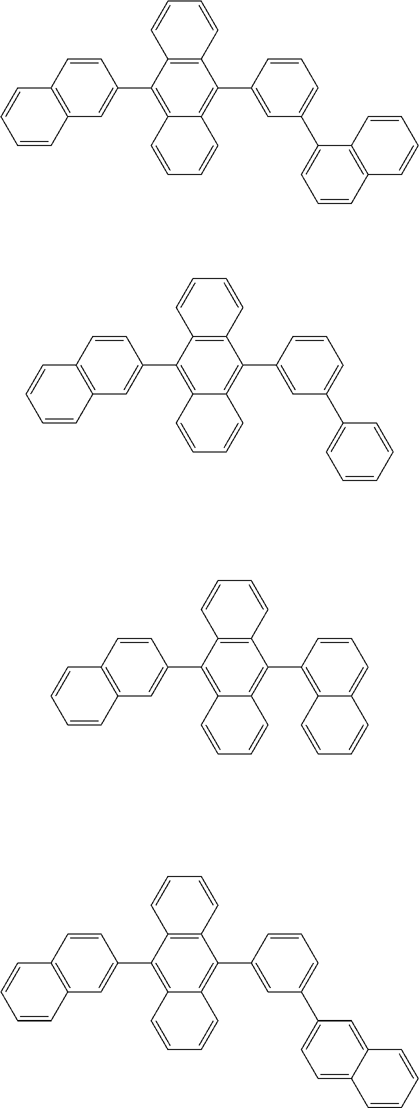

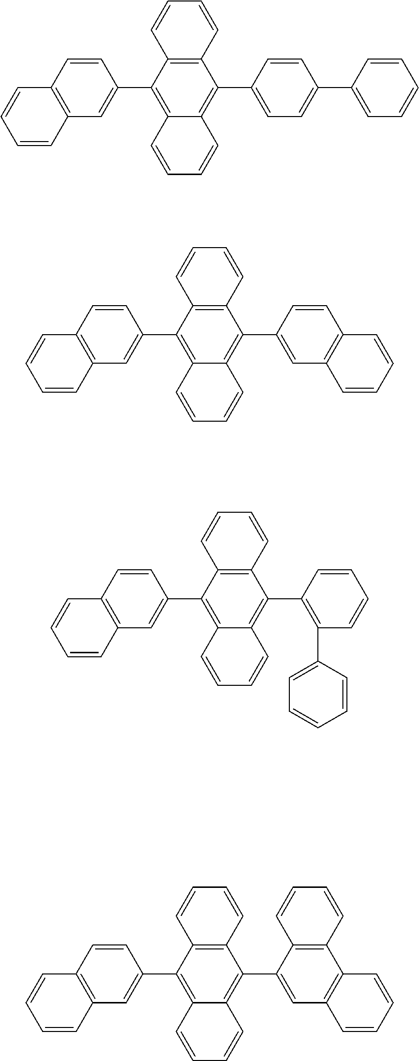

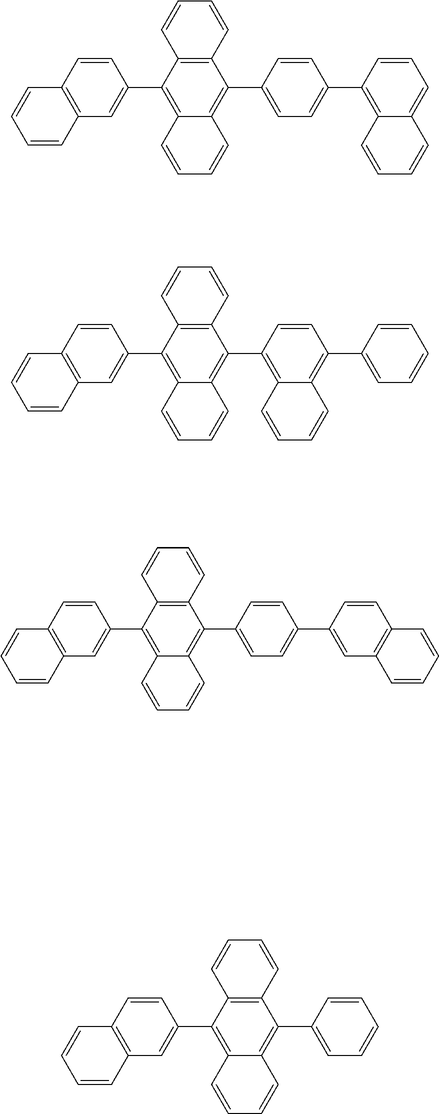

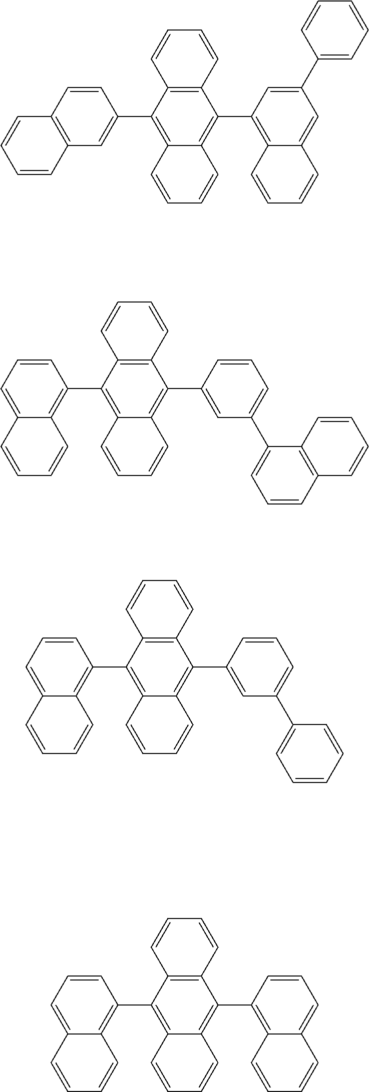

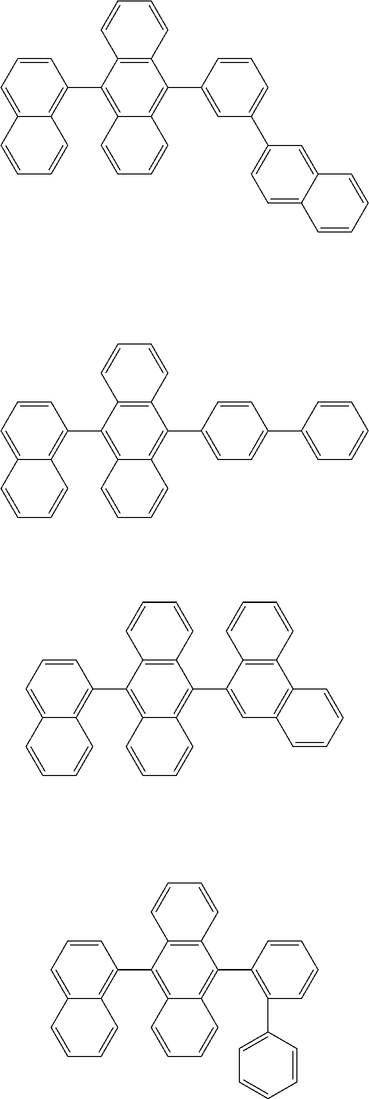

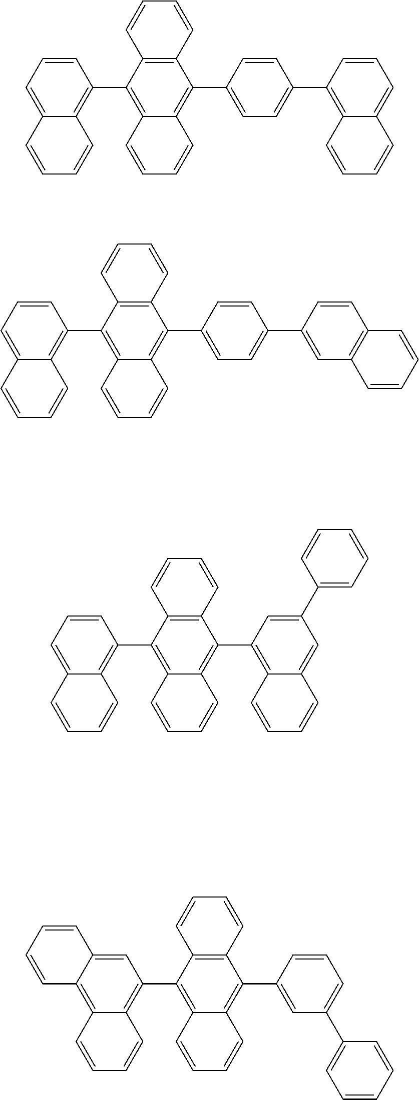

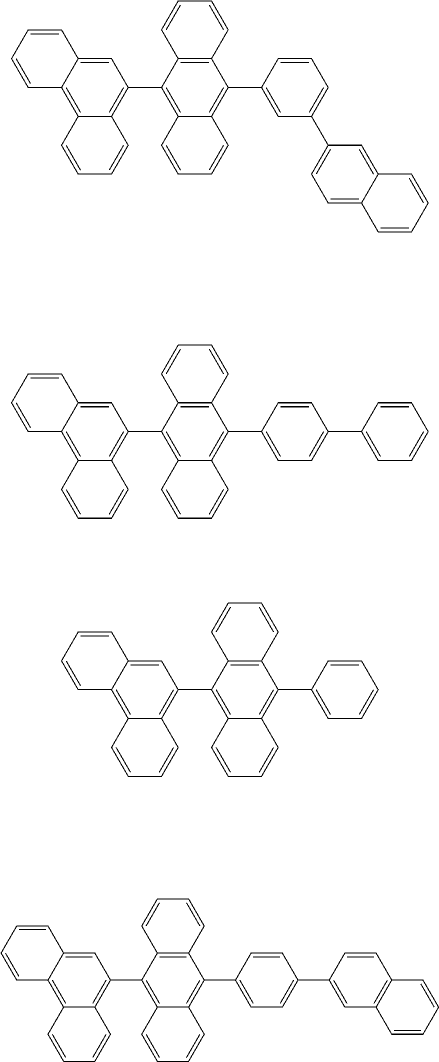

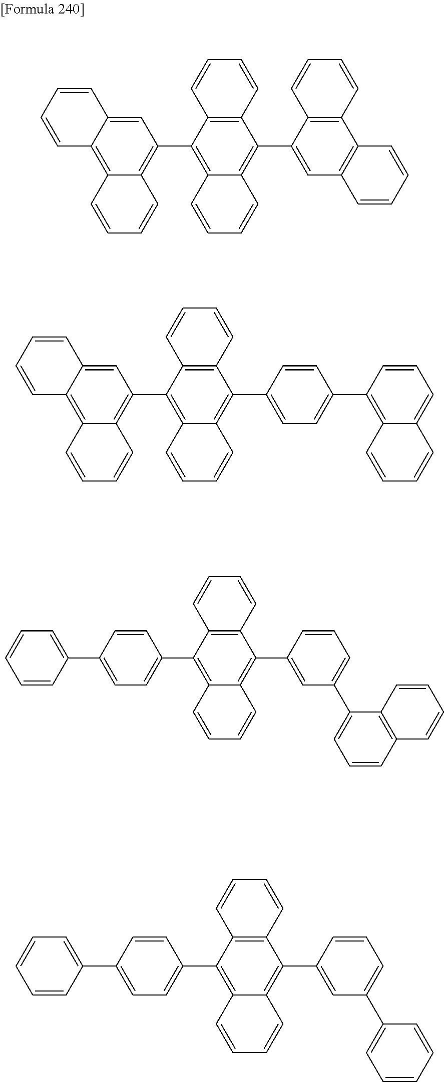

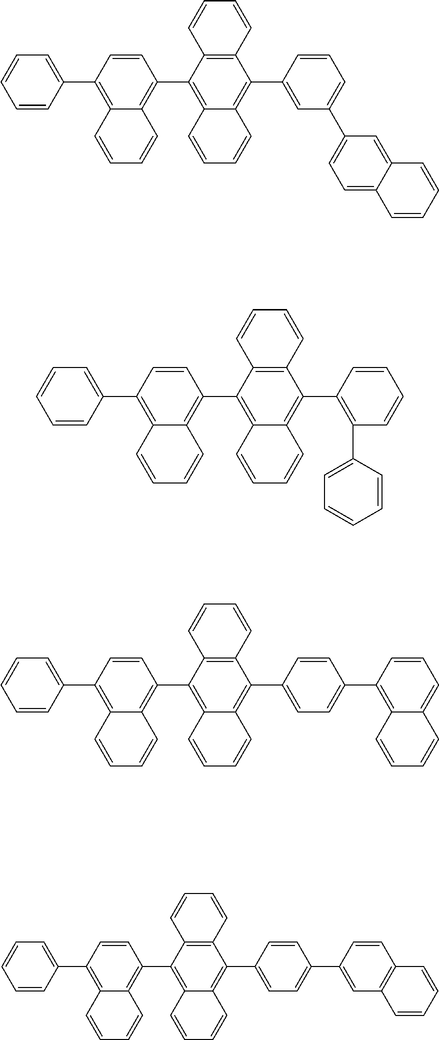

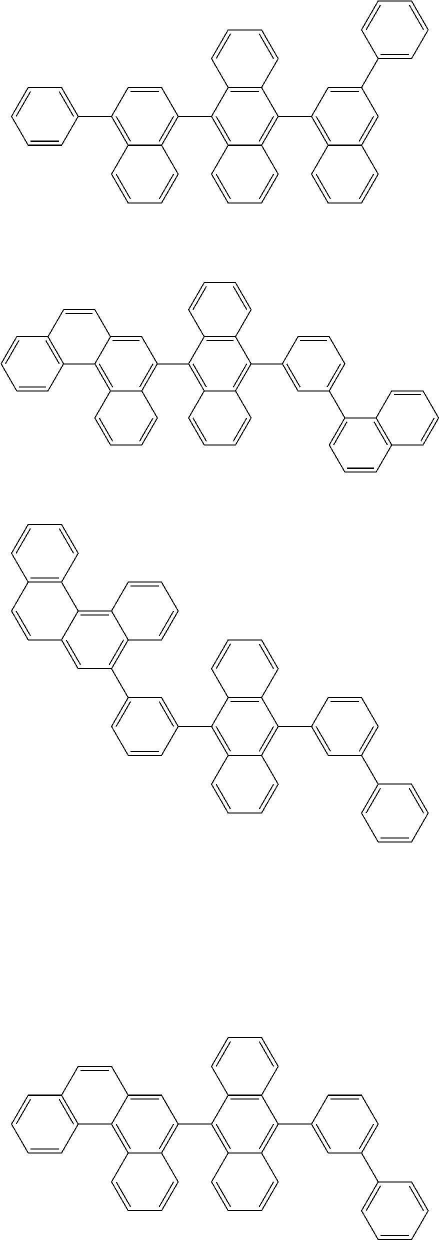

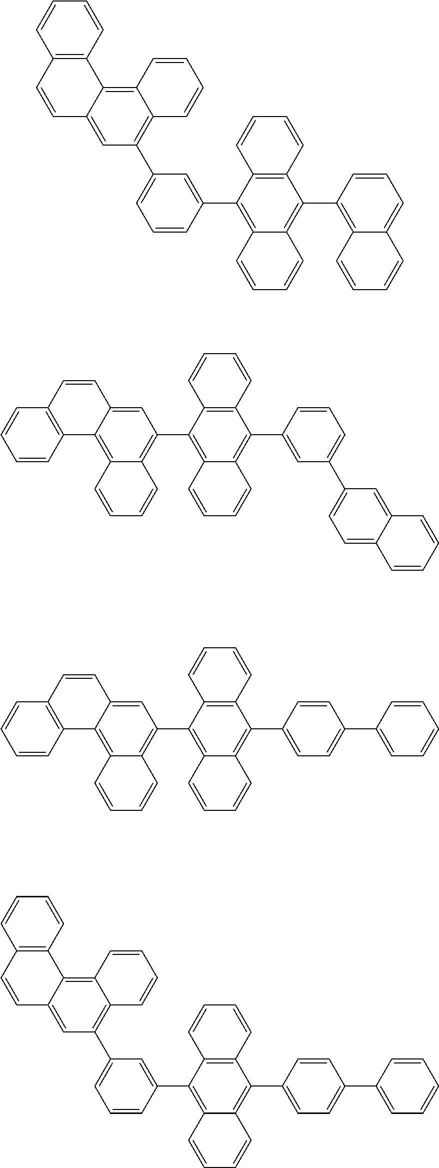

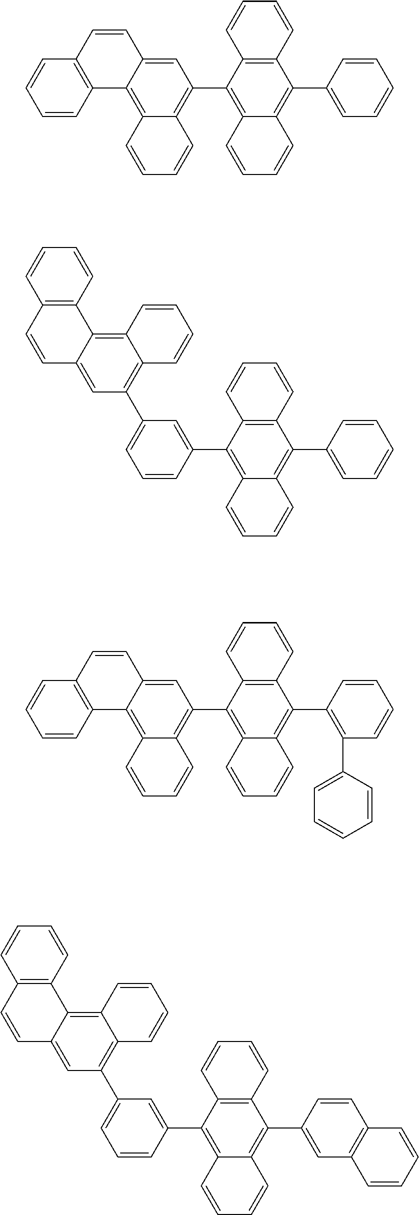

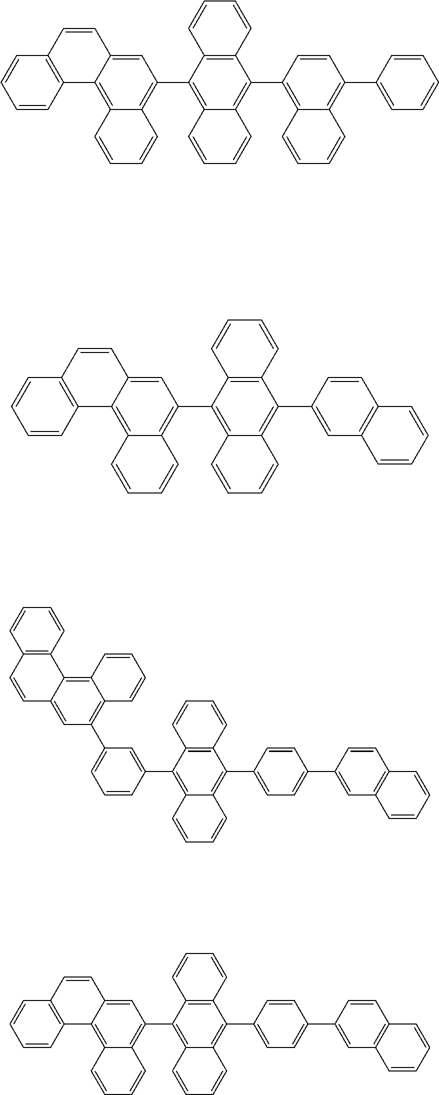

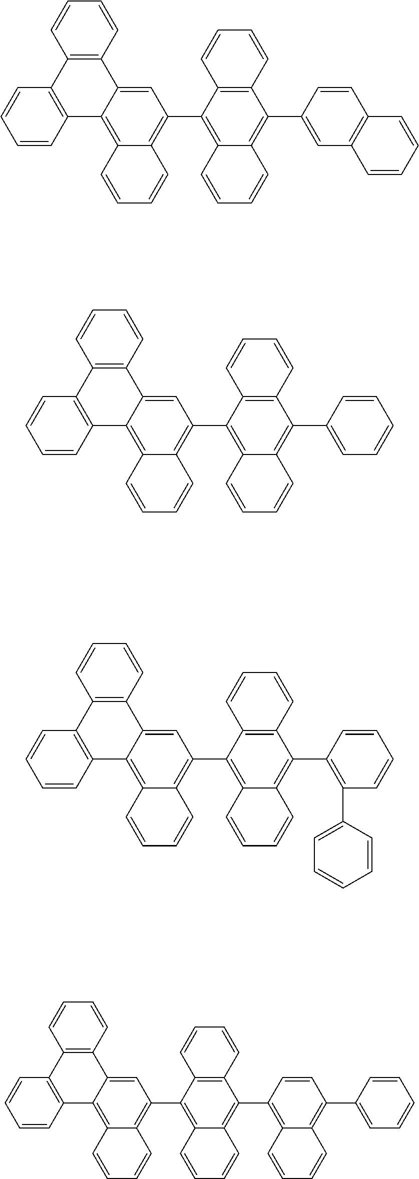

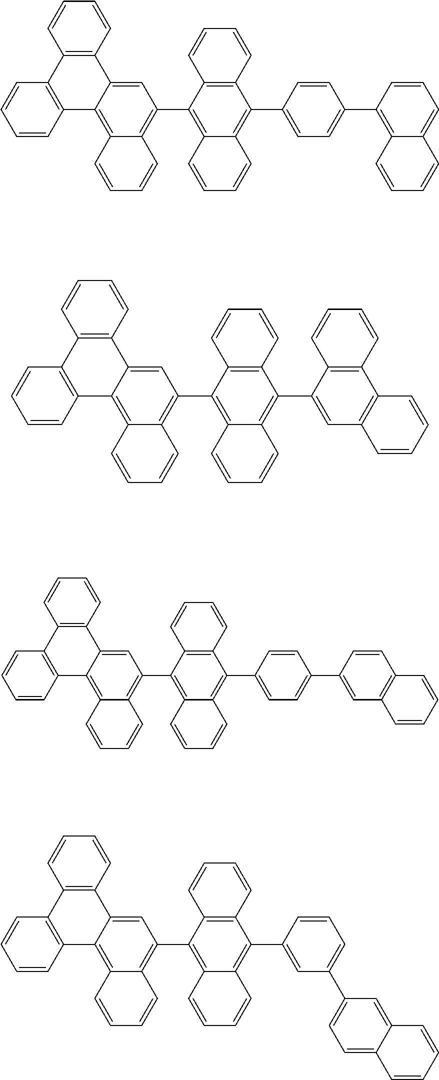

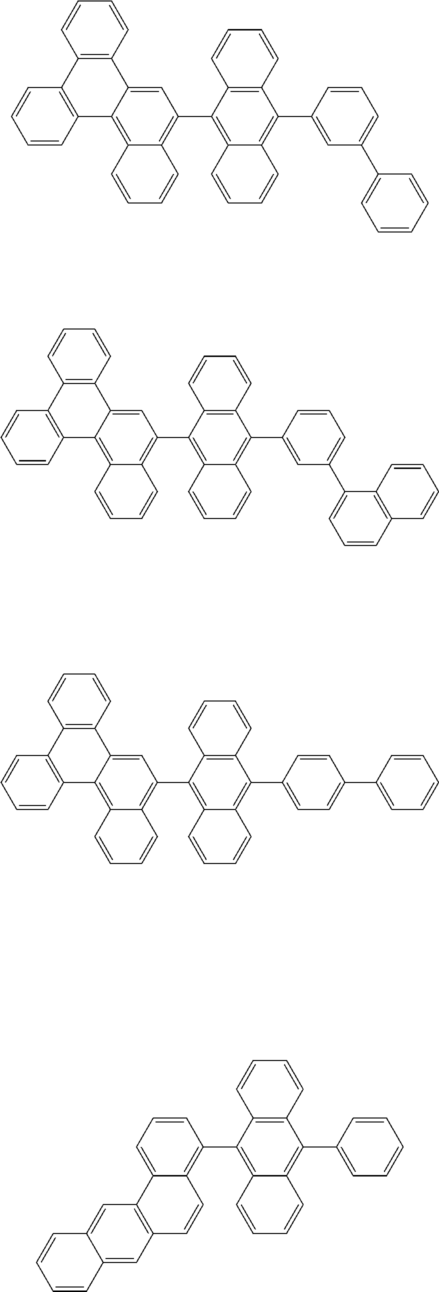

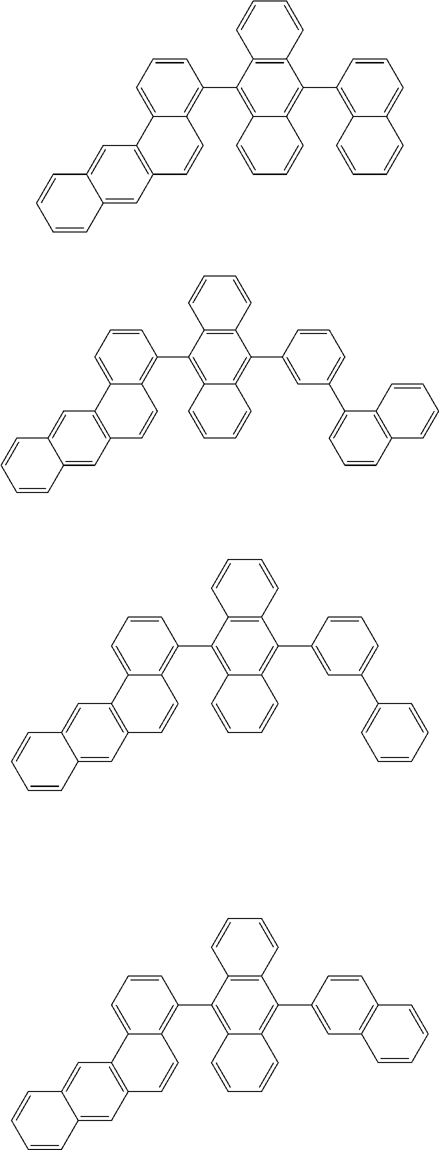

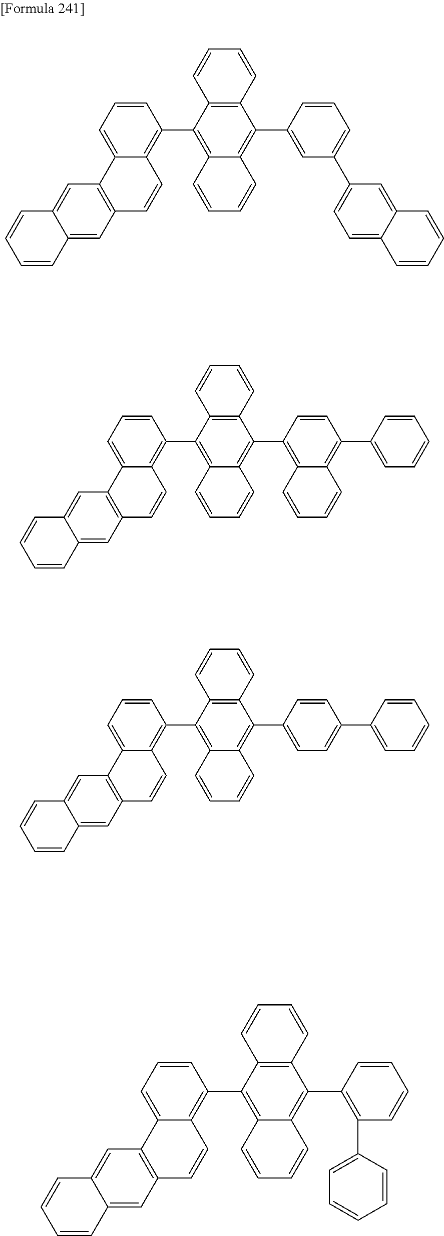

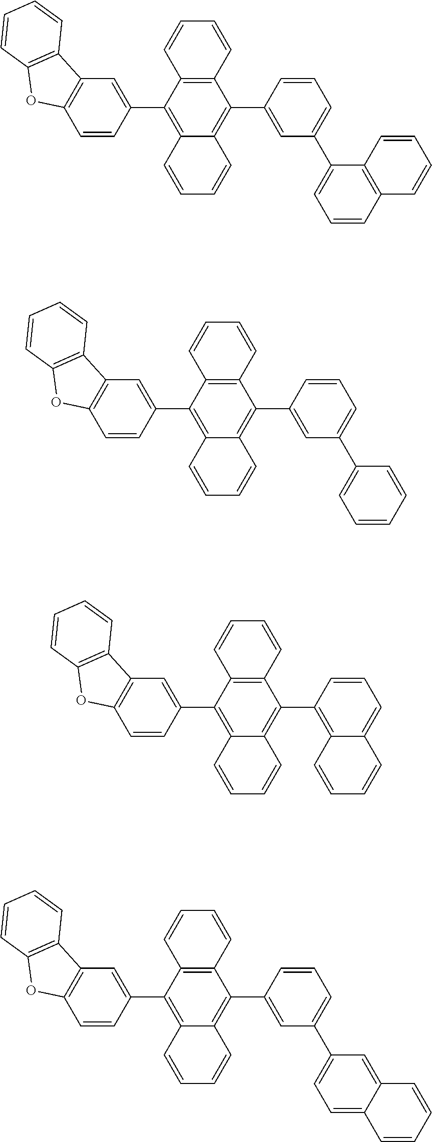 [SMILES: c1cc(-c2cccc3ccccc23)cc(-c2c3ccccc3c(-c3ccc4oc5ccccc5c4c3)c3ccccc23)c1 ; c1ccc(-c2cccc(-c3c4ccccc4c(-c4ccc5oc6ccccc6c5c4)c4ccccc34)c2)cc1 ; c1ccc2c(-c3c4ccccc4c(-c4ccc5oc6ccccc6c5c4)c4ccccc34)cccc2c1 ; c1cc(-c2ccc3ccccc3c2)cc(-c2c3ccccc3c(-c3ccc4oc5ccccc5c4c3)c3ccccc23)c1]

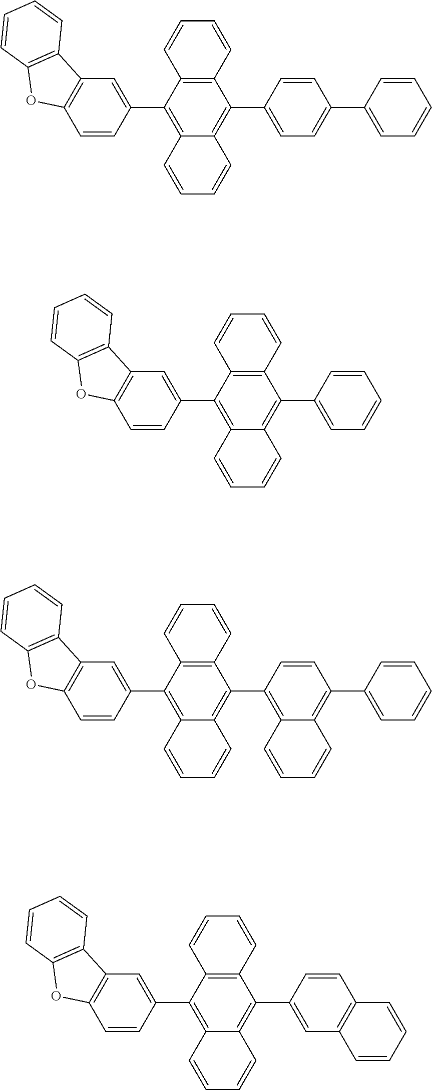 [SMILES: c1ccc(-c2ccc(-c3c4ccccc4c(-c4ccc5oc6ccccc6c5c4)c4ccccc34)cc2)cc1 ; c1ccc(-c2c3ccccc3c(-c3ccc4oc5ccccc5c4c3)c3ccccc23)cc1 ; c1ccc(-c2ccc(-c3c4ccccc4c(-c4ccc5oc6ccccc6c5c4)c4ccccc34)c3ccccc23)cc1 ; c1ccc2cc(-c3c4ccccc4c(-c4ccc5oc6ccccc6c5c4)c4ccccc34)ccc2c1]

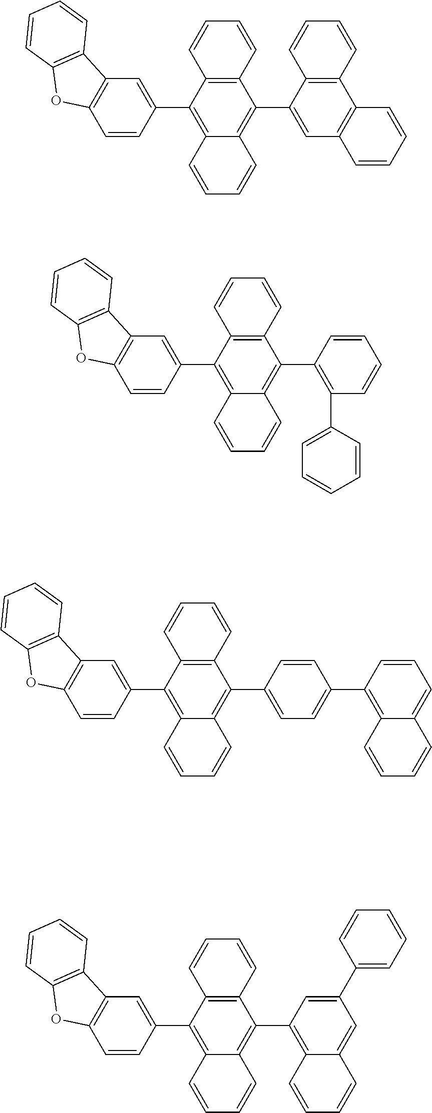 [SMILES: c1ccc2c(c1)cc(-c1c3ccccc3c(-c3ccc4oc5ccccc5c4c3)c3ccccc13)c1ccccc12 ; c1ccc(-c2ccccc2-c2c3ccccc3c(-c3ccc4oc5ccccc5c4c3)c3ccccc23)cc1 ; c1ccc2c(-c3ccc(-c4c5ccccc5c(-c5ccc6oc7ccccc7c6c5)c5ccccc45)cc3)cccc2c1 ; c1ccc(-c2cc(-c3c4ccccc4c(-c4ccc5oc6ccccc6c5c4)c4ccccc34)c3ccccc3c2)cc1]

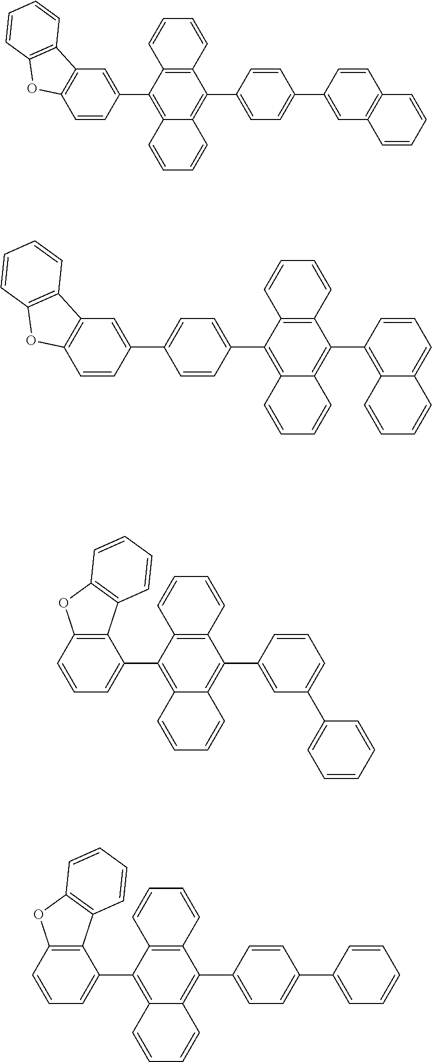 [SMILES: c1ccc2cc(-c3ccc(-c4c5ccccc5c(-c5ccc6oc7ccccc7c6c5)c5ccccc45)cc3)ccc2c1 ; c1ccc2c(-c3c4ccccc4c(-c4ccc(-c5ccc6oc7ccccc7c6c5)cc4)c4ccccc34)cccc2c1 ; c1ccc(-c2cccc(-c3c4ccccc4c(-c4cccc5oc6ccccc6c45)c4ccccc34)c2)cc1 ; c1ccc(-c2ccc(-c3c4ccccc4c(-c4cccc5oc6ccccc6c45)c4ccccc34)cc2)cc1]

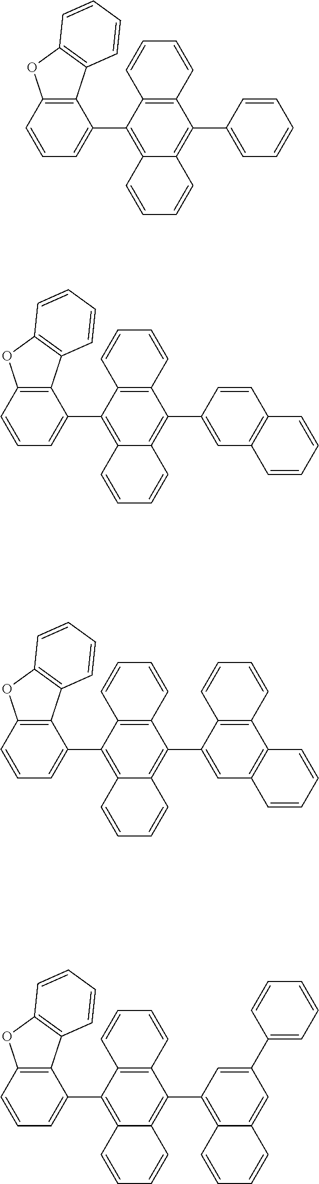 [SMILES: c1ccc(-c2c3ccccc3c(-c3cccc4oc5ccccc5c34)c3ccccc23)cc1 ; c1ccc2cc(-c3c4ccccc4c(-c4cccc5oc6ccccc6c45)c4ccccc34)ccc2c1 ; c1ccc2c(c1)cc(-c1c3ccccc3c(-c3cccc4oc5ccccc5c34)c3ccccc13)c1ccccc12 ; c1ccc(-c2cc(-c3c4ccccc4c(-c4cccc5oc6ccccc6c45)c4ccccc34)c3ccccc3c2)cc1]

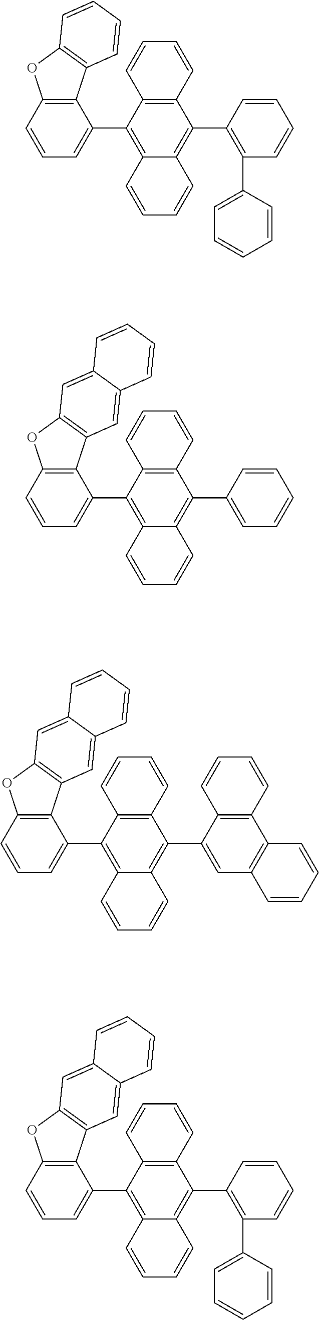 [SMILES: c1ccc(-c2ccccc2-c2c3ccccc3c(-c3cccc4oc5ccccc5c34)c3ccccc23)cc1 ; c1ccc(-c2c3ccccc3c(-c3cccc4oc5cc6ccccc6cc5c34)c3ccccc23)cc1 ; c1ccc2cc3c(cc2c1)oc1cccc(-c2c4ccccc4c(-c4cc5ccccc5c5ccccc45)c4ccccc24)c13 ; c1ccc(-c2ccccc2-c2c3ccccc3c(-c3cccc4oc5cc6ccccc6cc5c34)c3ccccc23)cc1]

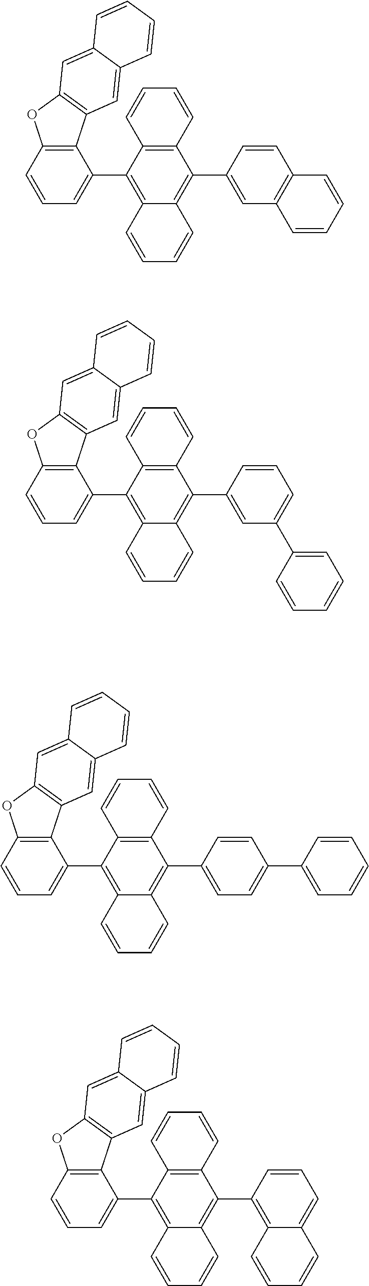 [SMILES: c1ccc2cc(-c3c4ccccc4c(-c4cccc5oc6cc7ccccc7cc6c45)c4ccccc34)ccc2c1 ; c1ccc(-c2cccc(-c3c4ccccc4c(-c4cccc5oc6cc7ccccc7cc6c45)c4ccccc34)c2)cc1 ; c1ccc(-c2ccc(-c3c4ccccc4c(-c4cccc5oc6cc7ccccc7cc6c45)c4ccccc34)cc2)cc1 ; c1ccc2cc3c(cc2c1)oc1cccc(-c2c4ccccc4c(-c4cccc5ccccc45)c4ccccc24)c13]

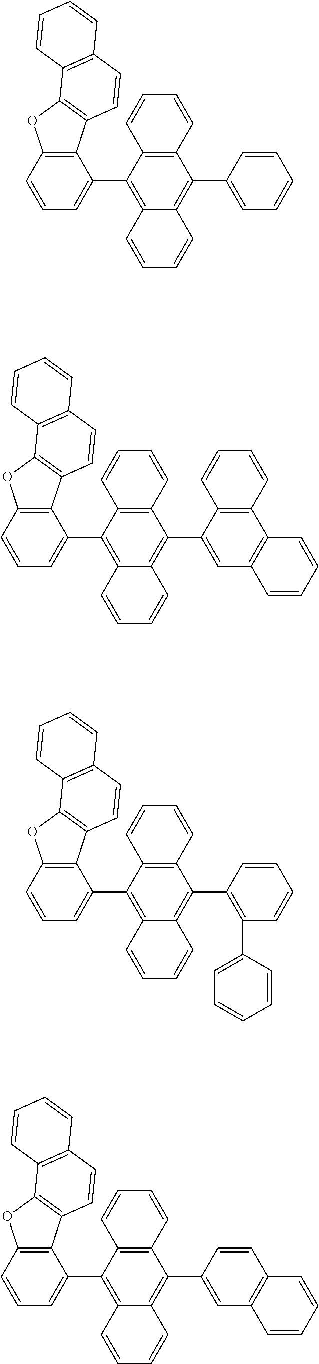 [SMILES: c1ccc(-c2c3ccccc3c(-c3cccc4oc5c6ccccc6ccc5c34)c3ccccc23)cc1 ; c1ccc2c(c1)cc(-c1c3ccccc3c(-c3cccc4oc5c6ccccc6ccc5c34)c3ccccc13)c1ccccc12 ; c1ccc(-c2ccccc2-c2c3ccccc3c(-c3cccc4oc5c6ccccc6ccc5c34)c3ccccc23)cc1 ; c1ccc2cc(-c3c4ccccc4c(-c4cccc5oc6c7ccccc7ccc6c45)c4ccccc34)ccc2c1]

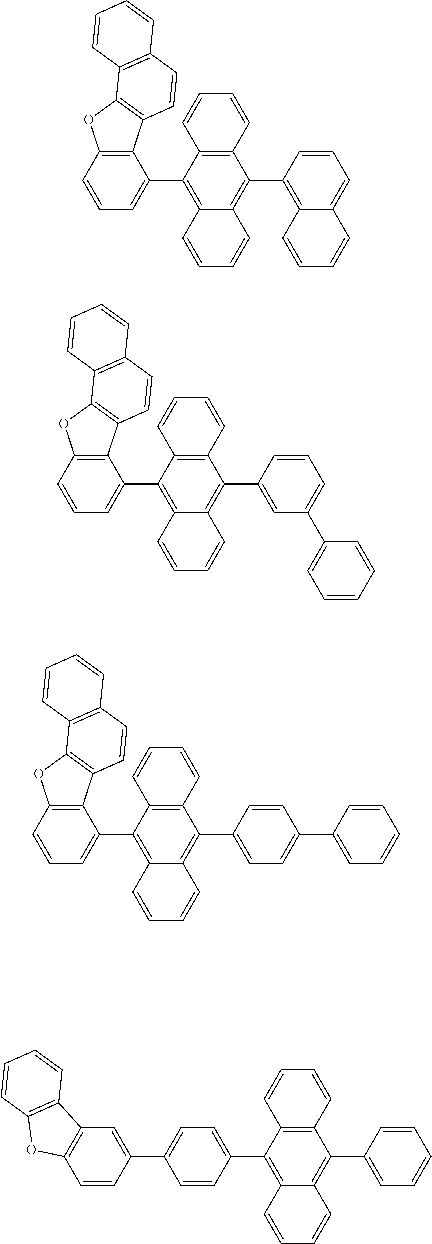 [SMILES: c1ccc2c(-c3c4ccccc4c(-c4cccc5oc6c7ccccc7ccc6c45)c4ccccc34)cccc2c1 ; c1ccc(-c2cccc(-c3c4ccccc4c(-c4cccc5oc6c7ccccc7ccc6c45)c4ccccc34)c2)cc1 ; c1ccc(-c2ccc(-c3c4ccccc4c(-c4cccc5oc6c7ccccc7ccc6c45)c4ccccc34)cc2)cc1 ; c1ccc(-c2c3ccccc3c(-c3ccc(-c4ccc5oc6ccccc6c5c4)cc3)c3ccccc23)cc1]

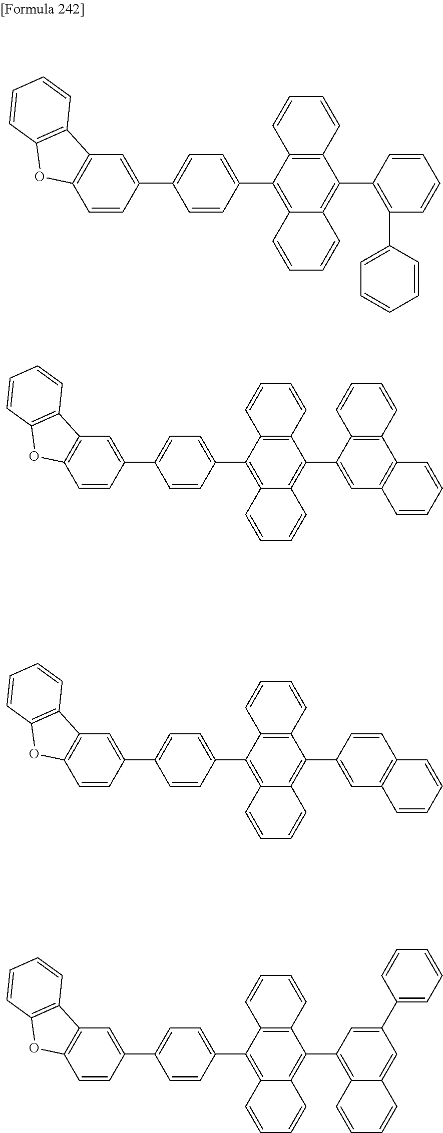 [SMILES: c1ccc(-c2ccccc2-c2c3ccccc3c(-c3ccc(-c4ccc5oc6ccccc6c5c4)cc3)c3ccccc23)cc1 ; c1ccc2c(c1)cc(-c1c3ccccc3c(-c3ccc(-c4ccc5oc6ccccc6c5c4)cc3)c3ccccc13)c1ccccc12 ; c1ccc2cc(-c3c4ccccc4c(-c4ccc(-c5ccc6oc7ccccc7c6c5)cc4)c4ccccc34)ccc2c1 ; c1ccc(-c2cc(-c3c4ccccc4c(-c4ccc(-c5ccc6oc7ccccc7c6c5)cc4)c4ccccc34)c3ccccc3c2)cc1]

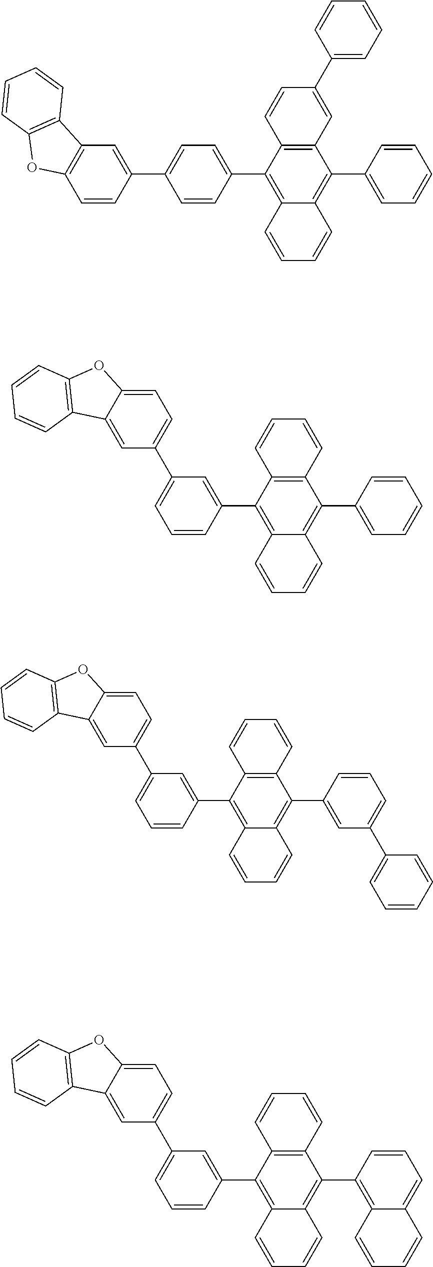 [SMILES: c1ccc(-c2ccc3c(-c4ccc(-c5ccc6oc7ccccc7c6c5)cc4)c4ccccc4c(-c4ccccc4)c3c2)cc1 ; c1ccc(-c2c3ccccc3c(-c3cccc(-c4ccc5oc6ccccc6c5c4)c3)c3ccccc23)cc1 ; c1ccc(-c2cccc(-c3c4ccccc4c(-c4cccc(-c5ccc6oc7ccccc7c6c5)c4)c4ccccc34)c2)cc1 ; c1cc(-c2ccc3oc4ccccc4c3c2)cc(-c2c3ccccc3c(-c3cccc4ccccc34)c3ccccc23)c1]

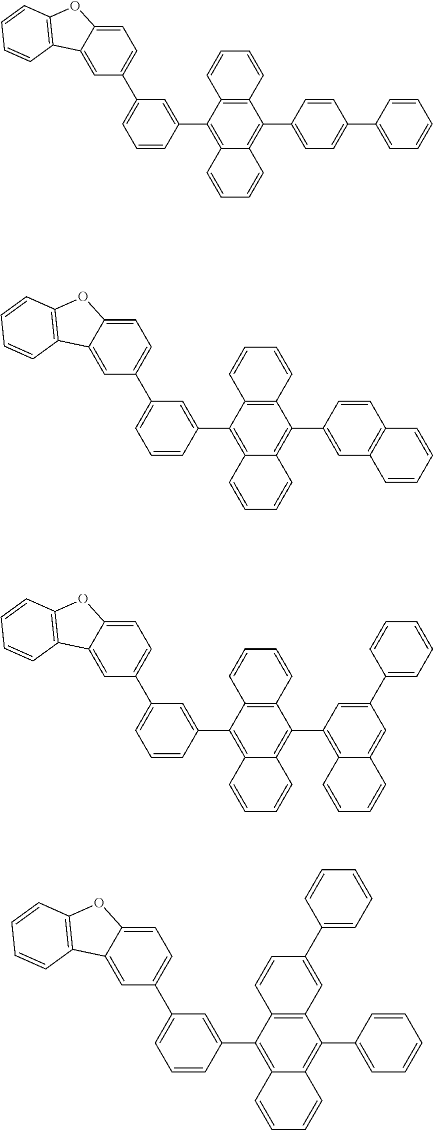 [SMILES: c1ccc(-c2ccc(-c3c4ccccc4c(-c4cccc(-c5ccc6oc7ccccc7c6c5)c4)c4ccccc34)cc2)cc1 ; c1cc(-c2ccc3oc4ccccc4c3c2)cc(-c2c3ccccc3c(-c3ccc4ccccc4c3)c3ccccc23)c1 ; c1ccc(-c2cc(-c3c4ccccc4c(-c4cccc(-c5ccc6oc7ccccc7c6c5)c4)c4ccccc34)c3ccccc3c2)cc1 ; c1ccc(-c2ccc3c(-c4cccc(-c5ccc6oc7ccccc7c6c5)c4)c4ccccc4c(-c4ccccc4)c3c2)cc1]

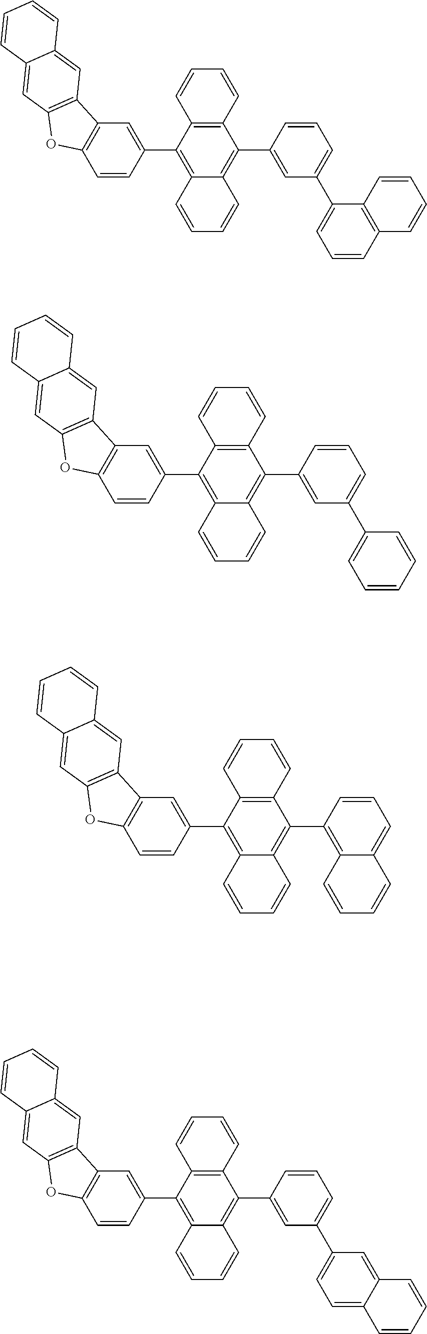 [SMILES: c1cc(-c2cccc3ccccc23)cc(-c2c3ccccc3c(-c3ccc4oc5cc6ccccc6cc5c4c3)c3ccccc23)c1 ; c1ccc(-c2cccc(-c3c4ccccc4c(-c4ccc5oc6cc7ccccc7cc6c5c4)c4ccccc34)c2)cc1 ; c1ccc2cc3c(cc2c1)oc1ccc(-c2c4ccccc4c(-c4cccc5ccccc45)c4ccccc24)cc13 ; c1cc(-c2ccc3ccccc3c2)cc(-c2c3ccccc3c(-c3ccc4oc5cc6ccccc6cc5c4c3)c3ccccc23)c1]

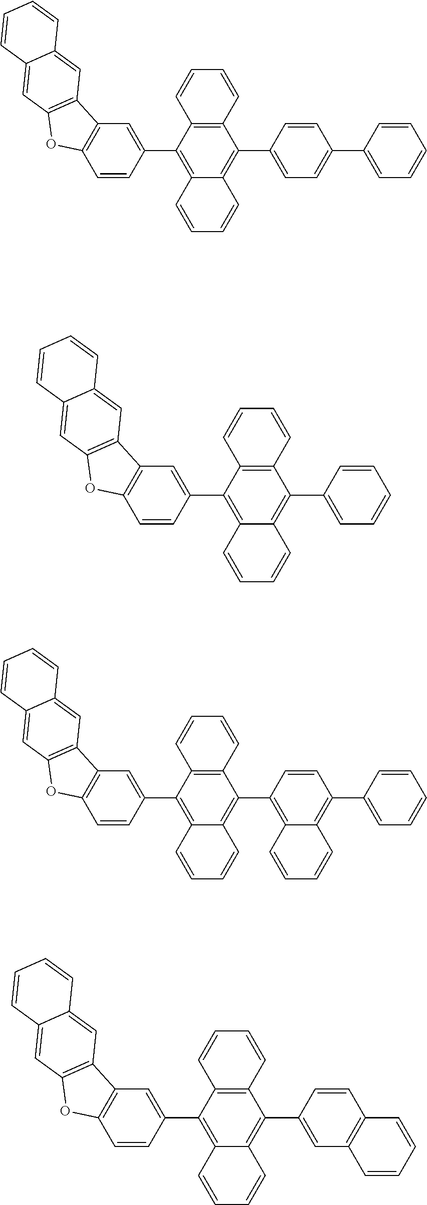 [SMILES: c1ccc(-c2ccc(-c3c4ccccc4c(-c4ccc5oc6cc7ccccc7cc6c5c4)c4ccccc34)cc2)cc1 ; c1ccc(-c2c3ccccc3c(-c3ccc4oc5cc6ccccc6cc5c4c3)c3ccccc23)cc1 ; c1ccc(-c2ccc(-c3c4ccccc4c(-c4ccc5oc6cc7ccccc7cc6c5c4)c4ccccc34)c3ccccc23)cc1 ; c1ccc2cc(-c3c4ccccc4c(-c4ccc5oc6cc7ccccc7cc6c5c4)c4ccccc34)ccc2c1]

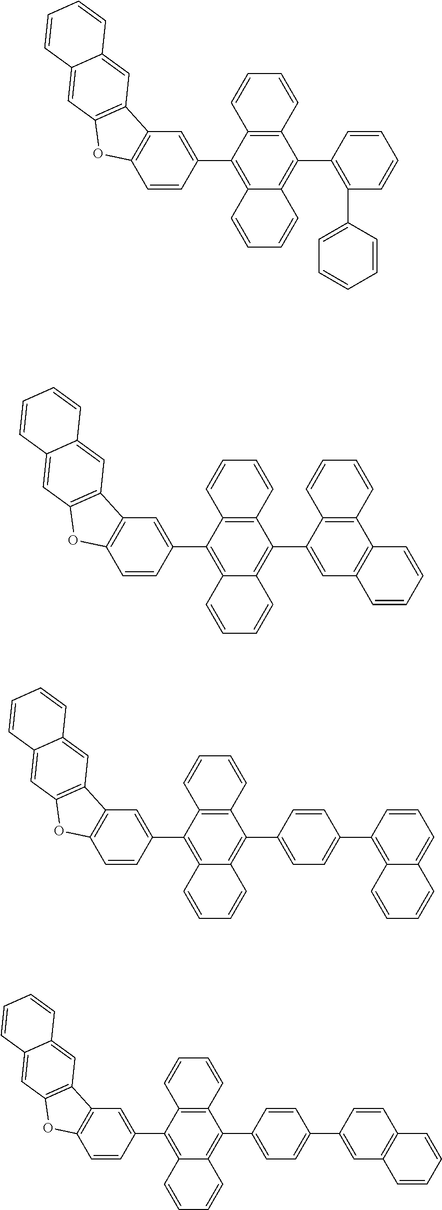 [SMILES: c1ccc(-c2ccccc2-c2c3ccccc3c(-c3ccc4oc5cc6ccccc6cc5c4c3)c3ccccc23)cc1 ; c1ccc2cc3c(cc2c1)oc1ccc(-c2c4ccccc4c(-c4cc5ccccc5c5ccccc45)c4ccccc24)cc13 ; c1ccc2cc3c(cc2c1)oc1ccc(-c2c4ccccc4c(-c4ccc(-c5cccc6ccccc56)cc4)c4ccccc24)cc13 ; c1ccc2cc(-c3ccc(-c4c5ccccc5c(-c5ccc6oc7cc8ccccc8cc7c6c5)c5ccccc45)cc3)ccc2c1]

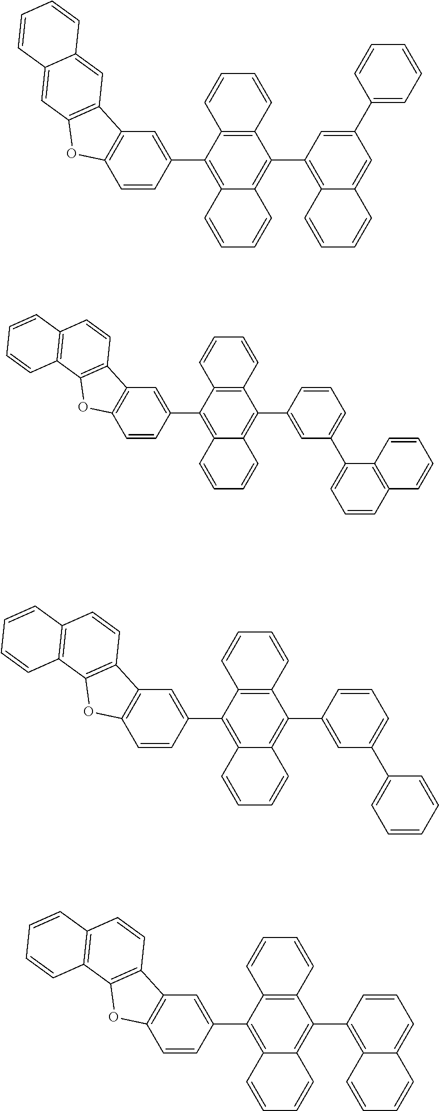 [SMILES: c1ccc(-c2cc(-c3c4ccccc4c(-c4ccc5oc6cc7ccccc7cc6c5c4)c4ccccc34)c3ccccc3c2)cc1 ; c1cc(-c2cccc3ccccc23)cc(-c2c3ccccc3c(-c3ccc4oc5c6ccccc6ccc5c4c3)c3ccccc23)c1 ; c1ccc(-c2cccc(-c3c4ccccc4c(-c4ccc5oc6c7ccccc7ccc6c5c4)c4ccccc34)c2)cc1 ; c1ccc2c(-c3c4ccccc4c(-c4ccc5oc6c7ccccc7ccc6c5c4)c4ccccc34)cccc2c1]

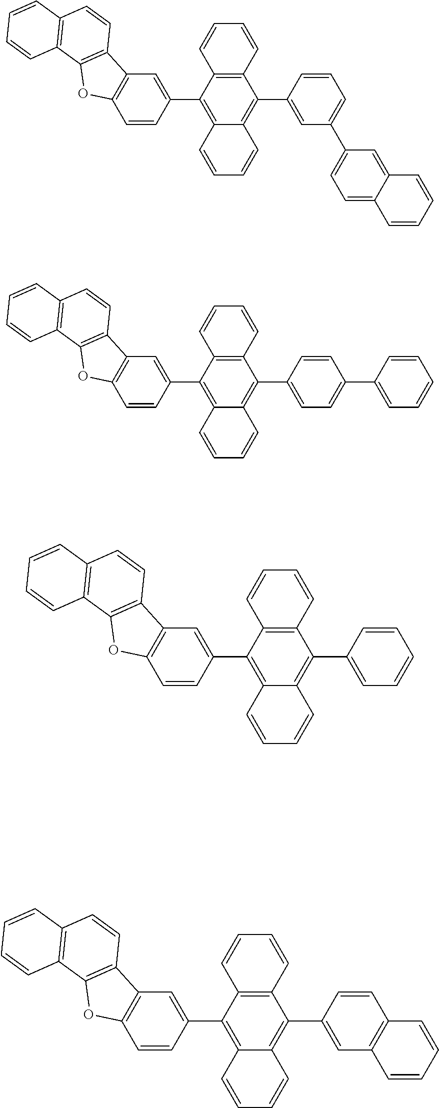 [SMILES: c1cc(-c2ccc3ccccc3c2)cc(-c2c3ccccc3c(-c3ccc4oc5c6ccccc6ccc5c4c3)c3ccccc23)c1 ; c1ccc(-c2ccc(-c3c4ccccc4c(-c4ccc5oc6c7ccccc7ccc6c5c4)c4ccccc34)cc2)cc1 ; c1ccc(-c2c3ccccc3c(-c3ccc4oc5c6ccccc6ccc5c4c3)c3ccccc23)cc1 ; c1ccc2cc(-c3c4ccccc4c(-c4ccc5oc6c7ccccc7ccc6c5c4)c4ccccc34)ccc2c1]

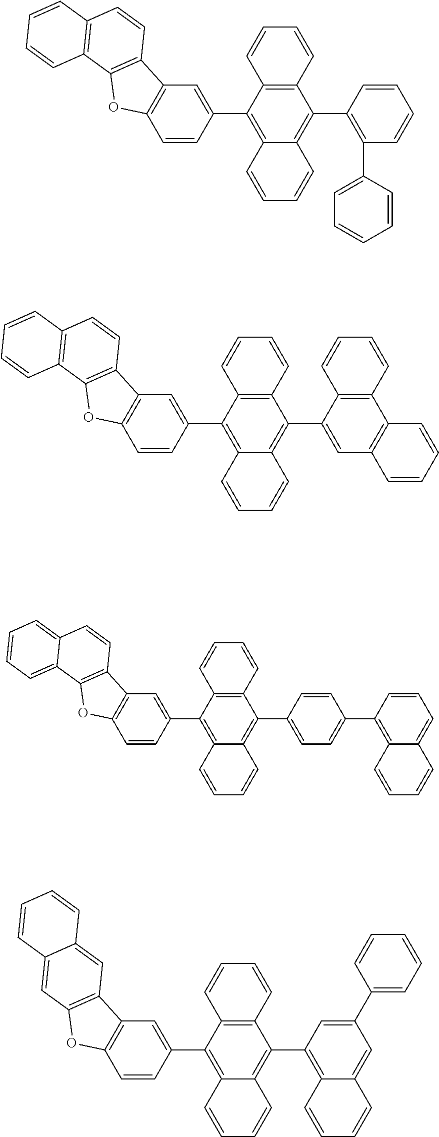 [SMILES: c1ccc(-c2ccccc2-c2c3ccccc3c(-c3ccc4oc5c6ccccc6ccc5c4c3)c3ccccc23)cc1 ; c1ccc2c(c1)cc(-c1c3ccccc3c(-c3ccc4oc5c6ccccc6ccc5c4c3)c3ccccc13)c1ccccc12 ; c1ccc2c(-c3ccc(-c4c5ccccc5c(-c5ccc6oc7c8ccccc8ccc7c6c5)c5ccccc45)cc3)cccc2c1 ; c1ccc(-c2cc(-c3c4ccccc4c(-c4ccc5oc6cc7ccccc7cc6c5c4)c4ccccc34)c3ccccc3c2)cc1]

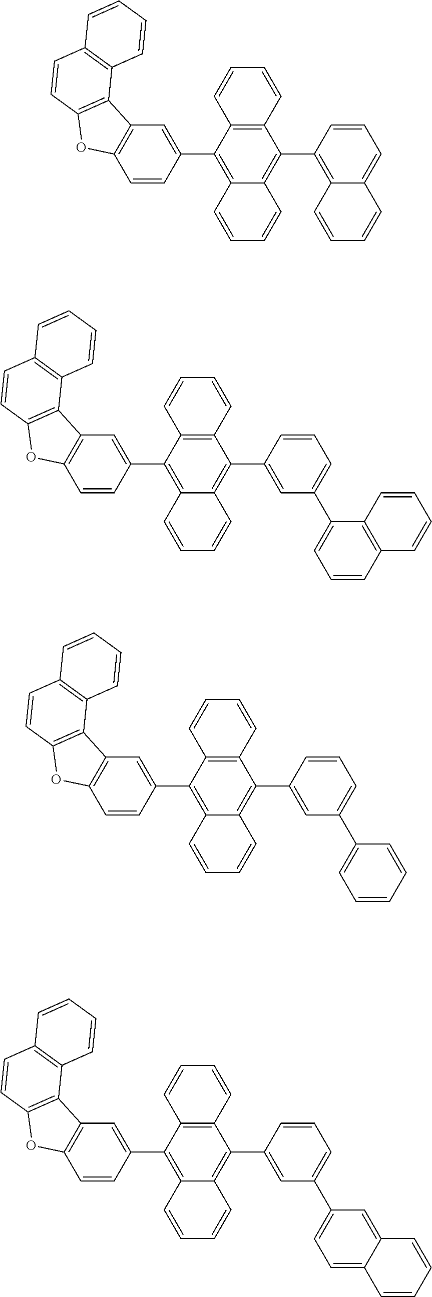 [SMILES: c1ccc2c(-c3c4ccccc4c(-c4ccc5oc6ccc7ccccc7c6c5c4)c4ccccc34)cccc2c1 ; c1cc(-c2cccc3ccccc23)cc(-c2c3ccccc3c(-c3ccc4oc5ccc6ccccc6c5c4c3)c3ccccc23)c1 ; c1ccc(-c2cccc(-c3c4ccccc4c(-c4ccc5oc6ccc7ccccc7c6c5c4)c4ccccc34)c2)cc1 ; c1cc(-c2ccc3ccccc3c2)cc(-c2c3ccccc3c(-c3ccc4oc5ccc6ccccc6c5c4c3)c3ccccc23)c1]

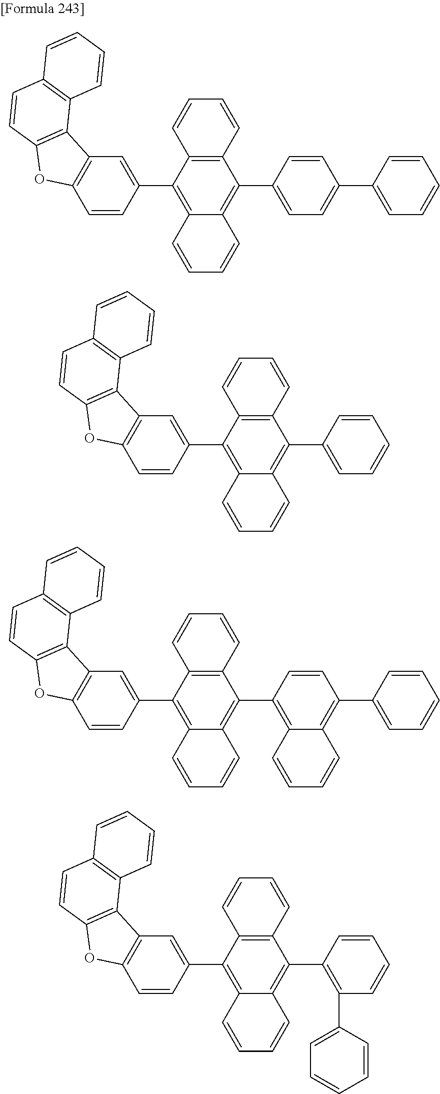 [SMILES: c1ccc(-c2ccc(-c3c4ccccc4c(-c4ccc5oc6ccc7ccccc7c6c5c4)c4ccccc34)cc2)cc1 ; c1ccc(-c2c3ccccc3c(-c3ccc4oc5ccc6ccccc6c5c4c3)c3ccccc23)cc1 ; c1ccc(-c2ccc(-c3c4ccccc4c(-c4ccc5oc6ccc7ccccc7c6c5c4)c4ccccc34)c3ccccc23)cc1 ; c1ccc(-c2ccccc2-c2c3ccccc3c(-c3ccc4oc5ccc6ccccc6c5c4c3)c3ccccc23)cc1]

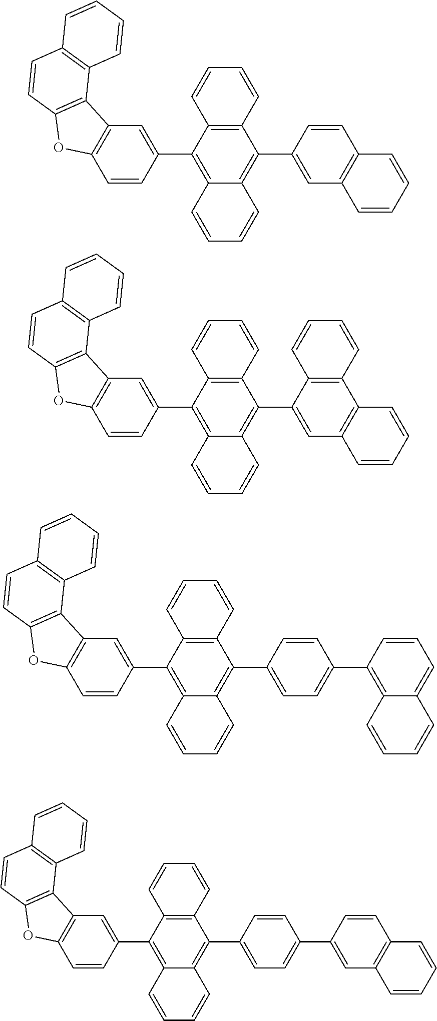 [SMILES: c1ccc2cc(-c3c4ccccc4c(-c4ccc5oc6ccc7ccccc7c6c5c4)c4ccccc34)ccc2c1 ; c1ccc2c(c1)cc(-c1c3ccccc3c(-c3ccc4oc5ccc6ccccc6c5c4c3)c3ccccc13)c1ccccc12 ; c1ccc2c(-c3ccc(-c4c5ccccc5c(-c5ccc6oc7ccc8ccccc8c7c6c5)c5ccccc45)cc3)cccc2c1 ; c1ccc2cc(-c3ccc(-c4c5ccccc5c(-c5ccc6oc7ccc8ccccc8c7c6c5)c5ccccc45)cc3)ccc2c1]

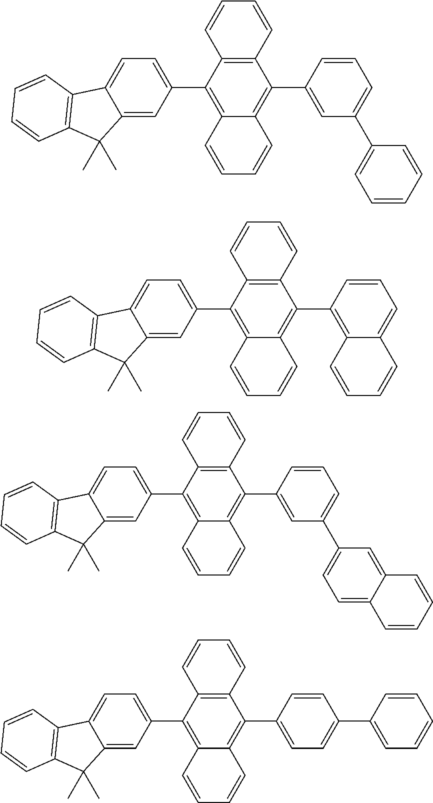

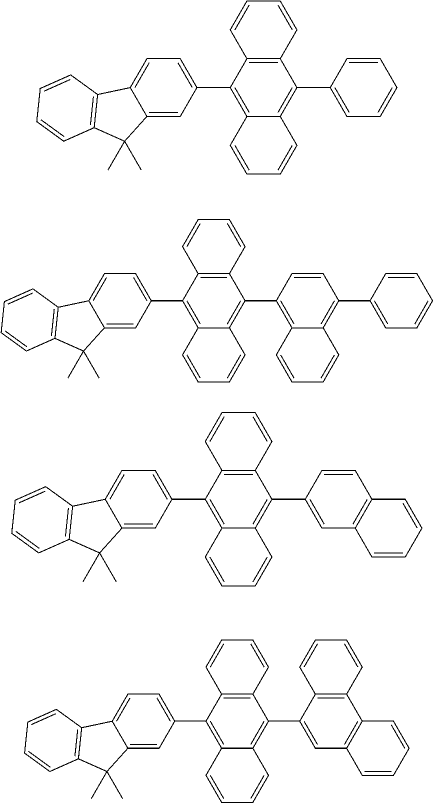

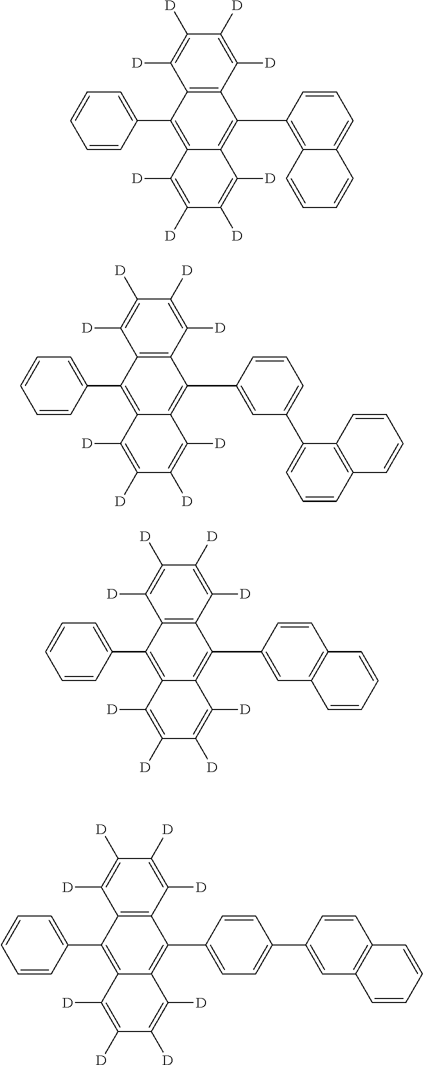

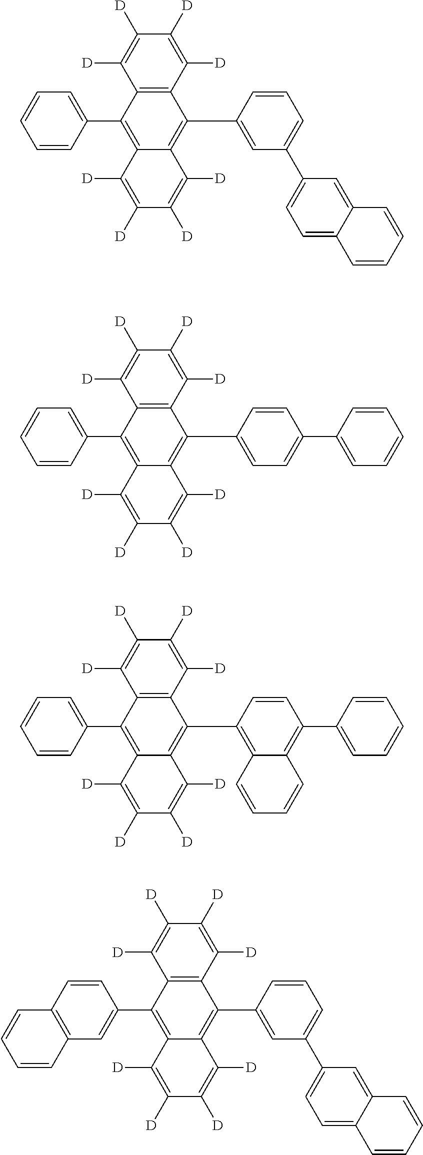 [SMILES: [2H]c1c([2H])c([2H])c2c(-c3cccc(-c4ccc5ccccc5c4)c3)c3c([2H])c([2H])c([2H])c([2H])c3c(-c3ccccc3)c2c1[2H] ; [2H]c1c([2H])c([2H])c2c(-c3ccc(-c4ccccc4)cc3)c3c([2H])c([2H])c([2H])c([2H])c3c(-c3ccccc3)c2c1[2H] ; [2H]c1c([2H])c([2H])c2c(-c3ccc(-c4ccccc4)c4ccccc34)c3c([2H])c([2H])c([2H])c([2H])c3c(-c3ccccc3)c2c1[2H] ; [2H]c1c([2H])c([2H])c2c(-c3ccc4ccccc4c3)c3c([2H])c([2H])c([2H])c([2H])c3c(-c3cccc(-c4ccc5ccccc5c4)c3)c2c1[2H]]

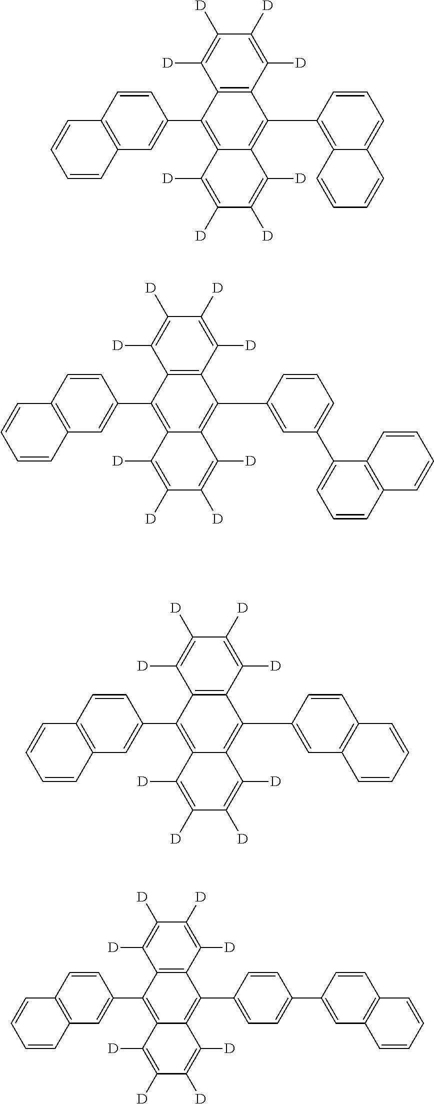 [SMILES: [2H]c1c([2H])c([2H])c2c(-c3cccc4ccccc34)c3c([2H])c([2H])c([2H])c([2H])c3c(-c3ccc4ccccc4c3)c2c1[2H] ; [2H]c1c([2H])c([2H])c2c(-c3ccc4ccccc4c3)c3c([2H])c([2H])c([2H])c([2H])c3c(-c3cccc(-c4cccc5ccccc45)c3)c2c1[2H] ; [2H]c1c([2H])c([2H])c2c(-c3ccc4ccccc4c3)c3c([2H])c([2H])c([2H])c([2H])c3c(-c3ccc4ccccc4c3)c2c1[2H] ; [2H]c1c([2H])c([2H])c2c(-c3ccc4ccccc4c3)c3c([2H])c([2H])c([2H])c([2H])c3c(-c3ccc(-c4ccc5ccccc5c4)cc3)c2c1[2H]]

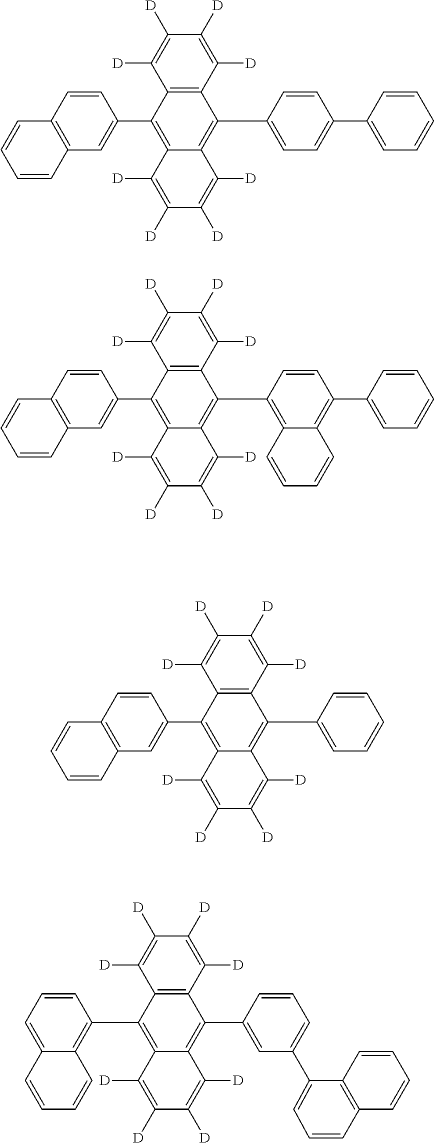 [SMILES: [2H]c1c([2H])c([2H])c2c(-c3ccc4ccccc4c3)c3c([2H])c([2H])c([2H])c([2H])c3c(-c3ccc(-c4ccccc4)cc3)c2c1[2H] ; [2H]c1c([2H])c([2H])c2c(-c3ccc(-c4ccccc4)c4ccccc34)c3c([2H])c([2H])c([2H])c([2H])c3c(-c3ccc4ccccc4c3)c2c1[2H] ; [2H]c1c([2H])c([2H])c2c(-c3ccc4ccccc4c3)c3c([2H])c([2H])c([2H])c([2H])c3c(-c3ccccc3)c2c1[2H] ; [2H]c1c([2H])c([2H])c2c(-c3cccc4ccccc34)c3c([2H])c([2H])c([2H])c([2H])c3c(-c3cccc(-c4cccc5ccccc45)c3)c2c1[2H]]

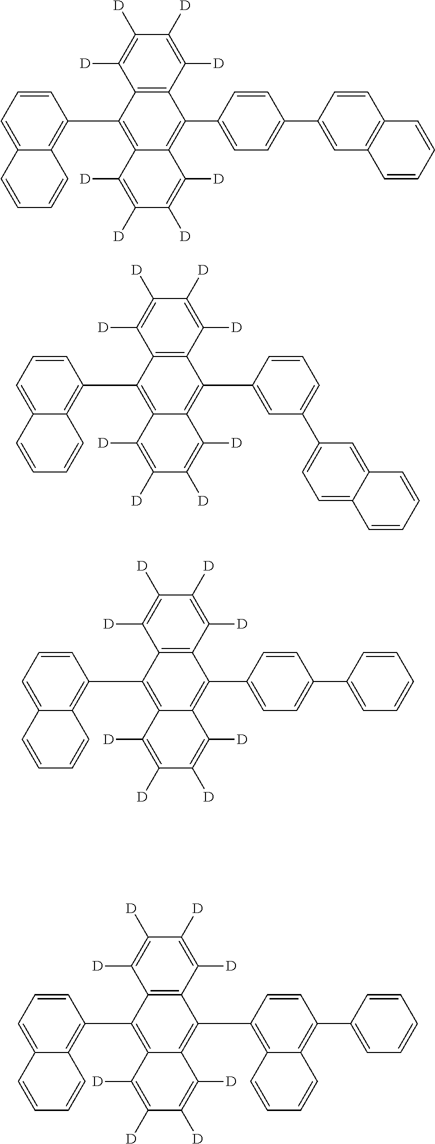

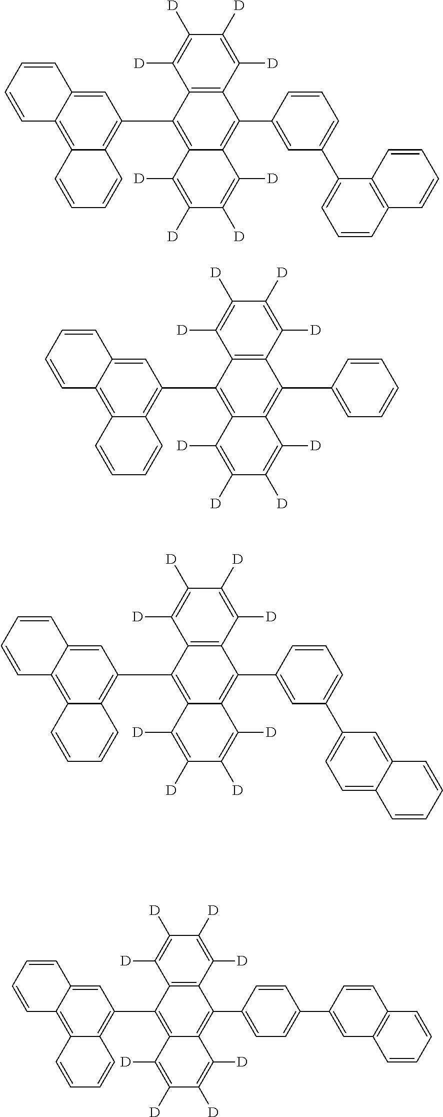

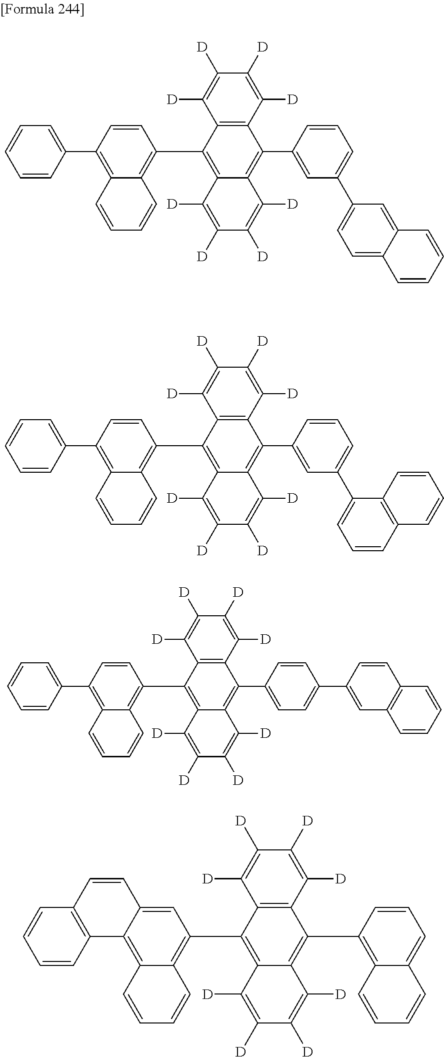

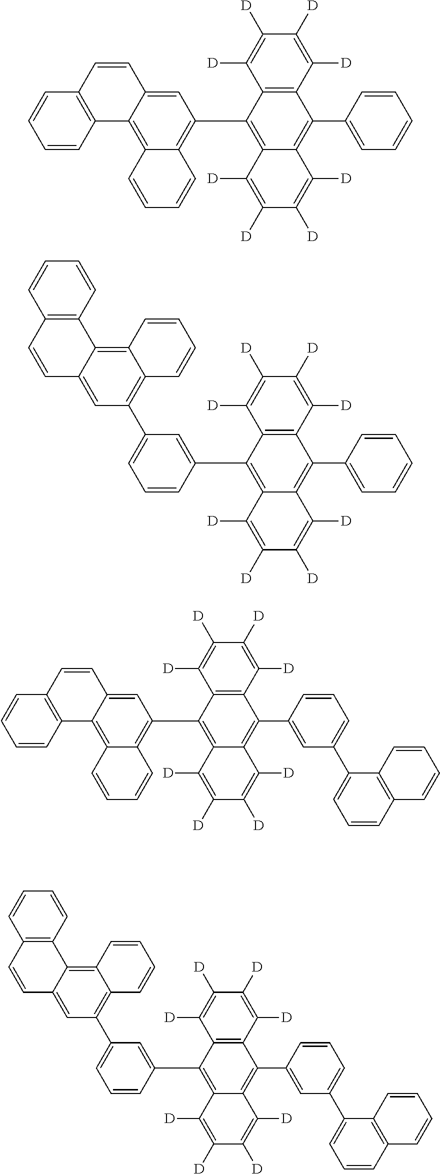

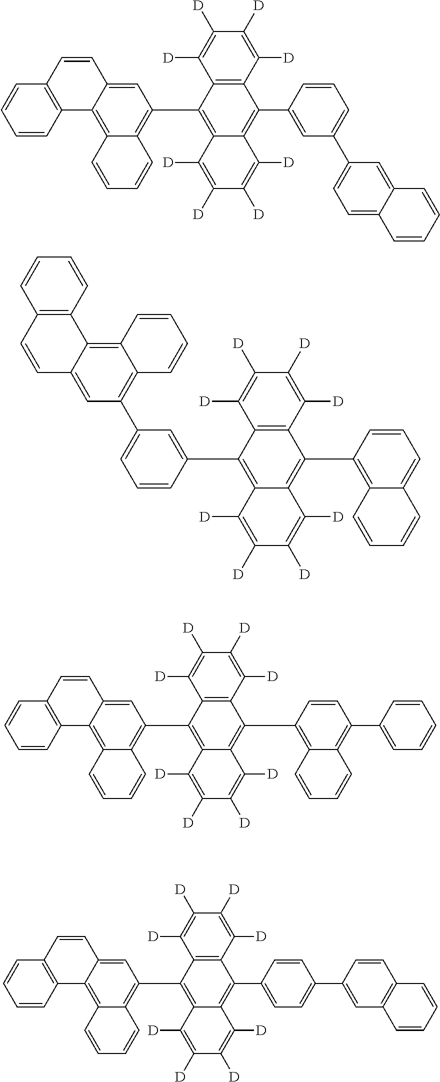 [SMILES: [2H]c1c([2H])c([2H])c2c(-c3cc4ccc5ccccc5c4c4ccccc34)c3c([2H])c([2H])c([2H])c([2H])c3c(-c3cccc(-c4ccc5ccccc5c4)c3)c2c1[2H] ; [2H]c1c([2H])c([2H])c2c(-c3cccc4ccccc34)c3c([2H])c([2H])c([2H])c([2H])c3c(-c3cccc(-c4cc5ccc6ccccc6c5c5ccccc45)c3)c2c1[2H] ; [2H]c1c([2H])c([2H])c2c(-c3cc4ccc5ccccc5c4c4ccccc34)c3c([2H])c([2H])c([2H])c([2H])c3c(-c3ccc(-c4ccccc4)c4ccccc34)c2c1[2H] ; [2H]c1c([2H])c([2H])c2c(-c3cc4ccc5ccccc5c4c4ccccc34)c3c([2H])c([2H])c([2H])c([2H])c3c(-c3ccc(-c4ccc5ccccc5c4)cc3)c2c1[2H]]

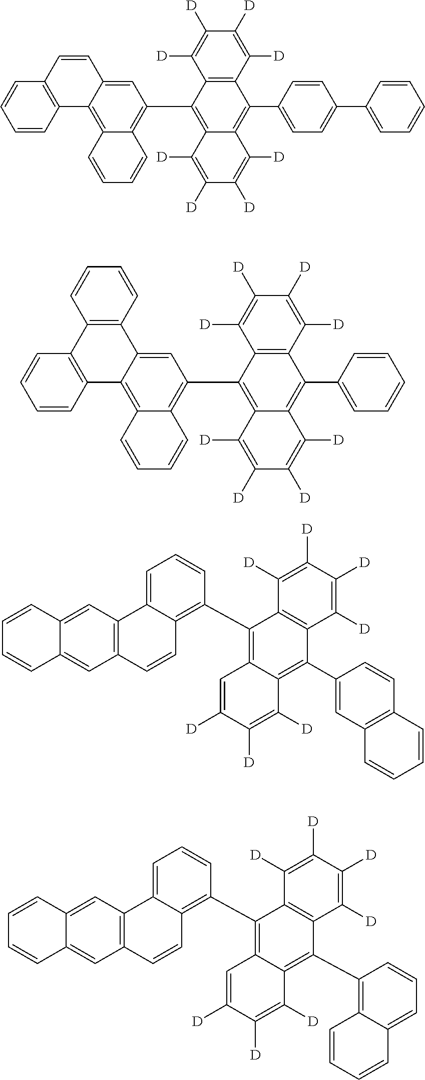

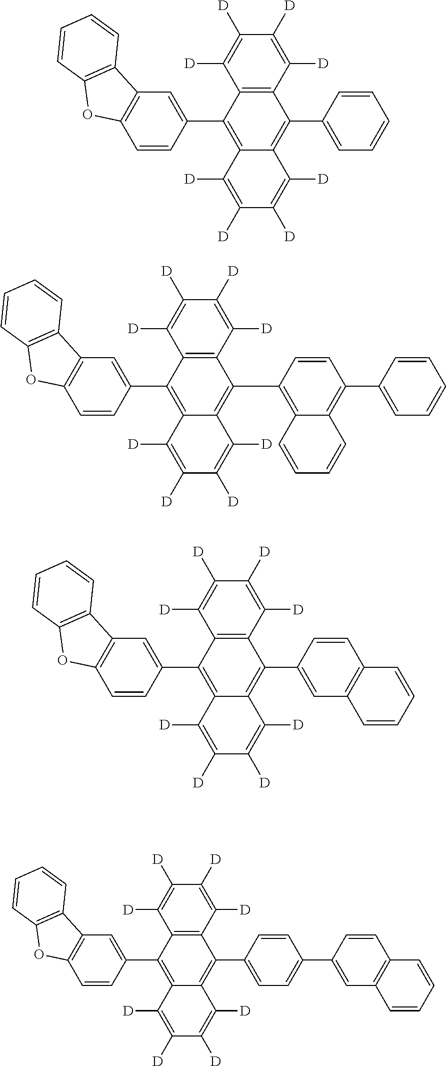

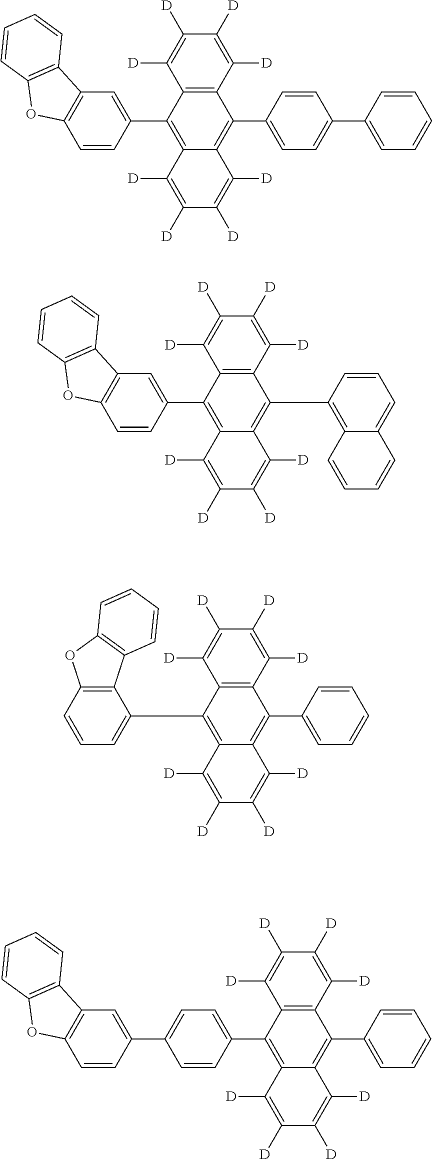 [SMILES: [2H]c1c([2H])c([2H])c2c(-c3ccc4oc5ccccc5c4c3)c3c([2H])c([2H])c([2H])c([2H])c3c(-c3ccc(-c4ccccc4)cc3)c2c1[2H] ; [2H]c1c([2H])c([2H])c2c(-c3cccc4ccccc34)c3c([2H])c([2H])c([2H])c([2H])c3c(-c3ccc4oc5ccccc5c4c3)c2c1[2H] ; [2H]c1c([2H])c([2H])c2c(-c3cccc4oc5ccccc5c34)c3c([2H])c([2H])c([2H])c([2H])c3c(-c3ccccc3)c2c1[2H] ; [2H]c1c([2H])c([2H])c2c(-c3ccc(-c4ccc5oc6ccccc6c5c4)cc3)c3c([2H])c([2H])c([2H])c([2H])c3c(-c3ccccc3)c2c1[2H]]

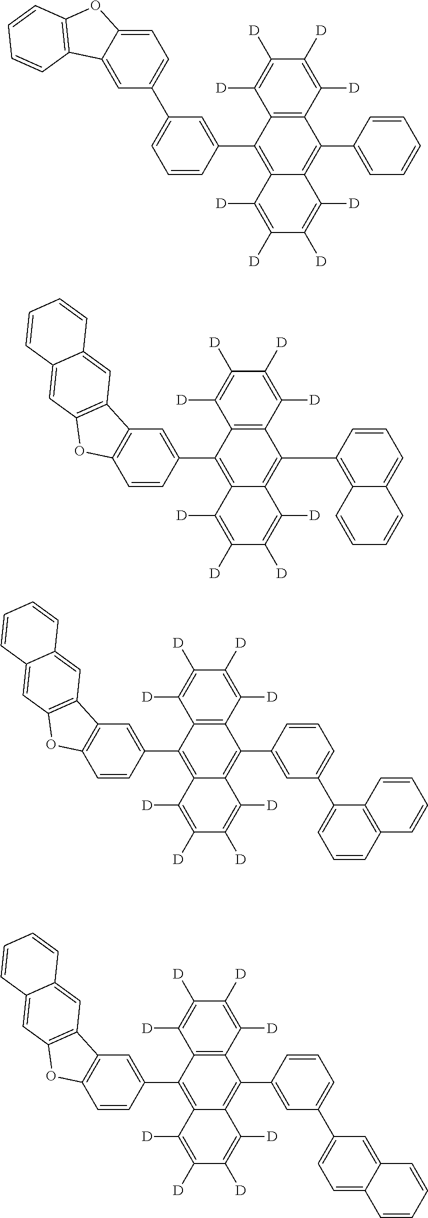 [SMILES: [2H]c1c([2H])c([2H])c2c(-c3cccc(-c4ccc5oc6ccccc6c5c4)c3)c3c([2H])c([2H])c([2H])c([2H])c3c(-c3ccccc3)c2c1[2H] ; [2H]c1c([2H])c([2H])c2c(-c3cccc4ccccc34)c3c([2H])c([2H])c([2H])c([2H])c3c(-c3ccc4oc5cc6ccccc6cc5c4c3)c2c1[2H] ; [2H]c1c([2H])c([2H])c2c(-c3ccc4oc5cc6ccccc6cc5c4c3)c3c([2H])c([2H])c([2H])c([2H])c3c(-c3cccc(-c4cccc5ccccc45)c3)c2c1[2H] ; [2H]c1c([2H])c([2H])c2c(-c3ccc4oc5cc6ccccc6cc5c4c3)c3c([2H])c([2H])c([2H])c([2H])c3c(-c3cccc(-c4ccc5ccccc5c4)c3)c2c1[2H]]

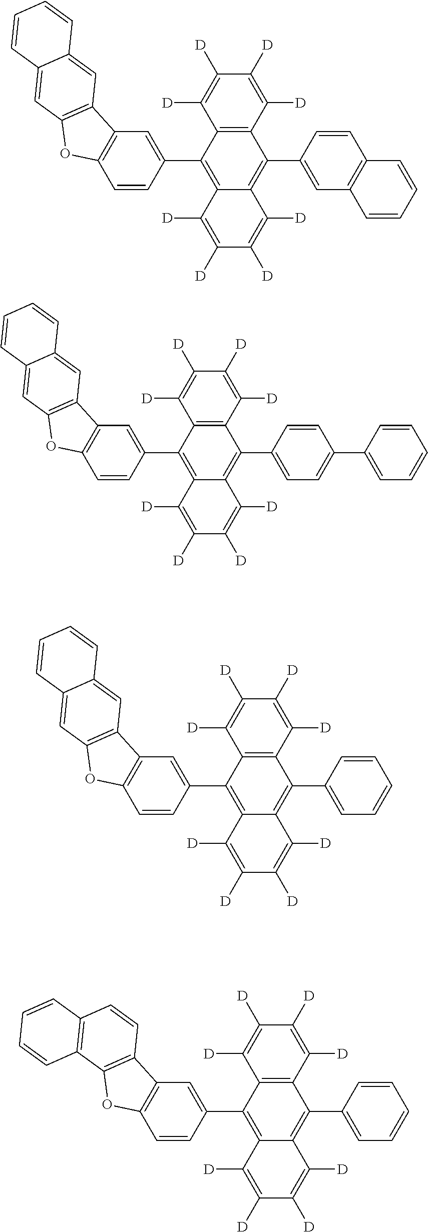 [SMILES: [2H]c1c([2H])c([2H])c2c(-c3ccc4oc5cc6ccccc6cc5c4c3)c3c([2H])c([2H])c([2H])c([2H])c3c(-c3ccc4ccccc4c3)c2c1[2H] ; [2H]c1c([2H])c([2H])c2c(-c3ccc4oc5cc6ccccc6cc5c4c3)c3c([2H])c([2H])c([2H])c([2H])c3c(-c3ccc(-c4ccccc4)cc3)c2c1[2H] ; [2H]c1c([2H])c([2H])c2c(-c3ccc4oc5cc6ccccc6cc5c4c3)c3c([2H])c([2H])c([2H])c([2H])c3c(-c3ccccc3)c2c1[2H] ; [2H]c1c([2H])c([2H])c2c(-c3ccc4oc5c6ccccc6ccc5c4c3)c3c([2H])c([2H])c([2H])c([2H])c3c(-c3ccccc3)c2c1[2H]]

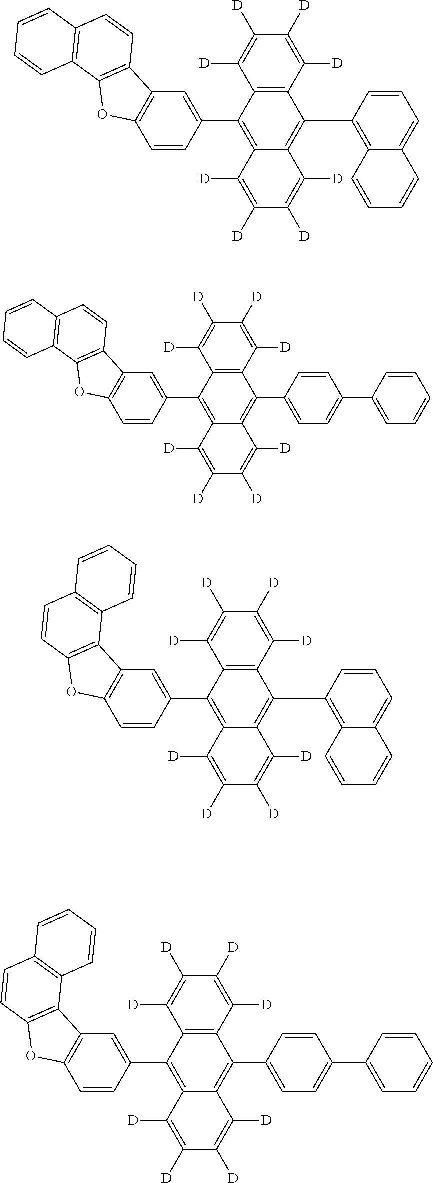 [SMILES: [2H]c1c([2H])c([2H])c2c(-c3cccc4ccccc34)c3c([2H])c([2H])c([2H])c([2H])c3c(-c3ccc4oc5c6ccccc6ccc5c4c3)c2c1[2H] ; [2H]c1c([2H])c([2H])c2c(-c3ccc4oc5c6ccccc6ccc5c4c3)c3c([2H])c([2H])c([2H])c([2H])c3c(-c3ccc(-c4ccccc4)cc3)c2c1[2H] ; [2H]c1c([2H])c([2H])c2c(-c3cccc4ccccc34)c3c([2H])c([2H])c([2H])c([2H])c3c(-c3ccc4oc5ccc6ccccc6c5c4c3)c2c1[2H] ; [2H]c1c([2H])c([2H])c2c(-c3ccc4oc5ccc6ccccc6c5c4c3)c3c([2H])c([2H])c([2H])c([2H])c3c(-c3ccc(-c4ccccc4)cc3)c2c1[2H]]

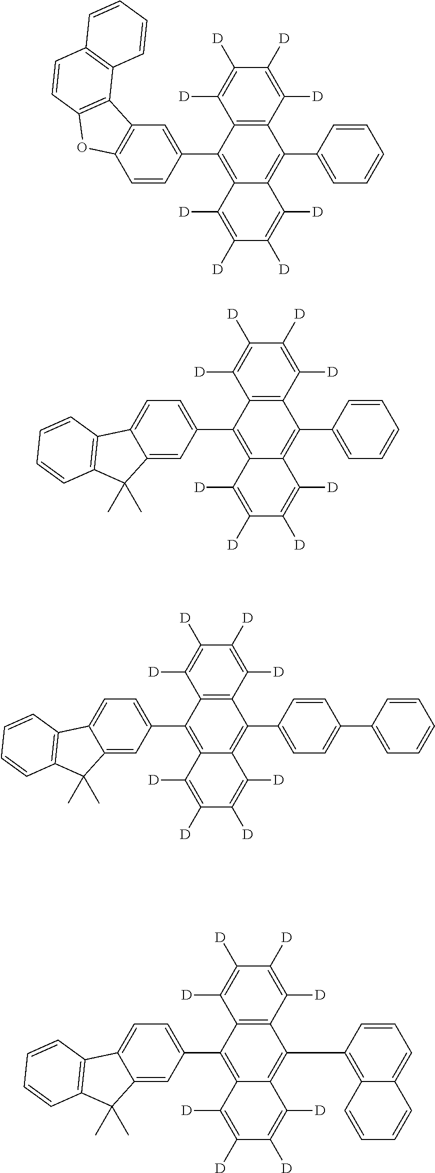 [SMILES: [2H]c1c([2H])c([2H])c2c(-c3ccc4oc5ccc6ccccc6c5c4c3)c3c([2H])c([2H])c([2H])c([2H])c3c(-c3ccccc3)c2c1[2H] ; [2H]c1c([2H])c([2H])c2c(-c3ccc4c(c3)C(C)(C)c3ccccc3-4)c3c([2H])c([2H])c([2H])c([2H])c3c(-c3ccccc3)c2c1[2H] ; [2H]c1c([2H])c([2H])c2c(-c3ccc4c(c3)C(C)(C)c3ccccc3-4)c3c([2H])c([2H])c([2H])c([2H])c3c(-c3ccc(-c4ccccc4)cc3)c2c1[2H] ; [2H]c1c([2H])c([2H])c2c(-c3cccc4ccccc34)c3c([2H])c([2H])c([2H])c([2H])c3c(-c3ccc4c(c3)C(C)(C)c3ccccc3-4)c2c1[2H]]

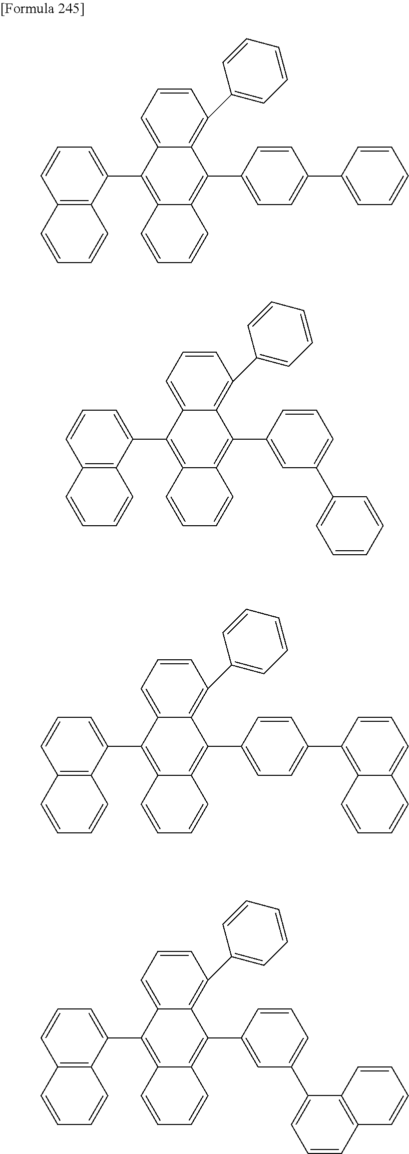

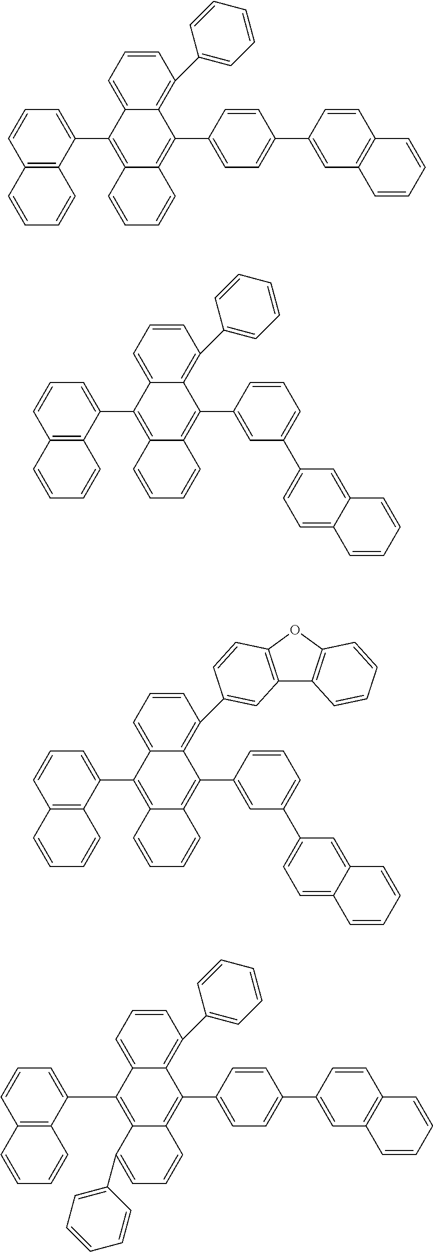

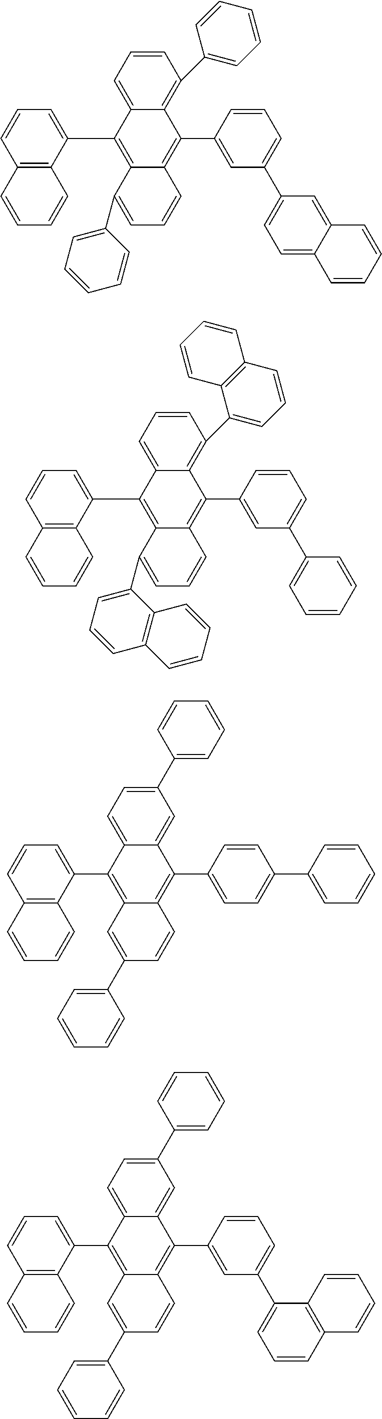

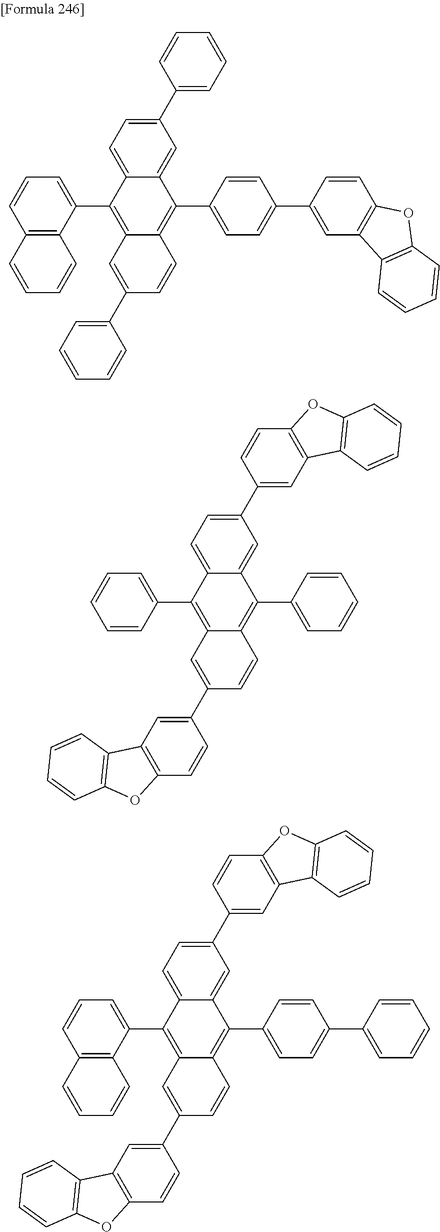 [SMILES: c1ccc(-c2ccc3c(-c4cccc5ccccc45)c4cc(-c5ccccc5)ccc4c(-c4ccc(-c5ccc6oc7ccccc7c6c5)cc4)c3c2)cc1 ; c1ccc(-c2c3ccc(-c4ccc5oc6ccccc6c5c4)cc3c(-c3ccccc3)c3ccc(-c4ccc5oc6ccccc6c5c4)cc23)cc1 ; c1ccc(-c2ccc(-c3c4cc(-c5ccc6oc7ccccc7c6c5)ccc4c(-c4cccc5ccccc45)c4cc(-c5ccc6oc7ccccc7c6c5)ccc34)cc2)cc1]

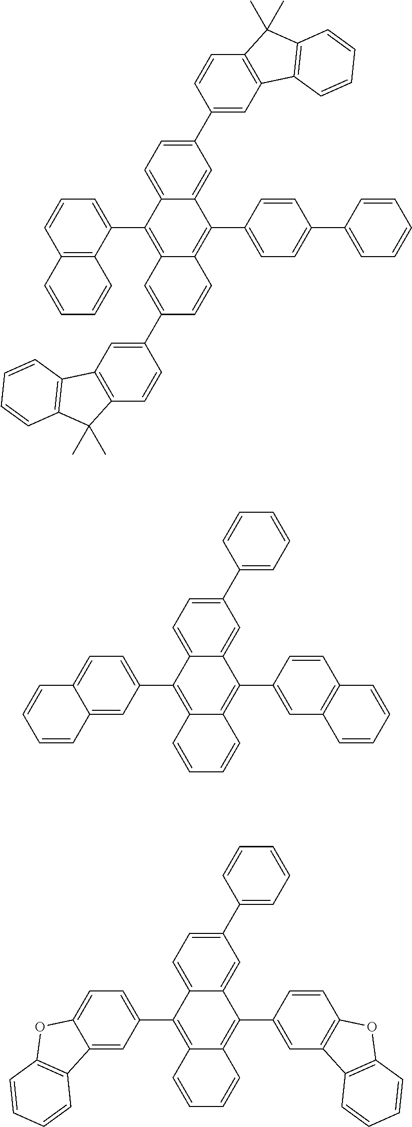

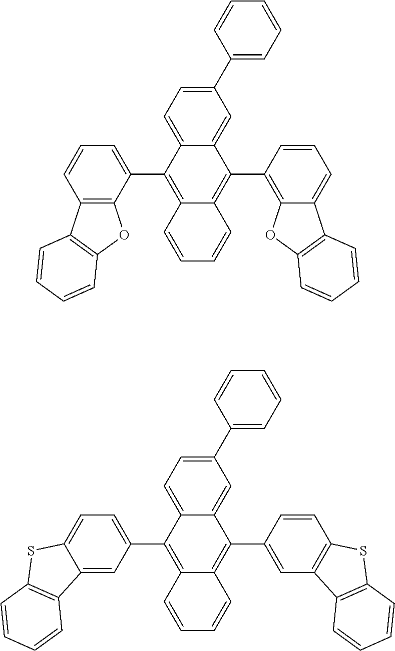 [SMILES: c1ccc(-c2ccc3c(-c4cccc5c4oc4ccccc45)c4ccccc4c(-c4cccc5c4oc4ccccc45)c3c2)cc1 ; c1ccc(-c2ccc3c(-c4ccc5sc6ccccc6c5c4)c4ccccc4c(-c4ccc5sc6ccccc6c5c4)c3c2)cc1]

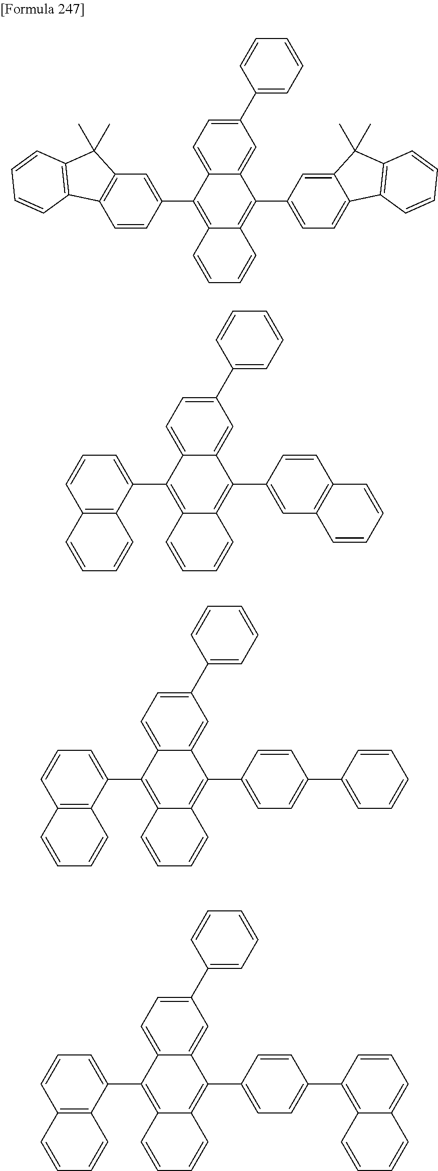

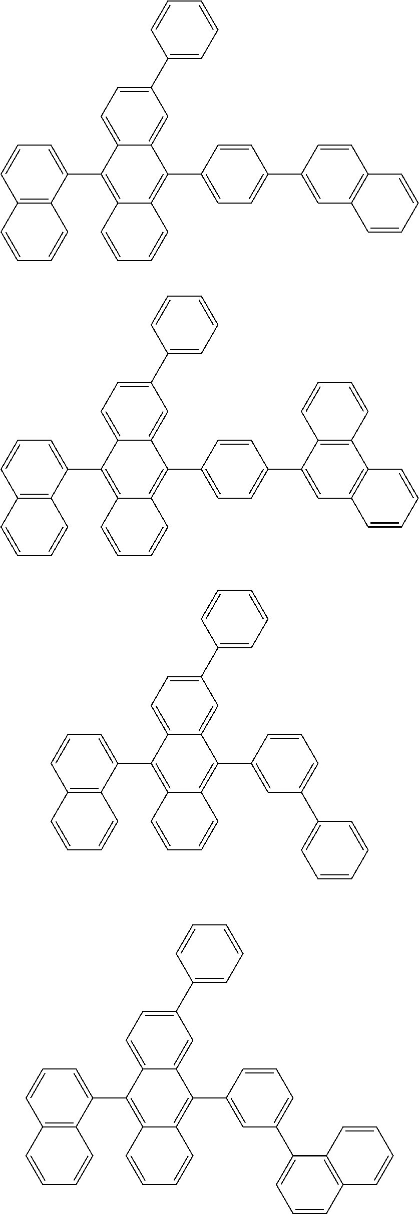

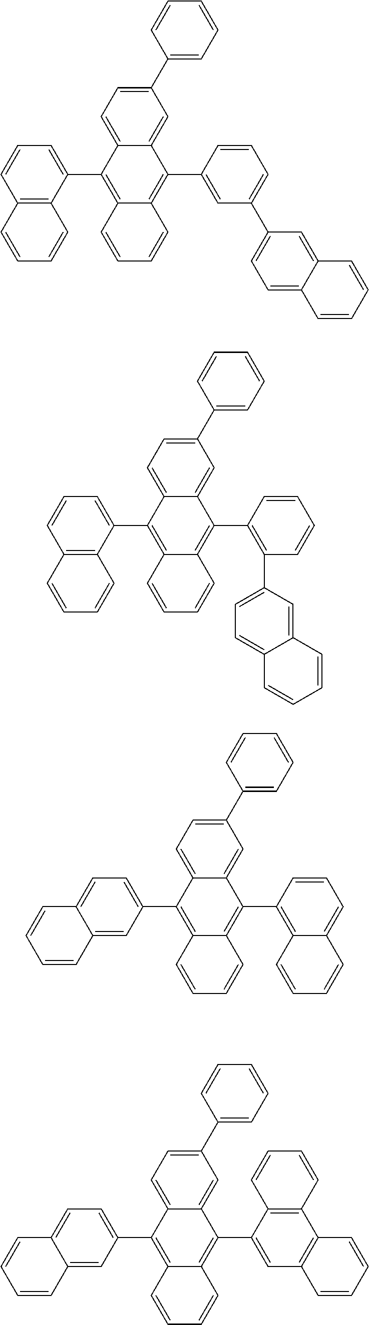

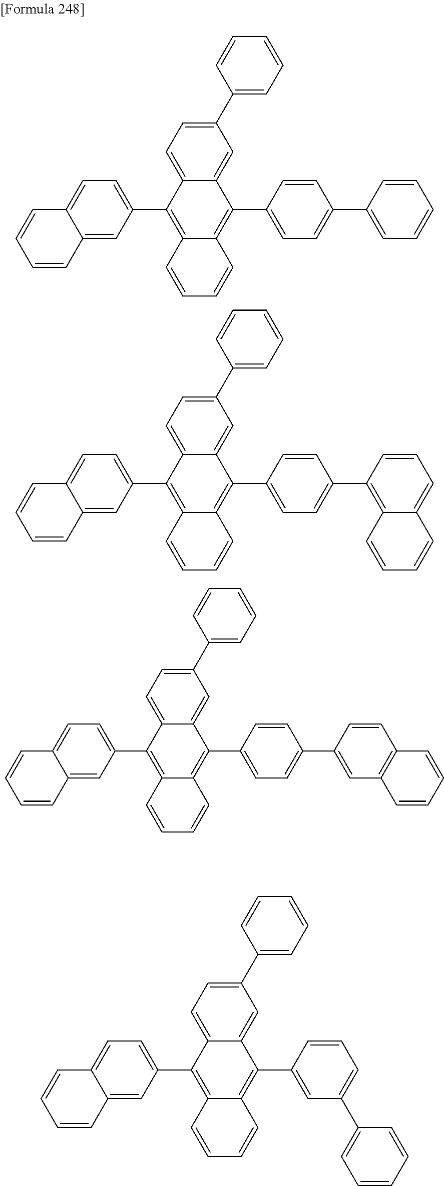

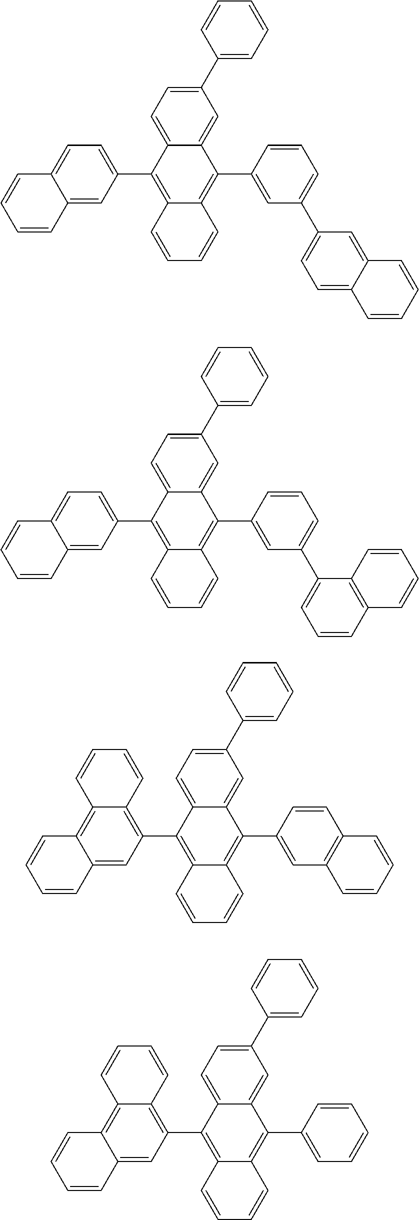

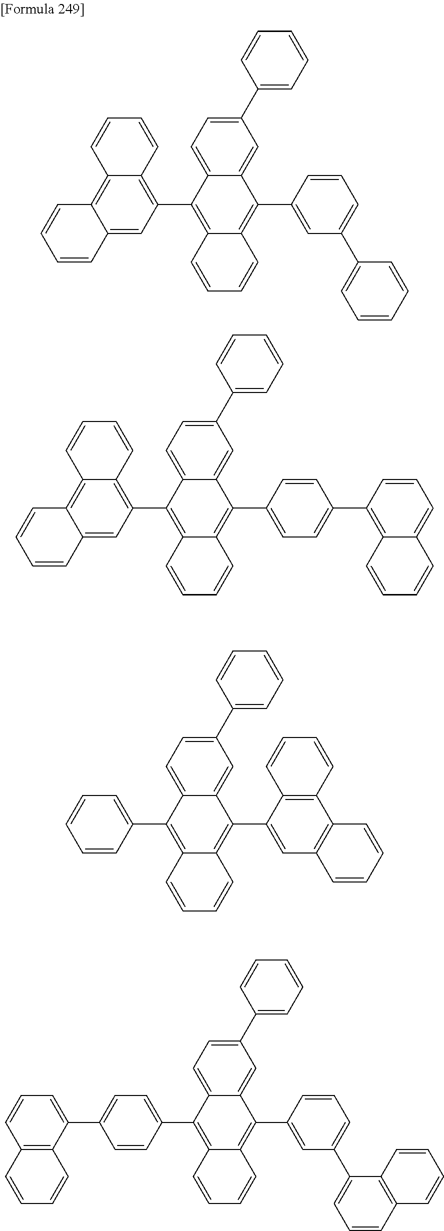

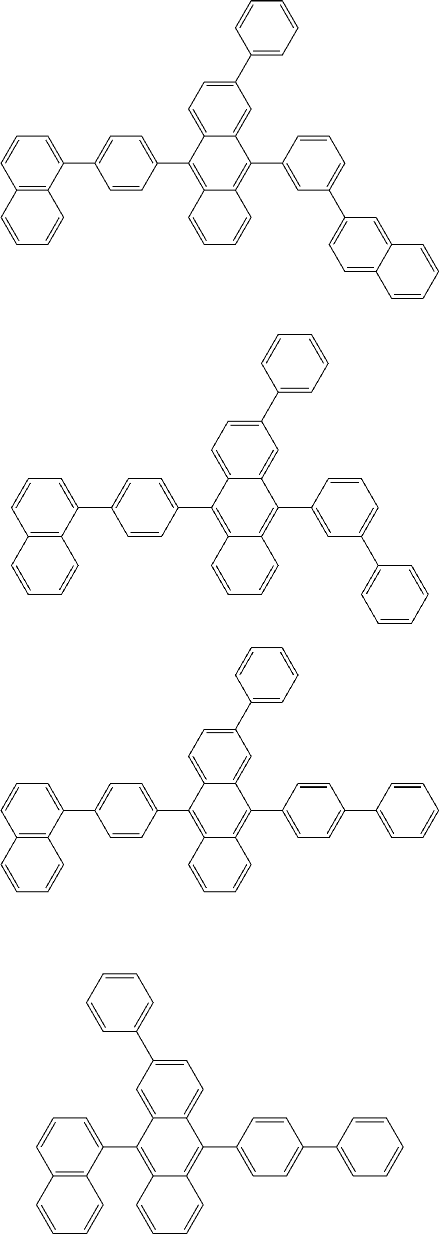

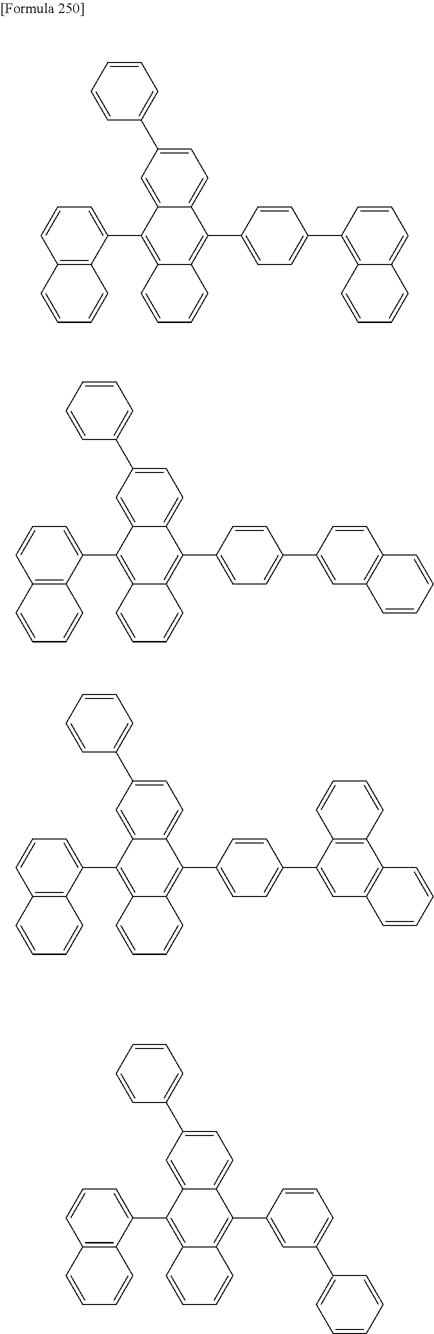

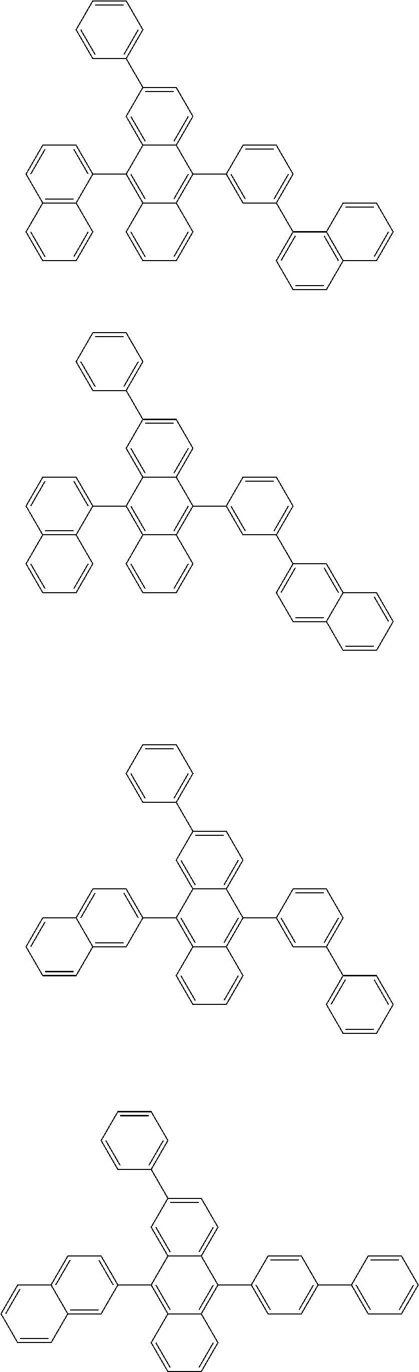

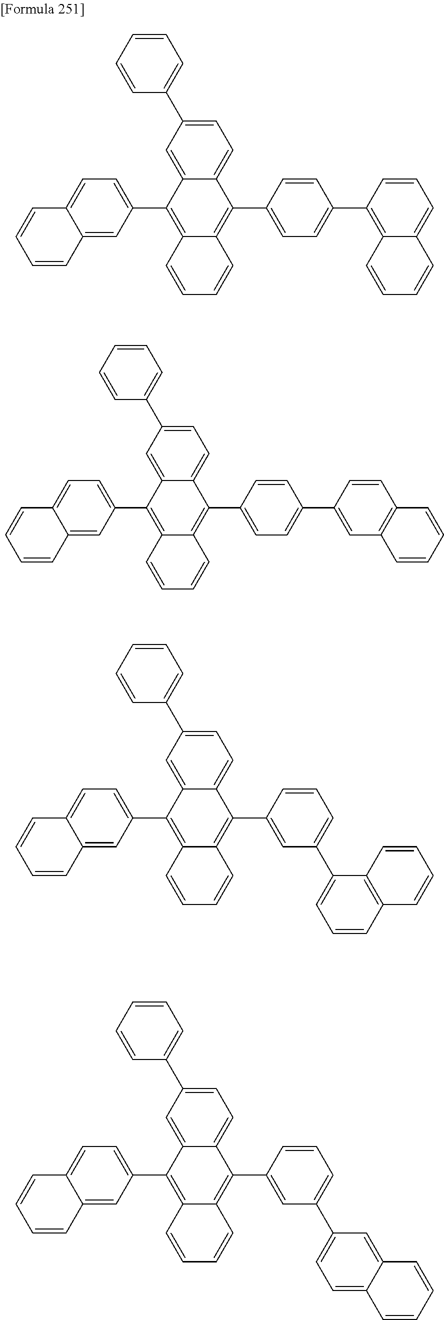

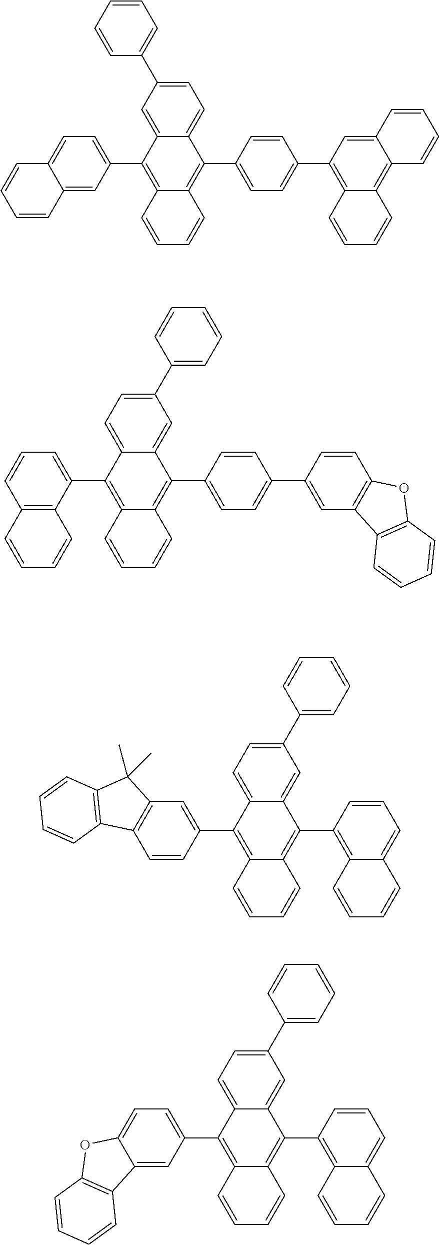

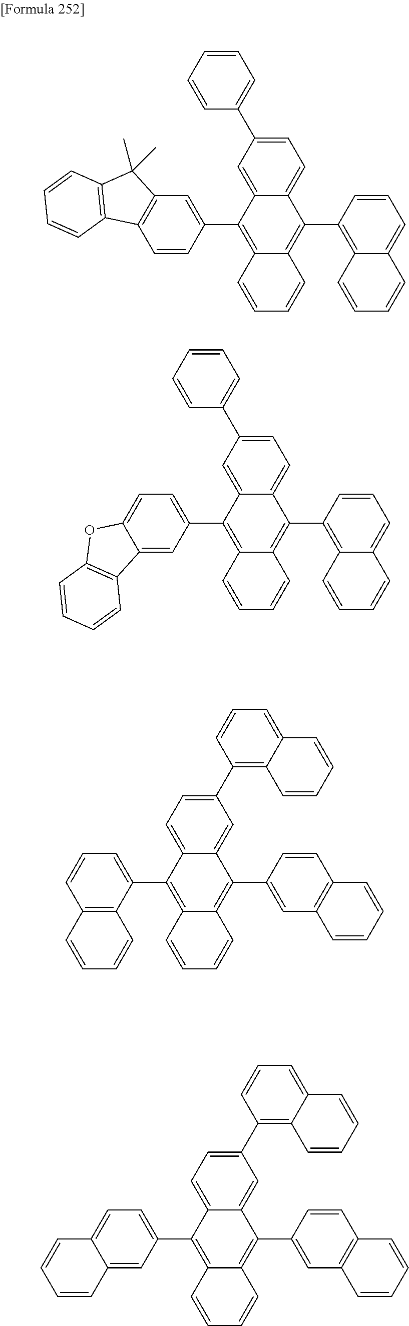

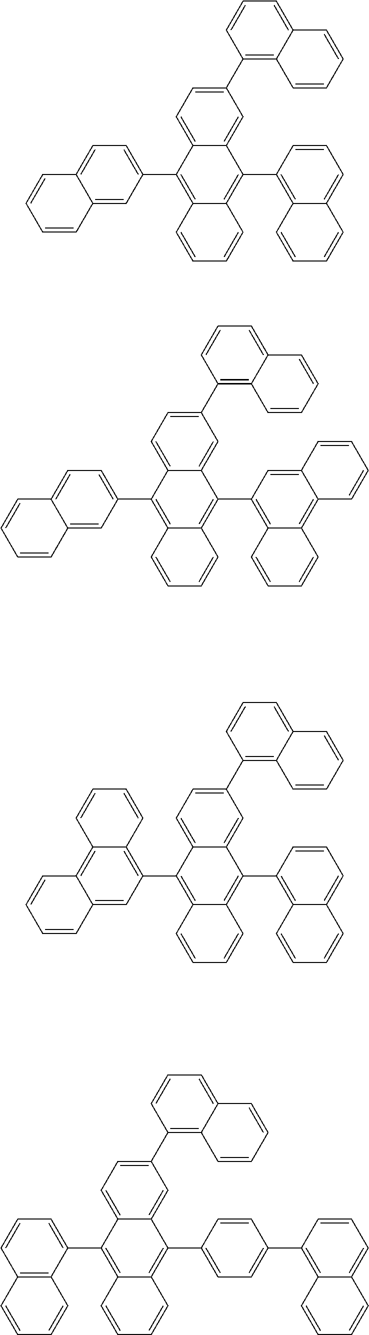

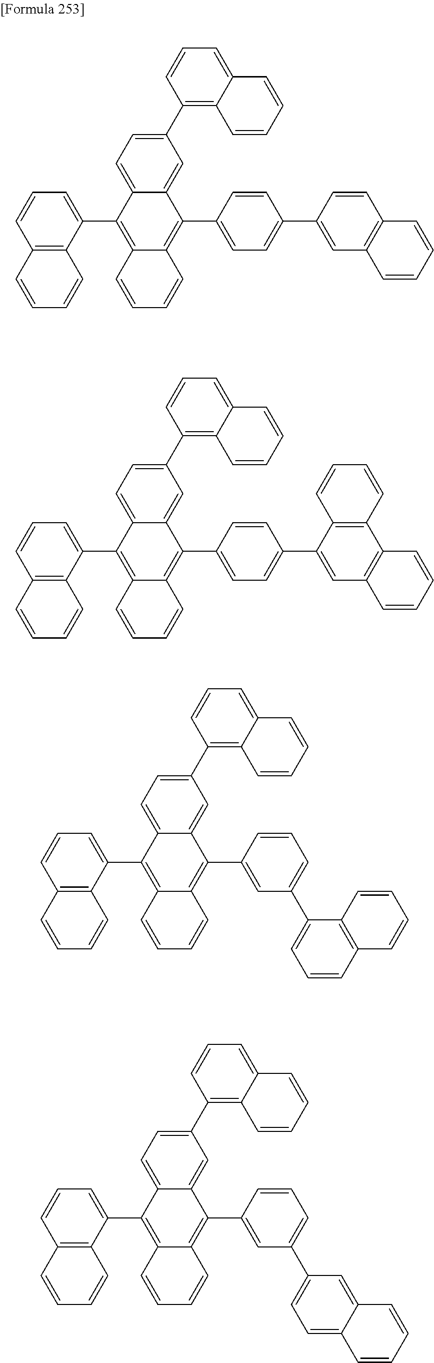

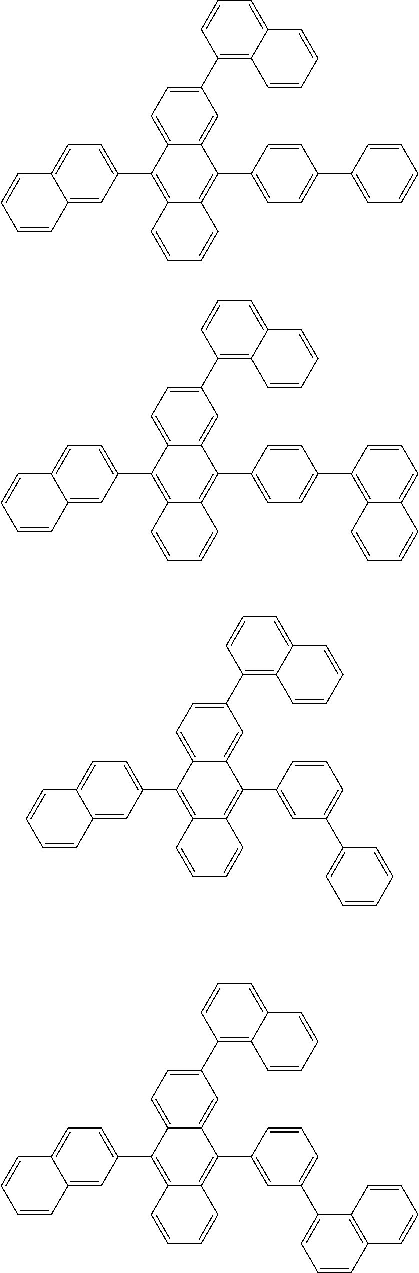

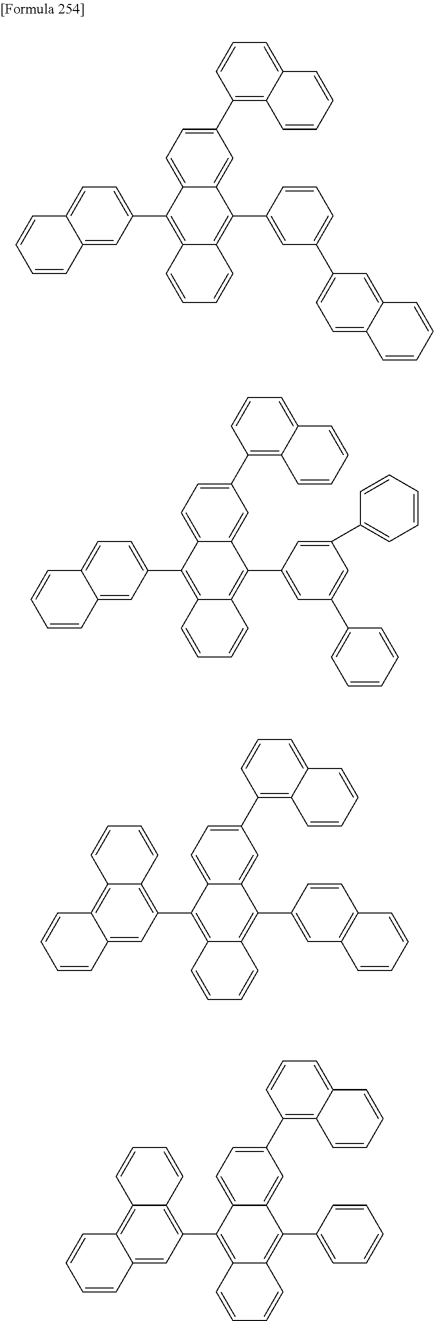

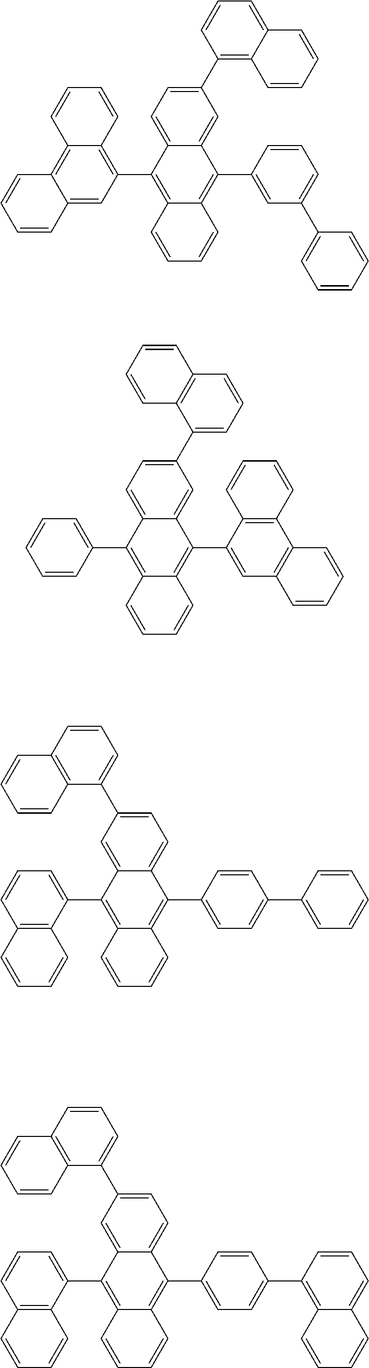

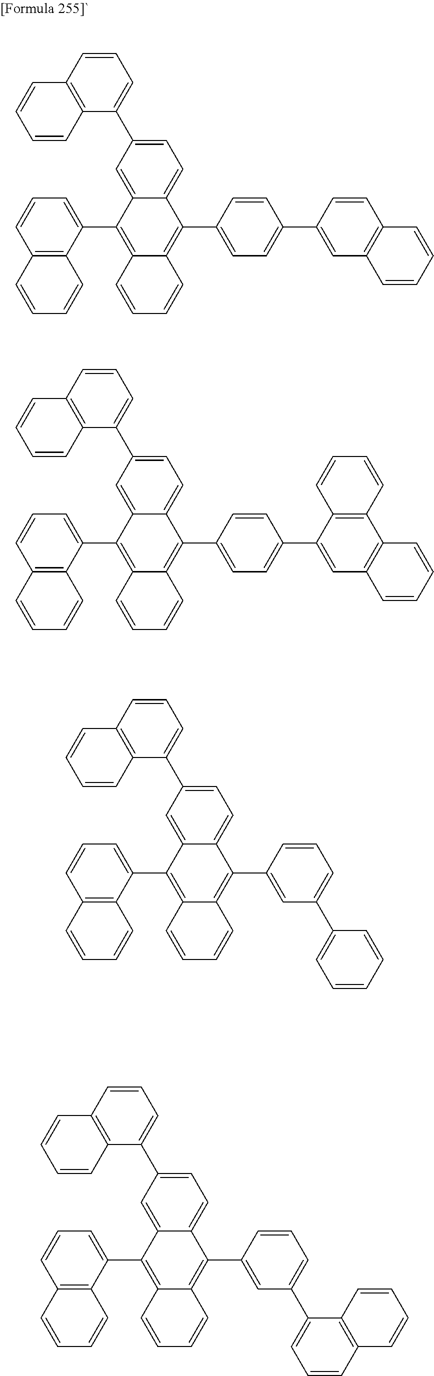

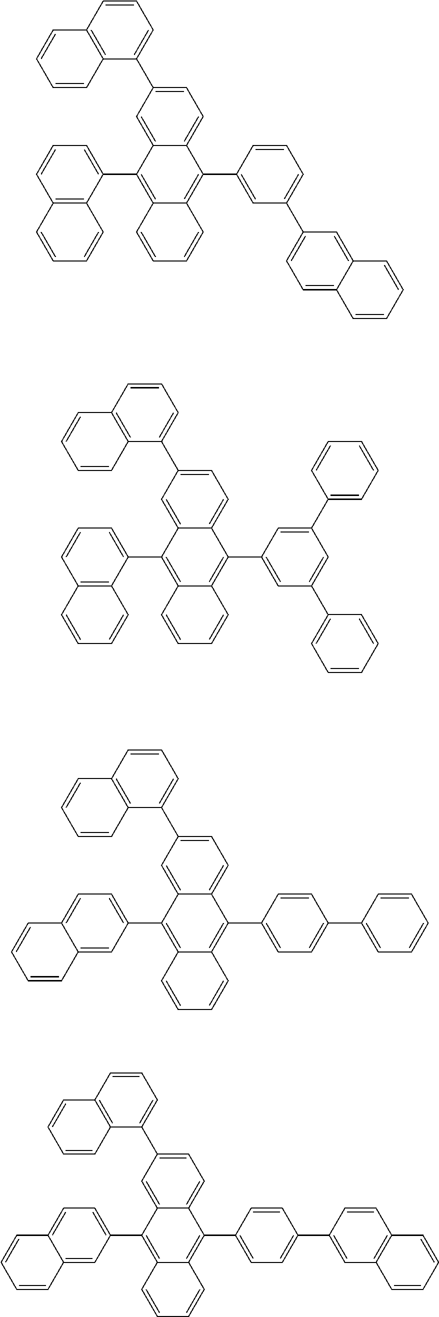

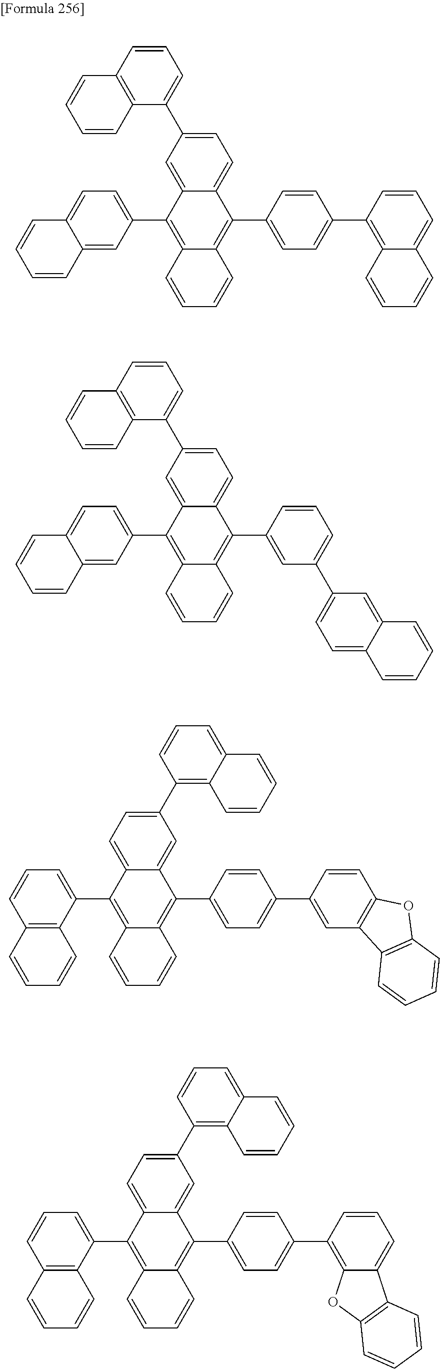

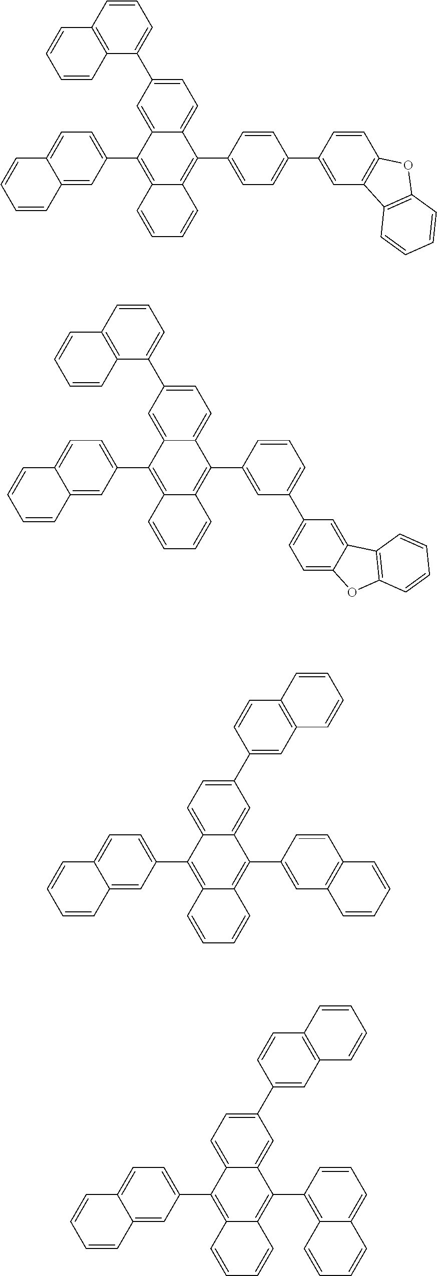

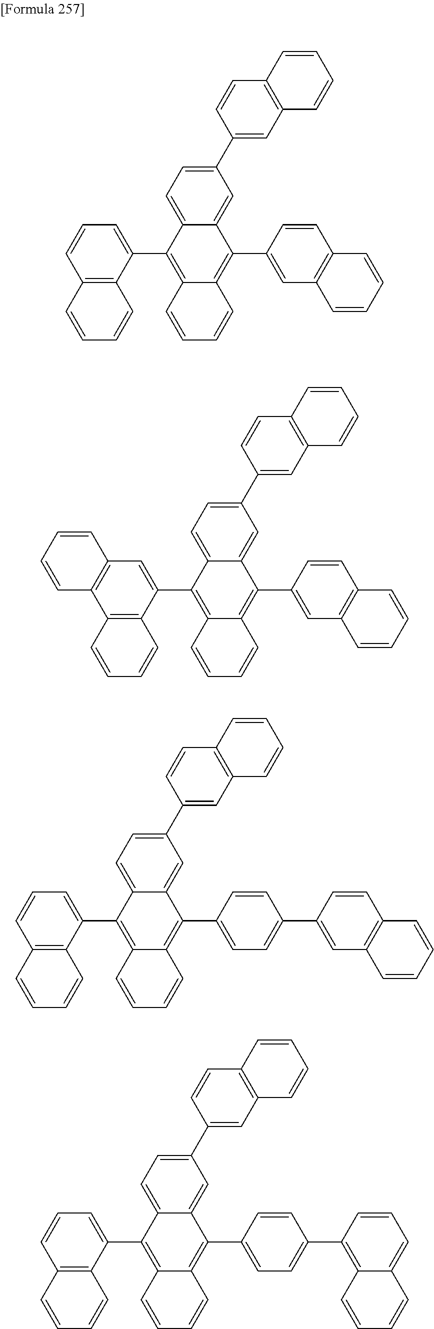

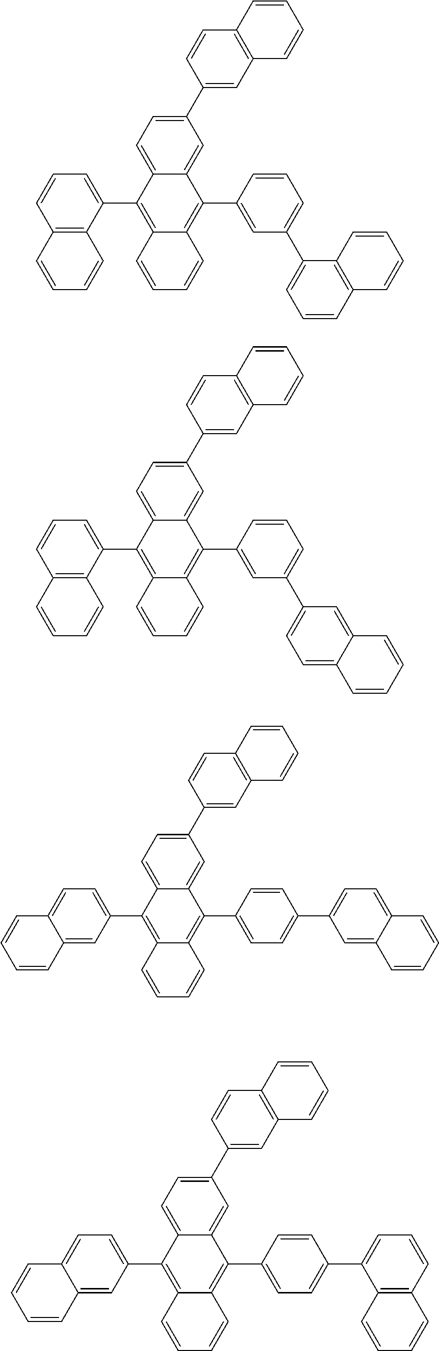

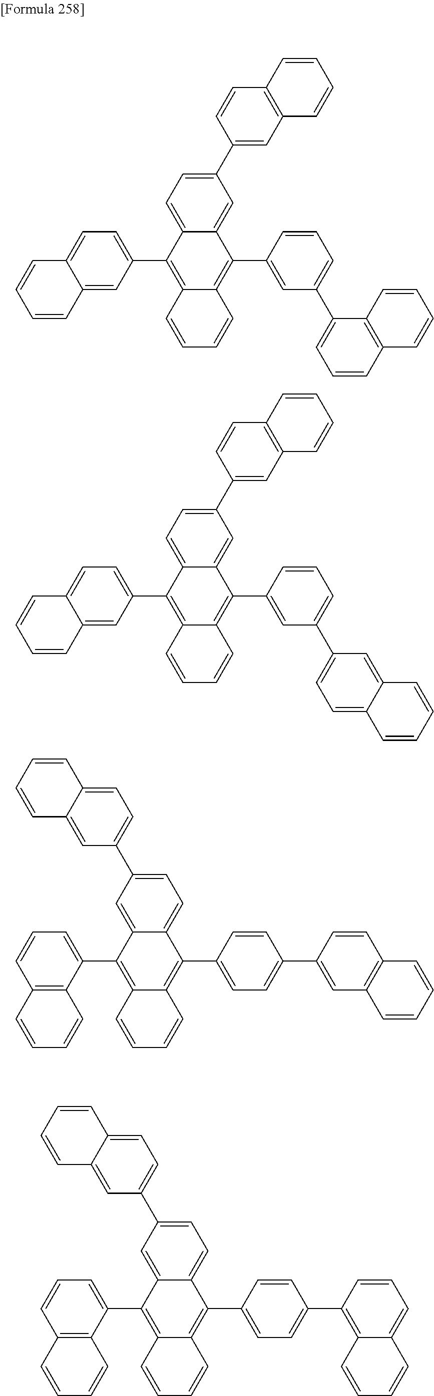

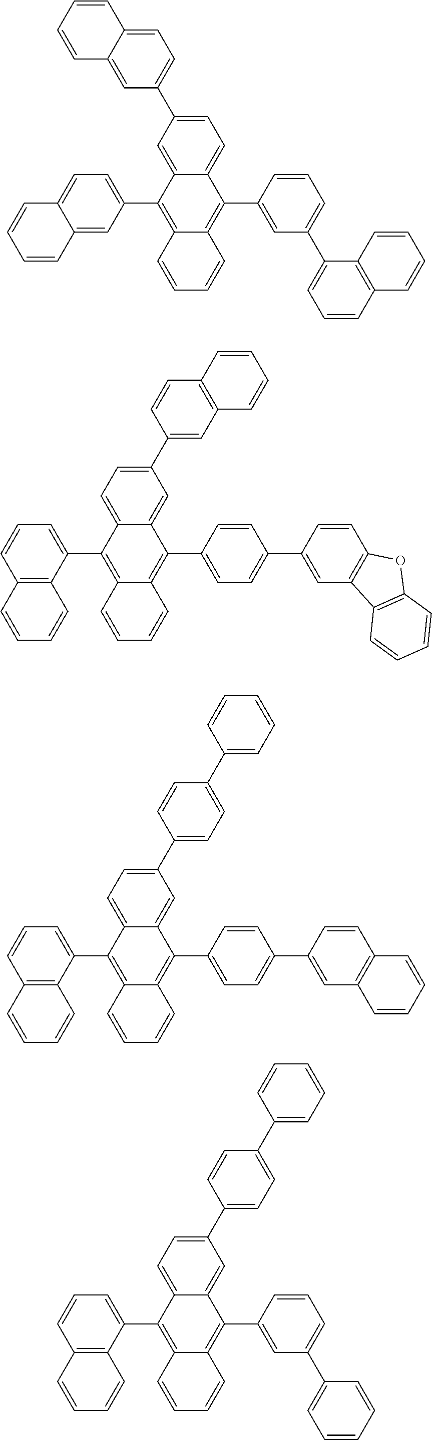

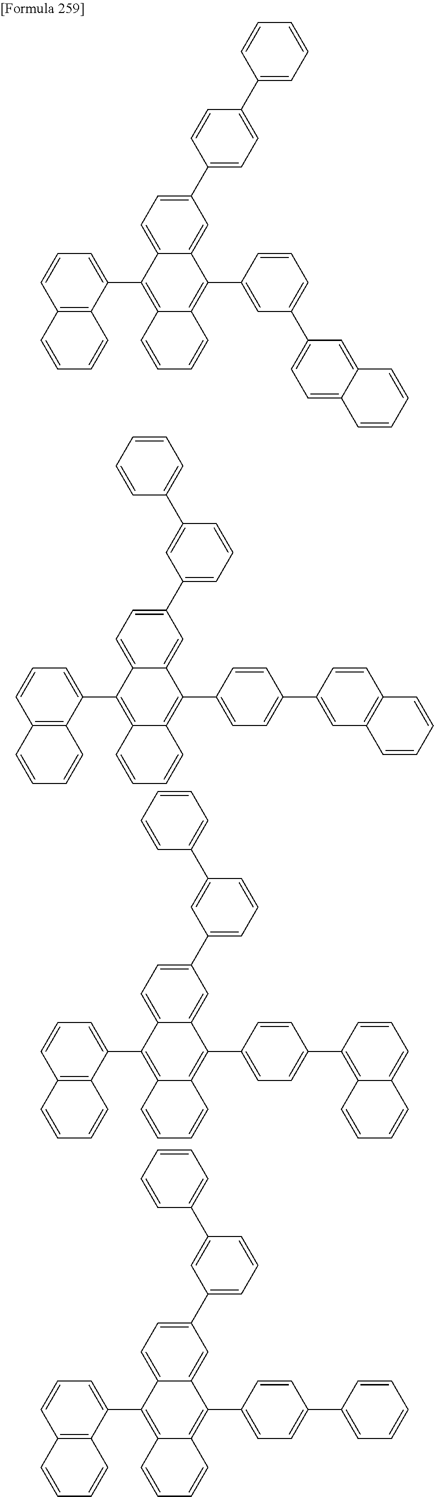

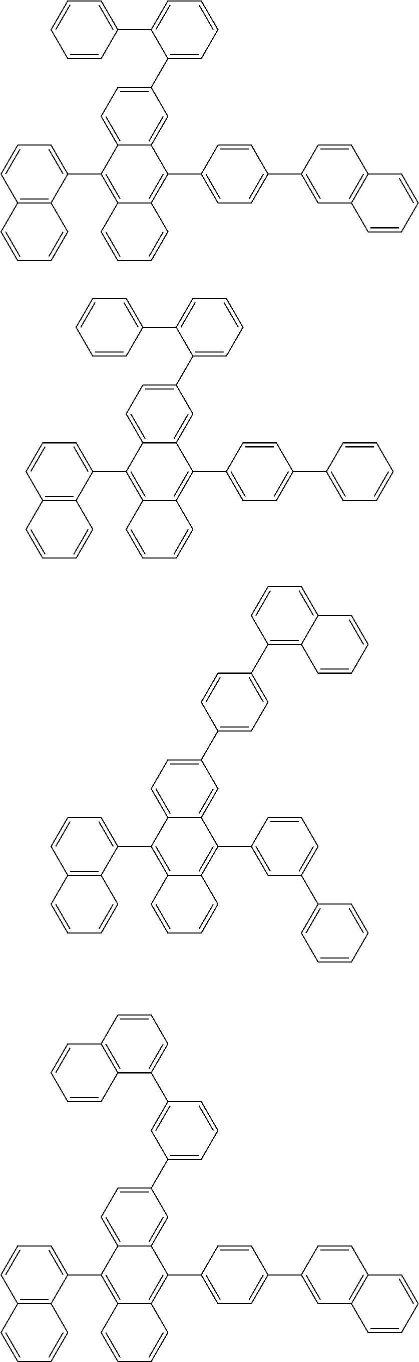

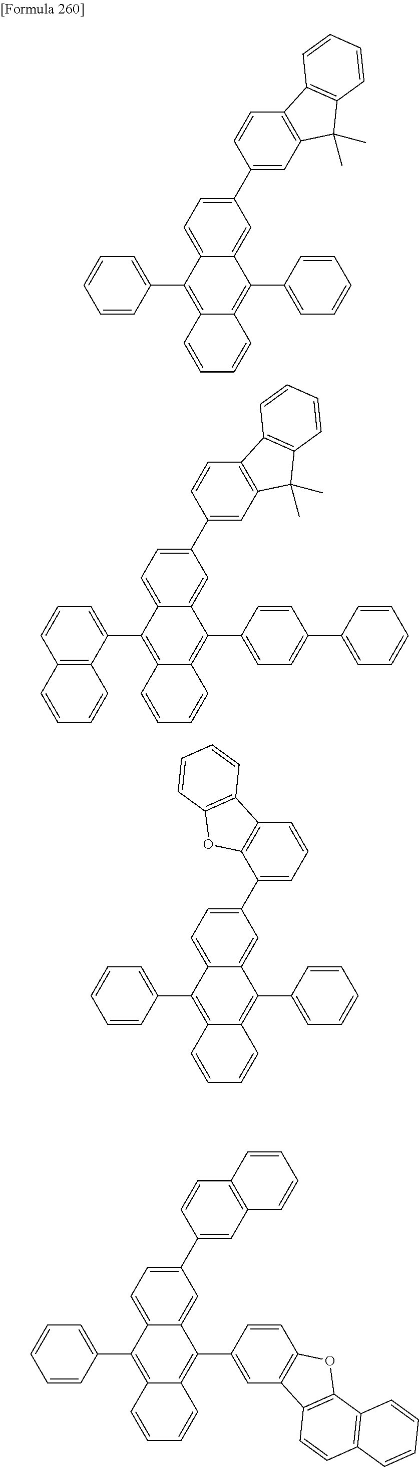

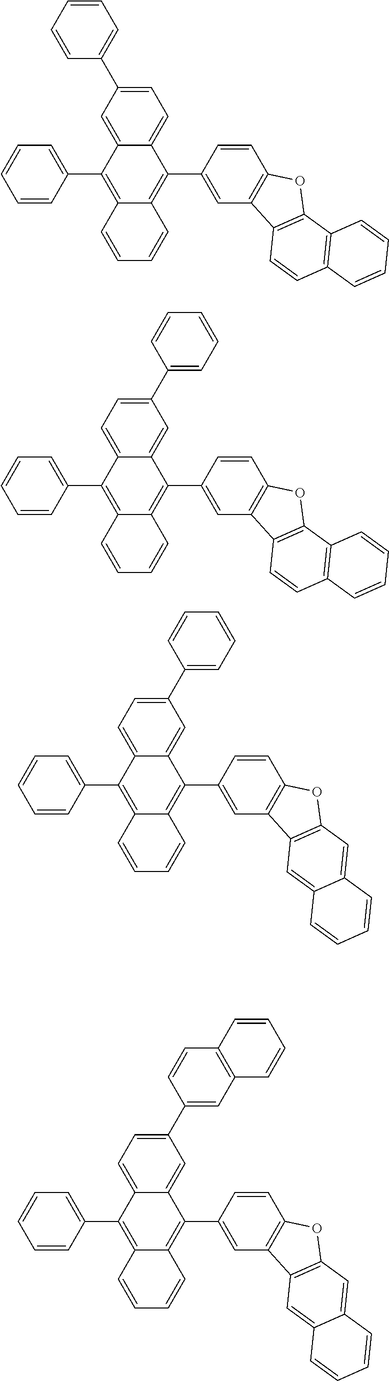 [SMILES: c1ccc(-c2ccc3c(-c4ccc5oc6c7ccccc7ccc6c5c4)c4ccccc4c(-c4ccccc4)c3c2)cc1 ; c1ccc(-c2ccc3c(-c4ccccc4)c4ccccc4c(-c4ccc5oc6c7ccccc7ccc6c5c4)c3c2)cc1 ; c1ccc(-c2ccc3c(-c4ccccc4)c4ccccc4c(-c4ccc5oc6cc7ccccc7cc6c5c4)c3c2)cc1 ; c1ccc(-c2c3ccccc3c(-c3ccc4oc5cc6ccccc6cc5c4c3)c3cc(-c4ccc5ccccc5c4)ccc23)cc1]

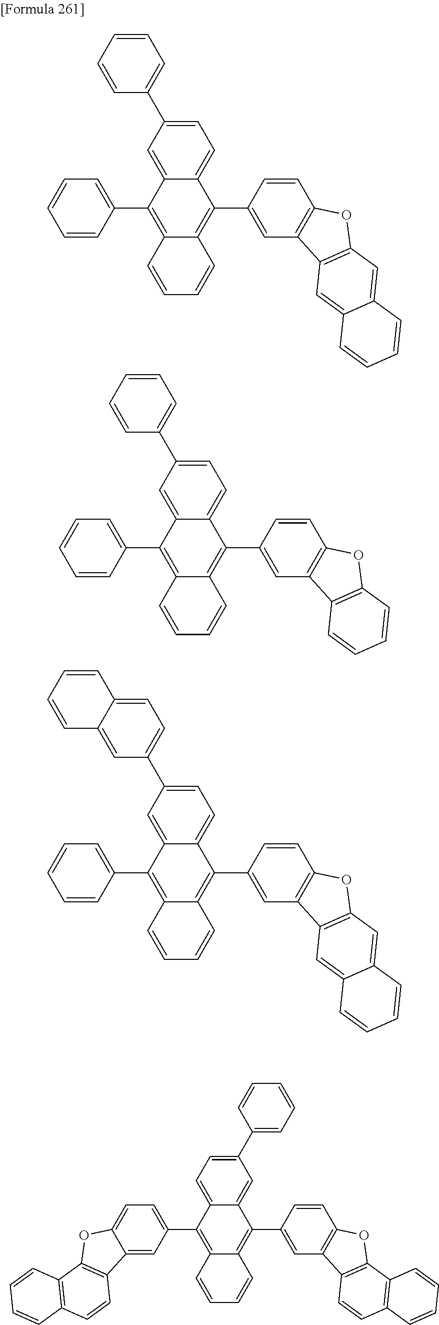 [SMILES: c1ccc(-c2ccc3c(-c4ccc5oc6cc7ccccc7cc6c5c4)c4ccccc4c(-c4ccccc4)c3c2)cc1 ; c1ccc(-c2ccc3c(-c4ccc5oc6ccccc6c5c4)c4ccccc4c(-c4ccccc4)c3c2)cc1 ; c1ccc(-c2c3ccccc3c(-c3ccc4oc5cc6ccccc6cc5c4c3)c3ccc(-c4ccc5ccccc5c4)cc23)cc1 ; c1ccc(-c2ccc3c(-c4ccc5oc6c7ccccc7ccc6c5c4)c4ccccc4c(-c4ccc5oc6c7ccccc7ccc6c5c4)c3c2)cc1]

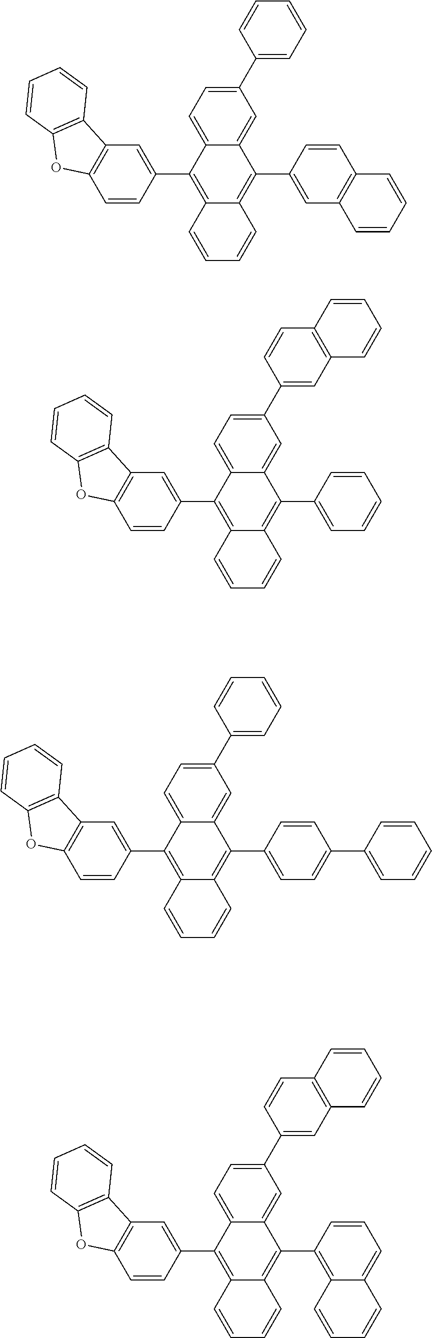 [SMILES: c1ccc(-c2ccc3c(-c4ccc5oc6ccccc6c5c4)c4ccccc4c(-c4ccc5ccccc5c4)c3c2)cc1 ; c1ccc(-c2c3ccccc3c(-c3ccc4oc5ccccc5c4c3)c3ccc(-c4ccc5ccccc5c4)cc23)cc1 ; c1ccc(-c2ccc(-c3c4ccccc4c(-c4ccc5oc6ccccc6c5c4)c4ccc(-c5ccccc5)cc34)cc2)cc1 ; c1ccc2cc(-c3ccc4c(-c5ccc6oc7ccccc7c6c5)c5ccccc5c(-c5cccc6ccccc56)c4c3)ccc2c1]

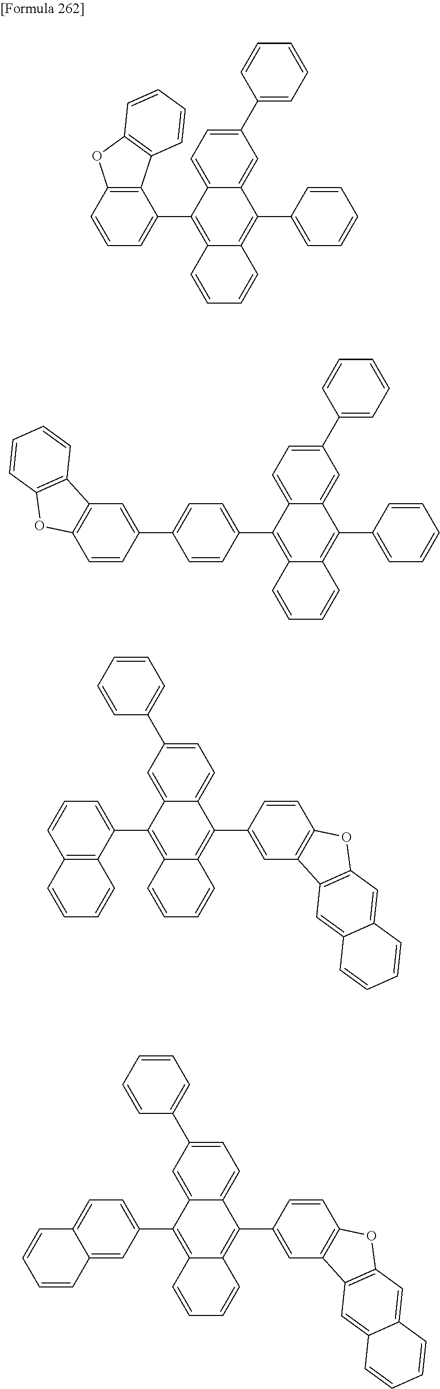 [SMILES: c1ccc(-c2ccc3c(-c4cccc5oc6ccccc6c45)c4ccccc4c(-c4ccccc4)c3c2)cc1 ; c1ccc(-c2ccc3c(-c4ccc(-c5ccc6oc7ccccc7c6c5)cc4)c4ccccc4c(-c4ccccc4)c3c2)cc1 ; c1ccc(-c2ccc3c(-c4ccc5oc6cc7ccccc7cc6c5c4)c4ccccc4c(-c4cccc5ccccc45)c3c2)cc1 ; c1ccc(-c2ccc3c(-c4ccc5oc6cc7ccccc7cc6c5c4)c4ccccc4c(-c4ccc5ccccc5c4)c3c2)cc1]

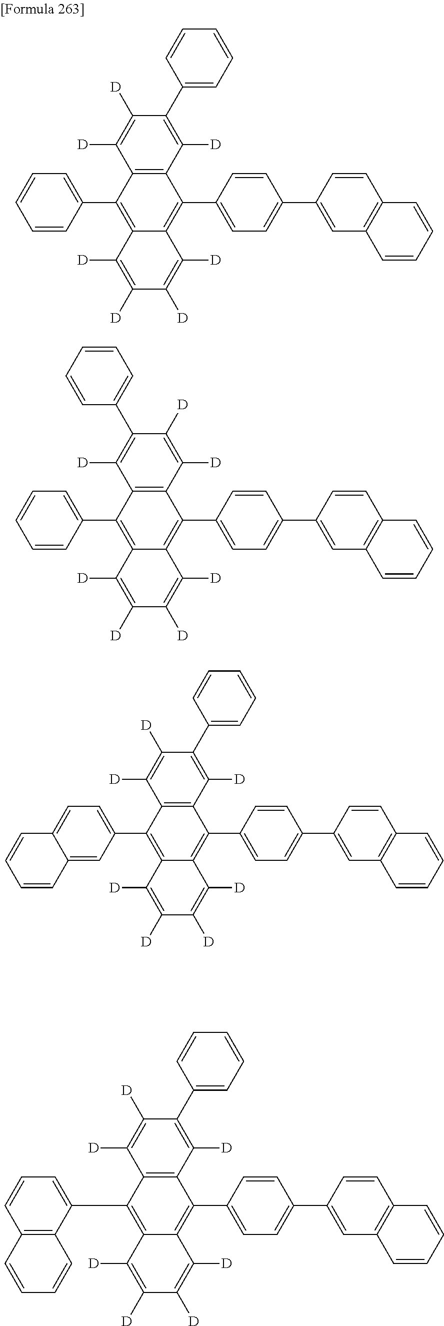

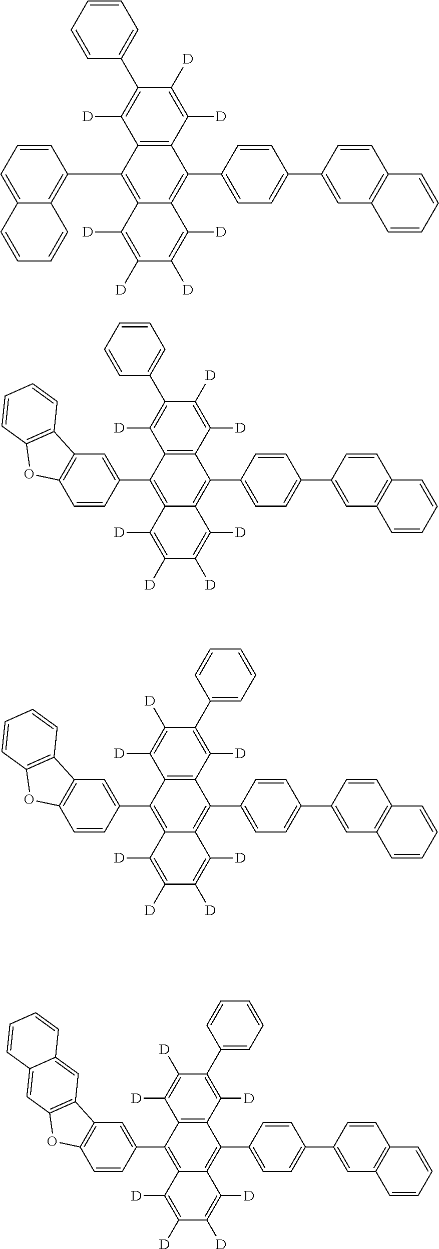

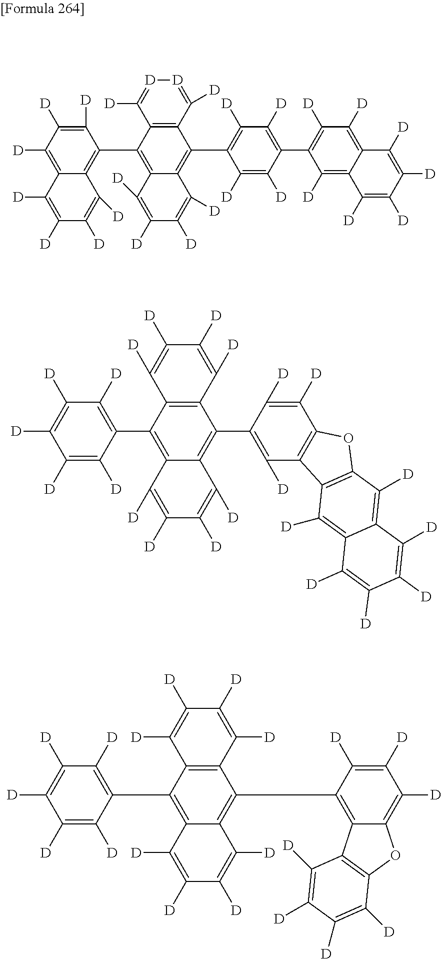

### First Luminescent Compound

In an exemplary arrangement of the organic EL device according to the exemplary embodiment, the first luminescent compound is a compound represented by the formula (6) below.

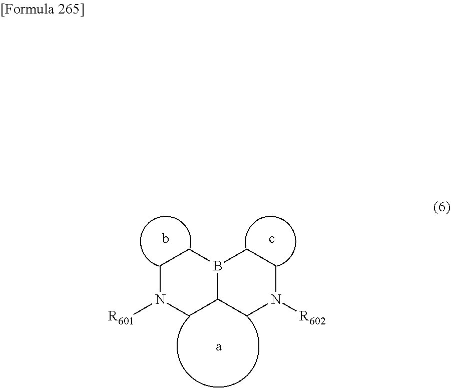

In the formula (6):
a ring a, a ring b and a ring c are each independently a substituted or unsubstituted aromatic hydrocarbon ring having 6 to 50 ring carbon atoms, or a substituted or unsubstituted heterocycle having 5 to 50 ring atoms;
R₆₀₁ and R₆₀₂ are each independently bonded to the ring a, ring b or ring c to form a substituted or unsubstituted heterocycle, or not bonded thereto to form no substituted or unsubstituted heterocycle; and
R₆₀₁ and R₆₀₂ not forming the substituted or unsubstituted heterocycle are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

In an exemplary arrangement of the organic EL device according to the exemplary embodiment, the ring a, ring b and ring c are each a ring (a substituted or unsubstituted aromatic hydrocarbon ring having 6 to 50 ring carbon atoms, or a substituted or unsubstituted heterocycle having 5 to 50 ring atoms) fused with a fused bicyclic structure formed of a boron atom and two nitrogen atoms at the center of the formula (6).

The “aromatic hydrocarbon ring” for the rings a, b, and c has the same structure as a compound formed by introducing a hydrogen atom to the “aryl group”.

Ring atoms of the “aromatic hydrocarbon ring” for the ring a include three carbon atoms on the fused bicyclic structure at the center of the formula (6).

Ring atoms of the “aromatic hydrocarbon ring” for the rings b and c include two carbon atoms on the fused bicyclic structure at the center of the formula (6).

Specific examples of the “substituted or unsubstituted aromatic hydrocarbon ring having 6 to 50 ring carbon atoms” include a compound formed by introducing a hydrogen atom to the “aryl group” described in the specific example group G1.

The “heterocycle” for the rings a, b, and c has the same structure as a compound formed by introducing a hydrogen atom to the “heterocyclic group” described above.

Ring atoms of the “heterocycle” for the ring a include three carbon atoms on the fused bicyclic structure at the center of the formula (6). Ring atoms of the “heterocycle” for the rings b and c include two carbon atoms on the fused bicyclic structure at the center of the formula (6). Specific examples of the “substituted or unsubstituted heterocycle having 5 to 50 ring atoms” include a compound formed by introducing a hydrogen atom to the “heterocyclic group” described in the specific example group G2.

R₆₀₁ and R₆₀₂ may each independently be bonded with the ring a, ring b, or ring c to form a substituted or unsubstituted heterocycle. The “heterocycle” in this arrangement includes a nitrogen atom on the fused bicyclic structure at the center of the formula (6). The heterocycle in the above arrangement optionally includes a hetero atom other than the nitrogen atom. R₆₀₁ and R₆₀₂ being bonded with the ring a, ring b, or ring c specifically means that atoms forming R₆₀₁ and R₆₀₂ are bonded with atoms forming the ring a, ring b, or ring c. For instance, R₆₀₁ may be bonded with the ring a to form a bicyclic (or tri-or-more cyclic) fused nitrogen-containing heterocycle, in which the ring including R₆₀₁ and the ring a are fused. Specific examples of the nitrogen-containing heterocycle include a compound corresponding to the nitrogen-containing bi(or-more)cyclic fused heterocyclic group in the specific example group G2.

The same applies to R₆₀₁ bonded with the ring b, R₆₀₂ bonded with the ring a, and R₆₀₂ bonded with the ring c.

Optionally, R₆₀₁ and R₆₀₂ are each independently not bonded with the ring a, ring b, or ring c.

In an exemplary embodiment, the ring a, ring b and ring c in the formula (6) are each independently a substituted or unsubstituted aromatic hydrocarbon ring having 6 to 50 ring carbon atoms.

In an exemplary embodiment, the ring a, ring b and ring c in the formula (6) are each independently a substituted or unsubstituted benzene ring or a substituted or unsubstituted naphthalene ring.

In an exemplary embodiment, R₆₀₁ and R₆₀₂ in the formula (6) are each independently a substituted or unsubstituted aryl group having 6 to 50 ring carbon atoms, or a substituted or unsubstituted heterocyclic group having 5 to 50 ring atoms, preferably a substituted or unsubstituted aryl group having 6 to 50 ring carbon atoms.

In an exemplary embodiment, the compound represented by the formula (6) is a compound represented by a formula (62) below.

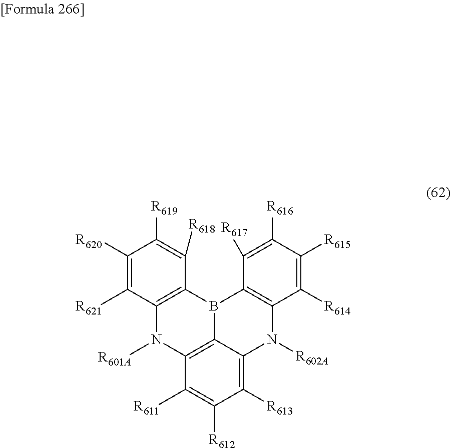

In the formula (62):
R_{601A} is bonded with at least one of R₆₁₁ or R₆₂₁ to form a substituted or unsubstituted heterocycle, or not bonded therewith to form no substituted or unsubstituted heterocycle;
R_{602A} is bonded with at least one of R₆₁₃ or R₆₁₄ to form a substituted or unsubstituted heterocycle, or not bonded therewith to form no substituted or unsubstituted heterocycle;
R_{601A} and R_{602A} not forming the substituted or unsubstituted heterocycle are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
at least one combination of adjacent two or more of R₆₁₁ to R₆₂₁ are mutually bonded to form a substituted or unsubstituted monocyclic ring, mutually bonded to form a substituted or unsubstituted fused ring, or not mutually bonded; and
R₆₁₁ to R₆₂₁ not forming the substituted or unsubstituted heterocycle, not forming the monocyclic ring, and not forming the fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In the formula (62), R₉₀₁, R₉₀₂, R₉₀₃, R₉₀₄, R₉₀₅, R₉₀₆, and R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different; and
when a plurality of R₉₀₇ are present, the plurality of R₉₀₇ are mutually the same or different.

R_{601A} and R_{602A} in the formula (62) are groups corresponding to R₆₀₁ and R₆₀₂ in the formula (6), respectively.

For instance, R_{601A} and R₆₁₁ are optionally bonded with each other to form a bicyclic (or tri-or-more cyclic) fused nitrogen-containing heterocycle, in which the ring including R_{601A} and R₆₁₁ and a benzene ring corresponding to the ring a are fused. Specific examples of the nitrogen-containing heterocycle include a compound corresponding to the nitrogen-containing bi(or-more)cyclic fused heterocyclic group in the specific example group G2. The same applies to R_{601A} bonded with R₆₂₁, R_{602A} bonded with R₆₁₃, and R_{602A} bonded with R₆₁₄.

At least one combination of adjacent two or more of R₆₁₁ to R₆₂₁ are optionally mutually bonded to form a substituted or unsubstituted monocyclic ring, or mutually bonded to form a substituted or unsubstituted fused ring.

For instance, R₆₁₁ and R₆₁₂ are optionally mutually bonded to form a structure in which a benzene ring, indole ring, pyrrole ring, benzofuran ring, benzothiophene ring or the like is fused to the six-membered ring bonded with R₆₁₁ and R₆₁₂, the resultant fused ring forming a naphthalene ring, carbazole ring, indole ring, dibenzofuran ring, or dibenzothiophene ring.

In an exemplary embodiment, R₆₁₁ to R₆₂₁ not contributing to ring formation are each independently a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms.

In an exemplary embodiment, R₆₁₁ to R₆₂₁ not contributing to ring formation are each independently a hydrogen atom, a substituted or unsubstituted aryl group having 6 to 50 ring carbon atoms, or a substituted or unsubstituted heterocyclic group having 5 to 50 ring atoms.

In an exemplary embodiment, R₆₁₁ to R₆₂₁ not contributing to ring formation are each independently a hydrogen atom, or a substituted or unsubstituted alkyl group having 1 to 50 carbon atoms.

In an exemplary embodiment, R₆₁₁ to R₆₂₁ not contributing to ring formation are each independently a hydrogen atom, or a substituted or unsubstituted alkyl group having 1 to 50 carbon atoms; and at least one of R₆₁₁ to R₆₂₁ is a substituted or unsubstituted alkyl group having 1 to 50 carbon atoms.

In an exemplary embodiment, the compound represented by the formula (62) is a compound represented by a formula (63) below.

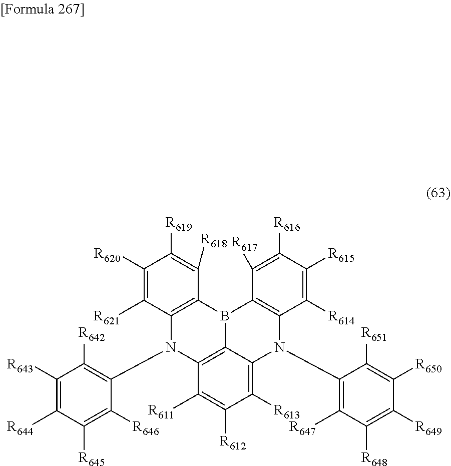

In the formula (63):
R₆₃₁ is bonded with R₆₄₆ to form a substituted or unsubstituted heterocycle, or not bonded therewith to form no substituted or unsubstituted heterocycle;
R₆₃₃ is bonded with R₆₄₇ to form a substituted or unsubstituted heterocycle, or not bonded therewith to form no substituted or unsubstituted heterocycle;
R₆₃₄ is bonded with R₆₅₁ to form a substituted or unsubstituted heterocycle, or not bonded therewith to form no substituted or unsubstituted heterocycle;
R₆₄₁ is bonded with R₆₄₂ to form a substituted or unsubstituted heterocycle, or not bonded therewith to form no substituted or unsubstituted heterocycle;
at least one combination of adjacent two or more of R₆₃₁ to R₆₅₁ are mutually bonded to form a substituted or unsubstituted monocyclic ring, mutually bonded to form a substituted or unsubstituted fused ring, or not mutually bonded; and
R₆₃₁ to R₆₅₁ not forming the substituted or unsubstituted heterocycle, not forming the monocyclic ring, and not forming the fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

R₆₃₁ is optionally bonded with R₆₄₆ to form a substituted or unsubstituted heterocycle. For instance, R₆₃₁ and R₆₄₆ are optionally bonded with each other to form a tri-or-more cyclic fused nitrogen-containing heterocycle, in which a benzene ring bonded with R₆₄₆, a ring including a nitrogen atom, and a benzene ring corresponding to the ring a are fused. Specific examples of the nitrogen-containing heterocycle include a compound corresponding to a nitrogen-containing tri(-or-more)cyclic fused heterocyclic group in the specific example group G2. The same applies to R₆₃₃ bonded with R₆₄₇, R₆₃₄ bonded with R₆₅₁, and R₆₄₁ bonded with R₆₄₂.

In an exemplary embodiment, R₆₃₁ to R₆₅₁ not contributing to ring formation are each independently a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms.

In an exemplary embodiment, R₆₃₁ to R₆₅₁ not contributing to ring formation are each independently a hydrogen atom, a substituted or unsubstituted aryl group having 6 to 50 ring carbon atoms, or a substituted or unsubstituted heterocyclic group having 5 to 50 ring atoms.

In an exemplary embodiment, R₆₃₁ to R₆₅₁ not contributing to ring formation are each independently a hydrogen atom, or a substituted or unsubstituted alkyl group having 1 to 50 carbon atoms.

In an exemplary embodiment, R₆₃₁ to R₆₅₁ not contributing to ring formation are each independently a hydrogen atom, or a substituted or unsubstituted alkyl group having 1 to 50 carbon atoms; and
at least one of R₆₃₁ to R₆₅₁ is a substituted or unsubstituted alkyl group having 1 to 50 carbon atoms.

In an exemplary embodiment, the compound represented by the formula (63) is a compound represented by a formula (63A) below.

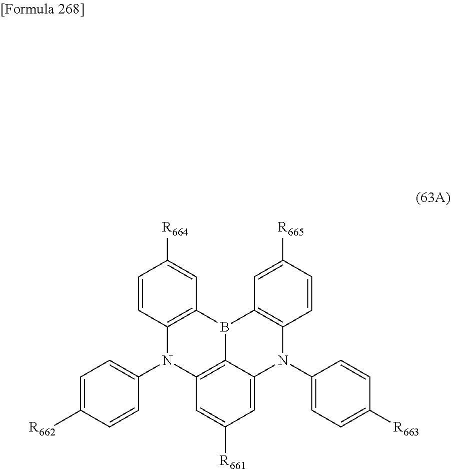

In the formula (63A):
R₆₆₁ is a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, or a substituted or unsubstituted aryl group having 6 to 50 ring carbon atoms, and
R₆₆₂ to R₆₆₅ are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, or a substituted or unsubstituted aryl group having 6 to 50 ring carbon atoms.

In an exemplary embodiment, R₆₆₁ to R₆₆₅ are each independently a substituted or unsubstituted alkyl group having 1 to 50 carbon atoms, or a substituted or unsubstituted aryl group having 6 to 50 ring carbon atoms.

In an exemplary embodiment, R₆₆₁ to R₆₆₅ are each independently a substituted or unsubstituted alkyl group having 1 to 50 carbon atoms.

In an exemplary embodiment, the compound represented by the formula (63) is a compound represented by a formula (63B) below.

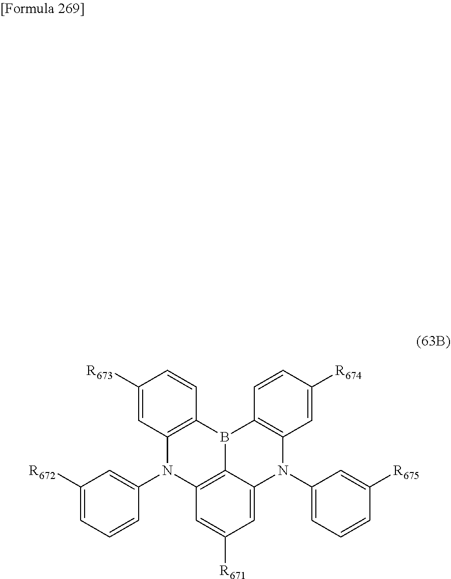

In the formula (63B):
R₆₇₁ and R₆₇₂ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —N(R₉₀₆)(R₉₀₇), or a substituted or unsubstituted aryl group having 6 to 50 ring carbon atoms; and
R₆₇₃ to R₆₇₅ are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —N(R₉₀₆)(R₉₀₇), or a substituted or unsubstituted aryl group having 6 to 50 ring carbon atoms.

In an exemplary embodiment, the compound represented by the formula (63) is a compound represented by a formula (63B′) below.

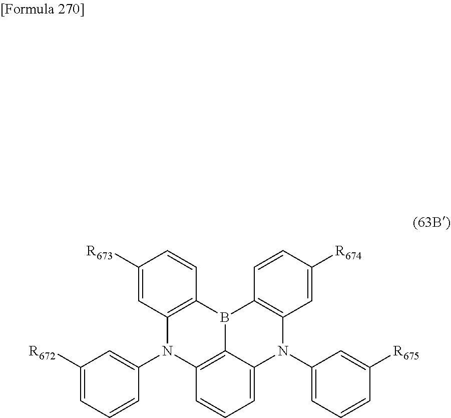

In the formula (63B′), R₆₇₂ to R₆₇₅ each independently represent the same as R₆₇₂ to R₆₇₅ in the formula (63B).

In an exemplary embodiment, at least one of R₆₇₁ to R₆₇₅ is a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —N(R₉₀₆)(R₉₀₇), or a substituted or unsubstituted aryl group having 6 to 50 ring carbon atoms.

In an exemplary embodiment:
R₆₇₂ is a hydrogen atom, a substituted or unsubstituted alkyl group having 1 to 50 carbon atoms, a group represented by —N(R₉₀₆)(R₉₀₇), or a substituted or unsubstituted aryl group having 6 to 50 ring carbon atoms; and
R₆₇₁ and R₆₇₃ to R₆₇₅ are each independently a substituted or unsubstituted alkyl group having 1 to 50 carbon atoms, a group represented by —N(R₉₀₆)(R₉₀₇), or a substituted or unsubstituted aryl group having 6 to 50 ring carbon atoms.

In an exemplary embodiment, the compound represented by the formula (63) is a compound represented by a formula (63C) below.

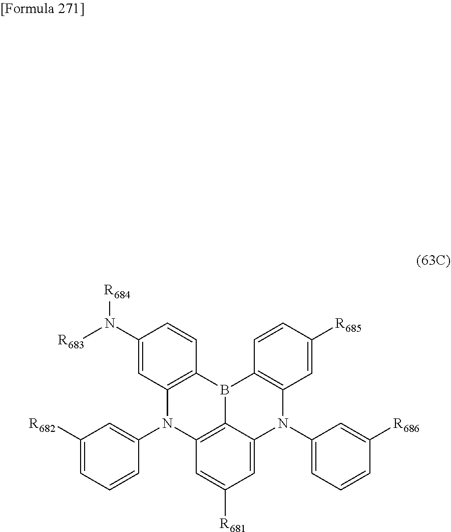

In the formula (63C):
R₆₈₁ and R₆₈₂ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, or a substituted or unsubstituted aryl group having 6 to 50 ring carbon atoms; and
R₆₈₃ to R₆₈₆ are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, or a substituted or unsubstituted aryl group having 6 to 50 ring carbon atoms.

In an exemplary embodiment, the compound represented by the formula (63) is a compound represented by a formula (63C′) below.

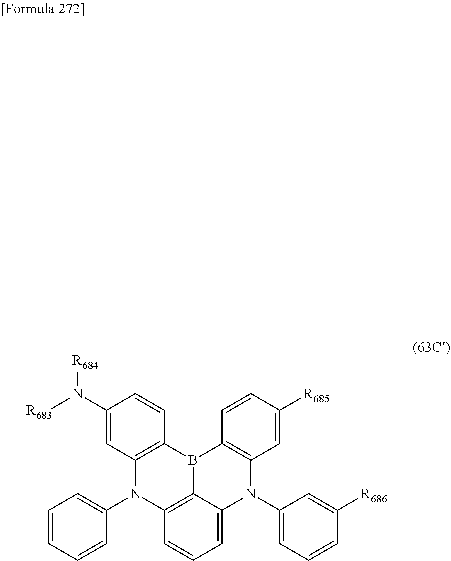

In the formula (63C′), R₆₈₃ to R₆₈₆ each independently represent the same as R₆₈₃ to R₆₈₆ in the formula (63C).

In an exemplary embodiment, R₆₈₁ to R₆₈₆ are each independently a substituted or unsubstituted alkyl group having 1 to 50 carbon atoms, or a substituted or unsubstituted aryl group having 6 to 50 ring carbon atoms.

In an exemplary embodiment, R₆₈₁ to R₆₈₆ are each independently a substituted or unsubstituted aryl group having 6 to 50 carbon atoms.

The compound represented by the formula (6) is producible by initially bonding the ring a, ring b and ring c with linking groups (a group including N—R₆₀₁ and a group including N—R₆₀₂) to form an intermediate (first reaction), and bonding the ring a, ring b and ring c with a linking group (a group including a boron atom) to form a final product (second reaction). In the first reaction, an amination reaction (e.g. Buchwald-Hartwig reaction) is applicable. In the second reaction, Tandem Hetero-Friedel-Crafts Reactions or the like is applicable.

In an exemplary embodiment, the compound represented by the formula (6) is a compound represented by a formula (42-2) below.

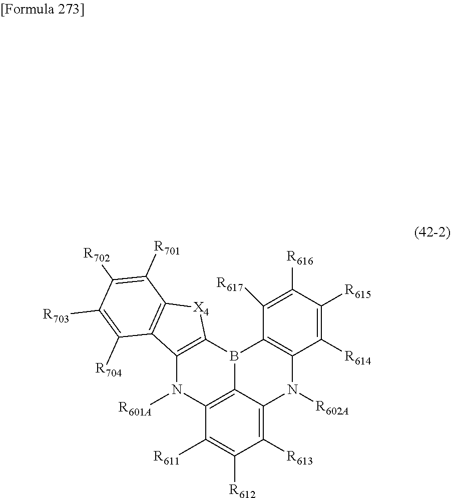

In the formula (42-2), R₆₁₁ to R₆₁₇, R_{601A} and R_{602A} each independently represent the same as R₆₁₁ to R₆₁₇, R_{601A} and R_{602A} in the formula (62);
X₄ is an oxygen atom or a sulfur atom;
at least one combination of adjacent two or more of R₇₀₁ to R₇₀₄ are mutually bonded to form a substituted or unsubstituted monocyclic ring, mutually bonded to form a substituted or unsubstituted fused ring, or not mutually bonded; and
R₇₀₁ to R₇₀₄ forming neither the substituted or unsubstituted monocyclic ring nor the fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In the formula (42-2), R₉₀₁, R₉₀₂, R₉₀₃, R₉₀₄, R₉₀₅, R₉₀₆ and R₉₀₇ each independently represent the same as R₉₀₁, R₉₀₂, R₉₀₃, R₉₀₄, R₉₀₅, R₉₀₆ and R₉₀₇ in the formula (62).

### Specific Examples of Compound Represented by Formula (6)

Specific examples of the compound represented by the formula (6) are given below. It should however be noted that these specific examples are merely exemplary and do not limit the compound represented by the formula (6).

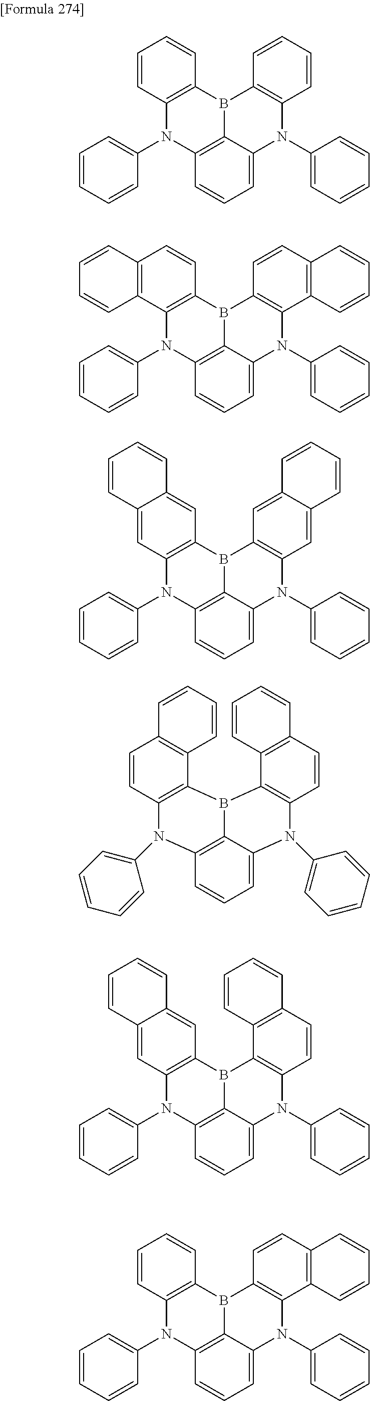

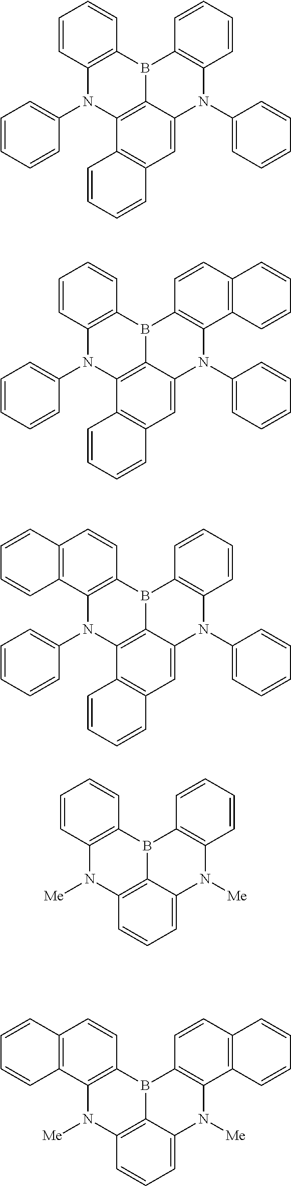

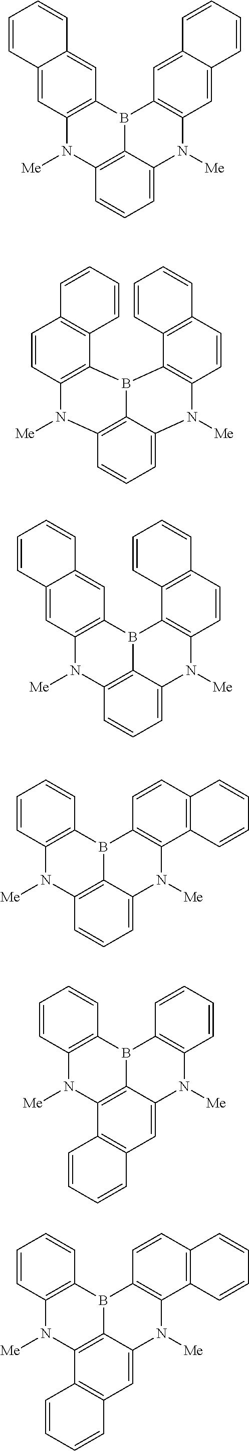 [SMILES: CN1c2cc3ccccc3cc2B2c3cc4ccccc4cc3N(C)c3cccc1c32 ; CN1c2cccc3c2B(c2c1ccc1ccccc21)c1c(ccc2ccccc12)N3C ; CN1c2cc3ccccc3cc2B2c3c1cccc3N(C)c1ccc3ccccc3c12 ; CN1c2ccccc2B2c3ccc4ccccc4c3N(C)c3cccc1c32 ; CN1c2ccccc2B2c3ccccc3N(C)c3c2c1cc1ccccc31 ; CN1c2cc3ccccc3c3c2B(c2ccccc2N3C)c2ccc3ccccc3c21]

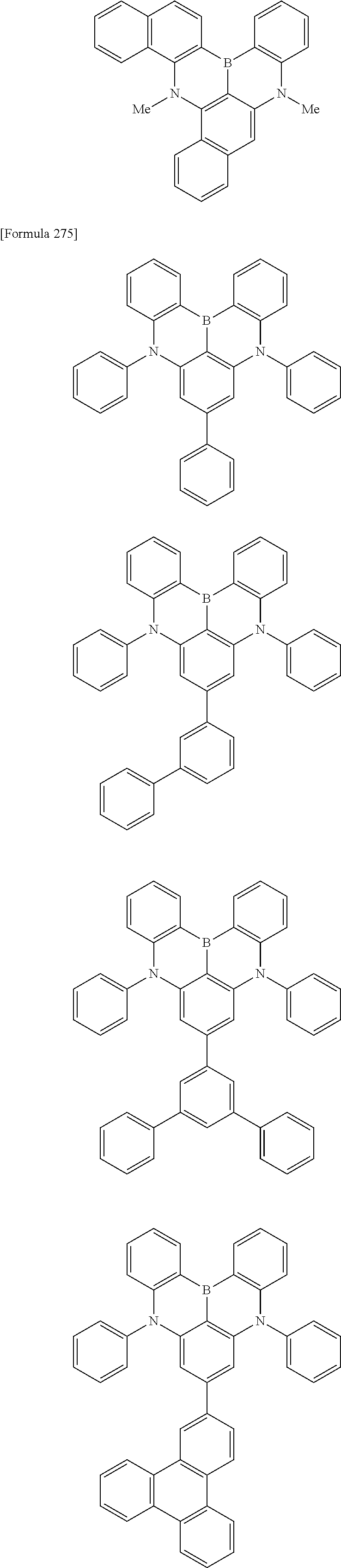 [SMILES: CN1c2ccccc2B2c3ccc4ccccc4c3N(C)c3c2c1cc1ccccc31 ; c1ccc(-c2cc3c4c(c2)N(c2ccccc2)c2ccccc2B4c2ccccc2N3c2ccccc2)cc1 ; c1ccc(-c2cccc(-c3cc4c5c(c3)N(c3ccccc3)c3ccccc3B5c3ccccc3N4c3ccccc3)c2)cc1 ; c1ccc(-c2cc(-c3ccccc3)cc(-c3cc4c5c(c3)N(c3ccccc3)c3ccccc3B5c3ccccc3N4c3ccccc3)c2)cc1 ; c1ccc(N2c3ccccc3B3c4ccccc4N(c4ccccc4)c4cc(-c5ccc6c7ccccc7c7ccccc7c6c5)cc2c43)cc1]

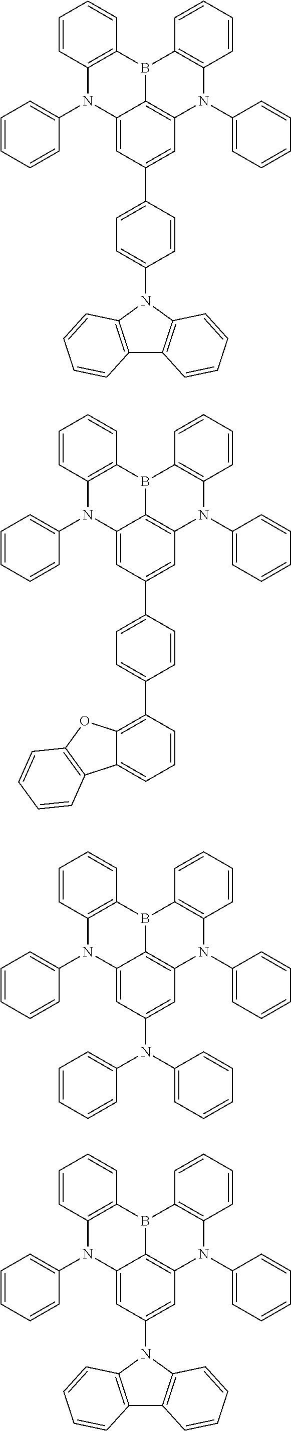

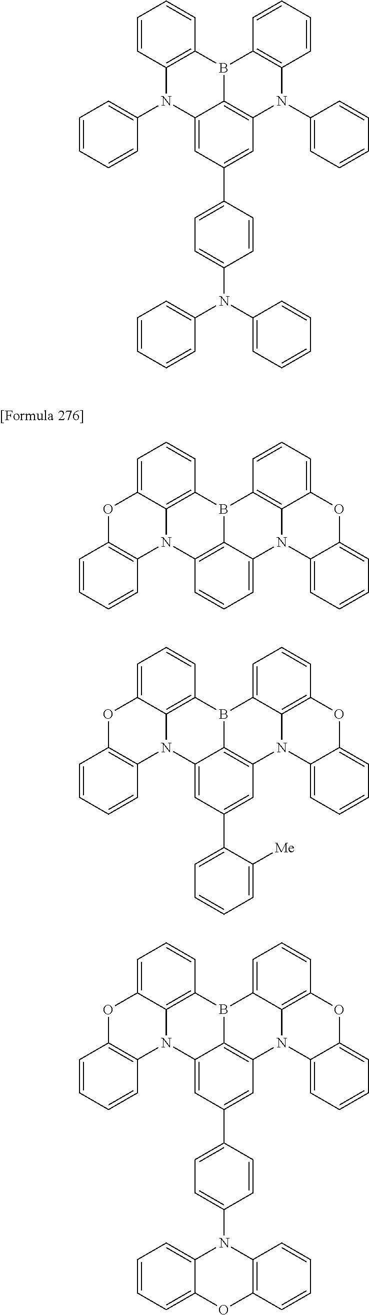 [SMILES: c1ccc(N(c2ccccc2)c2ccc(-c3cc4c5c(c3)N(c3ccccc3)c3ccccc3B5c3ccccc3N4c3ccccc3)cc2)cc1 ; c1ccc2c(c1)Oc1cccc3c1N2c1cccc2c1B3c1cccc3c1N2c1ccccc1O3 ; Cc1ccccc1-c1cc2c3c(c1)N1c4ccccc4Oc4cccc(c41)B3c1cccc3c1N2c1ccccc1O3 ; c1ccc2c(c1)Oc1ccccc1N2c1ccc(-c2cc3c4c(c2)N2c5ccccc5Oc5cccc(c52)B4c2cccc4c2N3c2ccccc2O4)cc1]

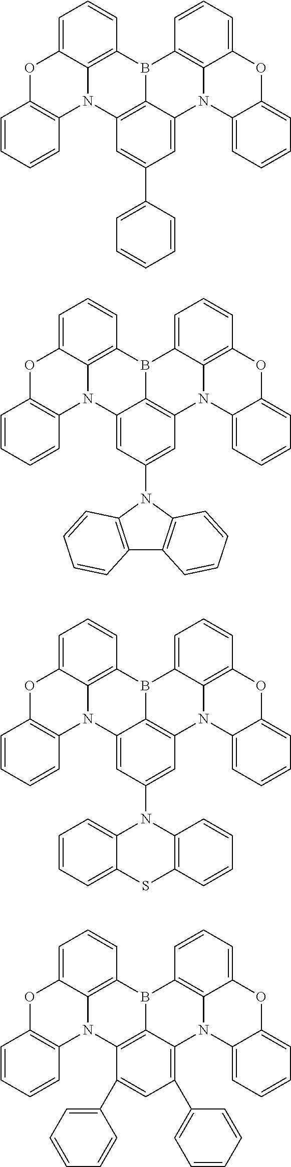

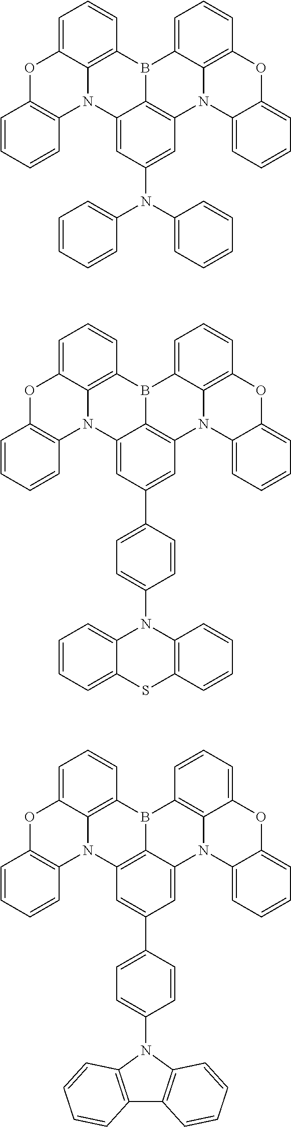 [SMILES: c1ccc(N(c2ccccc2)c2cc3c4c(c2)N2c5ccccc5Oc5cccc(c52)B4c2cccc4c2N3c2ccccc2O4)cc1 ; c1ccc2c(c1)Oc1cccc3c1N2c1cc(-c2ccc(N4c5ccccc5Sc5ccccc54)cc2)cc2c1B3c1cccc3c1N2c1ccccc1O3 ; c1ccc2c(c1)Oc1cccc3c1N2c1cc(-c2ccc(-n4c5ccccc5c5ccccc54)cc2)cc2c1B3c1cccc3c1N2c1ccccc1O3]

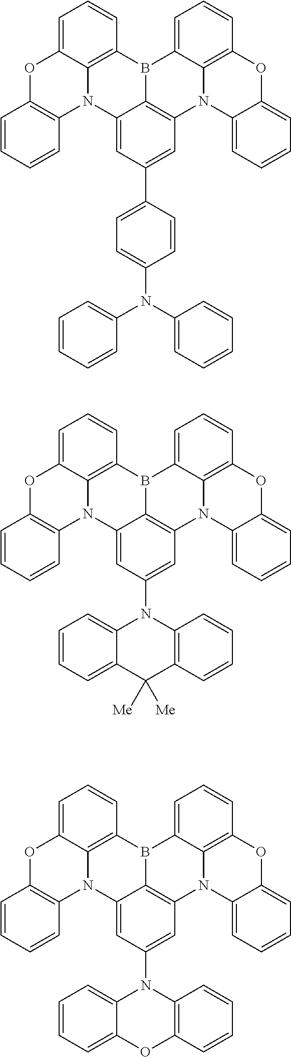

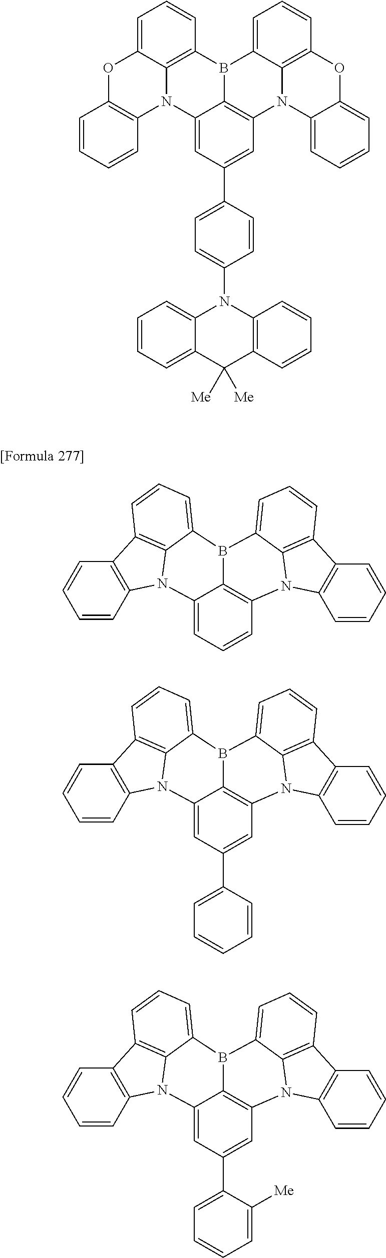 [SMILES: CC1(C)c2ccccc2N(c2ccc(-c3cc4c5c(c3)N3c6ccccc6Oc6cccc(c63)B5c3cccc5c3N4c3ccccc3O5)cc2)c2ccccc21 ; c1cc2c3c(c1)-n1c4ccccc4c4cccc(c41)B3c1cccc3c4ccccc4n-2c13 ; c1ccc(-c2cc3c4c(c2)-n2c5ccccc5c5cccc(c52)B4c2cccc4c5ccccc5n-3c24)cc1 ; Cc1ccccc1-c1cc2c3c(c1)-n1c4ccccc4c4cccc(c41)B3c1cccc3c4ccccc4n-2c13]

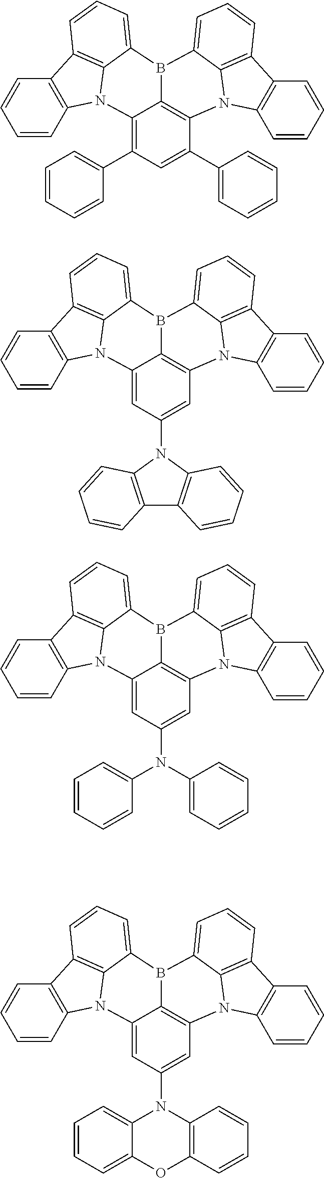

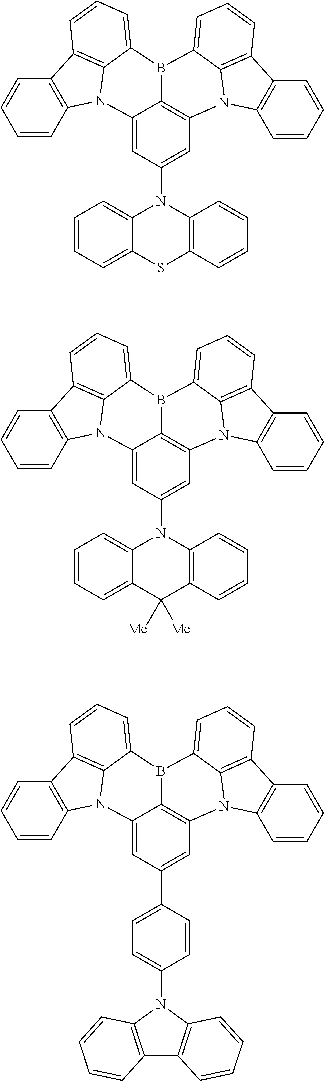 [SMILES: c1ccc2c(c1)Sc1ccccc1N2c1cc2c3c(c1)-n1c4ccccc4c4cccc(c41)B3c1cccc3c4ccccc4n-2c13 ; CC1(C)c2ccccc2N(c2cc3c4c(c2)-n2c5ccccc5c5cccc(c52)B4c2cccc4c5ccccc5n-3c24)c2ccccc21 ; c1ccc2c(c1)c1ccccc1n2-c1ccc(-c2cc3c4c(c2)-n2c5ccccc5c5cccc(c52)B4c2cccc4c5ccccc5n-3c24)cc1]

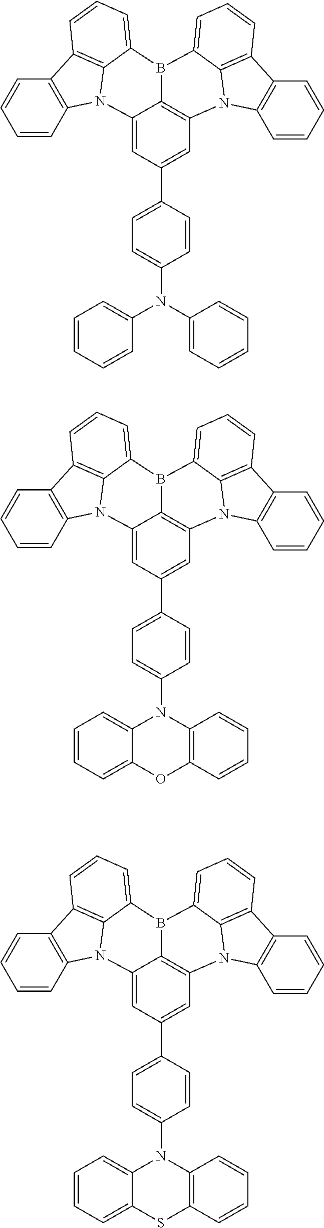

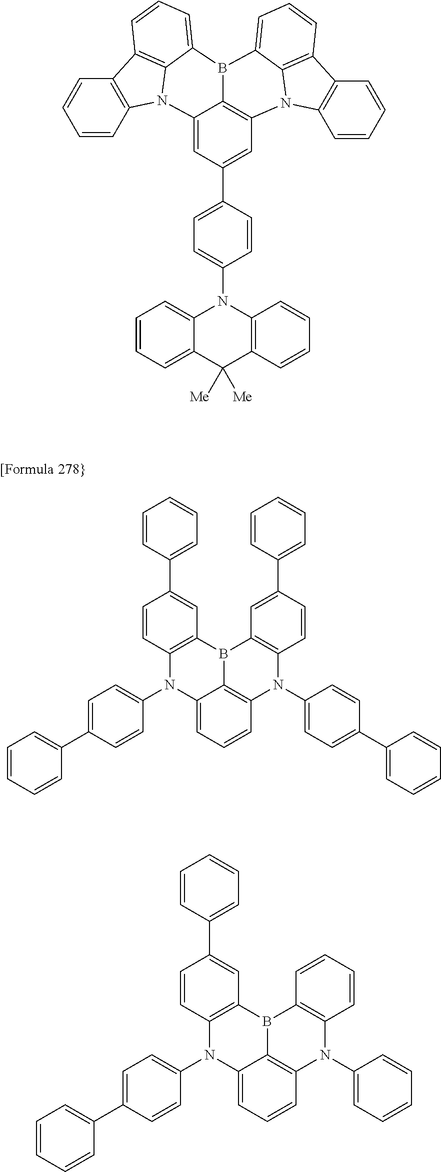 [SMILES: CC1(C)c2ccccc2N(c2ccc(-c3cc4c5c(c3)-n3c6ccccc6c6cccc(c63)B5c3cccc5c6ccccc6n-4c35)cc2)c2ccccc21 ; c1ccc(-c2ccc(N3c4ccc(-c5ccccc5)cc4B4c5cc(-c6ccccc6)ccc5N(c5ccc(-c6ccccc6)cc5)c5cccc3c54)cc2)cc1 ; c1ccc(-c2ccc(N3c4ccc(-c5ccccc5)cc4B4c5ccccc5N(c5ccccc5)c5cccc3c54)cc2)cc1]

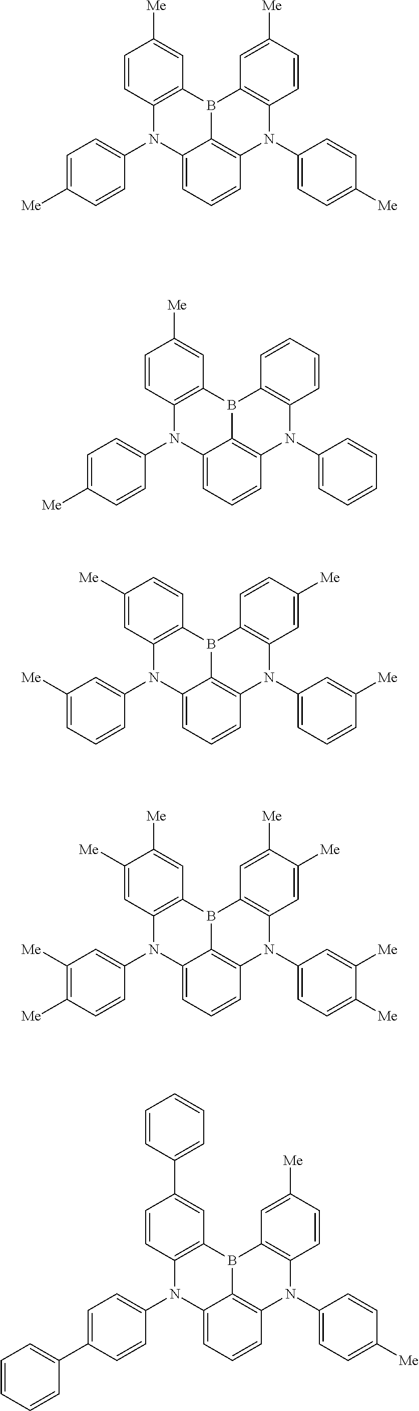 [SMILES: Cc1ccc(N2c3ccc(C)cc3B3c4cc(C)ccc4N(c4ccc(C)cc4)c4cccc2c43)cc1 ; Cc1ccc(N2c3ccc(C)cc3B3c4ccccc4N(c4ccccc4)c4cccc2c43)cc1 ; Cc1cccc(N2c3cc(C)ccc3B3c4ccc(C)cc4N(c4cccc(C)c4)c4cccc2c43)c1 ; Cc1ccc(N2c3cc(C)c(C)cc3B3c4cc(C)c(C)cc4N(c4ccc(C)c(C)c4)c4cccc2c43)cc1C ; Cc1ccc(N2c3ccc(C)cc3B3c4cc(-c5ccccc5)ccc4N(c4ccc(-c5ccccc5)cc4)c4cccc2c43)cc1]

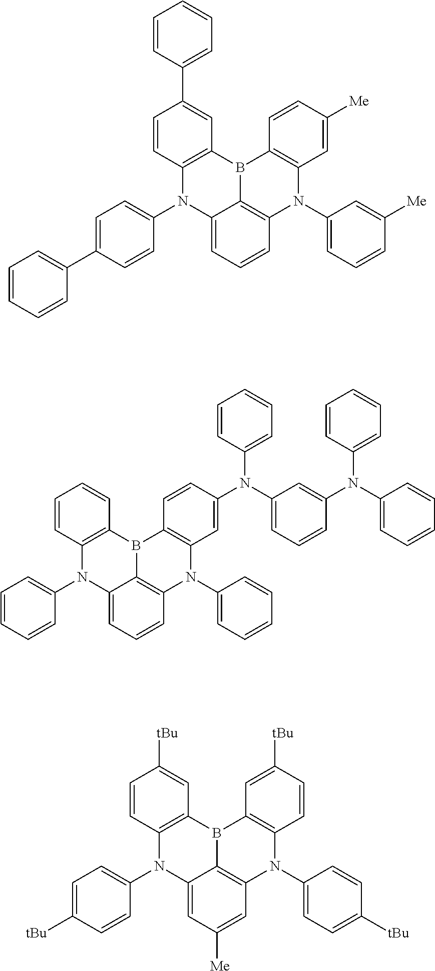 [SMILES: Cc1cccc(N2c3cc(C)ccc3B3c4cc(-c5ccccc5)ccc4N(c4ccc(-c5ccccc5)cc4)c4cccc2c43)c1 ; c1ccc(N(c2ccccc2)c2cccc(N(c3ccccc3)c3ccc4c(c3)N(c3ccccc3)c3cccc5c3B4c3ccccc3N5c3ccccc3)c2)cc1 ; Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1]

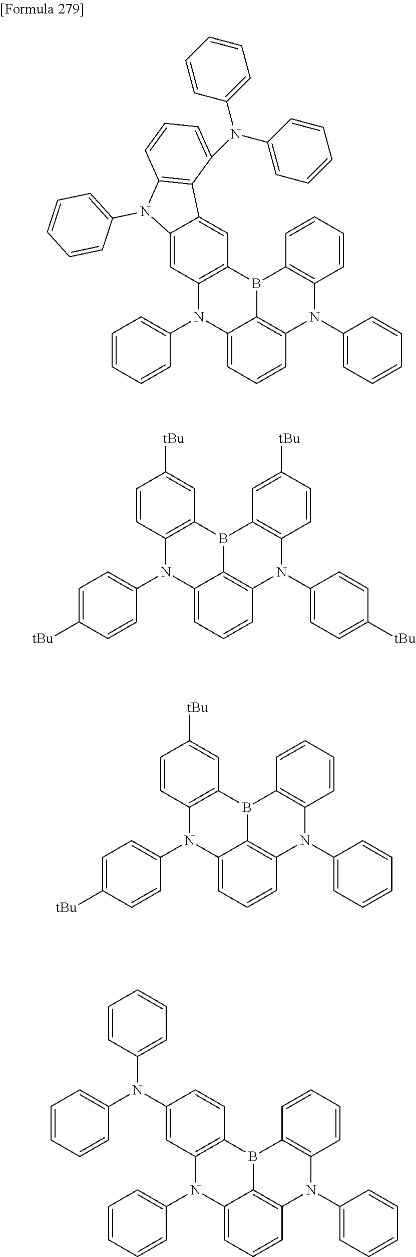 [SMILES: c1ccc(N2c3ccccc3B3c4cc5c6c(N(c7ccccc7)c7ccccc7)cccc6n(-c6ccccc6)c5cc4N(c4ccccc4)c4cccc2c43)cc1 ; CC(C)(C)c1ccc(N2c3ccc(C(C)(C)C)cc3B3c4cc(C(C)(C)C)ccc4N(c4ccc(C(C)(C)C)cc4)c4cccc2c43)cc1 ; CC(C)(C)c1ccc(N2c3ccc(C(C)(C)C)cc3B3c4ccccc4N(c4ccccc4)c4cccc2c43)cc1 ; c1ccc(N(c2ccccc2)c2ccc3c(c2)N(c2ccccc2)c2cccc4c2B3c2ccccc2N4c2ccccc2)cc1]

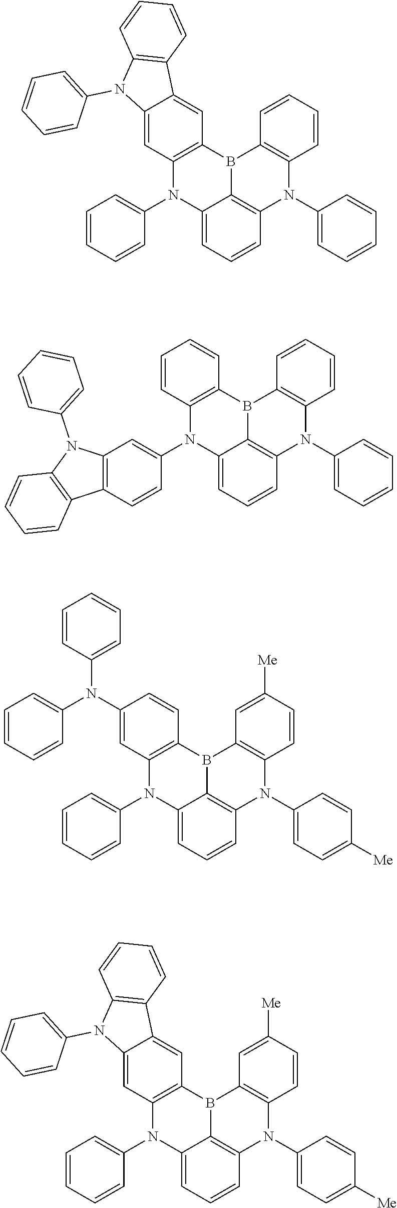 [SMILES: c1ccc(N2c3ccccc3B3c4cc5c6ccccc6n(-c6ccccc6)c5cc4N(c4ccccc4)c4cccc2c43)cc1 ; c1ccc(N2c3ccccc3B3c4ccccc4N(c4ccc5c6ccccc6n(-c6ccccc6)c5c4)c4cccc2c43)cc1 ; Cc1ccc(N2c3ccc(C)cc3B3c4ccc(N(c5ccccc5)c5ccccc5)cc4N(c4ccccc4)c4cccc2c43)cc1 ; Cc1ccc(N2c3ccc(C)cc3B3c4cc5c6ccccc6n(-c6ccccc6)c5cc4N(c4ccccc4)c4cccc2c43)cc1]

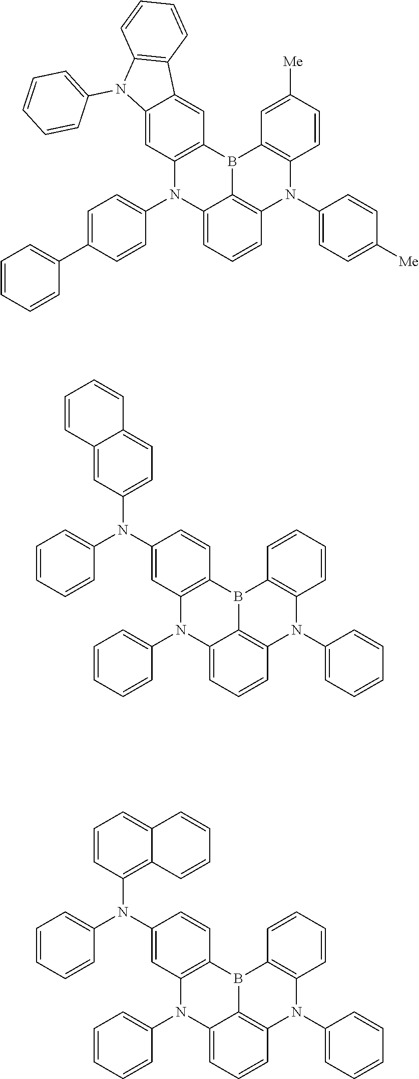 [SMILES: Cc1ccc(N2c3ccc(C)cc3B3c4cc5c6ccccc6n(-c6ccccc6)c5cc4N(c4ccc(-c5ccccc5)cc4)c4cccc2c43)cc1 ; c1ccc(N(c2ccc3c(c2)N(c2ccccc2)c2cccc4c2B3c2ccccc2N4c2ccccc2)c2ccc3ccccc3c2)cc1 ; c1ccc(N2c3ccccc3B3c4ccc(N(c5ccccc5)c5cccc6ccccc56)cc4N(c4ccccc4)c4cccc2c43)cc1]

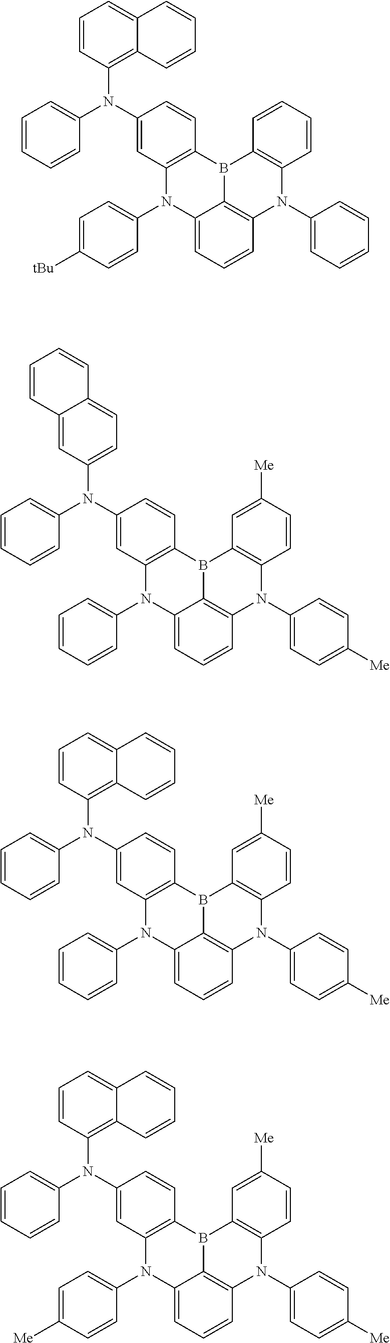 [SMILES: CC(C)(C)c1ccc(N2c3cc(N(c4ccccc4)c4cccc5ccccc45)ccc3B3c4ccccc4N(c4ccccc4)c4cccc2c43)cc1 ; Cc1ccc(N2c3ccc(C)cc3B3c4ccc(N(c5ccccc5)c5ccc6ccccc6c5)cc4N(c4ccccc4)c4cccc2c43)cc1 ; Cc1ccc(N2c3ccc(C)cc3B3c4ccc(N(c5ccccc5)c5cccc6ccccc56)cc4N(c4ccccc4)c4cccc2c43)cc1 ; Cc1ccc(N2c3ccc(C)cc3B3c4ccc(N(c5ccccc5)c5cccc6ccccc56)cc4N(c4ccc(C)cc4)c4cccc2c43)cc1]

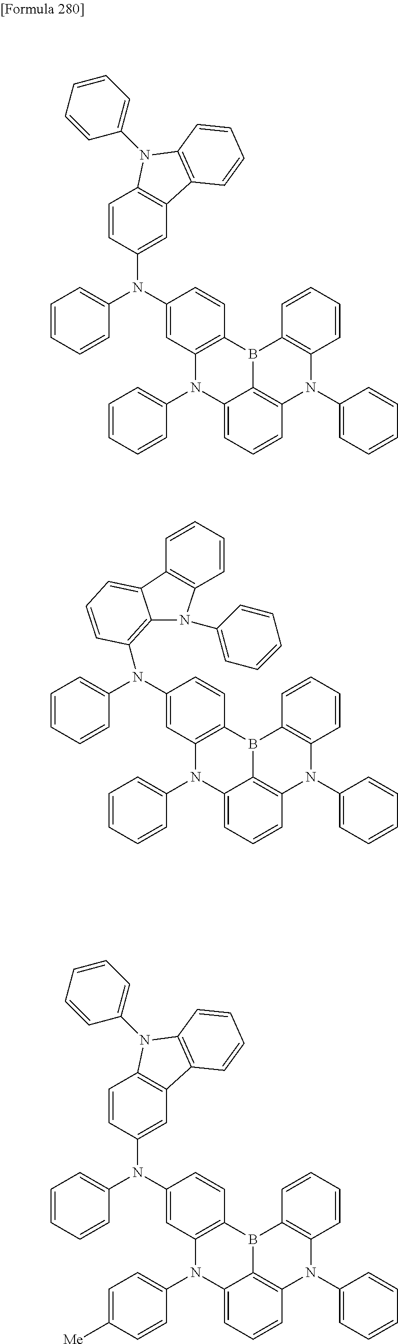 [SMILES: c1ccc(N(c2ccc3c(c2)N(c2ccccc2)c2cccc4c2B3c2ccccc2N4c2ccccc2)c2ccc3c(c2)c2ccccc2n3-c2ccccc2)cc1 ; c1ccc(N2c3ccccc3B3c4ccc(N(c5ccccc5)c5cccc6c7ccccc7n(-c7ccccc7)c56)cc4N(c4ccccc4)c4cccc2c43)cc1 ; Cc1ccc(N2c3cc(N(c4ccccc4)c4ccc5c(c4)c4ccccc4n5-c4ccccc4)ccc3B3c4ccccc4N(c4ccccc4)c4cccc2c43)cc1]

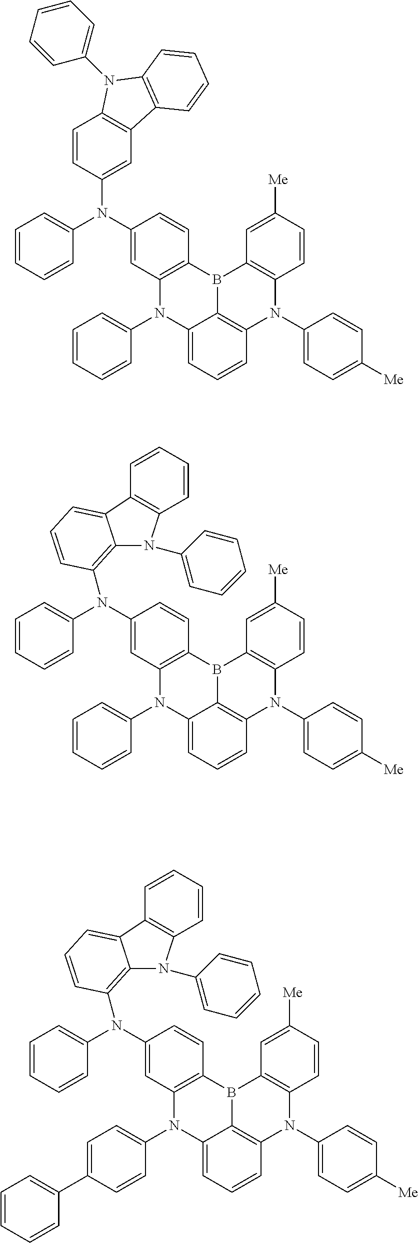 [SMILES: Cc1ccc(N2c3ccc(C)cc3B3c4ccc(N(c5ccccc5)c5ccc6c(c5)c5ccccc5n6-c5ccccc5)cc4N(c4ccccc4)c4cccc2c43)cc1 ; Cc1ccc(N2c3ccc(C)cc3B3c4ccc(N(c5ccccc5)c5cccc6c7ccccc7n(-c7ccccc7)c56)cc4N(c4ccccc4)c4cccc2c43)cc1 ; Cc1ccc(N2c3ccc(C)cc3B3c4ccc(N(c5ccccc5)c5cccc6c7ccccc7n(-c7ccccc7)c56)cc4N(c4ccc(-c5ccccc5)cc4)c4cccc2c43)cc1]

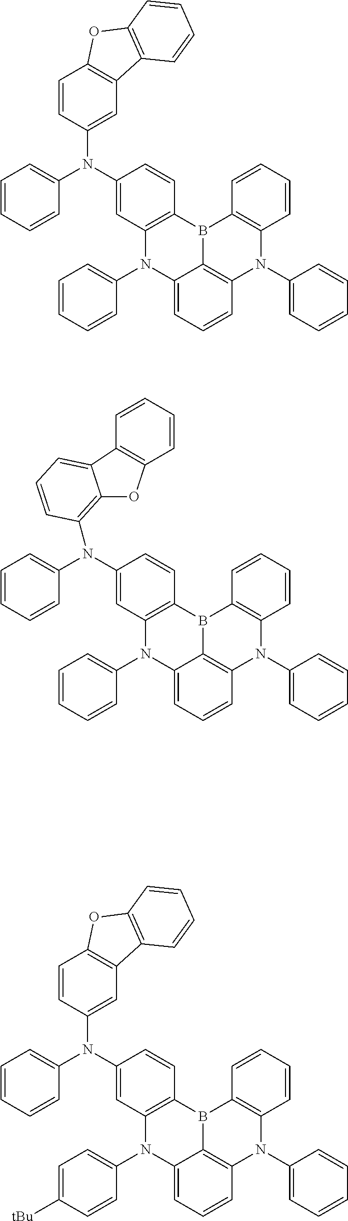 [SMILES: c1ccc(N(c2ccc3c(c2)N(c2ccccc2)c2cccc4c2B3c2ccccc2N4c2ccccc2)c2ccc3oc4ccccc4c3c2)cc1 ; c1ccc(N2c3ccccc3B3c4ccc(N(c5ccccc5)c5cccc6c5oc5ccccc56)cc4N(c4ccccc4)c4cccc2c43)cc1 ; CC(C)(C)c1ccc(N2c3cc(N(c4ccccc4)c4ccc5oc6ccccc6c5c4)ccc3B3c4ccccc4N(c4ccccc4)c4cccc2c43)cc1]

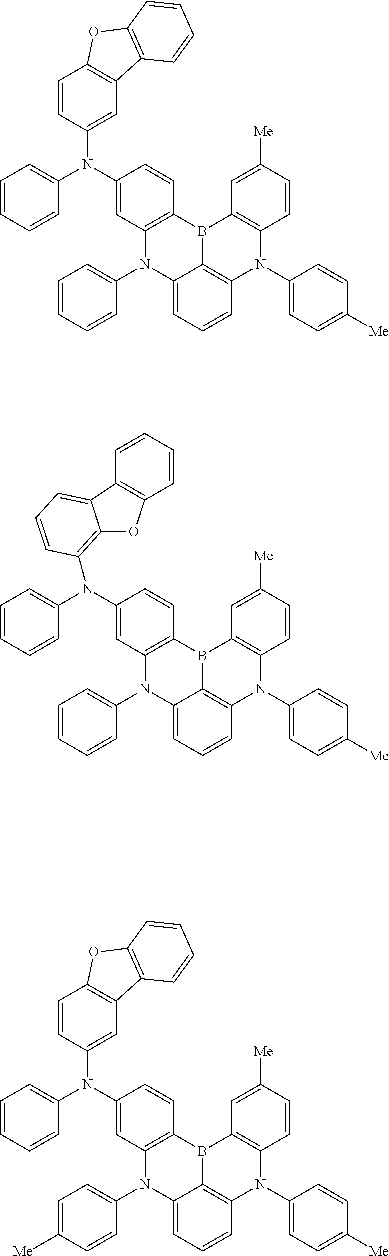 [SMILES: Cc1ccc(N2c3ccc(C)cc3B3c4ccc(N(c5ccccc5)c5ccc6oc7ccccc7c6c5)cc4N(c4ccccc4)c4cccc2c43)cc1 ; Cc1ccc(N2c3ccc(C)cc3B3c4ccc(N(c5ccccc5)c5cccc6c5oc5ccccc56)cc4N(c4ccccc4)c4cccc2c43)cc1 ; Cc1ccc(N2c3ccc(C)cc3B3c4ccc(N(c5ccccc5)c5ccc6oc7ccccc7c6c5)cc4N(c4ccc(C)cc4)c4cccc2c43)cc1]

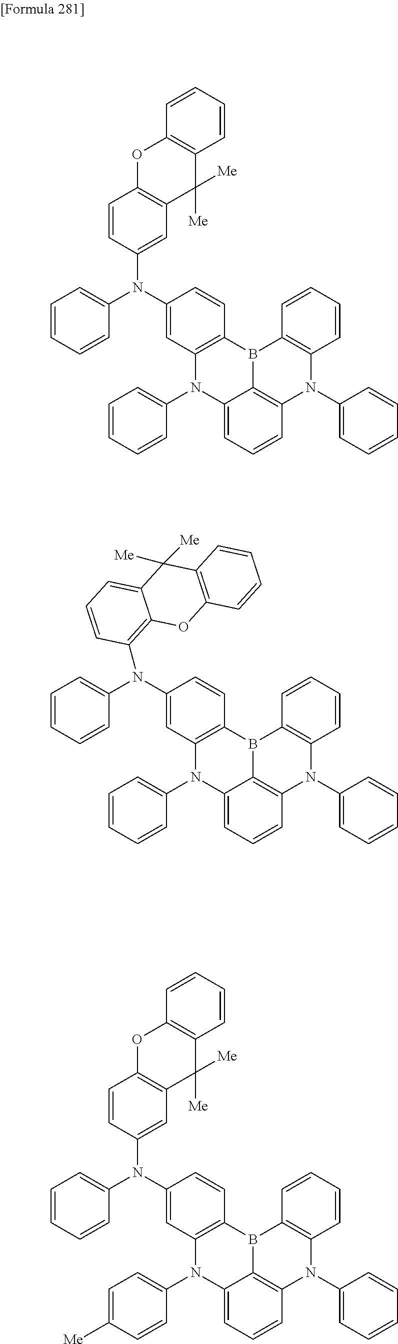 [SMILES: CC1(C)c2ccccc2Oc2ccc(N(c3ccccc3)c3ccc4c(c3)N(c3ccccc3)c3cccc5c3B4c3ccccc3N5c3ccccc3)cc21 ; CC1(C)c2ccccc2Oc2c(N(c3ccccc3)c3ccc4c(c3)N(c3ccccc3)c3cccc5c3B4c3ccccc3N5c3ccccc3)cccc21 ; Cc1ccc(N2c3cc(N(c4ccccc4)c4ccc5c(c4)C(C)(C)c4ccccc4O5)ccc3B3c4ccccc4N(c4ccccc4)c4cccc2c43)cc1]

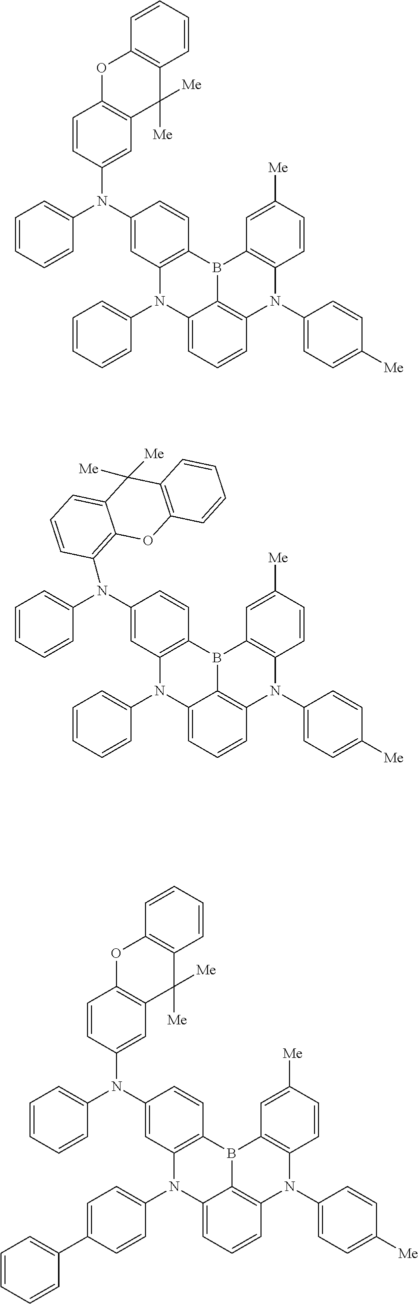 [SMILES: Cc1ccc(N2c3ccc(C)cc3B3c4ccc(N(c5ccccc5)c5ccc6c(c5)C(C)(C)c5ccccc5O6)cc4N(c4ccccc4)c4cccc2c43)cc1 ; Cc1ccc(N2c3ccc(C)cc3B3c4ccc(N(c5ccccc5)c5cccc6c5Oc5ccccc5C6(C)C)cc4N(c4ccccc4)c4cccc2c43)cc1 ; Cc1ccc(N2c3ccc(C)cc3B3c4ccc(N(c5ccccc5)c5ccc6c(c5)C(C)(C)c5ccccc5O6)cc4N(c4ccc(-c5ccccc5)cc4)c4cccc2c43)cc1]

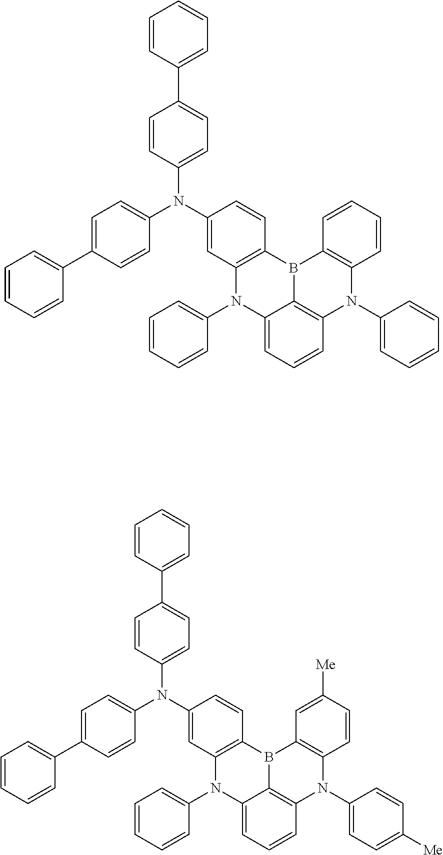 [SMILES: c1ccc(-c2ccc(N(c3ccc(-c4ccccc4)cc3)c3ccc4c(c3)N(c3ccccc3)c3cccc5c3B4c3ccccc3N5c3ccccc3)cc2)cc1 ; Cc1ccc(N2c3ccc(C)cc3B3c4ccc(N(c5ccc(-c6ccccc6)cc5)c5ccc(-c6ccccc6)cc5)cc4N(c4ccccc4)c4cccc2c43)cc1]

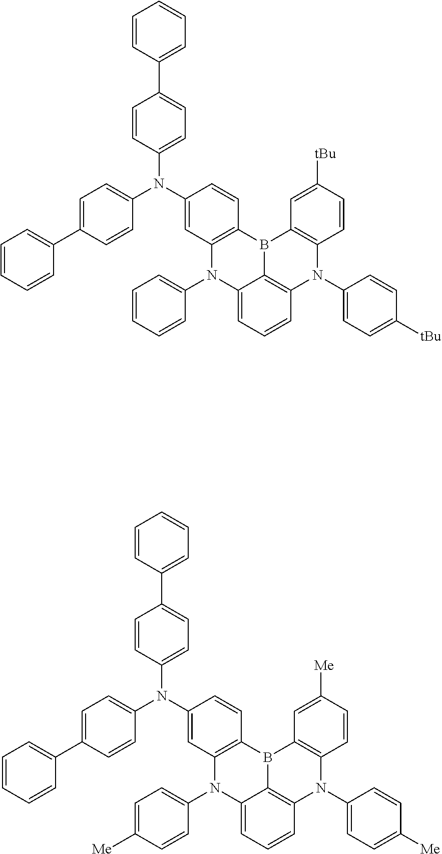 [SMILES: CC(C)(C)c1ccc(N2c3ccc(C(C)(C)C)cc3B3c4ccc(N(c5ccc(-c6ccccc6)cc5)c5ccc(-c6ccccc6)cc5)cc4N(c4ccccc4)c4cccc2c43)cc1 ; Cc1ccc(N2c3ccc(C)cc3B3c4ccc(N(c5ccc(-c6ccccc6)cc5)c5ccc(-c6ccccc6)cc5)cc4N(c4ccc(C)cc4)c4cccc2c43)cc1]

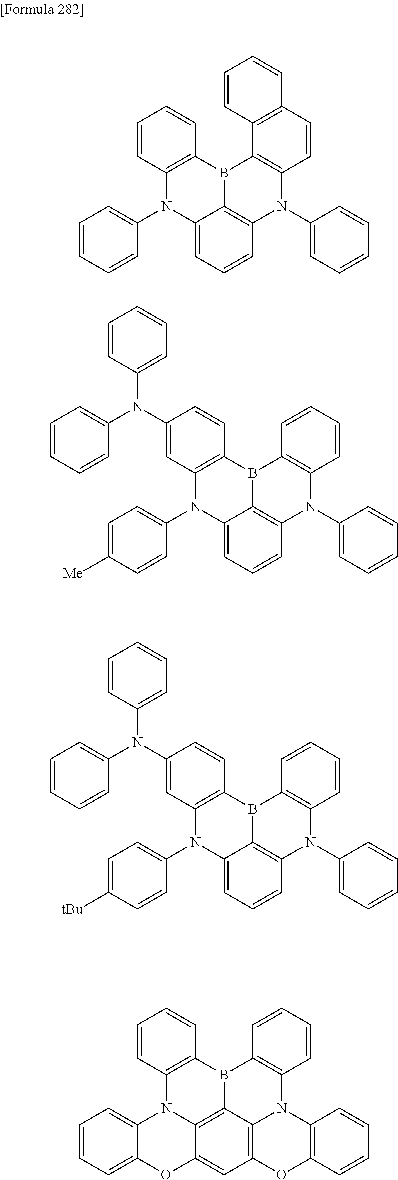

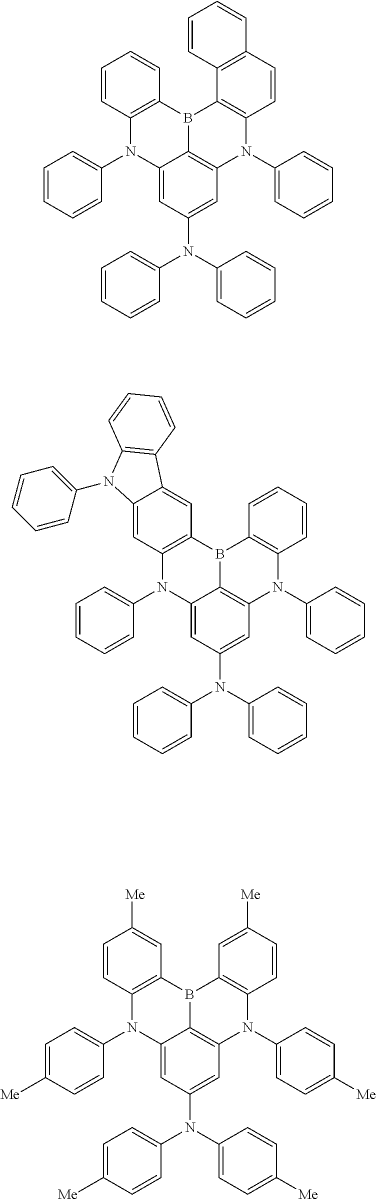 [SMILES: c1ccc(N(c2ccccc2)c2cc3c4c(c2)N(c2ccccc2)c2ccc5ccccc5c2B4c2ccccc2N3c2ccccc2)cc1 ; c1ccc(N(c2ccccc2)c2cc3c4c(c2)N(c2ccccc2)c2cc5c(cc2B4c2ccccc2N3c2ccccc2)c2ccccc2n5-c2ccccc2)cc1 ; Cc1ccc(N(c2ccc(C)cc2)c2cc3c4c(c2)N(c2ccc(C)cc2)c2ccc(C)cc2B4c2cc(C)ccc2N3c2ccc(C)cc2)cc1]

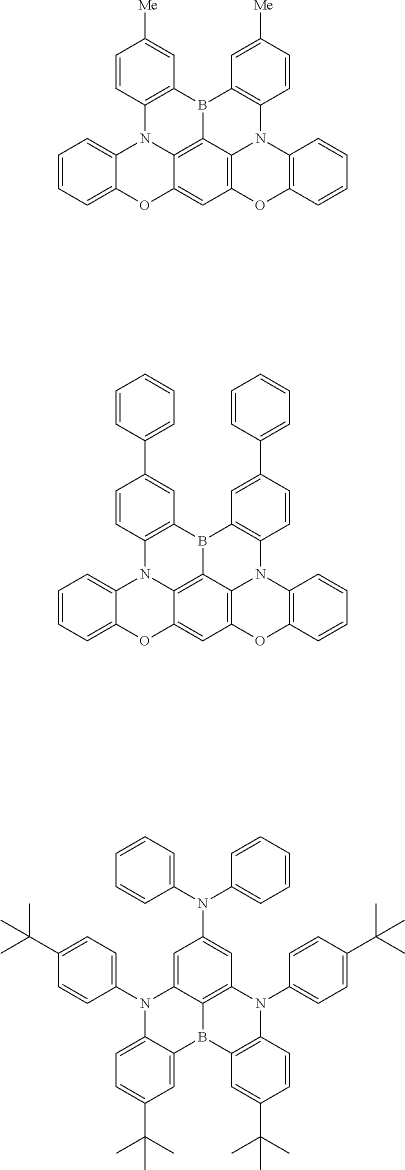 [SMILES: Cc1ccc2c(c1)B1c3cc(C)ccc3-n3c4ccccc4oc4cc5oc6ccccc6n-2c5c1c43 ; c1ccc(-c2ccc3c(c2)B2c4cc(-c5ccccc5)ccc4-n4c5ccccc5oc5cc6oc7ccccc7n-3c6c2c54)cc1 ; CC(C)(C)c1ccc(N2c3ccc(C(C)(C)C)cc3B3c4cc(C(C)(C)C)ccc4N(c4ccc(C(C)(C)C)cc4)c4cc(N(c5ccccc5)c5ccccc5)cc2c43)cc1]

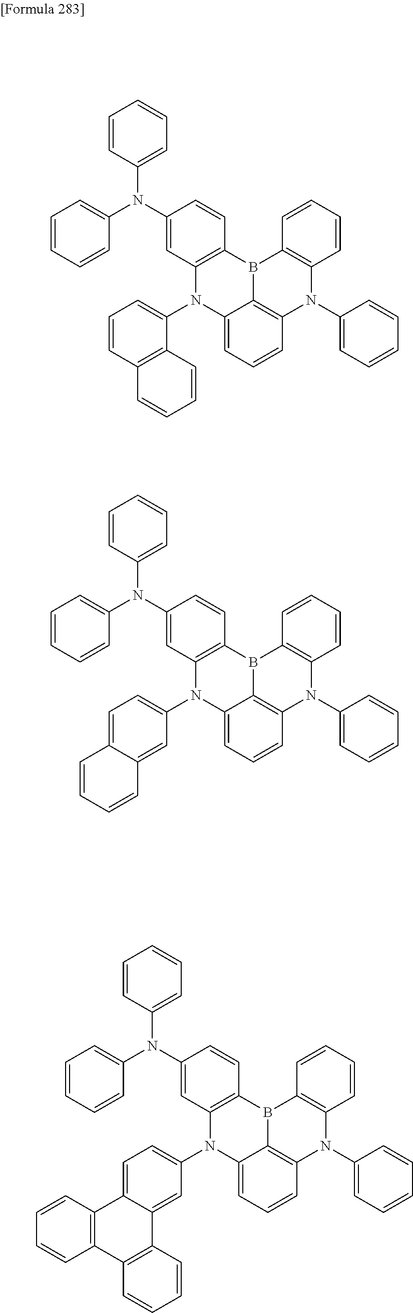 [SMILES: c1ccc(N(c2ccccc2)c2ccc3c(c2)N(c2cccc4ccccc24)c2cccc4c2B3c2ccccc2N4c2ccccc2)cc1 ; c1ccc(N(c2ccccc2)c2ccc3c(c2)N(c2ccc4ccccc4c2)c2cccc4c2B3c2ccccc2N4c2ccccc2)cc1 ; c1ccc(N(c2ccccc2)c2ccc3c(c2)N(c2ccc4c5ccccc5c5ccccc5c4c2)c2cccc4c2B3c2ccccc2N4c2ccccc2)cc1]

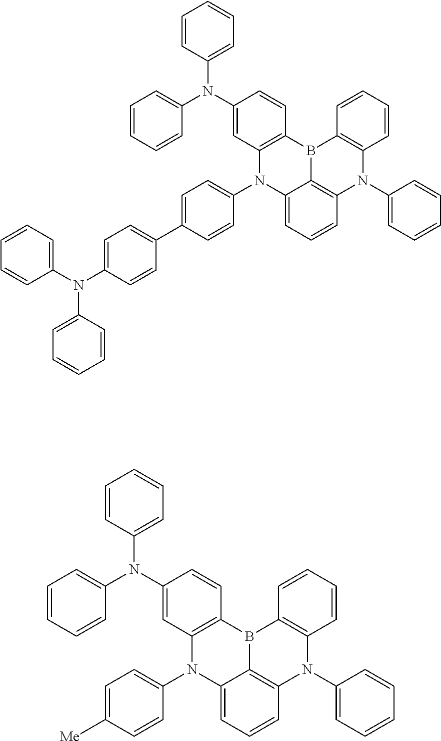 [SMILES: c1ccc(N(c2ccccc2)c2ccc(-c3ccc(N4c5cc(N(c6ccccc6)c6ccccc6)ccc5B5c6ccccc6N(c6ccccc6)c6cccc4c65)cc3)cc2)cc1 ; Cc1ccc(N2c3cc(N(c4ccccc4)c4ccccc4)ccc3B3c4ccccc4N(c4ccccc4)c4cccc2c43)cc1]

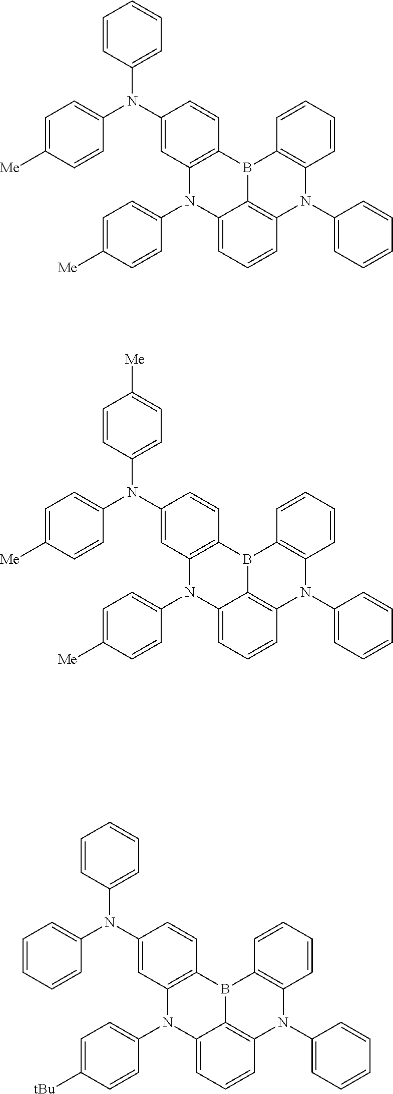 [SMILES: Cc1ccc(N(c2ccccc2)c2ccc3c(c2)N(c2ccc(C)cc2)c2cccc4c2B3c2ccccc2N4c2ccccc2)cc1 ; Cc1ccc(N(c2ccc(C)cc2)c2ccc3c(c2)N(c2ccc(C)cc2)c2cccc4c2B3c2ccccc2N4c2ccccc2)cc1 ; CC(C)(C)c1ccc(N2c3cc(N(c4ccccc4)c4ccccc4)ccc3B3c4ccccc4N(c4ccccc4)c4cccc2c43)cc1]

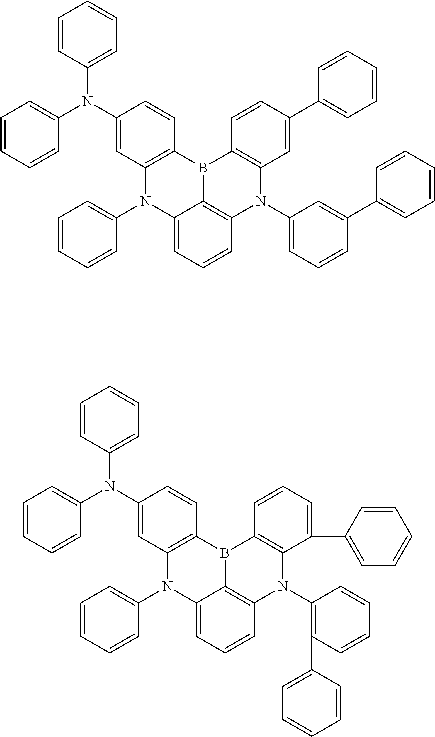 [SMILES: c1ccc(-c2cccc(N3c4cc(-c5ccccc5)ccc4B4c5ccc(N(c6ccccc6)c6ccccc6)cc5N(c5ccccc5)c5cccc3c54)c2)cc1 ; c1ccc(-c2ccccc2N2c3cccc4c3B(c3ccc(N(c5ccccc5)c5ccccc5)cc3N4c3ccccc3)c3cccc(-c4ccccc4)c32)cc1]

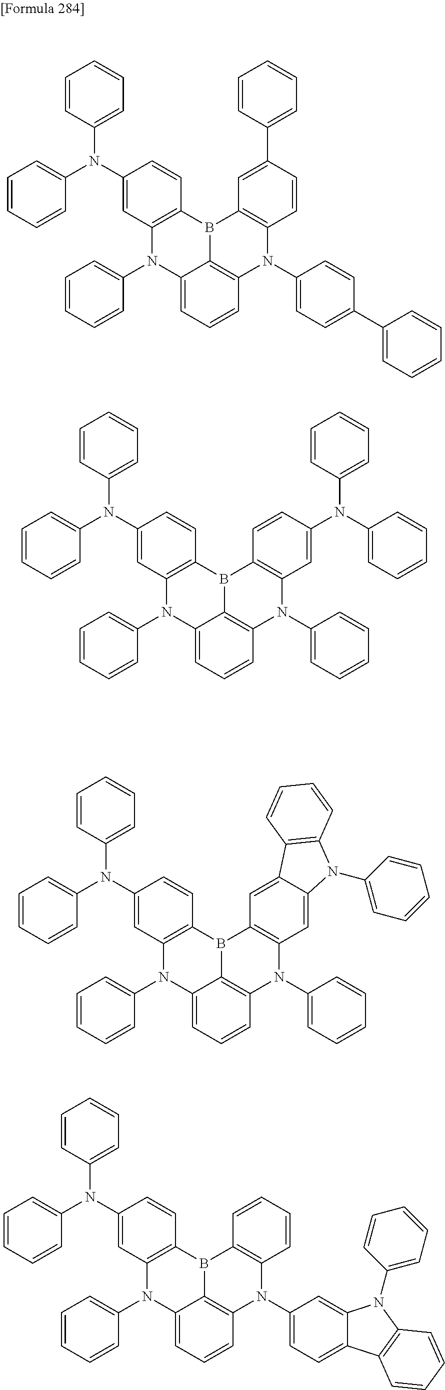 [SMILES: c1ccc(-c2ccc(N3c4ccc(-c5ccccc5)cc4B4c5ccc(N(c6ccccc6)c6ccccc6)cc5N(c5ccccc5)c5cccc3c54)cc2)cc1 ; c1ccc(N(c2ccccc2)c2ccc3c(c2)N(c2ccccc2)c2cccc4c2B3c2ccc(N(c3ccccc3)c3ccccc3)cc2N4c2ccccc2)cc1 ; c1ccc(N(c2ccccc2)c2ccc3c(c2)N(c2ccccc2)c2cccc4c2B3c2cc3c5ccccc5n(-c5ccccc5)c3cc2N4c2ccccc2)cc1 ; c1ccc(N(c2ccccc2)c2ccc3c(c2)N(c2ccccc2)c2cccc4c2B3c2ccccc2N4c2ccc3c4ccccc4n(-c4ccccc4)c3c2)cc1]

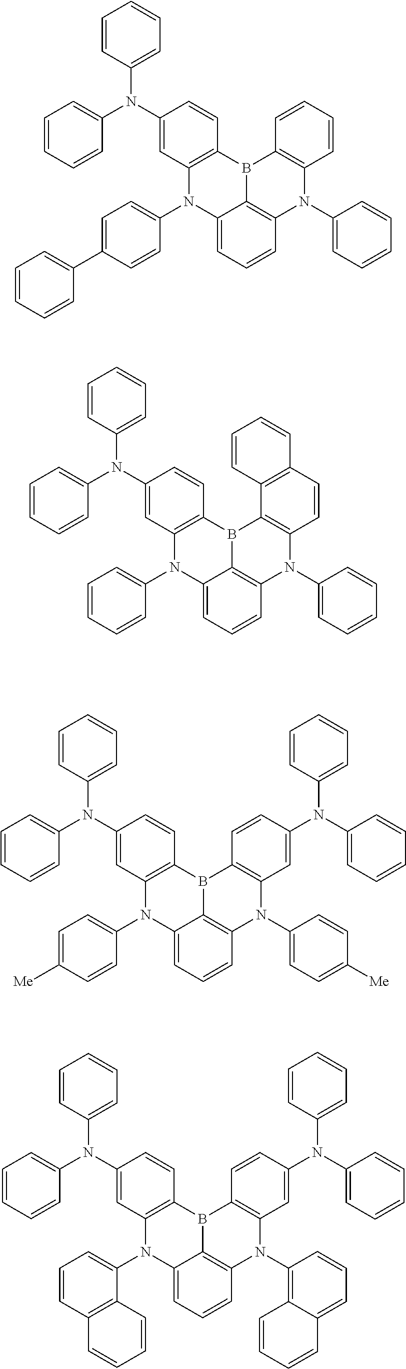 [SMILES: c1ccc(-c2ccc(N3c4cc(N(c5ccccc5)c5ccccc5)ccc4B4c5ccccc5N(c5ccccc5)c5cccc3c54)cc2)cc1 ; c1ccc(N(c2ccccc2)c2ccc3c(c2)N(c2ccccc2)c2cccc4c2B3c2c(ccc3ccccc23)N4c2ccccc2)cc1 ; Cc1ccc(N2c3cc(N(c4ccccc4)c4ccccc4)ccc3B3c4ccc(N(c5ccccc5)c5ccccc5)cc4N(c4ccc(C)cc4)c4cccc2c43)cc1 ; c1ccc(N(c2ccccc2)c2ccc3c(c2)N(c2cccc4ccccc24)c2cccc4c2B3c2ccc(N(c3ccccc3)c3ccccc3)cc2N4c2cccc3ccccc23)cc1]

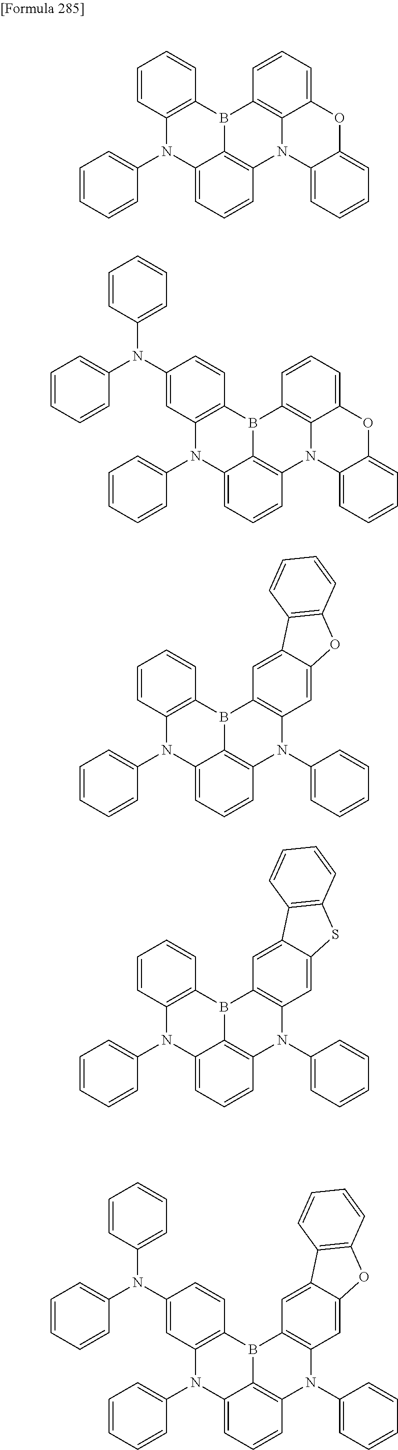

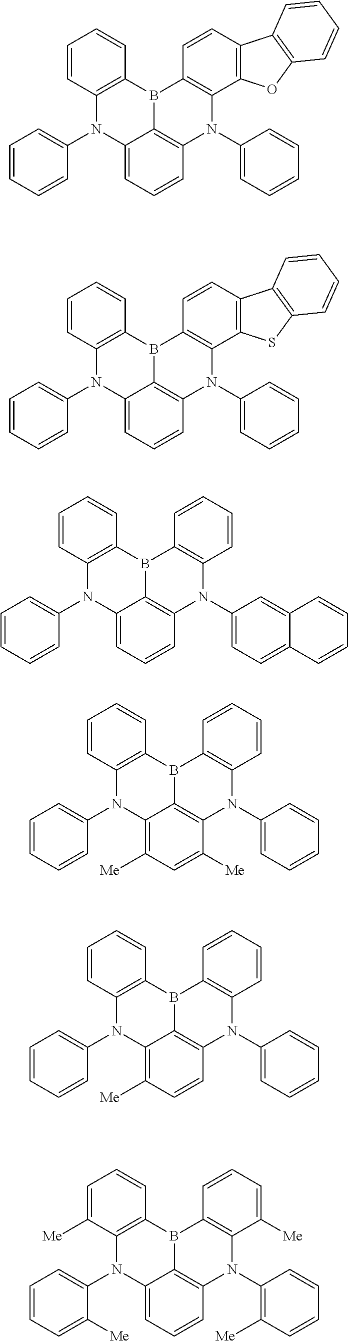 [SMILES: c1ccc(N2c3ccccc3B3c4ccc5c(oc6ccccc65)c4N(c4ccccc4)c4cccc2c43)cc1 ; c1ccc(N2c3ccccc3B3c4ccc5c(sc6ccccc65)c4N(c4ccccc4)c4cccc2c43)cc1 ; c1ccc(N2c3ccccc3B3c4ccccc4N(c4ccc5ccccc5c4)c4cccc2c43)cc1 ; Cc1cc(C)c2c3c1N(c1ccccc1)c1ccccc1B3c1ccccc1N2c1ccccc1 ; Cc1ccc2c3c1N(c1ccccc1)c1ccccc1B3c1ccccc1N2c1ccccc1 ; Cc1ccccc1N1c2cccc3c2B(c2cccc(C)c21)c1cccc(C)c1N3c1ccccc1C]

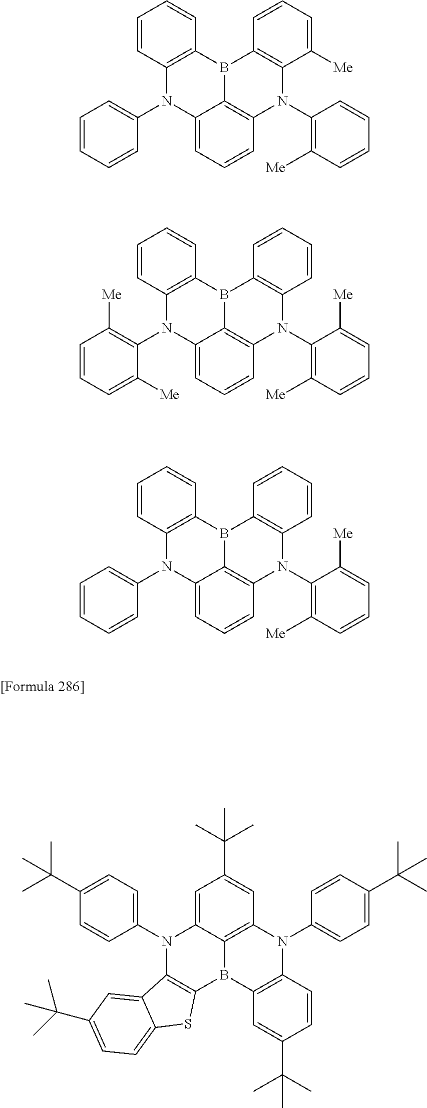 [SMILES: Cc1ccccc1N1c2cccc3c2B(c2ccccc2N3c2ccccc2)c2cccc(C)c21 ; Cc1cccc(C)c1N1c2ccccc2B2c3ccccc3N(c3c(C)cccc3C)c3cccc1c32 ; Cc1cccc(C)c1N1c2ccccc2B2c3ccccc3N(c3ccccc3)c3cccc1c32 ; CC(C)(C)c1ccc(N2c3ccc(C(C)(C)C)cc3B3c4sc5ccc(C(C)(C)C)cc5c4N(c4ccc(C(C)(C)C)cc4)c4cc(C(C)(C)C)cc2c43)cc1]

In an exemplary arrangement of the organic EL device of the exemplary embodiment, the emitting region consists of the first emitting layer.

In an exemplary arrangement of the organic EL device of the exemplary embodiment, the emitting region includes the first emitting layer and further includes the second emitting layer.

In an exemplary arrangement of the organic EL device of the exemplary embodiment, the emitting region consists of the first emitting layer and the second emitting layer.

In an exemplary arrangement of the organic EL device of the exemplary embodiment, the emitting region may include an organic layer different from the first emitting layer and the second emitting layer.

In an exemplary arrangement of the organic EL device according to the exemplary embodiment, the emitting region includes at least the first emitting layer containing the first host material and the first additional host material and the second emitting layer containing the second host material. The first host material and the second host material are different from each other, and the first additional host material and the second host material are different from each other.

When the emitting region includes at least the first emitting layer and the second emitting layer, the luminous efficiency is improvable by utilizing triplet-triplet-annihilation (occasionally referred to as TTA).

TTA is a mechanism in which triplet excitons collide with one another to generate singlet excitons. It should be noted that the TTA mechanism is also occasionally referred to as a TTF mechanism as described in WO2010/134350.

The TTF phenomenon will be described. Holes injected from an anode and electrons injected from a cathode are recombined in an emitting layer to generate excitons. As for the spin state, as is conventionally known, singlet excitons account for 25% and triplet excitons account for 75%. In a conventionally known fluorescent device, light is emitted when singlet excitons of 25% are relaxed to the ground state. The remaining triplet excitons of 75% are returned to the ground state without emitting light through a thermal deactivation process. Accordingly, the theoretical limit value of the internal quantum efficiency of the conventional fluorescent device is believed to be 25%.

The behavior of triplet excitons generated within an organic substance has been theoretically examined. According to S. M. Bachilo et al. (J. Phys. Chem. A, 104, 7711 (2000)), assuming that high-order excitons such as quintet excitons are quickly returned to triplet excitons, triplet excitons (hereinafter abbreviated as ³A*) collide with one another with an increase in density thereof, whereby a reaction shown by the following formula occurs. In the formula, ¹A represents the ground state and ¹A* represents the lowest singlet excitons.

$${}^{3} {A}^{*} + {}^{3} {A}^{*} \to {( 4 / 9 )}^{1} A + {( 1 / 9 )}^{1} {A}^{*} + {( 13 / 9 )}^{3} {A}^{*}$$

In other words, 5³A*→4¹A+1A* is satisfied, and it is expected that, among triplet excitons initially generated, which account for 75%, one fifth thereof (i.e., 20%) is changed to singlet excitons. Accordingly, the amount of singlet excitons which contribute to emission is 40%, which is a value obtained by adding 15% (75%×(⅕)=15%) to 25%, which is the amount ratio of initially generated singlet excitons. At this time, a ratio of luminous intensity derived from TTF (TTF ratio) relative to the total luminous intensity is 15/40, i.e., 37.5%. Assuming that singlet excitons are generated by collision of initially generated triplet excitons accounting for 75% (i.e., one singlet exciton is generated from two triplet excitons), a significantly high internal quantum efficiency of 62.5% is obtained, which is a value obtained by adding 37.5% (75%×(½)=37.5%) to 25% (the amount ratio of initially generated singlet excitons). At this time, the TTF ratio is 37.5/62.5=60%.

In an exemplary arrangement of the organic EL device according to the exemplary embodiment, in terms of expressing the TTF mechanism, a triplet energy of the first host material or the first additional host material T₁ and a triplet energy of the second host material T₁(H2) preferably satisfy a relationship of a numerical formula (Numerical Formula 1) below, more preferably satisfy a relationship of a numerical formula (Numerical Formula 2) below.

In the following, the triplet energy of the first host material or the first additional host malarial T₁ is collectively referred to as T₁(H1).

$${T}_{1} ( H2 ) > {T}_{1} ( H 1 ) \qquad ( Numerical Formula 1 )$$

$${T}_{1} ( H2 ) - {T}_{1} ( H 1 ) > 0.03 eV \qquad ( Numerical Formula 2 )$$

When an exemplary arrangement of the organic EL device according to the exemplary embodiment includes the first emitting layer and the second emitting layer satisfying the relationship of the above numerical formula (Numerical Formula 1), luminous efficiency of the device is improvable.

In an exemplary arrangement of the organic EL device according to the exemplary embodiment, it is considered that when the relationship of the numerical formula (Numerical Formula 1) is satisfied, triplet excitons generated by recombination of holes and electrons in the second emitting layer and present on an interface between the second emitting layer and organic layer(s) in direct contact therewith are not likely to be quenched even under the presence of excessive carriers on the interface between the second emitting layer and the organic layer(s). For instance, the presence of a recombination region locally on an interface between the second emitting layer and a hole transporting layer or an electron blocking layer is considered to cause quenching by excessive electrons. Meanwhile, the presence of a recombination region locally on an interface between the second emitting layer and an electron transporting layer or a hole blocking layer is considered to cause quenching by excessive holes.

In an exemplary arrangement of the organic EL device according to the exemplary embodiment, by including the first emitting layer and the second emitting layer so as to satisfy the relationship of the numerical formula (Numerical Formula 1), triplet excitons generated in the second emitting layer can transfer to the first emitting layer without being quenched by excessive carriers and be inhibited from back-transferring from the first emitting layer to the second emitting layer. Consequently, the first emitting layer exhibits the TTF mechanism to effectively generate singlet excitons, thereby improving the luminous efficiency.

Accordingly, the organic EL device includes, as different regions, the second emitting layer mainly generating triplet excitons and the first emitting layer mainly exhibiting the TTF mechanism using triplet excitons having transferred from the second emitting layer, and has a difference in triplet energy provided by using a compound having a smaller triplet energy than that of the second host material in the second emitting layer as the first host material and the first additional host material in the first emitting layer. The luminous efficiency is thus improved.

In an exemplary arrangement of the organic EL device according to the exemplary embodiment, the first emitting layer and the second emitting layer are in direct contact with each other.

Herein, a layer arrangement in which “the first emitting layer and the second emitting layer are in direct contact with each other” may include one of embodiments (LS1), (LS2), and (LS3) below.

(LS1) An embodiment in which a region containing both the first host material, the first additional host material, and the second host material is generated in a process of vapor-depositing the compound of the first emitting layer and vapor-depositing the compound of the second emitting layer, and is present on the interface between the first emitting layer and the second emitting layer.

(LS2) An embodiment in which in a case of containing a luminescent compound in the first emitting layer and the second emitting layer, a region containing the first host material, the first additional host material, the second host material, and the luminescent compound is generated in a process of vapor-depositing the compound of the first emitting layer and vapor-depositing the compound of the second emitting layer, and is present on the interface between the first emitting layer and the second emitting layer.

(LS3) An embodiment in which in a case of containing a luminescent compound in the first emitting layer and the second emitting layer, a region containing the luminescent compound, a region containing the first host material and the first additional host material or a region containing the second host material is generated in a process of vapor-depositing the compound of the first emitting layer and vapor-depositing the compound of the second emitting layer, and is present on the interface between the first emitting layer and the second emitting layer.

### Second Emitting Layer

In an exemplary arrangement of the organic EL device according to the exemplary embodiment, the second emitting layer contains the second host material. Although the second host material is not particularly limited, for instance, a compound selected from the group consisting of the first compound described below and a compound represented by the formula (H10) is usable.

In an exemplary arrangement of the organic EL device according to the exemplary embodiment, the second emitting layer contains a second luminescent compound. Although the second luminescent compound is not particularly limited, for instance, a compound selected from the group consisting of a compound represented by the formula (6), the third compound described below, and the fourth compound described below is usable.

In an exemplary arrangement of the organic EL device according to the exemplary embodiment, the second emitting layer contains the second host material and the second luminescent compound.

In an exemplary arrangement of the organic EL device of the exemplary embodiment, the second luminescent compound is a compound that is the same as or different from the first luminescent compound contained in the first emitting layer.

In an exemplary arrangement of the organic EL device of the exemplary embodiment, the second host material and the first host material contained in the first emitting layer are mutually different compounds.

In an exemplary arrangement of the organic EL device of the exemplary embodiment, the second host material and the first additional host material contained in the first emitting layer are mutually different compounds.

The second luminescent compound is preferably a compound that emits light having a maximum peak wavelength of 500 nm or less, more preferably a compound that emits light having a maximum peak wavelength in a range from 430 nm to 480 nm. The second luminescent compound is preferably a fluorescent compound that emits fluorescence having a maximum peak wavelength of 500 nm or less, more preferably a fluorescent compound that emits fluorescence having a maximum peak wavelength in a range from 430 nm to 480 nm.

In an exemplary arrangement of the organic EL device according to the exemplary embodiment, the second emitting layer contains the second host material and the second luminescent compound that emits light having a maximum peak wavelength of 500 nm or less.

In an exemplary arrangement of the organic EL device according to the exemplary embodiment, the full width at half maximum of the maximum peak of the second luminescent compound is in a range from 1 nm to 30 nm.

A method of measuring the maximum peak wavelength of the compound is as follows.

In an exemplary arrangement of the organic EL device according to the exemplary embodiment, a triplet energy of the first luminescent compound T₁(D1) and the triplet energy of the first host material or the first additional host material T₁(H1) preferably satisfy a relationship of a numerical formula (Numerical Formula 4A) below.

$${T}_{1} ( D 1 ) > {T}_{1} ( H 1 ) \qquad ( Numerical Formula 4 A )$$

In an exemplary arrangement of the organic EL device according to the exemplary embodiment, when the first luminescent compound and the first host material or the first additional host material satisfy the relationship of the numerical formula (Numerical Formula 4A), in transfer of triplet excitons generated in the second emitting layer to the first emitting layer, the triplet excitons energy-transfer not onto the first luminescent compound having higher triplet energy but onto molecules of the first host material or the first additional host material. In addition, triplet excitons generated by recombination of holes and electrons on the first host material or the first additional host material do not transfer to the first luminescent compound having higher triplet energy. Triplet excitons generated by recombination on molecules of the first luminescent compound quickly energy-transfer to molecules of the first host material or the first additional host material.

Triplet excitons in the first host material or the first additional host material do not transfer to the first luminescent compound but efficiently collide with one another on the first host material or the first additional host material to generate singlet excitons by the TTF phenomenon.

In an exemplary arrangement of the organic EL device according to the exemplary embodiment, a singlet energy of the first host material or the first additional host material S₁ and a singlet energy of the first luminescent compound S₁(D1) preferably satisfy a relationship of a numerical formula (Numerical Formula 4) below. In the following, the singlet energy of the first host material or the first additional host malarial S₁ is collectively referred to as S₁(H1).

$${S}_{1} ( {H}_{1} ) > {S}_{1} ( D 1 ) \qquad ( Numerical Formula 4 )$$

In an exemplary arrangement of the organic EL device according to the exemplary embodiment, when the first luminescent compound and the first host material or the first additional host material satisfy the relationship of the numerical formula (Numerical Formula 4), due to the singlet energy of the first luminescent compound being smaller than the singlet energy of the first host material or the first additional host material, singlet excitons generated by the TTF phenomenon energy-transfer from the first host material or the first additional host material to the first luminescent compound, thereby contributing to emission (preferably fluorescence) of the first luminescent compound.

In an exemplary arrangement of the organic EL device according to the exemplary embodiment, when the second emitting layer and the first emitting layer are layered in this order from a side close to the anode, it is preferable that an electron mobility of the second host material μe(H2) and an electron mobility of the first host material or the first additional host material pe satisfy a relationship of the formula (Numerical Formula 3) below. When the first host material or the first additional host material and the second host material satisfy the relationship of the numerical formula (Numerical Formula 3) below, a recombination ability between holes and electrons in the second emitting layer improves.

In the following, the electron mobility of the first host material or the first additional host malarial pe is collectively referred to as μe(H1).

$$μ e ( H 1 ) > μ e ( H 2 ) \qquad ( Numerical Formula 3 )$$

In an exemplary arrangement of the organic EL device according to the exemplary embodiment, when the second emitting layer and the first emitting layer are layered in this order from a side close to the anode, it is also preferable that a hole mobility of the second host material μh(H2) and a hole mobility of the first host material or the first additional host material ph satisfy a relationship of a formula (Numerical Formula 31) below.

In the following, the hole mobility of the first host material or the first additional host malarial μh is collectively referred to as μh(H1).

$$μ h ( H 2 ) > μ h ( H 1 ) \qquad ( Numerical Formula 31 )$$

In an exemplary arrangement of the organic EL device according to the exemplary embodiment, when the second emitting layer and the first emitting layer are layered in this order from a side close to the anode, it is also preferable that the hole mobility of the second host material μh(H2), the electron mobility of the second host material μe(H2), the hole mobility of the first host material or the first additional host material μh(H1), and the electron mobility of the first host material or the first additional host material μe(H1) satisfy a relationship of a numerical formula (Numerical Formula 32) below.

$$( μ e ( H 1 ) / μ h ( H 1 ) ) > ( μ e ( H 2 ) / μ h ( H 2 ) ) \qquad ( Numerical Formula 32 )$$

In an exemplary arrangement of the organic EL device according to the exemplary embodiment, it is preferable that a singlet energy of the second host material S₁(H2) and a singlet energy of the second luminescent compound S₁(D2) satisfy a relationship of a numerical formula (Numerical Formula 20) below.

$${S}_{1} ( H 2 ) > {S}_{1} ( D 2 ) \qquad ( Numerical Formula 20 )$$

When the second host material and the second luminescent compound satisfy the relationship of the numerical formula (Numerical Formula 20), singlet excitons generated on the second host material easily energy-transfer from the second host material to a second dopant material, thereby contributing to emission (preferably, fluorescence) of the second luminescent compound.

In the organic EL device according to a second exemplary embodiment, the triplet energy of the second host material T₁(H2) and a triplet energy of the second luminescent compound T₁(D2) preferably satisfy a relationship of a numerical formula (Numerical Formula 20A) below.

$${T}_{1} ( D 2 ) > {T}_{1} ( H 2 ) \qquad ( Numerical Formula 20 A )$$

When the second host material and the second luminescent compound satisfy the relationship of the numerical formula (Numerical Formula 20A), triplet excitons generated in the second emitting layer are transferred not onto the second luminescent compound having higher triplet energy but onto the second host material, thereby being easily transferred to the first emitting layer.

Methods of measuring the triplet energy T₁, the singlet energy S₁, the hole mobility and the electron mobility will be described below.

In an exemplary arrangement of the organic EL device according to the exemplary embodiment, the second luminescent compound is a compound containing no azine ring structure in a molecule.

In an exemplary arrangement of the organic EL device according to the exemplary embodiment, the second luminescent compound is preferably not a boron-containing complex, more preferably not a complex.

As a compound that emits blue fluorescence and is usable for the second emitting layer, for instance, compounds such as a pyrene derivative, a styrylamine derivative, a chrysene derivative, a fluoranthene derivative, a fluorene derivative, a diamine derivative, and a triarylamine derivative are usable.

In an exemplary arrangement of the organic EL device according to the exemplary embodiment, the second emitting layer contains no metal complex. In an exemplary arrangement of the organic EL device according to the exemplary embodiment, the second emitting layer contains no boron-containing complex.

In an exemplary arrangement of the organic EL device according to the exemplary embodiment, the second emitting layer contains no phosphorescent material (dopant material).

In an exemplary arrangement of the organic EL device according to the exemplary embodiment, the second emitting layer contains neither a heavy-metal complex nor a phosphorescent rare earth metal complex.

In an exemplary arrangement of the organic EL device according to the exemplary embodiment, the second emitting layer contains 0.5 mass % or more of the luminescent compound with respect to a total mass of the second emitting layer. The second emitting layer contains the luminescent compound preferably at 10 mass % or less, more preferably at 7 mass % or less, and still more preferably at 5 mass % less with respect to the total mass of the second emitting layer.

In an exemplary arrangement of the organic EL device according to the exemplary embodiment, the second emitting layer preferably contains the second host material at 60 mass % or more, more preferably at 70 mass % or more, still more preferably at 80 mass % or more, still further more preferably at 90 mass % or more, and yet still further more preferably at 95 mass % or more, with respect to the total mass of the second emitting layer.

The second emitting layer preferably contains the second host material at 99.5 mass % or less with respect to the total mass of the second emitting layer.

When the second emitting layer contains the second host material and the second luminescent compound, the upper limit of the total of the content ratios of the second host material and the second luminescent compound is 100 mass %.

In the organic EL device according to the exemplary embodiment, the second emitting layer may further contain any other material than the second host material and the second luminescent compound.

The second emitting layer may contain a single type of the second host material or may contain two or more types of the second host material. The second emitting layer may contain a single type of the second luminescent compound or may contain two or more types of the second luminescent compound.

In an exemplary arrangement of the organic EL device according to the exemplary embodiment, a film thickness of the first emitting layer is preferably 5 nm or more, more preferably 15 nm or more. When the film thickness of the first emitting layer is 5 nm or more, in an arrangement in which the emitting region includes the second emitting layer, triplet excitons having transferred from the second emitting layer to the first emitting layer are easily inhibited from returning to the second emitting layer. Further, when the film thickness of the first emitting layer is 5 nm or more, triplet excitons can be sufficiently separated from the recombination portion in the second emitting layer.

In an exemplary arrangement of the organic EL device according to the exemplary embodiment, the film thickness of the first emitting layer is preferably 20 nm or less. When the film thickness of the first emitting layer is 20 nm or less, a density of the triplet excitons in the first emitting layer is improved to cause the TTF phenomenon more easily.

In an exemplary arrangement of the organic EL device according to the exemplary embodiment, the film thickness of the first emitting layer is preferably in a range from 5 nm to 20 nm.

In an exemplary arrangement of the organic EL device according to the exemplary embodiment, the film thickness of the second emitting layer is preferably 3 nm or more, more preferably 5 nm or more. When the film thickness of the second emitting layer is 3 nm or more, the film thickness is sufficiently large to cause recombination of holes and electrons in the second emitting layer.

In an exemplary arrangement of the organic EL device according to the exemplary embodiment, the film thickness of the second emitting layer is preferably 15 nm or less, more preferably 10 nm or less. When the film thickness of the second emitting layer is 15 nm or less, the film thickness is sufficiently thin to allow for transfer of triplet excitons to the first emitting layer.

In an exemplary arrangement of the organic EL device according to the exemplary embodiment, the film thickness of the second emitting layer is more preferably in a range from 3 nm to 15 nm.

### First Host Material and Second Host Material

In an exemplary arrangement of the organic EL device according to the exemplary embodiment, the first host material is also preferably a compound except for the exemplary compounds above (such as the compounds represented by the formulae (H10) and (H20)). For instance, the first host material is also preferably a compound selected from the group consisting of the first compound represented by a formula (1), a formula (1X), a formula (12X), a formula (13X), a formula (14X), a formula (15X), a formula (16X), a formula (1000B), a formula (16X), a formula (17X-1), a formula (17X-2), a formula (17X-3) or a formula (18) below.

In an exemplary arrangement of the organic EL device according to the exemplary embodiment, the second host material is preferably, for instance, a compound selected from the group consisting of the first compound represented by the formula (1), the formula (1X), the formula (12X), the formula (13X), the formula (14X), the formula (15X), the formula (16X), the formula (1000B), the formula (16X), the formula (17X-1), the formula (17X-2), the formula (17X-3), or the formula (18) below, a compound represented by the formula (H10), and a compound represented by the formula (H20).

The first compound may also be used as the first host material, the first additional host material, and the second host material.

### Compound Represented by Formula (1)

In an exemplary arrangement of the organic EL device according to the exemplary embodiment, the first compound is a compound represented by the formula (1) below. The first compound represented by the formula (1) has at least one group represented by a formula (11) below.

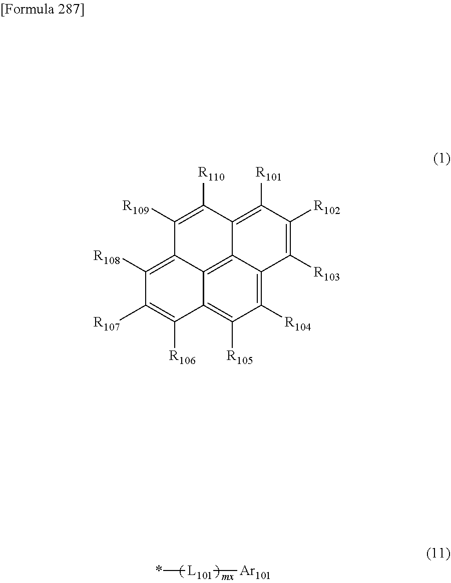

In the formula (1):
R₁₀₁ to R₁₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (11);
at least one of R₁₀₁ to R₁₁₀ is a group represented by the formula (11);
when a plurality of groups represented by the formula (11) are present, the plurality of groups represented by the formula (11) are mutually the same or different;
L₁₀₁ is a single bond, a substituted or unsubstituted arylene group having 6 to 50 ring carbon atoms, or a substituted or unsubstituted divalent heterocyclic group having 5 to 50 ring atoms;
Ar₁₀₁ is a substituted or unsubstituted aryl group having 6 to 50 ring carbon atoms, or a substituted or unsubstituted heterocyclic group having 5 to 50 ring atoms;
mx is 0, 1, 2, 3, 4, or 5;
when two or more L₁₀₁ are present, the two or more L₁₀₁ are mutually the same or different;
when two or more Ar₁₀₁ are present, the two or more Ar₁₀₁ are mutually the same or different; and
* in the formula (11) represents a bonding position to a pyrene ring represented by the formula (1).

In the first compound represented by the formula (1), R₉₀₁, R₉₀₂, R₉₀₃, R₉₀₄, R₉₀₅, R₉₀₆, R₉₀₇, R₈₀₁ and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different;
when a plurality of R₉₀₇ are present, the plurality of R₉₀₇ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

In an exemplary embodiment, Ar₁₀₁ is preferably a substituted or unsubstituted aryl group having 6 to 50 carbon atoms.

In an exemplary embodiment, Ar₁₀₁ is preferably a substituted or unsubstituted phenyl group, a substituted or unsubstituted naphthyl group, a substituted or unsubstituted biphenyl group, a substituted or unsubstituted terphenyl group, a substituted or unsubstituted pyrenyl group, a substituted or unsubstituted phenanthryl group, or a substituted or unsubstituted fluorenyl group.

In an exemplary embodiment, the first compound is preferably represented by a formula (101) below.

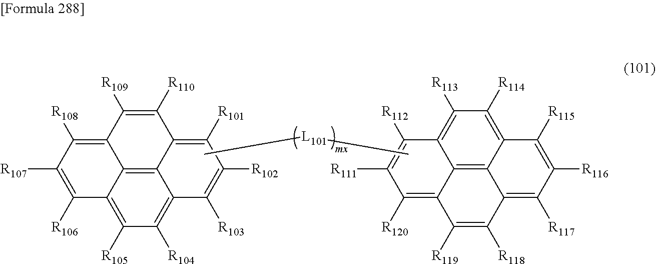

In the formula (101):
R₁₀₁ to R₁₂₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
one of R₁₀₁ to R₁₁₀ represents a bonding position to L₁₀₁, and one of R₁₁₁ to R₁₂₀ represents a bonding position to L₁₀₁;
L₁₀₁ is a single bond, a substituted or unsubstituted arylene group having 6 to 50 ring carbon atoms, or a substituted or unsubstituted divalent heterocyclic group having 5 to 50 ring atoms;
mx is 0, 1, 2, 3, 4, or 5; and
when two or more L₁₀₁ are present, the two or more L₁₀₁ are mutually the same or different.

In an exemplary embodiment, L₁₀₁ is preferably a single bond or a substituted or unsubstituted arylene group having 6 to 50 ring carbon atoms.

In an exemplary embodiment, two or more of R₁₀₁ to R₁₁₀ are each preferably a group represented by the formula (11).

In an exemplary embodiment, it is preferable that two or more of R₁₀₁ to R₁₁₀ are each a group represented by the formula (11) and Ar₁₀₁ is a substituted or unsubstituted aryl group having 6 to 50 ring carbon atoms.

In an exemplary embodiment: it is preferable that: Ar₁₀₁ is not a substituted or unsubstituted pyrenyl group, L₁₀₁ is not a substituted or unsubstituted pyrenylene group, and the substituted or unsubstituted aryl group having 6 to 50 ring carbon atoms for R₁₀₁ to R₁₁₀ not being the group represented by the formula (11) is not a substituted or unsubstituted pyrenyl group.

In an exemplary embodiment: R₁₀₁ to R₁₁₀ not being the group represented by the formula (11) are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

In an exemplary embodiment: R₁₀₁ to R₁₁₀ not being the group represented by the formula (11) are preferably each independently a hydrogen atom, a substituted or unsubstituted alkyl group having 1 to 50 carbon atoms, or a substituted or unsubstituted cycloalkyl group having 3 to 50 ring carbon atoms.

In an exemplary embodiment, R₁₀₁ to R₁₁₀ not being the group represented by the formula (11) are each preferably a hydrogen atom.

### Compound Represented by Formula (1X)

In an exemplary arrangement of the organic EL device according to the exemplary embodiment, the first compound is a compound represented by the formula (1X) below.

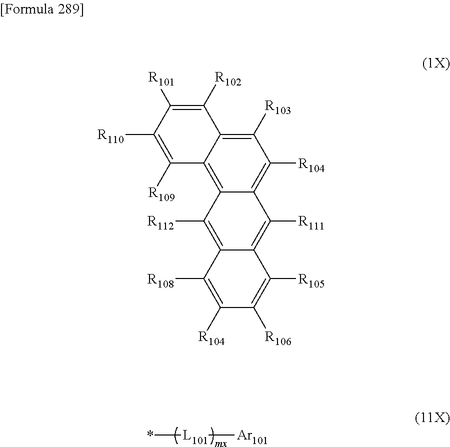

In the formula (1X):
R₁₀₁ to R₁₁₂ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (11X);
at least one of R₁₁ to R₁₁₂ is a group represented by the formula (11X);
when a plurality of groups represented by the formula (11X) are present, the plurality of groups represented by the formula (11X) are mutually the same or different;
L₁₀₁ is a single bond, a substituted or unsubstituted arylene group having 6 to 50 ring carbon atoms, or a substituted or unsubstituted divalent heterocyclic group having 5 to 50 ring atoms;
Ar₁₀₁ is a substituted or unsubstituted aryl group having 6 to 50 ring carbon atoms, or a substituted or unsubstituted heterocyclic group having 5 to 50 ring atoms;
mx is 1, 2, 3, 4, or 5;
when two or more L₁₀₁ are present, the two or more L₁₀₁ are mutually the same or different;
when two or more Ar₁₀₁ are present, the two or more Ar₁₀₁ are mutually the same or different; and
* in the formula (11X) represents a bonding position to a benz[a]anthracene ring in the formula (1X).

In an exemplary arrangement of the organic EL device of the exemplary embodiment, the group represented by the formula (11X) is a group represented by a formula (111X) below.

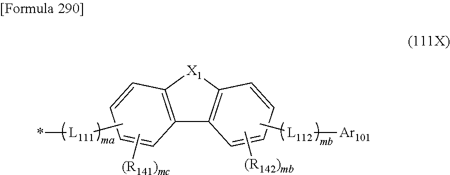

In the formula (111X):
X₁ is CR₁₄₃R₁₄₄, an oxygen atom, a sulfur atom, or NR₁₄₅;
L₁₁₁ and L₁₁₂ are each independently a single bond, a substituted or unsubstituted arylene group having 6 to 50 ring carbon atoms, or a substituted or unsubstituted divalent heterocyclic group having 5 to 50 ring atoms;
ma is 1, 2, 3 or 4;
mb is 1, 2, 3 or 4;
ma+mb is 2, 3, or 4;
Ar₁₀₁ represents the same as Ar₁₀₁ in the formula (11X);
R₁₄₁, R₁₄₂, R₁₄₃, R₁₄₄ and R₁₄₅ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
mc is 3;
three R₁₄₁ are mutually the same or different;
md is 3; and
three R₁₄₂ are mutually the same or different.

Among positions *1 to *8 of carbon atoms in a cyclic structure represented by a formula (111aX) below in the group represented by the formula (111X), L₁₁₁ is bonded to one of the positions *1 to *4, R₁₄₁ is bonded to each of three positions of the rest of *1 to *4, L₁₁₂ is bonded to one of the positions *5 to *8, and R₁₄₂ is bonded to each of three positions of the rest of *5 to *8.

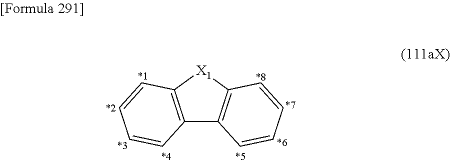

For instance, the group represented by the formula (111X), in which L₁₁₁ is bonded to a carbon atom at *2 in the cyclic structure represented by the formula (111aX) and L₁₁₂ is bonded to a carbon atom at *7 in the cyclic structure represented by the formula (111aX), is represented by a formula (111 bX) below.

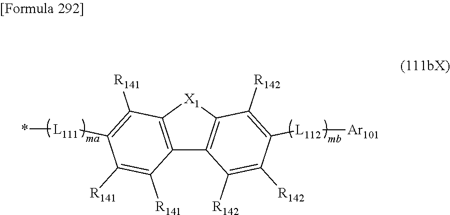

In the formula (111bX):
X₁, L₁₁₁, L₁₁₂, ma, mb, Ar₁₀₁, R₁₄₁, R₁₄₂, R₁₄₃, R₁₄₄ and R₁₄₅ each independently represent the same as X₁, L₁₁₁, L₁₁₂, ma, mb, Ar₁₀₁, R₁₄₁, R₁₄₂, R₁₄₃, R₁₄₄ and R₁₄₅ in the formula (111X);
a plurality of R₁₄₁ are mutually the same or different; and
a plurality of R₁₄₂ are mutually the same or different.

In the organic EL device according to the exemplary embodiment, the group represented by the formula (111X) is preferably a group represented by the formula (111bX).

In the compound represented by the formula (1X), preferably, ma is 1 or 2 and mb is 1 or 2.

In the compound represented by the formula (1X), preferably, ma is 1 and mb is 1.

In the compound represented by the formula (1X), Ar₁₀₁ is preferably a substituted or unsubstituted aryl group having 6 to 50 ring carbon atoms.

In the compound represented by the formula (1X), Ar₁₀₁ is preferably a substituted or unsubstituted phenyl group, a substituted or unsubstituted naphthyl group, a substituted or unsubstituted biphenyl group, a substituted or unsubstituted terphenyl group, a substituted or unsubstituted benz[a]anthryl group, a substituted or unsubstituted pyrenyl group, a substituted or unsubstituted phenanthryl group, or a substituted or unsubstituted fluorenyl group.

The compound represented by the formula (1X) is also preferably represented by a formula (101X) below.

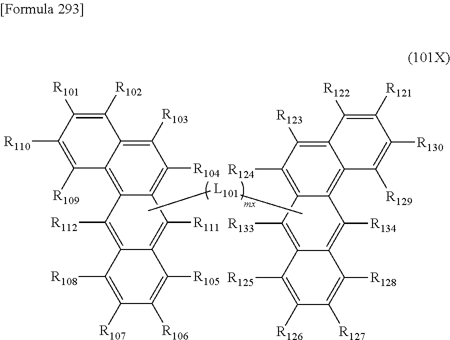

In the formula (101X):
one of R₁₁₁ and R₁₁₂ represents a bonding position to L₁₀₁ and one of R₁₃₃ and R₁₃₄ represents a bonding position to L₁₀₁;
R₁₀₁ to R₁₁₀, R₁₂₁ to R₁₃₀, R₁₁₁ or R₁₁₂ not being the bonding position to L₁₀₁, and R₁₃₃ or R₁₃₄ not being the bonding position to L₁₀₁ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
L₁₀₁ is a single bond, a substituted or unsubstituted arylene group having 6 to 50 ring carbon atoms, or a substituted or unsubstituted divalent heterocyclic group having 5 to 50 ring atoms;
mx is 1, 2, 3, 4, or 5; and
when two or more L₁₀₁ are present, the two or more L₁₀₁ are mutually the same or different.

In the compound represented by the formula (1X), L₁₀₁ is preferably a single bond, or a substituted or unsubstituted arylene group having 6 to 50 ring carbon atoms.

The compound represented by the formula (1X) is also preferably represented by a formula (102X) below.

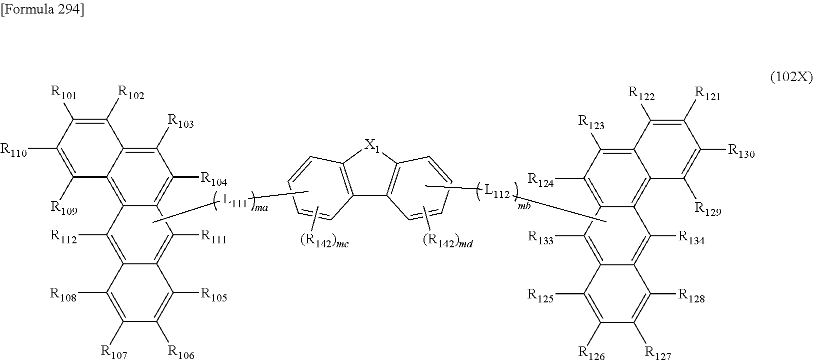

In the formula (102X):
one of R₁₁₁ and R₁₁₂ represents a bonding position to L₁₁₁ and one of R₁₃₃ and R₁₃₄ represents a bonding position to L₁₁₂;
R₁₀₁ to R₁₁₀, R₁₂₁ to R₁₃₀, R₁₁₁ or R₁₁₂ not being the bonding position to L₁₁₁, and R₁₃₃ or R₁₃₄ not being the bonding position to L₁₁₂ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
X₁ is CR₁₄₃R₁₄₄, an oxygen atom, a sulfur atom, or NR₁₄₅;
L₁₁₁ and L₁₁₂ are each independently a single bond, a substituted or unsubstituted arylene group having 6 to 50 ring carbon atoms, or a substituted or unsubstituted divalent heterocyclic group having 5 to 50 ring atoms;
ma is 1, 2, 3 or 4;
mb is 1, 2, 3 or 4;
ma+mb is 2, 3, 4, or 5;
R₁₄₁, R₁₄₂, R₁₄₃, R₁₄₄ and R₁₄₅ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
mc is 3;
three R₁₄₁ are mutually the same or different;
md is 3; and
three R₁₄₂ are mutually the same or different.

In the compound represented by the formula (1X), preferably, ma is 1 or 2 and mb is 1 or 2 in the formula (102X).

In the compound represented by the formula (1X), preferably, ma is 1 and mb is 1 in the formula (102X).

In the compound represented by the formula (1X), the group represented by the formula (11X) is also preferably a group represented by a formula (11AX) or a group represented by a formula (11BX) below.

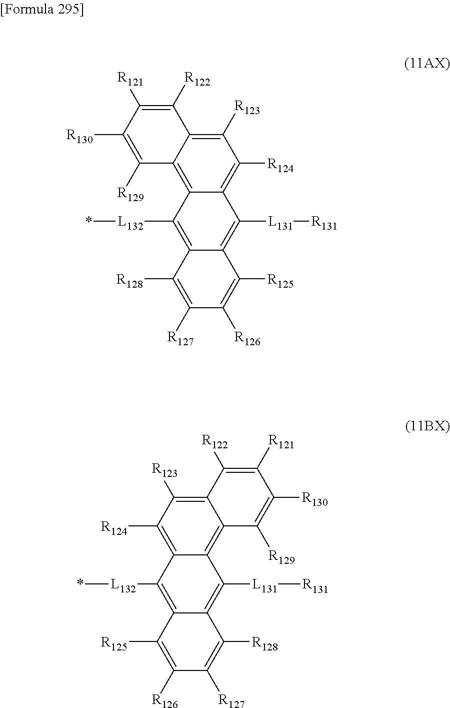

In the formulae (11AX) and (11BX):
R₁₂₁ to R₁₃₁ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
when a plurality of groups represented by the formula (11AX) are present, the plurality of groups represented by the formula (11AX) are mutually the same or different;
when a plurality of groups represented by the formula (11BX) are present, the plurality of groups represented by the formula (11BX) are mutually the same or different;
L₁₃₁ and L₁₃₂ are each independently a single bond, a substituted or unsubstituted arylene group having 6 to 50 ring carbon atoms, or a substituted or unsubstituted divalent heterocyclic group having 5 to 50 ring atoms; and
* in each of the formulae (11AX) and (11BX) represents a bonding position to a benz[a]anthracene ring in the formula (1X).

The compound represented by the formula (1X) is also preferably represented by a formula (103X) below.

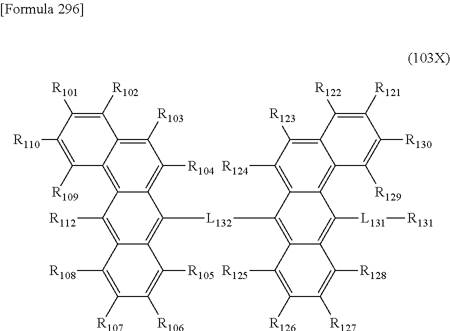

In the formula (103X):
R₁₀₁ to R₁₁₀ and R₁₁₂ respectively represent the same as R₁₀₁ to R₁₁₀ and R₁₁₂ in the formula (1X); and
R₁₂₁ to R₁₃₁, L₁₃₁, and L₁₃₂ respectively represent the same as R₁₂₁ to R₁₃₁, L₁₃₁, and L₁₃₂ in the formula (11 BX).

In the compound represented by the formula (1X), L₁₃₁ is also preferably a substituted or unsubstituted arylene group having 6 to 50 ring carbon atoms.

In the compound represented by the formula (1X), L₁₃₂ is also preferably a substituted or unsubstituted arylene group having 6 to 50 ring carbon atoms.

In the compound represented by the formula (1X), also preferably, two or more of R₁₀₁ to R₁₁₂ are each a group represented by the formula (11).

In the compound represented by the formula (1X), it is preferable that two or more of R₁₀₁ to R₁₁₂ are each a group represented by the formula (11X) and Ar₁₀₁ in the formula (11X) is a substituted or unsubstituted aryl group having 6 to 50 ring carbon atoms.

In the compound represented by the formula (1X), it is also preferable that Ar₁₀₁ is not a substituted or unsubstituted benz[a]anthryl group, L₁₀₁ is not a substituted or unsubstituted benz[a]anthrylene group, and
the substituted or unsubstituted aryl group having 6 to 50 ring carbon atoms for R₁₀₁ to R₁₁₀ not being the group represented by the formula (11X) is not a substituted or unsubstituted benz[a]anthryl group.

In the compound represented by the formula (1X), R₁₀₁ to R₁₁₂ not being the group represented by the formula (11X) are preferably each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

In the compound represented by the formula (1X), R₁₀₁ to R₁₁₂ not being the group represented by the formula (11X) are each preferably a hydrogen atom, a substituted or unsubstituted alkyl group having 1 to 50 carbon atoms, or a substituted or unsubstituted cycloalkyl group having 3 to 50 ring carbon atoms.

In the compound represented by the formula (1X), R₁₀₁ to R₁₁₂ not being the group represented by the formula (11X) are each preferably a hydrogen atom.

### Compound Represented by Formula (12X)

In an exemplary arrangement of the organic EL device according to the exemplary embodiment, the first compound is a compound represented by the formula (12X) below.

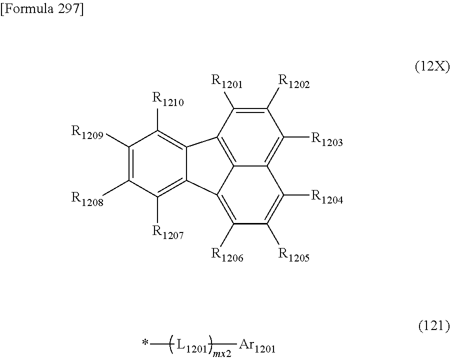

In the formula (12X):
at least one combination of adjacent two or more of R₁₂₀₁ to R₁₂₁₀ are mutually bonded to form a substituted or unsubstituted monocyclic ring, or mutually bonded to form a substituted or unsubstituted fused ring;
R₁₂₀₁ to R₁₂₁₀ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (121);
at least one of a substituent, if present, for the substituted or unsubstituted monocyclic ring, a substituent, if present, for the substituted or unsubstituted fused ring, or R₁₂₀₁ to R₁₂₁₀ is a group represented by the formula (121);
when a plurality of groups represented by the formula (121) are present, the plurality of groups represented by the formula (121) are mutually the same or different;
L₁₂₀₁ is a single bond, a substituted or unsubstituted arylene group having 6 to 50 ring carbon atoms, or a substituted or unsubstituted divalent heterocyclic group having 5 to 50 ring atoms;
Ar₁₂₀₁ is a substituted or unsubstituted aryl group having 6 to 50 ring carbon atoms, or a substituted or unsubstituted heterocyclic group having 5 to 50 ring atoms;
mx2 is 0, 1, 2, 3, 4, or 5;
when two or more L₁₂₀₁ are present, the two or more L₁₂₀₁ are mutually the same or different;
when two or more Ar₁₂₀₁ are present, the two or more Ar₁₂₀₁ are mutually the same or different; and
* in the formula (121) represents a bonding position to a ring represented by the formula (12X).

In the formula (12X), combinations of adjacent two of R₁₂₀₁ to R₁₂₁₀ refer to a combination of R₁₂₀₁ and R₁₂₀₂, a combination of R₁₂₀₂ and R₁₂₀₃, a combination of R₁₂₀₃ and R₁₂₀₄, a combination of R₁₂₀₄ and R₁₂₀₅, a combination of R₁₂₀₅ and R₁₂₀₆, a combination of R₁₂₀₇ and R₁₂₀₈, a combination of R₁₂₀₈ and R₁₂₀₉, and a combination of R₁₂₀₉ and R₁₂₁₀.

### Compound Represented by Formula (13X)

In an exemplary arrangement of the organic EL device according to the exemplary embodiment, the first compound is a compound represented by the formula (13X) below.

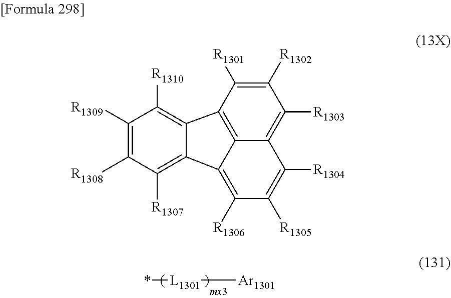

In the formula (13X):
R₁₃₀₁ to R₁₃₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (131);
at least one of R₁₃₀₁ to R₁₃₁₀ is a group represented by the formula (131);
when a plurality of groups represented by the formula (131) are present, the plurality of groups represented by the formula (131) are mutually the same or different;
L₁₃₀₁ is a single bond, a substituted or unsubstituted arylene group having 6 to 50 ring carbon atoms, or a substituted or unsubstituted divalent heterocyclic group having 5 to 50 ring atoms;
Ar₁₃₀₁ is a substituted or unsubstituted aryl group having 6 to 50 ring carbon atoms, or a substituted or unsubstituted heterocyclic group having 5 to 50 ring atoms;
mx3 is 0, 1, 2, 3, 4, or 5;
when two or more L₁₃₀₁ are present, the two or more L₁₃₀₁ are mutually the same or different;
when two or more Ar₁₃₀₁ are present, the two or more Ar₁₃₀₁ are mutually the same or different; and
* in the formula (131) represents a bonding position to a fluoranthene ring represented by the formula (13X).

In the organic EL device according to the exemplary embodiment, none of combinations of adjacent two or more of R₁₃₀₁ to R₁₃₁₀ not being the group represented by the formula (131) are bonded to each other. Combinations of adjacent two of R₁₃₀₁ to R₁₃₁₀ in the formula (13X) refer to a combination of R₁₃₀₁ and R₁₃₀₂, a combination of R₁₃₀₂ and R₁₃₀₃, a combination of R₁₃₀₃ and R₁₃₀₄, a combination of R₁₃₀₄ and R₁₃₀₅, a combination of R₁₃₀₅ and R₁₃₀₆, a combination of R₁₃₀₇ and R₁₃₀₈, a combination of R₁₃₀₈ and R₁₃₀₉, and a combination of R₁₃₀₉ and R₁₃₁₀.

### Compound Represented by Formula (14X)

In an exemplary arrangement of the organic EL device according to the exemplary embodiment, the first compound is a compound represented by the formula (14X) below.

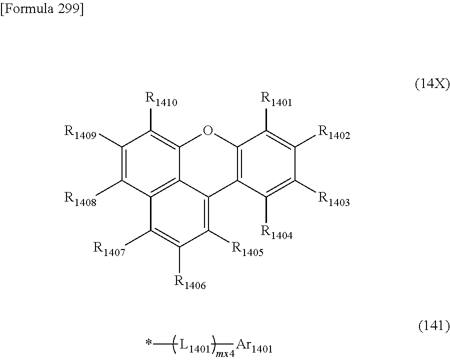

In the formula (14X):
R₁₄₀₁ to R₁₄₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (141);
at least one of R₁₄₀₁ to R₁₄₁₀ is a group represented by the formula (141);
when a plurality of groups represented by the formula (141) are present, the plurality of groups represented by the formula (141) are mutually the same or different;
L₁₄₀₁ is a single bond, a substituted or unsubstituted arylene group having 6 to 50 ring carbon atoms, or a substituted or unsubstituted divalent heterocyclic group having 5 to 50 ring atoms;
Ar₁₄₀₁ is a substituted or unsubstituted aryl group having 6 to 50 ring carbon atoms, or a substituted or unsubstituted heterocyclic group having 5 to 50 ring atoms;
mx4 is 0, 1, 2, 3, 4, or 5;
when two or more L₁₄₀₁ are present, the two or more L₁₄₀₁ are mutually the same or different;
when two or more Ar₁₄₀₁ are present, the two or more Ar₁₄₀₁ are mutually the same or different; and
* in the formula (141) represents a bonding position to a ring represented by the formula (14X).

### Compound Represented by Formula (15X)

In an exemplary arrangement of the organic EL device according to the exemplary embodiment, the first compound is a compound represented by the formula (15X) below.

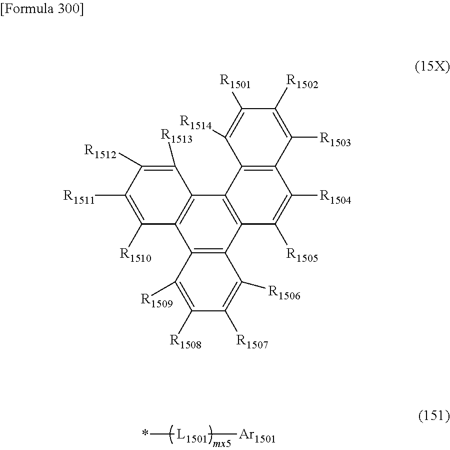

In the formula (15X):
R₁₅₀₁ to R₁₅₁₄ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (151);
at least one of R₁₅₀₁ to R₁₅₁₄ is a group represented by the formula (151);
when a plurality of groups represented by the formula (151) are present, the plurality of groups represented by the formula (151) are mutually the same or different;
L₁₅₀₁ is a single bond, a substituted or unsubstituted arylene group having 6 to 50 ring carbon atoms, or a substituted or unsubstituted divalent heterocyclic group having 5 to 50 ring atoms;
Ar₁₅₀₁ is a substituted or unsubstituted aryl group having 6 to 50 ring carbon atoms, or a substituted or unsubstituted heterocyclic group having 5 to 50 ring atoms;
mx5 is 0, 1, 2, 3, 4, or 5;
when two or more L₁₅₀₁ are present, the two or more L₁₅₀₁ are mutually the same or different;
when two or more Ar₁₅₀₁ are present, the two or more Ar₁₅₀₁ are mutually the same or different; and
* in the formula (151) represents a bonding position to a ring represented by the formula (15X).

### Compound Represented by Formula (16X)

In an exemplary arrangement of the organic EL device according to the exemplary embodiment, the first compound is a compound represented by the formula (16X) below.

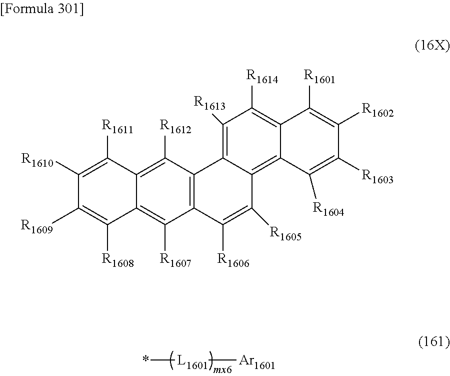

In the formula (16X):
R₁₆₀₁ to R₁₆₁₄ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (161);
at least one of R₁₆₀₁ to R₁₆₁₄ is a group represented by the formula (161);
when a plurality of groups represented by the formula (161) are present, the plurality of groups represented by the formula (161) are mutually the same or different;
L₁₆₀₁ is a single bond, a substituted or unsubstituted arylene group having 6 to 50 ring carbon atoms, or a substituted or unsubstituted divalent heterocyclic group having 5 to 50 ring atoms;
Ar₁₆₀₁ is a substituted or unsubstituted aryl group having 6 to 50 ring carbon atoms, or a substituted or unsubstituted heterocyclic group having 5 to 50 ring atoms;
mx6 is 0, 1, 2, 3, 4, or 5;
when two or more L₁₆₀₁ are present, the two or more L₁₆₀₁ are mutually the same or different;
when two or more Ar₁₆₀₁ are present, the two or more Ar₁₆₀₁ are mutually the same or different; and
* in the formula (161) represents a bonding position to a ring represented by the formula (16X).

### Compound Represented by Formula (1000B)

In an exemplary arrangement of the organic EL device according to the exemplary embodiment, the first compound is a compound represented by the formula (1000B) below.

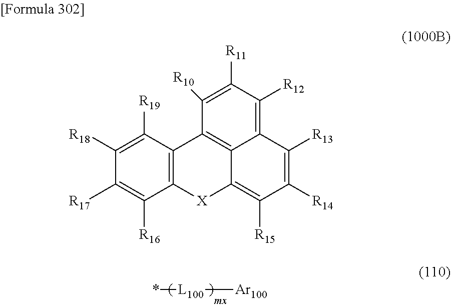

In the formula (1000B):
X is an oxygen atom or a sulfur atom;
at least one combination of adjacent two or more of R₁₀ to R₁₉ are mutually bonded to form a substituted or unsubstituted monocyclic ring, mutually bonded to form a substituted or unsubstituted fused ring, or not mutually bonded;
R₁₀ to R₁₉ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a nitro group, a substituted or unsubstituted aryl group having 6 to 50 ring carbon atoms, a substituted or unsubstituted heterocyclic group having 5 to 50 ring atoms, or a group represented by the formula (110);
at least one of R₁₀ to R₁₉ is a group represented by the formula (110);
when a plurality of groups represented by the formula (110) are present, the plurality of groups represented by the formula (110) are mutually the same or different;
L₁₀₀ is a single bond, a substituted or unsubstituted arylene group having 6 to 50 ring carbon atoms, or a substituted or unsubstituted divalent heterocyclic group having 5 to 50 ring atoms;
mx is 1, 2, or 3;
when two or more L₁₀₀ are present, the two or more L₁₀₀ are mutually the same or different;
Ar₁₀₀ is a substituted or unsubstituted aryl group having three or more rings, or a substituted or unsubstituted heterocyclic group having two or more aromatic rings and one or more heterocycles;
Ar₁₀₀ contains no anthracene ring;
when two or more Ar₁₀₀ are present, the two or more Ar₁₀₀ are mutually the same or different; and
* in the formula (110) represents a bonding position;
in the first compound represented by the formula (1000B), R₉₀₁, R₉₀₂, R₉₀₃, R₉₀₄, R₉₀₅, R₉₀₆, R₉₀₇, R₈₀₁ and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different;
when a plurality of R₉₀₇ are present, the plurality of R₉₀₇ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

In the formula (1000B), X is preferably an oxygen atom.

The compound represented by the formula (1000B) is preferably a compound that has at least one group represented by the formula (110) and is represented by a formula (100) below.

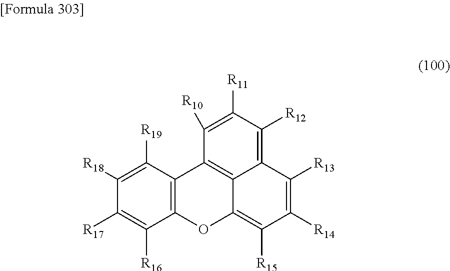

In the formula (100): R₁₀ to R₁₉ each independently represent the same as R₁₀ to R₁₉ in the formula (1000B); and Ar₁₀₀, L₁₀₀ and mx respectively represent the same as Ar₁₀₀, L₁₀₀ and mx in the formula (110).

The compound represented by the formula (1000B) is also preferably a compound represented by a formula (101) or (102) below.

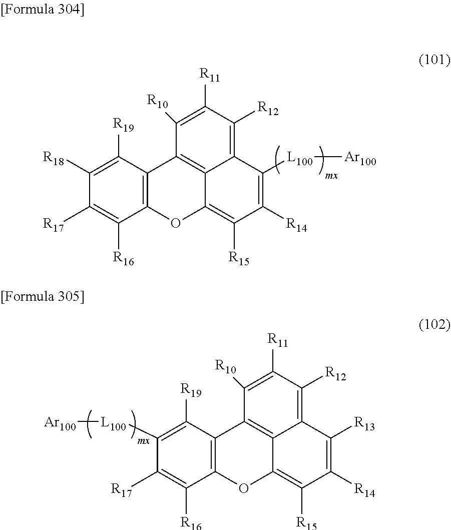

In the formulae (101) and (102): R₁₀ to R₁₉ each independently represent the same as R₁₀ to R₁₉ in the formula (1000B); and Ar₁₀₀, L₁₀₀ and mx respectively represent the same as Ar₁₀₀, L₁₀₀ and mx in the formula (110).

In the formula (1000B), R₁₀ to R₁₉ not being the group represented by the formula (110) are preferably each independently a hydrogen atom, a substituted or unsubstituted alkyl group having 1 to 50 carbon atoms, a substituted or unsubstituted cycloalkyl group having 3 to 50 ring carbon atoms, or a substituted or unsubstituted aryl group having 6 to 50 ring carbon atoms.

In the formula (1000B), R₁₀ to R₁₉ not being the group represented by the formula (110) are each preferably a hydrogen atom.

In the formula (1000B), L₁₀₀ is preferably a single bond or an arylene group having three or less substituted or unsubstituted benzene rings.

In the formula (1000B), L₁₀₀ is preferably not a substituted or unsubstituted anthrylene group.

In the formula (1000B), L₁₀₀ is also preferably a single bond.

In the formula (1000B), the group represented by -(L₁₀₀)ₘₓ- in the formula (110) is also preferably a group represented by any one of formulae (111) to (120) below.

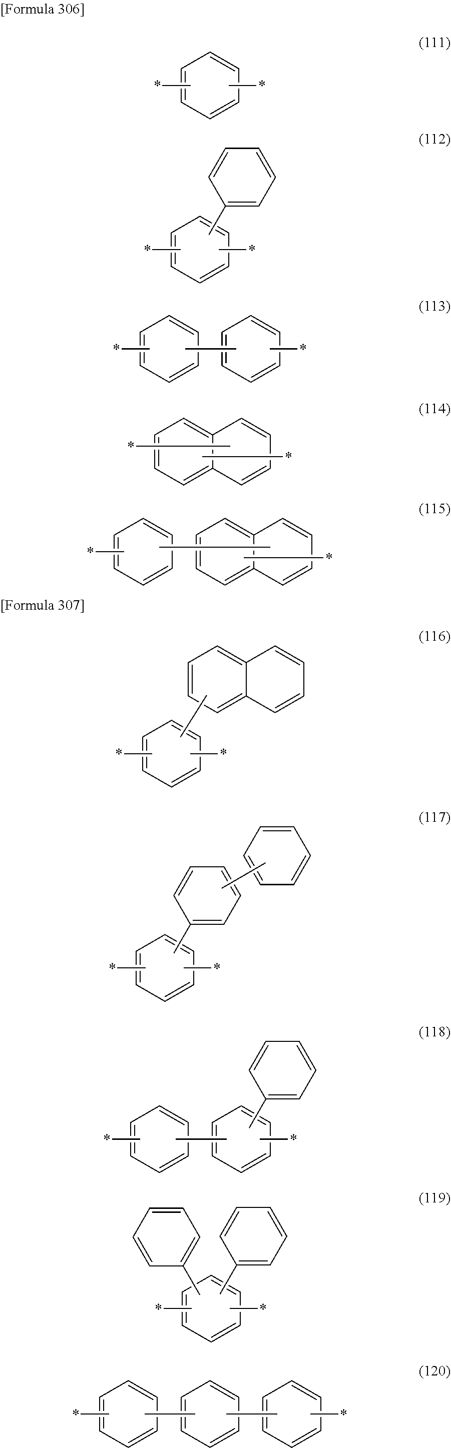

In the formulae (111) to (120), * each represent a bonding position.

The group represented by -(L₁₀₀)ₘₓ- in the formula (110) is preferably a group represented by the formula (111) or (112).

In the formula (1000B), Ar₁₀₀ is preferably an aryl group in which at least four substituted or unsubstituted benzene rings are fused.

In the formula (1000B), Ar₁₀₀ is preferably an aryl group in which four substituted or unsubstituted benzene rings are fused or an aryl group in which five substituted or unsubstituted benzene rings are fused.

In the formula (1000B): Ar₁₀₀ is preferably a group represented by a formula (1100), a formula (1200), a formula (1300), a formula (1400), a formula (1500), a formula (1600), a formula (1700) or a formula (1800) below.

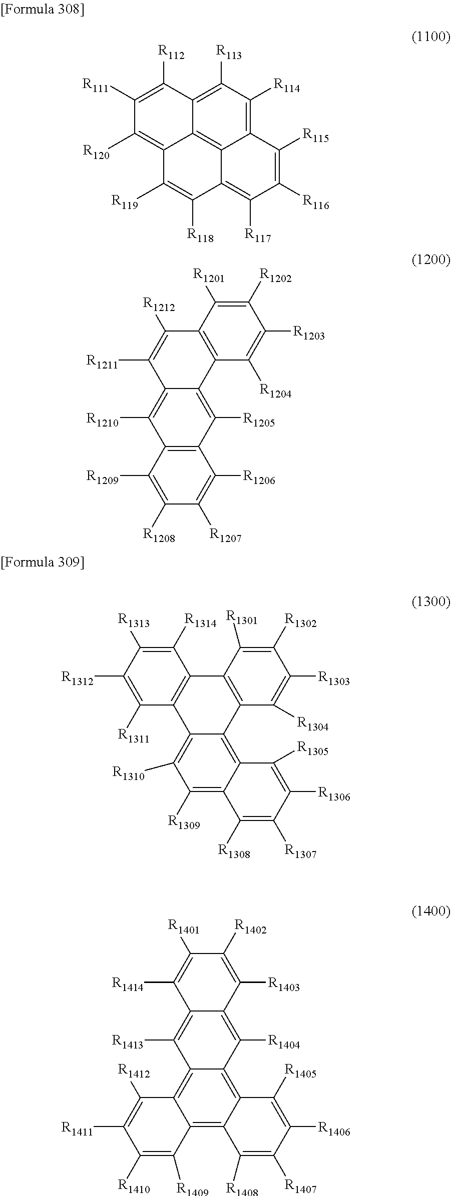

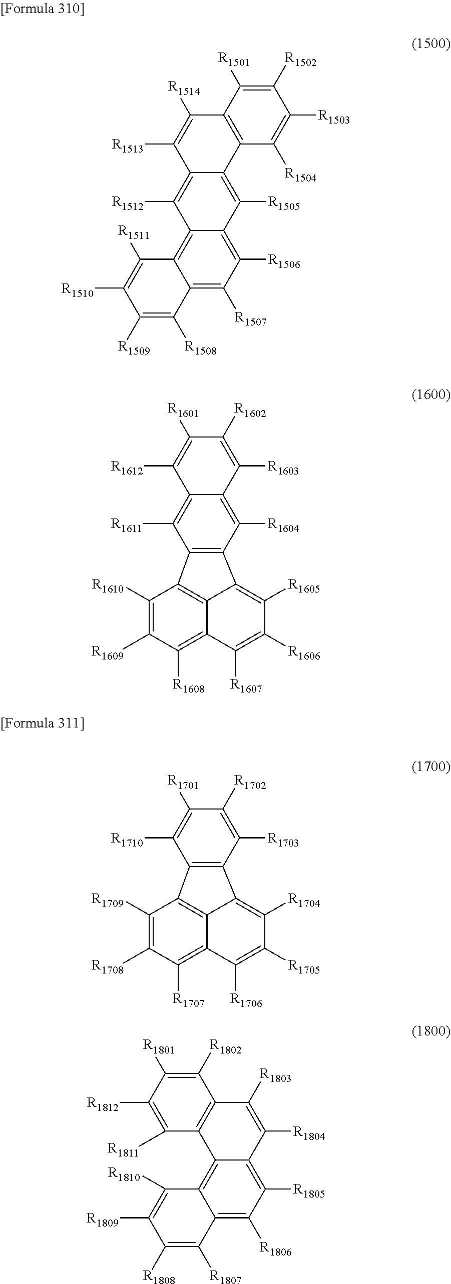

In the formula (1100), one of R₁₁₁ to R₁₂₀ is a bond;
in the formula (1200), one of R₁₂₀₁ to R₁₂₁₂ is a bond;
in the formula (1300), one of R₁₃₀₁ to R₁₃₁₄ is a bond;
in the formula (1400), one of R₁₄₀₁ to R₁₄₁₄ is a bond;
in the formula (1500), one of R₁₅₀₁ to R₁₅₁₄ is a bond;
in the formula (1600), one of R₁₆₀₁ to R₁₆₁₂ is a bond;
in the formula (1700), one of R₁₇₀₁ to R₁₇₁₀ is a bond; and
in the formula (1800), one of R₁₈₀₁ to R₁₈₁₂ is a bond; and
R₁₁₁ to R₁₂₀ not being the bond, R₁₂₀₁ to R₁₂₁₂ not being the bond, R₁₃₀₁ to R₁₃₁₄ not being the bond, R₁₄₀₁ to R₁₄₁₄ not being the bond, R₁₅₀₁ to R₁₅₁₄ not being the bond, R₁₆₀₁ to R₁₆₁₂ not being the bond, R₁₇₀₁ to R₁₇₁₀ not being the bond, and R₁₈₀₁ to R₁₈₁₂ not being the bond are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a nitro group, a substituted or unsubstituted aryl group having 6 to 50 ring carbon atoms, or a substituted or unsubstituted heterocyclic group having 5 to 50 ring atoms.

The group represented by the formula (1100) in which R₁₁₁ is the bond is represented by a formula (1112) below. The group represented by the formula (1100) in which R₁₂₀ is the bond is represented by a formula (1113) below. The group represented by the formula (1100) in which R₁₁₉ is the bond is represented by a formula (1114) below.

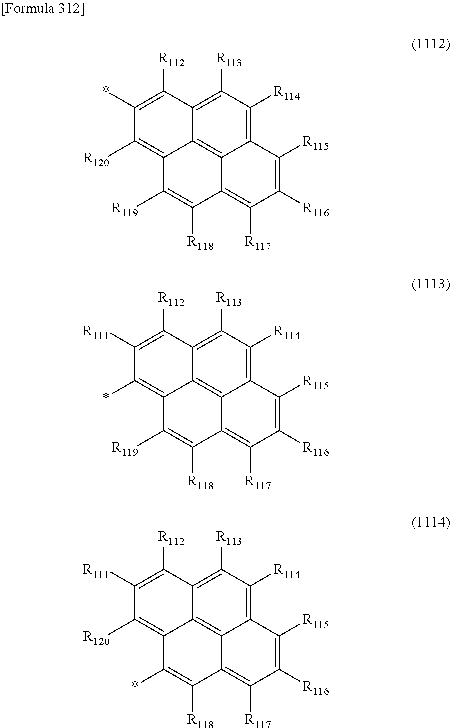

In the formulae (1112), (1113), and (1114):
R₁₁₁ to R₁₂₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃),
a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a nitro group, a substituted or unsubstituted aryl group having 6 to 50 ring carbon atoms, or a substituted or unsubstituted heterocyclic group having 5 to 50 ring atoms; and
* in the formulae (1112) to (1114) each represent a bonding position.

In the formulae (1100), (1200), (1300), (1400), (1500), (1600), (1700) and (1800), R₁₁₁ to R₁₂₀ not being the bond, R₁₂₀₁ to R₁₂₁₂ not being the bond, R₁₃₀₁ to R₁₃₁₄ not being the bond, R₁₄₀₁ to R₁₄₁₄ not being the bond, R₁₅₀₁ to R₁₅₁₄ not being the bond, R₁₆₀₁ to R₁₆₁₂ not being the bond, R₁₇₀₁ to R₁₇₁₀ not being the bond, and R₁₈₀₁ to R₁₈₁₂ not being the bond are preferably each independently a hydrogen atom, a substituted or unsubstituted alkyl group having 1 to 50 carbon atoms, a substituted or unsubstituted cycloalkyl group having 3 to 50 ring carbon atoms, or a substituted or unsubstituted aryl group having 6 to 50 ring carbon atoms.

In the formulae (1100), (1200), (1300), (1400), (1500), (1600), (1700) and (1800), R₁₁₁ to R₁₂₀ not being the bond, R₁₂₀₁ to R₁₂₁₂ not being the bond, R₁₃₀₁ to R₁₃₁₄ not being the bond, R₁₄₀₁ to R₁₄₁₄ not being the bond, R₁₅₀₁ to R₁₅₁₄ not being the bond, R₁₆₀₁ to R₁₆₁₂ not being the bond, R₁₇₀₁ to R₁₇₁₀ not being the bond, and R₁₈₀₁ to R₁₈₁₂ not being the bond are preferably each a hydrogen atom.

The compound represented by the formula (1000B) preferably includes only one benzoxanthene ring in a molecule.

The compounds represented by the formulae (100), (101) and (102) in which a benzoxanthene ring is substituted by a benzothioxanthene ring are also preferable.

### Compound Represented by Formula (17X-1)

In an exemplary arrangement of the organic EL device according to the exemplary embodiment, the first compound is a compound represented by the formula (17X-1) below.

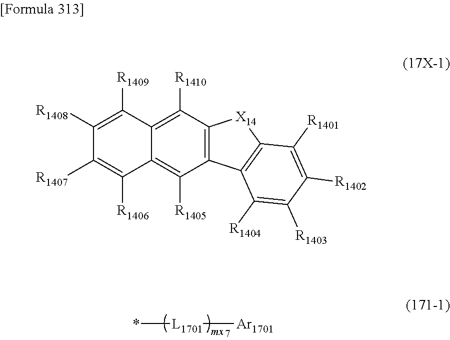

In the formula (17X-1):
X₁₄ is an oxygen atom or a sulfur atom;
at least one combination of adjacent two or more of R₁₄₀₁ to R₁₄₀₄ are mutually bonded to form a substituted or unsubstituted monocyclic ring, mutually bonded to form a substituted or unsubstituted fused ring, or not mutually bonded;
at least one combination of adjacent two or more of R₁₄₀₅ to R₁₄₁₀ are mutually bonded to form a substituted or unsubstituted monocyclic ring, mutually bonded to form a substituted or unsubstituted fused ring, or not mutually bonded;
at least one of R₁₄₀₁ to R₁₄₁₀ is a group represented by the formula (171-1);
R₁₄₀₁ to R₁₄₁₀ forming neither the monocyclic ring nor the fused ring and not being the group represented by the formula (171-1) are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (171-1);
at least one of R₁₄₀₁ to R₁₄₁₀ is a group represented by the formula (171-1);
when a plurality of groups represented by the formula (171-1) are present, the plurality of groups represented by the formula (171-1) are mutually the same or different;
L₁₇₀₁ is a single bond, a substituted or unsubstituted arylene group having 6 to 50 ring carbon atoms, or a substituted or unsubstituted divalent heterocyclic group having 5 to 50 ring atoms;
A₁₇₀₁ is a substituted or unsubstituted aryl group having 6 to 50 ring carbon atoms, or a substituted or unsubstituted heterocyclic group having 5 to 50 ring atoms;
mx7 is 0, 1, 2, 3, 4, or 5;
when two or more L₁₇₀₁ are present, the two or more L₁₇₀₁ are mutually the same or different;
when two or more Ar₁₇₀₁ are present, the two or more Ar₁₇₀₁ are mutually the same or different;
R₉₀₁ to R₉₀₅, R₈₀₁ and R₈₀₂ each independently represent the same as R₉₀₁ to R₉₀₅, R₈₀₁ and R₈₀₂ in the formula (1000B); and
* in the formula (171-1) represents a bonding position to a ring represented by the formula (17X-1).

### Compound Represented by Formula (17X-2)

In an exemplary arrangement of the organic EL device according to the exemplary embodiment, the first compound is a compound represented by the formula (17X-2) below.

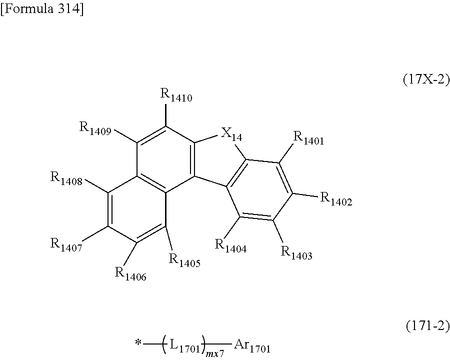

In the formula (17X-2), R₁₄₀₁ to R₁₄₁₀ and X₁₄ each independently represent the same as R₁₄₀₁ to R₁₄₁₀ and X₁₄ in the formula (17X-1);
the group represented by the formula (171-2) represents the same as a group represented by the formula (171-1); and L₁₇₀₁, Ar₁₇₀₁ and mx7 in the formula (171-2) each independently represent the same as L₁₇₀₁, Ar₁₇₀₁ and mx7 in the formula (171-1);
when a plurality of groups represented by the formula (171-2) are present, the plurality of groups represented by the formula (171-2) are mutually the same or different; and
* in the formula (171-2) represents a bonding position to a ring represented by the formula (17X-2).

### Compound Represented by Formula (17X-3)

In an exemplary arrangement of the organic EL device according to the exemplary embodiment, the first compound is a compound represented by the formula (17X-3) below.

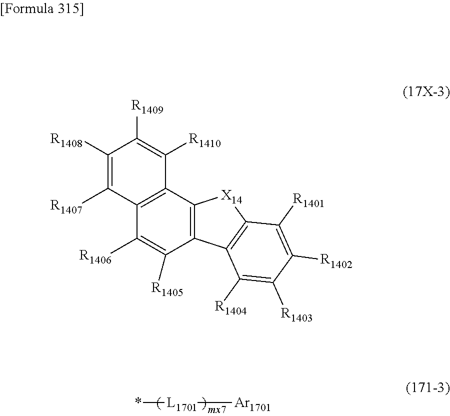

In the formula (17X-3), R₁₄₀₁ to R₁₄₁₀ and X₁₄ each independently represent the same as R₁₄₀₁ to R₁₄₁₀ and X₁₄ in the formula (17X-1);
the group represented by the formula (171-3) represents the same as a group represented by the formula (171-1); and L₁₇₀₁, Ar₁₇₀₁ and mx7 in the formula (171-3) each independently represent the same as L₁₇₀₁, Ar₁₇₀₁ and mx7 in the formula (171-1);
when a plurality of groups represented by the formula (171-3) are present, the plurality of groups represented by the formula (171-3) are mutually the same or different; and
* in the formula (171-3) represents a bonding position to a ring represented by the formula (17X-3).

In the formulae (17X-1), (17X-2) and (17X-3), X₁₄ is preferably an oxygen atom.

### Compound Represented by Formula (18)

In an exemplary arrangement of the organic EL device according to the exemplary embodiment, the first compound is a compound represented by the formula (18) below.

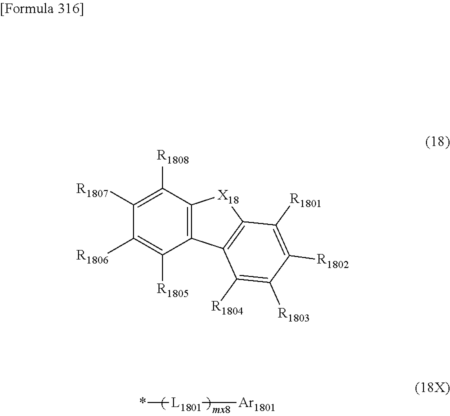

In the formula (18):
X₁₈ is an oxygen atom or a sulfur atom;
at least one combination of adjacent two or more of R₁₈₀₁ to R₁₈₀₄ are mutually bonded to form a substituted or unsubstituted monocyclic ring, mutually bonded to form a substituted or unsubstituted fused ring, or not mutually bonded;
at least one combination of adjacent two or more of R₁₈₀₅ to R₁₈₀₈ are mutually bonded to form a substituted or unsubstituted monocyclic ring, mutually bonded to form a substituted or unsubstituted fused ring, or not mutually bonded;
at least one of R₁₈₀₁ to R₁₈₀₈ is a group represented by the formula (18X);
R₁₈₀₁ to R₁₈₀₈ forming neither the monocyclic ring nor the fused ring and not being the group represented by the formula (18X) are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (18X);
at least one of R₁₈₀₁ to R₁₈₀₈ is a group represented by the formula (18X);
when a plurality of groups represented by the formula (18X) are present, the plurality of groups represented by the formula (18X) are mutually the same or different;
L₁₈₀₁ is a single bond, a substituted or unsubstituted arylene group having 6 to 50 ring carbon atoms, or a substituted or unsubstituted divalent heterocyclic group having 5 to 50 ring atoms;
A₁₈₀₁ is a substituted or unsubstituted aryl group having 6 to 50 ring carbon atoms, or a substituted or unsubstituted heterocyclic group having 5 to 50 ring atoms;
mx8 is 0, 1, 2, 3, 4, or 5;
when two or more L₁₈₀₁ are present, the two or more L₁₈₀₁ are mutually the same or different;
when two or more Ar₁₈₀₁ are present, the two or more Ar₁₈₀₁ are mutually the same or different;
R₉₀₁ to R₉₀₅, R₈₀₁ and R₈₀₂ each independently represent the same as R₉₀₁ to R₉₀₅, R₈₀₁ and R₈₀₂ in the formula (1000B); and
* in the formula (18X) represents a bonding position to a ring represented by the formula (18).

In the formula (18), X₁₈ is preferably an oxygen atom.

In the first compound, the groups specified to be “substituted or unsubstituted” are each preferably an “unsubstituted” group.

In the organic EL device according to the exemplary embodiment, it is also preferable that the second host material has, in a molecule, a linking structure including a benzene ring and a naphthalene ring that are linked with a single bond, in which the benzene ring and the naphthalene ring in the linking structure are each independently fused or not fused with a further monocyclic ring or fused ring, and the benzene ring and the naphthalene ring in the linking structure are further linked to each other by cross-linking at at least one site other than the single bond.

When the second host material has the linking structure including such cross-linking, deterioration in chromaticity of the organic EL device is expected to be inhibited.

The second host material in the above case is only required to have a linking structure as the minimum unit in a molecule, the linking structure including a benzene ring and a naphthalene ring linked to each other with a single bond (occasionally referred to as a benzene-naphthalene linking structure), the linking structure being as represented by a formula (X1) or a formula (X2) below. The benzene ring may be fused with a further monocyclic ring or fused ring, and the naphthalene ring may be fused with a further monocyclic ring or fused ring. For instance, also in a case where the second host material has, in a molecule, a linking structure including a naphthalene ring and a naphthalene ring linked to each other with a single bond (occasionally referred to as a naphthalene-naphthalene linking structure) and being as represented by a formula (X3), a formula (X4), or a formula (X5) below, the naphthalene-naphthalene linking structure is regarded as including the benzene-naphthalene linking structure since one of the naphthalene rings includes a benzene ring.

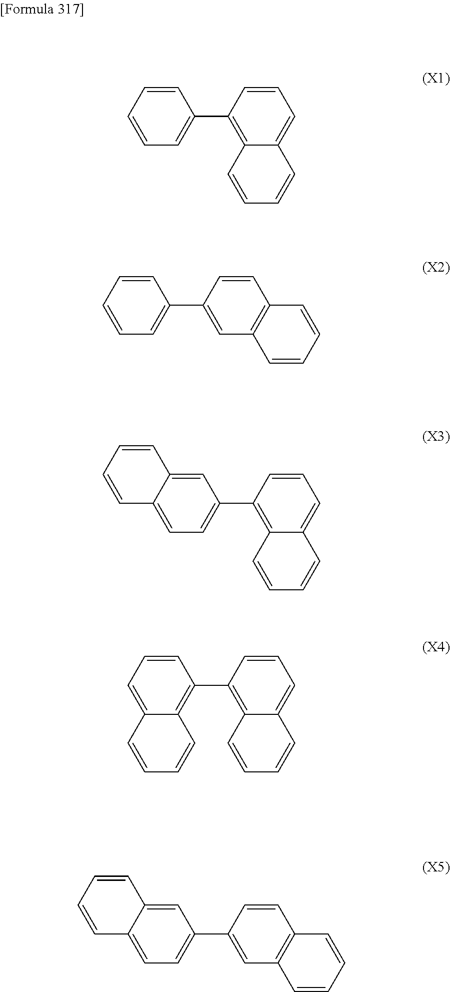

In the organic EL device according to the exemplary embodiment, the cross-linking also preferably includes a double bond. Specifically, the second host material also preferably has a structure in which the benzene ring and the naphthalene ring are further linked to each other at any other site than the single bond by the cross-linking structure including a double bond.

Assuming that the benzene ring and the naphthalene ring in the benzene-naphthalene linking structure are further linked to each other at at least one site other than the single bond by cross-linking, for instance, a linking structure (fused ring) represented by a formula (X11) below is obtained in a case of the formula (X1), and a linking structure (fused ring) represented by a formula (X31) below is obtained in a case of the formula (X3).

Assuming that the benzene ring and the naphthalene ring in the benzene-naphthalene linking structure are further linked to each other at any other site than the single bond by cross-linking including a double bond, for instance, a linking structure (fused ring) represented by a formula (X12) below is obtained in a case of the formula (X1), a linking structure (fused ring) represented by a formula (X21) or formula (X22) below is obtained in a case of the formula (X2), a linking structure (fused ring) represented by a formula (X41) below is obtained in a case of the formula (X4), and a linking structure (fused ring) represented by a formula (X51) below is obtained in a case of the formula (X5).

Assuming that the benzene ring and the naphthalene ring in the benzene-naphthalene linking structure are further linked to each other at at least one site other than the single bond by cross-linking including a hetero atom (e.g., an oxygen atom), for instance, a linking structure (fused ring) represented by a formula (X13) below is obtained in a case of the formula (X1).

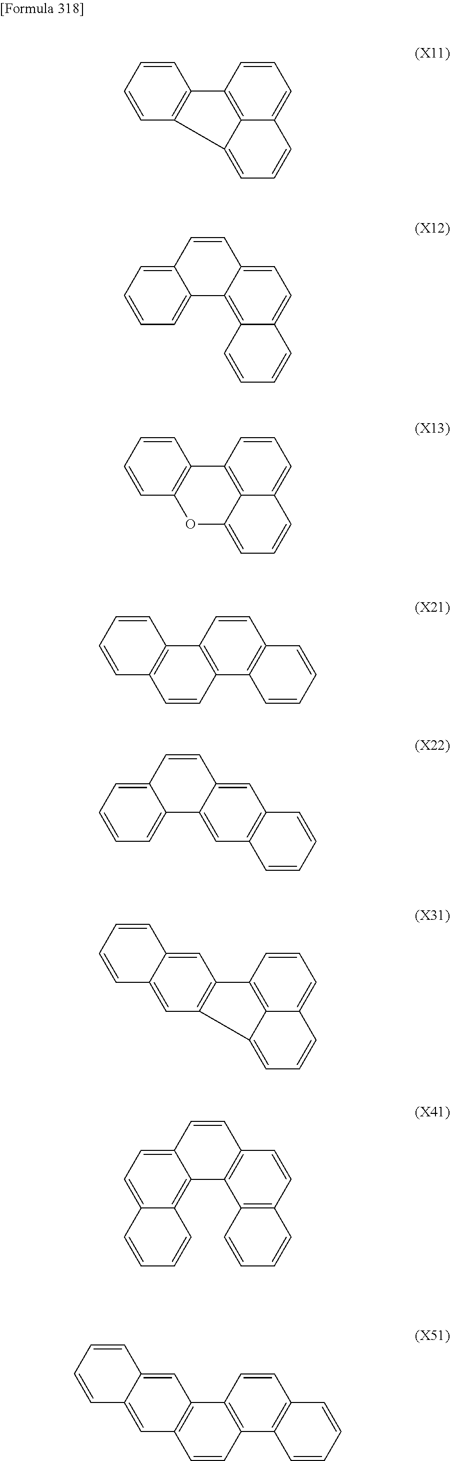

In the organic EL device according to the exemplary embodiment, also preferably, the second host material has, in a molecule, a biphenyl structure including a first benzene ring and a second benzene ring linked to each other with a single bond, and the first benzene ring and the second benzene ring in the biphenyl structure are further linked to each other by cross-linking at at least one site other than the single bond.

In the organic EL device according to the above exemplary embodiment, also preferably, the first benzene ring and the second benzene ring in the biphenyl structure are further linked to each other by the cross-linking at one site other than the single bond. When the second host material has the biphenyl structure including such cross-linking, deterioration in chromaticity of the organic EL device is expected to be inhibited.

In the organic EL device according to the above exemplary embodiment, the cross-linking also preferably includes a double bond.

In the organic EL device according to the above exemplary embodiment, the cross-linking also preferably includes no double bond.

Also preferably, the first benzene ring and the second benzene ring in the biphenyl structure are further linked to each other by the cross-linking at two sites other than the single bond.

In the organic EL device according to the exemplary embodiment, also preferably, the first benzene ring and the second benzene ring in the biphenyl structure are further linked to each other by the cross-linking at two sites other than the single bond, and the cross-linking includes no double bond. When the second host material has the biphenyl structure including such cross-linking, deterioration in chromaticity of the organic EL device is expected to be inhibited.

For instance, assuming that the first benzene ring and the second benzene ring in the biphenyl structure represented by a formula (BP1) below are further linked to each other by cross-linking at at least one site other than the single bond, the biphenyl structure is exemplified by linking structures (fused rings) represented by formulae (BP11) to (BP15) below.

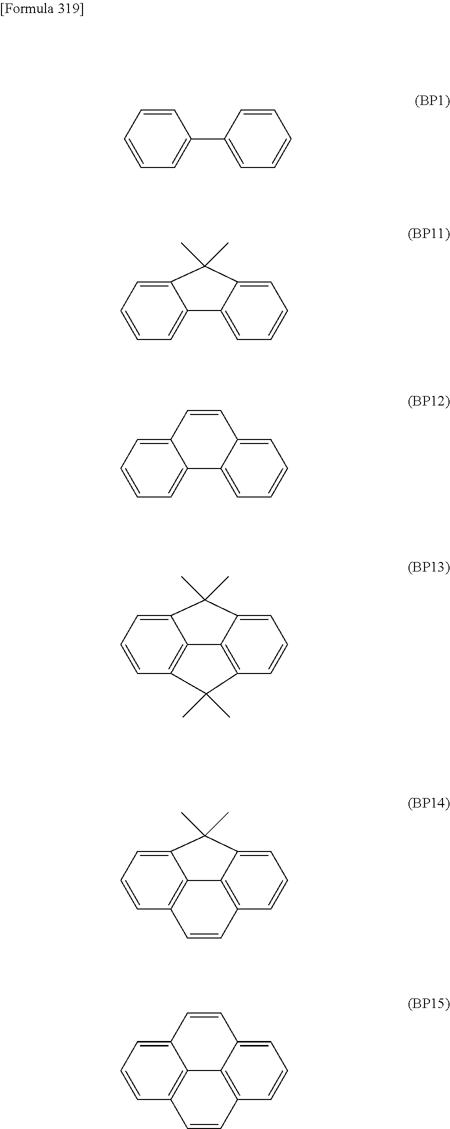

The formula (BP11) represents a linking structure in which the first benzene ring and the second benzene ring are linked to each other at one site other than the single bond by cross-linking including no double bond.

The formula (BP12) represents a linking structure in which the first benzene ring and the second benzene ring are linked to each other at one site other than the single bond by cross-linking including a double bond.

The formula (BP13) represents a linking structure in which the first benzene ring and the second benzene ring are linked to each other at two sites other than the single bond by cross-linking including no double bond.

The formula (BP14) represents a linking structure in which the first benzene ring and the second benzene ring are linked to each other by cross-linking including no double bond at one of two sites other than the single bond, and the first benzene ring and the second benzene ring are linked to each other by cross-linking including a double bond at the other of the two sites other than the single bond.

The formula (BP15) represents a linking structure in which the first benzene ring and the second benzene ring are linked to each other at two sites other than the single bond by cross-linking including a double bond.

In the first compound, the groups specified to be “substituted or unsubstituted” are each preferably an “unsubstituted” group.

### Method of Producing First Compound

The first compound usable in the organic EL device according to the exemplary embodiment can be produced by a known method. The first compound can also be produced based on a known method through a known alternative reaction using a known material(s) tailored for the target compound.

### Specific Examples of First Compound

Specific examples of the first compound usable in the organic EL device according to the exemplary embodiment include the following compounds. It should however be noted that the invention is not limited to the specific examples of the first compound.

In the specific examples of the compound herein, D represents a deuterium atom, Me represents a methyl group, and tBu represents a tert-butyl group.

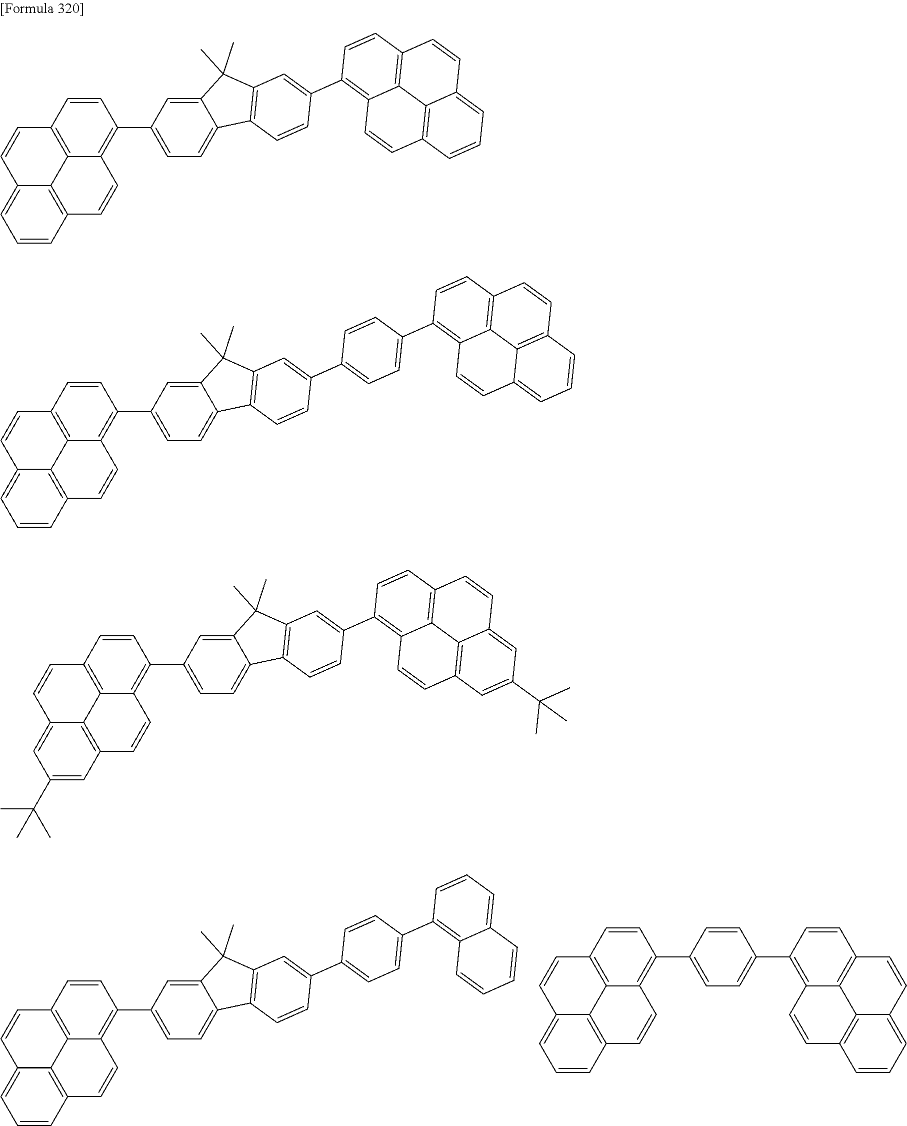

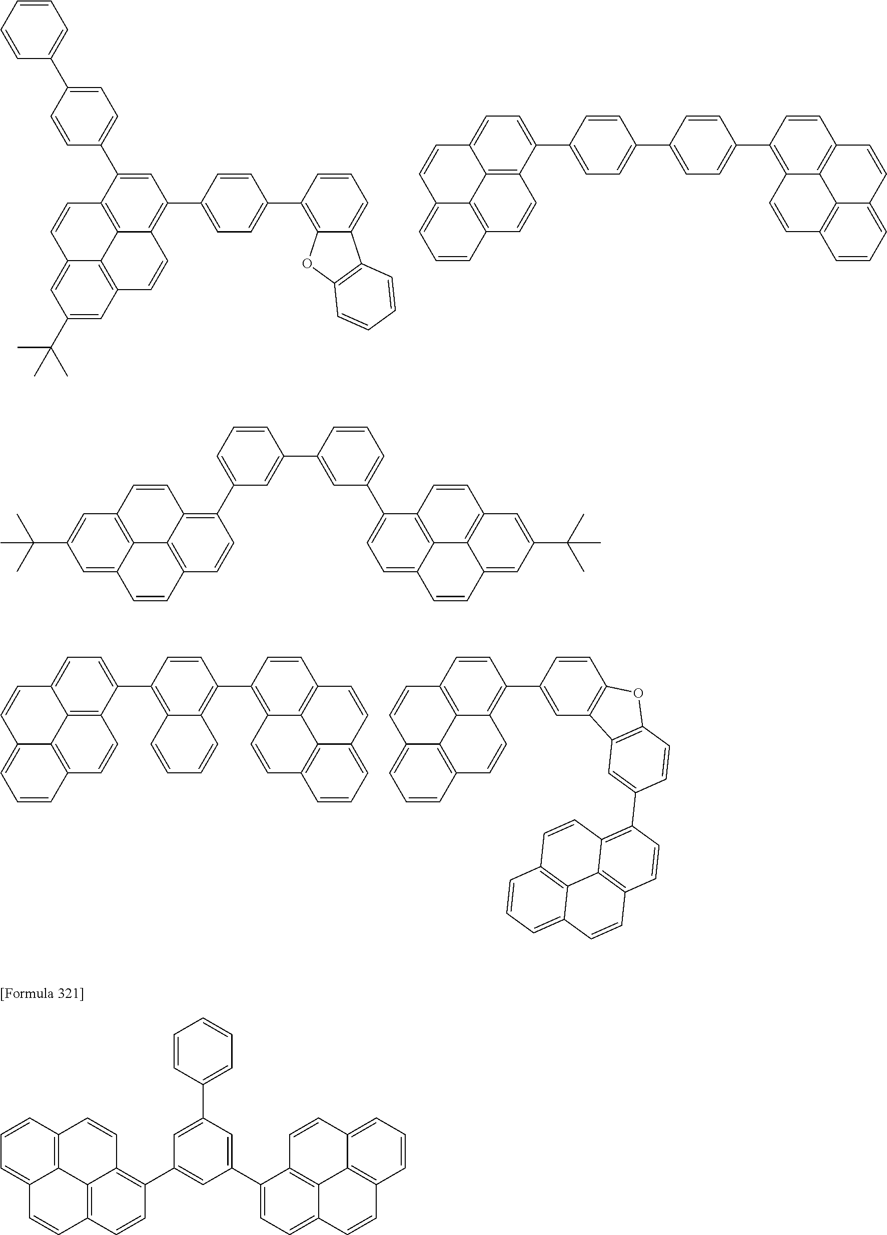

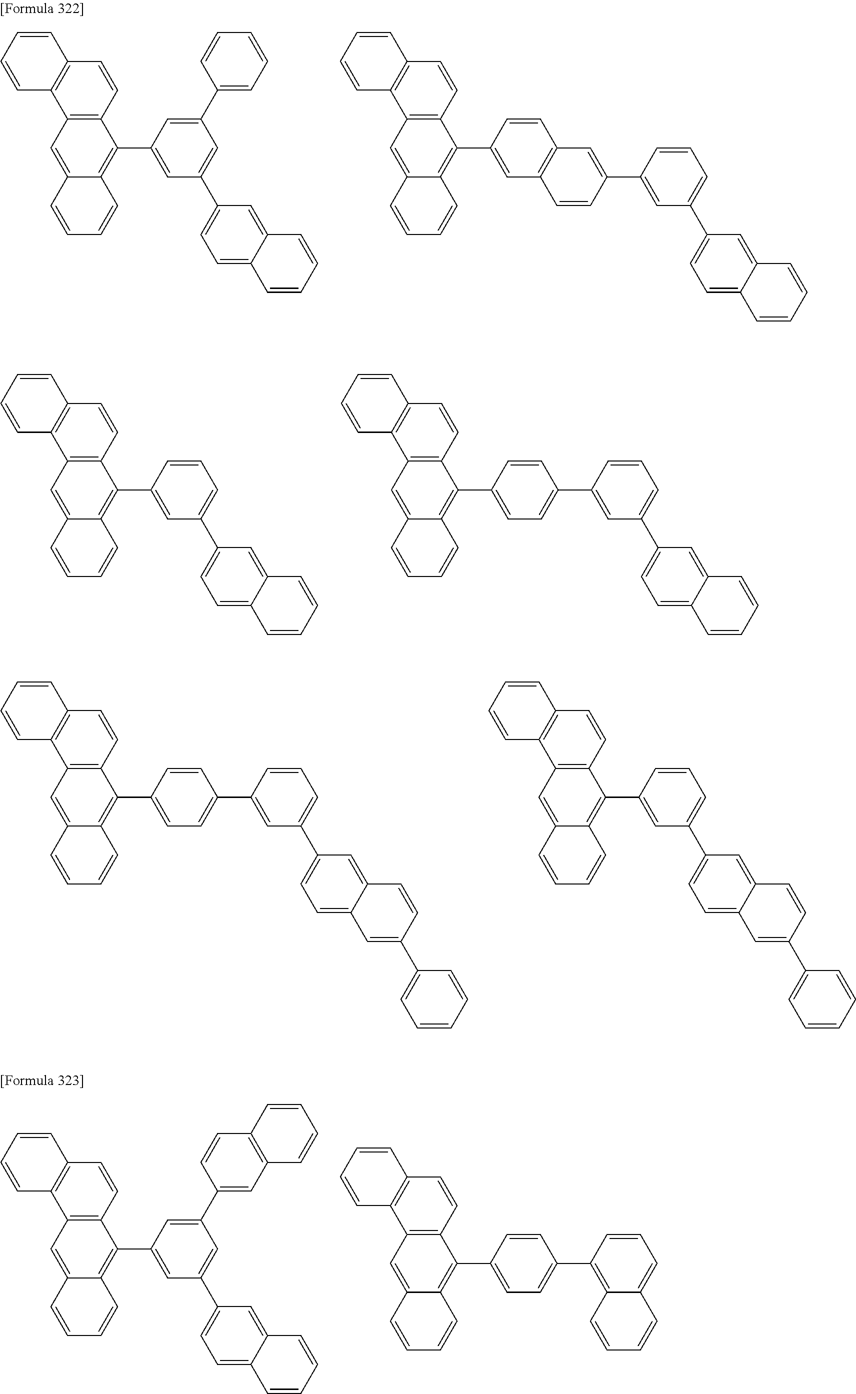

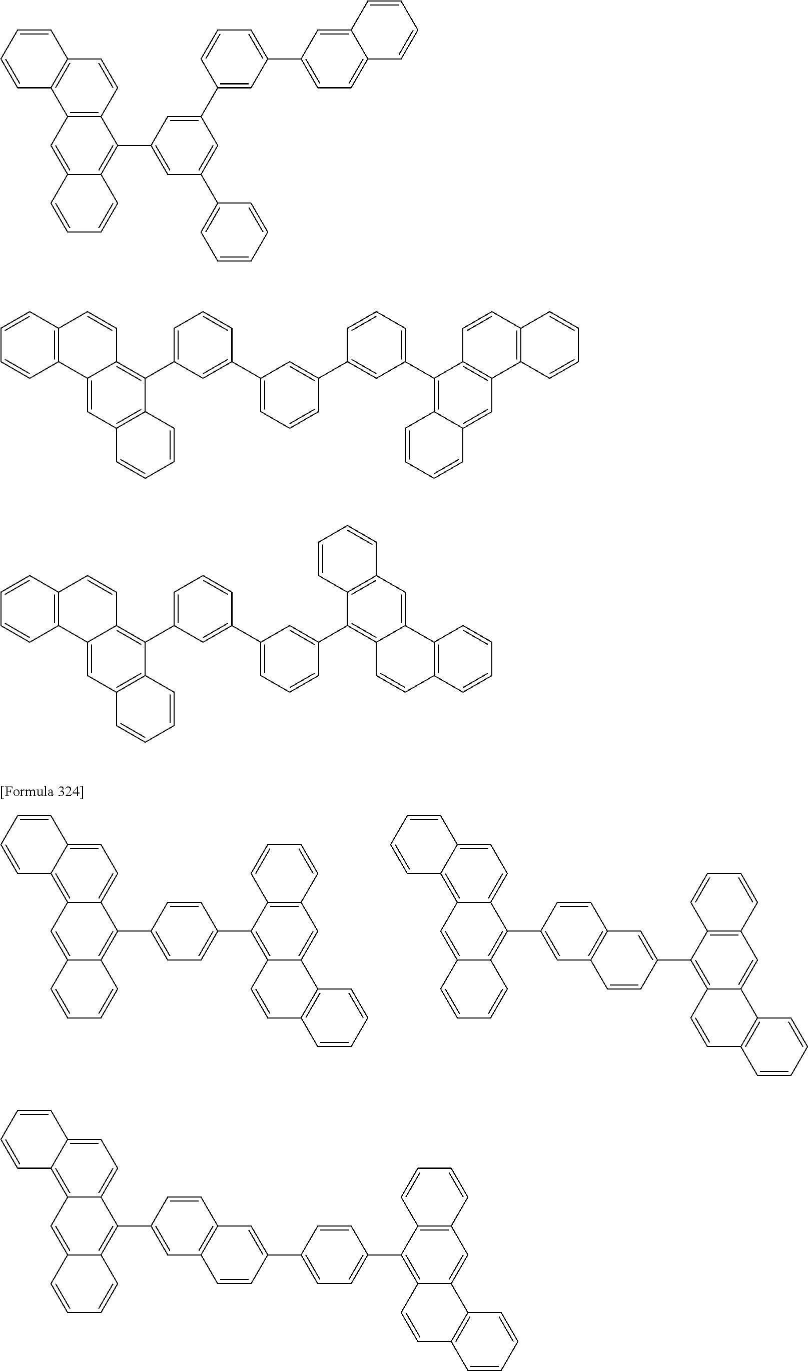

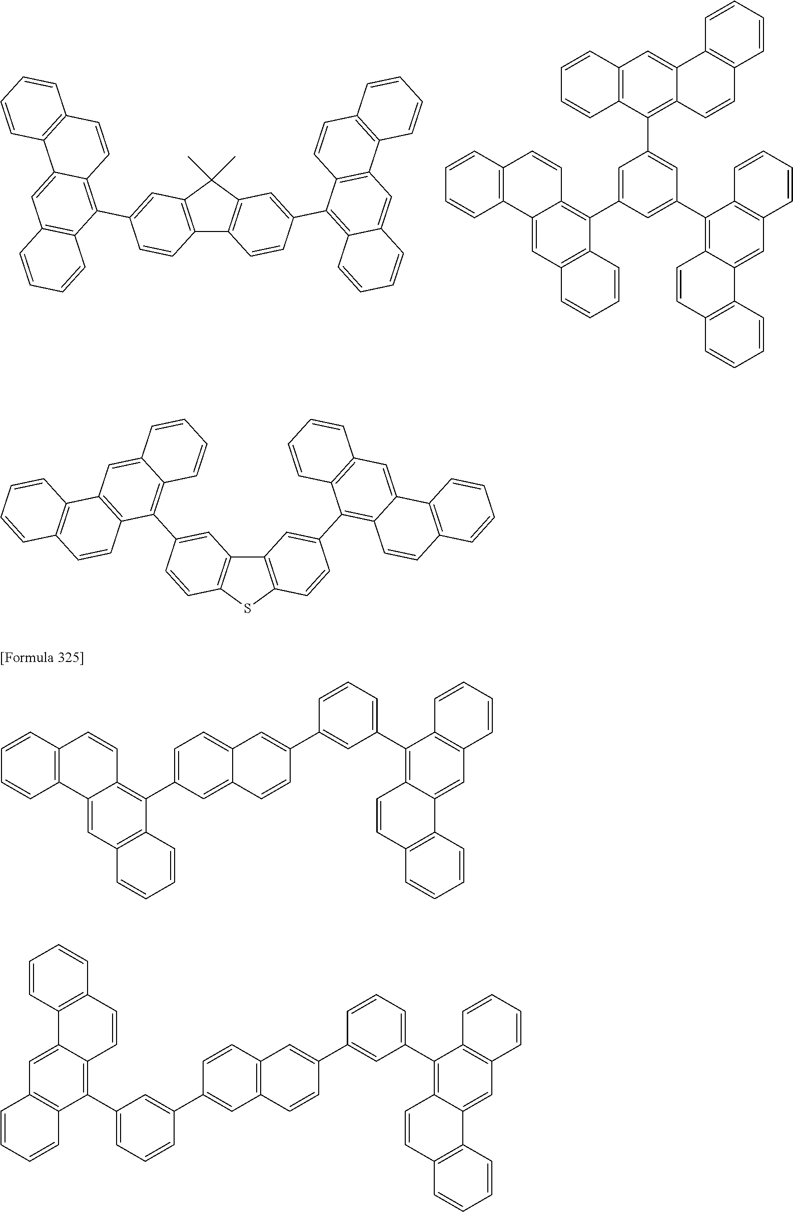

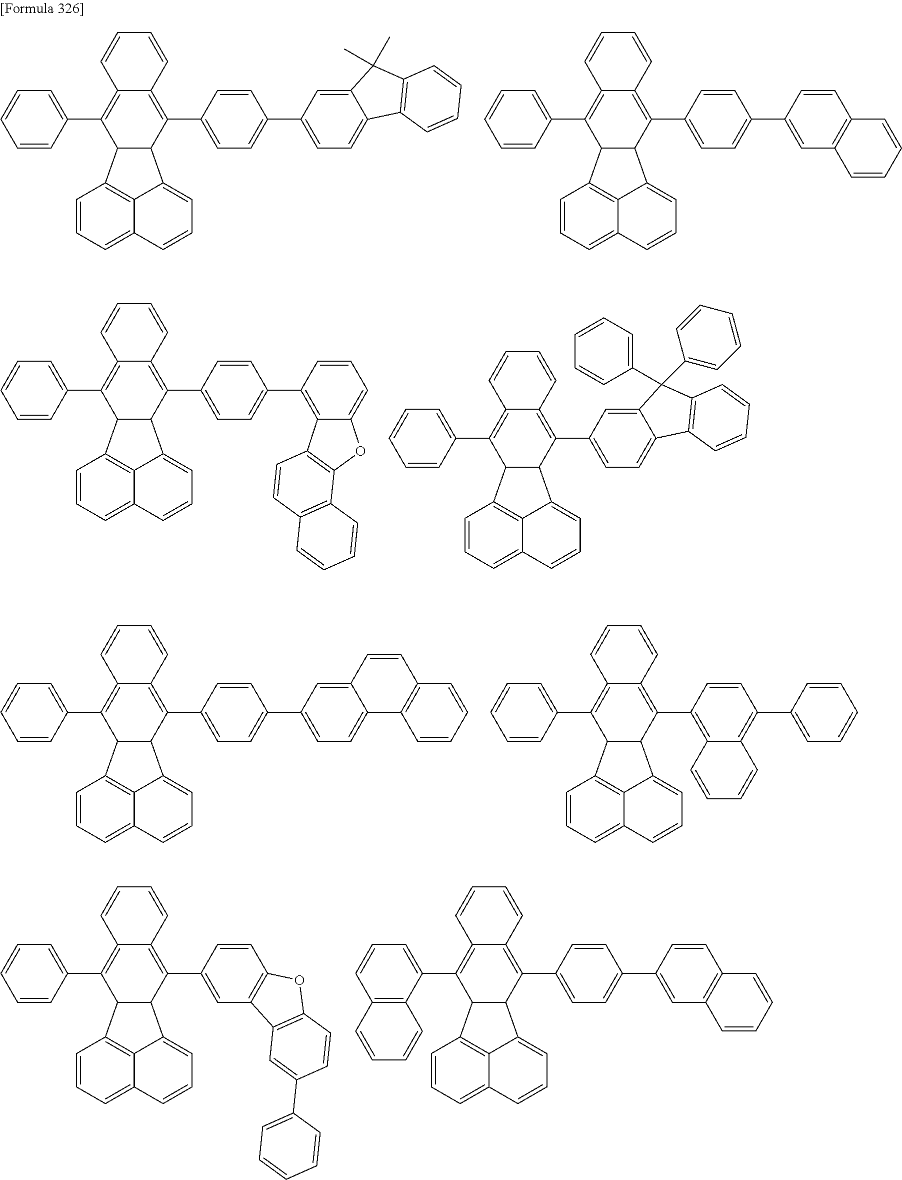

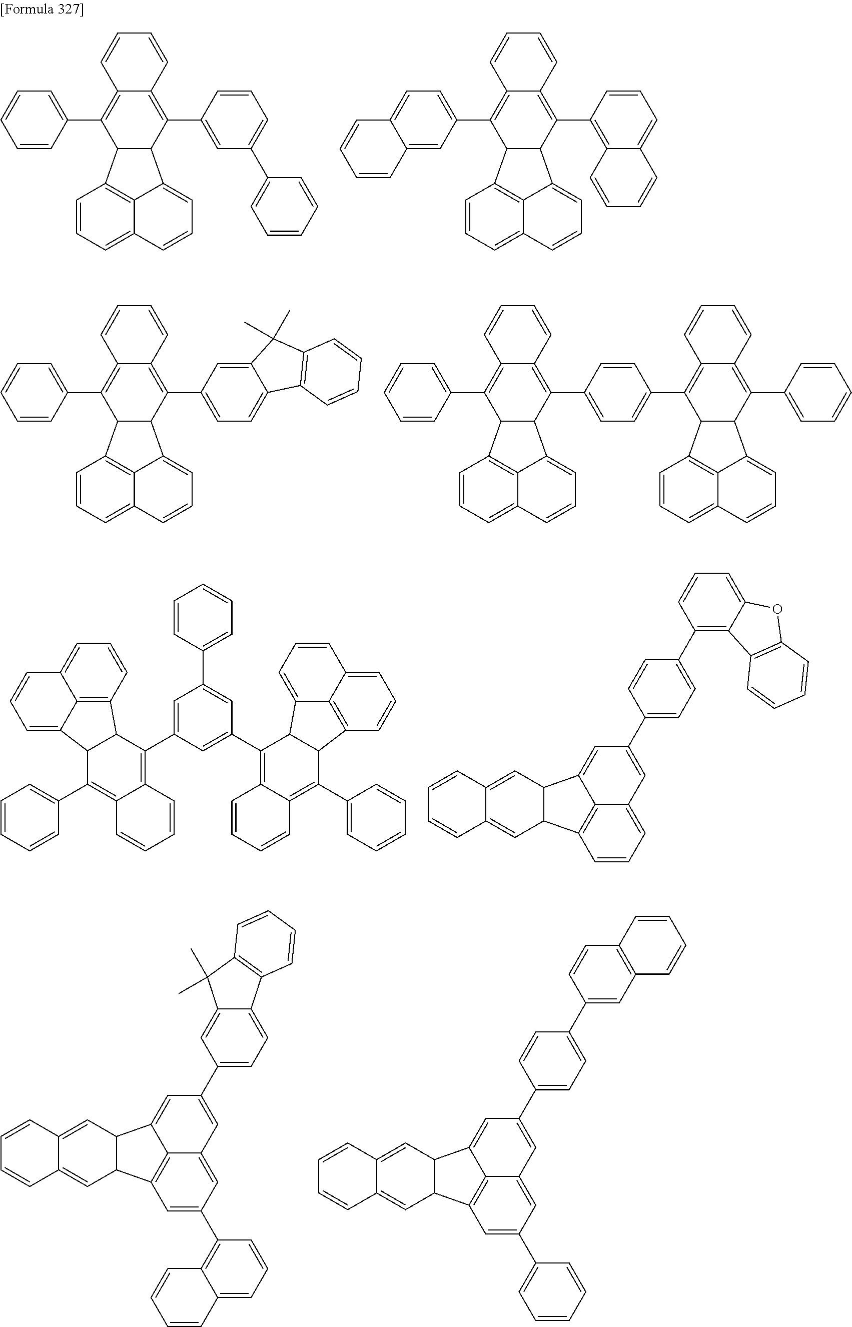

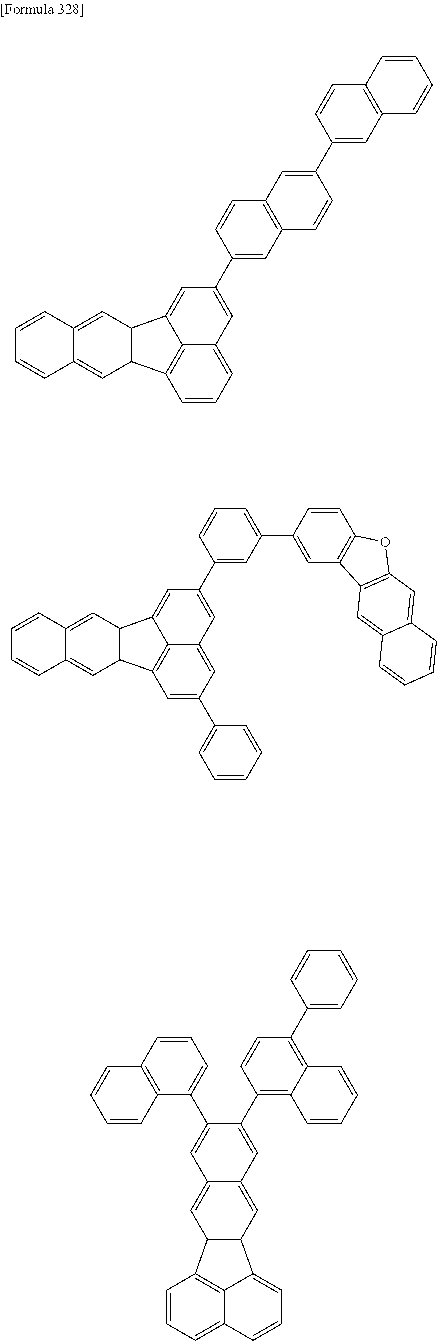

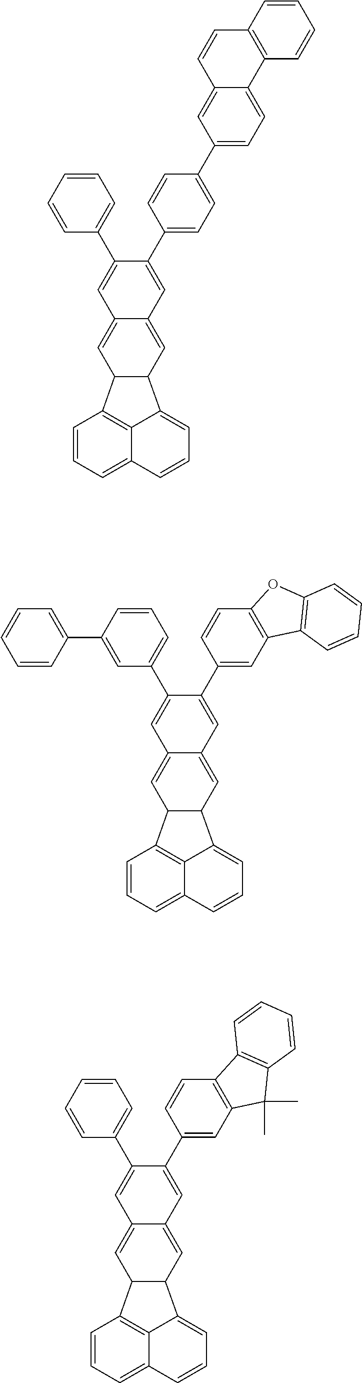

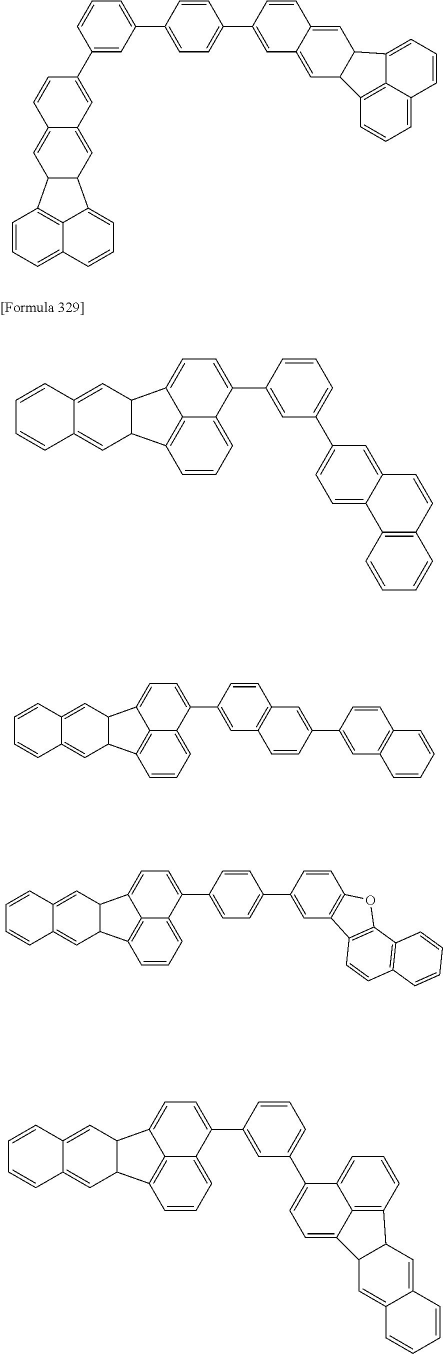

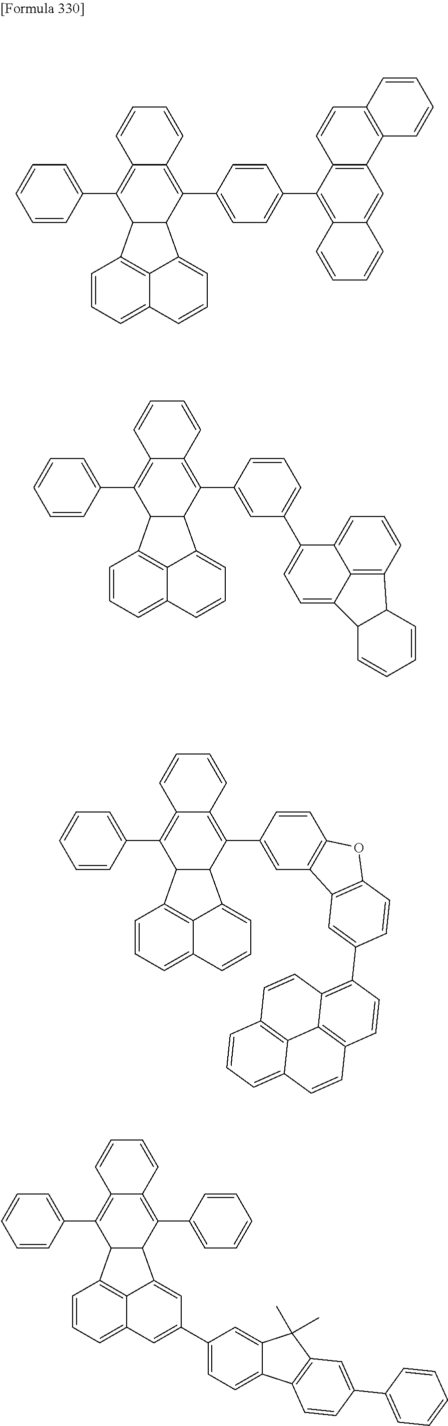

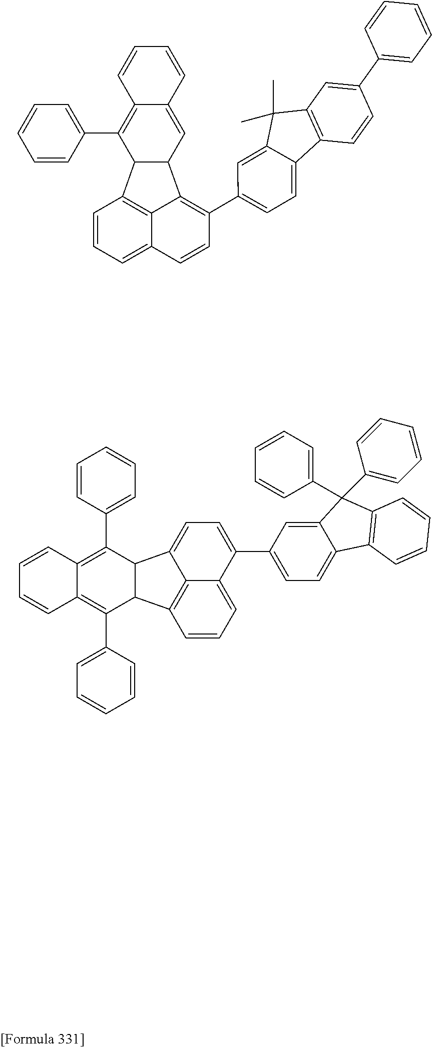

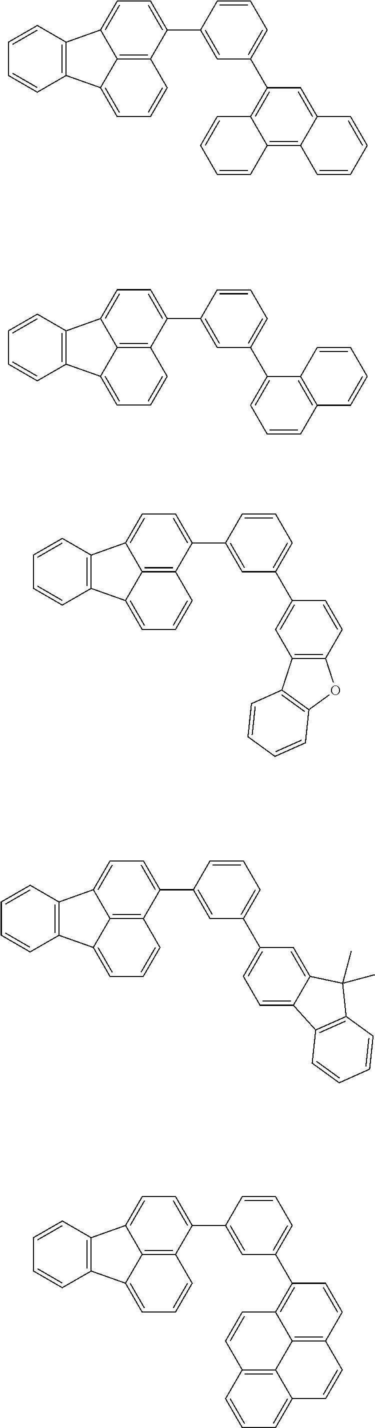

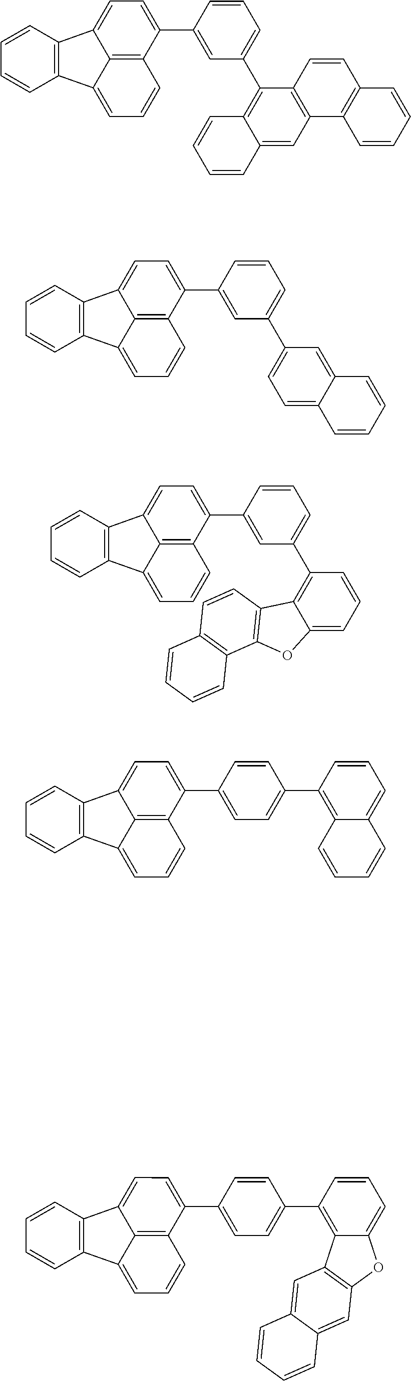

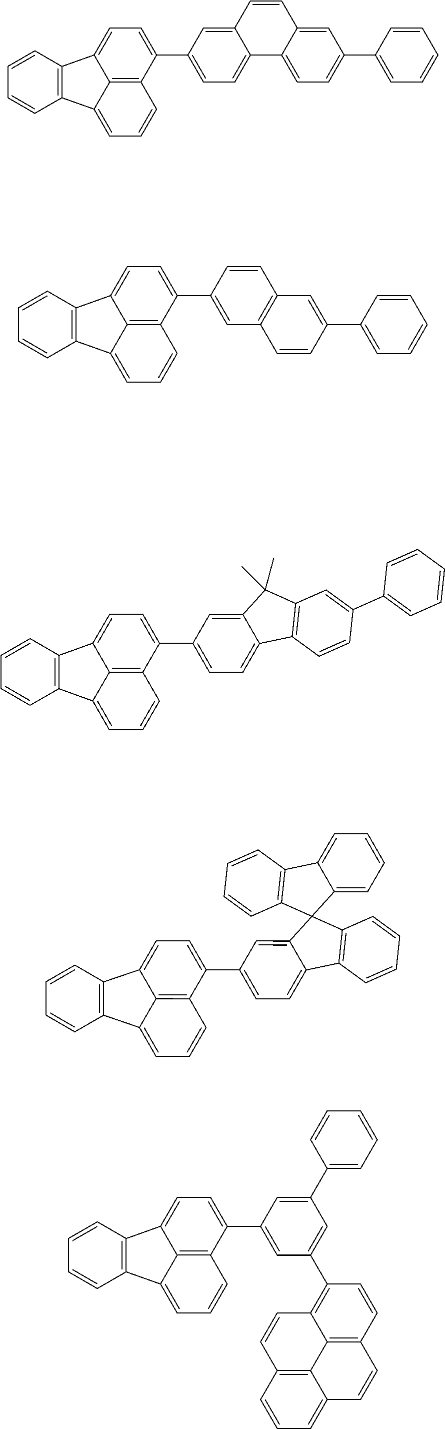

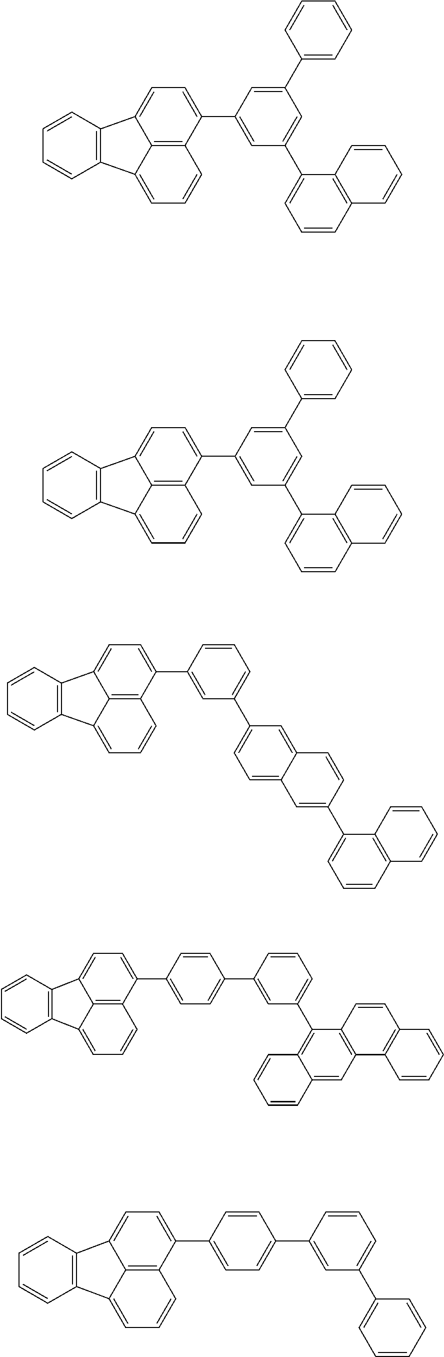

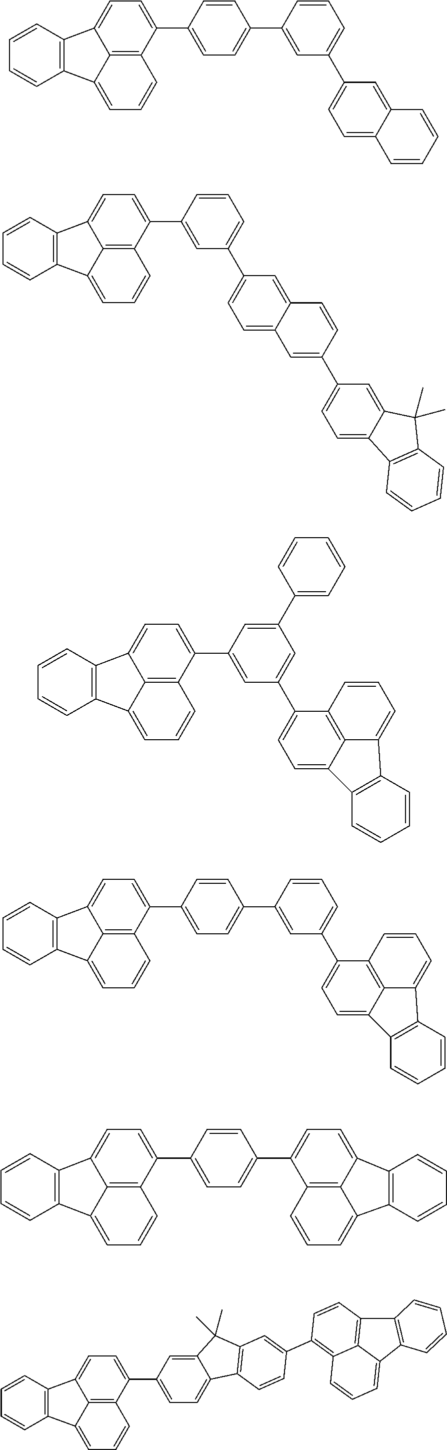

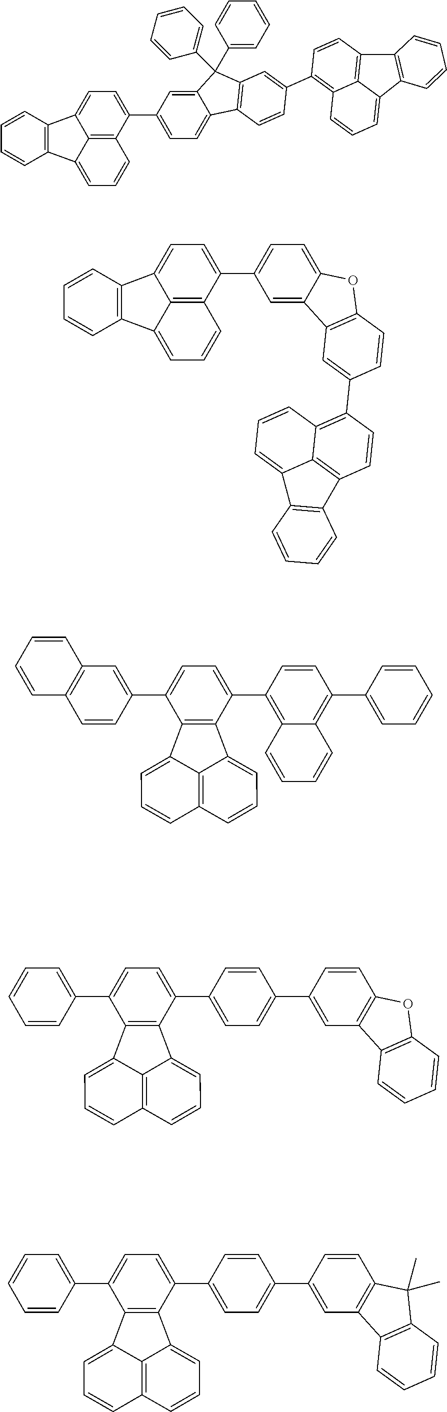

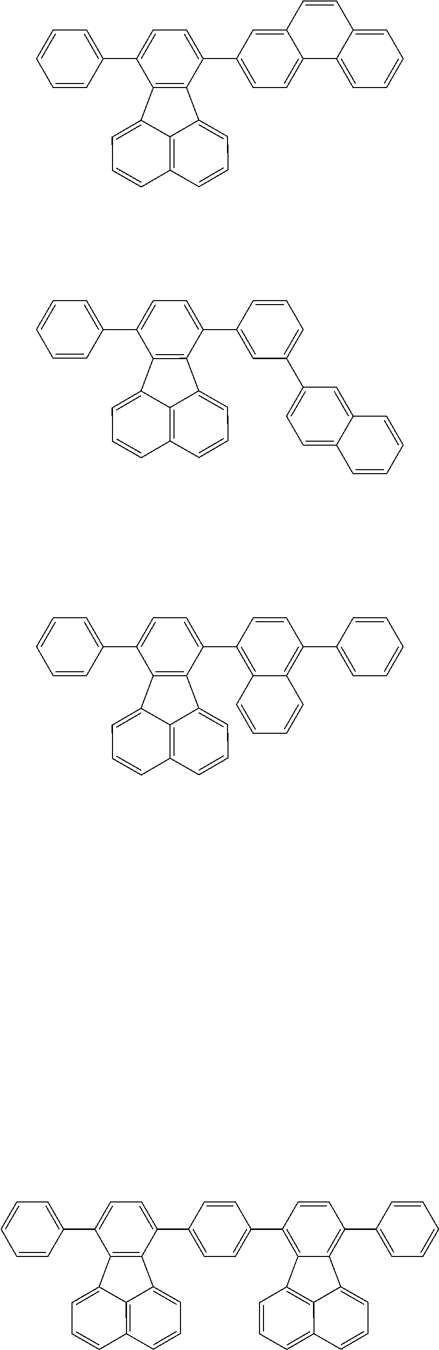

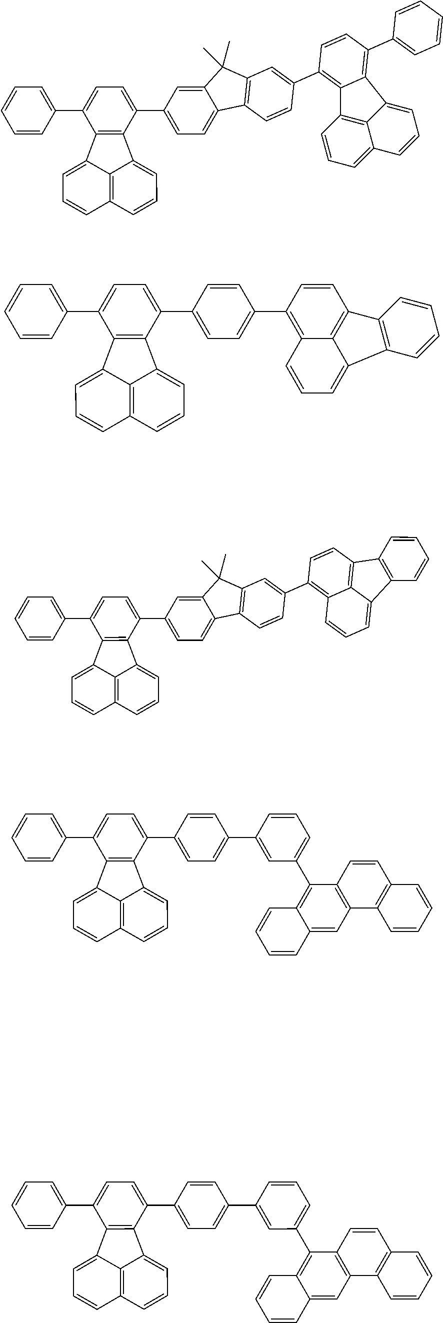

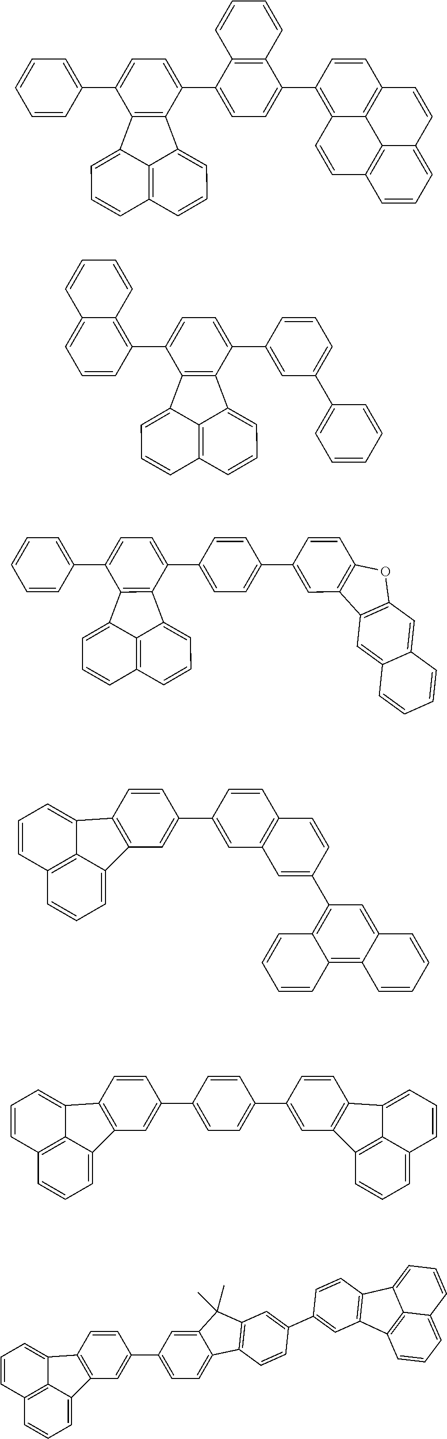

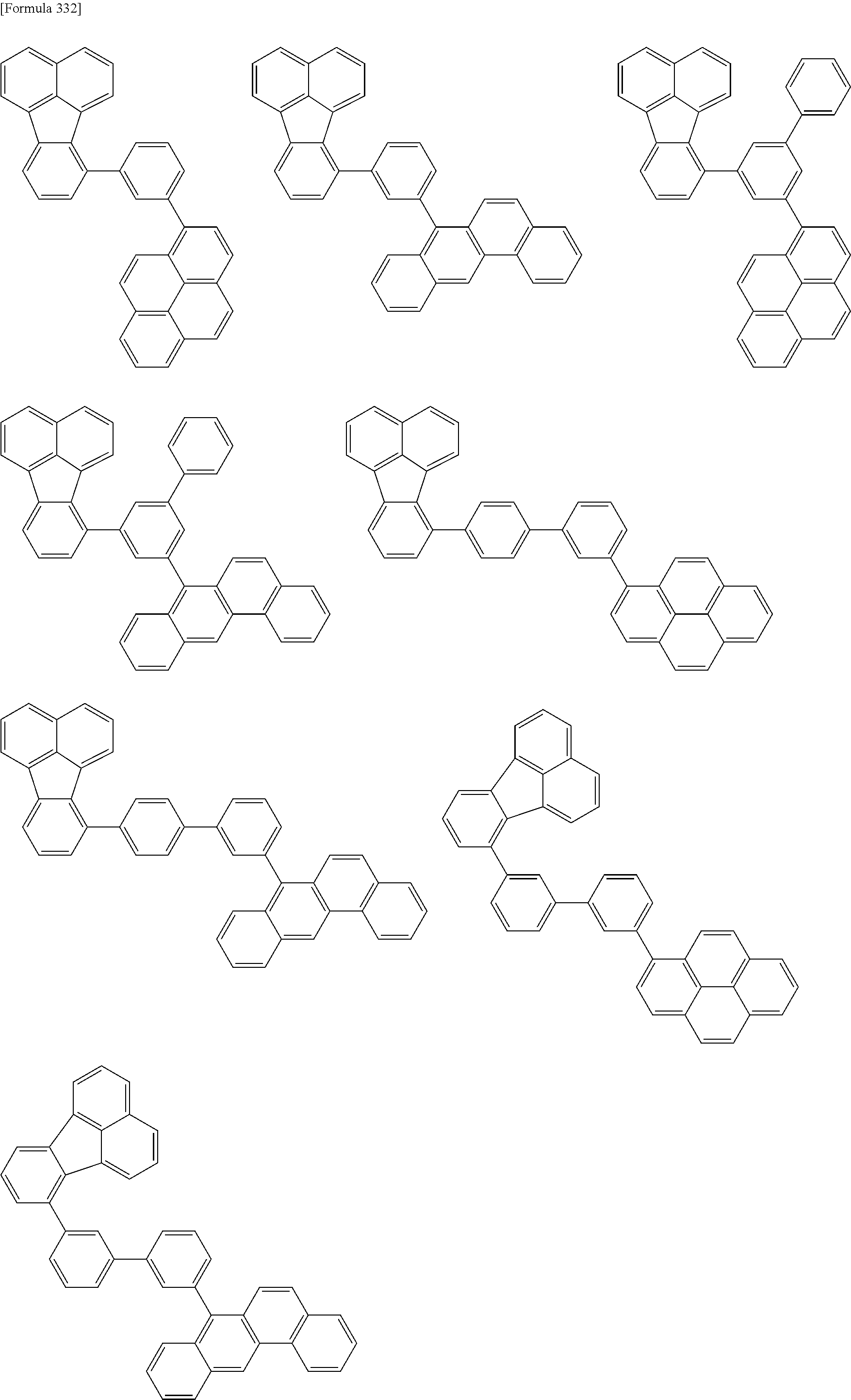

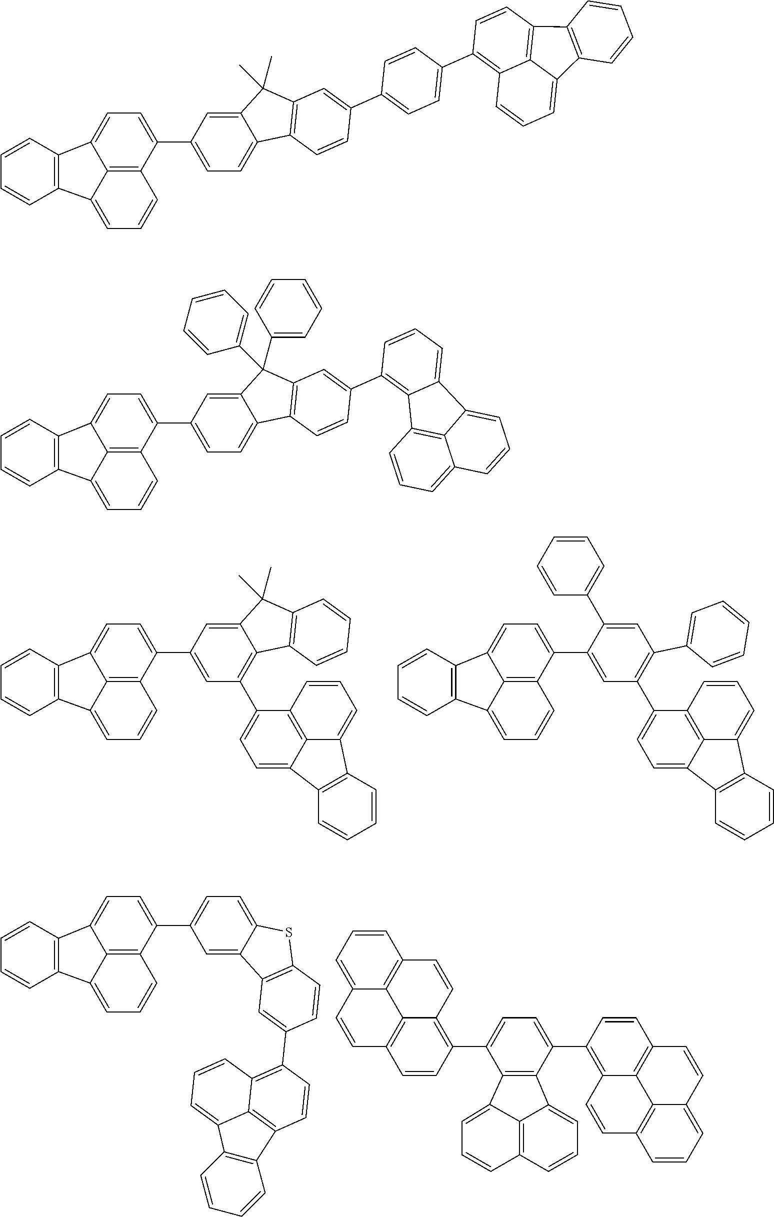

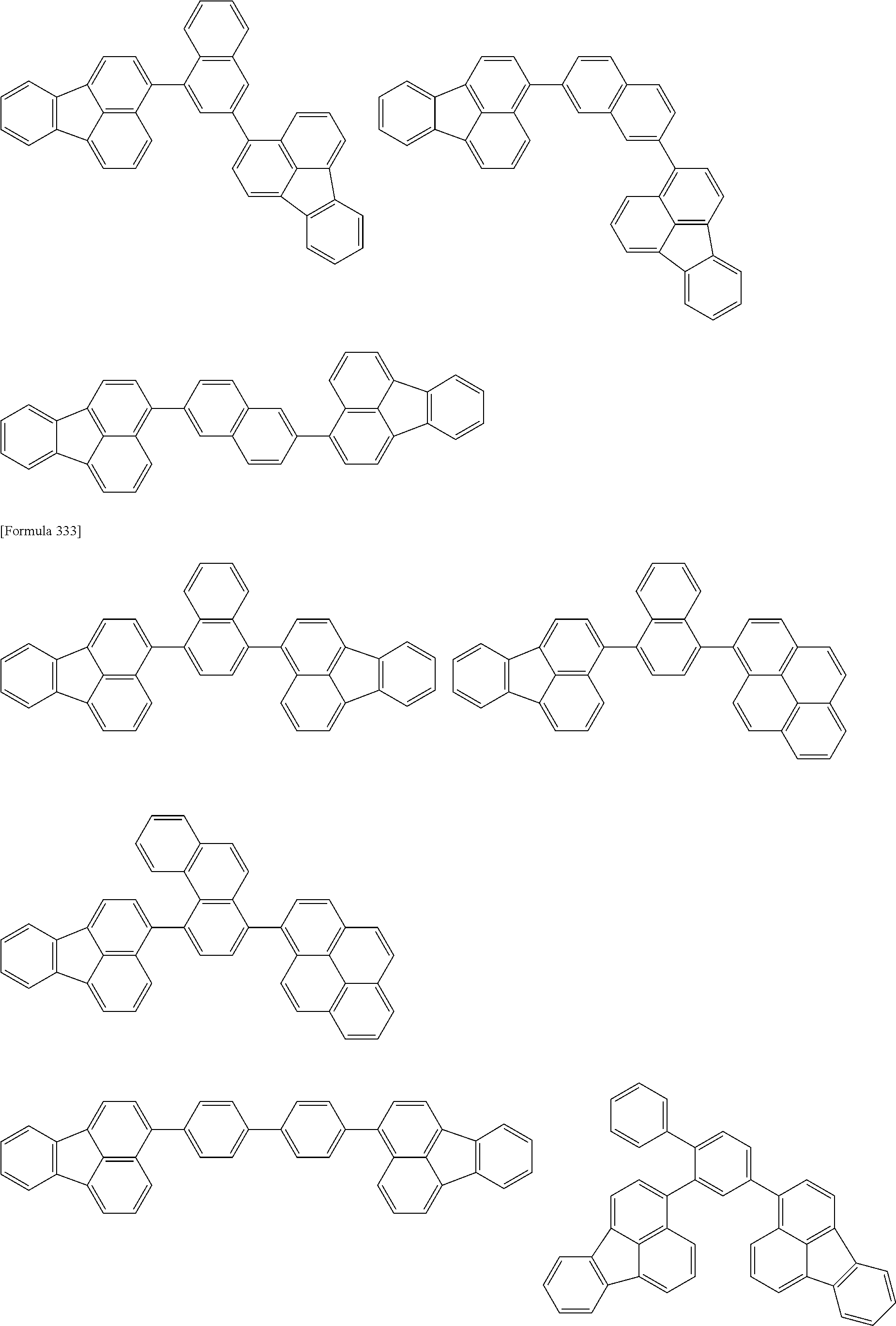

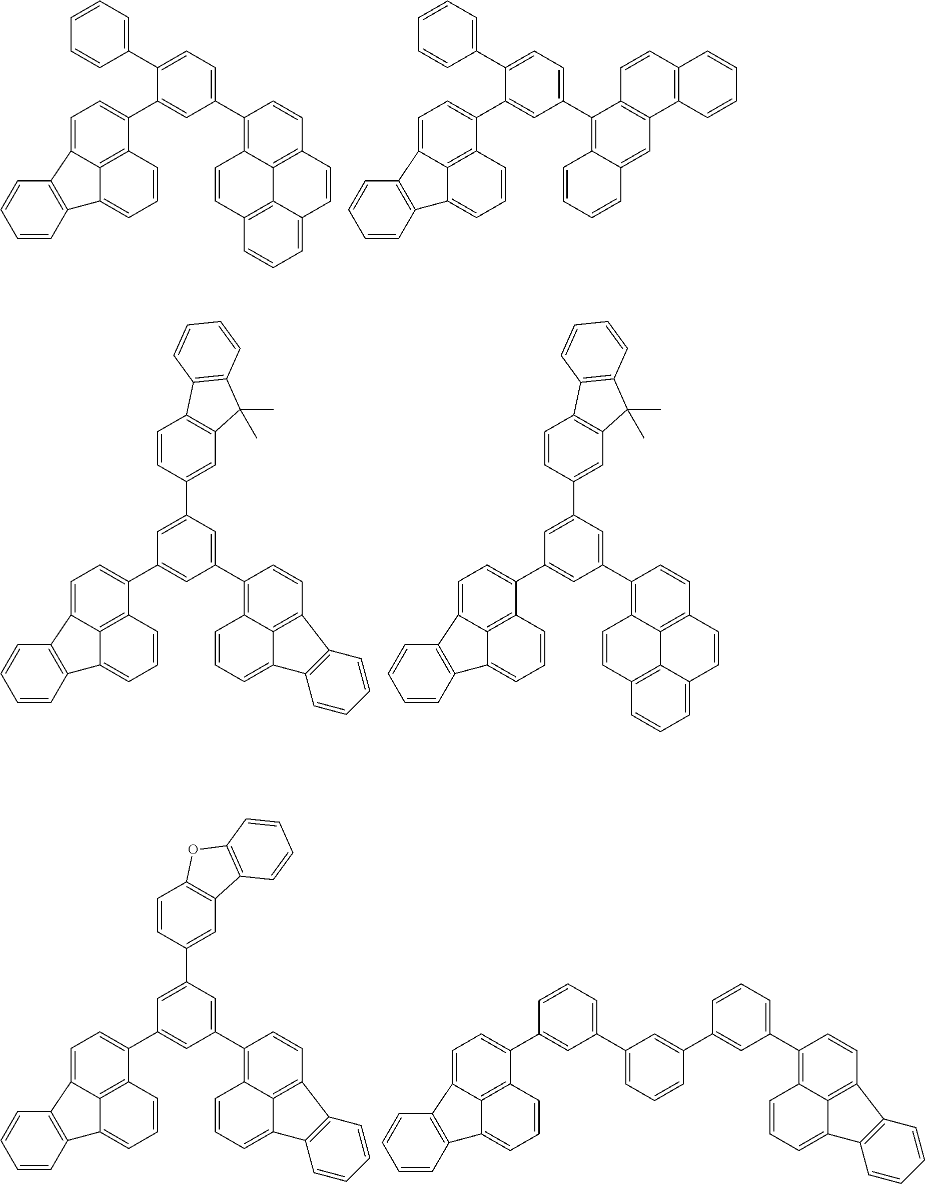

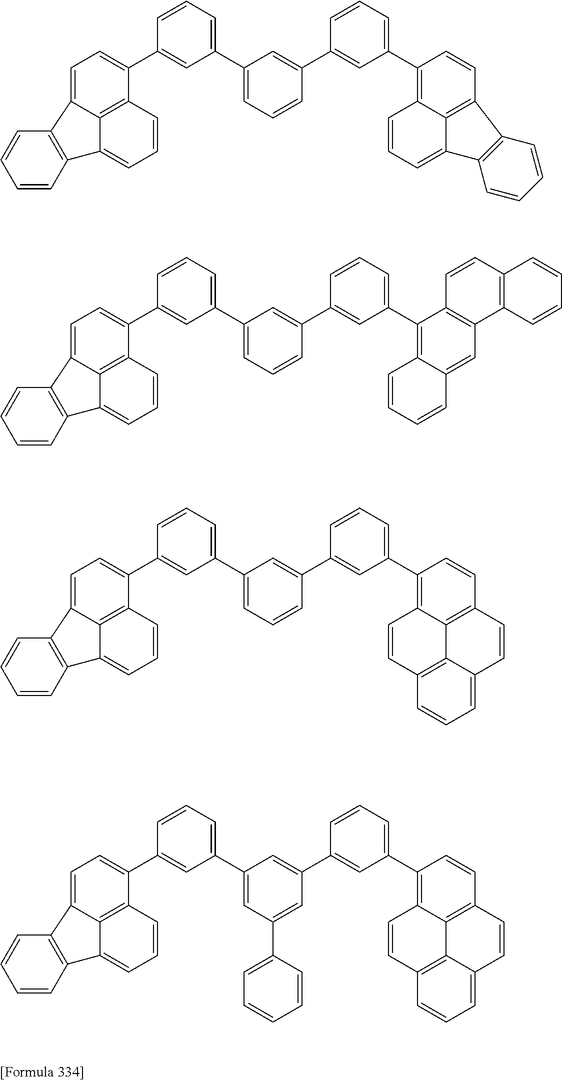

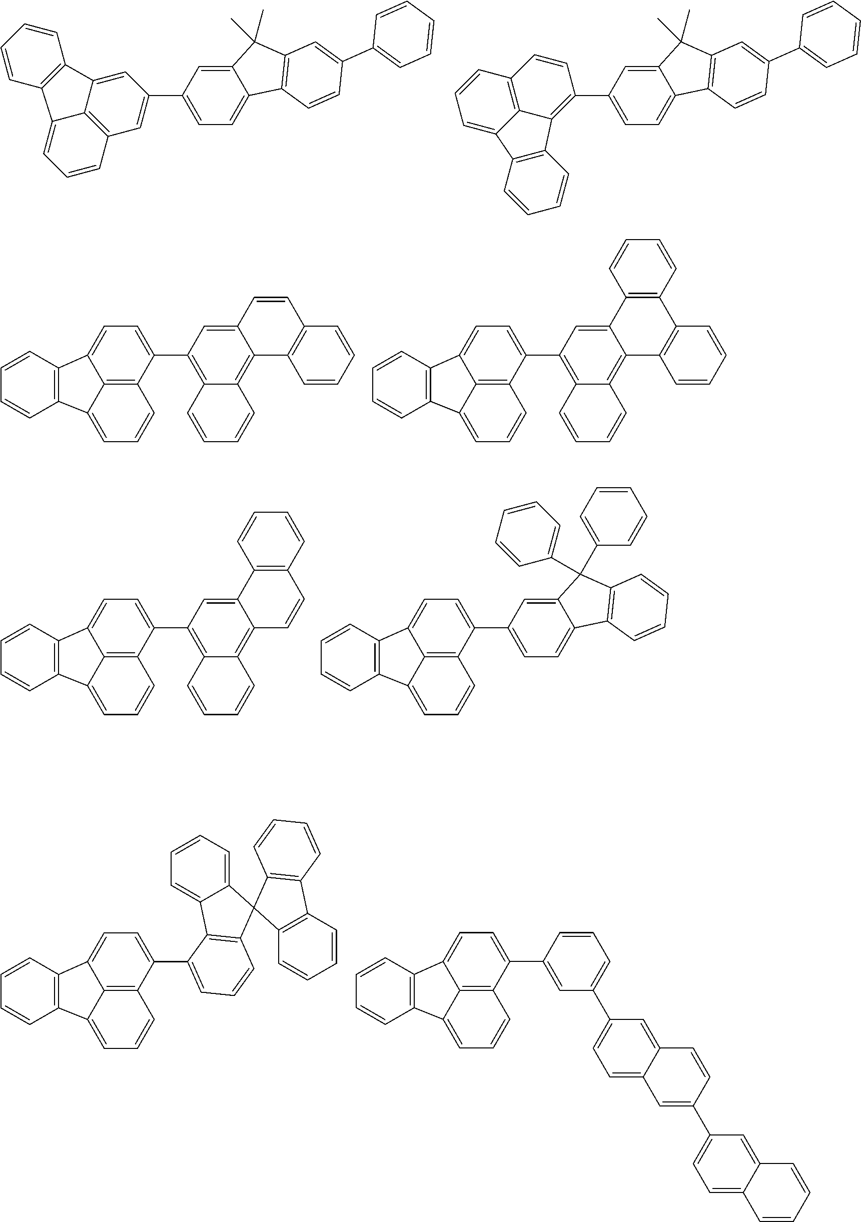

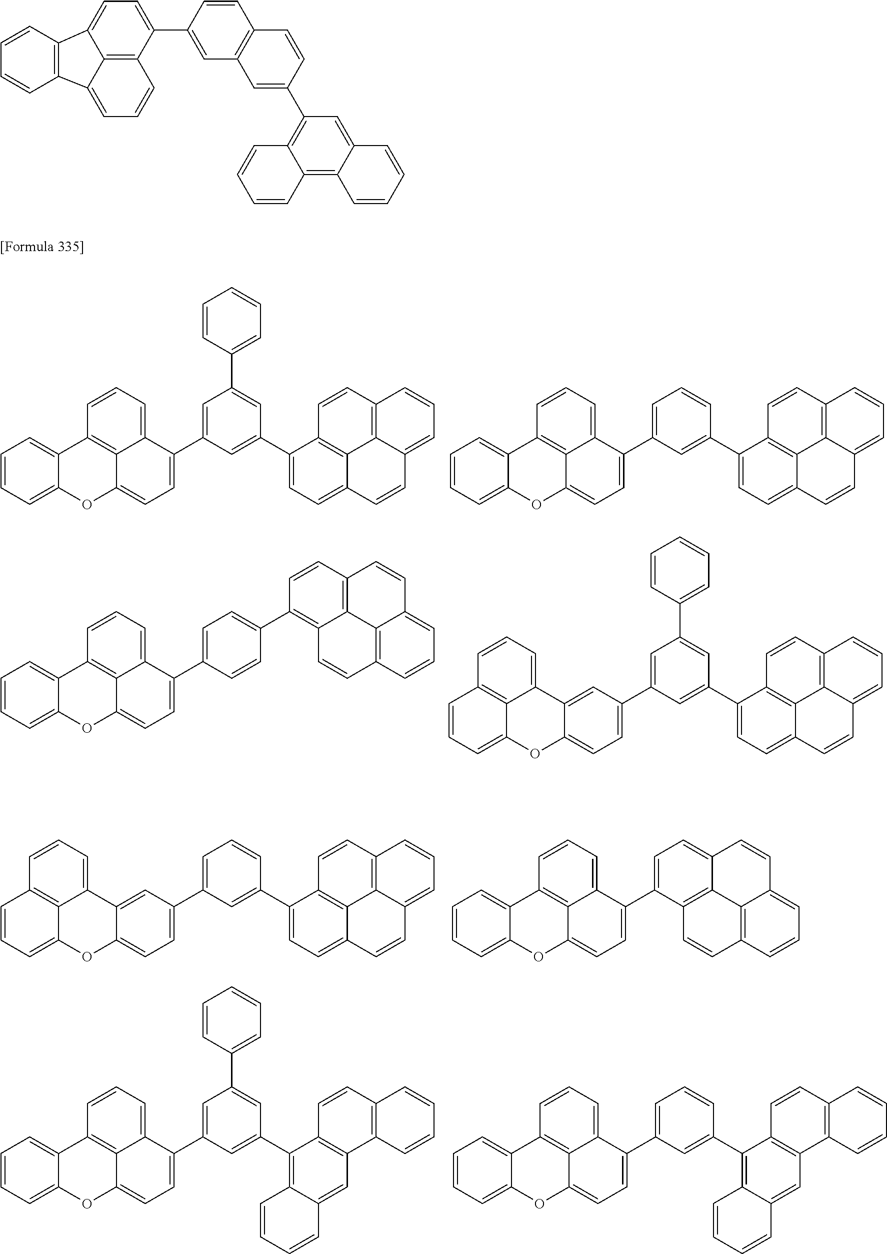

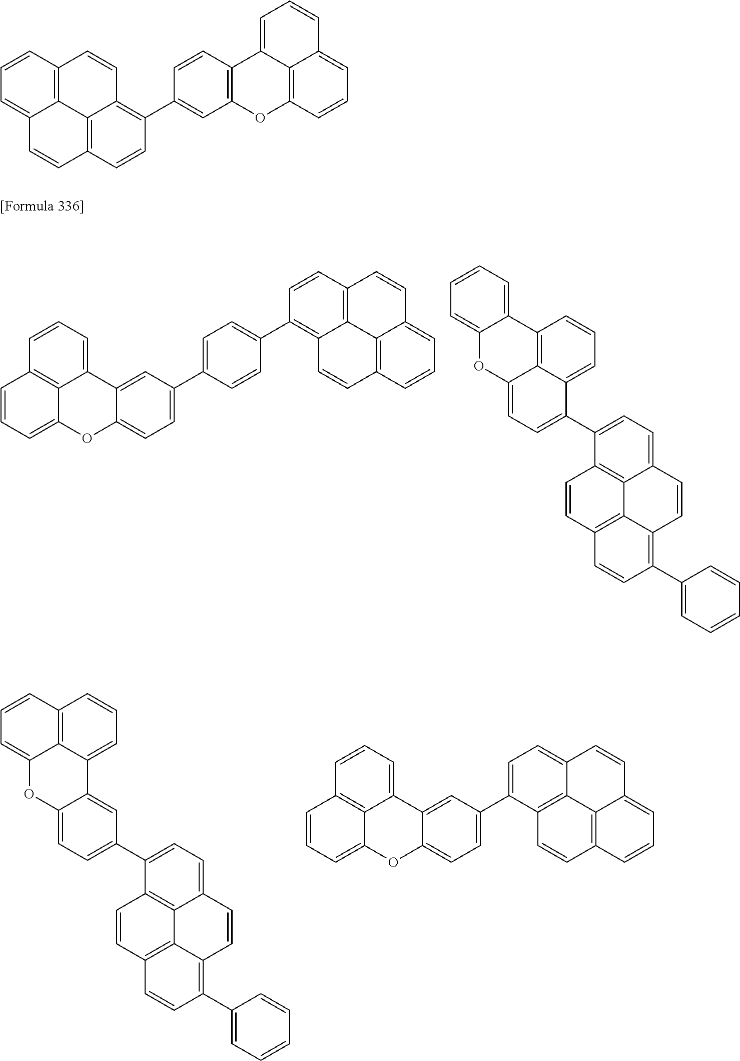

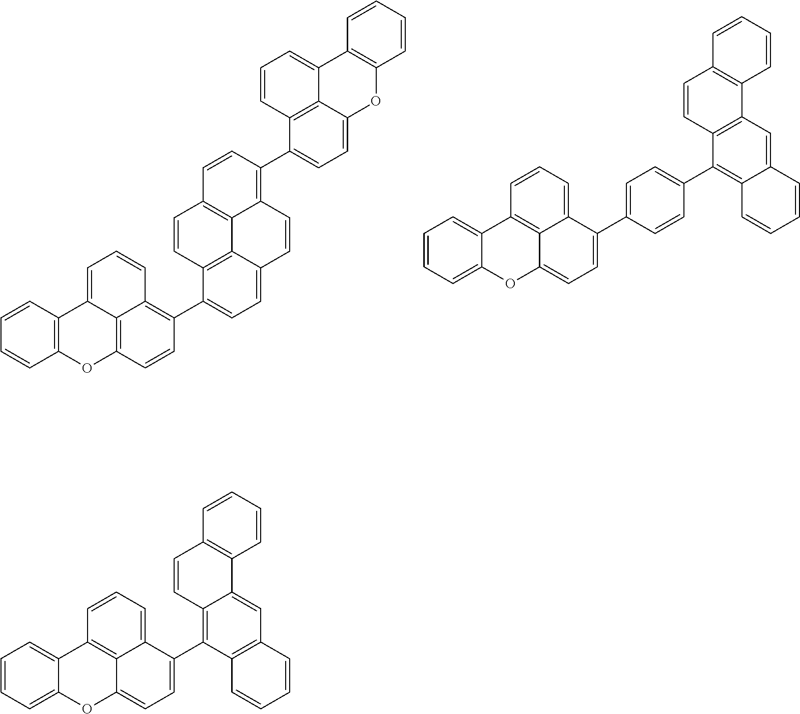 [SMILES: c1ccc2c(c1)Oc1ccc(-c3ccc4ccc5c(-c6ccc7c8c(cccc68)-c6ccccc6O7)ccc6ccc3c4c65)c3cccc-2c13 ; c1ccc2c(c1)Oc1ccc(-c3ccc(-c4c5ccccc5cc5c4ccc4ccccc45)cc3)c3cccc-2c13 ; c1ccc2c(c1)Oc1ccc(-c3c4ccccc4cc4c3ccc3ccccc34)c3cccc-2c13]

### First Luminescent Compound and Second Luminescent Compound

In an exemplary arrangement of the organic EL device according to the exemplary embodiment, examples of the first luminescent compound and the second luminescent compound, in addition to the exemplary compounds above (such as the compound represented by the formula (6)), include the third compound below and the fourth compound below.

The third compound and the fourth compound are each independently at least one compound selected from the group consisting of a compound represented by a formula (3), a compound represented by a formula (3X), a compound represented by a formula (4), a compound represented by a formula (5), a compound represented by a formula (7), a compound represented by a formula (8), a compound represented by a formula (9), and a compound represented by a formula (10) below.

### Compound Represented by Formula (3)

The compound represented by the formula (3) will be described below.

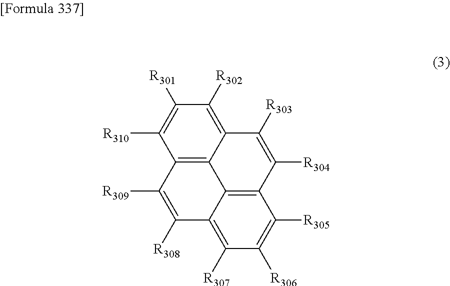

In the formula (3):
at least one combination of adjacent two or more of R₃₀₁ to R₃₁₀ are mutually bonded to form a substituted or unsubstituted monocyclic ring, mutually bonded to form a substituted or unsubstituted fused ring, or not mutually bonded; at least one of R₃₀₁ to R₃₁₀ is a monovalent group represented by a formula (31) below; and
R₃₀₁ to R₃₁₀ forming neither the monocyclic ring nor the fused ring and not being the monovalent group represented by the formula (31) are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

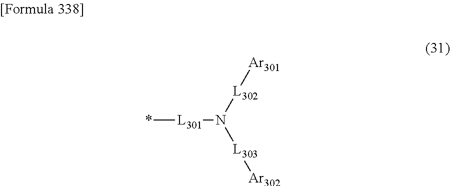

In the formula (31):
Ar₃₀₁ and Ar₃₀₂ are each independently a substituted or unsubstituted aryl group having 6 to 50 ring carbon atoms, or a substituted or unsubstituted heterocyclic group having 5 to 50 ring atoms;
L₃₀₁ to L₃₀₃ are each independently a single bond, a substituted or unsubstituted arylene group having 6 to 30 ring carbon atoms, or a substituted or unsubstituted divalent heterocyclic group having 5 to 30 ring atoms; and
* represents a bonding position to a pyrene ring in the formula (3).

In the third and fourth compounds, R₉₀₁, R₉₀₂, R₉₀₃, R₉₀₄, R₉₀₅, R₉₀₆, and R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms, preferably, a substituted or unsubstituted alkyl group having 1 to 50 carbon atoms, or a substituted or unsubstituted aryl group having 6 to 50 ring carbon atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different; and
when a plurality of R₉₀₇ are present, the plurality of R₉₀₇ are mutually the same or different.

In the formula (3), two of R₃₀₁ to R₃₁₀ are each preferably a group represented by the formula (31).

In an exemplary embodiment, the compound represented by the formula (3) is a compound represented by a formula (33) below.

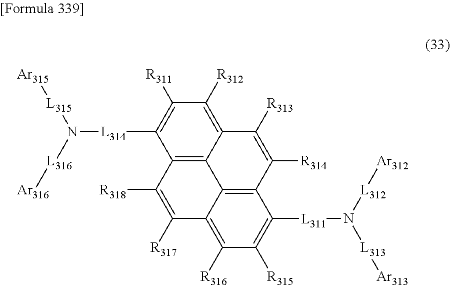

In the formula (33):
R₃₁₁ to R₃₁₈ each independently represent the same as R₃₀₁ to R₃₁₀ in the formula (3) not being the monovalent group represented by the formula (31);
L₃₁₁ to L₃₁₆ are each independently a single bond, a substituted or unsubstituted arylene group having 6 to 30 ring carbon atoms, or a substituted or unsubstituted divalent heterocyclic group having 5 to 30 ring atoms; and
Ar₃₁₂, Ar₃₁₃, Ar₃₁₅, and Ar₃₁₆ are each independently a substituted or unsubstituted aryl group having 6 to 50 ring carbon atoms, or a substituted or unsubstituted heterocyclic group having 5 to 50 ring atoms.

In the formula (31), L₃₀₁ is preferably a single bond, and L₃₀₂ and L₃₀₃ are each preferably a single bond.

In an exemplary embodiment, the compound represented by the formula (3) is represented by a formula (34) or a formula (35) below.

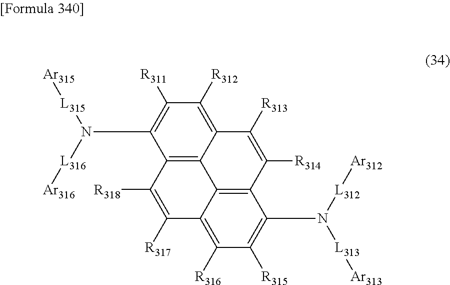

In the formula (34):
R₃₁₁ to R₃₁₈ each independently represent the same as R₃₀₁ to R₃₁₀ in the formula (3) not being the monovalent group represented by the formula (31);
L₃₁₂, L₃₁₃, L₃₁₅ and L₃₁₆ each independently represent the same as L₃₁₂, L₃₁₃, L₃₁₅ and L₃₁₆ in the formula (33); and
Ar₃₁₂, Ar₃₁₃, Ar₃₁₅ and Ar₃₁₆ each independently represent the same as Ar₃₁₂, Ar₃₁₃, Ar₃₁₅ and Ar₃₁₆ in the formula (33).

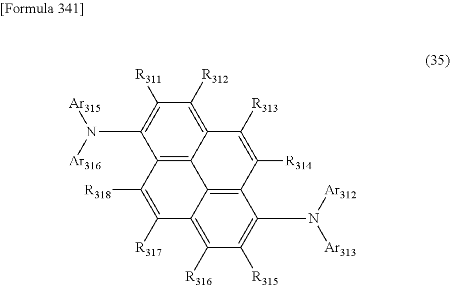

In the formula (35):
R₃₁₁ to R₃₁₈ each independently represent the same as R₃₀₁ to R₃₁₀ in the formula (3) not being the monovalent group represented by the formula (31); and
Ar₃₁₂, Ar₃₁₃, Ar₃₁₅ and Ar₃₁₆ each independently represent the same as Ar₃₁₂, Ar₃₁₃, Ar₃₁₅ and Ar₃₁₆ in the formula (33).

In the formula (31), at least one of Ar₃₀₁ or Ar₃₀₂ is preferably a group represented by a formula (36) below.

In the formulae (33) to (35), at least one of Ar₃₁₂ or Ar₃₁₃ is preferably a group represented by the formula (36).

In the formulae (33) to (35), at least one of Ar₃₁₅ or Ar₃₁₆ is preferably a group represented by the formula (36).

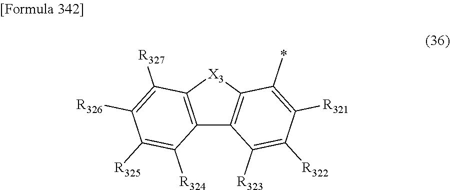

In the formula (36):
X₃ represents an oxygen atom or a sulfur atom;
at least one combination of adjacent two or more of R₃₂₁ to R₃₂₇ are mutually bonded to form a substituted or unsubstituted monocyclic ring, mutually bonded to form a substituted or unsubstituted fused ring, or not mutually bonded;
R₃₂₁ to R₃₂₇ forming neither the monocyclic ring nor the fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms; and
* represents a bonding position to L₃₀₂, L₃₀₃, L₃₁₂, L₃₁₃, L₃₁₅ or L₃₁₆.

X₃ is preferably an oxygen atom.

At least one of R₃₂₁ to R₃₂₇ is preferably a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

In the formula (31), preferably, Ar₃₀₁ is a group represented by the formula (36) and Ar₃₀₂ is a substituted or unsubstituted aryl group having 6 to 50 ring carbon atoms.

In the formulae (33) to (35), preferably, Ar₃₁₂ is a group represented by the formula (36) and Ar₃₁₃ is a substituted or unsubstituted aryl group having 6 to 50 ring carbon atoms.

In the formulae (33) to (35), preferably, Ar₃₁₅ is a group represented by the formula (36) and Ar₃₁₆ is a substituted or unsubstituted aryl group having 6 to 50 ring carbon atoms.

In an exemplary embodiment, the compound represented by the formula (3) is represented by a formula (37) below.

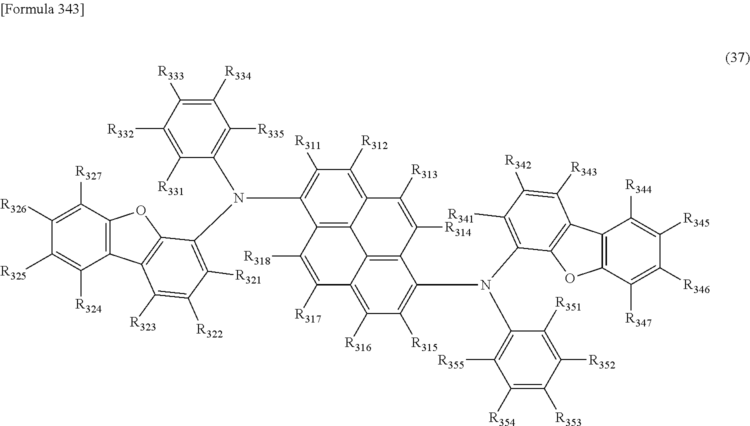

In the formula (37):
R₃₁₁ to R₃₁₈ each independently represent the same as R₃₀₁ to R₃₁₀ in the formula (3) not being the monovalent group represented by the formula (31);
at least one combination of adjacent two or more of R₃₂₁ to R₃₂₇ are mutually bonded to form a substituted or unsubstituted monocyclic ring, mutually bonded to form a substituted or unsubstituted fused ring, or not mutually bonded;
at least one combination of adjacent two or more of R₃₄₁ to R₃₄₇ are mutually bonded to form a substituted or unsubstituted monocyclic ring, mutually bonded to form a substituted or unsubstituted fused ring, or not mutually bonded;
R₃₂₁ to R₃₂₇ and R₃₄₁ to R₃₄₇ forming neither the monocyclic ring nor the fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms; and
R₃₃₁ to R₃₃₅ and R₃₅₁ to R₃₃₅ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

### Compound Represented by Formula (3X)

In an exemplary arrangement of the organic EL device according to the exemplary embodiment, the first compound is a compound represented by the formula (3X) below.

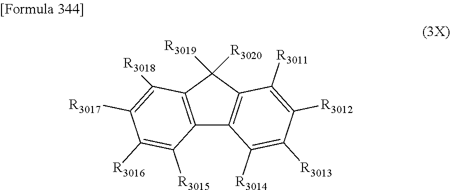

In the formula (3X):
at least one combination of adjacent two or more of R₃₀₁₁ to R₃₀₁₈ are mutually bonded to form a substituted or unsubstituted monocyclic ring, mutually bonded to form a substituted or unsubstituted fused ring, or not mutually bonded;
a combination of R₃₀₁₉ and R₃₀₂₀ are mutually bonded to form a substituted or unsubstituted monocyclic ring, mutually bonded to form a substituted or unsubstituted fused ring, or not mutually bonded;
at least one of R₃₀₁₁ to R₃₀₁₈ is a monovalent group represented by a formula (31X) below;
R₃₀₁₁ to R₃₀₁₈ forming neither the monocyclic ring nor the fused ring and not being the monovalent group represented by the formula (31X) are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms; and
R₃₀₁₉ and R₃₀₂₀ forming neither the monocyclic ring nor the fused ring are each independently a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

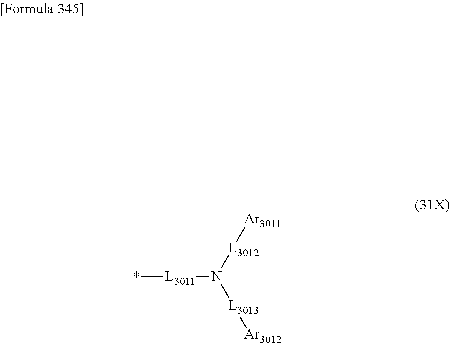

In the formula (31X):
Ar₃₀₁₁ and Ar₃₀₁₂ are each independently a substituted or unsubstituted aryl group having 6 to 50 ring carbon atoms, or a substituted or unsubstituted heterocyclic group having 5 to 50 ring atoms;
L₃₀₁₁ to L₃₀₁₃ are each independently a single bond, a substituted or unsubstituted arylene group having 6 to 30 ring carbon atoms, or a substituted or unsubstituted divalent heterocyclic group having 5 to 30 ring atoms; and
* represents a bonding position to a fluorene ring in the formula (3X).

In the third and fourth compounds, R₉₀₁, R₉₀₂, R₉₀₃, R₉₀₄, R₉₀₅, R₉₀₆, and R₉₀₇ each independently represent the same as R₉₀₁, R₉₀₂, R₉₀₃, R₉₀₄, R₉₀₅, R₉₀₆, and R₉₀₇ in the formula (3).

In the formula (3X), two of R₃₀₁₁ to R₃₀₁₈ are each preferably a group represented by the formula (31X).

In the formula (3X), it is preferable that a combination of R₃₀₁₃ and R₃₀₁₄ are mutually bonded to form a substituted or unsubstituted monocyclic ring or mutually bonded to form a substituted or unsubstituted fused ring.

In the formula (3X), it is preferable that a combination of R₃₀₁₅ and R₃₀₁₆ are mutually bonded to form a substituted or unsubstituted monocyclic ring or mutually bonded to form a substituted or unsubstituted fused ring.

In the formula (3X), R₃₀₁₉ and R₃₀₂₀ are each independently preferably a substituted or unsubstituted aryl group having 6 to 50 ring carbon atoms, or a substituted or unsubstituted heterocyclic group having 5 to 50 ring atoms, more preferably a substituted or unsubstituted aryl group having 6 to 50 ring carbon atoms.

In an exemplary embodiment, the compound represented by the formula (3X) is a compound represented by a formula (33X) below.

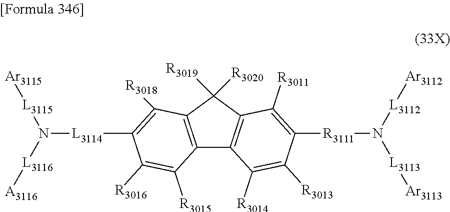

In the formula (33X):
R₃₀₁₁, R₃₀₁₃ to R₃₀₁₆ and R₃₀₁₈ each independently represent the same as R₃₀₁₁ to R₃₀₁₈ in the formula (3X) not being the monovalent group represented by the formula (31X);
R₃₀₁₉ and R₃₀₂₀ each independently represent the same as R₃₀₁₉ and R₃₀₂₀ forming neither the monocyclic ring nor the fused ring in the formula (3X);
L₃₁₁₁ to L₃₁₁₆ are each independently a single bond, a substituted or unsubstituted arylene group having 6 to 30 ring carbon atoms, or a substituted or unsubstituted divalent heterocyclic group having 5 to 30 ring atoms; and
Ar₃₁₁₂, Ar₃₁₁₃, Ar₃₁₁₅, and Ar₃₁₁₆ are each independently a substituted or unsubstituted aryl group having 6 to 50 ring carbon atoms, or a substituted or unsubstituted heterocyclic group having 5 to 50 ring atoms.

In the formula (31X), L₃₀₁₁ is preferably a single bond, and L₃₀₁₂ and L₃₀₁₃ are each preferably a single bond.

In an exemplary embodiment, the compound represented by the formula (3X) is represented by a formula (34X) or a formula (35X) below.

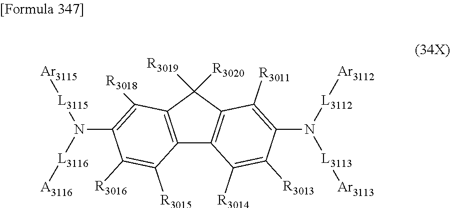

In the formula (34X):
R₃₀₁₁, R₃₀₁₃ to R₃₀₁₆ and R₃₀₁₈ each independently represent the same as R₃₀₁₁ to R₃₀₁₈ in the formula (3X) not being the monovalent group represented by the formula (31X);
R₃₀₁₉ and R₃₀₂₀ each independently represent the same as R₃₀₁₉ and R₃₀₂₀ forming neither the monocyclic ring nor the fused ring in the formula (3X);
L₃₁₁₂, L₃₁₁₃, L₃₁₁₅ and L₃₁₁₆ each independently represent the same as L₃₁₁₂, L₃₁₁₃, L₃₁₁₅ and L₃₁₁₆ in the formula (33X); and
Ar₃₁₁₂, Ar₃₁₁₃, Ar₃₁₁₅ and Ar₃₁₁₆ each independently represent the same as Ar₃₁₁₂, Ar₃₁₁₃, Ar₃₁₁₅ and Ar₃₁₁₆ in the formula (33X).

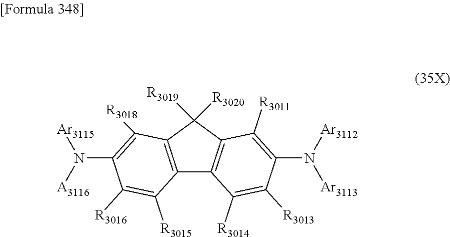

In the formula (35X):
R₃₀₁₁, R₃₀₁₃ to R₃₀₁₆ and R₃₀₁₈ each independently represent the same as R₃₀₁₁ to R₃₀₁₈ in the formula (3X) not being the monovalent group represented by the formula (31X);
R₃₀₁₉ and R₃₀₂₀ each independently represent the same as R₃₀₁₉ and R₃₀₂₀ forming neither the monocyclic ring nor the fused ring in the formula (3X); and
Ar₃₁₁₂, Ar₃₁₁₃, Ar₃₁₁₅ and Ar₃₁₁₆ each independently represent the same as Ar₃₁₁₂, Ar₃₁₁₃, Ar₃₁₁₅ and Ar₃₁₁₆ in the formula (33X).

In the formula (31X), at least one of Ar₃₀₁₁ or Ar₃₀₁₂ is preferably a group represented by a formula (36X) below.

In the formulae (33X) to (35X), at least one of Ar₃₁₁₂ or Ar₃₁₁₃ is preferably a group represented by the formula (36X).

In the formulae (33X) to (35X), at least one of Ar₃₁₁₅ or Ar₃₁₁₆ is preferably a group represented by the formula (36X).

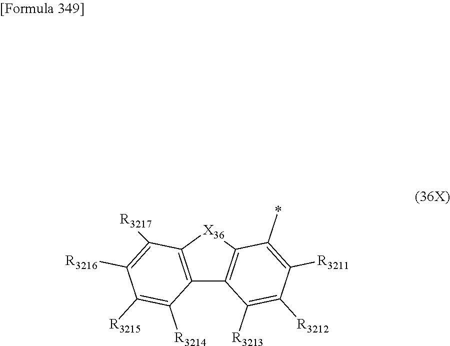

In the formula (36X):
X₃₆ represents an oxygen atom or a sulfur atom;
at least one combination of adjacent two or more of R₃₂₁₁ to R₃₂₁₇ are mutually bonded to form a substituted or unsubstituted monocyclic ring, mutually bonded to form a substituted or unsubstituted fused ring, or not mutually bonded;
R₃₂₁₁ to R₃₂₁₇ forming neither the monocyclic ring nor the fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms; and
* represents a bonding position to L₃₀₁₂, L₃₀₁₃, L₃₁₁₂, L₃₁₁₃, L₃₁₁₅ or L₃₁₁₆.

X₃₆ is preferably an oxygen atom.

At least one of R₃₂₁₁ to R₃₂₁₇ is preferably a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

In the formula (31X), preferably, Ar₃₀₁₁ is a group represented by the formula (36X) and Ar₃₀₁₂ is a substituted or unsubstituted aryl group having 6 to 50 ring carbon atoms.

In the formulae (33X) to (35X), preferably, Ar₃₁₁₂ is a group represented by the formula (36X) and Ar₃₁₁₃ is a substituted or unsubstituted aryl group having 6 to 50 ring carbon atoms.

In the formulae (33X) to (35X), preferably, Ar₃₁₁₅ is a group represented by the formula (36X) and Ar₃₁₁₆ is a substituted or unsubstituted aryl group having 6 to 50 ring carbon atoms.

In an exemplary embodiment, the compound represented by the formula (3X) is represented by a formula (37X) below.

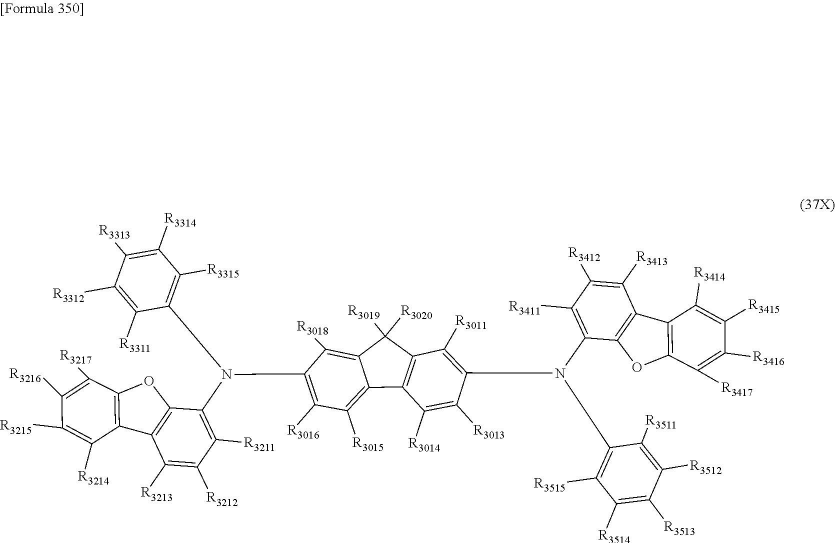

In the formula (37X):
R₃₀₁₁, R₃₀₁₃ to R₃₀₁₆ and R₃₀₁₈ each independently represent the same as R₃₀₁₁ to R₃₀₁₈ in the formula (3X) not being the monovalent group represented by the formula (31X);
R₃₀₁₉ and R₃₀₂₀ each independently represent the same as R₃₀₁₉ and R₃₀₂₀ forming neither the monocyclic ring nor the fused ring in the formula (3X);
at least one combination of adjacent two or more of R₃₂₁₁ to R₃₂₁₇ are mutually bonded to form a substituted or unsubstituted monocyclic ring, mutually bonded to form a substituted or unsubstituted fused ring, or not mutually bonded;
at least one combination of adjacent two or more of R₃₄₁₁ to R₃₄₁₇ are mutually bonded to form a substituted or unsubstituted monocyclic ring, mutually bonded to form a substituted or unsubstituted fused ring, or not mutually bonded;
R₃₂₁₁ to R₃₂₁₇ and R₃₄₁₁ to R₃₄₁₇ forming neither the monocyclic ring nor the fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms; and
R₃₃₁₁ to R₃₃₁₅ and R₃₅₁₁ to R₃₅₁₅ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In an exemplary embodiment, the compound represented by the formula (3X) is represented by a formula (38X) or (39X) below.

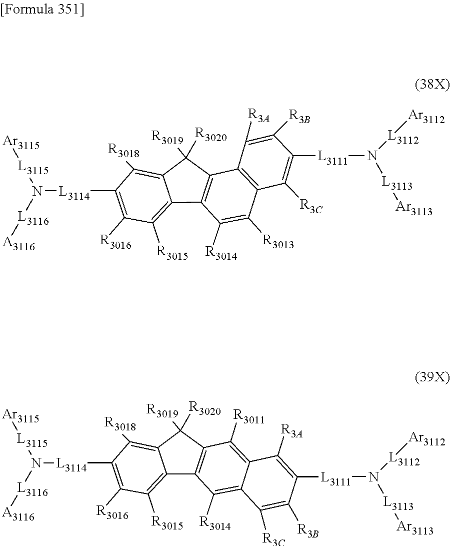

In the formulae (38X) and (39X):
R₃₀₁₁, R₃₀₁₃ to R₃₀₁₆, R₃₀₁₈, R_{3A}, R_{3B} and R_{3C} each independently represent the same as R₃₀₁₁ in the formula (3X) not being the monovalent group represented by the formula (31X);
R₃₀₁₉ and R₃₀₂₀ each independently represent the same as R₃₀₁₉ and R₃₀₂₀ forming neither the monocyclic ring nor the fused ring in the formula (3X); and
L₃₁₁₁ to L₃₁₁₆, Ar₃₁₁₂, Ar₃₁₁₃, Ar₃₁₁₅ and Ar₃₁₁₆ each independently represent the same as L₃₁₁₁ to L₃₁₁₆, Ar₃₁₁₂, Ar₃₁₁₃, Ar₃₁₁₅ and Ar₃₁₁₆ in the formula (33X).

Specific Examples of Compounds Represented by Formulae (3) and (3X) Specific examples of the compounds represented by the formulae (3) and (3X) include compounds as below.

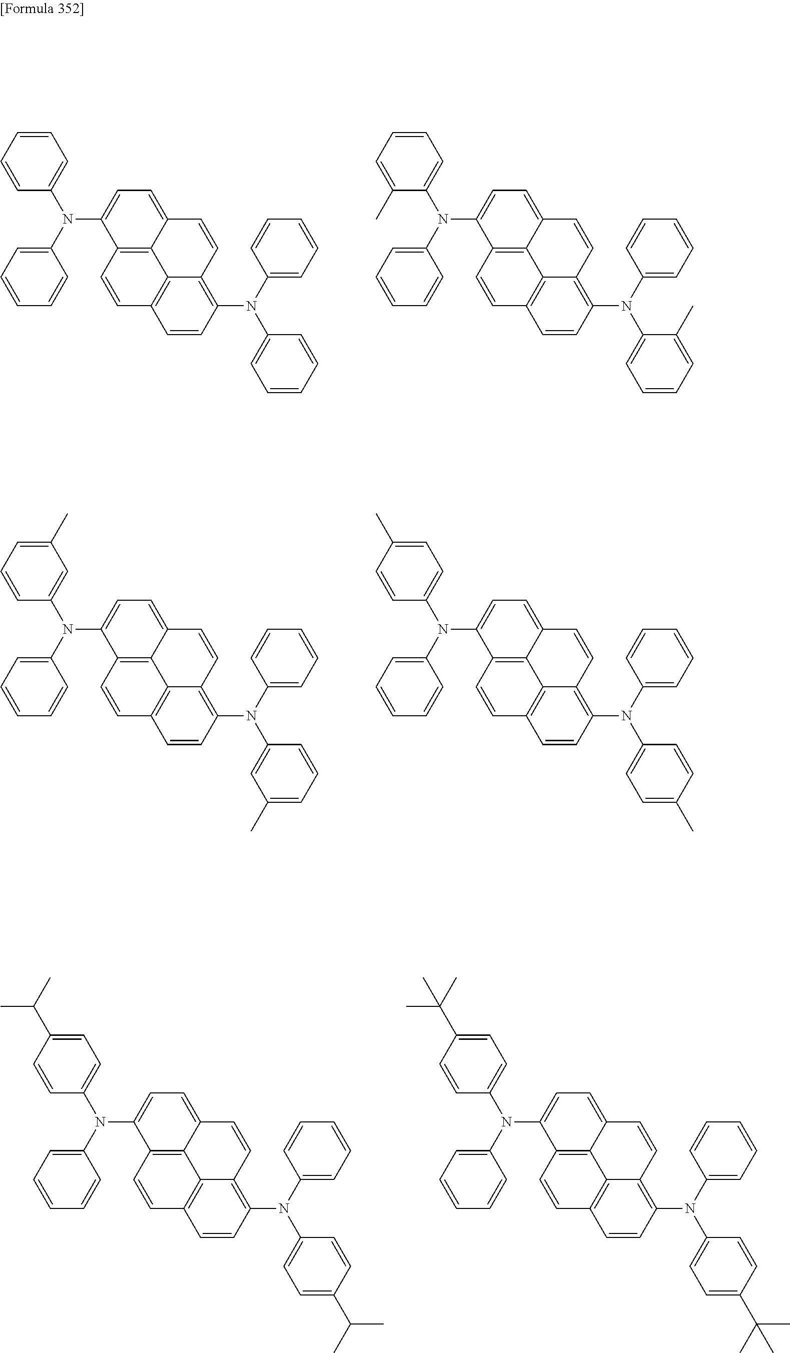 [SMILES: c1ccc(N(c2ccccc2)c2ccc3ccc4c(N(c5ccccc5)c5ccccc5)ccc5ccc2c3c54)cc1 ; Cc1ccccc1N(c1ccccc1)c1ccc2ccc3c(N(c4ccccc4)c4ccccc4C)ccc4ccc1c2c43 ; Cc1cccc(N(c2ccccc2)c2ccc3ccc4c(N(c5ccccc5)c5cccc(C)c5)ccc5ccc2c3c54)c1 ; Cc1ccc(N(c2ccccc2)c2ccc3ccc4c(N(c5ccccc5)c5ccc(C)cc5)ccc5ccc2c3c54)cc1 ; CC(C)c1ccc(N(c2ccccc2)c2ccc3ccc4c(N(c5ccccc5)c5ccc(C(C)C)cc5)ccc5ccc2c3c54)cc1 ; CC(C)(C)c1ccc(N(c2ccccc2)c2ccc3ccc4c(N(c5ccccc5)c5ccc(C(C)(C)C)cc5)ccc5ccc2c3c54)cc1]

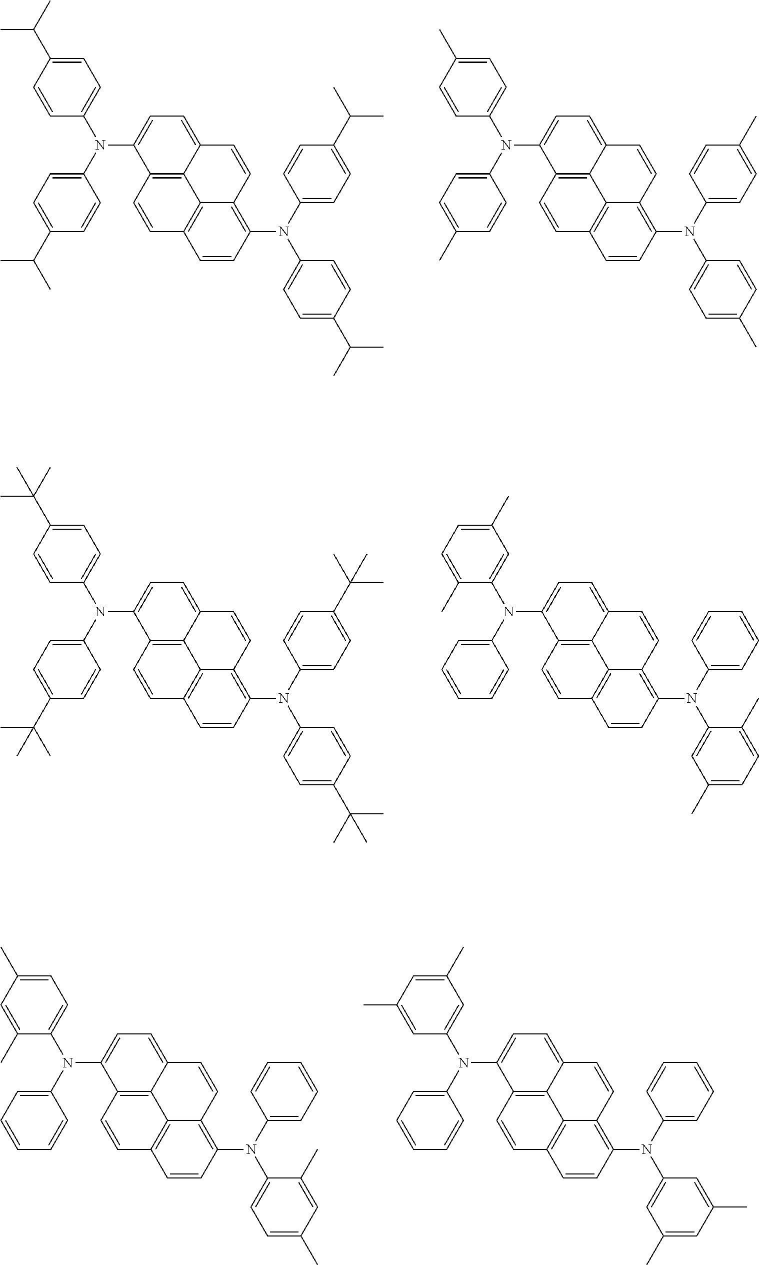

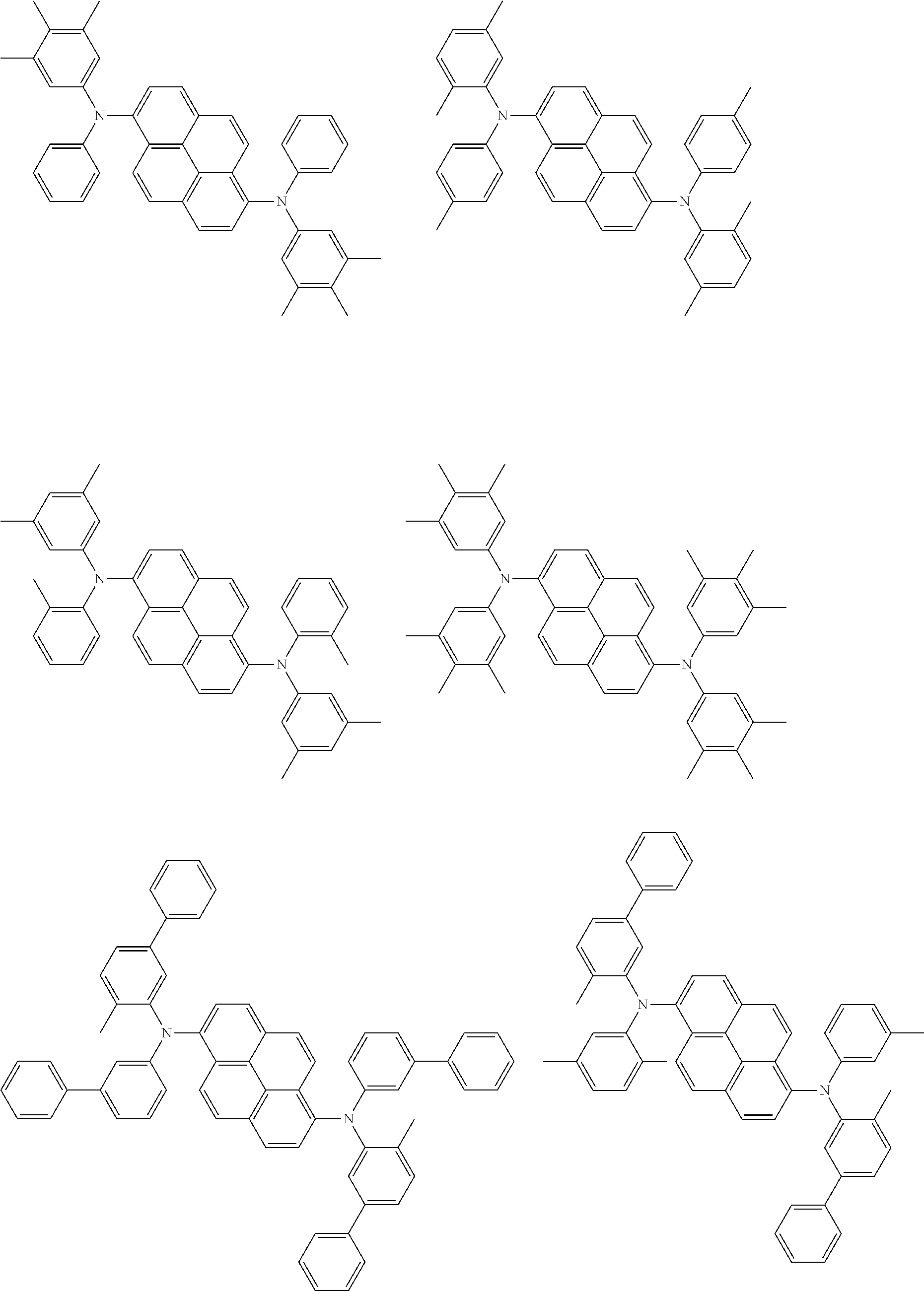

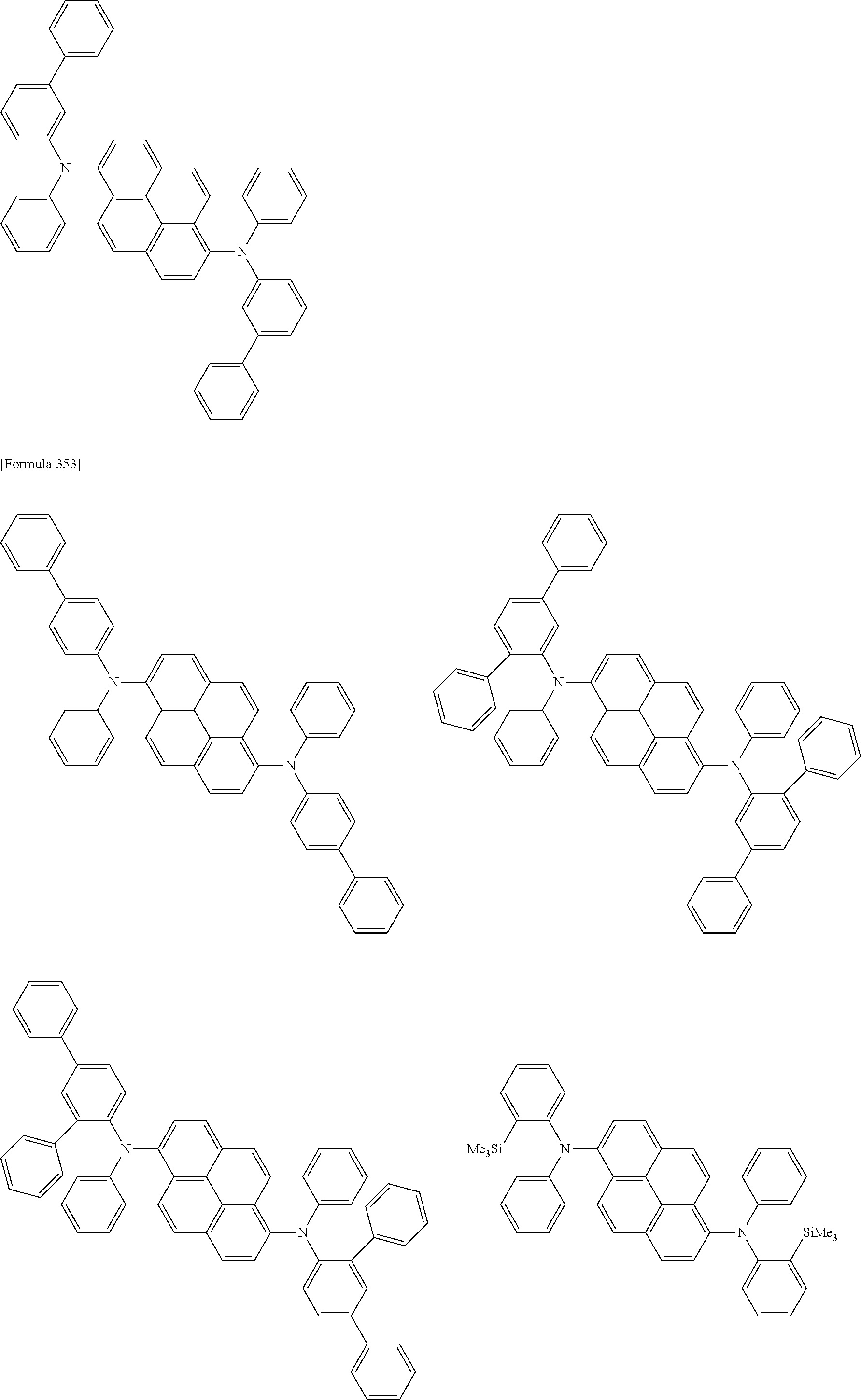 [SMILES: c1ccc(-c2cccc(N(c3ccccc3)c3ccc4ccc5c(N(c6ccccc6)c6cccc(-c7ccccc7)c6)ccc6ccc3c4c65)c2)cc1 ; c1ccc(-c2ccc(N(c3ccccc3)c3ccc4ccc5c(N(c6ccccc6)c6ccc(-c7ccccc7)cc6)ccc6ccc3c4c65)cc2)cc1 ; c1ccc(-c2ccc(-c3ccccc3)c(N(c3ccccc3)c3ccc4ccc5c(N(c6ccccc6)c6cc(-c7ccccc7)ccc6-c6ccccc6)ccc6ccc3c4c65)c2)cc1 ; c1ccc(-c2ccc(N(c3ccccc3)c3ccc4ccc5c(N(c6ccccc6)c6ccc(-c7ccccc7)cc6-c6ccccc6)ccc6ccc3c4c65)c(-c3ccccc3)c2)cc1 ; C[Si](C)(C)c1ccccc1N(c1ccccc1)c1ccc2ccc3c(N(c4ccccc4)c4ccccc4[Si](C)(C)C)ccc4ccc1c2c43]

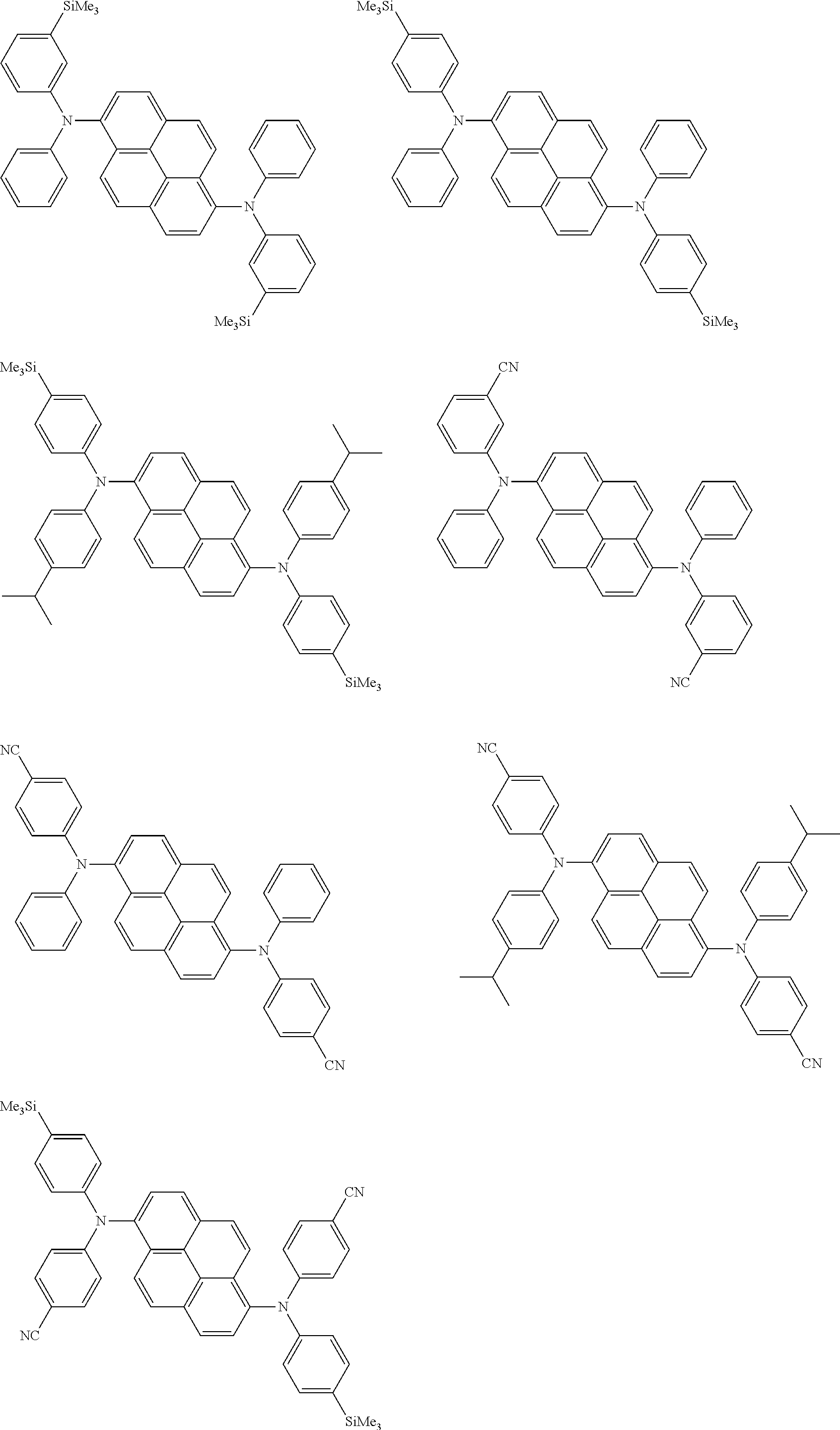

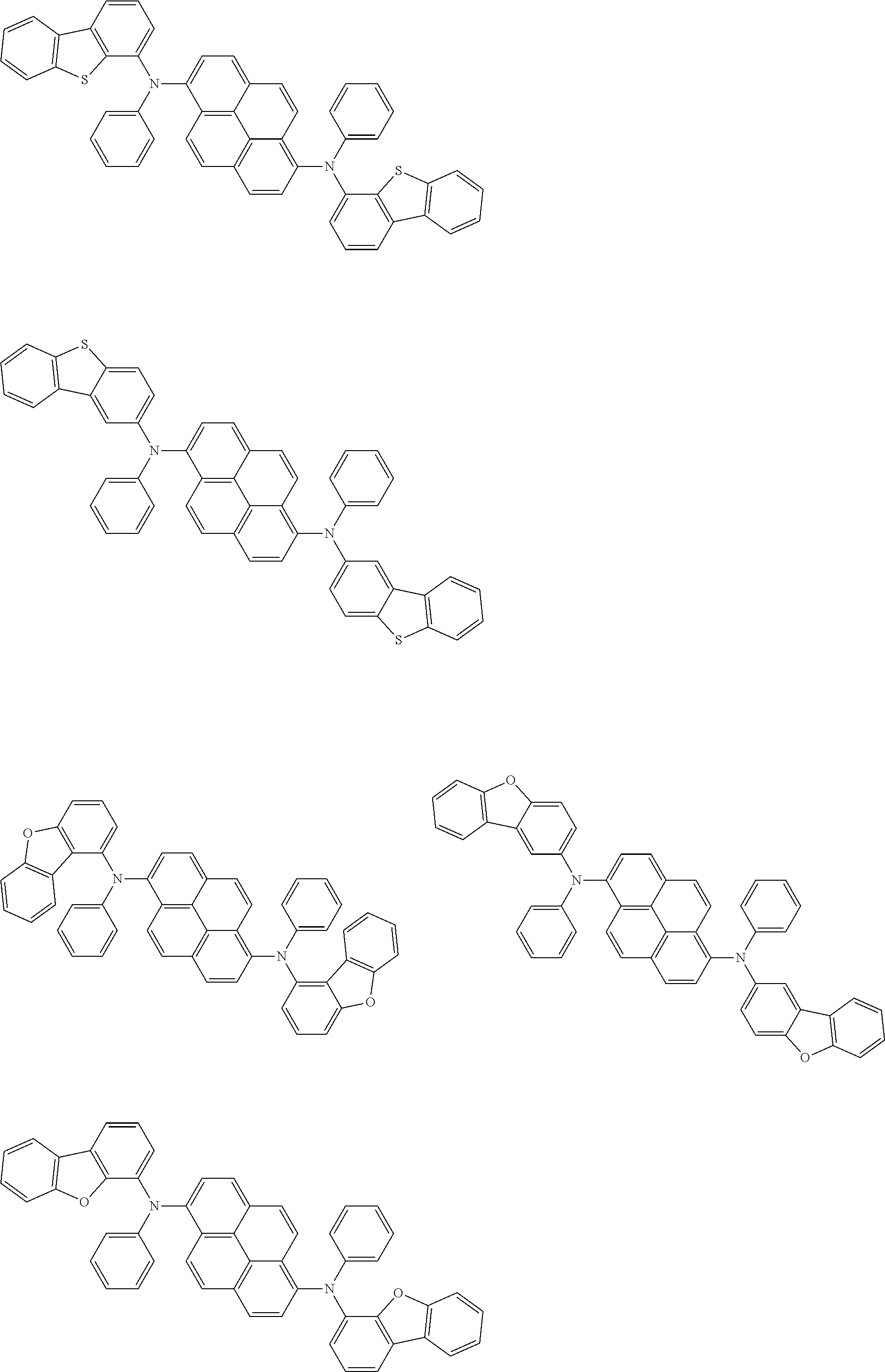 [SMILES: c1ccc(N(c2ccc3ccc4c(N(c5ccccc5)c5cccc6c5sc5ccccc56)ccc5ccc2c3c54)c2cccc3c2sc2ccccc23)cc1 ; c1ccc(N(c2ccc3sc4ccccc4c3c2)c2ccc3ccc4c(N(c5ccccc5)c5ccc6sc7ccccc7c6c5)ccc5ccc2c3c54)cc1 ; c1ccc(N(c2ccc3ccc4c(N(c5ccccc5)c5cccc6oc7ccccc7c56)ccc5ccc2c3c54)c2cccc3oc4ccccc4c23)cc1 ; c1ccc(N(c2ccc3oc4ccccc4c3c2)c2ccc3ccc4c(N(c5ccccc5)c5ccc6oc7ccccc7c6c5)ccc5ccc2c3c54)cc1 ; c1ccc(N(c2ccc3ccc4c(N(c5ccccc5)c5cccc6c5oc5ccccc56)ccc5ccc2c3c54)c2cccc3c2oc2ccccc23)cc1]

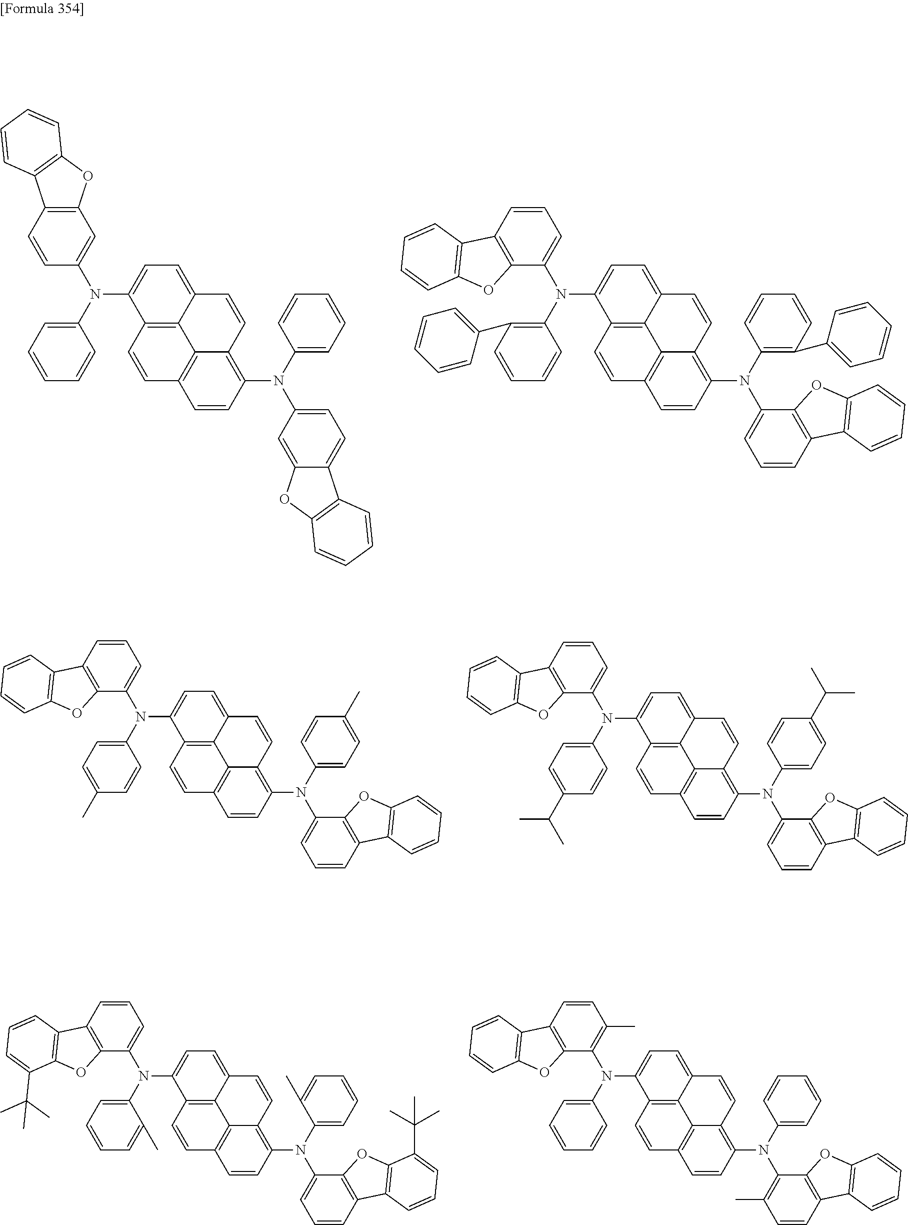

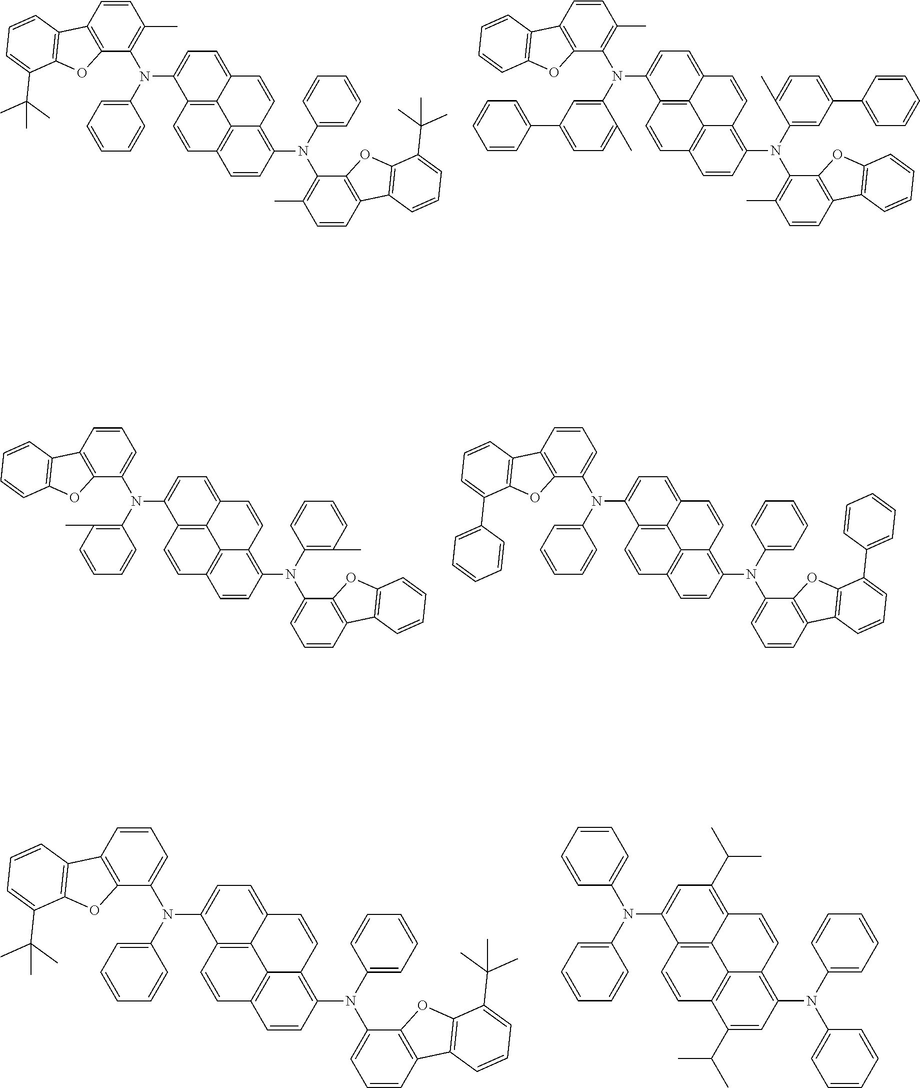

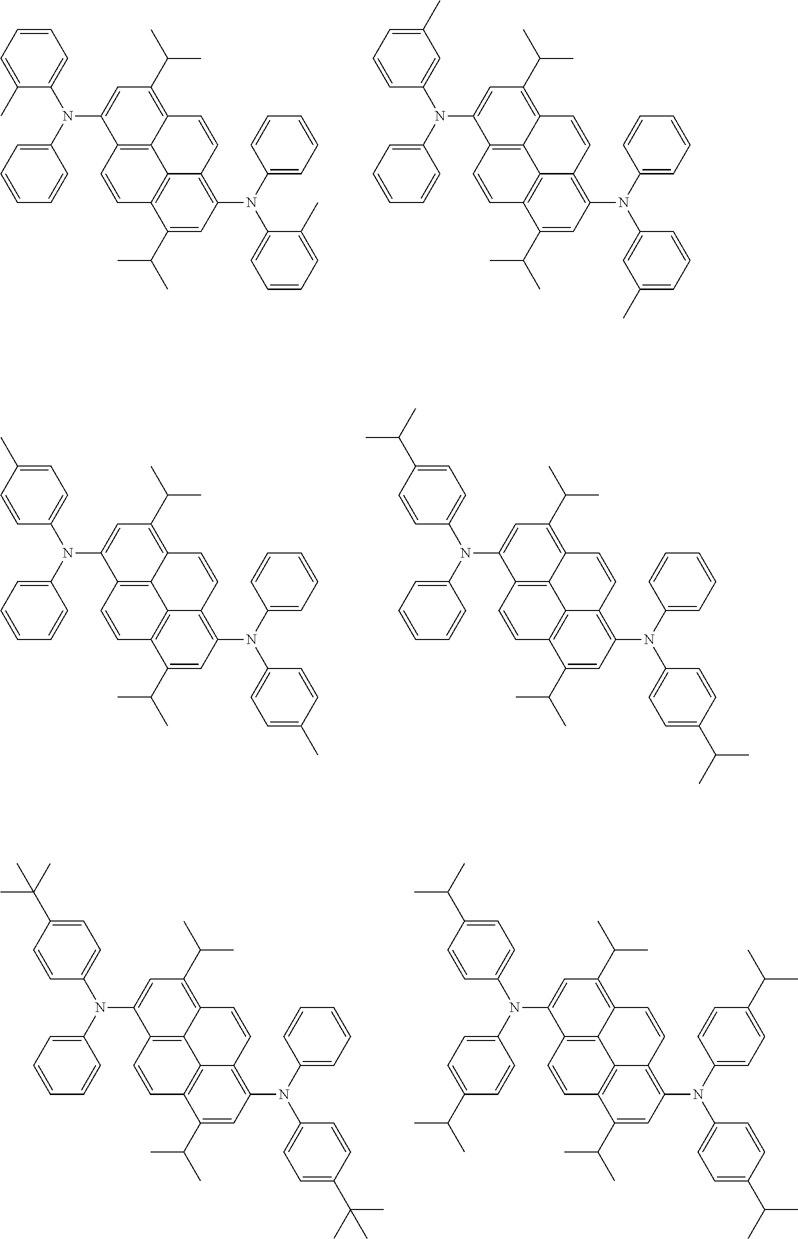

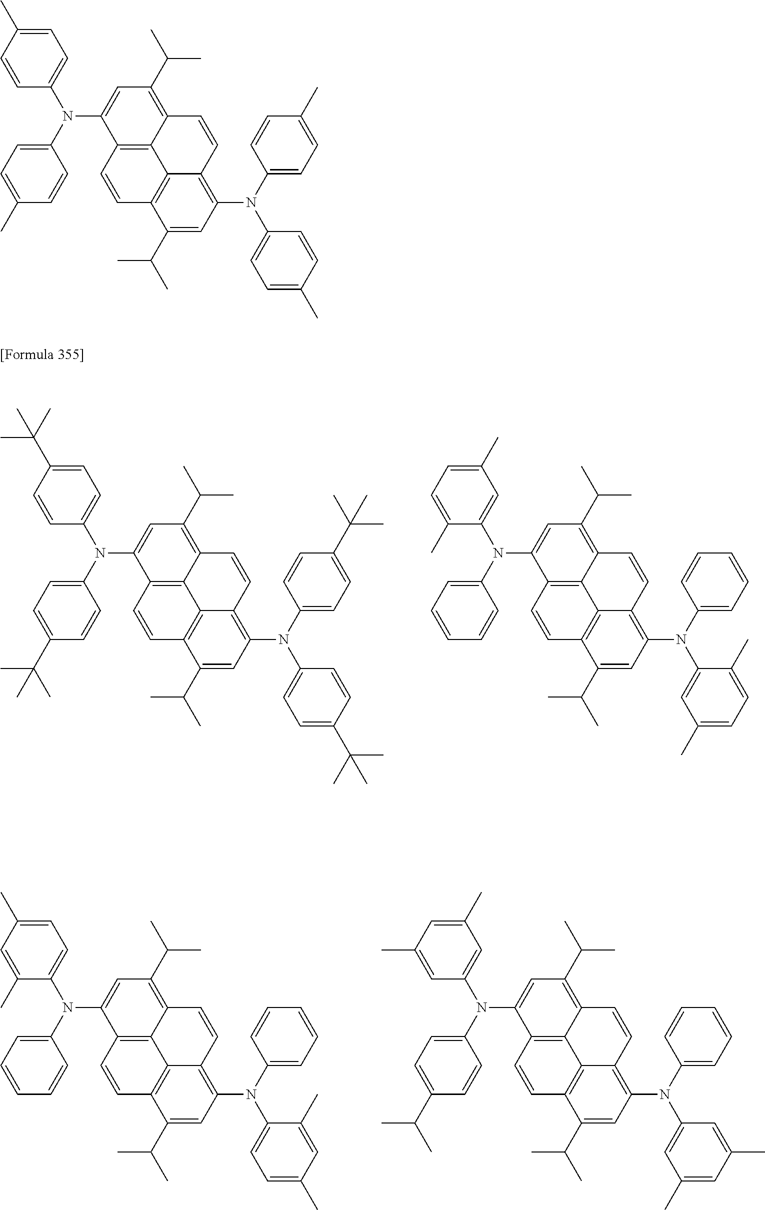 [SMILES: Cc1ccc(N(c2ccc(C)cc2)c2cc(C(C)C)c3ccc4c(N(c5ccc(C)cc5)c5ccc(C)cc5)cc(C(C)C)c5ccc2c3c54)cc1 ; CC(C)c1cc(N(c2ccc(C(C)(C)C)cc2)c2ccc(C(C)(C)C)cc2)c2ccc3c(C(C)C)cc(N(c4ccc(C(C)(C)C)cc4)c4ccc(C(C)(C)C)cc4)c4ccc1c2c34 ; Cc1ccc(C)c(N(c2ccccc2)c2cc(C(C)C)c3ccc4c(N(c5ccccc5)c5cc(C)ccc5C)cc(C(C)C)c5ccc2c3c54)c1 ; Cc1ccc(N(c2ccccc2)c2cc(C(C)C)c3ccc4c(N(c5ccccc5)c5ccc(C)cc5C)cc(C(C)C)c5ccc2c3c54)c(C)c1 ; Cc1cc(C)cc(N(c2ccccc2)c2cc(C(C)C)c3ccc4c(N(c5ccc(C(C)C)cc5)c5cc(C)cc(C)c5)cc(C(C)C)c5ccc2c3c54)c1]

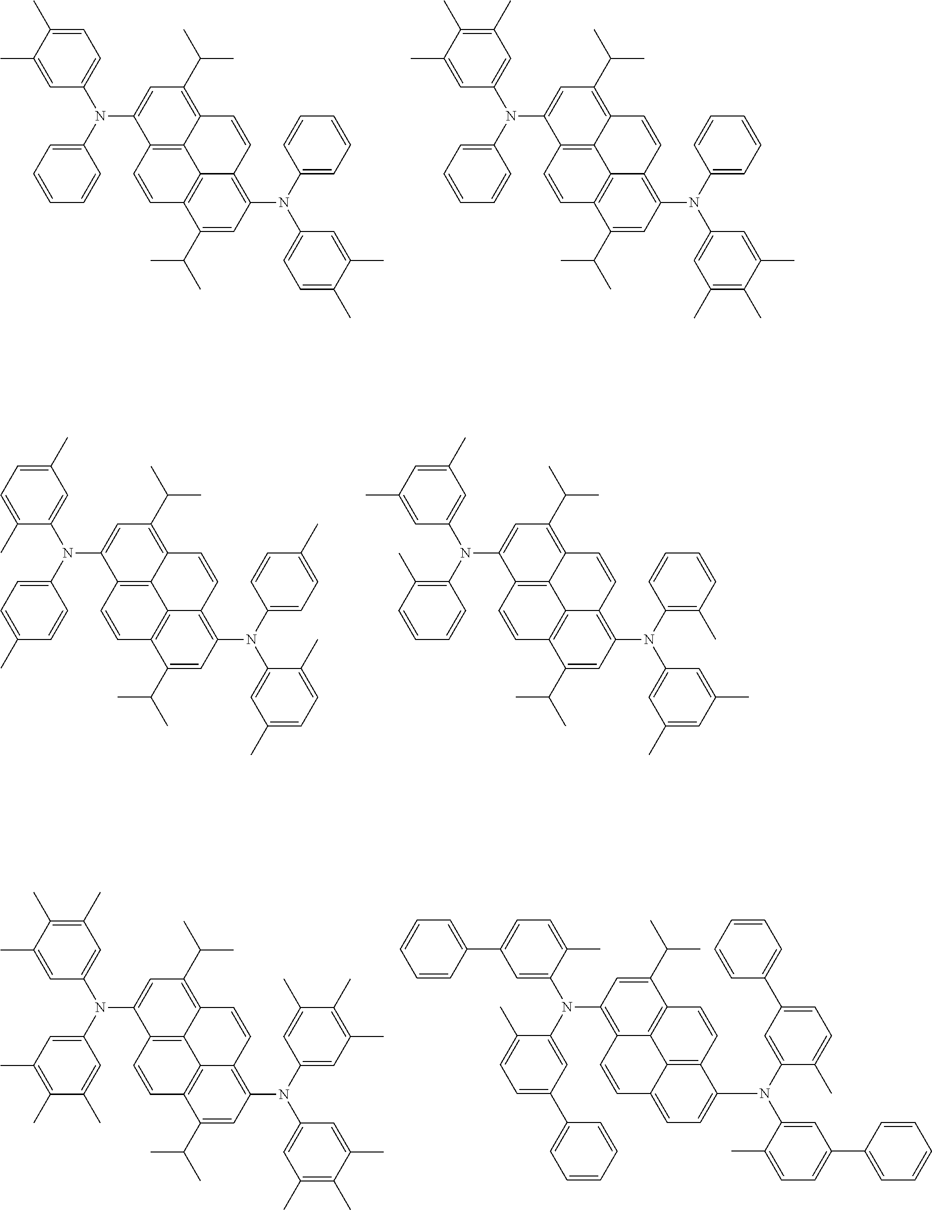

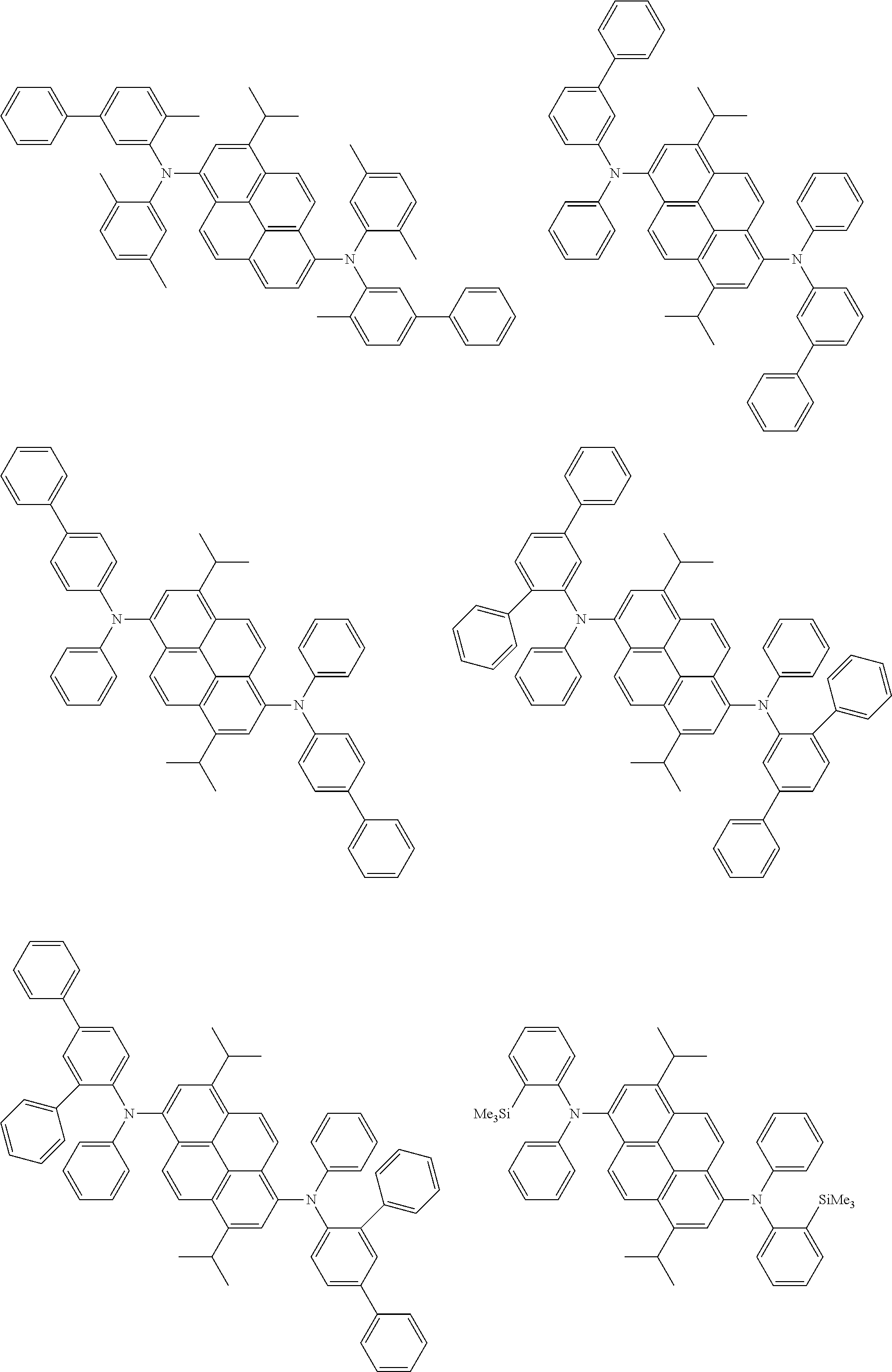

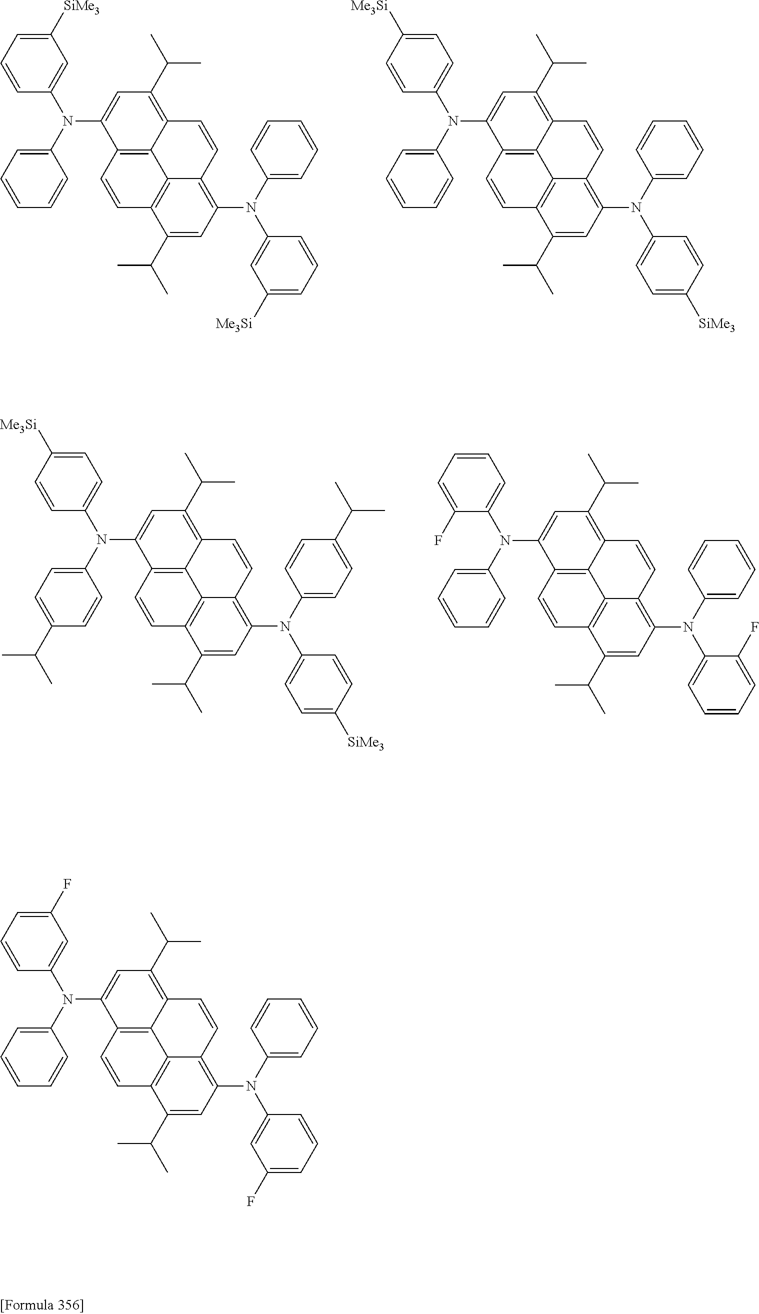

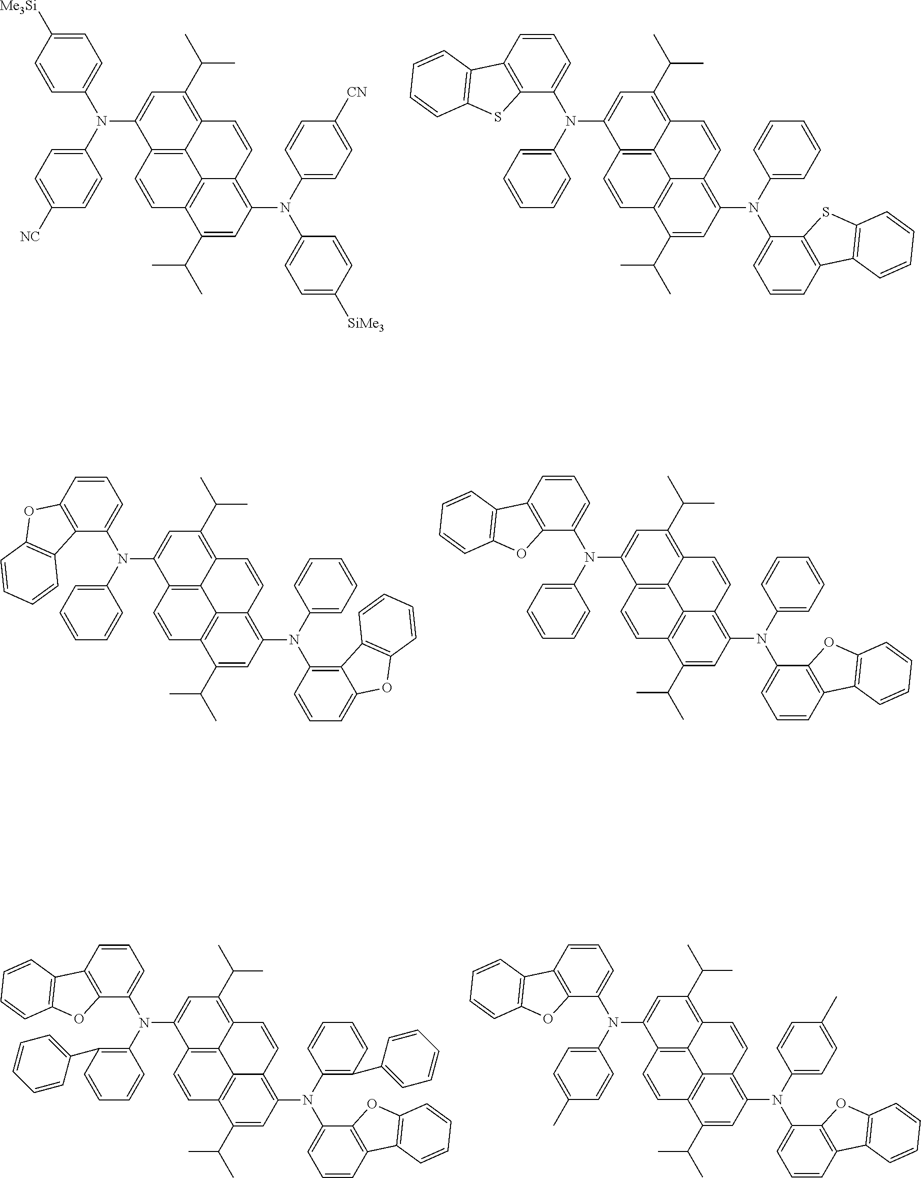

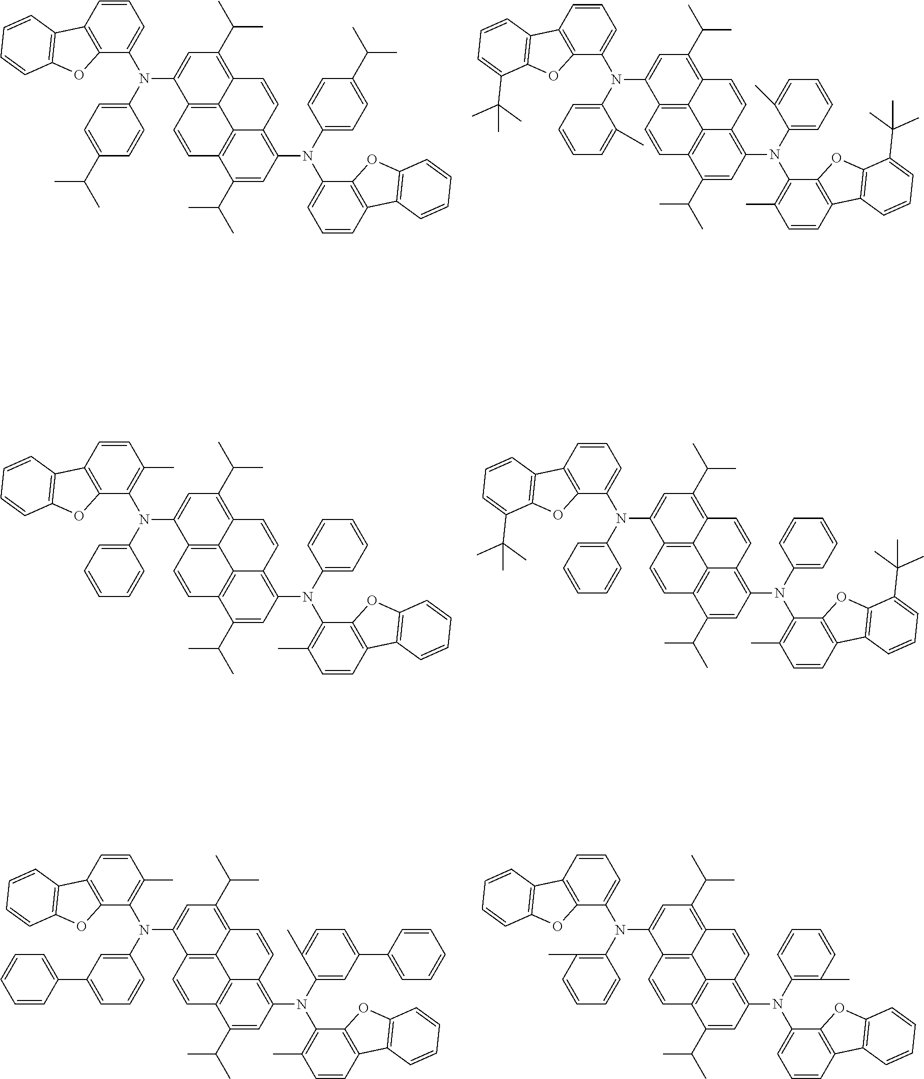

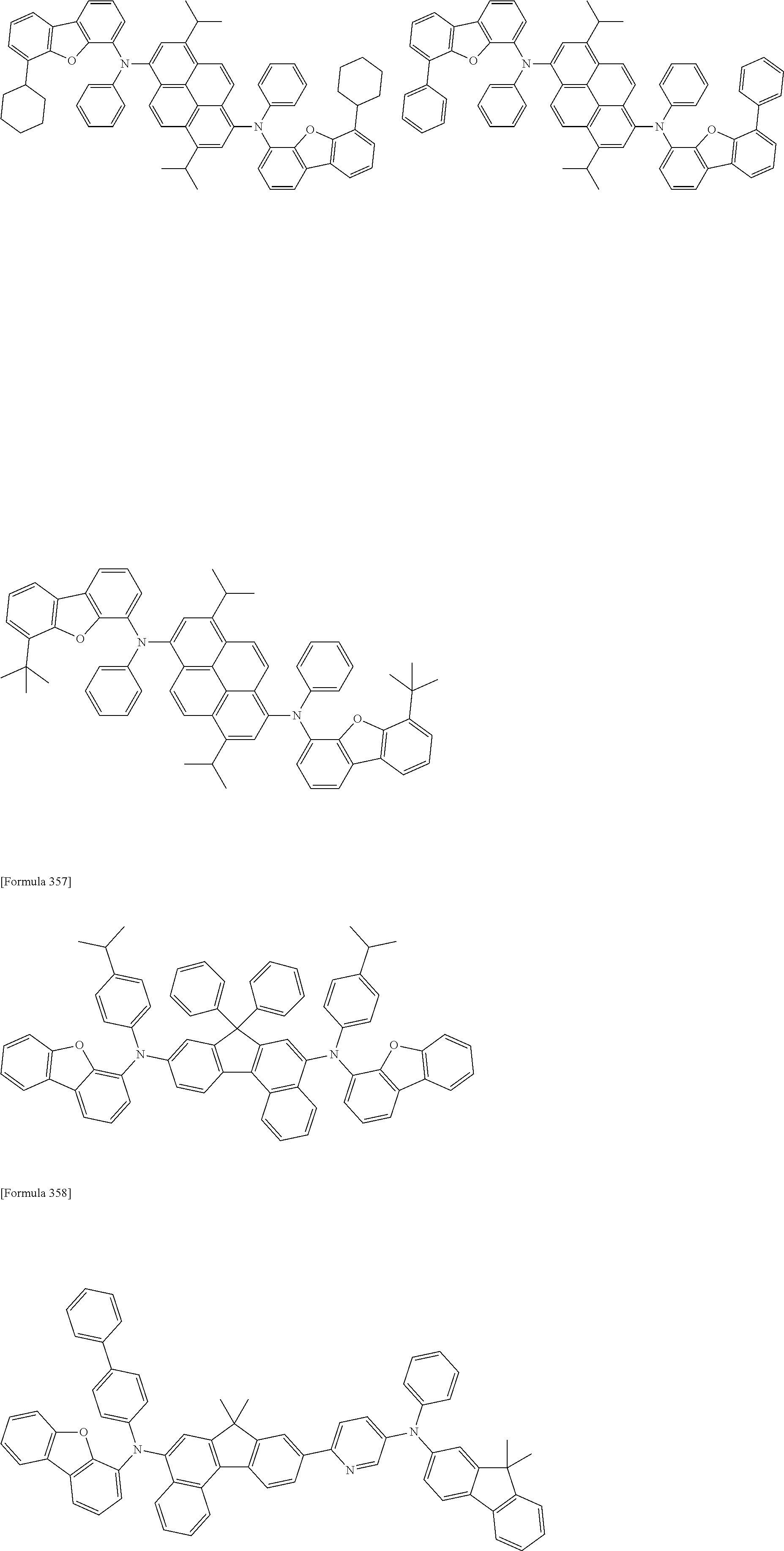

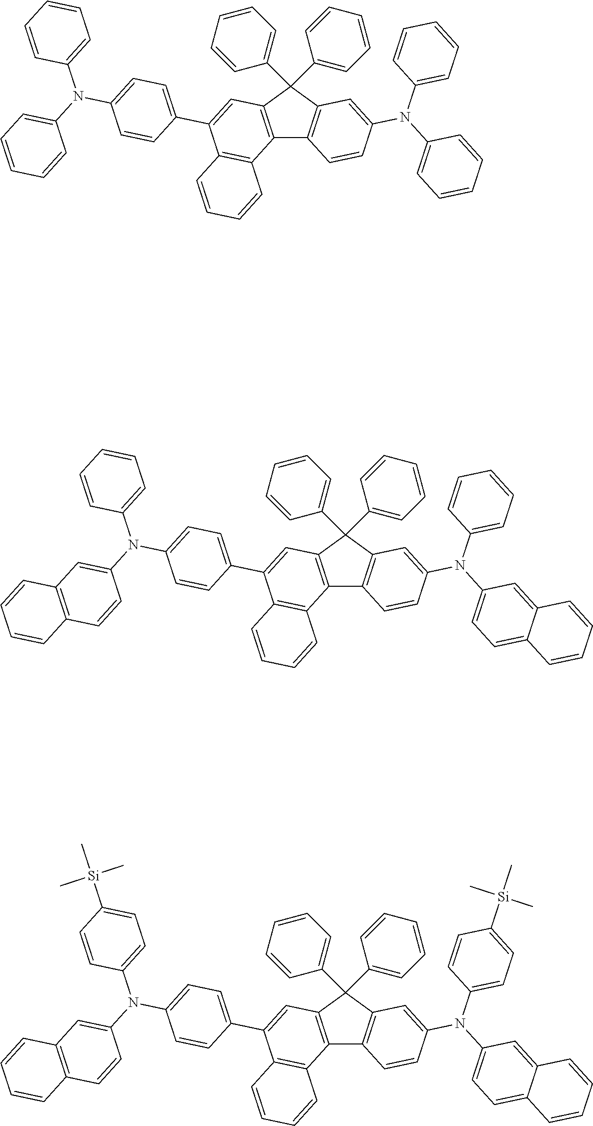 [SMILES: c1ccc(N(c2ccccc2)c2ccc(-c3cc4c(c5ccccc35)-c3ccc(N(c5ccccc5)c5ccccc5)cc3C4(c3ccccc3)c3ccccc3)cc2)cc1 ; c1ccc(N(c2ccc(-c3cc4c(c5ccccc35)-c3ccc(N(c5ccccc5)c5ccc6ccccc6c5)cc3C4(c3ccccc3)c3ccccc3)cc2)c2ccc3ccccc3c2)cc1 ; C[Si](C)(C)c1ccc(N(c2ccc(-c3cc4c(c5ccccc35)-c3ccc(N(c5ccc([Si](C)(C)C)cc5)c5ccc6ccccc6c5)cc3C4(c3ccccc3)c3ccccc3)cc2)c2ccc3ccccc3c2)cc1]

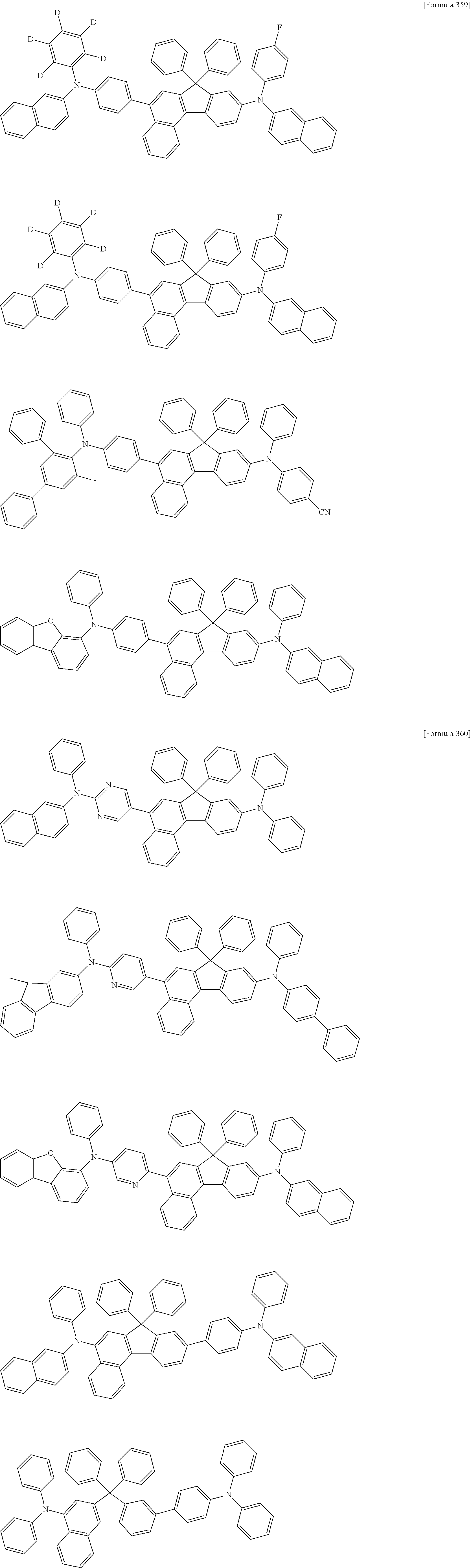

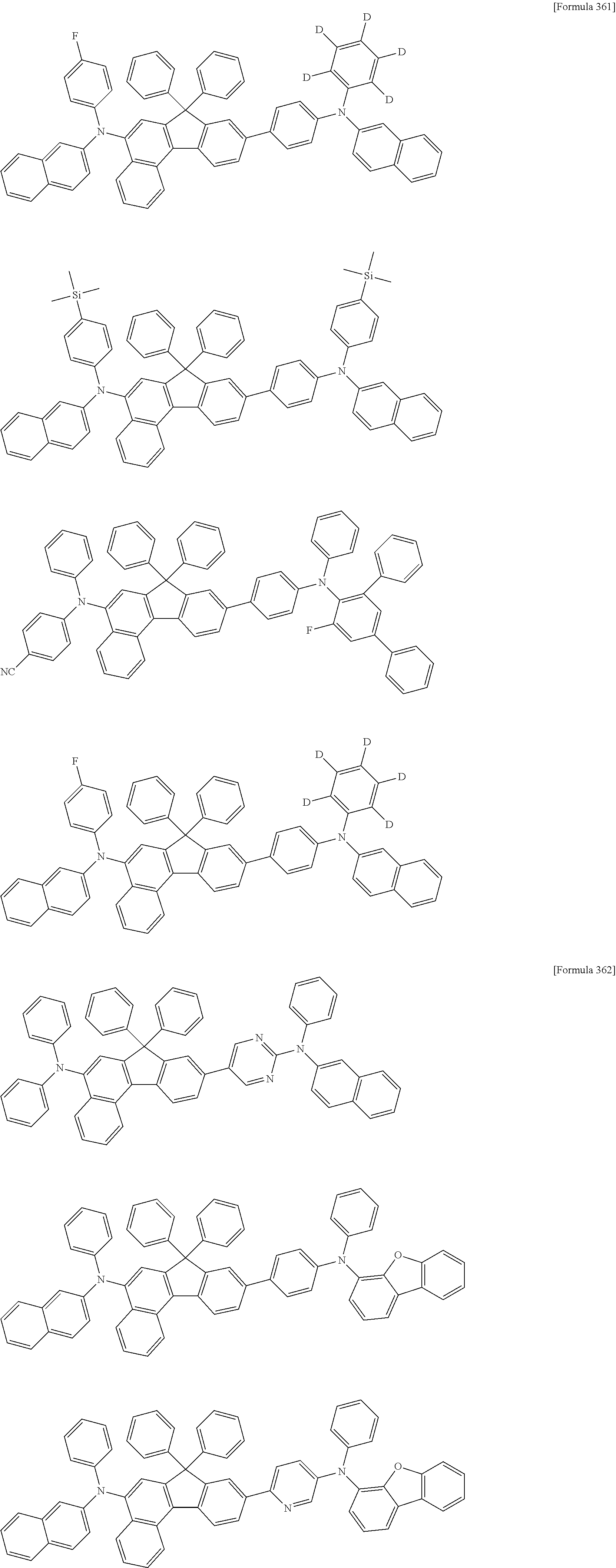

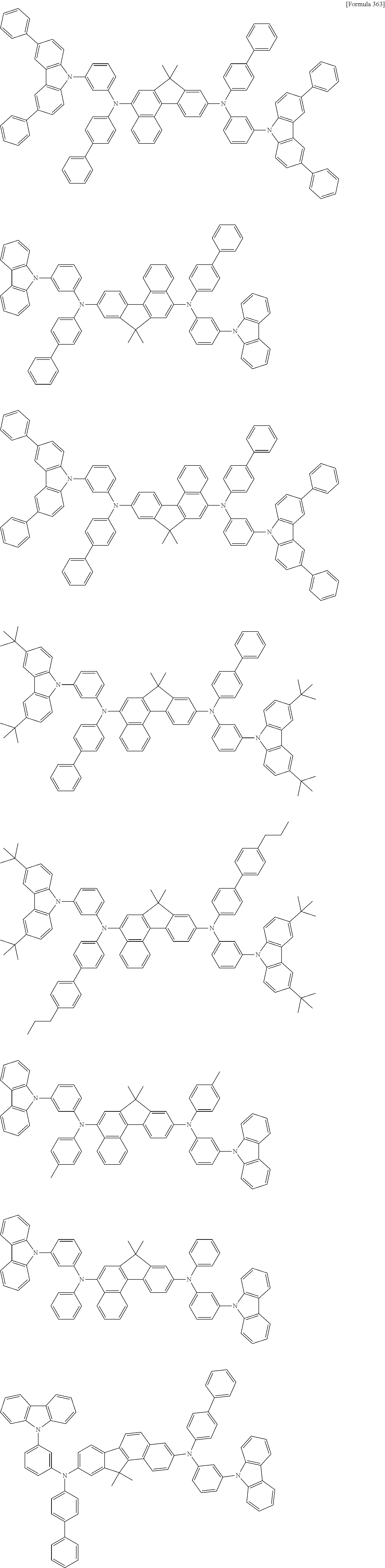

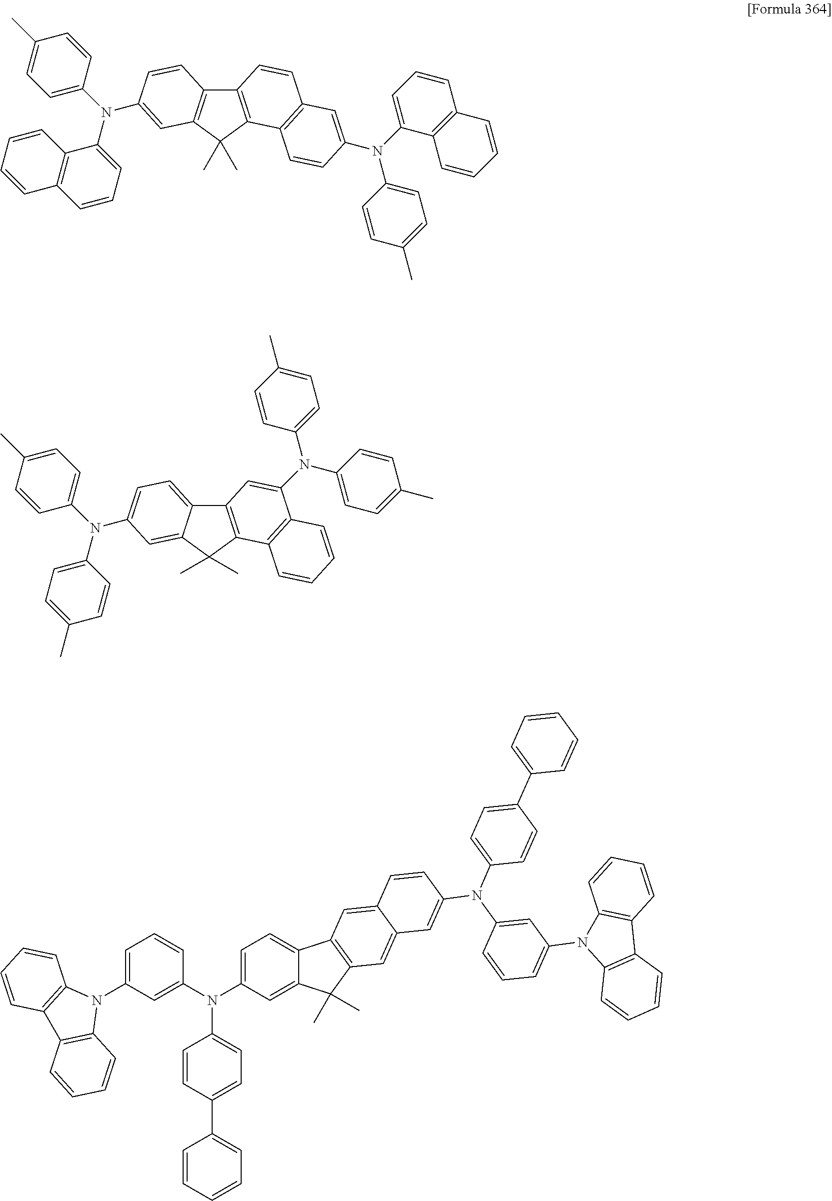

### Compound Represented by Formula (4)

The compound represented by the formula (4) will be described below.

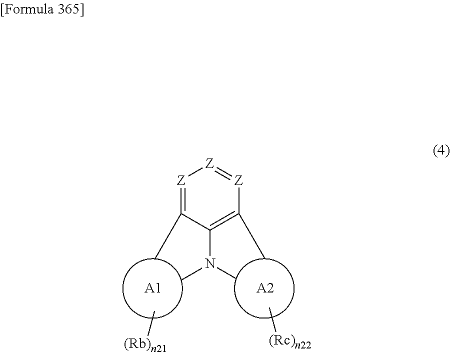

In the formula (4):
Z are each independently CRa or a nitrogen atom;
a ring A1 and a ring A2 are each independently a substituted or unsubstituted aromatic hydrocarbon ring having 6 to 50 ring carbon atoms, or a substituted or unsubstituted heterocycle having 5 to 50 ring atoms;
when a plurality of Ra are present, at least one combination of adjacent two or more of the plurality of Ra are mutually bonded to form a substituted or unsubstituted monocyclic ring, mutually bonded to form a substituted or unsubstituted fused ring, or not mutually bonded;
n21 and n22 are each independently 0, 1, 2, 3, or 4;
when a plurality of Rb are present, at least one combination of adjacent two or more of the plurality of Rb are mutually bonded to form a substituted or unsubstituted monocyclic ring, mutually bonded to form a substituted or unsubstituted fused ring, or not mutually bonded;
when a plurality of Rc are present, at least one combination of adjacent two or more of the plurality of Rc are mutually bonded to form a substituted or unsubstituted monocyclic ring, mutually bonded to form a substituted or unsubstituted fused ring, or not mutually bonded; and
Ra, Rb and Rc forming neither the monocyclic ring nor the fused ring are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

The “aromatic hydrocarbon ring” for the ring A1 and the ring A2 has the same structure as a compound formed by introducing a hydrogen atom to the “aryl group” described above.

Ring atoms of the “aromatic hydrocarbon ring” for the ring A1 and the ring A2 include two carbon atoms on a fused bicyclic structure at the center of the formula (4).

Specific examples of the “substituted or unsubstituted aromatic hydrocarbon ring having 6 to 50 ring carbon atoms” include a compound formed by introducing a hydrogen atom to the “aryl group” described in the specific example group G1.

The “heterocycle” for the ring A1 and the ring A2 has the same structure as a compound formed by introducing a hydrogen atom to the “heterocyclic group” described above.

Ring atoms of the “heterocycle” for the ring A1 and the ring A2 include two carbon atoms on a fused bicyclic structure at the center of the formula (4).

Specific examples of the “substituted or unsubstituted heterocycle having 5 to 50 ring atoms” include a compound formed by introducing a hydrogen atom to the “heterocyclic group” described in the specific example group G2.

Rb is bonded to any one of carbon atoms forming the aromatic hydrocarbon ring as the ring A1 or any one of atoms forming the heterocycle as the ring A1.

Rc is bonded to any one of carbon atoms forming the aromatic hydrocarbon ring as the ring A2 or any one of atoms forming the heterocycle as the ring A2.

At least one of Ra, Rb, or Rc is preferably a group represented by a formula (4a) below. More preferably, at least two of Ra, Rb, or Rc are each a group represented by the formula (4a).

[Formula 366]

*-L₄₀₁-Ar₄₀₁ (4a)

In the formula (4a):
L₄₀₁ is a single bond, a substituted or unsubstituted arylene group having 6 to 30 ring carbon atoms, or a substituted or unsubstituted divalent heterocyclic group having 5 to 30 ring atoms; and
A₄₀₁ is a substituted or unsubstituted aryl group having 6 to 50 ring carbon atoms, a substituted or unsubstituted heterocyclic group having 5 to 50 ring atoms, or a group represented by a formula (4b) below.

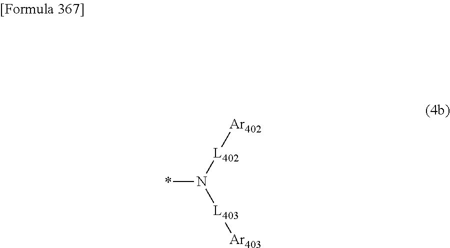

In the formula (4b):
L₄₀₂ and L₄₀₃ are each independently a single bond, a substituted or unsubstituted arylene group having 6 to 30 ring carbon atoms, or a substituted or unsubstituted divalent heterocyclic group having 5 to 30 ring atoms;
a combination of R₄₀₂ and R₄₀₃ are mutually bonded to form a substituted or unsubstituted monocyclic ring, mutually bonded to form a substituted or unsubstituted fused ring, or not mutually bonded; and
Ar₄₀₂ and Ar₄₀₃ forming neither the monocyclic ring nor the fused ring are each independently a substituted or unsubstituted aryl group having 6 to 50 ring carbon atoms, or a substituted or unsubstituted heterocyclic group having 5 to 50 ring atoms.

In an exemplary embodiment, the compound represented by the formula (4) is represented by a formula (42) below.

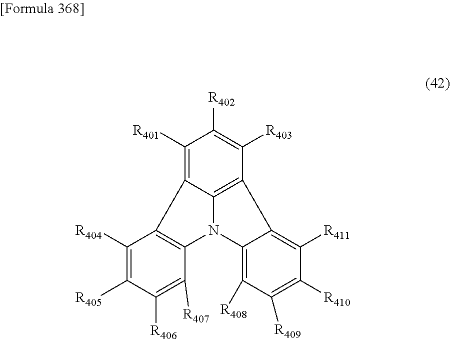

In the formula (42):
at least one combination of adjacent two or more of R₄₀₁ to R₄₁₁ are mutually bonded to form a substituted or unsubstituted monocyclic ring, mutually bonded to form a substituted or unsubstituted fused ring, or not mutually bonded; and
R₄₀₁ to R₄₁₁ forming neither the monocyclic ring nor the fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

Preferably, at least one of R₄₀₁ to R₄₁₁ is a group represented by the formula (4a). More preferably, at least two of R₄₀₁ to R₄₁₁ are each a group represented by the formula (4a).

R₄₀₄ and R₄₁₁ are each preferably a group represented by the formula (4a).

In an exemplary embodiment, the compound represented by the formula (4) is a compound formed by bonding a structure represented by a formula (4-1) or a formula (4-2) below to the ring A1.

Further, in an exemplary embodiment, the compound represented by the formula (42) is a compound formed by bonding a structure represented by the formula (4-1) or the formula (4-2) to a ring bonded to R₄₀₄ to R₄₀₇.

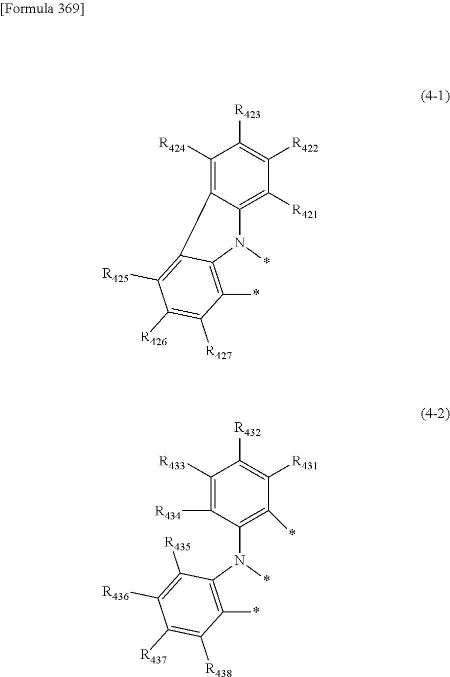

In the formula (4-1), two * are each independently bonded to a ring carbon atom of the aromatic hydrocarbon ring or a ring atom of the heterocycle as the A1 ring in the formula (4) or bonded to one of R₄₀₄ to R₄₀₇ in the formula (42);
in the formula (4-2), three * are each independently bonded to a ring carbon atom of the aromatic hydrocarbon ring or a ring atom of the heterocycle as the A1 ring in the formula (4) or bonded to one of R₄₀₄ to R₄₀₇ in the formula (42);
at least one combination of adjacent two or more of R₄₂₁ to R₄₂₇ are mutually bonded to form a substituted or unsubstituted monocyclic ring, mutually bonded to form a substituted or unsubstituted fused ring, or not mutually bonded;
at least one combination of adjacent two or more of R₄₃₁ to R₄₃₈ are mutually bonded to form a substituted or unsubstituted monocyclic ring, mutually bonded to form a substituted or unsubstituted fused ring, or not mutually bonded; and
R₄₂₁ to R₄₂₇ and R₄₃₁ to R₄₃₈ forming neither the monocyclic ring nor the fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In an exemplary embodiment, the compound represented by the formula (4) is a compound represented by a formula (41-3), (41-4), or (41-5) below.

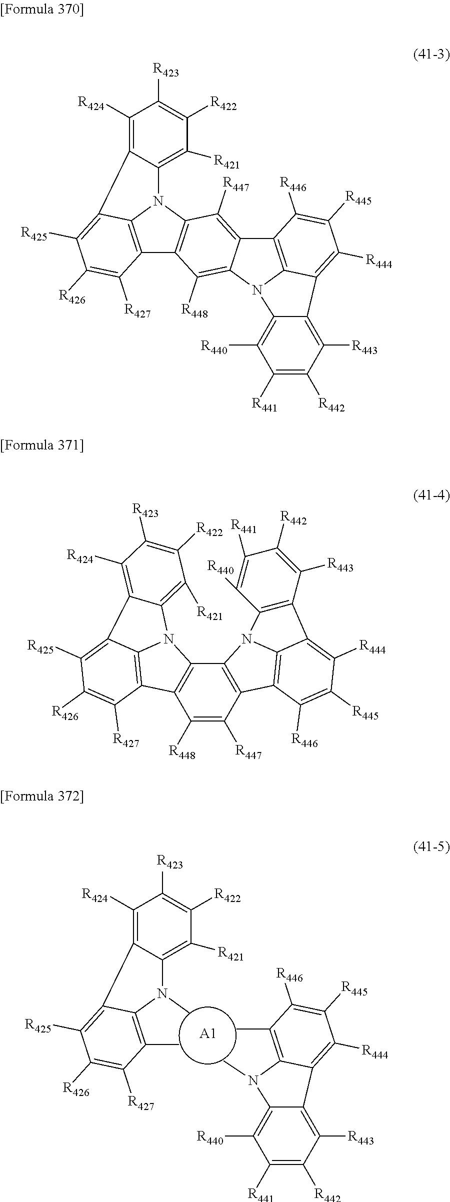

In the formulae (41-3), (41-4), and (41-5):
a ring A1 is as defined in the formula (4);
R₄₂₁ to R₄₂₇ each independently represent the same as R₄₂₁ to R₄₂₇ in the formula (4-1); and
R₄₄₀ to R₄₄₈ each independently represent the same as R₄₀₁ to R₄₁₁ in the formula (42).

In an exemplary embodiment, a substituted or unsubstituted aromatic hydrocarbon ring having 6 to 50 ring carbon atoms as the ring A1 in the formula (41-5) is a substituted or unsubstituted naphthalene ring, or a substituted or unsubstituted fluorene ring.

In an exemplary embodiment, a substituted or unsubstituted heterocycle having 5 to 50 ring atoms as the ring A1 in the formula (41-5) is a substituted or unsubstituted dibenzofuran ring, a substituted or unsubstituted carbazole ring, or a substituted or unsubstituted dibenzothiophene ring.

In an exemplary embodiment, the compound represented by the formula (4) or the formula (42) is selected from the group consisting of compounds represented by formulae (461) to (467) below.

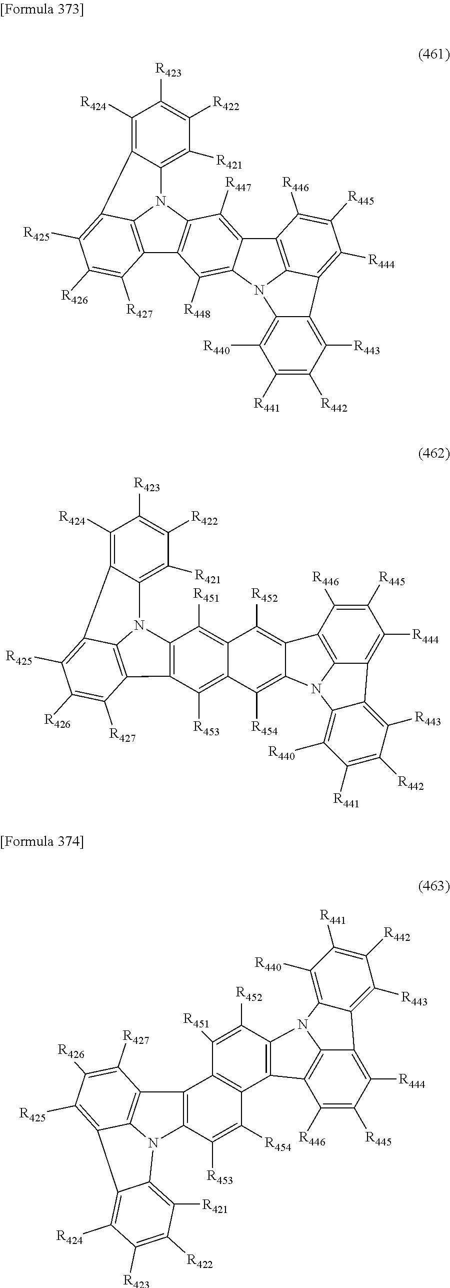

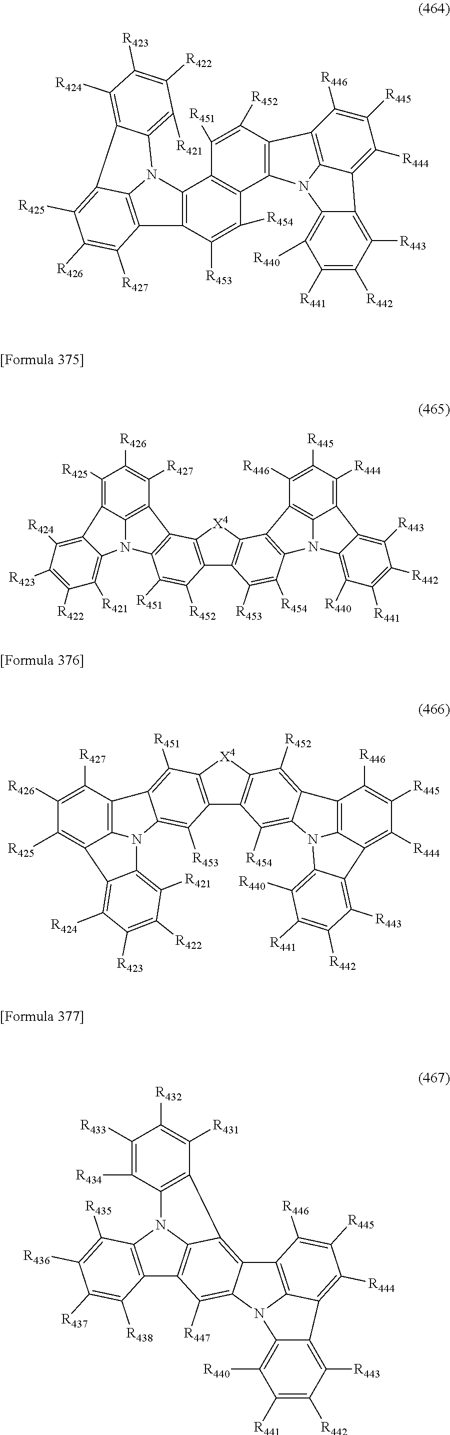

In the formulae (461), (462), (463), (464), (465), (466) and (467):
R₄₂₁ to R₄₂₇ each independently represent the same as R₄₂₁ to R₄₂₇ in the formula (4-1);
R₄₃₁ to R₄₃₈ each independently represent the same as R₄₃₁ to R₄₃₈ in the formula (4-2);
R₄₄₀ to R₄₄₈ and R₄₅₁ to R₄₅₄ each independently represent the same as R₄₀₁ to R₄₁₁ in the formula (42);
X₄ is an oxygen atom, N(R₈₀₁), or C(R₈₀₂)(R₈₀₃);
R₈₀₁, R₈₀₂ and R₈₀₃ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
a substituted or unsubstituted alkyl group having 1 to 50 carbon atoms, or a substituted or unsubstituted aryl group having 6 to 50 ring carbon atoms;
when a plurality of R₈₀₁ are present, the plurality of R₈₀₁ are mutually the same or different;
when a plurality of R₈₀₂ are present, the plurality of R₈₀₂ are mutually the same or different; and
when a plurality of R₈₀₃ are present, the plurality of R₈₀₃ are mutually the same or different.

In the compound represented by the formula (42) in an exemplary embodiment, at least one combination of adjacent two or more of R₄₀₁ to R₄₁₁ are mutually bonded to form a substituted or unsubstituted monocyclic ring or are mutually bonded to form a substituted or unsubstituted fused ring. The compound represented by the formula (42) in the exemplary embodiment is described in detail as a compound represented by a formula (45) below.

### Compound Represented by Formula (45)

The compound represented by the formula (45) will be described below.

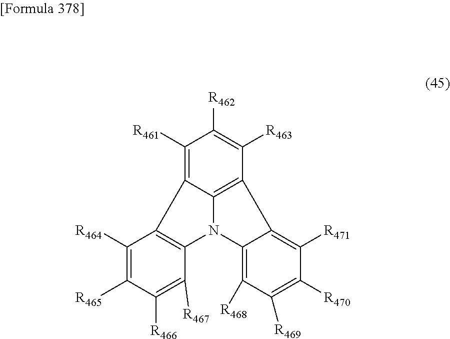

In the formula (45), two or more of combinations selected from the group consisting of a combination of R₄₆₁ and R₄₆₂, a combination of R₄₆₂ and R₄₆₃, a combination of R₄₆₄ and R₄₆₅, a combination of R₄₆₅ and R₄₆₆, a combination of R₄₆₆ and R₄₆₇, a combination of R₄₆₈ and R₄₆₉, a combination of R₄₆₉ and R₄₇₀, and a combination of R₄₇₀ and R₄₇₁ are mutually bonded to form a substituted or unsubstituted monocyclic ring or a substituted or unsubstituted fused ring.

Here, the combination of R₄₆₁ and R₄₆₂ and the combination of R₄₆₂ and R₄₆₃; the combination of R₄₆₄ and R₄₆₅ and the combination of R₄₆₅ and R₄₆₆; the combination of R₄₆₅ and R₄₆₆ and the combination of R₄₆₆ and R₄₆₇; the combination of R₄₆₈ and R₄₆₉ and the combination of R₄₆₉ and R₄₇₀; and the combination of R₄₆₉ and R₄₇₀ and the combination of R₄₇₀ and R₄₇₁ do not simultaneously form a ring;
the two or more rings formed by R₄₆₁ to R₄₇₁ are mutually the same or different; and
R₄₆₁ to R₄₇₁ forming neither the monocyclic ring nor the fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In the formula (45), Rₙ and Rₙ₊₁ (n being an integer selected from 461, 462, 464 to 466, and 468 to 470) are mutually bonded to form a substituted or unsubstituted monocyclic ring or fused ring together with two ring carbon atoms bonded to Rₙ and Rₙ₊₁. The ring is preferably formed of atoms selected from the group consisting of a carbon atom, an oxygen atom, a sulfur atom, and a nitrogen atom, and is preferably formed of 3 to 7 atoms, more preferably 5 or 6 atoms.

The number of the above cyclic structures in the compound represented by the formula (45) is, for instance, 2, 3, or 4. The two or more of the cyclic structures may be present on the same benzene ring on the basic skeleton represented by the formula (45) or may be present on different benzene rings. For instance, when three cyclic structures are present, each of the cyclic structures may be present on the corresponding one of the three benzene rings of the formula (45).

The above cyclic structures in the compound represented by the formula (45) are exemplified by structures represented by formulae (451) to (460) below.

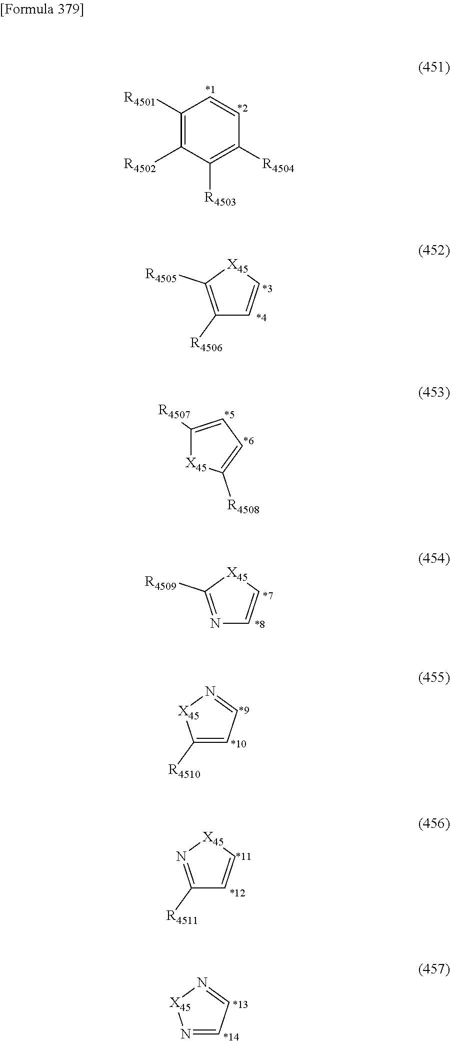

In the formulae (451) to (457):
each combination of *1 and *2, *3 and *4, *5 and *6, *7 and *8, *9 and *10, * 11 and *12, and *13 and *14 represent the two ring carbon atoms bonded to Rₙ and Rₙ₊₁;
the ring carbon atom bonded to Rₙ may be any one of the two ring carbon atoms represented by *1 and *2, *3 and *4, *5 and *6, *7 and *8, *9 and *10, *11 and *12, and *13 and *14;
X₄₅ is C(R₄₅₁₂)(R₄₅₁₃), NR₄₅₁₄, an oxygen atom, or a sulfur atom;
at least one combination of adjacent two or more of R₄₅₀₁ to R₄₅₀₆ and R₄₅₁₂ to R₄₅₁₃ are mutually bonded to form a substituted or unsubstituted monocyclic ring, mutually bonded to form a substituted or unsubstituted fused ring, or not mutually bonded; and
R₄₅₀₁ to R₄₅₁₄ forming neither the monocyclic ring nor the fused ring each independently represent the same as R₄₆₁ to R₄₇₁ in the formula (45).

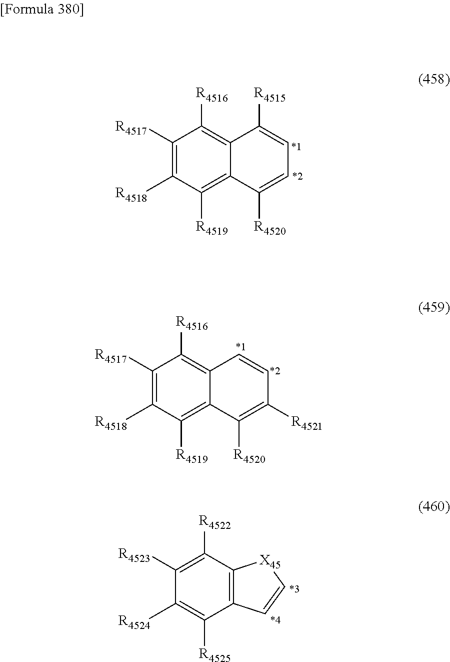

In the formulae (458) to (460):
each combination of *1 and *2, and *3 and *4 represent the two ring carbon atoms bonded to Rₙ and Rₙ₊₁;
the ring carbon atom bonded to Rₙ may be any one of the two ring carbon atoms represented by *1 and *2, or *3 and *4;
X₄₅ is C(R₄₅₁₂)(R₄₅₁₃), NR₄₅₁₄, an oxygen atom, or a sulfur atom;
at least one combination of adjacent two or more of R₄₅₁₂ to R₄₅₁₃ and R₄₅₁₅ to R₄₅₂₅ are mutually bonded to form a substituted or unsubstituted monocyclic ring, mutually bonded to form a substituted or unsubstituted fused ring, or not mutually bonded; and
R₄₅₁₂ to R₄₅₁₃, R₄₅₁₅ to R₄₅₂₁ and R₄₅₂₂ to R₄₅₂₅ forming neither the monocyclic ring nor the fused ring, and R₄₅₁₄ each independently represent the same as R₄₆₁ to R₄₇₁ in the formula (45).

In the formula (45), it is preferable that at least one of R₄₆₂, R₄₆₄, R₄₆₅, R₄₇₀ or R₄₇₁ (preferably, at least one of R₄₆₂, R₄₆₅ or R₄₇₀, more preferably R₄₆₂) is a group forming no cyclic structure.

(i) In the formula (45), a substituent, if present, for a cyclic structure formed by Rₙ and Rₙ₊₁, (ii) in the formula (45), R₄₆₁ to R₄₇₁ forming no cyclic structure, and (iii) R₄₅₀₁ to R₄₅₁₄, R₄₅₁₅ to R₄₅₂₅ in the formulae (451) to (460) are preferably each independently a group selected from the group consisting of a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —N(R₉₀₆)(R₉₀₇), a substituted or unsubstituted aryl group having 6 to 50 ring carbon atoms, a substituted or unsubstituted heterocyclic group having 5 to 50 ring atoms, or groups represented by formulae (461) to (464) below.

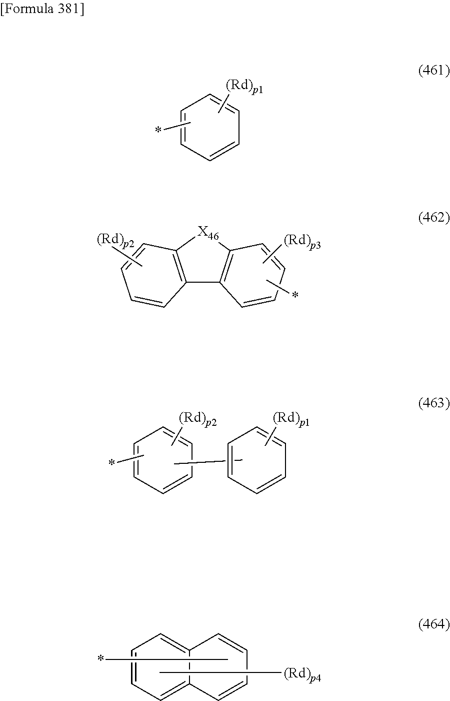

In the formulae (461) to (464):
R_{d} are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms;
X₄₆ is C(R₈₀₁)(R₈₀₂), NR₈₀₃, an oxygen atom, or a sulfur atom;
R₈₀₁, R₈₀₂ and R₈₀₃ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms; a substituted or unsubstituted alkyl group having 1 to 50 carbon atoms, or a substituted or unsubstituted aryl group having 6 to 50 ring carbon atoms;
when a plurality of R₈₀₁ are present, the plurality of R₈₀₁ are mutually the same or different;
when a plurality of R₈₀₂ are present, the plurality of R₈₀₂ are mutually the same or different;
when a plurality of R₈₀₃ are present, the plurality of R₈₀₃ are mutually the same or different;
p1 is 5;
p2 is 4;
p3 is 3;
p4 is 7; and
* in the formulae (461) to (464) each independently represent a bonding position to a cyclic structure.

In the third compound and the fourth compound, R₉₀₁ to R₉₀₇ are as defined above.

In an exemplary embodiment, the compound represented by the formula (45) is represented by any one of formulae (45-1) to (45-6) below.

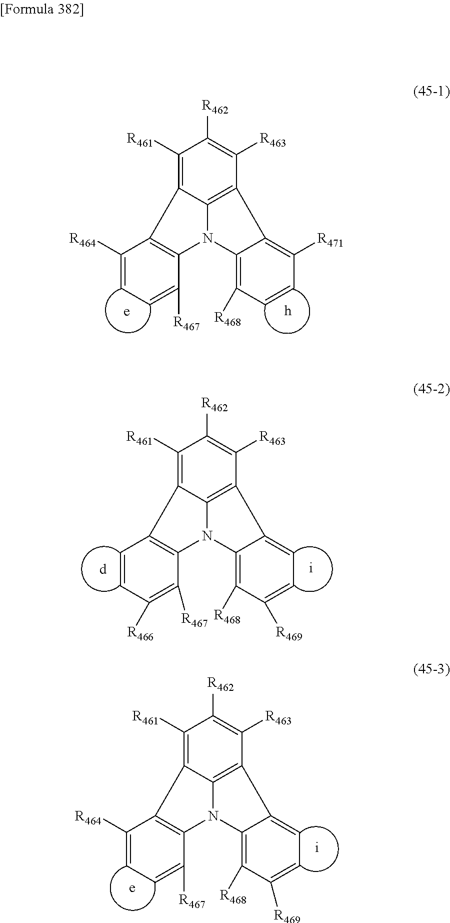

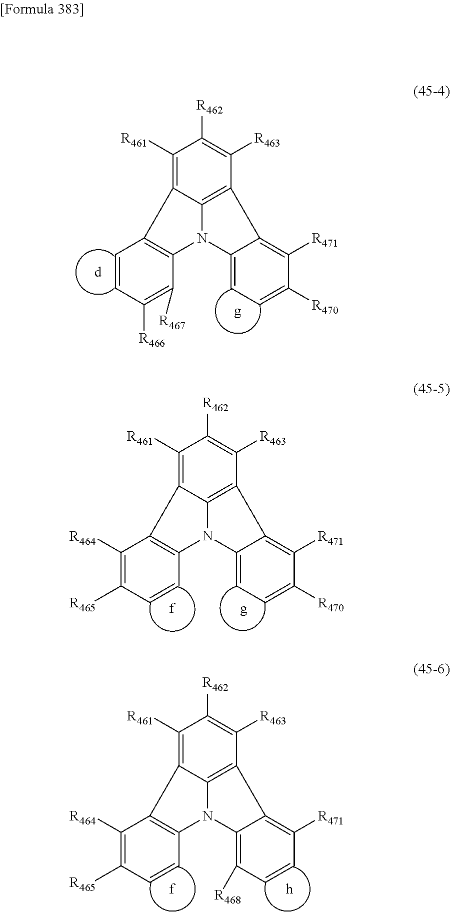

In the formulae (45-1) to (45-6):
rings d to i are each independently a substituted or unsubstituted monocyclic ring or a substituted or unsubstituted fused ring; and
R₄₆₁ to R₄₇₁ each independently represent the same as R₄₆₁ to R₄₇₁ in the formula (45).

In an exemplary embodiment, the compound represented by the formula (45) is represented by any one of formulae (45-7) to (45-12) below.

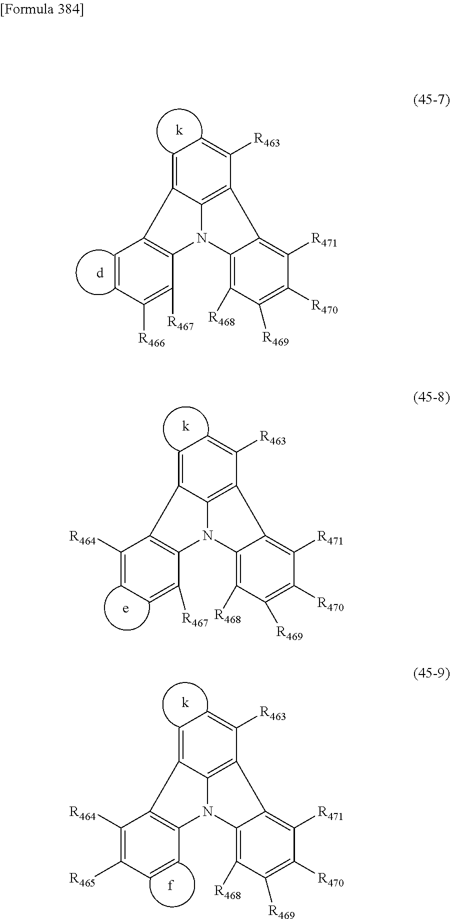

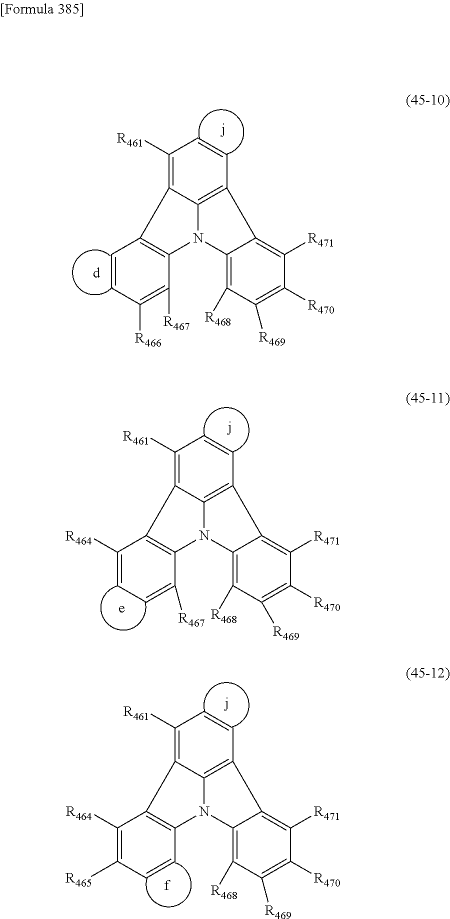

In the formulae (45-7) to (45-12):
rings d to f, k, and j are each independently a substituted or unsubstituted monocyclic ring or a substituted or unsubstituted fused ring; and
R₄₆₁ to R₄₇₁ each independently represent the same as R₄₆₁ to R₄₇₁ in the formula (45).

In an exemplary embodiment, the compound represented by the formula (45) is represented by any one of formulae (45-13) to (45-21) below.

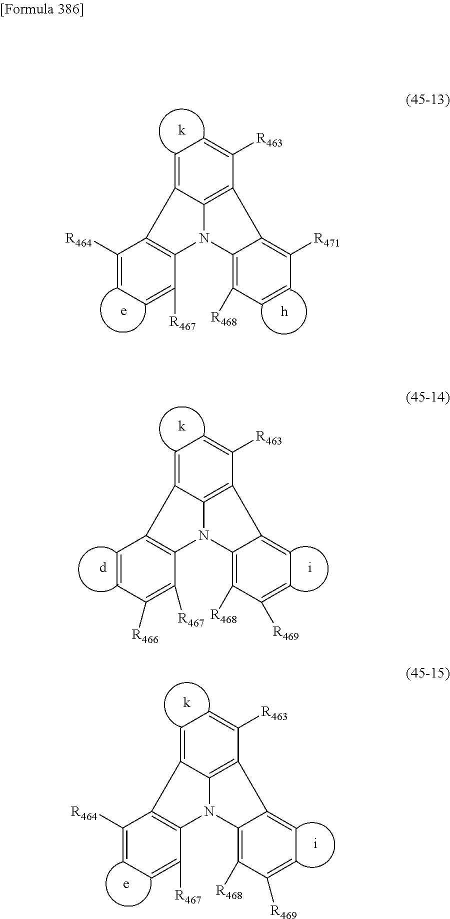

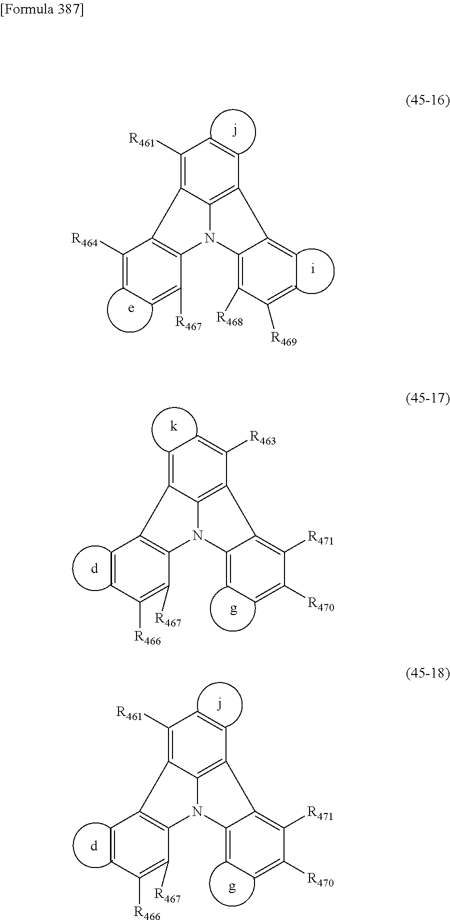

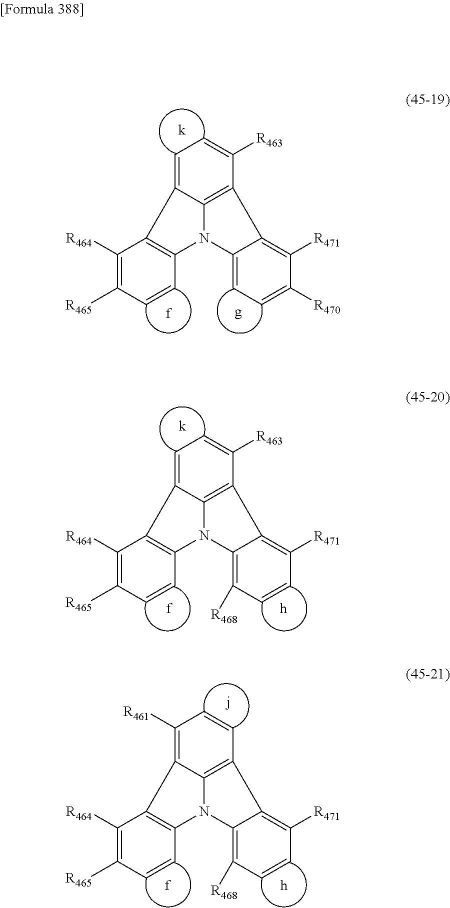

In the formulae (45-13) to (45-21):
rings d to k are each independently a substituted or unsubstituted monocyclic ring or a substituted or unsubstituted fused ring; and
R₄₆₁ to R₄₇₁ each independently represent the same as R₄₆₁ to R₄₇₁ in the formula (45).

When the ring g or the ring h further has a substituent, examples of the substituent include a substituted or unsubstituted alkyl group having 1 to 50 carbon atoms, a substituted or unsubstituted aryl group having 6 to 50 ring carbon atoms, a group represented by the formula (461), a group represented by the formula (463), or a group represented by the formula (464).

In an exemplary embodiment, the compound represented by the formula (45) is represented by any one of formulae (45-22) to (45-25) below.

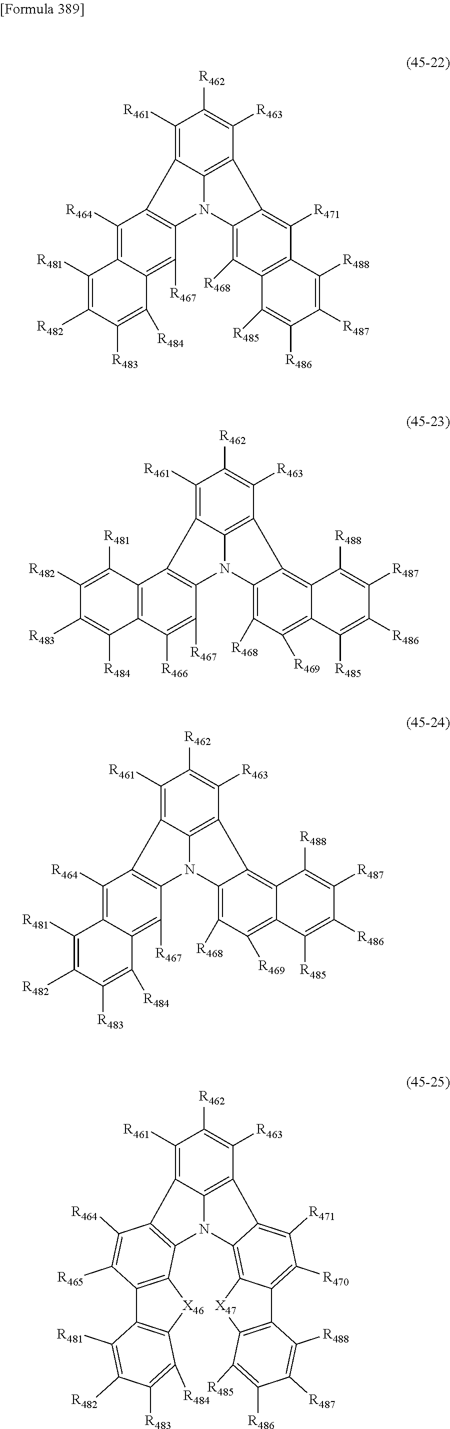

In the formulae (45-22) to (45-25):
X₄₆ and X₄₇ are each independently C(R₈₀₁)(R₈₀₂), NR₈₀₃, an oxygen atom, or a sulfur atom;
R₄₆₁ to R₄₇₁ and R₄₈₁ to R₄₈₈ each independently represent the same as R₄₆₁ to R₄₇₁ in the formula (45);
R₈₀₁, R₈₀₂ and R₈₀₃ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms; a substituted or unsubstituted alkyl group having 1 to 50 carbon atoms, or a substituted or unsubstituted aryl group having 6 to 50 ring carbon atoms;
when a plurality of R₈₀₁ are present, the plurality of R₈₀₁ are mutually the same or different;
when a plurality of R₈₀₂ are present, the plurality of R₈₀₂ are mutually the same or different; and
when a plurality of R₈₀₃ are present, the plurality of R₈₀₃ are mutually the same or different.

In an exemplary embodiment, the compound represented by the formula (45) is represented by a formula (45-26) below.

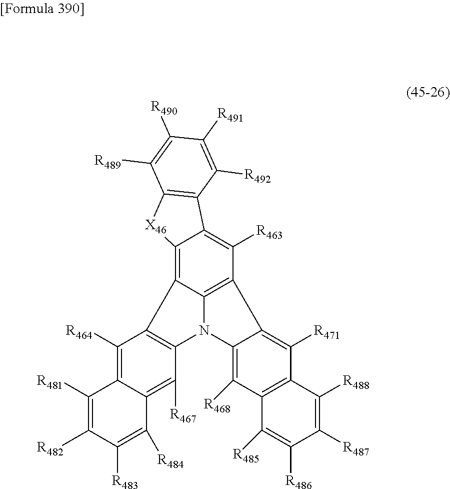

In the formula (45-26):
X₄₆ is C(R₈₀₁)(R₈₀₂), NR₈₀₃, an oxygen atom, or a sulfur atom;
R₄₆₃, R₄₆₄, R₄₆₇, R₄₆₈, R₄₇₁, and R₄₈₁ to R₄₉₂ each independently represent the same as R₄₆₁ to R₄₇₁ in the formula (45);
R₈₀₁, R₈₀₂ and R₈₀₃ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms; a substituted or unsubstituted alkyl group having 1 to 50 carbon atoms, or a substituted or unsubstituted aryl group having 6 to 50 ring carbon atoms;
when a plurality of R₈₀₁ are present, the plurality of R₈₀₁ are mutually the same or different;
when a plurality of R₈₀₂ are present, the plurality of R₈₀₂ are mutually the same or different; and
when a plurality of R₈₀₃ are present, the plurality of R₈₀₃ are mutually the same or different.

### Specific Examples of Compound Represented by Formula (4)

Specific examples of the compound represented by the formula (4) include compounds as below. In the specific examples below, Ph represents a phenyl group, and D represents a deuterium atom.

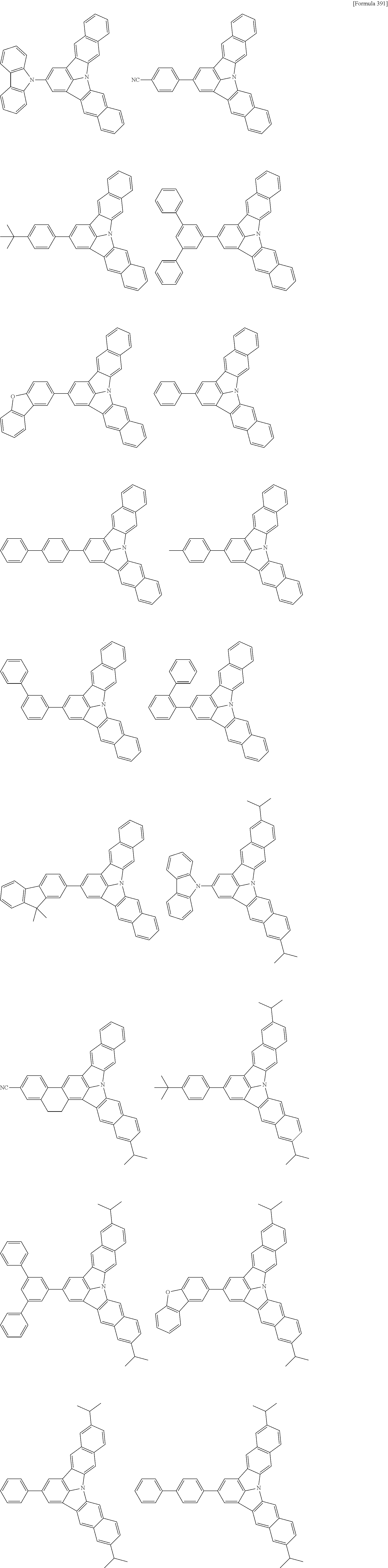

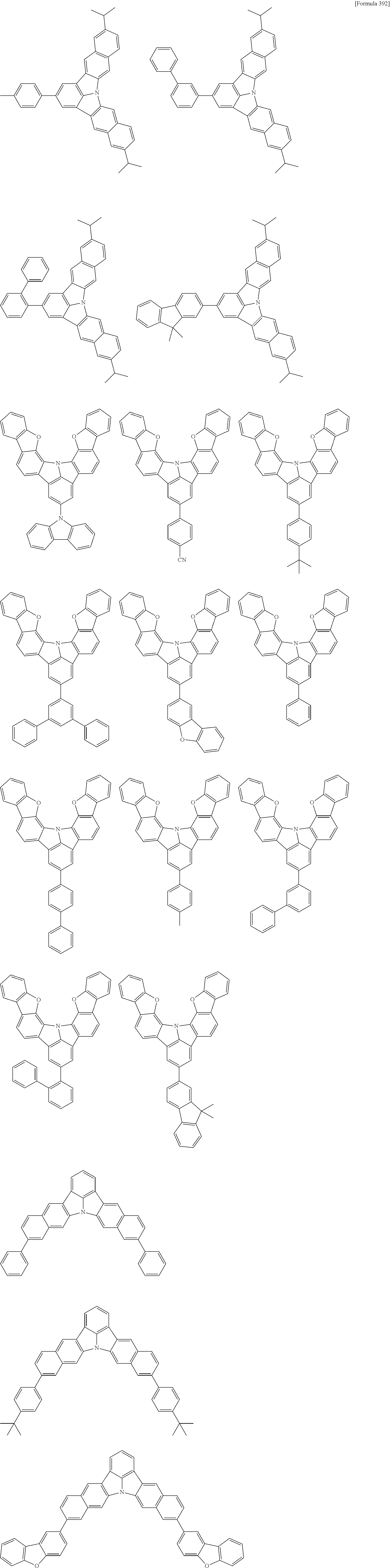

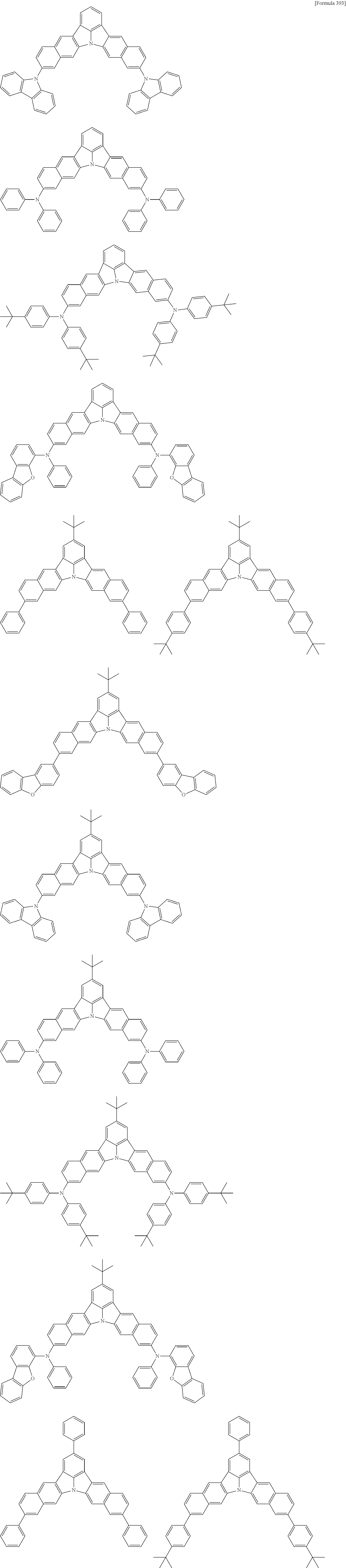

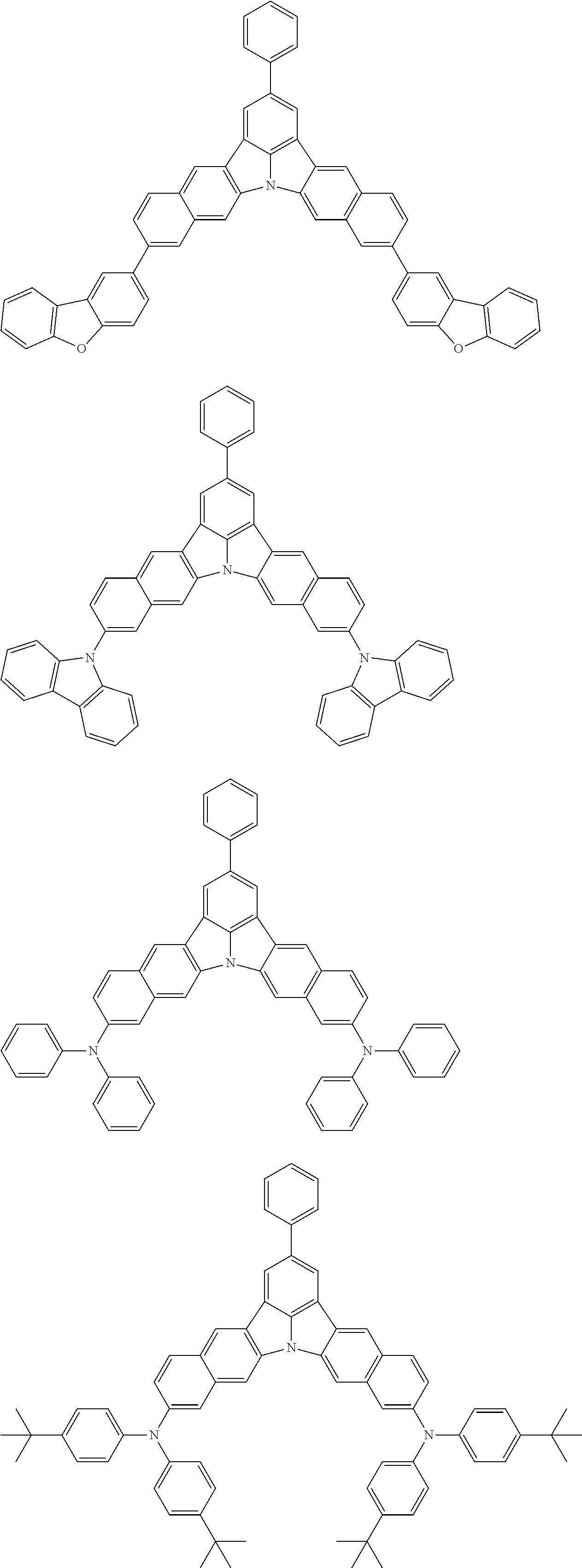 [SMILES: c1ccc(-c2cc3c4cc5ccc(-c6ccc7oc8ccccc8c7c6)cc5cc4n4c5cc6cc(-c7ccc8oc9ccccc9c8c7)ccc6cc5c(c2)c34)cc1 ; c1ccc(-c2cc3c4cc5ccc(-n6c7ccccc7c7ccccc76)cc5cc4n4c5cc6cc(-n7c8ccccc8c8ccccc87)ccc6cc5c(c2)c34)cc1 ; c1ccc(-c2cc3c4cc5ccc(N(c6ccccc6)c6ccccc6)cc5cc4n4c5cc6cc(N(c7ccccc7)c7ccccc7)ccc6cc5c(c2)c34)cc1 ; CC(C)(C)c1ccc(N(c2ccc(C(C)(C)C)cc2)c2ccc3cc4c5cc(-c6ccccc6)cc6c7cc8ccc(N(c9ccc(C(C)(C)C)cc9)c9ccc(C(C)(C)C)cc9)cc8cc7n(c4cc3c2)c56)cc1]

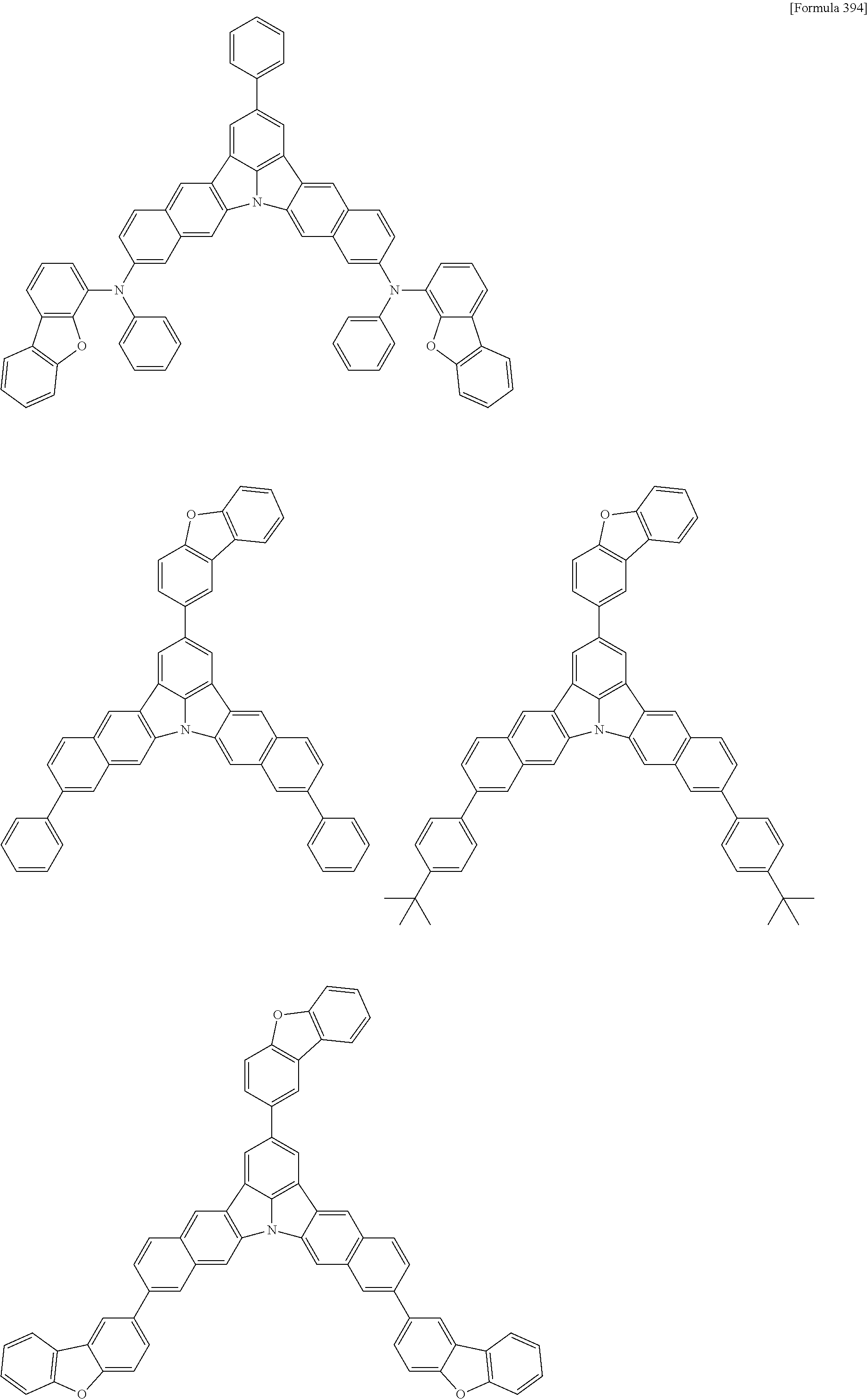

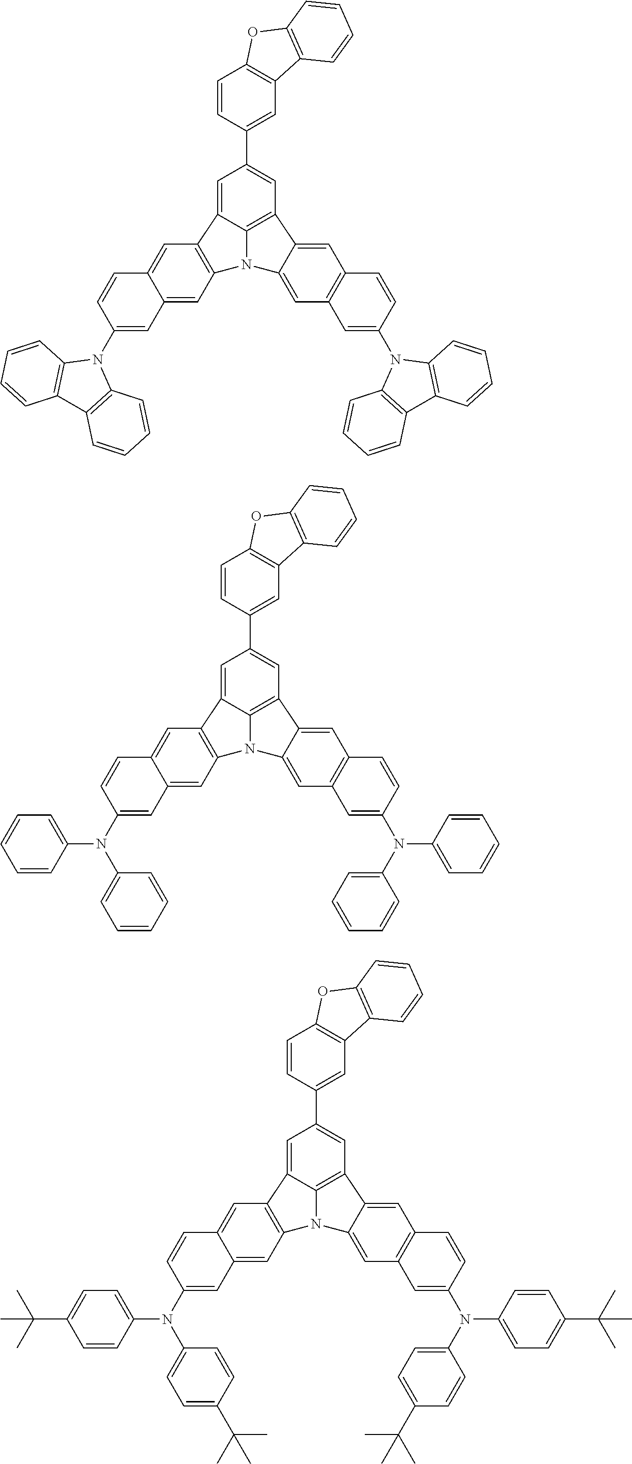

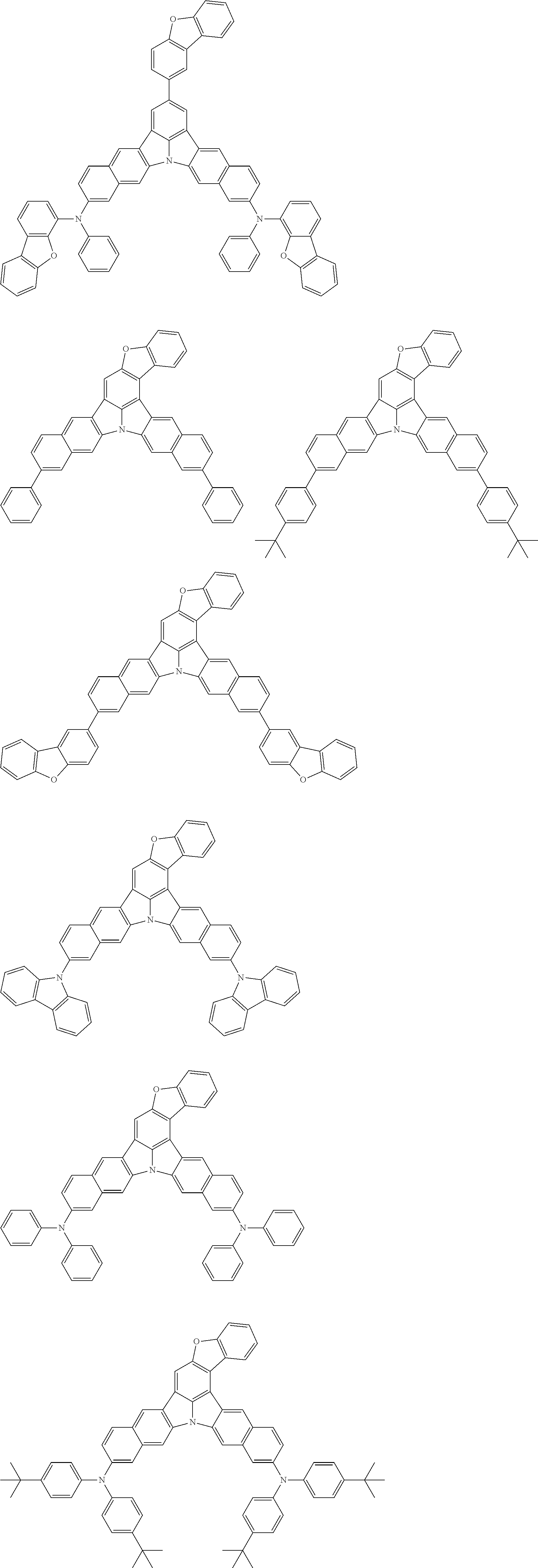

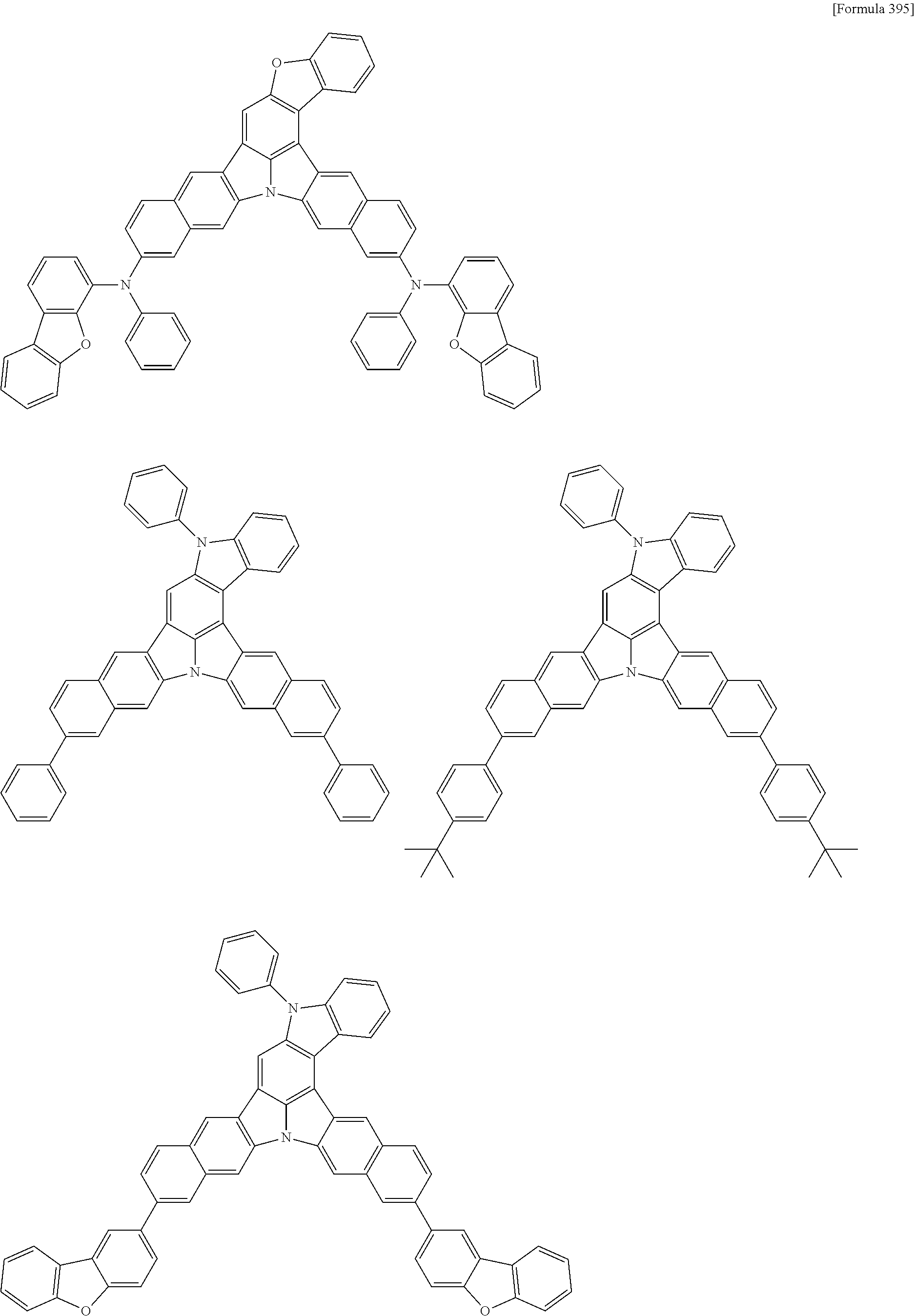

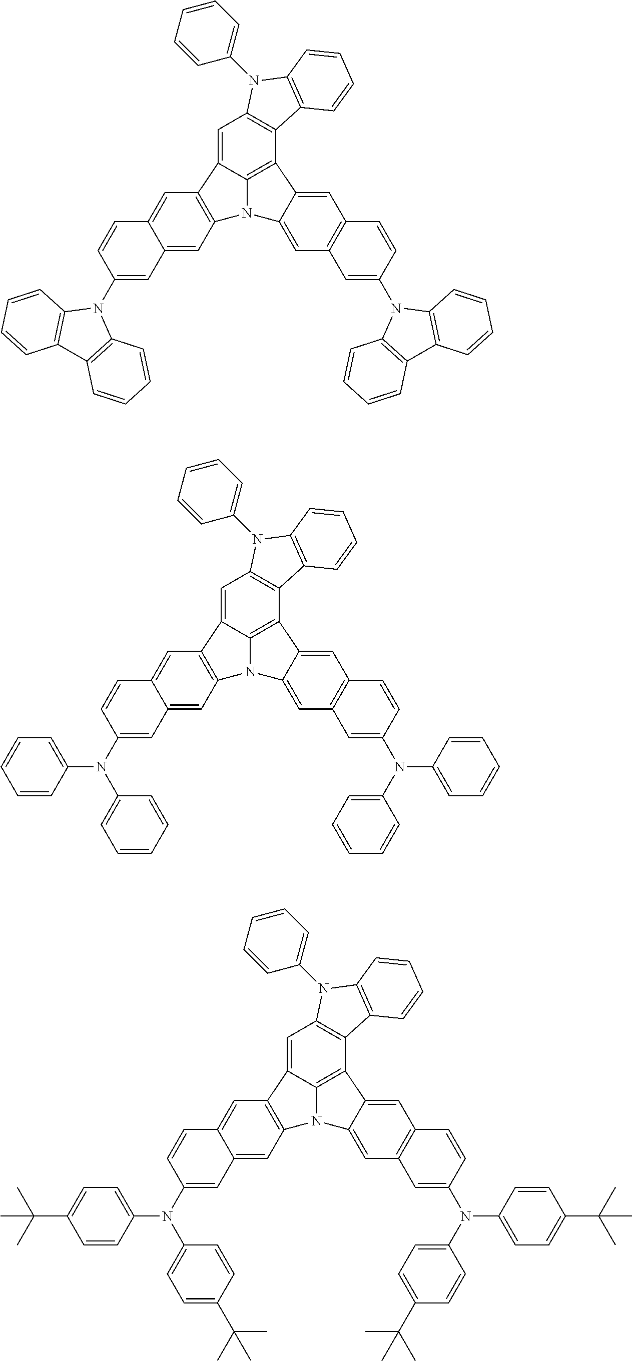 [SMILES: c1ccc(-n2c3ccccc3c3c4c5cc6ccc(-n7c8ccccc8c8ccccc87)cc6cc5n5c6cc7cc(-n8c9ccccc9c9ccccc98)ccc7cc6c(cc32)c45)cc1 ; c1ccc(N(c2ccccc2)c2ccc3cc4c5cc6c(c7ccccc7n6-c6ccccc6)c6c7cc8ccc(N(c9ccccc9)c9ccccc9)cc8cc7n(c4cc3c2)c56)cc1 ; CC(C)(C)c1ccc(N(c2ccc(C(C)(C)C)cc2)c2ccc3cc4c5cc6c(c7ccccc7n6-c6ccccc6)c6c7cc8ccc(N(c9ccc(C(C)(C)C)cc9)c9ccc(C(C)(C)C)cc9)cc8cc7n(c4cc3c2)c56)cc1]

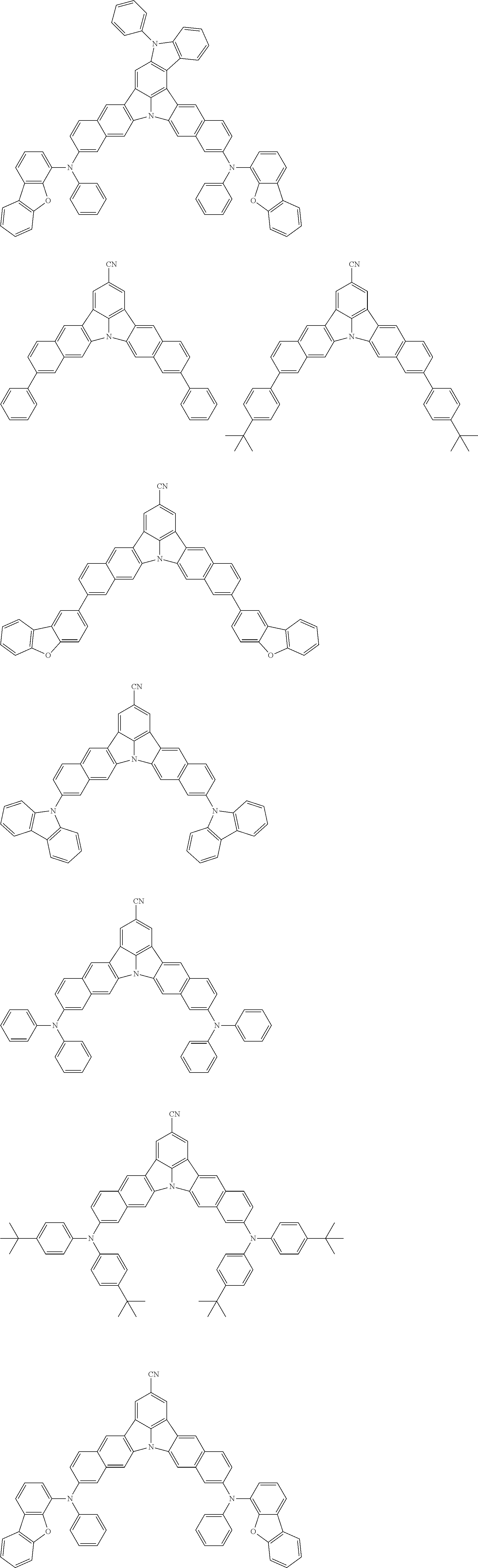

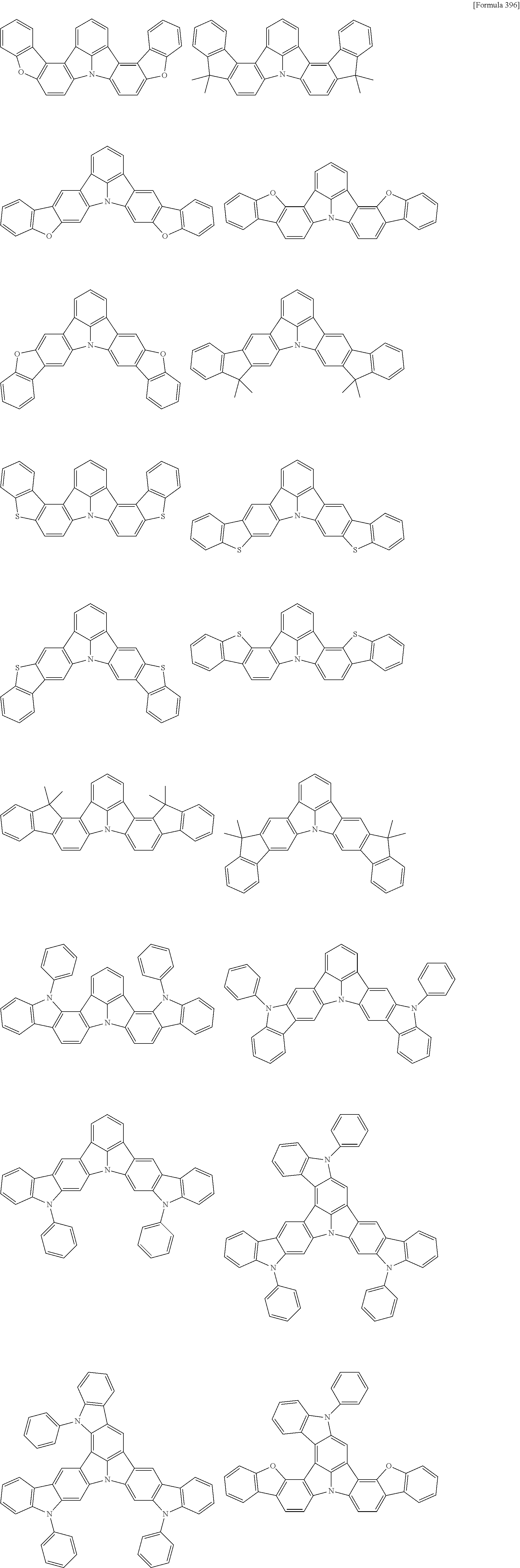

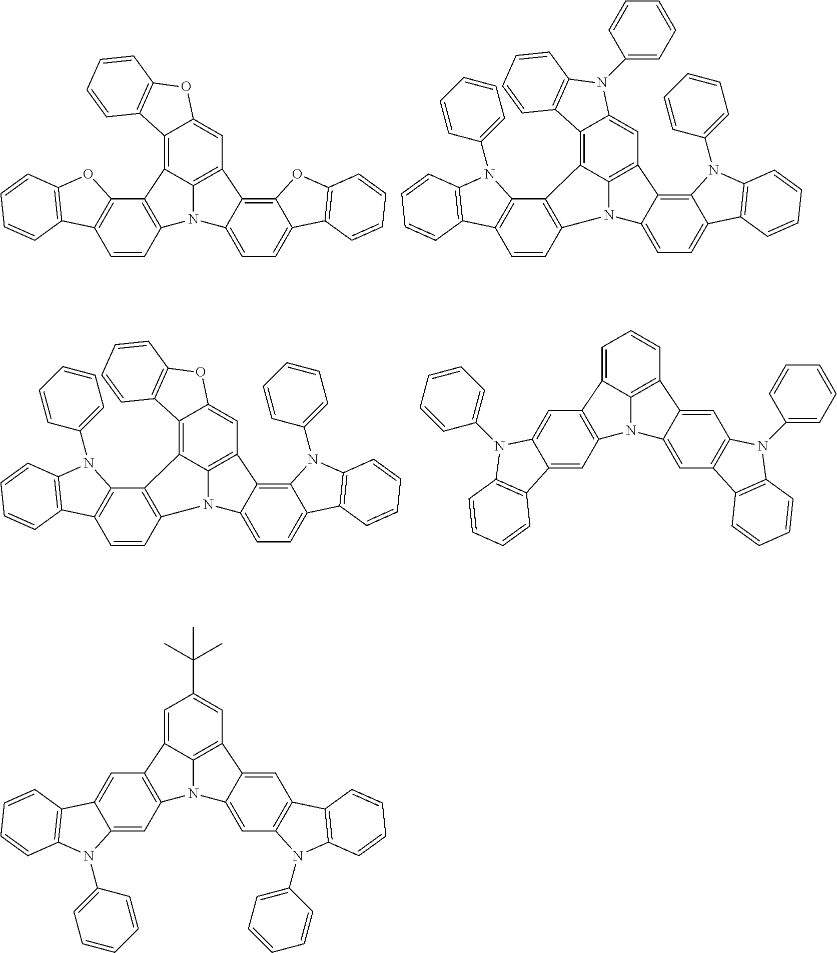

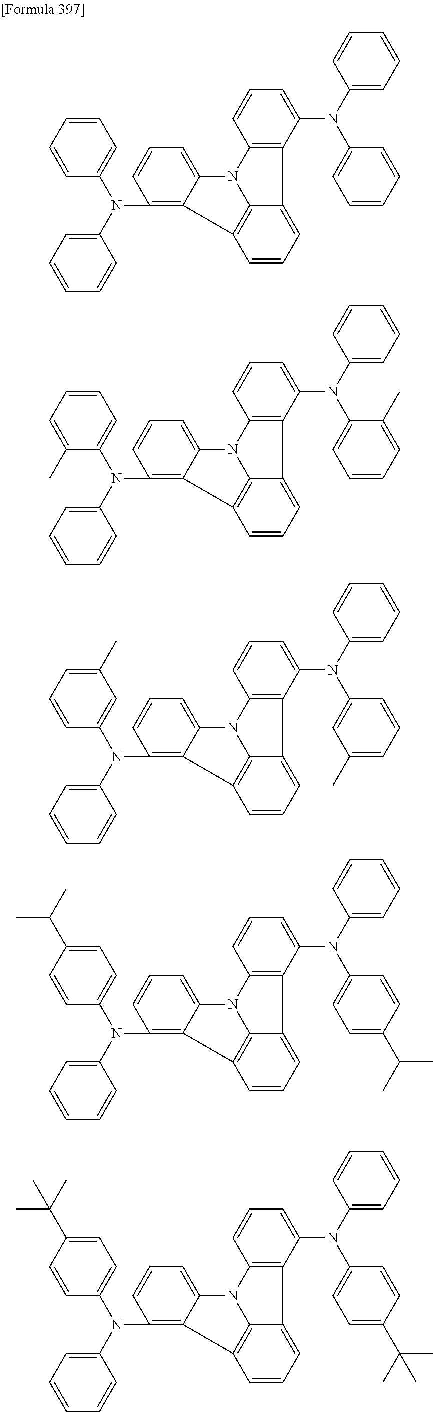

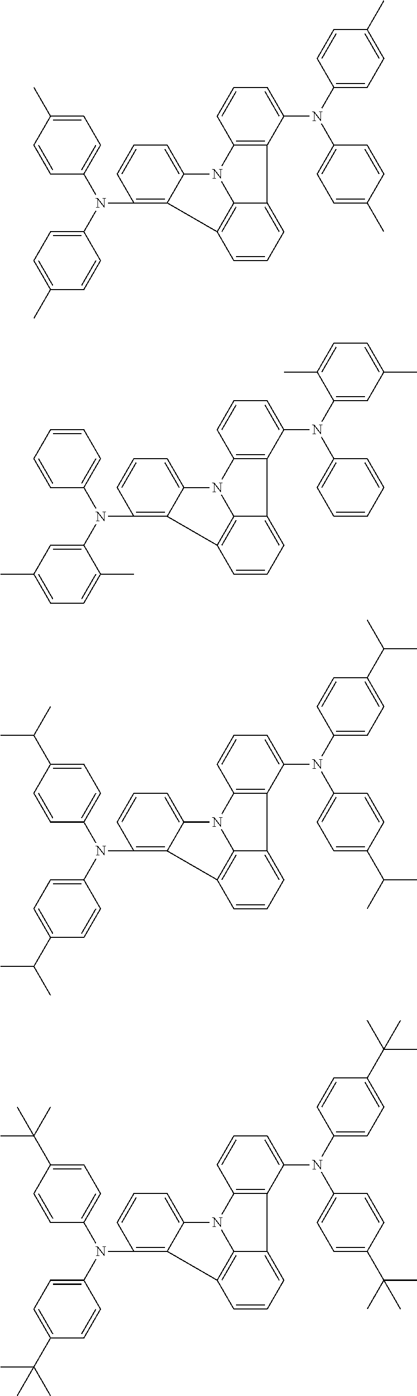

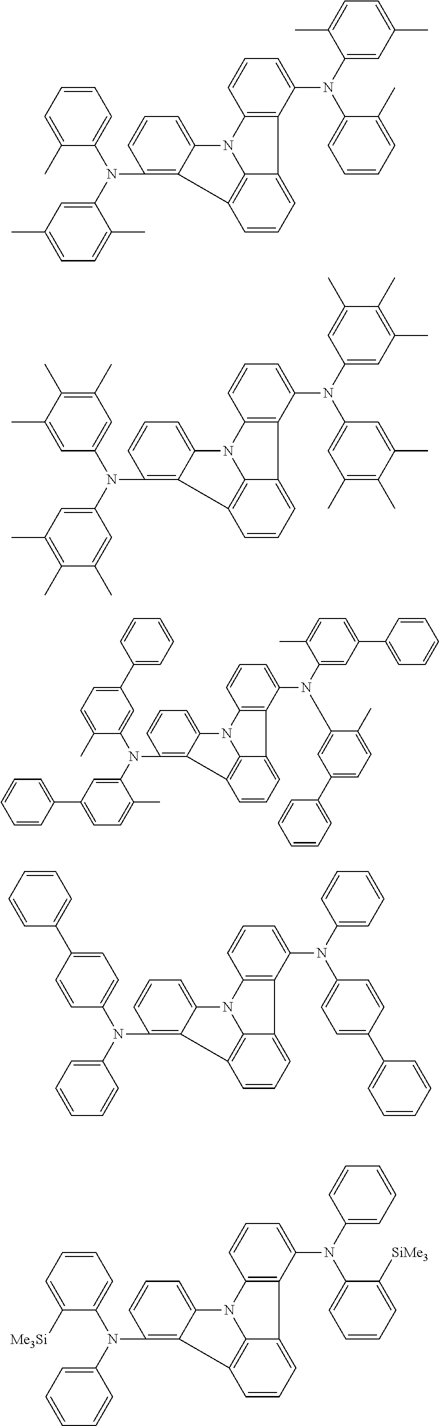

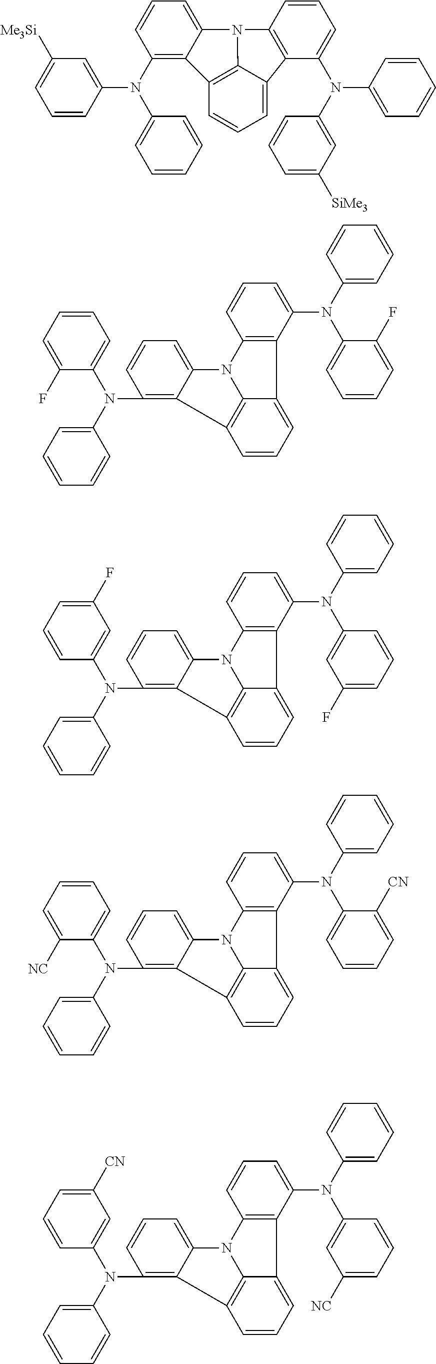

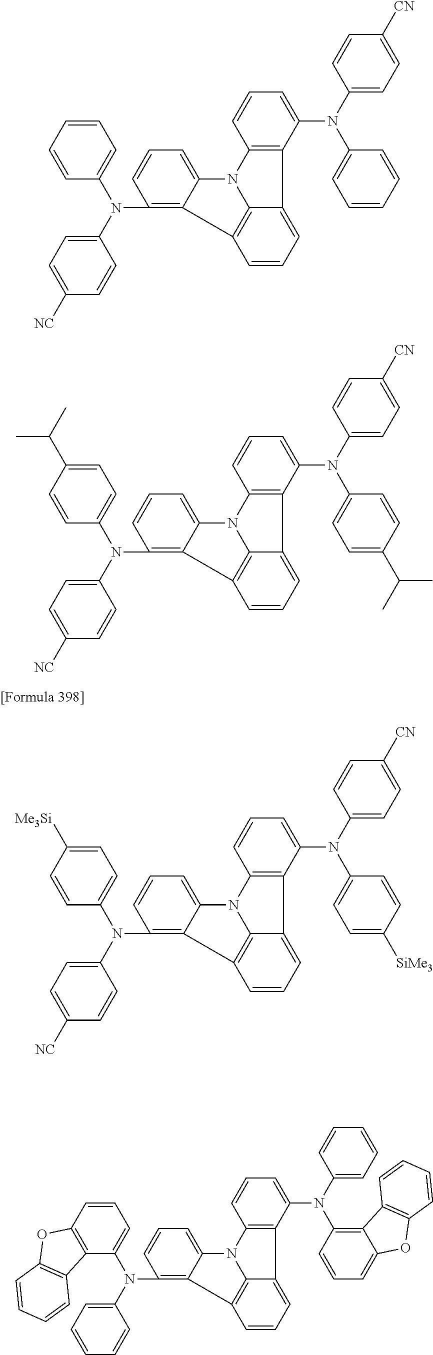 [SMILES: N#Cc1ccc(N(c2ccccc2)c2cccc3c2c2cccc4c5c(N(c6ccccc6)c6ccc(C#N)cc6)cccc5n3c24)cc1 ; CC(C)c1ccc(N(c2ccc(C#N)cc2)c2cccc3c2c2cccc4c5c(N(c6ccc(C#N)cc6)c6ccc(C(C)C)cc6)cccc5n3c24)cc1 ; C[Si](C)(C)c1ccc(N(c2ccc(C#N)cc2)c2cccc3c2c2cccc4c5c(N(c6ccc(C#N)cc6)c6ccc([Si](C)(C)C)cc6)cccc5n3c24)cc1 ; c1ccc(N(c2cccc3oc4ccccc4c23)c2cccc3c2c2cccc4c5c(N(c6ccccc6)c6cccc7oc8ccccc8c67)cccc5n3c24)cc1]

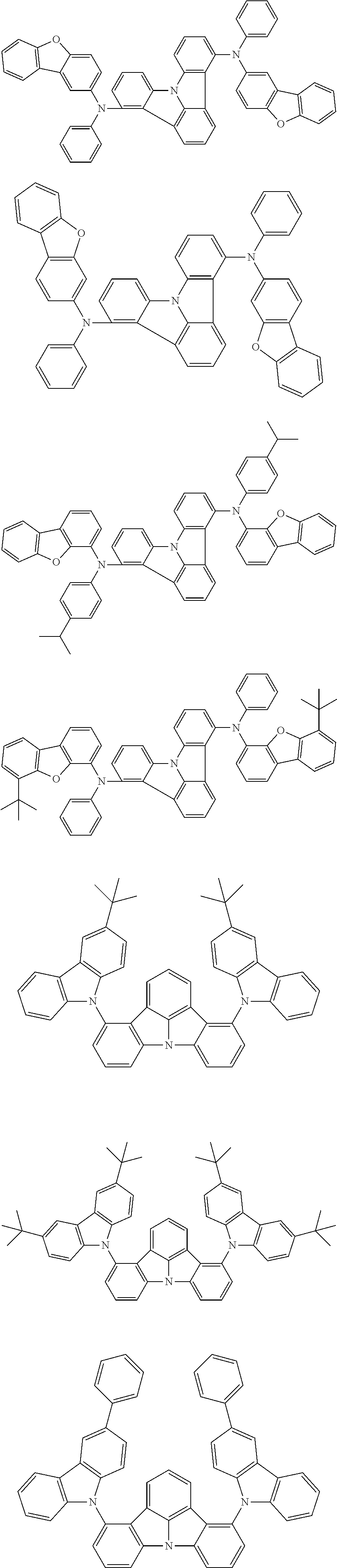

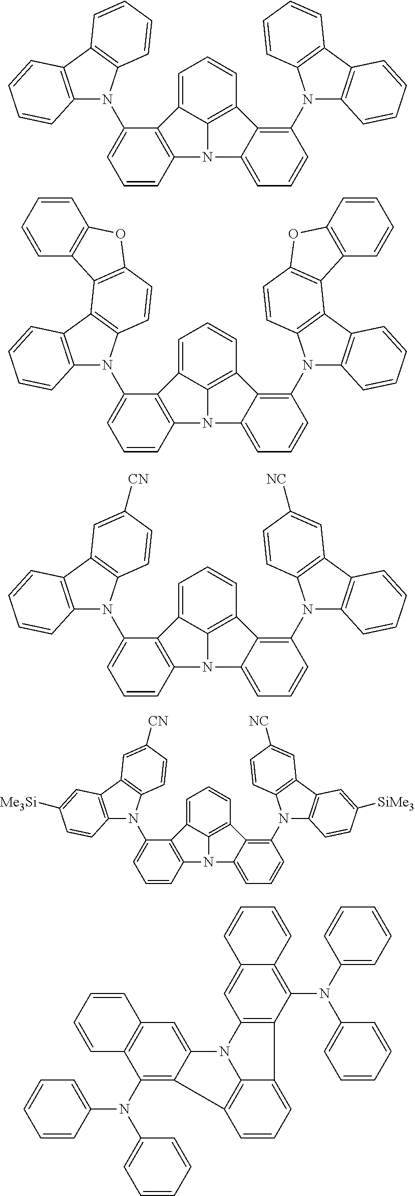

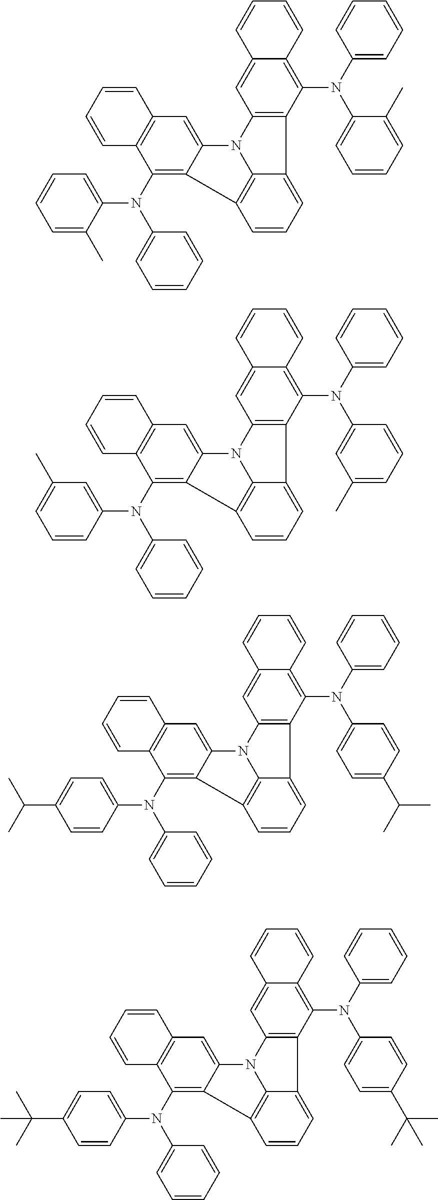

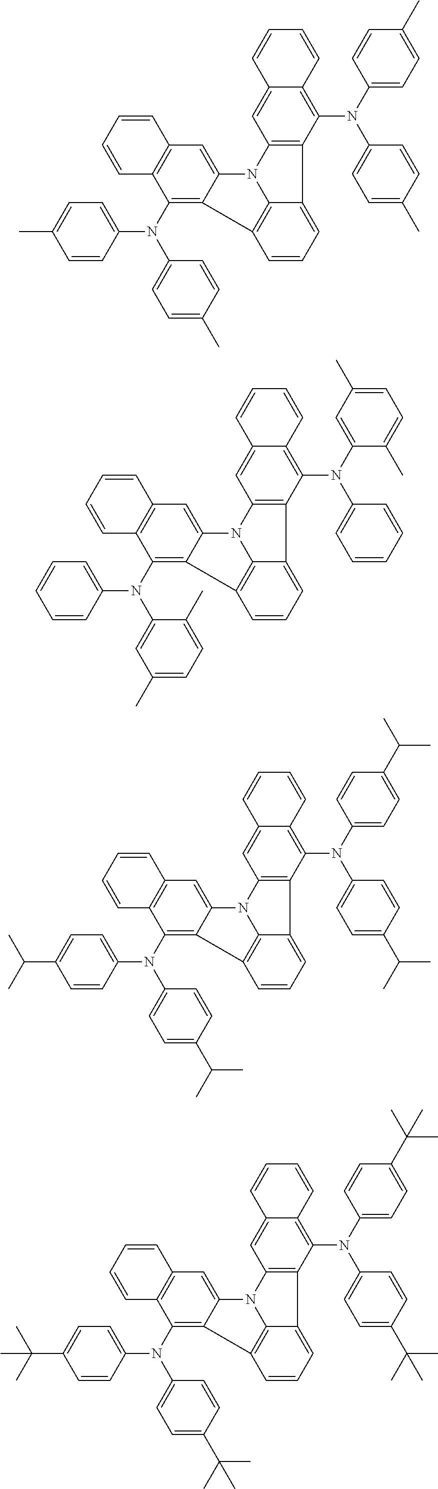

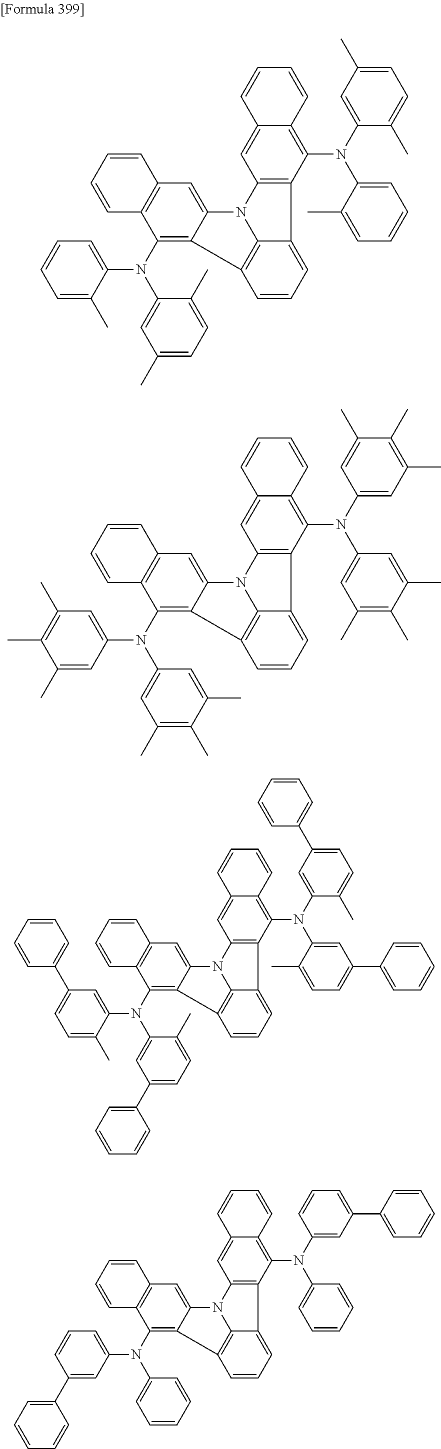

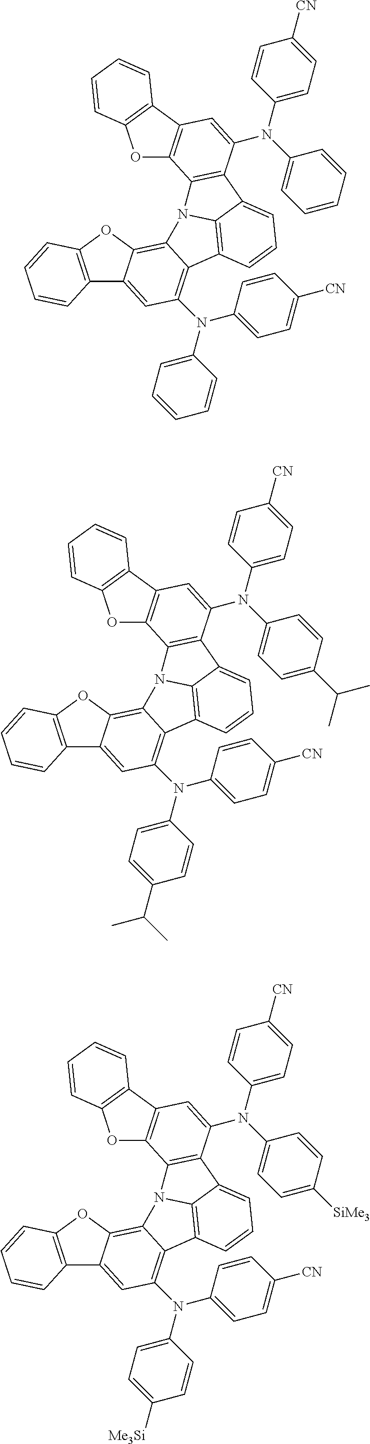 [SMILES: N#Cc1ccc(N(c2ccccc2)c2cc3c4ccccc4oc3c3c2c2cccc4c5c(N(c6ccccc6)c6ccc(C#N)cc6)cc6c7ccccc7oc6c5n3c24)cc1 ; CC(C)c1ccc(N(c2ccc(C#N)cc2)c2cc3c4ccccc4oc3c3c2c2cccc4c5c(N(c6ccc(C#N)cc6)c6ccc(C(C)C)cc6)cc6c7ccccc7oc6c5n3c24)cc1 ; C[Si](C)(C)c1ccc(N(c2ccc(C#N)cc2)c2cc3c4ccccc4oc3c3c2c2cccc4c5c(N(c6ccc(C#N)cc6)c6ccc([Si](C)(C)C)cc6)cc6c7ccccc7oc6c5n3c24)cc1]

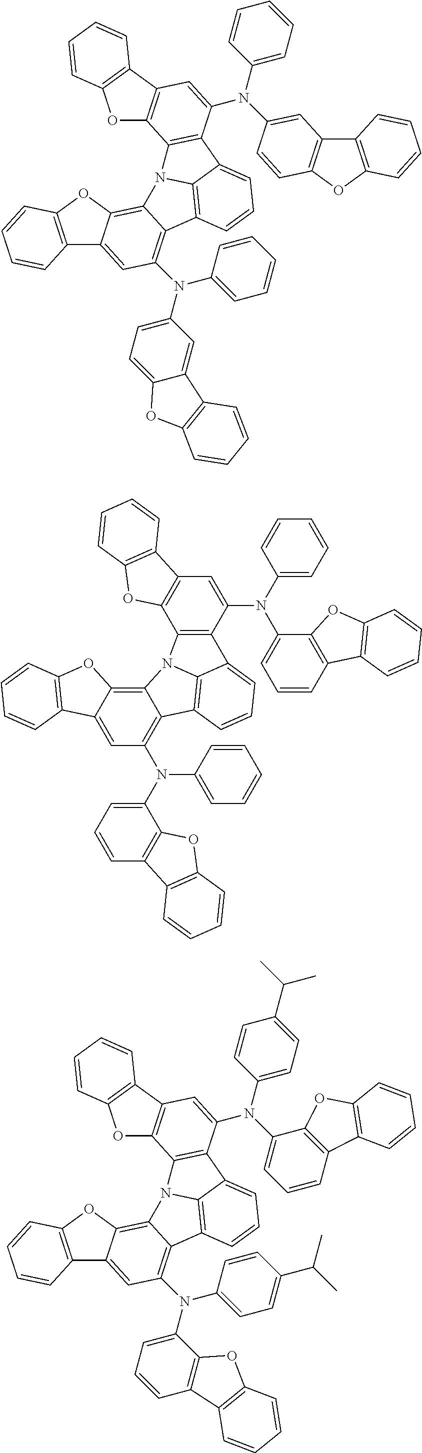 [SMILES: c1ccc(N(c2ccc3oc4ccccc4c3c2)c2cc3c4ccccc4oc3c3c2c2cccc4c5c(N(c6ccccc6)c6ccc7oc8ccccc8c7c6)cc6c7ccccc7oc6c5n3c24)cc1 ; c1ccc(N(c2cccc3c2oc2ccccc23)c2cc3c4ccccc4oc3c3c2c2cccc4c5c(N(c6ccccc6)c6cccc7c6oc6ccccc67)cc6c7ccccc7oc6c5n3c24)cc1 ; CC(C)c1ccc(N(c2cccc3c2oc2ccccc23)c2cc3c4ccccc4oc3c3c2c2cccc4c5c(N(c6ccc(C(C)C)cc6)c6cccc7c6oc6ccccc67)cc6c7ccccc7oc6c5n3c24)cc1]

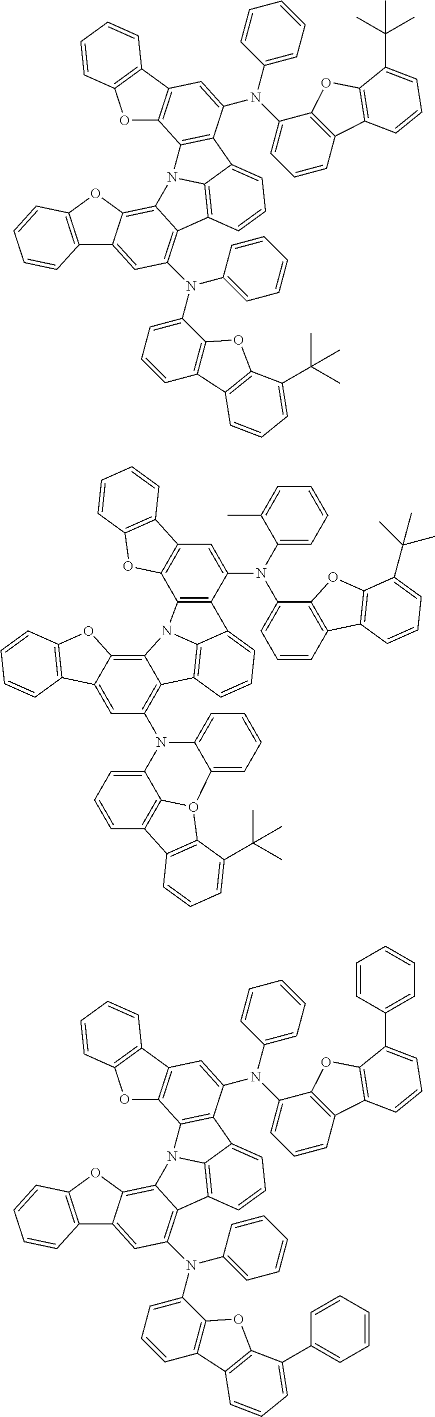

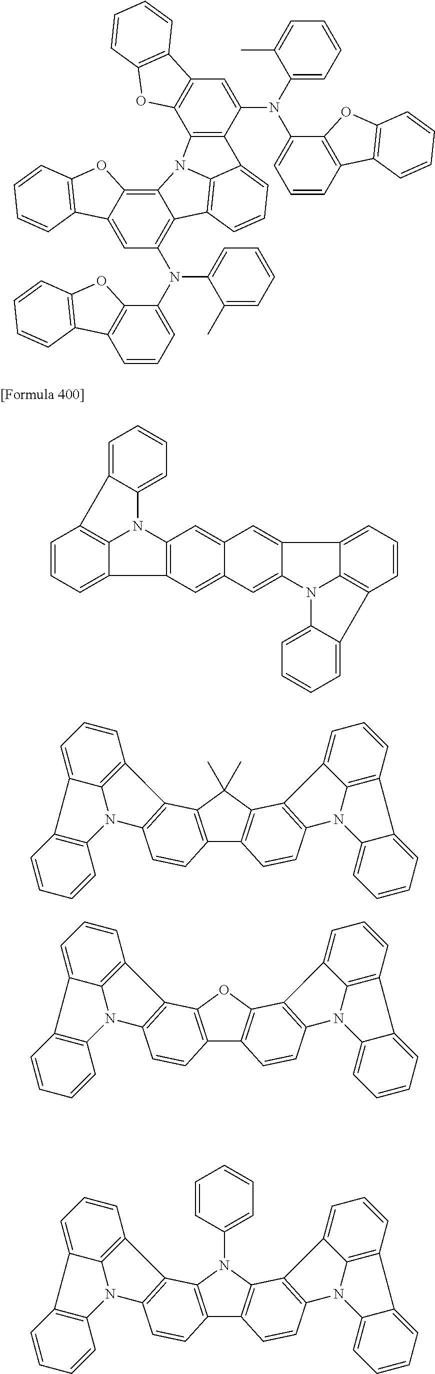

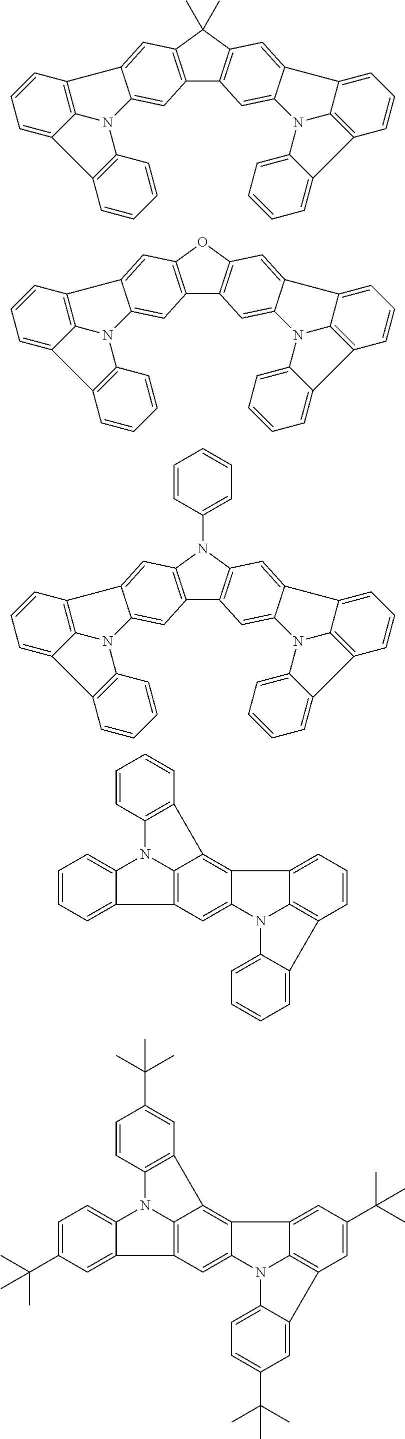

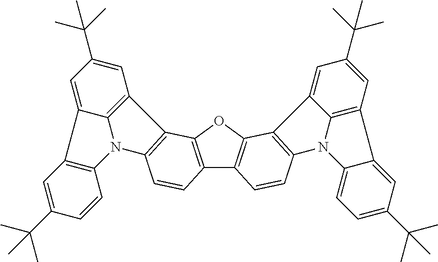 [SMILES: CC(C)(C)c1ccc2c(c1)c1cc(C(C)(C)C)cc3c4c5oc6c(ccc7c6c6cc(C(C)(C)C)cc8c9cc(C(C)(C)C)ccc9n7c86)c5ccc4n2c13]

### Compound Represented by Formula (5)

The compound represented by the formula (5) will be described below. The compound represented by the formula (5) corresponds to a compound represented by the formula (41-3) described above.

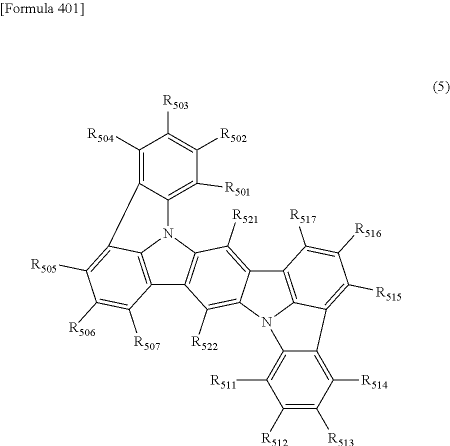

In the formula (5):
at least one combination of adjacent two or more of R₅₀₁ to R₅₀₇ and R₅₁₁ to R₅₁₇ are mutually bonded to form a substituted or unsubstituted monocyclic ring, mutually bonded to form a substituted or unsubstituted fused ring, or not mutually bonded; and
R₅₀₁ to R₅₀₇ and R₅₁₁ to R₅₁₇ forming neither the monocyclic ring nor the fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

R₅₂₁ and R₅₂₂ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

“A combination of adjacent two or more of R₅₀₁ to R₅₀₇ and R₅₁₁ to R₅₁₇” refers to, for instance, a combination of R₅₀₁ and R₅₀₂, a combination of R₅₀₂ and R₅₀₃, a combination of R₅₀₃ and R₅₀₄, a combination of R₅₀₅ and R₅₀₆, a combination of R₅₀₆ and R₅₀₇, and a combination of R₅₀₁, R₅₀₂, and R₅₀₃.

In an exemplary embodiment, at least one, preferably two of R₅₀₁ to R₅₀₇ or R₅₁₁ to R₅₁₇ are each a group represented by —N(R₉₀₆)(R₉₀₇).

In an exemplary embodiment, R₅₀₁ to R₅₀₇ and R₅₁₁ to R₅₁₇ are each independently a hydrogen atom, a substituted or unsubstituted aryl group having 6 to 50 ring carbon atoms, or a substituted or unsubstituted heterocyclic group having 5 to 50 ring atoms.

In an exemplary embodiment, the compound represented by the formula (5) is a compound represented by a formula (52) below.

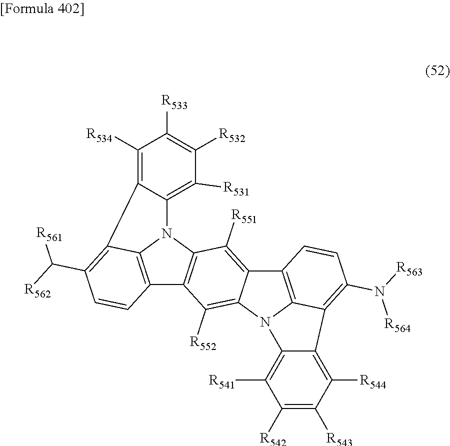

In the formula (52):
at least one combination of adjacent two or more of R₅₃₁ to R₅₃₄ and R₅₄₁ to R₅₄₄ are mutually bonded to form a substituted or unsubstituted monocyclic ring, mutually bonded to form a substituted or unsubstituted fused ring, or not mutually bonded;
R₅₃₁ to R₅₃₄ and R₅₄₁ to R₅₄₄ forming neither the monocyclic ring nor the fused ring, and R₅₅₁ and R₅₅₂ are each independently a hydrogen atom, a substituted or unsubstituted aryl group having 6 to 50 ring carbon atoms, or a substituted or unsubstituted heterocyclic group having 5 to 50 ring atoms; and
R₅₆₁ to R₅₆₄ are each independently a substituted or unsubstituted aryl group having 6 to 50 ring carbon atoms, or a substituted or unsubstituted heterocyclic group having 5 to 50 ring atoms.

In an exemplary embodiment, the compound represented by the formula (5) is a compound represented by a formula (53) below.

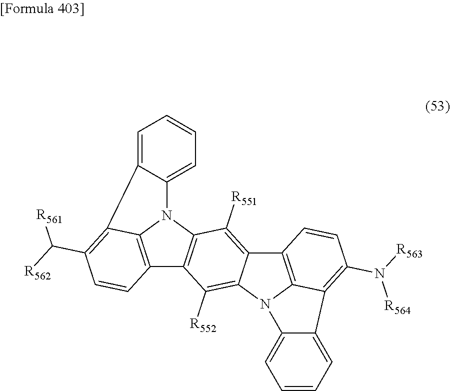

In the formula (53), R₅₅₁, R₅₅₂ and R₅₆₁ to R₅₆₄ each independently represent the same as R₅₅₁, R₅₅₂ and R₅₆₁ to R₅₆₄ in the formula (52).

In an exemplary embodiment, R₅₆₁ to R₅₆₄ in the formulae (52) and (53) are each independently a substituted or unsubstituted aryl group having 6 to 50 ring carbon atoms (preferably a phenyl group).

In an exemplary embodiment, R₅₂₁ and R₅₂₂ in the formula (5) and R₅₅₁ and R₅₅₂ in the formulae (52) and (53) are each a hydrogen atom.

In an exemplary embodiment, the substituent for the “substituted or unsubstituted” group in the formulae (5), (52) and (53) is a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

Specific Examples of Compound Represented by Formula (5) Specific examples of the compound represented by the formula (5) include compounds as below.

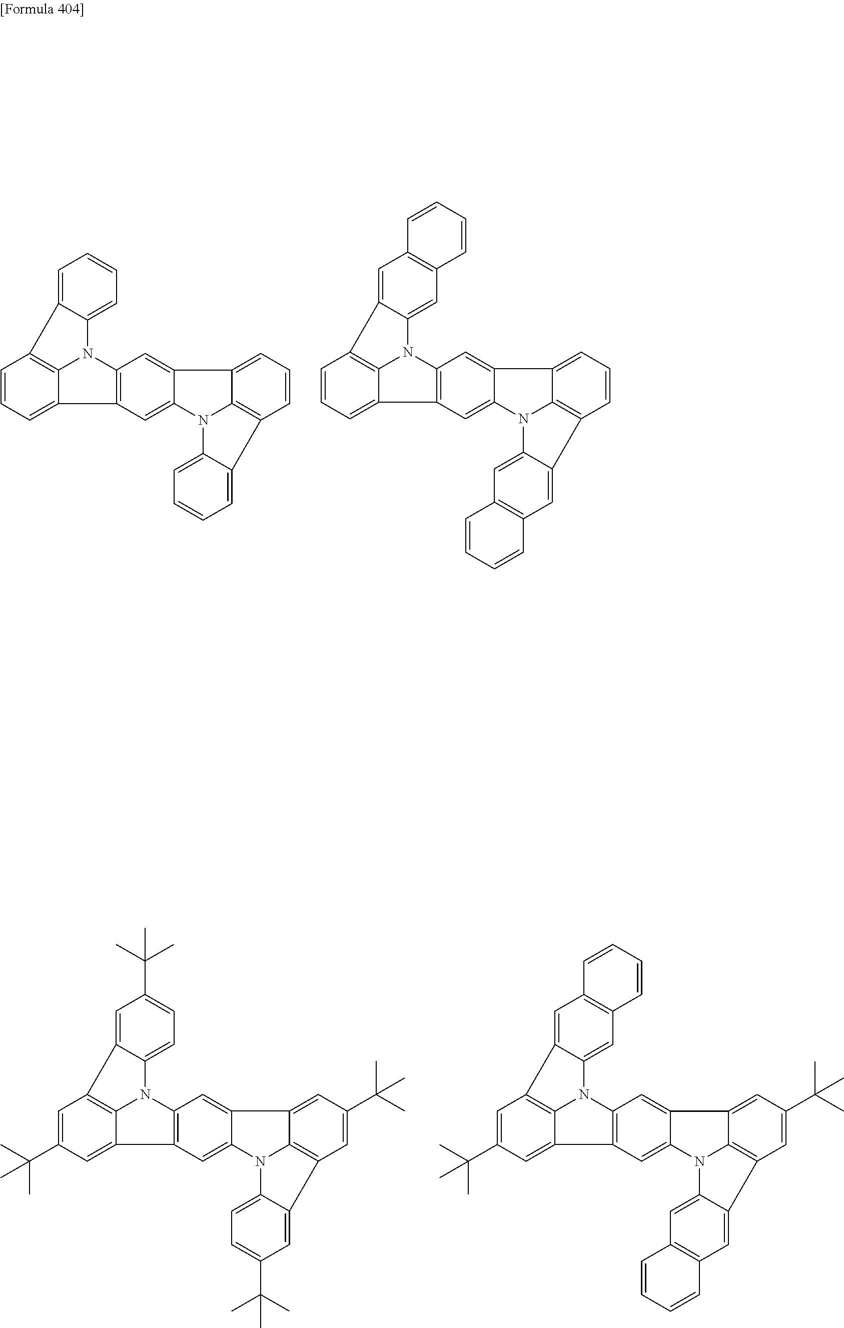

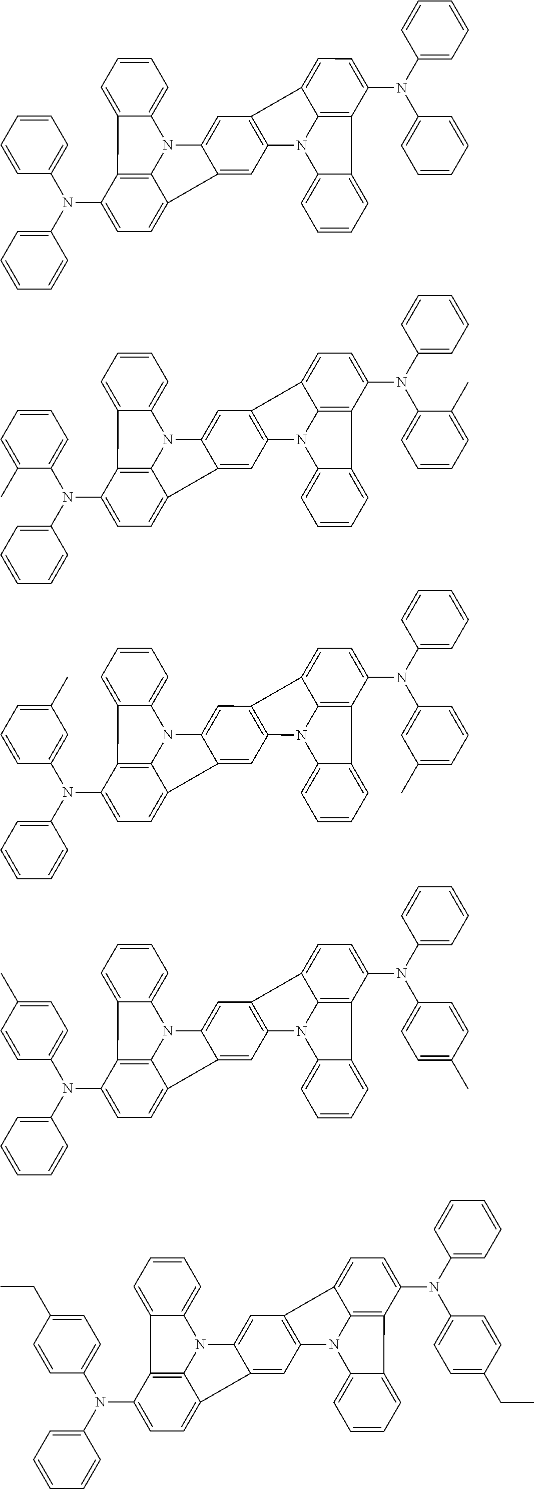 [SMILES: c1ccc(N(c2ccccc2)c2ccc3c4cc5c(cc4n4c6ccccc6c2c34)c2ccc(N(c3ccccc3)c3ccccc3)c3c4ccccc4n5c23)cc1 ; Cc1ccccc1N(c1ccccc1)c1ccc2c3cc4c(cc3n3c5ccccc5c1c23)c1ccc(N(c2ccccc2)c2ccccc2C)c2c3ccccc3n4c12 ; Cc1cccc(N(c2ccccc2)c2ccc3c4cc5c(cc4n4c6ccccc6c2c34)c2ccc(N(c3ccccc3)c3cccc(C)c3)c3c4ccccc4n5c23)c1 ; Cc1ccc(N(c2ccccc2)c2ccc3c4cc5c(cc4n4c6ccccc6c2c34)c2ccc(N(c3ccccc3)c3ccc(C)cc3)c3c4ccccc4n5c23)cc1 ; CCc1ccc(N(c2ccccc2)c2ccc3c4cc5c(cc4n4c6ccccc6c2c34)c2ccc(N(c3ccccc3)c3ccc(CC)cc3)c3c4ccccc4n5c23)cc1]

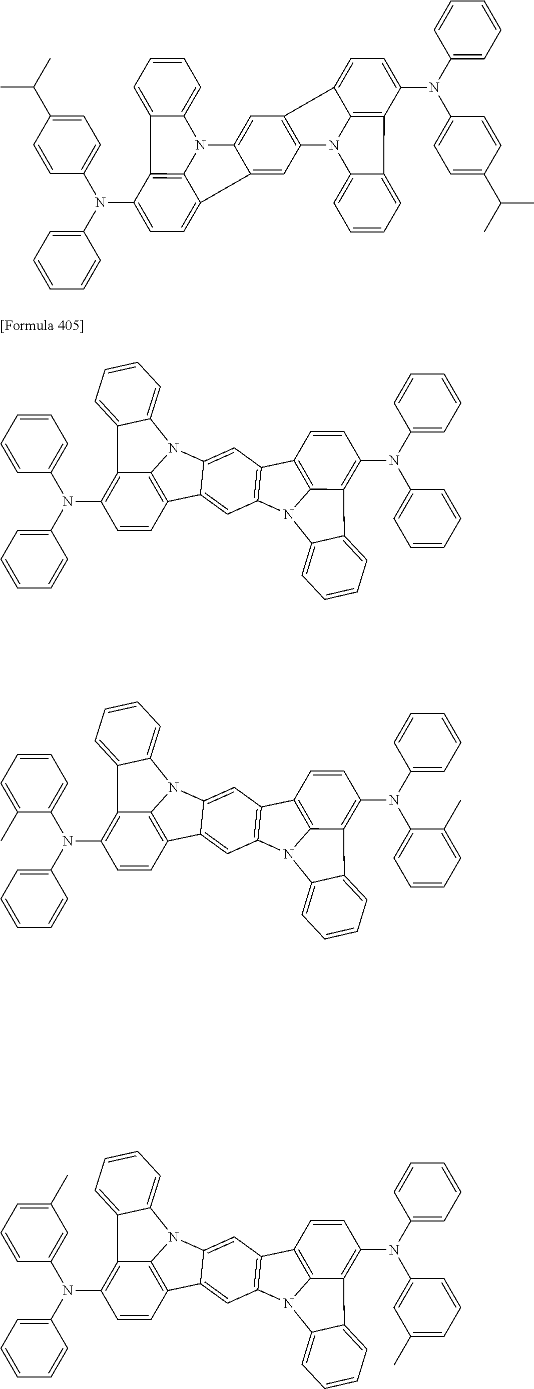 [SMILES: CC(C)c1ccc(N(c2ccccc2)c2ccc3c4cc5c(cc4n4c6ccccc6c2c34)c2ccc(N(c3ccccc3)c3ccc(C(C)C)cc3)c3c4ccccc4n5c23)cc1 ; c1ccc(N(c2ccccc2)c2ccc3c4cc5c(cc4n4c6ccccc6c2c34)c2ccc(N(c3ccccc3)c3ccccc3)c3c4ccccc4n5c23)cc1 ; Cc1ccccc1N(c1ccccc1)c1ccc2c3cc4c(cc3n3c5ccccc5c1c23)c1ccc(N(c2ccccc2)c2ccccc2C)c2c3ccccc3n4c12 ; Cc1cccc(N(c2ccccc2)c2ccc3c4cc5c(cc4n4c6ccccc6c2c34)c2ccc(N(c3ccccc3)c3cccc(C)c3)c3c4ccccc4n5c23)c1]

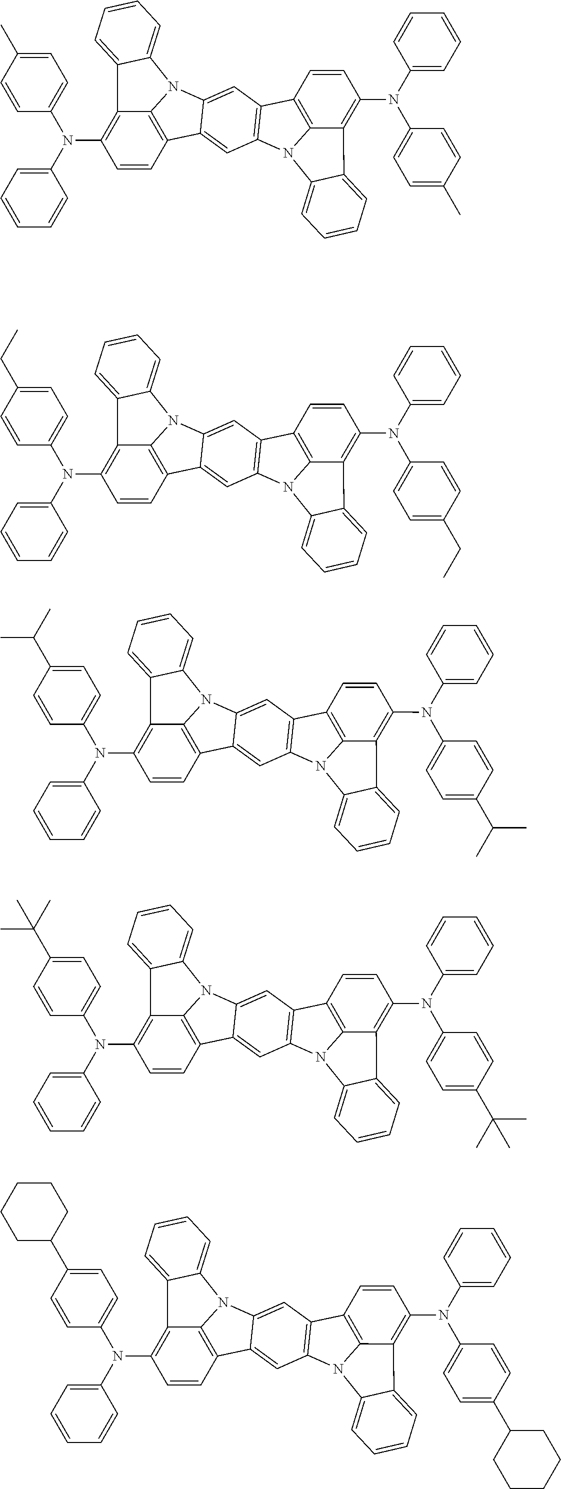

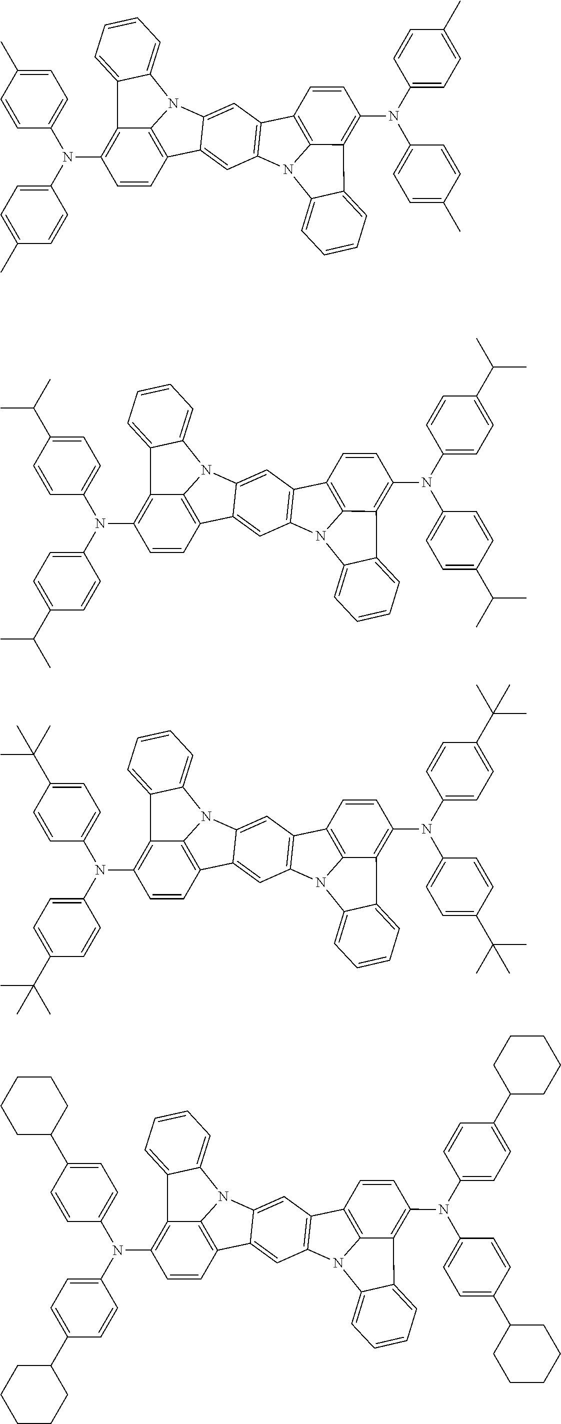

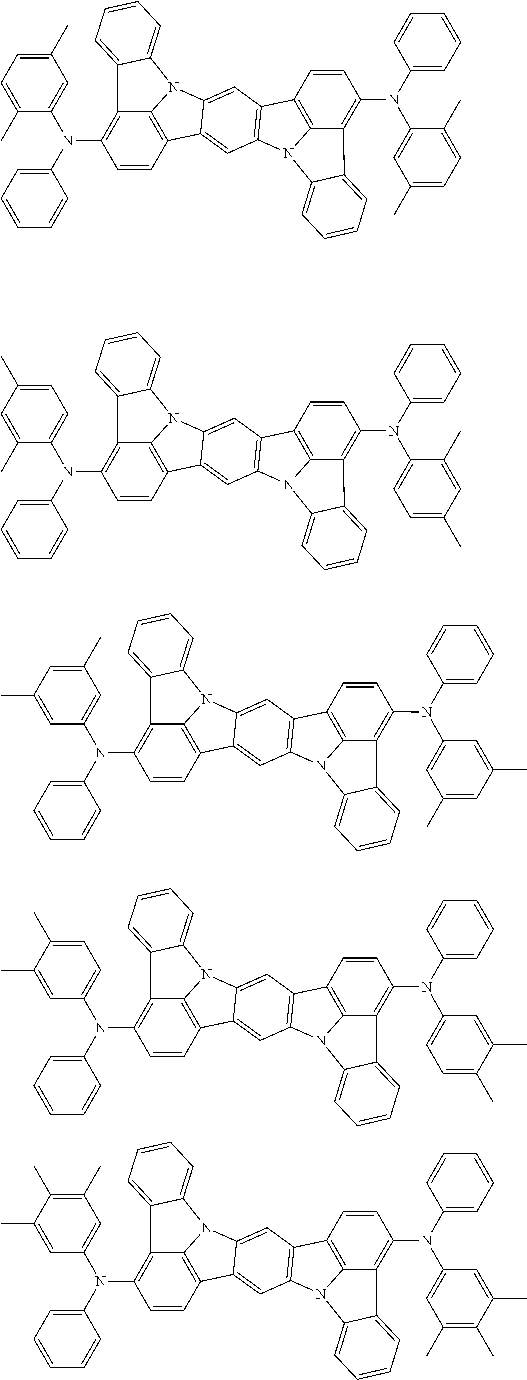

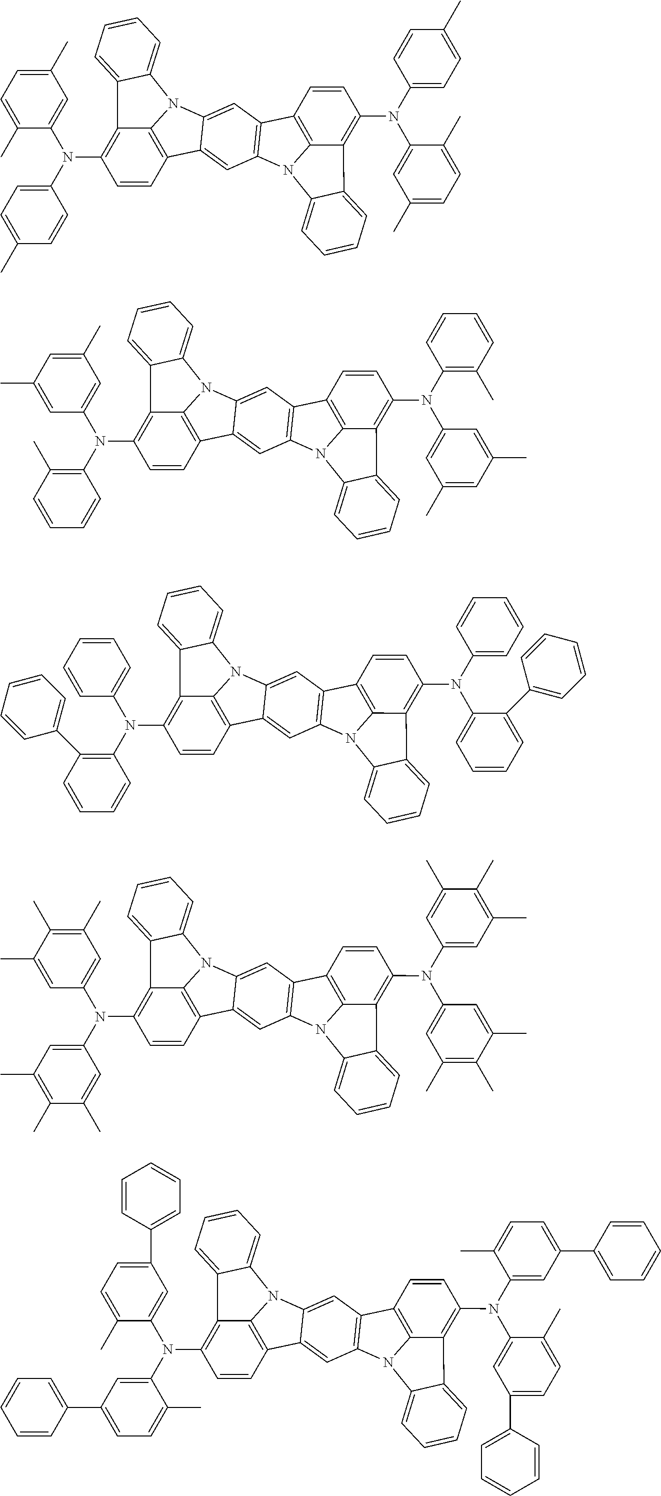

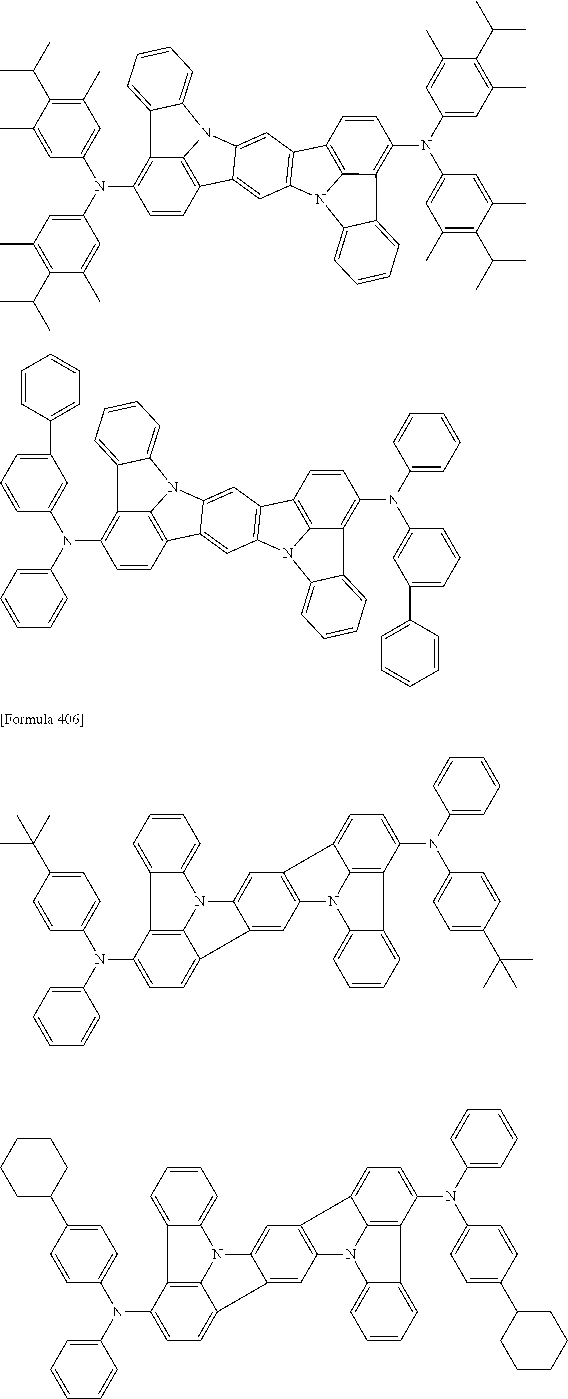

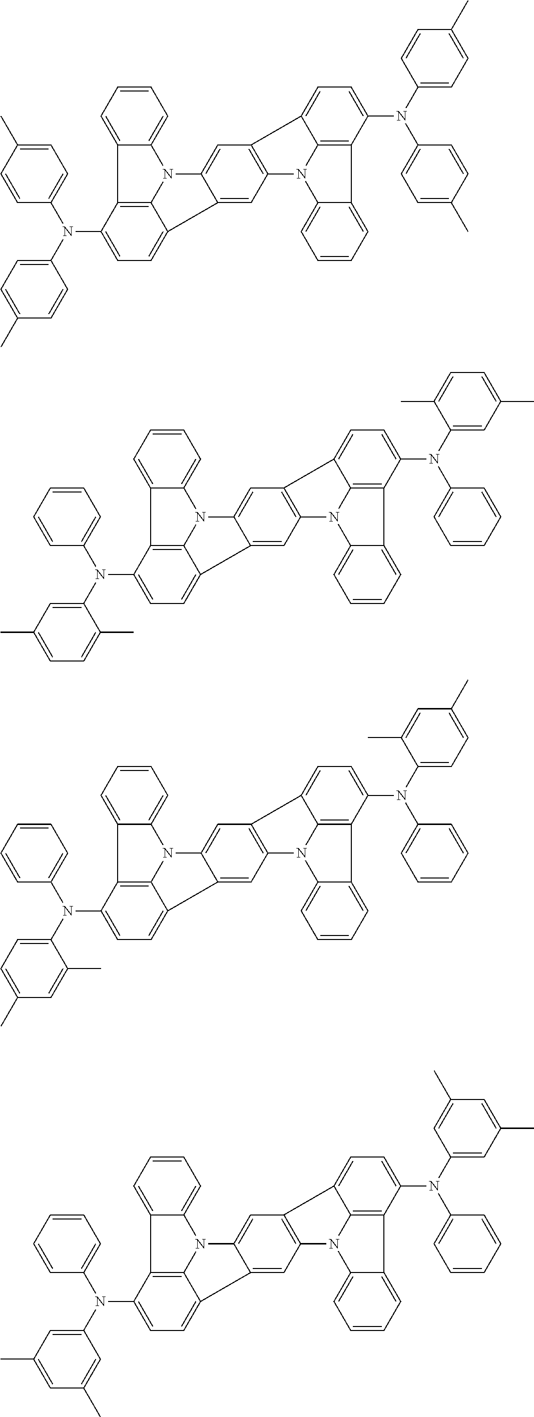

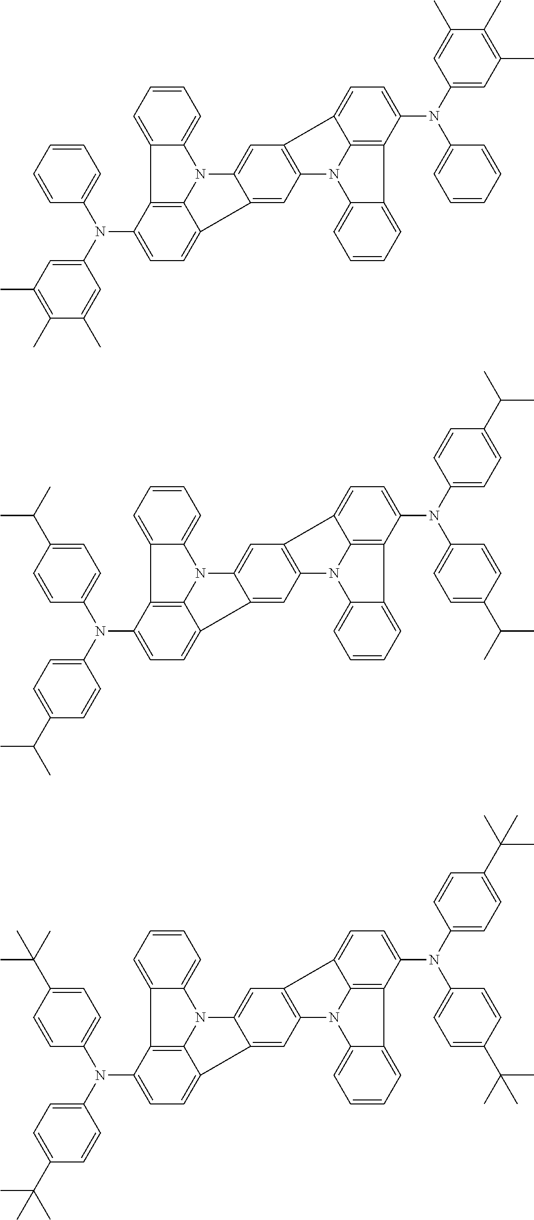

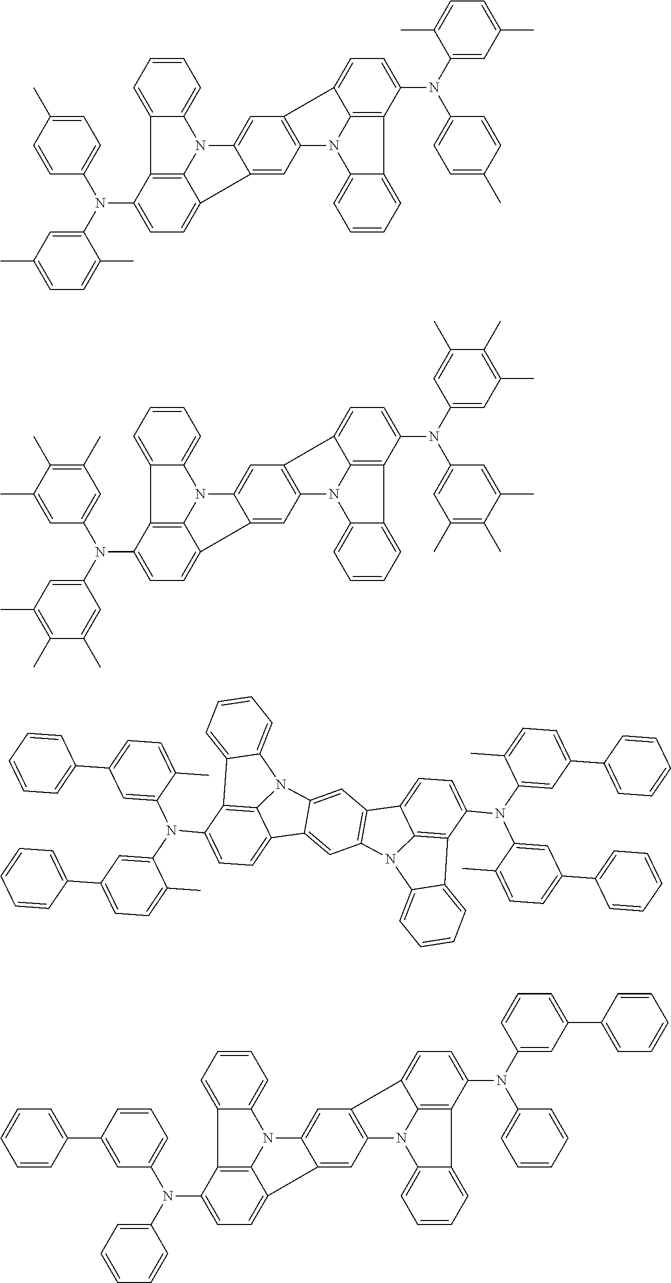

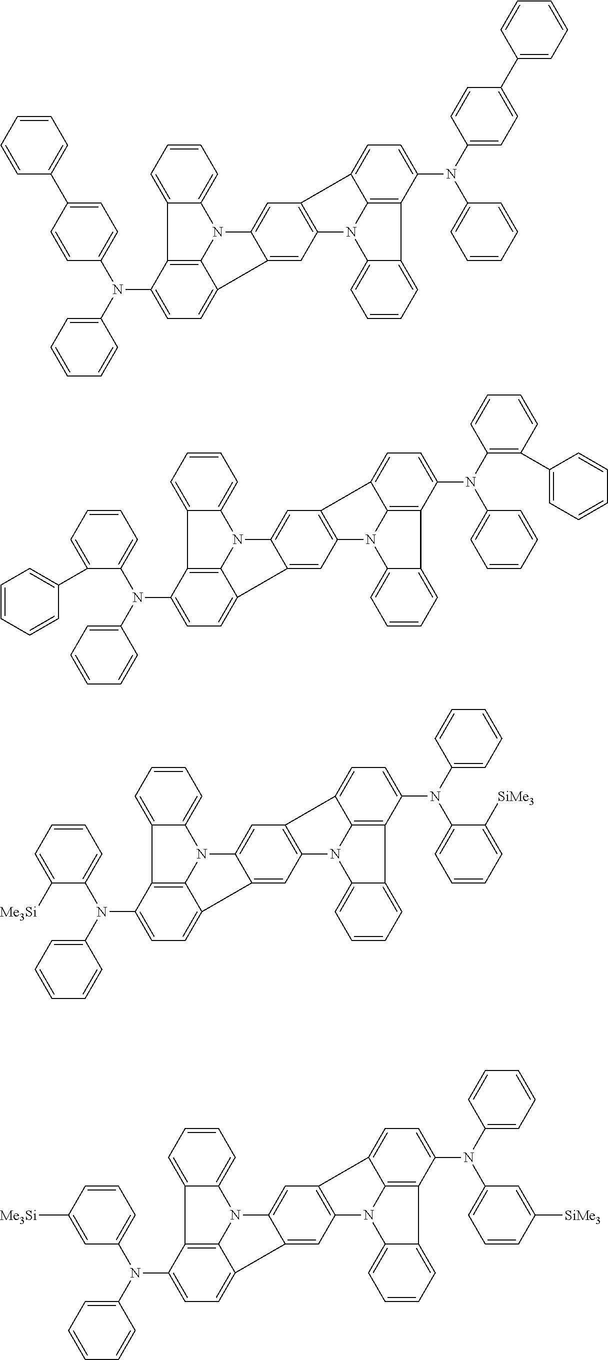 [SMILES: c1ccc(-c2ccc(N(c3ccccc3)c3ccc4c5cc6c(cc5n5c7ccccc7c3c45)c3ccc(N(c4ccccc4)c4ccc(-c5ccccc5)cc4)c4c5ccccc5n6c34)cc2)cc1 ; c1ccc(-c2ccccc2N(c2ccccc2)c2ccc3c4cc5c(cc4n4c6ccccc6c2c34)c2ccc(N(c3ccccc3)c3ccccc3-c3ccccc3)c3c4ccccc4n5c23)cc1 ; C[Si](C)(C)c1ccccc1N(c1ccccc1)c1ccc2c3cc4c(cc3n3c5ccccc5c1c23)c1ccc(N(c2ccccc2)c2ccccc2[Si](C)(C)C)c2c3ccccc3n4c12 ; C[Si](C)(C)c1cccc(N(c2ccccc2)c2ccc3c4cc5c(cc4n4c6ccccc6c2c34)c2ccc(N(c3ccccc3)c3cccc([Si](C)(C)C)c3)c3c4ccccc4n5c23)c1]

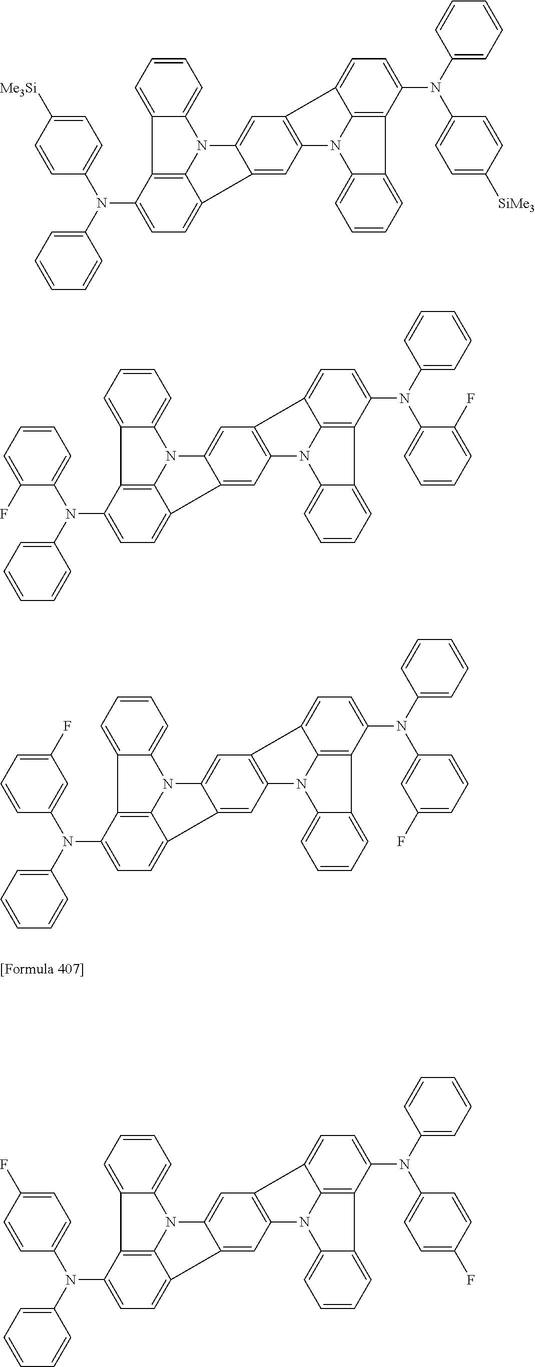 [SMILES: C[Si](C)(C)c1ccc(N(c2ccccc2)c2ccc3c4cc5c(cc4n4c6ccccc6c2c34)c2ccc(N(c3ccccc3)c3ccc([Si](C)(C)C)cc3)c3c4ccccc4n5c23)cc1 ; Fc1ccccc1N(c1ccccc1)c1ccc2c3cc4c(cc3n3c5ccccc5c1c23)c1ccc(N(c2ccccc2)c2ccccc2F)c2c3ccccc3n4c12 ; Fc1cccc(N(c2ccccc2)c2ccc3c4cc5c(cc4n4c6ccccc6c2c34)c2ccc(N(c3ccccc3)c3cccc(F)c3)c3c4ccccc4n5c23)c1 ; Fc1ccc(N(c2ccccc2)c2ccc3c4cc5c(cc4n4c6ccccc6c2c34)c2ccc(N(c3ccccc3)c3ccc(F)cc3)c3c4ccccc4n5c23)cc1]

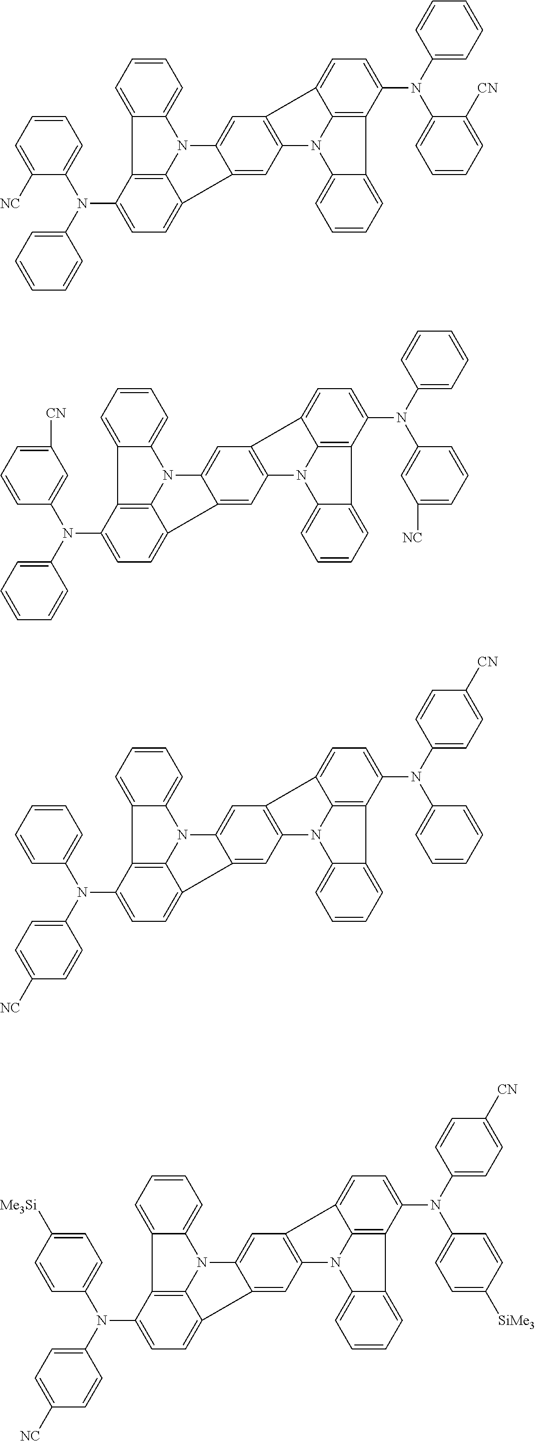 [SMILES: N#Cc1ccccc1N(c1ccccc1)c1ccc2c3cc4c(cc3n3c5ccccc5c1c23)c1ccc(N(c2ccccc2)c2ccccc2C#N)c2c3ccccc3n4c12 ; N#Cc1cccc(N(c2ccccc2)c2ccc3c4cc5c(cc4n4c6ccccc6c2c34)c2ccc(N(c3ccccc3)c3cccc(C#N)c3)c3c4ccccc4n5c23)c1 ; N#Cc1ccc(N(c2ccccc2)c2ccc3c4cc5c(cc4n4c6ccccc6c2c34)c2ccc(N(c3ccccc3)c3ccc(C#N)cc3)c3c4ccccc4n5c23)cc1 ; C[Si](C)(C)c1ccc(N(c2ccc(C#N)cc2)c2ccc3c4cc5c(cc4n4c6ccccc6c2c34)c2ccc(N(c3ccc(C#N)cc3)c3ccc([Si](C)(C)C)cc3)c3c4ccccc4n5c23)cc1]

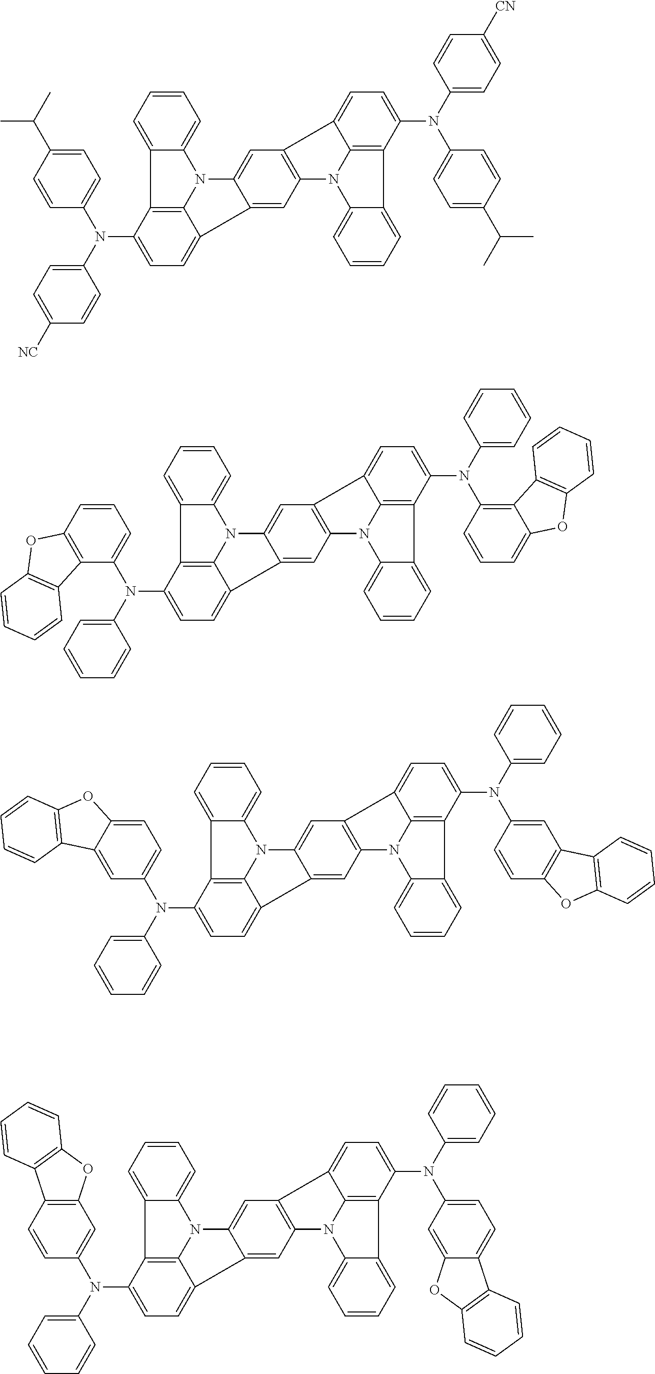 [SMILES: CC(C)c1ccc(N(c2ccc(C#N)cc2)c2ccc3c4cc5c(cc4n4c6ccccc6c2c34)c2ccc(N(c3ccc(C#N)cc3)c3ccc(C(C)C)cc3)c3c4ccccc4n5c23)cc1 ; c1ccc(N(c2cccc3oc4ccccc4c23)c2ccc3c4cc5c(cc4n4c6ccccc6c2c34)c2ccc(N(c3ccccc3)c3cccc4oc6ccccc6c34)c3c4ccccc4n5c23)cc1 ; c1ccc(N(c2ccc3oc4ccccc4c3c2)c2ccc3c4cc5c(cc4n4c6ccccc6c2c34)c2ccc(N(c3ccccc3)c3ccc4oc6ccccc6c4c3)c3c4ccccc4n5c23)cc1 ; c1ccc(N(c2ccc3c(c2)oc2ccccc23)c2ccc3c4cc5c(cc4n4c6ccccc6c2c34)c2ccc(N(c3ccccc3)c3ccc4c(c3)oc3ccccc34)c3c4ccccc4n5c23)cc1]

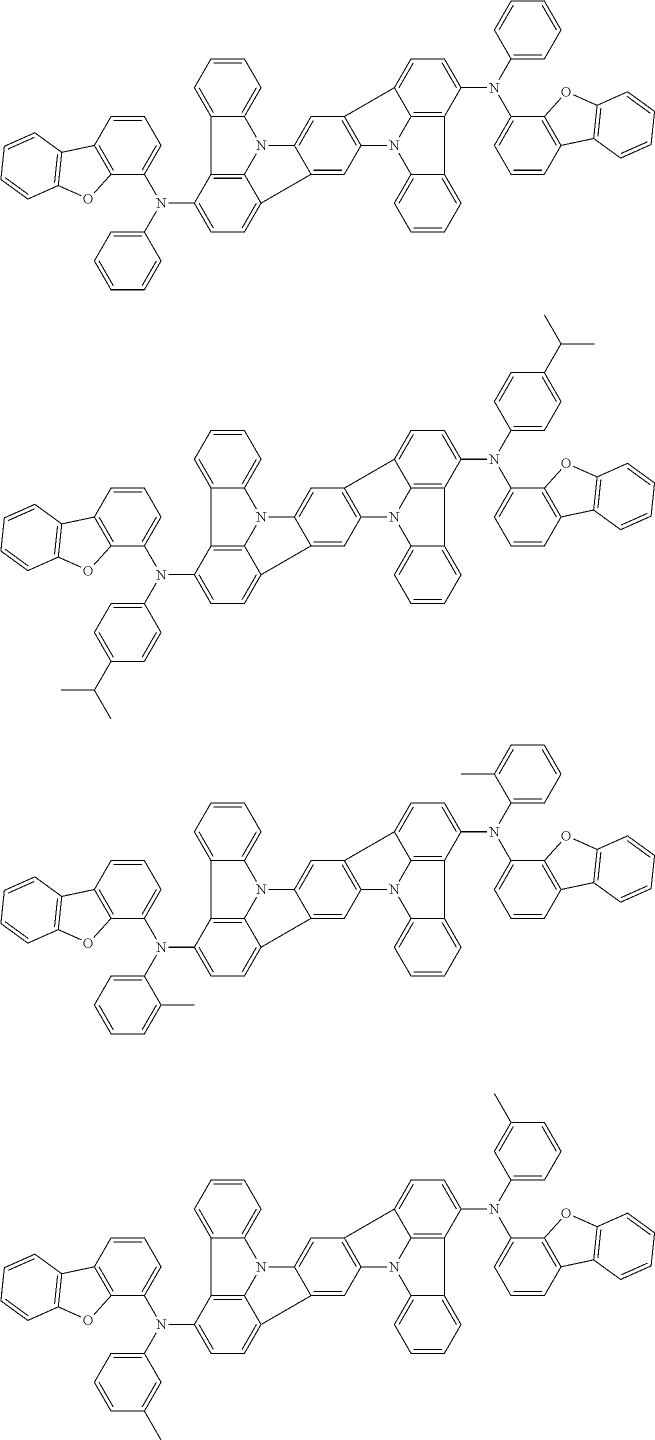 [SMILES: c1ccc(N(c2cccc3c2oc2ccccc23)c2ccc3c4cc5c(cc4n4c6ccccc6c2c34)c2ccc(N(c3ccccc3)c3cccc4c3oc3ccccc34)c3c4ccccc4n5c23)cc1 ; CC(C)c1ccc(N(c2cccc3c2oc2ccccc23)c2ccc3c4cc5c(cc4n4c6ccccc6c2c34)c2ccc(N(c3ccc(C(C)C)cc3)c3cccc4c3oc3ccccc34)c3c4ccccc4n5c23)cc1 ; Cc1ccccc1N(c1cccc2c1oc1ccccc12)c1ccc2c3cc4c(cc3n3c5ccccc5c1c23)c1ccc(N(c2ccccc2C)c2cccc3c2oc2ccccc23)c2c3ccccc3n4c12 ; Cc1cccc(N(c2cccc3c2oc2ccccc23)c2ccc3c4cc5c(cc4n4c6ccccc6c2c34)c2ccc(N(c3cccc(C)c3)c3cccc4c3oc3ccccc34)c3c4ccccc4n5c23)c1]

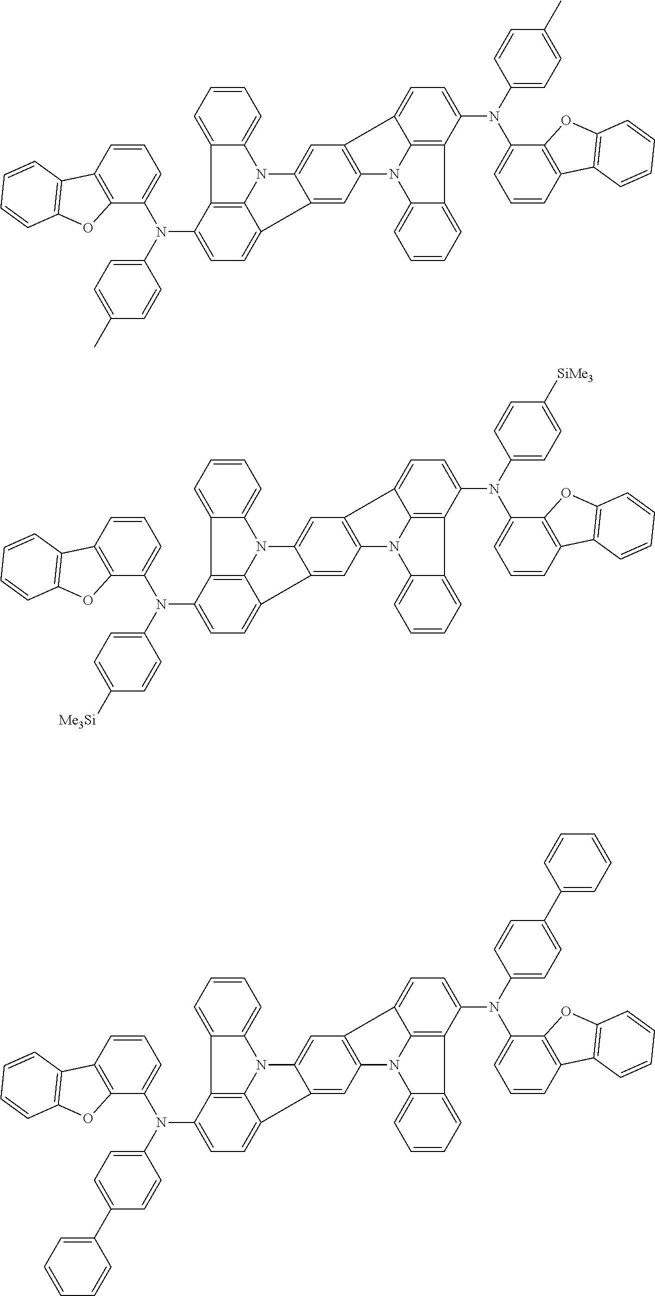 [SMILES: Cc1ccc(N(c2cccc3c2oc2ccccc23)c2ccc3c4cc5c(cc4n4c6ccccc6c2c34)c2ccc(N(c3ccc(C)cc3)c3cccc4c3oc3ccccc34)c3c4ccccc4n5c23)cc1 ; C[Si](C)(C)c1ccc(N(c2cccc3c2oc2ccccc23)c2ccc3c4cc5c(cc4n4c6ccccc6c2c34)c2ccc(N(c3ccc([Si](C)(C)C)cc3)c3cccc4c3oc3ccccc34)c3c4ccccc4n5c23)cc1 ; c1ccc(-c2ccc(N(c3cccc4c3oc3ccccc34)c3ccc4c5cc6c(cc5n5c7ccccc7c3c45)c3ccc(N(c4ccc(-c5ccccc5)cc4)c4cccc5c4oc4ccccc45)c4c5ccccc5n6c34)cc2)cc1]

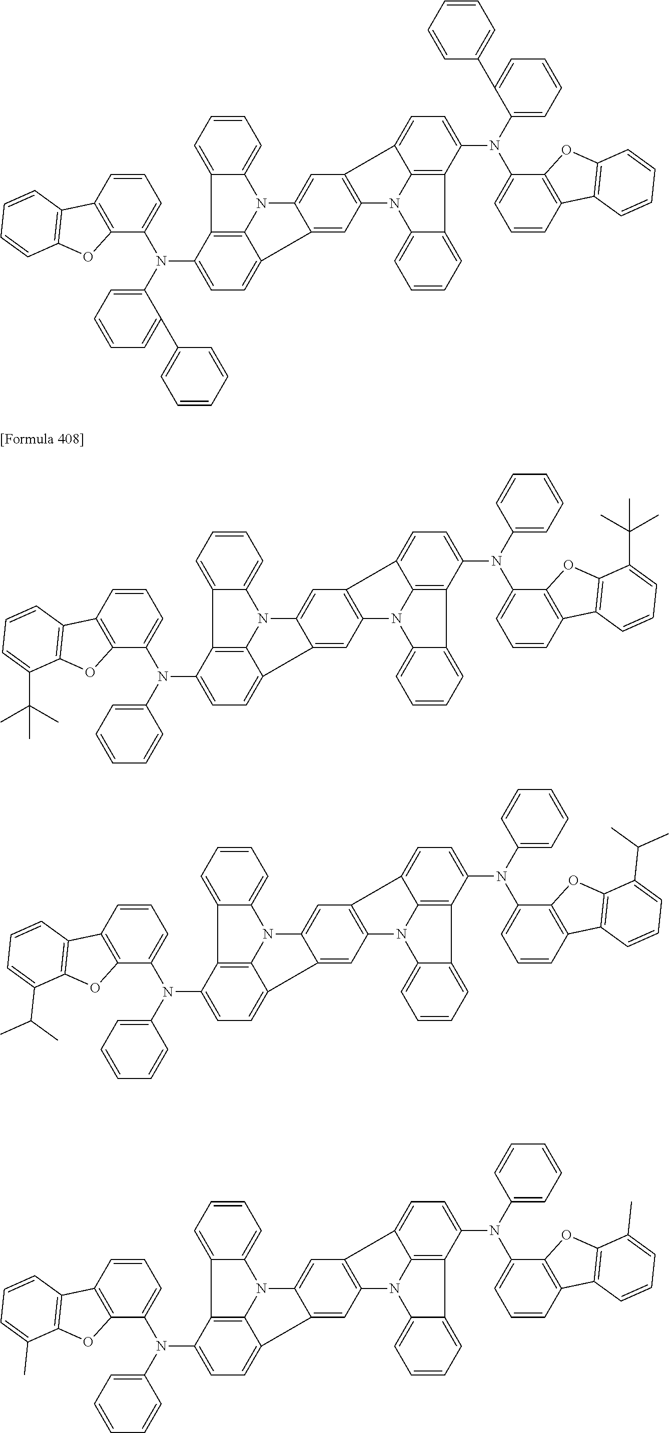

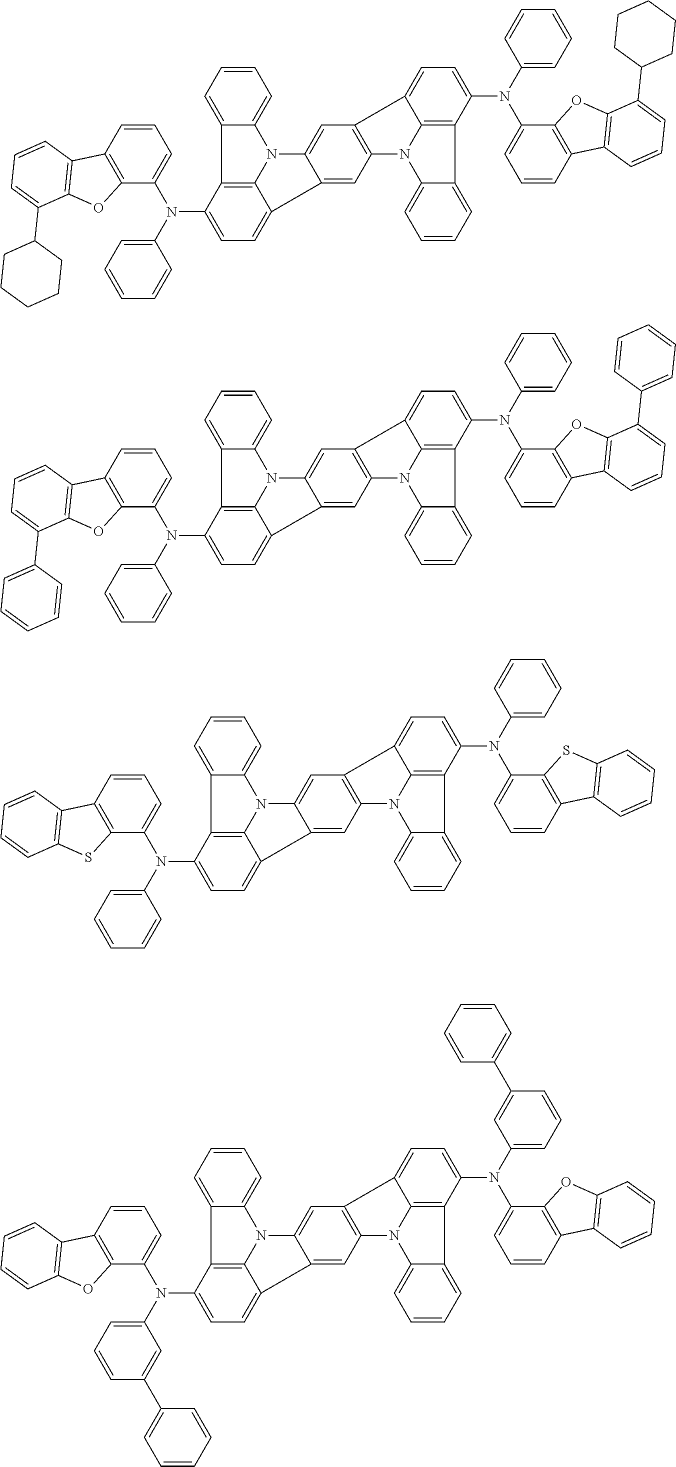

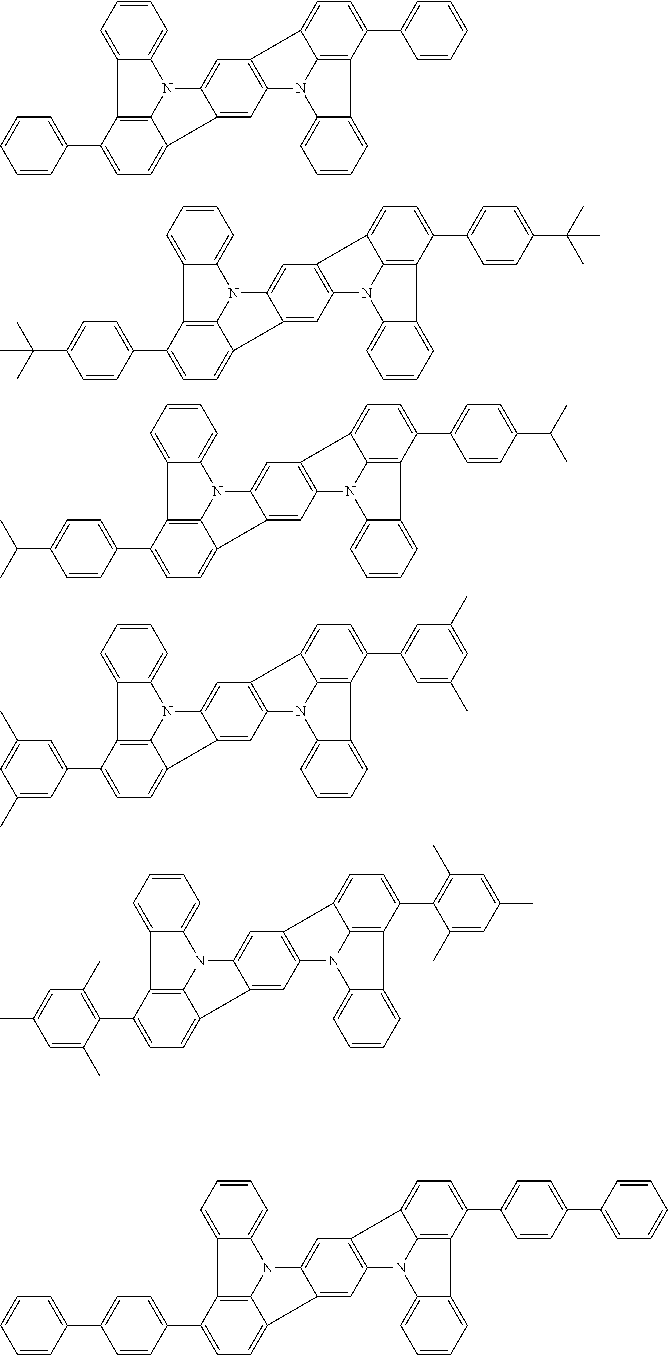

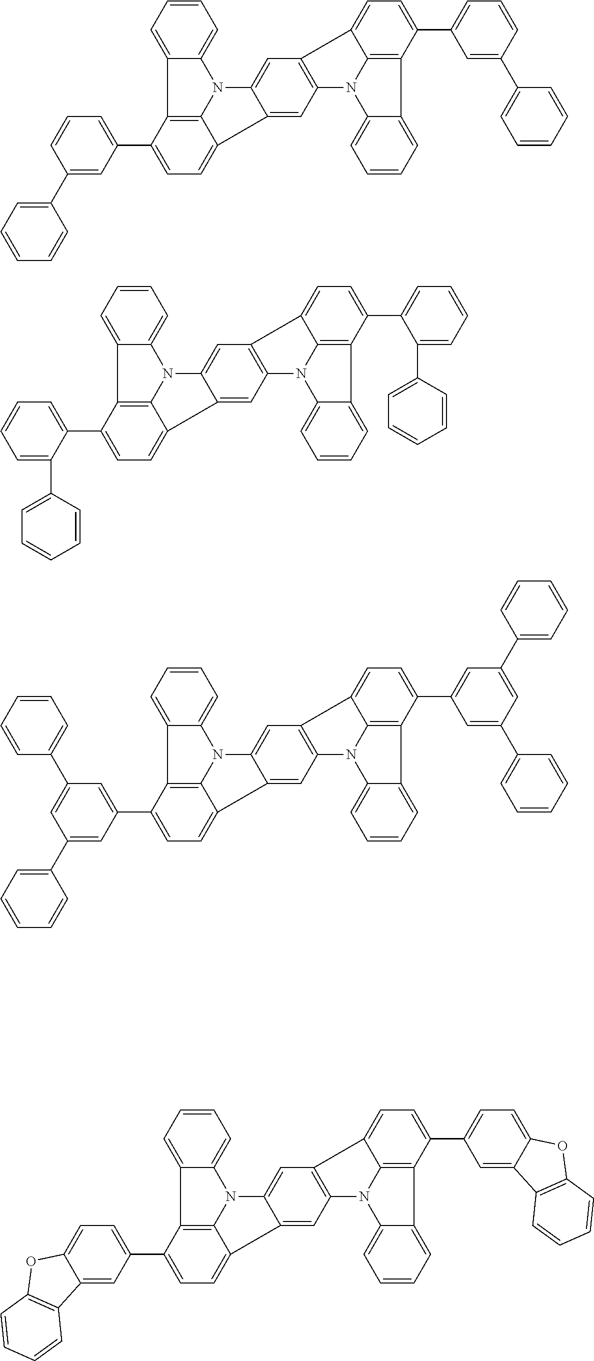 [SMILES: c1ccc(-c2cccc(-c3ccc4c5cc6c(cc5n5c7ccccc7c3c45)c3ccc(-c4cccc(-c5ccccc5)c4)c4c5ccccc5n6c34)c2)cc1 ; c1ccc(-c2ccccc2-c2ccc3c4cc5c(cc4n4c6ccccc6c2c34)c2ccc(-c3ccccc3-c3ccccc3)c3c4ccccc4n5c23)cc1 ; c1ccc(-c2cc(-c3ccccc3)cc(-c3ccc4c5cc6c(cc5n5c7ccccc7c3c45)c3ccc(-c4cc(-c5ccccc5)cc(-c5ccccc5)c4)c4c5ccccc5n6c34)c2)cc1 ; c1ccc2c(c1)oc1ccc(-c3ccc4c5cc6c(cc5n5c7ccccc7c3c45)c3ccc(-c4ccc5oc7ccccc7c5c4)c4c5ccccc5n6c34)cc12]

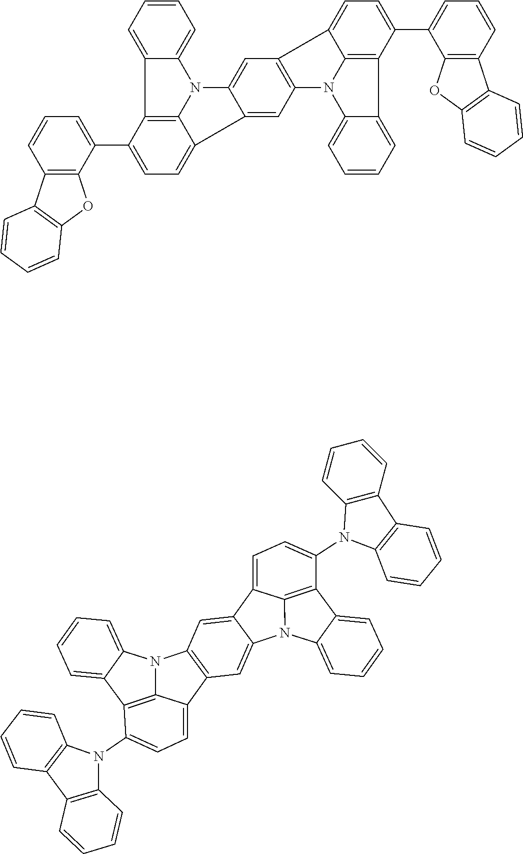 [SMILES: c1ccc2c(c1)oc1c(-c3ccc4c5cc6c(cc5n5c7ccccc7c3c45)c3ccc(-c4cccc5c4oc4ccccc45)c4c5ccccc5n6c34)cccc12 ; c1ccc2c(c1)c1ccccc1n2-c1ccc2c3cc4c(cc3n3c5ccccc5c1c23)c1ccc(-n2c3ccccc3c3ccccc32)c2c3ccccc3n4c12]

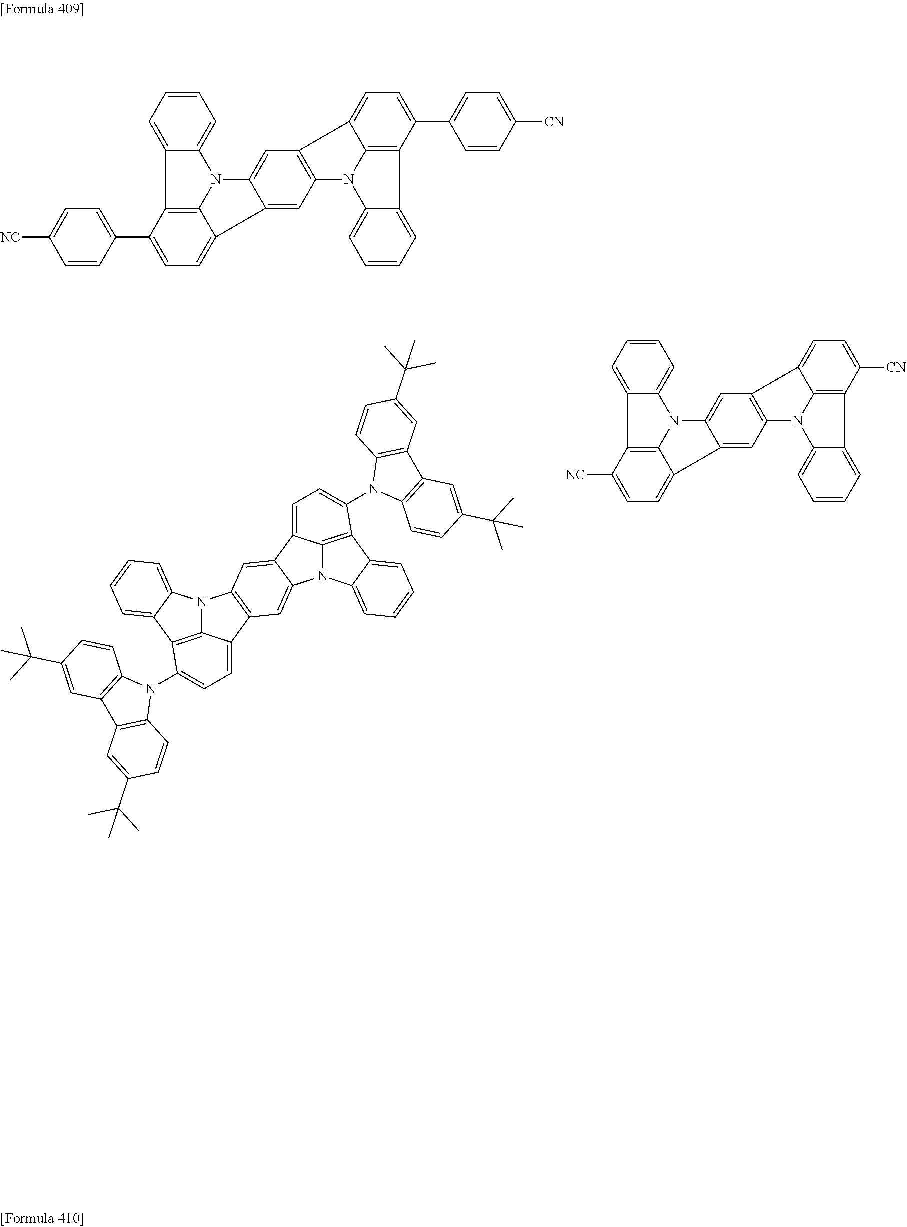 [SMILES: N#Cc1ccc(-c2ccc3c4cc5c(cc4n4c6ccccc6c2c34)c2ccc(-c3ccc(C#N)cc3)c3c4ccccc4n5c23)cc1 ; CC(C)(C)c1ccc2c(c1)c1cc(C(C)(C)C)ccc1n2-c1ccc2c3cc4c(cc3n3c5ccccc5c1c23)c1ccc(-n2c3ccc(C(C)(C)C)cc3c3cc(C(C)(C)C)ccc32)c2c3ccccc3n4c12 ; N#Cc1ccc2c3cc4c(cc3n3c5ccccc5c1c23)c1ccc(C#N)c2c3ccccc3n4c12]

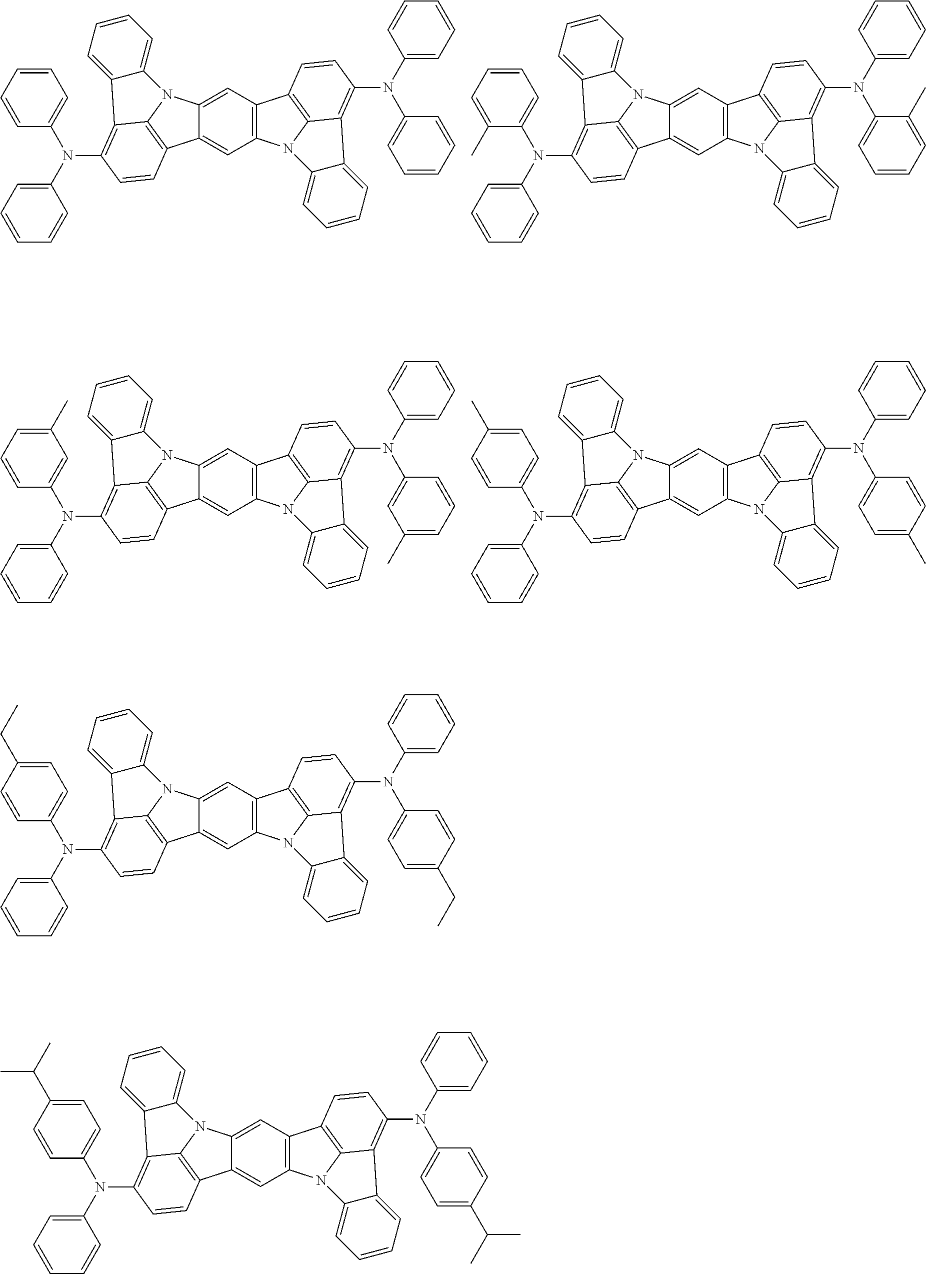

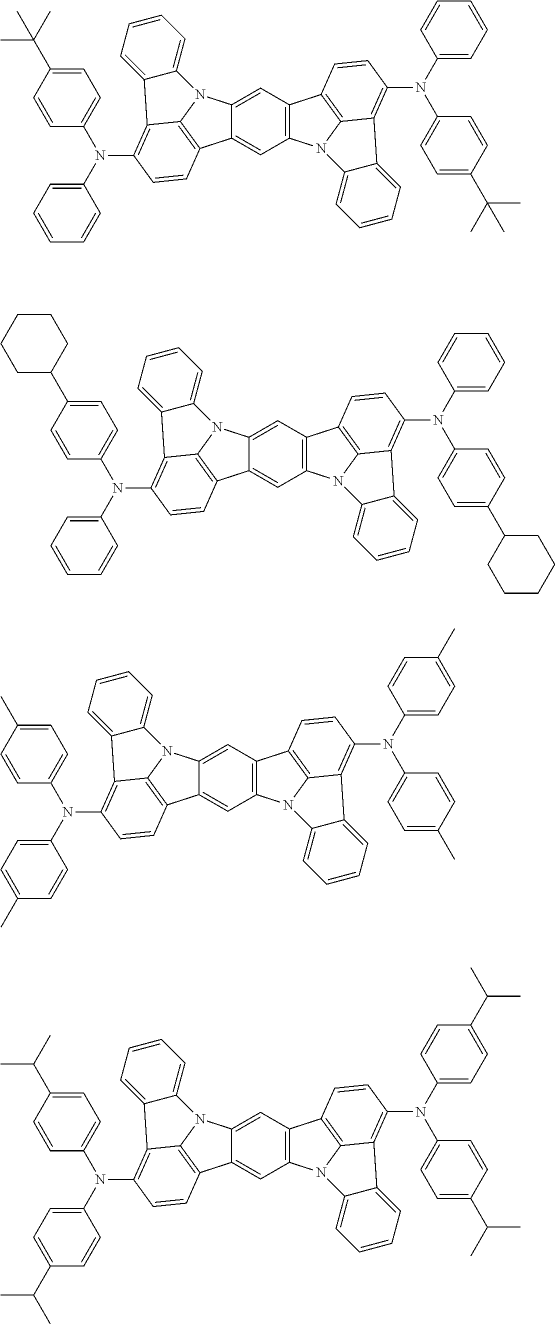

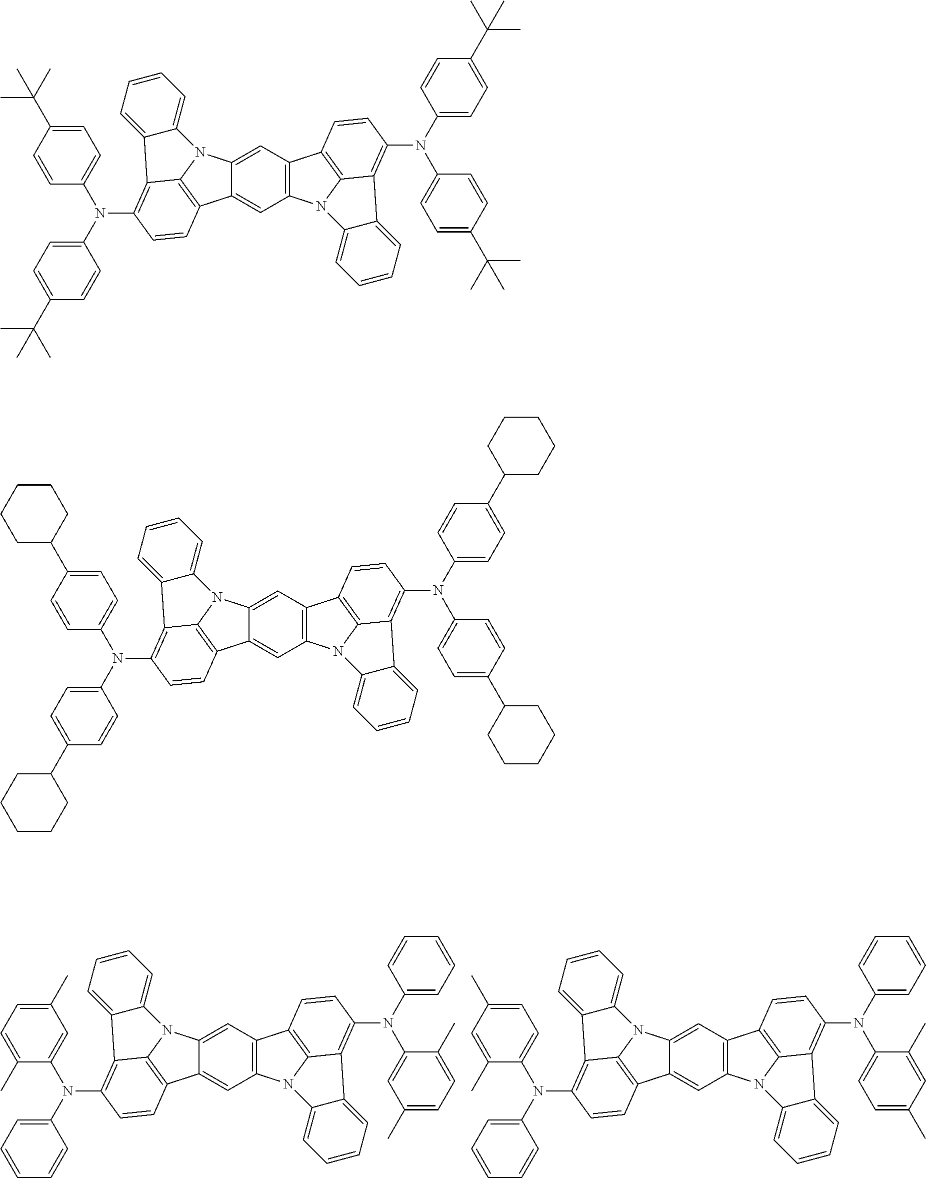

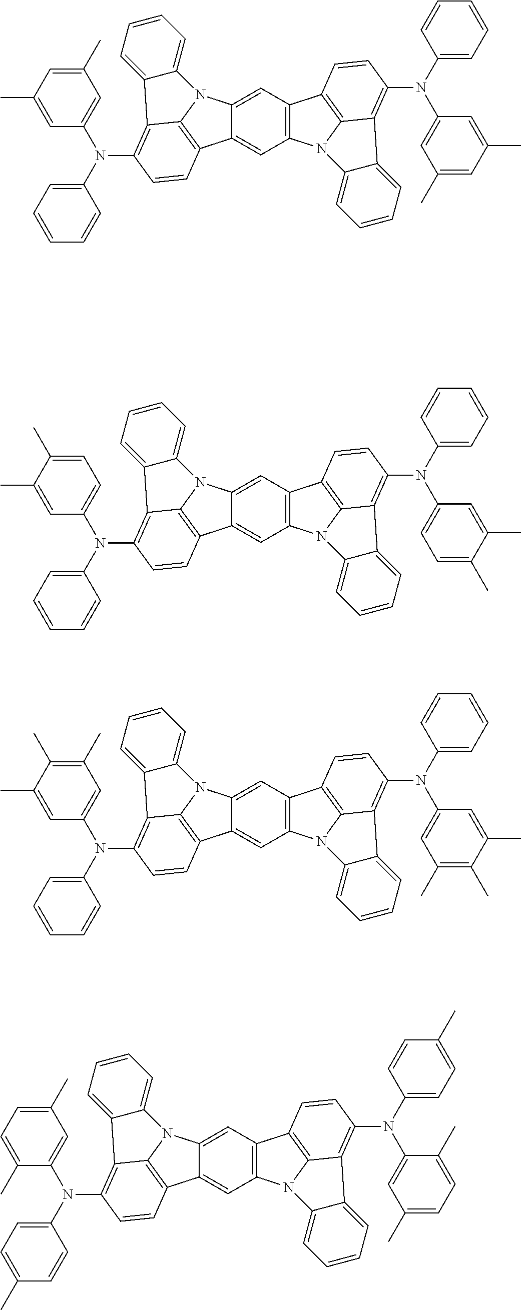

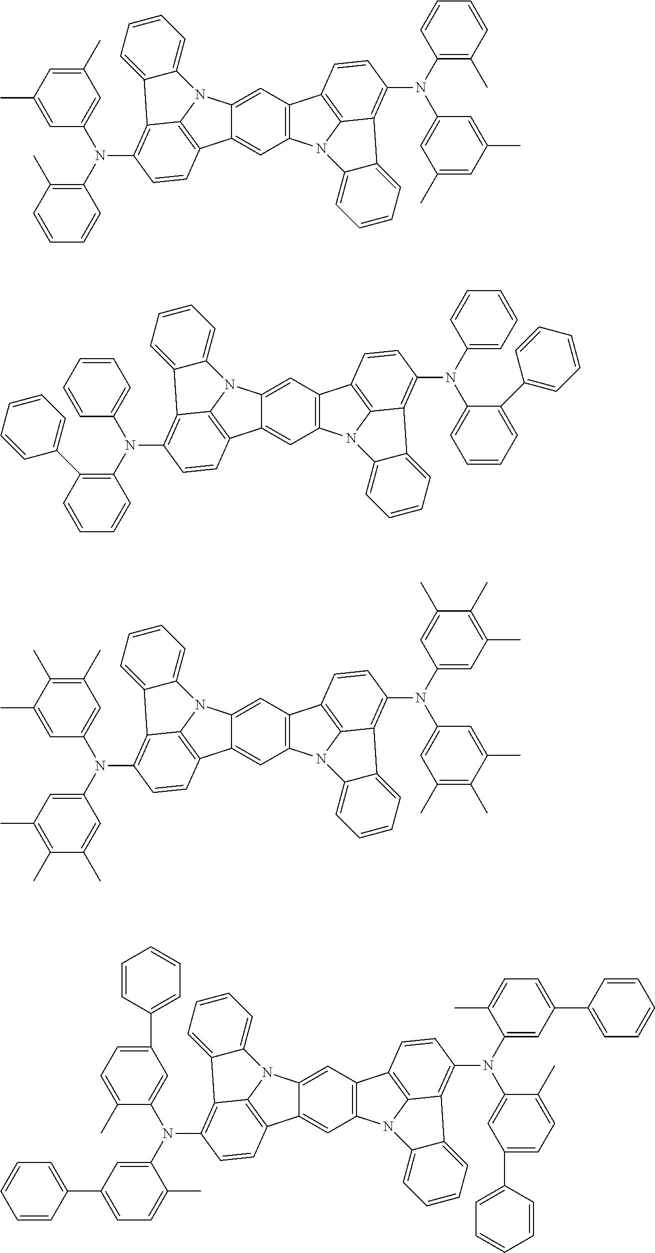

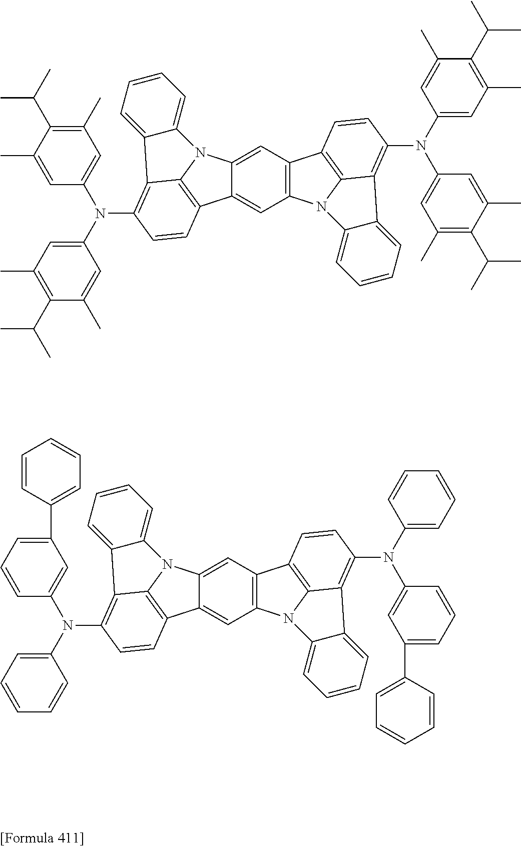 [SMILES: Cc1cc(N(c2cc(C)c(C(C)C)c(C)c2)c2ccc3c4cc5c(cc4n4c6ccccc6c2c34)c2ccc(N(c3cc(C)c(C(C)C)c(C)c3)c3cc(C)c(C(C)C)c(C)c3)c3c4ccccc4n5c23)cc(C)c1C(C)C ; c1ccc(-c2cccc(N(c3ccccc3)c3ccc4c5cc6c(cc5n5c7ccccc7c3c45)c3ccc(N(c4ccccc4)c4cccc(-c5ccccc5)c4)c4c5ccccc5n6c34)c2)cc1]

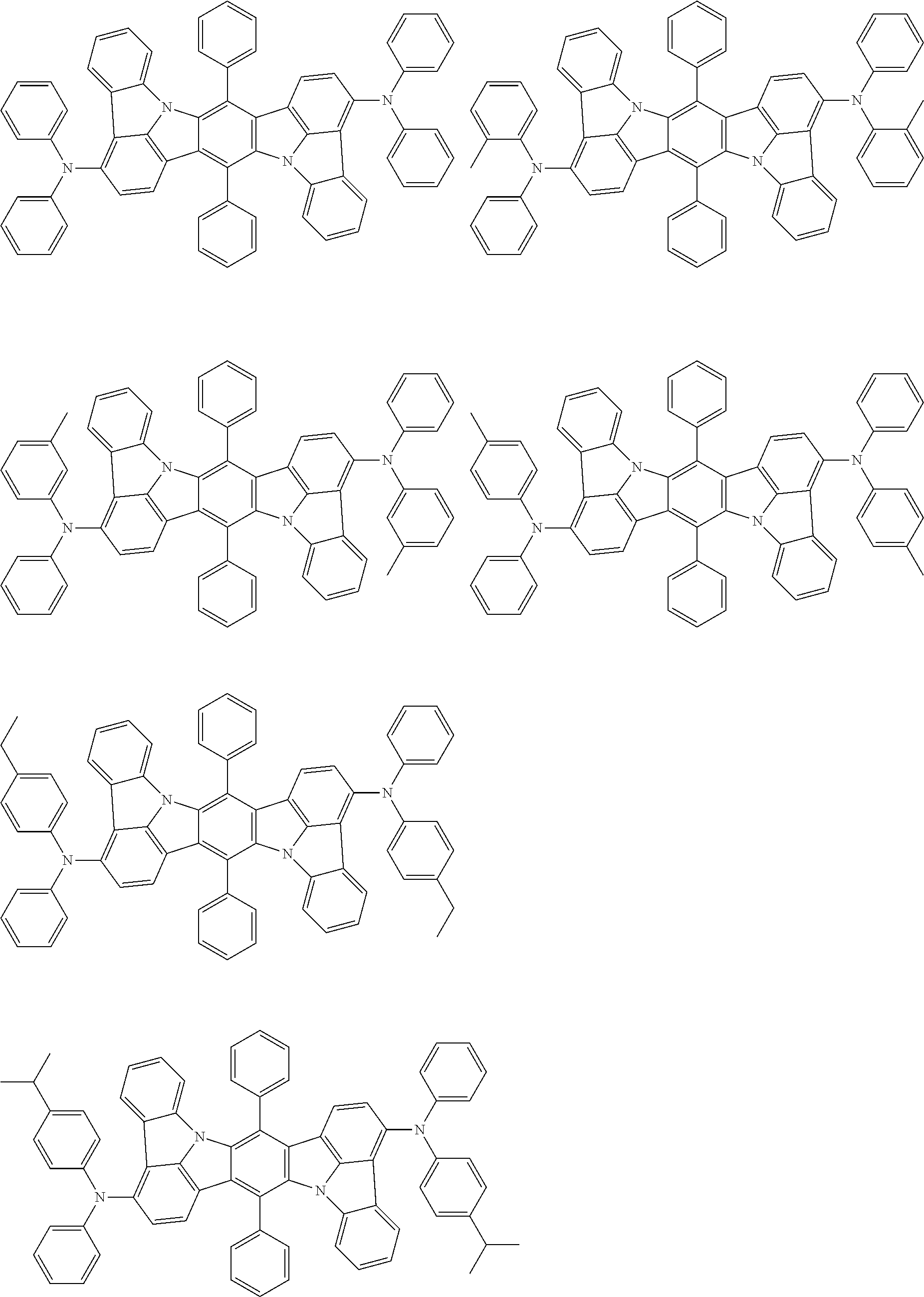

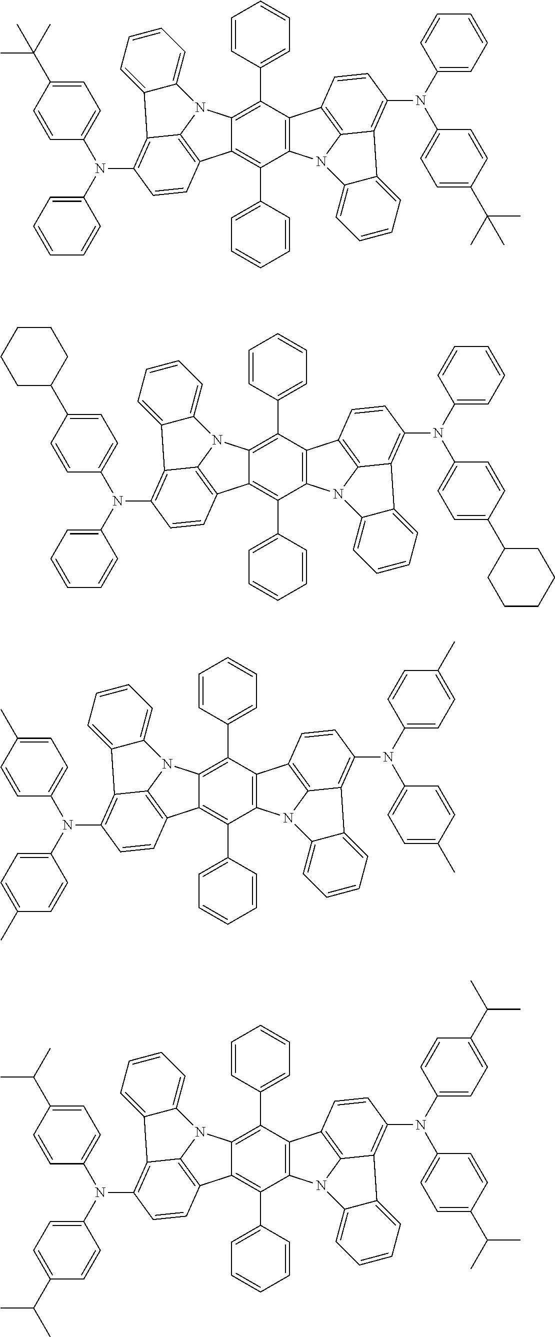

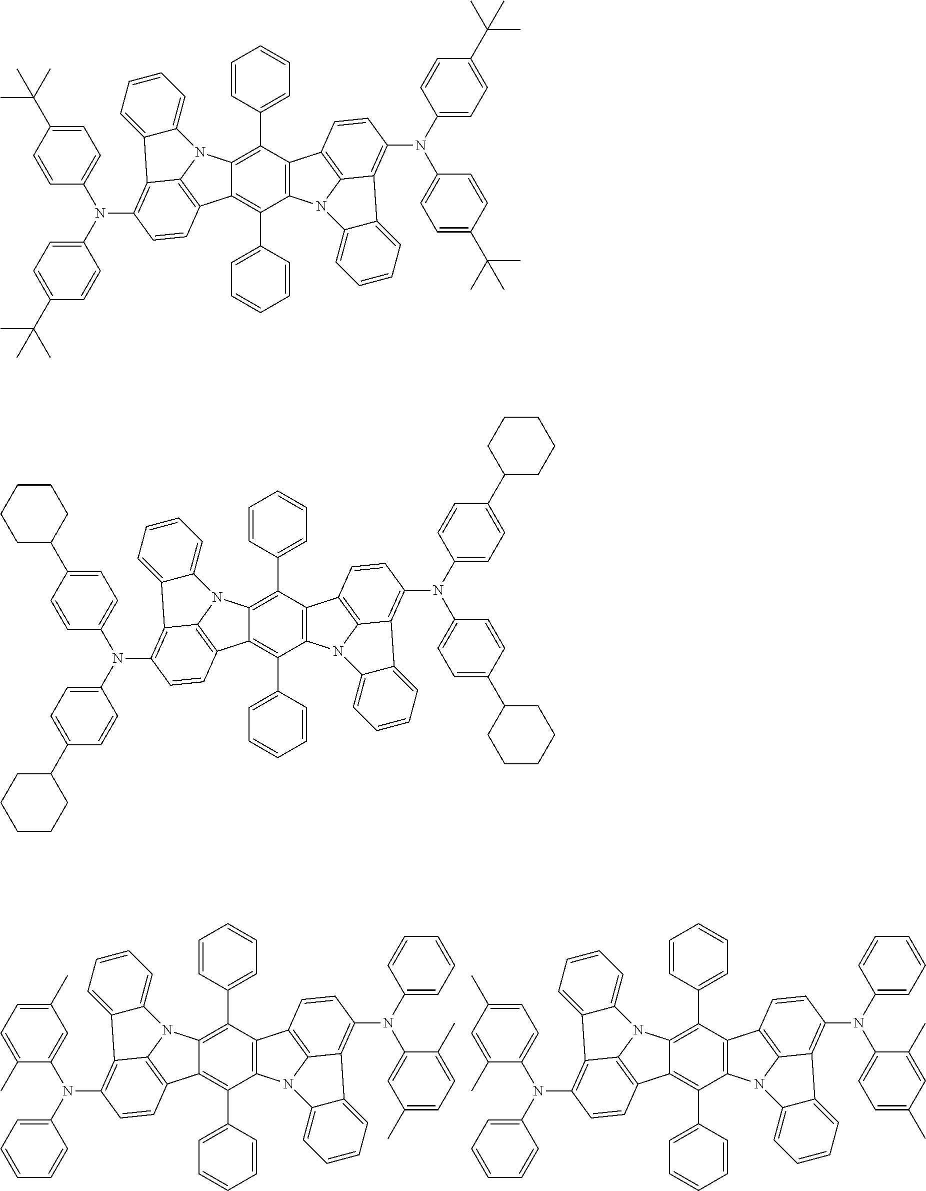

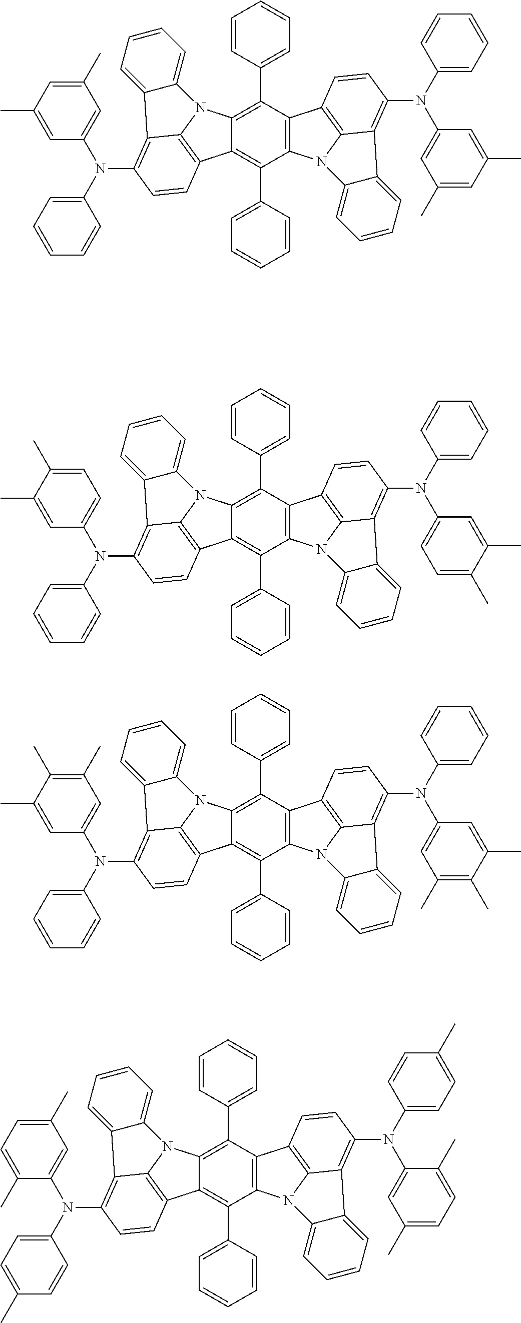

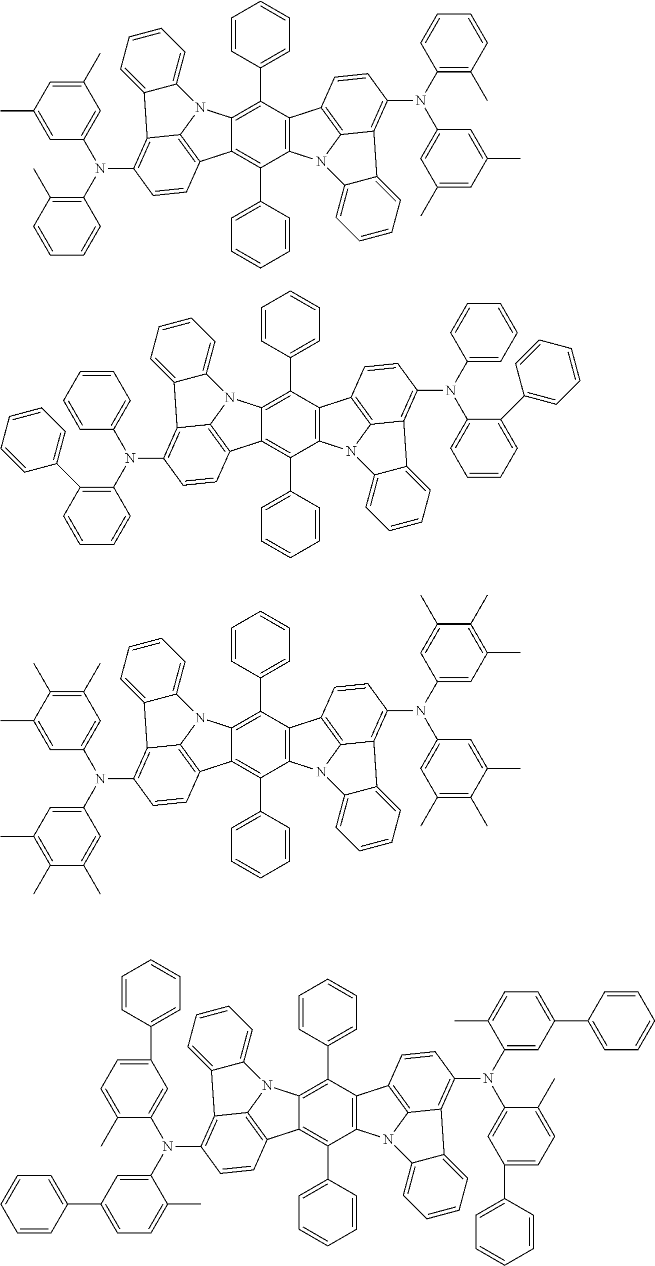

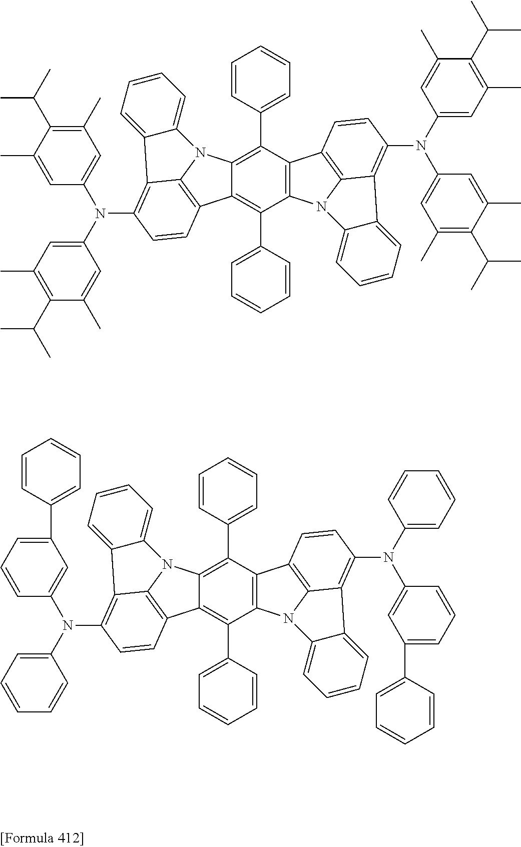 [SMILES: Cc1cc(N(c2cc(C)c(C(C)C)c(C)c2)c2ccc3c4c(-c5ccccc5)c5c(c(-c6ccccc6)c4n4c6ccccc6c2c34)c2ccc(N(c3cc(C)c(C(C)C)c(C)c3)c3cc(C)c(C(C)C)c(C)c3)c3c4ccccc4n5c23)cc(C)c1C(C)C ; c1ccc(-c2cccc(N(c3ccccc3)c3ccc4c5c(-c6ccccc6)c6c(c(-c7ccccc7)c5n5c7ccccc7c3c45)c3ccc(N(c4ccccc4)c4cccc(-c5ccccc5)c4)c4c5ccccc5n6c34)c2)cc1]

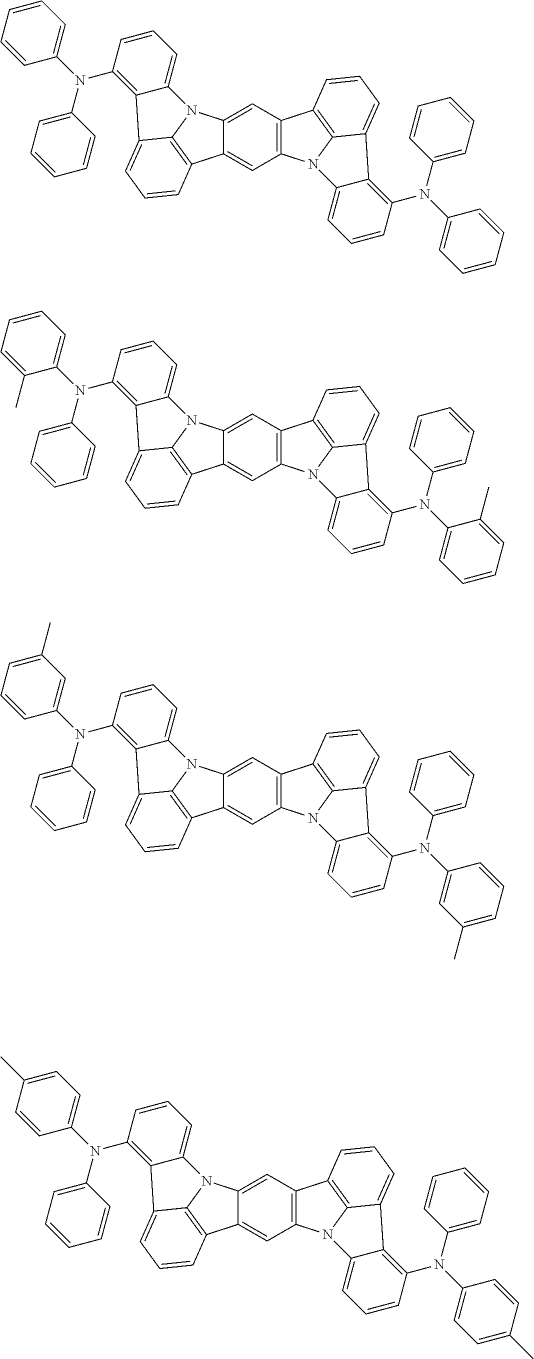

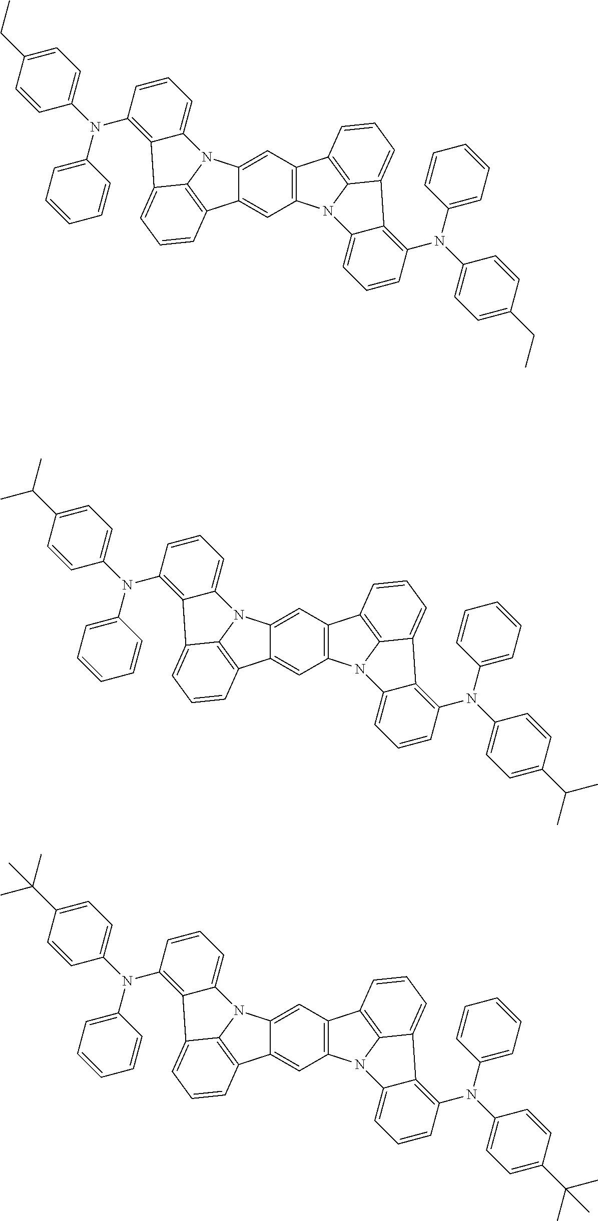

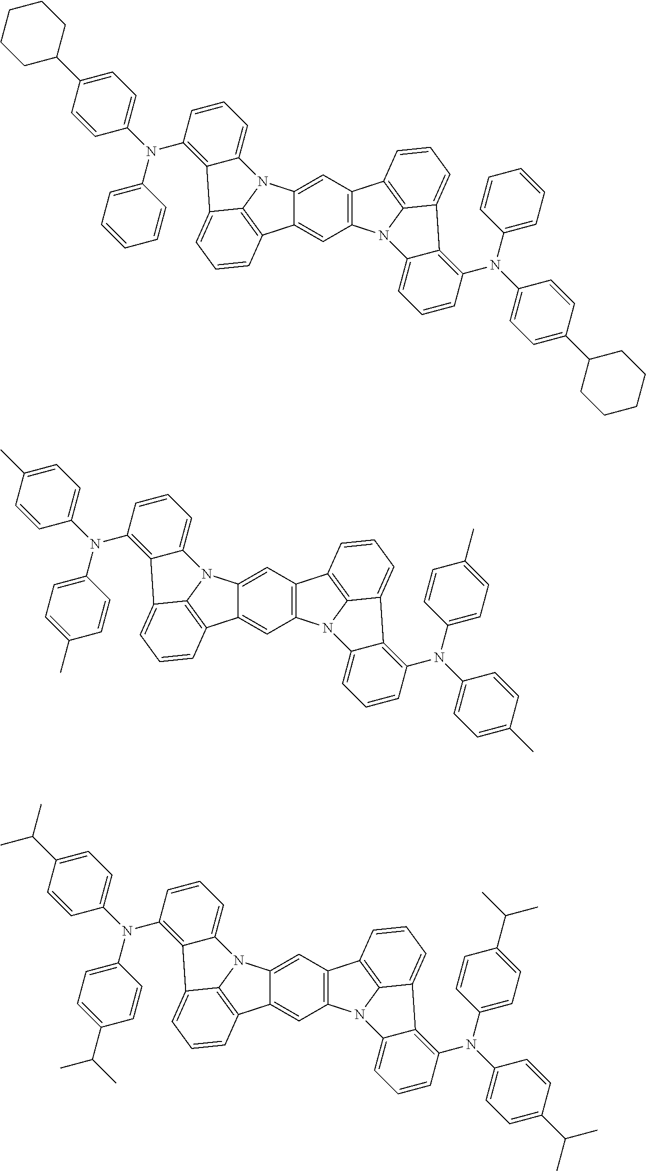

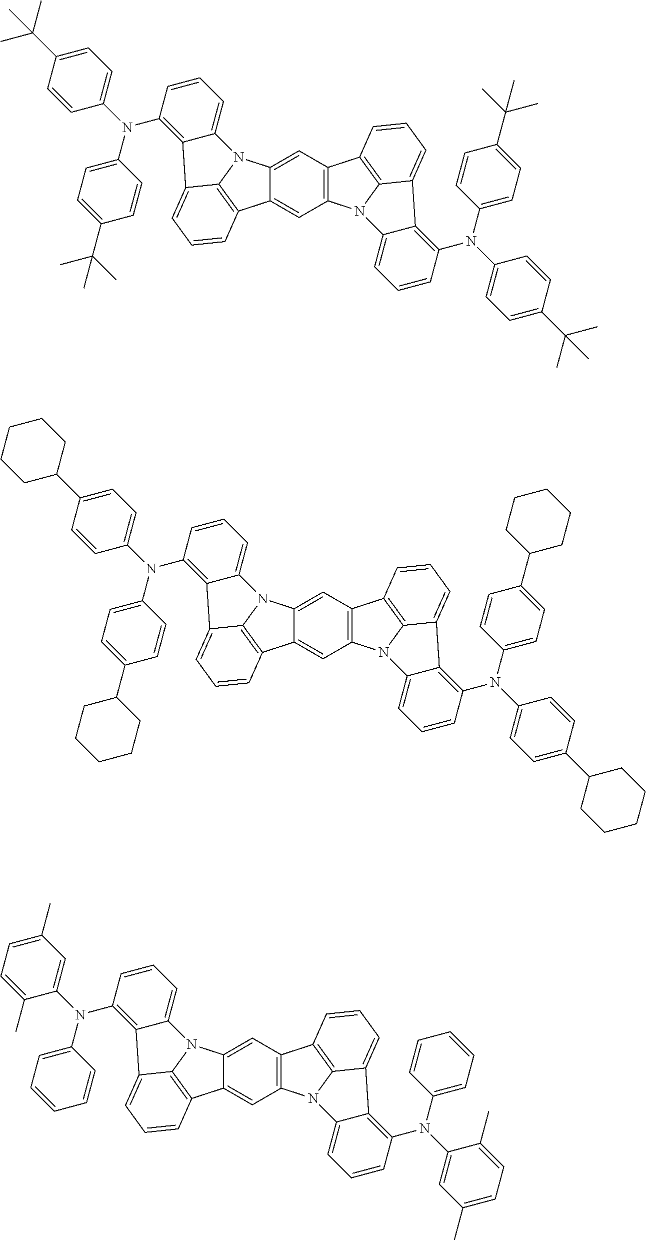 [SMILES: CC(C)(C)c1ccc(N(c2ccc(C(C)(C)C)cc2)c2cccc3c2c2cccc4c5cc6c(cc5n3c42)c2cccc3c4c(N(c5ccc(C(C)(C)C)cc5)c5ccc(C(C)(C)C)cc5)cccc4n6c23)cc1 ; c1cc2c3cc4c(cc3n3c5cccc(N(c6ccc(C7CCCCC7)cc6)c6ccc(C7CCCCC7)cc6)c5c(c1)c23)c1cccc2c3c(N(c5ccc(C6CCCCC6)cc5)c5ccc(C6CCCCC6)cc5)cccc3n4c12 ; Cc1ccc(C)c(N(c2ccccc2)c2cccc3c2c2cccc4c5cc6c(cc5n3c42)c2cccc3c4c(N(c5ccccc5)c5cc(C)ccc5C)cccc4n6c23)c1]

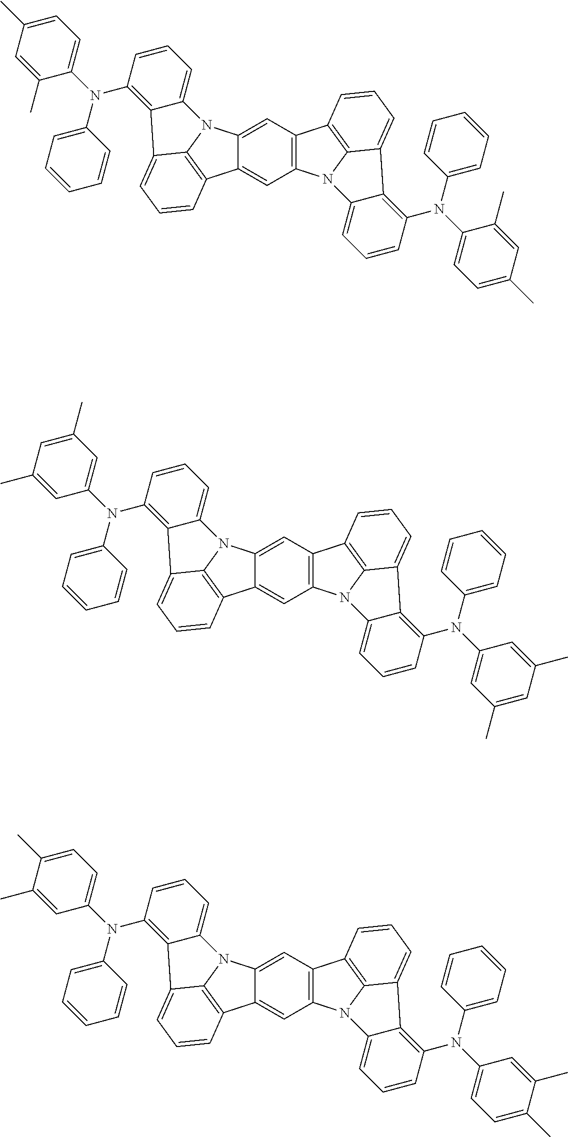

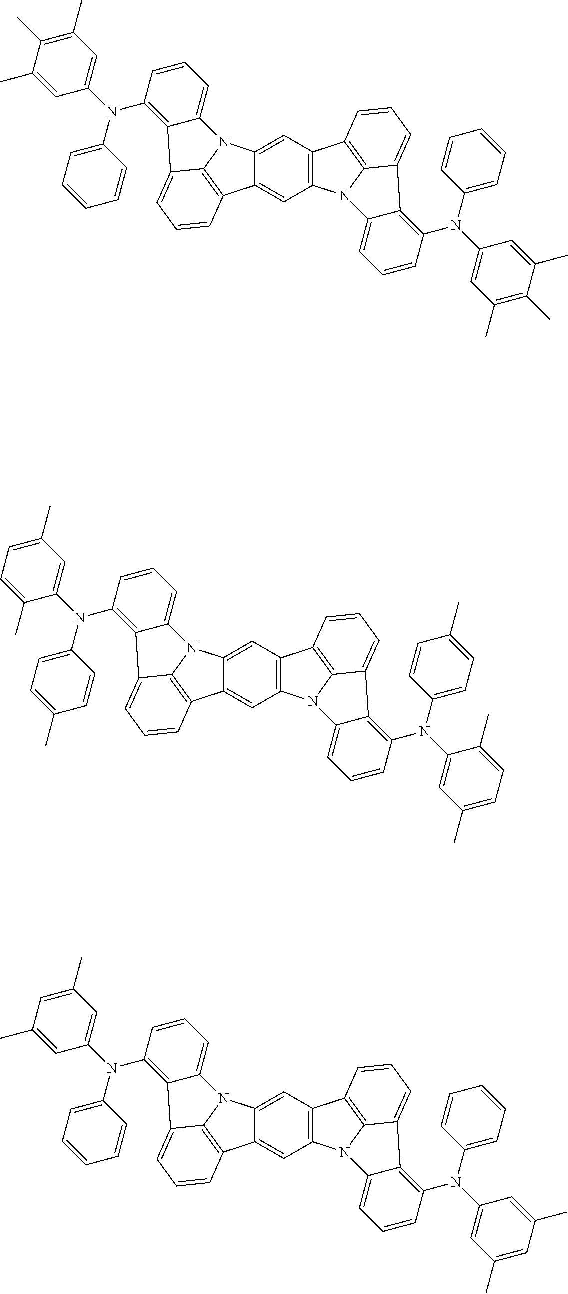

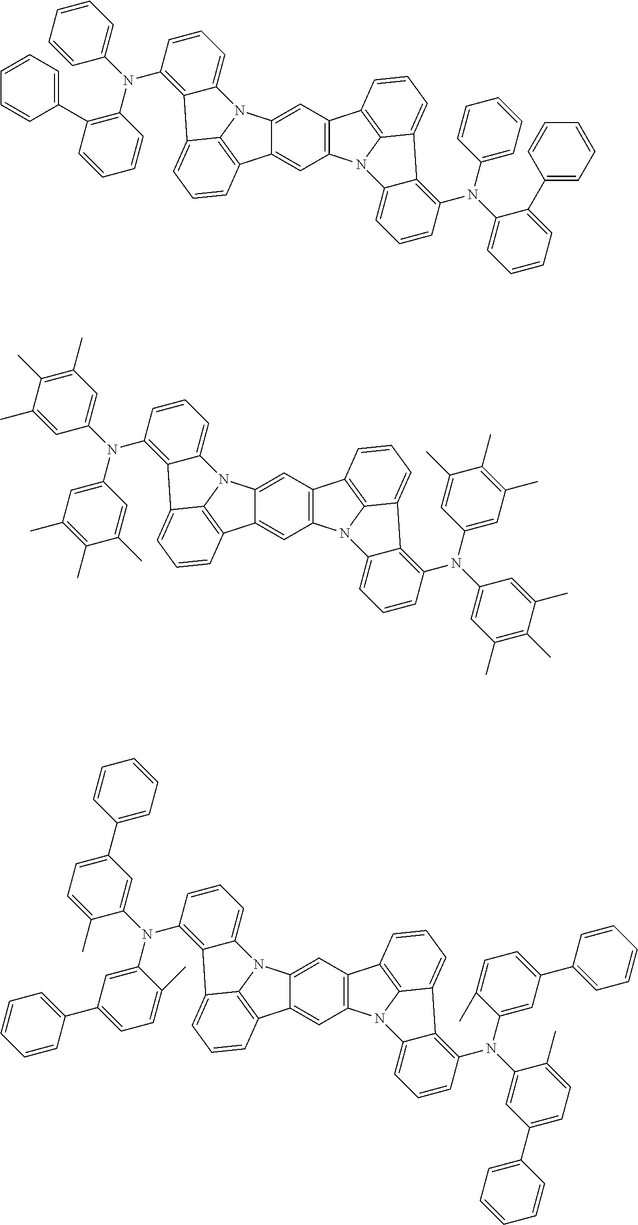

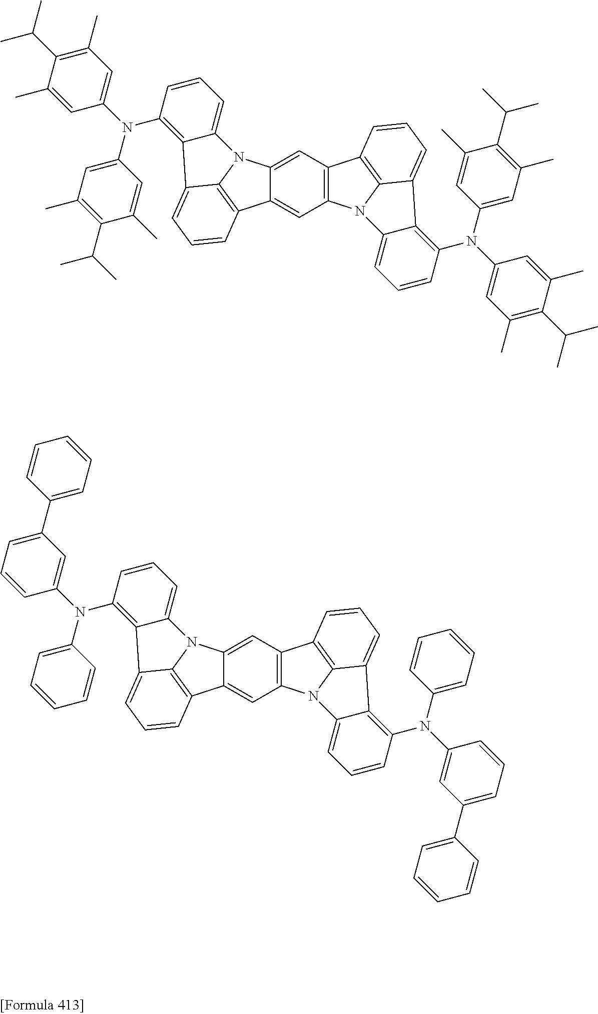 [SMILES: Cc1cc(N(c2cc(C)c(C(C)C)c(C)c2)c2cccc3c2c2cccc4c5cc6c(cc5n3c42)c2cccc3c4c(N(c5cc(C)c(C(C)C)c(C)c5)c5cc(C)c(C(C)C)c(C)c5)cccc4n6c23)cc(C)c1C(C)C ; c1ccc(-c2cccc(N(c3ccccc3)c3cccc4c3c3cccc5c6cc7c(cc6n4c53)c3cccc4c5c(N(c6ccccc6)c6cccc(-c8ccccc8)c6)cccc5n7c34)c2)cc1]

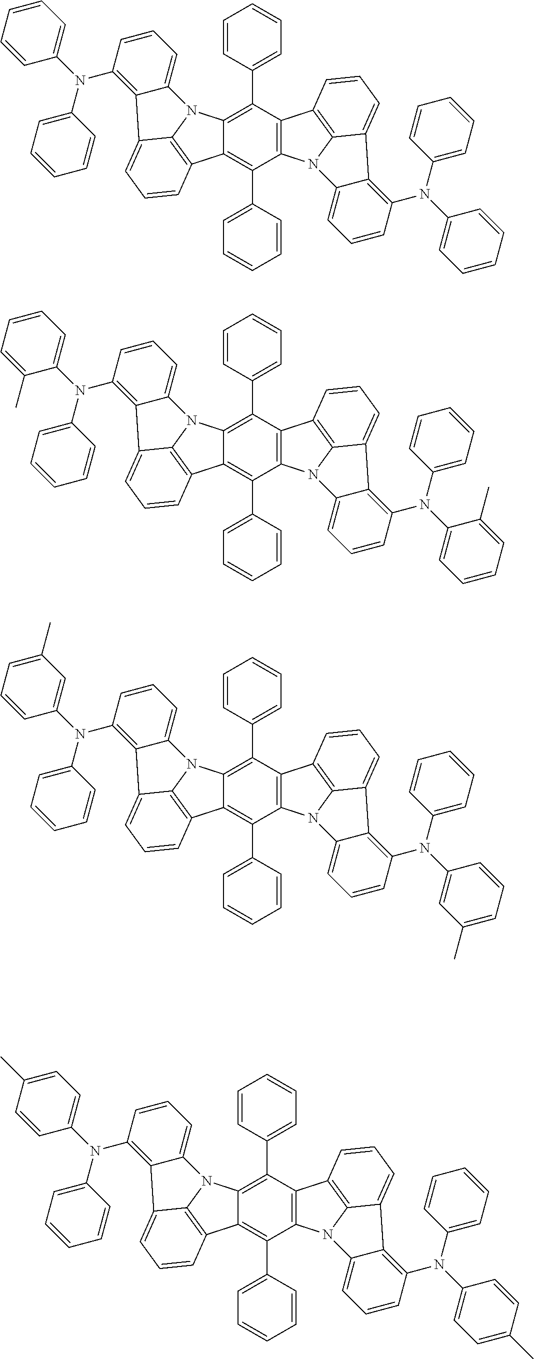

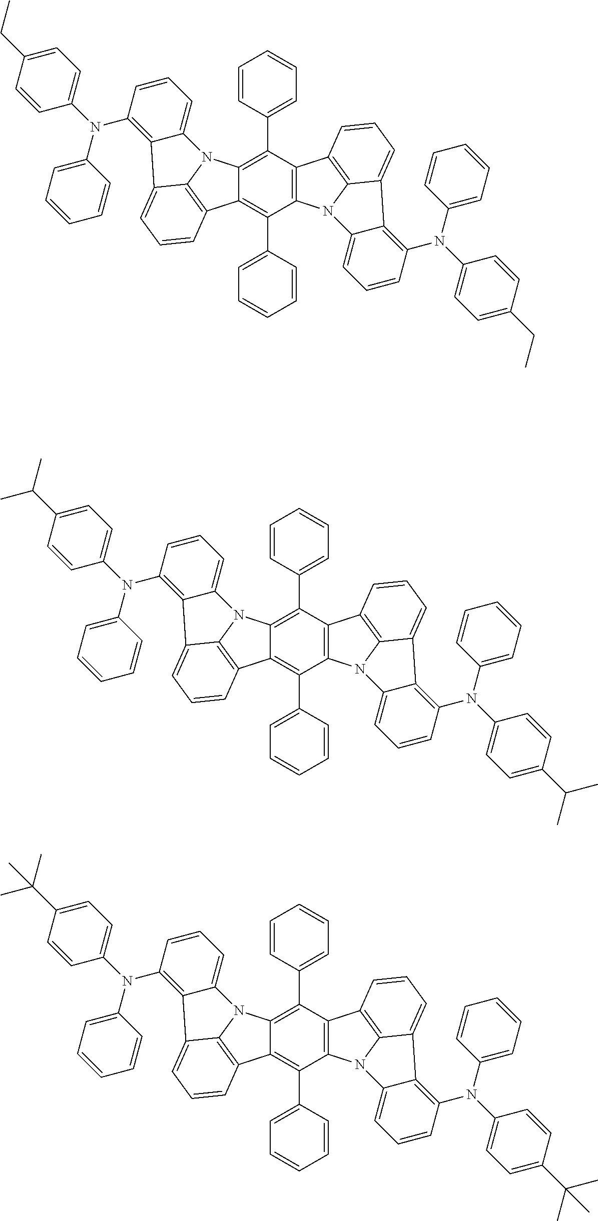

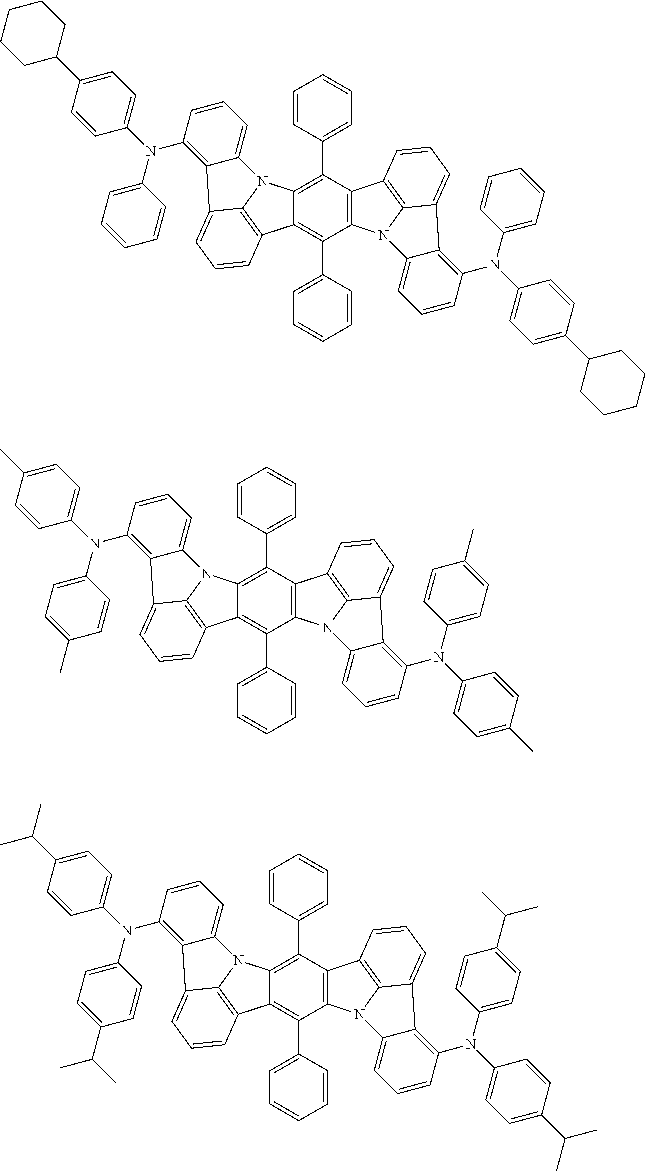

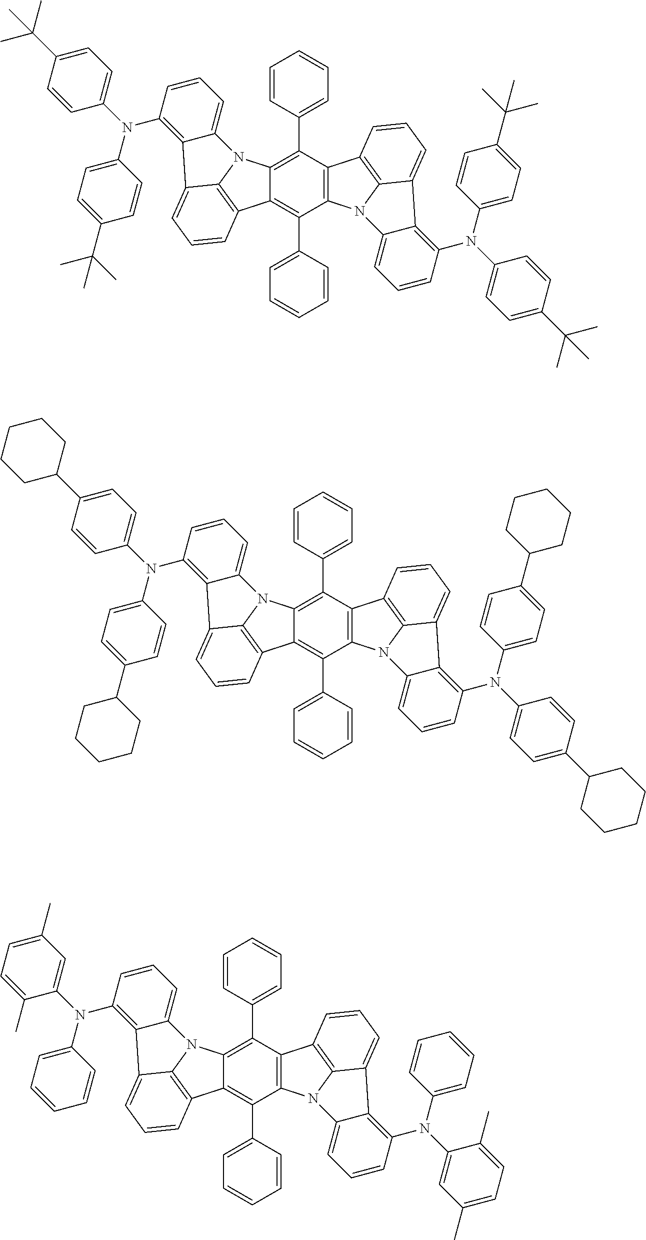 [SMILES: CC(C)(C)c1ccc(N(c2ccc(C(C)(C)C)cc2)c2cccc3c2c2cccc4c5c(-c6ccccc6)c6c(c(-c7ccccc7)c5n3c24)c2cccc3c4c(N(c5ccc(C(C)(C)C)cc5)c5ccc(C(C)(C)C)cc5)cccc4n6c32)cc1 ; c1ccc(-c2c3c4cccc5c6c(N(c7ccc(C8CCCCC8)cc7)c7ccc(C8CCCCC8)cc7)cccc6n(c3c(-c3ccccc3)c3c6cccc7c8c(N(c9ccc(C%10CCCCC%10)cc9)c9ccc(C%10CCCCC%10)cc9)cccc8n(c23)c76)c54)cc1 ; Cc1ccc(C)c(N(c2ccccc2)c2cccc3c2c2cccc4c5c(-c6ccccc6)c6c(c(-c7ccccc7)c5n3c24)c2cccc3c4c(N(c5ccccc5)c5cc(C)ccc5C)cccc4n6c32)c1]

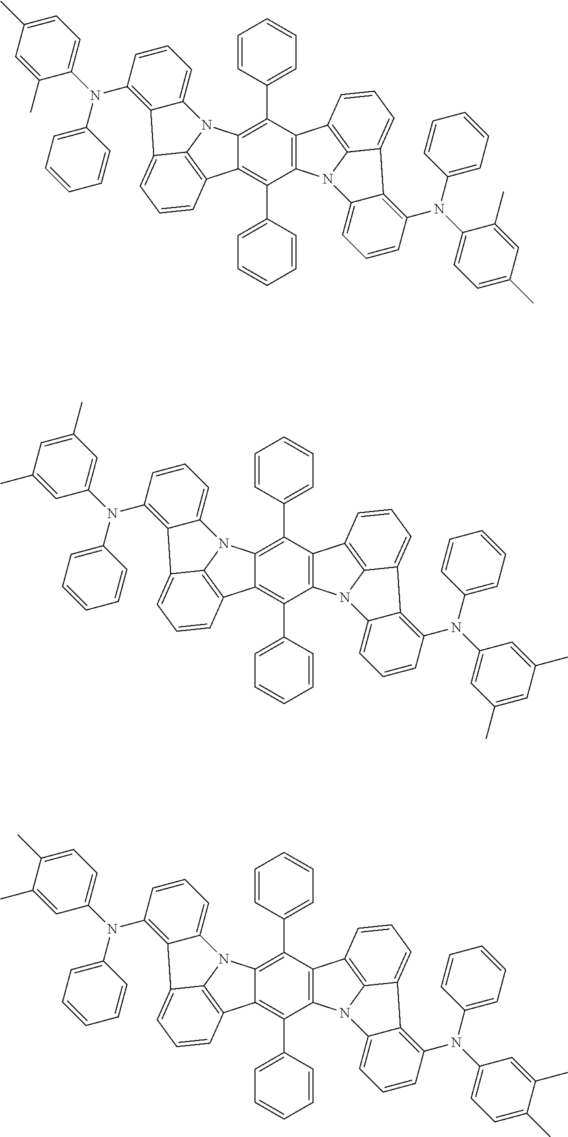

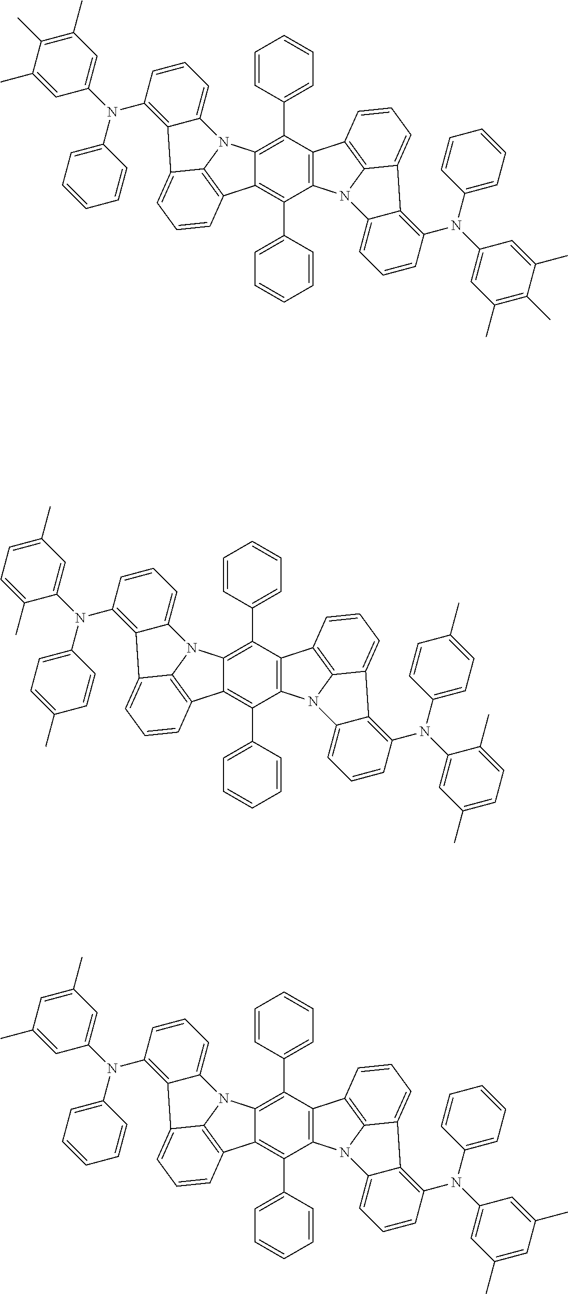

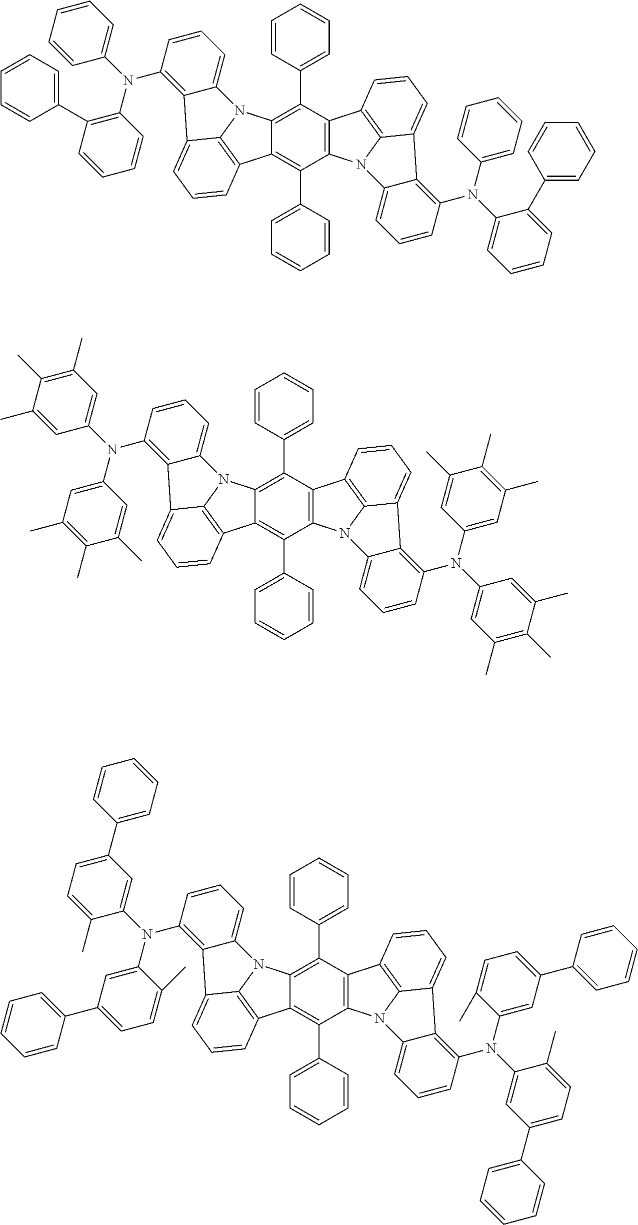

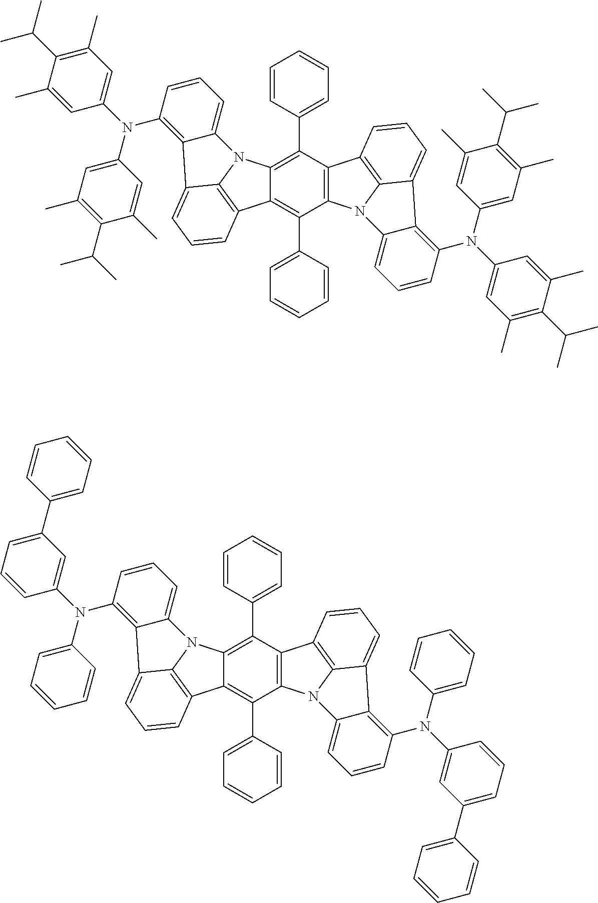 [SMILES: Cc1cc(N(c2cc(C)c(C(C)C)c(C)c2)c2cccc3c2c2cccc4c5c(-c6ccccc6)c6c(c(-c7ccccc7)c5n3c24)c2cccc3c4c(N(c5cc(C)c(C(C)C)c(C)c5)c5cc(C)c(C(C)C)c(C)c5)cccc4n6c32)cc(C)c1C(C)C ; c1ccc(-c2cccc(N(c3ccccc3)c3cccc4c3c3cccc5c6c(-c7ccccc7)c7c(c(-c8ccccc8)c6n4c35)c3cccc4c5c(N(c6ccccc6)c6cccc(-c8ccccc8)c6)cccc5n7c43)c2)cc1]

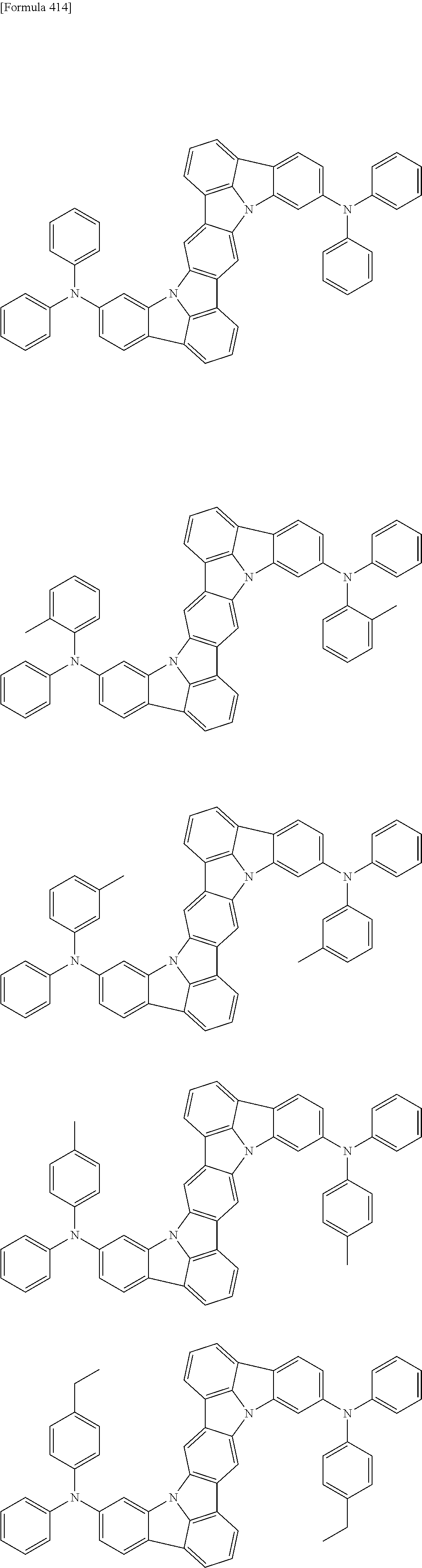

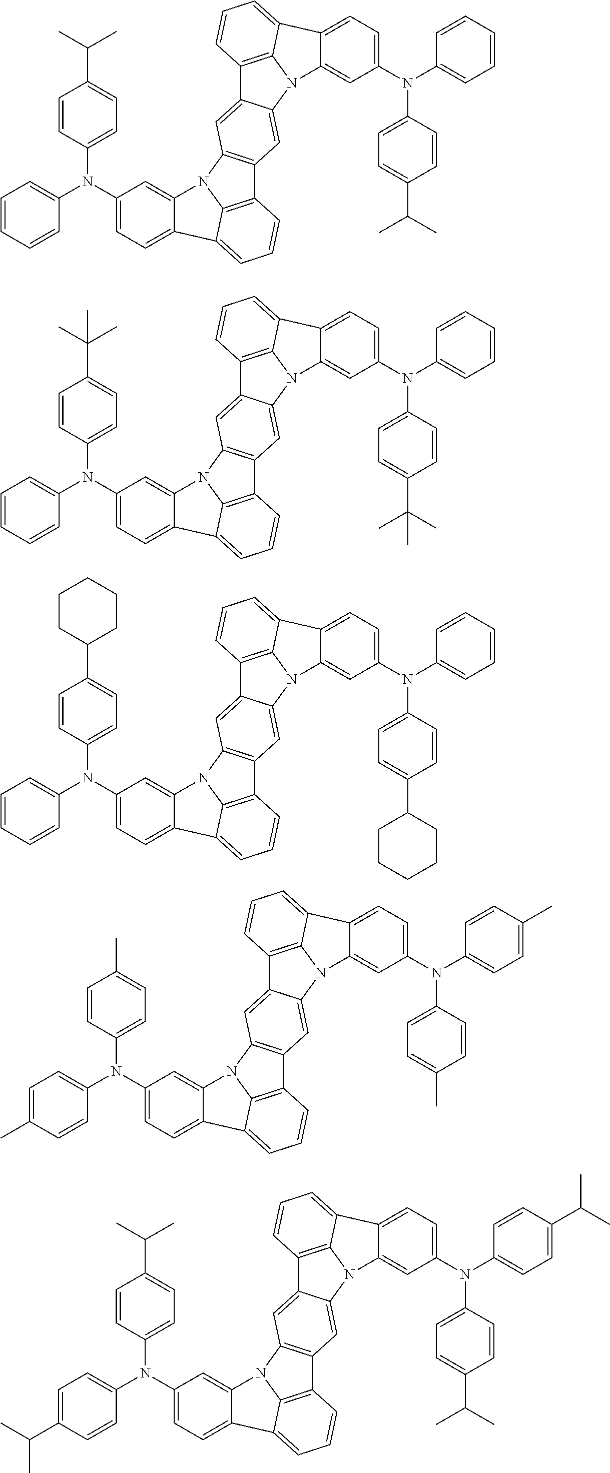

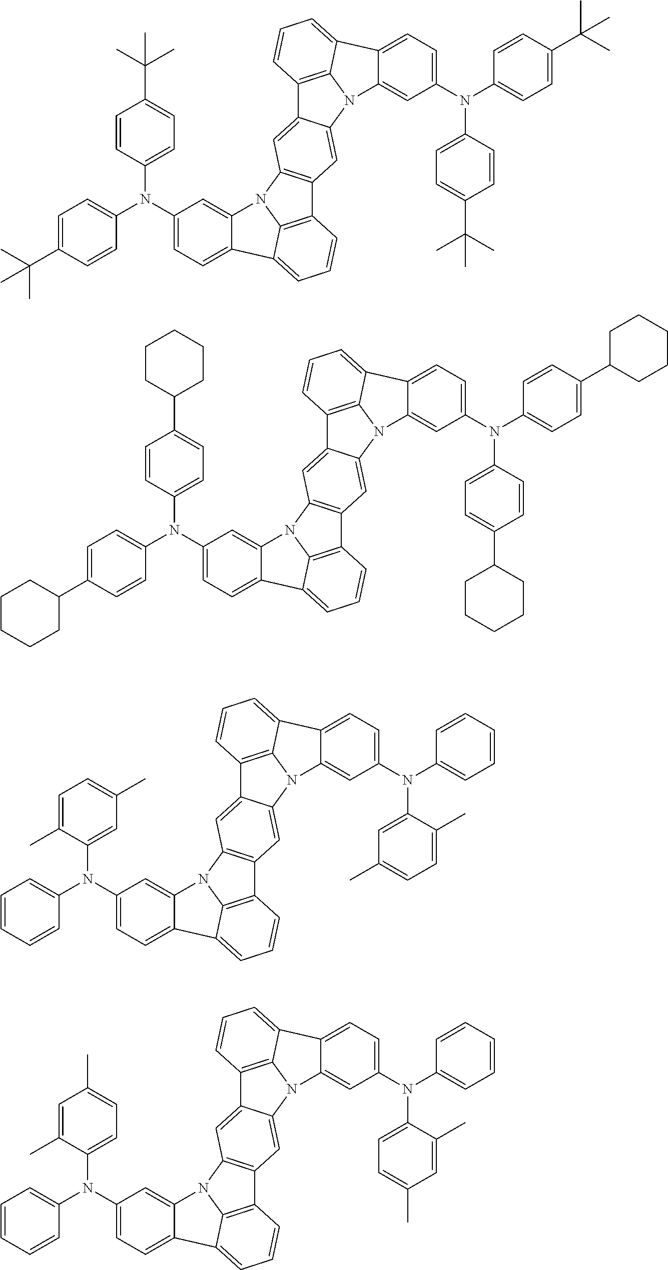 [SMILES: CC(C)(C)c1ccc(N(c2ccc(C(C)(C)C)cc2)c2ccc3c4cccc5c6cc7c(cc6n(c3c2)c45)c2cccc3c4ccc(N(c5ccc(C(C)(C)C)cc5)c5ccc(C(C)(C)C)cc5)cc4n7c32)cc1 ; c1cc2c3ccc(N(c4ccc(C5CCCCC5)cc4)c4ccc(C5CCCCC5)cc4)cc3n3c4cc5c6cccc7c8ccc(N(c9ccc(C%10CCCCC%10)cc9)c9ccc(C%10CCCCC%10)cc9)cc8n(c5cc4c(c1)c23)c76 ; Cc1ccc(C)c(N(c2ccccc2)c2ccc3c4cccc5c6cc7c(cc6n(c3c2)c45)c2cccc3c4ccc(N(c5ccccc5)c5cc(C)ccc5C)cc4n7c32)c1 ; Cc1ccc(N(c2ccccc2)c2ccc3c4cccc5c6cc7c(cc6n(c3c2)c45)c2cccc3c4ccc(N(c5ccccc5)c5ccc(C)cc5C)cc4n7c32)c(C)c1]

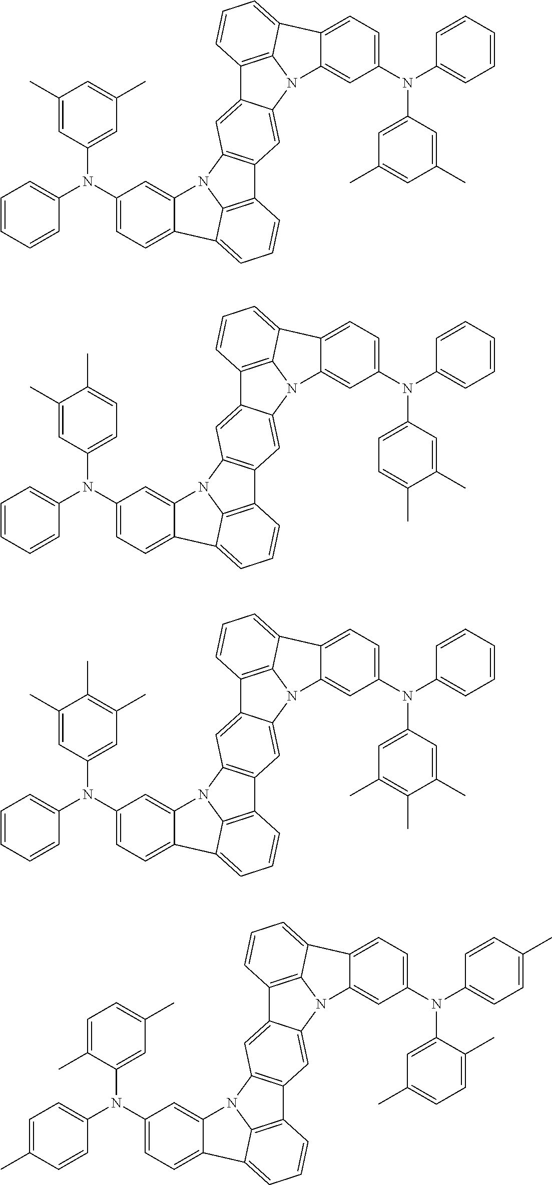

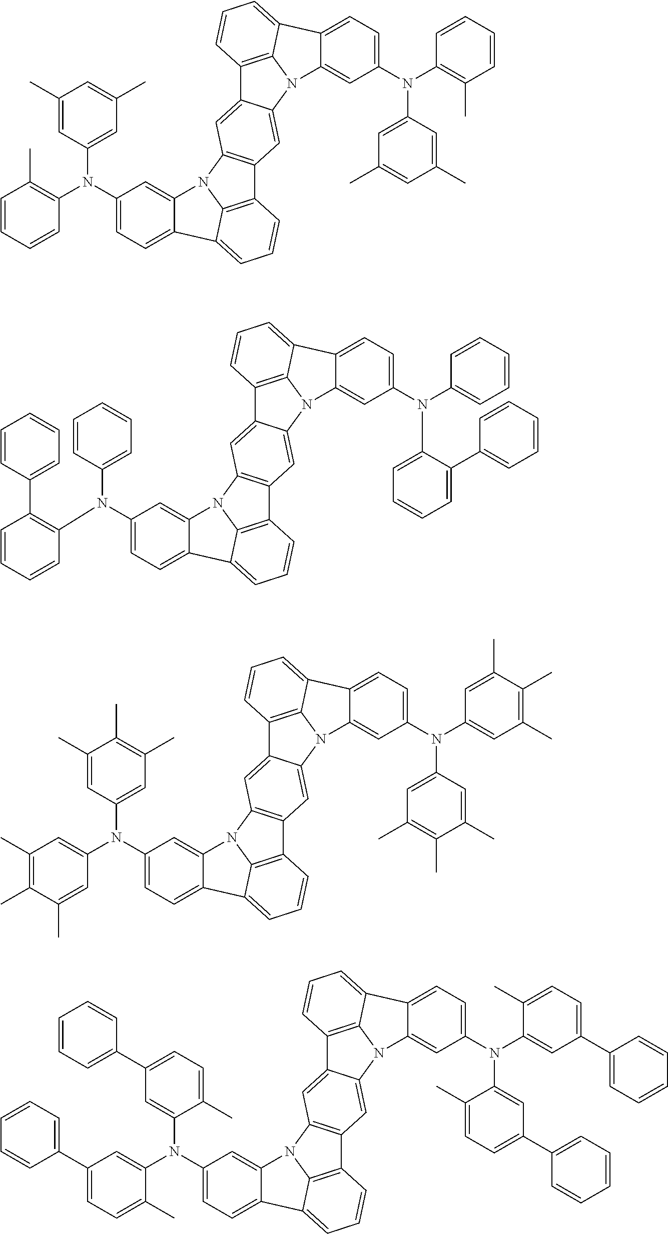

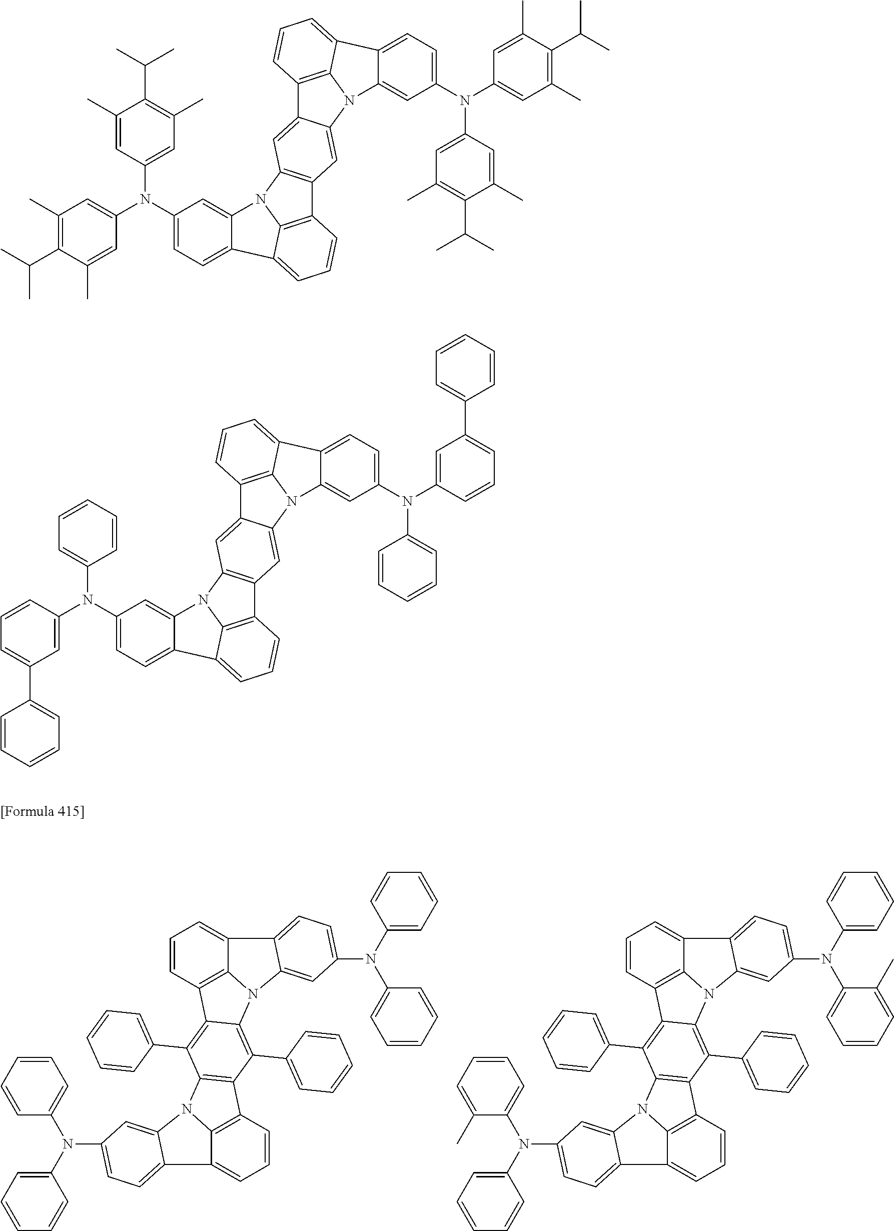

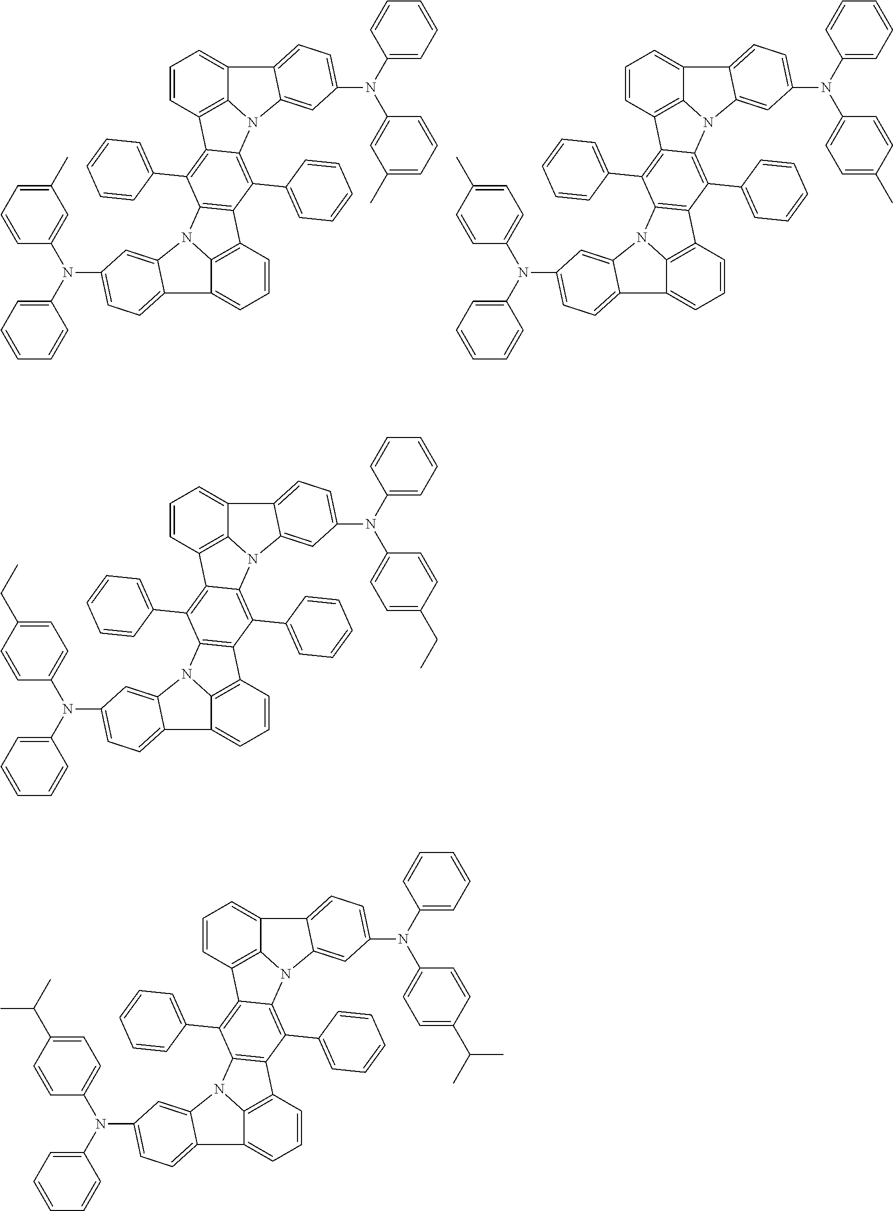

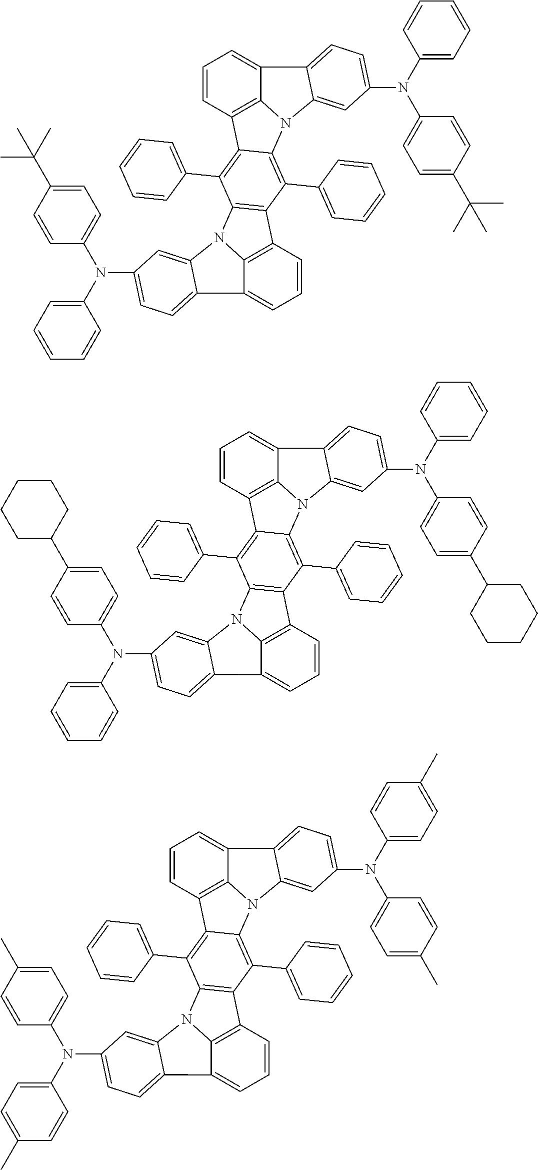 [SMILES: CC(C)(C)c1ccc(N(c2ccccc2)c2ccc3c4cccc5c6c(-c7ccccc7)c7c(c(-c8ccccc8)c6n(c3c2)c45)c2cccc3c4ccc(N(c5ccccc5)c5ccc(C(C)(C)C)cc5)cc4n7c32)cc1 ; c1ccc(-c2c3c4cccc5c6ccc(N(c7ccccc7)c7ccc(C8CCCCC8)cc7)cc6n(c3c(-c3ccccc3)c3c6cccc7c8ccc(N(c9ccccc9)c9ccc(C%10CCCCC%10)cc9)cc8n(c23)c76)c54)cc1 ; Cc1ccc(N(c2ccc(C)cc2)c2ccc3c4cccc5c6c(-c7ccccc7)c7c(c(-c8ccccc8)c6n(c3c2)c45)c2cccc3c4ccc(N(c5ccc(C)cc5)c5ccc(C)cc5)cc4n7c32)cc1]

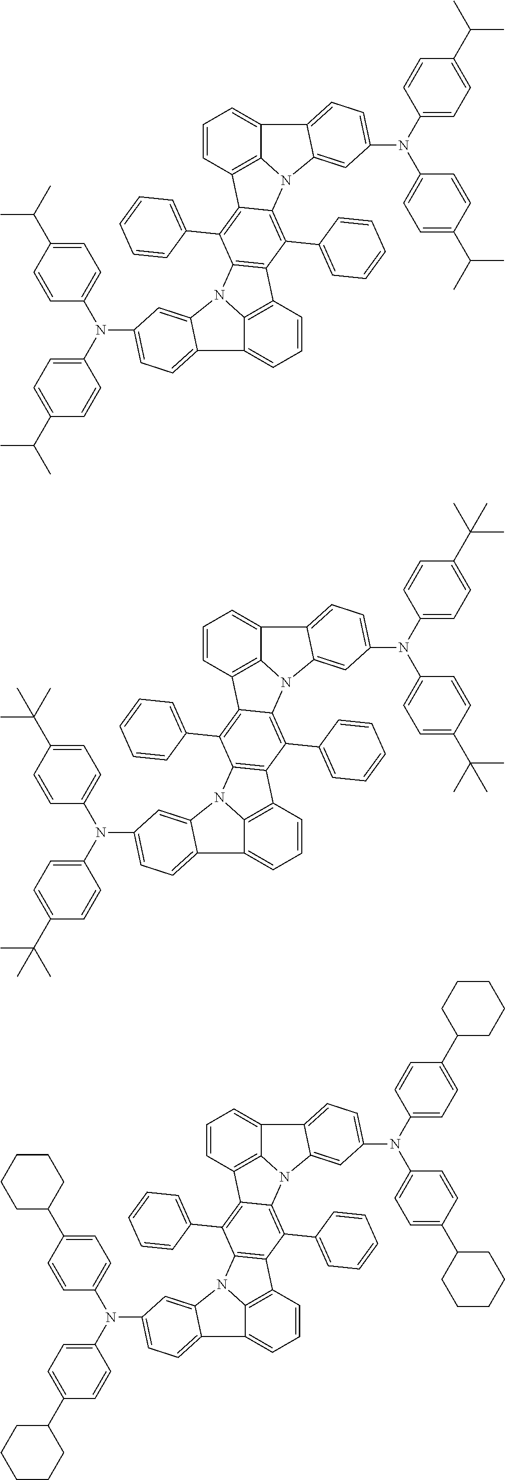

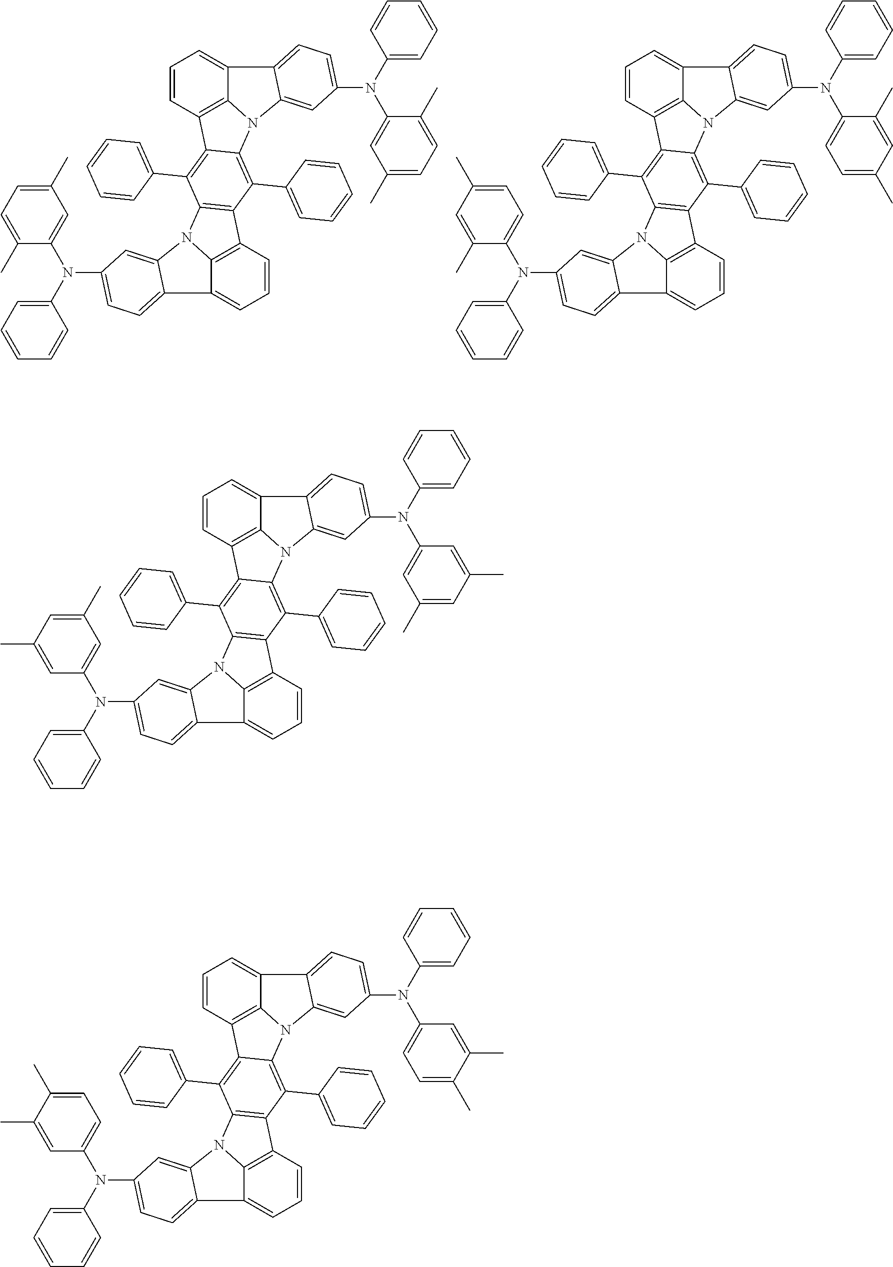

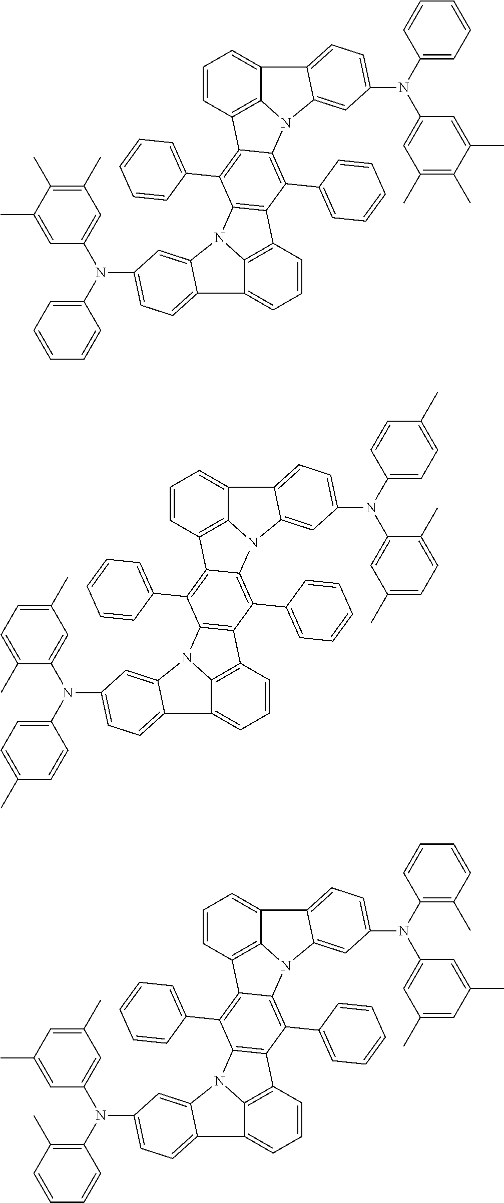

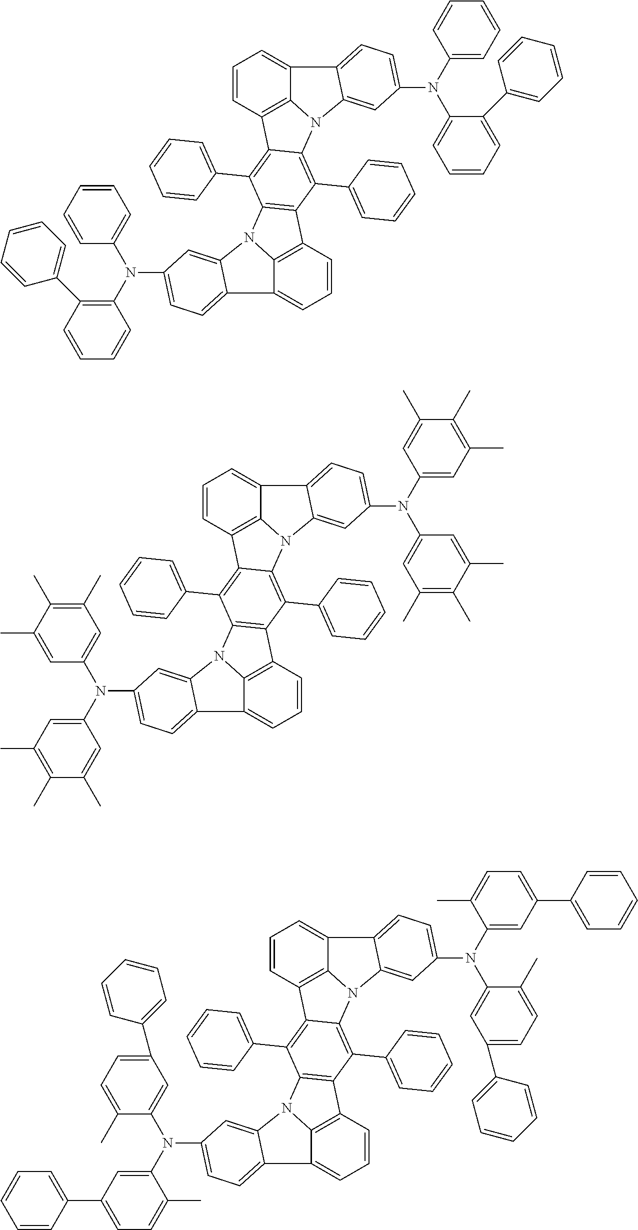

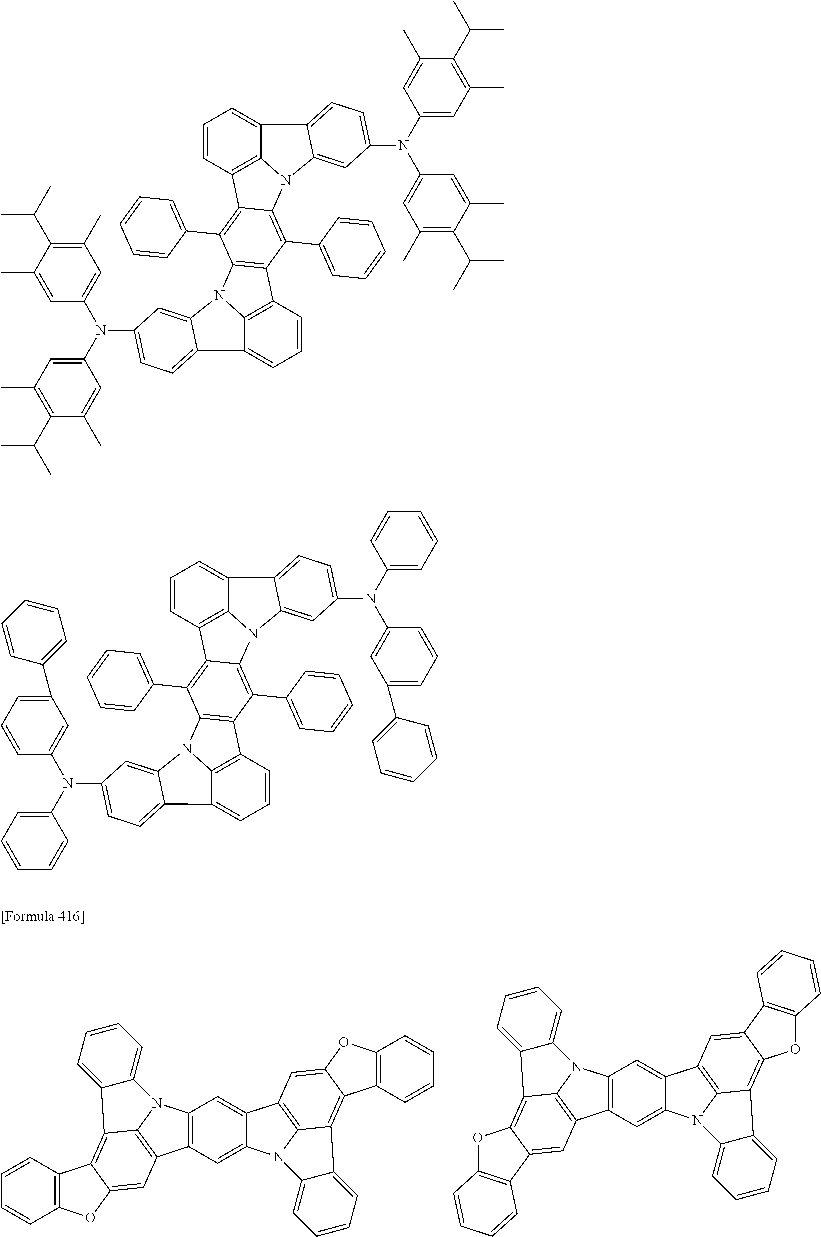 [SMILES: Cc1cc(N(c2cc(C)c(C(C)C)c(C)c2)c2ccc3c4cccc5c6c(-c7ccccc7)c7c(c(-c8ccccc8)c6n(c3c2)c45)c2cccc3c4ccc(N(c5cc(C)c(C(C)C)c(C)c5)c5cc(C)c(C(C)C)c(C)c5)cc4n7c32)cc(C)c1C(C)C ; c1ccc(-c2cccc(N(c3ccccc3)c3ccc4c5cccc6c7c(-c8ccccc8)c8c(c(-c9ccccc9)c7n(c4c3)c56)c3cccc4c5ccc(N(c6ccccc6)c6cccc(-c7ccccc7)c6)cc5n8c43)c2)cc1 ; c1ccc2c(c1)oc1cc3c4cc5c(cc4n4c6ccccc6c(c12)c34)c1cc2oc3ccccc3c2c2c3ccccc3n5c12 ; c1ccc2c(c1)oc1c2cc2c3cc4c(cc3n3c5ccccc5c1c23)c1cc2c3ccccc3oc2c2c3ccccc3n4c12]

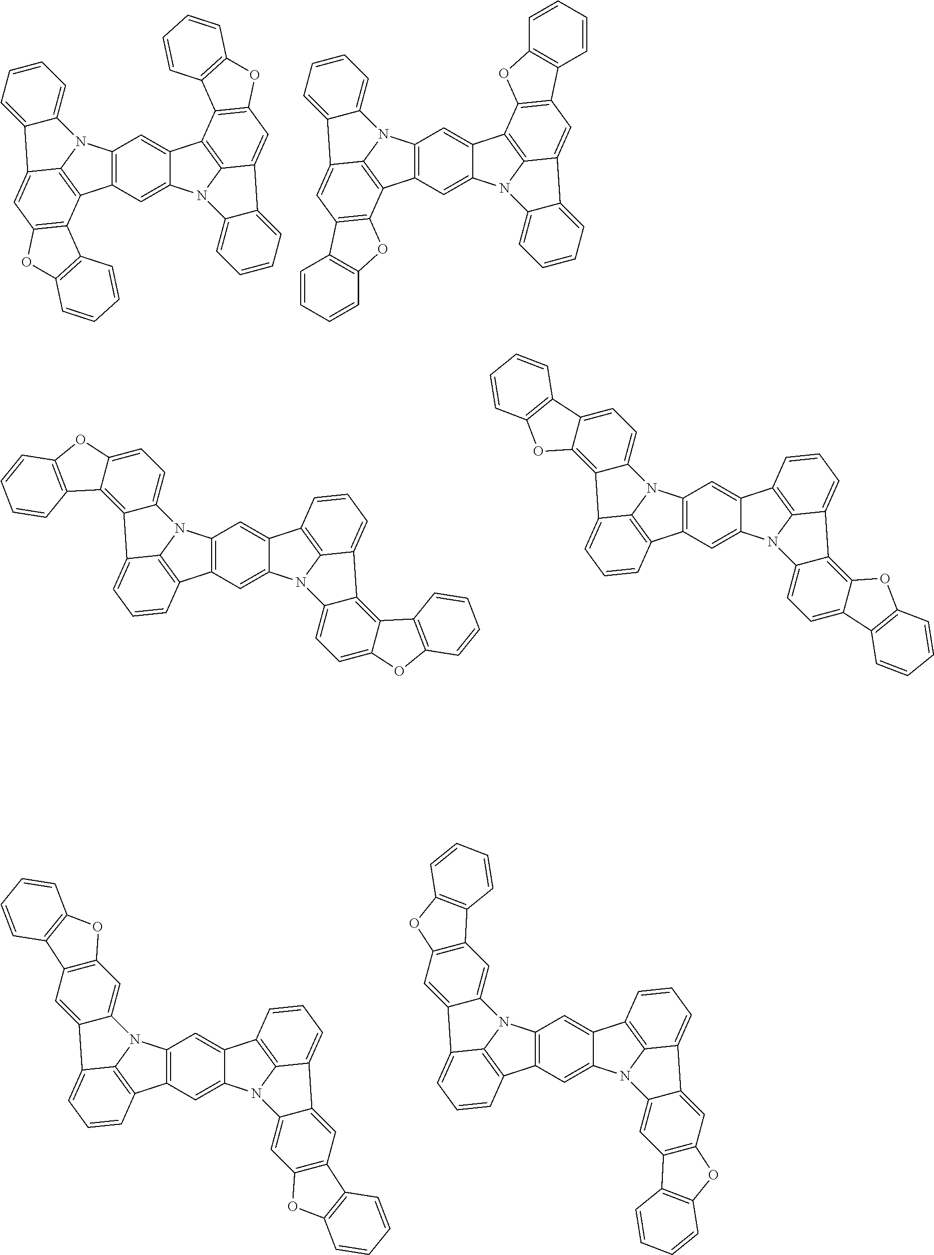

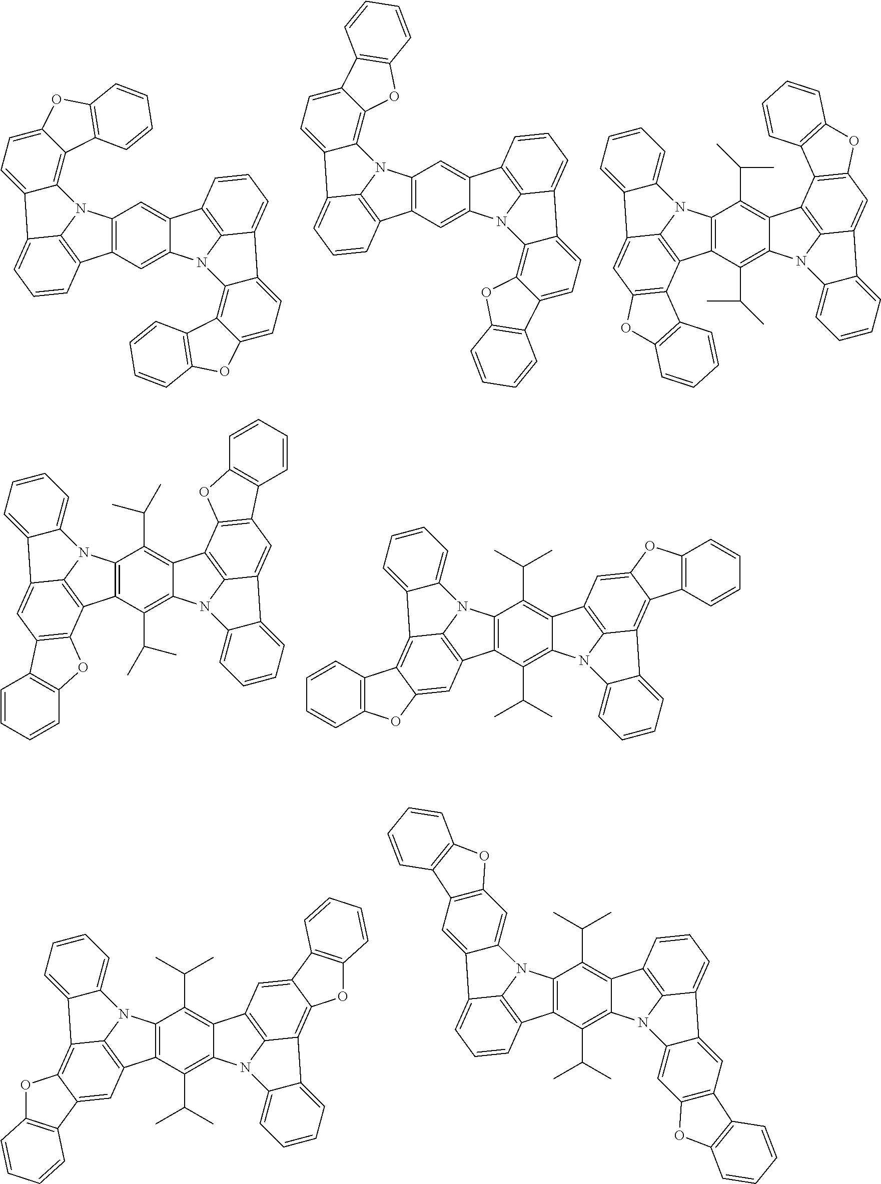

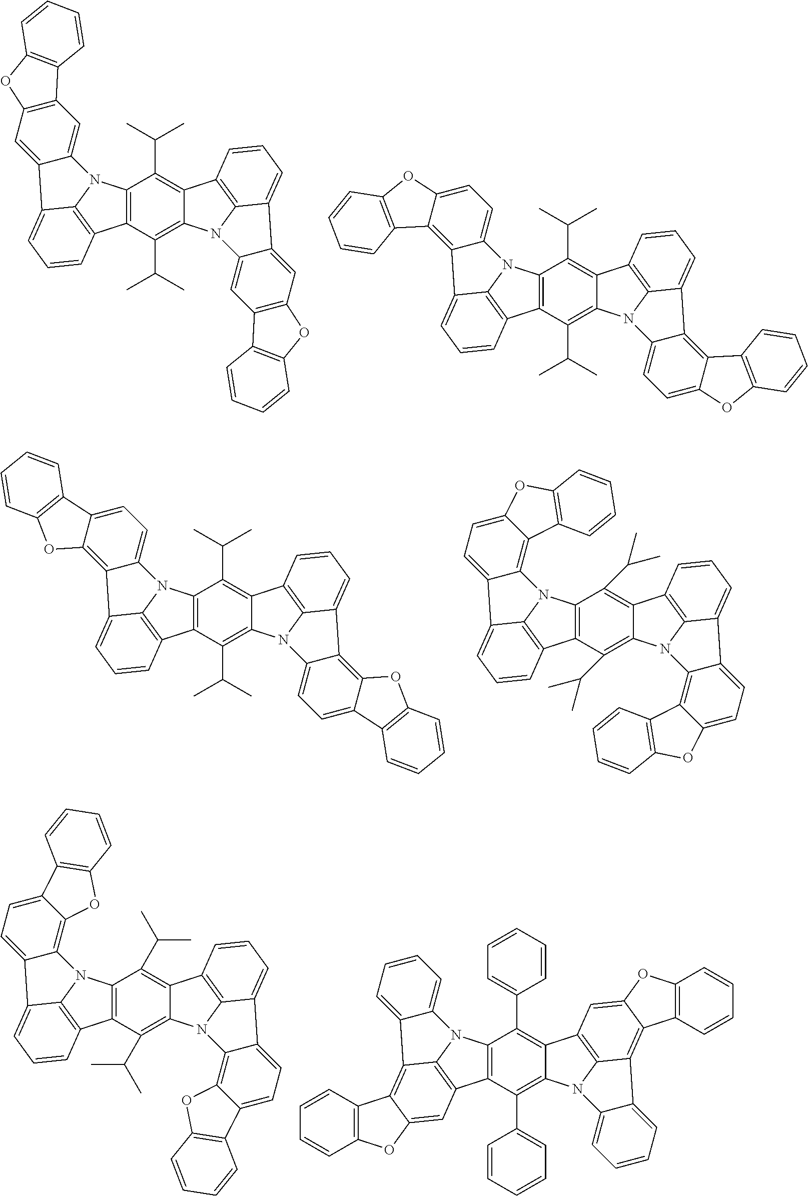

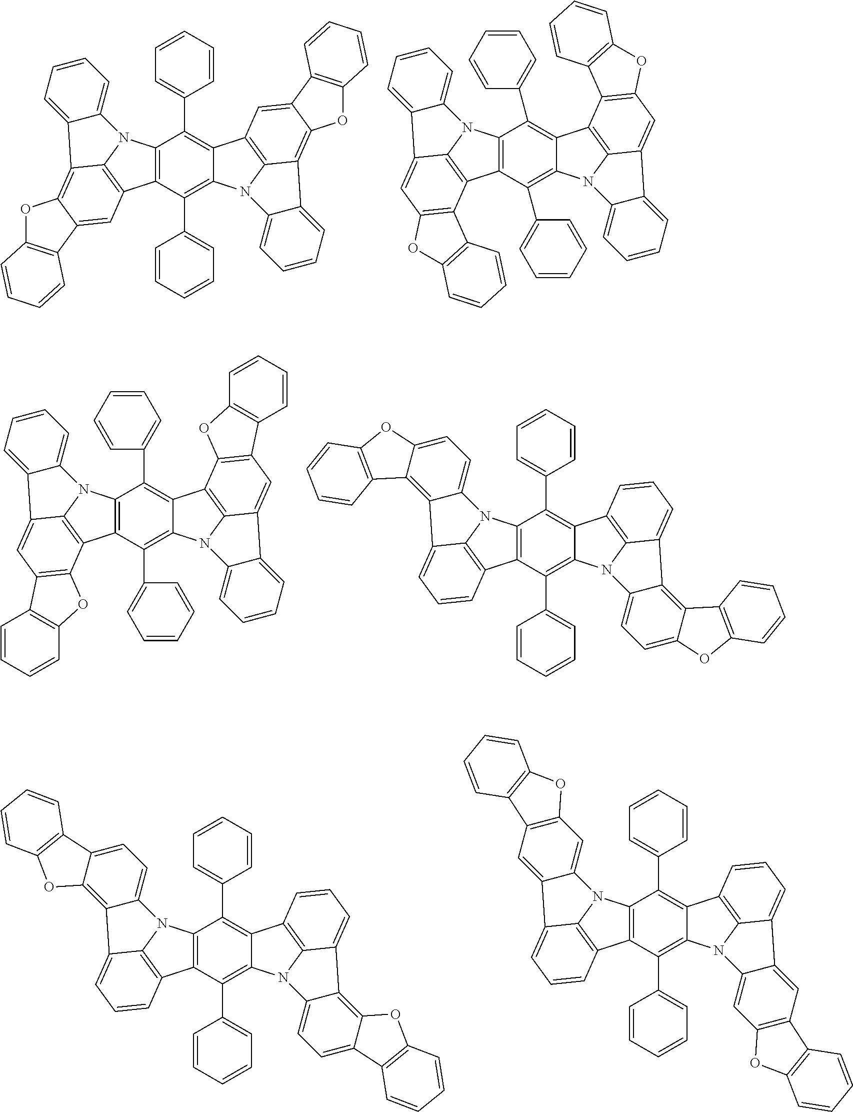

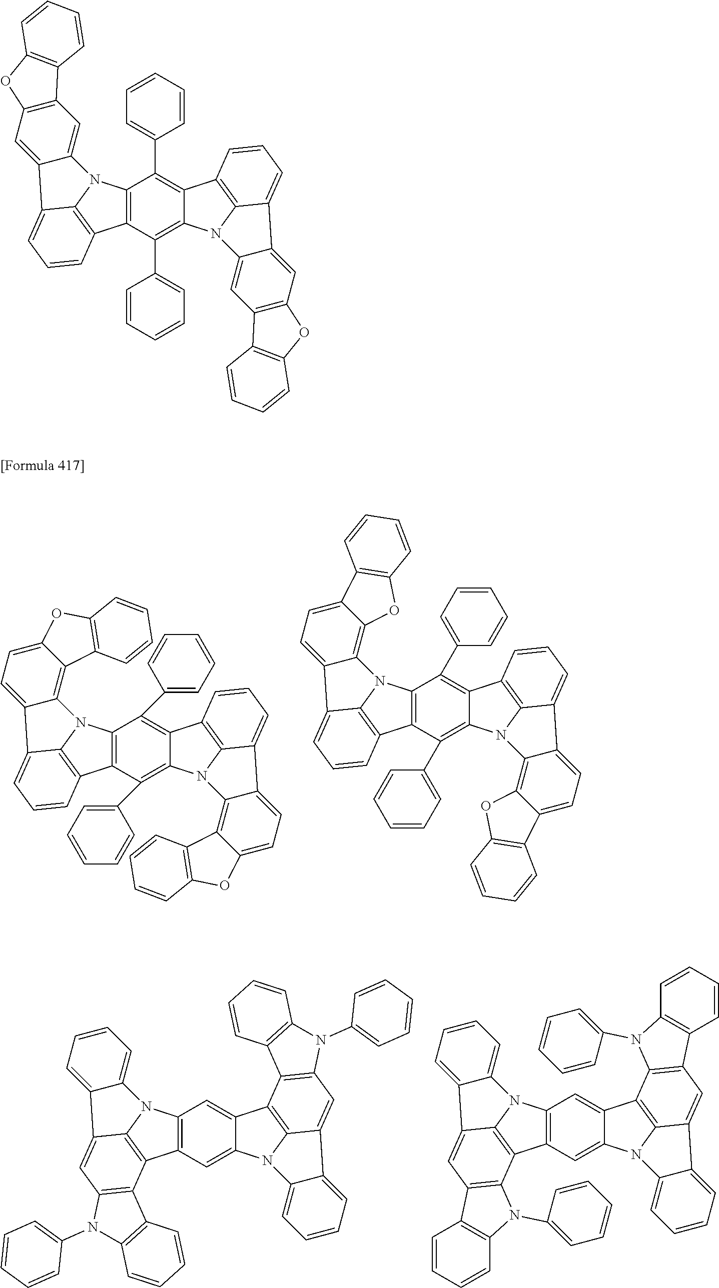

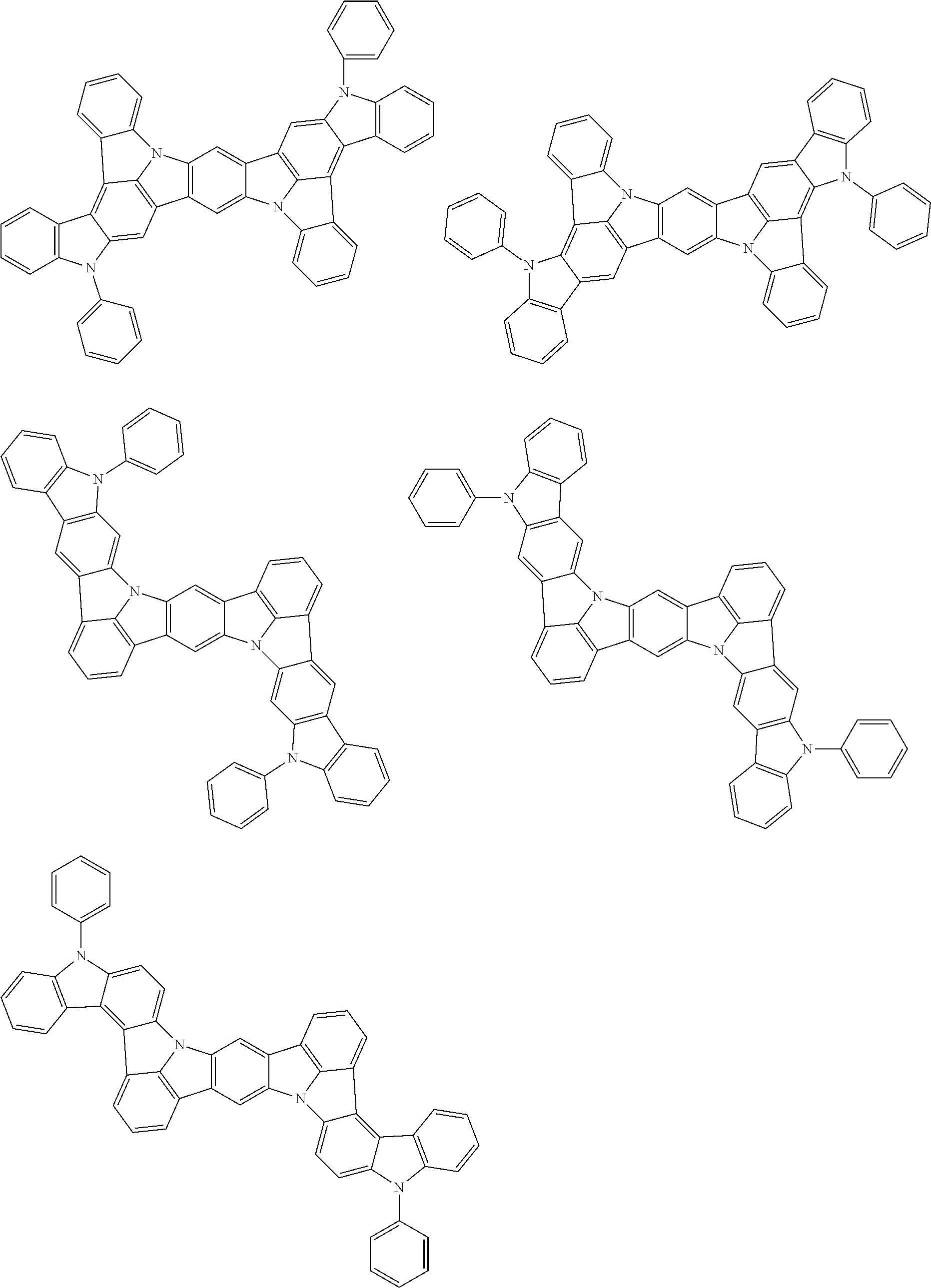

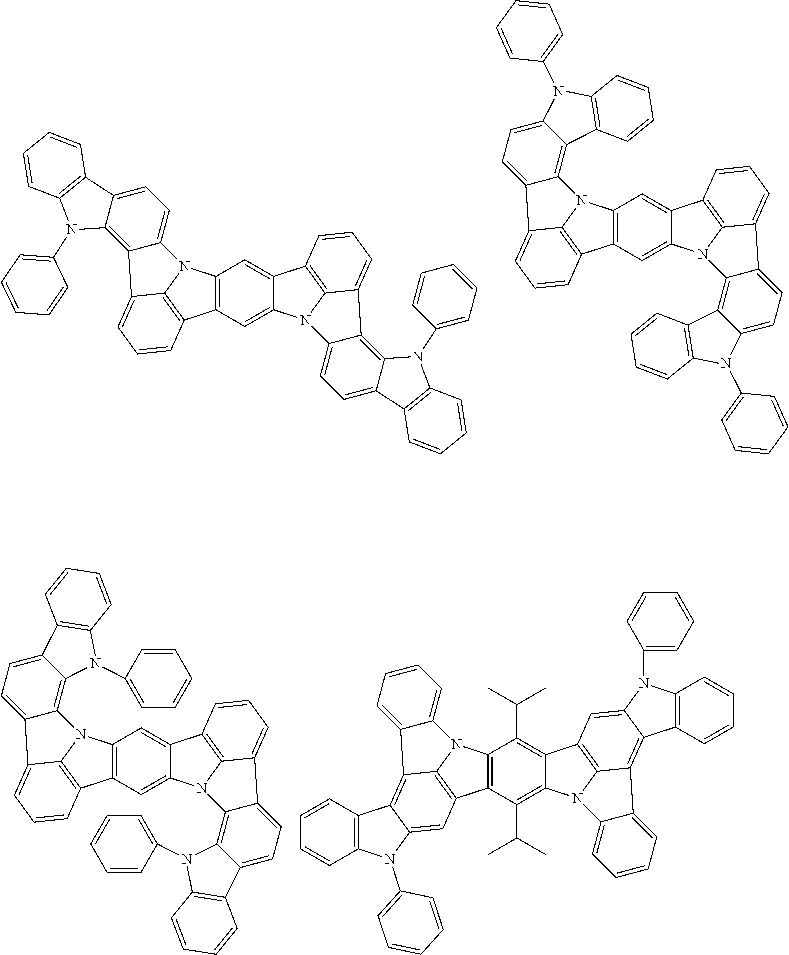

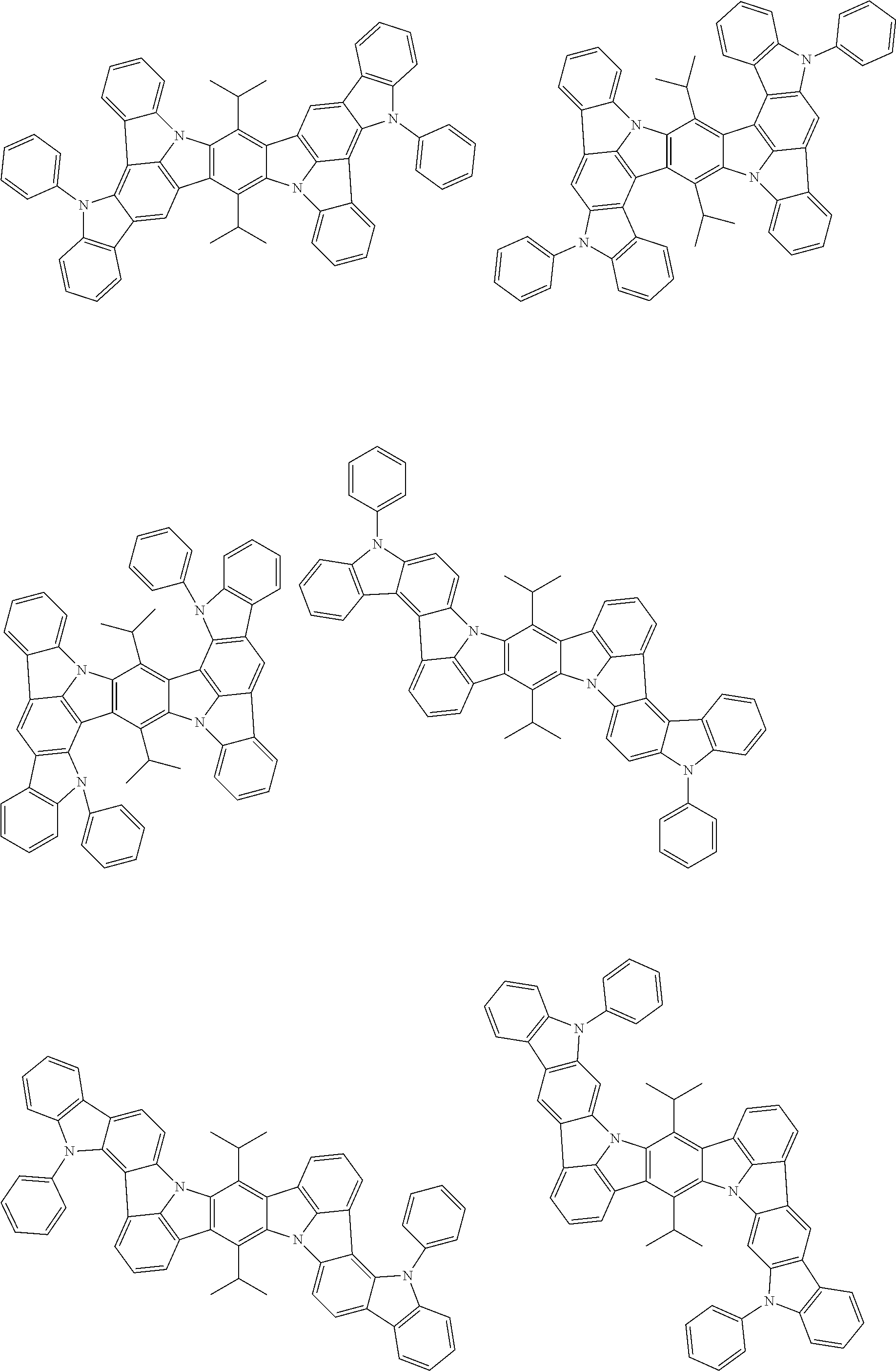

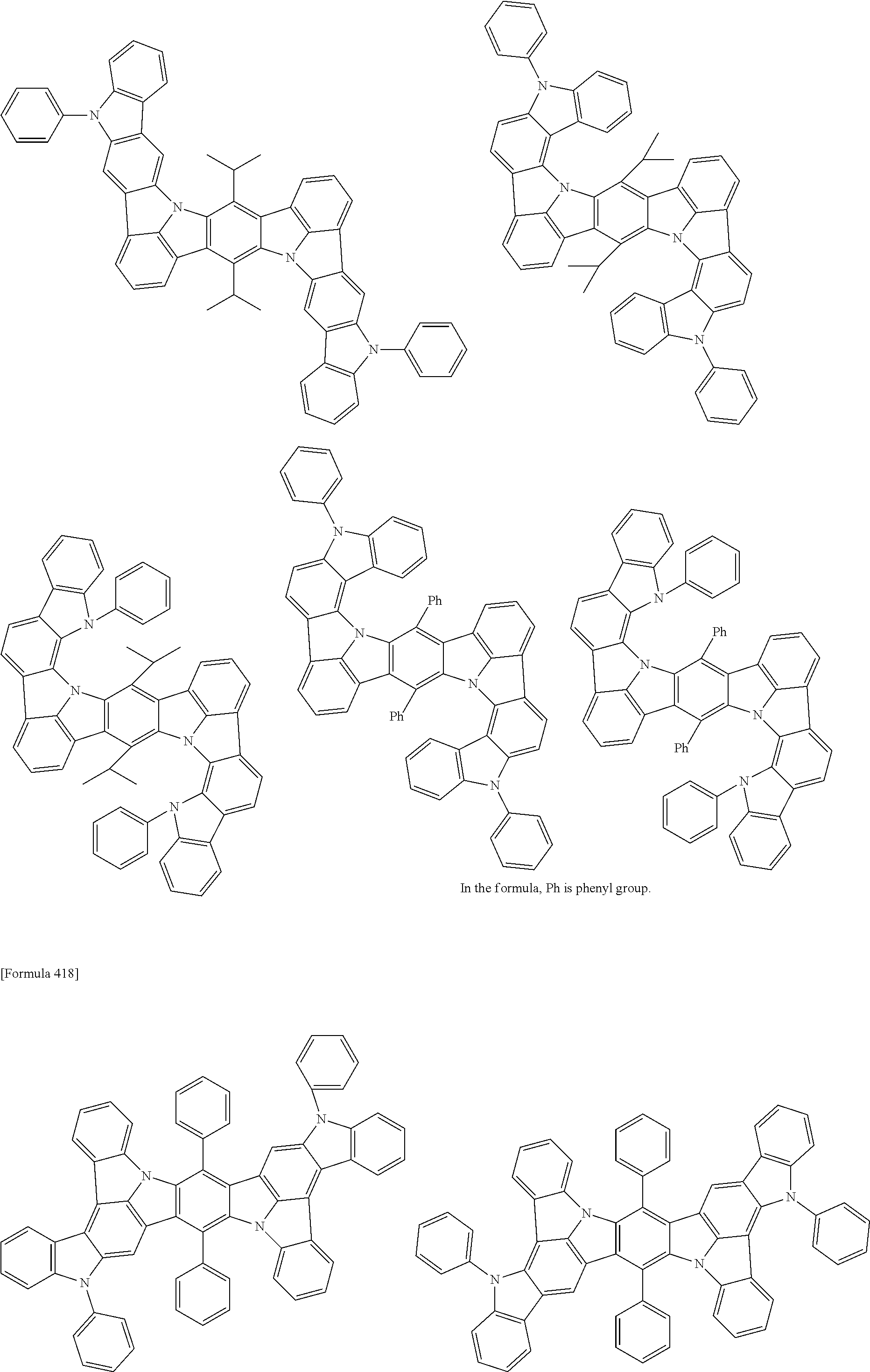

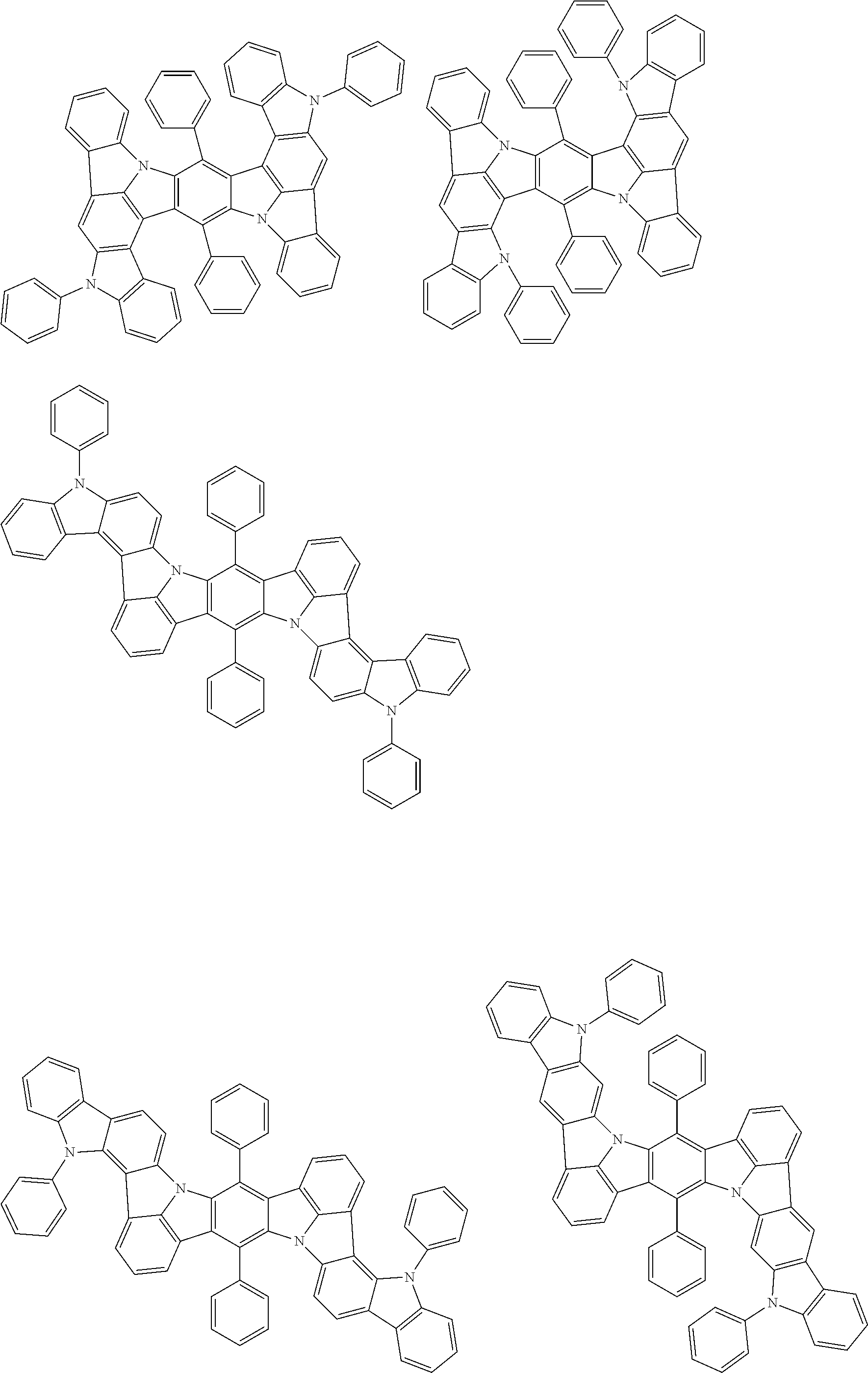

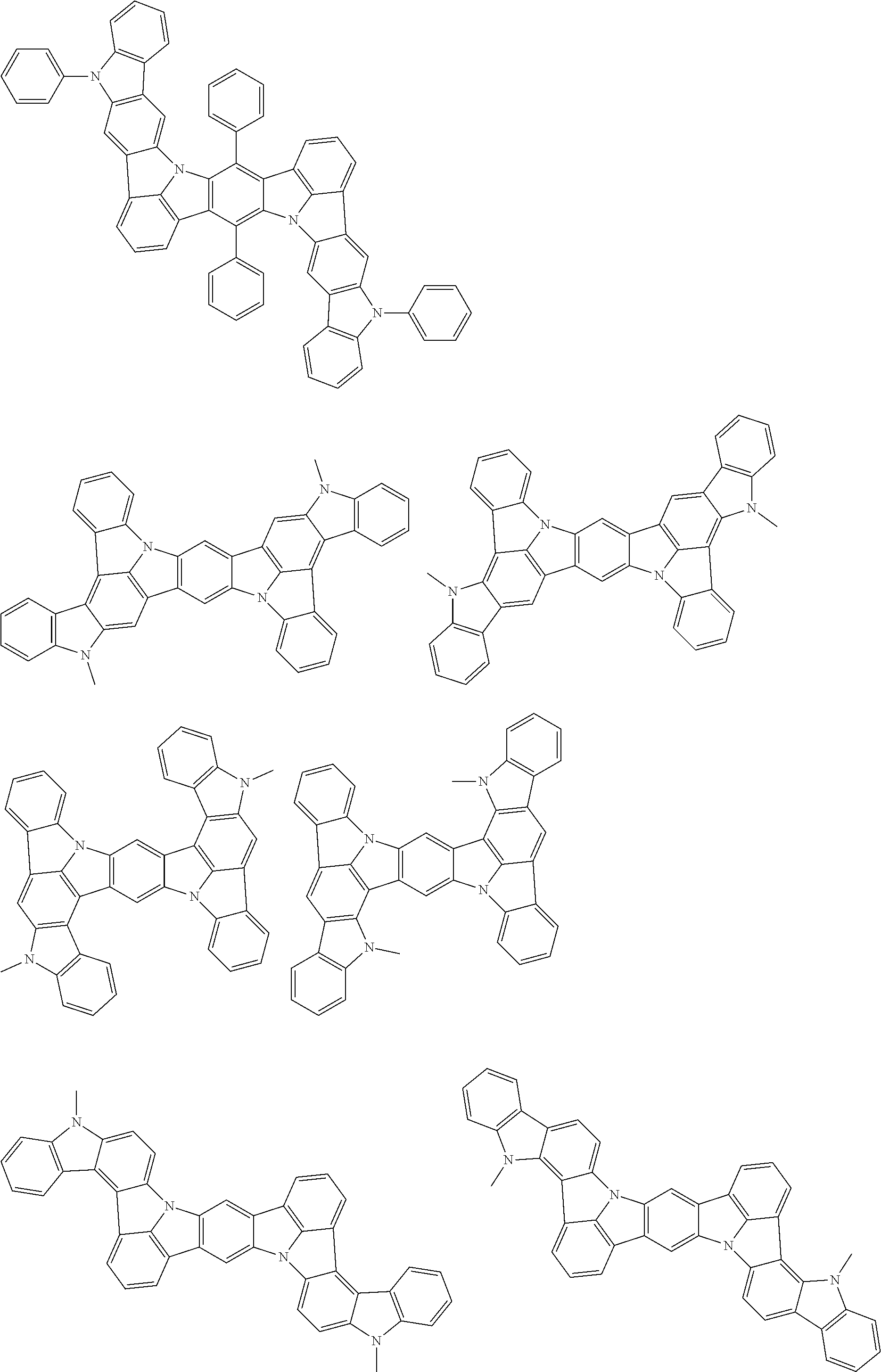

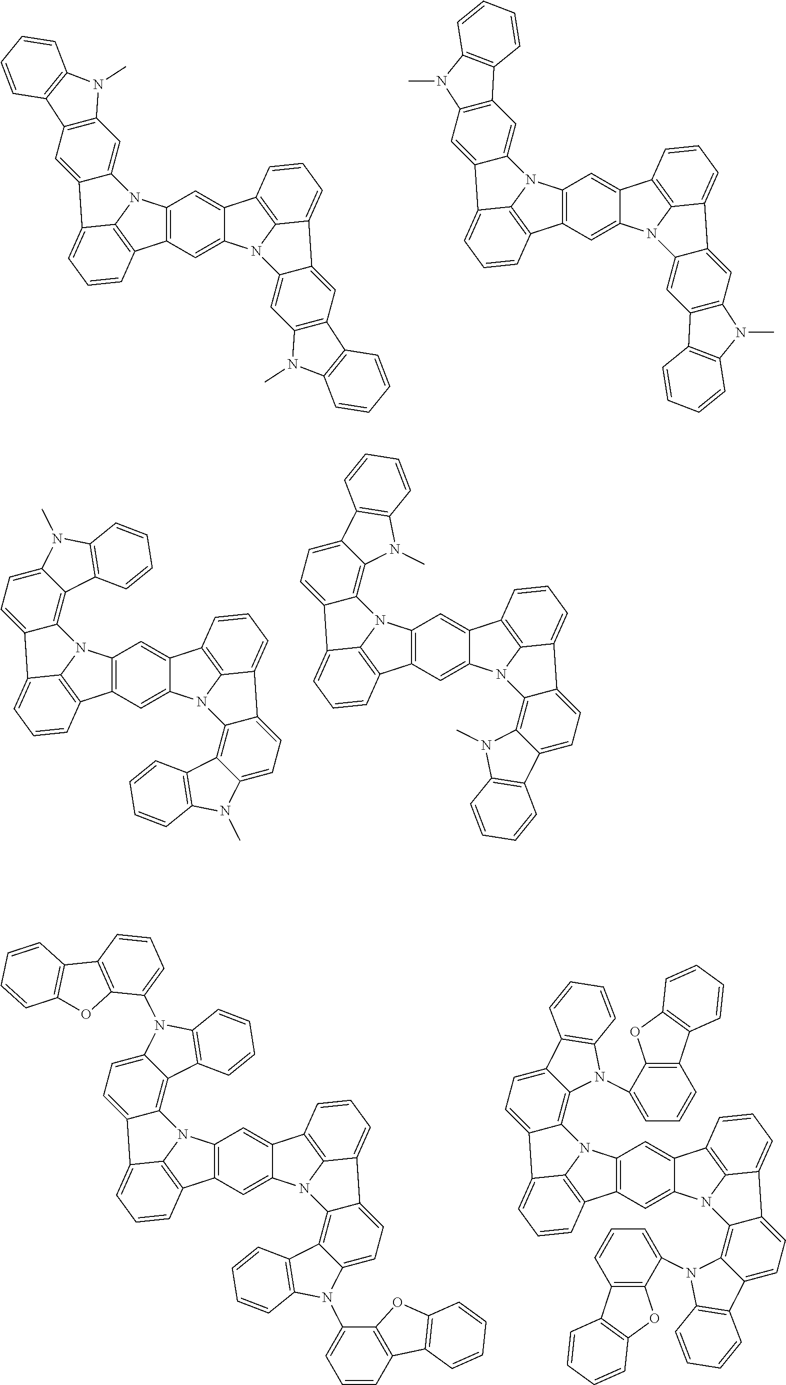

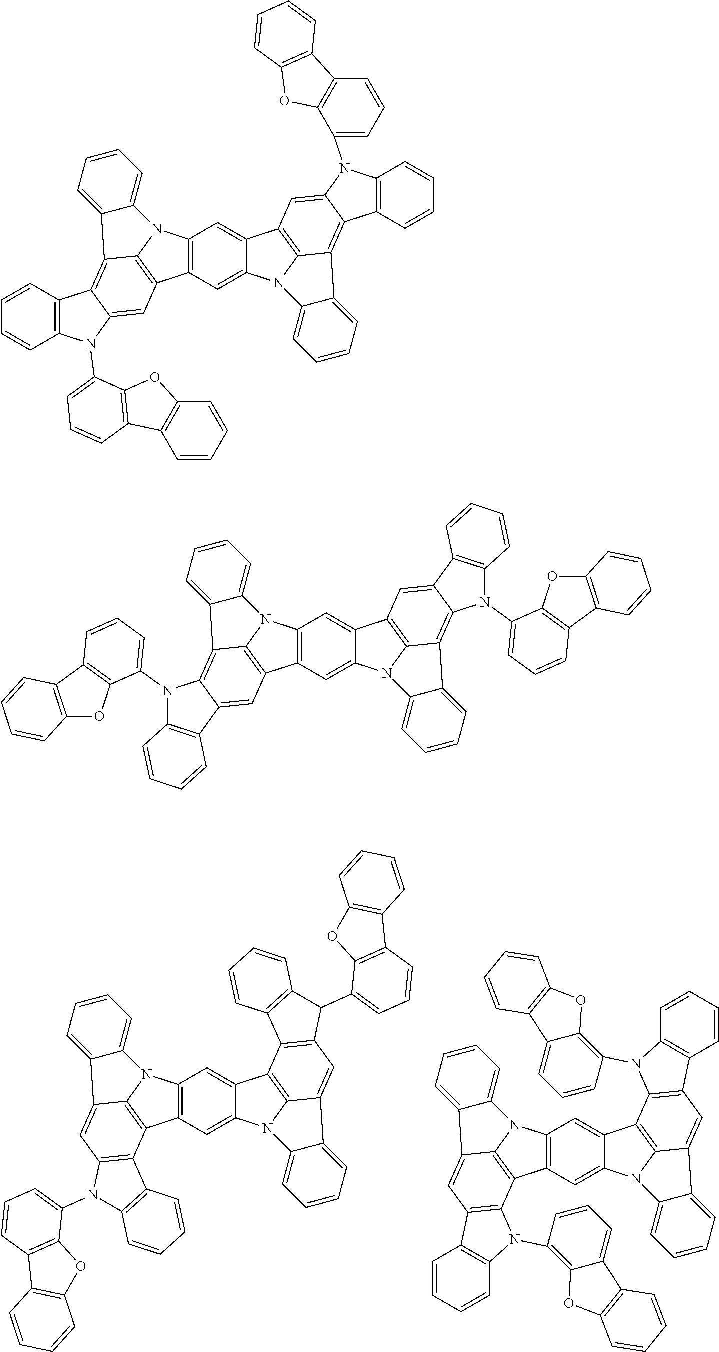

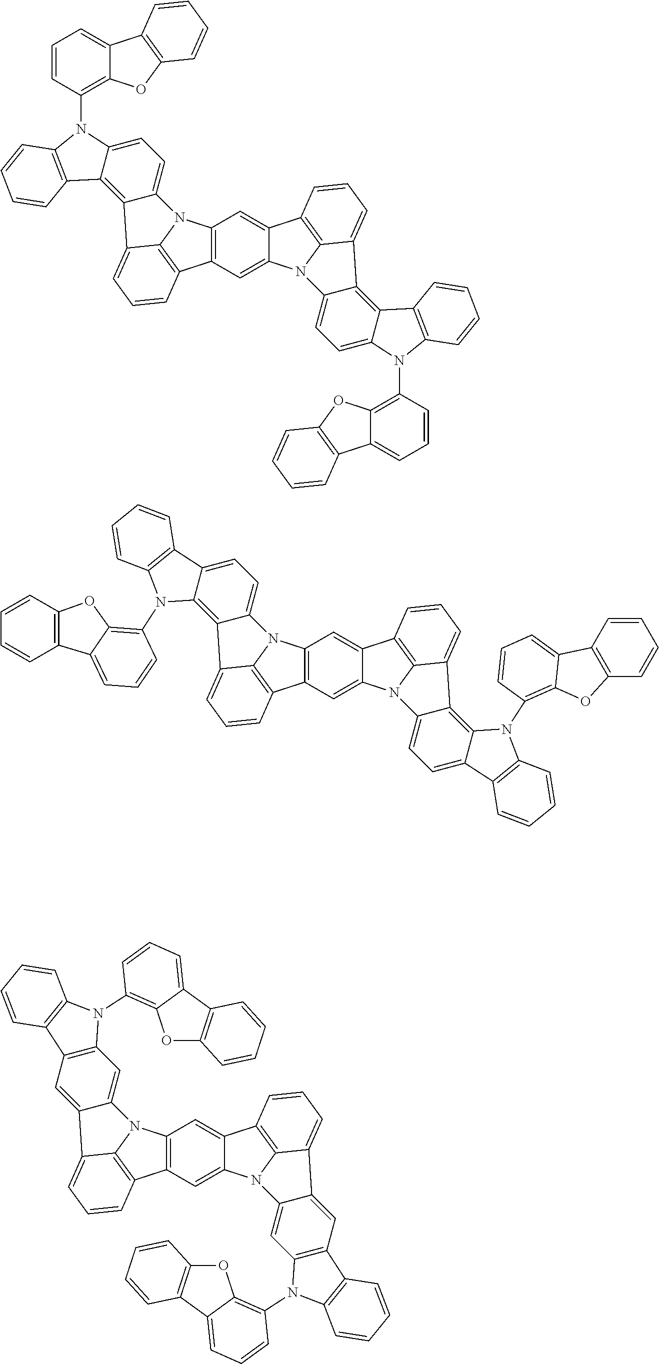 [SMILES: c1ccc2c(c1)oc1c(-n3c4ccccc4c4c5c6cccc7c8cc9c(cc8n(c5ccc43)c76)c3cccc4c5c6c7ccccc7n(-c7cccc8c7oc7ccccc78)c6ccc5n9c34)cccc12 ; c1ccc2c(c1)oc1c(-n3c4ccccc4c4ccc5c(c6cccc7c8cc9c(cc8n5c76)c5cccc6c7c8c(ccc7n9c56)c5ccccc5n8-c5cccc6c5oc5ccccc56)c43)cccc12 ; c1ccc2c(c1)oc1c(-n3c4ccccc4c4cc5c6cccc7c8cc9c(cc8n(c5cc43)c76)c3cccc4c5cc6c7ccccc7n(-c7cccc8c7oc7ccccc78)c6cc5n9c34)cccc12]

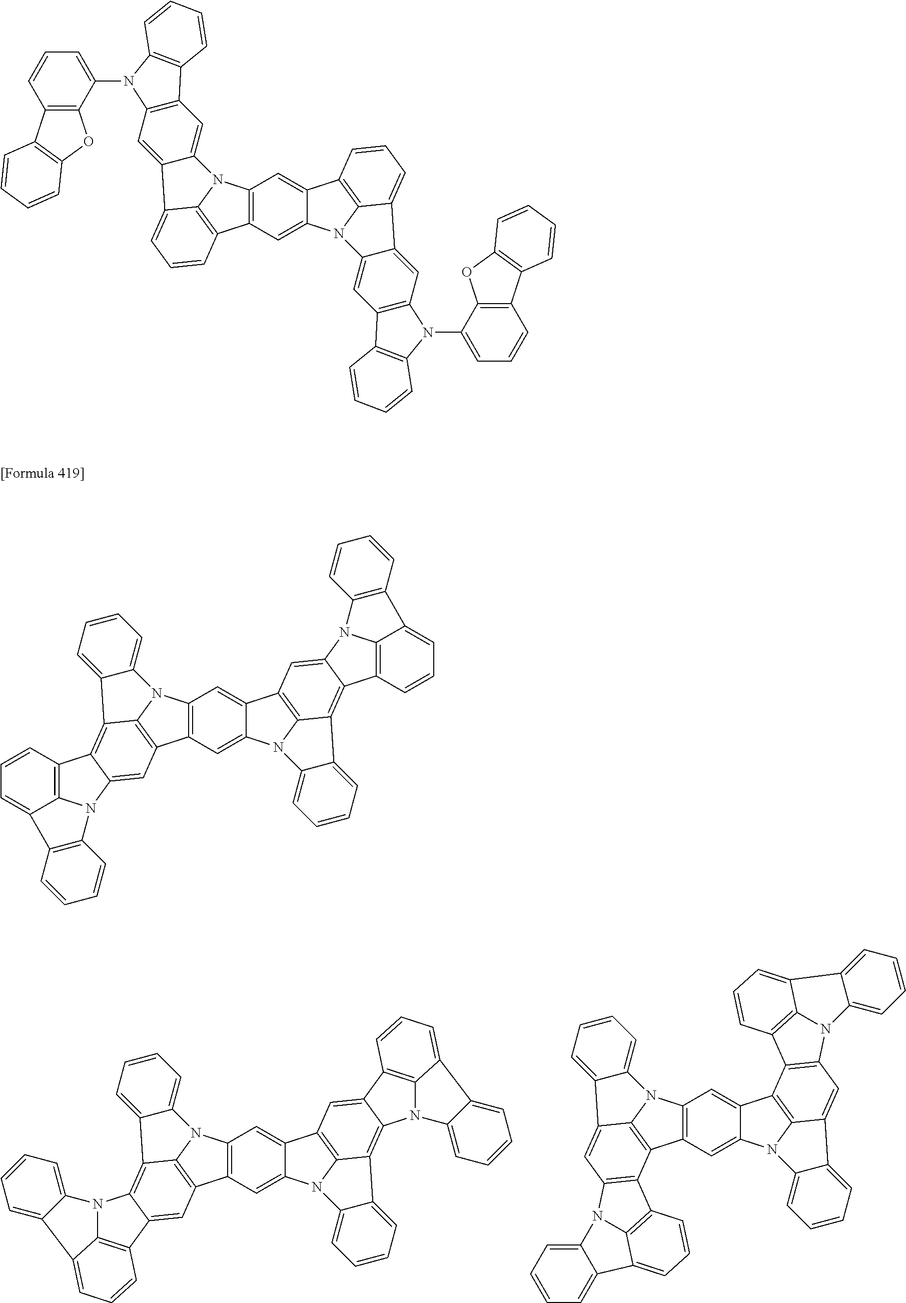 [SMILES: c1ccc2c(c1)oc1c(-n3c4ccccc4c4cc5c(cc43)c3cccc4c6cc7c(cc6n5c34)c3cccc4c5cc6c(cc5n7c43)c3ccccc3n6-c3cccc4c3oc3ccccc34)cccc12 ; c1ccc2c(c1)c1cccc3c4c5c6ccccc6n6c7cc8c9cc%10c(c%11cccc%12c%13ccccc%13n%10c%12%11)c%10c%11ccccc%11n(c8cc7c(cc4n2c13)c56)c9%10 ; c1ccc2c(c1)c1c3c(cc4c5cccc6c7ccccc7n(c65)c41)c1cc4c(cc1n23)c1cc2c3cccc5c6ccccc6n(c53)c2c2c3ccccc3n4c12 ; c1ccc2c(c1)c1cccc3c4c5c6cc7c(cc6n6c8ccccc8c(cc4n2c13)c56)c1c2c3cccc4c5ccccc5n(c2cc2c5ccccc5n7c21)c43]

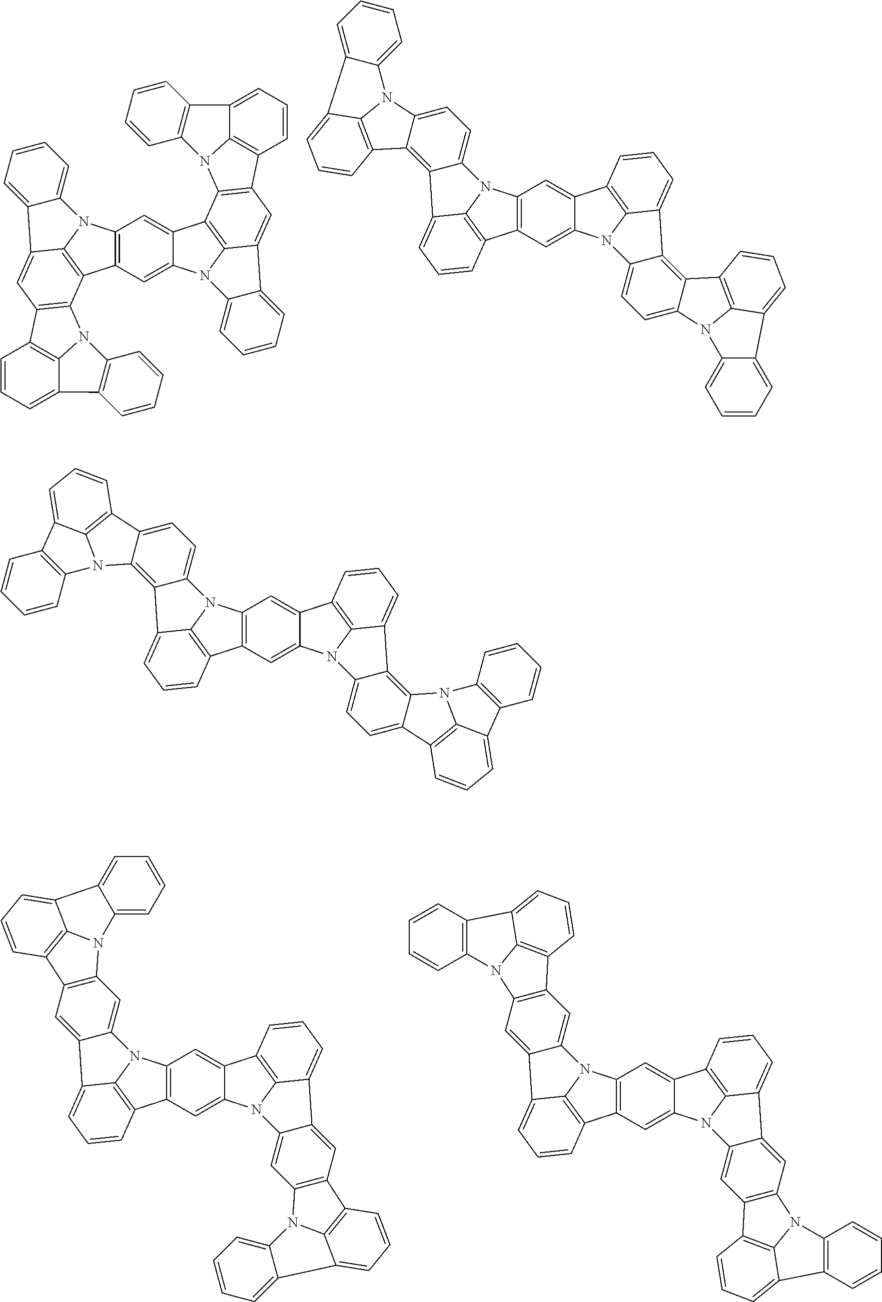

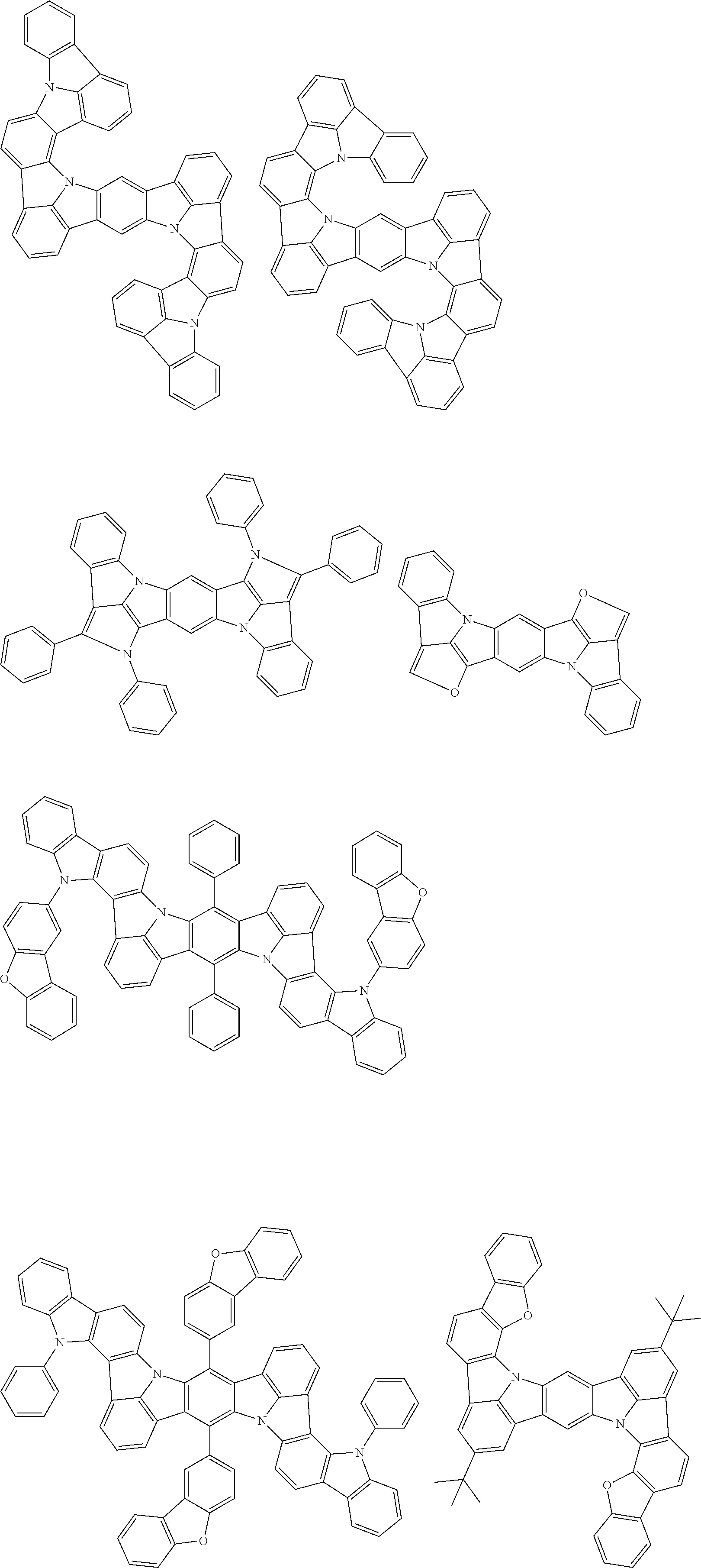

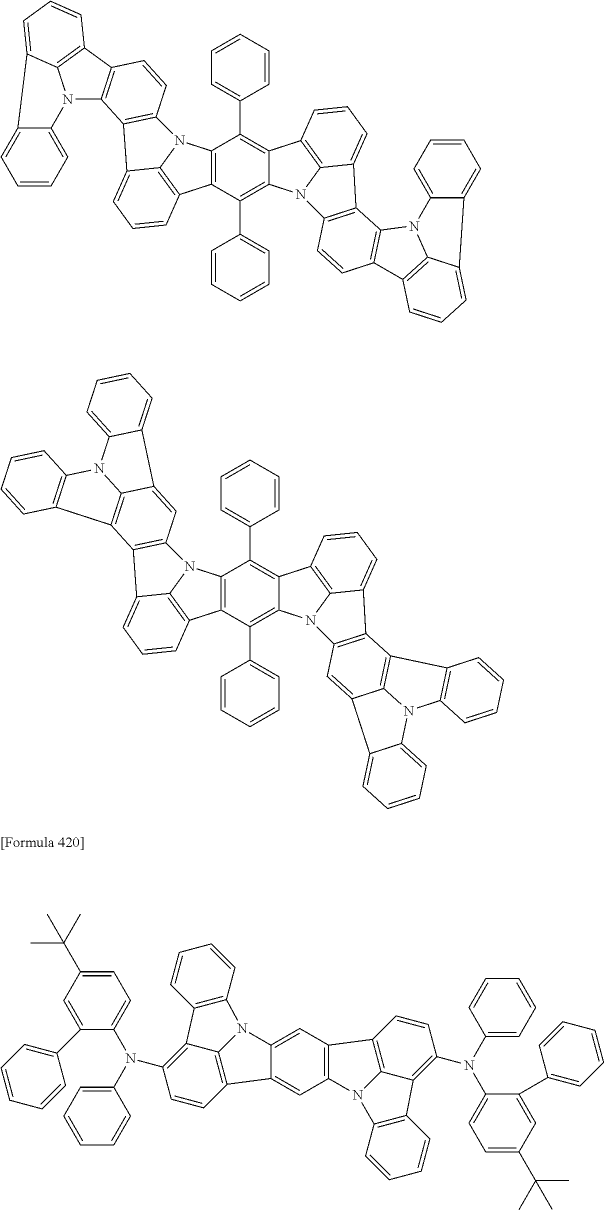 [SMILES: c1ccc(-c2c3c4cccc5c6c(ccc7c8cccc9c%10ccccc%10n(c98)c76)n(c3c(-c3ccccc3)c3c6cccc7c8c(ccc9c%10cccc%11c%12ccccc%12n(c%11%10)c98)n(c23)c67)c45)cc1 ; c1ccc(-c2c3c4cccc5c6c7c8ccccc8n8c9ccccc9c(cc6n(c3c(-c3ccccc3)c3c6cccc9c%10c%11c%12ccccc%12n%12c%13ccccc%13c(cc%10n(c23)c69)c%11%12)c45)c78)cc1 ; CC(C)(C)c1ccc(N(c2ccccc2)c2ccc3c4cc5c(cc4n4c6ccccc6c2c34)c2ccc(N(c3ccccc3)c3ccc(C(C)(C)C)cc3-c3ccccc3)c3c4ccccc4n5c23)c(-c2ccccc2)c1]

### Compound Represented by Formula (7)

The compound represented by the formula (7) will be described below.

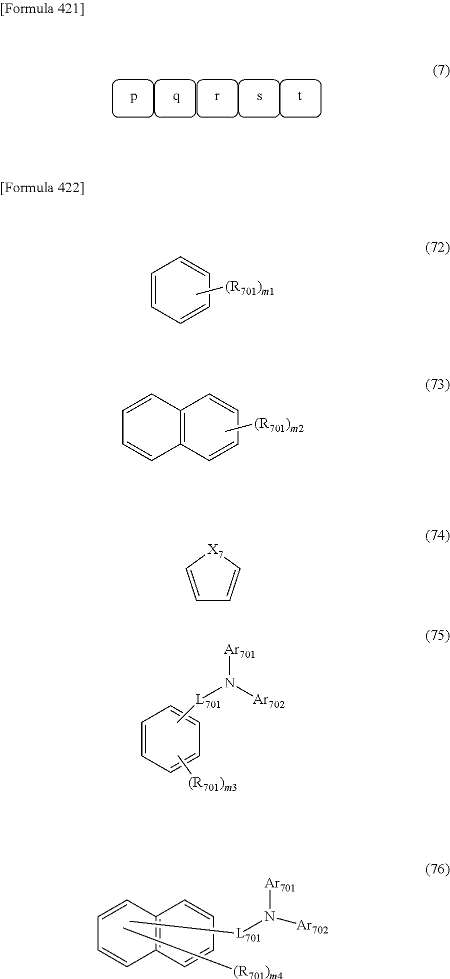

In the formula (7):
a ring r is a ring represented by the formula (72) or the formula (73), the ring r being fused with adjacent ring(s) at any position(s);
a ring q and a ring s are each independently a ring represented by the formula (74) and fused with adjacent ring(s) at any position(s);
a ring p and a ring t are each independently a structure represented by the formula (75) or the formula (76) and fused with adjacent ring(s) at any position(s);
X₇ is an oxygen atom, a sulfur atom, or NR₇₀₂;
when a plurality of R₇₀₁ are present, adjacent ones of the plurality of R₇₀₁ are mutually bonded to form a substituted or unsubstituted monocyclic ring, mutually bonded to form a substituted or unsubstituted fused ring, or not mutually bonded; and
R₇₀₁ forming neither the monocyclic ring nor the fused ring and R₇₀₂ are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms;
Ar₇₀₁ and Ar₇₀₂ are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
L₇₀₁ is a substituted or unsubstituted alkylene group having 1 to 50 carbon atoms, a substituted or unsubstituted alkenylene group having 2 to 50 carbon atoms, a substituted or unsubstituted alkynylene group having 2 to 50 carbon atoms, a substituted or unsubstituted cycloalkylene group having 3 to 50 ring carbon atoms, a substituted or unsubstituted arylene group having 6 to 50 ring carbon atoms, or a substituted or unsubstituted divalent heterocyclic group having 5 to 50 ring atoms;
m1 is 0, 1, or 2;
m2 is 0, 1, 2, 3, or 4;
m3 is each independently 0, 1, 2, or 3;
m4 is each independently 0, 1, 2, 3, 4, or 5;
when a plurality of R₇₀₁ are present, the plurality of R₇₀₁ are mutually the same or different;
when a plurality of X₇ are present, the plurality of X₇ are mutually the same or different;
when a plurality of R₇₀₂ are present, the plurality of R₇₀₂ are mutually the same or different;
when a plurality of Ar₇₀₁ are present, the plurality of Ar₇₀₁ are mutually the same or different;
when a plurality of Ar₇₀₂ are present, the plurality of Ar₇₀₂ are mutually the same or different; and
when a plurality of L₇₀₁ are present, the plurality of L₇₀₁ are mutually the same or different.

In the formula (7), each of the ring p, ring q, ring r, ring s, and ring t is fused with an adjacent ring(s) sharing two carbon atoms. The fused position and orientation are not limited but may be defined as required.

In an exemplary embodiment, in the formula (72) or the formula (73) representing the ring r, m1=0 or m2=0 is satisfied.

In an exemplary embodiment, the compound represented by the formula (7) is represented by any one of formulae (71-1) to (71-6) below.

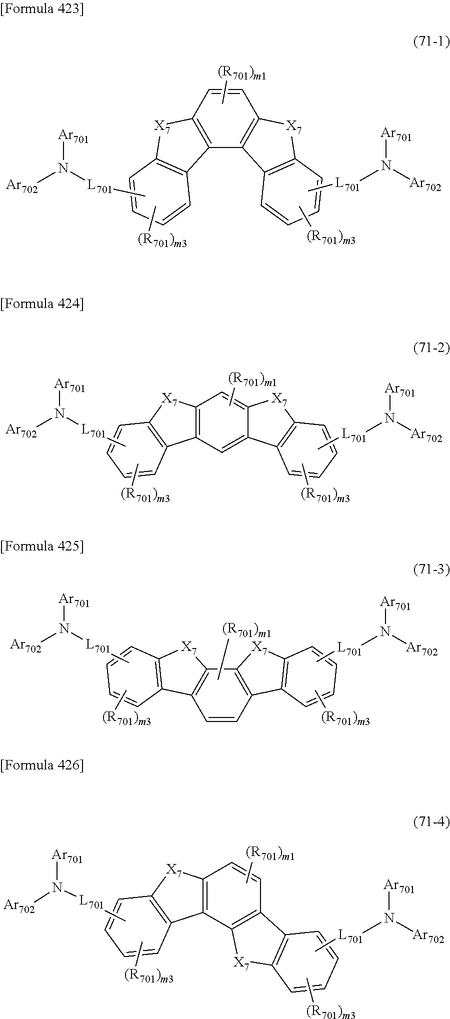

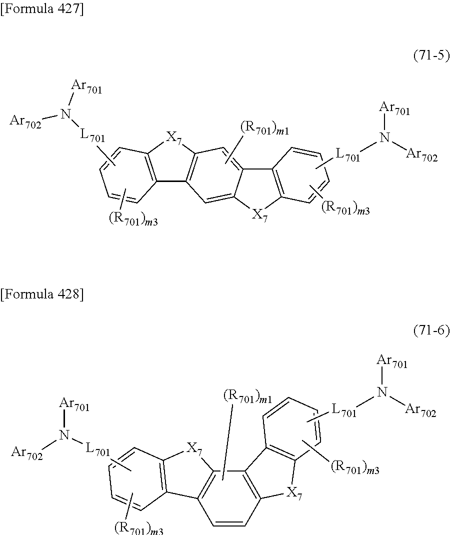

In the formulae (71-1) to (71-6), R₇₀₁, X₇, Ar₇₀₁, Ar₇₀₂, L₇₀₁, m1 and m3 respectively represent the same as R₇₀₁, X₇, Ar₇₀₁, Ar₇₀₂, L₇₀₁, m1 and m3 in the formula (7).

In an exemplary embodiment, the compound represented by the formula (7) is represented by any one of formulae (71-11) to (71-13) below.

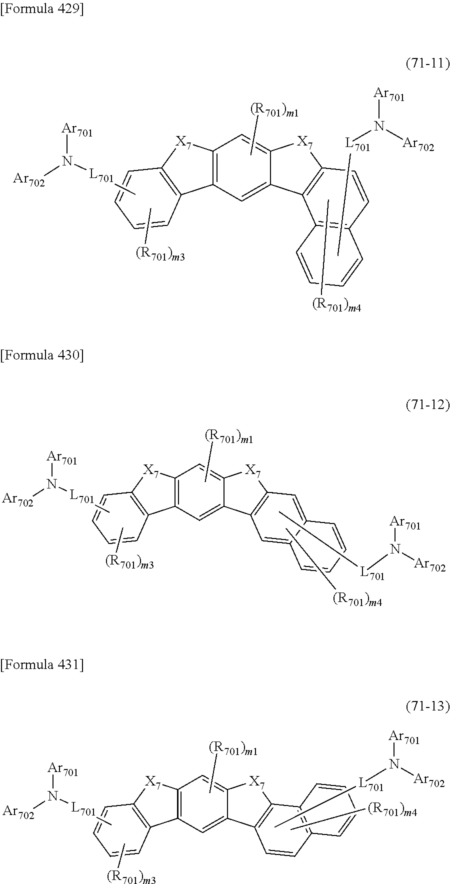

In the formulae (71-11) to (71-13), R₇₀₁, X₇, Ar₇₀₁, Ar₇₀₂, L₇₀₁, m1, m3 and m4 respectively represent the same as R₇₀₁, X₇, Ar₇₀₁, Ar₇₀₂, L₇₀₁, m1, m3 and m4 in the formula (7).

In an exemplary embodiment, the compound represented by the formula (7) is represented by any one of formulae (71-21) to (71-25) below.

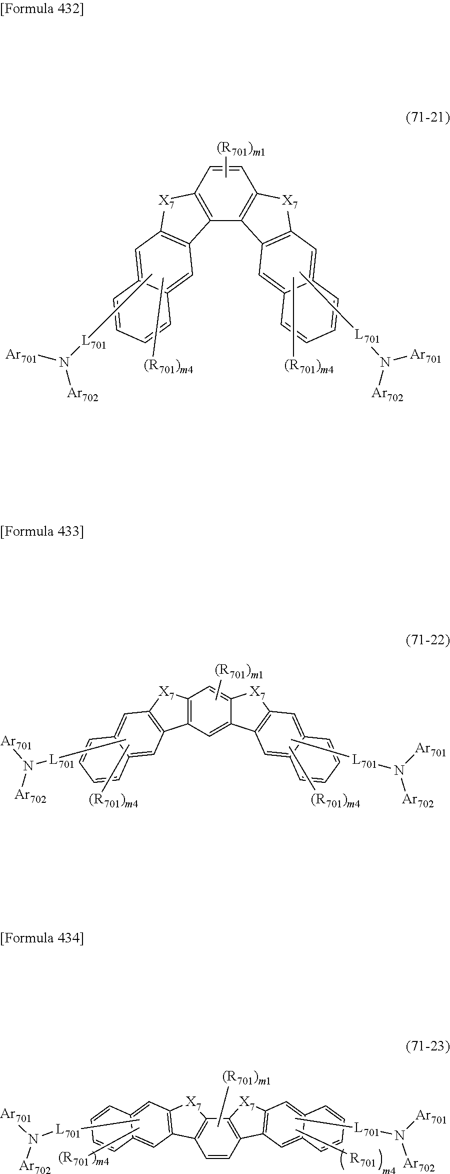

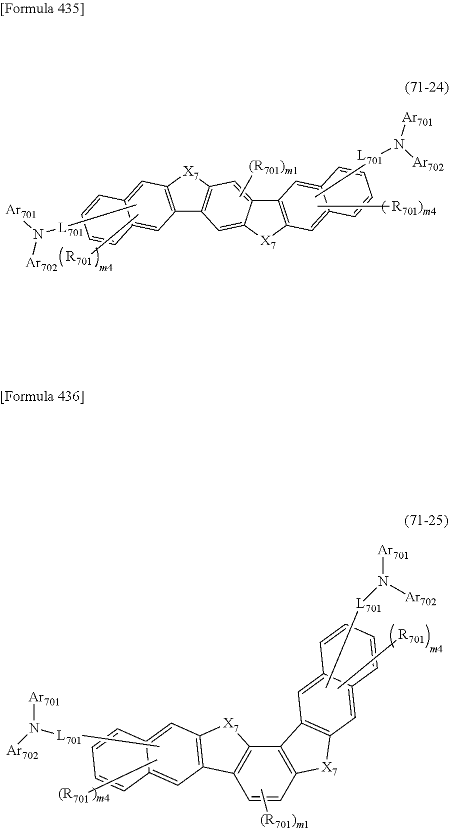

In the formulae (71-21) to (71-25), R₇₀₁, X₇, Ar₇₀₁, Ar₇₀₂, L₇₀₁, m1 and m4 respectively represent the same as R₇₀₁, X₇, Ar₇₀₁, Ar₇₀₂, L₇₀₁, m1 and m4 in the formula (7).

In an exemplary embodiment, the compound represented by the formula (7) is represented by any one of formulae (71-31) to (71-33) below.

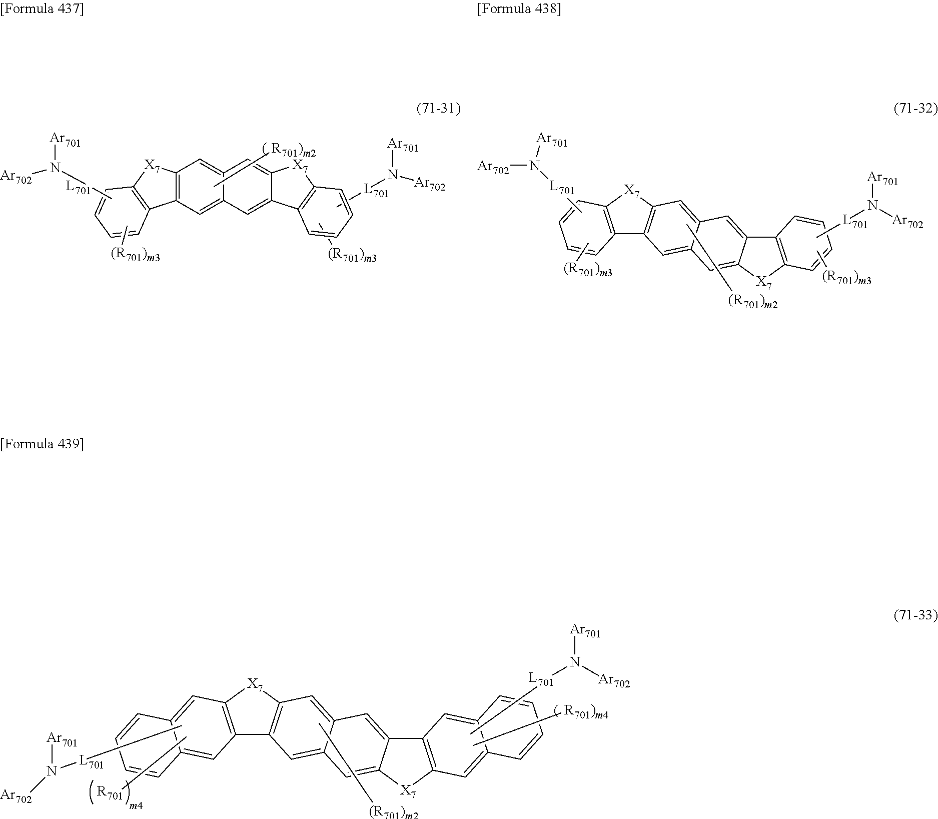

In the formulae (71-31) to (71-33), R₇₀₁, X₇, Ar₇₀₁, Ar₇₀₂, L₇₀₁, and m2 to m4 respectively represent the same as R₇₀₁, X₇, Ar₇₀₁, Ar₇₀₂, L₇₀₁, and m2 to m4 in the formula (7).

In an exemplary embodiment, Ar₇₀₁ and Ar₇₀₂ are each independently a substituted or unsubstituted aryl group having 6 to 50 ring carbon atoms.

In an exemplary embodiment, one of Ar₇₀₁ and Ar₇₀₂ is a substituted or unsubstituted aryl group having 6 to 50 ring carbon atoms, and the other of Ar₇₀₁ and Ar₇₀₂ is a substituted or unsubstituted heterocyclic group having 5 to 50 ring atoms.

### Specific Examples of Compound Represented by Formula (7)

Specific examples of the compound represented by the formula (7) include compounds as below.

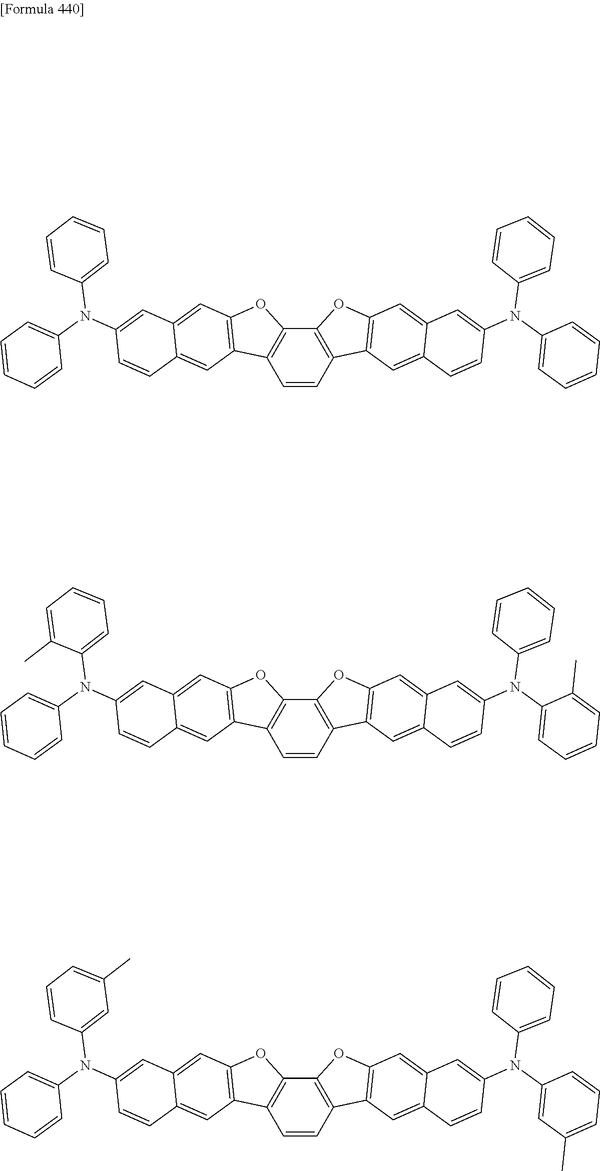 [SMILES: c1ccc(N(c2ccccc2)c2ccc3cc4c(cc3c2)oc2c4ccc3c4cc5ccc(N(c6ccccc6)c6ccccc6)cc5cc4oc32)cc1 ; Cc1ccccc1N(c1ccccc1)c1ccc2cc3c(cc2c1)oc1c3ccc2c3cc4ccc(N(c5ccccc5)c5ccccc5C)cc4cc3oc21 ; Cc1cccc(N(c2ccccc2)c2ccc3cc4c(cc3c2)oc2c4ccc3c4cc5ccc(N(c6ccccc6)c6cccc(C)c6)cc5cc4oc32)c1]

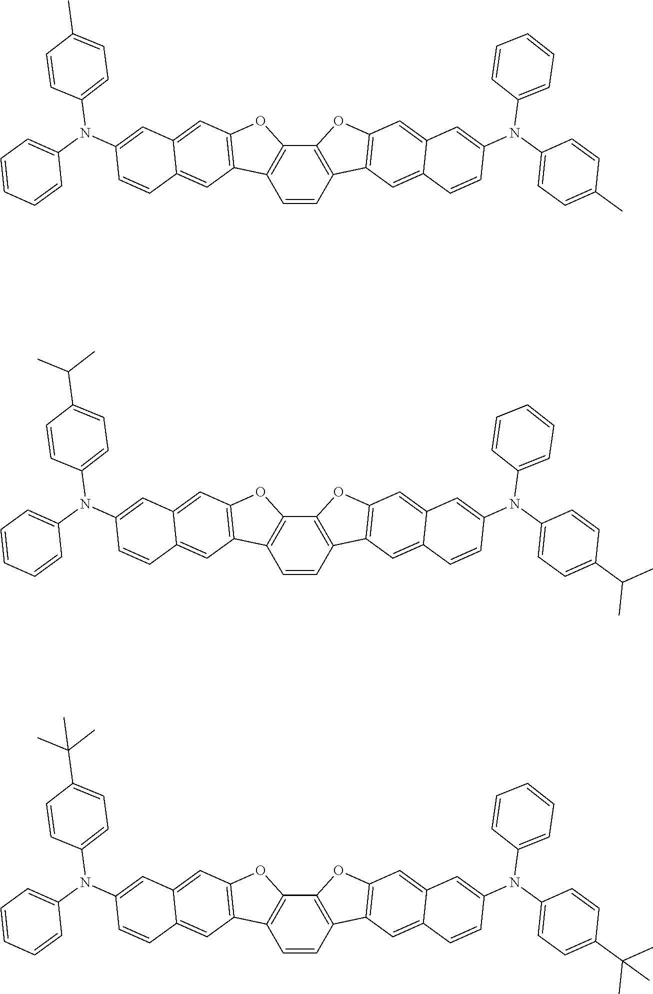 [SMILES: Cc1ccc(N(c2ccccc2)c2ccc3cc4c(cc3c2)oc2c4ccc3c4cc5ccc(N(c6ccccc6)c6ccc(C)cc6)cc5cc4oc32)cc1 ; CC(C)c1ccc(N(c2ccccc2)c2ccc3cc4c(cc3c2)oc2c4ccc3c4cc5ccc(N(c6ccccc6)c6ccc(C(C)C)cc6)cc5cc4oc32)cc1 ; CC(C)(C)c1ccc(N(c2ccccc2)c2ccc3cc4c(cc3c2)oc2c4ccc3c4cc5ccc(N(c6ccccc6)c6ccc(C(C)(C)C)cc6)cc5cc4oc32)cc1]

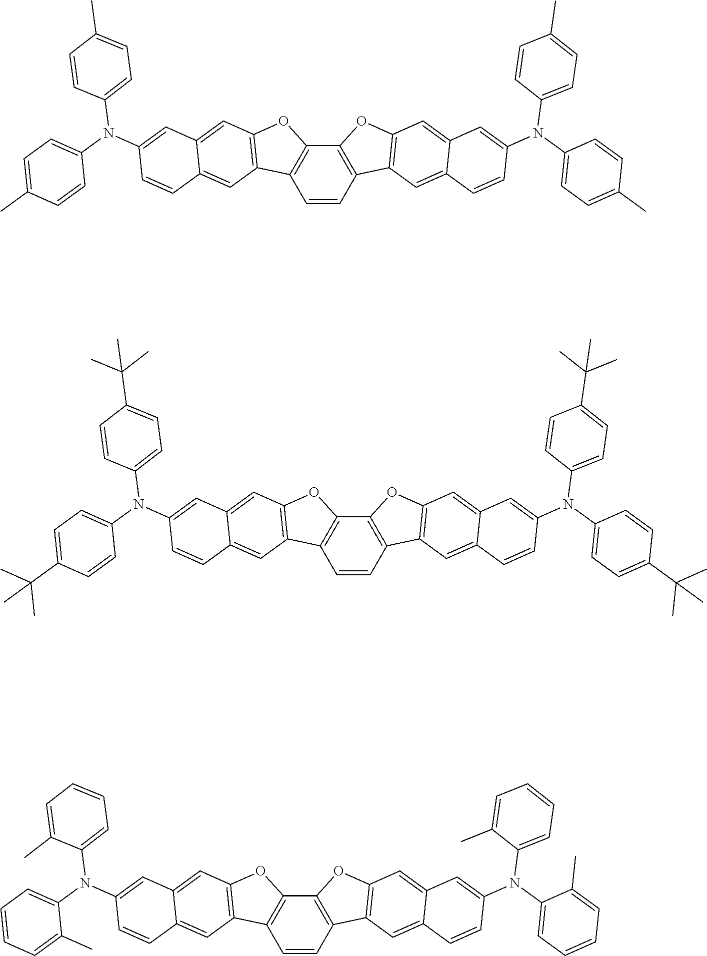

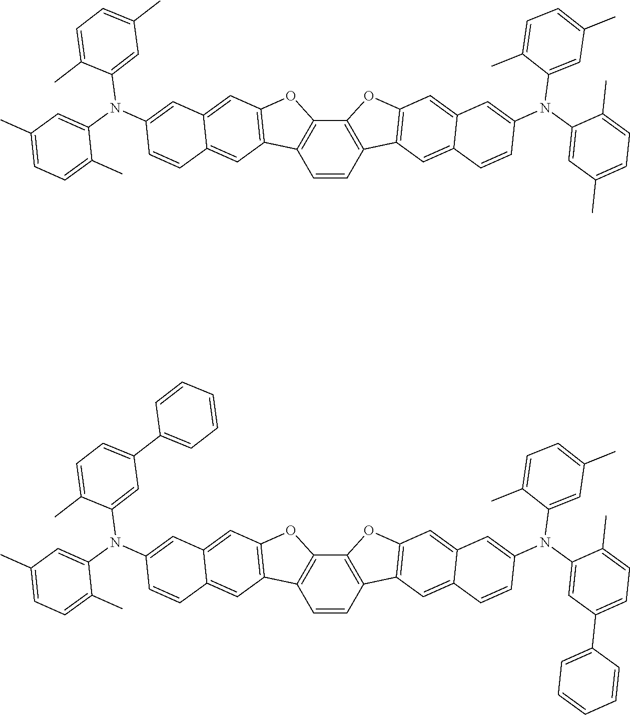 [SMILES: Cc1ccc(C)c(N(c2ccc3cc4c(cc3c2)oc2c4ccc3c4cc5ccc(N(c6cc(C)ccc6C)c6cc(C)ccc6C)cc5cc4oc32)c2cc(C)ccc2C)c1 ; Cc1ccc(C)c(N(c2ccc3cc4c(cc3c2)oc2c4ccc3c4cc5ccc(N(c6cc(C)ccc6C)c6cc(-c7ccccc7)ccc6C)cc5cc4oc32)c2cc(-c3ccccc3)ccc2C)c1]

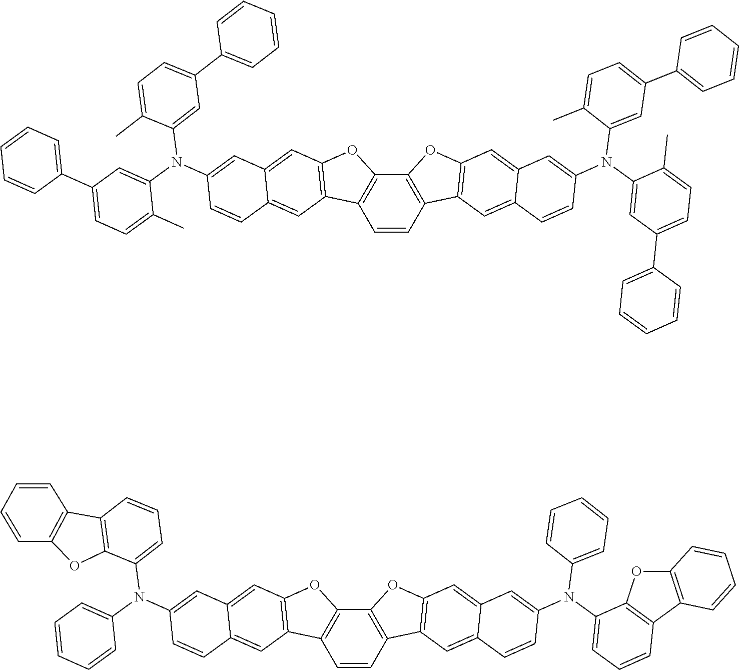 [SMILES: Cc1ccc(-c2ccccc2)cc1N(c1ccc2cc3c(cc2c1)oc1c3ccc2c3cc4ccc(N(c5cc(-c6ccccc6)ccc5C)c5cc(-c6ccccc6)ccc5C)cc4cc3oc21)c1cc(-c2ccccc2)ccc1C ; c1ccc(N(c2ccc3cc4c(cc3c2)oc2c4ccc3c4cc5ccc(N(c6ccccc6)c6cccc7c6oc6ccccc67)cc5cc4oc32)c2cccc3c2oc2ccccc23)cc1]

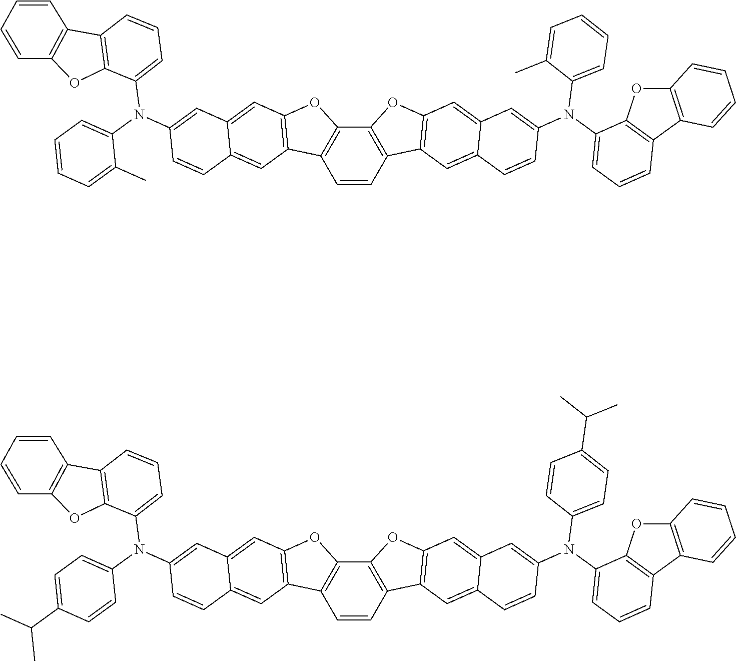 [SMILES: Cc1ccccc1N(c1ccc2cc3c(cc2c1)oc1c3ccc2c3cc4ccc(N(c5ccccc5C)c5cccc6c5oc5ccccc56)cc4cc3oc21)c1cccc2c1oc1ccccc12 ; CC(C)c1ccc(N(c2ccc3cc4c(cc3c2)oc2c4ccc3c4cc5ccc(N(c6ccc(C(C)C)cc6)c6cccc7c6oc6ccccc67)cc5cc4oc32)c2cccc3c2oc2ccccc23)cc1]

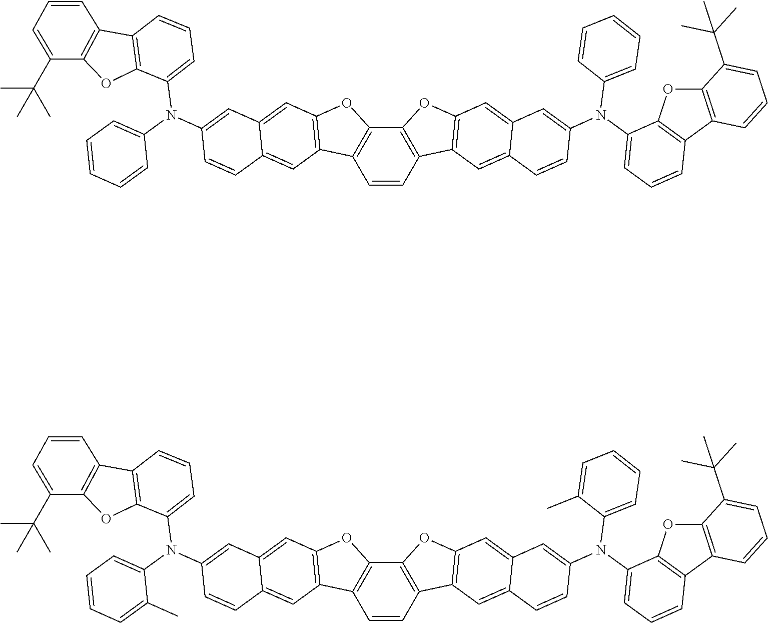 [SMILES: CC(C)(C)c1cccc2c1oc1c(N(c3ccccc3)c3ccc4cc5c(cc4c3)oc3c5ccc4c5cc6ccc(N(c7ccccc7)c7cccc8c7oc7c(C(C)(C)C)cccc78)cc6cc5oc43)cccc12 ; Cc1ccccc1N(c1ccc2cc3c(cc2c1)oc1c3ccc2c3cc4ccc(N(c5ccccc5C)c5cccc6c5oc5c(C(C)(C)C)cccc56)cc4cc3oc21)c1cccc2c1oc1c(C(C)(C)C)cccc12]

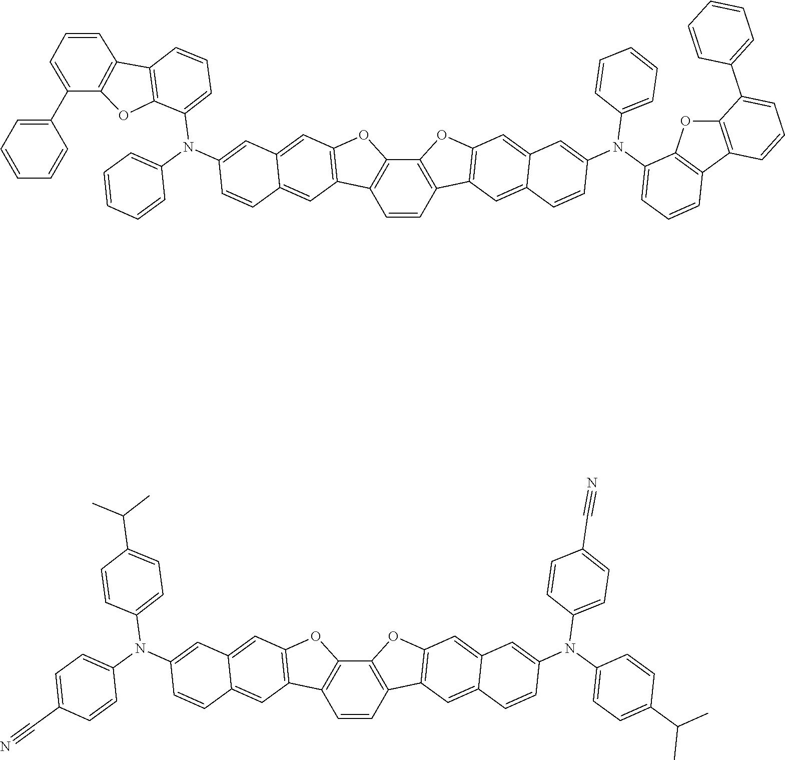 [SMILES: c1ccc(-c2cccc3c2oc2c(N(c4ccccc4)c4ccc5cc6c(cc5c4)oc4c6ccc5c6cc7ccc(N(c8ccccc8)c8cccc9c8oc8c(-c%10ccccc%10)cccc89)cc7cc6oc54)cccc23)cc1 ; CC(C)c1ccc(N(c2ccc(C#N)cc2)c2ccc3cc4c(cc3c2)oc2c4ccc3c4cc5ccc(N(c6ccc(C#N)cc6)c6ccc(C(C)C)cc6)cc5cc4oc32)cc1]

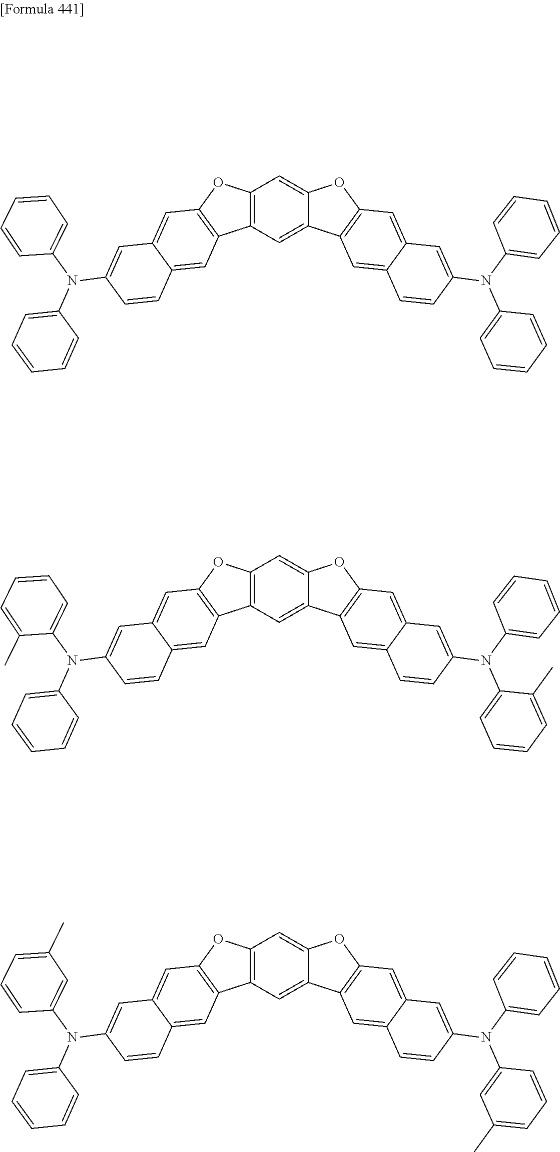 [SMILES: c1ccc(N(c2ccccc2)c2ccc3cc4c(cc3c2)oc2cc3oc5cc6cc(N(c7ccccc7)c7ccccc7)ccc6cc5c3cc24)cc1 ; Cc1ccccc1N(c1ccccc1)c1ccc2cc3c(cc2c1)oc1cc2oc4cc5cc(N(c6ccccc6)c6ccccc6C)ccc5cc4c2cc13 ; Cc1cccc(N(c2ccccc2)c2ccc3cc4c(cc3c2)oc2cc3oc5cc6cc(N(c7ccccc7)c7cccc(C)c7)ccc6cc5c3cc24)c1]

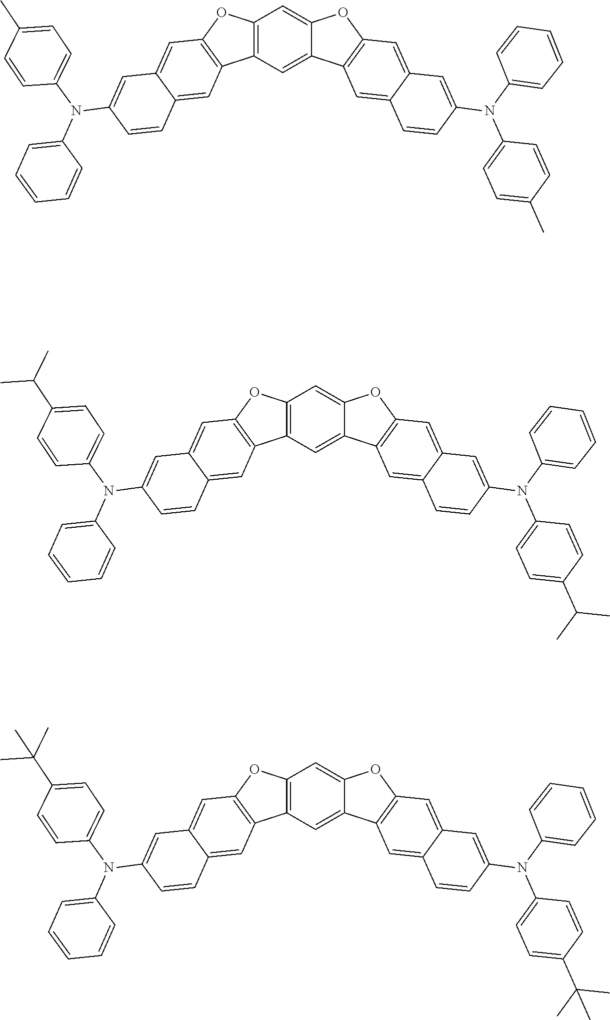

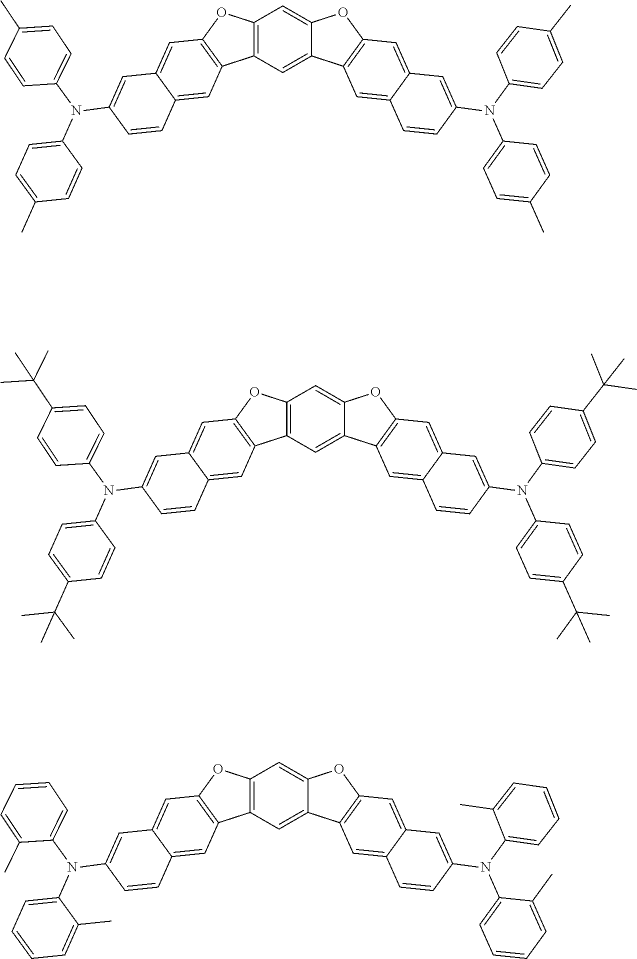

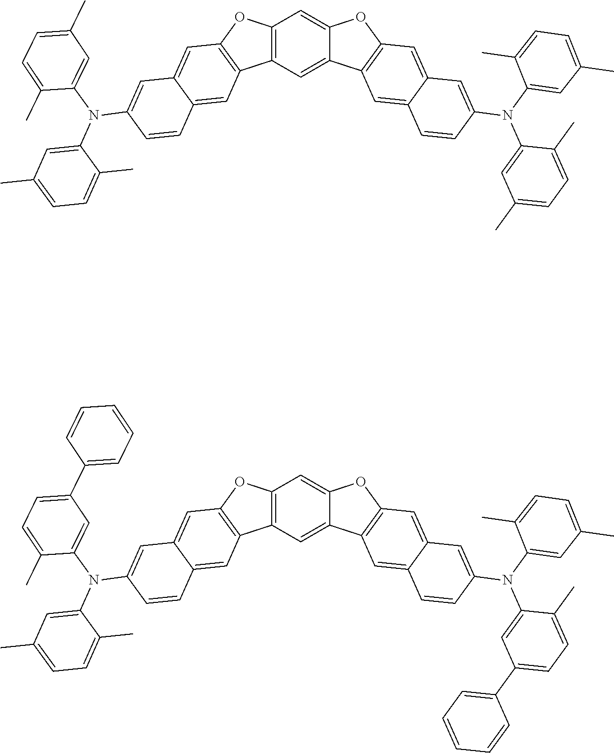

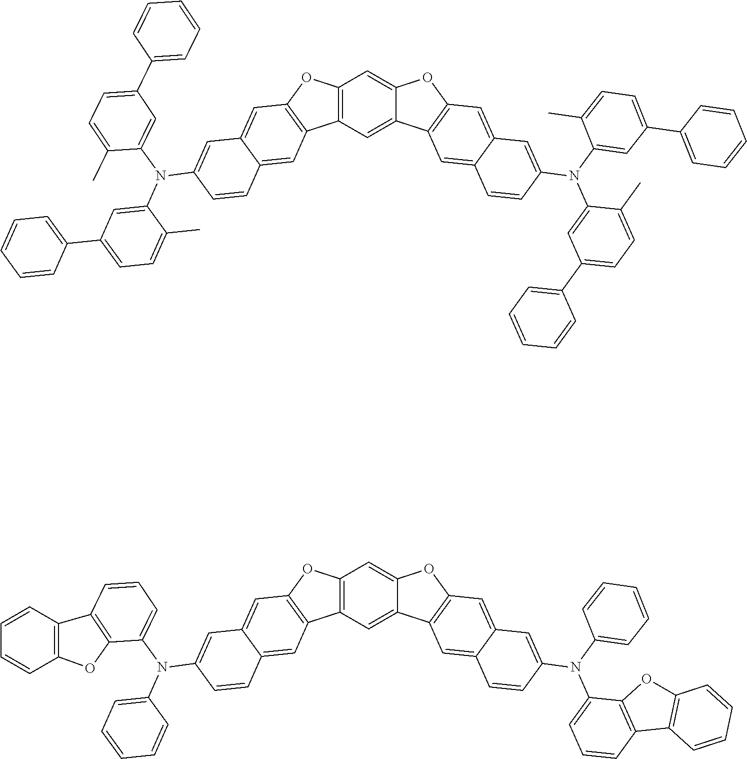 [SMILES: Cc1ccc(-c2ccccc2)cc1N(c1ccc2cc3c(cc2c1)oc1cc2oc4cc5cc(N(c6cc(-c7ccccc7)ccc6C)c6cc(-c7ccccc7)ccc6C)ccc5cc4c2cc13)c1cc(-c2ccccc2)ccc1C ; c1ccc(N(c2ccc3cc4c(cc3c2)oc2cc3oc5cc6cc(N(c7ccccc7)c7cccc8c7oc7ccccc78)ccc6cc5c3cc24)c2cccc3c2oc2ccccc23)cc1]

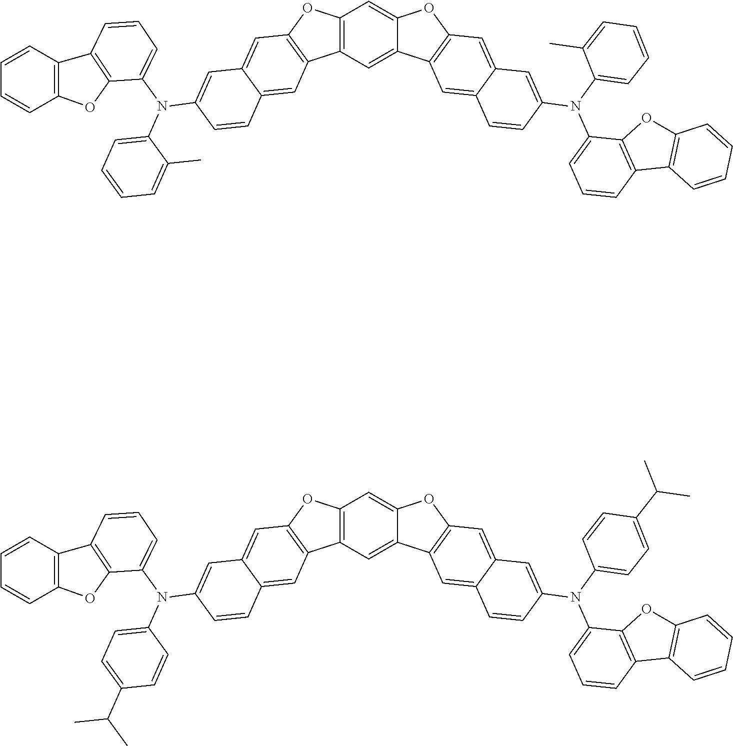 [SMILES: Cc1ccccc1N(c1ccc2cc3c(cc2c1)oc1cc2oc4cc5cc(N(c6ccccc6C)c6cccc7c6oc6ccccc67)ccc5cc4c2cc13)c1cccc2c1oc1ccccc12 ; CC(C)c1ccc(N(c2ccc3cc4c(cc3c2)oc2cc3oc5cc6cc(N(c7ccc(C(C)C)cc7)c7cccc8c7oc7ccccc78)ccc6cc5c3cc24)c2cccc3c2oc2ccccc23)cc1]

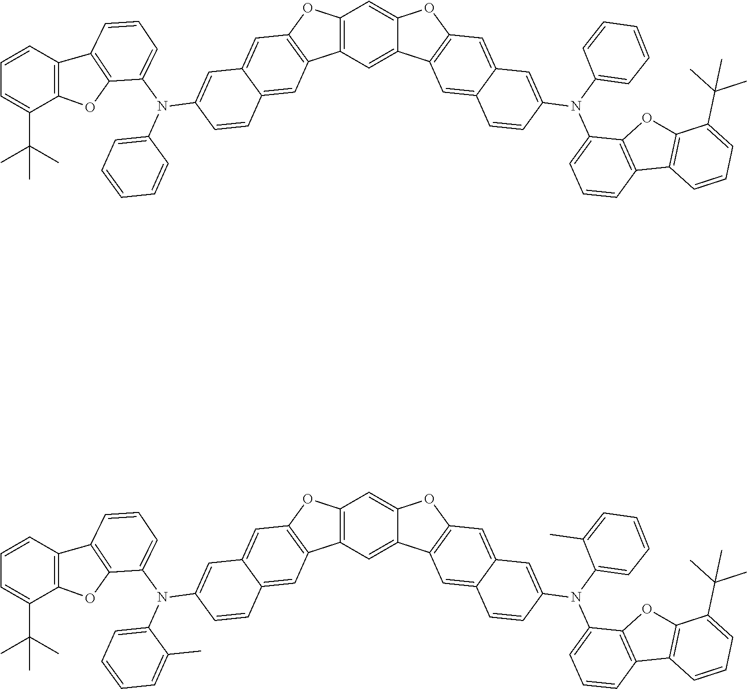

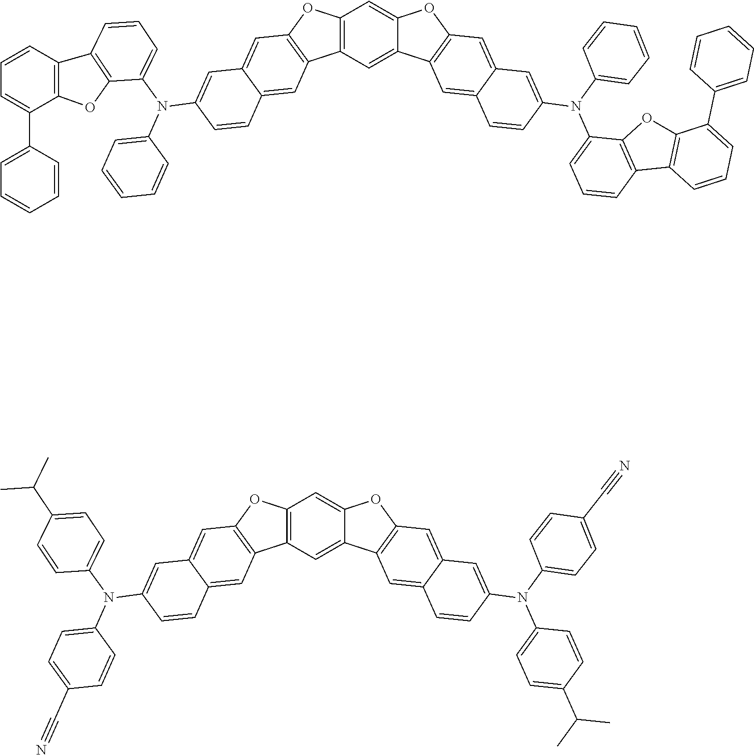 [SMILES: c1ccc(-c2cccc3c2oc2c(N(c4ccccc4)c4ccc5cc6c(cc5c4)oc4cc5oc7cc8cc(N(c9ccccc9)c9cccc%10c9oc9c(-c%11ccccc%11)cccc9%10)ccc8cc7c5cc46)cccc23)cc1 ; CC(C)c1ccc(N(c2ccc(C#N)cc2)c2ccc3cc4c(cc3c2)oc2cc3oc5cc6cc(N(c7ccc(C#N)cc7)c7ccc(C(C)C)cc7)ccc6cc5c3cc24)cc1]

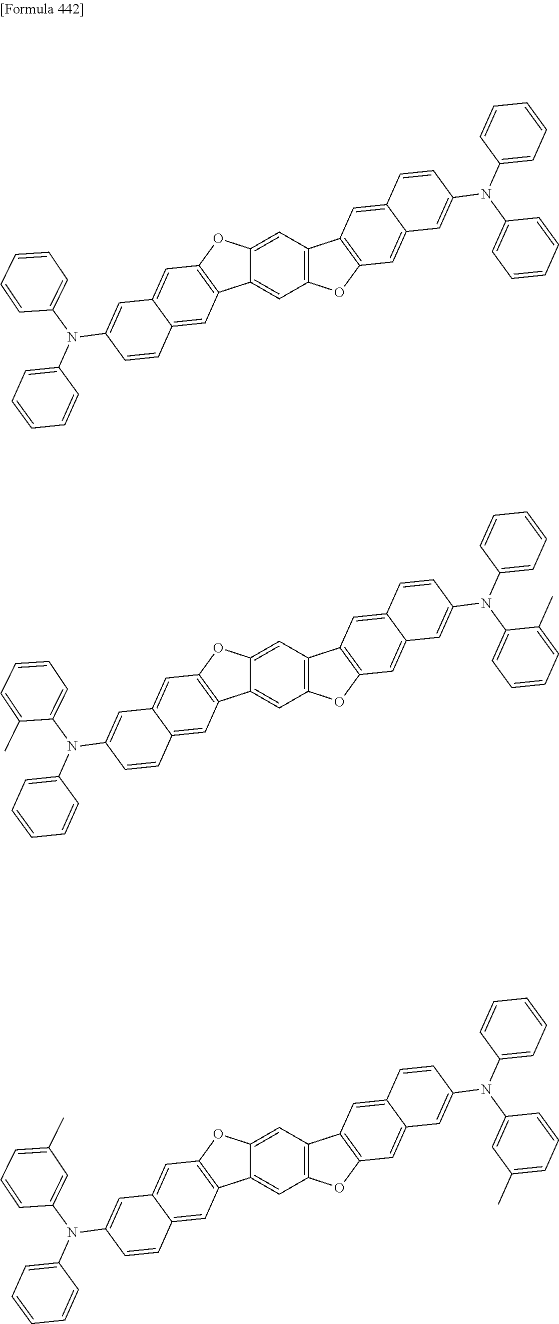 [SMILES: c1ccc(N(c2ccccc2)c2ccc3cc4c(cc3c2)oc2cc3c(cc24)oc2cc4cc(N(c5ccccc5)c5ccccc5)ccc4cc23)cc1 ; Cc1ccccc1N(c1ccccc1)c1ccc2cc3c(cc2c1)oc1cc2c(cc13)oc1cc3cc(N(c4ccccc4)c4ccccc4C)ccc3cc12 ; Cc1cccc(N(c2ccccc2)c2ccc3cc4c(cc3c2)oc2cc3c(cc24)oc2cc4cc(N(c5ccccc5)c5cccc(C)c5)ccc4cc23)c1]

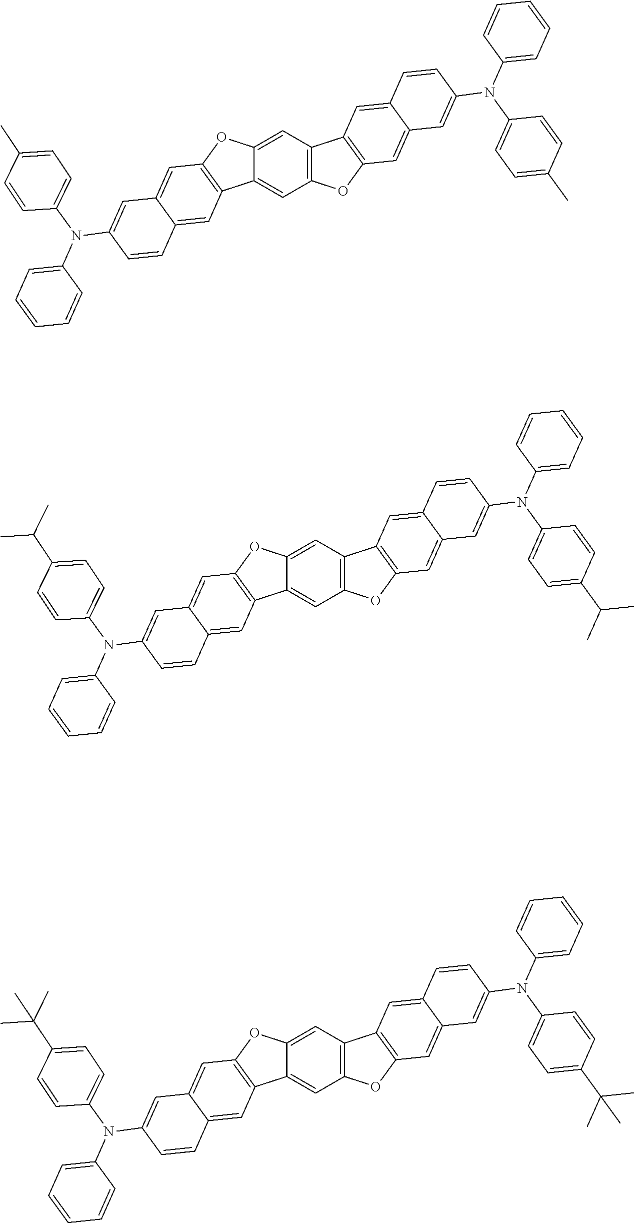

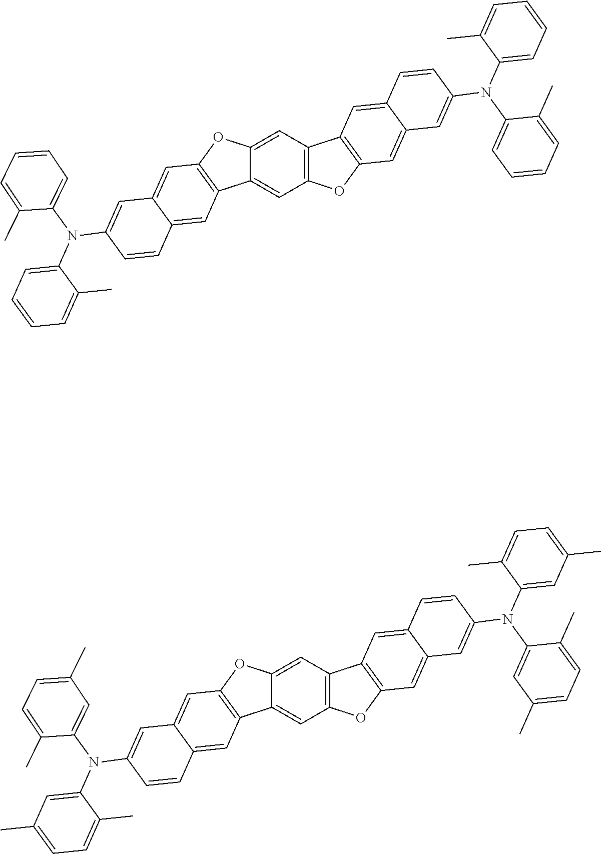

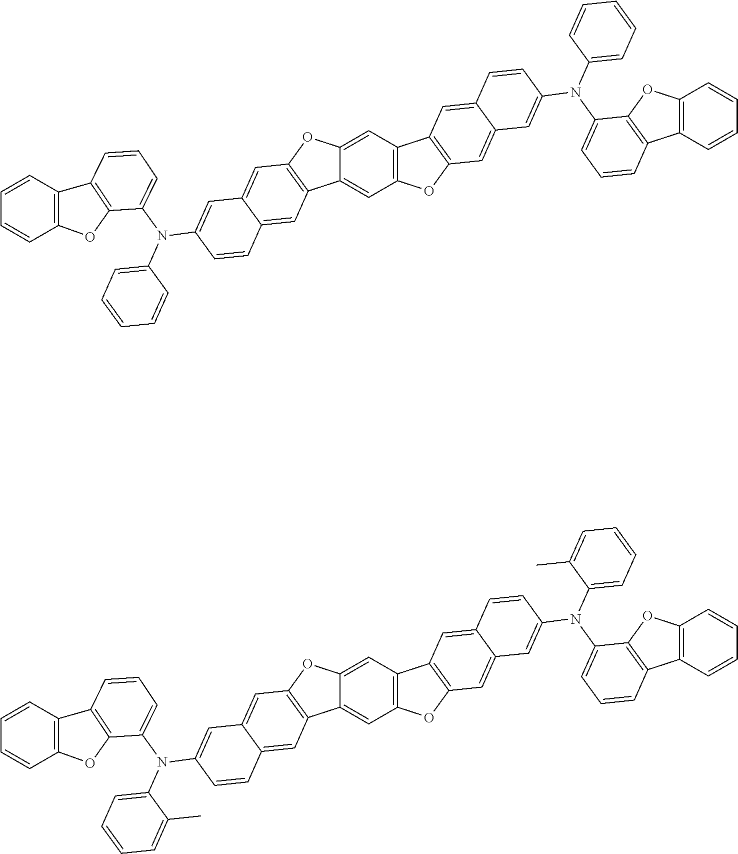 [SMILES: c1ccc(N(c2ccc3cc4c(cc3c2)oc2cc3c(cc24)oc2cc4cc(N(c5ccccc5)c5cccc6c5oc5ccccc56)ccc4cc23)c2cccc3c2oc2ccccc23)cc1 ; Cc1ccccc1N(c1ccc2cc3c(cc2c1)oc1cc2c(cc13)oc1cc3cc(N(c4ccccc4C)c4cccc5c4oc4ccccc45)ccc3cc12)c1cccc2c1oc1ccccc12]

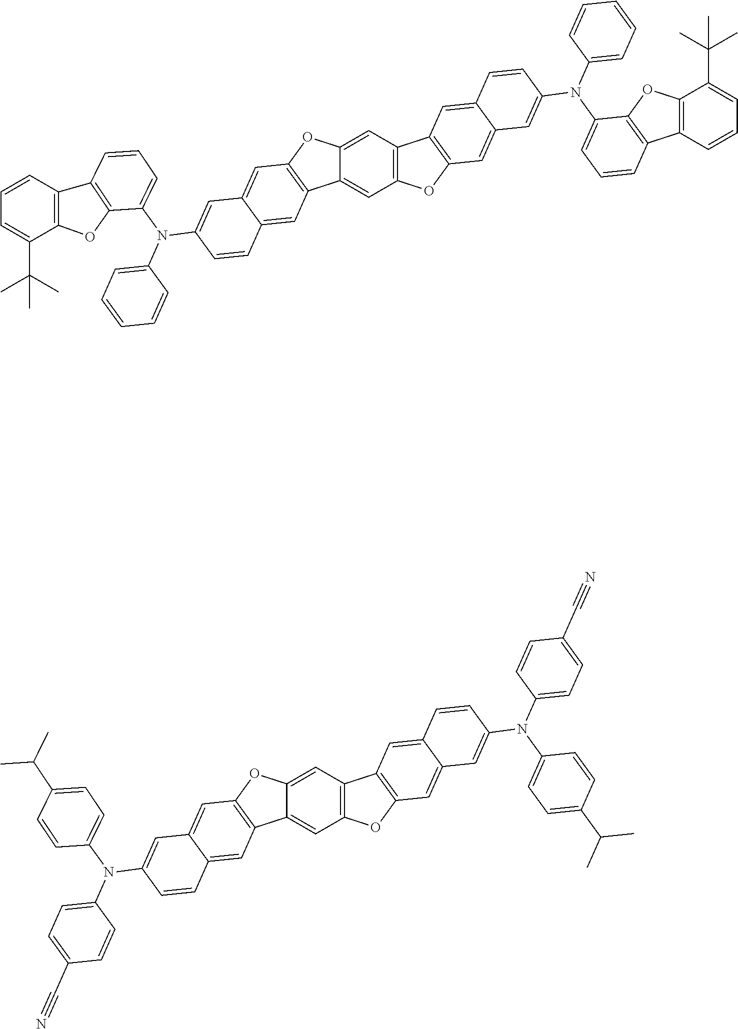

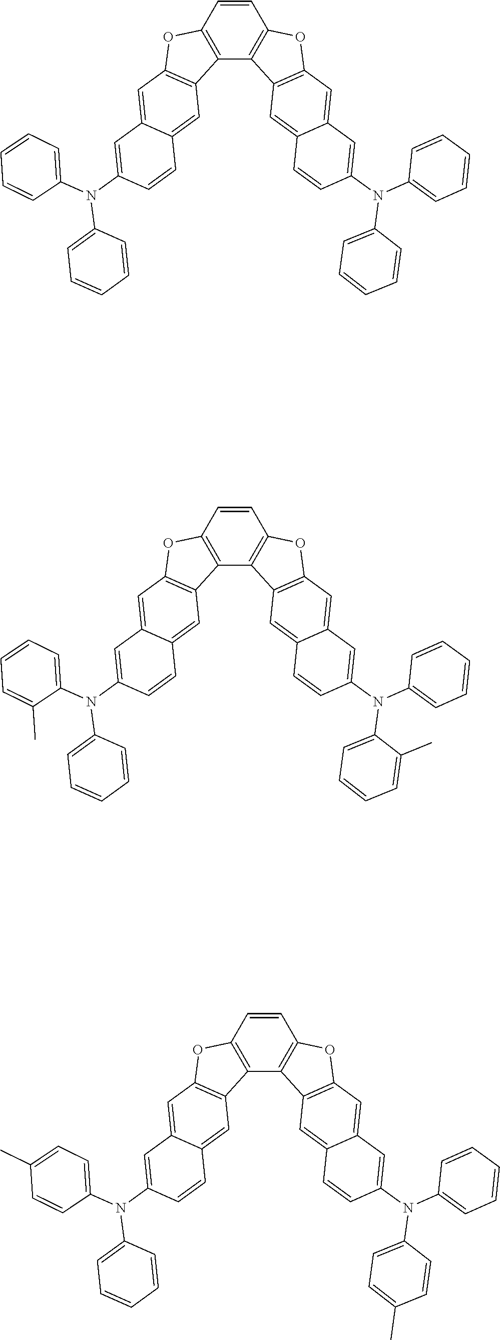 [SMILES: c1ccc(N(c2ccccc2)c2ccc3cc4c(cc3c2)oc2ccc3oc5cc6cc(N(c7ccccc7)c7ccccc7)ccc6cc5c3c24)cc1 ; Cc1ccccc1N(c1ccccc1)c1ccc2cc3c(cc2c1)oc1ccc2oc4cc5cc(N(c6ccccc6)c6ccccc6C)ccc5cc4c2c13 ; Cc1ccc(N(c2ccccc2)c2ccc3cc4c(cc3c2)oc2ccc3oc5cc6cc(N(c7ccccc7)c7ccc(C)cc7)ccc6cc5c3c24)cc1]

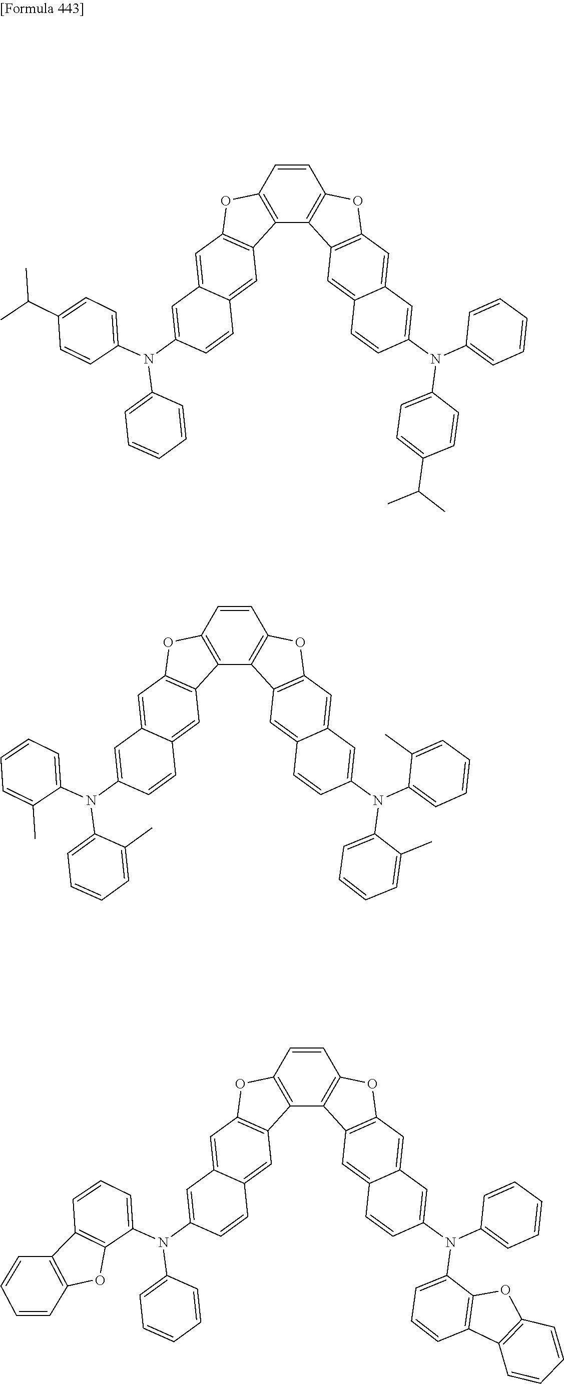 [SMILES: CC(C)c1ccc(N(c2ccccc2)c2ccc3cc4c(cc3c2)oc2ccc3oc5cc6cc(N(c7ccccc7)c7ccc(C(C)C)cc7)ccc6cc5c3c24)cc1 ; Cc1ccccc1N(c1ccc2cc3c(cc2c1)oc1ccc2oc4cc5cc(N(c6ccccc6C)c6ccccc6C)ccc5cc4c2c13)c1ccccc1C ; c1ccc(N(c2ccc3cc4c(cc3c2)oc2ccc3oc5cc6cc(N(c7ccccc7)c7cccc8c7oc7ccccc78)ccc6cc5c3c24)c2cccc3c2oc2ccccc23)cc1]

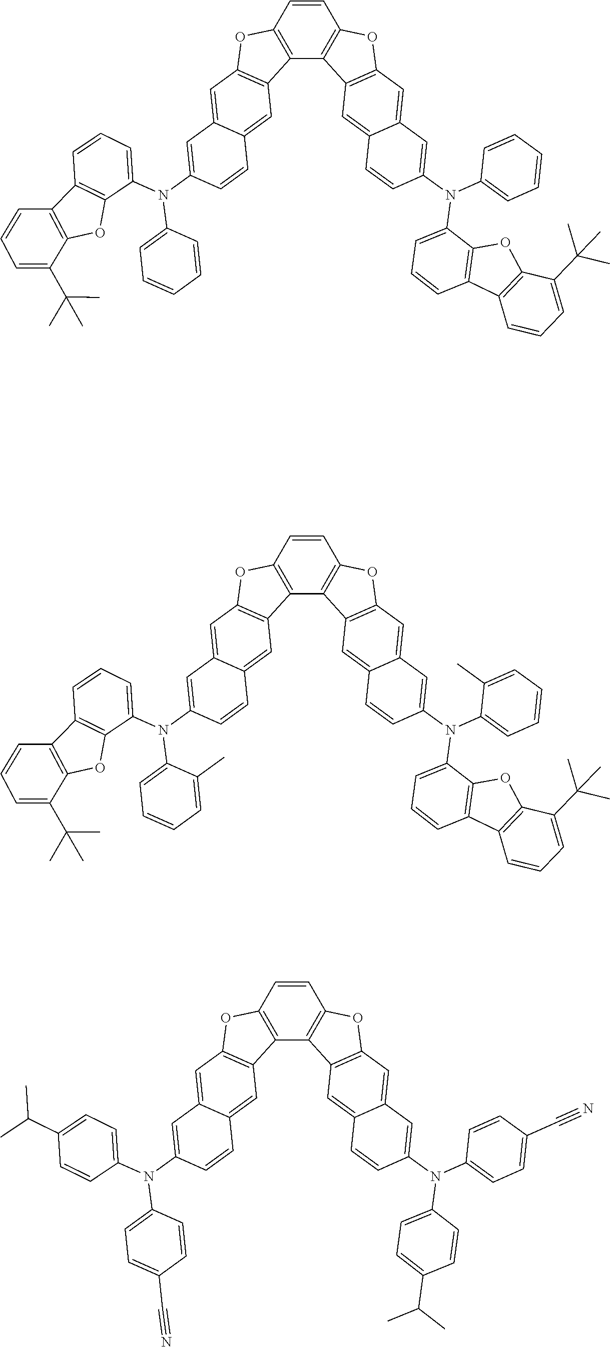

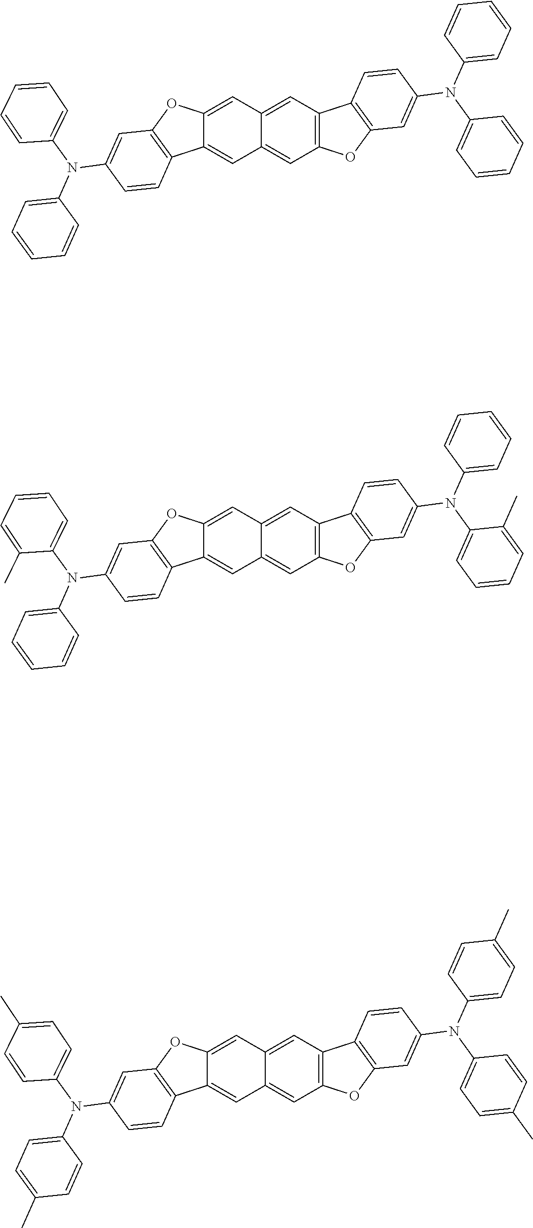

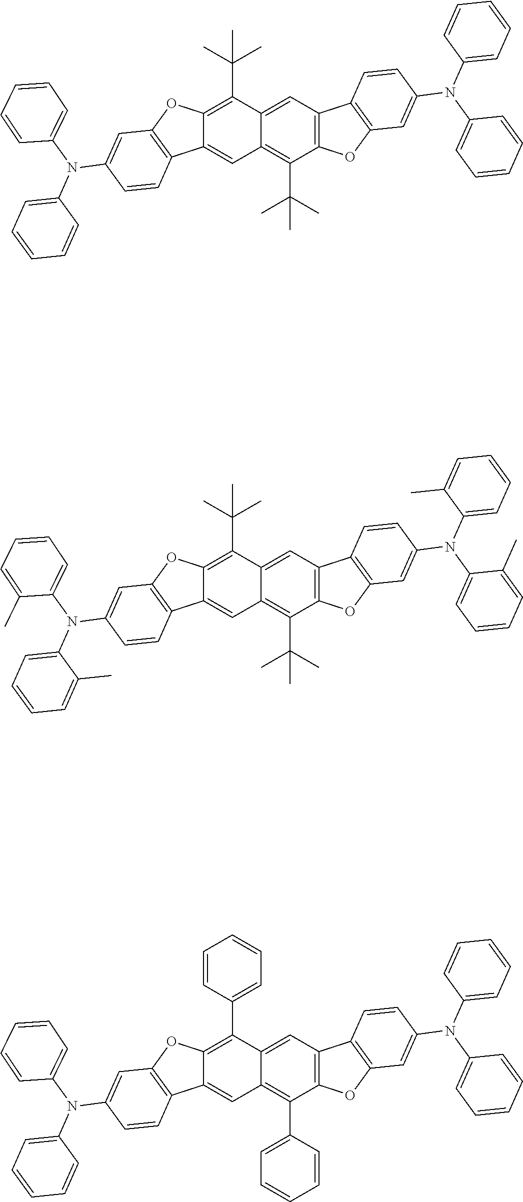 [SMILES: CC(C)(C)c1c2cc3c(oc4cc(N(c5ccccc5)c5ccccc5)ccc43)c(C(C)(C)C)c2cc2c1oc1cc(N(c3ccccc3)c3ccccc3)ccc12 ; Cc1ccccc1N(c1ccc2c(c1)oc1c(C(C)(C)C)c3cc4c(oc5cc(N(c6ccccc6C)c6ccccc6C)ccc54)c(C(C)(C)C)c3cc12)c1ccccc1C ; c1ccc(-c2c3cc4c(oc5cc(N(c6ccccc6)c6ccccc6)ccc54)c(-c4ccccc4)c3cc3c2oc2cc(N(c4ccccc4)c4ccccc4)ccc23)cc1]

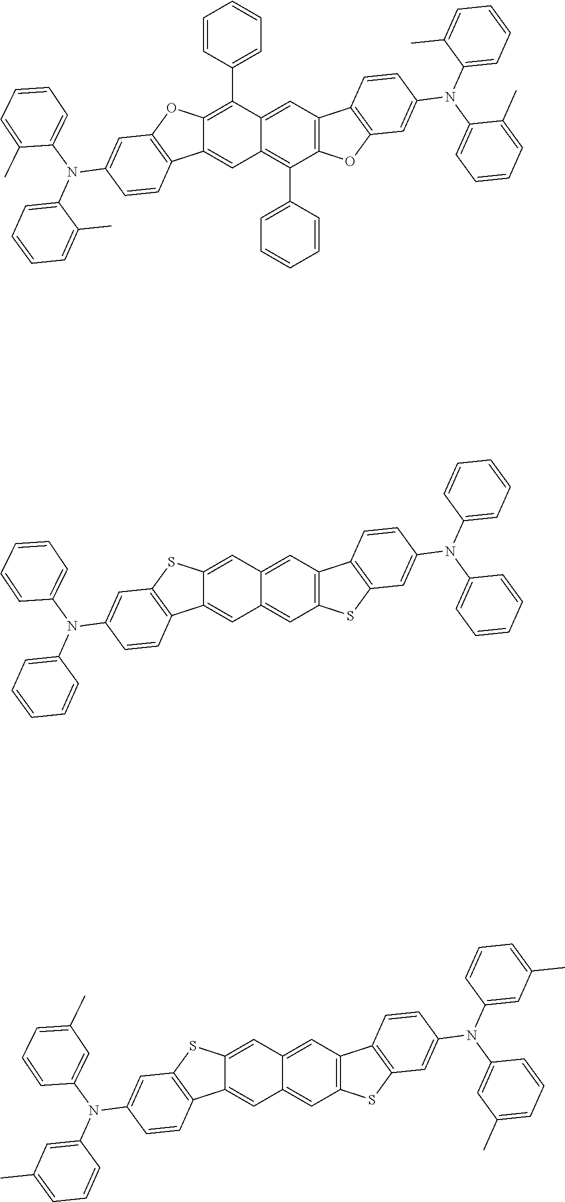

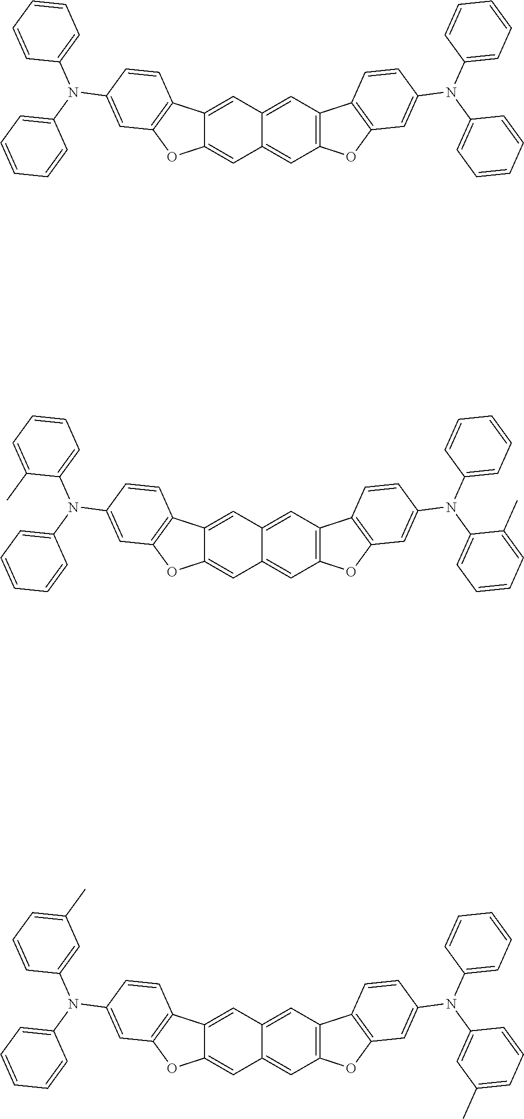 [SMILES: c1ccc(N(c2ccccc2)c2ccc3c(c2)oc2cc4cc5oc6cc(N(c7ccccc7)c7ccccc7)ccc6c5cc4cc23)cc1 ; Cc1ccccc1N(c1ccccc1)c1ccc2c(c1)oc1cc3cc4oc5cc(N(c6ccccc6)c6ccccc6C)ccc5c4cc3cc12 ; Cc1cccc(N(c2ccccc2)c2ccc3c(c2)oc2cc4cc5oc6cc(N(c7ccccc7)c7cccc(C)c7)ccc6c5cc4cc23)c1]

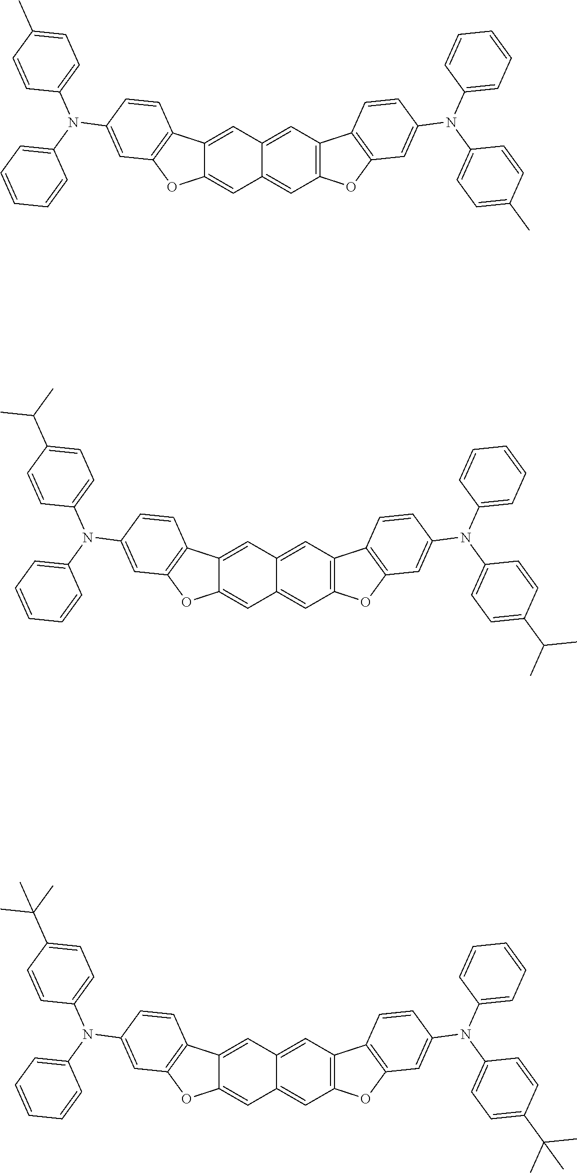

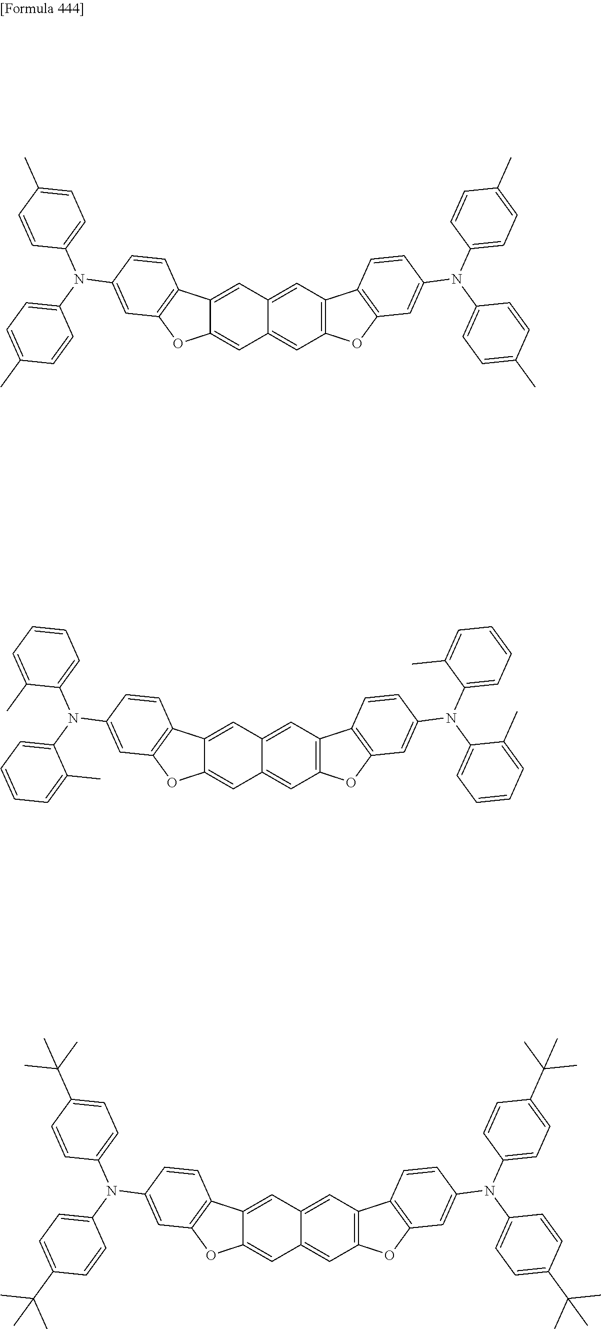

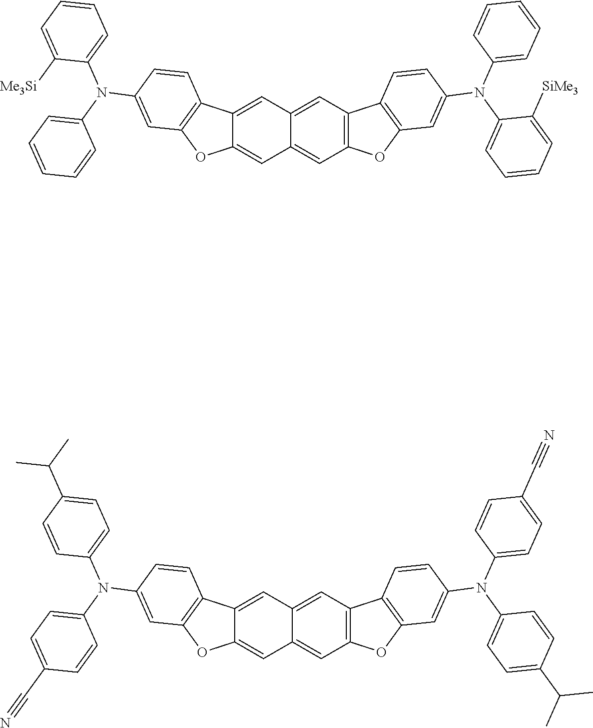 [SMILES: C[Si](C)(C)c1ccccc1N(c1ccccc1)c1ccc2c(c1)oc1cc3cc4oc5cc(N(c6ccccc6)c6ccccc6[Si](C)(C)C)ccc5c4cc3cc12 ; CC(C)c1ccc(N(c2ccc(C#N)cc2)c2ccc3c(c2)oc2cc4cc5oc6cc(N(c7ccc(C#N)cc7)c7ccc(C(C)C)cc7)ccc6c5cc4cc23)cc1]

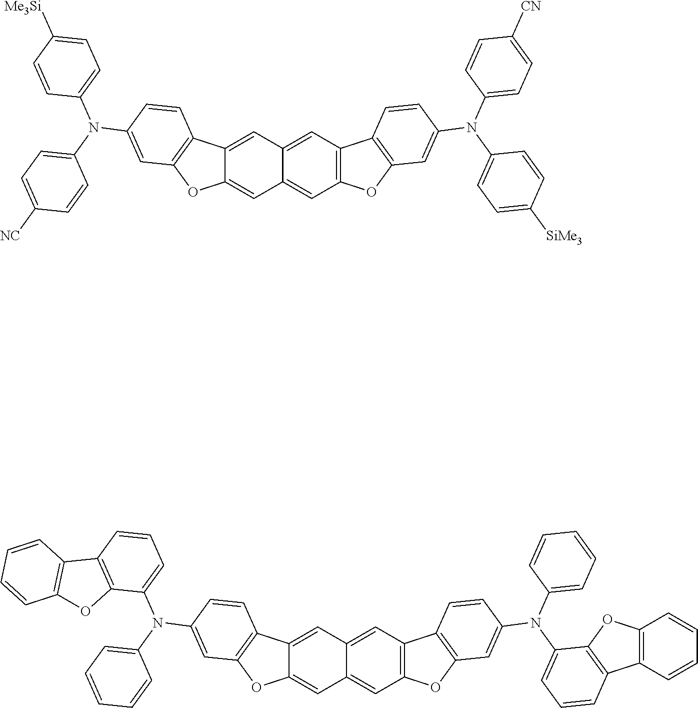 [SMILES: C[Si](C)(C)c1ccc(N(c2ccc(C#N)cc2)c2ccc3c(c2)oc2cc4cc5oc6cc(N(c7ccc(C#N)cc7)c7ccc([Si](C)(C)C)cc7)ccc6c5cc4cc23)cc1 ; c1ccc(N(c2ccc3c(c2)oc2cc4cc5oc6cc(N(c7ccccc7)c7cccc8c7oc7ccccc78)ccc6c5cc4cc23)c2cccc3c2oc2ccccc23)cc1]

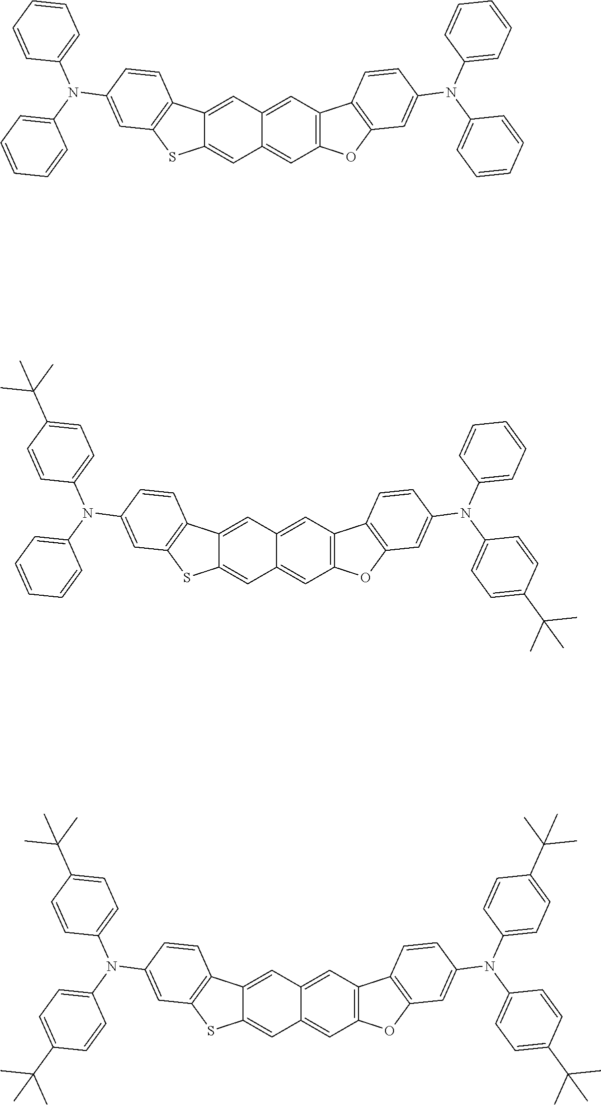

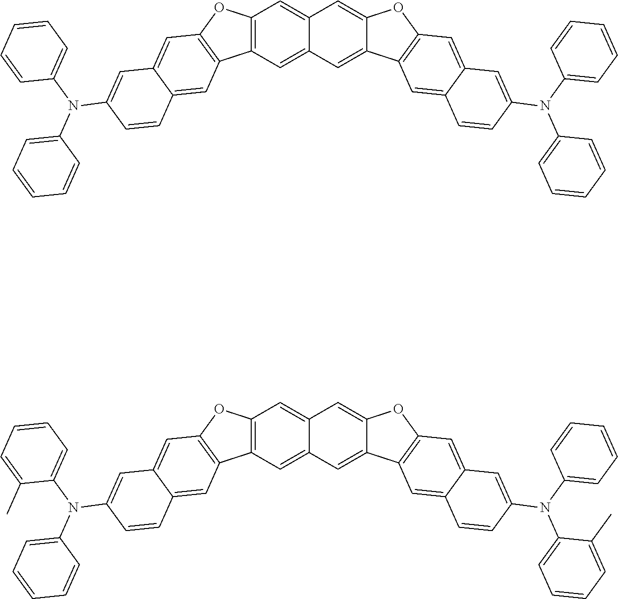 [SMILES: c1ccc(N(c2ccccc2)c2ccc3cc4c(cc3c2)oc2cc3cc5oc6cc7cc(N(c8ccccc8)c8ccccc8)ccc7cc6c5cc3cc24)cc1 ; Cc1ccccc1N(c1ccccc1)c1ccc2cc3c(cc2c1)oc1cc2cc4oc5cc6cc(N(c7ccccc7)c7ccccc7C)ccc6cc5c4cc2cc13]

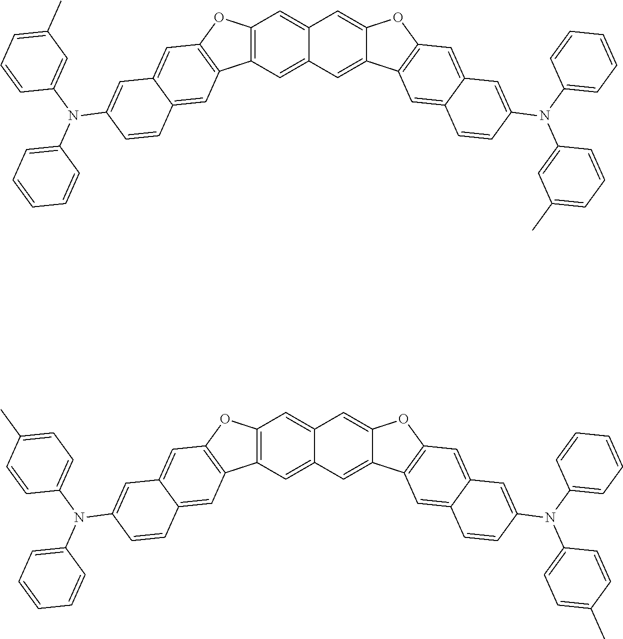 [SMILES: Cc1cccc(N(c2ccccc2)c2ccc3cc4c(cc3c2)oc2cc3cc5oc6cc7cc(N(c8ccccc8)c8cccc(C)c8)ccc7cc6c5cc3cc24)c1 ; Cc1ccc(N(c2ccccc2)c2ccc3cc4c(cc3c2)oc2cc3cc5oc6cc7cc(N(c8ccccc8)c8ccc(C)cc8)ccc7cc6c5cc3cc24)cc1]

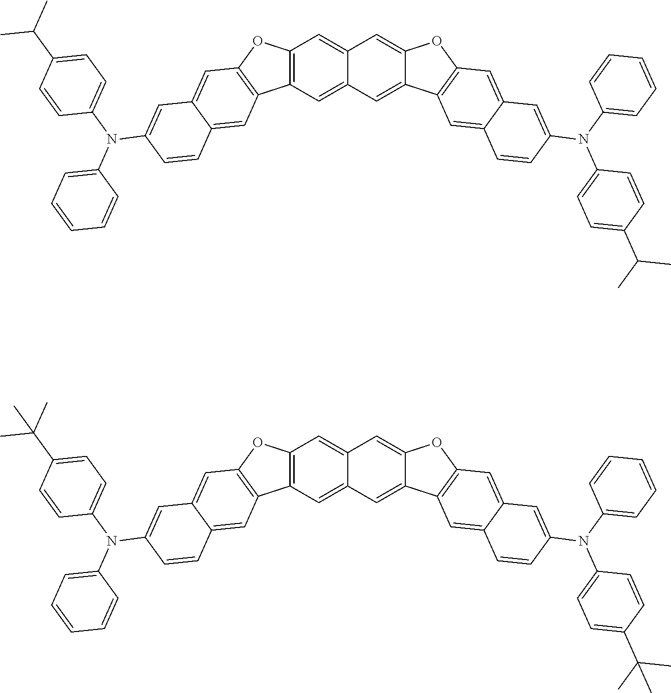

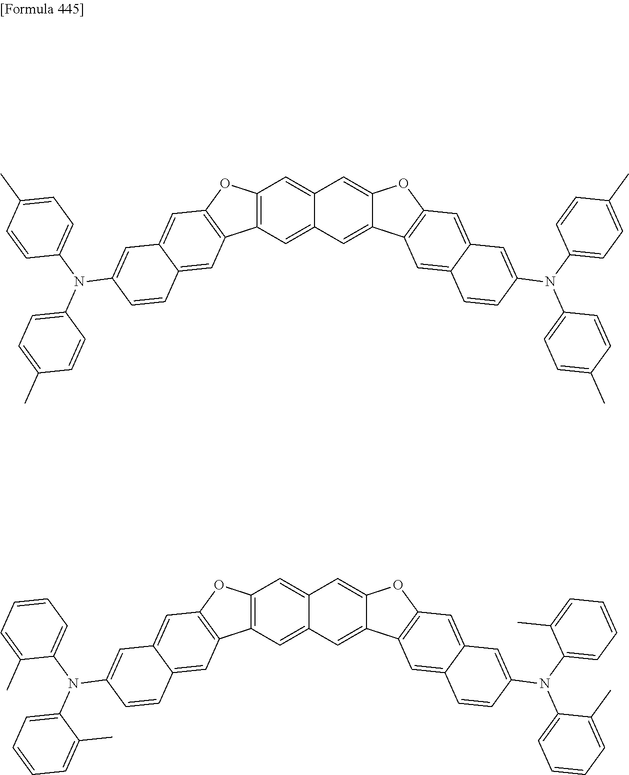

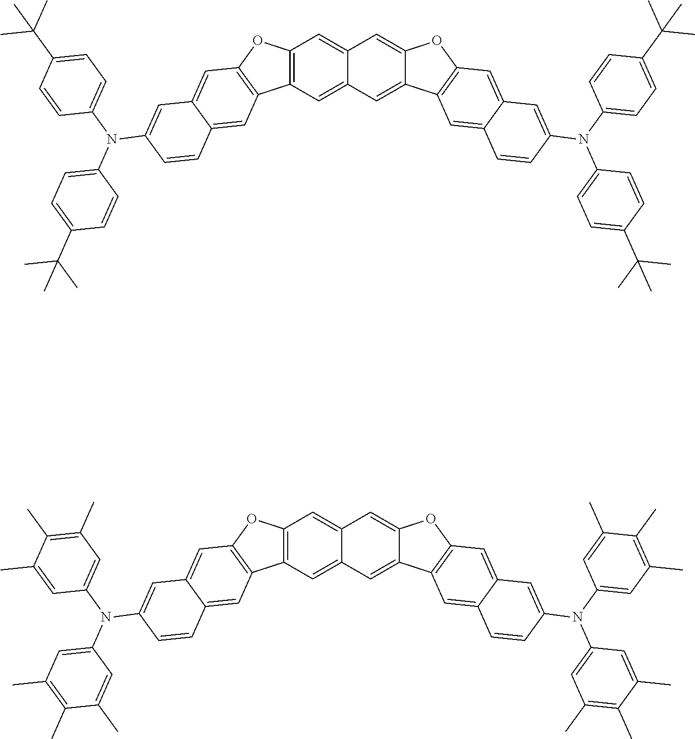

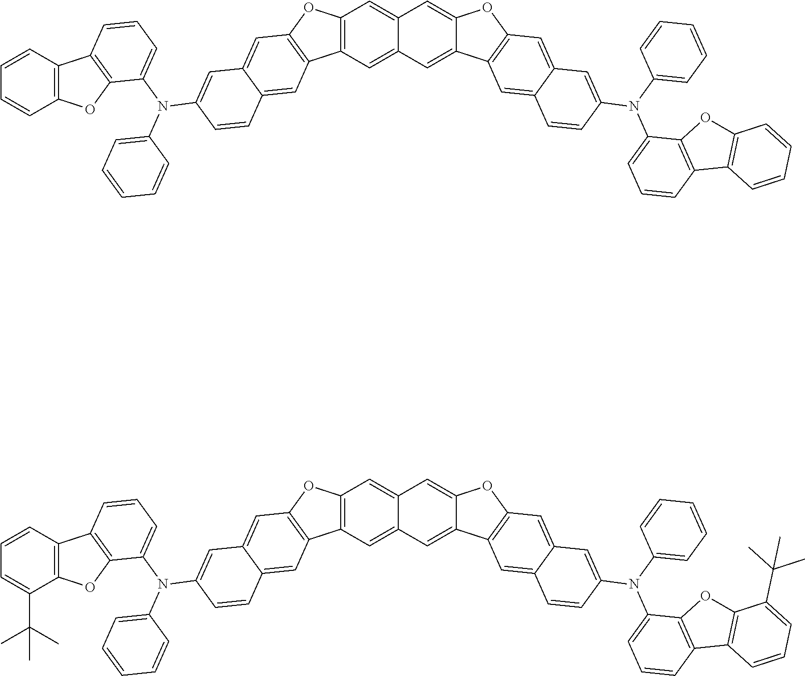 [SMILES: c1ccc(N(c2ccc3cc4c(cc3c2)oc2cc3cc5oc6cc7cc(N(c8ccccc8)c8cccc9c8oc8ccccc89)ccc7cc6c5cc3cc24)c2cccc3c2oc2ccccc23)cc1 ; CC(C)(C)c1cccc2c1oc1c(N(c3ccccc3)c3ccc4cc5c(cc4c3)oc3cc4cc6oc7cc8cc(N(c9ccccc9)c9cccc%10c9oc9c(C(C)(C)C)cccc9%10)ccc8cc7c6cc4cc35)cccc12]

### Compound Represented by Formula (8)

The compound represented by the formula (8) will be described below.

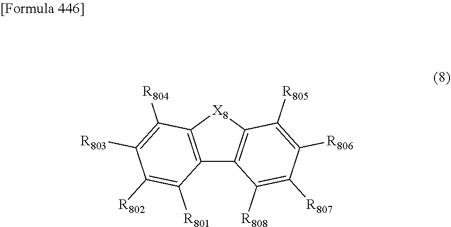

In the formula (8):
at least one of a combination of R₈₀₁ and R₈₀₂, a combination of R₈₀₂ and R₈₀₃, or a combination of R₈₀₃ and R₈₀₄ are mutually bonded to form a divalent group represented by a formula (82) below, and
at least one of a combination of R₈₀₅ and R₈₀₆, a combination of R₈₀₆ and R₈₀₇, or a combination of R₈₀₇ and R₈₀₈ are mutually bonded to form a divalent group represented by a formula (83) below.

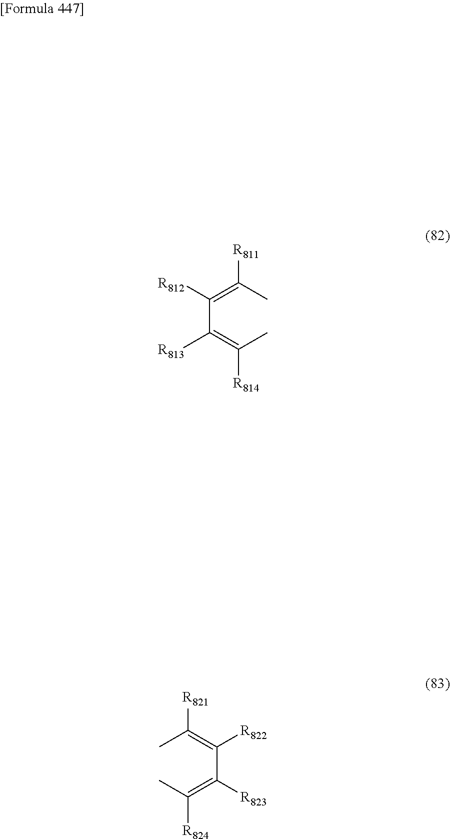

At least one of R₈₀₁ to R₈₀₄ not forming the divalent group represented by the formula (82) or R₈₁₁ to R₈₁₄ is a monovalent group represented by a formula (84) below;
at least one of R₈₀₅ to R₈₀₈ not forming the divalent group represented by the formula (83) or R₈₂₁ to R₈₂₄ is a monovalent group represented by the formula (84) below;
X₈ is an oxygen atom, a sulfur atom, or NR₈₀₉; and
R₈₀₁ to R₈₀₈ not forming the divalent group represented by the formula (82) or (83) and not being the monovalent group represented by the formula (84), R₈₁₁ to R₈₁₄ and R₈₂₁ to R₈₂₄ not being the monovalent group represented by the formula (84), and R₈₀₉ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

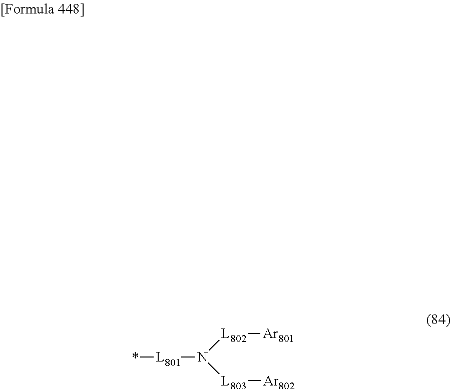

In the formula (84):
Ar₈₀₁ and Ar₈₀₂ are each independently a substituted or unsubstituted aryl group having 6 to 50 ring carbon atoms, or a substituted or unsubstituted heterocyclic group having 5 to 50 ring atoms;
L₈₀₁ to L₈₀₃ are each independently a single bond, a substituted or unsubstituted arylene group having 6 to 30 ring carbon atoms, a substituted or unsubstituted divalent heterocyclic group having 5 to 30 ring atoms, or a divalent linking group formed by bonding two, three or four groups selected from the group consisting of a substituted or unsubstituted arylene group having 6 to 30 ring carbon atoms and a substituted or unsubstituted divalent heterocyclic group having 5 to 30 ring atoms; and
* in the formula (84) represents a bonding position to a cyclic structure represented by the formula (8) or a bonding position to a group represented by the formula (82) or (83).

In the formula (8), the positions for the divalent group represented by the formula (82) and the divalent group represented by the formula (83) to be formed are not specifically limited but the divalent groups may be formed at any possible positions on R₈₀₁ to R₈₀₈.

In an exemplary embodiment, the compound represented by the formula (8) is represented by any one of formulae (81-1) to (81-6) below.

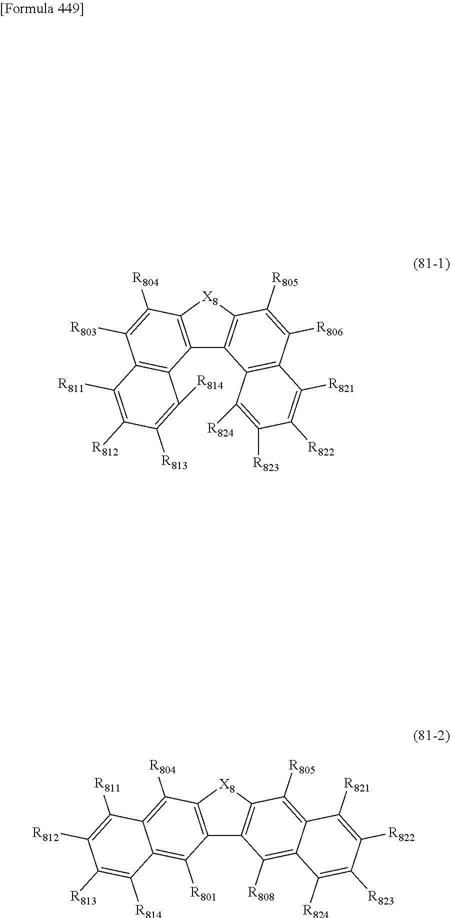

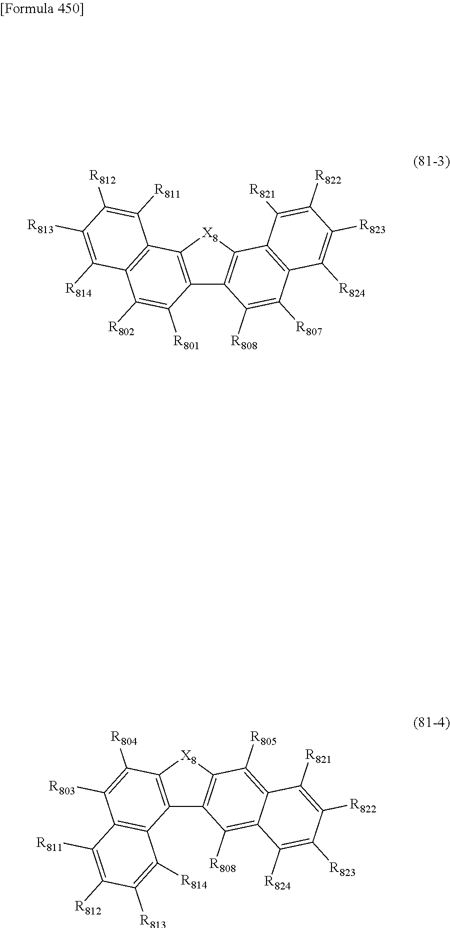

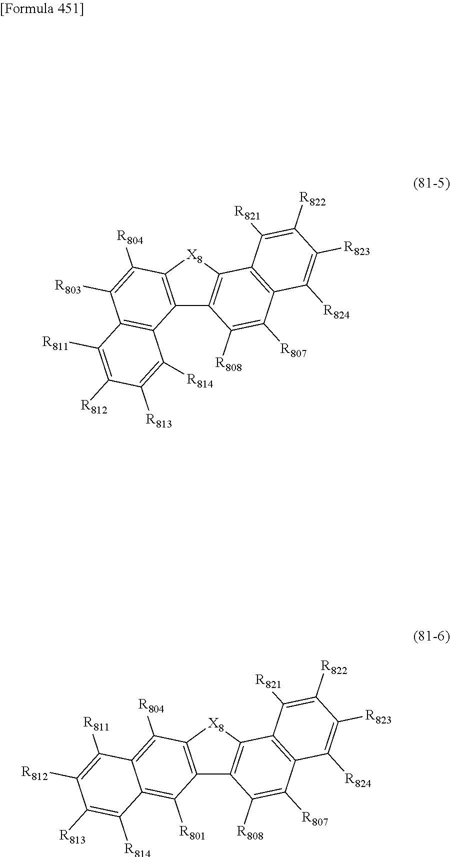

In the formulae (81-1) to (81-6):
X₈ represents the same as X₈ in the formula (8);
at least two of R₈₀₁ to R₈₂₄ are each a monovalent group represented by the formula (84); and
R₈₀₁ to R₈₂₄ not being the monovalent group represented by the formula (84) are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In an exemplary embodiment, the compound represented by the formula (8) is represented by any one of formulae (81-7) to (81-18) below.

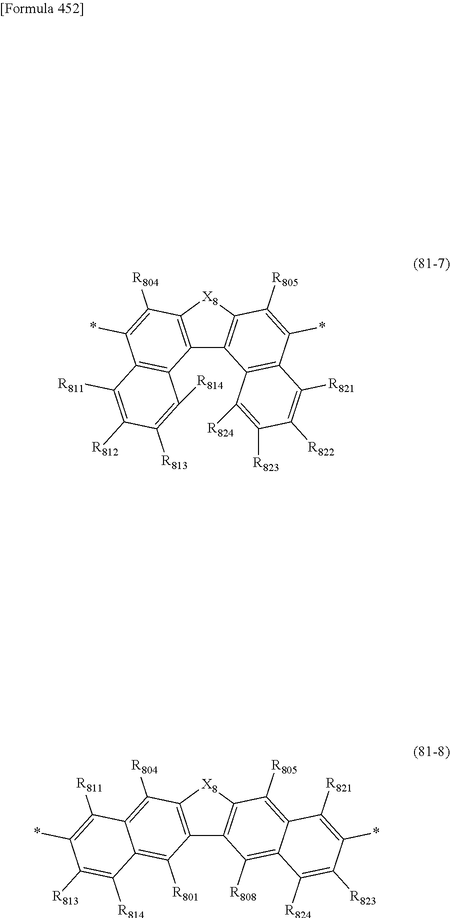

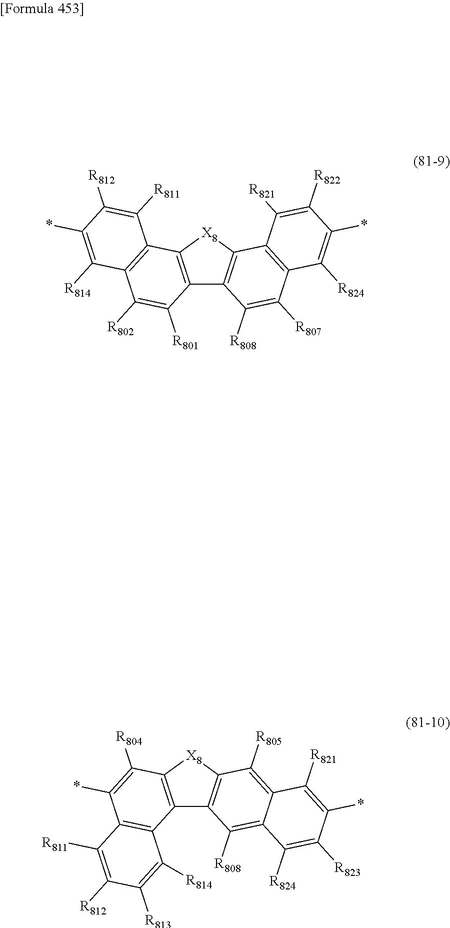

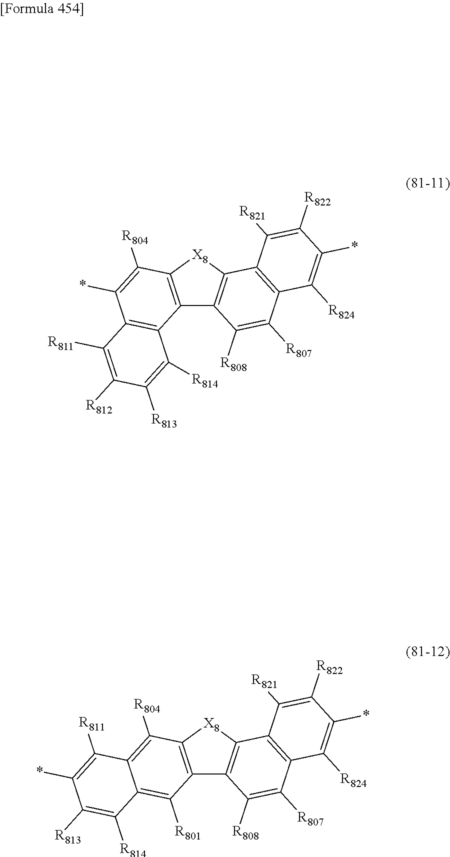

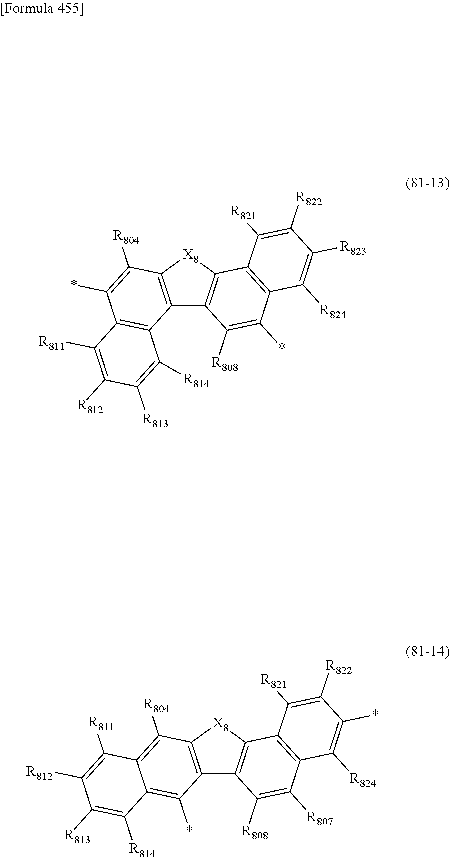

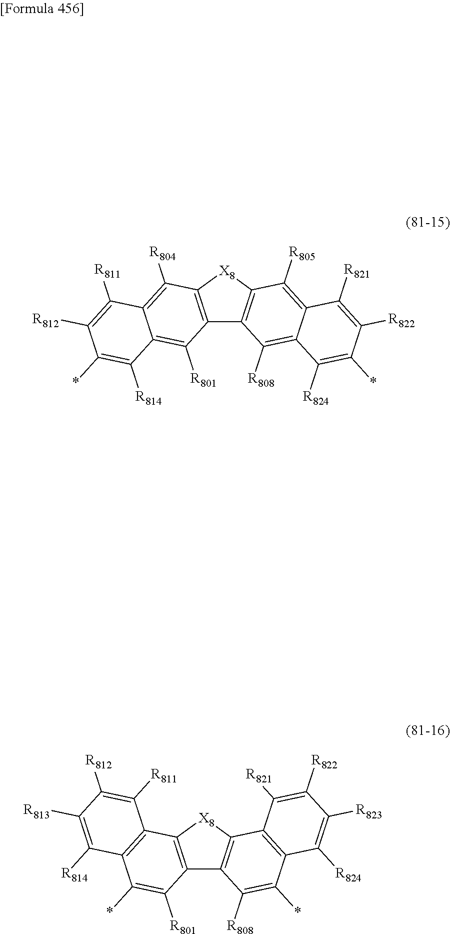

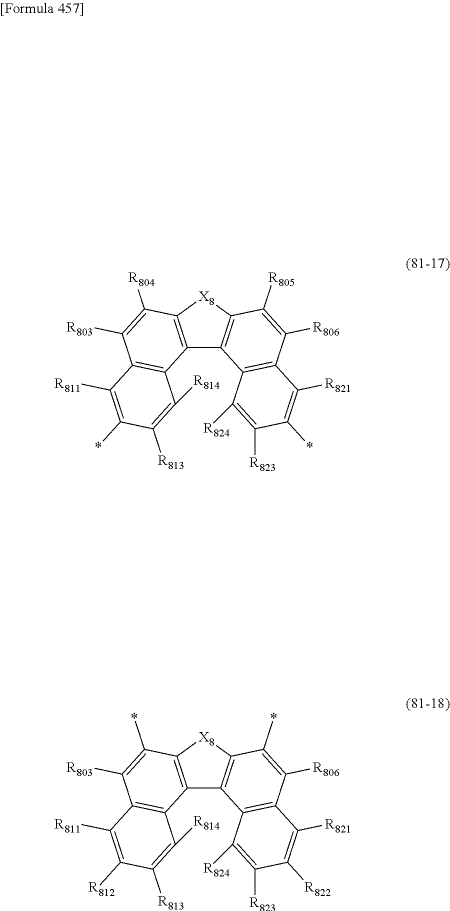

In the formulae (81-7) to (81-18):
X₈ represents the same as X₈ in the formula (8);
* are each a single bond bonded to a monovalent group represented by the formula (84); and
R₈₀₁ to R₈₂₄ each independently represent the same as R₈₀₁ to R₈₂₄ in the formulae (81-1) to (81-6) not being the monovalent group represented by the formula (84).

R₈₀₁ to R₈₀₈ not forming the divalent group represented by the formula (82) or (83) and not being the monovalent group represented by the formula (84), and R₈₁₁ to R₈₁₄ and R₈₂₁ to R₈₂₄ not being the monovalent group represented by the formula (84) are preferably each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

The monovalent group represented by the formula (84) is preferably represented by a formula (85) or (86) below.

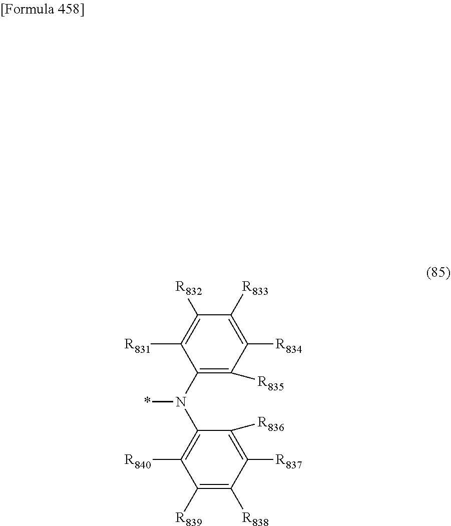

In the formula (85):
R₈₃₁ to R₈₄₀ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms; and
* in the formula (85) represents the same as * in the formula (84).

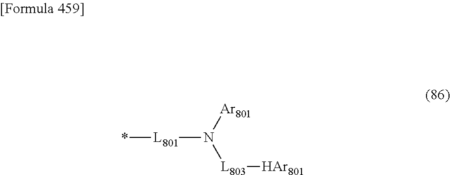

In the formula (86):
Ar₈₀₁, L₈₀₁, and L₈₀₃ represent the same as Ar₈₀₁, L₈₀₁, and L₈₀₃ in the formula (84); and
HAr₈₀₁ is a structure represented by a formula (87) below.

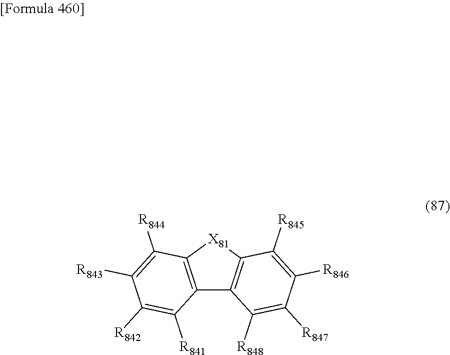

In the formula (87):
X₈₁ is an oxygen atom or a sulfur atom;
one of R₈₄₁ to R₈₄₈ is a single bond with L₈₀₃; and
R₈₄₁ to R₈₄₈ not being the single bond are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

### Specific Examples of Compound Represented by Formula (8)

Specific examples of the compound represented by the formula (8) include compounds given below as well as the compounds disclosed in WO 2014/104144.

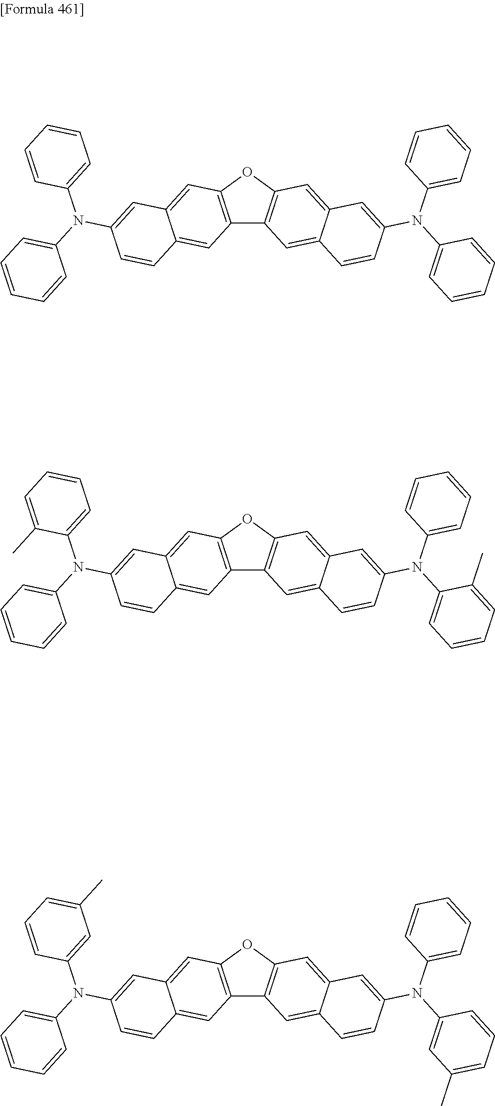 [SMILES: c1ccc(N(c2ccccc2)c2ccc3cc4c(cc3c2)oc2cc3cc(N(c5ccccc5)c5ccccc5)ccc3cc24)cc1 ; Cc1ccccc1N(c1ccccc1)c1ccc2cc3c(cc2c1)oc1cc2cc(N(c4ccccc4)c4ccccc4C)ccc2cc13 ; Cc1cccc(N(c2ccccc2)c2ccc3cc4c(cc3c2)oc2cc3cc(N(c5ccccc5)c5cccc(C)c5)ccc3cc24)c1]

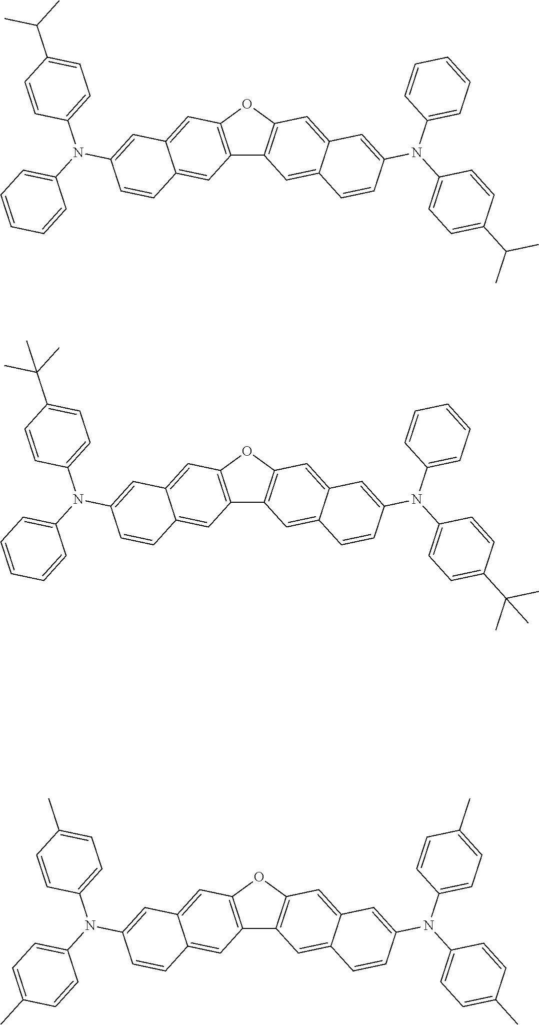

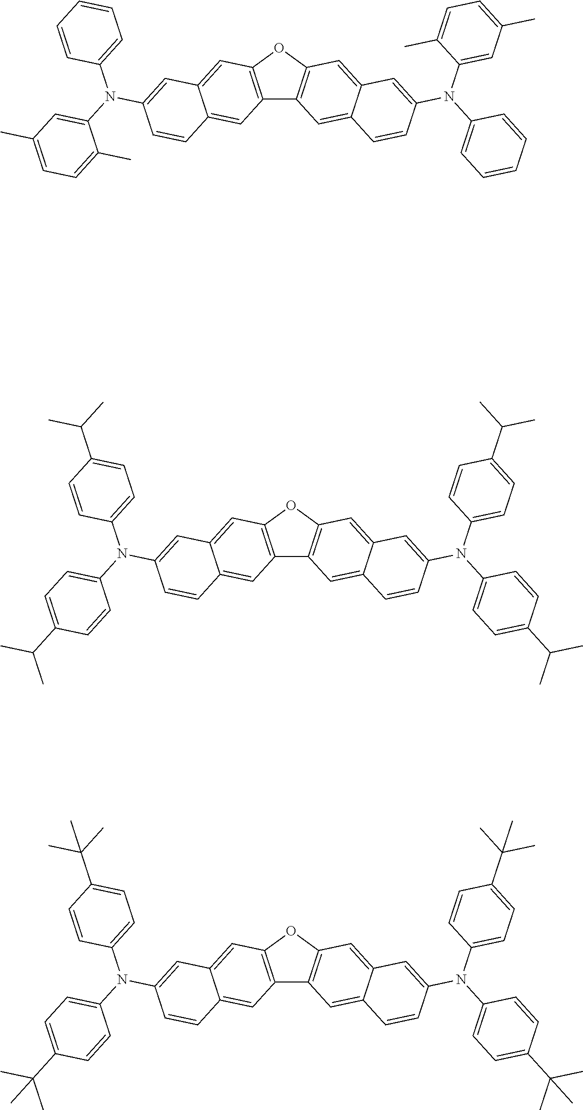

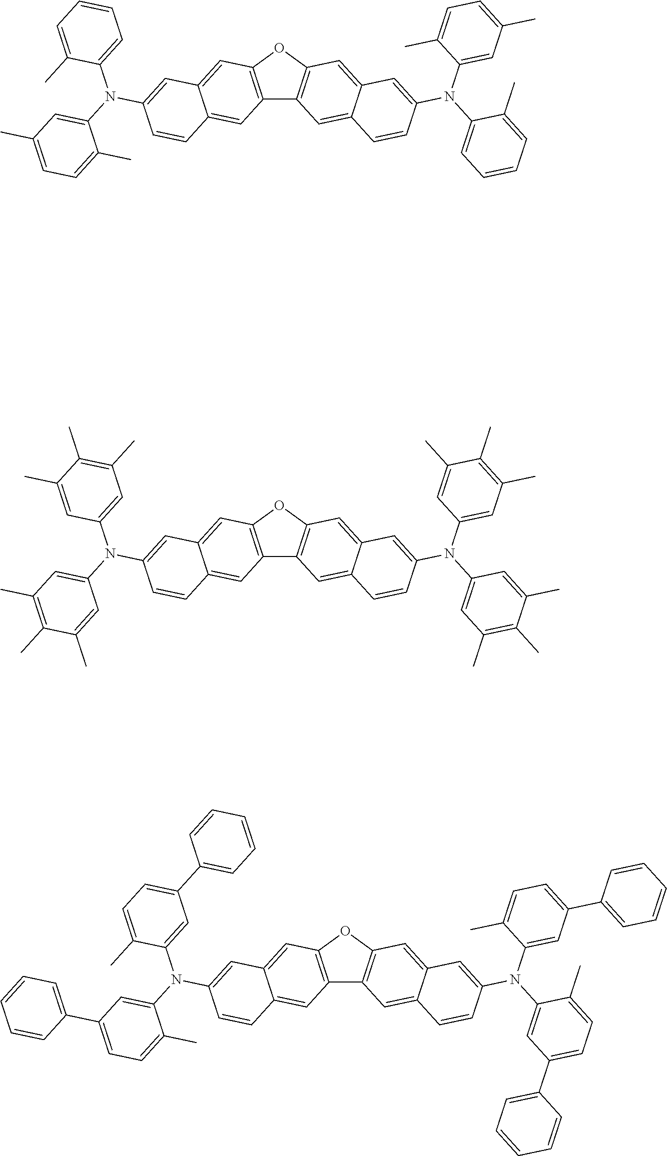

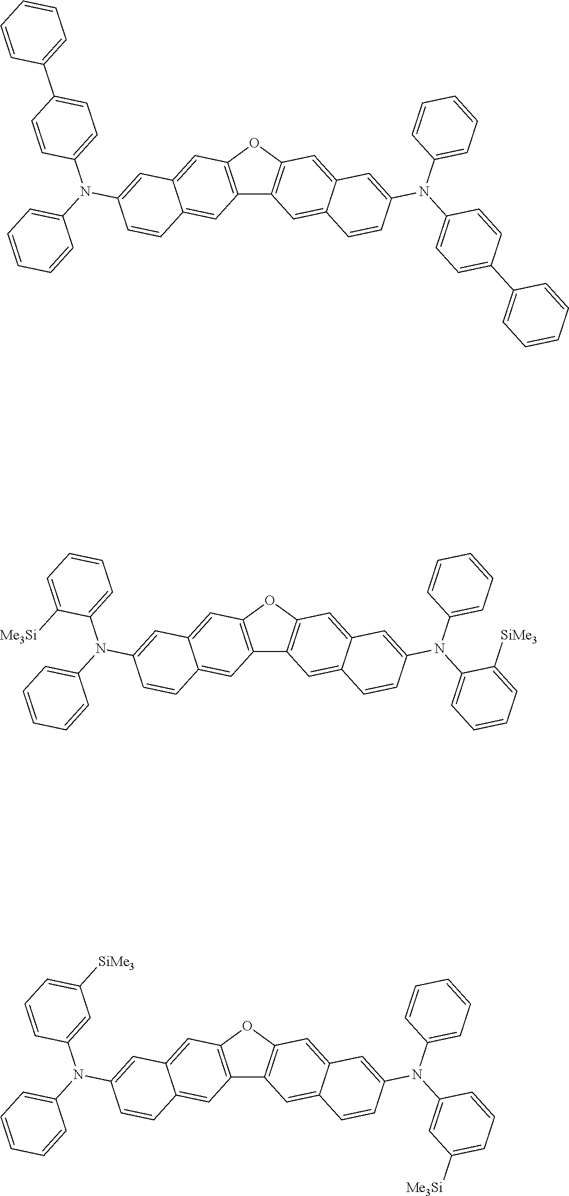 [SMILES: c1ccc(-c2ccc(N(c3ccccc3)c3ccc4cc5c(cc4c3)oc3cc4cc(N(c6ccccc6)c6ccc(-c7ccccc7)cc6)ccc4cc35)cc2)cc1 ; C[Si](C)(C)c1ccccc1N(c1ccccc1)c1ccc2cc3c(cc2c1)oc1cc2cc(N(c4ccccc4)c4ccccc4[Si](C)(C)C)ccc2cc13 ; C[Si](C)(C)c1cccc(N(c2ccccc2)c2ccc3cc4c(cc3c2)oc2cc3cc(N(c5ccccc5)c5cccc([Si](C)(C)C)c5)ccc3cc24)c1]

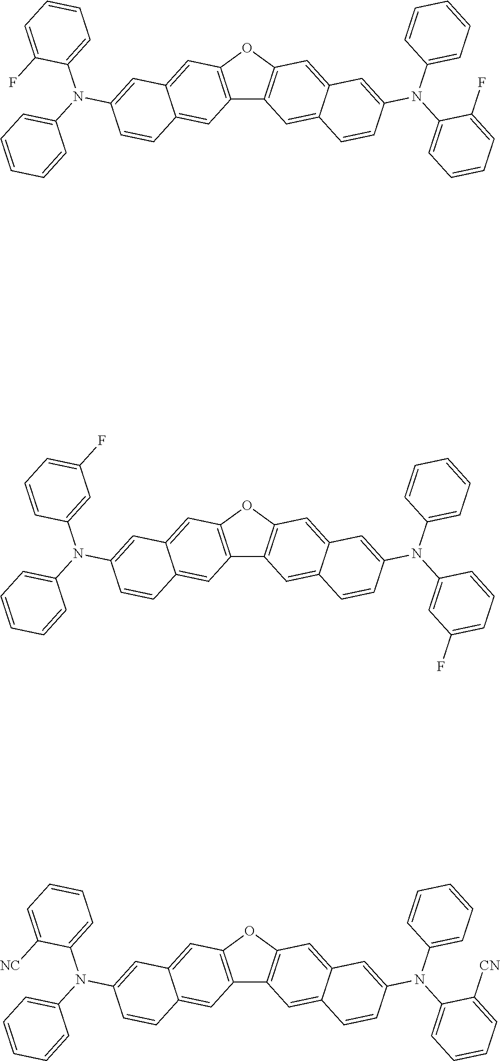 [SMILES: Fc1ccccc1N(c1ccccc1)c1ccc2cc3c(cc2c1)oc1cc2cc(N(c4ccccc4)c4ccccc4F)ccc2cc13 ; Fc1cccc(N(c2ccccc2)c2ccc3cc4c(cc3c2)oc2cc3cc(N(c5ccccc5)c5cccc(F)c5)ccc3cc24)c1 ; N#Cc1ccccc1N(c1ccccc1)c1ccc2cc3c(cc2c1)oc1cc2cc(N(c4ccccc4)c4ccccc4C#N)ccc2cc13]

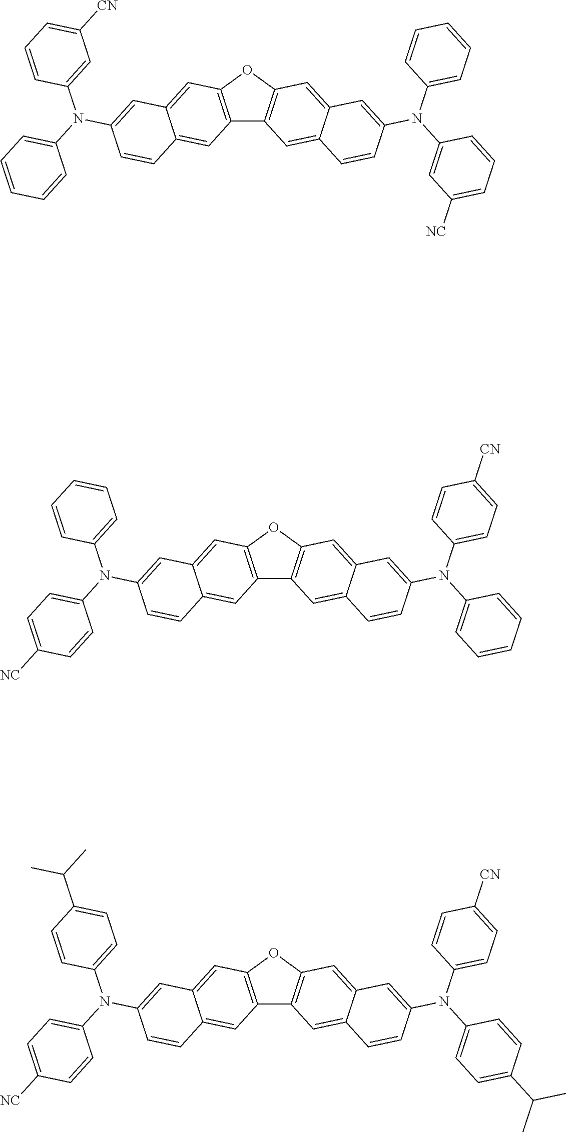 [SMILES: N#Cc1cccc(N(c2ccccc2)c2ccc3cc4c(cc3c2)oc2cc3cc(N(c5ccccc5)c5cccc(C#N)c5)ccc3cc24)c1 ; N#Cc1ccc(N(c2ccccc2)c2ccc3cc4c(cc3c2)oc2cc3cc(N(c5ccccc5)c5ccc(C#N)cc5)ccc3cc24)cc1 ; CC(C)c1ccc(N(c2ccc(C#N)cc2)c2ccc3cc4c(cc3c2)oc2cc3cc(N(c5ccc(C#N)cc5)c5ccc(C(C)C)cc5)ccc3cc24)cc1]

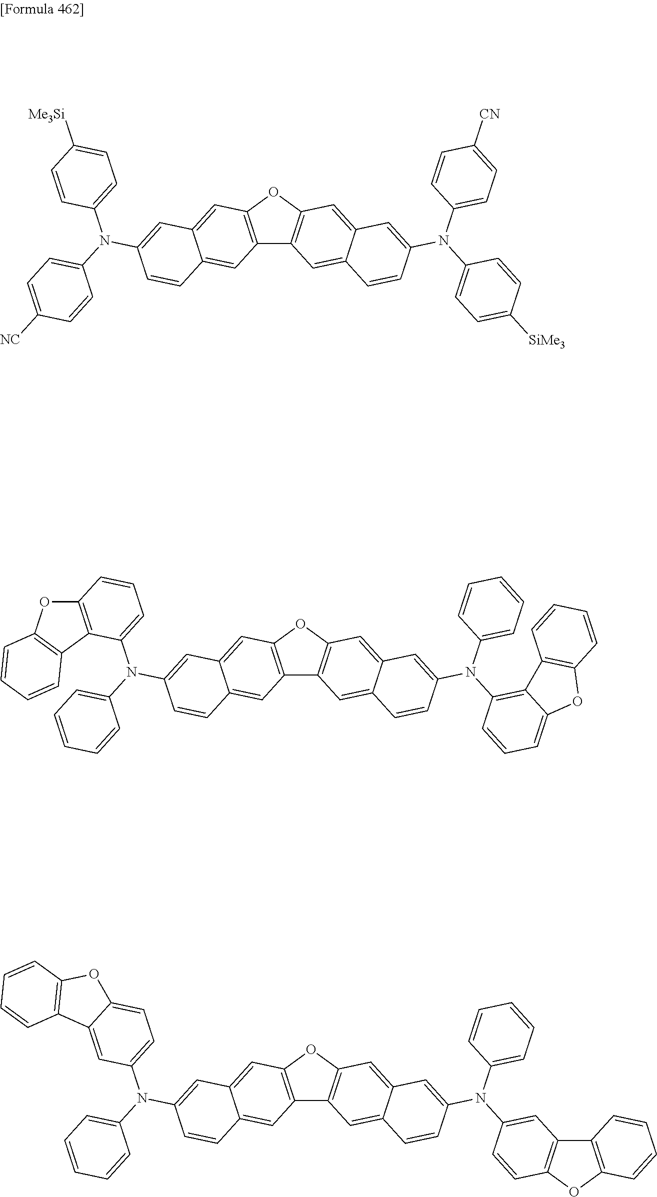 [SMILES: C[Si](C)(C)c1ccc(N(c2ccc(C#N)cc2)c2ccc3cc4c(cc3c2)oc2cc3cc(N(c5ccc(C#N)cc5)c5ccc([Si](C)(C)C)cc5)ccc3cc24)cc1 ; c1ccc(N(c2ccc3cc4c(cc3c2)oc2cc3cc(N(c5ccccc5)c5cccc6oc7ccccc7c56)ccc3cc24)c2cccc3oc4ccccc4c23)cc1 ; c1ccc(N(c2ccc3cc4c(cc3c2)oc2cc3cc(N(c5ccccc5)c5ccc6oc7ccccc7c6c5)ccc3cc24)c2ccc3oc4ccccc4c3c2)cc1]

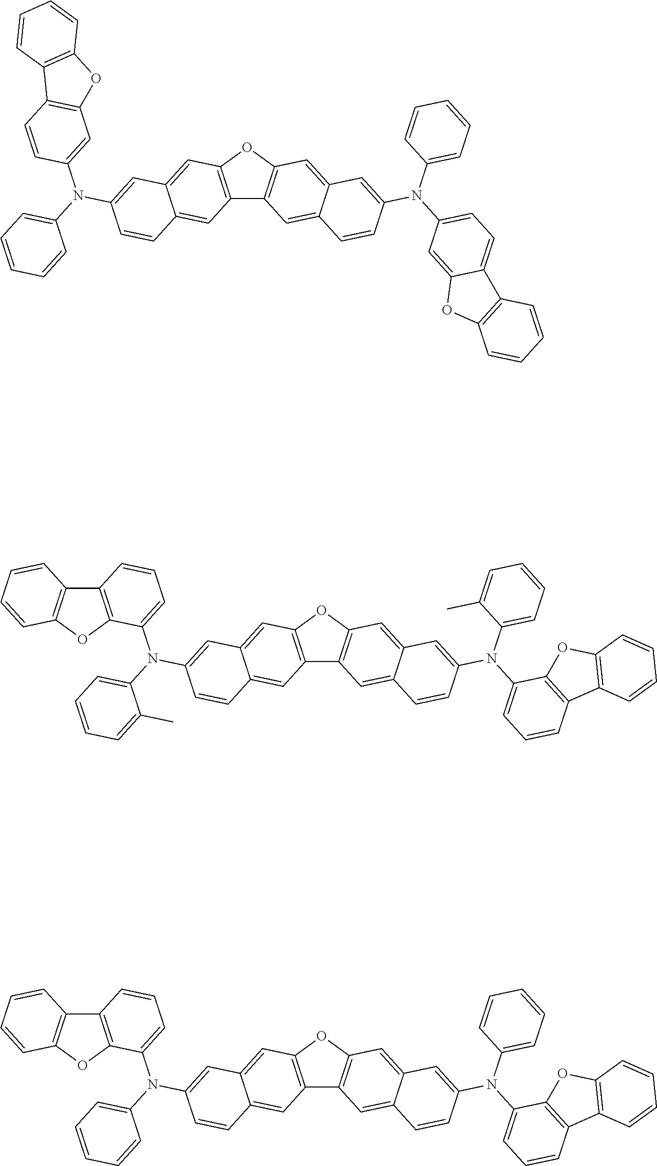 [SMILES: c1ccc(N(c2ccc3cc4c(cc3c2)oc2cc3cc(N(c5ccccc5)c5ccc6c(c5)oc5ccccc56)ccc3cc24)c2ccc3c(c2)oc2ccccc23)cc1 ; Cc1ccccc1N(c1ccc2cc3c(cc2c1)oc1cc2cc(N(c4ccccc4C)c4cccc5c4oc4ccccc45)ccc2cc13)c1cccc2c1oc1ccccc12 ; c1ccc(N(c2ccc3cc4c(cc3c2)oc2cc3cc(N(c5ccccc5)c5cccc6c5oc5ccccc56)ccc3cc24)c2cccc3c2oc2ccccc23)cc1]

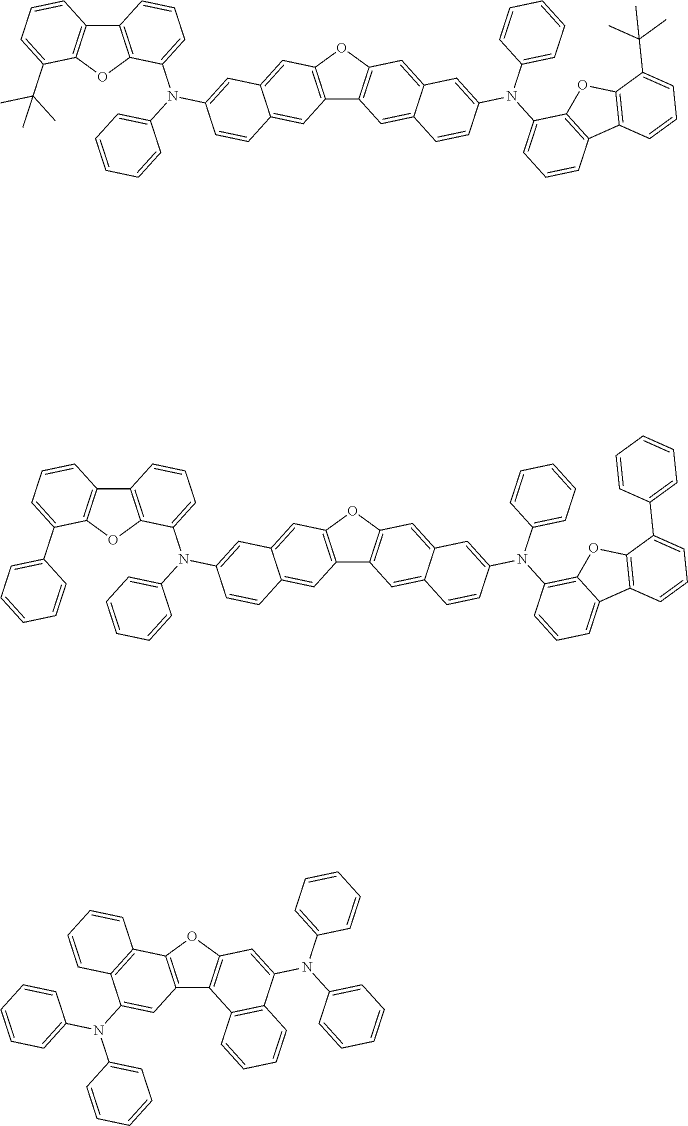 [SMILES: CC(C)(C)c1cccc2c1oc1c(N(c3ccccc3)c3ccc4cc5c(cc4c3)oc3cc4cc(N(c6ccccc6)c6cccc7c6oc6c(C(C)(C)C)cccc67)ccc4cc35)cccc12 ; c1ccc(-c2cccc3c2oc2c(N(c4ccccc4)c4ccc5cc6c(cc5c4)oc4cc5cc(N(c7ccccc7)c7cccc8c7oc7c(-c9ccccc9)cccc78)ccc5cc46)cccc23)cc1 ; c1ccc(N(c2ccccc2)c2cc3c(oc4cc(N(c5ccccc5)c5ccccc5)c5ccccc5c43)c3ccccc23)cc1]

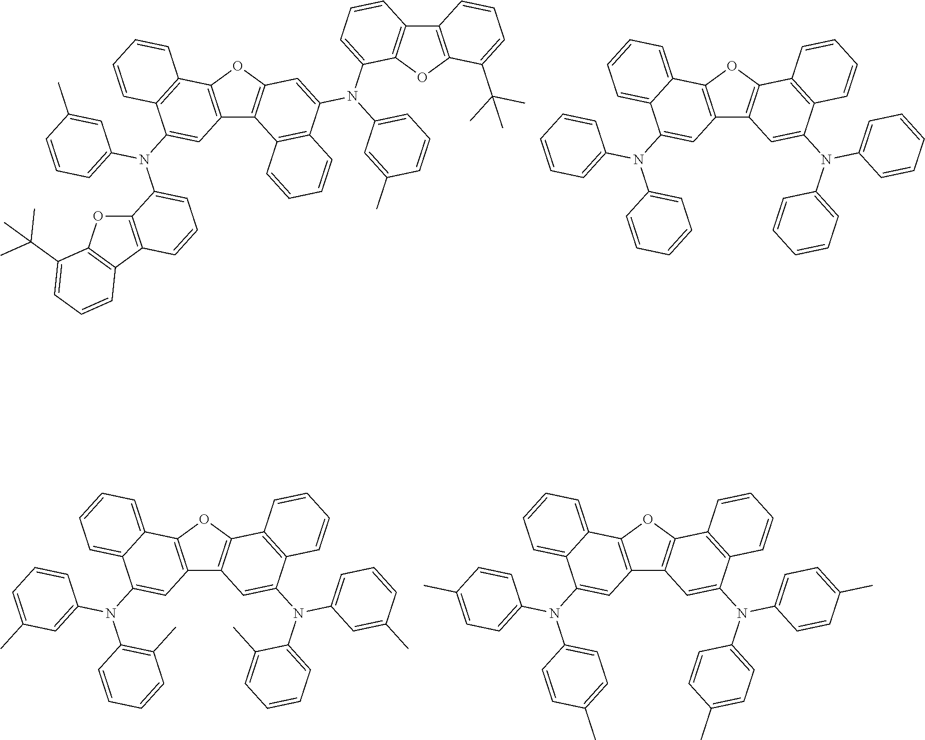

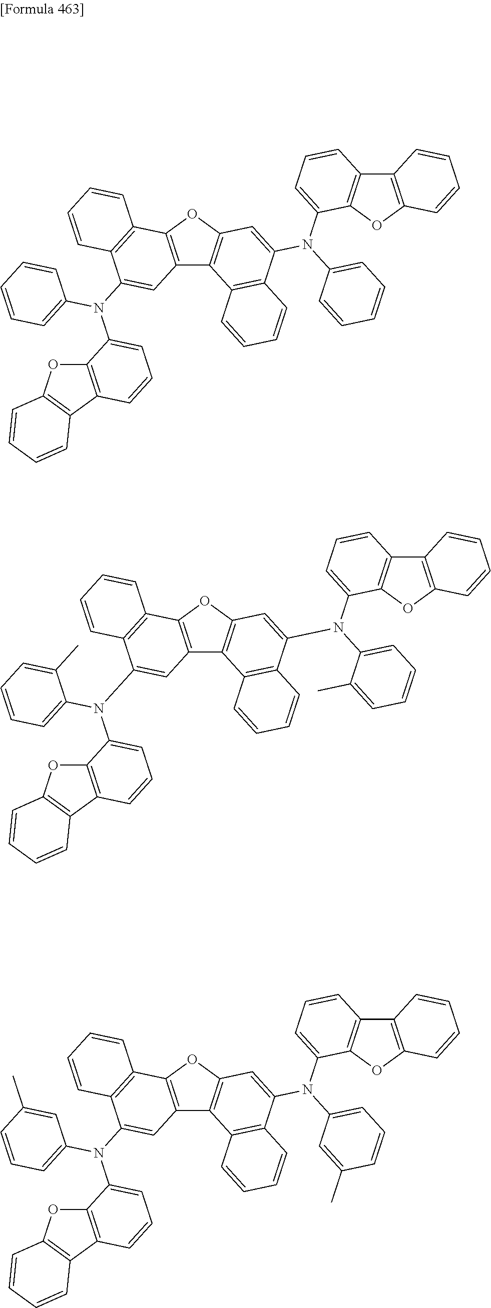 [SMILES: c1ccc(N(c2cc3c(oc4cc(N(c5ccccc5)c5cccc6c5oc5ccccc56)c5ccccc5c43)c3ccccc23)c2cccc3c2oc2ccccc23)cc1 ; Cc1ccccc1N(c1cc2c(oc3cc(N(c4ccccc4C)c4cccc5c4oc4ccccc45)c4ccccc4c32)c2ccccc12)c1cccc2c1oc1ccccc12 ; Cc1cccc(N(c2cc3c(oc4cc(N(c5cccc(C)c5)c5cccc6c5oc5ccccc56)c5ccccc5c43)c3ccccc23)c2cccc3c2oc2ccccc23)c1]

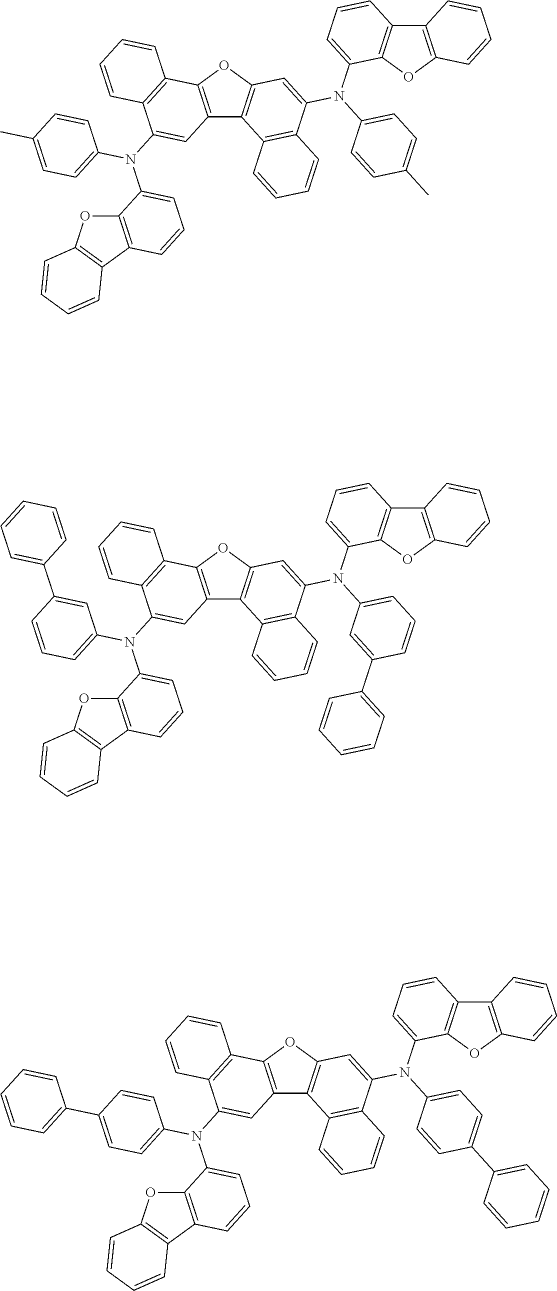 [SMILES: Cc1ccc(N(c2cc3c(oc4cc(N(c5ccc(C)cc5)c5cccc6c5oc5ccccc56)c5ccccc5c43)c3ccccc23)c2cccc3c2oc2ccccc23)cc1 ; c1ccc(-c2cccc(N(c3cc4c(oc5cc(N(c6cccc(-c7ccccc7)c6)c6cccc7c6oc6ccccc67)c6ccccc6c54)c4ccccc34)c3cccc4c3oc3ccccc34)c2)cc1 ; c1ccc(-c2ccc(N(c3cc4c(oc5cc(N(c6ccc(-c7ccccc7)cc6)c6cccc7c6oc6ccccc67)c6ccccc6c54)c4ccccc34)c3cccc4c3oc3ccccc34)cc2)cc1]

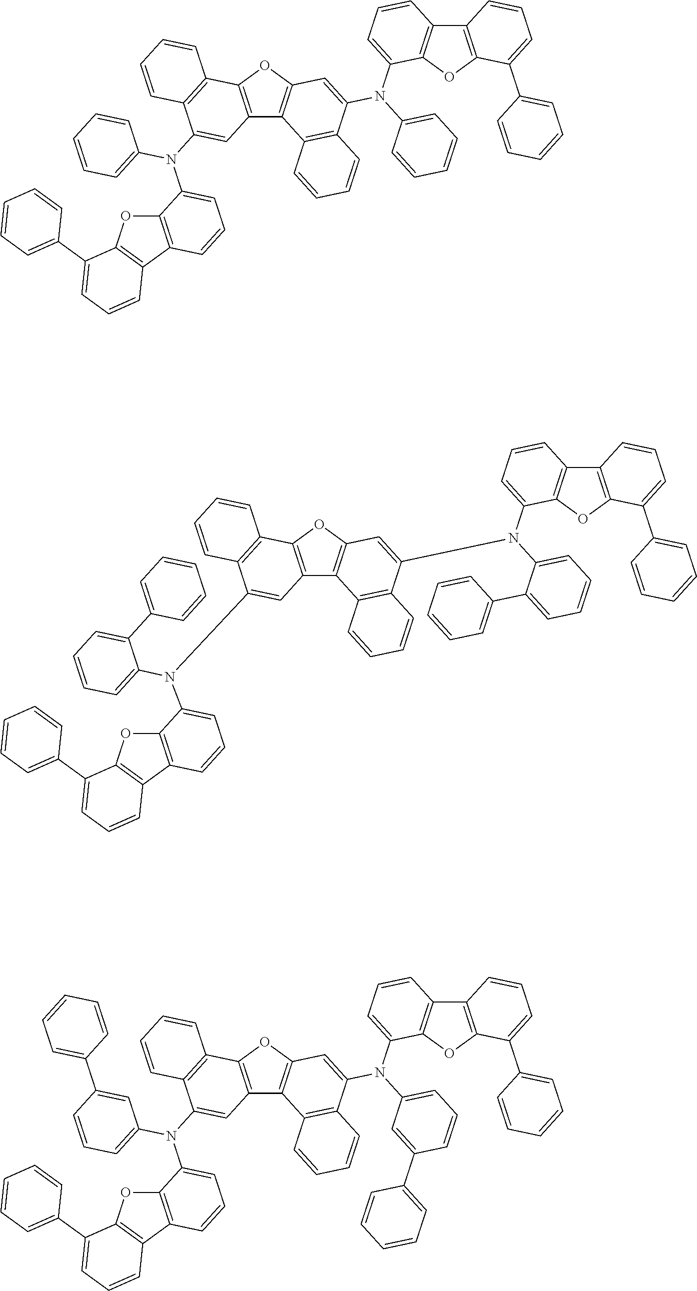 [SMILES: c1ccc(-c2cccc3c2oc2c(N(c4ccccc4)c4cc5c(oc6cc(N(c7ccccc7)c7cccc8c7oc7c(-c9ccccc9)cccc78)c7ccccc7c65)c5ccccc45)cccc23)cc1 ; c1ccc(-c2ccccc2N(c2cc3c(oc4cc(N(c5ccccc5-c5ccccc5)c5cccc6c5oc5c(-c7ccccc7)cccc56)c5ccccc5c43)c3ccccc23)c2cccc3c2oc2c(-c4ccccc4)cccc23)cc1 ; c1ccc(-c2cccc(N(c3cc4c(oc5cc(N(c6cccc(-c7ccccc7)c6)c6cccc7c6oc6c(-c8ccccc8)cccc67)c6ccccc6c54)c4ccccc34)c3cccc4c3oc3c(-c5ccccc5)cccc34)c2)cc1]

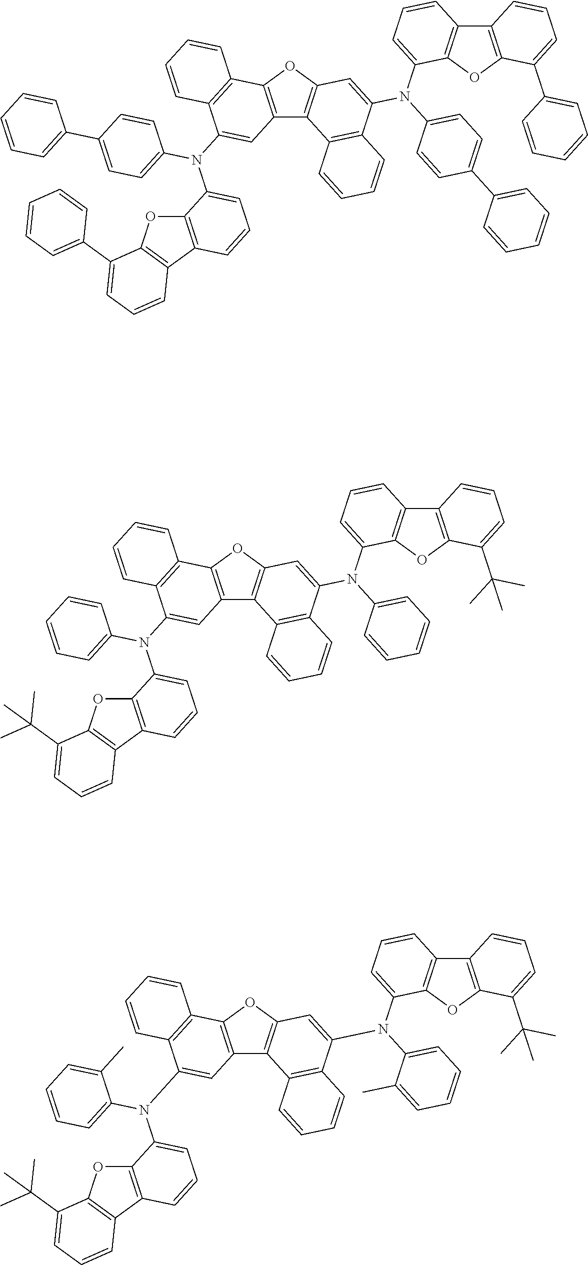

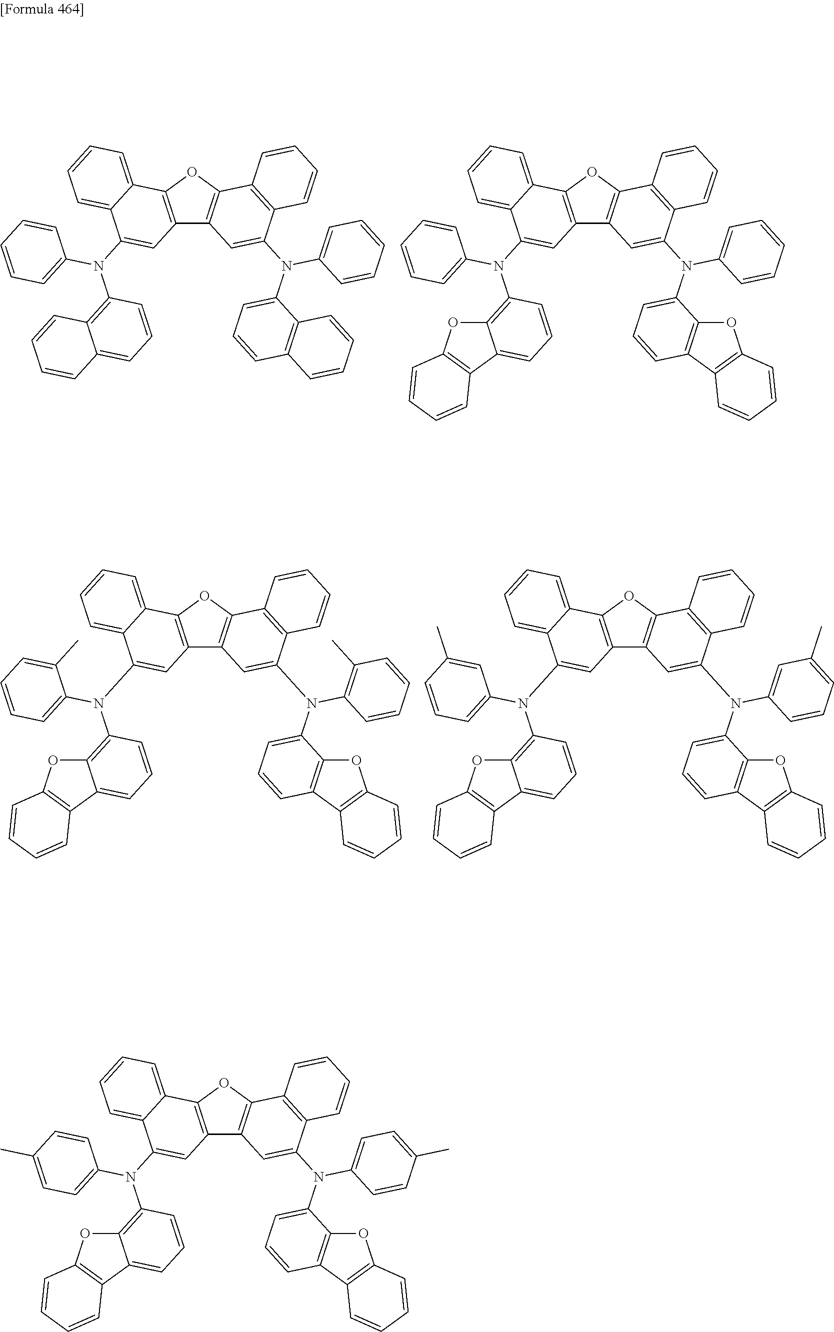

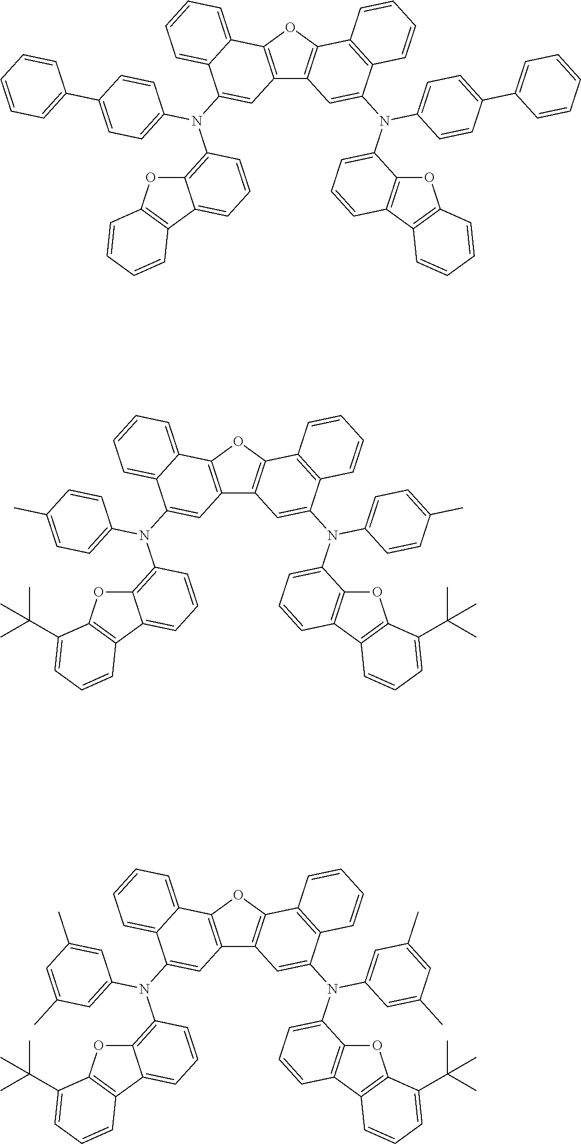

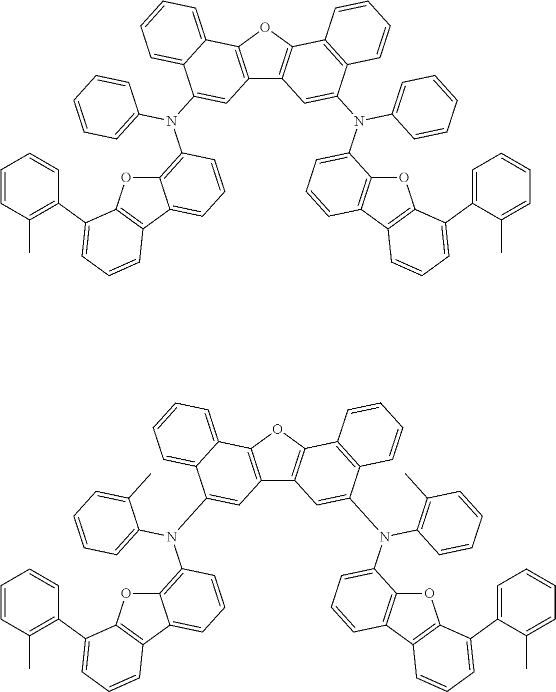 [SMILES: Cc1ccccc1-c1cccc2c1oc1c(N(c3ccccc3)c3cc4c5cc(N(c6ccccc6)c6cccc7c6oc6c(-c8ccccc8C)cccc67)c6ccccc6c5oc4c4ccccc34)cccc12 ; Cc1ccccc1-c1cccc2c1oc1c(N(c3ccccc3C)c3cc4c5cc(N(c6ccccc6C)c6cccc7c6oc6c(-c8ccccc8C)cccc67)c6ccccc6c5oc4c4ccccc34)cccc12]

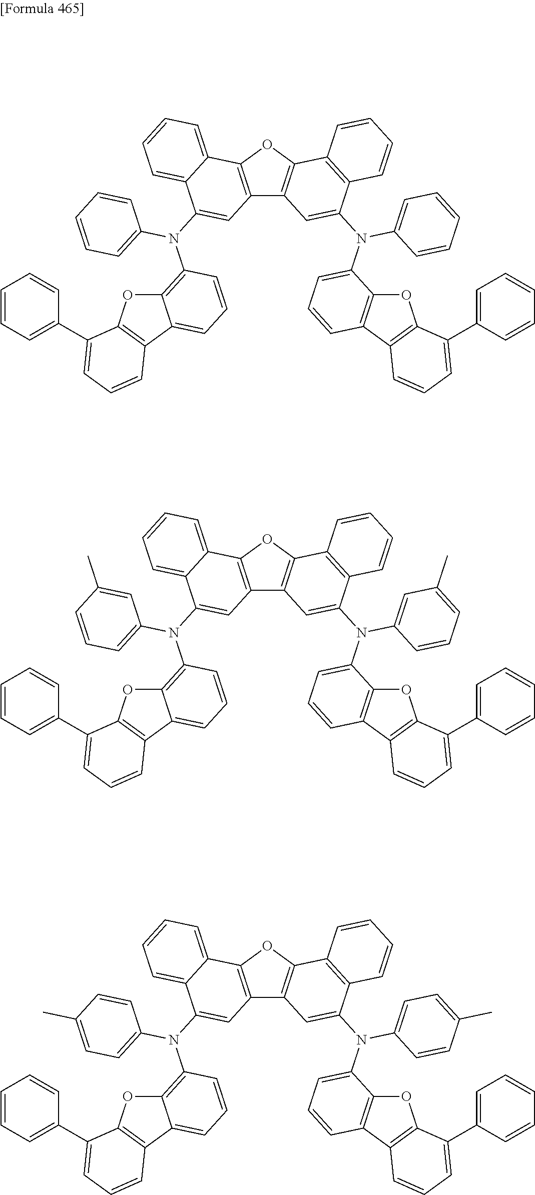 [SMILES: c1ccc(-c2cccc3c2oc2c(N(c4ccccc4)c4cc5c6cc(N(c7ccccc7)c7cccc8c7oc7c(-c9ccccc9)cccc78)c7ccccc7c6oc5c5ccccc45)cccc23)cc1 ; Cc1cccc(N(c2cc3c4cc(N(c5cccc(C)c5)c5cccc6c5oc5c(-c7ccccc7)cccc56)c5ccccc5c4oc3c3ccccc23)c2cccc3c2oc2c(-c4ccccc4)cccc23)c1 ; Cc1ccc(N(c2cc3c4cc(N(c5ccc(C)cc5)c5cccc6c5oc5c(-c7ccccc7)cccc56)c5ccccc5c4oc3c3ccccc23)c2cccc3c2oc2c(-c4ccccc4)cccc23)cc1]

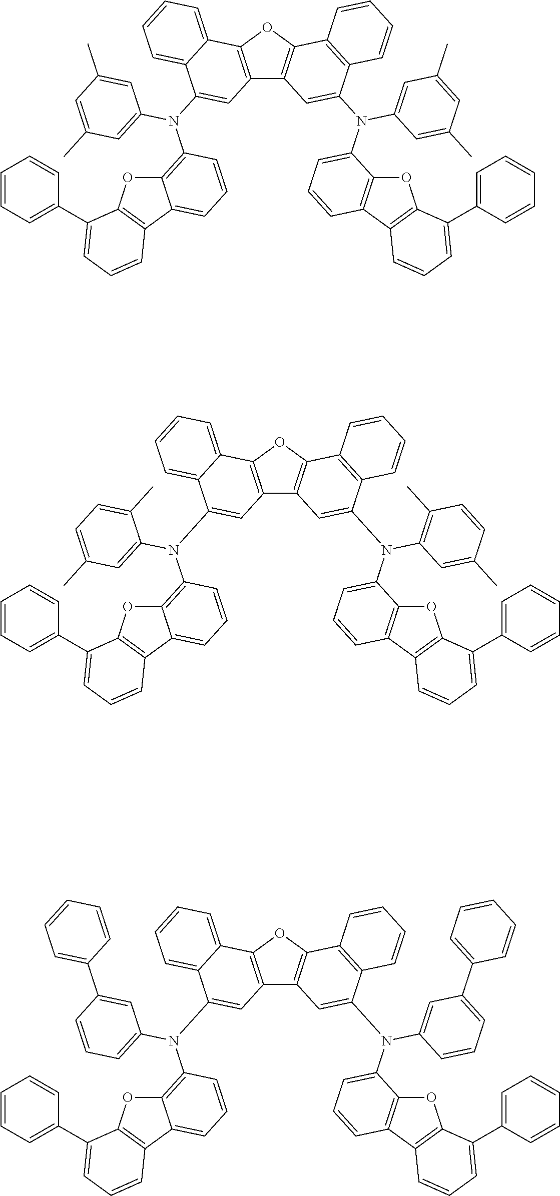 [SMILES: Cc1cc(C)cc(N(c2cc3c4cc(N(c5cc(C)cc(C)c5)c5cccc6c5oc5c(-c7ccccc7)cccc56)c5ccccc5c4oc3c3ccccc23)c2cccc3c2oc2c(-c4ccccc4)cccc23)c1 ; Cc1ccc(C)c(N(c2cc3c4cc(N(c5cc(C)ccc5C)c5cccc6c5oc5c(-c7ccccc7)cccc56)c5ccccc5c4oc3c3ccccc23)c2cccc3c2oc2c(-c4ccccc4)cccc23)c1 ; c1ccc(-c2cccc(N(c3cc4c5cc(N(c6cccc(-c7ccccc7)c6)c6cccc7c6oc6c(-c8ccccc8)cccc67)c6ccccc6c5oc4c4ccccc34)c3cccc4c3oc3c(-c5ccccc5)cccc34)c2)cc1]

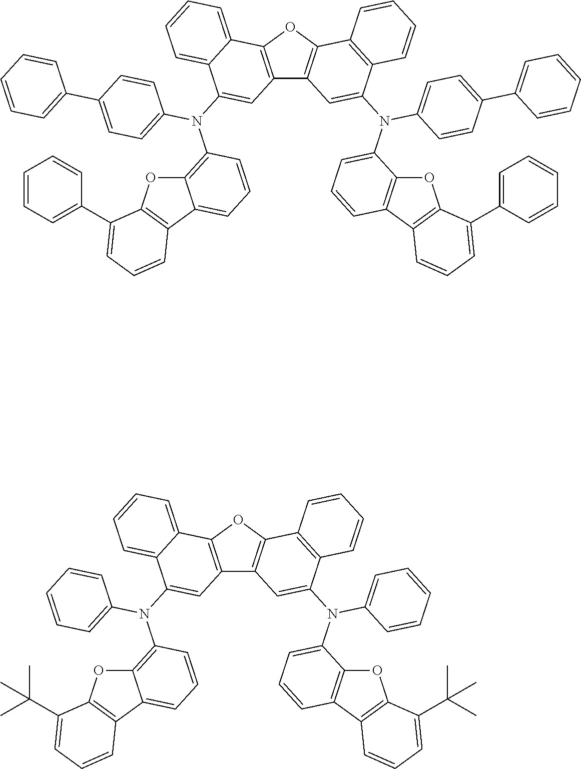 [SMILES: c1ccc(-c2ccc(N(c3cc4c5cc(N(c6ccc(-c7ccccc7)cc6)c6cccc7c6oc6c(-c8ccccc8)cccc67)c6ccccc6c5oc4c4ccccc34)c3cccc4c3oc3c(-c5ccccc5)cccc34)cc2)cc1 ; CC(C)(C)c1cccc2c1oc1c(N(c3ccccc3)c3cc4c5cc(N(c6ccccc6)c6cccc7c6oc6c(C(C)(C)C)cccc67)c6ccccc6c5oc4c4ccccc34)cccc12]

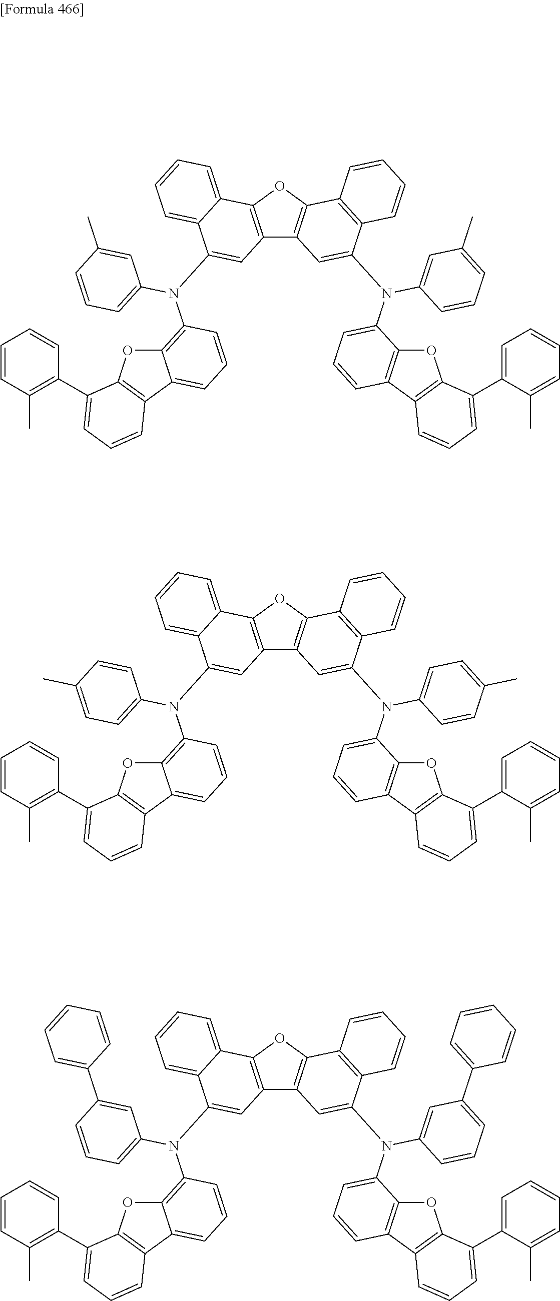 [SMILES: Cc1cccc(N(c2cc3c4cc(N(c5cccc(C)c5)c5cccc6c5oc5c(-c7ccccc7C)cccc56)c5ccccc5c4oc3c3ccccc23)c2cccc3c2oc2c(-c4ccccc4C)cccc23)c1 ; Cc1ccc(N(c2cc3c4cc(N(c5ccc(C)cc5)c5cccc6c5oc5c(-c7ccccc7C)cccc56)c5ccccc5c4oc3c3ccccc23)c2cccc3c2oc2c(-c4ccccc4C)cccc23)cc1 ; Cc1ccccc1-c1cccc2c1oc1c(N(c3cccc(-c4ccccc4)c3)c3cc4c5cc(N(c6cccc(-c7ccccc7)c6)c6cccc7c6oc6c(-c8ccccc8C)cccc67)c6ccccc6c5oc4c4ccccc34)cccc12]

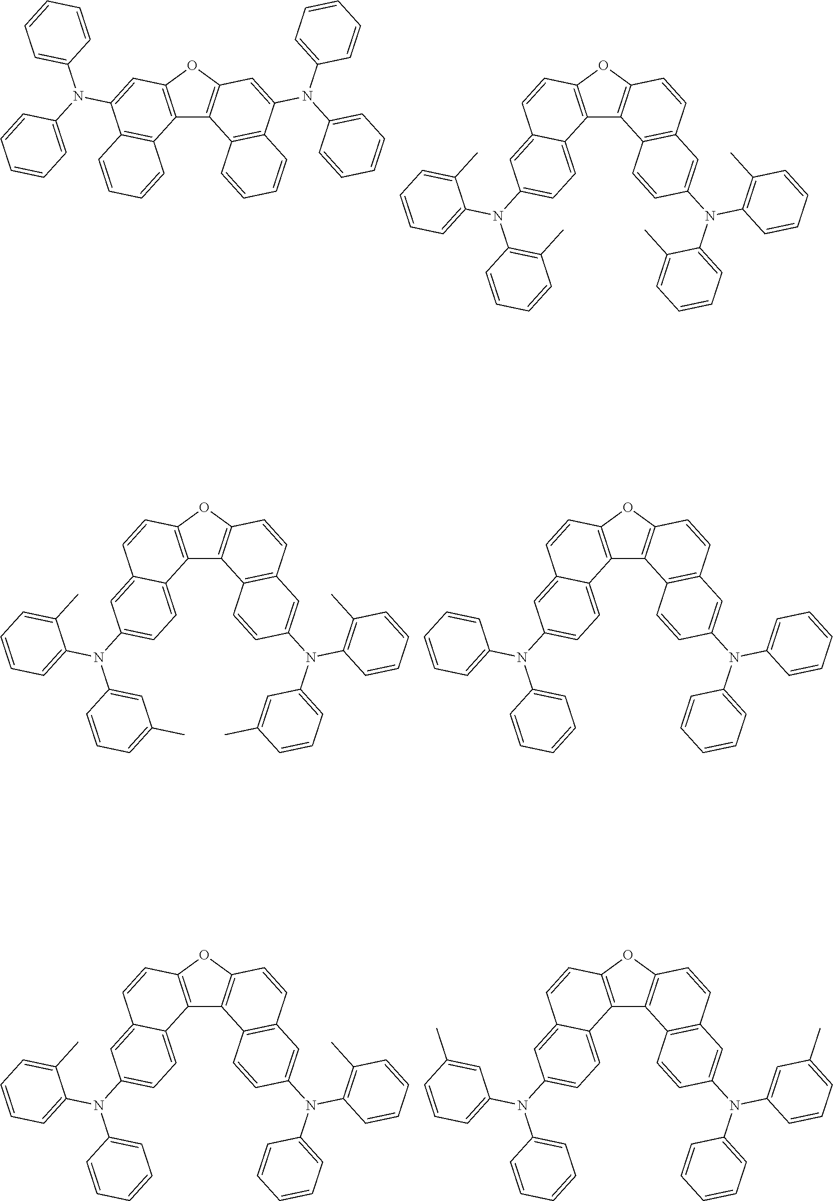

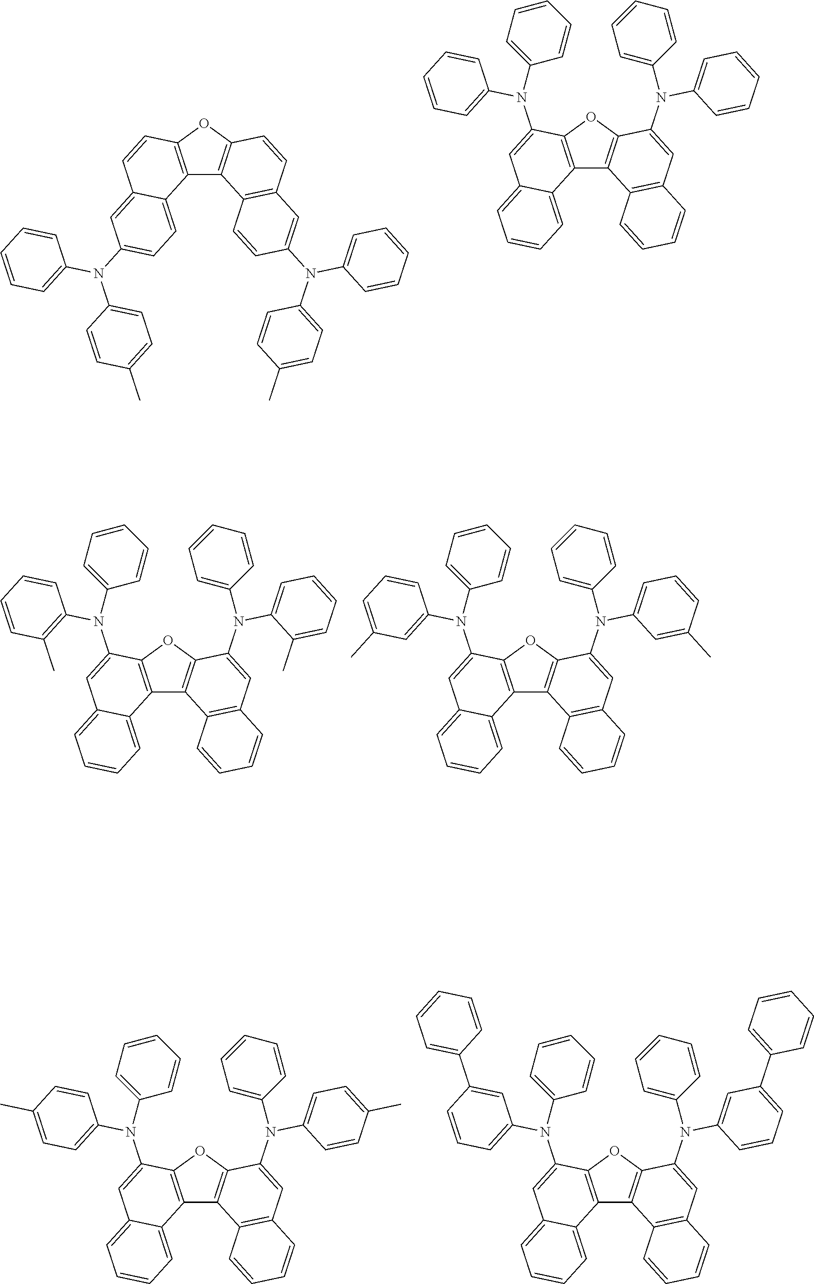

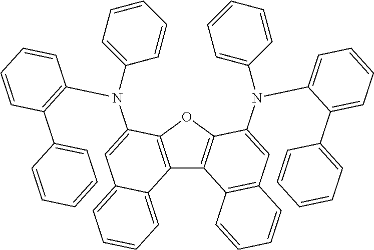 [SMILES: c1ccc(-c2ccccc2N(c2ccccc2)c2cc3ccccc3c3c2oc2c(N(c4ccccc4)c4ccccc4-c4ccccc4)cc4ccccc4c23)cc1]

### Compound Represented by Formula (9)

The compound represented by the formula (9) will be described below.

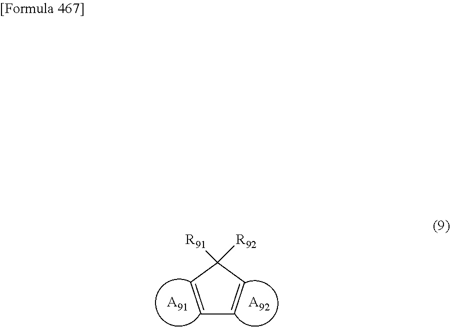

In the formula (9):
a ring Ar₉₁ and a ring Ar₉₂ are each independently a substituted or unsubstituted aromatic hydrocarbon ring having 6 to 50 ring carbon atoms, or a substituted or unsubstituted heterocycle having 5 to 50 ring atoms; and
at least one of the ring A₉₁ or the ring A₉₂ is bonded with * in a structure represented by a formula (92) below.

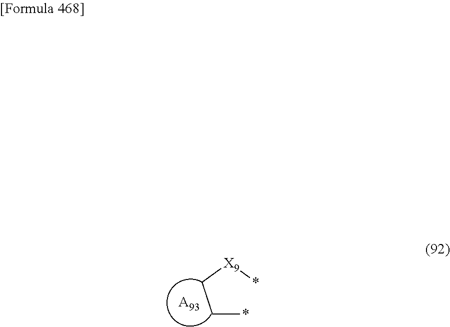

In the formula (92):
a ring A₉₃ is a substituted or unsubstituted aromatic hydrocarbon ring having 6 to 50 ring carbon atoms, or a substituted or unsubstituted heterocycle having 5 to 50 ring atoms;
X₉ is NR₉₃, C(R₉₄)(R₉₅), Si(R₉₆)(R₉₇), Ge(R₉₈)(R₉₉), an oxygen atom, a sulfur atom, or a selenium atom;
R₉₁ and R₉₂ are mutually bonded to form a substituted or unsubstituted monocyclic ring, mutually bonded to form a substituted or unsubstituted fused ring, or not mutually bonded; and
R₉₁ and R₉₂ forming neither the monocyclic ring nor the fused ring, and R₉₃ to R₉₉ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

At least one of the ring A₉₁ or the ring A₉₂ is bonded to * in a structure represented by the formula (92). In other words, the ring carbon atoms of the aromatic hydrocarbon ring or the ring atoms of the heterocycle of the ring A₉₁ in an exemplary embodiment are bonded to * in a structure represented by the formula (92). Further, the ring carbon atoms of the aromatic hydrocarbon ring or the ring atoms of the heterocycle of the ring A₉₂ in an exemplary embodiment are bonded to * in a structure represented by the formula (92).

In an exemplary embodiment, a group represented by a formula (93) below is bonded to one or both of the ring A₉₁ and the ring A₉₂.

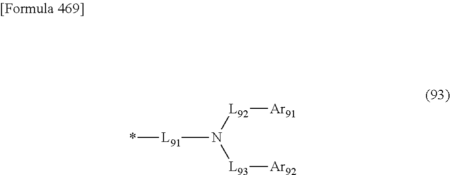

In the formula (93):
Ar₉₁ and Ar₉₂ are each independently a substituted or unsubstituted aryl group having 6 to 50 ring carbon atoms, or a substituted or unsubstituted heterocyclic group having 5 to 50 ring atoms;
L₉₁ to L₉₃ are each independently a single bond, a substituted or unsubstituted arylene group having 6 to 30 ring carbon atoms, a substituted or unsubstituted divalent heterocyclic group having 5 to 30 ring atoms, or a divalent linking group formed by bonding two, three or four groups selected from the group consisting of a substituted or unsubstituted arylene group having 6 to 30 ring carbon atoms and a substituted or unsubstituted divalent heterocyclic group having 5 to 30 ring atoms; and
* in the formula (93) represents a bonding position to one of the ring A₉₁ and the ring A₉₂.

In an exemplary embodiment, in addition to the ring A₉₁, the ring carbon atoms of the aromatic hydrocarbon ring or the ring atoms of the heterocycle of the ring A₉₂ are bonded to * in a structure represented by the formula (92). In this case, the structures represented by the formula (92) may be mutually the same or different.

In an exemplary embodiment, R₉₁ and R₉₂ are each independently a substituted or unsubstituted aryl group having 6 to 50 ring carbon atoms.

In an exemplary embodiment, R₉₁ and R₉₂ are mutually bonded to form a fluorene structure.

In an exemplary embodiment, the ring A₉₁ and the ring A₉₂ are each independently a substituted or unsubstituted aromatic hydrocarbon ring having 6 to 50 ring carbon atoms, an example of which is a substituted or unsubstituted benzene ring.

In an exemplary embodiment, the ring A₉₃ is a substituted or unsubstituted aromatic hydrocarbon ring having 6 to 50 ring carbon atoms, an example of which is a substituted or unsubstituted benzene ring.

In an exemplary embodiment, X₉ is an oxygen atom or a sulfur atom.

### Specific Examples of Compound Represented by Formula (9)

Specific examples of the compound represented by the formula (9) include compounds as below.

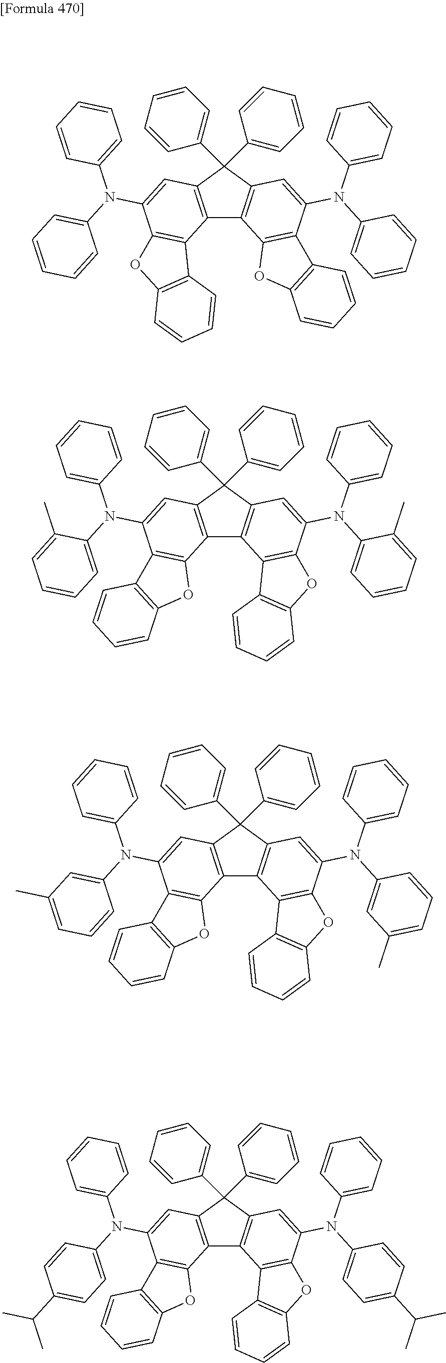 [SMILES: c1ccc(N(c2ccccc2)c2cc3c(c4c2oc2ccccc24)-c2c(cc(N(c4ccccc4)c4ccccc4)c4c2oc2ccccc24)C3(c2ccccc2)c2ccccc2)cc1 ; Cc1ccccc1N(c1ccccc1)c1cc2c(c3c1oc1ccccc13)-c1c(cc(N(c3ccccc3)c3ccccc3C)c3c1oc1ccccc13)C2(c1ccccc1)c1ccccc1 ; Cc1cccc(N(c2ccccc2)c2cc3c(c4c2oc2ccccc24)-c2c(cc(N(c4ccccc4)c4cccc(C)c4)c4c2oc2ccccc24)C3(c2ccccc2)c2ccccc2)c1 ; CC(C)c1ccc(N(c2ccccc2)c2cc3c(c4c2oc2ccccc24)-c2c(cc(N(c4ccccc4)c4ccc(C(C)C)cc4)c4c2oc2ccccc24)C3(c2ccccc2)c2ccccc2)cc1]

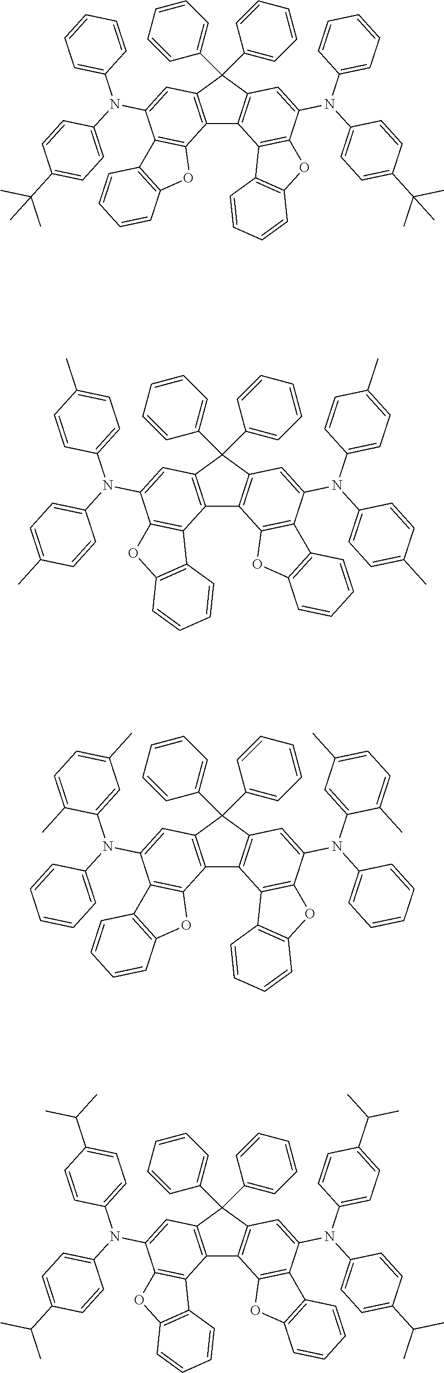

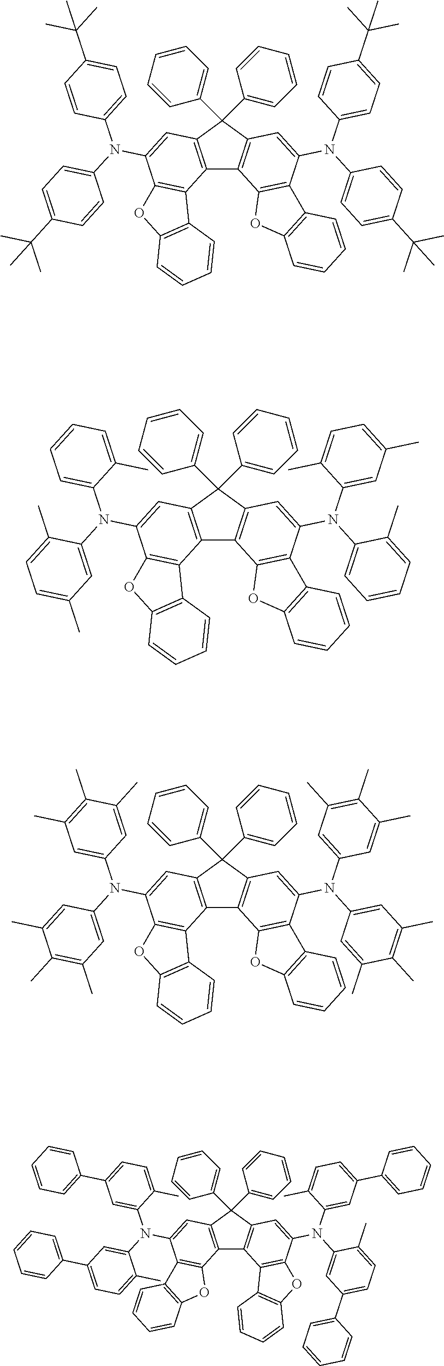

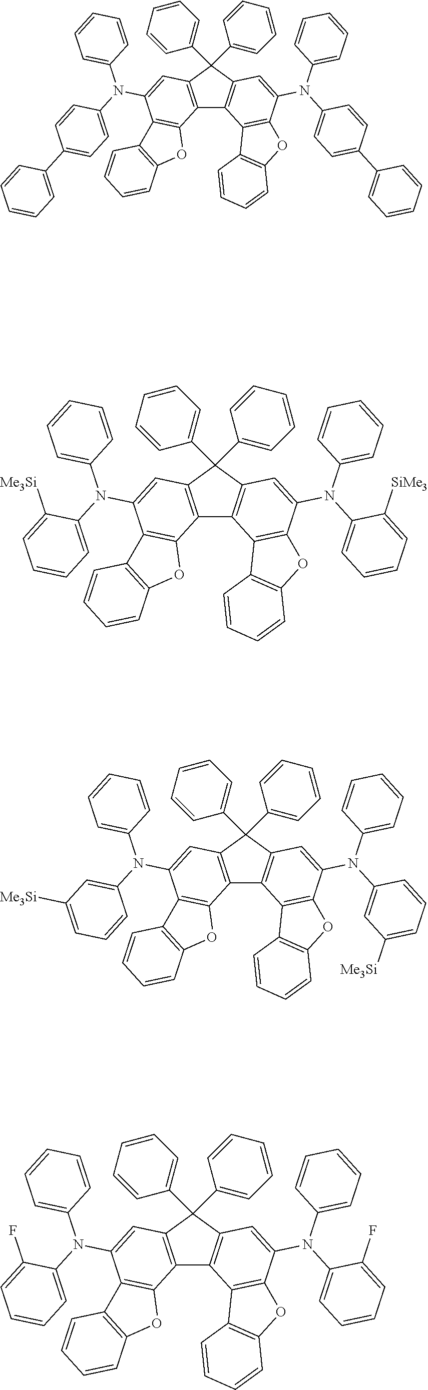 [SMILES: c1ccc(-c2ccc(N(c3ccccc3)c3cc4c(c5c3oc3ccccc35)-c3c(cc(N(c5ccccc5)c5ccc(-c6ccccc6)cc5)c5c3oc3ccccc35)C4(c3ccccc3)c3ccccc3)cc2)cc1 ; C[Si](C)(C)c1ccccc1N(c1ccccc1)c1cc2c(c3c1oc1ccccc13)-c1c(cc(N(c3ccccc3)c3ccccc3[Si](C)(C)C)c3c1oc1ccccc13)C2(c1ccccc1)c1ccccc1 ; C[Si](C)(C)c1cccc(N(c2ccccc2)c2cc3c(c4c2oc2ccccc24)-c2c(cc(N(c4ccccc4)c4cccc([Si](C)(C)C)c4)c4c2oc2ccccc24)C3(c2ccccc2)c2ccccc2)c1 ; Fc1ccccc1N(c1ccccc1)c1cc2c(c3c1oc1ccccc13)-c1c(cc(N(c3ccccc3)c3ccccc3F)c3c1oc1ccccc13)C2(c1ccccc1)c1ccccc1]

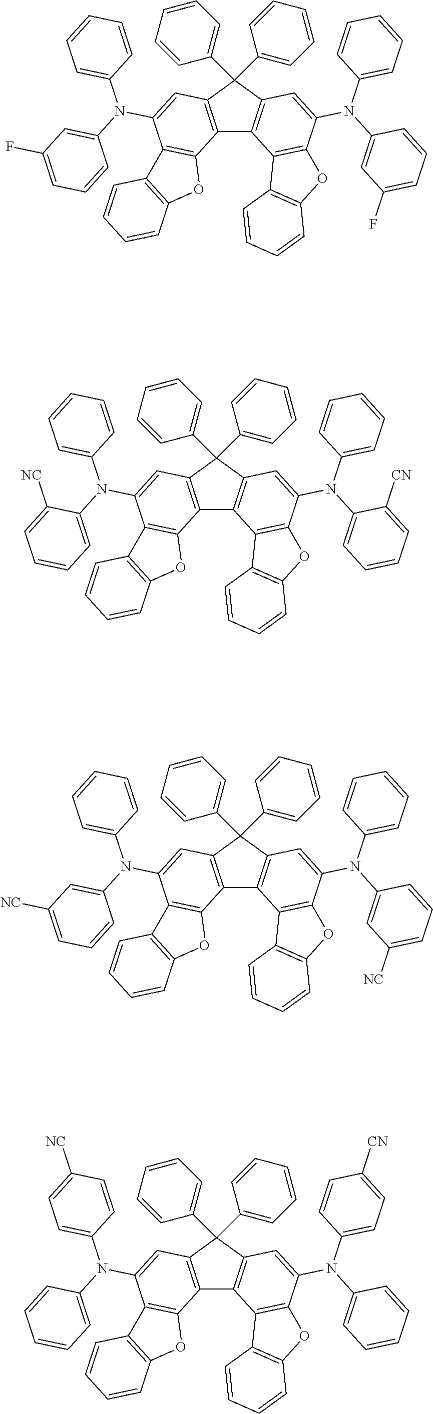 [SMILES: Fc1cccc(N(c2ccccc2)c2cc3c(c4c2oc2ccccc24)-c2c(cc(N(c4ccccc4)c4cccc(F)c4)c4c2oc2ccccc24)C3(c2ccccc2)c2ccccc2)c1 ; N#Cc1ccccc1N(c1ccccc1)c1cc2c(c3c1oc1ccccc13)-c1c(cc(N(c3ccccc3)c3ccccc3C#N)c3c1oc1ccccc13)C2(c1ccccc1)c1ccccc1 ; N#Cc1cccc(N(c2ccccc2)c2cc3c(c4c2oc2ccccc24)-c2c(cc(N(c4ccccc4)c4cccc(C#N)c4)c4c2oc2ccccc24)C3(c2ccccc2)c2ccccc2)c1 ; N#Cc1ccc(N(c2ccccc2)c2cc3c(c4c2oc2ccccc24)-c2c(cc(N(c4ccccc4)c4ccc(C#N)cc4)c4c2oc2ccccc24)C3(c2ccccc2)c2ccccc2)cc1]

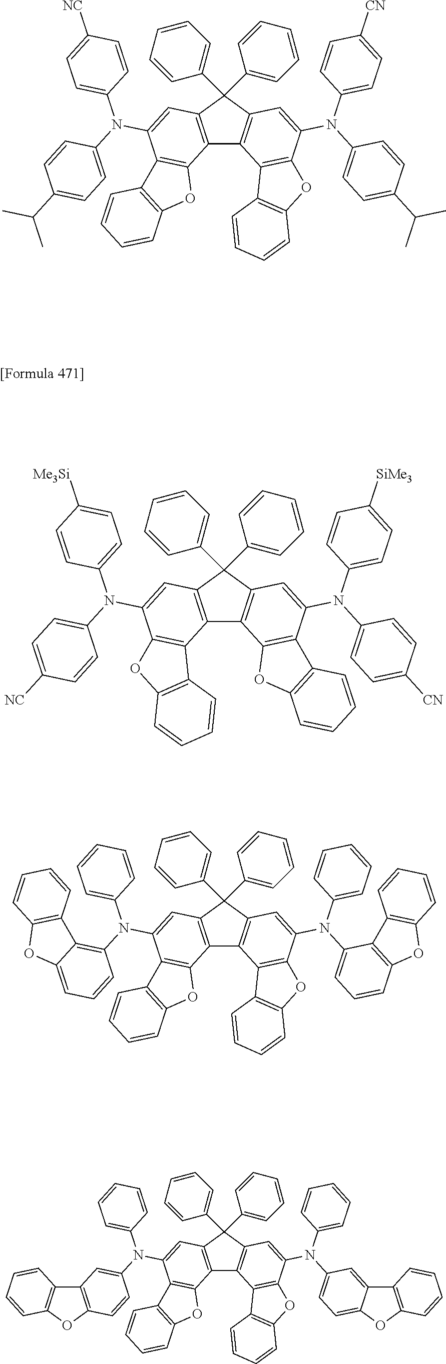

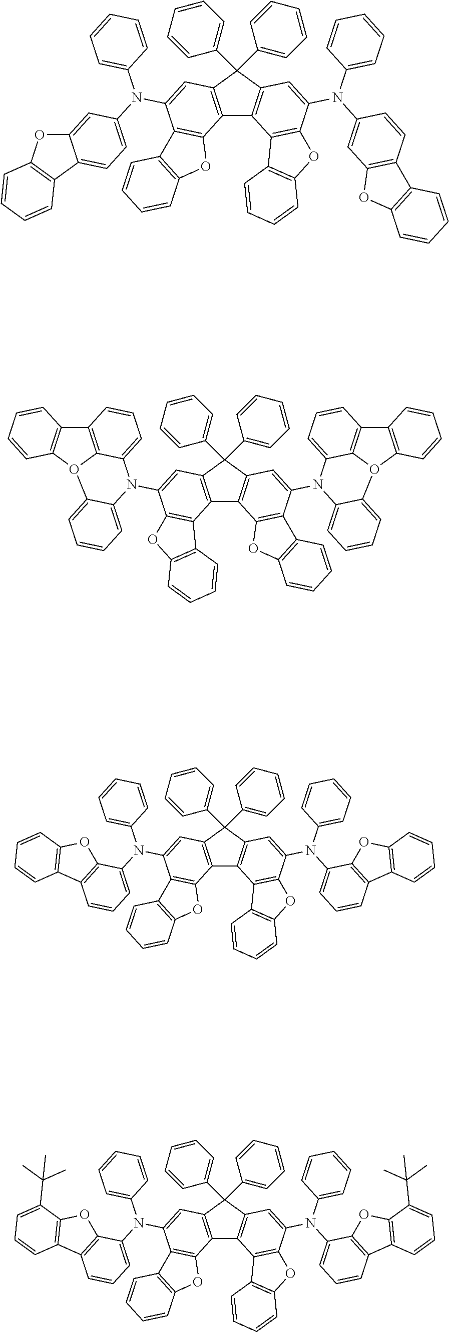

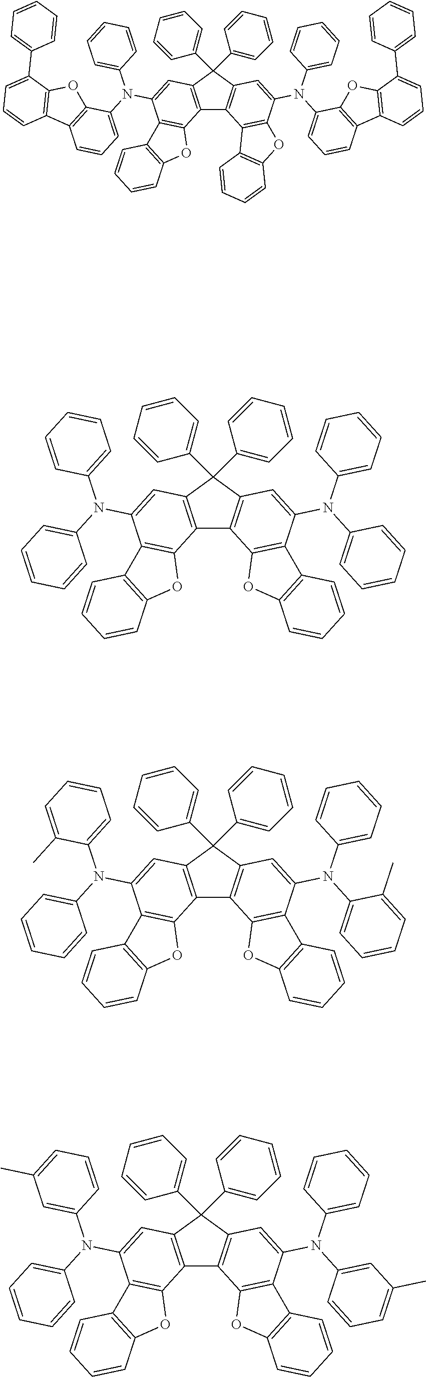

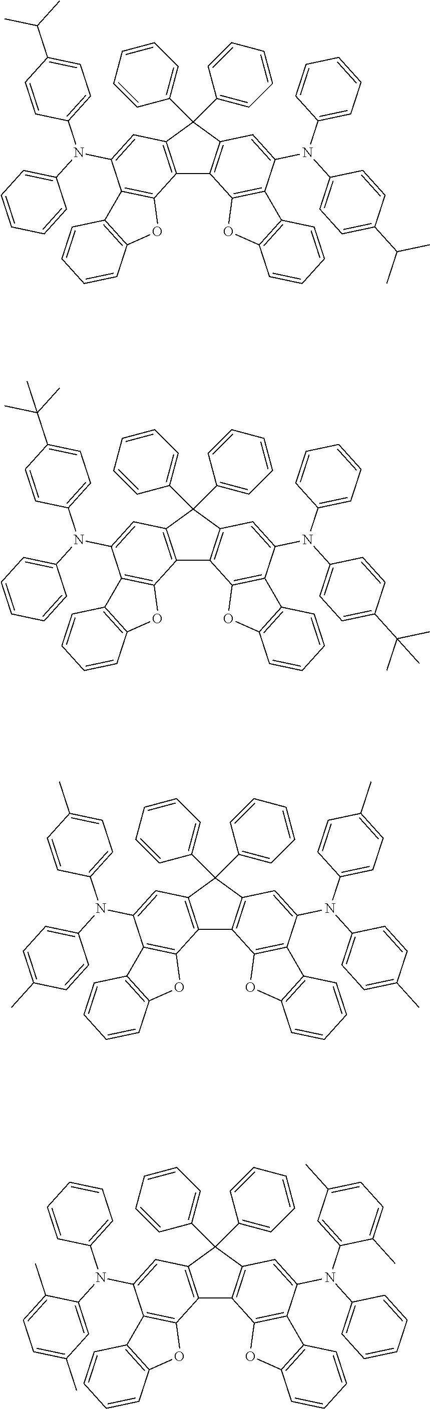

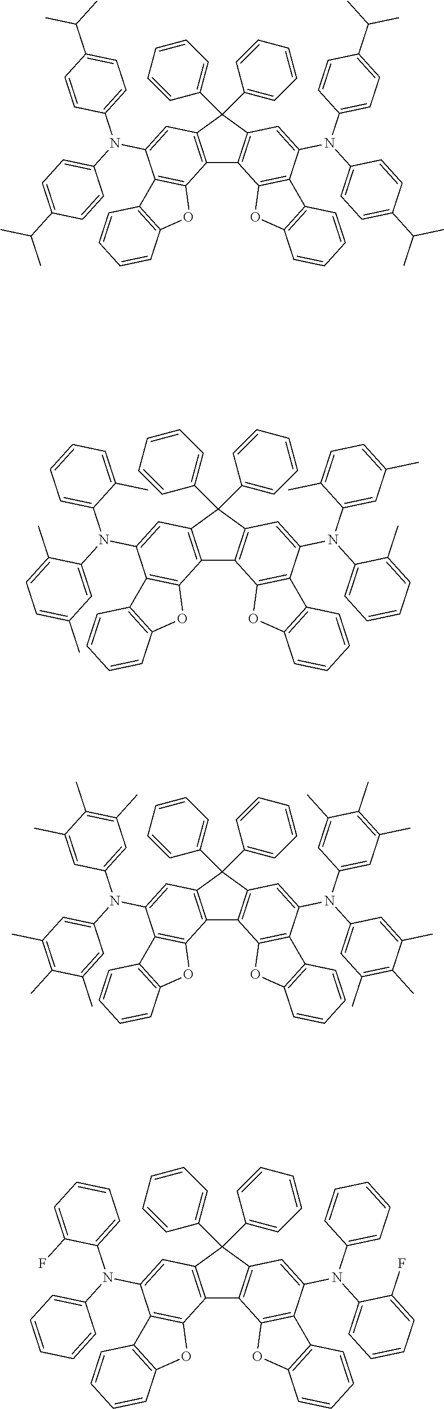

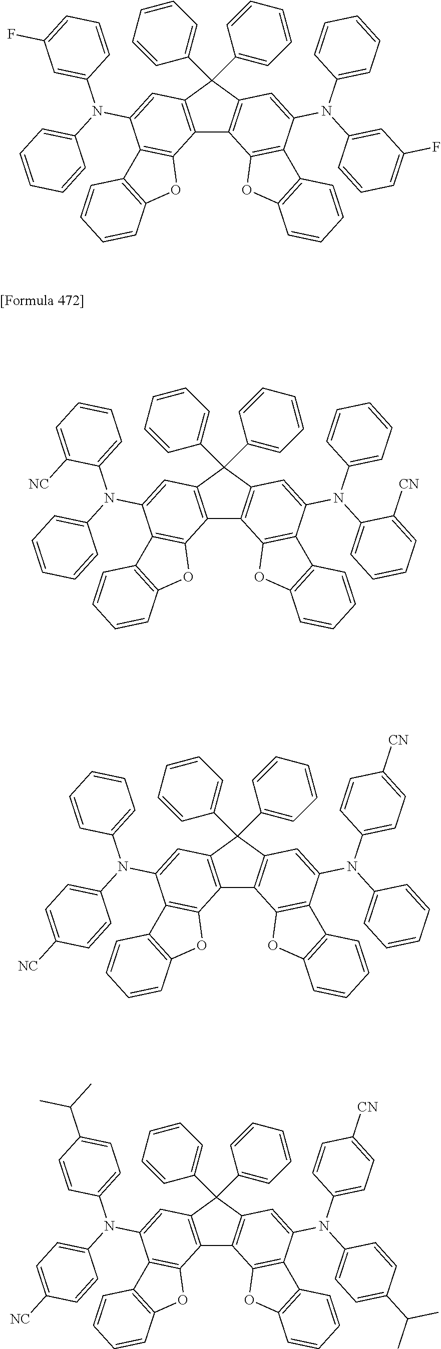 [SMILES: Fc1cccc(N(c2ccccc2)c2cc3c(c4oc5ccccc5c24)-c2c(cc(N(c4ccccc4)c4cccc(F)c4)c4c2oc2ccccc24)C3(c2ccccc2)c2ccccc2)c1 ; N#Cc1ccccc1N(c1ccccc1)c1cc2c(c3oc4ccccc4c13)-c1c(cc(N(c3ccccc3)c3ccccc3C#N)c3c1oc1ccccc13)C2(c1ccccc1)c1ccccc1 ; N#Cc1ccc(N(c2ccccc2)c2cc3c(c4oc5ccccc5c24)-c2c(cc(N(c4ccccc4)c4ccc(C#N)cc4)c4c2oc2ccccc24)C3(c2ccccc2)c2ccccc2)cc1 ; CC(C)c1ccc(N(c2ccc(C#N)cc2)c2cc3c(c4oc5ccccc5c24)-c2c(cc(N(c4ccc(C#N)cc4)c4ccc(C(C)C)cc4)c4c2oc2ccccc24)C3(c2ccccc2)c2ccccc2)cc1]

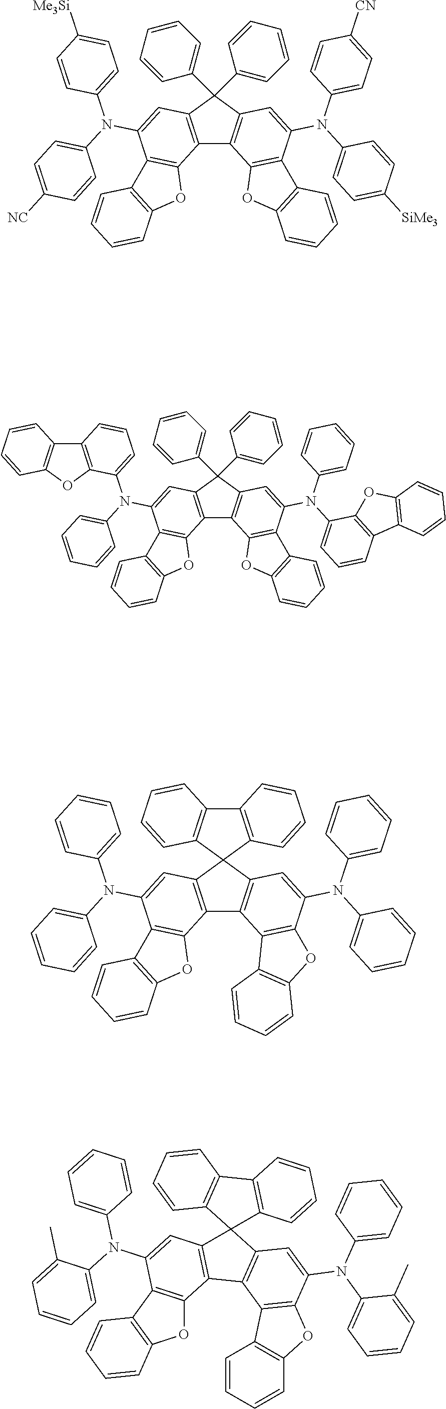 [SMILES: C[Si](C)(C)c1ccc(N(c2ccc(C#N)cc2)c2cc3c(c4oc5ccccc5c24)-c2c(cc(N(c4ccc(C#N)cc4)c4ccc([Si](C)(C)C)cc4)c4c2oc2ccccc24)C3(c2ccccc2)c2ccccc2)cc1 ; c1ccc(N(c2cccc3c2oc2ccccc23)c2cc3c(c4oc5ccccc5c24)-c2c(cc(N(c4ccccc4)c4cccc5c4oc4ccccc45)c4c2oc2ccccc24)C3(c2ccccc2)c2ccccc2)cc1 ; c1ccc(N(c2ccccc2)c2cc3c(c4c2oc2ccccc24)-c2c(cc(N(c4ccccc4)c4ccccc4)c4c2oc2ccccc24)C32c3ccccc3-c3ccccc32)cc1 ; Cc1ccccc1N(c1ccccc1)c1cc2c(c3c1oc1ccccc13)-c1c(cc(N(c3ccccc3)c3ccccc3C)c3c1oc1ccccc13)C21c2ccccc2-c2ccccc21]

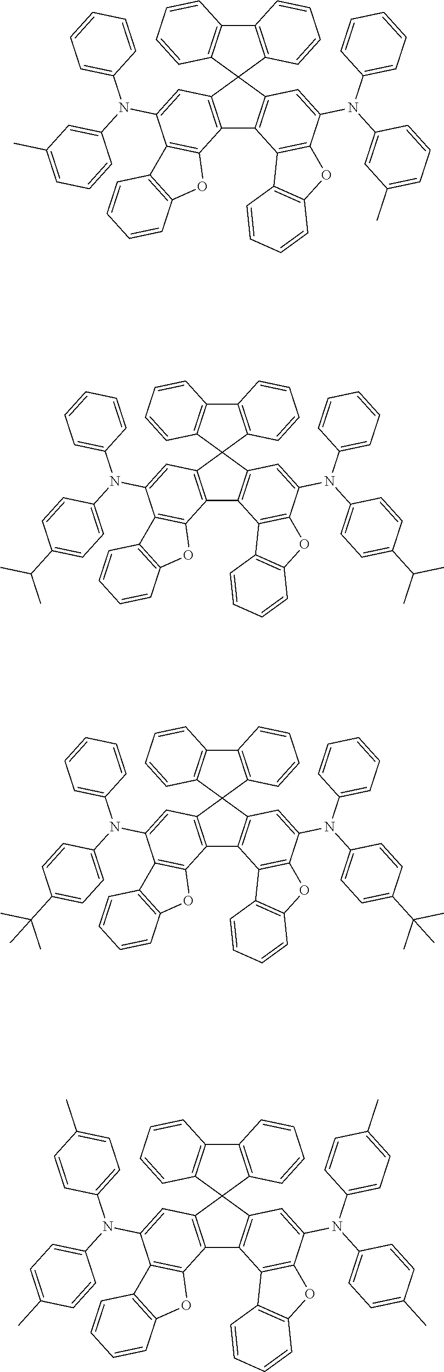

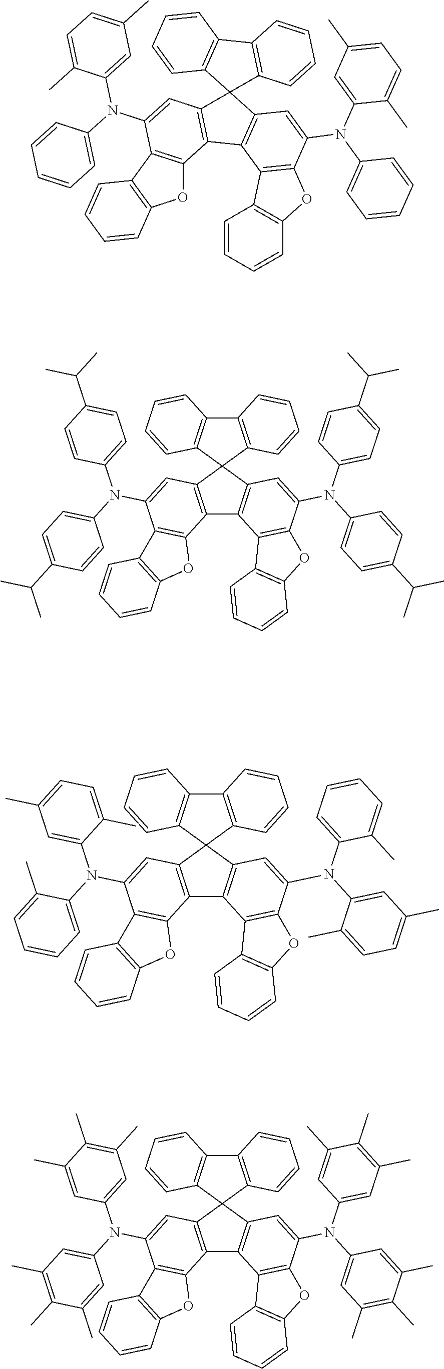

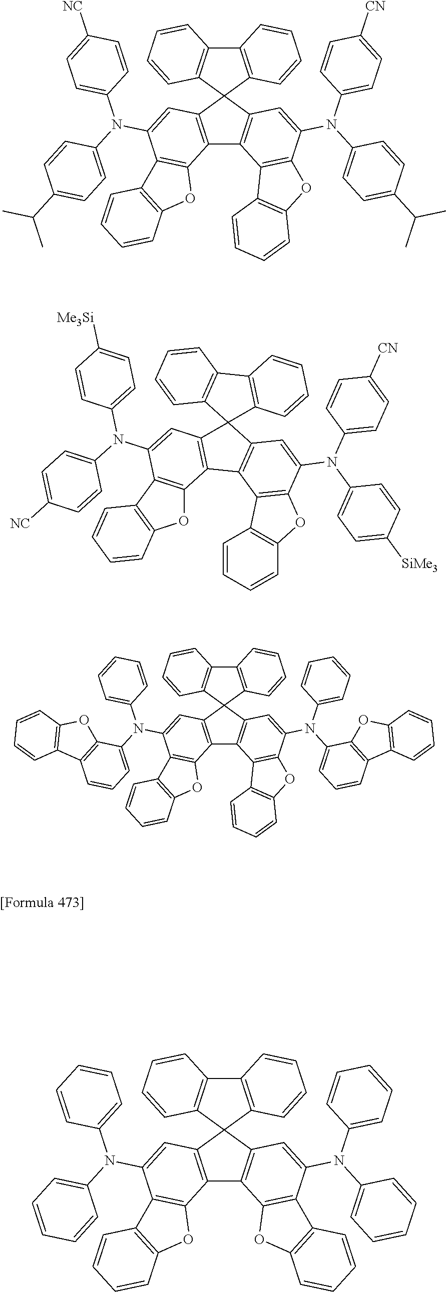

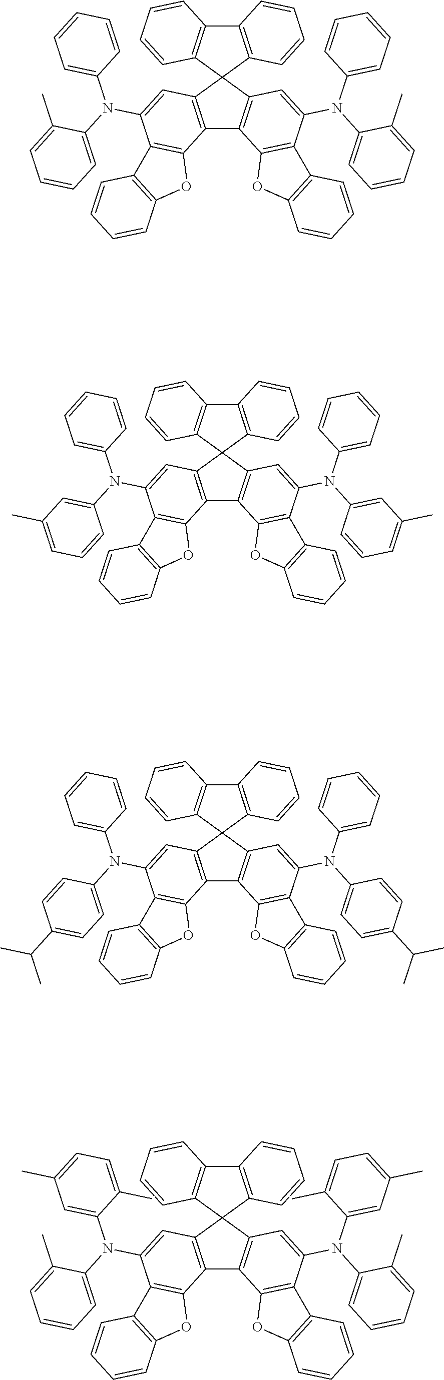

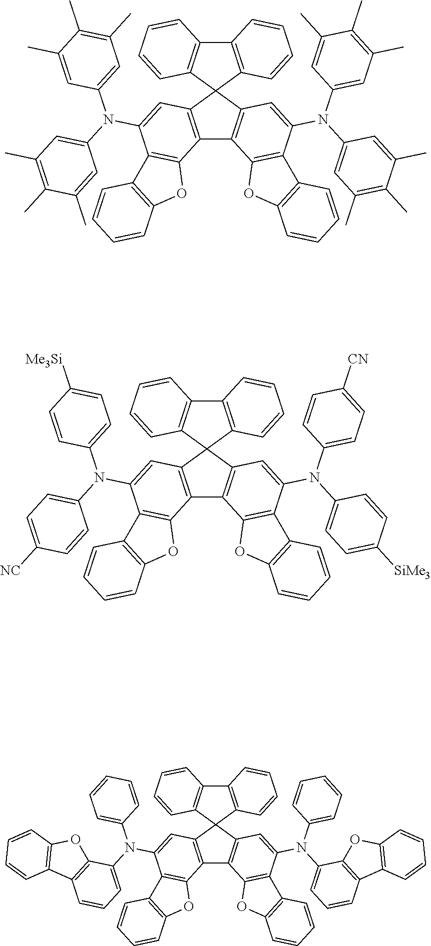

### Compound Represented by Formula (10)

The compound represented by the formula (10) will be described below.

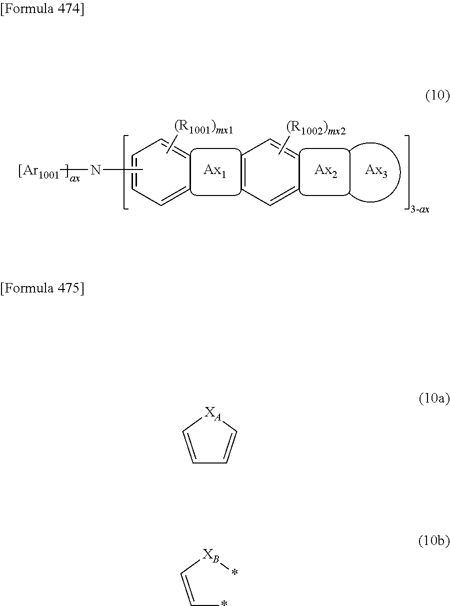

In the formula (10):
a ring Ax₁ is a ring represented by the formula (10a) that is fused with adjacent ring(s) at any position(s);
a ring Ax₂ is a ring represented by the formula (10b) that is fused with adjacent ring(s) at any position(s);
two * in the formula (10b) are bonded to a ring Ax₃ at any positions;
X_{A} and X_{B} are each independently C(R₁₀₀₃)(R₁₀₀₄), Si(R₁₀₀₅)(R₁₀₀₆), an oxygen atom or a sulfur atom;
the ring AX₃ is a substituted or unsubstituted aromatic hydrocarbon ring having 6 to 50 ring carbon atoms, or a substituted or unsubstituted heterocycle having 5 to 50 ring atoms;
A₁₀₀₁ is a substituted or unsubstituted aryl group having 6 to 50 ring carbon atoms, or a substituted or unsubstituted heterocyclic group having 5 to 50 ring atoms;
R₁₀₀₁ to R₁₀₀₆ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms;
mx1 is 3, and mx2 is 2;
a plurality of R₁₀₀₁ are mutually the same or different;
a plurality of R₁₀₀₂ are mutually the same or different;
ax is 0, 1, or 2;
when ax is 0 or 1, the structures enclosed by brackets indicated by “3-ax” are mutually the same or different; and
when ax is 2, a plurality of Ar₁₀₀₁ are mutually the same or different.

In an exemplary embodiment, Ar₁₀₀₁ is a substituted or unsubstituted aryl group having 6 to 50 ring carbon atoms.

In an exemplary embodiment, the ring Ax₃ is a substituted or unsubstituted aromatic hydrocarbon ring having 6 to 50 ring carbon atoms, an example of which is a substituted or unsubstituted benzene ring, a substituted or unsubstituted naphthalene ring, or a substituted or unsubstituted anthracene ring.

In an exemplary embodiment, R₁₀₀₃ and R₁₀₀₄ are each independently a substituted or unsubstituted alkyl group having 1 to 50 carbon atoms.

In an exemplary embodiment, ax is 1.

### Specific Examples of Compound Represented by Formula (10)

Specific examples of the compound represented by the formula (10) include compounds as below.

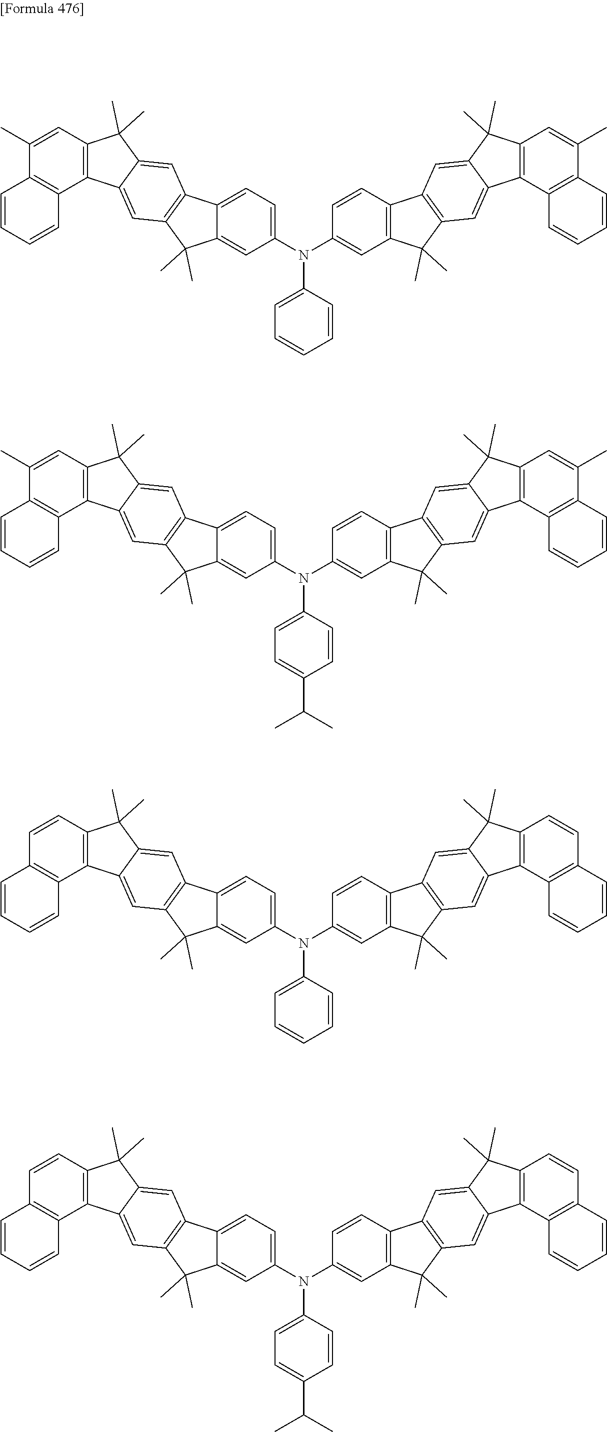

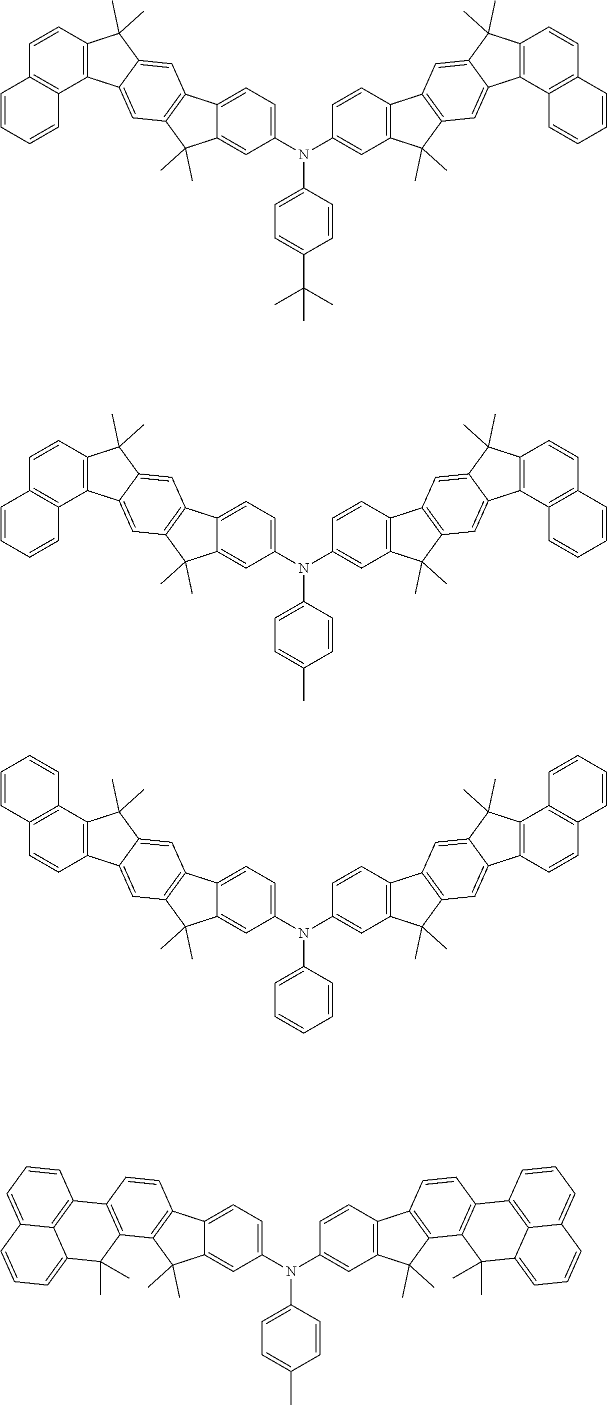 [SMILES: CC(C)(C)c1ccc(N(c2ccc3c(c2)C(C)(C)c2cc4c(cc2-3)C(C)(C)c2ccc3ccccc3c2-4)c2ccc3c(c2)C(C)(C)c2cc4c(cc2-3)C(C)(C)c2ccc3ccccc3c2-4)cc1 ; Cc1ccc(N(c2ccc3c(c2)C(C)(C)c2cc4c(cc2-3)C(C)(C)c2ccc3ccccc3c2-4)c2ccc3c(c2)C(C)(C)c2cc4c(cc2-3)C(C)(C)c2ccc3ccccc3c2-4)cc1 ; CC1(C)c2cc(N(c3ccccc3)c3ccc4c(c3)C(C)(C)c3cc5c(cc3-4)C(C)(C)c3c-5ccc4ccccc34)ccc2-c2cc3c(cc21)-c1ccc2ccccc2c1C3(C)C ; Cc1ccc(N(c2ccc3c(c2)C(C)(C)c2c-3ccc3c2C(C)(C)c2cccc4cccc-3c24)c2ccc3c(c2)C(C)(C)c2c-3ccc3c2C(C)(C)c2cccc4cccc-3c24)cc1]

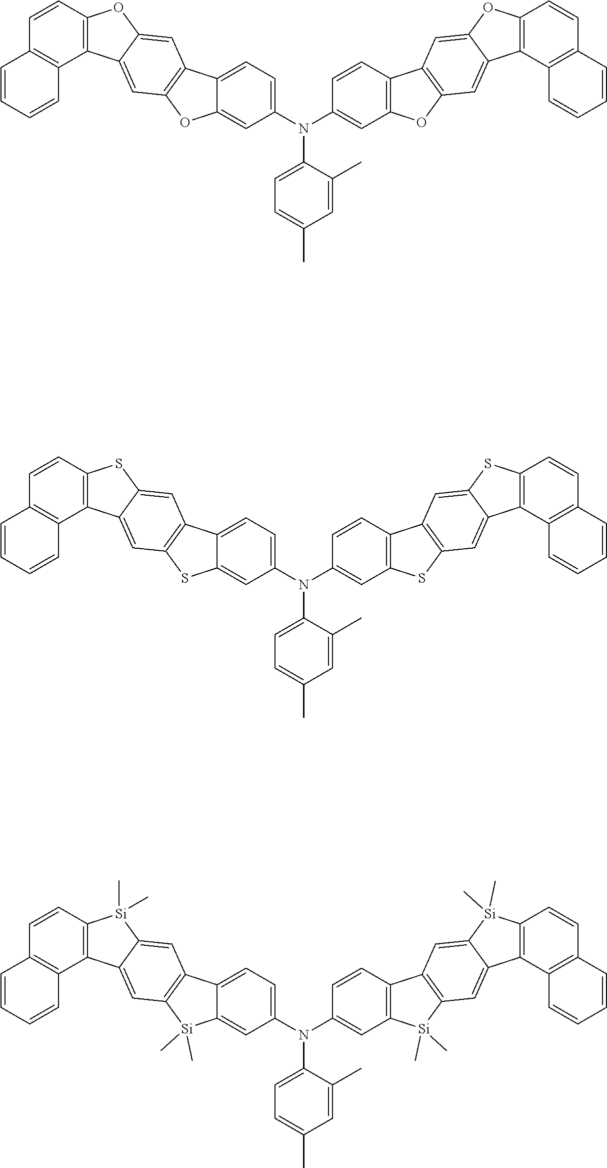 [SMILES: Cc1ccc(N(c2ccc3c(c2)oc2cc4c(cc23)oc2ccc3ccccc3c24)c2ccc3c(c2)oc2cc4c(cc23)oc2ccc3ccccc3c24)c(C)c1 ; Cc1ccc(N(c2ccc3c(c2)sc2cc4c(cc23)sc2ccc3ccccc3c24)c2ccc3c(c2)sc2cc4c(cc23)sc2ccc3ccccc3c24)c(C)c1 ; Cc1ccc(N(c2ccc3c(c2)[Si](C)(C)c2cc4c(cc2-3)[Si](C)(C)c2ccc3ccccc3c2-4)c2ccc3c(c2)[Si](C)(C)c2cc4c(cc2-3)[Si](C)(C)c2ccc3ccccc3c2-4)c(C)c1]

In an exemplary embodiment, the substituent for “the substituted or unsubstituted” group in each of the formulae is an unsubstituted alkyl group having 1 to 50 carbon atoms, an unsubstituted aryl group having 6 to 50 ring carbon atoms, or an unsubstituted heterocyclic group having 5 to 50 ring atoms.

In an exemplary embodiment, the substituent for “the substituted or unsubstituted” group in each of the formulae is an unsubstituted alkyl group having 1 to 18 carbon atoms, an unsubstituted aryl group having 6 to 18 ring carbon atoms, or an unsubstituted heterocyclic group having 5 to 18 ring atoms.

### Electron Transporting Zone

An exemplary arrangement of the organic EL device according to the exemplary embodiment further includes an electron transporting zone disposed between the cathode and the emitting region. The electron transporting zone includes at least one electron transporting layer. The at least one electron transporting layer in the electron transporting zone contains a nitrogen-containing compound having at least one of a five-membered ring containing a nitrogen atom or a six-membered ring containing a nitrogen atom.

In an exemplary arrangement of the organic EL device of the exemplary embodiment, at least one electron transporting layer in the electron transporting zone contains, as a nitrogen-containing compound, at least one compound selected from the group consisting of an imidazole derivative, a benzimidazole derivative, an azine derivative, a carbazole derivative and a phenanthroline derivative.

### Phenanthroline Derivative

In an exemplary arrangement of the organic EL device of the exemplary embodiment, at least one electron transporting layer in the electron transporting zone contains, as a nitrogen-containing compound, a phenanthroline derivative.

In an exemplary arrangement of the organic EL device of the exemplary embodiment, the phenanthroline derivative (phenanthroline compound) contained in the electron transporting layer is a compound that has at least one group represented by a formula (21) below and is represented by a formula (20) below.

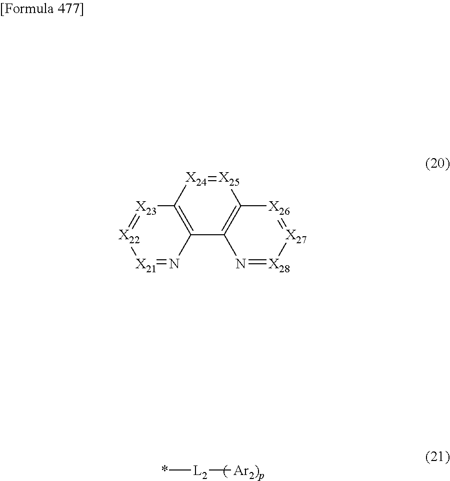

In the formula (20):
X₂₁ to X₂₈ are each independently a nitrogen atom, CR₂₁, or a carbon atom bonded to a group represented by the formula (21);
at least one of X₂₁ to X₂₈ is a carbon atom bonded to a group represented by the formula (21);
when a plurality of groups represented by the formula (21) are present, the plurality of groups represented by the formula (21) are mutually the same or different;
at least one combination of adjacent two or more of a plurality of R₂₁ are mutually bonded to form a substituted or unsubstituted monocyclic ring, mutually bonded to form a substituted or unsubstituted fused ring, or not mutually bonded; and
R₂₁ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₉₃₁, a group represented by —COOR₉₃₂, a group represented by —S(═O)₂R₉₃₃, a group represented by —B(R₉₃₄)(R₉₃₅), a group represented by —P(═O)(R₉₃₆)(R₉₃₇), a halogen atom, a cyano group, a nitro group, a substituted or unsubstituted aryl group having 6 to 50 ring carbon atoms, or a substituted or unsubstituted heterocyclic group having 5 to 50 ring atoms.

In the formula (21):
Ar₂ is a substituted or unsubstituted aryl group having 6 to 50 ring carbon atoms, or a substituted or unsubstituted heterocyclic group having 5 to 50 ring atoms;
p is 1, 2, 3, 4, or 5;
when two or more Ar₂ are present, the two or more Ar₂ are mutually the same or different;
L₂ is a single bond or a linking group;
L₂ as the linking group is a substituted or unsubstituted polyvalent linear, branched or cyclic aliphatic hydrocarbon group having 1 to 50 carbon atoms, a substituted or unsubstituted polyvalent aromatic hydrocarbon group having 6 to 50 ring carbon atoms, a substituted or unsubstituted polyvalent heterocyclic group having 5 to 50 ring atoms, or a polyvalent multiple linking group provided by bonding two or three groups selected from the polyvalent aromatic hydrocarbon ring group and the polyvalent heterocyclic group;
the aromatic hydrocarbon ring group and the heterocyclic group forming L₂ as the multiple linking group are mutually the same or different, and adjacent ones thereof are mutually bonded to form a ring, or not mutually bonded;
Ar₂ and L₂ as the linking group are mutually bonded to form a ring, or not mutually bonded;
L₂ as the linking group, and a carbon atom in one of X₂₁ to X₂₈ adjacent to a carbon atom bonded to L₂, or R₂₁ in CR₂₁ are mutually bonded to form a ring, or not mutually bonded; and
* in the formula (21) represents a bonding position to a ring represented by the formula (20);
in the phenanthroline compound, R₉₀₁, R₉₀₂, R₉₀₃, R₉₀₄, R₉₀₅, R₉₀₆, R₉₀₇, R₉₃₁, R₉₃₂, R₉₃₃, R₉₃₄, R₉₃₅, R₉₃₆ and R₉₃₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different;
when a plurality of R₉₀₇ are present, the plurality of R₉₀₇ are mutually the same or different;
when a plurality of R₉₃₁ are present, the plurality of R₉₃₁ are mutually the same or different;
when a plurality of R₉₃₂ are present, the plurality of R₉₃₂ are mutually the same or different;
when a plurality of R₉₃₃ are present, the plurality of R₉₃₃ are mutually the same or different;
when a plurality of R₉₃₄ are present, the plurality of R₉₃₄ are mutually the same or different;
when a plurality of R₉₃₅ are present, the plurality of R₉₃₅ are mutually the same or different;
when a plurality of R₉₃₆ are present, the plurality of R₉₃₆ are mutually the same or different; and
when a plurality of R₉₃₇ are present, the plurality of R₉₃₇ are mutually the same or different.

A group represented by —O—(R₉₀₄) herein in which R₉₀₄ is a hydrogen atom is a hydroxy group.

A group represented by —S—(R₉₀₅) herein in which R₉₀₅ is a hydrogen atom is a thiol group.

A group represented by —S(═O)₂R₉₃₃ herein in which R₉₃₃ is a substituent is a substituted sulfo group.

A group represented by —B(R₉₃₄)(R₉₃₅) herein in which R₉₃₄ and R₉₃₅ are each a substituent is a substituted boryl group.

A group represented by —P(═O)(R₉₃₆)(R₉₃₇) herein in which R₉₃₆ and R₉₃₇ are each a substituent is a substituted phosphine oxide group, and a group represented by —P(═O)(R₉₃₆)(R₉₃₇) herein in which R₉₃₆ and R₉₃₇ are each an aryl group is an aryl phosphoryl group.

An “unsubstituted polyvalent linear, branched or cyclic aliphatic hydrocarbon group” mentioned herein has, unless otherwise specified herein, 1 to 50, preferably 1 to 20, more preferably 1 to 6 carbon atoms.

An “unsubstituted polyvalent aromatic hydrocarbon group” mentioned herein has, unless otherwise specified herein, 6 to 50, preferably 6 to 30, more preferably 6 to 18 ring carbon atoms.

An “unsubstituted polyvalent heterocyclic group” mentioned herein has, unless otherwise specified herein, 5 to 50, preferably 5 to 30, more preferably 5 to 18 ring atoms.

In an exemplary embodiment, a heterocyclic group having 5 to 50 ring atoms in Ar₂ of the formula (21) contains a substituted or unsubstituted group derived from a cyclic structure represented by the formula (20).

In an exemplary embodiment, X₂₁ and X₂₈ in the formula (20) are each a carbon atom bonded to a group represented by the formula (21).

In an exemplary embodiment, one of X₂₁ and X₂₈ in the formula (20) is a carbon atom bonded to a group represented by the formula (21), and the other of X₂₁ and X₂₈ in the formula (20) is a carbon atom bonded to a hydrogen atom.

In an exemplary embodiment, X₂₁ to X₂₈ in the formula (20) are each independently CR₂₁ or a carbon atom bonded to a group represented by the formula (21).

In an exemplary embodiment, X₂₁ to X₂₈ in the formula (20) not being the carbon atom bonded to the group represented by the formula (21) are each CR₂₁. In other words, in an exemplary embodiment, the compound represented by the formula (20) is a 1,10-phenanthroline derivative.

In an exemplary embodiment, Ar₂ in the formula (21) is a substituted or unsubstituted fused aromatic hydrocarbon group having 8 to 20 ring carbon atoms.

In an exemplary embodiment, the fused aromatic hydrocarbon group having 8 to 20 ring carbon atoms is, for instance, a group derived from any one of aromatic hydrocarbons selected from the group consisting of naphthalene, anthracene, acephenanthrylene, aceanthrylene, benzoanthracene, triphenylene, pyrene, chrysene, naphthacene, fluorene, phenanthrene, fluoranthene and benzofluoranthene.

In an exemplary embodiment, Ar₂ in the formula (21) is a substituted or unsubstituted anthryl group.

In an exemplary embodiment, Ar₂ in the formula (21) is a substituted or unsubstituted heterocyclic group having 5 to 40 ring carbon atoms.

In an exemplary embodiment, Ar₂ in the formula (21) is a substituted or unsubstituted group derived from the cyclic structure represented by the formula (20).

In an exemplary embodiment, Ar₂ in the formula (21) is a group represented by a formula (23) below.

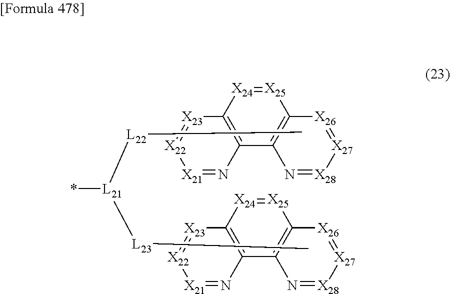

In the formula (23):
X₂₁ to X₂₈ are each independently a nitrogen atom, CR₂₁, a group represented by the formula (21), or a carbon atom bonded to L₂₂ or L₂₃;
L₂₁ is a linking group, and L₂₁ as the linking group is a substituted or unsubstituted trivalent linear, branched or cyclic aliphatic hydrocarbon group having 1 to 50 carbon atoms, a substituted or unsubstituted trivalent aromatic hydrocarbon group having 6 to 50 ring carbon atoms, or a substituted or unsubstituted trivalent heterocyclic group having 5 to 50 ring atoms; and
L₂₂ and L₂₃ are each independently a single bond or a linking group, and L₂₂ and L₂₃ as the linking group are each independently a substituted or unsubstituted divalent linear, branched or cyclic aliphatic hydrocarbon group having 1 to 50 carbon atoms, a substituted or unsubstituted arylene group having 6 to 50 ring carbon atoms, or a substituted or unsubstituted divalent heterocyclic group having 5 to 50 ring atoms.

In the formula of the phenanthroline compound:
when a plurality of X₂₁ are present, the plurality of X₂₁ are mutually the same or different;
when a plurality of X₂₂ are present, the plurality of X₂₂ are mutually the same or different;
when a plurality of X₂₃ are present, the plurality of X₂₃ are mutually the same or different;
when a plurality of X₂₄ are present, the plurality of X₂₄ are mutually the same or different;
when a plurality of X₂₅ are present, the plurality of X₂₅ are mutually the same or different;
when a plurality of X₂₆ are present, the plurality of X₂₆ are mutually the same or different;
when a plurality of X₂₇ are present, the plurality of X₂₇ are mutually the same or different; and
when a plurality of X₂₈ are present, the plurality of X₂₈ are mutually the same or different.

In an exemplary embodiment, X₂₁ to X₂₈ in the formula (23) are preferably each independently a nitrogen atom, CR₂₁, or a carbon atom bonded to L₂₂ or L₂₃, more preferably CR₂₁ or a carbon atom bonded to L₂₂ or L₂₃.

In an exemplary embodiment, the phenanthroline compound is a compound represented by a formula (24) below.

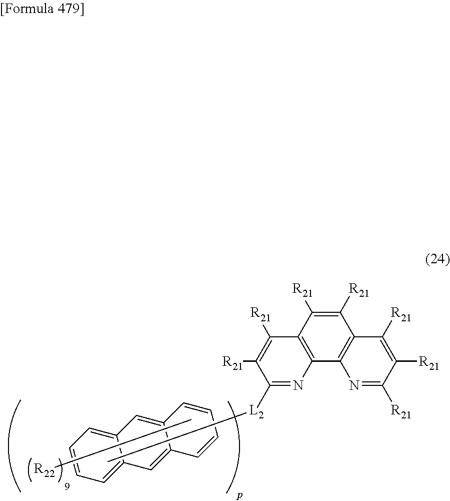

In the formula (24):
a plurality of R₂₁ each independently represent the same as R₂₁ in the formula (20);
a plurality of R₂₂ each independently represent the same as R₂₁ in the formula (20);
L₂ represents the same as L₂ in the formula (21);
p is 1, 2, 3, 4, or 5; and
a plurality of R₂₂ and L₂ are each bonded to a carbon atom at one of positions 1 to 10 of an anthracene ring.

In an exemplary embodiment, the phenanthroline compound is a compound represented by a formula (24A) below.

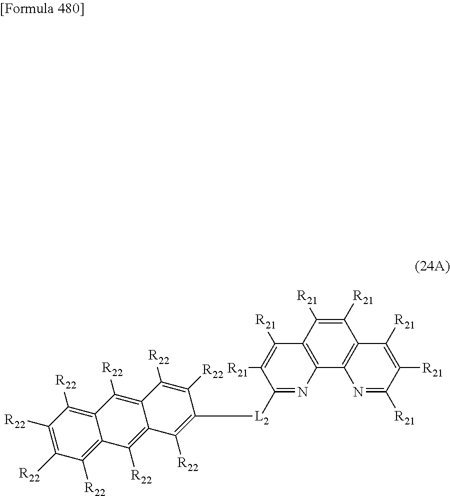

In the formula (24A):
a plurality of R₂₁ each independently represent the same as R₂₁ in the formula (20);
a plurality of R₂₂ each independently represent the same as R₂₁ in the formula (20); and
L₂ represents the same as L₂ in the formula (21).

In an exemplary embodiment, the phenanthroline compound is a compound represented by a formula (24B) below.

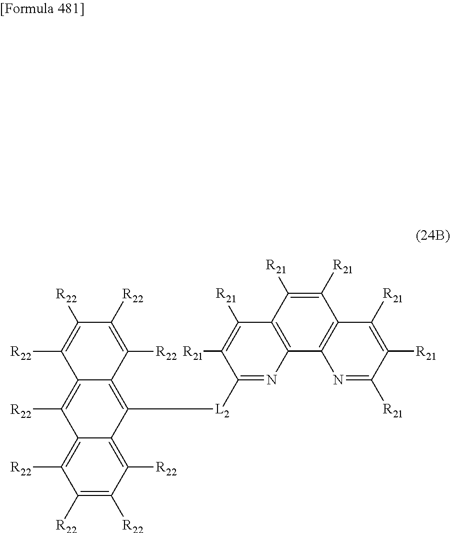

In the formula (24B):
a plurality of R₂₁ each independently represent the same as R₂₁ in the formula (20);
a plurality of R₂₂ each independently represent the same as R₂₁ in the formula (20); and
L₂ represents the same as L₂ in the formula (21).

In an exemplary embodiment, the phenanthroline compound is a compound represented by a formula (25) below.

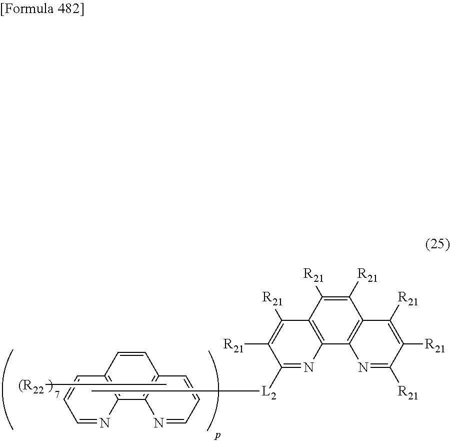

In the formula (25):
a plurality of R₂₁ each independently represent the same as R₂₁ in the formula (20);
a plurality of R₂₂ each independently represent the same as R₂₁ in the formula (20);
L₂ represents the same as L₂ in the formula (21);
p is 1, 2, 3, 4, or 5; and
a plurality of R₂₂ and L₂ are each bonded to a carbon atom at one of positions 2 to 9 of a phenanthroline ring.

In an exemplary embodiment, the phenanthroline compound is a compound represented by a formula (25A) below.

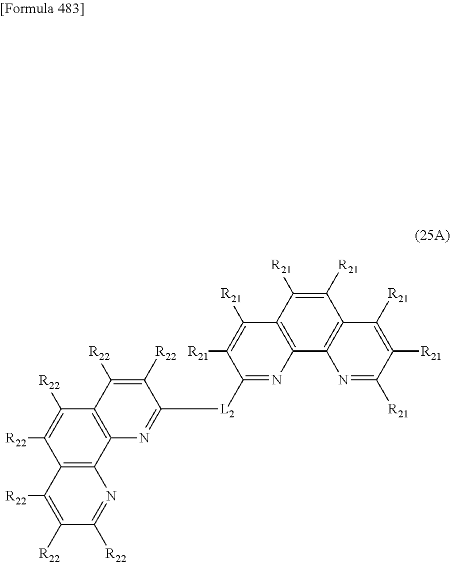

In the formula (25A):
a plurality of R₂₁ each independently represent the same as R₂₁ in the formula (20);
a plurality of R₂₂ each independently represent the same as R₂₁ in the formula (20); and
L₂ represents the same as L₂ in the formula (21).

In an exemplary embodiment, L₂ in the formulae (24), (24A), (24B), (25) and (25A) is a single bond, a substituted or unsubstituted arylene group having 6 to 50 ring carbon atoms, or a substituted or unsubstituted divalent heterocyclic group having 5 to 50 ring atoms.

In an exemplary embodiment, the phenanthroline compound is a compound represented by a formula (25B) below.

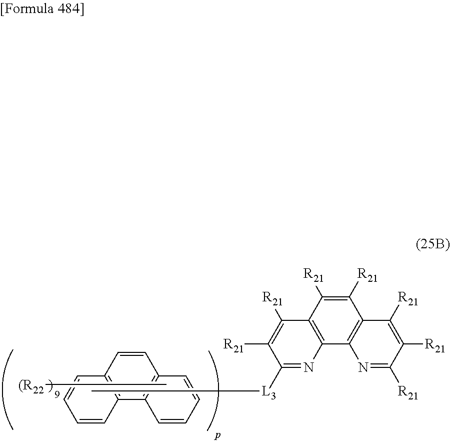

In the formula (25B):
a plurality of R₂₁ each independently represent the same as R₂₁ in the formula (20);
a plurality of R₂₂ each independently represent the same as R₂₁ in the formula (20);
L₃ is a linking group, and L₃ as the linking group is a substituted or unsubstituted polyvalent linear, branched or cyclic aliphatic hydrocarbon group having 1 to 50 carbon atoms, a substituted or unsubstituted polyvalent amino group, a substituted or unsubstituted polyvalent aromatic hydrocarbon ring group having 6 to 50 ring carbon atoms, a substituted or unsubstituted polyvalent heterocyclic group having 5 to 50 ring atoms, or a polyvalent multiple linking group provided by bonding two or three groups selected from the polyvalent aromatic hydrocarbon ring group and the polyvalent heterocyclic group;
the aromatic hydrocarbon ring group and the heterocyclic group forming L₃ as the multiple linking group are mutually the same or different, and adjacent ones thereof are mutually bonded to form a ring, or not mutually bonded;
p is 1, 2, 3, 4, or 5; and
a plurality of R₂₂ and L₃ are each bonded to a carbon atom at one of positions 1 to 10 of a phenanthrene ring.

In an exemplary embodiment, the phenanthroline compound is a compound represented by a formula (25C) below.

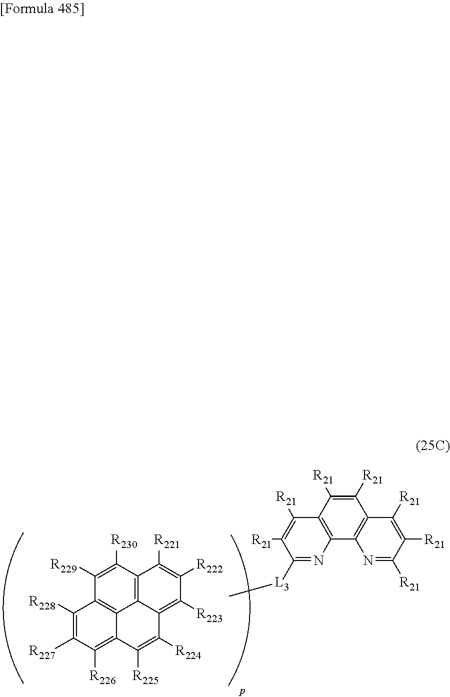

In the formula (25C):
a plurality of R₂₁ each independently represent the same as R₂₁ in the formula (20);
one of R₂₂₁ to R₂₃₀ is a single bond with L₃, and R₂₂₁ to R₂₃₀ not being the single bond with L₃ each independently represent the same as R₂₁ in the formula (20);
L₃ is a linking group, and L₃ as the linking group represents the same as L₃ as the linking group in the formula (25B); and
p is 1, 2, 3, 4, or 5.

In an exemplary embodiment, the phenanthroline compound is a compound represented by a formula (25D) below.

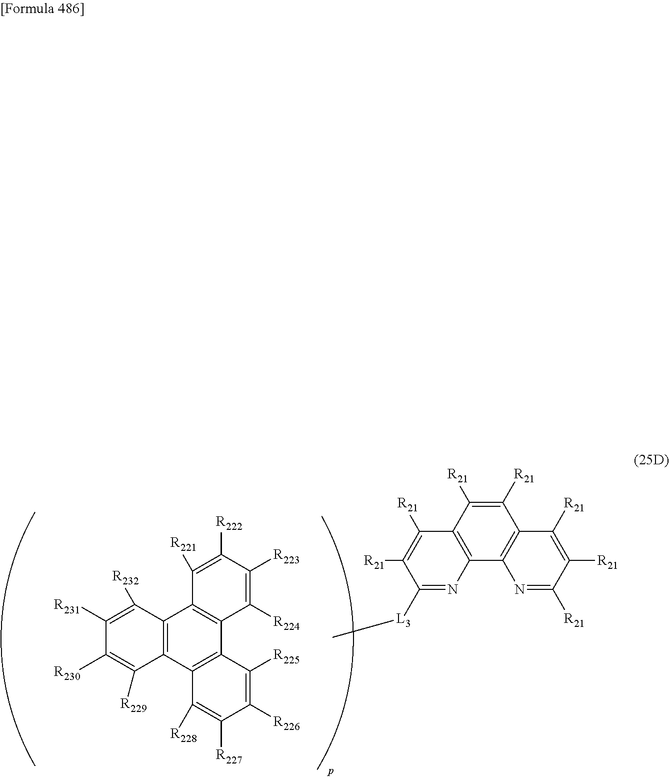

In the formula (25D):
a plurality of R₂₁ each independently represent the same as R₂₁ in the formula (20);
one of R₂₂₁ to R₂₃₂ is a single bond with L₃, and R₂₂₁ to R₂₃₂ not being the single bond with L₃ each independently represent the same as R₂₁ in the formula (20);
L₃ is a linking group, and L₃ as the linking group represents the same as L₃ as the linking group in the formula (25B); and
p is 1, 2, 3, 4, or 5.

In an exemplary embodiment, the phenanthroline compound is a compound represented by a formula (25E) below.

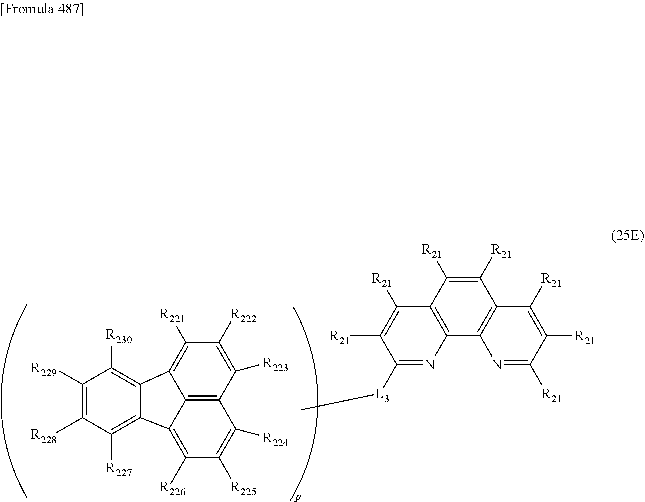

In the formula (25E):
a plurality of R₂₁ each independently represent the same as R₂₁ in the formula (20);
one of R₂₂₁ to R₂₃₀ is a single bond with L₃, and R₂₂₁ to R₂₃₀ not being the single bond with L₃ each independently represent the same as R₂₁ in the formula (20);
L₃ is a linking group, and L₃ as the linking group represents the same as L₃ as the linking group in the formula (25B); and
p is 1, 2, 3, 4, or 5.

Also preferably, L₃ in the formulae (25B), (25C), (25D) and (25E) are each a substituted or unsubstituted arylene group having 6 to 50 ring carbon atoms, or a substituted or unsubstituted divalent heterocyclic group having 5 to 50 ring atoms.

### Method for Producing Phenanthroline Compound

The phenanthroline compound can be produced by a known method. The phenanthroline compound also can be produced based on a known method through a known alternative reaction using a known material(s) tailored for the target compound.

### Azine Derivative

In an exemplary arrangement of the organic EL device according to the exemplary embodiment, at least one electron transporting layer in the electron transporting zone contains, as a nitrogen-containing compound, an azine derivative.

In an exemplary arrangement of the organic EL device according to the exemplary embodiment, the azine derivative contained in the electron transporting layer is a compound represented by a formula (E42) below.

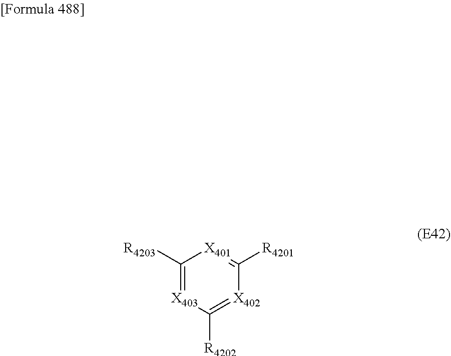

In the formula (E42):
X₄₀₁ to X₄₀₃ are each independently CR₄₂₀₄ or a nitrogen atom;
at least one of X₄₀₁ to X₄₀₃ is a nitrogen atom;
when two or more R₄₂₀₄ are present, the two or more R₄₂₀₄ are mutually the same or different; and
R₄₂₀₁ to R₄₂₀₄ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

R₉₀₁ to R₉₀₇ in the azine derivative are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different; and
when a plurality of R₉₀₇ are present, the plurality of R₉₀₇ are mutually the same or different.

In an exemplary arrangement of the organic EL device according to the exemplary embodiment, the azine derivative contained in the electron transporting layer is a compound represented by a formula (E421) below.

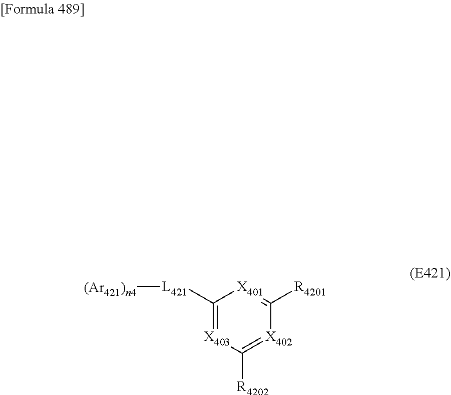

In the formula (E421):
X₄₀₁ to X₄₀₃ and R₄₂₀₁ and R₄₂₀₂ each independently represent the same as X₄₀₁ to X₄₀₃ and R₄₂₀₁ and R₄₂₀₂ in the formula (E42);
n4 is 1, 2, or 3;
L₄₂₁ is a single bond, a substituted or unsubstituted arylene group having 6 to 50 ring carbon atoms, a trivalent group or tetravalent group derived from the arylene group, a substituted or unsubstituted divalent heterocyclic group having 5 to 50 ring atoms, a trivalent group or tetravalent group derived from the divalent heterocyclic group, or a divalent group formed by bonding two groups selected from the group consisting of a substituted or unsubstituted arylene group having 6 to 50 ring carbon atoms, and a substituted or unsubstituted divalent heterocyclic group having 5 to 50 ring atoms, or a trivalent group or tetravalent group derived from the divalent group;
A₄₂₁ is a substituted or unsubstituted aryl group having 6 to 50 ring carbon atoms, or a substituted or unsubstituted heterocyclic group having 5 to 50 ring atoms;
when n4 is 2 or 3, Ar₄₂₁ are mutually the same or different; and
when n4 is 2 or 3, L₄₂₁ is not a single bond.

In an exemplary arrangement of the organic EL device according to the exemplary embodiment, the azine derivative contained in the electron transporting layer is a compound represented by a formula (E422) and a compound represented by a formula (E423) below.

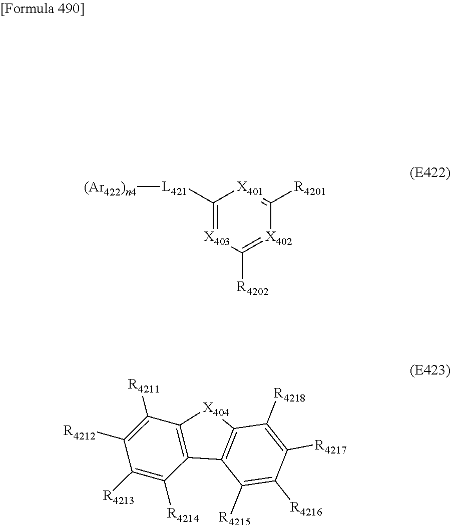

In the formula (E422):
n4, X₄₀₁ to X₄₀₃, R₄₂₀₁ and R₄₂₀₂ each independently represent the same as n4, X₄₀₁ to X₄₀₃, R₄₂₀₁ and R₄₂₀₂ in the formula (E42);
L₄₂₁ represents the same as L₄₂₁ in the formula (E421); and
Ar₄₂₂ is a group represented by the formula (E423).

In the formula (E423):
X₄₀₄ is an oxygen atom, a sulfur atom, N(R₄₂₂₁) or C(R₄₂₂₂)(R₄₂₂₃);
one of R₄₂₁₁ to R₄₂₁₈ and R₄₂₂₁ to R₄₂₂₃ is a single bond with L₄₂₁;
at least one combination of adjacent two or more of R₄₂₁₁ to R₄₂₁₈ not being the single bond with L₄₂₁ are mutually bonded to form a substituted or unsubstituted monocyclic ring, mutually bonded to form a substituted or unsubstituted fused ring, or not mutually bonded;
a combination of R₄₂₂₂ and R₄₂₂₃ not being the single bond with L₄₂₁ are mutually bonded to form a substituted or unsubstituted monocyclic ring, mutually bonded to form a substituted or unsubstituted fused ring, or not mutually bonded;
R₄₂₁₁ to R₄₂₁₈ not being the single bond with L₄₂₁ and forming neither the substituted or unsubstituted monocyclic ring nor the substituted or unsubstituted fused ring, R₄₂₂₁, and R₄₂₂₂ and R₄₂₂₃ not being the single bond with L₄₂₁ and forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms; and
when n4 is 2 or 3,
a plurality of R₄₀₄ are mutually the same or different;
a plurality of R₄₂₁₁ are mutually the same or different;
a plurality of R₄₂₁₂ are mutually the same or different;
a plurality of R₄₂₁₃ are mutually the same or different;
a plurality of R₄₂₁₄ are mutually the same or different;
a plurality of R₄₂₁₅ are mutually the same or different;
a plurality of R₄₂₁₆ are mutually the same or different;
a plurality of R₄₂₁₇ are mutually the same or different; and
a plurality of R₄₂₁₈ are mutually the same or different.

In the formulae (E422) and (E423), R₉₀₁ to R₉₀₇ each independently represent the same as R₉₀₁ to R₉₀₇ in the azine derivative.

When n4 is 1 in the formula (E421), for instance, the group represented by (Ar₄₂₁)ₙ₄-L₄₂₁-* in the formula (E421) is represented by a formula (E421-1) below. In this arrangement, L₄₂₁ is a divalent linking group. * represents a bonding position with a six-membered ring in the formula (E421).

When n4 is 2 in the formula (E421), the group represented by (Ar₄₂₁)ₙ₄-L₄₂₁-* in the formula (E421) is represented by a formula (E421-2) below. Ar₄₂₁ are mutually the same or different. In this arrangement, L₄₂₁ is a trivalent linking group.

When n4 is 3 in the formula (E421), the group represented by (Ar₄₂₁)ₙ₄-L₄₂₁-* in the formula (E421) is represented by a formula (E421-3) below. Ar₄₂₁ are mutually the same or different. In this arrangement, L₄₂₁ is a tetravalent linking group.

The same applies to a group represented by (Ar₄₂₂)ₙ₄-L₄₂₁-* in the formula (E422).

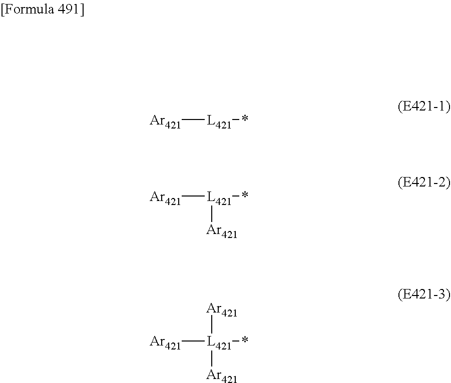

In the formulae (E421-1), (E421-2) and (E421-3), * each represent a bonding position with a six-membered ring in the formula (E421).

In the formulae (E421) and (E422), L₄₂₁ as the linking group is preferably a divalent or trivalent group derived from any of benzene, biphenyl, terphenyl, naphthalene and phenanthrene.

In an exemplary arrangement of the organic EL device according to the exemplary embodiment, the substituent for “the substituted or unsubstituted” group in the azine derivative is a group selected from the group consisting of an alkyl group having 1 to 18 carbon atoms, an aryl group having 6 to 18 ring carbon atoms and a heterocyclic group having 5 to 18 ring atoms.

In an exemplary arrangement of the organic EL device according to the exemplary embodiment, the substituent for “the substituted or unsubstituted” group in the azine derivative is an alkyl group having 1 to 5 carbon atoms.

In an exemplary arrangement of the organic EL device according to the exemplary embodiment, the groups specified to be “substituted or unsubstituted” in the azine derivative are each an “unsubstituted” group.

### Benzimidazole Derivative

In an exemplary arrangement of the organic EL device according to the exemplary embodiment, at least one electron transporting layer in the electron transporting zone contains, as a nitrogen-containing compound, a benzimidazole derivative.

In an exemplary arrangement of the organic EL device according to the exemplary embodiment, the benzimidazole derivative contained in the electron transporting layer is a compound represented by a formula (E41) below.

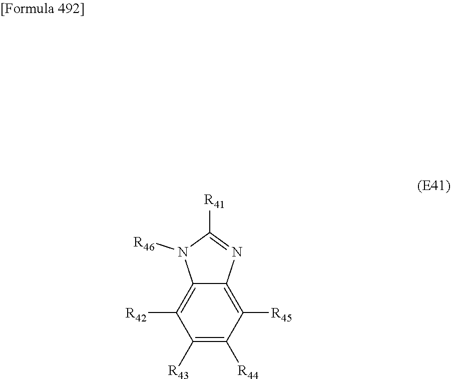

In the formula (E41):
at least one combination of adjacent two or more of R₄₁ to R₄₆ are mutually bonded to form a substituted or unsubstituted monocyclic ring, mutually bonded to form a substituted or unsubstituted fused ring, or not mutually bonded; and
R₄₁ to R₄₆ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In the formula (E41), R₉₀₁ to R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different; and
when a plurality of R₉₀₇ are present, the plurality of R₉₀₇ are mutually the same or different.

In an exemplary arrangement of the organic EL device according to the exemplary embodiment, the benzimidazole derivative contained in the electron transporting layer is a compound represented by a formula (E41A), (E41B), (E41C), (E41D) or (E41E) below.

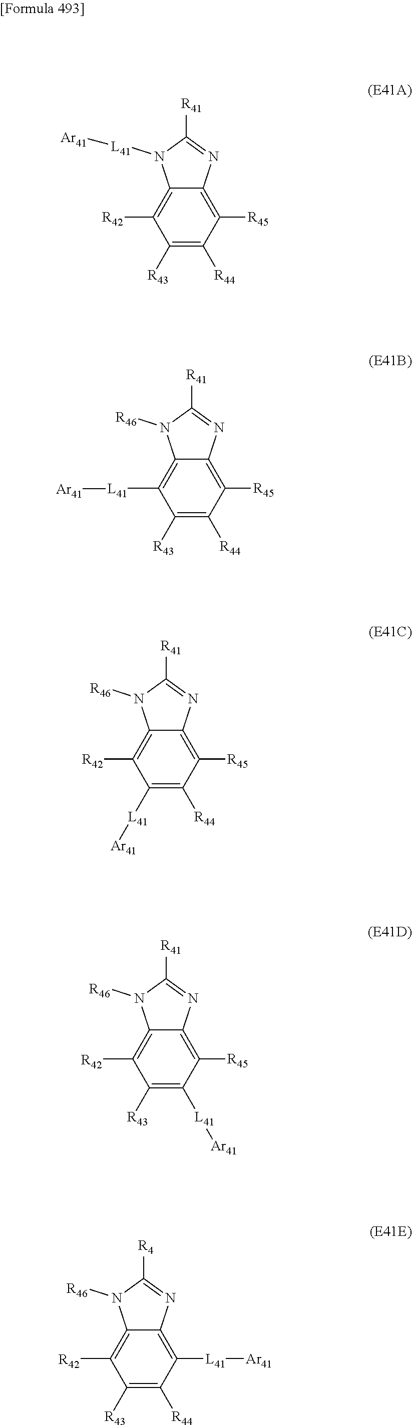

In the formulae (E41A), (E41B), (E41C), (E41 D) and (E41E): R₄₁ to R₄₆ each independently represent the same as R₄₁ to R₄₆ in the formula (E41);
L₄₁ is a single bond, a substituted or unsubstituted arylene group having 6 to 50 ring carbon atoms, or a substituted or unsubstituted divalent heterocyclic group having 5 to 50 ring atoms;
A₄₁ is a substituted or unsubstituted alkyl group having 1 to 50 carbon atoms, a group represented by —O—(R₉₀₄), a substituted or unsubstituted aryl group having 6 to 50 ring carbon atoms, or a substituted or unsubstituted heterocyclic group having 5 to 50 ring atoms; and
R₉₀₄ represents the same as R₉₀₄ in the benzimidazole derivative.

In an exemplary arrangement of the organic EL device of the exemplary embodiment, Ar₄₁ in the formulae (E41A), (E41B), (E41C), (E41D) and (E41E) are each a group represented by a formula (E412) below.

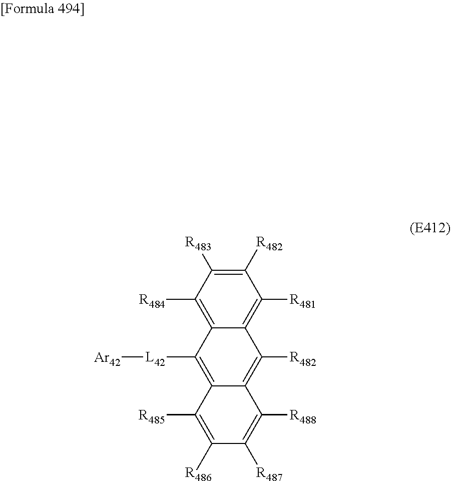

In the formula (E412):
one of R₄₈₁ to R₄₈₉ is a single bond with L₄₁;
at least one combination of adjacent two or more of R₄₈₁ to R₄₈₉ not being the single bond with L₄₁ are mutually bonded to form a substituted or unsubstituted monocyclic ring, mutually bonded to form a substituted or unsubstituted fused ring, or not mutually bonded;
R₄₈₁ to R₄₈₉ not being the single bond with L₄₁, not forming the substituted or unsubstituted monocyclic ring, and not forming the substituted or unsubstituted fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms;
L₄₂ is each independently a single bond, a substituted or unsubstituted arylene group having 6 to 50 ring carbon atoms, or a substituted or unsubstituted divalent heterocyclic group having 5 to 50 ring atoms; and
Ar₄₂ is each independently a substituted or unsubstituted aryl group having 6 to 50 ring carbon atoms, or a substituted or unsubstituted heterocyclic group having 5 to 50 ring atoms.

In the formula (E412), R₉₀₁ to R₉₀₇ each independently represent the same as R₉₀₁ to R₉₀₇ in the benzimidazole derivative.

In the formulae (E41A), (E41B), (E41C), (E41 D) and (E41E): L₄₁ is preferably a substituted or unsubstituted phenylene group, a substituted or unsubstituted biphenylene group, a substituted or unsubstituted naphthylene group, a substituted or unsubstituted pyridinylene group, or a substituted or unsubstituted 9,9′-spirobifluorenylene group.

In the formulae (E41A), (E41B), (E41C), (E41D) and (E41E): L₄₁ is more preferably an unsubstituted phenylene group, an unsubstituted biphenylene group, an unsubstituted naphthylene group, an unsubstituted pyridinylene group, or an unsubstituted 9,9′-spirobifluorenylene group.

In the formulae (E41A), (E41B), (E41C), (E41D) and (E41E): Ar₄₁ is preferably a substituted or unsubstituted phenyl group, a substituted or unsubstituted biphenyl group, a substituted or unsubstituted terphenyl group, a substituted or unsubstituted naphthyl group, a substituted or unsubstituted phenanthryl group, a substituted or unsubstituted fluoranthenyl group, a substituted or unsubstituted pyrenyl group, or a substituted or unsubstituted fluorenyl group.

In the formulae (E41A), (E41B), (E41C), (E41D) and (E41E): Ar₄₁ is more preferably an unsubstituted phenyl group, an unsubstituted biphenyl group, an unsubstituted terphenyl group, an unsubstituted naphthyl group, an unsubstituted phenanthryl group, an unsubstituted fluoranthenyl group, an unsubstituted pyrenyl group, or an unsubstituted 9,9-dimethylfluorenyl group.

In an exemplary arrangement of the organic EL device according to the exemplary embodiment, the benzimidazole derivative is a compound represented by a formula (E413) below.

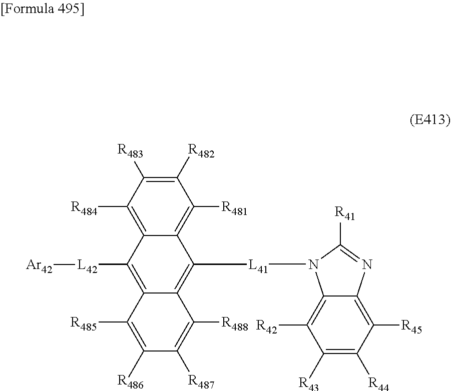

In the formula (E413):
R₄₁ to R₄₅ and L₄₁ each independently represent the same as R₄₁ to R₄₅ and L₄₁ in the formula (E41A); and
R₄₈₁ to R₄₈₈, L₄₂ and Ar₄₂ each independently represent the same as R₄₈₁ to R₄₈₈, L₄₂ and Ar₄₂ in the formula (E412).

In an exemplary arrangement of the organic EL device according to the exemplary embodiment, the groups specified to be “substituted or unsubstituted” in the benzimidazole derivative are each an “unsubstituted” group.

### Specific Examples of Nitrogen-Containing Compound

Specific examples of the nitrogen-containing compound include the following compounds. However, the invention is by no means limited to the specifically listed nitrogen-containing compounds.

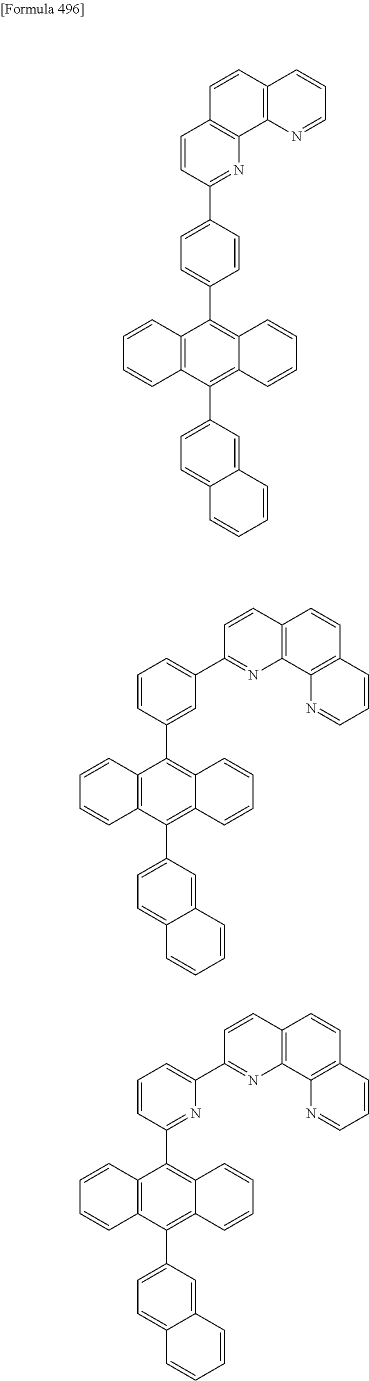 [SMILES: c1ccc2cc(-c3c4ccccc4c(-c4ccc(-c5ccc6ccc7cccnc7c6n5)cc4)c4ccccc34)ccc2c1 ; c1cc(-c2ccc3ccc4cccnc4c3n2)cc(-c2c3ccccc3c(-c3ccc4ccccc4c3)c3ccccc23)c1 ; c1cc(-c2ccc3ccc4cccnc4c3n2)nc(-c2c3ccccc3c(-c3ccc4ccccc4c3)c3ccccc23)c1]

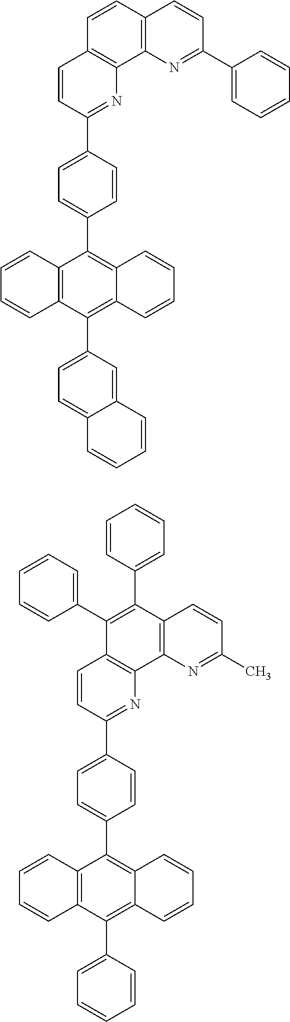

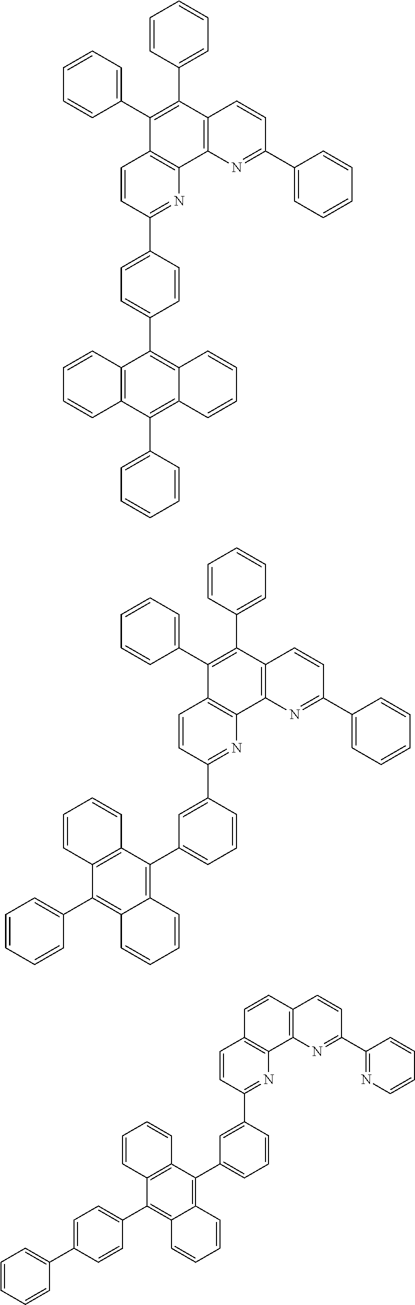 [SMILES: c1ccc(-c2ccc3c(-c4ccccc4)c(-c4ccccc4)c4ccc(-c5ccc(-c6c7ccccc7c(-c7ccccc7)c7ccccc67)cc5)nc4c3n2)cc1 ; c1ccc(-c2ccc3c(-c4ccccc4)c(-c4ccccc4)c4ccc(-c5cccc(-c6c7ccccc7c(-c7ccccc7)c7ccccc67)c5)nc4c3n2)cc1 ; c1ccc(-c2ccc(-c3c4ccccc4c(-c4cccc(-c5ccc6ccc7ccc(-c8ccccn8)nc7c6n5)c4)c4ccccc34)cc2)cc1]

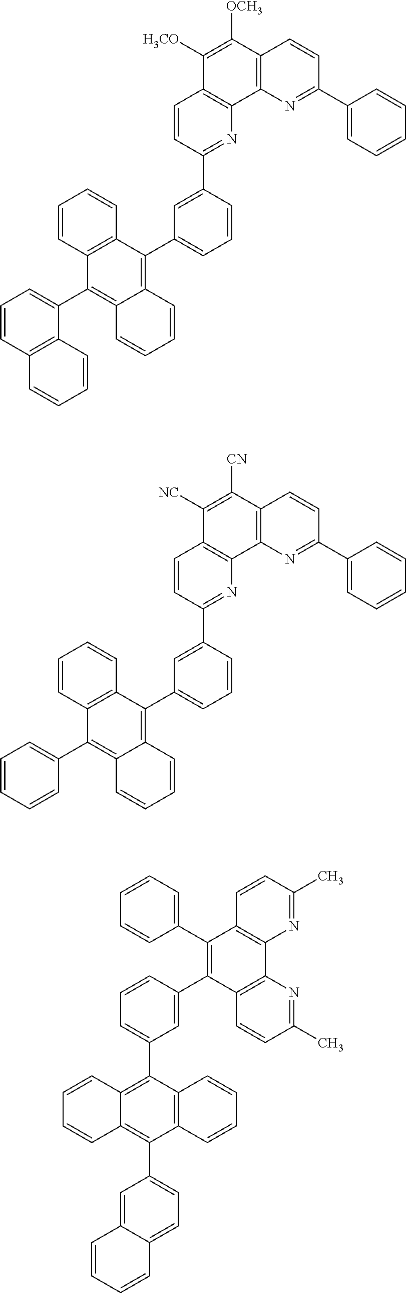 [SMILES: COc1c(OC)c2ccc(-c3cccc(-c4c5ccccc5c(-c5cccc6ccccc56)c5ccccc45)c3)nc2c2nc(-c3ccccc3)ccc12 ; N#Cc1c(C#N)c2ccc(-c3cccc(-c4c5ccccc5c(-c5ccccc5)c5ccccc45)c3)nc2c2nc(-c3ccccc3)ccc12 ; Cc1ccc2c(-c3ccccc3)c(-c3cccc(-c4c5ccccc5c(-c5ccc6ccccc6c5)c5ccccc45)c3)c3ccc(C)nc3c2n1]

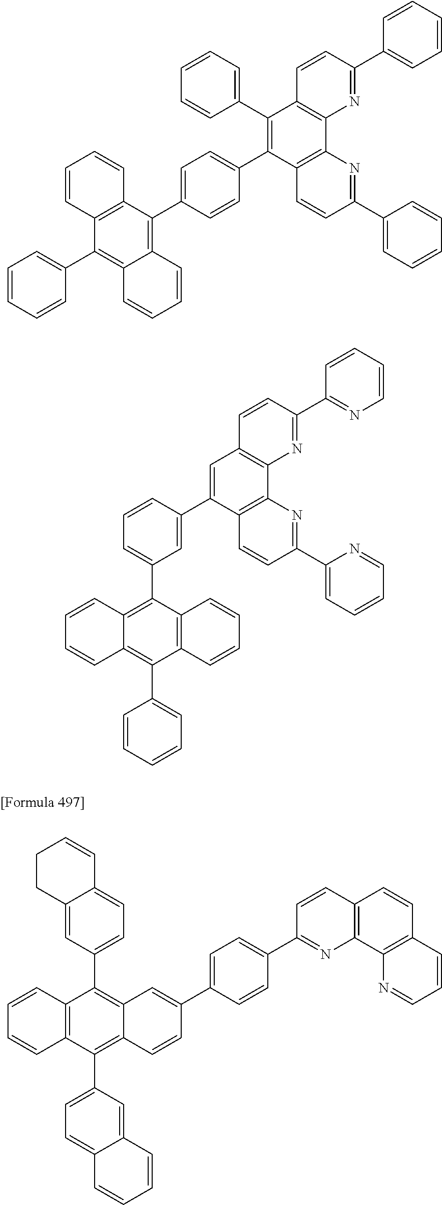 [SMILES: c1ccc(-c2ccc3c(-c4ccccc4)c(-c4ccc(-c5c6ccccc6c(-c6ccccc6)c6ccccc56)cc4)c4ccc(-c5ccccc5)nc4c3n2)cc1 ; c1ccc(-c2c3ccccc3c(-c3cccc(-c4cc5ccc(-c6ccccn6)nc5c5nc(-c6ccccn6)ccc45)c3)c3ccccc23)cc1 ; C1=Cc2ccc(-c3c4ccccc4c(-c4ccc5ccccc5c4)c4ccc(-c5ccc(-c6ccc7ccc8cccnc8c7n6)cc5)cc34)cc2CC1]

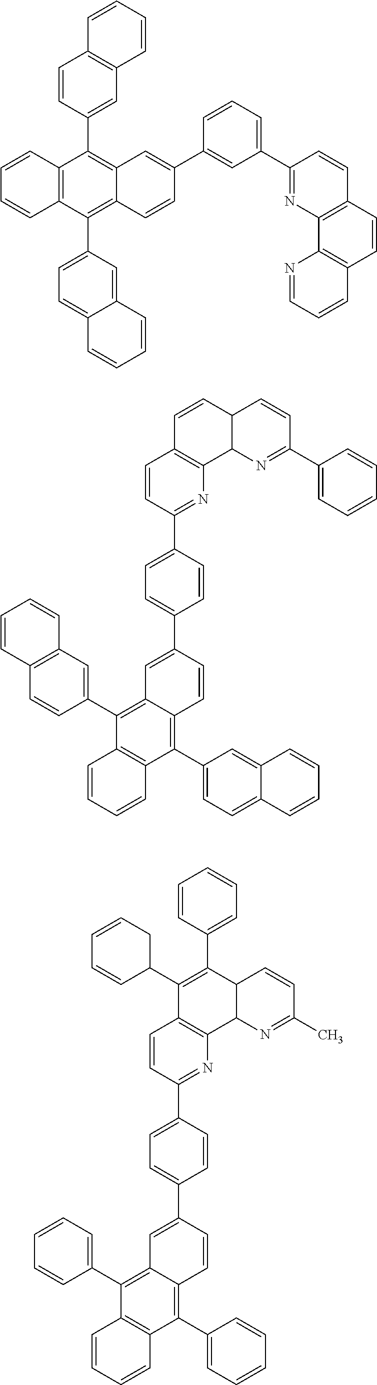 [SMILES: c1cc(-c2ccc3c(-c4ccc5ccccc5c4)c4ccccc4c(-c4ccc5ccccc5c4)c3c2)cc(-c2ccc3ccc4cccnc4c3n2)c1 ; C1=CC2C=Cc3ccc(-c4ccc(-c5ccc6c(-c7ccc8ccccc8c7)c7ccccc7c(-c7ccc8ccccc8c7)c6c5)cc4)nc3C2N=C1c1ccccc1 ; CC1=NC2c3nc(-c4ccc(-c5ccc6c(-c7ccccc7)c7ccccc7c(-c7ccccc7)c6c5)cc4)ccc3C(C3C=CC=CC3)=C(c3ccccc3)C2C=C1]

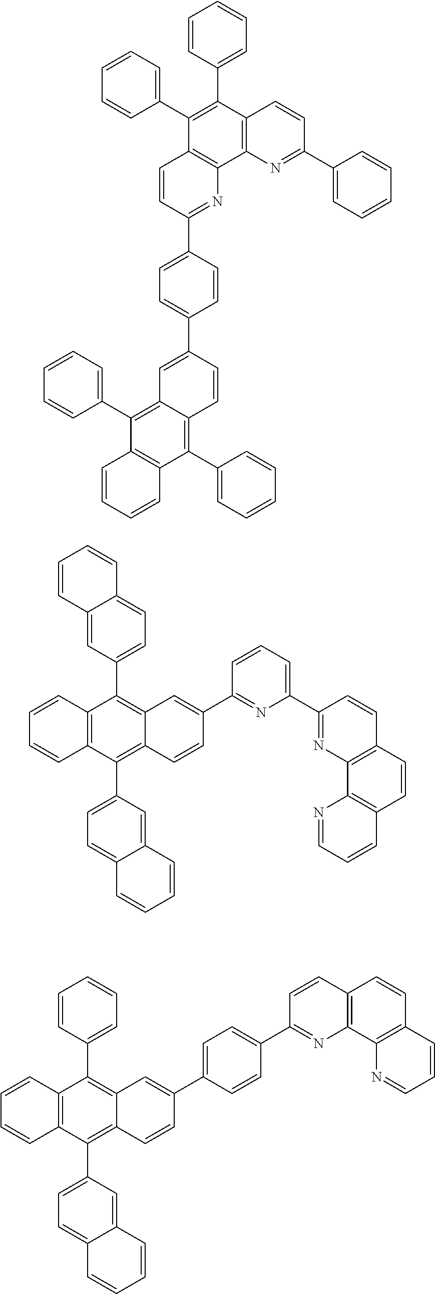

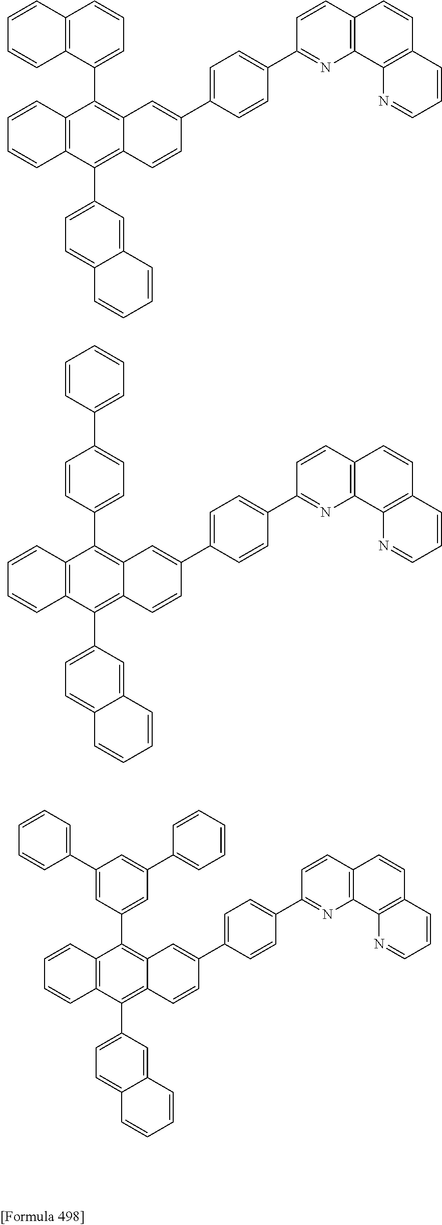

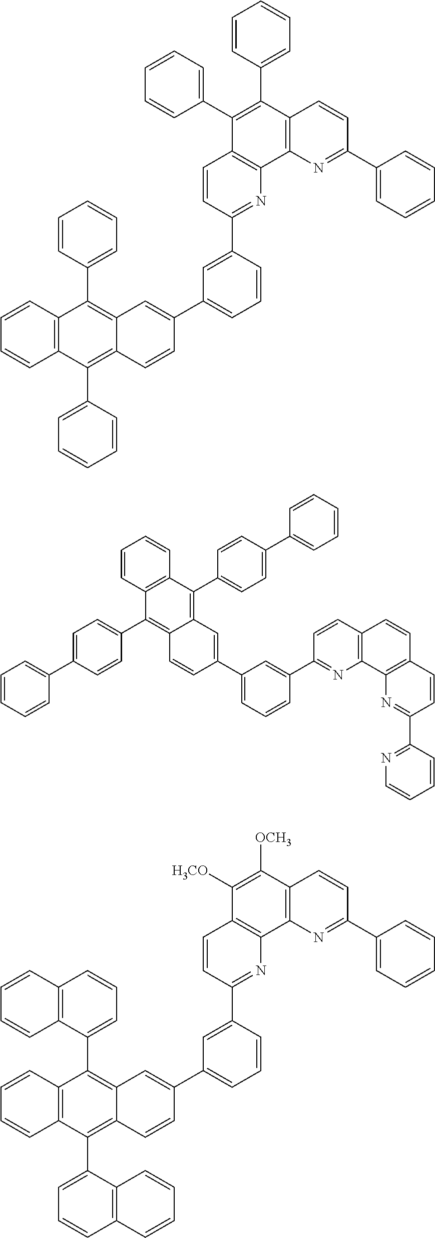 [SMILES: c1ccc(-c2ccc3c(-c4ccccc4)c(-c4ccccc4)c4ccc(-c5cccc(-c6ccc7c(-c8ccccc8)c8ccccc8c(-c8ccccc8)c7c6)c5)nc4c3n2)cc1 ; c1ccc(-c2ccc(-c3c4ccccc4c(-c4ccc(-c5ccccc5)cc4)c4cc(-c5cccc(-c6ccc7ccc8ccc(-c9ccccn9)nc8c7n6)c5)ccc34)cc2)cc1 ; COc1c(OC)c2ccc(-c3cccc(-c4ccc5c(-c6cccc7ccccc67)c6ccccc6c(-c6cccc7ccccc67)c5c4)c3)nc2c2nc(-c3ccccc3)ccc12]

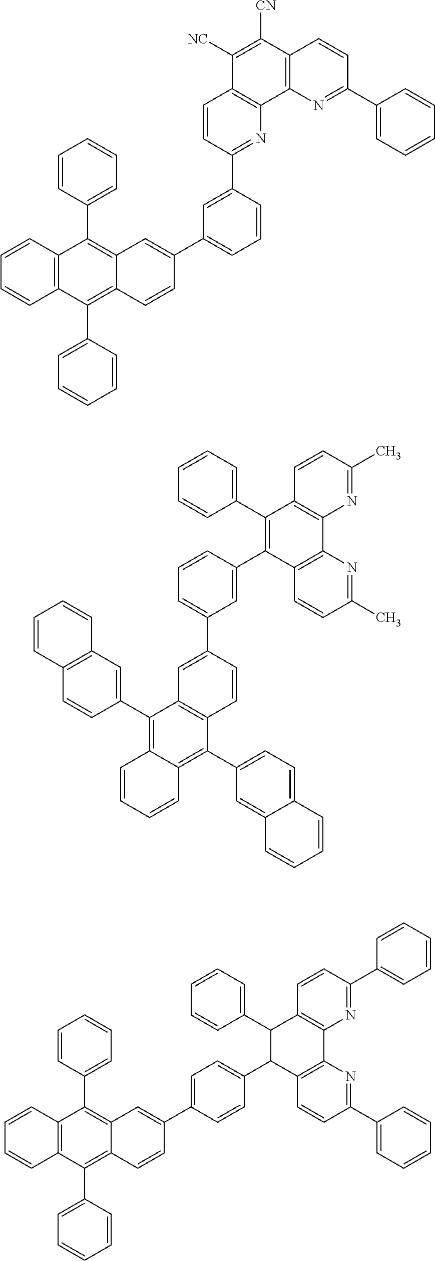 [SMILES: N#Cc1c(C#N)c2ccc(-c3cccc(-c4ccc5c(-c6ccccc6)c6ccccc6c(-c6ccccc6)c5c4)c3)nc2c2nc(-c3ccccc3)ccc12 ; Cc1ccc2c(-c3ccccc3)c(-c3cccc(-c4ccc5c(-c6ccc7ccccc7c6)c6ccccc6c(-c6ccc7ccccc7c6)c5c4)c3)c3ccc(C)nc3c2n1 ; c1ccc(-c2ccc3c(n2)-c2nc(-c4ccccc4)ccc2C(c2ccc(-c4ccc5c(-c6ccccc6)c6ccccc6c(-c6ccccc6)c5c4)cc2)C3c2ccccc2)cc1]

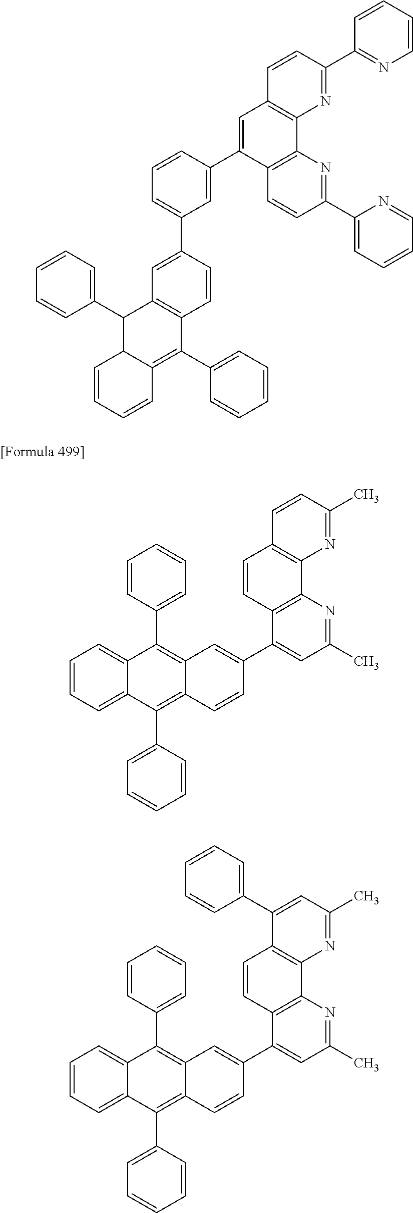 [SMILES: C1=CC2=C(c3ccccc3)c3ccc(-c4cccc(-c5cc6ccc(-c7ccccn7)nc6c6nc(-c7ccccn7)ccc56)c4)cc3C(c3ccccc3)C2C=C1 ; Cc1ccc2ccc3c(-c4ccc5c(-c6ccccc6)c6ccccc6c(-c6ccccc6)c5c4)cc(C)nc3c2n1 ; Cc1cc(-c2ccccc2)c2ccc3c(-c4ccc5c(-c6ccccc6)c6ccccc6c(-c6ccccc6)c5c4)cc(C)nc3c2n1]

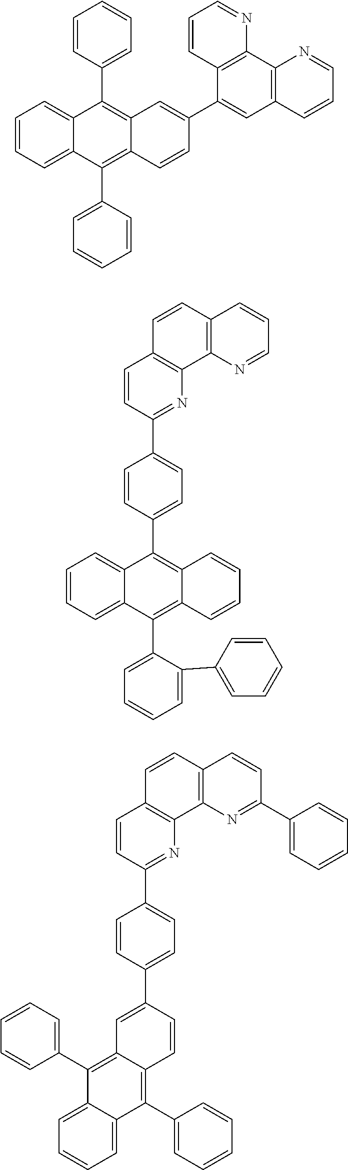

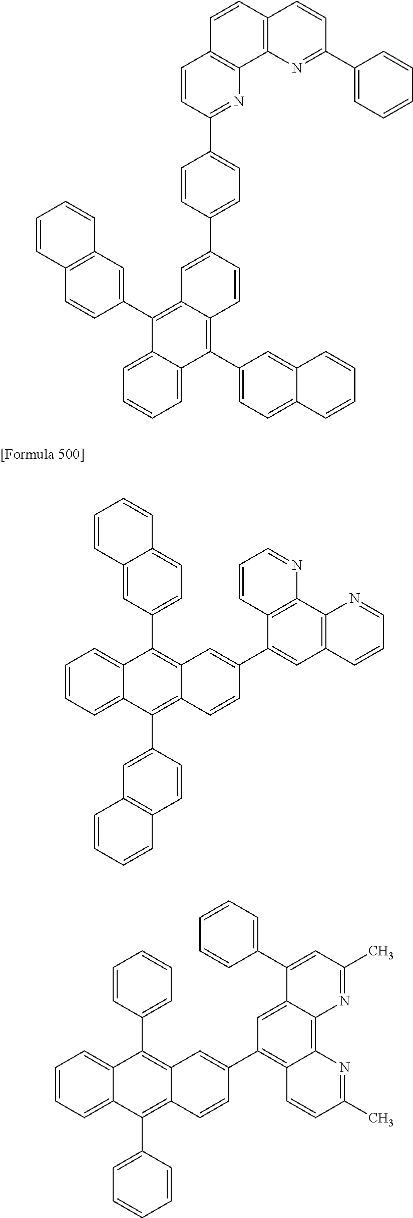 [SMILES: c1ccc(-c2ccc3ccc4ccc(-c5ccc(-c6ccc7c(-c8ccc9ccccc9c8)c8ccccc8c(-c8ccc9ccccc9c8)c7c6)cc5)nc4c3n2)cc1 ; c1ccc2cc(-c3c4ccccc4c(-c4ccc5ccccc5c4)c4cc(-c5cc6cccnc6c6ncccc56)ccc34)ccc2c1 ; Cc1ccc2c(-c3ccc4c(-c5ccccc5)c5ccccc5c(-c5ccccc5)c4c3)cc3c(-c4ccccc4)cc(C)nc3c2n1]

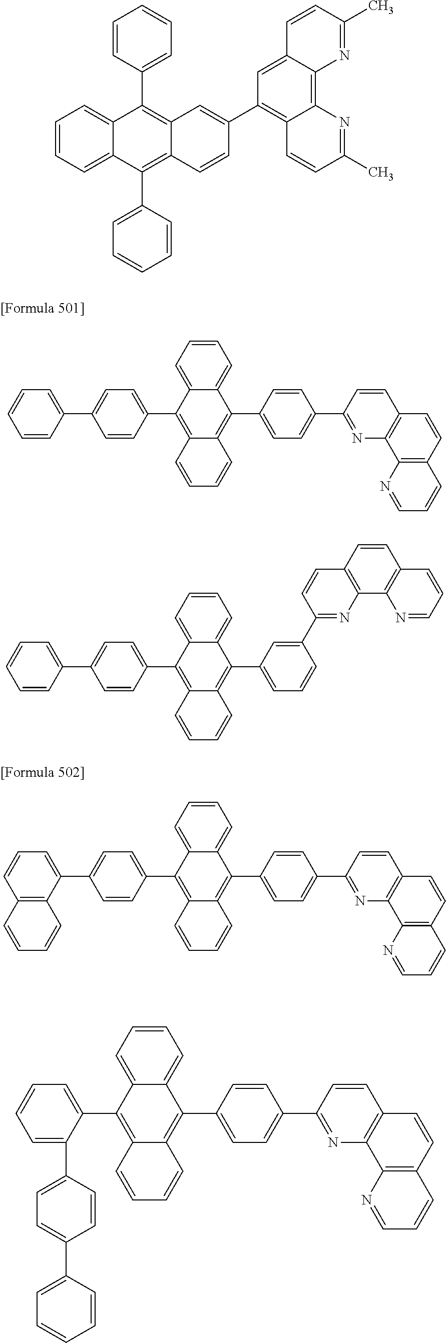

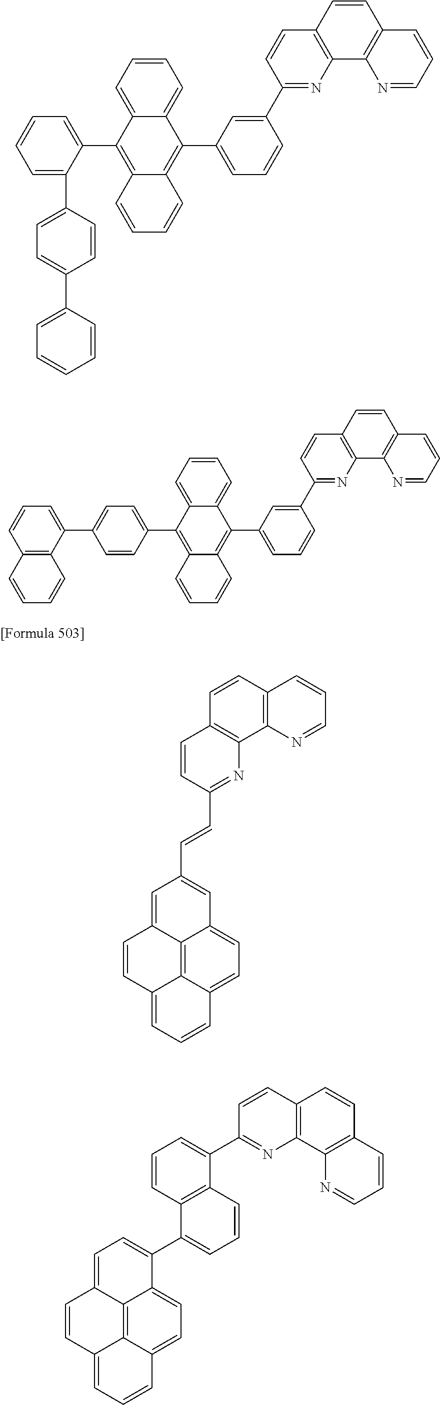 [SMILES: c1ccc(-c2ccc(-c3ccccc3-c3c4ccccc4c(-c4cccc(-c5ccc6ccc7cccnc7c6n5)c4)c4ccccc34)cc2)cc1 ; c1cc(-c2ccc3ccc4cccnc4c3n2)cc(-c2c3ccccc3c(-c3ccc(-c4cccc5ccccc45)cc3)c3ccccc23)c1 ; C(=C\c1ccc2ccc3cccnc3c2n1)/c1cc2ccc3cccc4ccc(c1)c2c34 ; c1cnc2c(c1)ccc1ccc(-c3cccc4c(-c5ccc6ccc7cccc8ccc5c6c78)cccc34)nc12]

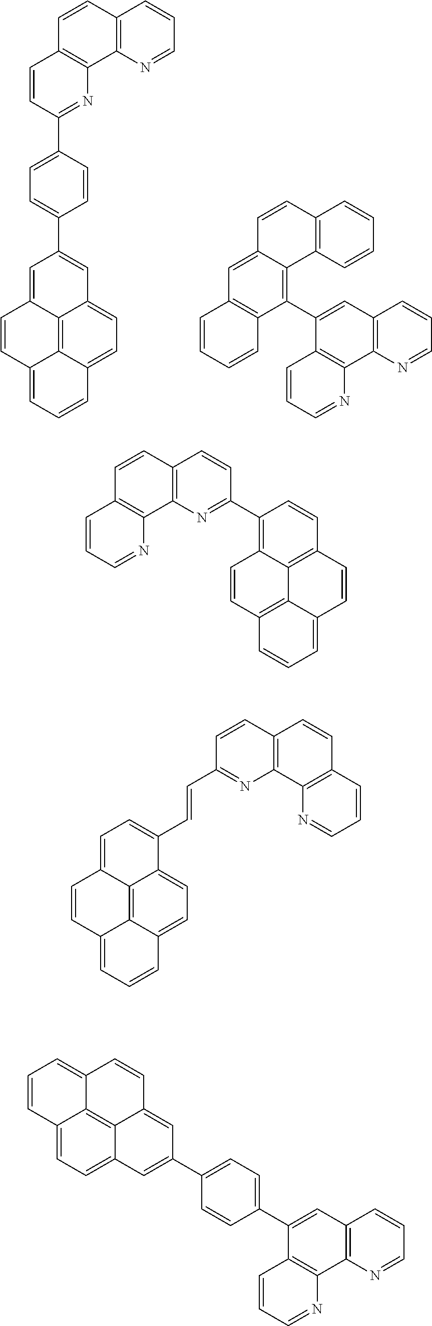 [SMILES: c1cnc2c(c1)ccc1ccc(-c3ccc(-c4cc5ccc6cccc7ccc(c4)c5c67)cc3)nc12 ; c1ccc2c(-c3cc4cccnc4c4ncccc34)c3c(ccc4ccccc43)cc2c1 ; c1cnc2c(c1)ccc1ccc(-c3ccc4ccc5cccc6ccc3c4c56)nc12 ; C(=C\c1ccc2ccc3cccc4ccc1c2c34)/c1ccc2ccc3cccnc3c2n1 ; c1cnc2c(c1)cc(-c1ccc(-c3cc4ccc5cccc6ccc(c3)c4c56)cc1)c1cccnc12]

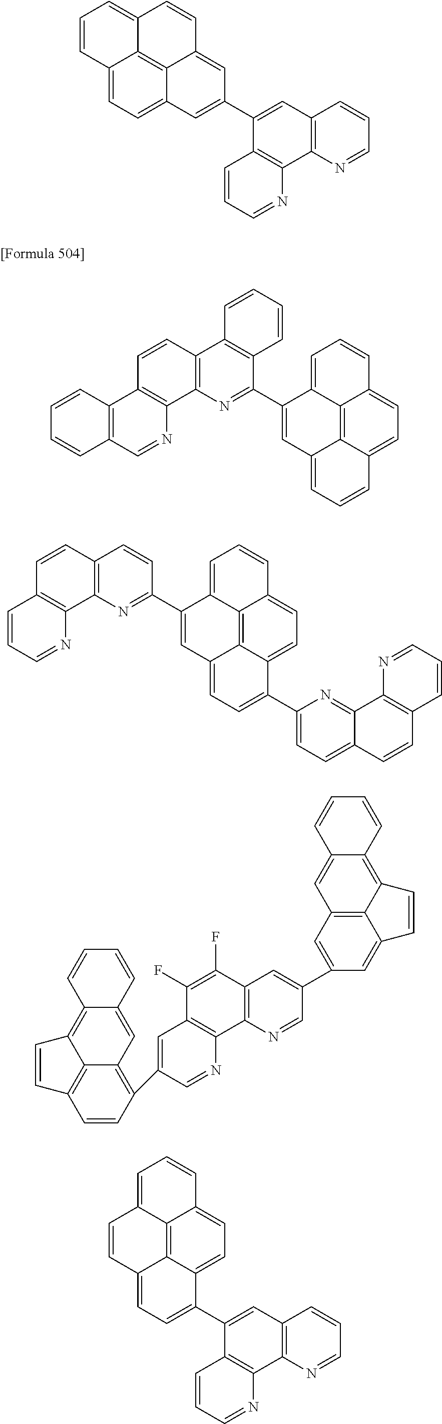

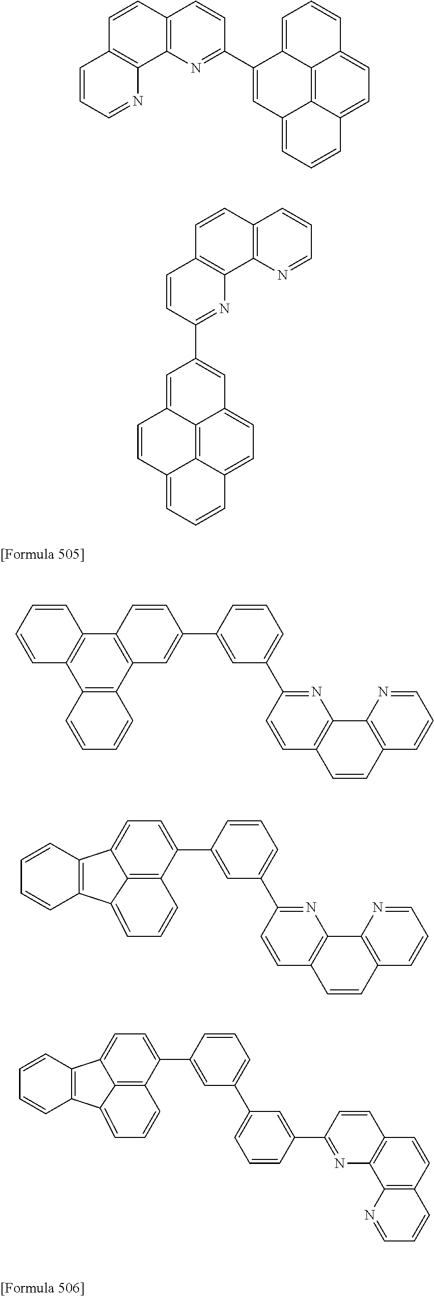 [SMILES: c1cnc2c(c1)ccc1ccc(-c3cc4cccc5ccc6cccc3c6c54)nc12 ; c1cnc2c(c1)ccc1ccc(-c3cc4ccc5cccc6ccc(c3)c4c56)nc12 ; c1cc(-c2ccc3c4ccccc4c4ccccc4c3c2)cc(-c2ccc3ccc4cccnc4c3n2)c1 ; c1cc(-c2ccc3ccc4cccnc4c3n2)cc(-c2ccc3c4c(cccc24)-c2ccccc2-3)c1 ; c1cc(-c2cccc(-c3ccc4c5c(cccc35)-c3ccccc3-4)c2)cc(-c2ccc3ccc4cccnc4c3n2)c1]

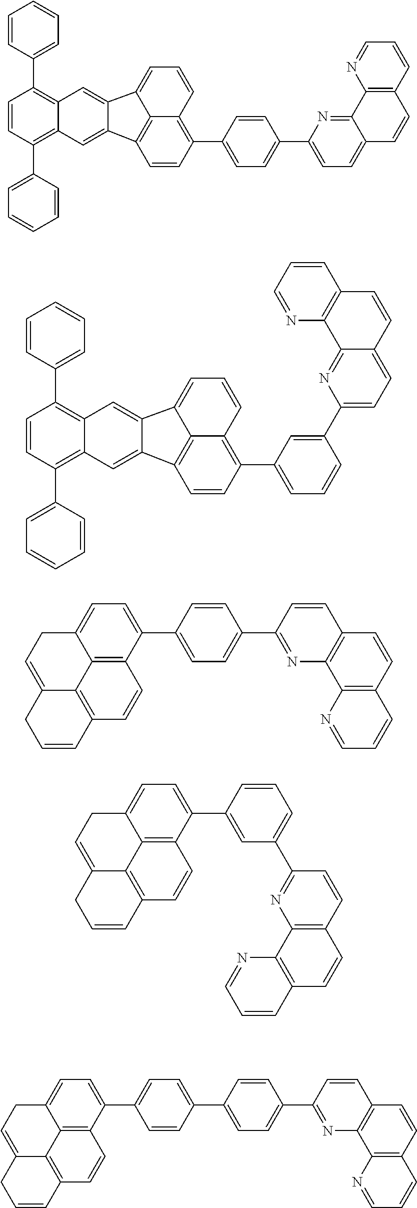 [SMILES: c1ccc(-c2ccc(-c3ccccc3)c3cc4c(cc23)-c2cccc3c(-c5ccc(-c6ccc7ccc8cccnc8c7n6)cc5)ccc-4c23)cc1 ; c1ccc(-c2ccc(-c3ccccc3)c3cc4c(cc23)-c2cccc3c(-c5cccc(-c6ccc7ccc8cccnc8c7n6)c5)ccc-4c23)cc1 ; C1=Cc2ccc3c(-c4ccc(-c5ccc6ccc7cccnc7c6n5)cc4)ccc4c3c2C(=CC4)C1 ; C1=Cc2ccc3c(-c4cccc(-c5ccc6ccc7cccnc7c6n5)c4)ccc4c3c2C(=CC4)C1 ; C1=Cc2ccc3c(-c4ccc(-c5ccc(-c6ccc7ccc8cccnc8c7n6)cc5)cc4)ccc4c3c2C(=CC4)C1]

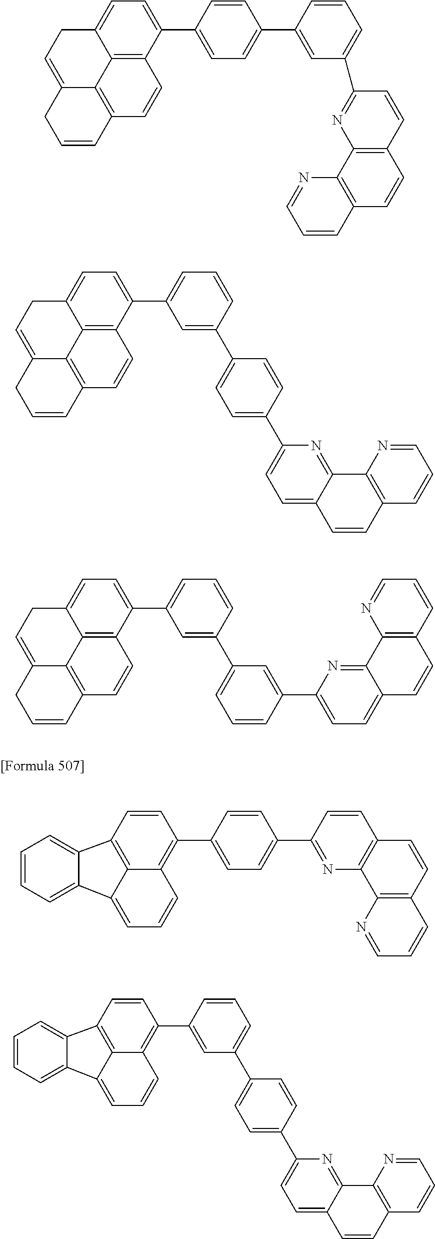 [SMILES: C1=Cc2ccc3c(-c4ccc(-c5cccc(-c6ccc7ccc8cccnc8c7n6)c5)cc4)ccc4c3c2C(=CC4)C1 ; C1=Cc2ccc3c(-c4cccc(-c5ccc(-c6ccc7ccc8cccnc8c7n6)cc5)c4)ccc4c3c2C(=CC4)C1 ; C1=Cc2ccc3c(-c4cccc(-c5cccc(-c6ccc7ccc8cccnc8c7n6)c5)c4)ccc4c3c2C(=CC4)C1 ; c1ccc2c(c1)-c1cccc3c(-c4ccc(-c5ccc6ccc7cccnc7c6n5)cc4)ccc-2c13 ; c1cc(-c2ccc(-c3ccc4ccc5cccnc5c4n3)cc2)cc(-c2ccc3c4c(cccc24)-c2ccccc2-3)c1]

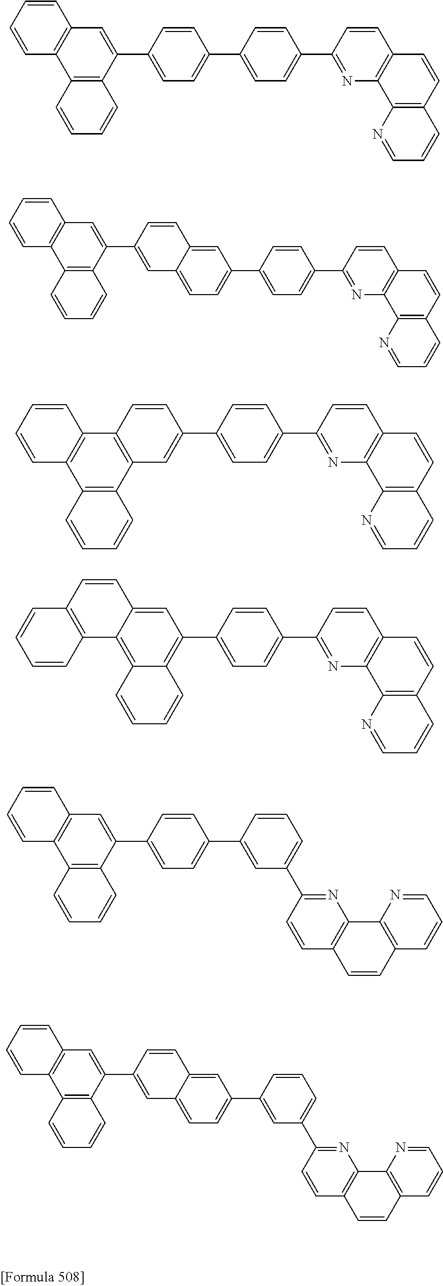

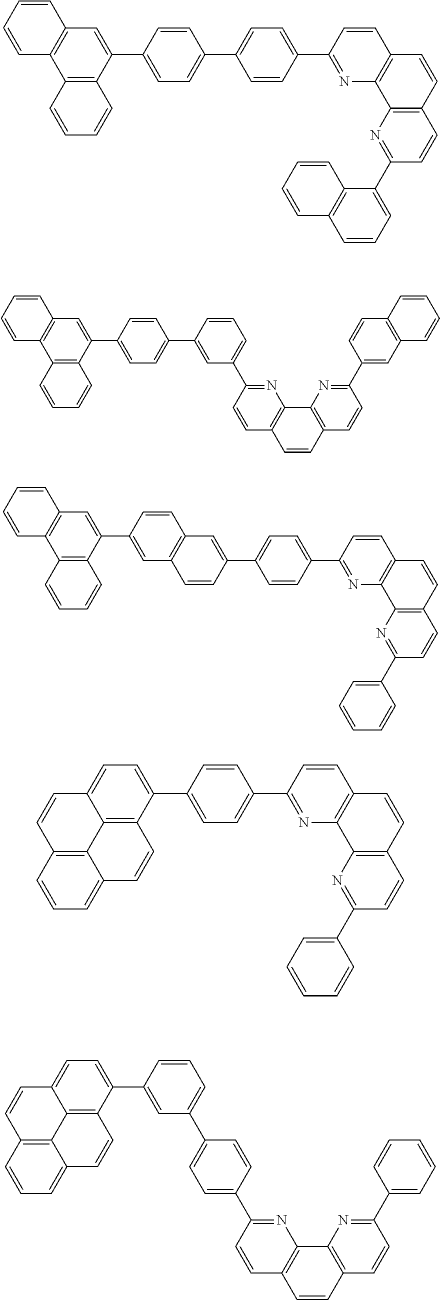

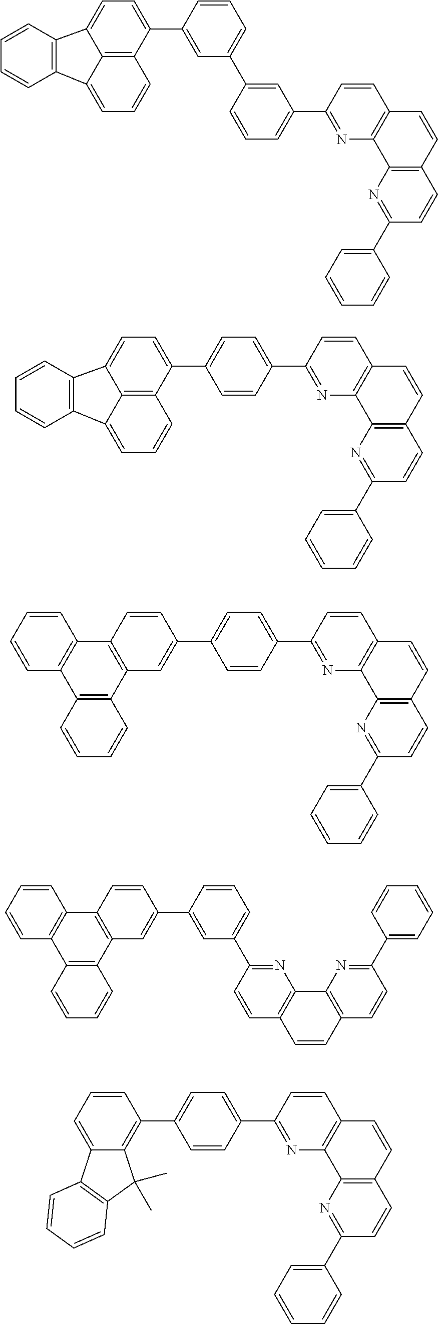

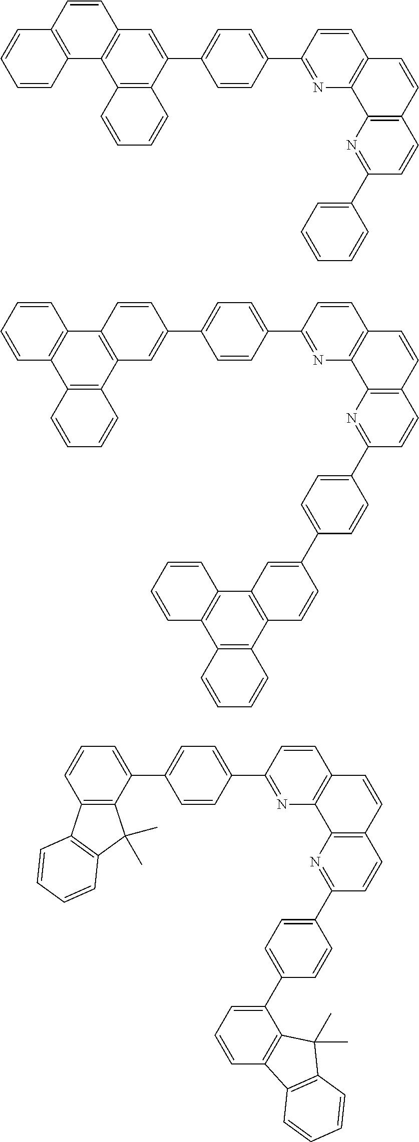

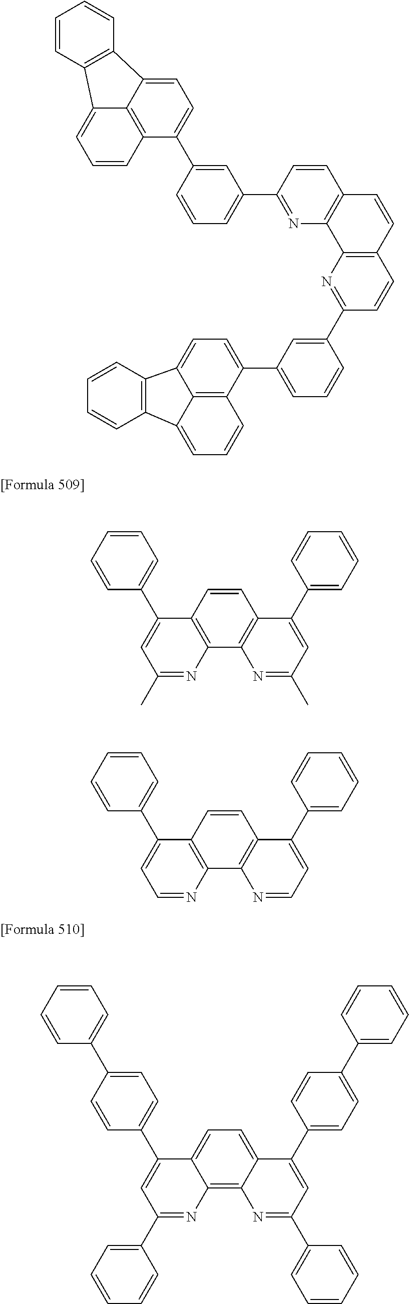 [SMILES: c1cc(-c2ccc3ccc4ccc(-c5cccc(-c6ccc7c8c(cccc68)-c6ccccc6-7)c5)nc4c3n2)cc(-c2ccc3c4c(cccc24)-c2ccccc2-3)c1 ; Cc1cc(-c2ccccc2)c2ccc3c(-c4ccccc4)cc(C)nc3c2n1 ; c1ccc(-c2ccnc3c2ccc2c(-c4ccccc4)ccnc23)cc1 ; c1ccc(-c2ccc(-c3cc(-c4ccccc4)nc4c3ccc3c(-c5ccc(-c6ccccc6)cc5)cc(-c5ccccc5)nc34)cc2)cc1]

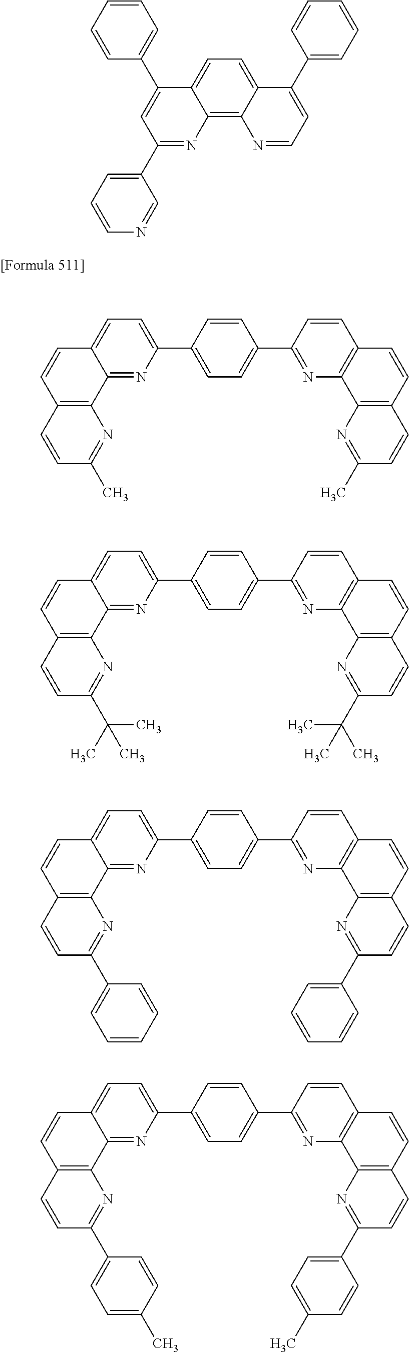

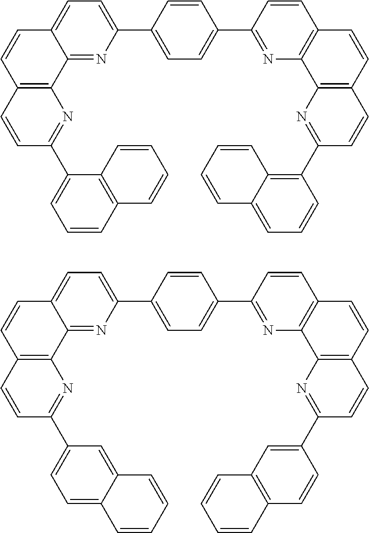

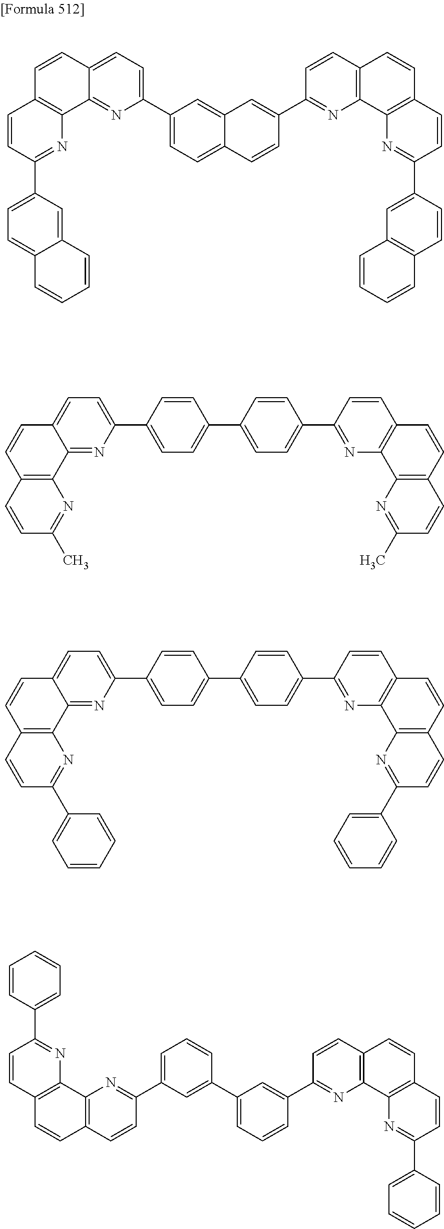 [SMILES: c1ccc2cc(-c3ccc4ccc5ccc(-c6ccc7ccc(-c8ccc9ccc%10ccc(-c%11ccc%12ccccc%12c%11)nc%10c9n8)cc7c6)nc5c4n3)ccc2c1 ; Cc1ccc2ccc3ccc(-c4ccc(-c5ccc(-c6ccc7ccc8ccc(C)nc8c7n6)cc5)cc4)nc3c2n1 ; c1ccc(-c2ccc3ccc4ccc(-c5ccc(-c6ccc(-c7ccc8ccc9ccc(-c%10ccccc%10)nc9c8n7)cc6)cc5)nc4c3n2)cc1 ; c1ccc(-c2ccc3ccc4ccc(-c5cccc(-c6cccc(-c7ccc8ccc9ccc(-c%10ccccc%10)nc9c8n7)c6)c5)nc4c3n2)cc1]

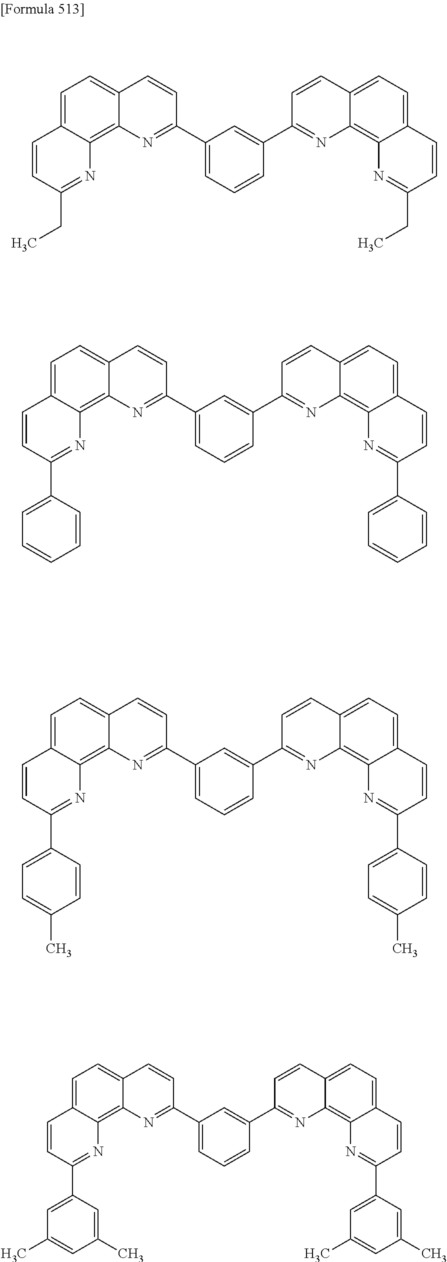

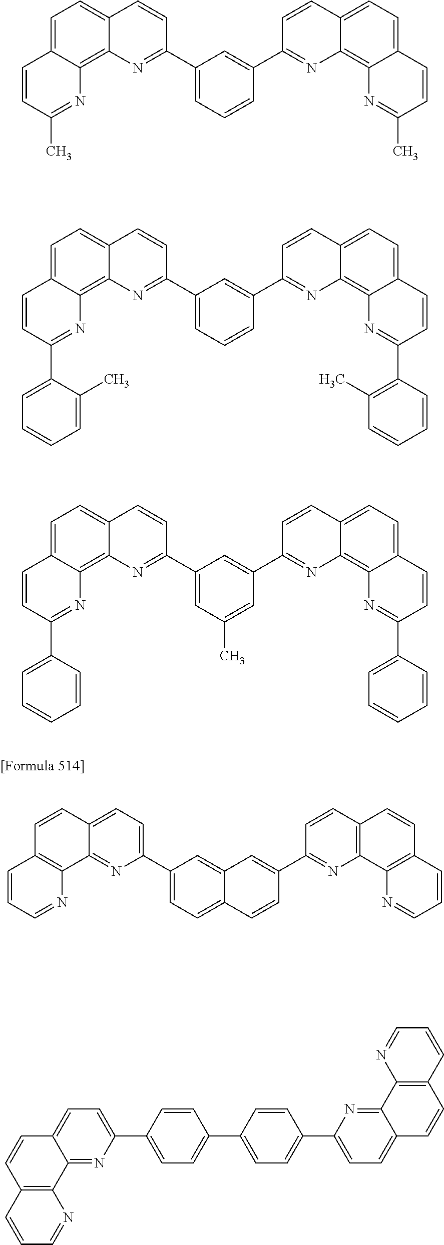 [SMILES: Cc1ccc2ccc3ccc(-c4cccc(-c5ccc6ccc7ccc(C)nc7c6n5)c4)nc3c2n1 ; Cc1ccccc1-c1ccc2ccc3ccc(-c4cccc(-c5ccc6ccc7ccc(-c8ccccc8C)nc7c6n5)c4)nc3c2n1 ; Cc1cc(-c2ccc3ccc4ccc(-c5ccccc5)nc4c3n2)cc(-c2ccc3ccc4ccc(-c5ccccc5)nc4c3n2)c1 ; c1cnc2c(c1)ccc1ccc(-c3ccc4ccc(-c5ccc6ccc7cccnc7c6n5)cc4c3)nc12 ; c1cnc2c(c1)ccc1ccc(-c3ccc(-c4ccc(-c5ccc6ccc7cccnc7c6n5)cc4)cc3)nc12]

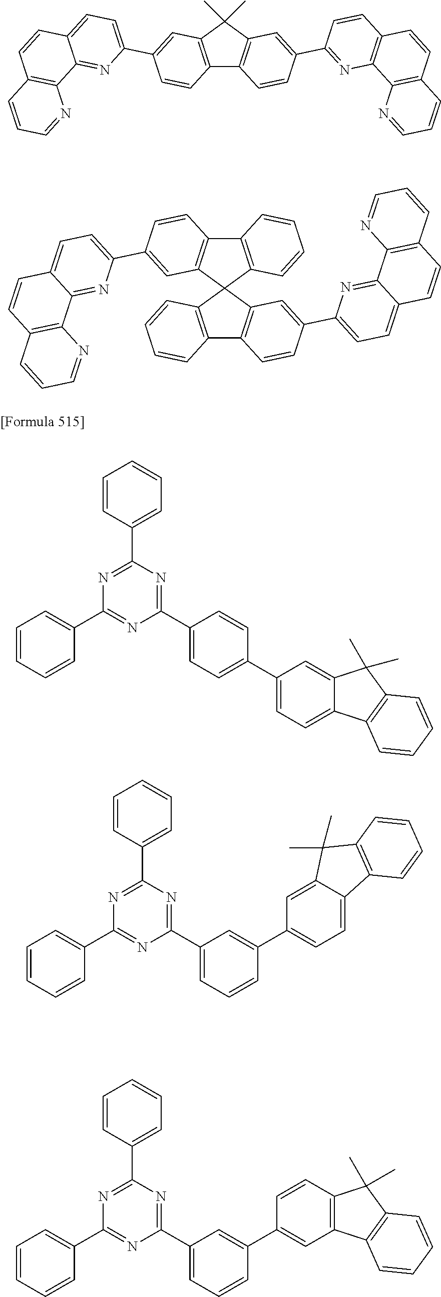 [SMILES: CC1(C)c2cc(-c3ccc4ccc5cccnc5c4n3)ccc2-c2ccc(-c3ccc4ccc5cccnc5c4n3)cc21 ; c1ccc2c(c1)-c1ccc(-c3ccc4ccc5cccnc5c4n3)cc1C21c2ccccc2-c2ccc(-c3ccc4ccc5cccnc5c4n3)cc21 ; CC1(C)c2ccccc2-c2ccc(-c3ccc(-c4nc(-c5ccccc5)nc(-c5ccccc5)n4)cc3)cc21 ; CC1(C)c2ccccc2-c2ccc(-c3cccc(-c4nc(-c5ccccc5)nc(-c5ccccc5)n4)c3)cc21 ; CC1(C)c2ccccc2-c2cc(-c3cccc(-c4nc(-c5ccccc5)nc(-c5ccccc5)n4)c3)ccc21]

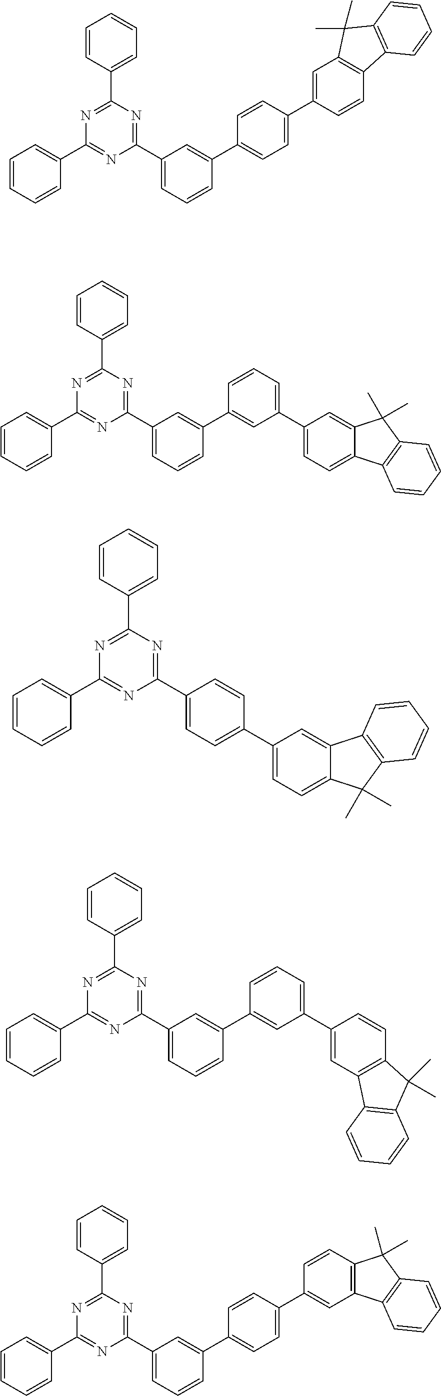

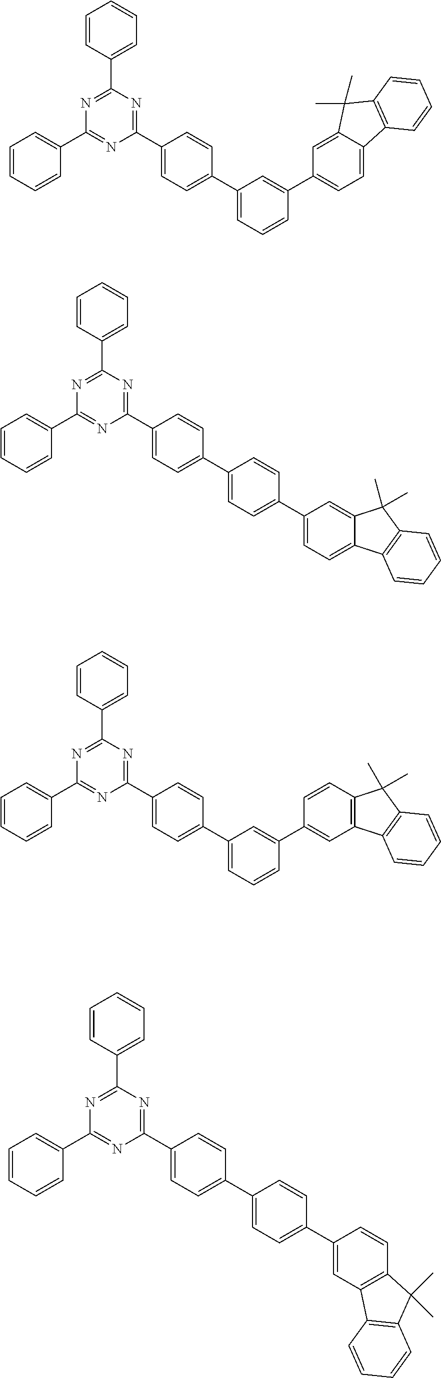 [SMILES: CC1(C)c2ccccc2-c2ccc(-c3cccc(-c4ccc(-c5nc(-c6ccccc6)nc(-c6ccccc6)n5)cc4)c3)cc21 ; CC1(C)c2ccccc2-c2ccc(-c3ccc(-c4ccc(-c5nc(-c6ccccc6)nc(-c6ccccc6)n5)cc4)cc3)cc21 ; CC1(C)c2ccccc2-c2cc(-c3cccc(-c4ccc(-c5nc(-c6ccccc6)nc(-c6ccccc6)n5)cc4)c3)ccc21 ; CC1(C)c2ccccc2-c2cc(-c3ccc(-c4ccc(-c5nc(-c6ccccc6)nc(-c6ccccc6)n5)cc4)cc3)ccc21]

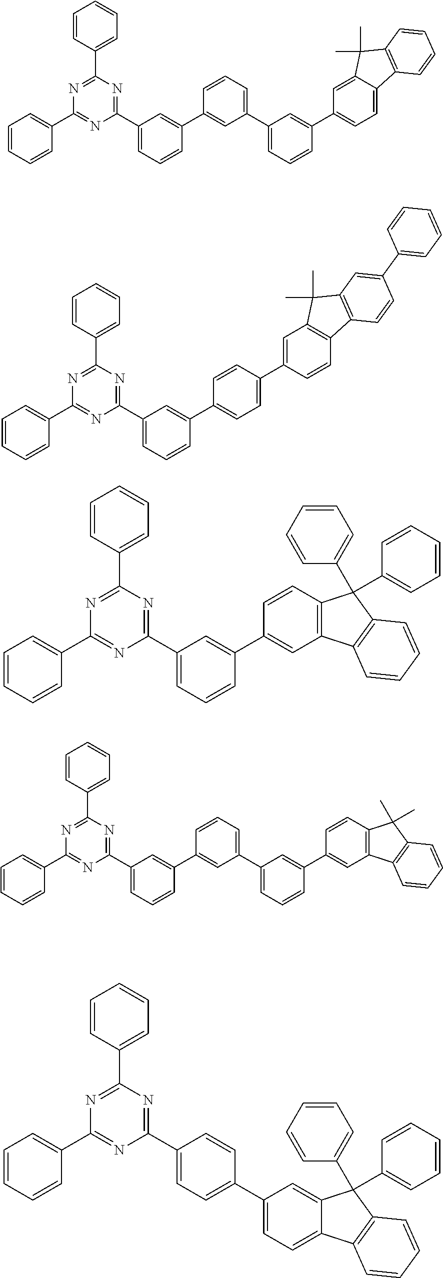 [SMILES: CC1(C)c2ccccc2-c2ccc(-c3cccc(-c4cccc(-c5cccc(-c6nc(-c7ccccc7)nc(-c7ccccc7)n6)c5)c4)c3)cc21 ; CC1(C)c2cc(-c3ccccc3)ccc2-c2ccc(-c3ccc(-c4cccc(-c5nc(-c6ccccc6)nc(-c6ccccc6)n5)c4)cc3)cc21 ; c1ccc(-c2nc(-c3ccccc3)nc(-c3cccc(-c4ccc5c(c4)-c4ccccc4C5(c4ccccc4)c4ccccc4)c3)n2)cc1 ; CC1(C)c2ccccc2-c2cc(-c3cccc(-c4cccc(-c5cccc(-c6nc(-c7ccccc7)nc(-c7ccccc7)n6)c5)c4)c3)ccc21 ; c1ccc(-c2nc(-c3ccccc3)nc(-c3ccc(-c4ccc5c(c4)C(c4ccccc4)(c4ccccc4)c4ccccc4-5)cc3)n2)cc1]

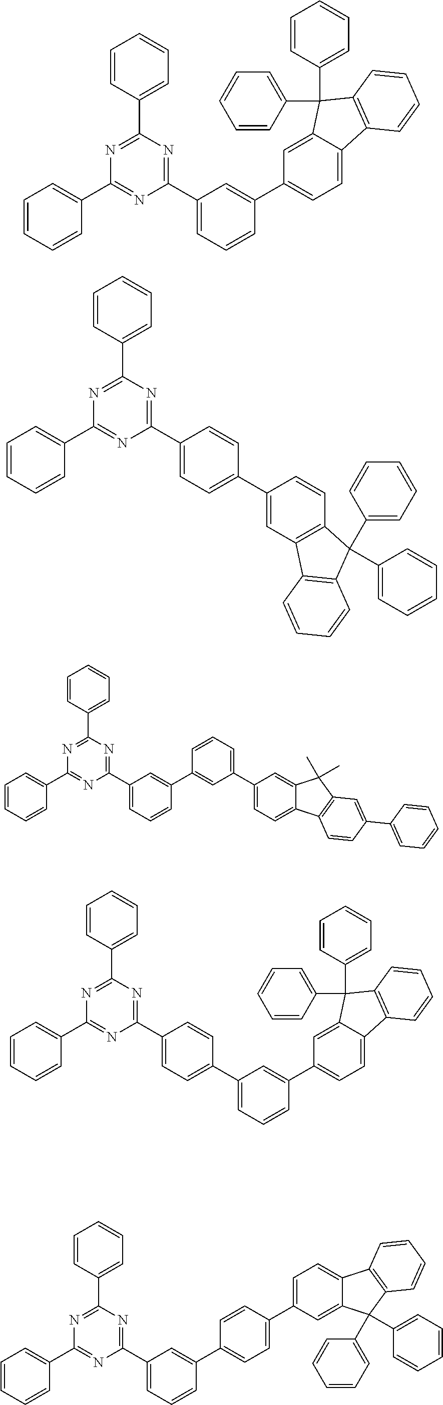

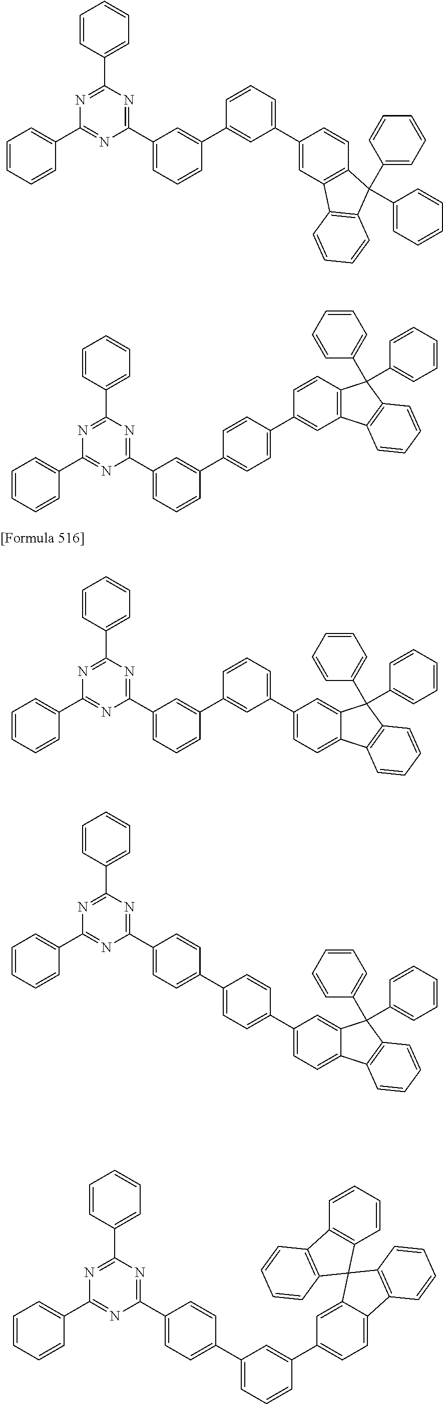 [SMILES: c1ccc(-c2nc(-c3ccccc3)nc(-c3cccc(-c4cccc(-c5ccc6c(c5)-c5ccccc5C6(c5ccccc5)c5ccccc5)c4)c3)n2)cc1 ; c1ccc(-c2nc(-c3ccccc3)nc(-c3cccc(-c4ccc(-c5ccc6c(c5)-c5ccccc5C6(c5ccccc5)c5ccccc5)cc4)c3)n2)cc1 ; c1ccc(-c2nc(-c3ccccc3)nc(-c3cccc(-c4cccc(-c5ccc6c(c5)C(c5ccccc5)(c5ccccc5)c5ccccc5-6)c4)c3)n2)cc1 ; c1ccc(-c2nc(-c3ccccc3)nc(-c3ccc(-c4ccc(-c5ccc6c(c5)C(c5ccccc5)(c5ccccc5)c5ccccc5-6)cc4)cc3)n2)cc1 ; c1ccc(-c2nc(-c3ccccc3)nc(-c3ccc(-c4cccc(-c5ccc6c(c5)C5(c7ccccc7-c7ccccc75)c5ccccc5-6)c4)cc3)n2)cc1]

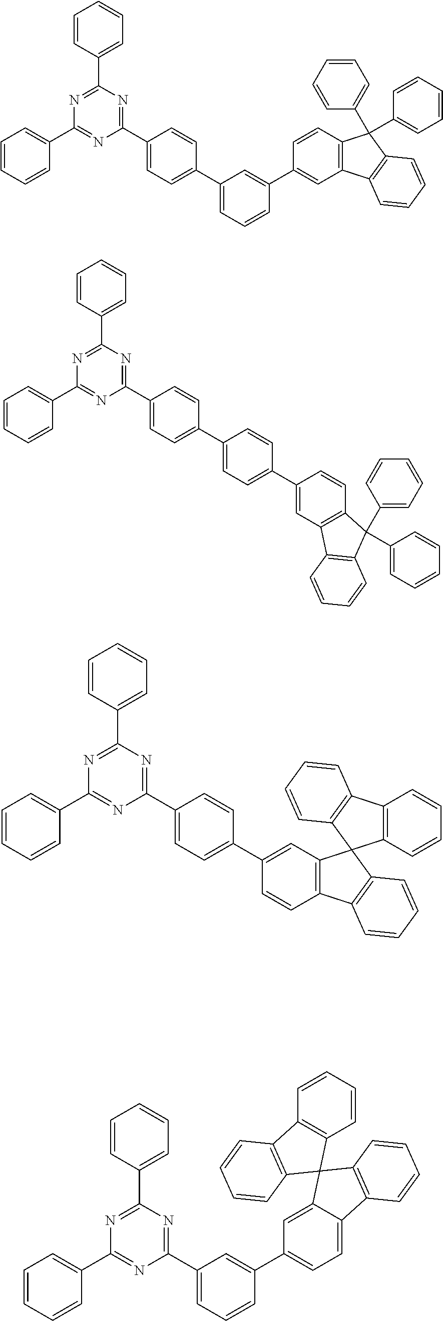 [SMILES: c1ccc(-c2nc(-c3ccccc3)nc(-c3ccc(-c4cccc(-c5ccc6c(c5)-c5ccccc5C6(c5ccccc5)c5ccccc5)c4)cc3)n2)cc1 ; c1ccc(-c2nc(-c3ccccc3)nc(-c3ccc(-c4ccc(-c5ccc6c(c5)-c5ccccc5C6(c5ccccc5)c5ccccc5)cc4)cc3)n2)cc1 ; c1ccc(-c2nc(-c3ccccc3)nc(-c3ccc(-c4ccc5c(c4)C4(c6ccccc6-c6ccccc64)c4ccccc4-5)cc3)n2)cc1 ; c1ccc(-c2nc(-c3ccccc3)nc(-c3cccc(-c4ccc5c(c4)C4(c6ccccc6-c6ccccc64)c4ccccc4-5)c3)n2)cc1]

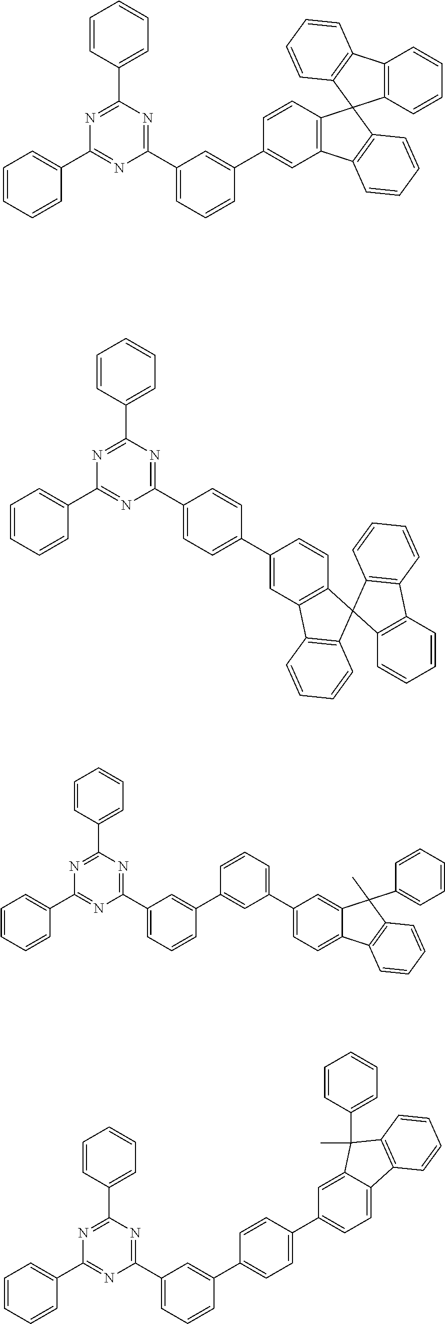 [SMILES: c1ccc(-c2nc(-c3ccccc3)nc(-c3cccc(-c4ccc5c(c4)-c4ccccc4C54c5ccccc5-c5ccccc54)c3)n2)cc1 ; c1ccc(-c2nc(-c3ccccc3)nc(-c3ccc(-c4ccc5c(c4)-c4ccccc4C54c5ccccc5-c5ccccc54)cc3)n2)cc1 ; CC1(c2ccccc2)c2ccccc2-c2ccc(-c3cccc(-c4cccc(-c5nc(-c6ccccc6)nc(-c6ccccc6)n5)c4)c3)cc21 ; CC1(c2ccccc2)c2ccccc2-c2ccc(-c3ccc(-c4cccc(-c5nc(-c6ccccc6)nc(-c6ccccc6)n5)c4)cc3)cc21]

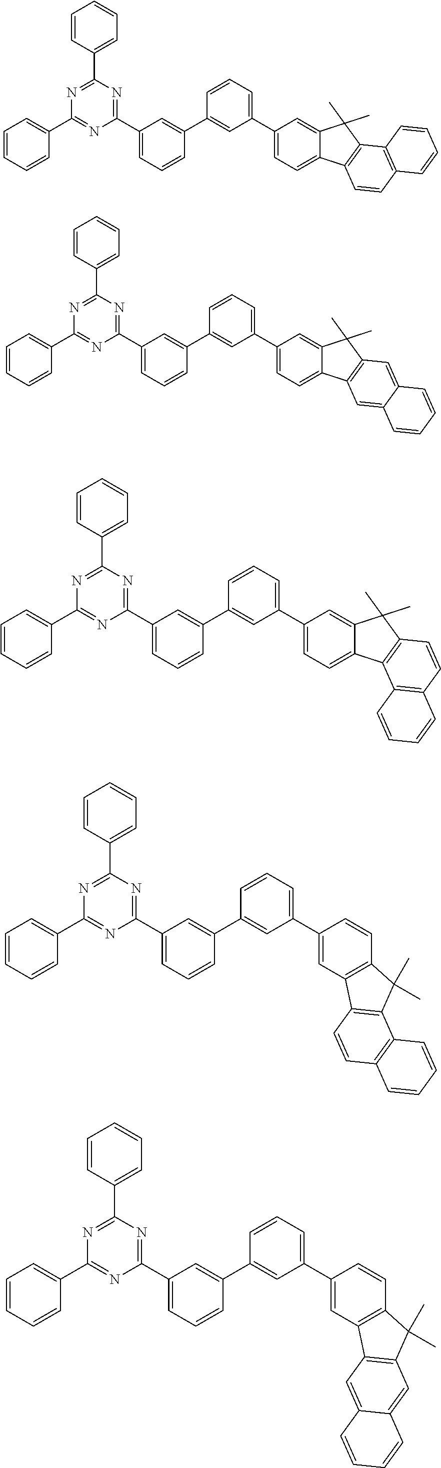 [SMILES: CC1(C)c2cc(-c3cccc(-c4cccc(-c5nc(-c6ccccc6)nc(-c6ccccc6)n5)c4)c3)ccc2-c2ccc3ccccc3c21 ; CC1(C)c2cc(-c3cccc(-c4cccc(-c5nc(-c6ccccc6)nc(-c6ccccc6)n5)c4)c3)ccc2-c2cc3ccccc3cc21 ; CC1(C)c2cc(-c3cccc(-c4cccc(-c5nc(-c6ccccc6)nc(-c6ccccc6)n5)c4)c3)ccc2-c2c1ccc1ccccc21 ; CC1(C)c2ccc(-c3cccc(-c4cccc(-c5nc(-c6ccccc6)nc(-c6ccccc6)n5)c4)c3)cc2-c2ccc3ccccc3c21 ; CC1(C)c2ccc(-c3cccc(-c4cccc(-c5nc(-c6ccccc6)nc(-c6ccccc6)n5)c4)c3)cc2-c2cc3ccccc3cc21]

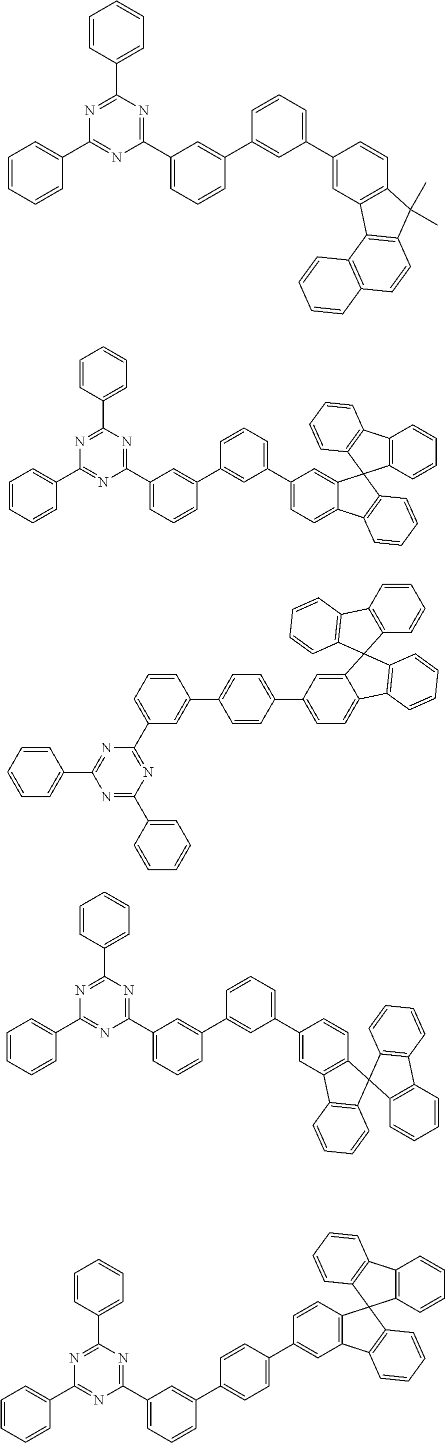 [SMILES: CC1(C)c2ccc(-c3cccc(-c4cccc(-c5nc(-c6ccccc6)nc(-c6ccccc6)n5)c4)c3)cc2-c2c1ccc1ccccc21 ; c1ccc(-c2nc(-c3ccccc3)nc(-c3cccc(-c4cccc(-c5ccc6c(c5)C5(c7ccccc7-c7ccccc75)c5ccccc5-6)c4)c3)n2)cc1 ; c1ccc(-c2nc(-c3ccccc3)nc(-c3cccc(-c4ccc(-c5ccc6c(c5)C5(c7ccccc7-c7ccccc75)c5ccccc5-6)cc4)c3)n2)cc1 ; c1ccc(-c2nc(-c3ccccc3)nc(-c3cccc(-c4cccc(-c5ccc6c(c5)-c5ccccc5C65c6ccccc6-c6ccccc65)c4)c3)n2)cc1 ; c1ccc(-c2nc(-c3ccccc3)nc(-c3cccc(-c4ccc(-c5ccc6c(c5)-c5ccccc5C65c6ccccc6-c6ccccc65)cc4)c3)n2)cc1]

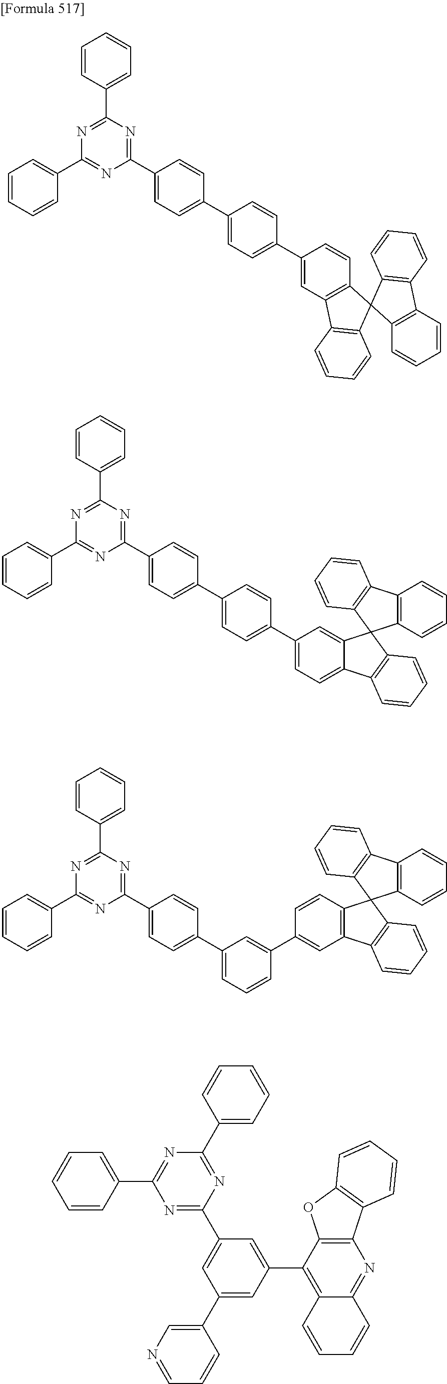 [SMILES: c1ccc(-c2nc(-c3ccccc3)nc(-c3ccc(-c4ccc(-c5ccc6c(c5)-c5ccccc5C65c6ccccc6-c6ccccc65)cc4)cc3)n2)cc1 ; c1ccc(-c2nc(-c3ccccc3)nc(-c3ccc(-c4ccc(-c5ccc6c(c5)C5(c7ccccc7-c7ccccc75)c5ccccc5-6)cc4)cc3)n2)cc1 ; c1ccc(-c2nc(-c3ccccc3)nc(-c3ccc(-c4cccc(-c5ccc6c(c5)-c5ccccc5C65c6ccccc6-c6ccccc65)c4)cc3)n2)cc1 ; c1ccc(-c2nc(-c3ccccc3)nc(-c3cc(-c4cccnc4)cc(-c4c5ccccc5nc5c4oc4ccccc45)c3)n2)cc1]

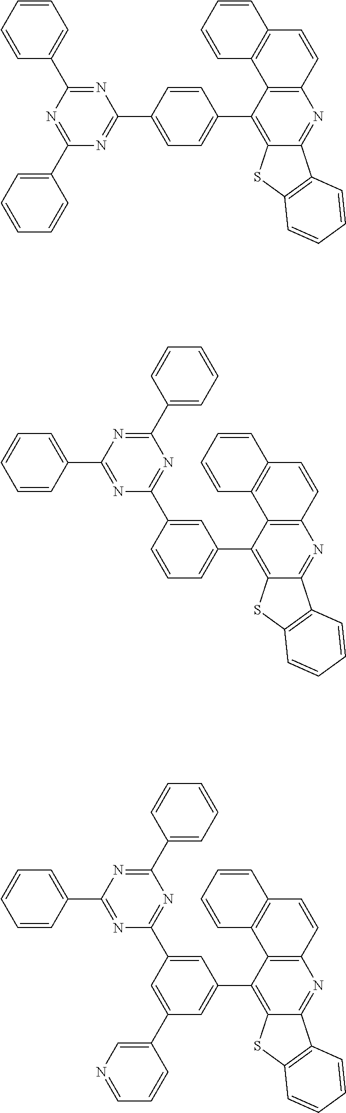 [SMILES: c1ccc(-c2nc(-c3ccccc3)nc(-c3ccc(-c4c5sc6ccccc6c5nc5ccc6ccccc6c45)cc3)n2)cc1 ; c1ccc(-c2nc(-c3ccccc3)nc(-c3cccc(-c4c5sc6ccccc6c5nc5ccc6ccccc6c45)c3)n2)cc1 ; c1ccc(-c2nc(-c3ccccc3)nc(-c3cc(-c4cccnc4)cc(-c4c5sc6ccccc6c5nc5ccc6ccccc6c45)c3)n2)cc1]

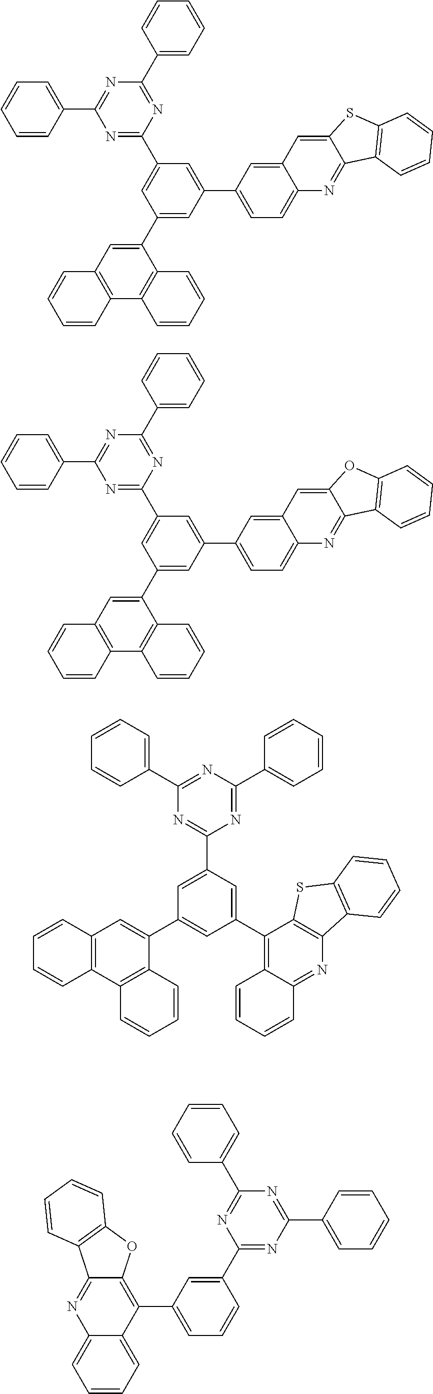

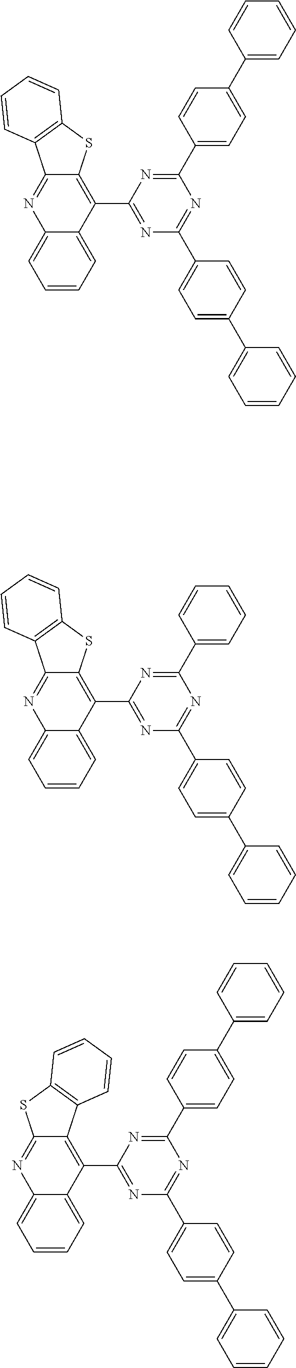 [SMILES: c1ccc(-c2ccc(-c3nc(-c4ccc(-c5ccccc5)cc4)nc(-c4c5ccccc5nc5c4sc4ccccc45)n3)cc2)cc1 ; c1ccc(-c2ccc(-c3nc(-c4ccccc4)nc(-c4c5ccccc5nc5c4sc4ccccc45)n3)cc2)cc1 ; c1ccc(-c2ccc(-c3nc(-c4ccc(-c5ccccc5)cc4)nc(-c4c5ccccc5nc5sc6ccccc6c45)n3)cc2)cc1]

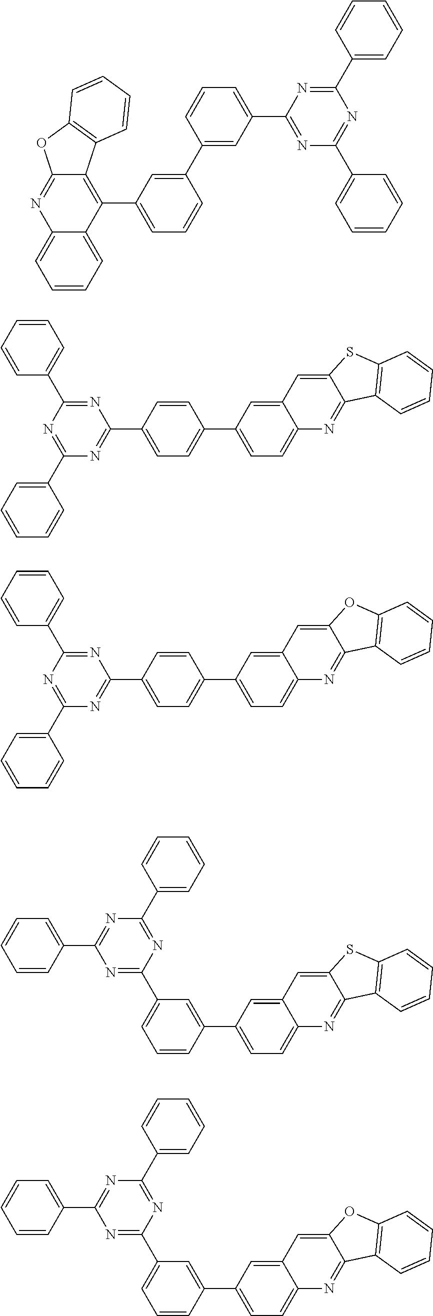

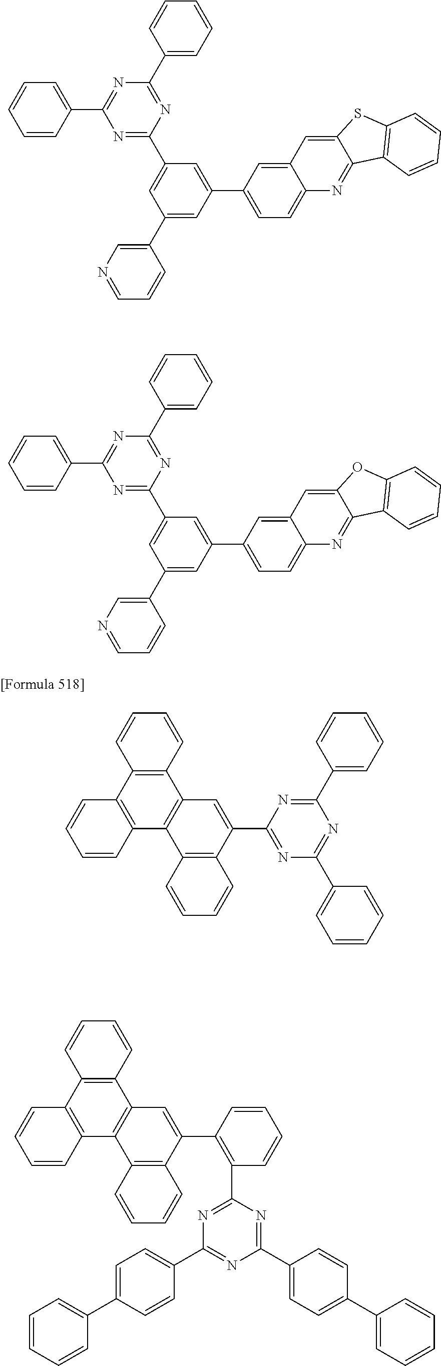 [SMILES: c1ccc(-c2nc(-c3ccccc3)nc(-c3cc(-c4cccnc4)cc(-c4ccc5nc6c(cc5c4)sc4ccccc46)c3)n2)cc1 ; c1ccc(-c2nc(-c3ccccc3)nc(-c3cc(-c4cccnc4)cc(-c4ccc5nc6c(cc5c4)oc4ccccc46)c3)n2)cc1 ; c1ccc(-c2nc(-c3ccccc3)nc(-c3cc4c5ccccc5c5ccccc5c4c4ccccc34)n2)cc1 ; c1ccc(-c2ccc(-c3nc(-c4ccc(-c5ccccc5)cc4)nc(-c4ccccc4-c4cc5c6ccccc6c6ccccc6c5c5ccccc45)n3)cc2)cc1]

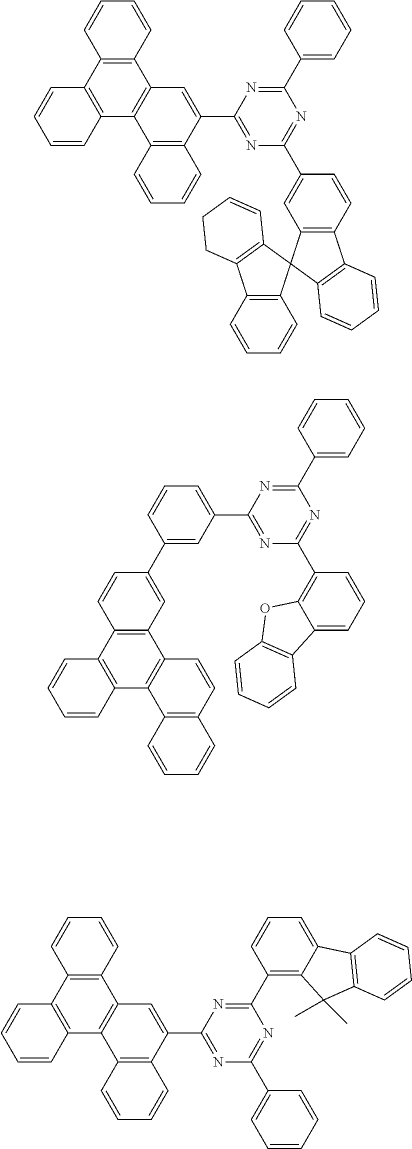 [SMILES: C1=CC2=C(CC1)c1ccccc1C21c2ccccc2-c2ccc(-c3nc(-c4ccccc4)nc(-c4cc5c6ccccc6c6ccccc6c5c5ccccc45)n3)cc21 ; c1ccc(-c2nc(-c3cccc(-c4ccc5c6ccccc6c6c7ccccc7ccc6c5c4)c3)nc(-c3cccc4c3oc3ccccc34)n2)cc1 ; CC1(C)c2ccccc2-c2cccc(-c3nc(-c4ccccc4)nc(-c4cc5c6ccccc6c6ccccc6c5c5ccccc45)n3)c21]

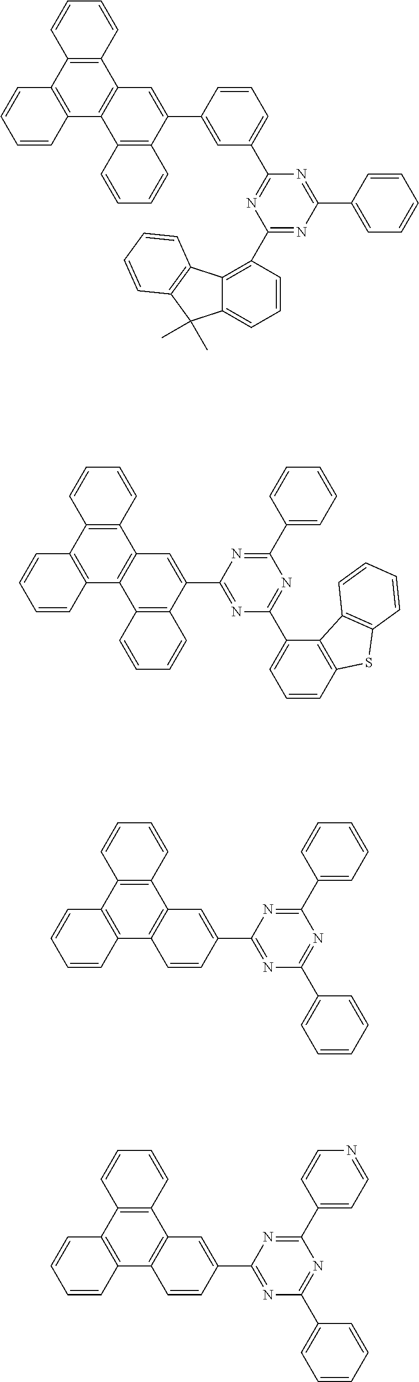 [SMILES: CC1(C)c2ccccc2-c2c(-c3nc(-c4ccccc4)nc(-c4cccc(-c5cc6c7ccccc7c7ccccc7c6c6ccccc56)c4)n3)cccc21 ; c1ccc(-c2nc(-c3cc4c5ccccc5c5ccccc5c4c4ccccc34)nc(-c3cccc4sc5ccccc5c34)n2)cc1 ; c1ccc(-c2nc(-c3ccccc3)nc(-c3ccc4c5ccccc5c5ccccc5c4c3)n2)cc1 ; c1ccc(-c2nc(-c3ccncc3)nc(-c3ccc4c5ccccc5c5ccccc5c4c3)n2)cc1]

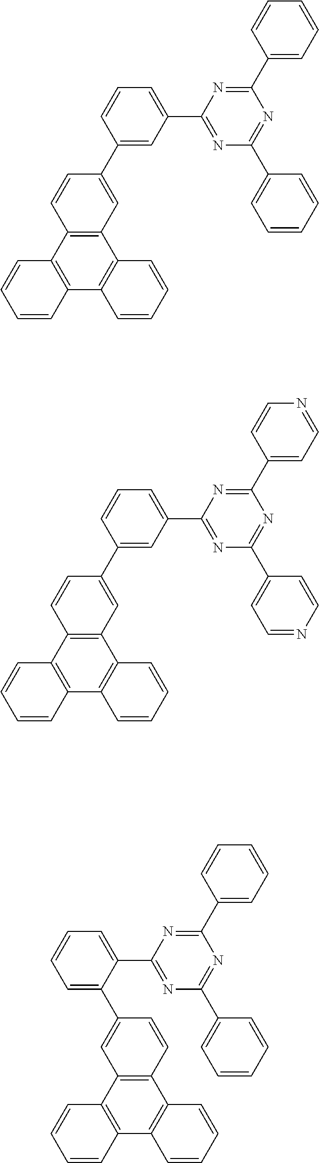 [SMILES: c1ccc(-c2nc(-c3ccccc3)nc(-c3cccc(-c4ccc5c6ccccc6c6ccccc6c5c4)c3)n2)cc1 ; c1cc(-c2ccc3c4ccccc4c4ccccc4c3c2)cc(-c2nc(-c3ccncc3)nc(-c3ccncc3)n2)c1 ; c1ccc(-c2nc(-c3ccccc3)nc(-c3ccccc3-c3ccc4c5ccccc5c5ccccc5c4c3)n2)cc1]

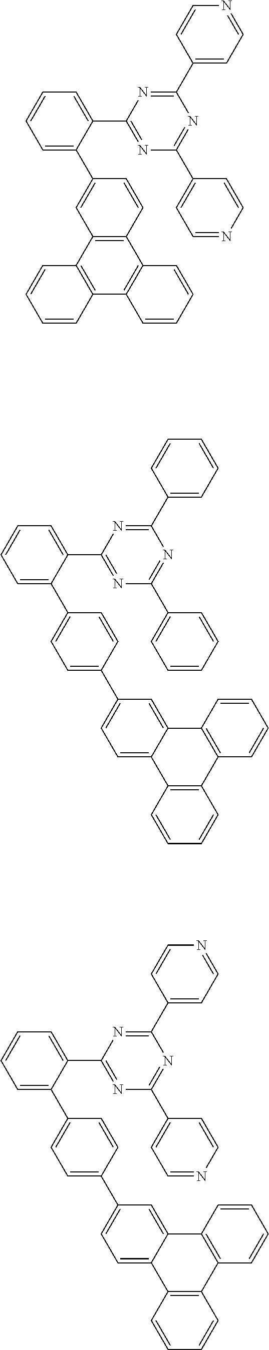 [SMILES: c1ccc(-c2nc(-c3ccncc3)nc(-c3ccncc3)n2)c(-c2ccc3c4ccccc4c4ccccc4c3c2)c1 ; c1ccc(-c2nc(-c3ccccc3)nc(-c3ccccc3-c3ccc(-c4ccc5c6ccccc6c6ccccc6c5c4)cc3)n2)cc1 ; c1ccc(-c2nc(-c3ccncc3)nc(-c3ccncc3)n2)c(-c2ccc(-c3ccc4c5ccccc5c5ccccc5c4c3)cc2)c1]

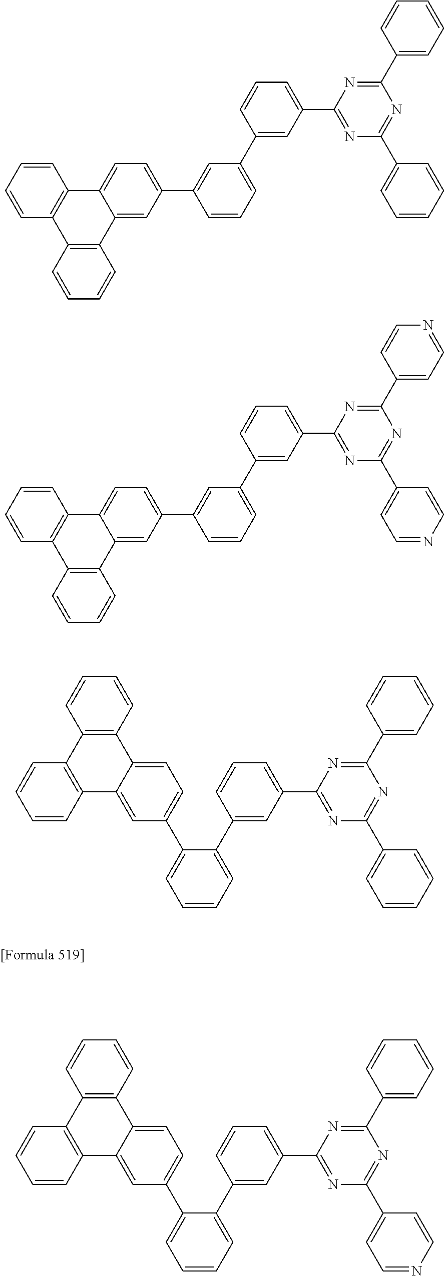 [SMILES: c1ccc(-c2nc(-c3ccccc3)nc(-c3cccc(-c4cccc(-c5ccc6c7ccccc7c7ccccc7c6c5)c4)c3)n2)cc1 ; c1cc(-c2cccc(-c3nc(-c4ccncc4)nc(-c4ccncc4)n3)c2)cc(-c2ccc3c4ccccc4c4ccccc4c3c2)c1 ; c1ccc(-c2nc(-c3ccccc3)nc(-c3cccc(-c4ccccc4-c4ccc5c6ccccc6c6ccccc6c5c4)c3)n2)cc1 ; c1ccc(-c2nc(-c3ccncc3)nc(-c3cccc(-c4ccccc4-c4ccc5c6ccccc6c6ccccc6c5c4)c3)n2)cc1]

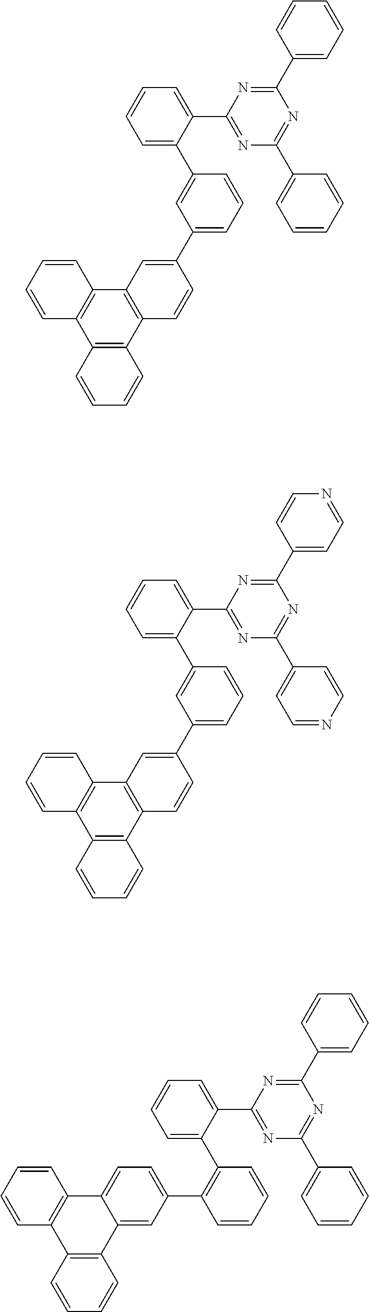 [SMILES: c1ccc(-c2nc(-c3ccccc3)nc(-c3ccccc3-c3cccc(-c4ccc5c6ccccc6c6ccccc6c5c4)c3)n2)cc1 ; c1cc(-c2ccc3c4ccccc4c4ccccc4c3c2)cc(-c2ccccc2-c2nc(-c3ccncc3)nc(-c3ccncc3)n2)c1 ; c1ccc(-c2nc(-c3ccccc3)nc(-c3ccccc3-c3ccccc3-c3ccc4c5ccccc5c5ccccc5c4c3)n2)cc1]

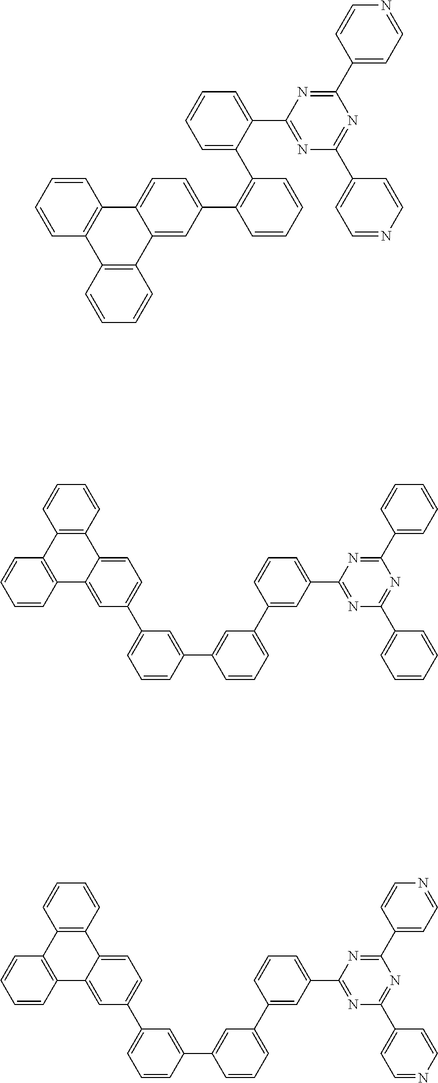 [SMILES: c1ccc(-c2ccccc2-c2nc(-c3ccncc3)nc(-c3ccncc3)n2)c(-c2ccc3c4ccccc4c4ccccc4c3c2)c1 ; c1ccc(-c2nc(-c3ccccc3)nc(-c3cccc(-c4cccc(-c5cccc(-c6ccc7c8ccccc8c8ccccc8c7c6)c5)c4)c3)n2)cc1 ; c1cc(-c2cccc(-c3ccc4c5ccccc5c5ccccc5c4c3)c2)cc(-c2cccc(-c3nc(-c4ccncc4)nc(-c4ccncc4)n3)c2)c1]

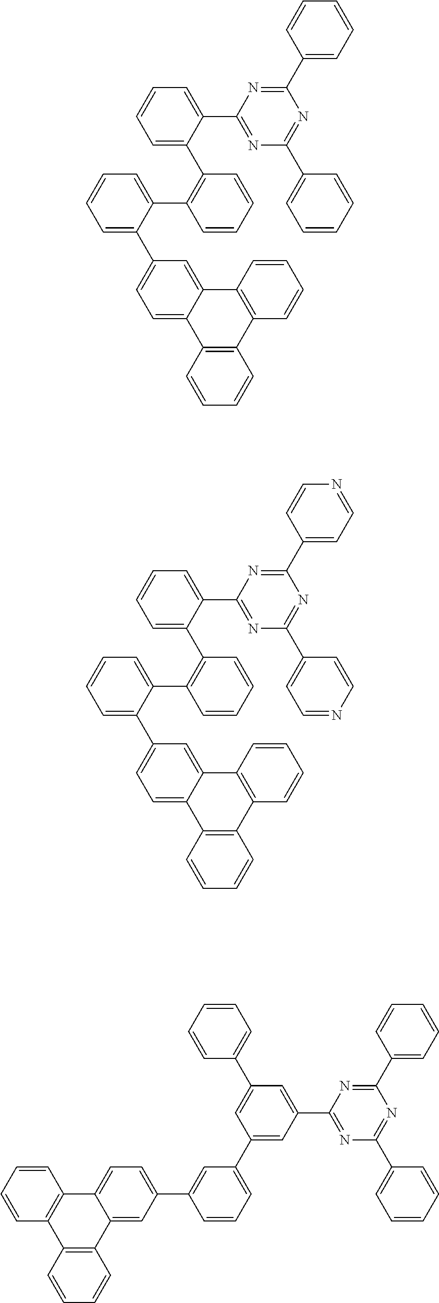 [SMILES: c1ccc(-c2nc(-c3ccccc3)nc(-c3ccccc3-c3ccccc3-c3ccccc3-c3ccc4c5ccccc5c5ccccc5c4c3)n2)cc1 ; c1ccc(-c2ccccc2-c2ccccc2-c2nc(-c3ccncc3)nc(-c3ccncc3)n2)c(-c2ccc3c4ccccc4c4ccccc4c3c2)c1 ; c1ccc(-c2cc(-c3cccc(-c4ccc5c6ccccc6c6ccccc6c5c4)c3)cc(-c3nc(-c4ccccc4)nc(-c4ccccc4)n3)c2)cc1]

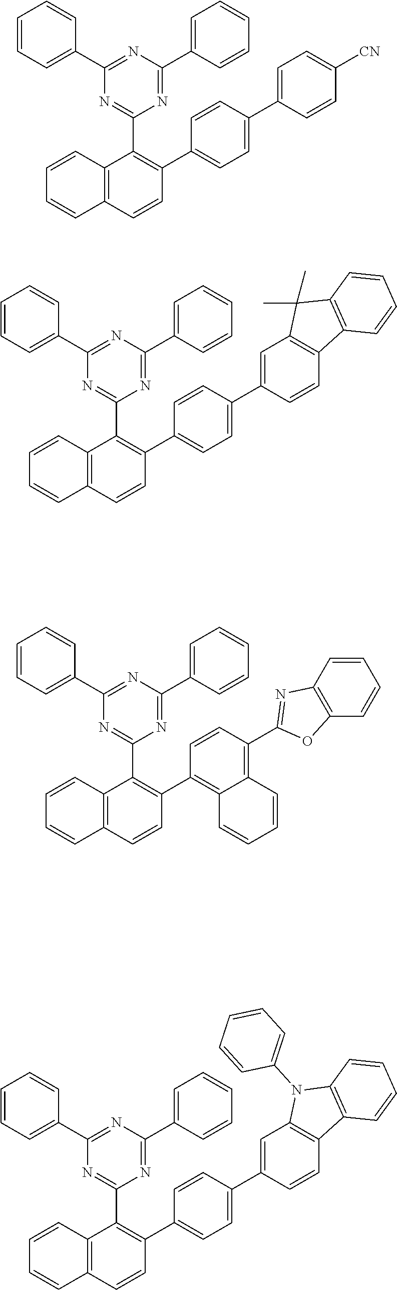 [SMILES: N#Cc1ccc(-c2ccc(-c3ccc4ccccc4c3-c3nc(-c4ccccc4)nc(-c4ccccc4)n3)cc2)cc1 ; CC1(C)c2ccccc2-c2ccc(-c3ccc(-c4ccc5ccccc5c4-c4nc(-c5ccccc5)nc(-c5ccccc5)n4)cc3)cc21 ; c1ccc(-c2nc(-c3ccccc3)nc(-c3c(-c4ccc(-c5nc6ccccc6o5)c5ccccc45)ccc4ccccc34)n2)cc1 ; c1ccc(-c2nc(-c3ccccc3)nc(-c3c(-c4ccc(-c5ccc6c7ccccc7n(-c7ccccc7)c6c5)cc4)ccc4ccccc34)n2)cc1]

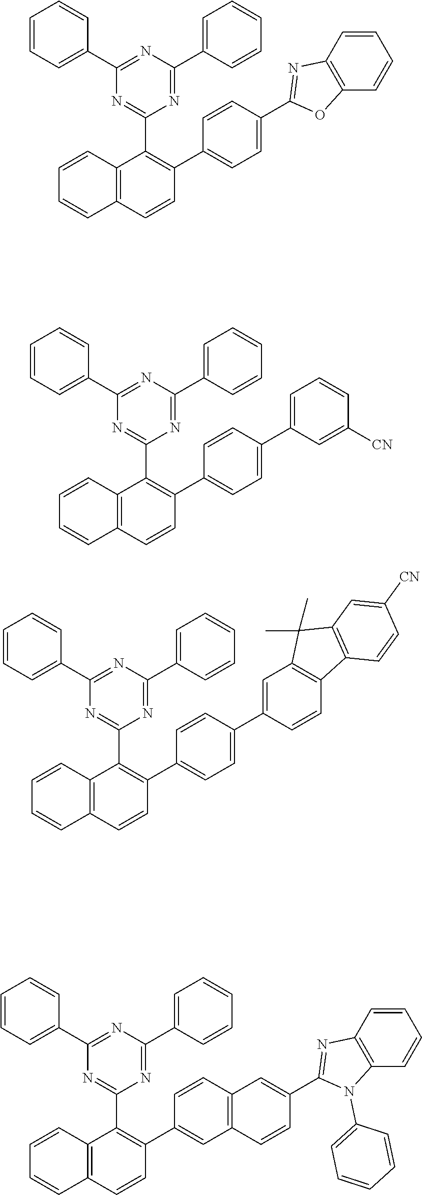 [SMILES: c1ccc(-c2nc(-c3ccccc3)nc(-c3c(-c4ccc(-c5nc6ccccc6o5)cc4)ccc4ccccc34)n2)cc1 ; N#Cc1cccc(-c2ccc(-c3ccc4ccccc4c3-c3nc(-c4ccccc4)nc(-c4ccccc4)n3)cc2)c1 ; CC1(C)c2cc(C#N)ccc2-c2ccc(-c3ccc(-c4ccc5ccccc5c4-c4nc(-c5ccccc5)nc(-c5ccccc5)n4)cc3)cc21 ; c1ccc(-c2nc(-c3ccccc3)nc(-c3c(-c4ccc5cc(-c6nc7ccccc7n6-c6ccccc6)ccc5c4)ccc4ccccc34)n2)cc1]

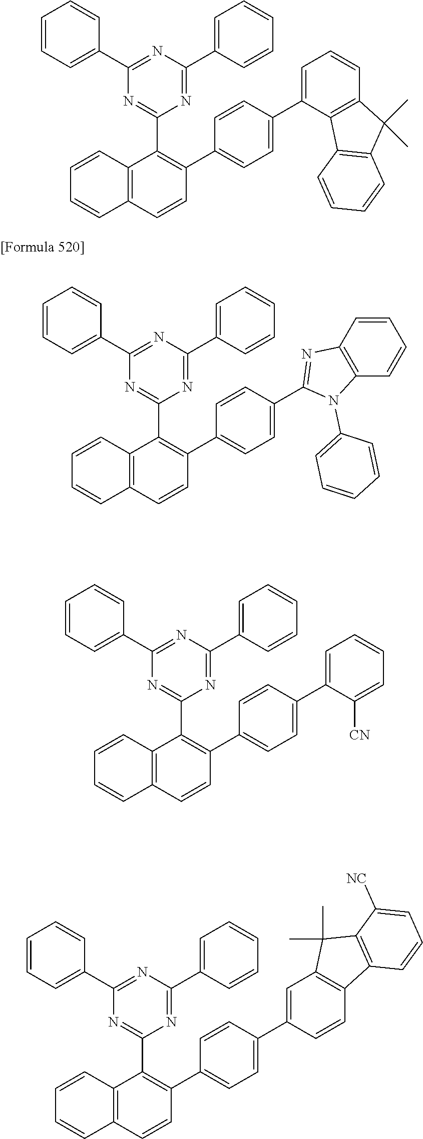 [SMILES: CC1(C)c2ccccc2-c2c(-c3ccc(-c4ccc5ccccc5c4-c4nc(-c5ccccc5)nc(-c5ccccc5)n4)cc3)cccc21 ; c1ccc(-c2nc(-c3ccccc3)nc(-c3c(-c4ccc(-c5nc6ccccc6n5-c5ccccc5)cc4)ccc4ccccc34)n2)cc1 ; N#Cc1ccccc1-c1ccc(-c2ccc3ccccc3c2-c2nc(-c3ccccc3)nc(-c3ccccc3)n2)cc1 ; CC1(C)c2cc(-c3ccc(-c4ccc5ccccc5c4-c4nc(-c5ccccc5)nc(-c5ccccc5)n4)cc3)ccc2-c2cccc(C#N)c21]

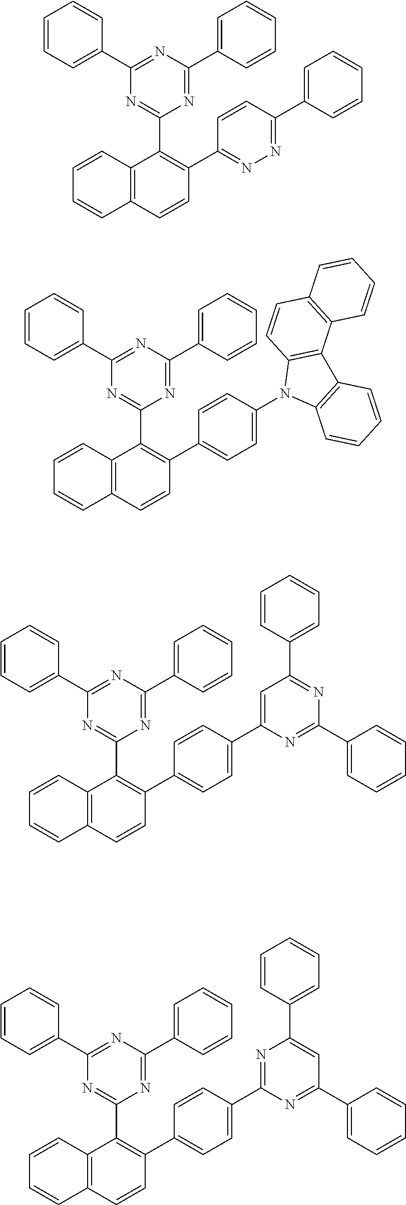 [SMILES: c1ccc(-c2ccc(-c3ccc4ccccc4c3-c3nc(-c4ccccc4)nc(-c4ccccc4)n3)nn2)cc1 ; c1ccc(-c2nc(-c3ccccc3)nc(-c3c(-c4ccc(-n5c6ccccc6c6c7ccccc7ccc65)cc4)ccc4ccccc34)n2)cc1 ; c1ccc(-c2cc(-c3ccc(-c4ccc5ccccc5c4-c4nc(-c5ccccc5)nc(-c5ccccc5)n4)cc3)nc(-c3ccccc3)n2)cc1 ; c1ccc(-c2cc(-c3ccccc3)nc(-c3ccc(-c4ccc5ccccc5c4-c4nc(-c5ccccc5)nc(-c5ccccc5)n4)cc3)n2)cc1]

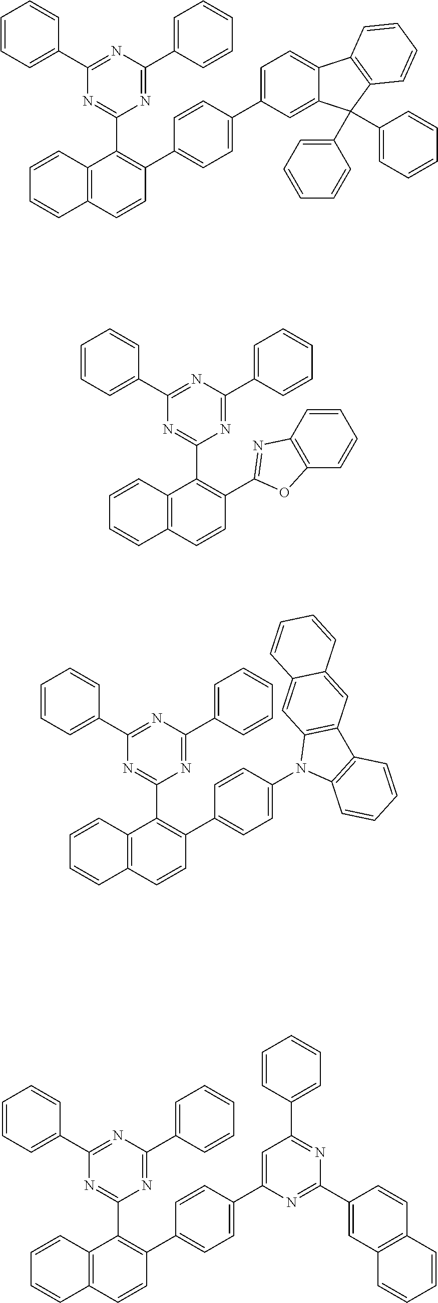 [SMILES: c1ccc(-c2nc(-c3ccccc3)nc(-c3c(-c4ccc(-c5ccc6c(c5)C(c5ccccc5)(c5ccccc5)c5ccccc5-6)cc4)ccc4ccccc34)n2)cc1 ; c1ccc(-c2nc(-c3ccccc3)nc(-c3c(-c4nc5ccccc5o4)ccc4ccccc34)n2)cc1 ; c1ccc(-c2nc(-c3ccccc3)nc(-c3c(-c4ccc(-n5c6ccccc6c6cc7ccccc7cc65)cc4)ccc4ccccc34)n2)cc1 ; c1ccc(-c2cc(-c3ccc(-c4ccc5ccccc5c4-c4nc(-c5ccccc5)nc(-c5ccccc5)n4)cc3)nc(-c3ccc4ccccc4c3)n2)cc1]

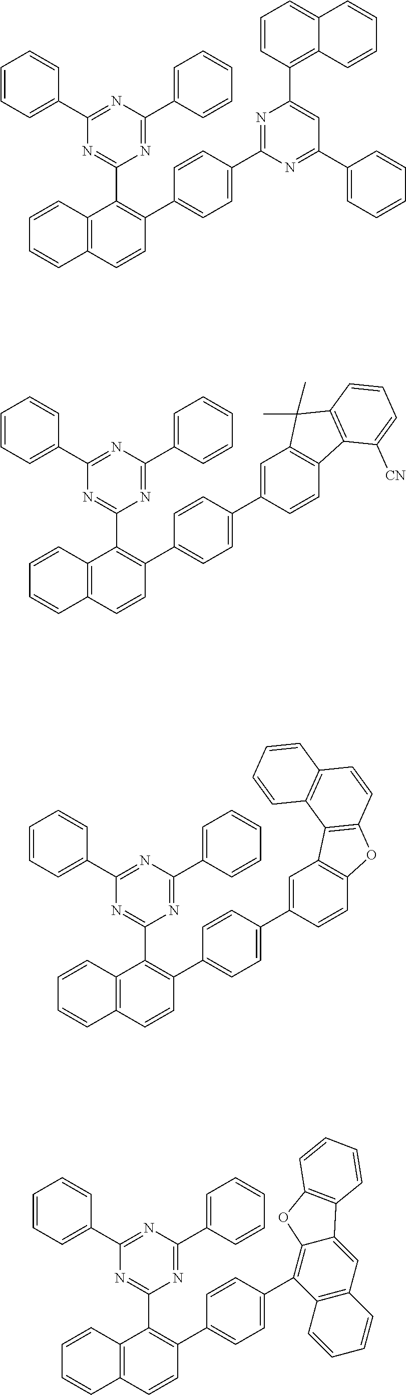 [SMILES: c1ccc(-c2cc(-c3cccc4ccccc34)nc(-c3ccc(-c4ccc5ccccc5c4-c4nc(-c5ccccc5)nc(-c5ccccc5)n4)cc3)n2)cc1 ; CC1(C)c2cc(-c3ccc(-c4ccc5ccccc5c4-c4nc(-c5ccccc5)nc(-c5ccccc5)n4)cc3)ccc2-c2c(C#N)cccc21 ; c1ccc(-c2nc(-c3ccccc3)nc(-c3c(-c4ccc(-c5ccc6oc7ccc8ccccc8c7c6c5)cc4)ccc4ccccc34)n2)cc1 ; c1ccc(-c2nc(-c3ccccc3)nc(-c3c(-c4ccc(-c5c6ccccc6cc6c5oc5ccccc56)cc4)ccc4ccccc34)n2)cc1]

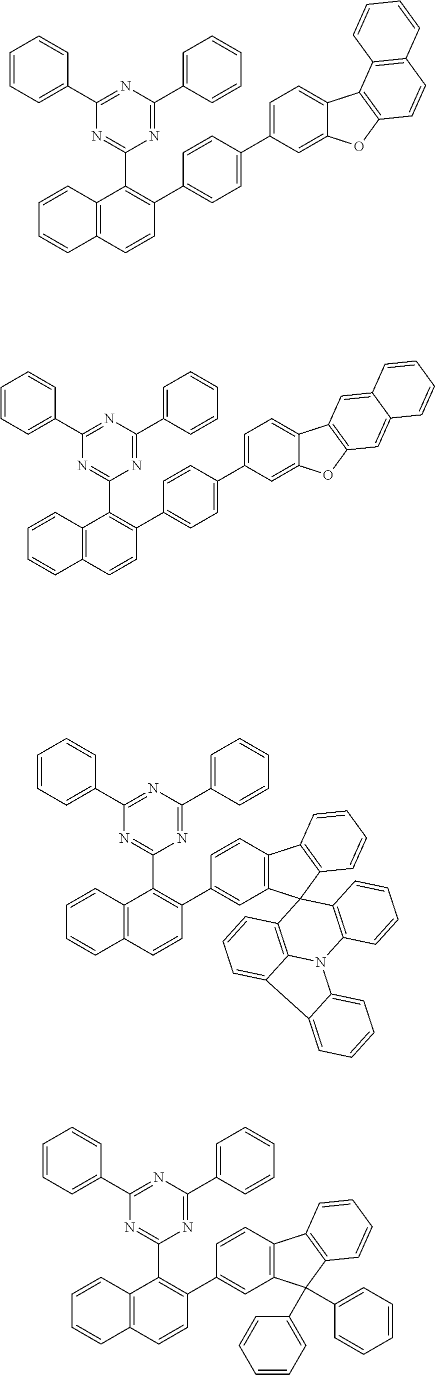 [SMILES: c1ccc(-c2nc(-c3ccccc3)nc(-c3c(-c4ccc(-c5ccc6c(c5)oc5ccc7ccccc7c56)cc4)ccc4ccccc34)n2)cc1 ; c1ccc(-c2nc(-c3ccccc3)nc(-c3c(-c4ccc(-c5ccc6c(c5)oc5cc7ccccc7cc56)cc4)ccc4ccccc34)n2)cc1 ; c1ccc(-c2nc(-c3ccccc3)nc(-c3c(-c4ccc5c(c4)C4(c6ccccc6-5)c5ccccc5-n5c6ccccc6c6cccc4c65)ccc4ccccc34)n2)cc1 ; c1ccc(-c2nc(-c3ccccc3)nc(-c3c(-c4ccc5c(c4)C(c4ccccc4)(c4ccccc4)c4ccccc4-5)ccc4ccccc34)n2)cc1]

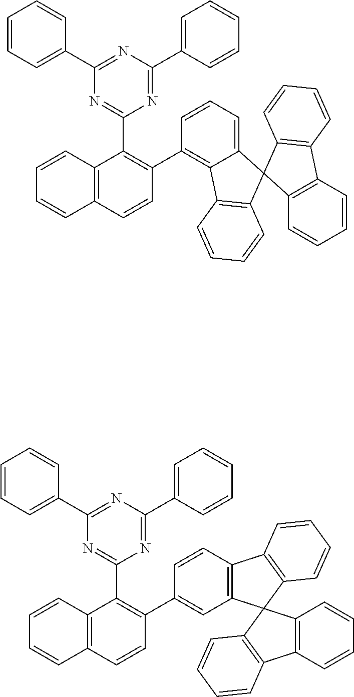 [SMILES: c1ccc(-c2nc(-c3ccccc3)nc(-c3c(-c4cccc5c4-c4ccccc4C54c5ccccc5-c5ccccc54)ccc4ccccc34)n2)cc1 ; c1ccc(-c2nc(-c3ccccc3)nc(-c3c(-c4ccc5c(c4)C4(c6ccccc6-c6ccccc64)c4ccccc4-5)ccc4ccccc34)n2)cc1]

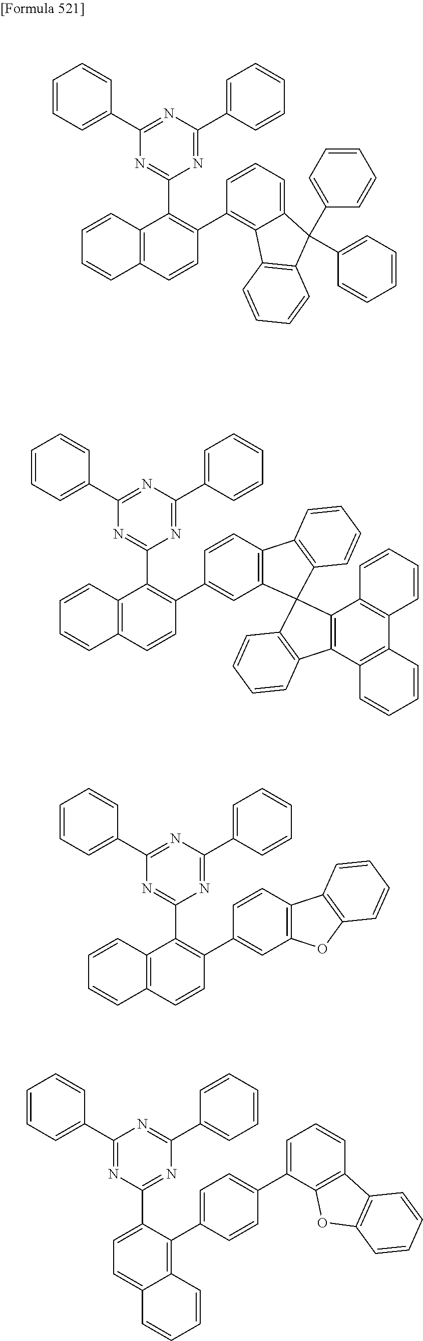 [SMILES: c1ccc(-c2nc(-c3ccccc3)nc(-c3c(-c4cccc5c4-c4ccccc4C5(c4ccccc4)c4ccccc4)ccc4ccccc34)n2)cc1 ; c1ccc(-c2nc(-c3ccccc3)nc(-c3c(-c4ccc5c(c4)C4(c6ccccc6-5)c5ccccc5-c5c4c4ccccc4c4ccccc54)ccc4ccccc34)n2)cc1 ; c1ccc(-c2nc(-c3ccccc3)nc(-c3c(-c4ccc5c(c4)oc4ccccc45)ccc4ccccc34)n2)cc1 ; c1ccc(-c2nc(-c3ccccc3)nc(-c3ccc4ccccc4c3-c3ccc(-c4cccc5c4oc4ccccc45)cc3)n2)cc1]

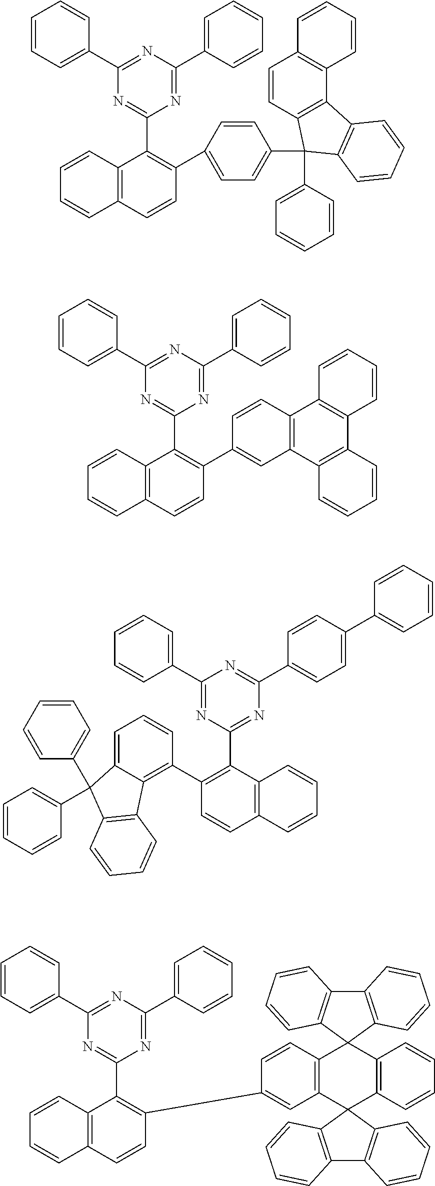 [SMILES: c1ccc(-c2nc(-c3ccccc3)nc(-c3c(-c4ccc(C5(c6ccccc6)c6ccccc6-c6c5ccc5ccccc65)cc4)ccc4ccccc34)n2)cc1 ; c1ccc(-c2nc(-c3ccccc3)nc(-c3c(-c4ccc5c6ccccc6c6ccccc6c5c4)ccc4ccccc34)n2)cc1 ; c1ccc(-c2ccc(-c3nc(-c4ccccc4)nc(-c4c(-c5cccc6c5-c5ccccc5C6(c5ccccc5)c5ccccc5)ccc5ccccc45)n3)cc2)cc1 ; c1ccc(-c2nc(-c3ccccc3)nc(-c3c(-c4ccc5c(c4)C4(c6ccccc6-c6ccccc64)c4ccccc4C54c5ccccc5-c5ccccc54)ccc4ccccc34)n2)cc1]

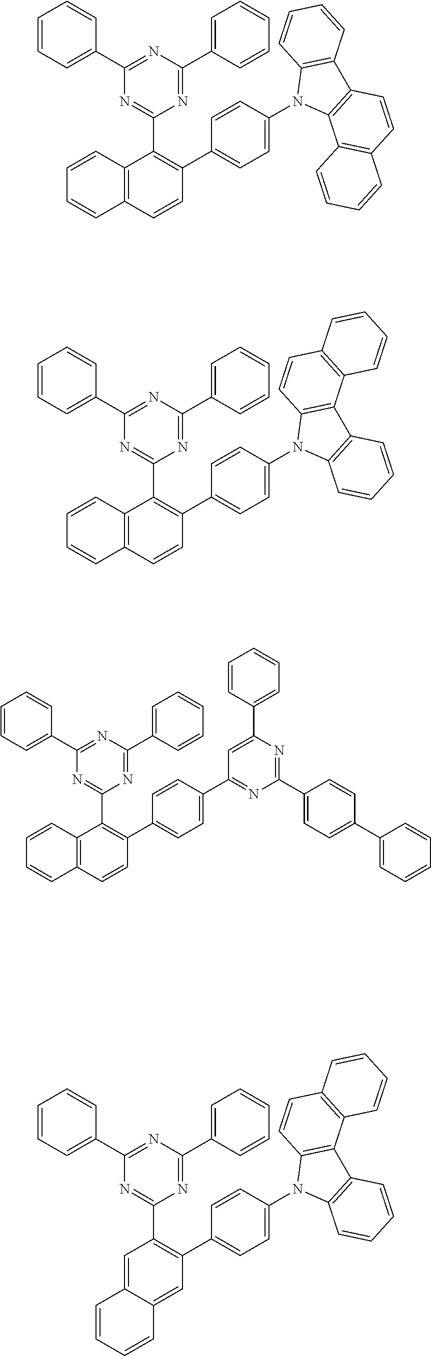 [SMILES: c1ccc(-c2nc(-c3ccccc3)nc(-c3c(-c4ccc(-n5c6ccccc6c6ccc7ccccc7c65)cc4)ccc4ccccc34)n2)cc1 ; c1ccc(-c2nc(-c3ccccc3)nc(-c3c(-c4ccc(-n5c6ccccc6c6c7ccccc7ccc65)cc4)ccc4ccccc34)n2)cc1 ; c1ccc(-c2ccc(-c3nc(-c4ccccc4)cc(-c4ccc(-c5ccc6ccccc6c5-c5nc(-c6ccccc6)nc(-c6ccccc6)n5)cc4)n3)cc2)cc1 ; c1ccc(-c2nc(-c3ccccc3)nc(-c3cc4ccccc4cc3-c3ccc(-n4c5ccccc5c5c6ccccc6ccc54)cc3)n2)cc1]

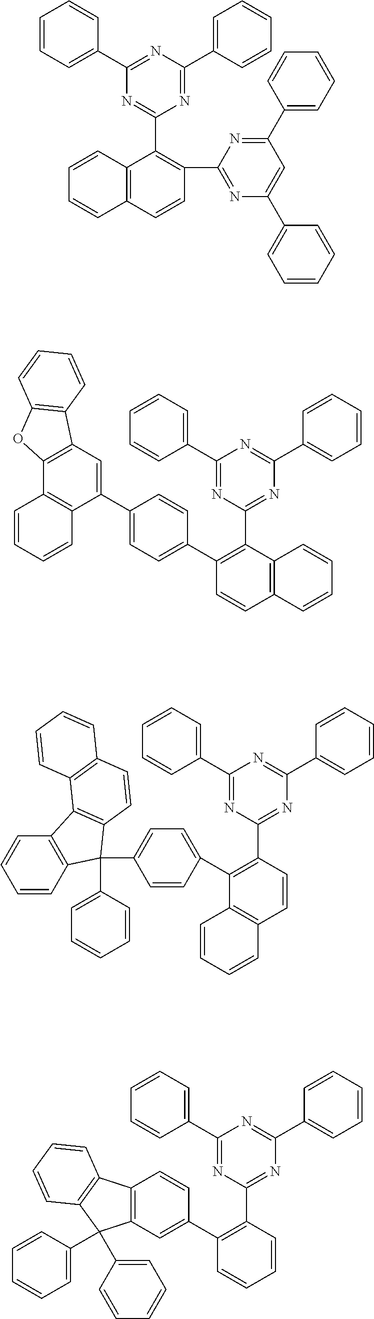 [SMILES: c1ccc(-c2cc(-c3ccccc3)nc(-c3ccc4ccccc4c3-c3nc(-c4ccccc4)nc(-c4ccccc4)n3)n2)cc1 ; c1ccc(-c2nc(-c3ccccc3)nc(-c3c(-c4ccc(-c5cc6c7ccccc7oc6c6ccccc56)cc4)ccc4ccccc34)n2)cc1 ; c1ccc(-c2nc(-c3ccccc3)nc(-c3ccc4ccccc4c3-c3ccc(C4(c5ccccc5)c5ccccc5-c5c4ccc4ccccc54)cc3)n2)cc1 ; c1ccc(-c2nc(-c3ccccc3)nc(-c3ccccc3-c3ccc4c(c3)C(c3ccccc3)(c3ccccc3)c3ccccc3-4)n2)cc1]

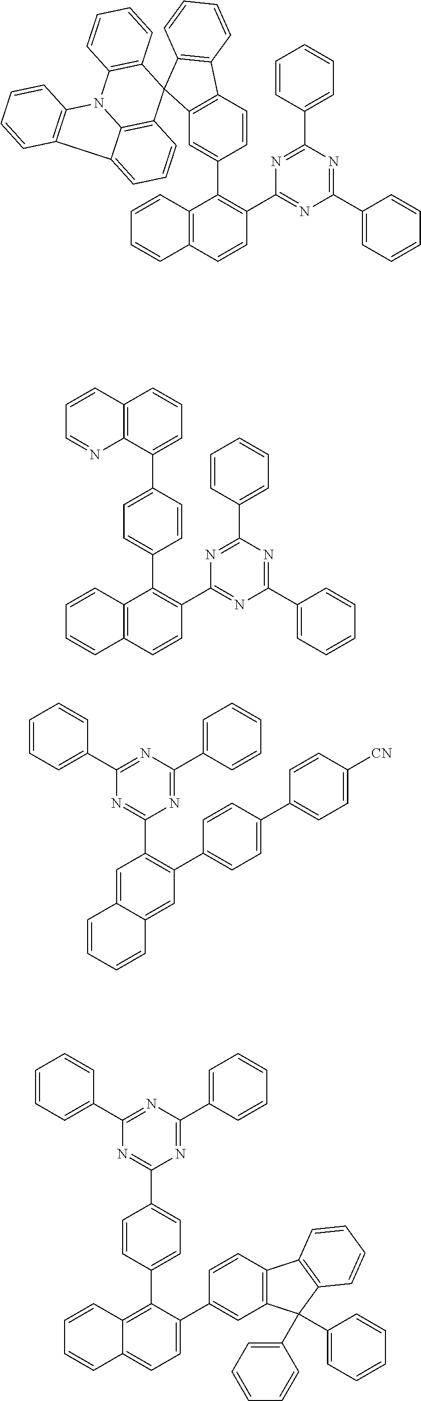 [SMILES: c1ccc(-c2nc(-c3ccccc3)nc(-c3ccc4ccccc4c3-c3ccc4c(c3)C3(c5ccccc5-4)c4ccccc4-n4c5ccccc5c5cccc3c54)n2)cc1 ; c1ccc(-c2nc(-c3ccccc3)nc(-c3ccc4ccccc4c3-c3ccc(-c4cccc5cccnc45)cc3)n2)cc1 ; N#Cc1ccc(-c2ccc(-c3cc4ccccc4cc3-c3nc(-c4ccccc4)nc(-c4ccccc4)n3)cc2)cc1 ; c1ccc(-c2nc(-c3ccccc3)nc(-c3ccc(-c4c(-c5ccc6c(c5)C(c5ccccc5)(c5ccccc5)c5ccccc5-6)ccc5ccccc45)cc3)n2)cc1]

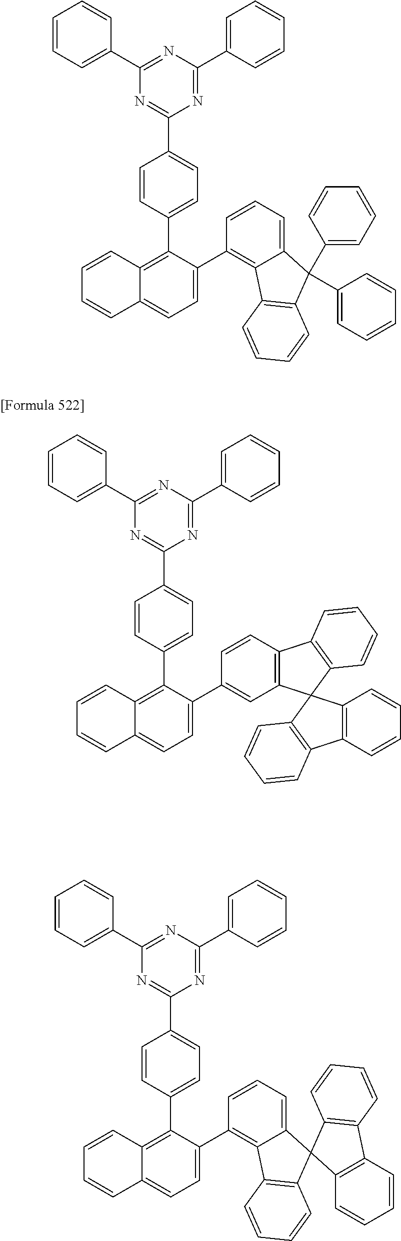 [SMILES: c1ccc(-c2nc(-c3ccccc3)nc(-c3ccc(-c4c(-c5cccc6c5-c5ccccc5C6(c5ccccc5)c5ccccc5)ccc5ccccc45)cc3)n2)cc1 ; c1ccc(-c2nc(-c3ccccc3)nc(-c3ccc(-c4c(-c5ccc6c(c5)C5(c7ccccc7-c7ccccc75)c5ccccc5-6)ccc5ccccc45)cc3)n2)cc1 ; c1ccc(-c2nc(-c3ccccc3)nc(-c3ccc(-c4c(-c5cccc6c5-c5ccccc5C65c6ccccc6-c6ccccc65)ccc5ccccc45)cc3)n2)cc1]

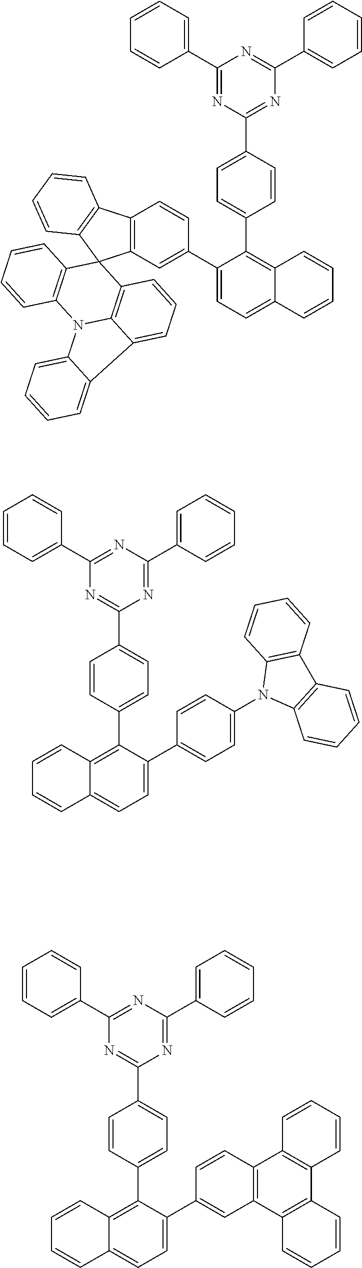 [SMILES: c1ccc(-c2nc(-c3ccccc3)nc(-c3ccc(-c4c(-c5ccc6c(c5)C5(c7ccccc7-6)c6ccccc6-n6c7ccccc7c7cccc5c76)ccc5ccccc45)cc3)n2)cc1 ; c1ccc(-c2nc(-c3ccccc3)nc(-c3ccc(-c4c(-c5ccc(-n6c7ccccc7c7ccccc76)cc5)ccc5ccccc45)cc3)n2)cc1 ; c1ccc(-c2nc(-c3ccccc3)nc(-c3ccc(-c4c(-c5ccc6c7ccccc7c7ccccc7c6c5)ccc5ccccc45)cc3)n2)cc1]

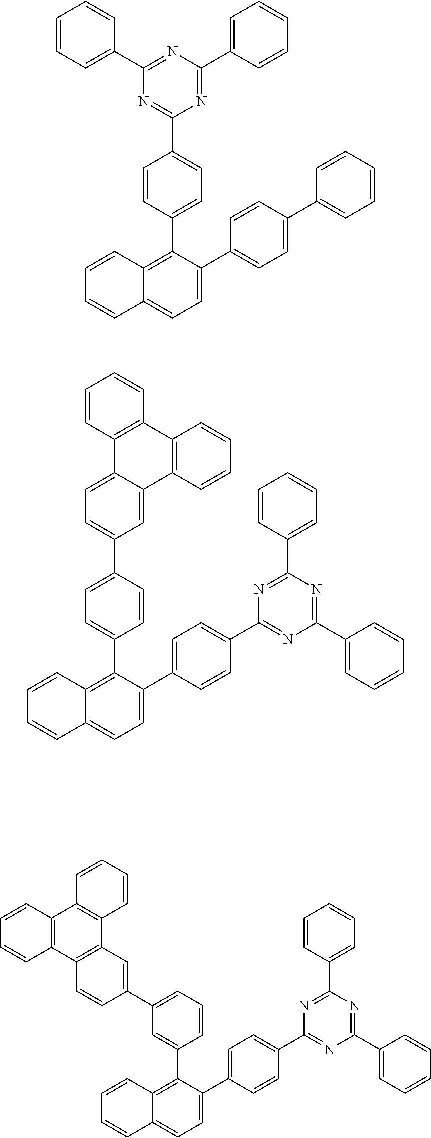 [SMILES: c1ccc(-c2ccc(-c3ccc4ccccc4c3-c3ccc(-c4nc(-c5ccccc5)nc(-c5ccccc5)n4)cc3)cc2)cc1 ; c1ccc(-c2nc(-c3ccccc3)nc(-c3ccc(-c4ccc5ccccc5c4-c4ccc(-c5ccc6c7ccccc7c7ccccc7c6c5)cc4)cc3)n2)cc1 ; c1ccc(-c2nc(-c3ccccc3)nc(-c3ccc(-c4ccc5ccccc5c4-c4cccc(-c5ccc6c7ccccc7c7ccccc7c6c5)c4)cc3)n2)cc1]

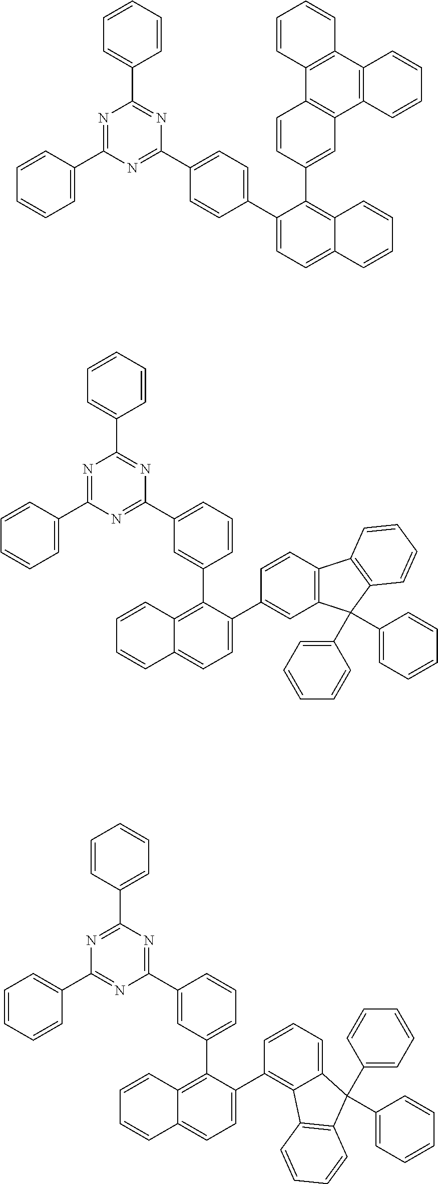 [SMILES: c1ccc(-c2nc(-c3ccccc3)nc(-c3ccc(-c4ccc5ccccc5c4-c4ccc5c6ccccc6c6ccccc6c5c4)cc3)n2)cc1 ; c1ccc(-c2nc(-c3ccccc3)nc(-c3cccc(-c4c(-c5ccc6c(c5)C(c5ccccc5)(c5ccccc5)c5ccccc5-6)ccc5ccccc45)c3)n2)cc1 ; c1ccc(-c2nc(-c3ccccc3)nc(-c3cccc(-c4c(-c5cccc6c5-c5ccccc5C6(c5ccccc5)c5ccccc5)ccc5ccccc45)c3)n2)cc1]

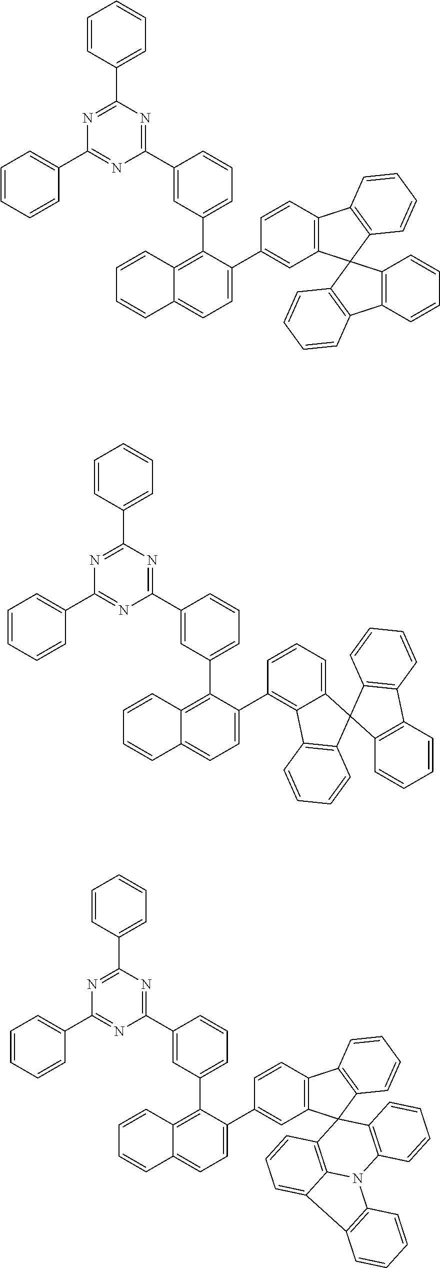 [SMILES: c1ccc(-c2nc(-c3ccccc3)nc(-c3cccc(-c4c(-c5ccc6c(c5)C5(c7ccccc7-c7ccccc75)c5ccccc5-6)ccc5ccccc45)c3)n2)cc1 ; c1ccc(-c2nc(-c3ccccc3)nc(-c3cccc(-c4c(-c5cccc6c5-c5ccccc5C65c6ccccc6-c6ccccc65)ccc5ccccc45)c3)n2)cc1 ; c1ccc(-c2nc(-c3ccccc3)nc(-c3cccc(-c4c(-c5ccc6c(c5)C5(c7ccccc7-6)c6ccccc6-n6c7ccccc7c7cccc5c76)ccc5ccccc45)c3)n2)cc1]

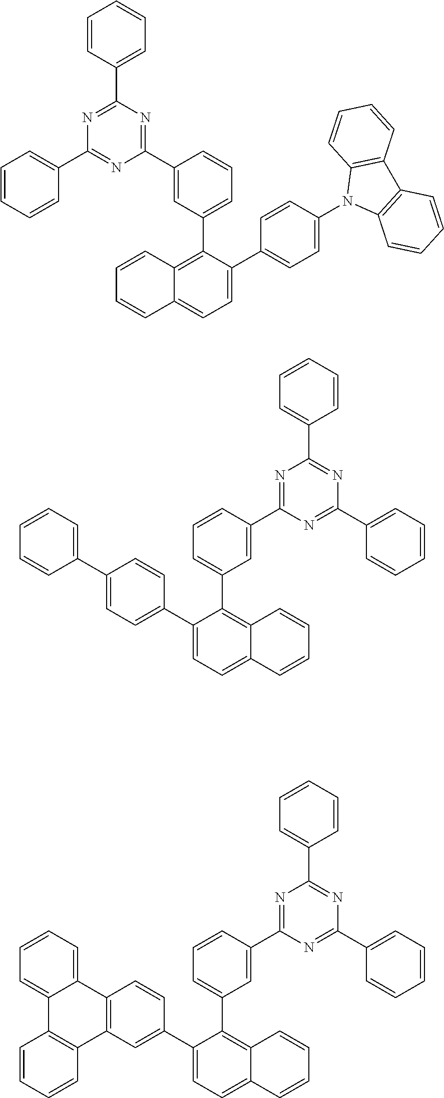 [SMILES: c1ccc(-c2nc(-c3ccccc3)nc(-c3cccc(-c4c(-c5ccc(-n6c7ccccc7c7ccccc76)cc5)ccc5ccccc45)c3)n2)cc1 ; c1ccc(-c2ccc(-c3ccc4ccccc4c3-c3cccc(-c4nc(-c5ccccc5)nc(-c5ccccc5)n4)c3)cc2)cc1 ; c1ccc(-c2nc(-c3ccccc3)nc(-c3cccc(-c4c(-c5ccc6c7ccccc7c7ccccc7c6c5)ccc5ccccc45)c3)n2)cc1]

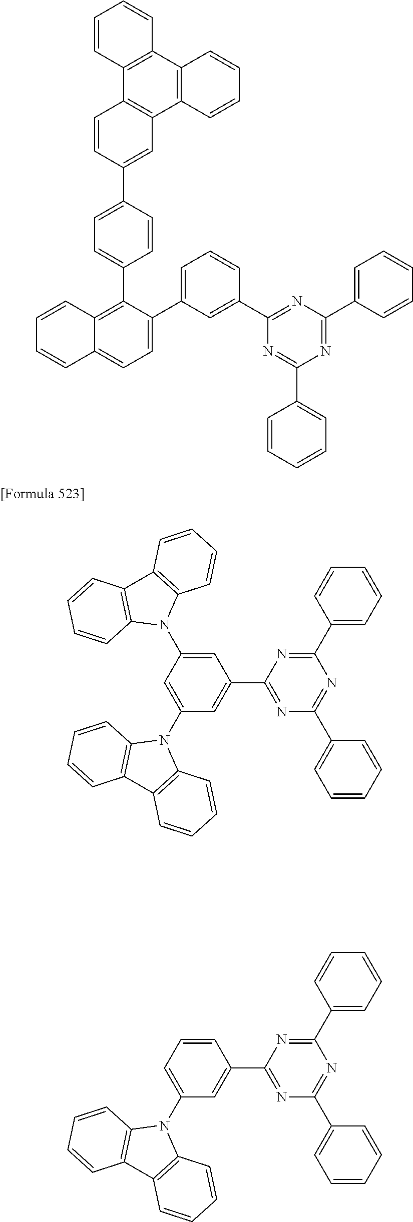 [SMILES: c1ccc(-c2nc(-c3ccccc3)nc(-c3cccc(-c4ccc5ccccc5c4-c4ccc(-c5ccc6c7ccccc7c7ccccc7c6c5)cc4)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1 ; c1ccc(-c2nc(-c3ccccc3)nc(-c3cccc(-n4c5ccccc5c5ccccc54)c3)n2)cc1]

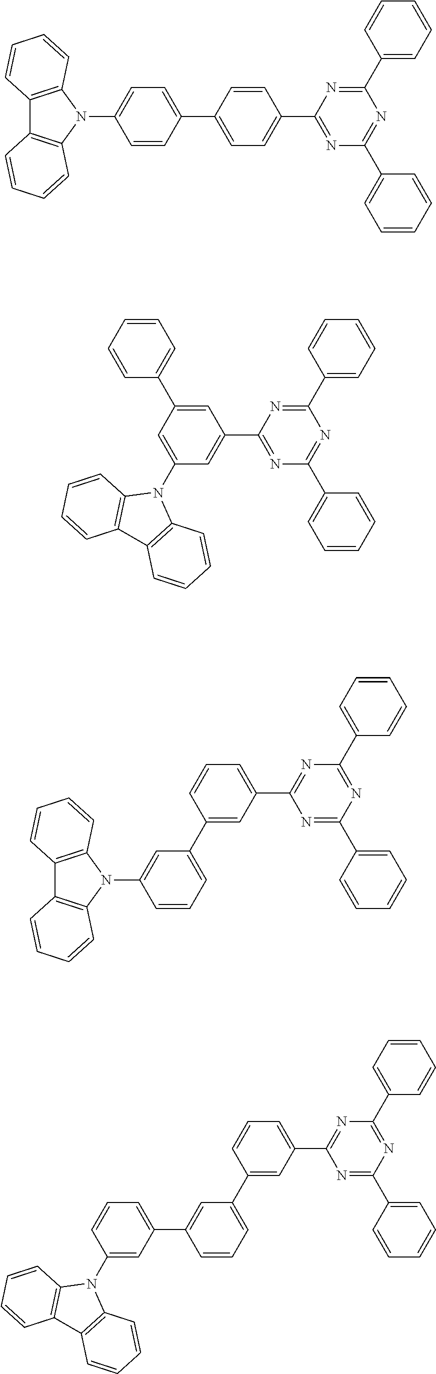 [SMILES: c1ccc(-c2nc(-c3ccccc3)nc(-c3ccc(-c4ccc(-n5c6ccccc6c6ccccc65)cc4)cc3)n2)cc1 ; c1ccc(-c2cc(-c3nc(-c4ccccc4)nc(-c4ccccc4)n3)cc(-n3c4ccccc4c4ccccc43)c2)cc1 ; c1ccc(-c2nc(-c3ccccc3)nc(-c3cccc(-c4cccc(-n5c6ccccc6c6ccccc65)c4)c3)n2)cc1 ; c1ccc(-c2nc(-c3ccccc3)nc(-c3cccc(-c4cccc(-c5cccc(-n6c7ccccc7c7ccccc76)c5)c4)c3)n2)cc1]

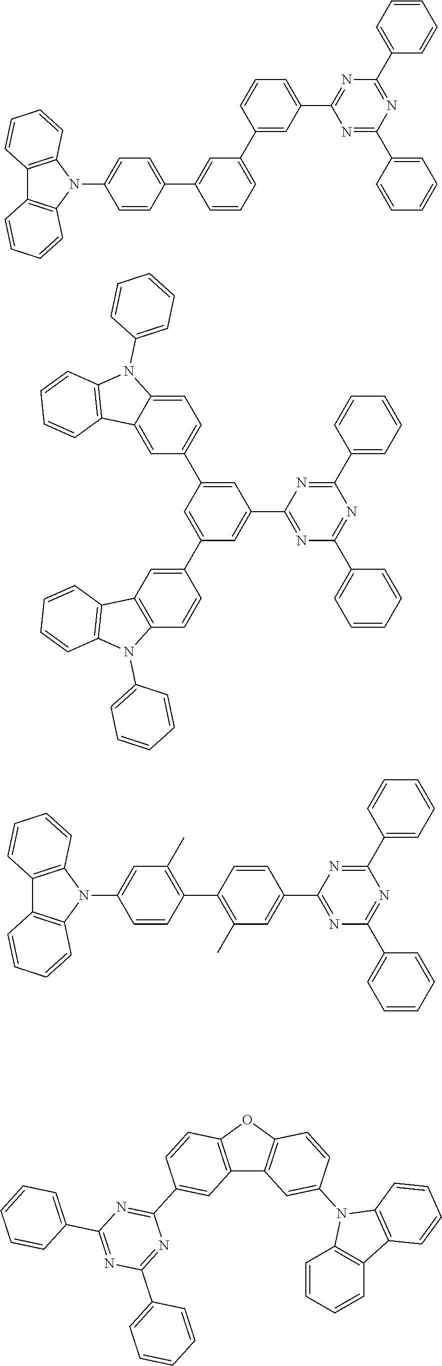 [SMILES: c1ccc(-c2nc(-c3ccccc3)nc(-c3cccc(-c4cccc(-c5ccc(-n6c7ccccc7c7ccccc76)cc5)c4)c3)n2)cc1 ; c1ccc(-c2nc(-c3ccccc3)nc(-c3cc(-c4ccc5c(c4)c4ccccc4n5-c4ccccc4)cc(-c4ccc5c(c4)c4ccccc4n5-c4ccccc4)c3)n2)cc1 ; Cc1cc(-c2nc(-c3ccccc3)nc(-c3ccccc3)n2)ccc1-c1ccc(-n2c3ccccc3c3ccccc32)cc1C ; c1ccc(-c2nc(-c3ccccc3)nc(-c3ccc4oc5ccc(-n6c7ccccc7c7ccccc76)cc5c4c3)n2)cc1]

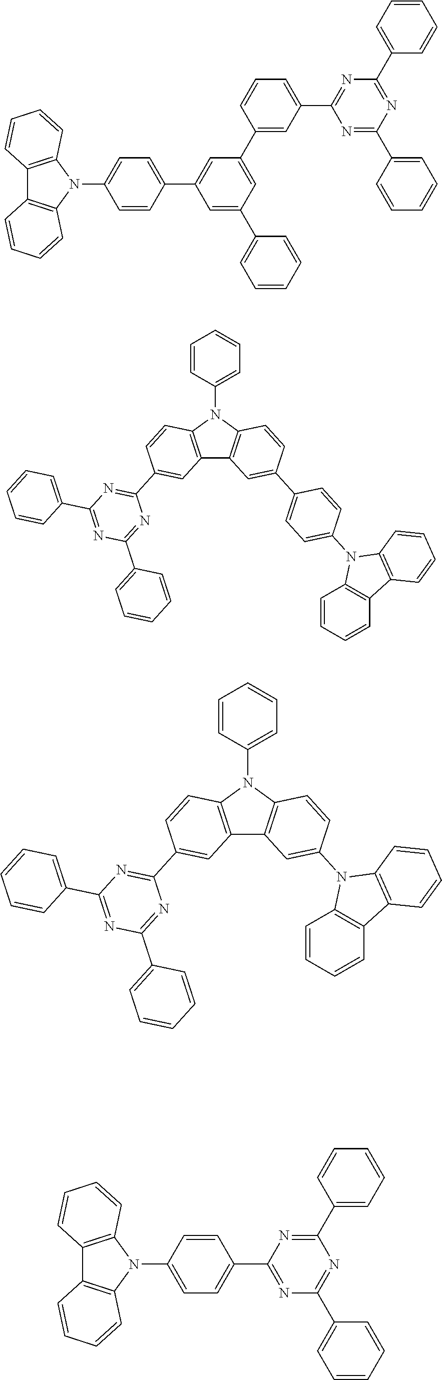 [SMILES: c1ccc(-c2cc(-c3ccc(-n4c5ccccc5c5ccccc54)cc3)cc(-c3cccc(-c4nc(-c5ccccc5)nc(-c5ccccc5)n4)c3)c2)cc1 ; c1ccc(-c2nc(-c3ccccc3)nc(-c3ccc4c(c3)c3cc(-c5ccc(-n6c7ccccc7c7ccccc76)cc5)ccc3n4-c3ccccc3)n2)cc1 ; c1ccc(-c2nc(-c3ccccc3)nc(-c3ccc4c(c3)c3cc(-n5c6ccccc6c6ccccc65)ccc3n4-c3ccccc3)n2)cc1 ; c1ccc(-c2nc(-c3ccccc3)nc(-c3ccc(-n4c5ccccc5c5ccccc54)cc3)n2)cc1]

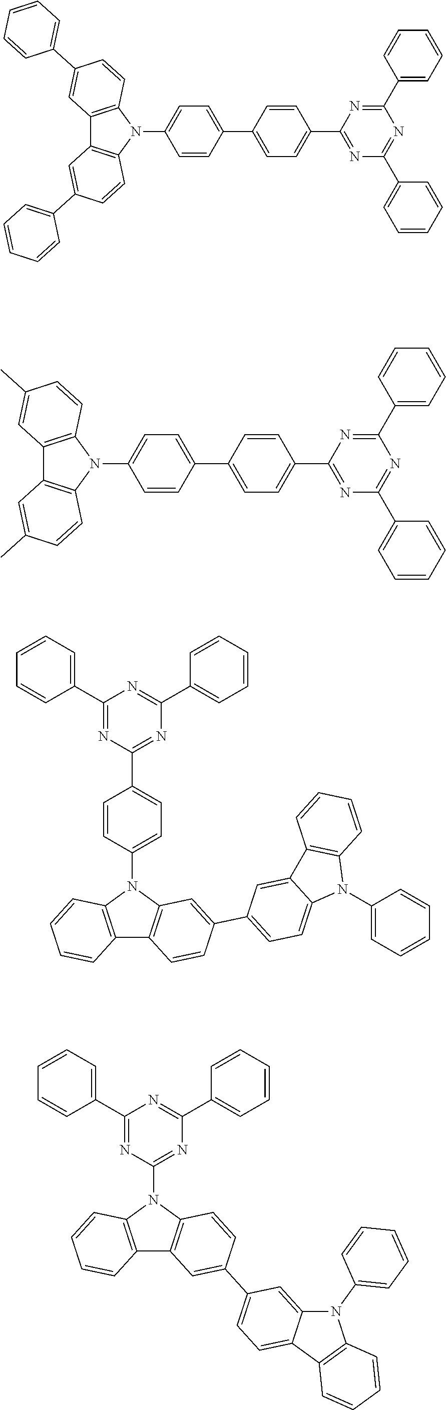 [SMILES: c1ccc(-c2ccc3c(c2)c2cc(-c4ccccc4)ccc2n3-c2ccc(-c3ccc(-c4nc(-c5ccccc5)nc(-c5ccccc5)n4)cc3)cc2)cc1 ; Cc1ccc2c(c1)c1cc(C)ccc1n2-c1ccc(-c2ccc(-c3nc(-c4ccccc4)nc(-c4ccccc4)n3)cc2)cc1 ; c1ccc(-c2nc(-c3ccccc3)nc(-c3ccc(-n4c5ccccc5c5ccc(-c6ccc7c(c6)c6ccccc6n7-c6ccccc6)cc54)cc3)n2)cc1 ; c1ccc(-c2nc(-c3ccccc3)nc(-n3c4ccccc4c4cc(-c5ccc6c7ccccc7n(-c7ccccc7)c6c5)ccc43)n2)cc1]

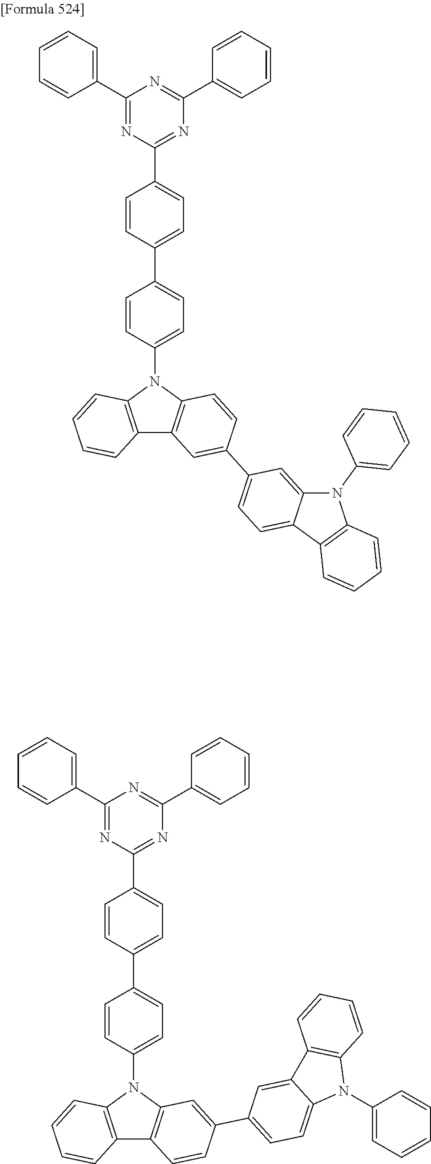 [SMILES: c1ccc(-c2nc(-c3ccccc3)nc(-c3ccc(-c4ccc(-n5c6ccccc6c6cc(-c7ccc8c9ccccc9n(-c9ccccc9)c8c7)ccc65)cc4)cc3)n2)cc1 ; c1ccc(-c2nc(-c3ccccc3)nc(-c3ccc(-c4ccc(-n5c6ccccc6c6ccc(-c7ccc8c(c7)c7ccccc7n8-c7ccccc7)cc65)cc4)cc3)n2)cc1]

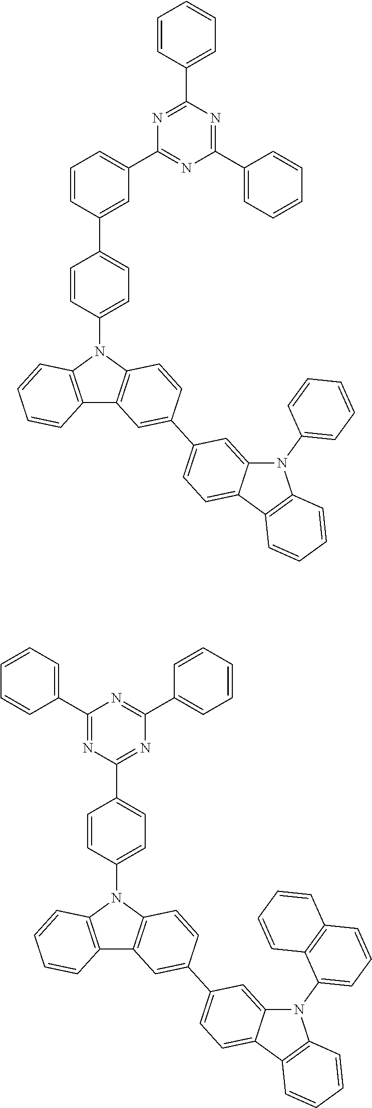 [SMILES: c1ccc(-c2nc(-c3ccccc3)nc(-c3cccc(-c4ccc(-n5c6ccccc6c6cc(-c7ccc8c9ccccc9n(-c9ccccc9)c8c7)ccc65)cc4)c3)n2)cc1 ; c1ccc(-c2nc(-c3ccccc3)nc(-c3ccc(-n4c5ccccc5c5cc(-c6ccc7c8ccccc8n(-c8cccc9ccccc89)c7c6)ccc54)cc3)n2)cc1]

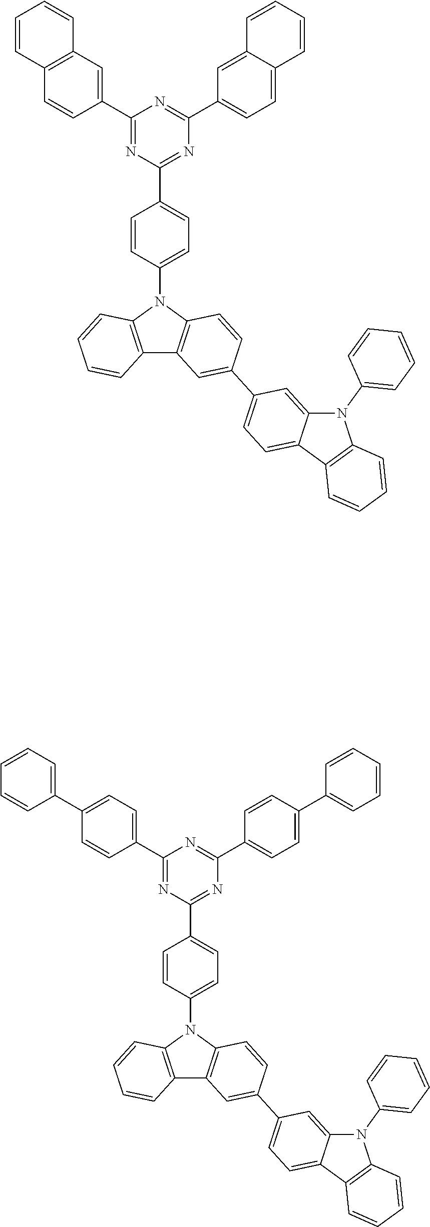 [SMILES: c1ccc(-n2c3ccccc3c3ccc(-c4ccc5c(c4)c4ccccc4n5-c4ccc(-c5nc(-c6ccc7ccccc7c6)nc(-c6ccc7ccccc7c6)n5)cc4)cc32)cc1 ; c1ccc(-c2ccc(-c3nc(-c4ccc(-c5ccccc5)cc4)nc(-c4ccc(-n5c6ccccc6c6cc(-c7ccc8c9ccccc9n(-c9ccccc9)c8c7)ccc65)cc4)n3)cc2)cc1]

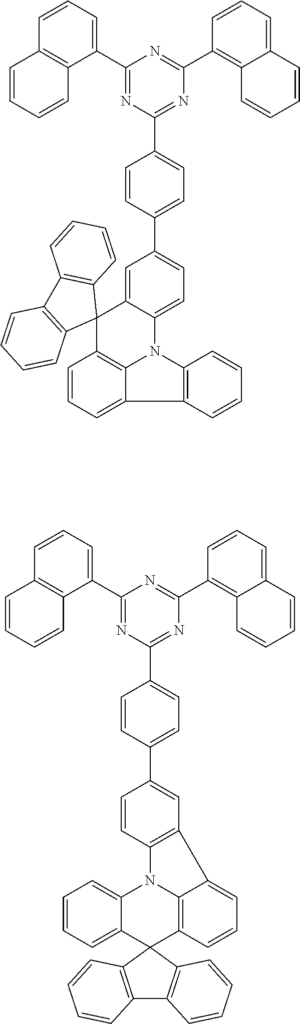 [SMILES: c1ccc2c(c1)-c1ccccc1C21c2cc(-c3ccc(-c4nc(-c5cccc6ccccc56)nc(-c5cccc6ccccc56)n4)cc3)ccc2-n2c3ccccc3c3cccc1c32 ; c1ccc2c(c1)-c1ccccc1C21c2ccccc2-n2c3ccc(-c4ccc(-c5nc(-c6cccc7ccccc67)nc(-c6cccc7ccccc67)n5)cc4)cc3c3cccc1c32]

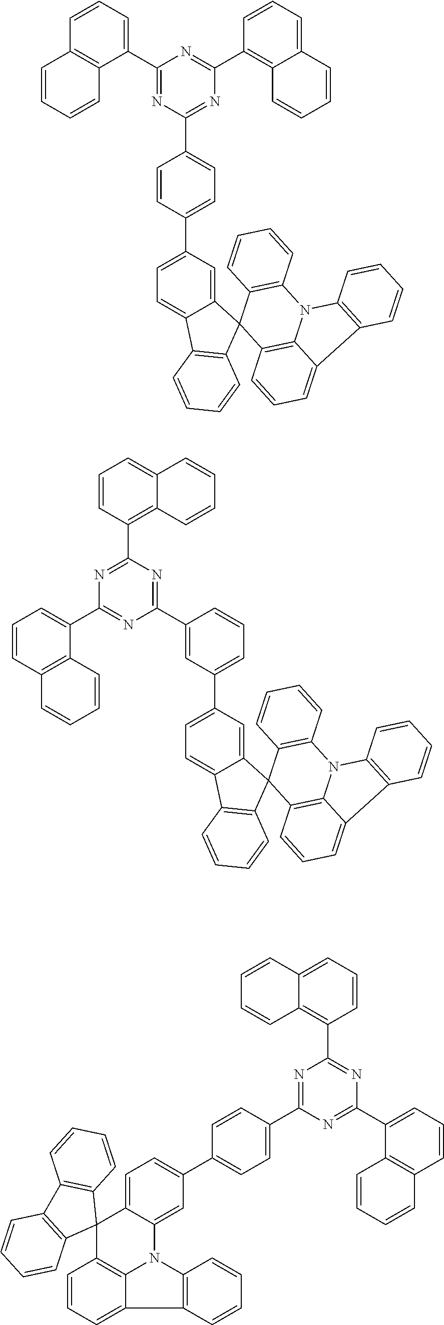 [SMILES: c1ccc2c(c1)-c1ccc(-c3ccc(-c4nc(-c5cccc6ccccc56)nc(-c5cccc6ccccc56)n4)cc3)cc1C21c2ccccc2-n2c3ccccc3c3cccc1c32 ; c1cc(-c2ccc3c(c2)C2(c4ccccc4-3)c3ccccc3-n3c4ccccc4c4cccc2c43)cc(-c2nc(-c3cccc4ccccc34)nc(-c3cccc4ccccc34)n2)c1 ; c1ccc2c(c1)-c1ccccc1C21c2ccc(-c3ccc(-c4nc(-c5cccc6ccccc56)nc(-c5cccc6ccccc56)n4)cc3)cc2-n2c3ccccc3c3cccc1c32]

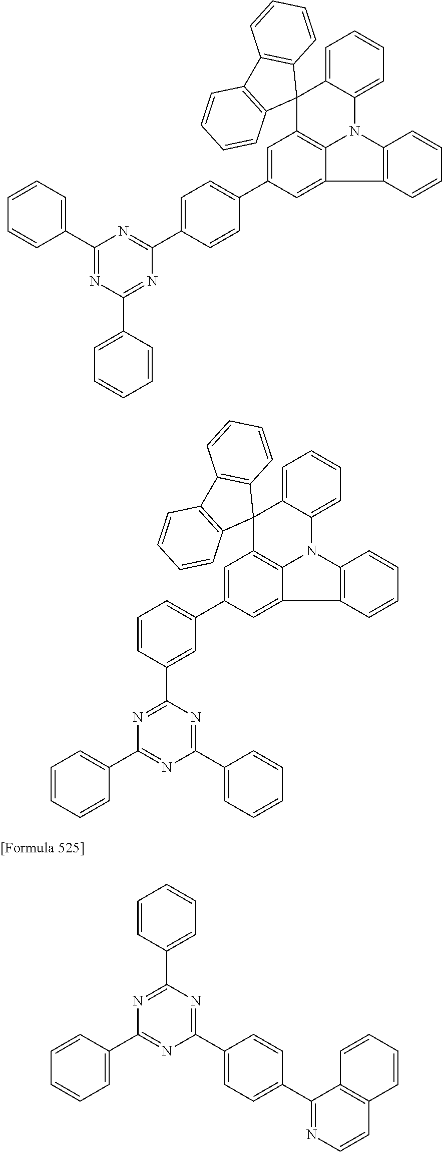 [SMILES: c1ccc(-c2nc(-c3ccccc3)nc(-c3ccc(-c4cc5c6c(c4)c4ccccc4n6-c4ccccc4C54c5ccccc5-c5ccccc54)cc3)n2)cc1 ; c1ccc(-c2nc(-c3ccccc3)nc(-c3cccc(-c4cc5c6c(c4)c4ccccc4n6-c4ccccc4C54c5ccccc5-c5ccccc54)c3)n2)cc1 ; c1ccc(-c2nc(-c3ccccc3)nc(-c3ccc(-c4nccc5ccccc45)cc3)n2)cc1]

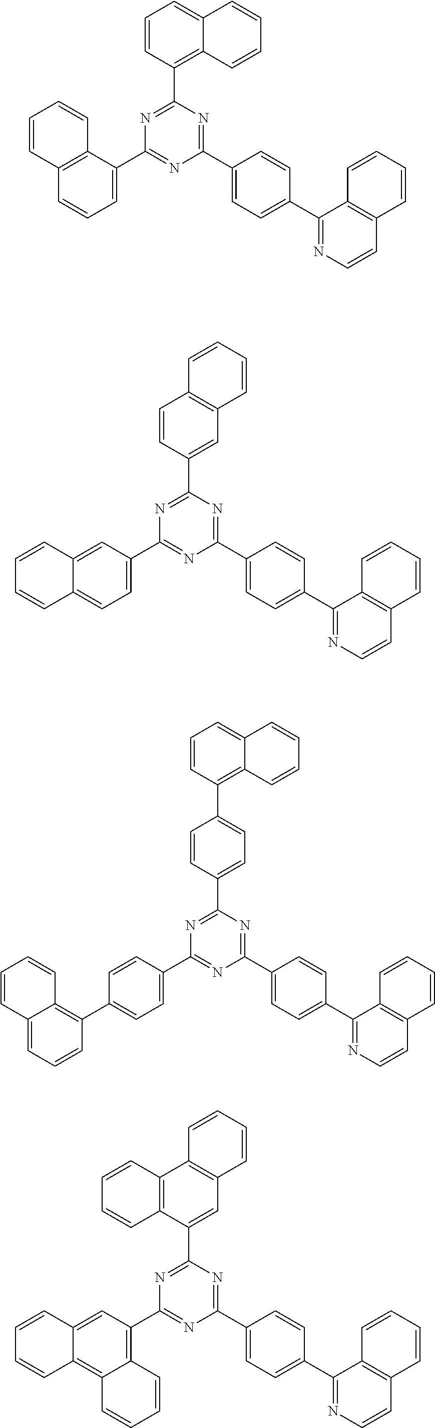 [SMILES: c1ccc2c(-c3nc(-c4ccc(-c5nccc6ccccc56)cc4)nc(-c4cccc5ccccc45)n3)cccc2c1 ; c1ccc2cc(-c3nc(-c4ccc(-c5nccc6ccccc56)cc4)nc(-c4ccc5ccccc5c4)n3)ccc2c1 ; c1ccc2c(-c3ccc(-c4nc(-c5ccc(-c6cccc7ccccc67)cc5)nc(-c5ccc(-c6nccc7ccccc67)cc5)n4)cc3)cccc2c1 ; c1ccc2c(-c3ccc(-c4nc(-c5cc6ccccc6c6ccccc56)nc(-c5cc6ccccc6c6ccccc56)n4)cc3)nccc2c1]

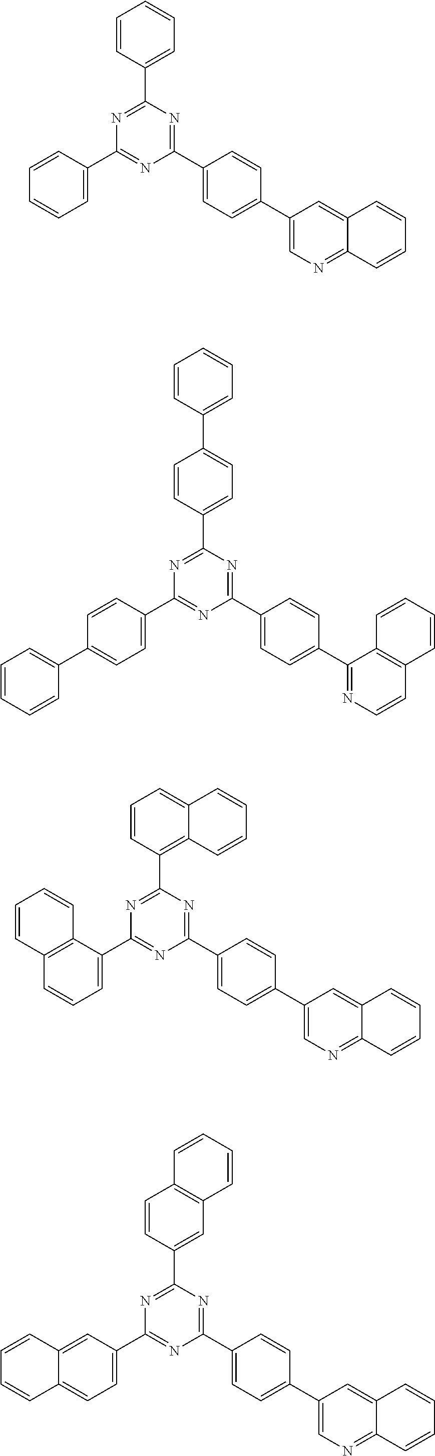 [SMILES: c1ccc(-c2nc(-c3ccccc3)nc(-c3ccc(-c4cnc5ccccc5c4)cc3)n2)cc1 ; c1ccc(-c2ccc(-c3nc(-c4ccc(-c5ccccc5)cc4)nc(-c4ccc(-c5nccc6ccccc56)cc4)n3)cc2)cc1 ; c1ccc2ncc(-c3ccc(-c4nc(-c5cccc6ccccc56)nc(-c5cccc6ccccc56)n4)cc3)cc2c1 ; c1ccc2cc(-c3nc(-c4ccc(-c5cnc6ccccc6c5)cc4)nc(-c4ccc5ccccc5c4)n3)ccc2c1]

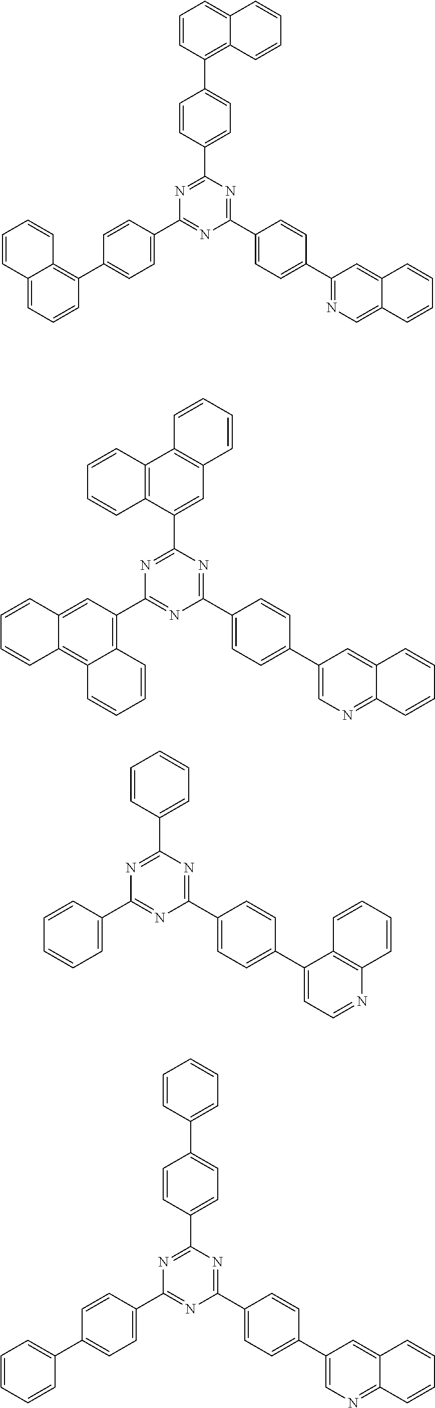 [SMILES: c1ccc2cc(-c3ccc(-c4nc(-c5ccc(-c6cccc7ccccc67)cc5)nc(-c5ccc(-c6cccc7ccccc67)cc5)n4)cc3)ncc2c1 ; c1ccc2ncc(-c3ccc(-c4nc(-c5cc6ccccc6c6ccccc56)nc(-c5cc6ccccc6c6ccccc56)n4)cc3)cc2c1 ; c1ccc(-c2nc(-c3ccccc3)nc(-c3ccc(-c4ccnc5ccccc45)cc3)n2)cc1 ; c1ccc(-c2ccc(-c3nc(-c4ccc(-c5ccccc5)cc4)nc(-c4ccc(-c5cnc6ccccc6c5)cc4)n3)cc2)cc1]

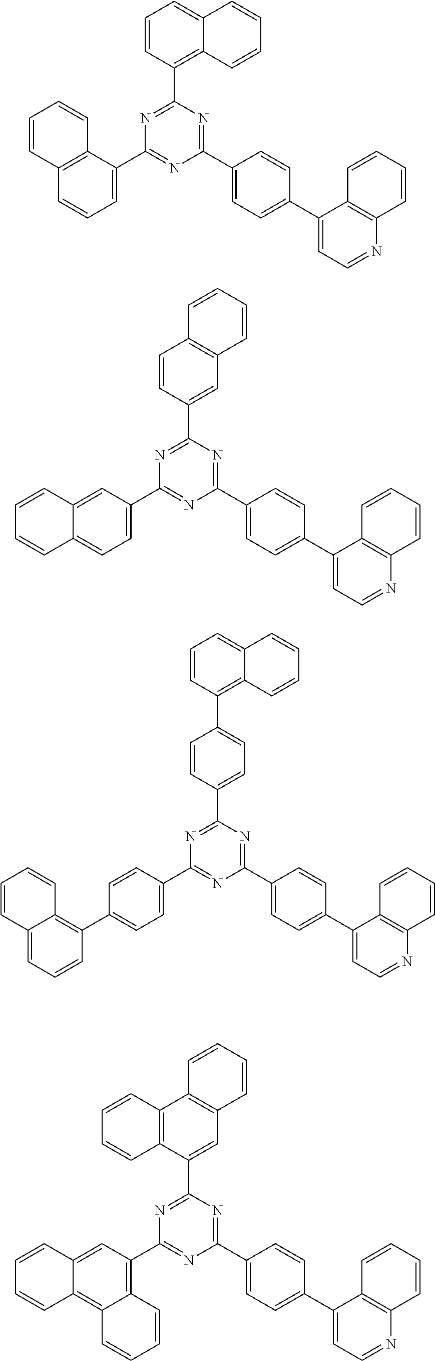 [SMILES: c1ccc2c(-c3nc(-c4ccc(-c5ccnc6ccccc56)cc4)nc(-c4cccc5ccccc45)n3)cccc2c1 ; c1ccc2cc(-c3nc(-c4ccc(-c5ccnc6ccccc56)cc4)nc(-c4ccc5ccccc5c4)n3)ccc2c1 ; c1ccc2c(-c3ccc(-c4nc(-c5ccc(-c6cccc7ccccc67)cc5)nc(-c5ccc(-c6ccnc7ccccc67)cc5)n4)cc3)cccc2c1 ; c1ccc2c(c1)cc(-c1nc(-c3ccc(-c4ccnc5ccccc45)cc3)nc(-c3cc4ccccc4c4ccccc34)n1)c1ccccc12]

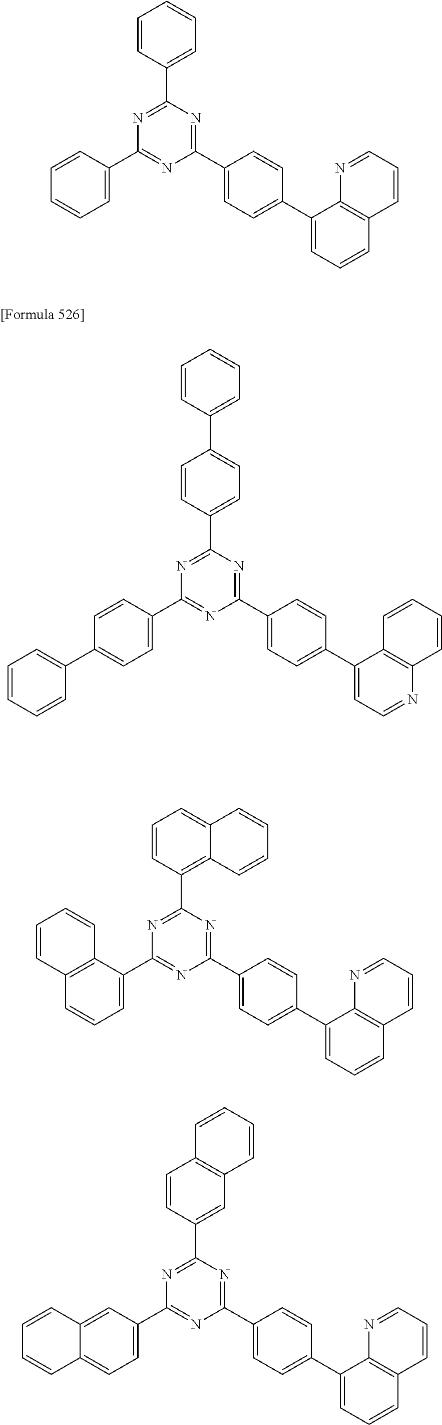 [SMILES: c1ccc(-c2nc(-c3ccccc3)nc(-c3ccc(-c4cccc5cccnc45)cc3)n2)cc1 ; c1ccc(-c2ccc(-c3nc(-c4ccc(-c5ccccc5)cc4)nc(-c4ccc(-c5ccnc6ccccc56)cc4)n3)cc2)cc1 ; c1ccc2c(-c3nc(-c4ccc(-c5cccc6cccnc56)cc4)nc(-c4cccc5ccccc45)n3)cccc2c1 ; c1ccc2cc(-c3nc(-c4ccc(-c5cccc6cccnc56)cc4)nc(-c4ccc5ccccc5c4)n3)ccc2c1]

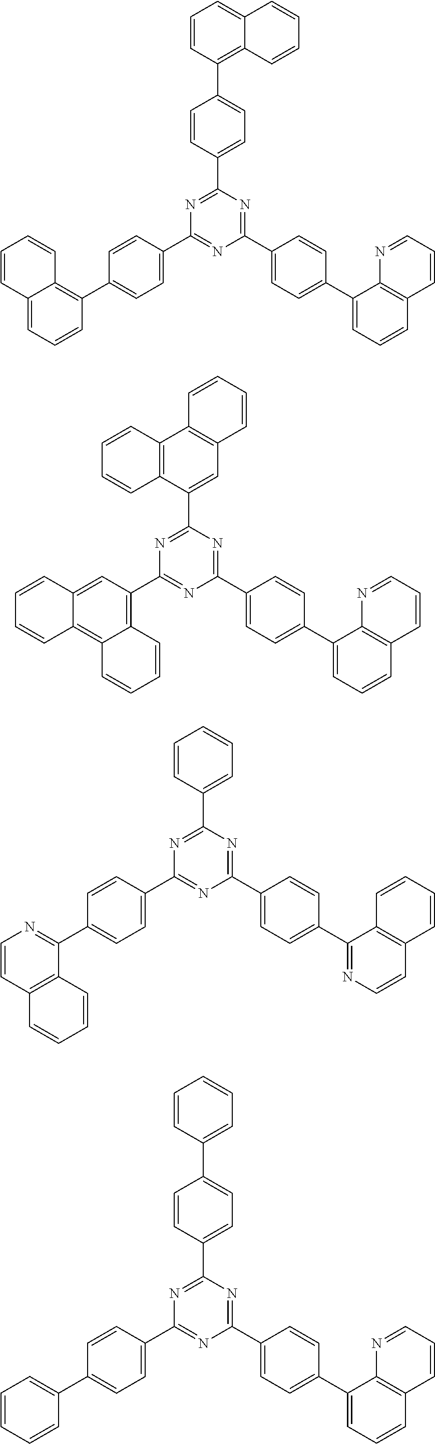 [SMILES: c1ccc2c(-c3ccc(-c4nc(-c5ccc(-c6cccc7ccccc67)cc5)nc(-c5ccc(-c6cccc7cccnc67)cc5)n4)cc3)cccc2c1 ; c1cnc2c(-c3ccc(-c4nc(-c5cc6ccccc6c6ccccc56)nc(-c5cc6ccccc6c6ccccc56)n4)cc3)cccc2c1 ; c1ccc(-c2nc(-c3ccc(-c4nccc5ccccc45)cc3)nc(-c3ccc(-c4nccc5ccccc45)cc3)n2)cc1 ; c1ccc(-c2ccc(-c3nc(-c4ccc(-c5ccccc5)cc4)nc(-c4ccc(-c5cccc6cccnc56)cc4)n3)cc2)cc1]

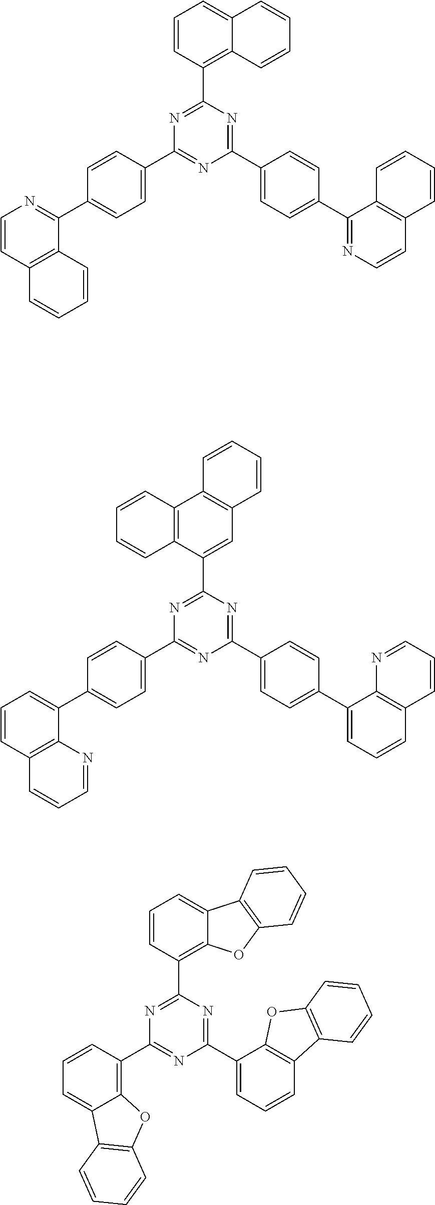 [SMILES: c1ccc2c(-c3nc(-c4ccc(-c5nccc6ccccc56)cc4)nc(-c4ccc(-c5nccc6ccccc56)cc4)n3)cccc2c1 ; c1cnc2c(-c3ccc(-c4nc(-c5ccc(-c6cccc7cccnc67)cc5)nc(-c5cc6ccccc6c6ccccc56)n4)cc3)cccc2c1 ; c1ccc2c(c1)oc1c(-c3nc(-c4cccc5c4oc4ccccc45)nc(-c4cccc5c4oc4ccccc45)n3)cccc12]

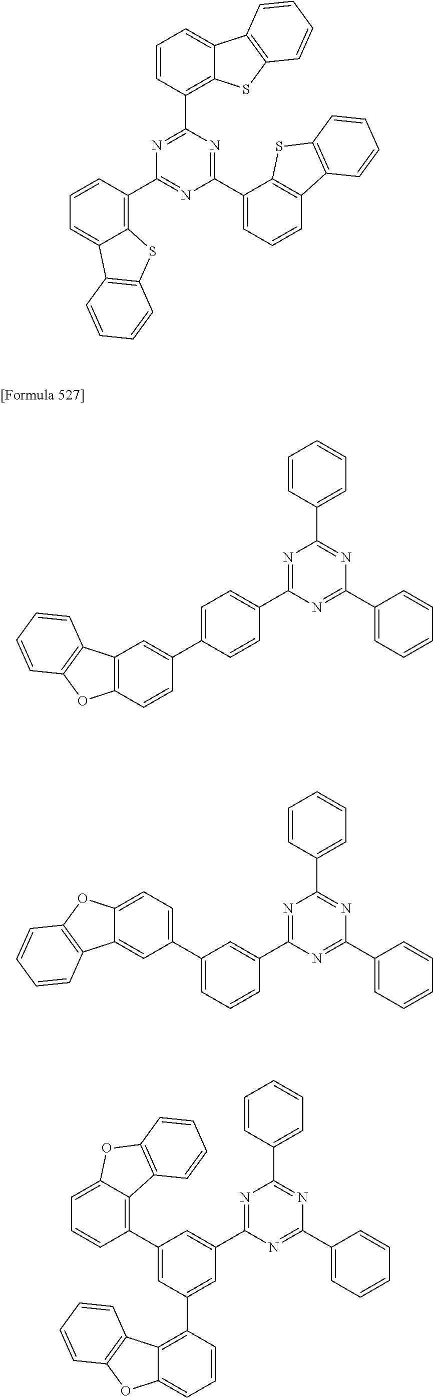 [SMILES: c1ccc2c(c1)sc1c(-c3nc(-c4cccc5c4sc4ccccc45)nc(-c4cccc5c4sc4ccccc45)n3)cccc12 ; c1ccc(-c2nc(-c3ccccc3)nc(-c3ccc(-c4ccc5oc6ccccc6c5c4)cc3)n2)cc1 ; c1ccc(-c2nc(-c3ccccc3)nc(-c3cccc(-c4ccc5oc6ccccc6c5c4)c3)n2)cc1 ; c1ccc(-c2nc(-c3ccccc3)nc(-c3cc(-c4cccc5oc6ccccc6c45)cc(-c4cccc5oc6ccccc6c45)c3)n2)cc1]

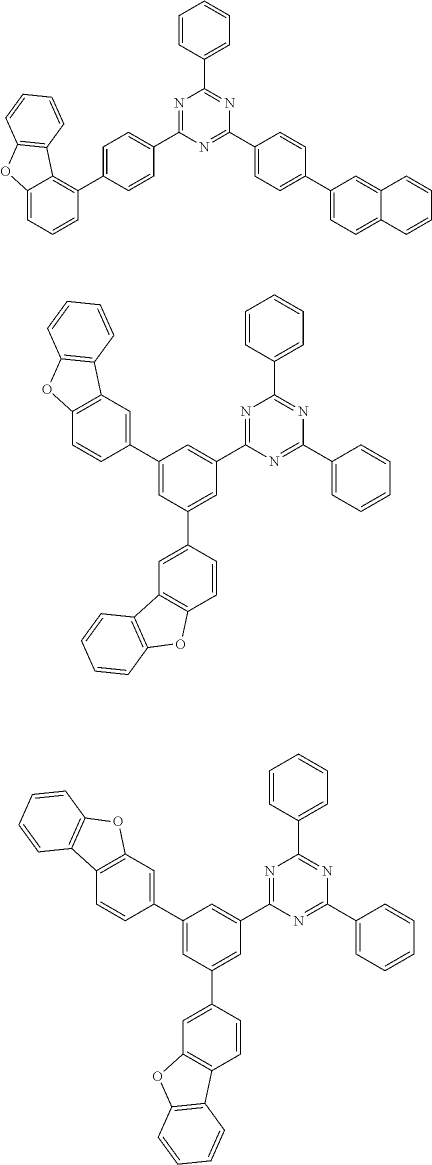 [SMILES: c1ccc(-c2nc(-c3ccc(-c4ccc5ccccc5c4)cc3)nc(-c3ccc(-c4cccc5oc6ccccc6c45)cc3)n2)cc1 ; c1ccc(-c2nc(-c3ccccc3)nc(-c3cc(-c4ccc5oc6ccccc6c5c4)cc(-c4ccc5oc6ccccc6c5c4)c3)n2)cc1 ; c1ccc(-c2nc(-c3ccccc3)nc(-c3cc(-c4ccc5c(c4)oc4ccccc45)cc(-c4ccc5c(c4)oc4ccccc45)c3)n2)cc1]

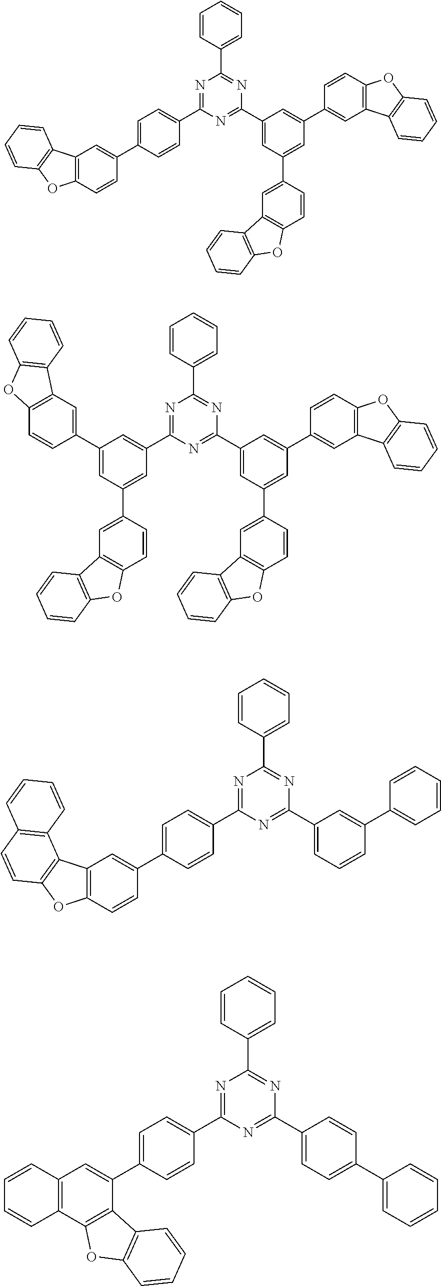 [SMILES: c1ccc(-c2nc(-c3ccc(-c4ccc5oc6ccccc6c5c4)cc3)nc(-c3cc(-c4ccc5oc6ccccc6c5c4)cc(-c4ccc5oc6ccccc6c5c4)c3)n2)cc1 ; c1ccc(-c2nc(-c3cc(-c4ccc5oc6ccccc6c5c4)cc(-c4ccc5oc6ccccc6c5c4)c3)nc(-c3cc(-c4ccc5oc6ccccc6c5c4)cc(-c4ccc5oc6ccccc6c5c4)c3)n2)cc1 ; c1ccc(-c2cccc(-c3nc(-c4ccccc4)nc(-c4ccc(-c5ccc6oc7ccc8ccccc8c7c6c5)cc4)n3)c2)cc1 ; c1ccc(-c2ccc(-c3nc(-c4ccccc4)nc(-c4ccc(-c5cc6ccccc6c6oc7ccccc7c56)cc4)n3)cc2)cc1]

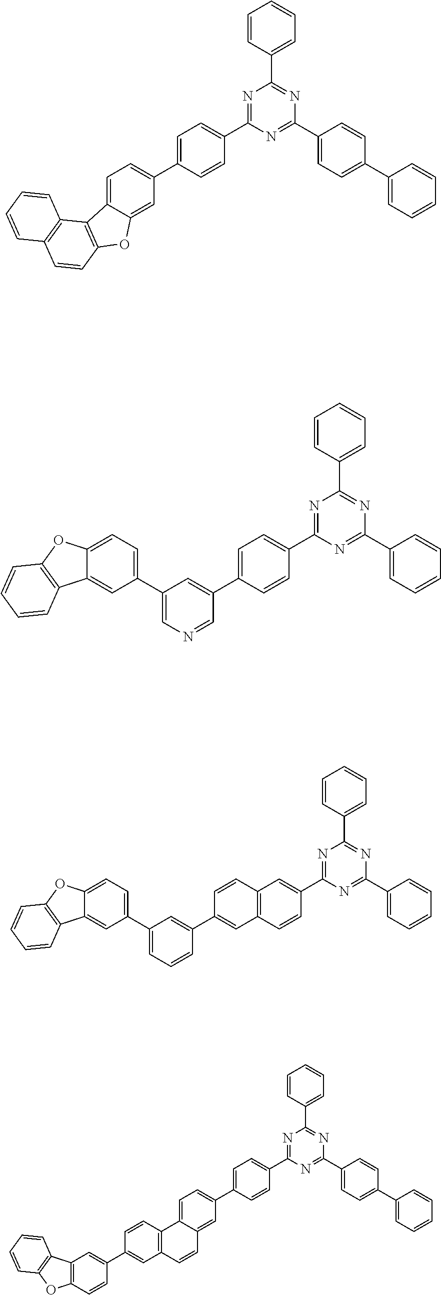 [SMILES: c1ccc(-c2ccc(-c3nc(-c4ccccc4)nc(-c4ccc(-c5ccc6c(c5)oc5ccc7ccccc7c56)cc4)n3)cc2)cc1 ; c1ccc(-c2nc(-c3ccccc3)nc(-c3ccc(-c4cncc(-c5ccc6oc7ccccc7c6c5)c4)cc3)n2)cc1 ; c1ccc(-c2nc(-c3ccccc3)nc(-c3ccc4cc(-c5cccc(-c6ccc7oc8ccccc8c7c6)c5)ccc4c3)n2)cc1 ; c1ccc(-c2ccc(-c3nc(-c4ccccc4)nc(-c4ccc(-c5ccc6c(ccc7cc(-c8ccc9oc%10ccccc%10c9c8)ccc76)c5)cc4)n3)cc2)cc1]

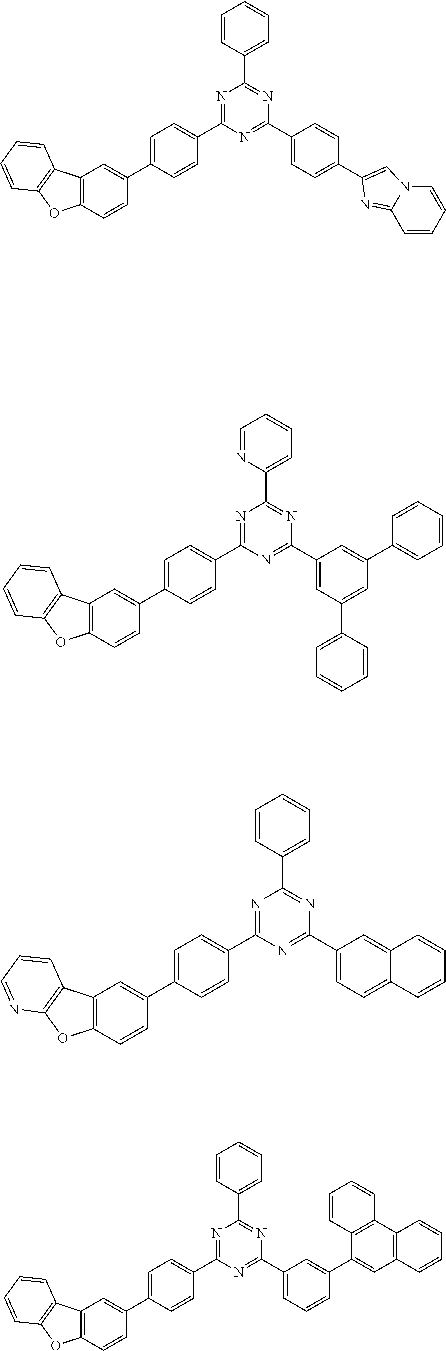 [SMILES: c1ccc(-c2nc(-c3ccc(-c4ccc5oc6ccccc6c5c4)cc3)nc(-c3ccc(-c4cn5ccccc5n4)cc3)n2)cc1 ; c1ccc(-c2cc(-c3ccccc3)cc(-c3nc(-c4ccc(-c5ccc6oc7ccccc7c6c5)cc4)nc(-c4ccccn4)n3)c2)cc1 ; c1ccc(-c2nc(-c3ccc(-c4ccc5oc6ncccc6c5c4)cc3)nc(-c3ccc4ccccc4c3)n2)cc1 ; c1ccc(-c2nc(-c3ccc(-c4ccc5oc6ccccc6c5c4)cc3)nc(-c3cccc(-c4cc5ccccc5c5ccccc45)c3)n2)cc1]

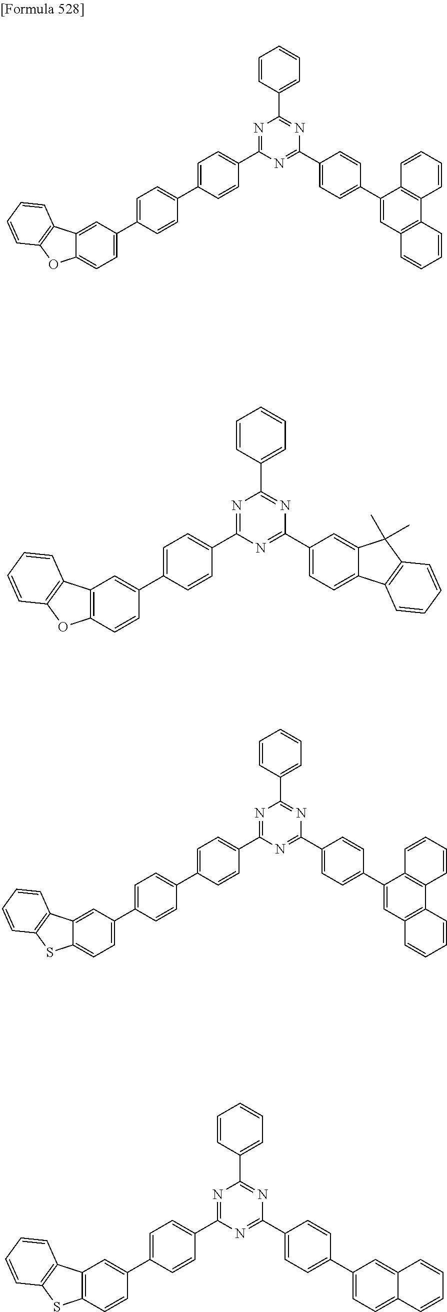 [SMILES: c1ccc(-c2nc(-c3ccc(-c4ccc(-c5ccc6oc7ccccc7c6c5)cc4)cc3)nc(-c3ccc(-c4cc5ccccc5c5ccccc45)cc3)n2)cc1 ; CC1(C)c2ccccc2-c2ccc(-c3nc(-c4ccccc4)nc(-c4ccc(-c5ccc6oc7ccccc7c6c5)cc4)n3)cc21 ; c1ccc(-c2nc(-c3ccc(-c4ccc(-c5ccc6sc7ccccc7c6c5)cc4)cc3)nc(-c3ccc(-c4cc5ccccc5c5ccccc45)cc3)n2)cc1 ; c1ccc(-c2nc(-c3ccc(-c4ccc5ccccc5c4)cc3)nc(-c3ccc(-c4ccc5sc6ccccc6c5c4)cc3)n2)cc1]

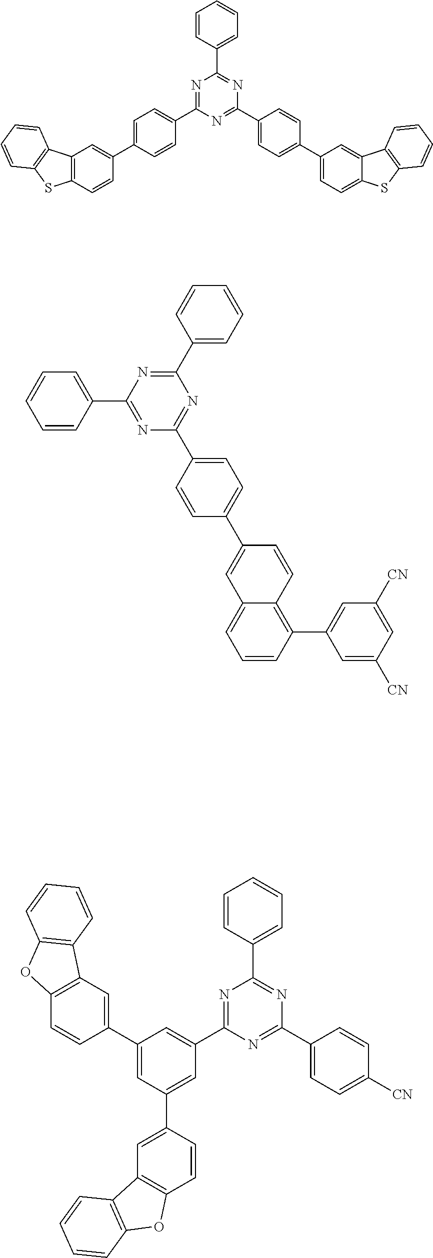 [SMILES: c1ccc(-c2nc(-c3ccc(-c4ccc5sc6ccccc6c5c4)cc3)nc(-c3ccc(-c4ccc5sc6ccccc6c5c4)cc3)n2)cc1 ; N#Cc1cc(C#N)cc(-c2cccc3cc(-c4ccc(-c5nc(-c6ccccc6)nc(-c6ccccc6)n5)cc4)ccc23)c1 ; N#Cc1ccc(-c2nc(-c3ccccc3)nc(-c3cc(-c4ccc5oc6ccccc6c5c4)cc(-c4ccc5oc6ccccc6c5c4)c3)n2)cc1]

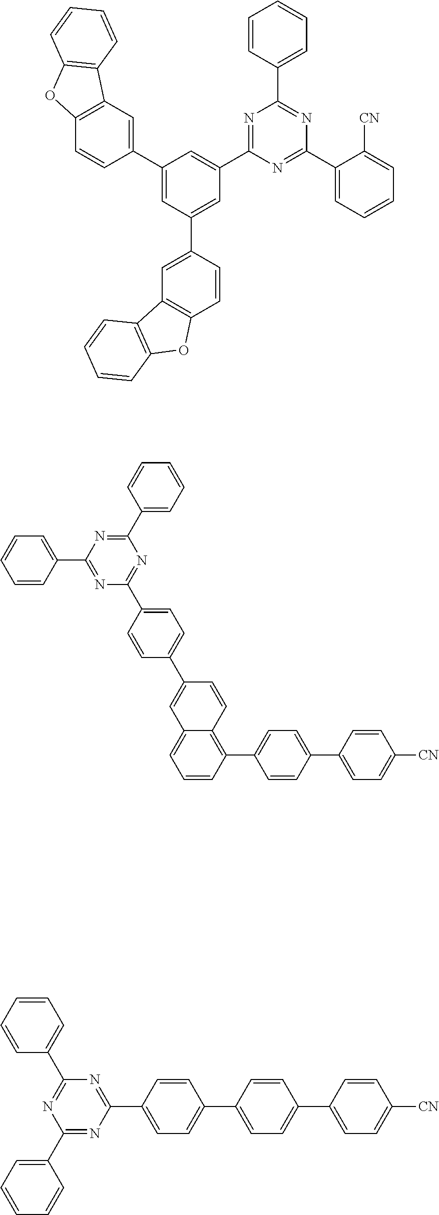 [SMILES: N#Cc1ccccc1-c1nc(-c2ccccc2)nc(-c2cc(-c3ccc4oc5ccccc5c4c3)cc(-c3ccc4oc5ccccc5c4c3)c2)n1 ; N#Cc1ccc(-c2ccc(-c3cccc4cc(-c5ccc(-c6nc(-c7ccccc7)nc(-c7ccccc7)n6)cc5)ccc34)cc2)cc1 ; N#Cc1ccc(-c2ccc(-c3ccc(-c4nc(-c5ccccc5)nc(-c5ccccc5)n4)cc3)cc2)cc1]

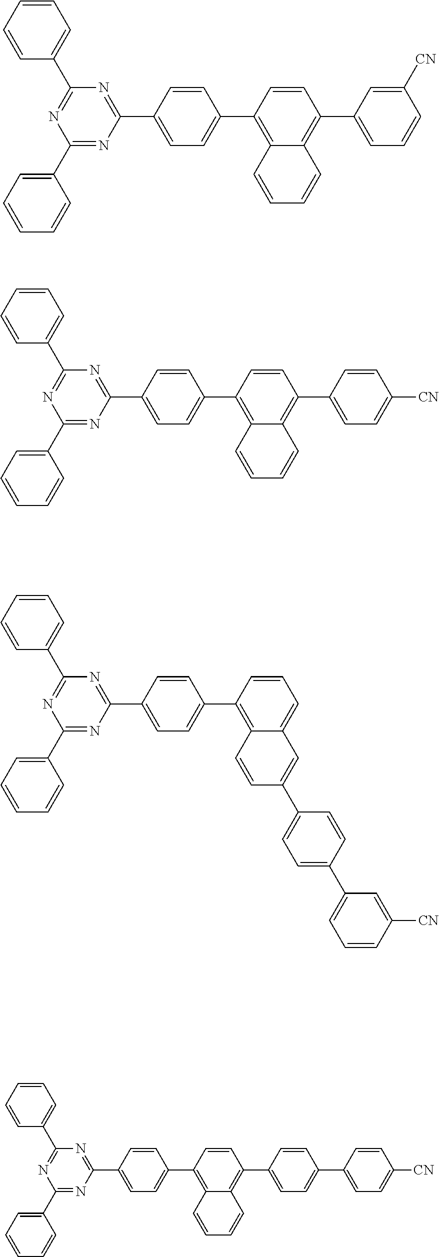 [SMILES: N#Cc1cccc(-c2ccc(-c3ccc(-c4nc(-c5ccccc5)nc(-c5ccccc5)n4)cc3)c3ccccc23)c1 ; N#Cc1ccc(-c2ccc(-c3ccc(-c4nc(-c5ccccc5)nc(-c5ccccc5)n4)cc3)c3ccccc23)cc1 ; N#Cc1cccc(-c2ccc(-c3ccc4c(-c5ccc(-c6nc(-c7ccccc7)nc(-c7ccccc7)n6)cc5)cccc4c3)cc2)c1 ; N#Cc1ccc(-c2ccc(-c3ccc(-c4ccc(-c5nc(-c6ccccc6)nc(-c6ccccc6)n5)cc4)c4ccccc34)cc2)cc1]

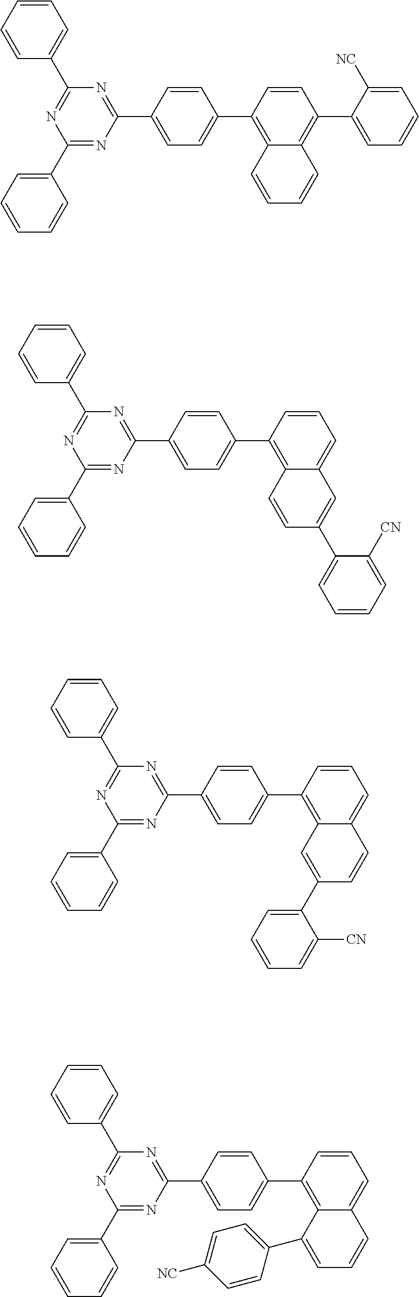 [SMILES: N#Cc1ccccc1-c1ccc(-c2ccc(-c3nc(-c4ccccc4)nc(-c4ccccc4)n3)cc2)c2ccccc12 ; N#Cc1ccccc1-c1ccc2c(-c3ccc(-c4nc(-c5ccccc5)nc(-c5ccccc5)n4)cc3)cccc2c1 ; N#Cc1ccccc1-c1ccc2cccc(-c3ccc(-c4nc(-c5ccccc5)nc(-c5ccccc5)n4)cc3)c2c1 ; N#Cc1ccc(-c2cccc3cccc(-c4ccc(-c5nc(-c6ccccc6)nc(-c6ccccc6)n5)cc4)c23)cc1]

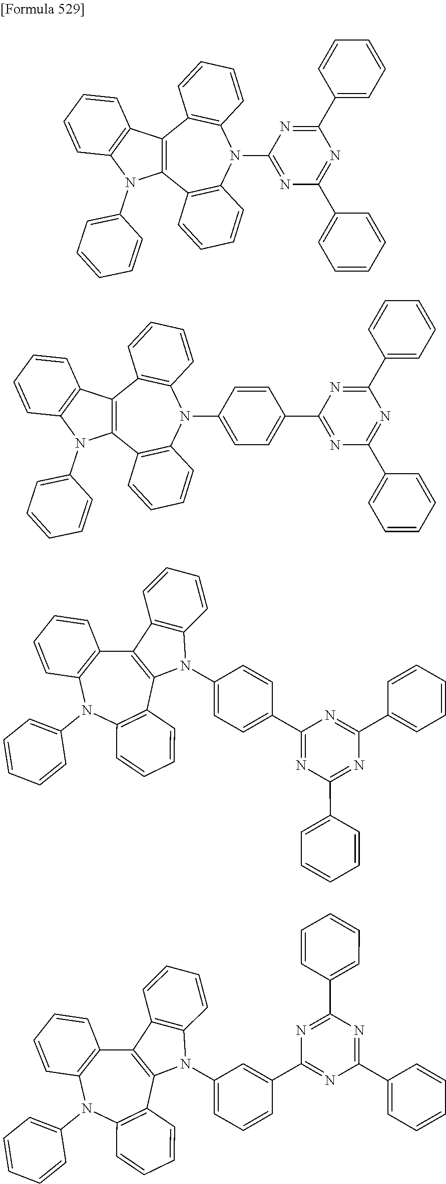

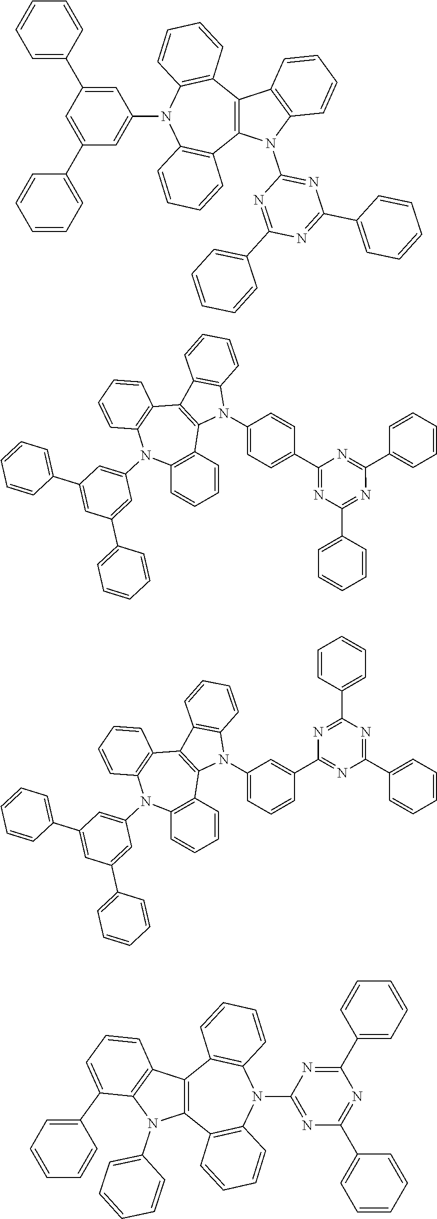

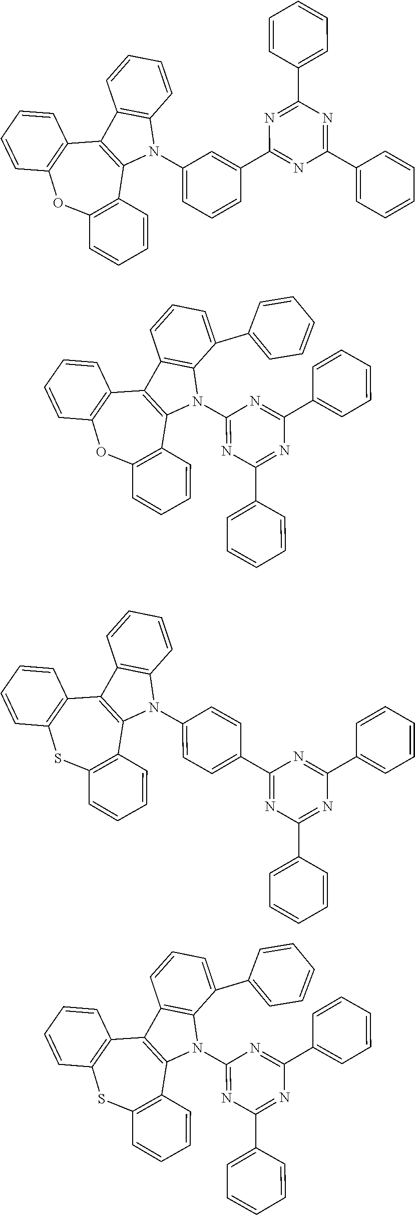

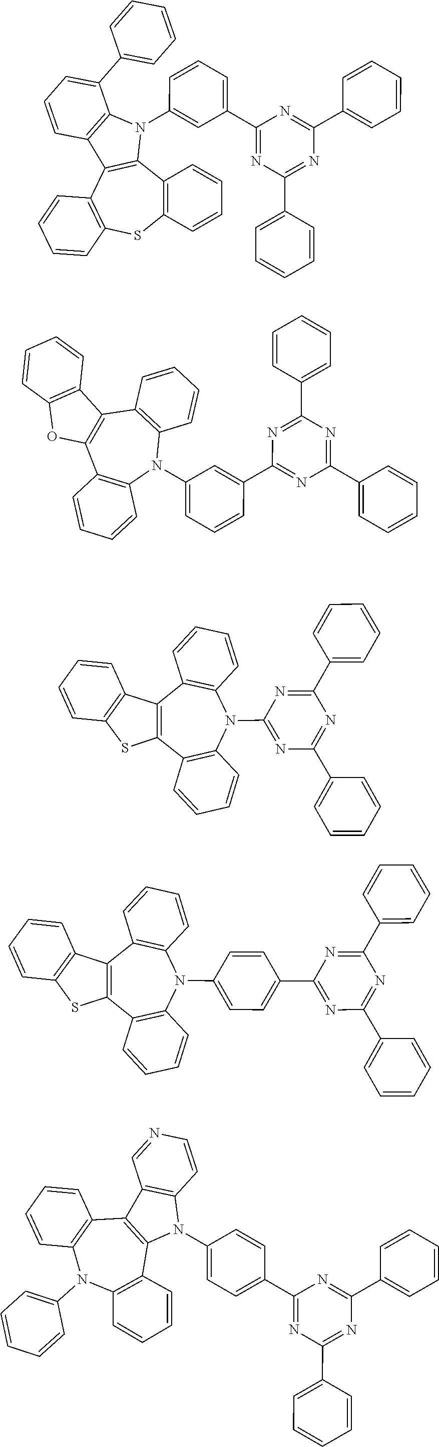

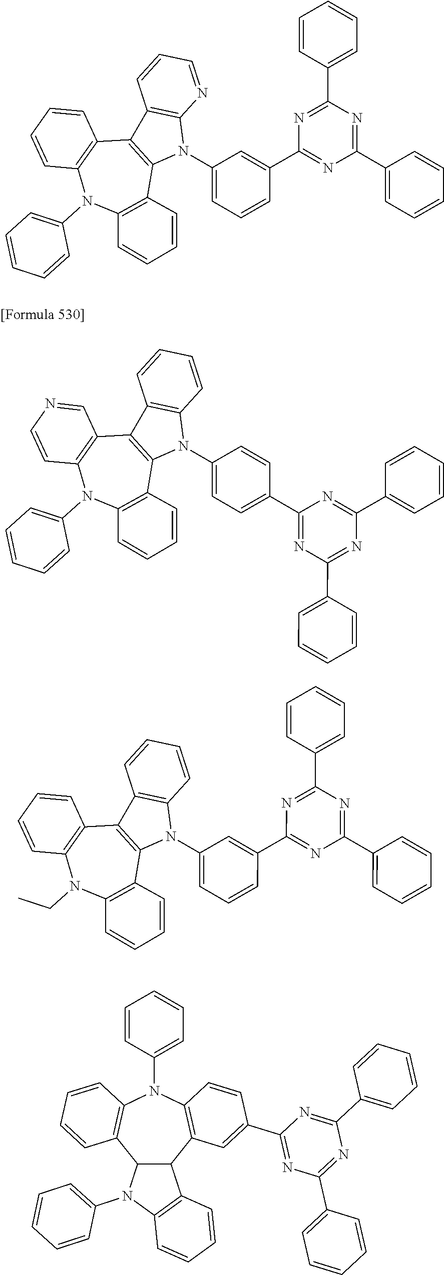

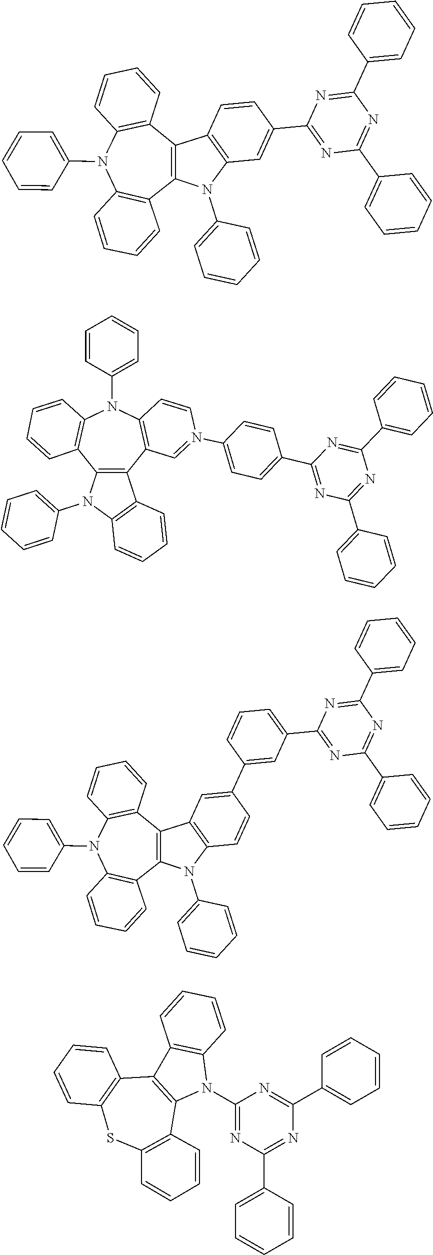

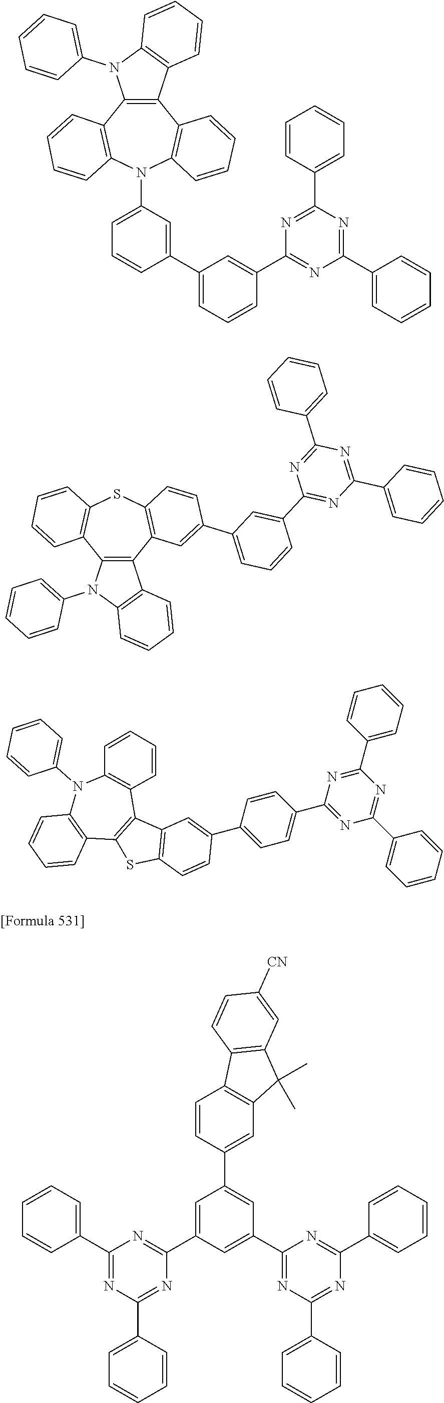 [SMILES: c1ccc(-c2nc(-c3ccccc3)nc(-c3cccc(-c4cccc(N5c6ccccc6-c6c(n(-c7ccccc7)c7ccccc67)-c6ccccc65)c4)c3)n2)cc1 ; c1ccc(-c2nc(-c3ccccc3)nc(-c3cccc(-c4ccc5c(c4)-c4c(n(-c6ccccc6)c6ccccc46)-c4ccccc4S5)c3)n2)cc1 ; c1ccc(-c2nc(-c3ccccc3)nc(-c3ccc(-c4ccc5sc6c(c5c4)-c4ccccc4N(c4ccccc4)c4ccccc4-6)cc3)n2)cc1 ; CC1(C)c2cc(C#N)ccc2-c2ccc(-c3cc(-c4nc(-c5ccccc5)nc(-c5ccccc5)n4)cc(-c4nc(-c5ccccc5)nc(-c5ccccc5)n4)c3)cc21]

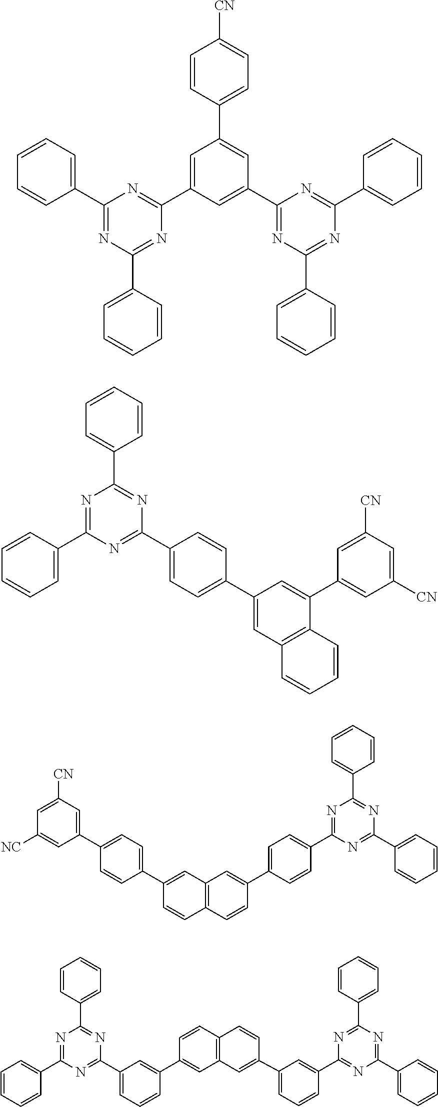 [SMILES: N#Cc1ccc(-c2cc(-c3nc(-c4ccccc4)nc(-c4ccccc4)n3)cc(-c3nc(-c4ccccc4)nc(-c4ccccc4)n3)c2)cc1 ; N#Cc1cc(C#N)cc(-c2cc(-c3ccc(-c4nc(-c5ccccc5)nc(-c5ccccc5)n4)cc3)cc3ccccc23)c1 ; N#Cc1cc(C#N)cc(-c2ccc(-c3ccc4ccc(-c5ccc(-c6nc(-c7ccccc7)nc(-c7ccccc7)n6)cc5)cc4c3)cc2)c1 ; c1ccc(-c2nc(-c3ccccc3)nc(-c3cccc(-c4ccc5ccc(-c6cccc(-c7nc(-c8ccccc8)nc(-c8ccccc8)n7)c6)cc5c4)c3)n2)cc1]

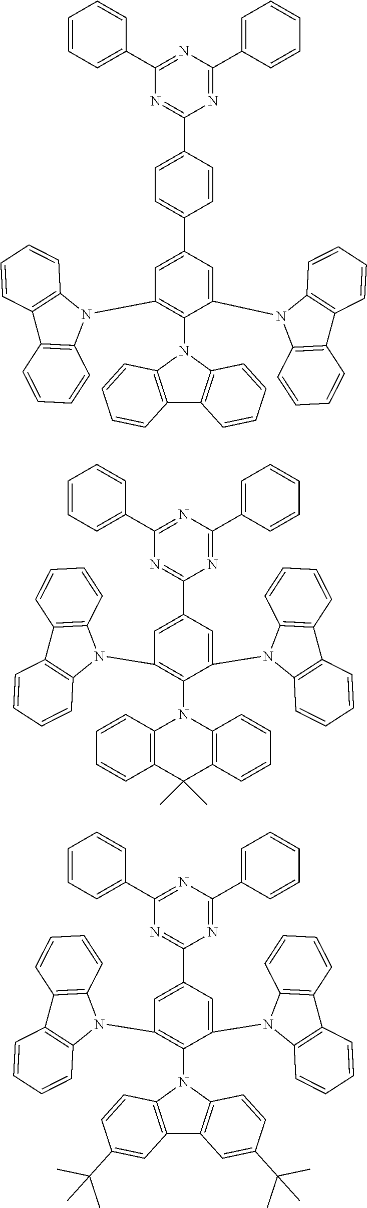

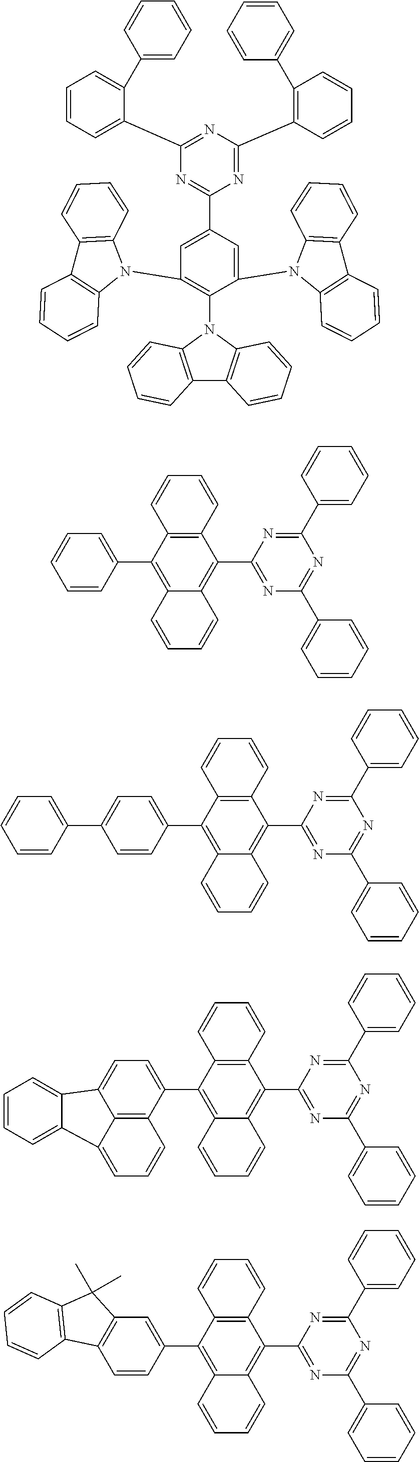

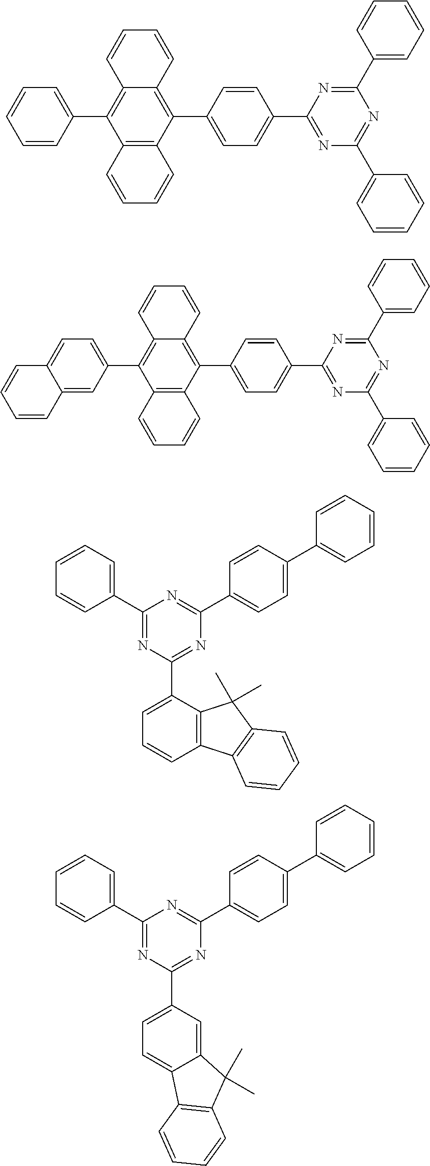

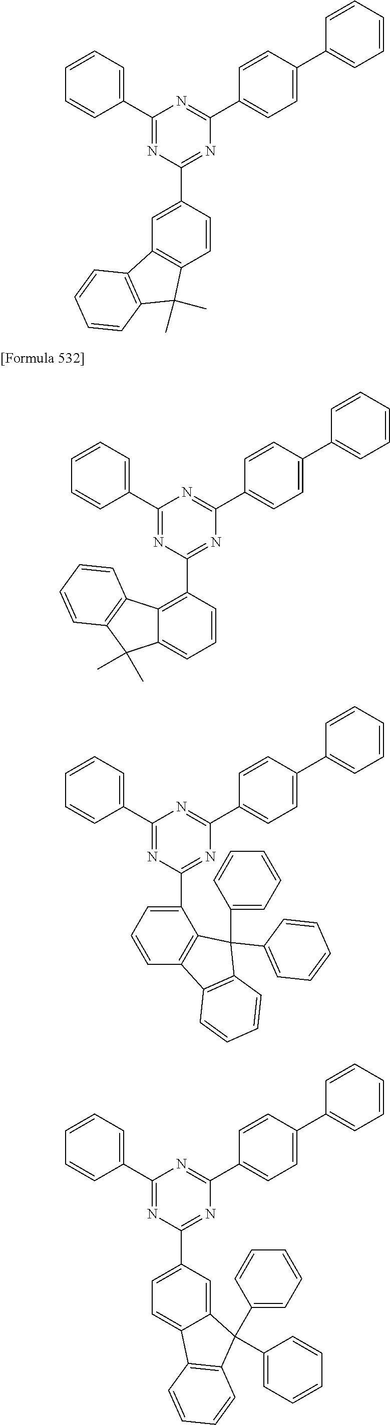 [SMILES: CC1(C)c2ccccc2-c2cc(-c3nc(-c4ccccc4)nc(-c4ccc(-c5ccccc5)cc4)n3)ccc21 ; CC1(C)c2ccccc2-c2c(-c3nc(-c4ccccc4)nc(-c4ccc(-c5ccccc5)cc4)n3)cccc21 ; c1ccc(-c2ccc(-c3nc(-c4ccccc4)nc(-c4cccc5c4C(c4ccccc4)(c4ccccc4)c4ccccc4-5)n3)cc2)cc1 ; c1ccc(-c2ccc(-c3nc(-c4ccccc4)nc(-c4ccc5c(c4)C(c4ccccc4)(c4ccccc4)c4ccccc4-5)n3)cc2)cc1]

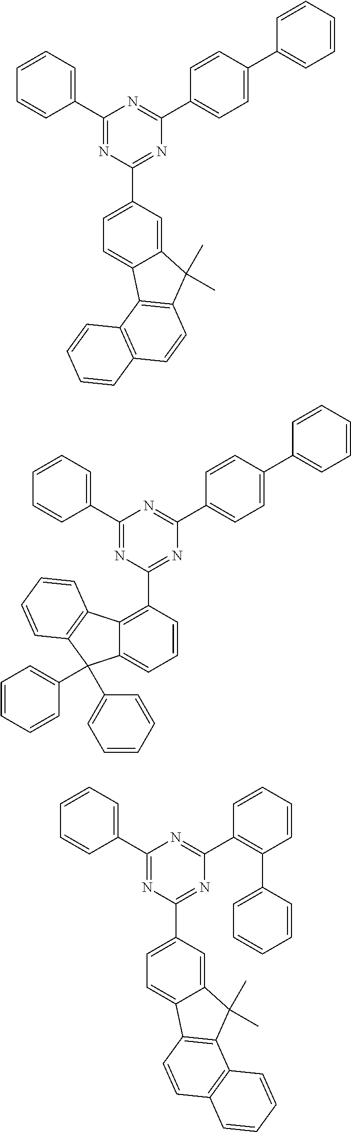

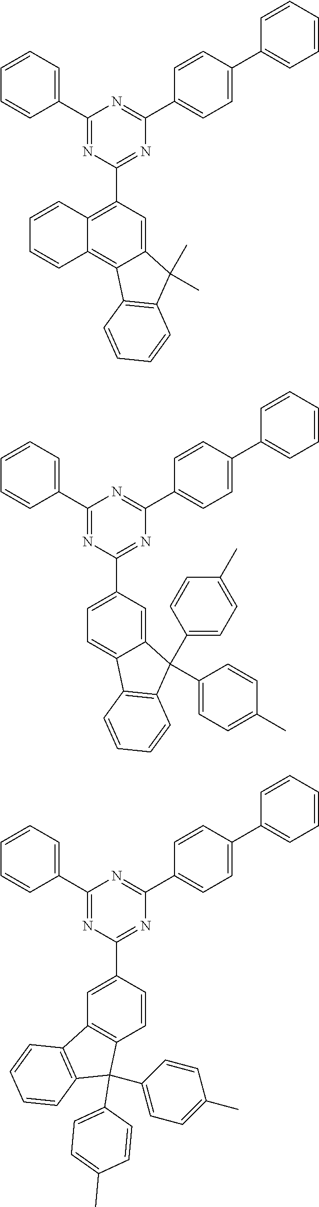

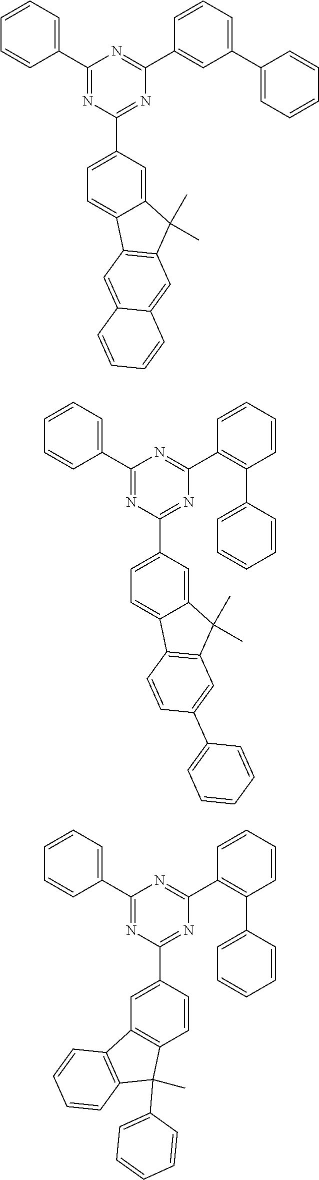

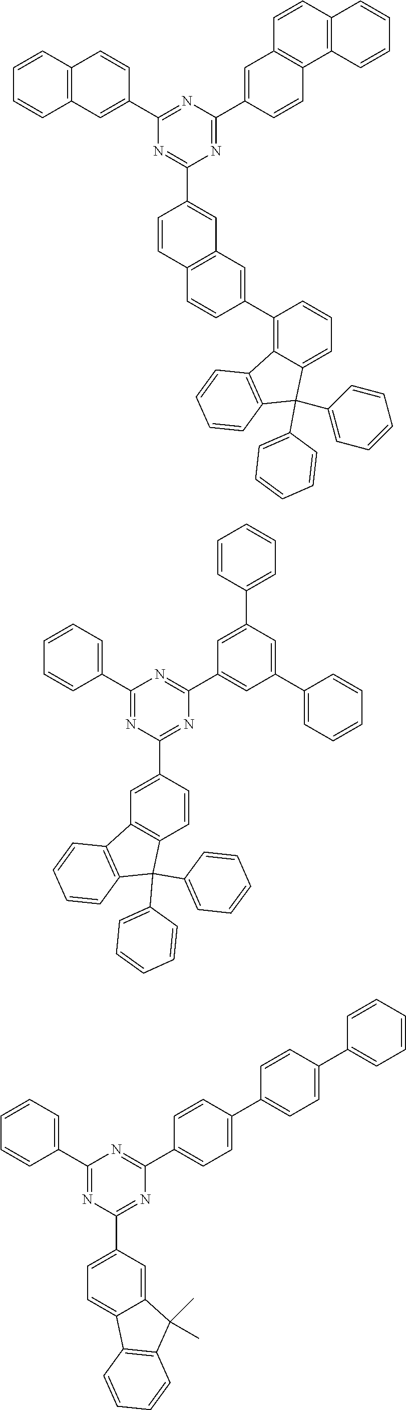

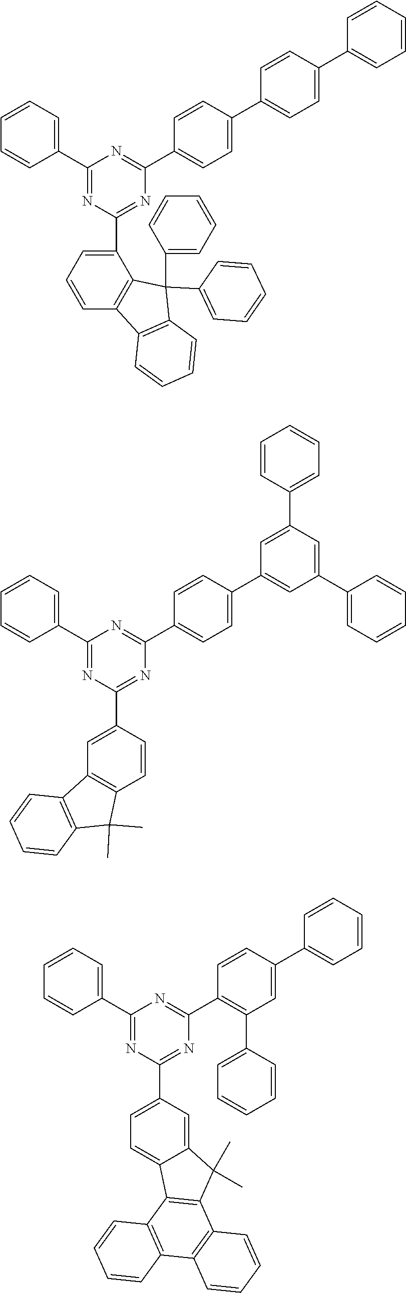 [SMILES: c1ccc(-c2ccc(-c3ccc(-c4nc(-c5ccccc5)nc(-c5cccc6c5C(c5ccccc5)(c5ccccc5)c5ccccc5-6)n4)cc3)cc2)cc1 ; CC1(C)c2ccccc2-c2cc(-c3nc(-c4ccccc4)nc(-c4ccc(-c5cc(-c6ccccc6)cc(-c6ccccc6)c5)cc4)n3)ccc21 ; CC1(C)c2cc(-c3nc(-c4ccccc4)nc(-c4ccc(-c5ccccc5)cc4-c4ccccc4)n3)ccc2-c2c1c1ccccc1c1ccccc21]

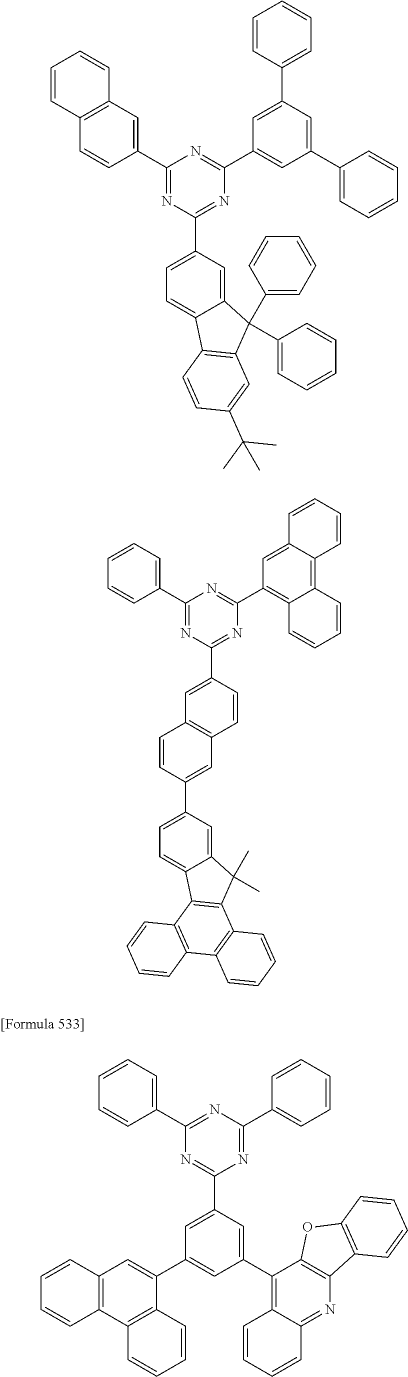 [SMILES: CC(C)(C)c1ccc2c(c1)C(c1ccccc1)(c1ccccc1)c1cc(-c3nc(-c4cc(-c5ccccc5)cc(-c5ccccc5)c4)nc(-c4ccc5ccccc5c4)n3)ccc1-2 ; CC1(C)c2cc(-c3ccc4cc(-c5nc(-c6ccccc6)nc(-c6cc7ccccc7c7ccccc67)n5)ccc4c3)ccc2-c2c1c1ccccc1c1ccccc21 ; c1ccc(-c2nc(-c3ccccc3)nc(-c3cc(-c4cc5ccccc5c5ccccc45)cc(-c4c5ccccc5nc5c4oc4ccccc45)c3)n2)cc1]

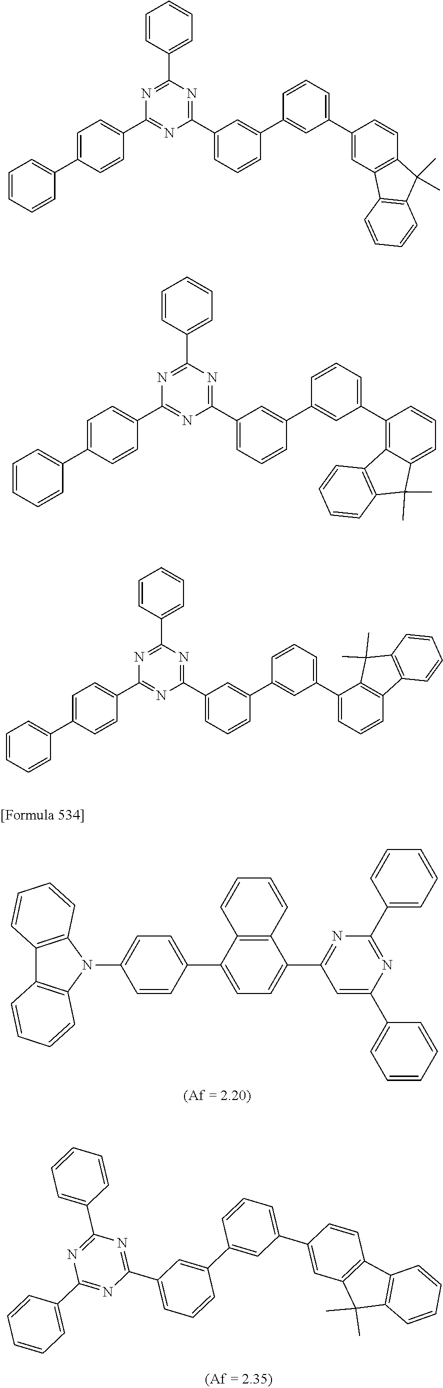 [SMILES: CC1(C)c2ccccc2-c2cc(-c3cccc(-c4cccc(-c5nc(-c6ccccc6)nc(-c6ccc(-c7ccccc7)cc6)n5)c4)c3)ccc21 ; CC1(C)c2ccccc2-c2c(-c3cccc(-c4cccc(-c5nc(-c6ccccc6)nc(-c6ccc(-c7ccccc7)cc6)n5)c4)c3)cccc21 ; CC1(C)c2ccccc2-c2cccc(-c3cccc(-c4cccc(-c5nc(-c6ccccc6)nc(-c6ccc(-c7ccccc7)cc6)n5)c4)c3)c21 ; c1ccc(-c2cc(-c3ccc(-c4ccc(-n5c6ccccc6c6ccccc65)cc4)c4ccccc34)nc(-c3ccccc3)n2)cc1 ; CC1(C)c2ccccc2-c2ccc(-c3cccc(-c4cccc(-c5nc(-c6ccccc6)nc(-c6ccccc6)n5)c4)c3)cc21]

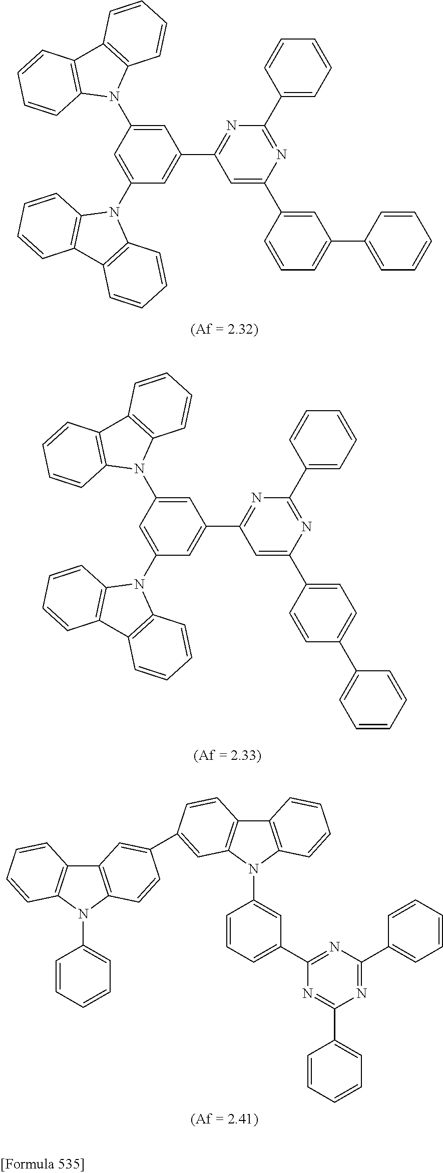 [SMILES: c1ccc(-c2cccc(-c3cc(-c4cc(-n5c6ccccc6c6ccccc65)cc(-n5c6ccccc6c6ccccc65)c4)nc(-c4ccccc4)n3)c2)cc1 ; c1ccc(-c2ccc(-c3cc(-c4cc(-n5c6ccccc6c6ccccc65)cc(-n5c6ccccc6c6ccccc65)c4)nc(-c4ccccc4)n3)cc2)cc1 ; c1ccc(-c2nc(-c3ccccc3)nc(-c3cccc(-n4c5ccccc5c5ccc(-c6ccc7c(c6)c6ccccc6n7-c6ccccc6)cc54)c3)n2)cc1]

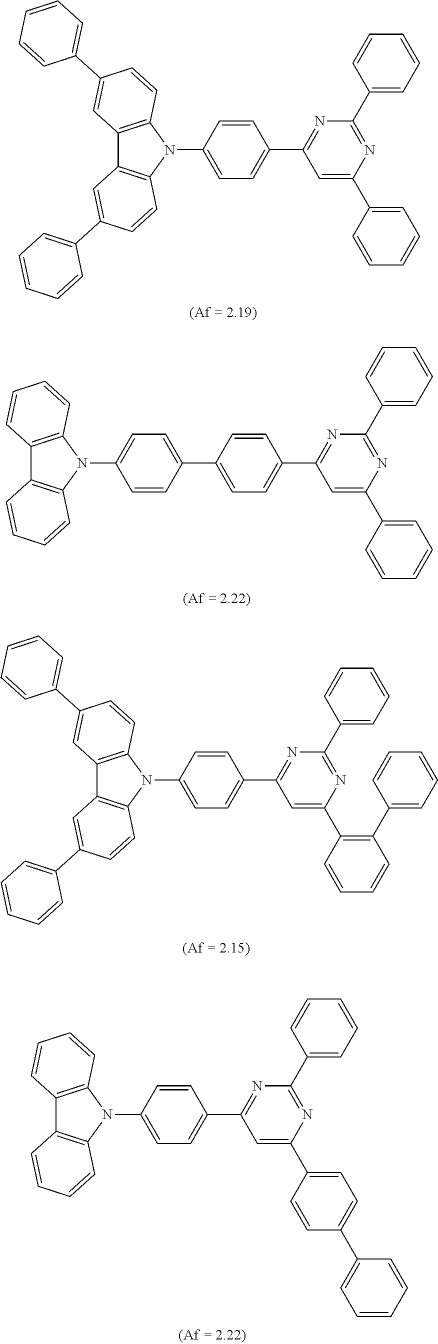 [SMILES: c1ccc(-c2ccc3c(c2)c2cc(-c4ccccc4)ccc2n3-c2ccc(-c3cc(-c4ccccc4)nc(-c4ccccc4)n3)cc2)cc1 ; c1ccc(-c2cc(-c3ccc(-c4ccc(-n5c6ccccc6c6ccccc65)cc4)cc3)nc(-c3ccccc3)n2)cc1 ; c1ccc(-c2ccc3c(c2)c2cc(-c4ccccc4)ccc2n3-c2ccc(-c3cc(-c4ccccc4-c4ccccc4)nc(-c4ccccc4)n3)cc2)cc1 ; c1ccc(-c2ccc(-c3cc(-c4ccc(-n5c6ccccc6c6ccccc65)cc4)nc(-c4ccccc4)n3)cc2)cc1]

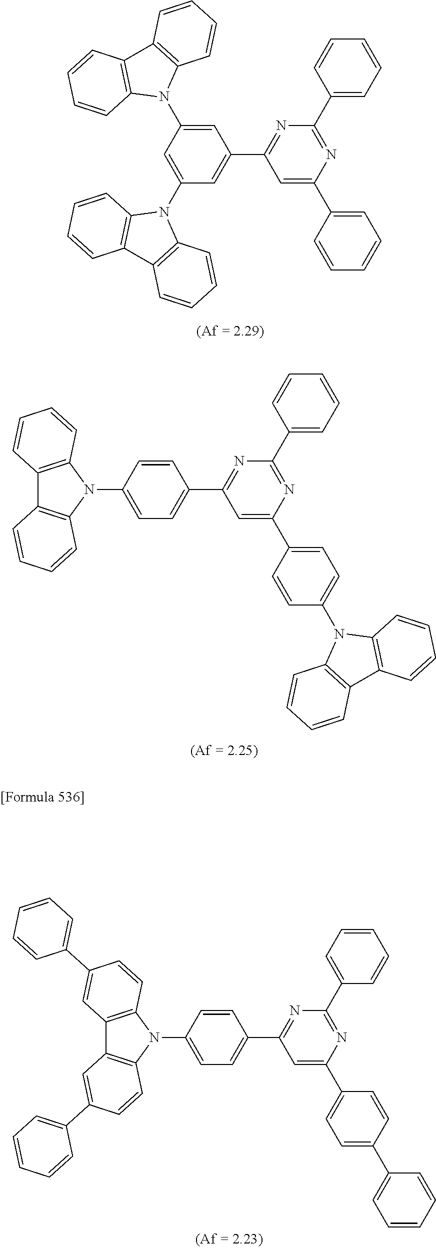 [SMILES: c1ccc(-c2cc(-c3cc(-n4c5ccccc5c5ccccc54)cc(-n4c5ccccc5c5ccccc54)c3)nc(-c3ccccc3)n2)cc1 ; c1ccc(-c2nc(-c3ccc(-n4c5ccccc5c5ccccc54)cc3)cc(-c3ccc(-n4c5ccccc5c5ccccc54)cc3)n2)cc1 ; c1ccc(-c2ccc(-c3cc(-c4ccc(-n5c6ccc(-c7ccccc7)cc6c6cc(-c7ccccc7)ccc65)cc4)nc(-c4ccccc4)n3)cc2)cc1]

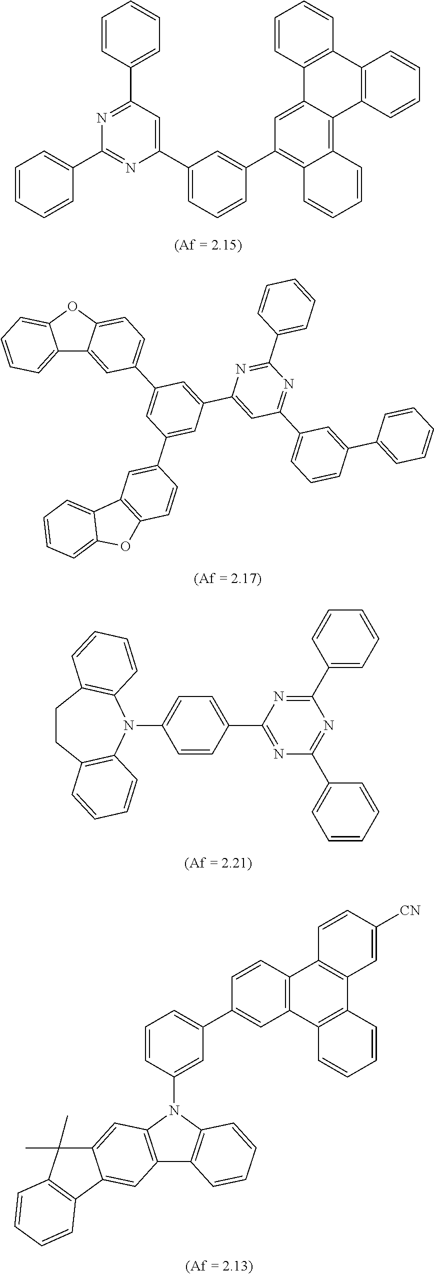 [SMILES: c1ccc(-c2cc(-c3cccc(-c4cc5c6ccccc6c6ccccc6c5c5ccccc45)c3)nc(-c3ccccc3)n2)cc1 ; c1ccc(-c2cccc(-c3cc(-c4cc(-c5ccc6oc7ccccc7c6c5)cc(-c5ccc6oc7ccccc7c6c5)c4)nc(-c4ccccc4)n3)c2)cc1 ; c1ccc(-c2nc(-c3ccccc3)nc(-c3ccc(N4c5ccccc5CCc5ccccc54)cc3)n2)cc1 ; CC1(C)c2ccccc2-c2cc3c4ccccc4n(-c4cccc(-c5ccc6c7ccc(C#N)cc7c7ccccc7c6c5)c4)c3cc21]

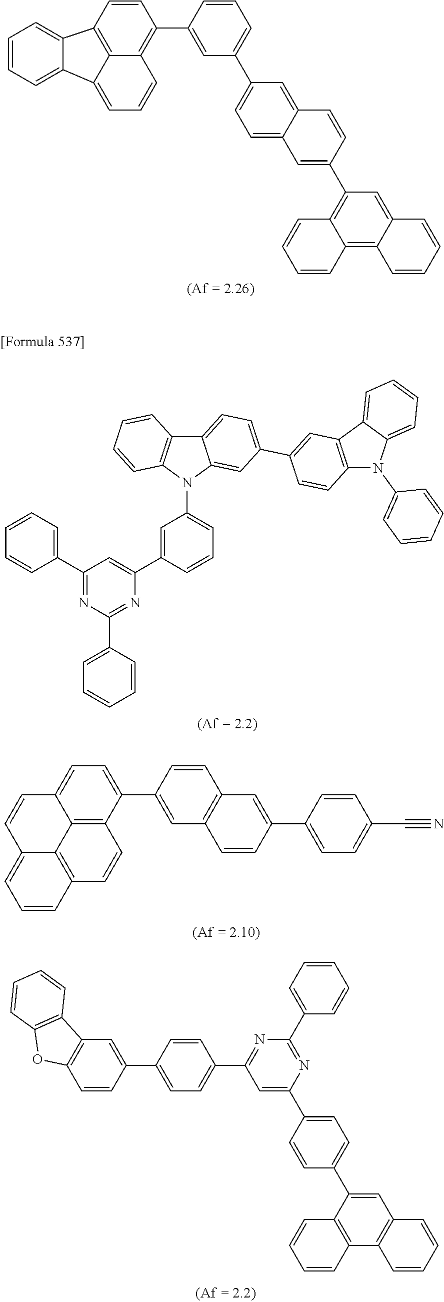

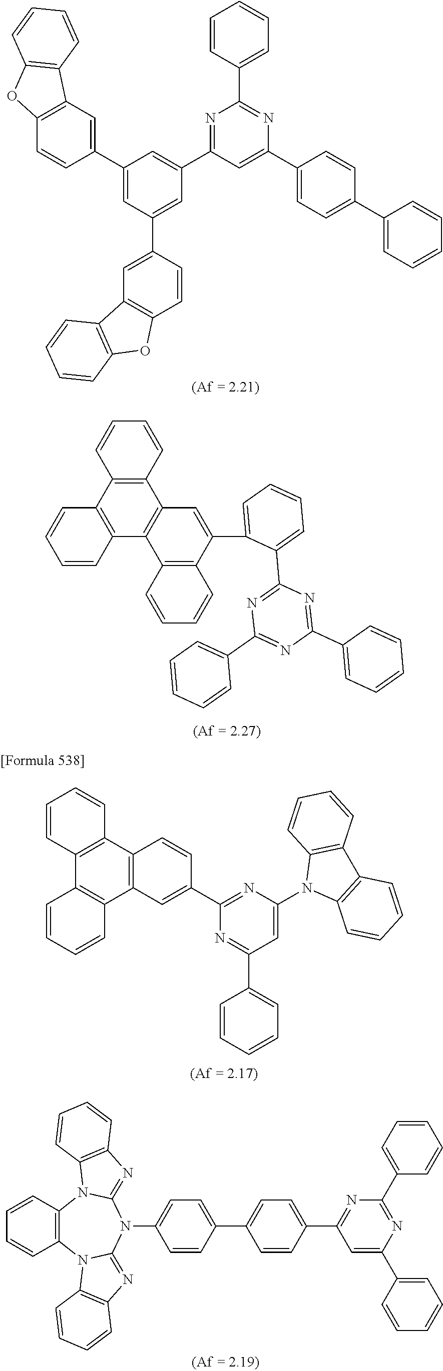

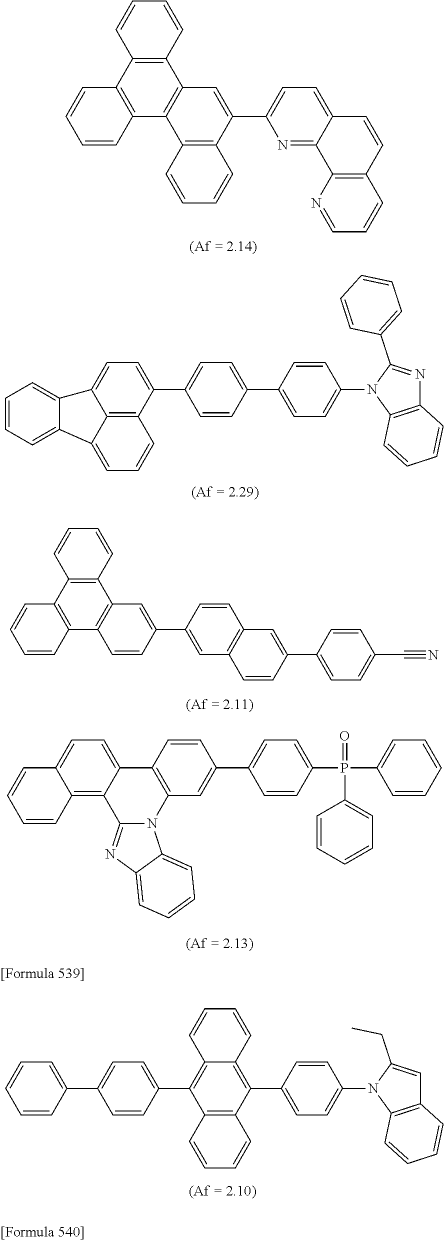 [SMILES: c1cnc2c(c1)ccc1ccc(-c3cc4c5ccccc5c5ccccc5c4c4ccccc34)nc12 ; c1ccc(-c2nc3ccccc3n2-c2ccc(-c3ccc(-c4ccc5c6c(cccc46)-c4ccccc4-5)cc3)cc2)cc1 ; N#Cc1ccc(-c2ccc3cc(-c4ccc5c6ccccc6c6ccccc6c5c4)ccc3c2)cc1 ; O=P(c1ccccc1)(c1ccccc1)c1ccc(-c2ccc3c4ccc5ccccc5c4c4nc5ccccc5n4c3c2)cc1 ; CCc1cc2ccccc2n1-c1ccc(-c2c3ccccc3c(-c3ccc(-c4ccccc4)cc3)c3ccccc23)cc1]

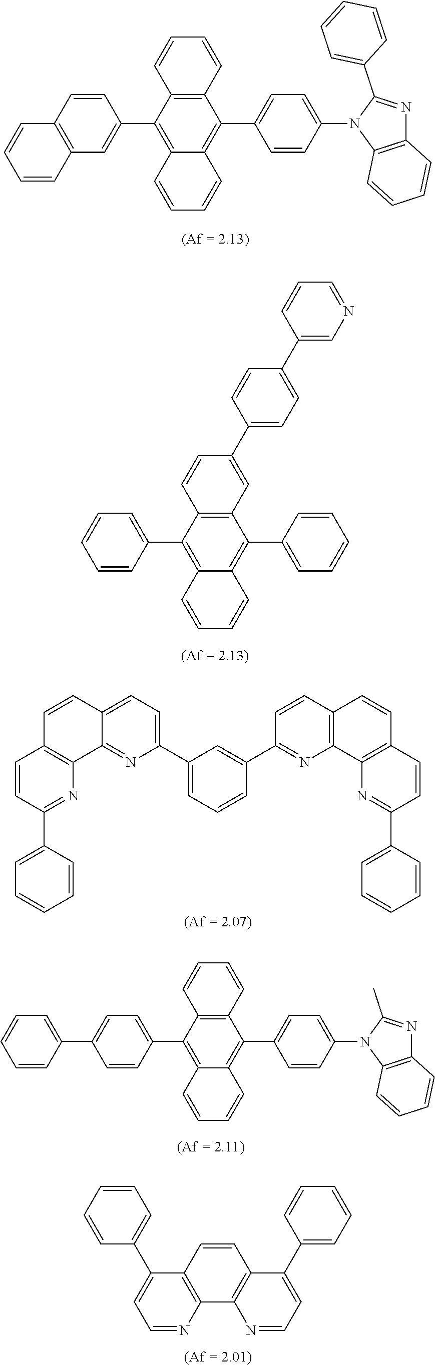 [SMILES: c1ccc(-c2nc3ccccc3n2-c2ccc(-c3c4ccccc4c(-c4ccc5ccccc5c4)c4ccccc34)cc2)cc1 ; c1ccc(-c2c3ccccc3c(-c3ccccc3)c3cc(-c4ccc(-c5cccnc5)cc4)ccc23)cc1 ; c1ccc(-c2ccc3ccc4ccc(-c5cccc(-c6ccc7ccc8ccc(-c9ccccc9)nc8c7n6)c5)nc4c3n2)cc1 ; Cc1nc2ccccc2n1-c1ccc(-c2c3ccccc3c(-c3ccc(-c4ccccc4)cc3)c3ccccc23)cc1 ; c1ccc(-c2ccnc3c2ccc2c(-c4ccccc4)ccnc23)cc1]

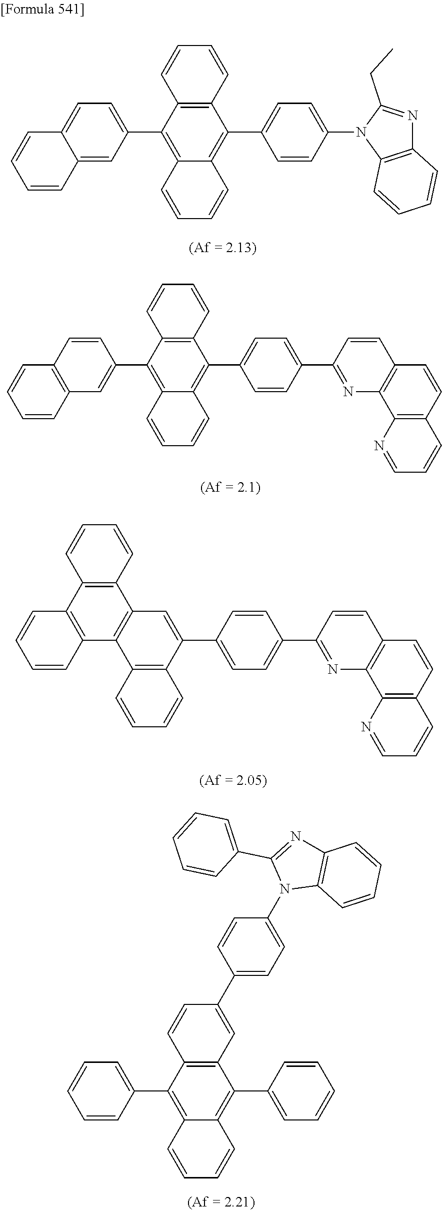 [SMILES: CCc1nc2ccccc2n1-c1ccc(-c2c3ccccc3c(-c3ccc4ccccc4c3)c3ccccc23)cc1 ; c1ccc2cc(-c3c4ccccc4c(-c4ccc(-c5ccc6ccc7cccnc7c6n5)cc4)c4ccccc34)ccc2c1 ; c1cnc2c(c1)ccc1ccc(-c3ccc(-c4cc5c6ccccc6c6ccccc6c5c5ccccc45)cc3)nc12 ; c1ccc(-c2c3ccccc3c(-c3ccccc3)c3cc(-c4ccc(-n5c(-c6ccccc6)nc6ccccc65)cc4)ccc23)cc1]

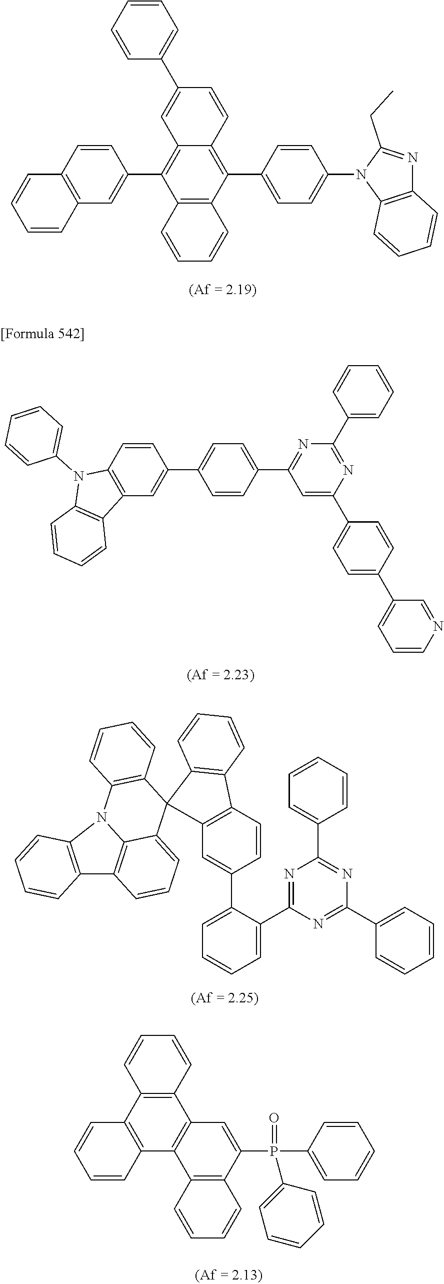 [SMILES: CCc1nc2ccccc2n1-c1ccc(-c2c3ccccc3c(-c3ccc4ccccc4c3)c3cc(-c4ccccc4)ccc23)cc1 ; c1ccc(-c2nc(-c3ccc(-c4cccnc4)cc3)cc(-c3ccc(-c4ccc5c(c4)c4ccccc4n5-c4ccccc4)cc3)n2)cc1 ; c1ccc(-c2nc(-c3ccccc3)nc(-c3ccccc3-c3ccc4c(c3)C3(c5ccccc5-4)c4ccccc4-n4c5ccccc5c5cccc3c54)n2)cc1 ; O=P(c1ccccc1)(c1ccccc1)c1cc2c3ccccc3c3ccccc3c2c2ccccc12]

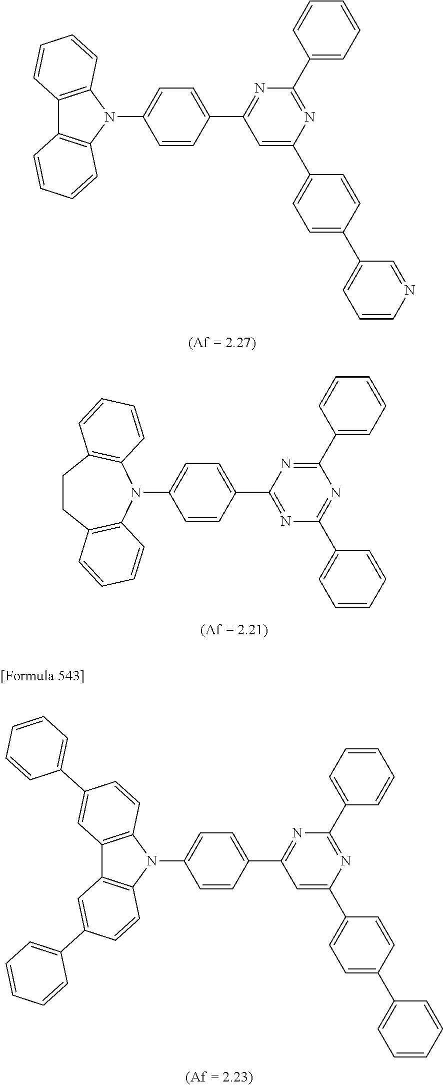 [SMILES: c1ccc(-c2nc(-c3ccc(-c4cccnc4)cc3)cc(-c3ccc(-n4c5ccccc5c5ccccc54)cc3)n2)cc1 ; c1ccc(-c2nc(-c3ccccc3)nc(-c3ccc(N4c5ccccc5CCc5ccccc54)cc3)n2)cc1 ; c1ccc(-c2ccc(-c3cc(-c4ccc(-n5c6ccc(-c7ccccc7)cc6c6cc(-c7ccccc7)ccc65)cc4)nc(-c4ccccc4)n3)cc2)cc1]

A second exemplary embodiment and a third exemplary embodiment of the invention will be described below.

An organic EL device according to each of the second and third exemplary embodiments is one arrangement of the organic EL device according to the first exemplary embodiment.

Thus, elements that may be included in the organic EL device of the second exemplary embodiment are similar to the elements that may be included in the organic EL device described in the first exemplary embodiment. Likewise, elements that may be included in the organic EL device of the third exemplary embodiment are similar to the elements that may be included in the organic EL device described in the first exemplary embodiment.

### Second Exemplary Embodiment

The organic EL device according to the second exemplary embodiment includes: a cathode; an anode; an emitting region disposed between the cathode and the anode; a hole transporting zone disposed between the anode and the emitting region, and an electron transporting zone disposed between the cathode and the emitting region, in which the emitting region includes one or more emitting layers, the one or more emitting layers include a first emitting layer, the first emitting layer contains a first host material, a first additional host material different from the first host material, and a first luminescent compound, the electron transporting zone includes at least one electron transporting layer, the hole transporting zone includes a first anode side organic layer and a second anode side organic layer, the first anode side organic layer is in direct contact with the second anode side organic layer, the first anode side organic layer and the second anode side organic layer are disposed between the anode and the emitting region in this order from a side close to the anode, the first anode side organic layer contains a first organic material and a second organic material, the first organic material is different from the second organic material, a content of the first organic material in the first anode side organic layer is less than 50 mass %, the second organic material is a monoamine compound having one substituted or unsubstituted amino group in a molecule, or a diamine compound having two substituted or unsubstituted amino groups in a molecule, the second anode side organic layer contains a second hole transporting zone material, the second hole transporting zone material is a monoamine compound having one substituted or unsubstituted amino group in a molecule, or a diamine compound having two substituted or unsubstituted amino groups in a molecule, the second hole transporting zone material is different from the first organic material, the second hole transporting zone material and the second organic material are mutually the same or different, the at least one electron transporting layer in the electron transporting zone contains a phenanthroline compound having a phenanthroline skeleton, the phenanthroline compound is a compound that has at least one group represented by the formula (21) and is represented by the formula (20), the first emitting layer is an emitting layer that emits fluorescence, and a refractive index NM₁ of the constituent materials contained in the first anode side organic layer and a refractive index NM₂ of the constituent material contained in the second anode side organic layer satisfy a relationship of a numerical formula below (Numerical Formula NM).

$${NM}_{1} > {NM}_{2} \qquad ( Numerical Formula NM )$$

According to the second exemplary embodiment, the organic EL device has improved device performance. In an exemplary arrangement according to the second exemplary embodiment, the organic EL device has improved luminous efficiency. In an exemplary arrangement according to the second exemplary embodiment, the organic EL device has a longer lifetime.

### Third Exemplary Embodiment

The organic EL device according to the third exemplary embodiment includes: a cathode; an anode; an emitting region disposed between the cathode and the anode; a hole transporting zone disposed between the anode and the emitting region, in which the emitting region includes one or more emitting layers, the one or more emitting layers include a first emitting layer, the first emitting layer contains a first host material, a first additional host material different from the first host material, and a first luminescent compound, the first host material is a compound represented by the formula (H10), the first additional host material is a compound represented by the formula (H20), the compound represented by the formula (H20) has at least one deuterium atom, the hole transporting zone includes a first anode side organic layer and a second anode side organic layer, the first anode side organic layer is in direct contact with the second anode side organic layer, the first anode side organic layer and the second anode side organic layer are disposed between the anode and the emitting region in this order from a side close to the anode, the first anode side organic layer contains a first organic material and a second organic material, the first organic material is different from the second organic material, a content of the first organic material in the first anode side organic layer is less than 50 mass %, the second organic material is a monoamine compound having one substituted or unsubstituted amino group in a molecule, or a diamine compound having two substituted or unsubstituted amino groups in a molecule, the second anode side organic layer contains a second hole transporting zone material, the second hole transporting zone material is a monoamine compound having one substituted or unsubstituted amino group in a molecule, or a diamine compound having two substituted or unsubstituted amino groups in a molecule, the second hole transporting zone material is different from the first organic material, the second hole transporting zone material and the second organic material are mutually the same or different, the first emitting layer is an emitting layer that emits fluorescence, and a refractive index NM₁ of the constituent materials contained in the first anode side organic layer and a refractive index NM₂ of the constituent material contained in the second anode side organic layer satisfy a relationship of a numerical formula below (Numerical Formula NM).

$${NM}_{1} > {NM}_{2} \qquad ( Numerical Formula NM )$$

According to the third exemplary embodiment, the organic EL device has improved device performance. In an exemplary arrangement according to the third exemplary embodiment, the organic EL device has improved luminous efficiency. In an exemplary arrangement according to the third exemplary embodiment, the organic EL device has a longer lifetime.

### Additional Layers of Organic EL Device

The organic EL devices according to the first to third exemplary embodiments may each further include one or more organic layers in addition to the first anode side organic layer, the second anode side organic layer and the first emitting layer. Those organic layers include, for instance, at least one layer selected from the group consisting of an electron injecting layer, a hole blocking layer and an electron blocking layer, in addition to the third anode side organic layer, the fourth anode side organic layer, the second emitting layer and the electron transporting layer.

In the organic EL devices according to the first to third exemplary embodiments, the arrangement of the organic layers may consist of the first anode side organic layer, the second anode side organic layer, and the first emitting layer. Alternatively, the arrangement of the organic layers may further include, for instance, at least one layer selected from the group consisting of the third anode side organic Layer, the fourth anode side organic layer, the second emitting layer, the electron injecting layer, the electron transporting layer, the hole blocking layer and other layers.

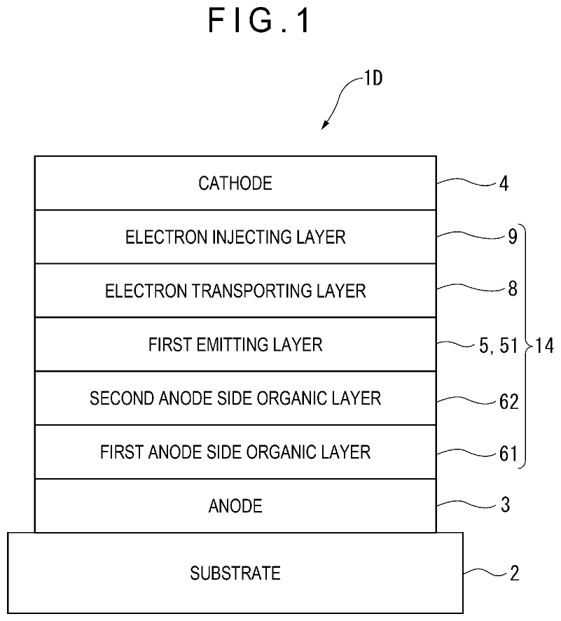

FIG. 1 schematically depicts an exemplary arrangement of an organic EL device according to the first exemplary embodiment.

An organic EL device 1D includes a substrate 2, an anode 3, a cathode 4, and organic layers 14 disposed between the anode 3 and the cathode 4. The organic layers 14 include a first anode side organic layer 61, a second anode side organic layer 62, a first emitting layer 51, an electron transporting layer 8, and an electron injecting layer 9, which are layered in this order from a side close to the anode 3.

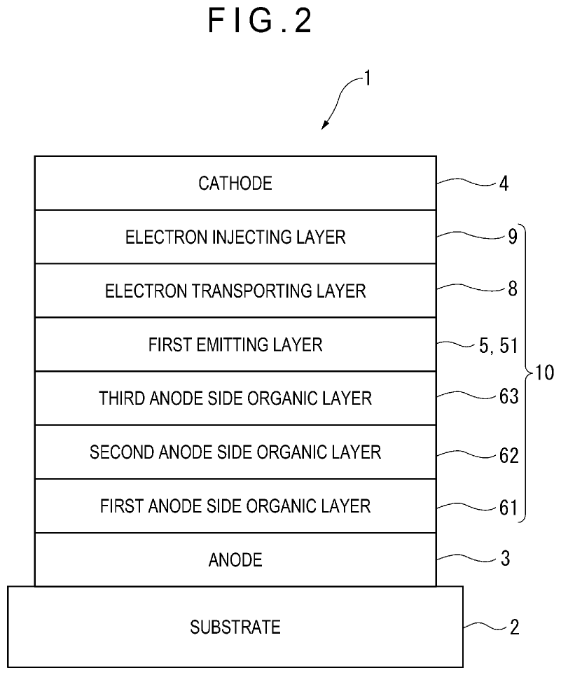

FIG. 2 schematically depicts another exemplary arrangement of an organic EL device according to the first exemplary embodiment.

An organic EL device 1 includes a substrate 2, an anode 3, a cathode 4, and organic layers 10 disposed between the anode 3 and the cathode 4. The organic layers 10 include a first anode side organic layer 61, a second anode side organic layer 62, a third anode side organic layer 63, a first emitting layer 51, an electron transporting layer 8, and an electron injecting layer 9, which are layered in this order from a side close to the anode 3.

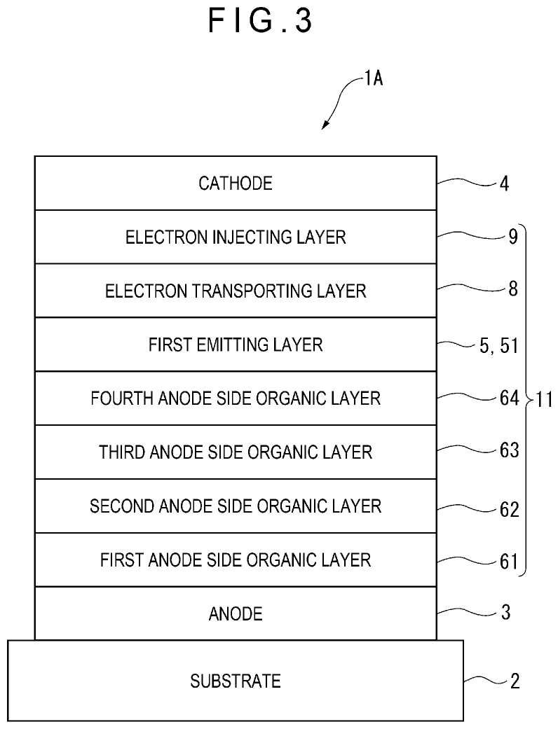

FIG. 3 schematically depicts a still another exemplary arrangement of the organic EL device according to the first exemplary embodiment.

An organic EL device 1A includes a substrate 2, an anode 3, a cathode 4, and organic layers 11 disposed between the anode 3 and the cathode 4. The organic layers 11 include a first anode side organic layer 61, a second anode side organic layer 62, a third anode side organic layer 63, a fourth anode side organic layer 64, a first emitting layer 51, an electron transporting layer 8, and an electron injecting layer 9, which are layered in this order from a side close to the anode 3.

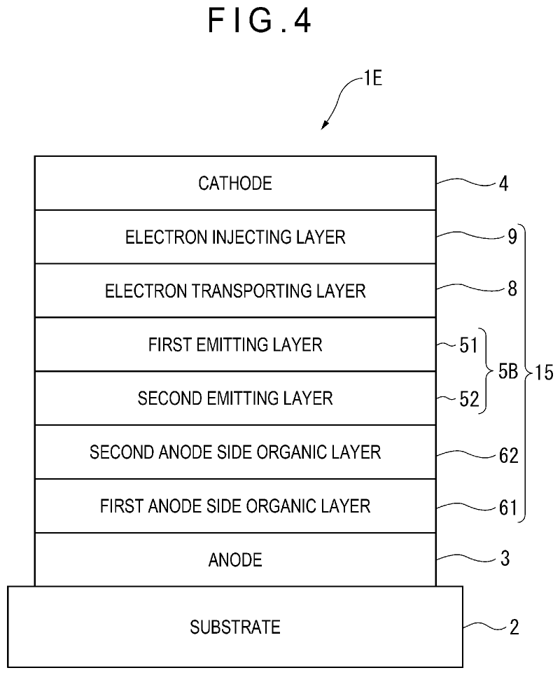

FIG. 4 schematically depicts a further exemplary arrangement of the organic EL device according to the first exemplary embodiment.

An organic EL device 1E includes a substrate 2, an anode 3, a cathode 4, and organic layers 15 disposed between the anode 3 and the cathode 4. The organic layers 15 include a first anode side organic layer 61, a second anode side organic layer 62, a second emitting layer 52, a first emitting layer 51, an electron transporting layer 8, and an electron injecting layer 9, which are layered in this order from a side close to the anode 3.

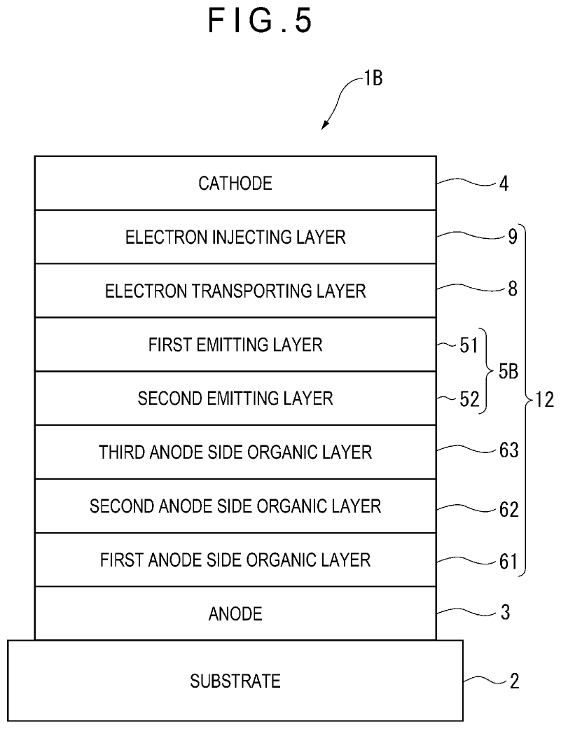

FIG. 5 schematically depicts a still further exemplary arrangement of the organic EL device according to the first exemplary embodiment.

An organic EL device 1B includes a substrate 2, an anode 3, a cathode 4, and organic layers 12 disposed between the anode 3 and the cathode 4. The organic layers 12 include a first anode side organic layer 61, a second anode side organic layer 62, a third anode side organic layer 63, a second emitting layer 52, a first emitting layer 51, an electron transporting layer 8, and an electron injecting layer 9, which are layered in this order from a side close to the anode 3.

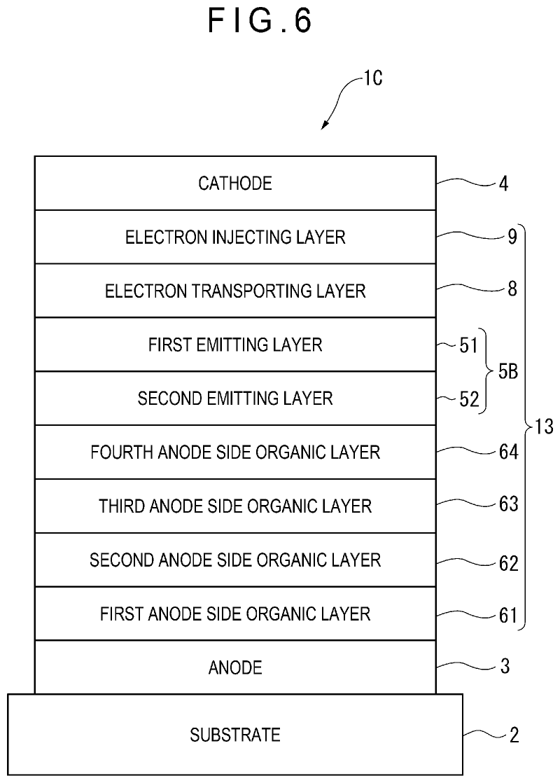

FIG. 6 schematically depicts a still further exemplary arrangement of the organic EL device according to the first exemplary embodiment.

An organic EL device 1C includes a substrate 2, an anode 3, a cathode 4, and organic layers 13 disposed between the anode 3 and the cathode 4. The organic layers 13 include a first anode side organic layer 61, a second anode side organic layer 62, a third anode side organic layer 63, a fourth anode side organic layer 64, a second emitting layer 52, a first emitting layer 51, an electron transporting layer 8, and an electron injecting layer 9, which are layered in this order from a side close to the anode 3.

In the organic EL device 1D of FIG. 1, the organic EL device 1 of FIG. 2, and the organic EL device 1A of FIG. 3, the emitting region 5 includes the first emitting layer 51.

In the organic EL device 1E of FIG. 4, the organic EL device 1B of FIG. 5, and the organic EL device 1C of FIG. 6, the emitting region 5B includes the first emitting layer 51 and the second emitting layer 52.

In the organic EL device 1D of FIG. 1 and the organic EL device 1E of FIG. 4, the hole transporting zone includes the first anode side organic layer 61 and the second anode side organic layer 62.

In the organic EL device 1 of FIG. 2 and the organic EL device 1B of FIG. 5, the hole transporting zone includes the first anode side organic layer 61, the second anode side organic layer 62, and the third anode side organic layer 63.

In the organic EL device 1A in FIG. 3 and the organic EL device 1C in FIG. 6, the hole transporting zone includes the first anode side organic layer 61, the second anode side organic layer 62, the third anode side organic layer 63, and the fourth anode side organic layer 64.

The arrangements of the organic EL devices depicted in FIGS. 1 to 6 are also applicable to the organic EL device according to the second or third exemplary embodiment.

The invention is not limited to the exemplary arrangements of the organic EL devices depicted in FIGS. 1 to 6. Examples of a further arrangement of the organic EL device include an arrangement of the organic EL device in which the first emitting layer and the second emitting layer in the emitting region are layered in this order from a side close to the anode.

### Interposed Layer

In an exemplary arrangement of the organic EL device according to the exemplary embodiment, when the emitting region includes the first emitting layer and the second emitting layer, an interposed layer may be included as an organic layer disposed between the first emitting layer and the second emitting layer.

In an exemplary embodiment, in order to inhibit an overlap between a Singlet emitting region and a TTF emitting region, the interposed layer contains no luminescent compound or may contain a luminescent compound in an insubstantial amount provided that the overlap can be inhibited.

For instance, the interposed layer contains 0 mass % of a luminescent compound. Alternatively, for instance, the interposed layer may contain a luminescent compound provided that the luminescent compound contained is a component accidentally mixed in a producing process or a component contained as impurities in a material.

For instance, when the interposed layer consists of a material A, a material B, and a material C, the content ratios of the materials A, B, and C in the interposed layer are each 10 mass % or more, and the total of the content ratios of the materials A, B, and C is 100 mass %.

In the following, the interposed layer is occasionally referred to as a “non-doped layer”. A layer containing a luminescent compound is occasionally referred to as a “doped layer”.

It is considered that luminous efficiency is improvable in an arrangement including layered emitting layers because the Singlet emitting region and the TTF emitting region are typically likely to be separated from each other.

In an exemplary arrangement of the organic EL device according to the exemplary embodiment, when the interposed layer (non-doped layer) is disposed between the first emitting layer and the second emitting layer in the emitting region, it is expected that a region where the Singlet emitting region and the TTF emitting region overlap with each other is reduced to inhibit a decrease in TTF efficiency caused by collision between triplet excitons and carriers. That is, it is considered that providing the interposed layer (non-doped layer) between the emitting layers contributes to the improvement in the efficiency of TTF emission.

The interposed layer is a non-doped layer.

The interposed layer contains no metal atom. The interposed layer thus contains no metal complex.

The interposed layer contains an interposed layer material. The interposed layer material is not a luminescent compound.

The interposed layer material may be any material except for the luminescent compound.

Examples of the interposed layer material include: 1) a heterocyclic compound such as an oxadiazole derivative, benzimidazole derivative, or phenanthroline derivative; 2) a fused aromatic compound such as a carbazole derivative, anthracene derivative, phenanthrene derivative, pyrene derivative or chrysene derivative; and 3) an aromatic amine compound such as a triarylamine derivative or a fused polycyclic aromatic amine derivative.

One or both of the first host material and the second host material may be used as the interposed layer material. The interposed layer material may be any material provided that the Singlet emitting region and the TTF emitting region are separated from each other and the Singlet emission and the TTF emission are not hindered.

In an exemplary arrangement of the organic EL device according to the exemplary embodiment, the content ratios of all the materials forming the interposed layer in the interposed layer are each 10 mass % or more.

The interposed layer contains the interposed layer material as a material forming the interposed layer.

The interposed layer preferably contains 60 mass % or more of the interposed layer material, more preferably contains 70 mass % or more of the interposed layer material, still more preferably contains 80 mass % or more of the interposed layer material, still further more preferably 90 mass % or more of the interposed layer material, and yet still further more preferably 95 mass % or more of the interposed layer material, with respect to the total mass of the interposed layer.

The interposed layer may contain a single type of the interposed layer material or may contain two or more types of the interposed layer material.

When the interposed layer contains two or more types of the interposed layer material, an upper limit of the total of the content ratios of the two or more types of the interposed layer material is 100 mass %.

It should be noted that the interposed layer of the exemplary embodiment may further contain any other material than the interposed layer material.

The interposed layer may be provided in the form of a single layer or a laminate of two or more layers.

As long as the overlap between the Singlet emitting region and the TTF emitting region is inhibited, a film thickness of the interposed layer is not particularly limited but each layer in the interposed layer is preferably in a range from 3 nm to 15 nm, more preferably in a range from 5 nm to 10 nm.

The interposed layer having a film thickness of 3 nm or more easily separates the Singlet emitting region from the emitting region derived from TTF.

The interposed layer having a film thickness of 15 nm or less easily inhibits a phenomenon where the host material of the interposed layer emits light.

It is preferable that the interposed layer contains the interposed layer material as a material forming the interposed layer and the triplet energy of the first host material T₁(H1), the triplet energy of the second host material T₁(H2), and a triplet energy of at least one interposed layer material T₁(M_{mid}) satisfy a relationship of a numerical formula (Numerical Formula 21) below.

$${T}_{1} ( H 2 ) \geq {T}_{1} ( {M}_{mid} ) \geq {T}_{1} ( H 1 ) \qquad ( Numerical Formula 21 )$$

When the interposed layer contains two or more interposed layer materials as a material forming the interposed layer, the triplet energy of the first host material T₁(H1), the triplet energy of the second host material T₁(H2), and a triplet energy of each interposed layer material T₁(MEA) more preferably satisfy a relationship of a numerical formula (Numerical Formula 21A) below.

$${T}_{1} ( H 2 ) \geq {T}_{1} ( {M}_{EA} ) \geq {T}_{1} ( H 1 ) \qquad ( Numerical Formula 21 A )$$

An exemplary arrangement of the organic EL device according to the exemplary embodiment may further include a diffusion layer.

When an exemplary arrangement of the organic EL device according to the exemplary embodiment includes the diffusion layer, the diffusion layer is preferably disposed between the first emitting layer and the second emitting layer.

The arrangement of the organic EL device will be further described below. It should be noted that the reference numerals are occasionally omitted below.

### Substrate

The substrate is used as a support for the organic EL device. For instance, glass, quartz, plastics and the like are usable for the substrate. A flexible substrate is also usable. The flexible substrate, which is a bendable substrate, is exemplified by a plastic substrate. Examples of a material for the flexible substrate include polycarbonate, polyarylate, polyethersulfone, polypropylene, polyester, polyvinyl fluoride, polyvinyl chloride, polyimide, and polyethylene naphthalate. Further, an inorganic vapor deposition film is also usable.

### Anode

Metal, an alloy, an electrically conductive compound, a mixture thereof, or the like having a large work function (specifically, 4.0 eV or more) is preferably used as the anode formed on the substrate. Specific examples of the material include indium tin oxide (ITO), indium oxide-tin oxide containing silicon or silicon oxide, indium oxide-zinc oxide, indium oxide containing tungsten oxide and zinc oxide, and graphene. In addition, gold (Au), platinum (Pt), nickel (Ni), tungsten (W), chrome (Cr), molybdenum (Mo), iron (Fe), cobalt (Co), copper (Cu), palladium (Pd), titanium (Ti), and nitrides of a metal material (e.g., titanium nitride) are usable.

The material is typically formed into a film by a sputtering method. For instance, the indium oxide-zinc oxide can be formed into a film by the sputtering method using a target in which zinc oxide in a range from 1 mass % to 10 mass % is added to indium oxide. Moreover, for instance, the indium oxide containing tungsten oxide and zinc oxide can be formed by the sputtering method using a target in which tungsten oxide in a range from 0.5 mass % to 5 mass % and zinc oxide in a range from 0.1 mass % to 1 mass % are added to indium oxide. In addition, the anode may be formed by a vacuum deposition method, a coating method, an inkjet method, a spin coating method or the like.

Among the EL layers formed on the anode, since the hole injecting layer adjacent to the anode is formed of a composite material into which holes are easily injectable irrespective of the work function of the anode, a material usable as an electrode material (e.g., metal, an alloy, an electroconductive compound, a mixture thereof, and the elements belonging to the group 1 or 2 of the periodic table) is also usable for the anode.

A material having a small work function such as elements belonging to Groups 1 and 2 in the periodic table of the elements, specifically, an alkali metal such as lithium (Li) and cesium (Cs), an alkaline earth metal such as magnesium (Mg), calcium (Ca) and strontium (Sr), alloys (e.g., MgAg and AlLi) including the alkali metal or the alkaline earth metal, a rare earth metal such as europium (Eu) and ytterbium (Yb), alloys including the rare earth metal are also usable for the anode. It should be noted that the vacuum deposition method and the sputtering method are usable for forming the anode using the alkali metal, alkaline earth metal and the alloy thereof. Further, when a silver paste is used for the anode, the coating method and the inkjet method are usable.

### Cathode

It is preferable to use metal, an alloy, an electroconductive compound, a mixture thereof, or the like having a small work function (specifically, 3.8 eV or less) for the cathode. Examples of the material for the cathode include elements belonging to Groups 1 and 2 in the periodic table of the elements, specifically, an alkali metal such as lithium (Li) and cesium (Cs), an alkaline earth metal such as magnesium (Mg), calcium (Ca) and strontium (Sr), alloys (e.g., MgAg and AlLi) including the alkali metal or the alkaline earth metal, a rare earth metal such as europium (Eu) and ytterbium (Yb), and alloys including the rare earth metal.

It should be noted that the vacuum deposition method and the sputtering method are usable for forming the cathode using the alkali metal, alkaline earth metal and the alloy thereof. Further, when a silver paste is used for the cathode, the coating method and the inkjet method are usable.

By providing the electron injecting layer, various conductive materials such as Al, Ag, ITO, graphene, and indium oxide-tin oxide containing silicon or silicon oxide may be used for forming the cathode regardless of the work function. The conductive materials can be formed into a film using the sputtering method, inkjet method, spin coating method, and the like.

### Electron Transporting Layer

In an exemplary arrangement of the organic EL device of the exemplary embodiment, an electron transporting layer is disposed between the emitting region and the cathode.

The electron transporting layer is a layer containing a highly electron-transportable substance. For the electron transporting layer, 1) a metal complex such as an aluminum complex, beryllium complex, and zinc complex, 2) a hetero aromatic compound such as imidazole derivative, benzimidazole derivative, azine derivative, carbazole derivative, and phenanthroline derivative, and 3) a high polymer compound are usable. Specifically, as a low-molecule organic compound, a metal complex such as Alq, tris(4-methyl-8-quinolinolato)aluminum (abbreviation: Almq₃), bis(10-hydroxybenzo[h]quinolinato)beryllium (abbreviation: BeBq₂), BAlq, Znq, ZnPBO and ZnBTZ is usable. In addition to the metal complex, a heteroaromatic compound such as 2-(4-biphenylyl)-5-(4-tert-butylphenyl)-1,3,4-oxadiazole (abbreviation: PBD), 1,3-bis[5-(ptert-butylphenyl)-1,3,4-oxadiazole-2-yl]benzene (abbreviation: OXD-7), 3-(4-tert-butylphenyl)-4-phenyl-5-(4-biphenylyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), bathophenanthroline (abbreviation: BPhen), bathocuproine (abbreviation: BCP), and 4,4′-bis(5-methylbenzoxazole-2-yl)stilbene (abbreviation: BzOs) is usable. In an exemplary arrangement of the exemplary embodiment, the nitrogen-containing compound (preferably, a benzimidazole compound) is preferably usable. The above-described substances mostly have an electron mobility of 10⁻⁶ cm²/V·s or more. It should be noted that any other substance than the above substance may be used for the electron transporting layer as long as the substance exhibits a higher electron transportability than the hole transportability. The electron transporting layer may be a single layer or a laminate of two or more layers formed of the above substance.

Further, a high polymer compound is usable for the electron transporting layer. For instance, poly[(9,9-dihexylfluorene-2,7-diyl)-co-(pyridine-3,5-diyl)](abbreviation: PF-Py), and poly[(9,9-dioctylfluorene-2,7-diyl)-co-(2,2′-bipyridine-6,6′-diyl)] (abbreviation: PF-BPy) are usable.

### Electron Injecting Layer

The electron injecting layer is a layer containing a highly electron-injectable substance. Examples of a material for the electron injecting layer include an alkali metal, alkaline earth metal and a compound thereof, examples of which include lithium (Li), cesium (Cs), calcium (Ca), lithium fluoride (LiF), cesium fluoride (CsF), calcium fluoride (CaF₂), and lithium oxide (LiOx). In addition, the alkali metal, alkaline earth metal or the compound thereof may be added to the substance exhibiting the electron transportability in use. Specifically, for instance, magnesium (Mg) added to Alq may be used. In this case, the electrons can be more efficiently injected from the cathode.

Alternatively, the electron injecting layer may be provided by a composite material in a form of a mixture of the organic compound and the electron donor. Such a composite material exhibits excellent electron injectability and electron transportability since electrons are generated in the organic compound by the electron donor. In this case, the organic compound is preferably a material excellent in transporting the generated electrons. Specifically, the above examples (e.g., the metal complex and the hetero aromatic compound) of the substance forming the electron transporting layer are usable. As the electron donor, any substance exhibiting electron donating property to the organic compound is usable. Specifically, the electron donor is preferably alkali metal, alkaline earth metal and rare earth metal such as lithium, cesium, magnesium, calcium, erbium and ytterbium. The electron donor is also preferably alkali metal oxide and alkaline earth metal oxide such as lithium oxide, calcium oxide, and barium oxide. Moreover, a Lewis base such as magnesium oxide is usable. Further, the organic compound such as tetrathiafulvalene (abbreviation: TTF) is usable.

### Tandem Organic Electroluminescence Device

An exemplary arrangement of the organic EL devices according to the first to third exemplary embodiments may be a so-called tandem organic EL device, in which a plurality of emitting regions are layered via a charge generating layer (occasionally also referred to as an intermediate layer or the like). The tandem organic EL device is exemplified by an organic EL device below.

The organic EL device according to an exemplary arrangement of the first to third exemplary embodiments includes a first emitting unit including the hole transporting zone as a first hole transporting zone and the emitting region as a first emitting region; a first charge generating layer disposed between the first emitting unit and the cathode; and a second emitting unit that is disposed between the first charge generating layer and the cathode and includes a second hole transporting zone and a second emitting region, in which the first hole transporting zone, the first emitting region, the first charge generating layer, the second hole transporting zone, and the second emitting region are disposed in this order, the second hole transporting zone includes a first cathode side organic layer and a second cathode side organic layer, the first cathode side organic layer is in direct contact with the second cathode side organic layer, the first cathode side organic layer and the second cathode side organic layer are disposed between the first charge generating layer and the second emitting region in this order from a side close to the first charge generating layer, a refractive index NL₁ of a constituent material contained in the first cathode side organic layer and a refractive index NL₂ of a constituent material contained in the second cathode side organic layer satisfy a relationship of a numerical formula below (Numerical Formula L), the second emitting region includes one or more emitting layers, the one or more emitting layers include a third emitting layer, the third emitting layer contains a third luminescent compound, and a maximum peak wavelength of the first luminescent compound and a maximum peak wavelength of the third luminescent compound are mutually the same or different.

$${NL}_{1} > {NL}_{2} \qquad ( Numerical Formula L )$$

The tandem organic EL devices according to the first to third exemplary embodiments have an improved luminous efficiency.

In an exemplary arrangement of the tandem organic EL devices of the first to third exemplary embodiments, a maximum peak wavelength of the first luminescent compound and a maximum peak wavelength of the third luminescent compound are different from each other.

In an exemplary arrangement of the tandem organic EL device of the first to third exemplary embodiments, the maximum peak wavelength of the first luminescent compound is shorter than the maximum peak wavelength of the third luminescent compound.

In an exemplary arrangement of the tandem organic EL devices of the first to third exemplary embodiments, one or more maximum peak wavelengths of one or more of the luminescent compounds contained in one or more of the emitting layers in the first emitting region are each shorter than one or more maximum peak wavelengths of one or more of the luminescent compounds contained in one or more of the emitting layers in the second emitting region.

In an exemplary arrangement of the tandem organic EL devices of the first to third exemplary embodiments, the second hole transporting zone further includes a third cathode side organic layer,
the first cathode side organic layer is in direct contact with the second cathode side organic layer, and the second cathode side organic layer is in direct contact with the third cathode side organic layer, and
the first cathode side organic layer, the second cathode side organic layer and the third cathode side organic layer are disposed between the first charge generating layer and the second emitting region in this order from a side close to the first charge generating layer.

In an exemplary arrangement of the organic EL devices of the first to third exemplary embodiments, the tandem organic EL device further includes a third emitting unit and a second charge generating layer. The third emitting unit is disposed between the second emitting unit and the cathode, and the second charge generating layer is disposed between the third emitting unit and the second emitting unit.

In an exemplary arrangement of the tandem organic EL devices of the first to third exemplary embodiments, the third emitting unit includes a third emitting region and a third hole transporting zone.

In an exemplary arrangement of the tandem organic EL devices of the first to third exemplary embodiments, the third emitting region includes one or more emitting layers. The emitting layer(s) included in the third emitting region, the first emitting layer included in the first emitting region and the third emitting layer included in the second emitting region may be the same as or different from each other.

In an exemplary arrangement of the tandem organic EL devices of the first to third exemplary embodiments, the third hole transporting zone includes at least one organic layer. The organic layers included in the first hole transporting zone, the second hole transport zone, and the third hole transport zone may be the same as or different from each other. The organic layers included in the first emitting region and the second emitting region may be the same as or different from each other.

In the tandem organic EL device according to each of the first to third exemplary embodiments, the first charge generating layer and the second charge generating layer each refer to a layer in which holes and electrons are generated when voltage is applied. For instance, when the first charge generating layer includes a plurality of layers, the first charge generating layer preferably includes an N layer disposed close to the anode and through which electrons are injected into the first emitting unit, and a P layer disposed close to the cathode and through which holes are injected into the second emitting unit. For instance, when the second charge generating layer includes a plurality of layers, the second charge generating layer preferably includes an N layer disposed close to the anode and through which electrons are injected into the second emitting unit, and a P layer disposed close to the cathode and through which holes are injected into the third emitting unit. Examples of a material usable for the first charge generating layer and the second charge generating layer include a known material(s) usable for the charge generating layer in the tandem organic EL device.

In an exemplary embodiment, the third emitting layer included in the second emitting region and the emitting layer included in the third emitting region each contain a host material and a luminescent compound. Examples of the host material may be the host materials described in the first exemplary embodiment. Examples of the luminescent compound may be the luminescent compounds described in the first exemplary embodiment.

In an exemplary arrangement of the organic EL devices of the first to third exemplary embodiments, the tandem organic EL device is used for a light-emitting apparatus.

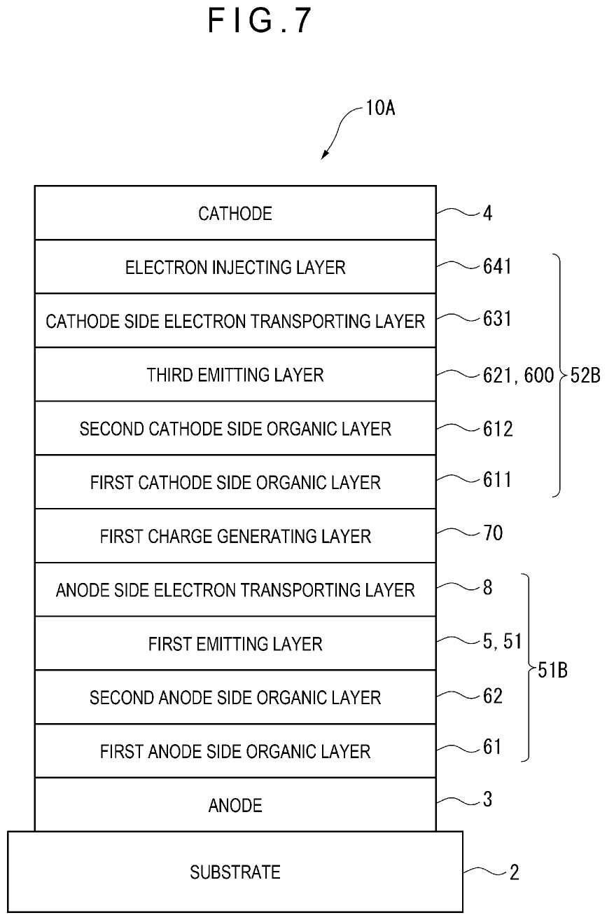

FIG. 7 schematically depicts an organic EL device 10A as an example of the organic EL device according to the first exemplary embodiment. The organic EL device 10A is a tandem organic EL device. The organic EL device 10A includes a substrate 2, a cathode 4, an anode 3, a first charge generating layer 70 interposed between the cathode 4 and the anode 3, a first emitting unit 51B interposed between the first charge generating layer 70 and the anode 3, and a second emitting unit 52B interposed between the first charge generating layer 70 and the cathode 4.

In the organic EL device 10A, the first emitting unit 51B and the second emitting unit 52B are connected in series via the first charge generating layer 70.

The organic EL device 10A includes, as two emitting units, the first emitting unit 51B and the second emitting unit 52B.

The first emitting unit 51B includes a first emitting layer 51 and an anode side electron transporting layer 8, which are layered in this order from a side close to the anode 3. The anode side electron transporting layer 8 corresponds to the electron transporting layer 8 in FIGS. 1 to 6.

The second emitting unit 52B includes a first cathode side organic layer 611, a second cathode side organic layer 612, a third emitting layer 600, a cathode side electron transporting layer 631, and an electron injecting layer 641, which are layered in this order from a side close to the first charge generating layer 70.

The emitting region 5 of the first emitting unit 51B includes the first emitting layer 51, and an emitting region 621 of the second emitting unit 52B includes the third emitting layer 600.

The hole transporting zone included between the anode 3 and the first emitting layer 50 includes a first anode side organic layer 61 and a second anode side organic layer 62.

The hole transporting zone included between the first charge generating layer 70 and the third emitting layer 600 includes the first cathode side organic layer 611 and the second cathode side organic layer 612.

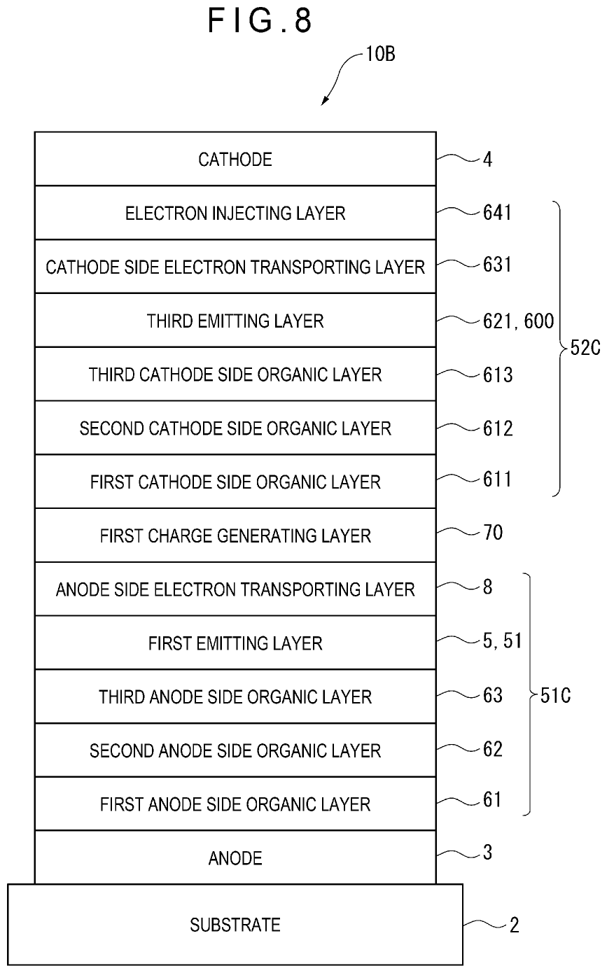

FIG. 8 schematically depicts an organic EL device 10B as another example of the organic EL device according to the first exemplary embodiment.

The organic EL device 10B includes, as two emitting units, a first emitting unit 51C and a second emitting unit 52C.

The organic EL device 10B differs from the organic EL device 10A of FIG. 7 in that the hole transporting zone included between the anode 3 and the first emitting layer 50 includes the first anode side organic layer 61, the second anode side organic layer 62 and a third anode side organic layer 63, and that the hole transporting zone included between the first charge generating layer 70 and the third emitting layer 600 includes the first cathode side organic layer 611, the second cathode side organic layer 612 and a third cathode side organic layer 613.

The arrangements of the tandem organic EL devices depicted in FIGS. 7 and 8 are also applicable to the organic EL device according to the second or third exemplary embodiment.

The organic EL device according to any of the above exemplary embodiments may be a bottom emission type organic EL device.

The organic EL device according to any of the above exemplary embodiments may be a top emission type organic EL device.

### Layer Formation Method

A method for forming each layer of the organic EL device according to any of the above exemplary embodiments is subject to no limitation except for the above particular description. However, known methods of dry film-forming such as vacuum deposition, sputtering, plasma or ion plating and wet film-forming such as spin coating, dipping, flow coating or ink-jet are applicable.

### Film Thickness

The film thickness of each of the organic layers of the organic EL device according to any of the above exemplary embodiments is not limited unless otherwise specified in the above. In general, the thickness preferably ranges from several nanometers to 1 μm because an excessively small film thickness is likely to cause defects (e.g. pin holes) and an excessively large thickness leads to the necessity of applying high voltage and consequent reduction in efficiency.

### Emission Wavelength of Organic EL Device

The organic electroluminescence device according to an exemplary arrangement of any of the exemplary embodiments emits light having a maximum peak wavelength of 500 nm or less when the organic electroluminescence device is driven.

The organic electroluminescence device according to an exemplary arrangement of any of the exemplary embodiments emits light having a maximum peak wavelength in a range from 430 nm to 480 nm when the organic electroluminescence device is driven.

The maximum peak wavelength of the light emitted from the organic EL device when being driven is measured as follows.

Voltage is applied to the organic EL device such that a current density is 10 mA/cm², where spectral radiance spectrum is measured by a spectroradiometer CS-2000 (produced by Konica Minolta, Inc.). A peak wavelength of an emission spectrum, at which the luminous intensity of the obtained spectral radiance spectrum is at the maximum, is measured and defined as a maximum peak wavelength (unit: nm).

### Triplet Energy T₁

A method of measuring triplet energy T₁ is exemplified by a method below.

A measurement target compound is dissolved in EPA (diethylether:isopentane:ethanol=5:5:2 in volume ratio) so as to fall within a range from 10⁻⁵ mol/L to 10⁻⁴ mol/L, and the obtained solution is encapsulated in a quartz cell to provide a measurement sample. A phosphorescence spectrum (ordinate axis: phosphorescent luminous intensity, abscissa axis: wavelength) of the measurement sample was measured at a low temperature (77K). A tangent was drawn to the rise of the phosphorescence spectrum close to the short-wavelength region. An energy amount was calculated by a conversion equation (F1) below on a basis of a wavelength value λ_{edge} [nm] at an intersection of the tangent and the abscissa axis. The calculated energy amount is defined as triplet energy T₁.

$${T}_{1} [ eV ] = 1239 \text{.85} / {λ}_{e d g e} \qquad Conversion Equation ( F1 )$$

The tangent to the rise of the phosphorescence spectrum close to the short-wavelength region is drawn as follows. While moving on a curve of the phosphorescence spectrum from the short-wavelength region to the local maximum value closest to the short-wavelength region among the local maximum values of the phosphorescence spectrum, a tangent is checked at each point on the curve toward the long-wavelength of the phosphorescence spectrum. An inclination of the tangent is increased along the rise of the curve (i.e., a value of the ordinate axis is increased). A tangent drawn at a point of the local maximum inclination (i.e., a tangent at an inflection point) is defined as the tangent to the rise of the phosphorescence spectrum close to the short-wavelength region.

A local maximum point where a peak intensity is 15% or less of the maximum peak intensity of the spectrum is not counted as the above-mentioned local maximum peak intensity closest to the short-wavelength region. The tangent drawn at a point that is closest to the local maximum peak intensity closest to the short-wavelength region and where the inclination of the curve is the local maximum is defined as a tangent to the rise of the phosphorescence spectrum close to the short-wavelength region.

For phosphorescence measurement, a spectrophotofluorometer body F-4500 (manufactured by Hitachi High-Technologies Corporation) is usable. Any apparatus for phosphorescence measurement is usable. A combination of a cooling unit, a low temperature container, an excitation light source and a light-receiving unit may be used for phosphorescence measurement.

### Singlet Energy S₁

A method of measuring a singlet energy S₁ with use of a solution (occasionally referred to as a solution method) is exemplified by a method below.

A toluene solution of a measurement target compound at a concentration ranging from 10⁻⁵ mol/L to 10⁻⁴ mol/L is prepared and put in a quartz cell. An absorption spectrum (ordinate axis: absorption intensity, abscissa axis: wavelength) of the thus-obtained sample is measured at a normal temperature (300K). A tangent is drawn to the fall of the absorption spectrum close to the long-wavelength region, and a wavelength value λ_{edge} (nm) at an intersection of the tangent and the abscissa axis is assigned to a conversion equation (F2) below to calculate singlet energy.

$${S}_{1} [ eV ] = 1239 \text{.85} / {λ}_{e d g e} \qquad Conversion Equation ( F2 )$$

Any apparatus for measuring absorption spectrum is usable. For instance, a spectrophotometer (U3310 manufactured by Hitachi, Ltd.) is usable.

The tangent to the fall of the absorption spectrum close to the long-wavelength region is drawn as follows. While moving on a curve of the absorption spectrum from the local maximum value closest to the long-wavelength region, among the local maximum values of the absorption spectrum, in a long-wavelength direction, a tangent at each point on the curve is checked. An inclination of the tangent is decreased and increased in a repeated manner as the curve falls (i.e., a value of the ordinate axis is decreased). A tangent drawn at a point where the inclination of the curve is the local minimum closest to the long-wavelength region (except when absorbance is 0.1 or less) is defined as the tangent to the fall of the absorption spectrum close to the long-wavelength region.

The local maximum absorbance of 0.2 or less is not counted as the above-mentioned local maximum absorbance closest to the long-wavelength region.

### Method of Measuring Electron Mobility

The electron mobility can be measured according to an impedance measurement using a mobility evaluation device produced by the following steps. The device for mobility evaluation is produced, for instance, according to the following steps.

A compound Target, which is to be measured for an electron mobility, is vapor-deposited on a glass substrate having an aluminum electrode (anode) so as to cover the aluminum electrode, thereby forming a measurement target layer. A compound ET-A below is vapor-deposited on this measurement target layer to form an electron transporting layer. LiF is vapor-deposited on this formed electron transporting layer to form an electron injecting layer. Metal aluminum (Al) is vapor-deposited on this formed electron injecting layer to form a metal cathode.

An arrangement of the mobility evaluation device above is roughly shown as follows.
glass/Al(50)/Target(200)/ET-A(10)/LiF(1)/Al(50)

Numerals in parentheses represent a film thickness (nm).

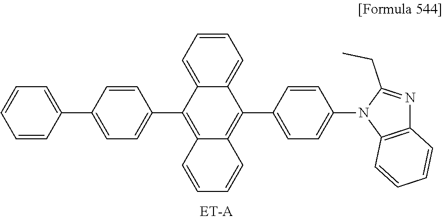 [SMILES: CCc1nc2ccccc2n1-c1ccc(-c2c3ccccc3c(-c3ccc(-c4ccccc4)cc3)c3ccccc23)cc1]

The mobility evaluation device for the electron mobility is set in an impedance measurement apparatus to perform an impedance measurement. In the impedance measurement, a measurement frequency is swept from 1 Hz to 1 MHz. At this time, an alternating current amplitude of 0.1 V and a direct current voltage V are simultaneously applied to the device. A modulus M is calculated from a measured impedance Z using a relationship of a calculation formula (C1) below.

$$M = j ω Z \qquad Calculation formula ( C1 )$$

In the calculation formula (C1), j is an imaginary unit whose square is −1 and ω is an angular frequency [rad/s].

In a bode plot in which an imaginary part of the modulus M is represented by an ordinate axis and the frequency [Hz] is represented by an abscissa axis, an electrical time constant τ of the mobility evaluation device is obtained from a frequency fmax showing a peak using a calculation formula (C2) below.

$$⊤ = 1 / ( 2 π f max ) \qquad Calculation formula ( C2 )$$

π in the calculation formula (C2) is a symbol representing a circumference ratio.

An electron mobility μe is calculated from a relationship of a calculation formula (C3-1) below using τ.

$$μ e = {d}^{2} / ( {V}_{⊤} ) \qquad Calculation formula ( C3 - 1 )$$

d in the calculation formula (C3-1) is a total film thickness of organic thin film(s) forming the device. In a case of the arrangement of the mobility evaluation device for the electron mobility, d=210 [nm] is satisfied.

### Method of Measuring Hole Mobility

The hole mobility can be measured according to an impedance measurement using a mobility evaluation device produced by the following steps. The device for mobility evaluation is produced, for instance, according to the following steps.

A compound HA-2 below is vapor-deposited on a glass substrate having an ITO transparent electrode (anode) so as to cover the transparent electrode, thereby forming a hole injecting layer. A compound HT-A below is vapor-deposited on this formed hole injecting layer to form a hole transporting layer. Subsequently, a compound Target, which is to be measured for a hole mobility, is vapor-deposited to form a measurement target layer. Metal aluminum (Al) is vapor-deposited on this measurement target layer to form a metal cathode.

An arrangement of the mobility evaluation device above is roughly shown as follows.
ITO(130)/HA-2(5)/HT-A(10)/Target(200)/Al(80)

Numerals in parentheses represent a film thickness (nm).

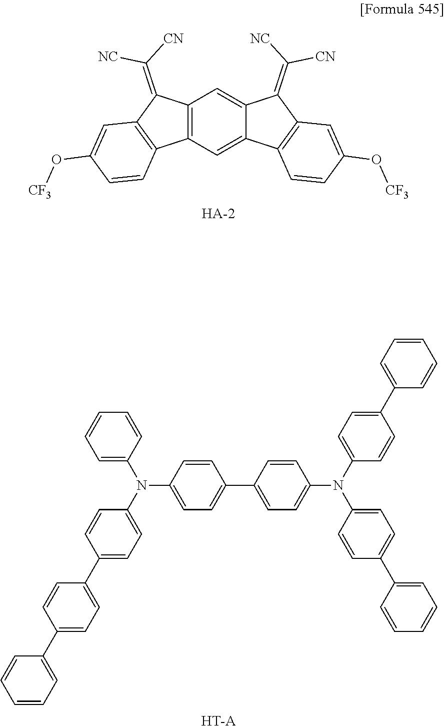 [SMILES: N#CC(C#N)=C1c2cc(OC(F)(F)F)ccc2-c2cc3c(cc21)C(=C(C#N)C#N)c1cc(OC(F)(F)F)ccc1-3 ; "HA-2" ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; "HT-A"]

The mobility evaluation device for the hole mobility is set in an impedance measurement apparatus to perform an impedance measurement. In the impedance measurement, a measurement frequency is swept from 1 Hz to 1 MHz. At this time, an alternating current amplitude of 0.1 V and a direct current voltage V are applied to the device. A modulus M is calculated from a measured impedance Z using the relationship of the calculation formula (C1).

In a bode plot in which an imaginary part of the modulus M is represented by an ordinate axis and the frequency [Hz] is represented by an abscissa axis, an electrical time constant τ of the mobility evaluation device is obtained from a frequency fmax showing a peak using the calculation formula (C2).

A hole mobility μh is calculated from a relationship of a calculation formula (C3-2) below using T obtained from the calculation formula (C2).

$$μ h = {d}^{2} / ( {V}_{⊤} ) \qquad Calculation formula ( C3 - 2 )$$

d in the calculation formula (C3-2) is a total film thickness of organic thin film(s) forming the device. In a case of the arrangement of the mobility evaluation device for the hole mobility, d=215 [nm] is satisfied.

The electron mobility and the hole mobility herein are each a value obtained in a case where a square root of an electric field intensity meets E^{1/2}=500 [V^{1/2}/cm^{1/2}]. The square root of the electric field intensity, E^{1/2}, can be calculated from a relationship of a calculation formula (C4) below.

$${E}^{1 / 2} = {V}^{1 / 2} / {d}^{1 / 2} \qquad Calculation formula ( C4 )$$

For the impedance measurement, a 1260 type by Solartron Analytical is used as the impedance measurement apparatus, and for a higher accuracy, a 1296 type dielectric constant measurement interface by Solartron Analytical can be used together therewith.

### Fourth Exemplary Embodiment

### Electronic Device

An electronic device according to a fourth exemplary embodiment is installed with the organic EL device according to any of the above exemplary embodiments. Examples of the electronic device include a display device and a light-emitting apparatus. Examples of the display device include a display component (e.g., an organic EL panel module), TV, mobile μhone, tablet and personal computer. Examples of the light-emitting apparatus include an illuminator and a vehicle light.

In an exemplary arrangement of the electronic device according to the fourth exemplary embodiment, the light-emitting apparatus is installed with the tandem organic EL device according to the above exemplary embodiment. In an exemplary arrangement of the electronic device according to the fourth exemplary embodiment, the light-emitting apparatus preferably includes the tandem organic EL device according to the above exemplary embodiment and a color conversion layer. The light-emitting apparatus preferably includes a color filter. The color conversion layer is preferably disposed between the tandem organic EL device and the color filter. The color conversion layer preferably contains a substance that emits light by absorbing light. The substance that emits light by absorbing light is preferably a quantum dot. In the light-emitting apparatus, the color conversion layer is preferably disposed to be irradiated with light emission from the tandem organic EL device.

In an exemplary arrangement of the electronic device according to the fourth exemplary embodiment, the display device is installed with the light-emitting apparatus. The light-emitting apparatus also can be used for a display device, for instance, as a backlight of the display device.

### Modification of Embodiments

The scope of the invention is not limited by the above-described exemplary embodiments but includes any modification and improvement as long as such modification and improvement are compatible with the invention.

For instance, the number of emitting layers is not limited to one or two, and more than two emitting layers may be layered. For instance, the rest of the emitting layers may be a fluorescent emitting layer or a phosphorescent emitting layer with use of emission caused by electron transfer from the triplet excited state directly to the ground state.

Further, for instance, a blocking layer is optionally provided adjacent to a side of the emitting layer close to the cathode. The blocking layer provided in direct contact with the side of the emitting layer close to the cathode preferably blocks at least one of holes or excitons.

For instance, when the blocking layer is provided in contact with the side of the emitting layer close to the cathode, the blocking layer permits transport of electrons, and blocks holes from reaching a layer provided closer to the cathode (e.g., the electron transporting layer) beyond the blocking layer. When the organic EL device includes the electron transporting layer, the blocking layer may be disposed between the emitting layer and the electron transporting layer.

Alternatively, the blocking layer may be provided adjacent to the emitting layer so that the excitation energy does not leak out from the emitting layer toward neighboring layer(s). The blocking layer blocks excitons generated in the emitting layer from being transferred to a layer(s) (e.g., the electron transporting layer) closer to the electrode(s) than the blocking layer. The emitting layer is preferably in direct contact with the blocking layer.

The specific structure, shape, and the like of the components in the invention may be designed in any manner as long as an object of the invention can be achieved.

The invention will be described in further detail with reference to Examples. The scope of the invention is by no means limited to Examples.

### EXAMPLES

### Compounds

Structures of compounds as the first organic material used for producing organic EL devices in Examples are shown below.

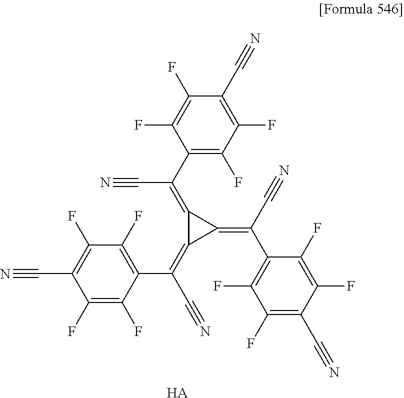 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; "HA"]

Structures of compounds, as the second organic material, the second hole transporting zone material and the third hole transporting zone material, used for producing organic EL devices in Examples are shown below.

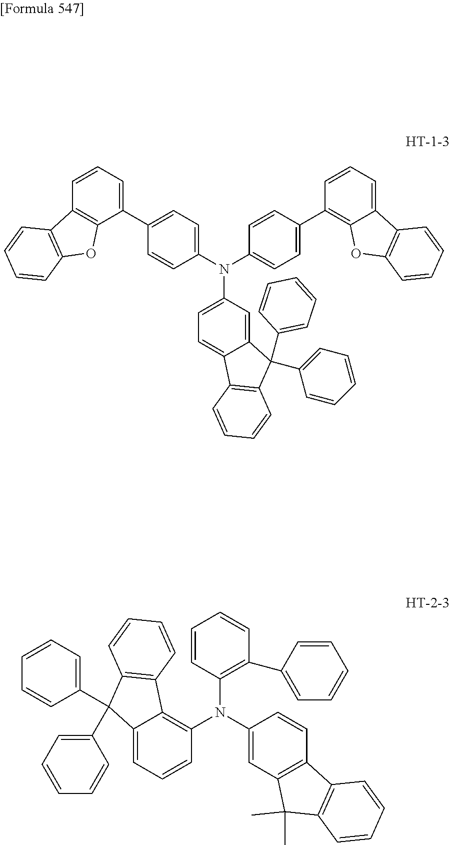 [SMILES: c1ccc(C2(c3ccccc3)c3ccccc3-c3ccc(N(c4ccc(-c5cccc6c5oc5ccccc56)cc4)c4ccc(-c5cccc6c5oc5ccccc56)cc4)cc32)cc1 ; CC1(C)c2ccccc2-c2ccc(N(c3ccccc3-c3ccccc3)c3cccc4c3-c3ccccc3C4(c3ccccc3)c3ccccc3)cc21]

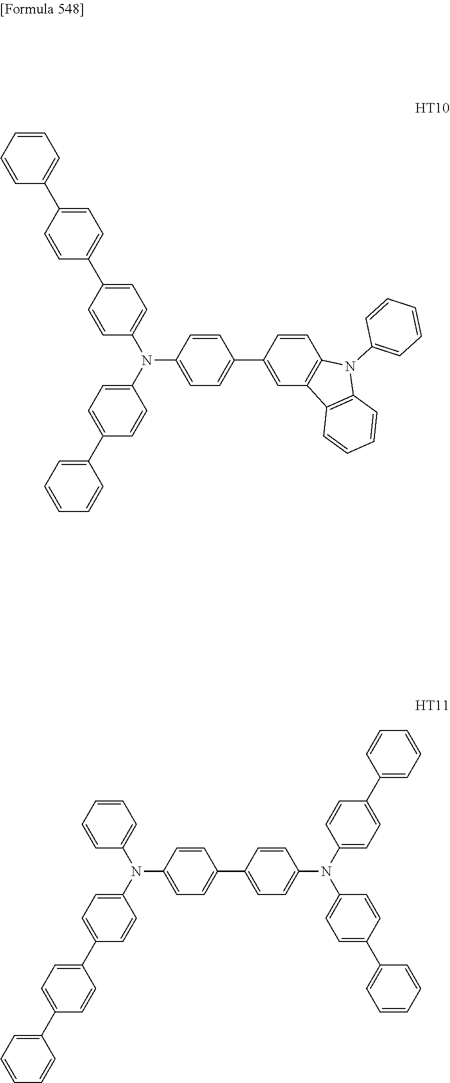 [SMILES: c1ccc(-c2ccc(-c3ccc(N(c4ccc(-c5ccccc5)cc4)c4ccc(-c5ccc6c(c5)c5ccccc5n6-c5ccccc5)cc4)cc3)cc2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

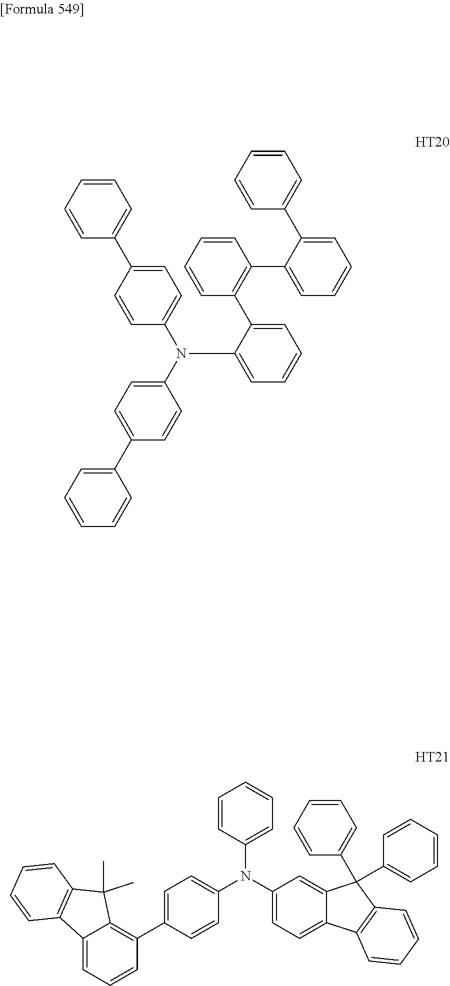 [SMILES: c1ccc(-c2ccc(N(c3ccc(-c4ccccc4)cc3)c3ccccc3-c3ccccc3-c3ccccc3-c3ccccc3)cc2)cc1 ; CC1(C)c2ccccc2-c2cccc(-c3ccc(N(c4ccccc4)c4ccc5c(c4)C(c4ccccc4)(c4ccccc4)c4ccccc4-5)cc3)c21]

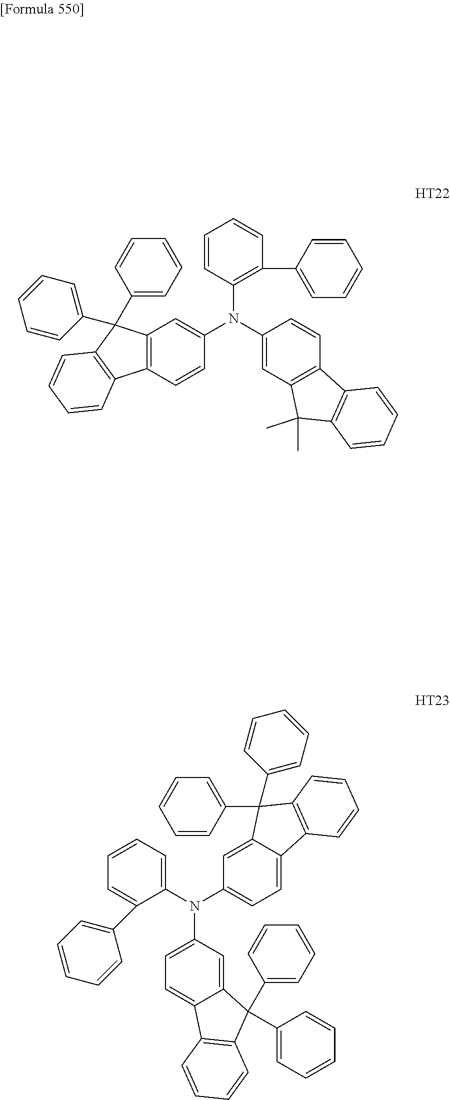 [SMILES: CC1(C)c2ccccc2-c2ccc(N(c3ccc4c(c3)C(c3ccccc3)(c3ccccc3)c3ccccc3-4)c3ccccc3-c3ccccc3)cc21 ; c1ccc(-c2ccccc2N(c2ccc3c(c2)C(c2ccccc2)(c2ccccc2)c2ccccc2-3)c2ccc3c(c2)C(c2ccccc2)(c2ccccc2)c2ccccc2-3)cc1]

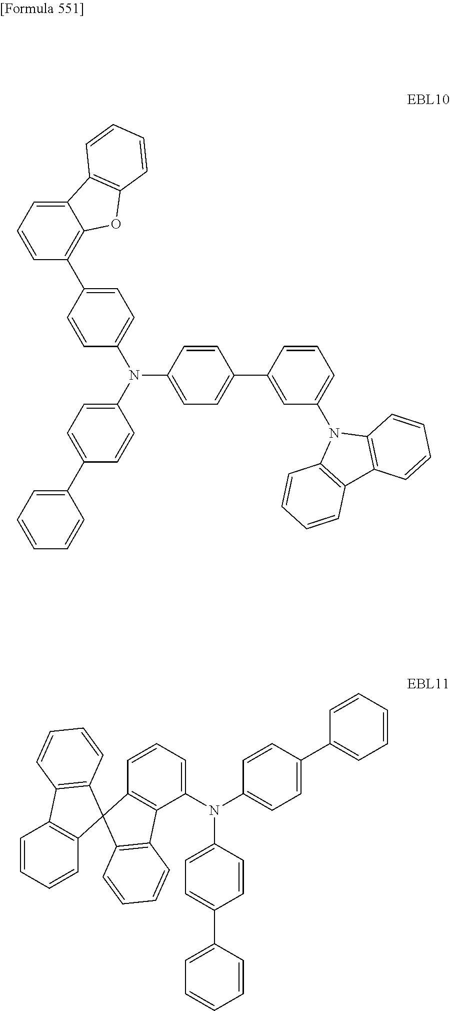 [SMILES: c1ccc(-c2ccc(N(c3ccc(-c4cccc(-n5c6ccccc6c6ccccc65)c4)cc3)c3ccc(-c4cccc5c4oc4ccccc45)cc3)cc2)cc1 ; c1ccc(-c2ccc(N(c3ccc(-c4ccccc4)cc3)c3cccc4c3-c3ccccc3C43c4ccccc4-c4ccccc43)cc2)cc1]

Structures of compounds, as the first host material, the first additional host material, and the first luminescent compound, used for producing organic EL devices in Examples are shown below.

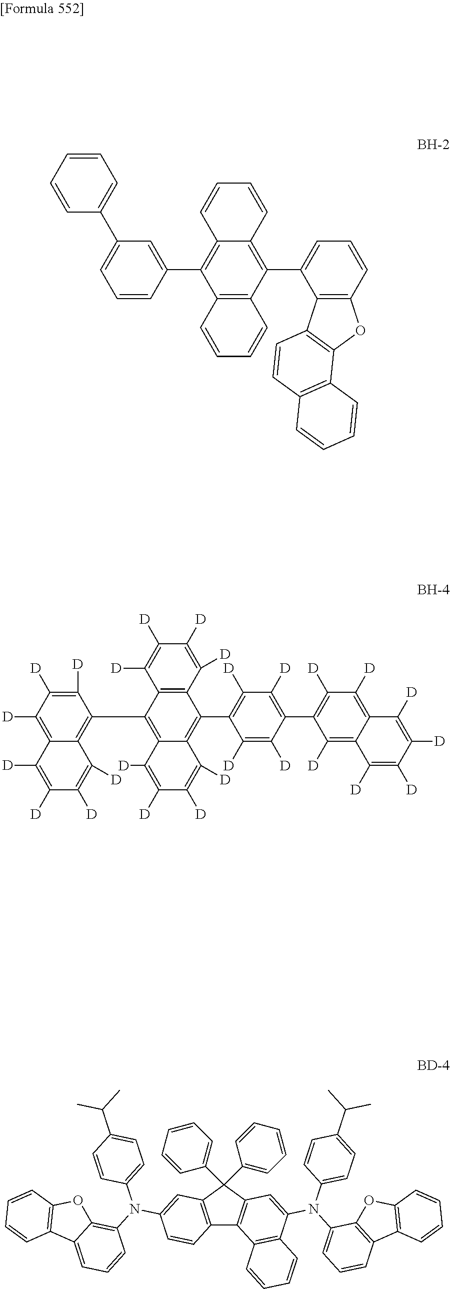 [SMILES: c1ccc(-c2cccc(-c3c4ccccc4c(-c4cccc5oc6c7ccccc7ccc6c45)c4ccccc34)c2)cc1 ; [2H]c1c([2H])c(-c2c3c([2H])c([2H])c([2H])c([2H])c3c(-c3c([2H])c([2H])c([2H])c4c([2H])c([2H])c([2H])c([2H])c34)c3c([2H])c([2H])c([2H])c([2H])c23)c([2H])c([2H])c1-c1c([2H])c([2H])c2c([2H])c([2H])c([2H])c([2H])c2c1[2H] ; CC(C)c1ccc(N(c2ccc3c(c2)C(c2ccccc2)(c2ccccc2)c2cc(N(c4ccc(C(C)C)cc4)c4cccc5c4oc4ccccc45)c4ccccc4c2-3)c2cccc3c2oc2ccccc23)cc1]

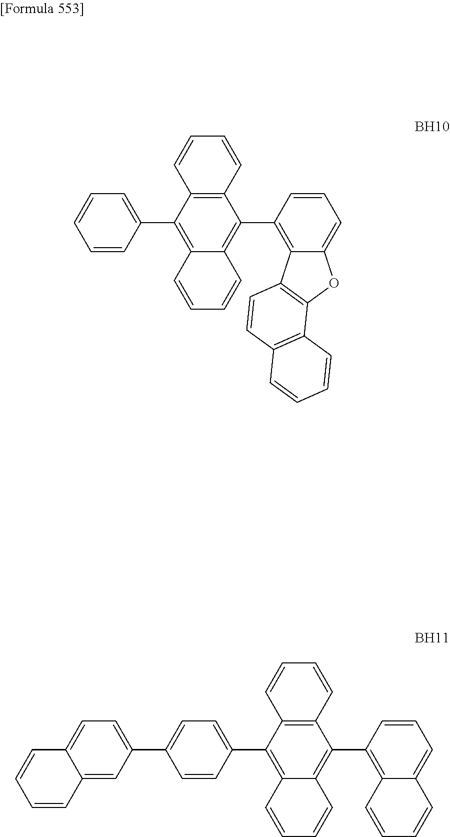

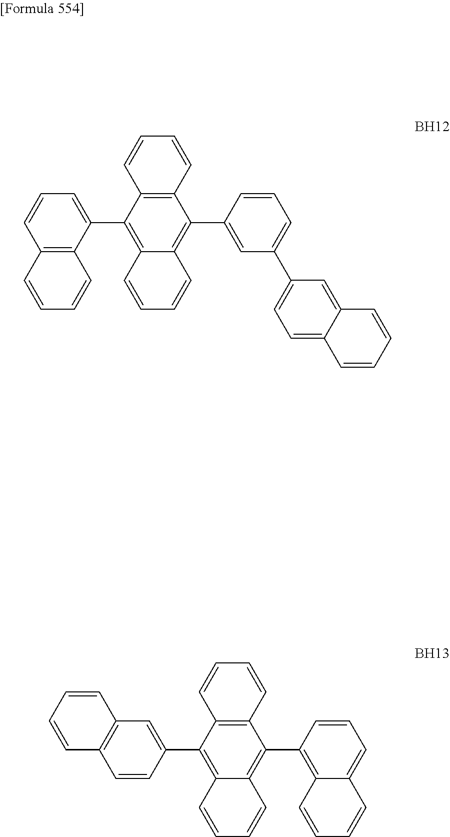

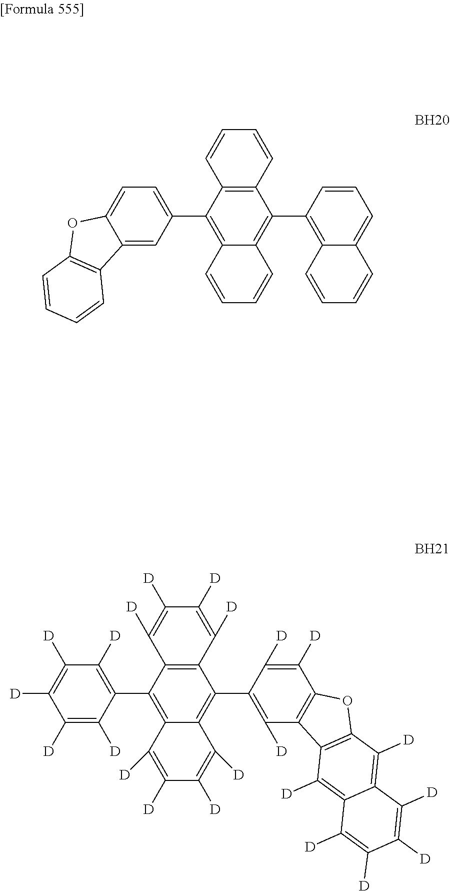 [SMILES: c1ccc2c(-c3c4ccccc4c(-c4ccc5oc6ccccc6c5c4)c4ccccc34)cccc2c1 ; [2H]c1c([2H])c([2H])c(-c2c3c([2H])c([2H])c([2H])c([2H])c3c(-c3c([2H])c([2H])c4oc5c([2H])c6c([2H])c([2H])c([2H])c([2H])c6c([2H])c5c4c3[2H])c3c([2H])c([2H])c([2H])c([2H])c23)c([2H])c1[2H]]

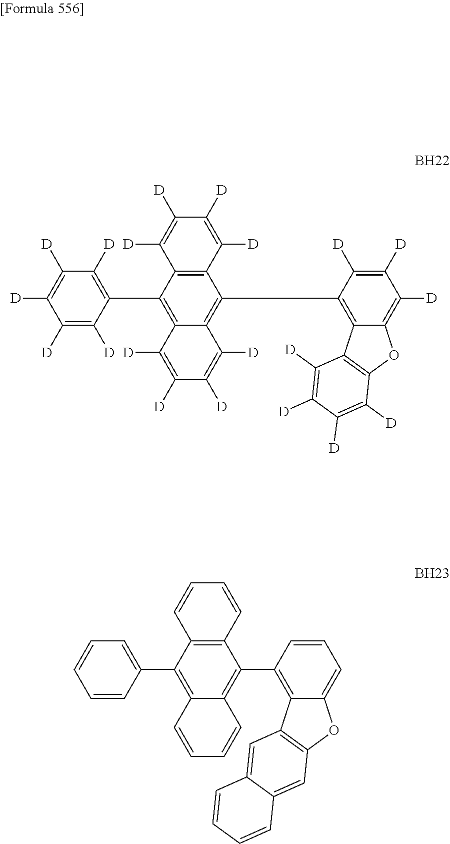 [SMILES: [2H]c1c([2H])c([2H])c(-c2c3c([2H])c([2H])c([2H])c([2H])c3c(-c3c([2H])c([2H])c([2H])c4oc5c([2H])c([2H])c([2H])c([2H])c5c34)c3c([2H])c([2H])c([2H])c([2H])c23)c([2H])c1[2H] ; c1ccc(-c2c3ccccc3c(-c3cccc4oc5cc6ccccc6cc5c34)c3ccccc23)cc1]

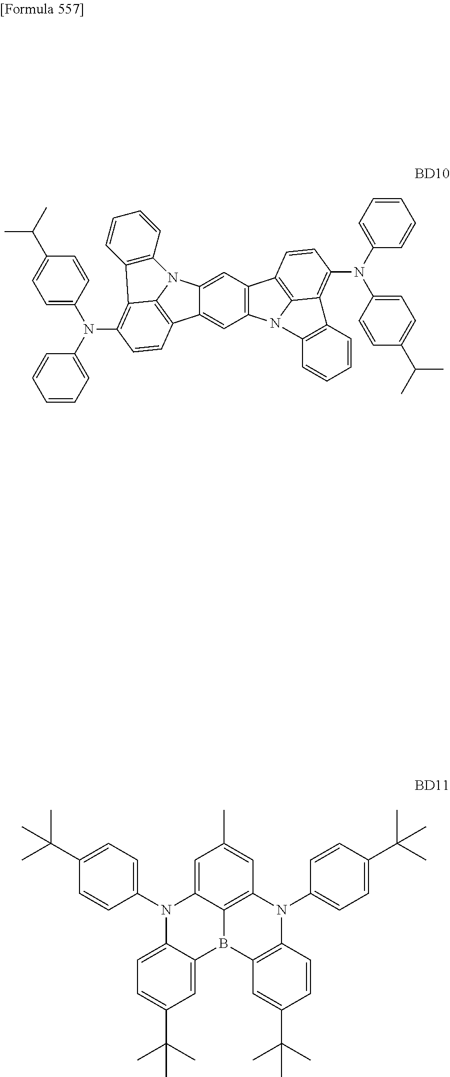

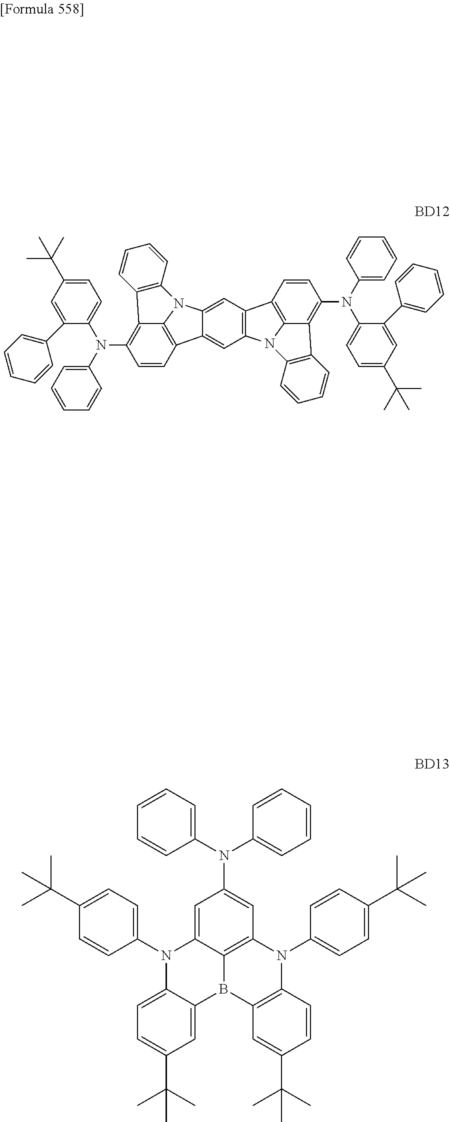 [SMILES: CC(C)(C)c1ccc(N(c2ccccc2)c2ccc3c4cc5c(cc4n4c6ccccc6c2c34)c2ccc(N(c3ccccc3)c3ccc(C(C)(C)C)cc3-c3ccccc3)c3c4ccccc4n5c23)c(-c2ccccc2)c1 ; CC(C)(C)c1ccc(N2c3ccc(C(C)(C)C)cc3B3c4cc(C(C)(C)C)ccc4N(c4ccc(C(C)(C)C)cc4)c4cc(N(c5ccccc5)c5ccccc5)cc2c43)cc1]

Structures of the other compounds used for producing organic EL devices in Examples are shown below.

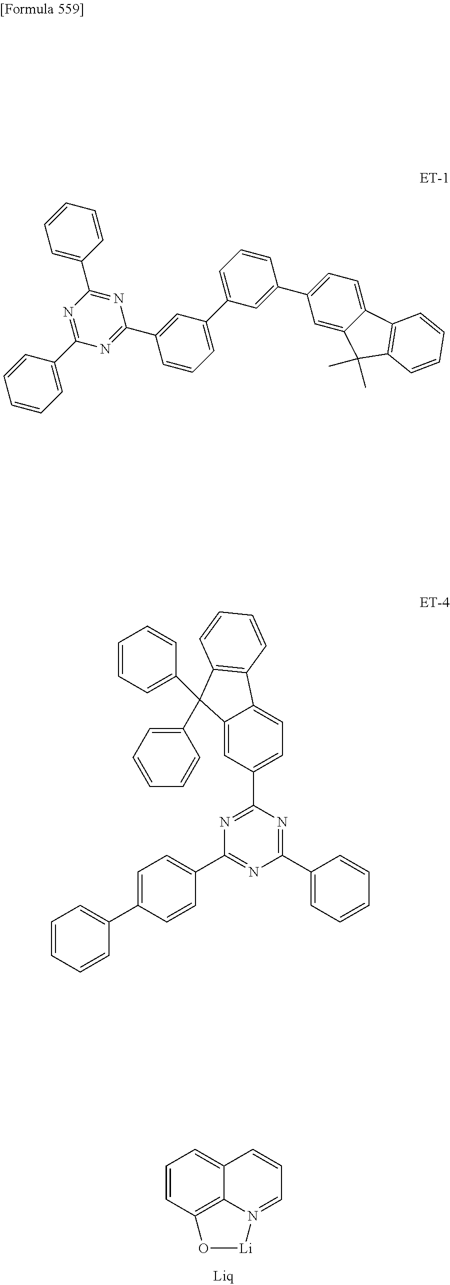

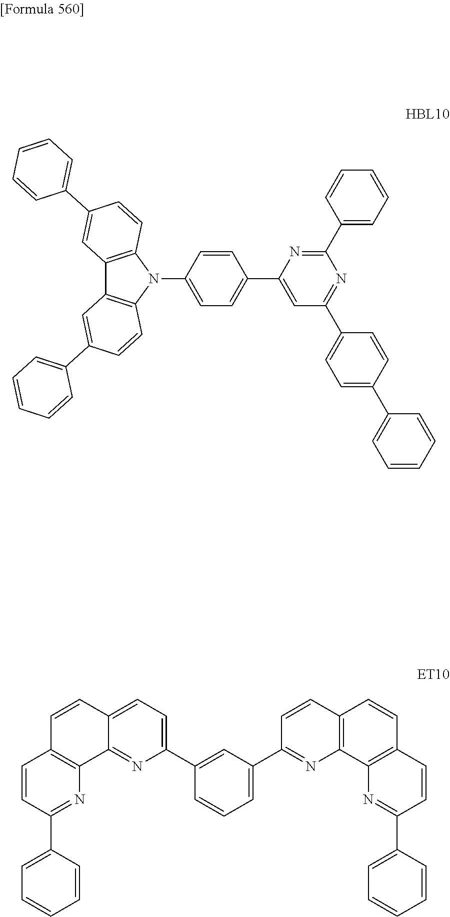 [SMILES: c1ccc(-c2ccc(-c3cc(-c4ccc(-n5c6ccc(-c7ccccc7)cc6c6cc(-c7ccccc7)ccc65)cc4)nc(-c4ccccc4)n3)cc2)cc1 ; c1ccc(-c2ccc3ccc4ccc(-c5cccc(-c6ccc7ccc8ccc(-c9ccccc9)nc8c7n6)c5)nc4c3n2)cc1]

### Production of Organic EL Device

The organic EL devices were produced and evaluated as follows.

### Example 1

A glass substrate (size: 25 mm×75 mm×1.1 mm thick, produced by Geomatec Co., Ltd.) having an indium tin oxide (ITO) transparent electrode (anode) was ultrasonic-cleaned in isopropyl alcohol for five minutes, and then UV-ozone-cleaned for 30 minutes. The film thickness of the ITO transparent electrode was 130 nm.

After the glass substrate having the transparent electrode line was cleaned, the glass substrate was mounted on a substrate holder of a vacuum deposition apparatus. First, a compound HT-1-3 and a compound HA were co-deposited on a surface of the glass substrate, where the transparent electrode line was provided, to cover the transparent electrode, thereby forming a 10-nm-thick first anode side organic layer (occasionally also referred to as a hole injecting layer). The ratios of the compound HT-1-3 and the compound HA in the first anode side organic layer were 97 mass % and 3 mass %, respectively.

A compound HT-2-3 was vapor-deposited on the first anode side organic layer to form a 40-nm-thick second anode side organic layer (occasionally also referred to as a first hole transporting layer).

A compound BH-2 (first host material), a compound BH-4 (first additional host material) and a compound BD-4 (first luminescent compound) were co-deposited on the second anode side organic layer, thereby forming a 25-nm-thick first emitting layer. The ratios of the compound BH-2, the compound BH-4 and the compound BD-4 in the first emitting layer were 50 mass %, 47 mass %, 3 mass %, respectively.

A compound ET-1 was vapor-deposited on the first emitting layer to form a 5-nm-thick first electron transporting layer (occasionally also referred to as a hole blocking layer (HBL)).

A compound ET-4 and a compound Liq were co-deposited on the first electron transporting layer such that the ratio of the compound ET4 was 50 mass % and the ratio of the compound Liq was 50 mass % to form a 20-nm-thick second electron transporting layer (ET).

A compound LiF was vapor-deposited on the second electron transporting layer to form a 1-nm-thick electron injecting layer.

Metal (Al) was vapor-deposited on the electron injecting layer to form an 80-nm-thick cathode.

A device arrangement of the organic EL device in Example 1 is roughly shown as follows.
ITO(130)/HT-1-3:HA(10,97%:3%)/HT-2-3(40)/BH-2:BH-4:BD-4(25,50%:47%:3%)/ET-1(5)/ET-4:Liq(20,50%:50%)/LiF(1)/Al(80)

Numerals in parentheses represent a film thickness (unit: nm).

The numerals (97%:3%) represented by percentage in the same parentheses indicate a ratio (mass %) between the compound HT-1-3 and the compound HA in the first anode side organic layer, the numerals (50%:47%:3%) represented by percentage in the same parentheses indicate a ratio (mass %) between the first host material (compound BH-2), the first additional host material (compound BH-4), and the first luminescent compound (compound BD-4) in the first emitting layer, and the numerals (50%:50%) represented by percentage in the same parentheses indicate a ratio (mass %) between the compound ET-4 and the compound Liq in the second electron transporting layer.

### Reference Example 1

An organic EL device of Reference Example 1 was produced as in the organic EL device of Example 1 except that the compound BH-4 and the compound BD-4 were co-deposited on the second anode side organic layer to form the first emitting layer. The ratios of the compound BH-4 and the compound BD-4 in the first emitting layer were 97 mass % and 3 mass %, respectively.

### Reference Example 2

An organic EL device of Reference Example 2 was produced as in the organic EL device of Example 1 except that the compound BH-2 and the compound BD-4 were co-deposited on the second anode side organic layer to form the first emitting layer. The ratios of the compound BH-2 and the compound BD-4 in the first emitting layer were 97 mass % and 3 mass %, respectively.

### Comparative Example 1

An organic EL device of Comparative Example 1 was produced as in the organic EL device of Example 1 except that the compound HT-1-3 was vapor-deposited on the first anode side organic layer to form a 35-nm-thick second anode side organic layer, that the compound HT-2-3 was vapor-deposited on this second anode side organic layer to form a 5-nm-thick third anode side organic layer, and that the first emitting layer was formed on this third anode side organic layer.

### Comparative Example 2

An organic EL device of Comparative Example 2 was produced as in the organic EL device of Reference Example 1 except that the compound HT-1-3 was vapor-deposited on the first anode side organic layer to form a 35-nm-thick second anode side organic layer, that the compound HT-2-3 was vapor-deposited on this second anode side organic layer to form a 5-nm-thick third anode side organic layer, and that the first emitting layer was formed on this third anode side organic layer.

### Comparative Example 3

An organic EL device of Comparative Example 3 was produced as in the organic EL device of Reference Example 2 except that the compound HT-1-3 was vapor-deposited on the first anode side organic layer to form a 35-nm-thick second anode side organic layer, that the compound HT-2-3 was vapor-deposited on this second anode side organic layer to form a 5-nm-thick third anode side organic layer, and that the first emitting layer was formed on this third anode side organic layer.

### Example 2

A glass substrate (size: 25 mm×75 mm×1.1 mm thick, produced by Geomatec Co., Ltd.) having an indium tin oxide (ITO) transparent electrode (anode) was ultrasonic-cleaned in isopropyl alcohol for five minutes, and then UV-ozone-cleaned for 30 minutes. The film thickness of the ITO transparent electrode was 130 nm.

After the glass substrate having the transparent electrode line was cleaned, the glass substrate was mounted on a substrate holder of a vacuum deposition apparatus. First, a compound HT10 and a compound HA were co-deposited on a surface of the glass substrate, where the transparent electrode line was provided, to cover the transparent electrode, thereby forming a 10-nm-thick first anode side organic layer (occasionally also referred to as a hole injecting layer). The ratios of the compound HT10 and the compound HA in the first anode side organic layer were 97 mass % and 3 mass %, respectively.

A compound HT20 was vapor-deposited on the first anode side organic layer to form a 40-nm-thick second anode side organic layer (occasionally also referred to as a first hole transporting layer).

A compound EBL10 was vapor-deposited on the second anode side organic layer to form a 10-nm-thick third anode side organic layer (occasionally also referred to as a second hole transporting layer).

A compound BH10 (first host material), a compound BH20 (first additional host material) and a compound BD10 (first luminescent compound) were co-deposited on the second anode side organic layer, thereby forming a 25-nm-thick first emitting layer. The ratios of the compound BH10, the compound BH20 and the compound BD10 in the first emitting layer were 49.5 mass %, 49.5 mass %, 1 mass %, respectively.

A compound HBL10 was vapor-deposited on the first emitting layer to form a 10-nm-thick first electron transporting layer (occasionally also referred to as a hole blocking layer (HBL)).

A compound ET10 and Li were co-deposited on the first electron transporting layer such that the ratio of the compound ET10 was 96 mass % and the ratio of Li was 4 mass % to form a 15-nm-thick second electron transporting layer (ET).

A compound LiF was vapor-deposited on the second electron transporting layer to form a 1-nm-thick electron injecting layer.

Metal (Al) was vapor-deposited on the electron injecting layer to form an 80-nm-thick cathode.

A device arrangement of the organic EL device in Example 2 is roughly shown as follows.
ITO(130)/HT10:HA(10,97%:3%)/HT20(40)/EBL10(10)/BH10:BH20:BD10(25,49.5%: 49.5%:1%)/HBL10(10)/ET10:Li(15,96%:4%)/LiF(1)/Al(80)

Numerals in parentheses represent a film thickness (unit: nm).

The numerals (97%:3%) represented by percentage in the same parentheses indicate a ratio (mass %) between the compound HT10 and the compound HA in the first anode side organic layer, the numerals (49.5%:49.5%:1%) represented by percentage in the same parentheses indicate a ratio (mass %) between the first host material (compound BH10), the first additional host material (compound BH20), and the first luminescent compound (compound BD10) in the first emitting layer, and the numerals (96%:4%) represented by percentage in the same parentheses indicate a ratio (mass %) between the compound ET10 and Li in the second electron transporting layer.

### Examples 3 to 5

Organic EL devices of Examples 3 to 5 were each produced as in the organic EL device of Example 2 except that compounds listed in Table 2 were respectively used as compounds contained in the first anode side organic layer, the second anode side organic layer, the third anode side organic layer, and the first emitting layer.

### Comparative Example 4

An organic EL device of Comparative Example 4 was produced as in the organic EL device of Example 2 except that compounds listed in Table 2 were respectively used as compounds contained in the first anode side organic layer, the second anode side organic layer, the third anode side organic layer, and the first emitting layer.

### Evaluation on Organic EL Devices

The produced organic EL devices were evaluated as follows. Tables 1 and 2 show evaluation results.

### Drive Voltage

A voltage (unit: V) was measured when current was applied to between the anode and the cathode such that a current density was 10 mA/cm².

### External Quantum Efficiency EQE

Voltage was applied to the organic EL devices such that a current density was 10 mA/cm², where spectral radiance spectrum was measured by a spectroradiometer CS-2000 (produced by Konica Minolta, Inc.). The external quantum efficiency EQE (unit: %) was calculated based on the obtained spectral radiance spectra, assuming that the spectra were provided under a Lambertian radiation.

### Lifetime LT95

Voltage was applied to the produced organic EL devices so that a current density was 50 mA/cm², where a time (LT95 (unit: hr)) elapsed before a luminance intensity was reduced to 95% of the initial luminance intensity was measured. The luminance intensity was measured by a spectroradiometer CS-2000 (produced by Konica Minolta, Inc.).

TABLE 1

| | Hole transporting zone | | | | | | | | | | |
|---|---|---|---|---|---|---|---|---|---|---|---|
| | | | | | | | | Third anode side | | | |
| | | | | | | | | organic layer | | | |
| | | | | | Second anode side organic layer | | | Third | | Difference in | Emitting region |
| | First anode side organic layer | | | | Second hole | | | hole | | refractive | First emitting |
| | First | Second | Refractive | Film | transporting | Refractive | Film | transporting | Film | index | layer |
| | organic | organic | index | thickness | zone | index | thickness | zone | thickness | NM₁- | First host |
| | material | material | NM₁ | [nm] | material | NM₂ | [nm] | material | [nm] | NM₂ | material |
| Ex. 1 | HA | HT-1-3 | 1.94 | 10 | HT-2-3 | 1.78 | 40 | — | — | 0.16 | BH-2 |
| Reference | HA | HT-1-3 | 1.94 | 10 | HT-2-3 | 1.78 | 40 | — | — | 0.16 | BH-4 |
| Ex. 1 | | | | | | | | | | | |
| Reference | HA | HT-1-3 | 1.94 | 10 | HT-2-3 | 1.78 | 40 | — | — | 0.16 | BH-2 |
| Ex. 2 | | | | | | | | | | | |
| Comp. 1 | HA | HT-1-3 | 1.94 | 10 | HT-1-3 | 1.94 | 35 | HT-2-3 | 5 | 0.00 | BH-2 |
| Comp. 2 | HA | HT-1-3 | 1.94 | 10 | HT-1-3 | 1.94 | 35 | HT-2-3 | 5 | 0.00 | BH-4 |
| Comp. 3 | HA | HT-1-3 | 1.94 | 10 | HT-1-3 | 1.94 | 35 | HT-2-3 | 5 | 0.00 | BH-2 |

| | Emitting region | | | | | | | | | |
|---|---|---|---|---|---|---|---|---|---|---|
| | First emitting | | | Electron transporting zone | | | | | | |
| | layer | | | First electron | | Second electron | | | | |
| | | First | Film | transporting layer | | transporting layer | | Evaluation on Device | | |
| | First additional | luminescent | thickness | Compound | Film thickness | Compound | Film thickness | Voltage | EQE | LT95 |
| | host material | compound | [nm] | Name | [nm] | Name | [nm] | [V] | [%] | [hr] |
| Ex. 1 | BH-4 | BD-4 | 25 | ET-1 | 5 | ET-4, Liq | 20 | 3.4 | 9.6 | 210 |
| Reference | — | BD-4 | 25 | ET-1 | 5 | ET-4, Liq | 20 | 4.2 | 9.5 | 180 |
| Ex. 1 | | | | | | | | | | |
| Reference | — | BD-4 | 25 | ET-1 | 5 | ET-4, Liq | 20 | 3.5 | 9.4 | 40 |
| Ex. 2 | | | | | | | | | | |
| Comp. 1 | BH-4 | BD-4 | 25 | ET-1 | 5 | ET-4, Liq | 20 | 3.3 | 8.9 | 140 |
| Comp. 2 | — | BD-4 | 25 | ET-1 | 5 | ET-4, Liq | 20 | 3.8 | 9.0 | 210 |
| Comp. 3 | — | BD-4 | 25 | ET-1 | 5 | ET-4, Liq | 20 | 3.2 | 8.9 | 70 |

Evaluation results of Example 1, in which the relationship of the numerical formula NM (NM₁>NM₂) was satisfied and the first emitting layer contained two types of the host material (first host material and first additional host material), are as follows.

Example 1 emitted light with higher efficiency and had a longer lifetime than Comparative Example 1 in which the relationship of the numerical formula NM was not satisfied.

Example 1 emitted light with higher efficiency and had a longer lifetime than Reference Examples 1 and 2 each in which the emitting layer contained a single type of the host material. Further, Example 1 was driven at a lower voltage than Reference Examples 1 and 2.

Example 1 emitted light with higher efficiency than Comparative Examples 2 and 3 each in which the relationship of the numerical formula NM was not satisfied and the emitting layer contained a single type of the host material.

TABLE 2

| | Hole transporting zone | | | | | | | | | | |
|---|---|---|---|---|---|---|---|---|---|---|---|
| | | | | | | | | Third anode side | | | Emitting region |
| | | | | | Second anode side organic layer | | | organic layer | | Difference in | First |
| | First anode side organic layer | | | | Second hole | | | Third hole | | refractive | emitting layer |
| | First | Second | Refractive | Film | transporting | Refractive | Film | transporting | Film | index | First |
| | organic | organic | index | thickness | zone | index | thickness | zone | thickness | NM₁- | host |
| | material | material | NM₁ | [nm] | material | NM₂ | [nm] | material | [nm] | NM₂ | material |
| Ex. 2 | HA | HT10 | 1.99 | 10 | HT20 | 1.80 | 40 | EBL10 | 10 | 0.19 | BH10 |
| Ex. 3 | HA | HT11 | 1.99 | 10 | HT21 | 1.79 | 40 | EBL11 | 10 | 0.20 | BH11 |
| Ex. 4 | HA | HT10 | 1.99 | 10 | HT22 | 1.84 | 40 | EBL10 | 10 | 0.15 | BH12 |
| Ex. 5 | HA | HT10 | 1.99 | 10 | HT23 | 1.83 | 40 | EBL11 | 10 | 0.16 | BH13 |
| Comp. 4 | HA | HT10 | 1.99 | 10 | HT10 | 1.99 | 40 | EBL11 | 10 | 0.00 | BH13 |

| | Emitting region | | | Electron transporting zone | | | | | | |
|---|---|---|---|---|---|---|---|---|---|---|
| | First | | | First electron | | Second electron | | | | |
| | emitting layer | | | transporting layer | | transporting layer | | | | |
| | First | | | | Film | | Film | Evaluation on Device | | |
| | additional | First luminescent | Film thickness | Compound | thickness | Compound | thickness | Voltage | EQE | LT95 |
| | host material | compound | [nm] | Name | [nm] | Name | [nm] | [V] | [%] | [hr] |
| Ex. 2 | BH20 | BD10 | 25 | HBL10 | 10 | ET10, Li | 15 | 3.6 | 9.8 | 250 |
| Ex. 3 | BH21 | BD11 | 25 | HBL10 | 10 | ET10, Li | 15 | 4.0 | 10.2 | 240 |
| Ex. 4 | BH22 | BD12 | 25 | HBL10 | 10 | ET10, Li | 15 | 3.5 | 9.2 | 250 |
| Ex. 5 | BH23 | BD13 | 25 | HBL10 | 10 | ET10, Li | 15 | 3.6 | 9.4 | 230 |
| Comp. 4 | BH23 | BD13 | 25 | HBL10 | 10 | ET10, Li | 15 | 3.5 | 8.2 | 210 |

As for Examples 2 to 5 and Comparative Example 4 each in which the hole transporting zone included the first, second, and third anode side organic layers and the first emitting layer contained two types of the host material (first host material and first additional host material), Examples 2 to 5 satisfying the relationship of the numerical formula NM (NM₁>NM₂) emitted light with higher efficiency and had a longer life than Comparative Example 4 not satisfying the relationship of the numerical formula NM.

### Evaluation on Compounds

### Singlet Energy S₁

A toluene solution of a measurement target compound at a concentration of 10 μmol/L was prepared and put in a quartz cell. An absorption spectrum (ordinate axis: absorption intensity, abscissa axis: wavelength) of the thus-obtained sample was measured at a normal temperature (300K). A tangent was drawn to the fall of the absorption spectrum close to the long-wavelength region, and a wavelength value λ_{edge} [nm] at an intersection of the tangent and the abscissa axis was assigned to a conversion equation (F2) below to calculate singlet energy.

$${S}_{1} [ eV ] = 1239 \text{.85} / {λ}_{e d g e} \qquad Conversion Equation ( F2 )$$

A spectrophotometer (U3310 produced by Hitachi, Ltd.) was used for measuring absorption spectrum.

The tangent to the fall of the absorption spectrum close to the long-wavelength region is drawn as follows. While moving on a curve of the absorption spectrum from the local maximum value closest to the long-wavelength region, among the local maximum values of the absorption spectrum, in a long-wavelength direction, a tangent at each point on the curve is checked. An inclination of the tangent is decreased and increased in a repeated manner as the curve falls (i.e., a value of the ordinate axis is decreased). A tangent drawn at a point where the inclination of the curve is the local minimum closest to the long-wavelength region (except when absorbance is 0.1 or less) is defined as the tangent to the fall of the absorption spectrum close to the long-wavelength region.

The local maximum absorbance of 0.2 or less is not counted as the above-mentioned local maximum absorbance closest to the long-wavelength region.

### Triplet Energy T₁

A measurement target compound was dissolved in EPA (diethylether:isopentane:ethanol=5:5:2 in volume ratio) at a concentration of 10 μmol/L to prepare a solution, and the solution was put in a quartz cell to provide a measurement sample. A phosphorescence spectrum (ordinate axis: phosphorescent luminous intensity, abscissa axis: wavelength) of the measurement sample was measured at a low temperature (77K). A tangent was drawn to the rise of the phosphorescence spectrum close to the short-wavelength region. An energy amount was calculated by a conversion equation (F1) below on a basis of a wavelength value λ_{edge} [nm] at an intersection of the tangent and the abscissa axis. The calculated energy amount was defined as triplet energy T₁. It should be noted that the triplet energy T₁ may have an error of about plus or minus 0.02 eV depending on measurement conditions.

$${T}_{1} [ eV ] = 1239 \text{.85} / {λ}_{e d g e} \qquad Conversion Equation ( F1 )$$

The tangent to the rise of the phosphorescence spectrum close to the short-wavelength region is drawn as follows. While moving on a curve of the phosphorescence spectrum from the short-wavelength region to the local maximum value closest to the short-wavelength region among the local maximum values of the phosphorescence spectrum, a tangent is checked at each point on the curve toward the long-wavelength of the phosphorescence spectrum. An inclination of the tangent is increased along the rise of the curve (i.e., a value of the ordinate axis is increased). A tangent drawn at a point of the local maximum inclination (i.e., a tangent at an inflection point) is defined as the tangent to the rise of the phosphorescence spectrum close to the short-wavelength region.

A local maximum point where a peak intensity is 15% or less of the maximum peak intensity of the spectrum is not counted as the above-mentioned local maximum peak intensity closest to the short-wavelength region. The tangent drawn at a point that is closest to the local maximum peak intensity closest to the short-wavelength region and where the inclination of the curve is the local maximum is defined as a tangent to the rise of the phosphorescence spectrum close to the short-wavelength region.

For phosphorescence measurement, a spectrophotofluorometer body F-4500 (produced by Hitachi High-Technologies Corporation) was used.

### Measurement of Maximum Fluorescence Peak Wavelength (FL-Peak)

A measurement target compound was dissolved in toluene at a concentration of 4.9×10⁻⁶ mol/L to prepare a toluene solution thereof. Using a fluorescence spectrometer (spectrophotofluorometer F-7000 manufactured by Hitachi High-Tech Science Corporation), the toluene solution of the measurement target compound was excited at 390 nm, where a maximum fluorescence peak wavelength A (unit: nm) was measured.

Table 3 shows measurement values of the singlet energy S₁, the triplet energy T₁ and the maximum fluorescence peak wavelength A of the compounds used for the preparation of the organic EL devices.

TABLE 3

| | S₁ [eV] | T₁ [eV] | λ [nm] |
|---|---|---|---|
| BH-2 | 3.01 | 1.82 | — |
| BH-4 | 3.01 | 1.86 | — |
| BH10 | 3.01 | 1.82 | — |
| BH11 | 3.01 | 1.86 | — |
| BH12 | 3.04 | 1.85 | — |
| BH13 | 3.01 | 1.90 | — |
| BH20 | 3.03 | 1.84 | — |
| BH21 | 3.02 | 1.82 | — |
| BH22 | 3.02 | 1.87 | — |
| BH23 | 3.00 | 1.85 | — |
| BD-4 | 2.81 | 2.30 | 450 |
| BD10 | 2.73 | 2.29 | 453 |
| BD11 | 2.71 | 2.64 | 455 |
| BD12 | 2.74 | 2.30 | 450 |
| BD13 | 2.74 | 2.65 | 451 |

### Refractive Index

The refractive index of the constituent materials (compounds) forming the organic layer was measured as follows.

A measurement target material was vacuum-deposited on a glass substrate to form a film having an approximately 50 nm thickness. Using a spectroscopic ellipsometer (M-2000U1, produced by J. A. Woollam Co., Inc. (US)), the obtained sample film was irradiated with incident light (from ultraviolet light through visible light to near-infrared light) every 5 degrees in a measurement angle range of 45 degrees to 75 degrees to measure change in a deflection state of the light reflected by the sample surface. In order to improve the measurement accuracy of the extinction coefficient, a transmission spectrum in a substrate normal direction (direction perpendicular to a surface of the substrate of the organic EL device) was also measured by M-2000U1. Similarly, the same measurement was performed also on the glass substrate on which no measurement target material was vapor-deposited. The obtained measurement information was fitted using analysis software (Complete EASE) produced by J. A. Woollam Co., Inc.

Refractive indices in an in-plane direction and a normal direction, extinction coefficients in the in-plane direction and the normal direction, and an order parameter of an organic film formed on the substrate were calculated under fitting conditions of using an anisotropic model rotationally symmetric about one axis and setting a parameter MSE indicating a mean square error in the analysis software to be 3.0 or less. A peak close to the long-wavelength region of the extinction coefficient (in-plane direction) was defined as S₁, and the order parameter was calculated by a peak wavelength of S₁. As fitting conditions for the glass substrate, an isotropic model was used.

Typically, a film formed by vacuum-depositing a low molecular material on the substrate is rotationally symmetric about one axis extending along the substrate normal direction. When an angle formed by the substrate normal direction and a molecular axis in a thin film formed on the substrate is defined as θ and the extinction coefficients in a substrate parallel direction (Ordinary direction) and a substrate perpendicular direction (Extra-Ordinary direction) obtained by performing the variable-angle spectroscopic ellipsometry measurement on the thin film are respectively defined as ko and ke, S′ represented by a formula below is the order parameter.

$$\begin{matrix}S ’ = 1 - < cos 2 θ >= 2 ko / ( ke + 2 k o ) = 2 / 3 ( 1 - S ) \\ S = ( 1 / 2 ) < 3 cos 2 θ - 1 > = ( k e - ko ) / ( ke + 2 k o )\end{matrix}$$

An evaluation method of the molecular orientation is a publicly known method, and details thereof are described in Organic Electronics, volume 10, page 127 (2009). Further, the method for forming the thin film is a vacuum deposition method.

The order parameter S′ obtained by the variable-angle spectroscopic ellipsometry measurement is 1.0 when all molecules are oriented in parallel with the substrate. When molecules are random without being oriented, the order parameter S′ is 0.66.

Herein, a value at 2.7 eV in the substrate parallel direction (Ordinary direction), from among the values measured above, is defined as a refractive index of the measurement target material.

When a layer was formed from a constituent material containing a plurality of compounds, a refractive index of the constituent material of the layer, the layer being a film formed by co-depositing the plurality of compounds as the measurement target material on the glass substrate or a film formed by vapor-depositing a mixture containing the plurality of compounds, was measured using a spectroscopic ellipsometer in the same manner as above.

Tables 1 and 2 show the refractive indices of the constituent materials (compounds) of the first and second anode side organic layers, and the differences in refractive index NM₁−NM₂ based on respective combinations of the constituent materials of the first and second anode side organic layers.

### EXPLANATION OF CODES

1, 1A, 1B, 1C, 1D, 1E, 10A, 10B . . . organic EL device; 10, 11, 12, 13, 14, 15 . . . organic layer, 2 . . . substrate, 3 . . . anode, 4 . . . cathode, 5, 5B, 6210 . . . emitting region, 51 . . . first emitting layer, 52 . . . second emitting layer, 51B . . . first emitting unit, 52B, 52C . . . second emitting unit, 61 . . . first anode side organic layer, 62 . . . second anode side organic layer, 63 . . . third anode side organic layer, 64 . . . fourth anode side organic layer, 70 . . . first charge generating layer, 8, 631 . . . electron transporting layer, 9, 641 . . . electron injecting layer, 611 . . . first cathode side organic layer, 612 . . . second cathode side organic layer, 613 . . . third cathode side organic layer, 621 . . . third emitting layer

1: An organic electroluminescence device, comprising:
a cathode;
an anode;
an emitting region disposed between the cathode and the anode; and
a hole transporting zone disposed between the anode and the emitting region, wherein
the emitting region comprises one or more emitting layers,
the one or more emitting layers comprise a first emitting layer,
the first emitting layer comprises a first host material, a first additional host material different from the first host material, and a first luminescent compound,
the hole transporting zone comprises a first anode side organic layer and a second anode side organic layer,
the first anode side organic layer is in direct contact with the second anode side organic layer,
the first anode side organic layer and the second anode side organic layer are disposed between the anode and the emitting region in this order from a side close to the anode,
the first anode side organic layer comprises a first organic material and a second organic material,
the first organic material is different from the second organic material,
a content of the first organic material in the first anode side organic layer is less than 50 mass %,
the second organic material is a monoamine compound having one substituted or unsubstituted amino group in a molecule, or a diamine compound having two substituted or unsubstituted amino groups in a molecule,
the second anode side organic layer comprises a second hole transporting zone material,
the second hole transporting zone material is a monoamine compound having one substituted or unsubstituted amino group in a molecule, or a diamine compound having two substituted or unsubstituted amino groups in a molecule,
the second hole transporting zone material is different from the first organic material,
the second hole transporting zone material and the second organic material are mutually the same or different,
the first emitting layer is an emitting layer that emits fluorescence, and
a refractive index NM₁ of constituent materials comprised in the first anode side organic layer and a refractive index NM₂ of a constituent material comprised in the second anode side organic layer satisfy a relationship of a numerical formula below (Numerical Formula NM),
$${NM}_{1} > N {M}_{2} . \qquad ( Numerical Formula NM )$$

2: The organic electroluminescence device according to claim 1, wherein the first anode side organic layer comprises no compound comprised in the second anode side organic layer.

3: The organic electroluminescence device according to claim 1, wherein a content of the first organic material in the first anode side organic layer is 15 mass % or less.

4: The organic electroluminescence device according to claim 1, wherein the first anode side organic layer is in direct contact with the anode.

5: The organic electroluminescence device according to claim 1, wherein the one or more emitting layers in the emitting region are each an emitting layer that emits fluorescence.

6: The organic electroluminescence device according to claim 1, wherein a difference NM₁−NM₂ between the refractive index NM₁ of the constituent materials comprised in the first anode side organic layer and the refractive index NM₂ of the constituent material comprised in the second anode side organic layer satisfies a relationship of a numerical formula below (Numerical Formula NM₁),
$${NM}_{1} - {NM}_{2} \geq 0.01 . \qquad ( Numerical Formula NM1 )$$

7: The organic electroluminescence device according to claim 1, wherein a difference NM₁−NM₂ between the refractive index NM₁ of the constituent materials comprised in the first anode side organic layer and the refractive index NM₂ of the constituent material comprised in the second anode side organic layer satisfies a relationship of a numerical formula below (Numerical Formula NM₂),
NM₁−NM₂≥0.05 (Numerical Formula NM₂).

8: The organic electroluminescence device according to claim 1, wherein a difference NM₁−NM₂ between the refractive index NM₁ of the constituent materials comprised in the first anode side organic layer and the refractive index NM₂ of the constituent material comprised in the second anode side organic layer satisfies a relationship of a numerical formula below (Numerical Formula NM3),
$${NM}_{1} - {NM}_{2} \geq 0.075 . \qquad ( Numerical Formula NM3 )$$

9: The organic electroluminescence device according to claim 1, wherein the refractive index NM₁ of the constituent materials comprised in the first anode side organic layer and the refractive index NM₂ of the constituent material comprised in the second anode side organic layer satisfy a relationship of a numerical formula below (Numerical Formula NM4),
$${NM}_{1} - {NM}_{2} \geq 0.1 . \qquad ( Numerical Formula NM4 )$$

10: The organic electroluminescence device according to claim 1, wherein the refractive index NM₂ of the constituent material comprised in the second anode side organic layer is 1.89 or less.

11: The organic electroluminescence device according to claim 1, wherein the refractive index NM₁ of the constituent materials comprised in the first anode side organic layer is 1.90 or more.

12: The organic electroluminescence device according to claim 1, wherein the refractive index NM₁ of the constituent materials comprised in the first anode side organic layer is 1.94 or more.

13: The organic electroluminescence device according to claim 1, wherein the emitting region consists of the first emitting layer.

14: The organic electroluminescence device according to claim 1, wherein the emitting region comprises the first emitting layer and further comprises a second emitting layer.

15: The organic electroluminescence device according to claim 14, wherein the emitting region consists of the first emitting layer and the second emitting layer.

16: The organic electroluminescence device according to claim 1, wherein the first anode side organic layer has a film thickness of 20 nm or less.

17: The organic electroluminescence device according to claim 1, wherein the second anode side organic layer has a film thickness in a range from 30 nm to 150 nm.

18: The organic electroluminescence device according to claim 1, wherein the second anode side organic layer has a film thickness in a range from 30 nm to 80 nm.

19: The organic electroluminescence device according to claim 1, wherein the second anode side organic layer has a film thickness in a range from 80 nm to 150 nm.

20: The organic electroluminescence device according to claim 1, wherein the second hole transporting zone material is a monoamine compound having one substituted or unsubstituted amino group in a molecule.

21: The organic electroluminescence device according to claim 1, wherein the second hole transporting zone material is at least one compound selected from the group consisting of a compound represented by a formula (C1) below and a compound represented by a formula (C3) below, 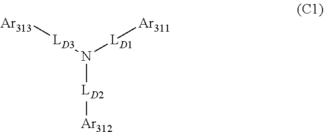
where, in the formula (C1):
Ar₃₁₁, Ar₃₁₂, and Ar₃₁₃ are each independently a substituted or unsubstituted aryl group having 6 to 50 ring carbon atoms, a substituted or unsubstituted heterocyclic group having 5 to 50 ring atoms, or —Si(R_{C1})(R_{C2})(R_{C3});
R_{C1}, R_{C2}, and R_{C3} are each independently a substituted or unsubstituted aryl group having 6 to 50 ring carbon atoms;
when a plurality of R_{C1} are present, the plurality of R_{C1} are mutually the same or different;
when a plurality of R_{C2} are present, the plurality of R_{C2} are mutually the same or different;
when a plurality of R_{C3} are present, the plurality of R_{C3} are mutually the same or different; and
L_{D1}, L_{D2}, and L_{D3} are each independently a single bond, a substituted or unsubstituted arylene group having 6 to 50 ring carbon atoms, or a substituted or unsubstituted divalent heterocyclic group having 5 to 50 ring atoms, 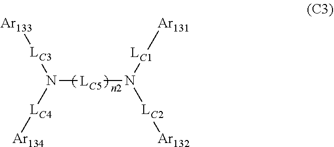
where, in the formula (C3):
L_{C1}, L_{C2}, L_{C3}, and L_{C4} are each independently a single bond, a substituted or unsubstituted arylene group having 6 to 50 ring carbon atoms, or a substituted or unsubstituted divalent heterocyclic group having 5 to 50 ring atoms;
n2 is 1, 2, 3, or 4;
when n2 is 1, L_{C5} is a substituted or unsubstituted arylene group having 6 to 50 ring carbon atoms, or a substituted or unsubstituted divalent heterocyclic group having 5 to 50 ring atoms;
when n2 is 2, 3, or 4, a plurality of L_{C5} are mutually the same or different;
when n2 is 2, 3, or 4, a plurality of L_{C5} are mutually bonded to form a substituted or unsubstituted monocyclic ring, mutually bonded to form a substituted or unsubstituted fused ring, or not mutually bonded;
L_{C5} forming neither the substituted or unsubstituted monocyclic ring nor the substituted or unsubstituted fused ring is a substituted or unsubstituted arylene group having 6 to 50 ring carbon atoms, or a substituted or unsubstituted divalent heterocyclic group having 5 to 50 ring atoms;
Ar₁₃₁, Ar₁₃₂, Ar₁₃₃, and Ar₁₃₄ are each independently a substituted or unsubstituted aryl group having 6 to 50 ring carbon atoms, a substituted or unsubstituted heterocyclic group having 5 to 50 ring atoms, or —Si(R_{C1})(R_{C2})(R_{C3});
R_{C1}, R_{C2}, and R_{C3} are each independently a substituted or unsubstituted aryl group having 6 to 50 ring carbon atoms;
when a plurality of R_{C1} are present, the plurality of R_{C1} are mutually the same or different;
when a plurality of R_{C2} are present, the plurality of R_{C2} are mutually the same or different;
when a plurality of R_{C3} are present, the plurality of R_{C3} are mutually the same or different; and
a first amino group represented by a formula (C3-1) below and a second amino group represented by a formula (C3-2) below are an identical group, 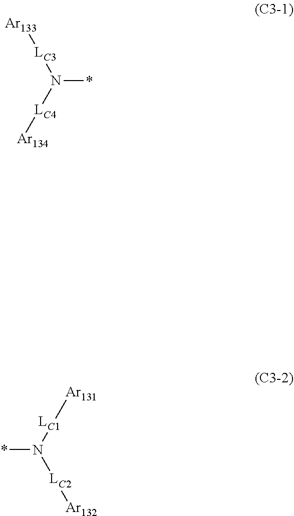
where, in the formulae (C3-1) and (C3-2),
* each represent a bonding position to L_{C5}; and
in the compound represented by the formula (C3), the substituent for the “substituted or unsubstituted” group is not a group represented by —N(R_{C6})(R_{C7}), in which R_{C6} and R_{C7} are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

22: The organic electroluminescence device according to claim 21, wherein the compound represented by the formula (C1) is at least one compound selected from the group consisting of a compound represented by a formula (cHT3-1), a compound represented by a formula (cHT3-2), a compound represented by a formula (cHT3-3), and a compound represented by a formula (cHT3-4) below, 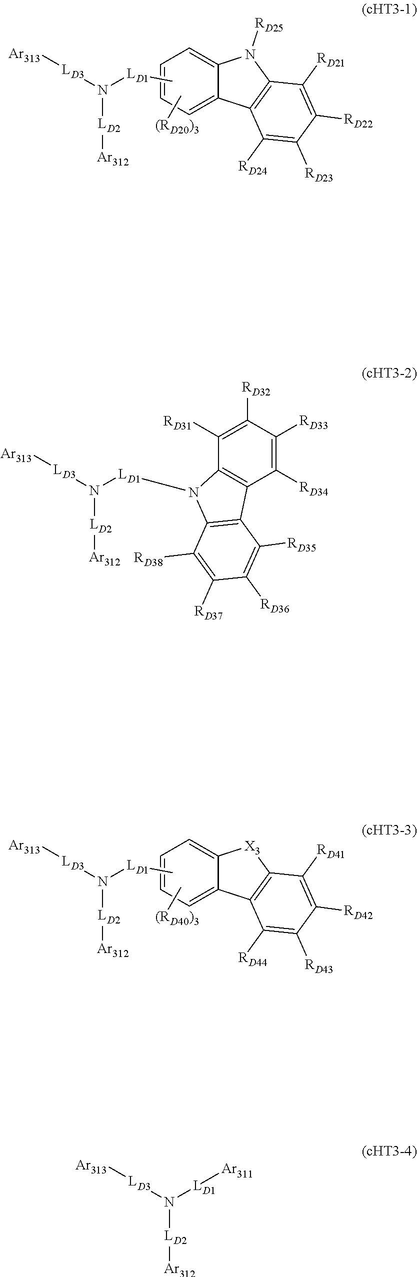
wherein, in the formulae (cHT3-1), (cHT3-2), (cHT3-3), and (cHT3-4):
Ar₃₁₂, Ar₃₁₃, L_{D1}, L_{D2}, and L_{D3} each independently represent the same as Ar₃₁₂, Ar₃₁₃, L_{D1}, L_{D2}, and L_{D3} in the formula (C1);
Ar₃₁₁ is a group represented by one of a formula (1-a), a formula (1-b), a formula (1-c), and a formula (1-d) below;
at least one combination of adjacent two or more of R_{D20} to R_{D24} are mutually bonded to form a substituted or unsubstituted monocyclic ring, mutually bonded to form a substituted or unsubstituted fused ring, or not mutually bonded;
at least one combination of adjacent two or more of R_{D31} to R_{D38} are mutually bonded to form a substituted or unsubstituted monocyclic ring, mutually bonded to form a substituted or unsubstituted fused ring, or not mutually bonded;
at least one combination of adjacent two or more of R_{D40} to R_{D44} are mutually bonded to form a substituted or unsubstituted monocyclic ring, mutually bonded to form a substituted or unsubstituted fused ring, or not mutually bonded;
X₃ is an oxygen atom, a sulfur atom, or C(R_{D45})(R_{D46});
a combination of R_{D45} and R_{D46} are mutually bonded to form a substituted or unsubstituted monocyclic ring, mutually bonded to form a substituted or unsubstituted fused ring, or not mutually bonded; and
R_{D25}, and R_{D20} to R_{D24}, R_{D31} to R_{D38}, R_{D40} to R_{D44}, R_{D45} and R_{D46} forming neither the substituted or unsubstituted monocyclic ring nor the substituted or unsubstituted fused ring are each independently a hydrogen atom, a cyano group, a substituted or unsubstituted alkyl group having 1 to 50 carbon atoms, a substituted or unsubstituted alkyl halide group having 1 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a substituted or unsubstituted aryl group having 6 to 50 ring carbon atoms, or a substituted or unsubstituted heterocyclic group having 5 to 50 ring atoms; and
in the compounds represented by the formulae (cHT3-1), (cHT3-2), (cHT3-3), and (cHT3-4),
R₉₀₁ to R₉₀₄ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different; and
when a plurality of R₉₀₄ are present, the plurality of R₉₀₄ are mutually the same or different, 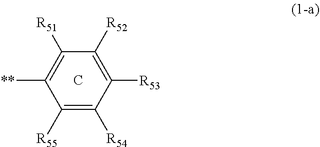
where, in the formula (1-a):
none of a combination(s) of adjacent two or more of R₅₁ to R₅₅ are bonded to each other;
R₅₁ to R₅₅ are each independently a hydrogen atom or a substituted or unsubstituted alkyl group having 1 to 6 carbon atoms; and
** represents a bonding position to L_{D1}, 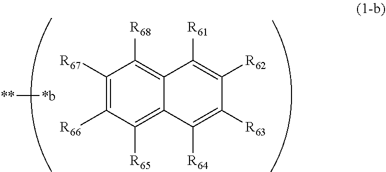
where, in the formula (1-b):
one of R₆₁ to R₆₈ is a single bond with *b;
none of a combination(s) of adjacent two or more of R₆₁ to R₆₈ not being the single bond with *b are bonded to each other;
R₆₁ to R₆₈ not being the single bond with *b are each independently a hydrogen atom, a substituted or unsubstituted alkyl group having 1 to 6 carbon atoms, or a substituted or unsubstituted aryl group having 6 to 12 ring carbon atoms; and
** represents a bonding position to L_{D1}, 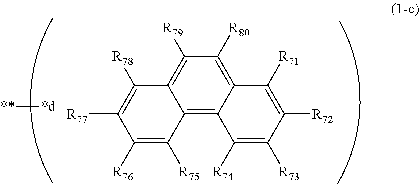
where, in the formula (1-c):
one of R₇₁ to R₈₀ is a single bond with *d;
none of a combination(s) of adjacent two or more of R₇₁ to R₈₀ not being the single bond with *d are bonded to each other;
R₇₁ to R₈₀ not being the single bond with *d are each independently a hydrogen atom, a substituted or unsubstituted alkyl group having 1 to 6 carbon atoms, or a substituted or unsubstituted aryl group having 6 to 12 ring carbon atoms; and
** represents a bonding position to L_{D1}, 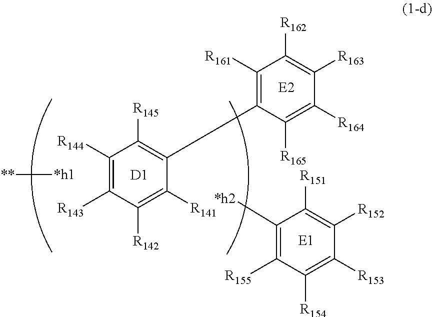
where, in the formula (1-d):
one of R₁₄₁ to R₁₄₅ is a single bond with *h1, and another one of R₁₄₁ to R₁₄₅ is a single bond with *h2;
none of a combination(s) of adjacent two or more of R₁₄₁ to R₁₄₅ not being the single bond with *h1 and not being the single bond with *h2 are bonded to each other;
at least one combination of adjacent two or more of R₁₅₁ to R₁₅₅ are mutually bonded to form a substituted or unsubstituted monocyclic ring, mutually bonded to form a substituted or unsubstituted fused ring, or not mutually bonded;
at least one combination of adjacent two or more of R₁₆₁ to R₁₆₅ are mutually bonded to form a substituted or unsubstituted monocyclic ring, mutually bonded to form a substituted or unsubstituted fused ring, or not mutually bonded;
R₁₄₁ to R₁₄₅ not being the single bond with *h1 and not being the single bond with *h2 as well as R₁₅₁ to R₁₅₅ and R₁₆₁ to R₁₆₅ forming neither the substituted or unsubstituted monocyclic ring nor the substituted or unsubstituted fused ring are each independently a hydrogen atom, a substituted or unsubstituted alkyl group having 1 to 6 carbon atoms, or a substituted or unsubstituted aryl group having 6 to 12 ring carbon atoms; and
** represents a bonding position to L_{D1}.

23: The organic electroluminescence device according to claim 1, wherein the second organic material is at least one compound selected from the group consisting of a compound represented by a formula (cHT2-1), a compound represented by a formula (cHT2-2), and a compound represented by a formula (cHT2-3) below, 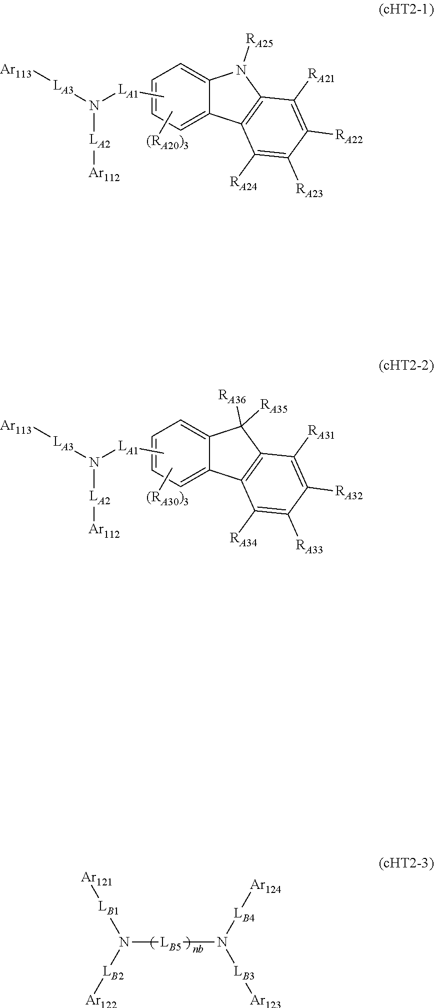
where, in the formulae (cHT2-1), (cHT2-2), and (cHT2-3):
Ar₁₁₂, Ar₁₁₃, Ar₁₂₁, Ar₁₂₂, Ar₁₂₃, and Ar₁₂₄ are each independently a substituted or unsubstituted aryl group having 6 to 50 ring carbon atoms, a substituted or unsubstituted heterocyclic group having 5 to 50 ring atoms, or —Si(R_{C1})(R_{C2})(R_{C3});
R_{C1}, R_{C2}, and R_{C3} are each independently a substituted or unsubstituted aryl group having 6 to 50 ring carbon atoms;
when a plurality of R_{C1} are present, the plurality of R_{C1} are mutually the same or different;
when a plurality of R_{C2} are present, the plurality of R_{C2} are mutually the same or different;
when a plurality of R_{C3} are present, the plurality of R_{C3} are mutually the same or different;
L_{A1}, L_{A2}, L_{A3}, L_{B1}, L_{B2}, L_{B3}, and L_{B4} are each independently a single bond, a substituted or unsubstituted arylene group having 6 to 50 ring carbon atoms, or a substituted or unsubstituted divalent heterocyclic group having 5 to 50 ring atoms;
nb is 1, 2, 3 or 4;
when nb is 1, L_{B5} is a substituted or unsubstituted arylene group having 6 to 50 ring carbon atoms, or a substituted or unsubstituted divalent heterocyclic group having 5 to 50 ring atoms;
when nb is 2, 3, or 4, a plurality of L_{B5} are mutually the same or different;
when nb is 2, 3, or 4, a plurality of L_{B5} are mutually bonded to form a substituted or unsubstituted monocyclic ring, mutually bonded to form a substituted or unsubstituted fused ring, or not mutually bonded;
L_{B5} forming neither the substituted or unsubstituted monocyclic ring nor the substituted or unsubstituted fused ring is a substituted or unsubstituted arylene group having 6 to 50 ring carbon atoms, or a substituted or unsubstituted divalent heterocyclic group having 5 to 50 ring atoms;
a combination of R_{A35} and R_{A36} are mutually bonded to form a substituted or unsubstituted monocyclic ring, mutually bonded to form a substituted or unsubstituted fused ring, or not mutually bonded;
R_{A25}, and R_{A35} and R_{A36} forming neither the substituted or unsubstituted monocyclic ring nor the substituted or unsubstituted fused ring are each independently a hydrogen atom, a cyano group, a substituted or unsubstituted alkyl group having 1 to 50 carbon atoms, a substituted or unsubstituted alkyl halide group having 1 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a substituted or unsubstituted aryl group having 6 to 50 ring carbon atoms, or a substituted or unsubstituted heterocyclic group having 5 to 50 ring atoms;
at least one combination of adjacent two or more of R_{A20} to R_{A24} are mutually bonded to form a substituted or unsubstituted monocyclic ring, mutually bonded to form a substituted or unsubstituted fused ring, or not mutually bonded;
at least one combination of adjacent two or more of R_{A30} to R_{A34} are mutually bonded to form a substituted or unsubstituted monocyclic ring, mutually bonded to form a substituted or unsubstituted fused ring, or not mutually bonded; and
R_{A20} to R_{A24} and R_{A30} to R_{A34} forming neither the substituted or unsubstituted monocyclic ring nor the substituted or unsubstituted fused ring are each independently a hydrogen atom, a cyano group, a substituted or unsubstituted alkyl group having 1 to 50 carbon atoms, a substituted or unsubstituted alkyl halide group having 1 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a substituted or unsubstituted aryl group having 6 to 50 ring carbon atoms, or a substituted or unsubstituted heterocyclic group having 5 to 50 ring atoms; and
in the compounds represented by the formulae (cHT2-1), (cHT2-2), and (cHT2-3),
R₉₀₁ to R₉₀₄ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different; and
when a plurality of R₉₀₄ are present, the plurality of R₉₀₄ are mutually the same or different.

24: The organic electroluminescence device according to claim 1, wherein the second organic material comprises at least one group selected from the group consisting of a group represented by a formula (2-a), a group represented by a formula (2-b), a group represented by a formula (2-c), a group represented by a formula (2-d), a group represented by a formula (2-e), and a group represented by a formula (2-f) below, 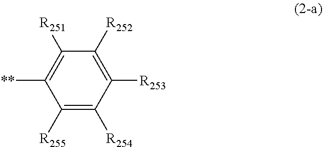
where, in the formula (2-a):
none of a combination(s) of adjacent two or more of R₂₅₁ to R₂₅₅ are bonded to each other;
R₂₅₁ to R₂₅₅ are each independently a hydrogen atom, or a substituted or unsubstituted alkyl group having 1 to 6 carbon atoms; and
** represents a bonding position, 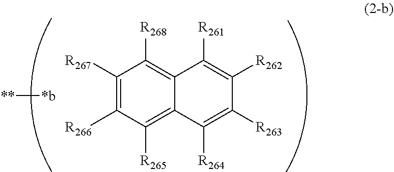
where, in the formula 2-b):
one of R₂₆₁ to R₂₆₈ is a single bond with *b;
none of a combination(s) of adjacent two or more of R₂₆₁ to R₂₆₈ not being the single bond with *b are bonded to each other;
R₂₆₁ to R₂₆₈ not being the single bond with *b are each independently a hydrogen atom, a substituted or unsubstituted alkyl group having 1 to 6 carbon atoms, or a substituted or unsubstituted aryl group having 6 to 12 ring carbon atoms; and
** represents a bonding position, 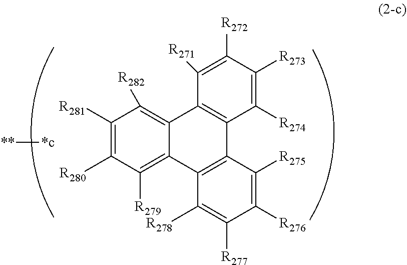
where, in the formula (2-c):
one of R₂₇₁ to R₂₈₂ is a single bond with *c;
none of a combination(s) of adjacent two or more of R₂₇₁ to R₂₈₂ not being the single bond with *c are bonded to each other;
R₂₇₁ to R₂₈₂ not being the single bond with *c are each independently a hydrogen atom, a substituted or unsubstituted alkyl group having 1 to 6 carbon atoms, or a substituted or unsubstituted aryl group having 6 to 12 ring carbon atoms; and
** represents a bonding position, 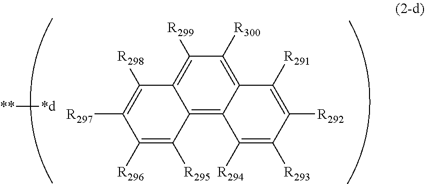
where, in the formula (2-d):
one of R₂₉₁ to R₃₀₀ is a single bond with *d;
none of a combination(s) of adjacent two or more of R₂₉₁ to R₃₀₀ not being the single bond with *d are bonded to each other;
R₂₉₁ to R₃₀₀ not being the single bond with *d are each independently a hydrogen atom, a substituted or unsubstituted alkyl group having 1 to 6 carbon atoms, or a substituted or unsubstituted aryl group having 6 to 12 ring carbon atoms; and
** represents a bonding position, 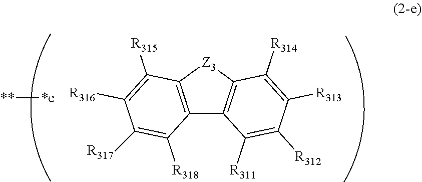
where, in the formula (2-e):
Z₃ is an oxygen atom, a sulfur atom, NR₃₁₉, or C(R₃₂₀)(R₃₂₁);
one of R₃₁₁ to R₃₂₁ is a single bond with *e, or one of carbon atoms of a substituted or unsubstituted benzene ring below formed by mutually bonding a combination of adjacent two or more of R₃₁₁ to R₃₁₈ is bonded to *e by a single bond;
a combination of adjacent two or more of R₃₁₁ to R₃₁₈ not being the single bond with *e are mutually bonded to form a substituted or unsubstituted benzene ring, or not mutually bonded;
R₃₁₁ to R₃₁₈ not being the single bond with *e and not forming the substituted or unsubstituted benzene ring are each independently a hydrogen atom, a substituted or unsubstituted alkyl group having 1 to 6 carbon atoms, a substituted or unsubstituted aryl group having 6 to 12 ring carbon atoms, or a substituted or unsubstituted heterocyclic group having 5 to 10 ring atoms;
R₃₁₉ not being the single bond with *e is a hydrogen atom, a substituted or unsubstituted alkyl group having 1 to 6 carbon atoms, or a substituted or unsubstituted aryl group having 6 to 12 ring carbon atoms;
a combination of R₃₂₀ and R₃₂₁ not being the single bond with *e are mutually bonded to form a substituted or unsubstituted monocyclic ring, mutually bonded to form a substituted or unsubstituted fused ring, or not mutually bonded;
R₃₂₀ and R₃₂₁ not being the single bond with *e, not forming the substituted or unsubstituted monocyclic ring, and not forming the substituted or unsubstituted fused ring are each independently a hydrogen atom, a substituted or unsubstituted alkyl group having 1 to 6 carbon atoms, or a substituted or unsubstituted aryl group having 6 to 12 ring carbon atoms; and
** represents a bonding position, 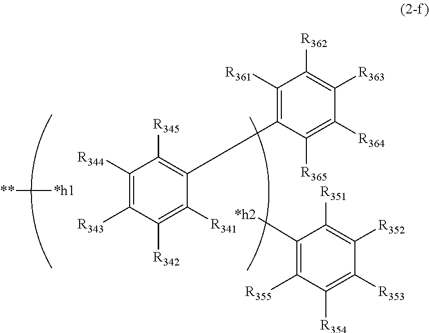
where, in the formula (2-f):
one of R₃₄₁ to R₃₄₅ is a single bond with *h1, and another one of R₃₄₁ to R₃₄₅ is a single bond with *h2;
none of a combination(s) of adjacent two or more of R₃₄₁ to R₃₄₅ not being the single bond with *h1 and not being the single bond with *h2 are bonded to each other;
at least one combination of adjacent two or more of R₃₅₁ to R₃₅₅ are mutually bonded to form a substituted or unsubstituted monocyclic ring, mutually bonded to form a substituted or unsubstituted fused ring, or not mutually bonded;
at least one combination of adjacent two or more of R₃₆₁ to R₃₆₅ are mutually bonded to form a substituted or unsubstituted monocyclic ring, mutually bonded to form a substituted or unsubstituted fused ring, or not mutually bonded;
R₃₄₁ to R₃₄₅ not being the single bond with *h1 and not being the single bond with *h2 as well as R₃₅₁ to R₃₅₅ and R₃₆₁ to R₃₆₅ forming neither the substituted or unsubstituted monocyclic ring nor the substituted or unsubstituted fused ring are each independently a hydrogen atom, a substituted or unsubstituted alkyl group having 1 to 6 carbon atoms, or a substituted or unsubstituted aryl group having 6 to 12 ring carbon atoms; and
** represents a bonding position.

25: The organic electroluminescence device according to claim 1, wherein the second organic material is a monoamine compound having one substituted or unsubstituted amino group in a molecule.

26: The organic electroluminescence device according to claim 24, wherein
the second organic material is a monoamine compound having one substituted or unsubstituted amino group in a molecule, and
the group represented by the formula (2-a), the group represented by the formula (2-b), the group represented by the formula (2-c), the group represented by the formula (2-d), the group represented by the formula (2-e), and the group represented by the formula (2-f) are each independently bonded directly, through a phenylene group, or through a biphenylene group to a nitrogen atom of an amino group of the monoamine compound.

27: The organic electroluminescence device according to claim 1, wherein the hole transporting zone further comprises a third anode side organic layer, and
the third anode side organic layer is disposed between the second anode side organic layer and the emitting region.

28: The organic electroluminescence device according to claim 27, wherein the second anode side organic layer is in direct contact with the third anode side organic layer.

29: The organic electroluminescence device according to claim 27, wherein the third anode side organic layer is in direct contact with the emitting region.

30: The organic electroluminescence device according to claim 1, wherein the first luminescent compound emits light having a maximum peak wavelength of 500 nm or less.

31: The organic electroluminescence device according to claim 30, wherein a full width at half maximum of a maximum peak of the first luminescent compound is in a range from 1 nm to 30 nm.

32: The organic electroluminescence device according to claim 1, wherein the first luminescent compound is a compound represented by a formula (6) below, 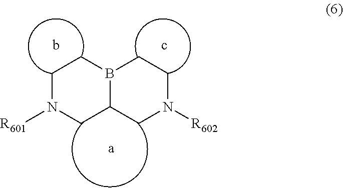
where, in the formula (6):
a ring a, a ring b and a ring c are each independently a substituted or unsubstituted aromatic hydrocarbon ring having 6 to 50 ring carbon atoms, or a substituted or unsubstituted heterocycle having 5 to 50 ring atoms;
R₆₀₁ and R₆₀₂ are each independently bonded to the ring a, the ring b or the ring c to form a substituted or unsubstituted heterocycle, or not bonded thereto to form no substituted or unsubstituted heterocycle; and
R₆₀₁ and R₆₀₂ not forming the substituted or unsubstituted heterocycle are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

33: The organic electroluminescence device according to claim 1, wherein the first host material is a compound represented by a formula (H10) below, 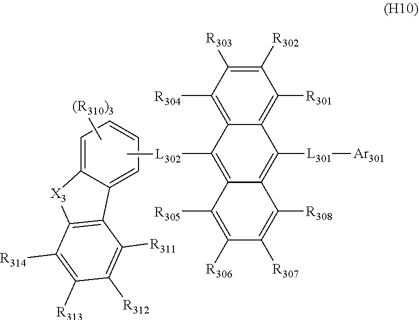
where, in the formula (H10):
X₃ is an oxygen atom or a sulfur atom;
at least one combination of adjacent two or more of three R₃₁₀ are mutually bonded to form a substituted or unsubstituted monocyclic ring, mutually bonded to form a substituted or unsubstituted fused ring, or not mutually bonded;
at least one combination of adjacent two or more of R₃₁₁ to R₃₁₄ are mutually bonded to form a substituted or unsubstituted monocyclic ring, or mutually bonded to form a substituted or unsubstituted fused ring;
R₃₀₁ to R₃₀₈, and R₃₁₀ to R₃₁₄ forming neither the substituted or unsubstituted monocyclic ring nor the substituted or unsubstituted fused ring are each a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms;
L₃₀₁ and L₃₀₂ are each independently a single bond, a substituted or unsubstituted arylene group having 6 to 50 ring carbon atoms, or a substituted or unsubstituted divalent heterocyclic group having 5 to 50 ring atoms;
A₃₀₁ is a substituted or unsubstituted aryl group having 6 to 50 ring carbon atoms, or a substituted or unsubstituted heterocyclic group having 5 to 50 ring atoms;
in the first host material, R₉₀₁, R₉₀₂, R₉₀₃, R₉₀₄, R₉₀₅, R₉₀₆ and R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different; and
when a plurality of R₉₀₇ are present, the plurality of R₉₀₇ are mutually the same or different.

34: The organic electroluminescence device according to claim 1, wherein the first additional host material comprises at least one deuterium atom.

35: The organic electroluminescence device according to claim 1, wherein the first additional host material is a compound represented by a formula (H20) below, 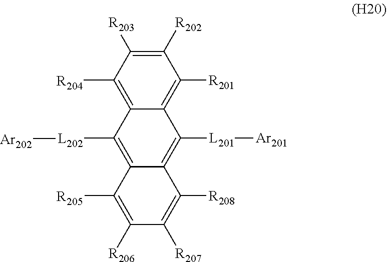
where, in the formula (H20):
R₂₀₁ to R₂₀₈ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a group represented by a formula (H21) below,
-L₂₀₃-Ar₂₀₃ (H21)
where, in the formulae (H20) and (H21):
L₂₀₁, L₂₀₂, and L₂₀₃ are each independently a single bond, or a substituted or unsubstituted arylene group having 6 to 50 ring carbon atoms; and
Ar₂₀₁, Ar₂₀₂, and Ar₂₀₃ are each independently a substituted or unsubstituted aryl group having 6 to 50 ring carbon atoms, or a substituted or unsubstituted heterocyclic group having 5 to 50 ring atoms; and
in the first additional host material,
R₉₀₁, R₉₀₂, R₉₀₃, R₉₀₄, R₉₀₅, R₉₀₆ and R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different; and
when a plurality of R₉₀₇ are present, the plurality of R₉₀₇ are mutually the same or different.

36: The organic electroluminescence device according to claim 35, wherein R₂₀₁ to R₂₀₈ each independently comprise at least one deuterium atom.

37: The organic electroluminescence device according to claim 35, wherein L₂₀₁, L₂₀₂, Ar₂₀₁, and Ar₂₀₂ each independently comprise at least one deuterium atom.

38: The organic electroluminescence device according to claim 1, wherein
the first host material substantially comprises no deuterium atom, and
the first additional host material comprises at least one deuterium atom.

39: The organic electroluminescence device according to claim 1, further comprising an electron transporting zone disposed between the cathode and the emitting region, wherein
the electron transporting zone comprises at least one electron transporting layer, and
the at least one electron transporting layer in the electron transporting zone comprises a nitrogen-containing compound having at least one of a five-membered ring containing a nitrogen atom or a six-membered ring containing a nitrogen atom.

40: The organic electroluminescence device according to claim 39, wherein the at least one electron transporting layer in the electron transporting zone comprises, as the nitrogen-containing compound, at least one compound selected from the group consisting of an imidazole derivative, a benzimidazole derivative, an azine derivative, a carbazole derivative and a phenanthroline derivative.

41: An organic electroluminescence device, comprising:
a cathode;
an anode;
an emitting region disposed between the cathode and the anode;
a hole transporting zone disposed between the anode and the emitting region; and
an electron transporting zone disposed between the cathode and the emitting region, wherein
the emitting region comprises one or more emitting layers,
the one or more emitting layers comprise a first emitting layer,
the first emitting layer comprises a first host material, a first additional host material different from the first host material, and a first luminescent compound,
the electron transporting zone comprises at least one electron transporting layer,
the hole transporting zone comprises a first anode side organic layer and a second anode side organic layer,
the first anode side organic layer is in direct contact with the second anode side organic layer,
the first anode side organic layer and the second anode side organic layer are disposed between the anode and the emitting region in this order from a side close to the anode,
the first anode side organic layer comprises a first organic material and a second organic material,
the first organic material is different from the second organic material,
a content of the first organic material in the first anode side organic layer is less than 50 mass %,
the second organic material is a monoamine compound having one substituted or unsubstituted amino group in a molecule, or a diamine compound having two substituted or unsubstituted amino groups in a molecule,
the second anode side organic layer comprises a second hole transporting zone material,
the second hole transporting zone material is a monoamine compound having one substituted or unsubstituted amino group in a molecule, or a diamine compound having two substituted or unsubstituted amino groups in a molecule,
the second hole transporting zone material is different from the first organic material,
the second hole transporting zone material and the second organic material are mutually the same or different,
the at least one electron transporting layer in the electron transporting zone comprises a phenanthroline compound having a phenanthroline skeleton,
the phenanthroline compound is a compound that has at least one group represented by a formula (21) below and is represented by a formula (20) below,
the first emitting layer is an emitting layer that emits fluorescence, and
a refractive index NM₁ of constituent materials comprised in the first anode side organic layer and a refractive index NM₂ of a constituent material comprised in the second anode side organic layer satisfy a relationship of a numerical formula below (Numerical Formula NM),
$${NM}_{1} > {NM}_{2} , \qquad ( Numerical Formula NM )$$ 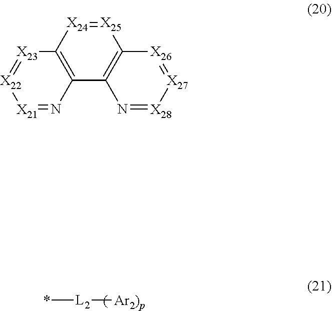
where, in the formula (20):
X₂₁ to X₂₈ are each independently a nitrogen atom, CR₂₁, or a carbon atom bonded to a group represented by the formula (21);
at least one of X₂₁ to X₂₈ is a carbon atom bonded to a group represented by the formula (21);
when a plurality of groups represented by the formula (21) are present, the plurality of groups represented by the formula (21) are mutually the same or different;
at least one combination of adjacent two or more of a plurality of R₂₁ are mutually bonded to form a substituted or unsubstituted monocyclic ring, mutually bonded to form a substituted or unsubstituted fused ring, or not mutually bonded; and
R₂₁ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₉₃₁, a group represented by —COOR₉₃₂, a group represented by —S(═O)₂R₉₃₃, a group represented by —B(R₉₃₄)(R₉₃₅), a group represented by —P(═O)(R₉₃₆)(R₉₃₇), a halogen atom, a cyano group, a nitro group, a substituted or unsubstituted aryl group having 6 to 50 ring carbon atoms, or a substituted or unsubstituted heterocyclic group having 5 to 50 ring atoms,
where, in the formula (21):
A₂ is a substituted or unsubstituted aryl group having 6 to 50 ring carbon atoms, or a substituted or unsubstituted heterocyclic group having 5 to 50 ring atoms;
p is 1, 2, 3, 4, or 5;
when two or more Ar₂ are present, the two or more Ar₂ are mutually the same or different;
L₂ is a single bond or a linking group;
L₂ as the linking group is a substituted or unsubstituted polyvalent linear, branched or cyclic aliphatic hydrocarbon group having 1 to 50 carbon atoms, a substituted or unsubstituted polyvalent aromatic hydrocarbon group having 6 to 50 ring carbon atoms, a substituted or unsubstituted polyvalent heterocyclic group having 5 to 50 ring atoms, or a polyvalent multiple linking group provided by bonding two or three groups selected from the polyvalent aromatic hydrocarbon ring group and the polyvalent heterocyclic group;
the aromatic hydrocarbon ring group and the heterocyclic group forming L₂ as the multiple linking group are mutually the same or different, and adjacent ones thereof are mutually bonded to form a ring, or not mutually bonded;
Ar₂ and L₂ as the linking group are mutually bonded to form a ring, or not mutually bonded;
L₂ as the linking group, and a carbon atom in one of X₂₁ to X₂₈ adjacent to a carbon atom bonded to L₂, or R₂₁ in CR₂₁ are mutually bonded to form a ring, or not mutually bonded; and
* in the formula (21) represents a bonding position to a ring represented by the formula (20); and
in the phenanthroline compound,
R₉₀₁, R₉₀₂, R₉₀₃, R₉₀₄, R₉₀₅, R₉₀₆, R₉₀₇, R₉₃₁, R₉₃₂, R₉₃₃, R₉₃₄, R₉₃₅, R₉₃₆ and R₉₃₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different;
when a plurality of R₉₀₇ are present, the plurality of R₉₀₇ are mutually the same or different;
when a plurality of R₉₃₁ are present, the plurality of R₉₃₁ are mutually the same or different;
when a plurality of R₉₃₂ are present, the plurality of R₉₃₂ are mutually the same or different;
when a plurality of R₉₃₃ are present, the plurality of R₉₃₃ are mutually the same or different;
when a plurality of R₉₃₄ are present, the plurality of R₉₃₄ are mutually the same or different;
when a plurality of R₉₃₅ are present, the plurality of R₉₃₅ are mutually the same or different;
when a plurality of R₉₃₆ are present, the plurality of R₉₃₆ are mutually the same or different; and
when a plurality of R₉₃₇ are present, the plurality of R₉₃₇ are mutually the same or different.

42: An organic electroluminescence device, comprising:
a cathode;
an anode;
an emitting region disposed between the cathode and the anode; and
a hole transporting zone disposed between the anode and the emitting region, wherein
the emitting region comprises one or more emitting layers,
the one or more emitting layers comprise a first emitting layer,
the first emitting layer comprises a first host material, a first additional host material different from the first host material, and a first luminescent compound,
the first host material is a compound represented by a formula (H10) below,
the first additional host material is a compound represented by a formula (H20) below,
the compound represented by the formula (H20) comprises at least one deuterium atom,
the hole transporting zone comprises a first anode side organic layer and a second anode side organic layer,
the first anode side organic layer is in direct contact with the second anode side organic layer,
the first anode side organic layer and the second anode side organic layer are disposed between the anode and the emitting region in this order from a side close to the anode,
the first anode side organic layer comprises a first organic material and a second organic material,
the first organic material is different from the second organic material,
a content of the first organic material in the first anode side organic layer is less than 50 mass %,
the second organic material is a monoamine compound having one substituted or unsubstituted amino group in a molecule, or a diamine compound having two substituted or unsubstituted amino groups in a molecule,
the second anode side organic layer comprises a second hole transporting zone material,
the second hole transporting zone material is a monoamine compound having one substituted or unsubstituted amino group in a molecule, or a diamine compound having two substituted or unsubstituted amino groups in a molecule,
the second hole transporting zone material is different from the first organic material,
the second hole transporting zone material and the second organic material are mutually the same or different,
the first emitting layer is an emitting layer that emits fluorescence, and
a refractive index NM₁ of constituent materials comprised in the first anode side organic layer and a refractive index NM₂ of a constituent material comprised in the second anode side organic layer satisfy a relationship of a numerical formula below (Numerical Formula NM),
$${NM}_{1} > {NM}_{2} , \qquad ( Numerical Formula NM )$$ 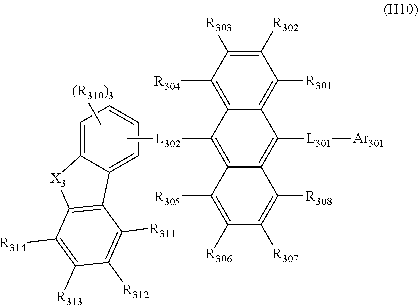
where, in the formula (H10):
X₃ is an oxygen atom or a sulfur atom;
at least one combination of adjacent two or more of three R₃₁₀ are mutually bonded to form a substituted or unsubstituted monocyclic ring, mutually bonded to form a substituted or unsubstituted fused ring, or not mutually bonded;
at least one combination of adjacent two or more of R₃₁₁ to R₃₁₄ are mutually bonded to form a substituted or unsubstituted monocyclic ring, or mutually bonded to form a substituted or unsubstituted fused ring;
R₃₀₁ to R₃₀₈, and R₃₁₀ to R₃₁₄ forming neither the substituted or unsubstituted monocyclic ring nor the substituted or unsubstituted fused ring are each a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms;
L₃₀₁ and L₃₀₂ are each independently a single bond, a substituted or unsubstituted arylene group having 6 to 50 ring carbon atoms, or a substituted or unsubstituted divalent heterocyclic group having 5 to 50 ring atoms; and
A₃₀₁ is a substituted or unsubstituted aryl group having 6 to 50 ring carbon atoms, or a substituted or unsubstituted heterocyclic group having 5 to 50 ring atoms, 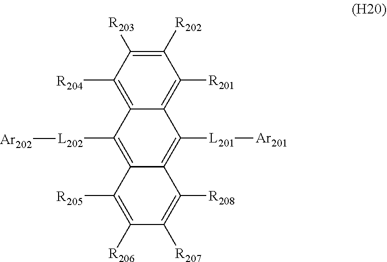
where, in the formula (H20):
R₂₀₁ to R₂₀₈ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a group represented by a formula (H21) below,
-L₂₀₃-Ar₂₀₃ (H21)
where, in the formulae (H20) and (H21):
L₂₀₁, L₂₀₂, and L₂₀₃ are each independently a single bond, or a substituted or unsubstituted arylene group having 6 to 50 ring carbon atoms; and
Ar₂₀₁, Ar₂₀₂, and Ar₂₀₃ are each independently a substituted or unsubstituted aryl group having 6 to 50 ring carbon atoms, or a substituted or unsubstituted heterocyclic group having 5 to 50 ring atoms; and
in the first host material and the first additional host material,
R₉₀₁, R₉₀₂, R₉₀₃, R₉₀₄, R₉₀₅, R₉₀₆ and R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different; and
when a plurality of R₉₀₇ are present, the plurality of R₉₀₇ are mutually the same or different.

43: The organic electroluminescence device according to claim 1, comprising:
a first emitting unit comprising the hole transporting zone as a first hole transporting zone and the emitting region as a first emitting region;
a first charge generating layer disposed between the first emitting unit and the cathode; and
a second emitting unit that is disposed between the first charge generating layer and the cathode and comprises a second hole transporting zone and a second emitting region, wherein
the first hole transporting zone, the first emitting region, the first charge generating layer, the second hole transporting zone, and the second emitting region are disposed in this order,
the second hole transporting zone comprises a first cathode side organic layer and a second cathode side organic layer,
the first cathode side organic layer is in direct contact with the second cathode side organic layer,
the first cathode side organic layer and the second cathode side organic layer are disposed between the first charge generating layer and the second emitting region in this order from a side close to the first charge generating layer,
a refractive index NL₁ of a constituent material comprised in the first cathode side organic layer and a refractive index NL₂ of a constituent material comprised in the second cathode side organic layer satisfy a relationship of a numerical formula below (Numerical Formula L),
the second emitting region comprises one or more emitting layers,
the one or more emitting layers comprise a third emitting layer,
the third emitting layer comprises a third luminescent compound, and
a maximum peak wavelength of the first luminescent compound and a maximum peak wavelength of the third luminescent compound are mutually the same or different,
$$N {L}_{1} > N {L}_{2} . \qquad ( Numerical Formula L )$$

44: The organic electroluminescence device according to claim 43, wherein the maximum peak wavelength of the first luminescent compound and the maximum peak wavelength of the third luminescent compound are mutually different.

45: The organic electroluminescence device according to claim 43, wherein the maximum peak wavelength of the first luminescent compound is shorter than the maximum peak wavelength of the third luminescent compound.

46: The organic electroluminescence device according to claim 43, wherein one or more maximum peak wavelengths of one or more of the luminescent compounds comprised in one or more of the emitting layers in the first emitting region are each shorter than one or more maximum peak wavelengths of one or more of the luminescent compounds comprised in one or more of the emitting layers in the second emitting region.

47: The organic electroluminescence device according to claim 43, wherein
the second hole transporting zone further comprises a third cathode side organic layer,
the first cathode side organic layer is in direct contact with the second cathode side organic layer, and the second cathode side organic layer is in direct contact with the third cathode side organic layer, and
the first cathode side organic layer, the second cathode side organic layer, and the third cathode side organic layer are disposed between the first charge generating layer and the second emitting region in this order from the side close to the first charge generating layer.

48: An electronic device comprising the organic electroluminescence device according to claim 1.

49: The organic electroluminescence device according to claim 41, comprising:
a first emitting unit comprising the hole transporting zone as a first hole transporting zone and the emitting region as a first emitting region;
a first charge generating layer disposed between the first emitting unit and the cathode; and
a second emitting unit that is disposed between the first charge generating layer and the cathode and comprises a second hole transporting zone and a second emitting region, wherein
the first hole transporting zone, the first emitting region, the first charge generating layer, the second hole transporting zone, and the second emitting region are disposed in this order,
the second hole transporting zone comprises a first cathode side organic layer and a second cathode side organic layer,
the first cathode side organic layer is in direct contact with the second cathode side organic layer,
the first cathode side organic layer and the second cathode side organic layer are disposed between the first charge generating layer and the second emitting region in this order from a side close to the first charge generating layer,
a refractive index NL₁ of a constituent material comprised in the first cathode side organic layer and a refractive index NL₂ of a constituent material comprised in the second cathode side organic layer satisfy a relationship of a numerical formula below (Numerical Formula L),
the second emitting region comprises one or more emitting layers,
the one or more emitting layers comprise a third emitting layer,
the third emitting layer comprises a third luminescent compound, and
a maximum peak wavelength of the first luminescent compound and a maximum peak wavelength of the third luminescent compound are mutually the same or different,
$$N {L}_{1} > N {L}_{2} . \qquad ( Numerical Formula L )$$

50: The organic electroluminescence device according to claim 42, comprising:
a first emitting unit comprising the hole transporting zone as a first hole transporting zone and the emitting region as a first emitting region;
a first charge generating layer disposed between the first emitting unit and the cathode; and
a second emitting unit that is disposed between the first charge generating layer and the cathode and comprises a second hole transporting zone and a second emitting region, wherein
the first hole transporting zone, the first emitting region, the first charge generating layer, the second hole transporting zone, and the second emitting region are disposed in this order,
the second hole transporting zone comprises a first cathode side organic layer and a second cathode side organic layer,
the first cathode side organic layer is in direct contact with the second cathode side organic layer,
the first cathode side organic layer and the second cathode side organic layer are disposed between the first charge generating layer and the second emitting region in this order from a side close to the first charge generating layer,
a refractive index NL₁ of a constituent material comprised in the first cathode side organic layer and a refractive index NL₂ of a constituent material comprised in the second cathode side organic layer satisfy a relationship of a numerical formula below (Numerical Formula L),
the second emitting region comprises one or more emitting layers,
the one or more emitting layers comprise a third emitting layer,
the third emitting layer comprises a third luminescent compound, and
a maximum peak wavelength of the first luminescent compound and a maximum peak wavelength of the third luminescent compound are mutually the same or different,
$$N {L}_{1} > N {L}_{2} . \qquad ( Numerical Formula L )$$
